(19) United States
(12) Patent Application Publication
(10) Pub. No.: US 2024/0108689 A1
(43) Pub. Date: Apr. 4, 2024

(54) MODULATION OF A PATHOGENIC PHENOTYPE IN TH1 CELLS

(71) Applicants: The Brigham and Women's Hospital, Inc., Boston, MA (US); The Broad Institute, Inc., Cambridge, MA (US); The General Hospital Corporation, Boston, MA (US); The Regents of the University of California, Oakland, CA (US)

(72) Inventors: Vijay K. Kuchroo, Boston, MA (US); Ramnik Xavier, Cambridge, MA (US); Mathias Pawlak, Boston, MA (US); David DeTomaso, Oakland, CA (US); Nir Yosef, Oakland, CA (US)

(21) Appl. No.: 18/273,579

(22) PCT Filed: Jan. 21, 2022
(86) PCT No.: PCT/US22/13331

§ 371 Date: Jul. 21, 2023

Related U.S. Application Data
(60) Provisional application No. 63/140,458, filed on Jan. 22, 2021.

(51) Int. Cl. A61K 38/17; A61P 37/02; C12N 5/0783; C12N 9/22

(52) U.S. Cl. CPC A61K 38/177; A61P 37/02; C12N 5/0636; C12N 9/22; C12N 2310/20; C12N 2501/2312; C12N 2501/2321; C12N 2501/2323; C12N 2503/02; C12N 2510/00

(57) ABSTRACT

The subject matter disclosed herein is generally directed to pathogenic Th1 cells whose phenotype is dependent on IL-23R signaling. Th1 cell specific therapeutic targets and gene programs are disclosed herein. In particular, inhibition of CD160 reduces Th1 cell pathogenicity.

### CROSS-REFERENCE TO RELATED APPLICATIONS

This application claims the benefit of U.S. Provisional Application No. 63/140,458 filed Jan. 22, 2021. The entire contents of the above-identified applications are hereby fully incorporated herein by reference.

### STATEMENT REGARDING FEDERALLY SPONSORED RESEARCH

This invention was made with government support under Grant Nos. AI073748, AI144166, AI056299, AI039671 awarded by the National Institutes of Health. The government has certain rights in the invention.

### REFERENCE TO AN ELECTRONIC SEQUENCE LISTING

The contents of the electronic sequence listing (“BROD-5340WP_ST25.txt”; Size is 59,263 bytes and it was created on Jan. 20, 2022) is herein incorporated by reference in its entirety.

### TECHNICAL FIELD

The subject matter disclosed herein is generally directed to colitogenic Th1 cells whose phenotype is dependent on IL-23R signaling.

### BACKGROUND

The cytokine IL-23 and its receptor IL-23R play a fundamental role in inducing tissue inflammation and autoimmunity⁴. It has been shown that pre-clinical models of Multiple Sclerosis, arthritis and inflammatory bowel disease (IBD) are all protected from disease in animals deficient for IL-23 signaling.¹⁻³. The relevance to human disease is emphasized by genome-wide association studies (GWAS) that established IL-23R as a risk gene in multiple human autoimmune diseases including IBD⁵ and a number of anti-IL-23 inhibitors have been approved for the treatment of Psoriasis and are now being tested in other autoimmune conditions where Th17 cells have not been implicated in disease induction¹².

IL-23R signaling is crucial for evoking a pathogenic phenotype in Th17 cells by stabilizing their function and inducing multiple factors that make Th17 cells highly pro-inflammatory¹³. Although the Th17 cell subset is most prominently associated with IL-23R function, several observations, however, have been difficult to reconcile under the assumption that IL-23R solely operates in pathogenic Th17 cells and not other pathogenic subsets. Importantly, in the pre-clinical colitis models, IL-23R signaling has also been implicated in regulating FoxP3⁺Treg function^{14, 15}. However, it has been observed that bona fide Th1 cells also elicit colitis in pre-clinical models, particularly in the adoptive transfer colitis model⁷, but Th1 cells are not known to express IL-23R. These data are conflicting with the observation that IL-23R signaling is required for the induction of colitis. Interestingly, a recent report suggests that Th17 cells need to transdifferentiate into Th1 cells to elicit colitis in this model⁷, however, whether Th1 cells themselves, driven by IL-23R, could trigger colitis without going through a Th17 cell-state has not been addressed. Clinically, secukinumab, a monoclonal antibody targeting IL-17A has been found effective in plaque psoriasis, psoriatic arthritis and ankylosing spondylitis yet ineffective in IBD⁶. In contrast, ustekinumab, a monoclonal antibody targeting both IL-12 and IL-23, and therefore targeting differentiation of both Th1 cells and Th17 cells, has proven to be effective in treating IBD^{16,17}. Indeed, the seminal studies by Powrie and colleagues have established that both Th1 and Th17 cells develop in the pre-clinical disease model of IBD through the adoptive transfer of naïve CD45RB^{hi} T cells^{3,18}. These observations raised important questions and provided impetus to investigate whether IL-23 may also confer pathogenicity to another T helper cell subset, in addition to quintessential Th17 cells, to thereby contribute to the induction of IBD.

Citation or identification of any document in this application is not an admission that such a document is available as prior art to the present invention.

### SUMMARY

In one aspect, the present invention provides for a method of treating an autoimmune disease caused by pathogenic Th1 cells comprising administering one or more agents capable of inhibiting the expression, activity and/or function of one or more genes selected from the group consisting of CD160, GPR18, GZMB, ITGB1, CCR3, GZMA, IL22, ZFP36L2, ZBTB38, CD74, CCR5, MAP3K8, TNFSF8, IFITM1, NFKBIZ, FOSL2, CREM, CCDC85B, FOS, GPR183, S100A4, 1110008F13RIK, LSP1, LITAF, CD7, DUSP2, PLAC8, H1F0, S1PR1, NCF4, SMIM3, TESC, RBMS1, LPXN, TNFRSF9, PMM1, TOB2, IFNG, CD226, and CTSW. In certain embodiments, the one or more agents inhibit the expression, activity and/or function of CD160. In certain embodiments, the one or more agents inhibit the expression, activity and/or function of GPR18. In certain embodiments, the one or more agents comprises an antibody, antibody fragment, intrabody, small molecule, small molecule degrader, antibody-like protein scaffold, aptamer, polypeptide, genetic modifying agent, or any combination thereof. In certain embodiments, the genetic modifying agent comprises an RNA-guided nuclease system, RNAi system, a zinc finger nuclease, a TALE, or a meganuclease. In certain embodiments, the RNA-guided nuclease system is a CRISPR system or IscB system. In certain embodiments, the CRISPR system comprises a CRISPR-Cas base editing system, a prime editor system, or a CAST system.

In another aspect, the present invention provides for a method of treating an autoimmune disease caused by pathogenic Th1 cells comprising administering Th1 cells modified to have decreased expression of IL-23R.

In another aspect, the present invention provides for a method of detecting a Th1 inflammatory response for diagnosis or monitoring of a treatment of a subject suffering from an autoimmune disease caused by colitogenic Th1 cells comprising detecting in a sample obtained from the subject Th1 cells expressing one or more genes selected from the group consisting of: IL23R, CD160, GPR18, GZMB, ITGB1, CCR3, GZMA, IL22, ZFP36L2, ZBTB38, CD74, CCR5, MAP3K8, TNFSF8, IFITM1, NFKBIZ, FOSL2, CREM, CCDC85B, FOS, GPR183, S100A4, 1110008F13RIK, LSP1, LITAF, CD7, DUSP2, PLAC8, H1F0, S1PR1, NCF4, SMIM3, TESC, RBMS1, LPXN, TNFRSF9, PMM1, TOB2, IFNG, CD226, and CTSW, wherein the expression of one or more genes are increased in an inflammatory response; or clusters 2, 9 and 7 in Table 3, wherein the expression of one or more genes or program from cluster 2 and/or 9 are increased in an inflammatory response and the expression of one or more genes or program in cluster 7 are decreased in an inflammatory response. In certain embodiments, the treatment comprises one or more agents capable of inhibiting the expression, activity and/or function of CD160, GPR18, IL-23, or IL-12 and IL-23. In certain embodiments, the Th1 cells are detected by immunohistochemistry (IHC), fluorescence activated cell sorting (FACS), fluorescently bar-coded oligonucleotide probes, RNA FISH (fluorescent in situ hybridization), RNA-seq, or any combination thereof. In certain embodiments, the Th1 cell expression is inferred from bulk RNA-seq. In certain embodiments, the Th1 cell expression is determined by single cell RNA-seq. In certain embodiments, the sample is obtained by biopsy.

In certain embodiments, the autoimmune disease is inflammatory bowel disease (IBD) or type 1 diabetes.

In another aspect, the present invention provides for a method of obtaining IL-23R+Th1 cells comprising differentiating naïve CD4+ T cells in vitro with IL-12 and IL-21. In certain embodiments, the method further comprises differentiating with IL-23.

In another aspect, the present invention provides for a method of treating cancer comprising administering to a subject in need thereof Th1 cells differentiated according to any embodiment herein. In certain embodiments, the naïve CD4+ T cells are obtained from the subject.

In another aspect, the present invention provides for a method of screening for drugs capable of shifting pathogenic Th1 cells to non-pathogenic Th1 cells comprising: treating Th1 cells obtained according to any embodiment herein with a drug candidate; detecting expression of one or more genes or program selected from cluster 2, 9 or 7 in Table 3; and identifying the drug, wherein the expression of one or more genes or program from cluster 2 or 9 decrease, and/or the expression of one or more genes or program in cluster 7 increase as compared to cells not contacted with the drug candidate.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-1G—IL-12+IL-21 strongly induce IL-23R in Th1 cells in vitro. FIG. 1a, Naïve CD44^{low}CD62L^{hi} CD4⁺ T cells were cultured under four different conditions in vitro for 5 days on anti-CD3/CD28 antibody-coated plates with the following conditions: TGF-β+IL-6 (non-pathogenic Th17 cells), IL-1β+IL-6+IL-23 (pathogenic Th17 cells), IL-12+IL-21 and IL-12+IL-21+IL-23. Flow cytometry was used to measure the extent of IL-23R expression taking advantage of an eGFP reporter allele. FIG. 1b, qPCR analysis shows the expression of Il23r in wildtype (Il23r^{eGFP/wt}) cells. Important signature Th1 and Th17 genes such as Ifng, Il17a, Tbx21, Rorc and Stat3 were measured in both wildtype (Il23r^{eGFP/wt}) and KO cells (Il23r^{eGFP/eGFP}) FIG. 1c, Schematic of sorting by FACS of IL-23R⁺ (eGFP⁺) and IL-23R⁻ (eGFP⁻) cells differentiated with IL-12+IL-21+IL-23 in preparation of scRNAseq (Smart-seq2). FIG. 1d, t-SNE plots showing the expression of Th1 and Th17 signature genes. FIG. 1e, Selected genes are shown that are expressed in eGFP⁺ and eGFP⁻ cells in wildtype (Il23r^{eGFP/wt}) cells. Green violin plots represent eGFP⁺-sorted cells. FIG. 1f, 528 genes were found to be differentially expressed between eGFP⁺ and eGFP⁻ cells from wildtype cells (Il23r^{eGFP/wt}) In knockout cells for IL-23R (Il23r^{eGFP/eGFP}) only 129 genes were found to be differentially expressed between eGFP⁺ and eGFP⁻ cells. FIG. 1g, Volcano plot showing genes differentially expressed in an IL-23R dependent manner. An interaction coefficient was computed to identify differences between the eGFP⁺ and eGFP⁻ populations which varied according to genotype (wildtype Il23r^{eGFP/wt} vs. knockout Il23r^{eGFP/eGFP}) Panel a is representative of several experiments and mean+s.e.m are shown. In Panel b mean+s.e.m are shown.

FIGS. 2A-2F—scRNAseq of tissue-infiltrating Th1 cells in a pre-clinical model of adoptive transfer colitis. FIG. 2a, Schematic of adoptive transfer colitis by in vitro differentiated Th1 cells followed by histopathology and 10× scRNAseq. Viable CD45⁺CD4⁺ T lymphocytes from RAG1^{-/-} recipients of either wildtype cells (Il23r^{wt/eGFP}) or knockout cells (Il23r^{eGFP/eGFP}) respectively, were isolated from the intestinal mucosa through FACS in preparation of scRNAseq. FIG. 2b, Deficiency of IL-23R protects from Th1 cell mediated adoptive transfer colitis as evaluated by histopathology. H&E staining and clinical score are shown. Mean+s.e.m. FIG. 2c, UMAP of 30,260 single cells sequenced from spleen, colonic and small intestinal (SI) lamina propria (LPL) and colonic epithelium (IEL=intraepithelial lymphocytes). FIG. 2d, Heatmap of differentially expressed genes among the tissues analyzed with selected genes highlighted as dot plots. FIG. 2e, Identification of wildtype (teal) and knockout cells (magenta), respectively. FIG. 2f, Selected genes are shown that are expressed in an IL-23R dependent manner. Red representing higher expression in wildtype cells and blue representing higher expression in knockout cells.

FIGS. 3A-3E—IL-23R drives the expansion of highly inflammatory and colitogenic T cells in the lamina propria as identified by scRNAseq. FIG. 3a, UMAP of T cells isolated from the lamina propria of small intestine and colon. Wildtype cells (teal) and knockout cells (magenta) are highlighted. FIG. 3b, Cluster analysis identifies 12 clusters with particular transcriptional signatures. FIG. 3c, The size of each cluster is shown as a percentage of the total number of cells. For each cluster, its relative abundance among wild type vs. knockout cells is visualized. FIG. 3d, Two clusters with a highly inflammatory and colitogenic signature are shown (clusters 2 and 9) which are dominated by wildtype cells. The top 20 differentially expressed genes in comparison to all other clusters are shown. Cluster 7 consists in the majority of knockout cells and exhibits a signature reminiscent of Tr-like cells FIG. 3e, Applicants assembled a list of genes (597 genes) found within IBD GWAS risk loci for which De Lange et al. (2017) provided the main basis. Applicants then asked which of these genes are expressed in clusters 2, 7 and 9 in an IL-23R-dependent manner comparing expression levels between wildtype and knockout cells. Positive values indicate higher expression in wildtype cells (Il23r^{eGFP/wt}) Asterisks indicate statistically significant differences (FDR<0.1).

FIGS. 4A-4E—Ranking algorithm and validation identify novel drivers of T cell-mediated intestinal inflammation in an IL-23R dependent manner. FIG. 4a, Identification and ranking of potential novel drivers of intestinal inflammation taking several critical considerations into account. 4 tracks are shown. Track 1: Genes are evaluated based on their expression in vivo in a cluster and genotype specific manner emphasizing clusters 2 and 9. Track 2: Genes are ranked based on their tissue specific expression comparing intestinal and peripheral (splenic) expression. Track 3: Genes are ranked based on their IL-23R dependent expression in vitro following differentiation with IL-12+IL-21+IL-23 (Smart-seq2 data from FIG. 1). Track 4: Genes that are found within an IBD GWAS risk locus are identified contributing to an increased ranking score. See methods for details of ranking. FIG. 4b, Deficiency for CD160 in Th1 cells adoptively transferred into RAG1^{-/-} recipients protects from colitis. Representative H&E histopathological stainings are shown. FIG. 4c, Intestinal inflammation and colitis are scored in the following way: 0 (healthy)-4 (most severe colitis). Pooled data from three independent experiments are shown for small intestine and proximal colon. Mean+s.e.m. are indicated. FIG. 4d, Colon length in RAG1^{-/-} recipients of either wildtype or CD160^{-/-} cells. Pooled data are shown from two independent experiments. FIG. 4e, Gzmb expression is measured by flow cytometry in both Cd160^{+/+} and Cd160^{-/-} Th1 cells prior to adoptive transfer. One experiment. Technical replicates. A t-test was used to determine statistical significance in panels c, d and e.

FIG. 5—Cytokine screen identifies IL-21 as a cytokine that together with IL-12 induces strong expression of IL-23R in Th1 cells in vitro. Applicants isolated naïve T cells from Il23r^{wt/eGFP} reporter mice and tested 19 different conditions. IL-23R expression was measured by flow cytometry identifying eGFP⁺ cells. Several conditions strongly induced IL-23R expression including the pathogenic Th17 cell condition IL-1β+IL-6+IL-23. The strongest expression of IL-17 was observed with the condition TGF-β+IL-6. Even though IL-21 and IL-6 alone induced IL-23R expression they resulted in the complete lack of IFN-γ expression and showed slight induction of IL-17A. Importantly, both IL-12+IL-21 and IL-12+IL-21+IL-23 induced strong expression of the Th1 cell signature cytokine IFN-γ and complete lack of IL-17A expression.

FIG. 6—Dot plot showing expression of 12 key genes in Th1 and Th17 cell biology across the 4 populations profiled by Smart-seq2. Tbx21, Il12rb2 and Il21r expression appeared largely unaltered by deficiency for Il23r which was consistent with unaltered expression between eGFP⁺ and eGFP⁻ populations. Of note, the expression level of Il23r in knockout cells (Il23r^{eGFP/eGFP}) is merely reminiscent of the fact that these cells produce mRNA truncated of the essential C-terminal region of IL-23R which was replaced by an IRES-eGFP sequence which results in a functional KO but is detected by Smart-seq2.

FIGS. 7A-7D—IL-23R⁺ Th1 and IL-23R⁺ Th17 cells show both common and unique transcriptional signatures. Pathogenic Th17 cells were differentiated with IL-1β+IL-6+IL-23 and IL-23R⁺ (i.e., eGFP⁺) and IL-23R⁻ (i.e., eGFP⁻) were sorted and then single-cell Smart-seq2 was performed and the transcriptional signatures were compared to Th1 cells differentiated with IL-12+IL-21+IL-23 reported in FIG. 1. FIG. 7a, Th1 cells and Th17 cells show a set of genes that is similarly regulated in both IL-23R⁺ cells from either population. FIG. 7b, Some genes are differentially regulated between IL-23R⁺ and IL-23R⁻ cells in Th1 cells only. FIG. 7c, Some genes show the exact opposite behavior in Th1 and Th17 cells. FIG. 7d, A fourth set of genes was differentially regulated between IL-23R⁺ and IL-23R⁻ cells in Th17 cells only.

FIGS. 8A-8G—Tissue infiltrating CD45⁺CD4⁺ T cells show strong expression of IFN-γ. Flow cytometric analyses of tissue infiltrating T cells isolated from the intestine of recipients of either wildtype or IL-23R KO cells and cultured ON with the cytokines IL-7+IL-23. ICC was performed after restimulation with PMA/ionomycin. FIG. 8a, Colonic LPL cells. Two recipients of wildtype cells (Il23r^{eGFP/wt}) and one recipient of KO cells (Il23r^{eGFP/eGFP}) are shown. Gated on viable CD45⁺ cells. FIG. 8b, FIG. 8c ICC for IFN-γ, IL-17A and GMCSF of the two recipients of wildtype cells is shown. FIG. 8d, Colonic IEL cells. One recipient of wildtype cells and one recipient of KO cells are shown. Gated on viable CD45⁺ cells. FIG. 8e, ICC for IFN-γ and IL-17A of the IEL samples. FIG. 8f, FIG. 8g Pooled values of LPL and IEL samples. LPL and IEL samples were isolated and analyzed in independent experiments. Data in panels f and g are mean±SD. Unpaired t-test, p value **<0.01 in panel f.

FIG. 9—Cluster 8 represents highly proliferating cells. UMAPs show that cells of cluster 8 highly express genes critical to cell cycle progression such as Cdc20, Ccnb1 (cyclin B), Cdc6, Cdk1 and the proliferative marker Mki67.

FIGS. 10A-10D—IL-23R negatively impacts the development of regulatory Tr1 cells in the intestinal lamina propria as identified by scRNAseq. FIG. 10a, UMAPs and correlated expression profiles of signature genes of regulatory Tr cells exposing cluster 7. FIG. 10b, Dot plot showing the expression of signature genes in comparison to all other clusters. FIG. 10c, Transcriptional signature of Tr regulatory cells identified by Gruarin et al. (2019) highlights cluster 7. FIG. 10d, Volcano plot highlighting differentially expressed genes between wildtype (Il23r^{eGFP/wt}) and knockout (II23r^{eGFP/eGFP}) cells within cluster 7.

FIGS. 11A-11B—In vitro differentiated Th1 cells from either Cd160^{+/+} or Cd160^{-/-} cells do not show a difference in IFN-γ expression prior to adoptive transfer. FIG. 11a, In vitro differentiated Th1 cells do not show a difference in IFN-γ production under culture with IL-12+IL-21+IL-23 between wildtype and CD160 knockout cells. One representative experiment of three independent experiments is shown. FIG. 11b, Pooled data from in vitro differentiated Th1 cells are shown prior to adoptive transfer into RAG1^{-/-} mice. 3 independent experiments. Mean is shown with SD error bars. All differences are non-significant.

FIG. 12—Overview of T cell and NK cell markers in cells isolated from the intestinal mucosa (LPL) and sequenced by 10× technology. UMAPs of cells sequenced that show uniform expression of CD45, CD3, CD4 and lack expression of NK cell markers such as NK1.1, CD56 and CD16 in addition to the lack of CD8a.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a” “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide methods of treating Th1 cell-mediated autoimmune diseases, generating IL-23R⁺ Th1 cells, and detection and modulation of pathogenic and regulatory cell programs. The cytokine receptor IL-23R plays a fundamental role in inflammation and autoimmunity¹⁻⁵. However, several observations have been difficult to reconcile under the assumption that only Th17 cells critically depend on IL-23 to acquire a pathogenic phenotype^{6,7}. To this end, Applicants performed a cytokine screen and discovered that the cytokine combination IL-12+IL-21 strongly induces IL-23R on Th1 cells in vitro. Th1 cells differentiated in vitro with IL-12+IL-21 show similar levels of IL-23R expression as in pathogenic Th17 cells^{8,9}. Applicants demonstrate that IL-23R is required for Th1 cells to acquire a highly pathogenic/colitogenic phenotype in an adoptive transfer colitis model^{3,7}. Thus, the Th1 cells were pathogenic. Loss of IL-23R expression on Th1 cells almost completely abrogated the ability of Th1 cells to transfer colitis. Thus, the Th1 cells were non-pathogenic. Using massively parallel single-cell RNA-sequencing (scRNAseq) of intestinal tissue-infiltrating colitogenic Th1 cells, Applicants demonstrated a striking expression of genes that promote inflammation and linked these genes with multiple pathways implicated in the induction of IBD in humans as identified by GWAS analysis. scRNAseq analysis of intestinal T cells enabled identification of novel regulators induced by IL-23R-signaling in Th1 cells which differed from those expressed in Th17 cells⁹⁻¹¹. Applicants identified a number of novel regulators induced by IL-23R-dependent signaling in Th1 cells and demonstrated a critical role for the IL-23R-dependent gene Cd160 in colitis. The perturbation of CD160 in Th1 cells inhibited induction of colitis, validating the role of IL-23R signaling in Th1 cells.

Applicants were able to uncouple IL-23R as a purely Th17 cell-specific factor and implicate IL-23R signaling as a pathogenic driver of Th1 cell-mediated tissue inflammation and disease. Thus, Applicants identified a pathogenic function for IL-23R signaling in Th1 cells that promotes colitis, expanding the importance of this pathway beyond Th17 cells in autoimmune disease and enabling identification of novel targets linked with human IBD for further investigation. The findings provide a reason to target the pathogenic function of IL-23R in autoimmune diseases in which Th1 or other T cell subsets are the main drivers of the disease.

### Pathogenic Th1 Cells

The present invention identified pathogenic Th1 cells. As used herein, pathogenic Th1 cells refer to Th1 cells capable of inducing inflammation or an autoimmune response in vivo. In regard to the digestive track, pathogenic Th1 cells are also referred to herein as colitogenic Th1 cells. In certain embodiments, pathogenic Th1 cells are IFN-γ-producing Th1 cells. In certain embodiments, pathogenic Th1 cells are characterized by high expression of IL-23R or are IL-23R+Th1 cells. In certain embodiments, pathogenic Th1 cells express a gene program comprising one or more of IL23R, CD160, GPR18, GZMB, ITGB1, CCR3, GZMA, IL22, ZFP36L2, ZBTB38, CD74, CCR5, MAP3K8, TNFSF8, IFITM1, NFKBIZ, FOSL2, CREM, CCDC85B, FOS, GPR183, S100A4, 1110008F13RIK, LSP1, LITAF, CD7, DUSP2, PLAC8, H1F0, S1PR1, NCF4, SMIM3, TESC, RBMS1, LPXN, TNFRSF9, PMM1, TOB2, IFNG, CD226, and CTSW.

In certain embodiments, pathogenic Th1 cells express a gene program described herein as cluster 2 or LPL-2 (see, Table 3). For example, the top 50 genes differentially expressed for cluster 2 compared to all other Th1 clusters includes Lgals1, Crip1, S100a6, S100a10, Lgals3, S100a4, Itgb1, Emp3, Actg1, Vim, Ifitm1, Rps27, Rps29, Itgb7, Ms4a4b, Junb, Pglyrp1, Lsp1, Cd52, Klrd1, Got1, Odc1, Nkg7, Cd48, Glipr2, Anxa1, Klf2, Pycard, Rps28, AA467197, Ramp3, Rps27rt, Fxyd5, S1pr4, Rgs1, Bin2, Zyx, Anxa2, Tnfrsf4, Ifitm2, Ctla4, Malat1, Crem, Ifitm3, Myl6, Gnal5, Reep5, Rpl12, S1pr1 and Arhgdib (Positive log FC values are associated with genes with higher expression in cluster 2 and negative log FC values are associated with genes with lower expression in cluster 2). As used herein an increase in cluster 2 means an increase in the gene program that includes both upregulated and downregulated genes.

In certain embodiments, pathogenic Th1 cells express a gene program described herein as cluster 9 or LPL-9 (see, Table 3). For example, the top 50 genes differentially expressed for cluster 9 compared to all other Th1 clusters includes Vps37b, Ubald2, Junb, H3f3b, Ifngr1, Bhlhe40, AW112010, Cd52, Tnfrsf4, Eif1, Odc1, Btg1, Tmsb4x, Gramd3, Hspa5, Ctla4, Jund, Dusp5, Pnrc1, Ifrd1, Prr7, Nfkbia, Pim1, Tnfaip3, Bcl2a1b, Tgif1, Icos, Orail, Il21r, Crem, Cd160, Cytip, Btg2, Rgs2, Gna13, Ramp3, Gpr132, Rsrp1, Hspa8, Kdm6b, Dnaja1, Tbc1d10c, Traf1, Got1, Serpinb9, Tnfsf8, Arf4, Dgat1, Fos12 and Sub1 (Positive log FC values are associated with genes with higher expression in cluster 9 and negative log FC values are associated with genes with lower expression in cluster 9). As used herein an increase in cluster 9 means an increase in the gene program that includes both upregulated and downregulated genes.

In certain embodiments, regulatory Th1 cells express a gene signature or gene program described herein as cluster 7 (see, Table 3). In certain embodiments, regulatory Th1 cells are also referred to as non-pathogenic Th1 cells or Tr1-like cells. For example, the top 50 genes differentially expressed for cluster 7 compared to all other Th1 clusters includes Eomes, Gzmk, Cd27, Furin, Ifitm2, Cxcr6, Cc15, Plac8, Esm1, Gm43291, Sh3bgr13, Tnfsf8, Sh2d1a, Bend4, Rgs16, Tmsb4x, Ly6e, Tmsb10, Rpa2, Ifitm3, Nkg7, Tox, Cd52, Emb, Chchd10, Pim1, Lgals1, Vim, Cldnd1, Rgs10, Actn2, Cxcr5, Lgals3, Capg, Rpl41, AA467197, Tapbpl, Ly6a, Il18r1, Saraf, Asb2, Itgb7, Rasgef1a, Klrg1, Tmem176b, Tnfsf11, Dcp1b, Rps27, Fxyd5 and H2afy (Positive log FC values are associated with genes with higher expression in cluster 7 and negative log FC values are associated with genes with lower expression in cluster 7). As used herein an increase in cluster 7 means an increase in the gene program that includes both upregulated and downregulated genes.

As used herein an increase in a cluster means an increase in the gene program that includes both upregulated and downregulated genes (i.e., there is an increase in Th1 cells expressing the program or the expression of genes in a cell is shifted closer to the gene program than in a control or to any other programs). Clusters as described herein can also be described as a metagene. As used herein a “metagene” refers to a pattern or aggregate of gene expression and not an actual gene. Each metagene may represent a collection or aggregate of genes behaving in a functionally correlated fashion within the genome. The metagene can be increased if the pattern is increased. As used herein the term “gene program” or “program” can be used interchangeably with “biological program”, “expression program”, “transcriptional program”, “expression profile”, “signature”, “gene signature” or “expression program” and may refer to a set of genes that share a role in a biological function (e.g., an activation program, cell differentiation program, proliferation program). Biological programs can include a pattern of gene expression that result in a corresponding physiological event or phenotypic trait (e.g., inflammation). Biological programs can include up to several hundred genes that are expressed in a spatially and temporally controlled fashion. Expression of individual genes can be shared between biological programs. Expression of individual genes can be shared among different single cell types; however, expression of a biological program may be cell type specific or temporally specific (e.g., the biological program is expressed in a cell type at a specific time). Multiple biological programs may include the same gene, reflecting the gene's roles in different processes. Expression of a biological program may be regulated by a master switch, such as a nuclear receptor or transcription factor.

As used herein a “signature” or “gene program” may encompass any gene or genes, protein or proteins, or epigenetic element(s) whose expression profile or whose occurrence is associated with a specific cell type, subtype, or cell state of a specific cell type or subtype within a population of cells. For ease of discussion, when discussing gene expression, any of gene or genes, protein or proteins, or epigenetic element(s) may be substituted. Levels of expression or activity or prevalence may be compared between different cells in order to characterize or identify for instance signatures specific for cell (sub)populations. Increased or decreased expression or activity or prevalence of signature genes may be compared between different cells in order to characterize or identify for instance specific cell (sub)populations. The detection of a signature in single cells may be used to identify and quantitate for instance specific cell (sub)populations. A signature may include a gene or genes, protein or proteins, or epigenetic element(s) whose expression or occurrence is specific to a cell (sub)population, such that expression or occurrence is exclusive to the cell (sub)population. A gene signature as used herein, may thus refer to any set of up- and down-regulated genes that are representative of a cell type or subtype. A gene signature as used herein, may also refer to any set of up- and down-regulated genes between different cells or cell (sub)populations derived from a gene-expression profile. For example, a gene signature may comprise a list of genes differentially expressed in a distinction of interest.

The signature as defined herein (being it a gene signature, protein signature or other genetic or epigenetic signature) can be used to indicate the presence of a cell type, a subtype of the cell type, the state of the microenvironment of a population of cells, a particular cell type population or subpopulation, and/or the overall status of the entire cell (sub)population. Furthermore, the signature may be indicative of cells within a population of cells in vivo. The signature may also be used to suggest for instance particular therapies, or to follow up treatment, or to suggest ways to modulate immune systems. The presence of subtypes or cell states may be determined by subtype specific or cell state specific signatures. The presence of these specific cell (sub)types or cell states may be determined by applying the signature genes to bulk sequencing data in a sample. Not being bound by a theory the signatures of the present invention may be microenvironment specific, such as their expression in a particular spatio-temporal context. Not being bound by a theory, signatures as discussed herein are specific to a particular pathological context. Not being bound by a theory, a combination of cell subtypes having a particular signature may indicate an outcome. Not being bound by a theory, the signatures can be used to deconvolute the network of cells present in a particular pathological condition. Not being bound by a theory the presence of specific cells and cell subtypes are indicative of a particular response to treatment, such as including increased or decreased susceptibility to treatment. The signature may indicate the presence of one particular cell type. In one embodiment, the novel signatures are used to detect multiple cell states or hierarchies that occur in subpopulations of immune cells that are linked to particular pathological condition (e.g., inflammation), or linked to a particular outcome or progression of the disease (e.g., autoimmunity), or linked to a particular response to treatment of the disease.

The signature according to certain embodiments of the present invention may comprise or consist of one or more genes, proteins and/or epigenetic elements, such as for instance 1, 2, 3, 4, 5, 6, 7, 8, 9, 10 or more. In certain embodiments, the signature may comprise or consist of two or more genes, proteins and/or epigenetic elements, such as for instance 2, 3, 4, 5, 6, 7, 8, 9, 10 or more. In certain embodiments, the signature may comprise or consist of three or more genes, proteins and/or epigenetic elements, such as for instance 3, 4, 5, 6, 7, 8, 9, 10 or more. In certain embodiments, the signature may comprise or consist of four or more genes, proteins and/or epigenetic elements, such as for instance 4, 5, 6, 7, 8, 9, 10 or more. In certain embodiments, the signature may comprise or consist of five or more genes, proteins and/or epigenetic elements, such as for instance 5, 6, 7, 8, 9, 10 or more. In certain embodiments, the signature may comprise or consist of six or more genes, proteins and/or epigenetic elements, such as for instance 6, 7, 8, 9, 10 or more. In certain embodiments, the signature may comprise or consist of seven or more genes, proteins and/or epigenetic elements, such as for instance 7, 8, 9, 10 or more. In certain embodiments, the signature may comprise or consist of eight or more genes, proteins and/or epigenetic elements, such as for instance 8, 9, 10 or more. In certain embodiments, the signature may comprise or consist of nine or more genes, proteins and/or epigenetic elements, such as for instance 9, 10 or more. In certain embodiments, the signature may comprise or consist of ten or more genes, proteins and/or epigenetic elements, such as for instance 10, 11, 12, 13, 14, 15, or more. It is to be understood that a signature according to the invention may for instance also include genes or proteins as well as epigenetic elements combined.

It is to be understood that “differentially expressed” genes/proteins include genes/proteins which are up- or down-regulated as well as genes/proteins which are turned on or off. When referring to up-or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art.

As discussed herein, differentially expressed genes/proteins, or differential epigenetic elements may be differentially expressed on a single cell level, or may be differentially expressed on a cell population level. Preferably, the differentially expressed genes/proteins or epigenetic elements as discussed herein, such as constituting the gene signatures as discussed herein, when as to the cell population level, refer to genes that are differentially expressed in all or substantially all cells of the population (such as at least 80%, preferably at least 90%, such as at least 95% of the individual cells). This allows one to define a particular subpopulation of tumor cells. As referred to herein, a “subpopulation” of cells preferably refers to a particular subset of cells of a particular cell type which can be distinguished or are uniquely identifiable and set apart from other cells of this cell type. The cell subpopulation may be phenotypically characterized, and is preferably characterized by the signature as discussed herein. A cell (sub)population as referred to herein may constitute of a (sub)population of cells of a particular cell type characterized by a specific cell state.

When referring to induction, or alternatively suppression of a particular signature, preferable is meant induction or alternatively suppression (or upregulation or downregulation) of at least one gene/protein and/or epigenetic element of the signature, such as for instance at least two, at least three, at least four, at least five, at least six, or all genes/proteins and/or epigenetic elements of the signature.

As used herein, all gene name symbols refer to the gene as commonly known in the art. The examples described herein that refer to the mouse gene names are to be understood to also encompasses human genes, as well as genes in any other organism (e.g., homologous, orthologous genes). Mouse gene symbols are generally italicized, with only the first letter in upper-case (e.g., 1123). Mouse protein symbols are generally not italicized, and all letters are in upper-case (e.g., IL-23). As used herein mouse gene symbols may be shown with only the first letter in upper-case and not italicized (e.g., 11123). Any reference to the gene symbol is a reference made to the entire gene or variants of the gene. Any reference to the gene symbol is also a reference made to the gene product (e.g., protein). The term, homolog, may apply to the relationship between genes separated by the event of speciation (e.g., ortholog). Orthologs are genes in different species that evolved from a common ancestral gene by speciation. Normally, orthologs retain the same function in the course of evolution. Gene symbols may be those referred to by the HUGO Gene Nomenclature Committee (HGNC) or National Center for Biotechnology Information (NCBI). The signature as described herein may encompass any of the genes described herein.

### Functional T Cell Immune States

The present invention relates to immune cell balance and function, in particular Th1 cell pathogenicity or non-pathogenicty. In general, aberrant immune balance can lead to autoimmunity when the immune response is too strong and cancer when the immune response is suppressed. An immune response can include the interaction of several immune cell types and different immune responses can be mediated by specific immune cells. In certain embodiments, T cells can affect the overall immune state, such as other immune cells in proximity.

The term “immune cell” as used throughout this specification generally encompasses any cell derived from a hematopoietic stem cell that plays a role in the immune response. The term is intended to encompass immune cells both of the innate or adaptive immune system. The immune cell as referred to herein may be a leukocyte, at any stage of differentiation (e.g., a stem cell, a progenitor cell, a mature cell) or any activation stage. Immune cells include lymphocytes (such as natural killer cells, T-cells (including, e.g., thymocytes, Th or Tc; Th1, Th2, Th17, Thαβ, CD4+, CD8+, effector Th, memory Th, regulatory Th, CD4+/CD8+ thymocytes, CD4−/CD8− thymocytes, γδ T cells, etc.) or B-cells (including, e.g., pro-B cells, early pro-B cells, late pro-B cells, pre-B cells, large pre-B cells, small pre-B cells, immature or mature B-cells, producing antibodies of any isotype, T1 B-cells, T2, B-cells, naïve B-cells, GC B-cells, plasmablasts, memory B-cells, plasma cells, follicular B-cells, marginal zone B-cells, B-1 cells, B-2 cells, regulatory B cells, etc.), such as for instance, monocytes (including, e.g., classical, non-classical, or intermediate monocytes), (segmented or banded) neutrophils, eosinophils, basophils, mast cells, histiocytes, microglia, including various subtypes, maturation, differentiation, or activation stages, such as for instance hematopoietic stem cells, myeloid progenitors, lymphoid progenitors, myeloblasts, promyelocytes, myelocytes, metamyelocytes, monoblasts, promonocytes, lymphoblasts, prolymphocytes, small lymphocytes, macrophages (including, e.g., Kupffer cells, stellate macrophages, M1 or M2 macrophages), (myeloid or lymphoid) dendritic cells (including, e.g., Langerhans cells, conventional or myeloid dendritic cells, plasmacytoid dendritic cells, mDC-1, mDC-2, Mo-DC, HP-DC, veiled cells), granulocytes, polymorphonuclear cells, antigen-presenting cells (APC), etc.

As used throughout this specification, “immune response” refers to a response by a cell of the immune system, such as a B cell, T cell (CD4+ or CD8+), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”), and refers to a response by a CD4 T cell, CD8 T cell, or B cell via their antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response.

T cell response refers more specifically to an immune response in which T cells directly or indirectly mediate or otherwise contribute to an immune response in a subject. T cell-mediated response may be associated with cell mediated effects, cytokine mediated effects, and even effects associated with B cells if the B cells are stimulated, for example, by cytokines secreted by T cells. By means of an example but without limitation, effector functions of NMC class I restricted Cytotoxic T lymphocytes (CTLs), may include cytokine and/or cytolytic capabilities, such as lysis of target cells presenting an antigen peptide recognized by the T cell receptor (naturally-occurring TCR or genetically engineered TCR, e.g., chimeric antigen receptor, CAR), secretion of cytokines, preferably IFN gamma, TNF alpha and/or or more immunostimulatory cytokines, such as IL-2, and/or antigen peptide-induced secretion of cytotoxic effector molecules, such as granzymes, perforins or granulysin. By means of example but without limitation, for NMC class II restricted T helper (Th) cells, effector functions may be antigen peptide-induced secretion of cytokines, preferably, IFN gamma, TNF alpha, IL-4, IL5, IL-10, and/or IL-2. By means of example but without limitation, for T regulatory (Treg) cells, effector functions may be antigen peptide-induced secretion of cytokines, preferably, IL-10, IL-35, and/or TGF-beta. B cell response refers more specifically to an immune response in which B cells directly or indirectly mediate or otherwise contribute to an immune response in a subject. Effector functions of B cells may include in particular production and secretion of antigen-specific antibodies by B cells (e.g., polyclonal B cell response to a plurality of the epitopes of an antigen (antigen-specific antibody response)), antigen presentation, and/or cytokine secretion.

During persistent immune activation, such as during uncontrolled tumor growth or chronic infections, subpopulations of immune cells, particularly of CD8+ or CD4+ T cells, become compromised to different extents with respect to their cytokine and/or cytolytic capabilities. Such immune cells, particularly CD8+ or CD4+ T cells, are commonly referred to as “dysfunctional” or as “functionally exhausted” or “exhausted”. As used herein, the term “dysfunctional” or “functional exhaustion” refer to a state of a cell where the cell does not perform its usual function or activity in response to normal input signals, and includes refractivity of immune cells to stimulation, such as stimulation via an activating receptor or a cytokine. Such a function or activity includes, but is not limited to, proliferation (e.g., in response to a cytokine, such as IFN-gamma) or cell division, entrance into the cell cycle, cytokine production, cytotoxicity, migration and trafficking, phagocytotic activity, or any combination thereof. Normal input signals can include, but are not limited to, stimulation via a receptor (e.g., T cell receptor, B cell receptor, co-stimulatory receptor). Unresponsive immune cells can have a reduction of at least 10%, 20%, 30%, 40%, 50%, 60%, 70%, 80%, 90%, 95%, or even 100% in cytotoxic activity, cytokine production, proliferation, trafficking, phagocytotic activity, or any combination thereof, relative to a corresponding control immune cell of the same type. In some particular embodiments of the aspects described herein, a cell that is dysfunctional is a CD8+ T cell that expresses the CD8+ cell surface marker. Such CD8+ cells normally proliferate and produce cell killing enzymes, e.g., they can release the cytotoxins perforin, granzymes, and granulysin. However, exhausted/dysfunctional T cells do not respond adequately to TCR stimulation, and display poor effector function, sustained expression of inhibitory receptors and a transcriptional state distinct from that of functional effector or memory T cells. Dysfunction/exhaustion of T cells thus prevents optimal control of infection and tumors. Exhausted/dysfunctional immune cells, such as T cells, such as CD8+ T cells, may produce reduced amounts of IFN-gamma, TNF-alpha and/or one or more immunostimulatory cytokines, such as IL-2, compared to functional immune cells. Exhausted/dysfunctional immune cells, such as T cells, such as CD8+ T cells, may further produce (increased amounts of) one or more immunosuppressive transcription factors or cytokines, such as IL-10 and/or Foxp3, compared to functional immune cells, thereby contributing to local immunosuppression. Dysfunctional CD8+ T cells can be both protective and detrimental against disease control. As used herein, a “dysfunctional immune state” refers to an overall suppressive immune state in a subject or microenvironment of the subject (e.g., tumor microenvironment). For example, increased IL-10 production leads to suppression of other immune cells in a population of immune cells.

CD8+ T cell function is associated with their cytokine profiles. It has been reported that effector CD8+ T cells with the ability to simultaneously produce multiple cytokines (polyfunctional CD8+ T cells) are associated with protective immunity in patients with controlled chronic viral infections as well as cancer patients responsive to immune therapy (Spranger et al., 2014, J. Immunother. Cancer, vol. 2, 3). In the presence of persistent antigen CD8+ T cells were found to have lost cytolytic activity completely over time (Moskophidis et al., 1993, Nature, vol. 362, 758-761). It was subsequently found that dysfunctional T cells can differentially produce IL-2, TNFa and IFNg in a hierarchical order (Wherry et al., 2003, J. Virol., vol. 77, 4911-4927). Decoupled dysfunctional and activated CD8+ cell states have also been described (see, e.g., Singer, et al. (2016). A Distinct Gene Module for Dysfunction Uncoupled from Activation in Tumor-Infiltrating T Cells. Cell 166, 1500-1511 e1509; WO/2017/075478; and WO/2018/049025).

The invention provides compositions and methods for modulating T cell balance, in particular Th1 cell pathogenicity (i.e., balance between pathogenic and non-pathogenic Th1 cells). The invention provides T cell modulating agents that modulate T cell balance. For example, in some embodiments, the invention provides T cell modulating agents and methods of using these T cell modulating agents to regulate, influence or otherwise impact the level of and/or balance of Th1 cells. For example, in some embodiments, the invention provides T cell modulating agents and methods of using these T cell modulating agents to regulate, influence or otherwise impact the level of and/or balance between Th1 activity and inflammatory potential. As used herein, terms such as “Th1 cell” and/or “Th1 phenotype” and all grammatical variations thereof refer to a differentiated T helper cell that expresses interferon gamma (IFNγ). Further, Th1 cells are characterized as expressing IFN-γ, IL-2, lymphotoxin (LT) and the master transcription factor T-bet but do not produce IL-17A, the signature cytokine of Th17 cells.

In certain embodiments, Th17 balance is modified in combination with Th1 cells. As used herein, terms such as “Th17 cell” and/or “Th17 phenotype” and all grammatical variations thereof refer to a differentiated T helper cell that expresses one or more cytokines selected from the group the consisting of interleukin 17A (IL-17A), interleukin 17F (IL-17F), and interleukin 17A/F heterodimer (IL17-AF). As used herein, terms such as “Th2 cell” and/or “Th2 phenotype” and all grammatical variations thereof refer to a differentiated T helper cell that expresses one or more cytokines selected from the group the consisting of interleukin 4 (IL-4), interleukin 5 (IL-5) and interleukin 13 (IL-13). As used herein, terms such as “Treg cell” and/or “Treg phenotype” and all grammatical variations thereof refer to a differentiated T cell that expresses Foxp3.

Depending on the cytokines used for differentiation, in vitro polarized Th17 cells can either cause severe autoimmune responses upon adoptive transfer (‘pathogenic Th17 cells’) or have little or no effect in inducing autoimmune disease (‘non-pathogenic cells’) (Ghoreschi et al., 2010; and Lee et al., 2012 “Induction and molecular signature of pathogenic Th17 cells,” Nature Immunology, vol. 13(10): 991-999). In vitro differentiation of naïve CD4 T cells in the presence of TGF-β1+IL-6 induces an IL-17A and IL-10 producing population of Th17 cells, that are generally nonpathogenic, whereas activation of naïve T cells in the presence of IL-1β+IL-6+IL-23 or TGF-β3+IL-6 induces a T cell population that produces IL-17A and IFN-γ, and are potent inducers of autoimmune disease induction (Ghoreschi et al., 2010, Lee et al., 2012).

As used herein, terms such as “pathogenic Th17 cell” and/or “pathogenic Th17 phenotype” and all grammatical variations thereof refer to Th17 cells that, exhibit a distinct pathogenic signature where one or more of the following genes or products of these genes is upregulated in Th17 cells: Cxcl3, Il22, Il3, Ccl4, Gzmb, Lrmp, Cc15, Casp1, Csf2, Ccl3, Tbx21, Icos, Il7r, Stat4, Lgals3 or Lag3. In certain embodiments, the pathogenic signature is elevated in TGF-β3-induced Th17 cells as compared to TGF-β1-induced Th17 cells. As used herein, terms such as “non-pathogenic Th17 cell” and/or “non-pathogenic Th17 phenotype” and all grammatical variations thereof refer to Th17 cells that exhibit a distinct non-pathogenic signature where one or more of the following genes or products of these genes is up-regulated in Th17 cells: Il6st, Il1rn, lkzf3, Maf, Ahr, Il9 or Il10. In certain embodiments, the non-pathogenic signature is elevated in TGF-β1-induced Th17 cells as compared to TGF-β3-induced Th17 cells. In certain embodiments, when induced in the presence of TGF-β3, Th17 cells express a decreased level of one or more genes selected from IL6st, IL1rn, Ikzf3, Maf, Ahr, IL9 and IL10, as compared to the level of expression in TGF-β3-induced Th17 cells.

A dynamic regulatory network controls Th17 differentiation (See e.g., Yosef et al., Dynamic regulatory network controlling Th17 cell differentiation, Nature, vol. 496: 461-468 (2013); Wang et al., CD5L/AIM Regulates Lipid Biosynthesis and Restrains Th17 Cell Pathogenicity, Cell Volume 163, Issue 6, p 1413-1427, 3 Dec. 2015; Gaublomme et al., Single-Cell Genomics Unveils Critical Regulators of Th17 Cell Pathogenicity, Cell Volume 163, Issue 6, p 1400-1412, 3 Dec. 2015; and International publication numbers WO2016138488A2, WO2015130968, WO/2012/048265, WO/2014/145631 and WO/2014/134351, the contents of which are hereby incorporated by reference in their entirety).

### Diseases

In certain embodiments, diseases or conditions are treated, monitored or detected. In certain embodiments, diseases relevant to the present invention are caused by pathogenic Th1 cells or are capable of being controlled by pathogenic Th1 cells. In certain embodiments, the diseases are associated with IL-23 or IL-23R.

In certain embodiments, the diseases are associated with IL-23R by genome wide association studies (GWAS) (e.g., Duerr R H, Taylor K D, Brant S R, et al. A genome-wide association study identifies TL23R as an inflammatory bowel disease gene. Science. 2006; 314(5804):1461-1463; Zhu W, Deng Y, Zhou X. Multiple Membrane Transporters and Some Immune Regulatory Genes are Major Genetic Factors to Gout. Open Rheumatol J. 2018; 12:94-113; Peng L L, Wang Y, Zhu F L, Xu W D, Ji X L, Ni J. IL-23R mutation is associated with ulcerative colitis: A systemic review and meta-analysis. Oncotarget. 2017; 8(3):4849-4863; Zhang F, Liu H, Chen S, et al. Identification of two new loci at IL23R and RAB32 that influence susceptibility to leprosy. Nat Genet. 2011; 43(12):1247-1251; Mizuki N, Meguro A, Ota M, et al. Genome-wide association studies identify IL23R-IL12RB2 and IL10 as Behçet's disease susceptibility loci. Nat Genet. 2010; 42(8):703-706; and Remmers E F, Cosan F, Kirino Y, et al. Genome-wide association study identifies variants in the MHC class I, IL10, and IL23R-IL12RB2 regions associated with Behçet's disease. Nat Genet. 2010; 42(8):698-702). For example, shifting Th1 cell pathogenicity may be used to treat ankylosing spondylitis, psoriasis, ulcerative colitis, Crohn's disease, sclerosing cholangitis, inflammatory bowel disease (IBD), adolescent idiopathic scoliosis, autoimmune thyroid disease, type I diabetes mellitus, Common variable immunodeficiency, celiac disease, juvenile idiopathic arthritis, systemic lupus erythematosus, malaria, psoriatic arthritis, anterior uveitis, type II diabetes mellitus, psoriasis vulgaris or tuberculosis (see, e.g., Buniello, et al. The NHGRI-EBI GWAS Catalog of published genome-wide association studies, targeted arrays and summary statistics 2019. Nucleic Acids Research, 2019, Vol. 47 (Database issue): D1005-D1012).

In certain embodiments, autoimmune or inflammatory diseases are treated, monitored or detected. As used throughout the present specification, the terms “autoimmune disease” or “autoimmune disorder” used interchangeably refer to a diseases or disorders caused by an immune response against a self-tissue or tissue component (self-antigen) and include a self-antibody response and/or cell-mediated response. The terms encompass organ-specific autoimmune diseases, in which an autoimmune response is directed against a single tissue, as well as non-organ specific autoimmune diseases, in which an autoimmune response is directed against a component present in two or more, several or many organs throughout the body. Examples of autoimmune diseases include but are not limited to acute disseminated encephalomyelitis (ADEM); Addison's disease; ankylosing spondylitis; antiphospholipid antibody syndrome (APS); aplastic anemia; autoimmune gastritis; autoimmune hepatitis; autoimmune thrombocytopenia; Behçet's disease; coeliac disease; dermatomyositis; diabetes mellitus type I; Goodpasture's syndrome; Graves' disease; Guillain-Barre syndrome (GBS); Hashimoto's disease; idiopathic thrombocytopenic purpura; inflammatory bowel disease (IBD) including Crohn's disease and ulcerative colitis; mixed connective tissue disease; multiple sclerosis (MS); myasthenia gravis; opsoclonus myoclonus syndrome (OMS); optic neuritis; Ord's thyroiditis; pemphigus; pernicious anaemia; polyarteritis nodosa; polymyositis; primary biliary cirrhosis; primary myoxedema; psoriasis; rheumatic fever; rheumatoid arthritis; Reiter's syndrome; scleroderma; Sjögren's syndrome; systemic lupus erythematosus; Takayasu's arteritis; temporal arteritis; vitiligo; warm autoimmune hemolytic anemia; or Wegener's granulomatosis.

Examples of inflammatory diseases or disorders include, but are not limited to, asthma, allergy, allergic rhinitis, allergic airway inflammation, atopic dermatitis (AD), chronic obstructive pulmonary disease (COPD), inflammatory bowel disease (IBD), Irritable bowel syndrome (IBS), multiple sclerosis, arthritis, psoriasis, eosinophilic esophagitis, eosinophilic pneumonia, eosinophilic psoriasis, hypereosinophilic syndrome, graft-versus-host disease, uveitis, cardiovascular disease, pain, multiple sclerosis, lupus, vasculitis, chronic idiopathic urticaria and Eosinophilic Granulomatosis with Polyangiitis (Churg-Strauss Syndrome).

In certain embodiments, cancer is treated by increasing Th1 pathogenicity or a cancer treatment is monitored by detecting Th1 pathogenicity. The cancer may include, without limitation, liquid tumors such as leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, or multiple myeloma.

The cancer may include, without limitation, solid tumors such as sarcomas and carcinomas. Examples of solid tumors include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, epithelial carcinoma, bronchogenic carcinoma, hepatoma, colorectal cancer (e.g., colon cancer, rectal cancer), anal cancer, pancreatic cancer (e.g., pancreatic adenocarcinoma, islet cell carcinoma, neuroendocrine tumors), breast cancer (e.g., ductal carcinoma, lobular carcinoma, inflammatory breast cancer, clear cell carcinoma, mucinous carcinoma), ovarian carcinoma (e.g., ovarian epithelial carcinoma or surface epithelial-stromal tumour including serous tumour, endometrioid tumor and mucinous cystadenocarcinoma, sex-cord-stromal tumor), prostate cancer, liver and bile duct carcinoma (e.g., hepatocelluar carcinoma, cholangiocarcinoma, hemangioma), choriocarcinoma, seminoma, embryonal carcinoma, kidney cancer (e.g., renal cell carcinoma, clear cell carcinoma, Wilm's tumor, nephroblastoma), cervical cancer, uterine cancer (e.g., endometrial adenocarcinoma, uterine papillary serous carcinoma, uterine clear-cell carcinoma, uterine sarcomas and leiomyosarcomas, mixed mullerian tumors), testicular cancer, germ cell tumor, lung cancer (e.g., lung adenocarcinoma, squamous cell carcinoma, large cell carcinoma, bronchioloalveolar carcinoma, non-small-cell carcinoma, small cell carcinoma, mesothelioma), bladder carcinoma, signet ring cell carcinoma, cancer of the head and neck (e.g., squamous cell carcinomas), esophageal carcinoma (e.g., esophageal adenocarcinoma), tumors of the brain (e.g., glioma, glioblastoma, astrocytoma, medulloblastoma, craniopharyngioma, ependymoma, pinealoma, hemangioblastoma, acoustic neuroma, oligodenroglioma, schwannoma, meningioma), neuroblastoma, retinoblastoma, neuroendocrine tumor, melanoma, cancer of the stomach (e.g., stomach adenocarcinoma, gastrointestinal stromal tumor), or carcinoids. Lymphoproliferative disorders are also considered to be proliferative diseases.

### In Vitro Differentiated Pathogenic Th1 Cells

In certain embodiments, pathogenic Th1 cells can be obtained by differentiating naïve CD4+ T cells in vitro. The naïve T cells may be obtained from a healthy donor subject. The naïve T cells may be obtained from a subject suffering from an aberrant immune response. The naïve T cells may be obtained from a subject suffering from cancer. The naïve T cells may be obtained from an animal model, such as a mouse model. In certain embodiments, the mouse model is a model of any disease described herein. T cells may be obtained from the spleen or lymph nodes. The T cells may be obtained from a blood sample. CD4⁺ T cells can be enriched using any method commonly used in the art, such as microbeads. Naïve CD4⁺ T cells can be isolated using any method known in the art, such as cell sorting. In certain embodiments, naïve CD4+ T cells (CD4⁺CD44^{low}CD62L^{high}CD25⁻) can be isolated by fluorescence-activated cell sorting (FACS). In certain embodiments, naïve CD4+ T cells are activated (e.g., with anti-CD3 and anti-CD28 antibodies). In certain embodiments, naïve CD4+ T cells are differentiated with cytokines. In certain embodiments, pathogenic Th1 cells are obtained by differentiating with IL-12 and IL-21. In certain embodiments, pathogenic Th1 cells are obtained by differentiating with IL-12, IL-21 and IL-23. Recombinant cytokines are commercially available.

The differentiated pathogenic Th1 cells may be used for various applications described further herein. For example, in methods of screening for agents capable of shifting Th1 pathogenicity or for adoptive transfer. In certain embodiments, adoptively transferred pathogenic Th1 cells can be used to enhance anti-tumor immunity. In certain embodiments, the Th1 cells are specific for a tumor antigen as described herein.

### Therapeutic Methods

In certain embodiments, shifting Th1 pathogenicity can be used in a therapeutic method (i.e., treatment) to decrease or increase an immune response. In certain embodiments, autoimmune diseases are treated by specifically targeting pathogenic Th1 cells or decreasing pathogenic Th1 cells. In certain embodiments, cancer is treated by specifically targeting Th1 cells or increasing pathogenic Th1 cells. Targeting Th1 cells can be performed by targeting specific pathogenic Th1 cell programs or markers, as described herein. In certain embodiments, surface markers are targeted. In certain embodiments, CD160 expression or activity is inhibited to decrease Th1 pathogenicity. In certain embodiments, CD160 expression or activity is enhanced or activated to increase Th1 pathogenicity. In certain embodiments, one or markers selected from CD160, GPR18, GZMB, ITGB1, CCR3, GZMA, IL22, ZFP36L2, ZBTB38, CD74, CCR5, MAP3K8, TNFSF8, IFITM1, NFKBIZ, FOSL2, CREM, CCDC85B, FOS, GPR183, S100A4, 1110008F13RIK, LSP1, LITAF, CD7, DUSP2, PLAC8, H1F0, S1PR1, NCF4, SMIM3, TESC, RBMS1, LPXN, TNFRSF9, PMM1, TOB2, IFNG, CD226, and CTSW is targeted by one or more therapeutic agents. In certain embodiments, Th1 cells are targeted with one or more therapeutic agents. In certain embodiments, small molecules may be used to inhibit a target that has enzymatic activity. In certain embodiments, binding molecules, such as antibodies, are used to target surface proteins.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. As used herein “treating” includes ameliorating, curing, preventing it from becoming worse, slowing the rate of progression, or preventing the disorder from re-occurring (i.e., to prevent a relapse).

In certain embodiments, diabetes is treated. Type 1 diabetes is an autoimmune disease where the insulin-producing beta cells in the pancreas are wrongly detected as foreign and destroyed by the immune system (e.g., pathogenic Th1 cells). In certain embodiments, Type I diabetes can be treated at two main stages of the disease process—prior to clinical onset but after the appearance of islet autoantibodies (secondary prevention) and immediately after diagnosis (intervention) (see, e.g., Staeva-Vieira T, Peakman M, von Herrath M. Translational mini-review series on type 1 diabetes: Immune-based therapeutic approaches for type 1 diabetes [published correction appears in Clin Exp Immunol. 2007 July; 149(1):203]. Clin Exp Immunol. 2007; 148(1):17-31). At the intervention stage there is still residual beta cell mass (about 15-20%). Id.

In certain embodiments, IBD is treated by targeting pathogenic Th1 cells. Inflammatory bowel disease (IBD) is a chronic disabling inflammatory process that affects mainly the gastrointestinal tract and may present associated extraintestinal manifestations (see, e.g., Catalan-Serra I, Brenna Ø. Immunotherapy in inflammatory bowel disease: Novel and emerging treatments. Hum Vaccin Immunother. 2018; 14(11):2597-2611). IBD includes both ulcerative colitis (UC) and Crohn's disease (CD). Id. The IL-23 pathway is altered in both conditions. Id Current pharmacological treatments used in clinical practice like thiopurines or anti-TNF are effective but can produce significant side effects and their efficacy may diminish over time. Id. The current treatment of IBD includes mesalazine (oral and rectal formulations), glucocorticoids (conventional and other forms like budesonide or beclomethasone), antibiotics (typically ciprofloxacine and metronidazole), immunosuppressants (mostly azathioprine/6-mercaptopurine or methotrexate) and anti-TNF agents (infliximab, adalimumab, certolizumab pegol and golimumab). Recently, the anti-integrin antibody vedolizumab and the antibody against IL-12/23 ustekinumab have been approved for IBD. Id. In certain embodiments, targeting pathogenic Th1 cells can be used to control inflammation in IBD without producing significant side effects. In certain embodiments, the treatment as described herein is administered in combination with the current treatments, such that side effects are diminished. In certain embodiments, the standard doses can be decreased in such combination treatments.

In certain embodiments, the present invention provides for one or more therapeutic agents against one or more of the targets identified. In certain embodiments, the one or more agents comprises a small molecule inhibitor, small molecule degrader (e.g., ATTEC, AUTAC, LYTAC, or PROTAC), genetic modifying agent, antibody, antibody fragment, antibody-like protein scaffold, aptamer, protein, or any combination thereof.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

In certain embodiments, the therapeutic agents are administered in an effective amount or therapeutically effective amount. The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

In certain embodiments, an agent against one of the targets is used in combination with a treatment already be known or used clinically. In certain embodiments, targeting the combination may require less of the agent as compared to the current standard of care and provide for less toxicity and improved treatment. The term “standard of care” as used herein refers to the current treatment that is accepted by medical experts as a proper treatment for a certain type of disease and that is widely used by healthcare professionals. Standard of care is also called best practice, standard medical care, and standard therapy.

In certain embodiments, the agents are used to modulate cell types in vivo. In certain embodiments, the agents are used to modulate cell types in vitro. For example, the agents may be used to modulate cells for adoptive cell transfer.

### CD160

In certain embodiments, CD160 is targeted for shifting Th1 cell pathogenicity. In certain embodiments, inhibiting CD160 reduces Th1 pathogenicity and enhancing CD160 increases Th1 pathogenicity. As used herein CD160 (also known as, BY55, NK1, NK28) refers to the CD160 molecule. Exemplary sequences include NCBI accession numbers NM_007053.4 and NP_008984.1. CD160 is a glycoprotein and ligand for Herpesvirus entry mediator (HVEM), also known as tumor necrosis factor receptor superfamily member 14 (TNFRSF14), and considered a proposed immune checkpoint inhibitor with anti-cancer activity along with anti-PD-1 antibodies (Stecher C, Battin C, Leitner J, et al. PD-1 Blockade Promotes Emerging Checkpoint Inhibitors in Enhancing T Cell Responses to Allogeneic Dendritic Cells. Front Immunol. 2017; 8:572). Blocking checkpoint inhibitors enhances an immune response. The present invention shows for the first time that CD160 is necessary for Th1 cell pathogenicity and inhibiting CD160 decreases an immune response. Stecher et al. discloses monoclonal-blocking antibodies for CD160 that can be used as a therapeutic to decrease inflammation or autoimmunity caused by pathogenic Th1 cells. Other non-limiting CD160 antibodies applicable to the present invention have been described (Menguy T, Briaux A, Jeunesse E, et al. Anti-CD160, Alone or in Combination With Bevacizumab, Is a Potent Inhibitor of Ocular Neovascularization in Rabbit and Monkey Models. Invest Ophthalmol Vis Sci. 2018; 59(7):2687-2698; El-Far M, Pellerin C, Pilote L, et al. CD160 isoforms and regulation of CD4 and CD8 T-cell responses. J Transl Med. 2014; 12:217; U.S. Ser. No. 10/208,124B2; US20200109211A1; US20190256595A1; WO2010084158A1; and US20150004170A1).

### GPR18

In certain embodiments, GPR18 is targeted for shifting Th1 cell pathogenicity. In certain embodiments, inhibiting GPR18 reduces Th1 pathogenicity and enhancing GPR18 increases Th1 pathogenicity. As used herein GPR18 refers to the G protein-coupled receptor 18 (also known as, N-Arachidonyl glycine receptor, NAGly receptor). Exemplary sequences include NCBI accession numbers NM_001098200.2, NM_005292.4, NP 001091670.1 and NP_005283.1. Non-limiting GPR18 agonists include PSB-KK-1415, PSB-KD107, PSB-KD477, N-Arachidonoylglycine (NAGly), Abnormal cannabidiol (Abn-CBD), AM-251, Cannabidiol, O-1602, Δ⁹-Tetrahydrocannabinol (Δ⁹-THC), Anandamide (N-arachidonoyl ethanolamine, AEA), Arachidonylcyclopropylamide (ACPA), and Resolvin D2 (RvD2). Non-limiting GPR18 antagonists include PSB-CB5, Amauromine, O-1918, and CID-85469571. See, also, Fabisiak A, Fabisiak N, Mokrowiecka A, et al. Novel selective agonist of GPR18, PSB-KK-1415 exerts potent anti-inflammatory and anti-nociceptive activities in animal models of intestinal inflammation and inflammatory pain [published online ahead of print, 2020 Oct. 14]. Neurogastroenterol Motil. 2020; e14003; and Schoeder C T, Mahardhika A B, Drabczynska A, Kied-Kononowicz K, Müller CE. Discovery of Tricyclic Xanthines as Agonists of the Cannabinoid-Activated Orphan G-Protein-Coupled Receptor GPR18. ACS Med Chem Lett. 2020; 11(10):2024-2031.

### Antibodies

In certain embodiments, the one or more agent is an antibody. In certain embodiments, an antibody targets one or more surface genes or polypeptides. The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, V_{HH} and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g., the IgG1, IgG2, IgG3, and IgG4 subclassess of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, lgM antibodies exist in pentameric form, and IgA antibodies exist in monomeric, dimeric or multimeric form.

The term “IgG subclass” refers to the four subclasses of immunoglobulin class IgG-IgG1, IgG2, IgG3, and IgG4 that have been identified in humans and higher mammals by the heavy chains of the immunoglobulins, VI—7⁴, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by p pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g., LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (1° F.n3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins-harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, SnM or less, lnM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the CHI domain; (v) the Fv fragment having the V_{L} and V_{H} domains of a single arm of an antibody; (vi) the dAb fragment (Ward et al., 341 Nature 544 (1989)) which consists of a V_{H} domain or a V_{L} domain that binds antigen; (vii) isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}-Cₕ1-V_{H}-Cₕ1) which, together with complementary light chain polypeptides, form a pair of antigen binding regions (Zapata et al., Protein Eng. 8(10):1057-62 (1995); and U.S. Pat. No. 5,641,870).

As used herein, a “blocking” antibody or an antibody “antagonist” is one which inhibits or reduces biological activity of the antigen(s) it binds (e.g., CD160). In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect agents that bind to a target protein, or disrupt the interaction between proteins (e.g., a receptor and a ligand). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene signature as described herein.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

### Bispecific Antibodies

In certain embodiments, bispecific antibodies are used to target pathogenic Th1 cells. Bi-specific antigen-binding constructs, e.g., bi-specific antibodies (bsAb) or BiTEs, bind two antigens (see, e.g., Suurs et al., A review of bispecific antibodies and antibody constructs in oncology and clinical challenges. Pharmacol Ther. 2019 September; 201:103-119; and Huehls, et al., Bispecific T cell engagers for cancer immunotherapy. Immunol Cell Biol. 2015 March; 93(3): 290-296). The bi-specific antigen-binding construct includes two antigen-binding polypeptide constructs, e.g., antigen binding domains. In some embodiments, the antigen-binding construct is derived from known antibodies or antigen-binding constructs. In some embodiments, the antigen-binding polypeptide constructs comprise two antigen binding domains that comprise antibody fragments. In some embodiments, the first antigen binding domain and second antigen binding domain each independently comprises an antibody fragment selected from the group of: an scFv, a Fab, and an Fc domain. The antibody fragments may be the same format or different formats from each other. For example, in some embodiments, the antigen-binding polypeptide constructs comprise a first antigen binding domain comprising an scFv and a second antigen binding domain comprising a Fab. In some embodiments, the antigen-binding polypeptide constructs comprise a first antigen binding domain and a second antigen binding domain, wherein both antigen binding domains comprise an scFv. In some embodiments, the first and second antigen binding domains each comprise a Fab. In some embodiments, the first and second antigen binding domains each comprise an Fc domain. Any combination of antibody formats is suitable for the bi-specific antibody constructs disclosed herein.

In certain embodiments, Th1 cells can be engaged to tumor cells. In certain embodiments, two targets are disrupted on a Th1 cell by the bsAb. By means of an example, an agent, such as a bi-specific antibody, capable of specifically binding to a gene product expressed on the cell surface of the Th1 cells and a tumor cell may be used for targeting Th1 cells to tumor cells.

### Aptamers

In certain embodiments, the one or more agent is an aptamer. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, cells, tissues and organisms. Nucleic acid aptamers have specific binding affinity to molecules through interactions other than classic Watson-Crick base pairing. Aptamers are useful in biotechnological and therapeutic applications as they offer molecular recognition properties similar to antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. In certain embodiments, RNA aptamers may be expressed from a DNA construct. In other embodiments, a nucleic acid aptamer may be linked to another polynucleotide sequence. The polynucleotide sequence may be a double stranded DNA polynucleotide sequence. The aptamer may be covalently linked to one strand of the polynucleotide sequence. The aptamer may be ligated to the polynucleotide sequence. The polynucleotide sequence may be configured, such that the polynucleotide sequence may be linked to a solid support or ligated to another polynucleotide sequence.

Aptamers, like peptides generated by phage display or monoclonal antibodies (“mAbs”), are capable of specifically binding to selected targets and modulating the targets activity, e.g., through binding, aptamers may block their target's ability to function. A typical aptamer is 10-15 kDa in size (30-45 nucleotides), binds its target with sub-nanomolar affinity, and discriminates against closely related targets (e.g., aptamers will typically not bind other proteins from the same gene family). Structural studies have shown that aptamers are capable of using the same types of binding interactions (e.g., hydrogen bonding, electrostatic complementarity, hydrophobic contacts, steric exclusion) that drives affinity and specificity in antibody-antigen complexes.

Aptamers have a number of desirable characteristics for use in research and as therapeutics and diagnostics including high specificity and affinity, biological efficacy, and excellent pharmacokinetic properties. In addition, they offer specific competitive advantages over antibodies and other protein biologics. Aptamers are chemically synthesized and are readily scaled as needed to meet production demand for research, diagnostic or therapeutic applications. Aptamers are chemically robust. They are intrinsically adapted to regain activity following exposure to factors such as heat and denaturants and can be stored for extended periods (>1 yr) at room temperature as lyophilized powders. Not being bound by a theory, aptamers bound to a solid support or beads may be stored for extended periods.

Oligonucleotides in their phosphodiester form may be quickly degraded by intracellular and extracellular enzymes such as endonucleases and exonucleases. Aptamers can include modified nucleotides conferring improved characteristics on the ligand, such as improved in vivo stability or improved delivery characteristics. Examples of such modifications include chemical substitutions at the ribose and/or phosphate and/or base positions. SELEX identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′NH), 2′-fluoro (2′-F), and/or 2′-0-methyl (2′-OMe) substituents. Modifications of aptamers may also include, modifications at exocyclic amines, substitution of 4-thiouridine, substitution of 5-bromo or 5-iodo-uracil; backbone modifications, phosphorothioate or allyl phosphate modifications, methylations, and unusual base-pairing combinations such as the isobases isocytidine and isoguanosine. Modifications can also include 3 and 5′ modifications such as capping. As used herein, the term phosphorothioate encompasses one or more non-bridging oxygen atoms in a phosphodiester bond replaced by one or more sulfur atoms. In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described, e.g., in Sproat, et al., Nucl Acid Res. 19:733-738 (1991); Cotten, et al, Nucl. Acid Res. 19:2629-2635 (1991); and Hobbs, et al, Biochemistry 12:5138-5145 (1973). Other modifications are known to one of ordinary skill in the art. In certain embodiments, aptamers include aptamers with improved off-rates as described in international Patent Publication No. WO 2009012418, “Method for generating aptamers with improved off-rates,” incorporated herein by reference in its entirety. In certain embodiments aptamers are chosen from a library of aptamers. Such libraries include, but are not limited to, those described in Rohloff et al., “Nucleic Acid Ligands With Protein-like Side Chains: Modified Aptamers and Their Use as Diagnostic and Therapeutic Agents,” Molecular Therapy Nucleic Acids (2014) 3, e201. Aptamers are also commercially available (see, e.g., SomaLogic, Inc., Boulder, Colorado). In certain embodiments, the present invention may utilize any aptamer containing any modification as described herein.

### Small Molecules

In certain embodiments, the one or more agents is a small molecule. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking an enzyme active site or activating a receptor by binding to a ligand binding site).

One type of small molecule applicable to the present invention is a degrader molecule (see, e.g., Ding, et al., Emerging New Concepts of Degrader Technologies, Trends Pharmacol Sci. 2020 July; 41(7):464-474). The terms “degrader” and “degrader molecule” refer to all compounds capable of specifically targeting a protein for degradation (e.g., ATTEC, AUTAC, LYTAC, or PROTAC, reviewed in Ding, et al. 2020). Proteolysis Targeting Chimera (PROTAC) technology is a rapidly emerging alternative therapeutic strategy with the potential to address many of the challenges currently faced in modern drug development programs. PROTAC technology employs small molecules that recruit target proteins for ubiquitination and removal by the proteasome (see, e.g., Zhou et al., Discovery of a Small-Molecule Degrader of Bromodomain and Extra-Terminal (BET) Proteins with Picomolar Cellular Potencies and Capable of Achieving Tumor Regression. J. Med. Chem. 2018, 61, 462-481; Bondeson and Crews, Targeted Protein Degradation by Small Molecules, Annu Rev Pharmacol Toxicol. 2017 Jan. 6; 57: 107-123; and Lai et al., Modular PROTAC Design for the Degradation of Oncogenic BCR-ABL Angew Chem Int Ed Engl. 2016 Jan. 11; 55(2): 807-810). In certain embodiments, LYTACs are particularly advantageous for cell surface proteins as described herein (e.g., CD160).

### Genetic Modifying Agents

In certain embodiments, the one or more modulating agents may be a genetic modifying agent. The genetic modifying agents may manipulate nucleic acids (e.g., genomic DNA or mRNA). The genetic modulating agent can be used to up- or downregulate expression of a gene either by targeting a nuclease or functional domain to a DNA or RNA sequence. The genetic modifying agent may comprise an RNA-guided nuclease system (e.g., CRISPR system or IscB system), RNAi system, a zinc finger nuclease, a TALE, or a meganuclease.

### CRISPR-Cas Modification

In some embodiments, a polynucleotide of the present invention described elsewhere herein can be modified using a CRISPR-Cas and/or Cas-based system (e.g., genomic DNA or mRNA, preferably, for a disease gene). The nucleotide sequence may be or encode one or more components of a CRISPR-Cas system. For example, the nucleotide sequences may be or encode guide RNAs. The nucleotide sequences may also encode CRISPR proteins, variants thereof, or fragments thereof.

In general, a CRISPR-Cas or CRISPR system as used herein and in other documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g., CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel. 2015.10.008.

CRISPR-Cas systems can generally fall into two classes based on their architectures of their effector molecules, which are each further subdivided by type and subtype. The two classes are Class 1 and Class 2. Class 1 CRISPR-Cas systems have effector modules composed of multiple Cas proteins, some of which form crRNA-binding complexes, while Class 2 CRISPR-Cas systems include a single, multi-domain crRNA-binding protein.

In some embodiments, the CRISPR-Cas system that can be used to modify a polynucleotide of the present invention described herein can be a Class 1 CRISPR-Cas system. In some embodiments, the CRISPR-Cas system that can be used to modify a polynucleotide of the present invention described herein can be a Class 2 CRISPR-Cas system.

### Class 1 CRISPR-Cas Systems

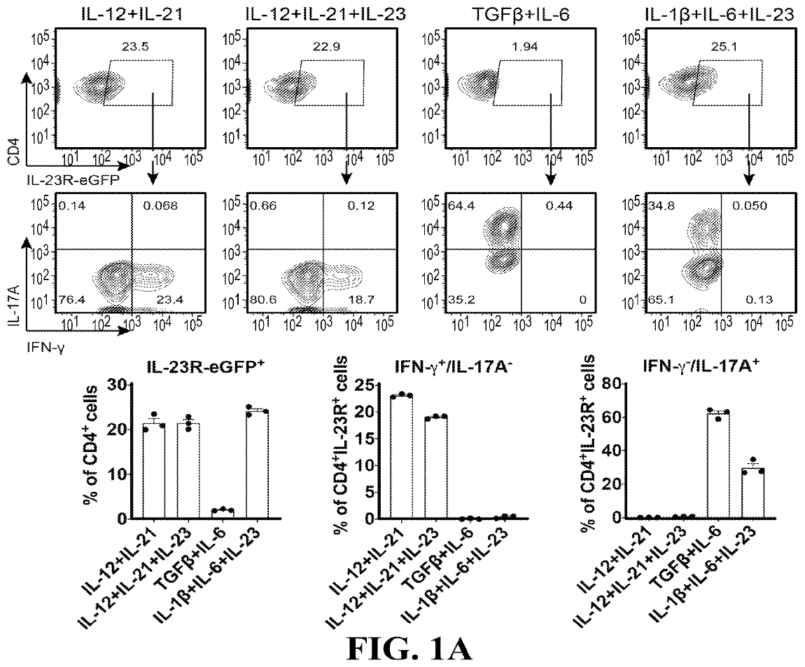

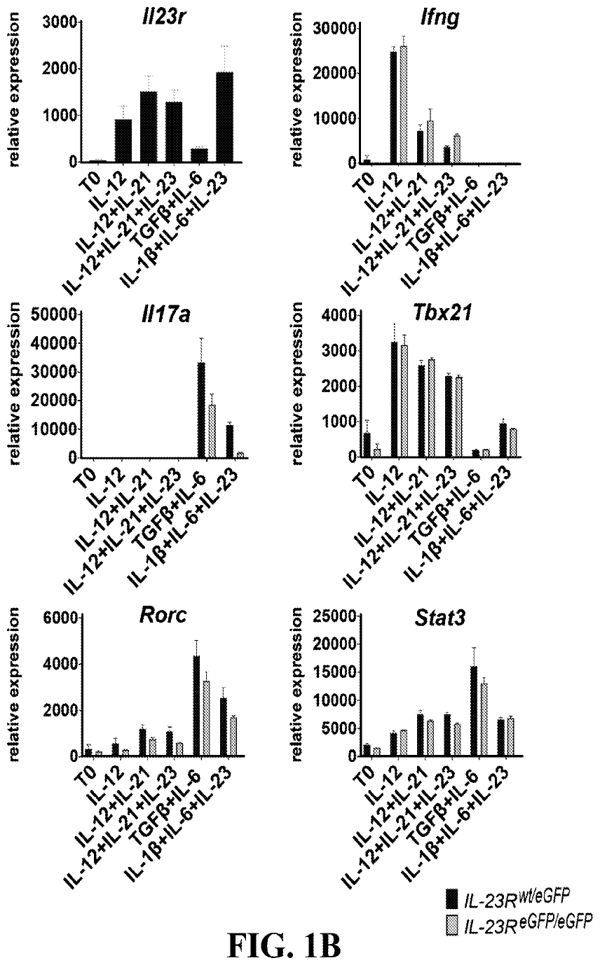

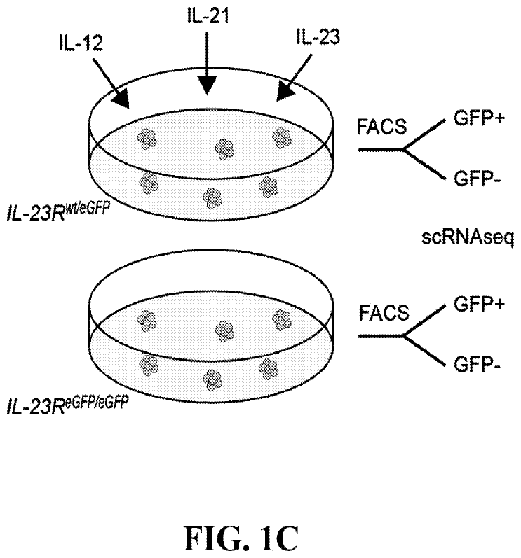

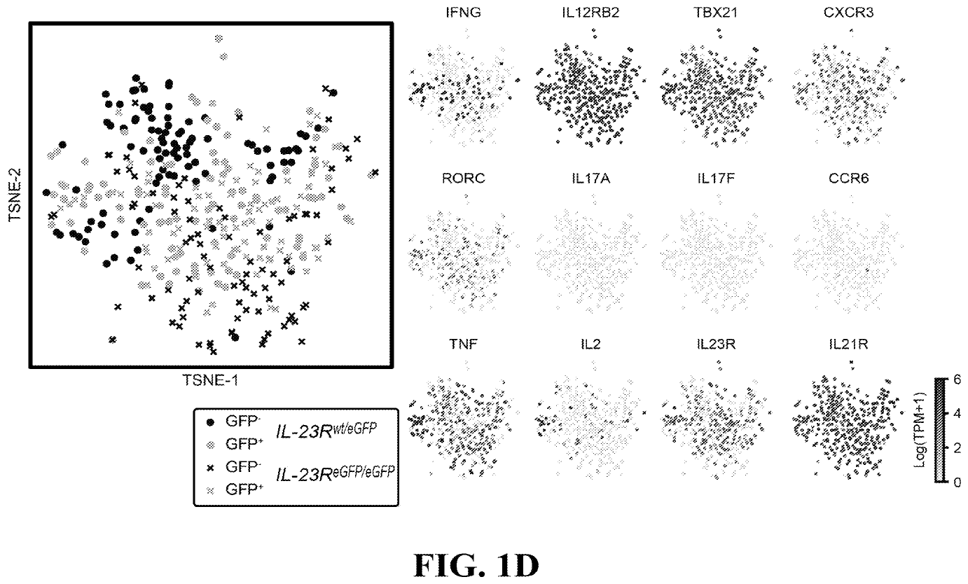

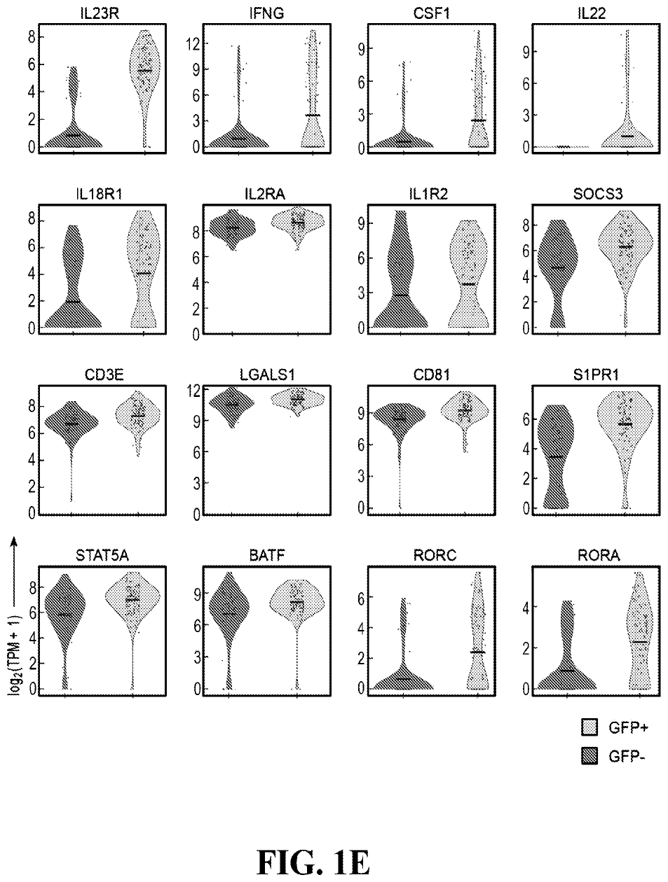

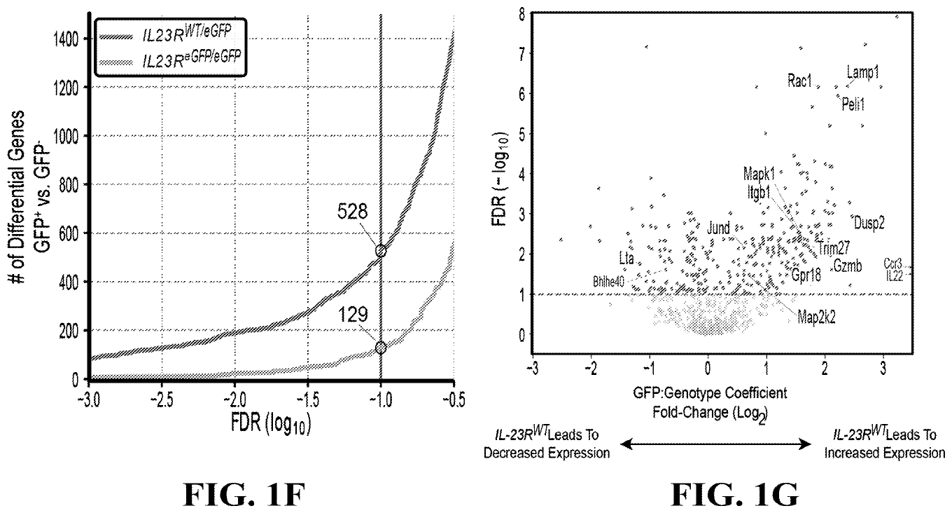

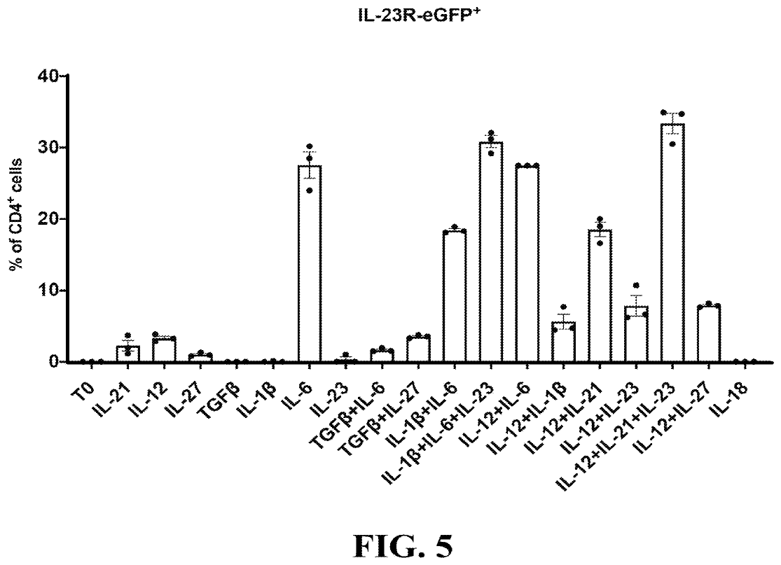

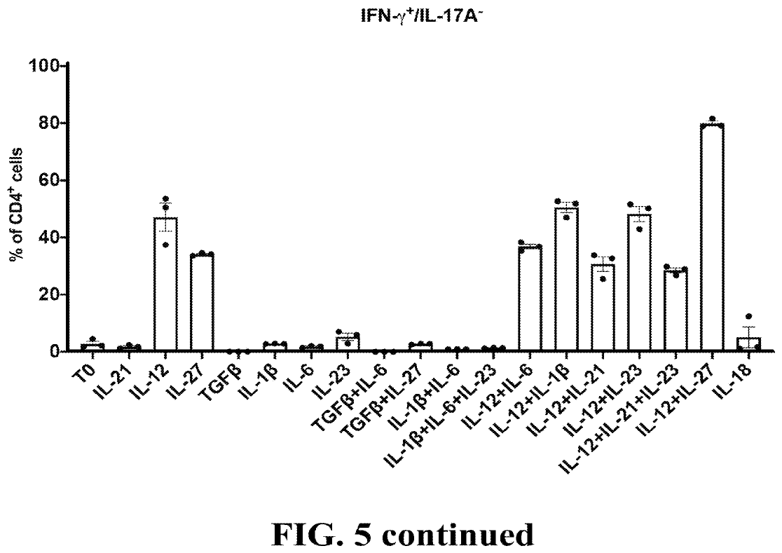

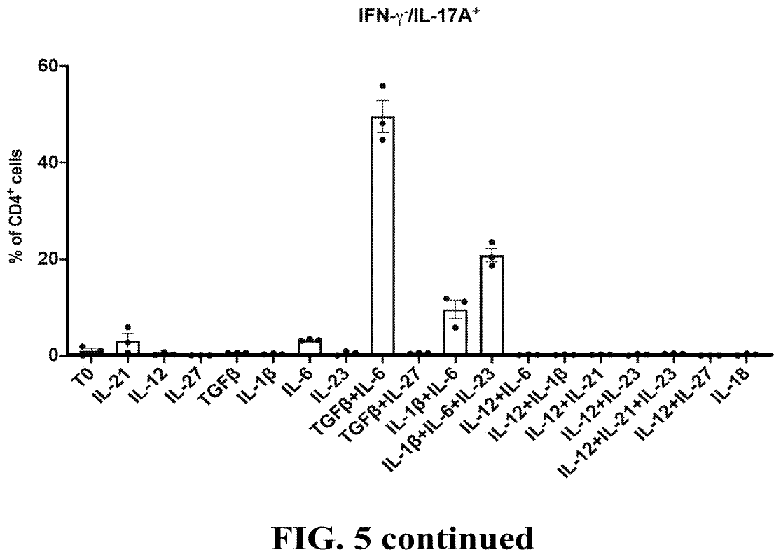

In some embodiments, the CRISPR-Cas system that can be used to modify a polynucleotide of the present invention described herein can be a Class 1 CRISPR-Cas system. Class 1 CRISPR-Cas systems are divided into Types I, II, and IV. Makarova et al. 2020. Nat. Rev. 18: 67-83, particularly as described in FIG. 1. Type I CRISPR-Cas systems are divided into 9 subtypes (I-A, I-B, I-C, I-D, I-E, I-F1, I-F2, I-F3, and IG). Makarova et al., 2020. Class 1, Type I CRISPR-Cas systems can contain a Cas3 protein that can have helicase activity. Type III CRISPR-Cas systems are divided into 6 subtypes (III-A, III-B, III-C, III-D, III-E, and III-F). Type III CRISPR-Cas systems can contain a Cas10 that can include an RNA recognition motif called Palm and a cyclase domain that can cleave polynucleotides. Makarova et al., 2020. Type IV CRISPR-Cas systems are divided into 3 subtypes. (IV-A, IV-B, and IV-C). Makarova et al., 2020. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al. 2018. The CRISPR Journal, v. 1, n5, FIG. 5.

The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas 4, DNA nuclease), CRISPR associated Rossman fold (CARF) domain containing proteins, and/or RNA transcriptase.

The backbone of the Class 1 CRISPR-Cas system effector complexes can be formed by RNA recognition motif domain-containing protein(s) of the repeat-associated mysterious proteins (RAMPs) family subunits (e.g., Cas 5, Cas6, and/or Cas7). RAMP proteins are characterized by having one or more RNA recognition motif domains. In some embodiments, multiple copies of RAMPs can be present. In some embodiments, the Class I CRISPR-Cas system can include 1, 2, 3, 4, 5, 6, 7, 8, 9, 10, 11, 12 or more Cas5, Cas6, and/or Cas 7 proteins. In some embodiments, the Cas6 protein is an RNAse, which can be responsible for pre-crRNA processing. When present in a Class 1 CRISPR-Cas system, Cas6 can be optionally physically associated with the effector complex.

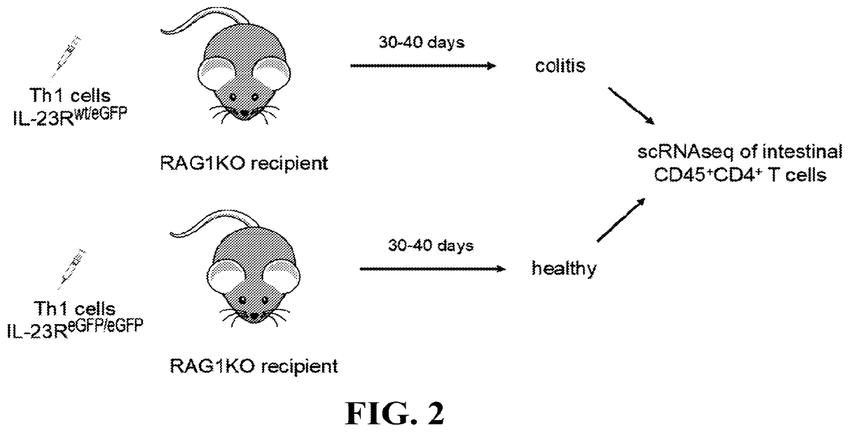

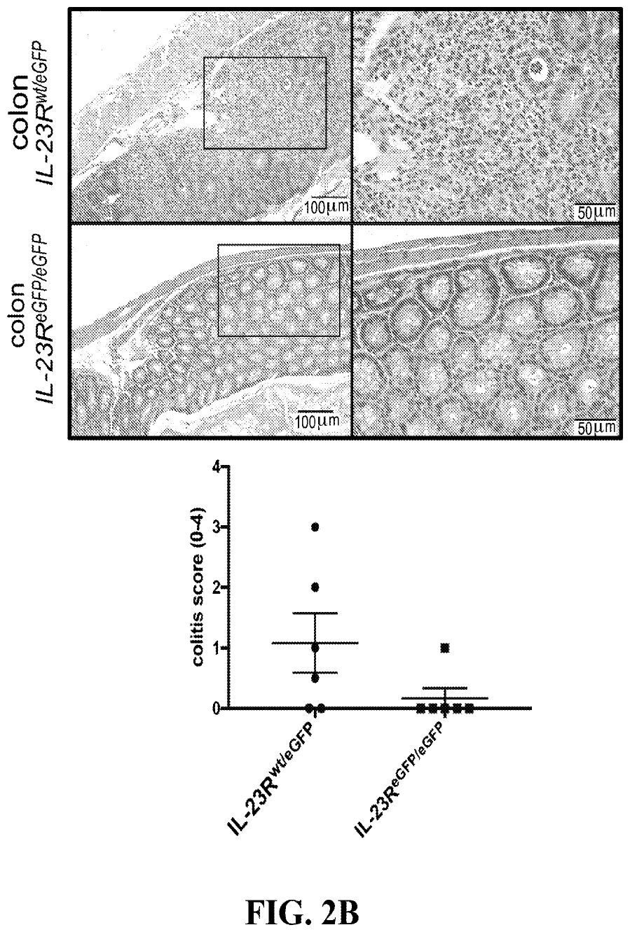

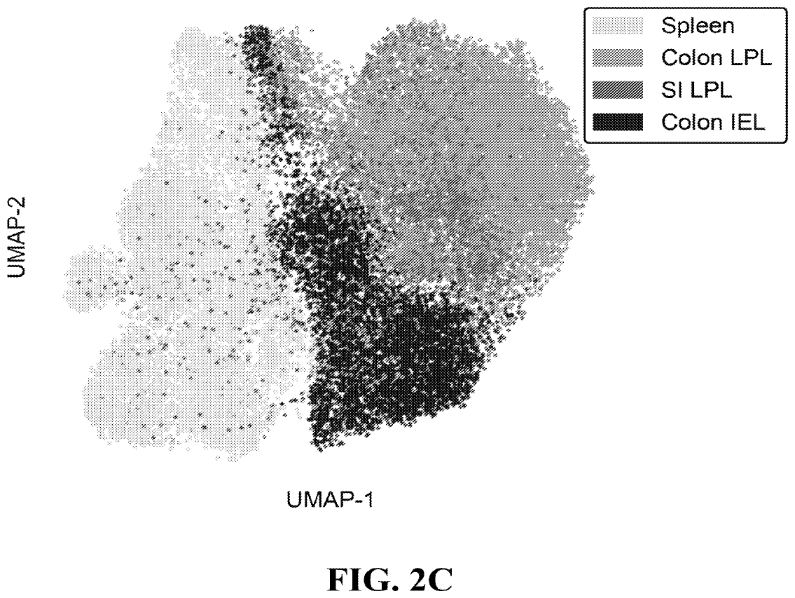

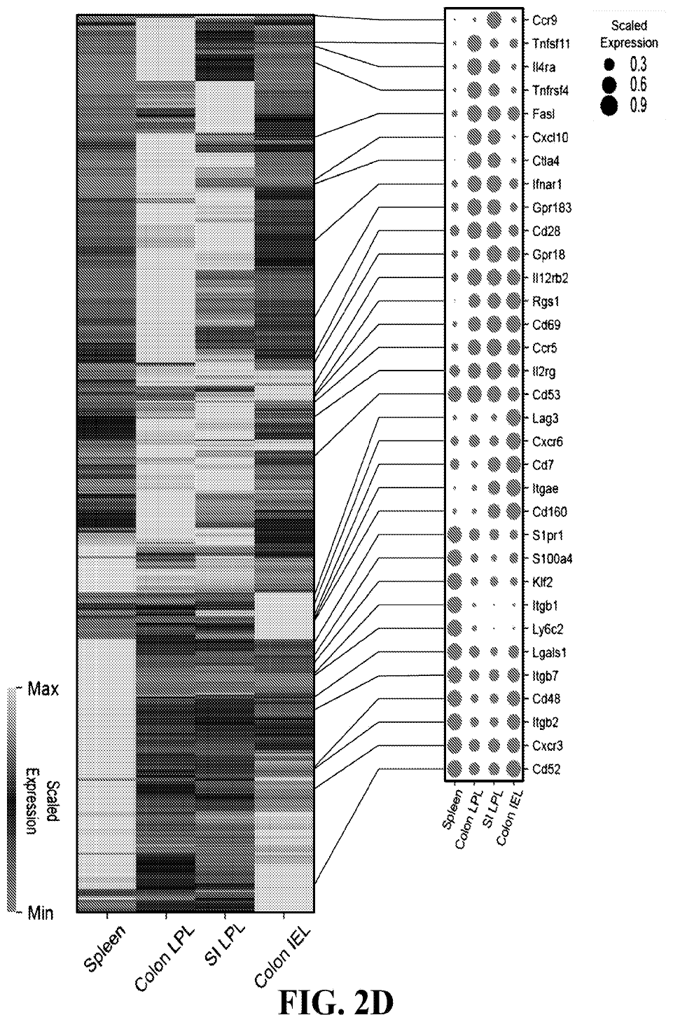

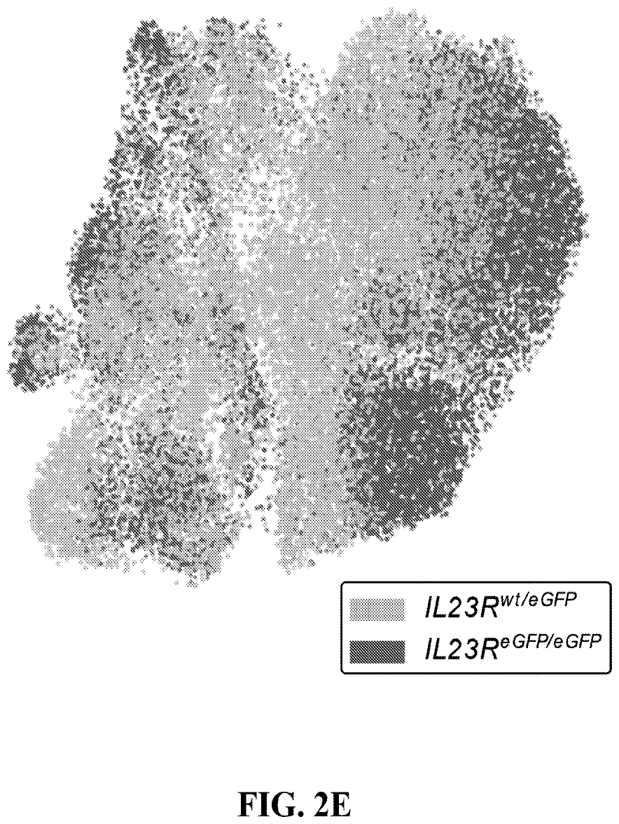

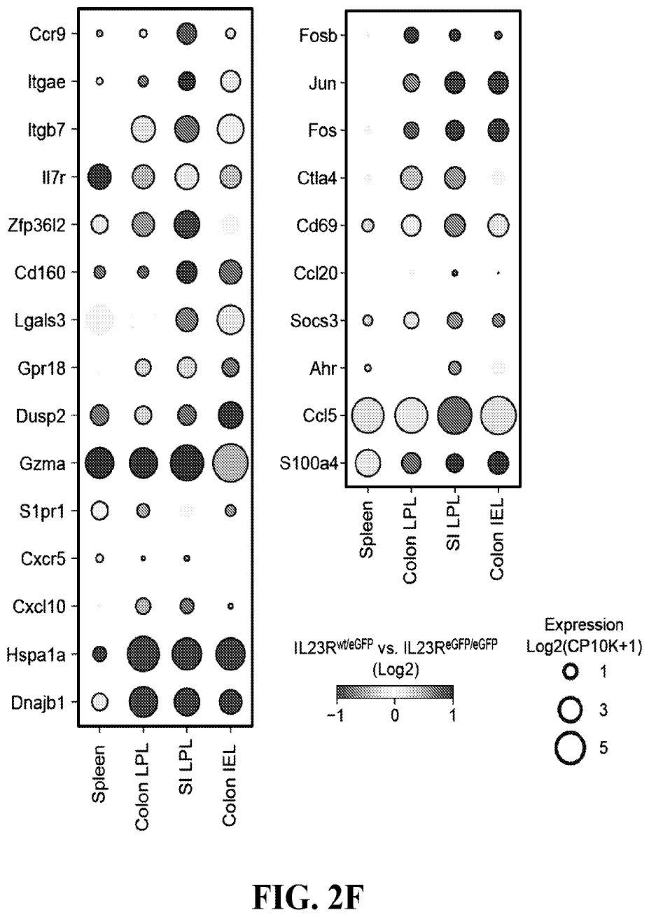

Class 1 CRISPR-Cas system effector complexes can, in some embodiments, also include a large subunit. The large subunit can be composed of or include a Cas8 and/or Cas10 protein. See, e.g., FIGS. 1 and 2. Koonin E V, Makarova K S. 2019. Phil. Trans. R. Soc. B 374: 20180087, DOI: 10.1098/rstb.2018.0087 and Makarova et al. 2020.

Class 1 CRISPR-Cas system effector complexes can, in some embodiments, include a small subunit (for example, Cas11). See, e.g., FIGS. 1 and 2. Koonin E V, Makarova K S. 2019 Origins and Evolution of CRISPR-Cas systems. Phil. Trans. R. Soc. B 374: 20180087, DOI: 10.1098/rstb.2018.0087.

In some embodiments, the Class 1 CRISPR-Cas system can be a Type I CRISPR-Cas system. In some embodiments, the Type I CRISPR-Cas system can be a subtype I-A CRISPR-Cas system. In some embodiments, the Type I CRISPR-Cas system can be a subtype I-B CRISPR-Cas system. In some embodiments, the Type I CRISPR-Cas system can be a subtype I-C CRISPR-Cas system. In some embodiments, the Type I CRISPR-Cas system can be a subtype I-D CRISPR-Cas system. In some embodiments, the Type I CRISPR-Cas system can be a subtype I-E CRISPR-Cas system. In some embodiments, the Type I CRISPR-Cas system can be a subtype I-F1 CRISPR-Cas system. In some embodiments, the Type I CRISPR-Cas system can be a subtype I-F2 CRISPR-Cas system. In some embodiments, the Type I CRISPR-Cas system can be a subtype I-F3 CRISPR-Cas system. In some embodiments, the Type I CRISPR-Cas system can be a subtype I-G CRISPR-Cas system. In some embodiments, the Type I CRISPR-Cas system can be a CRISPR Cas variant, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems as previously described.

In some embodiments, the Class 1 CRISPR-Cas system can be a Type III CRISPR-Cas system. In some embodiments, the Type III CRISPR-Cas system can be a subtype III-A CRISPR-Cas system. In some embodiments, the Type III CRISPR-Cas system can be a subtype III-B CRISPR-Cas system. In some embodiments, the Type III CRISPR-Cas system can be a subtype III-C CRISPR-Cas system. In some embodiments, the Type III CRISPR-Cas system can be a subtype III-D CRISPR-Cas system. In some embodiments, the Type III CRISPR-Cas system can be a subtype III-E CRISPR-Cas system. In some embodiments, the Type III CRISPR-Cas system can be a subtype III-F CRISPR-Cas system.

In some embodiments, the Class 1 CRISPR-Cas system can be a Type IV CRISPR-Cas-system. In some embodiments, the Type IV CRISPR-Cas system can be a subtype IV-A CRISPR-Cas system. In some embodiments, the Type IV CRISPR-Cas system can be a subtype IV-B CRISPR-Cas system. In some embodiments, the Type IV CRISPR-Cas system can be a subtype IV-C CRISPR-Cas system.

The effector complex of a Class 1 CRISPR-Cas system can, in some embodiments, include a Cas3 protein that is optionally fused to a Cas2 protein, a Cas4, a Cas5, a Cas6, a Cas7, a Cas8, a Cas10, a Cas11, or a combination thereof. In some embodiments, the effector complex of a Class 1 CRISPR-Cas system can have multiple copies, such as 1, 2, 3, 4, 5, 6, 7, 8, 9, 10, 11, 12, 13, or 14, of any one or more Cas proteins.

### Class 2 CRISPR-Cas Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), CasX, and/or Cas14.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (WO 2019/005884, WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423.

### Split CRISPR-Cas Systems

In some embodiments, the CRISPR-Cas system is a split CRISPR-Cas system. See e.g., Zetche et al., 2015. Nat. Biotechnol. 33(2): 139-142 and WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein are attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C·G base pair into a T·A base pair (Komor et al. 2016. Nature. 533:420-424; Nishida et al. 2016. Science. 353; and Li et al. Nat. Biotech. 36:324-327) and ABEs convert an A·T base pair to a G·C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471. Base editors may be further engineered to optimize conversion of nucleotides (e.g. A:T to G:C). Richter et al. 2020. Nature Biotechnology. doi.org/10.1038/s41587-020-0453-z.

Other Example Type V base editing systems are described in WO 2018/213708, WO 2018/213726, PCT/US2018/067207, PCT/US2018/067225, and PCT/US2018/067307 which are incorporated by referenced herein.

In certain example embodiments, the base editing system may be a RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA based editor may be used to delete or introduce a post-translation modification site in the expressed mRNA. In contrast to DNA base editors, whose edits are permanent in the modified cell, RNA base editors can provide edits where finer temporal control may be needed, for example in modulating a particular immune response. Example Type VI RNA-base editing systems are described in Cox et al. 2017. Science 358: 1019-1027, WO 2019/005884, WO 2019/005886, WO 2019/071048, PCT/US20018/05179, PCT/US2018/067207, which are incorporated herein by reference. An example FnCas9 system that may be adapted for RNA base editing purposes is described in WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

### Prime Editors

In some embodiments, a polynucleotide of the present invention described elsewhere herein can be modified using a prime editing system (See e.g., Anzalone et al. 2019. Nature. 576: 149-157). Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion, and combinations thereof. Generally, a prime editing system, as exemplified by PE1, PE2, and PE3 (Id.), can include a reverse transcriptase fused or otherwise coupled or associated with an RNA-programmable nickase, and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g., a prime editing guide molecule or peg guide molecule) directly into the target site in the target polynucleotide. See e.g., Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g. is a Cas9 nickase). In some embodiments, the Cas polypeptide is fused to the reverse transcriptase. In some embodiments, the Cas polypeptide is linked to the reverse transcriptase.

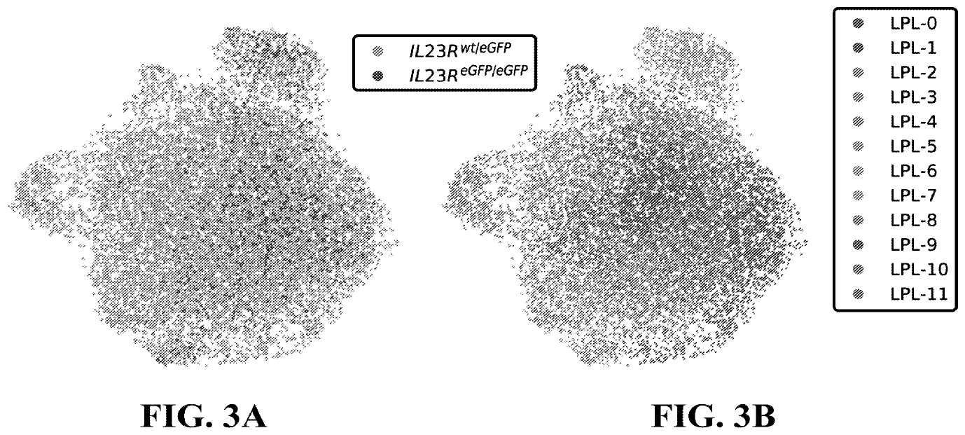

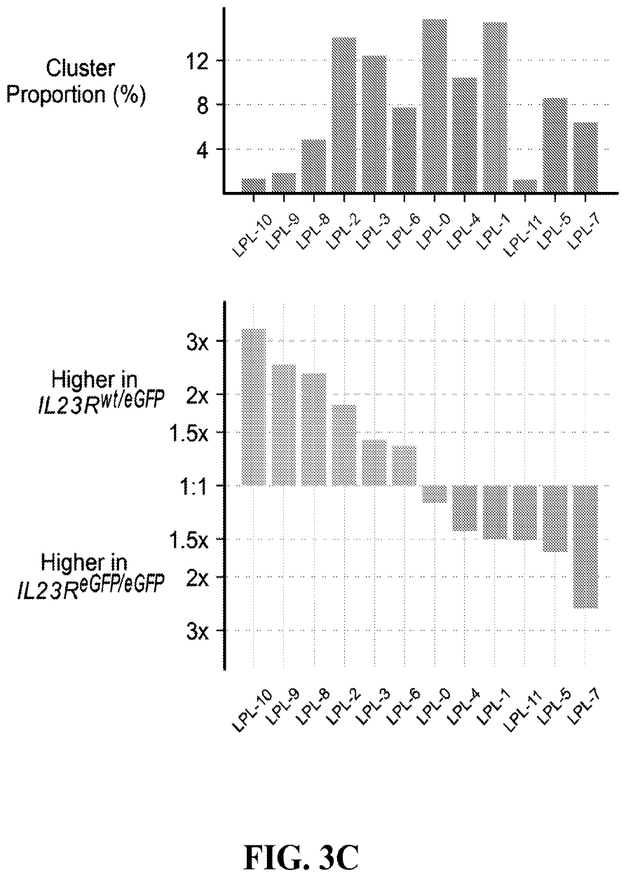

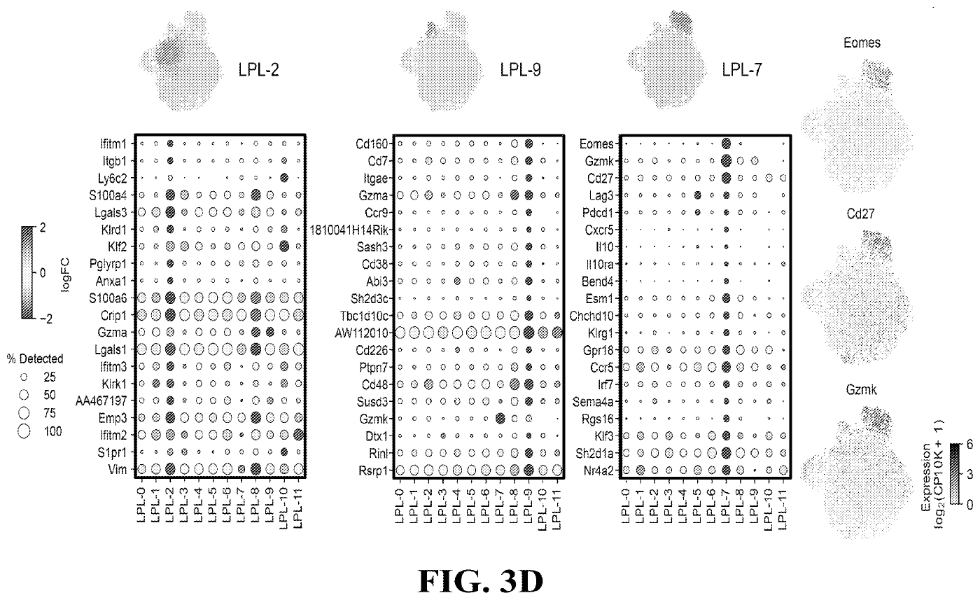

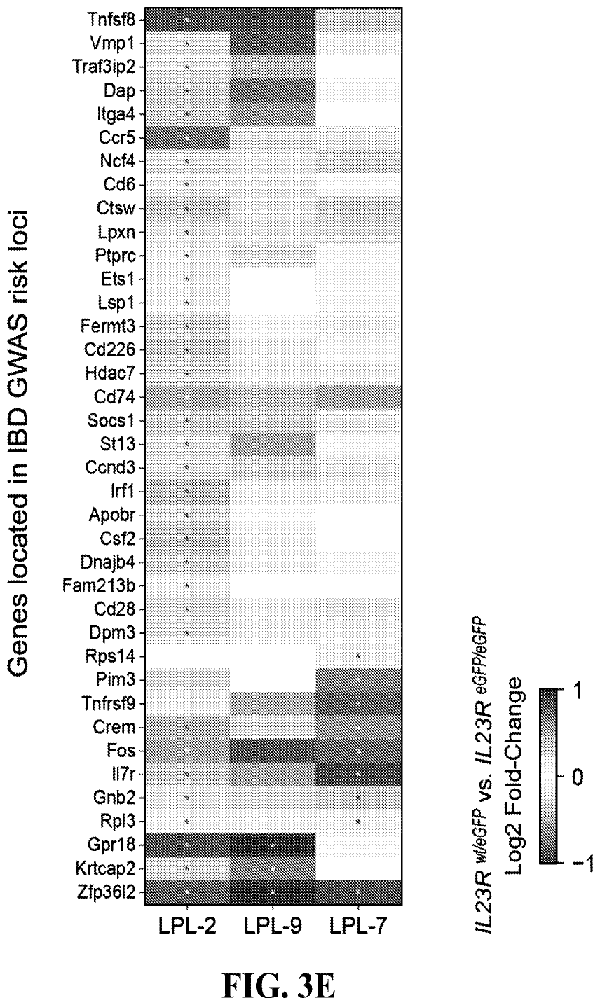

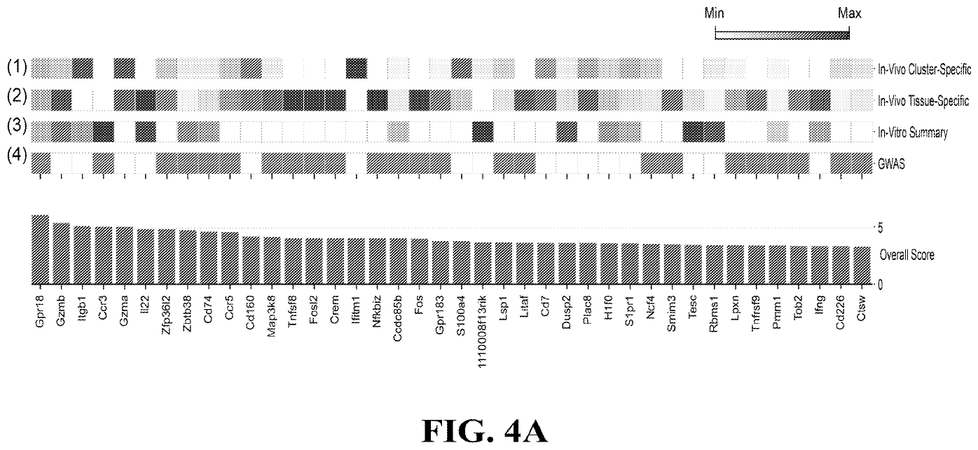

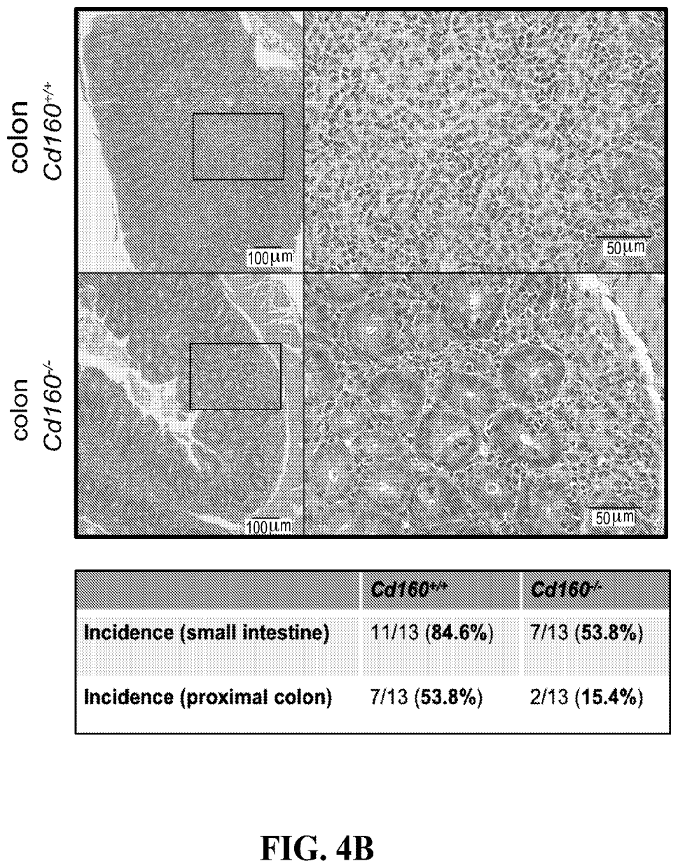

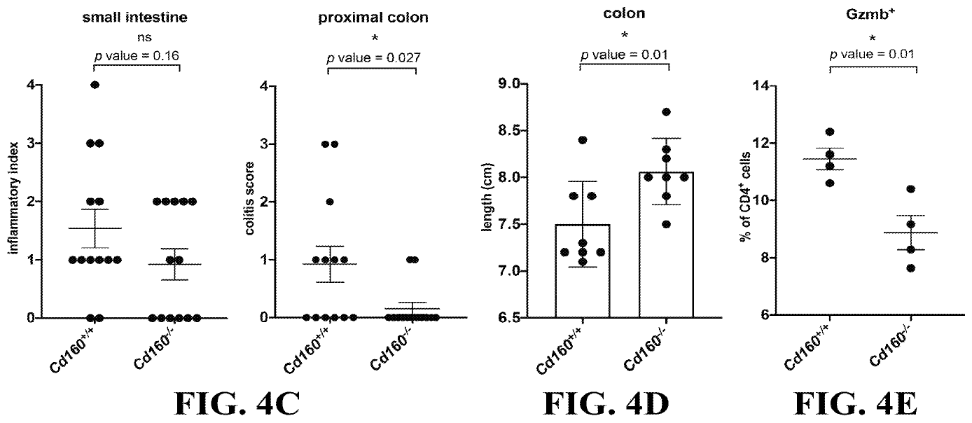

In some embodiments, the prime editing system can be a PE1 system or variant thereof, a PE2 system or variant thereof, or a PE3 (e.g., PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposase (CAST) Systems

In some embodiments, a polynucleotide of the present invention described elsewhere herein can be modified using a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

### Guide Molecules

The CRISPR-Cas or Cas-Based system described herein can, in some embodiments, include one or more guide molecules. The terms guide molecule, guide sequence and guide polynucleotide, refer to polynucleotides capable of guiding Cas to a target genomic locus and are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in PCT US2019/045582, specifically paragraphs [0178]-[0333]. which is incorporated herein by reference.

### Target Sequences, PAMs, and PFSs

### Target Sequences

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table A below shows several Cas polypeptides and the PAM sequence they recognize.

TABLE A
Example PAM Sequences

| | |
|---|---|
| Cas Protein | PAM Sequence |
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including | TTTV |
| LbCpf1 and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |

In a preferred embodiment, the CRISPR effector protein may recognize a 3′ PAM. In certain embodiments, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead, such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAsl3a) have a specific discrimination against G at the 3′end of the target RNA. The presence of a C at the corresponding crRNA repeat site can indicate that nucleotide pairing at this position is rejected. However, some Cas13 proteins (e.g., LwaCAsl3a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### IscBs

In some embodiments, the nucleic acid-guided nucleases herein may be an IscB protein. An IscB protein may comprise an X domain and a Y domain as described herein. In some examples, the IscB proteins may form a complex with one or more guide molecules. In some cases, the IscB proteins may form a complex with one or more hRNA molecules which serve as a scaffold molecule and comprise guide sequences. In some examples, the IscB proteins are CRISPR-associated proteins, e.g., the loci of the nucleases are associated with an CRISPR array. In some examples, the IscB proteins are not CRISPR-associated.

In some examples, the IscB protein may be homolog or ortholog of IscB proteins described in Kapitonov V V et al., ISC, a Novel Group of Bacterial and Archaeal DNA Transposons That Encode Cas9 Homologs, J Bacteriol. 2015 Dec. 28; 198(5):797-807. doi: 10.1128/JB.00783-15, which is incorporated by reference herein in its entirety.

In some embodiments, the IscBs may comprise one or more domains, e.g., one or more of a X domain (e.g., at N-terminus), a RuvC domain, a Bridge Helix domain, and a Y domain (e.g., at C-terminus). In some examples, the nucleic-acid guided nuclease comprises an N-terminal X domain, a RuvC domain (e.g., including a RuvC-I, RuvC-II, and RuvC-III subdomains), a Bridge Helix domain, and a C-terminal Y domain. In some examples, the nucleic-acid guided nuclease comprises In some examples, the nucleic-acid guided nuclease comprises an N-terminal X domain, a RuvC domain (e.g., including a RuvC-I, RuvC-II, and RuvC-III subdomains), a Bridge Helix domain, an HNH domain, and a C-terminal Y domain.

In some embodiments, the nucleic acid-guided nucleases may have a small size. For example, the nucleic acid-guided nucleases may be no more than 50, no more than 100, no more than 150, no more than 200, no more than 250, no more than 300, no more than 350, no more than 400, no more than 450, no more than 500, no more than 550, no more than 600, no more than 650, no more than 700, no more than 750, no more than 800, no more than 850, no more than 900, no more than 950, or no more than 1000 amino acids in length.

In some examples, the IscB protein shares at least 80%, at least 85%, at least 90%, at least 95%, at least 99%, or 100% sequence identity with a IscB protein selected from Table 1.

TABLE 1

| No. | Proteins | Sequences | |
|---|---|---|---|
| 1 | IscB(−HNH) | 1 | mstdatlirt tpshaeadat dtlvatplmp prrvispwpg pgegqslmri pvvdirgmal |
| | EFH81386 | 61 | mpctpakarh llksgnarpk rnklglfyvq lsyeqepdnq slvagvdpgs kfeglsvvgt |
| | | 121 | kdtvlnlmve apdhvkgavq trrtmrrarr qrkwrrpkrf hnrlnrmqri ppstrsrwea |
| | | 181 | karivahlrt ilpftdvvve dvqavtrkgk ggtwngsfsp vqvgkehlyr llramgltlh |
| | | 241 | lregwqtkel reqhglkktk skskqsfesh avdswvlaas isgaehptct rlwymvpail |
| | | 301 | hrrqlhrlqa skggvrkpyg gtrslgvkrg tlvehkkygr ctvggvdrkr ntislheyrt |
| | | 361 | ntrltqaakv etcrvltwls wrswllrgkr tsskgkgshs s (SEQ ID NO: 1) |
| 2 | IscB(+HNH) | 1 | mqpakqqnwv fqingdkqpl dminpgrcre lqnrgklasf rrfpyvviqq qtienpqtke |
| | TAE54104.1 | 61 | yilkidpgsq wtgfaiqcgn dilfraelnh rgeaikfdlv krawfrrgrr srnlryrkkr |
| | | 121 | lnrakpegwl apsirhrvlt vetwikrfmr ycpiawieie qvrfdtqkla npeidgveyq |
| | | 181 | qgelqgyevr eyllqkwgrk caycgtenvp levehiqsks kggssrignl tlachvcnvk |
| | | 241 | kgnldvrdfl akspdilngv lenstkplkd aaavnstrya ivkmaksice nvkcssgart |
| | | 301 | kmnrvrqgle kthsldaacv gesgasirvl tdrpllitck ghgsrqsirv nasgfpavkn |
| | | 361 | aktvfthiaa gdvvrftigk drkkaqagty tarvktptpk gfevlidgar islstmsnvv |
| | | 421 | fvhrsdgygy el (SEQ ID NO: 2) |
| 3 | IscB(+HNH) | 1 | mavfvidkhk rplmpcsekr arlllergra vvhrqvpfvi rlkdrtvqhs avqplrvald |
| | WP_038093640.1 | 61 | pgsratgmal vrekntvdtg tgevyreria lnlfelvhrg hrireqldqr rnfrrrrrga |
| | | 121 | nlryraprfd nrrrppgwla pslqhrvdtt mawvrrlerw apasaigiet vrfdtqrlqn |
| | | 181 | peisgveyqq galagcevre yllekwgrkc aycgaenvpl eiehivpksr ggsdrvsnla |
| | | 241 | lacracnqak gnrdvrafla dqperlaril aqakaplkda aavnatrwal yralvdtglp |
| | | 301 | veagtggrtk wnrtrlglpk thaldalcvg qvdqvrhwrv pvlgircagr gsyrrtrltr |
| | | 361 | hgfprgyltr nksafgfqtg dliravvtkg kkagtylgri airasgsfni qtpmgvvqgi |
| | | 421 | hhrfctllqr adgygyfvqp kpteaalssp rlkagvssag n (SEQ ID NO: 3) |
| 4 | IscB(+HNH) | 1 | mttnvvfvid tnqkplqpcs aavarklllr gkaamfrryp aviilkkevd svgkpkielr |
| | WP_052490348.1 | 61 | idpgskytgf alvdskdnad fiiwgteleh rgaaickelt krsairrsrr nrktryrkkr |
| | | 121 | ferrkpegwl apslqhrvdt tltwvkrick fvpimsisve qvkfdlqkle nsdiqgieyq |
| | | 181 | qgtlagytlr eallehwgrk caycdvenvf leiehiypks kggsdkfsnl tlachkcnin |
| | | 241 | kgnksidefl lsdhkrleqi klhqkktlkd aaavnatrkk lvttlqektf lnvlvsdgas |
| | | 301 | tkmtrlsssl akrhwidagc vnttlivilk tlqplqvkcn ghgnkqfvtm daygfprksy |
| | | 361 | epkkvrkdwk agdiirvtkk dgtmlmgrvk kaakklvyip fggkeasfss enakaihrsd |
| | | 421 | gyrysfaaid sellqkmat (SEQ ID NO: 4) |
| 5 | IscB(+HNH) | 1 | mpnkyafvld skgklldptk skkawylirk gkaslveeyp liiklkrevp kdqvnsdkli |
| | WP_015325818.1 | 61 | lgiddgtkkv gfalvqkcqt knkvlfkavm eqrqdvskkm eerrgyrryr rshkryrpar |
| | | 121 | fdnrssskrk grippsilqk kqailrvvnk lkkyiridki vledvsidir kltegrelyn |
| | | 181 | weyqesnrld enlrkatlyr ddctcqlegt tetmlhahhi mprrdggads iynlitlcka |
| | | 241 | chkdkvdnne yqykdqflai idskelsdlk sashvmqgkt wlrdklskia qleitsggnt |
| | | 301 | ankridyeie kshsndaict tgllpvdnid dikeyyikpl rkkskakike lkcfrqrdlv |
| | | 361 | kytkrngety tgyitslrik nnkynskven fstlkgkifr gygfrnltll nrpkglmiv |
| | | | (SEQ ID NO: 5) |
| 6 | sp\|G3ECR1\|CAS9_ | 1 | mlfnkciiis inldfsnkek cmtkpysigl digtnsvgwa vitdnykvps kkmkvlgnts |
| | STRTR | 61 | kkyikknllg vllfdsgita egrrlkrtar rrytrrrnri lylqeifste matlddaffq |
| | | 121 | rlddsflvpd dkrdskypif gnlveekvyh defptiyhlr kyladstkka dlrlvylala |
| | | 181 | hmikyrghfl iegefnsknn diqknfqdfl dtynaifesd lslenskqle eivkdkiskl |
| | | 241 | ekkdrilklf pgeknsgifs eflklivgnq adfrkcfnld ekaslhfske sydedletll |
| | | 301 | gyigddysdv flkakklyda illsgfltvt dneteaplss amikrynehk edlallkeyi |
| | | 361 | rnislktyne vfkddtkngy agyidgktnq edfyvylknl laefegadyf lekidredfl |
| | | 421 | rkqrtfdngs ipyqihlqem raildkqakf ypflaknker iekiltfrip yyvgplargn |
| | | 481 | sdfawsirkr nekitpwnfe dvidkessae afinrmtsfd lylpeekvlp khsllyetfn |
| | | 541 | vyneltkvrf iaesmrdyqf ldskqkkdiv rlyfkdkrkv tdkdiieylh aiygydgiel |
| | | 601 | kgiekqfnss lstyhdllni indkefldds sneaiieeii htltifedre mikqrlskfe |
| | | 661 | nifdksvlkk lsrrhytgwg klsaklingi rdeksgntil dyliddgisn rnfmqlihdd |
| | | 721 | alsfkkkiqk aqiigdedkg nikevvkslp gspaikkgil qsikivdelv kvmggrkpes |
| | | 781 | ivvemarenq ytnqgksnsq qrlkrleksl kelgskilke nipaklskid nnalqndrly |
| | | 841 | lyylqngkdm ytgddldidr lsnydidhii pqaflkdnsi dnkvlvssas nrgksddfps |
| | | 901 | levvkkrktf wyqllkskli sqrkfdnltk aerggllped kagfiqrqlv etrqitkhva |
| | | 961 | rlldekfnnk kdennravrt vkiitlkstl vsqfrkdfel ykvreindfh hahdaylnav |
| | | 1021 | iasallkkyp klepefvygd ypkynsfrer ksatekvyfy snimnifkks isladgrvie |
| | | 1081 | rplievneet gesvwnkesd latvrrvlsy pqvnvvkkve eqnhgldrgk pkglfnanls |
| | | 1141 | skpkpnsnen lvgakeyldp kkyggyagis nsfavlvkgt iekgakkkit nvlefqgisi |
| | | 1201 | ldrinyrkdk lnfllekgyk dieliielpk yslfelsdgs rrmlasilst nnkrgeihkg |
| | | 1261 | nqiflsqkfv kllyhakris ntinenhrky venhkkefee lfyyilefne nyvgakkngk |
| | | 1321 | llnsafqswq nhsidelcss figptgserk glfeltsrgs aadfeflgvk ipryrdytps |
| | | 1381 | slikdatlih qsvtglyetr idlaklgeg (SEQ ID NO: 6) |
| 7 | sp\|J7RUA5\|CAS9_ | 1 | mkrnyilgld igitsvgygi idyetrdvid agvrlfkean vennegrrsk rgarrlkrrr |
| | STAAU | 61 | rhriqrvkkl lfdynlltdh selsginpye arvkglsqkl seeefsaall hlakrrgvhn |
| | | 121 | vneveedtgn elstkeqisr nskaleekyv aelqlerlkk dgevrgsinr fktsdyvkea |
| | | 181 | kqllkvqkay hqldqsfidt yidlletrrt yyegpgegsp fgwkdikewy emlmghctyf |
| | | 241 | peelrsvkya ynadlynaln dlnnlvitrd enekleyyek fqiienvfkq kkkptlkqia |
| | | 301 | keilvneedi kgyrvtstgk peftnlkvyh dikditarke iienaelldq iakiltiyqs |
| | | 361 | sediqeeltn lnseltqeei eqisnlkgyt gthnlslkai nlildelwht ndnqiaifnr |
| | | 421 | lklvpkkvdl sqqkeipttl vddfilspvv krsfiqsikv inaiikkygl pndiiielar |
| | | 481 | eknskdagkm inemqkrnrq tnerieeiir ttgkenakyl iekiklhdmq egkclyslea |
| | | 541 | ipledllnnp fnyevdhiip rsvsfdnsfn nkvlvkqeen skkgnrtpfq ylsssdskis |
| | | 601 | yetfkkhiln lakgkgrisk tkkeylleer dinrfsvqkd finrnlvdtr yatrglmnll |
| | | 661 | rsyfrvnnld vkvksinggf tsflrrkwkf kkernkgykh haedaliian adfifkewkk |
| | | 721 | ldkakkvmen qmfeekqaes mpeieteqey keifitphqi khikdfkdyk yshrvdkkpn |
| | | 781 | relindtlys trkddkgntl ivnninglyd kdndklkkli nkspekllmy hhdpqtyqkl |
| | | 841 | klimeqygde knplykyyee tgnyltkysk kdngpvikki kyygnklnah lditddypns |
| | | 901 | rnkvvklslk pyrfdvyldn gvykfvtvkn ldvikkenyy evnskcyeea kklkkisnqa |
| | | 961 | efiasfynnd likingelyr vigvnndlln rievnmidit yreylenmnd krppriikti |
| | | 1021 | asktqsikky stdilgnlye vkskkhpgii kkg (SEQ ID NO: 7) |
| 8 | Streptococcus_ | 1 | kysigldigt nsvgwavitd eykvpskkfk vlgntdrhsi kknligallf dsgetaeatr |
| | pyogenes_SF370 | 61 | lkrtarrryt rrknricylq eifsnemakv ddsffhrlee sflveedkkh erhpifgniv |
| | | 121 | devayhekyp tiyhlrkklv dstdkadlrl iylalahmik frghfliegd lnpdnsdvdk |
| | | 181 | lfiqlvqtyn qlfeenpina sgvdakails arlsksrrle nliaqlpgek knglfgnlia |
| | | 241 | lslgltpnfk snfdlaedak lqlskdtydd dldnllaqig dqyadlflaa knlsdaills |
| | | 301 | dilrvnteit kaplsasmik rydehhqdlt llkalvrqql pekykeiffd qskngyagyi |
| | | 361 | dggasqeefy kfikpilekm dgteellvkl nredllrkqr tfdngsiphq ihlgelhail |
| | | 421 | rrqedfypfl kdnrekieki ltfripyyvg plargnsrfa wmtrkseeti tpwnfeevvd |
| | | 481 | kgasaqsfie rmtnfdknlp nekvlpkhsl lyeyftvyne ltkvkyvteg mrkpaflsge |
| | | 541 | qkkaivdllf ktnrkvtvkq lkedyfkkie cfdsveisgv edrfnaslgt yhdllkiikd |
| | | 601 | kdfldneene diledivltl tlfedremie erlktyahlf ddkvmkqlkr rrytgwgrls |
| | | 661 | rklingirdk qsgktildfl ksdgfanrnf mqlihddslt fkediqkaqv sgqgdslheh |
| | | 721 | ianlagspai kkgilqtvkv vdelvkvmgr hkpeniviem arenqttqkg qknsrermkr |
| | | 781 | ieegikelgs qilkehpven tqlqneklyl yylqngrdmy vdqeldinrl sdydvdhivp |
| | | 841 | qsflkddsid nkvltrsdkn rgksdnvpse evvkkmknyw rqllnaklit qrkfdnltka |
| | | 901 | ergglseldk agfikrqlve trqitkhvaq ildsrmntky dendklirev kvitlksklv |
| | | 961 | sdfrkdfqfy kvreinnyhh ahdaylnavv gtalikkypk lesefvygdy kvydvrkmia |
| | | 1021 | kseqeigkat akyffysnim nffkteitla ngeirkrpli etngetgeiv wdkgrdfatv |
| | | 1081 | rkvlsmpqvn ivkktevqtg gfskesilpk rnsdkliark kdwdpkkygg fdsptvaysv |
| | | 1141 | lvvakvekgk skklksvkel lgitimerss feknpidfle akgykevkkd liiklpkysl |
| | | 1201 | felengrkrm lasagelqkg nelalpskyv nflylashye klkgspedne qkqlfveqhk |
| | | 1261 | hyldeiieqi sefskrvila danldkvlsa ynkhrdkpir eqaeniihlf tltnlgapaa |
| | | 1321 | fkyfdttidr krytstkevl datlihqsit glyetridls glggd (SEQ ID NO: 8) |

| No. | Proteins | Domains and amino acid positions |
|---|---|---|
| 1 | IscB(−HNH) | X domain: 51-97 |
| | EFH81386 | RuvC-I: 104-118 |
| | | Bridge Helix: 140-160 |
| | | RuvC-II: 169-212 |
| | | RuvC-III: 226-278 |
| 2 | IscB(+HNH) | X domain: 11-56 |
| | TAE54104.1 | RuvC-I: 63-77 |
| | | Bridge Helix: 100-121 |
| | | RuvC-II: 129-172 |
| | | HNH: 211-243 |
| | | RuvC-III: 279-321 |
| 3 | IscB(+HNH) | X domain: 4-50 |
| | WP_038093640.1 | RuvC-I: 57-71 |
| | | Bridge Helix: 108-129 |
| | | RuvC-II: 138-181 |
| | | HNH: 220-252 |
| | | RuvC-III: 288-330 |
| 4 | IscB(+HNH) | X domain: 7-52 |
| | WP_052490348.1 | RuvC-I: 59-73 |
| | | Bridge Helix: 100-121 |
| | | RuvC-II: 129-172 |
| | | HNH: 211-243 |
| | | RuvC-III: 279-322 |
| 5 | IscB(+HNH) | X domain: 7-52 |
| | WP_015325818.1 | RuvC-I: 61-75 |
| | | Bridge Helix: 101-121 |
| | | RuvC-II: 132-175 |
| | | HNH: 215-247 |
| | | RuvC-III: 284-327 |
| 6 | sp\|G3ECR1\|CAS9_ | RuvC-I: 28-42 |
| | STRTR | Bridge Helix: 85-108 |
| | | Rec: 118-736 |
| | | RuvC-II: 750-799 |
| | | HNH: 864-896 |
| | | RuvC-III: 957-1019 |
| | | PAM Interaction (PI): 1119-1409 |
| 7 | sp\|J7RUA5\|CAS9_ | RuvC-I: 7-21 |
| | STAAU | Bridge Helix: 49-72 |
| | | Rec: 80-433 |
| | | RuvC-II: 445-493 |
| | | HNH: 553-585 |
| | | RuvC-III: 654-709 |
| | | PAM Interaction (PI): 789-1053 |
| 8 | Streptococcus_ | RuvC-I: 4-18 |
| | pyogenes_SF370 | Bridge Helix: 61-84 |
| | | Rec: 94-718 |
| | | RuvC-II: 725-774 |
| | | HNH: 833-865 |
| | | RuvC-III: 926-988 |
| | | PAM Interaction (PI): 1099-1365 |

### X Domains

In some embodiments, the IscB proteins comprise an X domain, e.g., at its N-terminal.

In certain embodiments, the X domain include the X domains in Table 1. Examples of the X domains also include any polypeptides a structural similarity and/or sequence similarity to a X domain described in the art. In some examples, the X domain may have an amino acid sequence that share at least 50%, at least 55%, at least 60%, at least 50%, at least 70%, at least 75%, at least 80%, at least 85%, at least 90%, at least 95%, at least 99%, or 100% sequence identity with X domains in Table 1.

In some examples, the X domain may be no more than 10, no more than 20, no more than 30, no more than 40, no more than 50, no more than 60, no more than 70, no more than 80, no more than 90, or no more than 100 amino acids in length. For example, the X domain may be no more than 50 amino acids in length, such as comprising 2 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

### Y Domain

In some embodiments, the IscB proteins comprise a Y domain, e.g., at its C-terminal.

In certain embodiments, the X domain include Y domains in Table 1. Examples of the Y domain also include any polypeptides a structural similarity and/or sequence similarity to a Y domain described in the art. In some examples, the Y domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with Y domains in Table 1.

### RuvC Domain

In some embodiments, the IscB proteins comprises at least one nuclease domain. In certain embodiments, the IscB proteins comprise at least two nuclease domains. In certain embodiments, the one or more nuclease domains are only active upon presence of a cofactor. In certain embodiments, the cofactor is Magnesium (Mg). In embodiments where more than one nuclease domain is present and the substrate is a double-strand polynucleotide, the nuclease domains each cleave a different strand of the double-strand polynucleotide. In certain embodiments, the nuclease domain is a RuvC domain.

The IscB proteins may comprise a RuvC domain. The RuvC domain may comprise multiple subdomains, e.g., RuvC-I, RuvC-II and RuvC-III. The subdomains may be separated by interval sequences on the amino acid sequence of the protein.

In certain embodiments, examples of the RuvC domain include those in Table 1. Examples of the RuvC domain also include any polypeptides a structural similarity and/or sequence similarity to a RuvC domain described in the art. For example, the RuvC domain may share a structural similarity and/or sequence similarity to a RuvC of Cas9. In some examples, the RuvC domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with RuvC domains in Table 1.

### Bridge Helix

The IscB proteins comprise abridge helix (BH) domain. The bridge helix domain refers to a helix and arginine rich polypeptide. The bridge helix domain may be located next to anyone of the amino acid domains in the nucleic-acid guided nuclease. In some embodiments, the bridge helix domain is next to a RuvC domain, e.g., next to RuvC-I, RuvC-II, or RuvC-III subdomain. In one example, the bridge helix domain is between a RuvC-1 and RuvC2 subdomains.

The bridge helix domain may be from 10 to 100, from 20 to 60, from 30 to 50, e.g., 35, 36, 37, 38, 39, 40, 41, 42, 43, 44, 45, 46 or 47, 48, 49, or 50 amino acids in length. Examples of bridge helix includes the polypeptide of amino acids 60-93 of the sequence of S. pyogenes Cas9.

In certain embodiments, examples of the BH domain include those in Table 1. Examples of the BH domain also include any polypeptides a structural similarity and/or sequence similarity to a BH domain described in the art. For example, the BH domain may share a structural similarity and/or sequence similarity to a BH domain of Cas9. In some examples, the BH domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with BH domains in Table 1.

### HNH Domain

The IscB proteins comprise an HNH domain. In certain embodiments, at least one nuclease domain shares a substantial structural similarity or sequence similarity to a HNH domain described in the art.

In some examples, the nucleic acid-guided nuclease comprises a HNH domain and a RuvC domain. In the cases where the RuvC domain comprises RuvC-I, RuvC-II, and RuvC-III domain, the HNH domain may be located between the Ruv C II and RuvC III subdomains of the RuvC domain.

In certain embodiments, examples of the HNH domain include those in Table 1. Examples of the HNH domain also include any polypeptides a structural similarity and/or sequence similarity to a HNH domain described in the art. For example, the HNH domain may share a structural similarity and/or sequence similarity to a HNH domain of Cas9. In some examples, the HNH domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with HNH domains in Table 1.

hRNA

In some examples, the IscB proteins capable of forming a complex with one or more hRNA molecules. The hRNA complex can comprise a guide sequence and a scaffold that interacts with the IscB polypeptide. An hRNA molecules may form a complex with an IscB polypeptide nuclease or IscB polypeptide and direct the complex to bind with a target sequence. In certain example embodiments, the hRNA molecule is a single molecule comprising a scaffold sequence and a spacer sequence. In certain example embodiments, the spacer is 5′ of the scaffold sequence. In certain example embodiments, the hRNA molecule may further comprise a conserved nucleic acid sequence between the scaffold and spacer portions.

As used herein, a heterologous hRNA molecule is an hRNA molecule that is not derived from the same species as the IscB polypeptide nuclease, or comprises a portion of the molecule, e.g., spacer, that is not derived from the same species as the IscB polypeptide nuclease, e.g., IscB protein. For example, a heterologous hRNA molecule of a IscB polypeptide nuclease derived from species A comprises a polynucleotide derived from a species different from species A, or an artificial polynucleotide.

### Zinc Finger Nucleases

In some embodiments, the polynucleotide is modified using a Zinc Finger nuclease or system thereof. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### TALE Nucleases

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂X₁₃)-X₁₋₃₃ or 34 or 35, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X₁₄₋₃₃ or 34 or 35)_{z}, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 9)
MDPIRSRTPSPARELLSGPQPDGVQPTADRGVSPPAGGP
LDGLPARRTMSRTRLPSPPAPSPAFSADSFSDLLRQFDPSLENTS
LFDSLPPFGAHHTEAATGEWDEVQSGLRAADAPPPTMRVAVTA
ARPPRAKPAPRRRAAQPSDASPAAQVDLRTLGYSQQQQEKIKP
KVRSTVAQHHEALVGHGFTHAHIVALSQHPAALGTVAVKYQD
MIAALPEATHEAIVGVGKQWSGARALEALLTVAGELRGPPLQL
DTGQLLKIAKRGGVTAVEAVHAWRNALTGAPLN

An exemplary amino acid sequence of a C-terminal capping region is:

(SEQ ID NO: 10)
RPALESIVAQLSRPDPALAALTNDHLVALACLGGRPAL
DAVKKGLPHAPALIKRTNRRIPERTSHRVADHAQVVRVLGFFQ
CHSHPAQAFDDAMTQFGMSRHGLLQLFRRVGVTELEARSGTLP
PASQRWDRILQASGMKRAKPSPTSTQTPDQASLHAFADSLERD
LDAPSPMHEGDQTRAS

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments the effector domain is an enhancer of transcription (i.e., an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated by reference.

### Sequences Related to Nucleus Targeting and Transportation

In some embodiments, one or more components (e.g., the Cas protein and/or deaminase, Zn Finger protein, TALE, or meganuclease) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO: 11) or PKKKRKVEAS (SEQ ID NO: 12); the NLS from nucleoplasmin (e.g., the nucleoplasmin bipartite NLS with the sequence KRPAATKKAGQAKKKK (SEQ ID NO: 13)); the c-myc NLS having the amino acid sequence PAAKRVKLD (SEQ ID NO: 14) or RQRRNELKRSP (SEQ ID NO: 15); the hRNPA1 M9 NLS having the sequence NQSSNFGPMKGGNFGGRSSGPYGGGGQYFAKPRNQGGY (SEQ ID NO: 16); the sequence RMRIZFKNKGKDTAELRRRRVEVSVELRKAKKDEQILKRRNV (SEQ ID NO: 17) of the IBB domain from importin-alpha; the sequences VSRKRPRP (SEQ ID NO: 18) and PPKKARED (SEQ ID NO: 19) of the myoma T protein; the sequence PQPKKKPL (SEQ ID NO: 20) of human p53; the sequence SALIKKKKKMAP (SEQ ID NO: 21) of mouse c-abl IV; the sequences DRLRR (SEQ ID NO: 22) and PKQKKRK (SEQ ID NO: 23) of the influenza virus NS1; the sequence RKLKKKIKKL (SEQ ID NO: 24) of the Hepatitis virus delta antigen; the sequence REKKKFLKRR (SEQ ID NO: 25) of the mouse Mxl protein; the sequence KRKGDEVDGVDEVAKKKSKK (SEQ ID NO: 26) of the human poly(ADP-ribose) polymerase; and the sequence RKCLQAGMNLEARKTKK (SEQ ID NO: 27) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein, or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In certain embodiments, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In certain embodiments, guides of the disclosure comprise specific binding sites (e.g. aptamers) for adapter proteins, which may be linked to or fused to an nucleotide deaminase or catalytic domain thereof. When such a guide forms a CRISPR complex (e.g., CRISPR-Cas protein binding to guide and target) the adapter proteins bind and, the nucleotide deaminase or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g., due to steric hindrance within the three dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively, or additionally, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### Templates

In some embodiments, the composition for engineering cells comprises a template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include sequence that corresponds to both, a first site on the target sequence that is cleaved in a first Cas protein mediated event, and a second site on the target sequence that is cleaved in a second Cas protein mediated event.

In certain embodiments, the template nucleic acid can include sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include sequence which, when integrated, results in: decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include sequence which results in: a change in sequence of 1, 2, 3, 4, 5, 6, 7, 8, 9, 10, 11, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 1 10+/−10, 120+/−10, 130+/−10, 140+/−10, 150+/−10, 160+/−10, 170+/−10, 1 80+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 1 10+/−20, 120+/−20, 130+/−20, 140+/−20, 150+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g., about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may be designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

In certain embodiments, a template nucleic acid for correcting a mutation may be designed for use with a homology-independent targeted integration system. Suzuki et al. describe in vivo genome editing via CRISPR/Cas9 mediated homology-independent targeted integration (2016, Nature 540:144-149). Schmid-Burgk, et al. describe use of the CRISPR-Cas9 system to introduce a double-strand break (DSB) at a user-defined genomic location and insertion of a universal donor DNA (Nat Commun. 2016 Jul. 28; 7:12338). Gao, et al. describe “Plug-and-Play Protein Modification Using Homology-Independent Universal Genome Engineering” (Neuron. 2019 Aug. 21; 103(4):583-597).

### RNAi

In some embodiments, the genetic modulating agents may be interfering RNAs. In certain embodiments, diseases caused by a dominant mutation in a gene is targeted by silencing the mutated gene using RNAi. In some cases, the nucleotide sequence may comprise coding sequence for one or more interfering RNAs. In certain examples, the nucleotide sequence may be interfering RNA (RNAi). As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e., although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

In certain embodiments, a modulating agent may comprise silencing one or more endogenous genes. As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g., about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA”, used interchangeably herein, are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Adoptive Cell Transfer

In certain embodiments, non-pathogenic Th1 cells are used to treat autoimmunity by adoptively transferring the cells to a subject in need thereof. In certain embodiments, non-pathogenic Th1 cells are generated by treating Th1 cells or naive CD4 T cells with an agent capable of decreasing expression of IL-23R. In certain embodiments, non-pathogenic Th1 cells are generated by treating Th1 cells or naive CD4 T cells with an agent capable of decreasing expression of one or more of CD160, GPR18, GZMB, ITGB1, CCR3, GZMA, IL22, ZFP36L2, ZBTB38, CD74, CCR5, MAP3K8, TNFSF8, IFITM1, NFKBIZ, FOSL2, CREM, CCDC85B, FOS, GPR183, S100A4, 1110008F13RIK, LSP1, LITAF, CD7, DUSP2, PLAC8, H1F0, S1PR1, NCF4, SMIM3, TESC, RBMS1, LPXN, TNFRSF9, PMM1, TOB2, IFNG, CD226, and CTSW. For example, Th1 cells can be modified ex vivo using a genetic modifying agent (e.g., CRISPR, RNAi). In certain embodiments, pathogenic Th1 cells are used in adoptive cell transfer to treat cancer (e.g., in vitro differentiated Th1 cells, as described herein). In certain embodiments, the Th1 cells are modified ex vivo to express a tumor antigen specific T cell receptor (TCR).

As used herein, “ACT”, “adoptive cell therapy” and “adoptive cell transfer” may be used interchangeably. In certain embodiments, Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Zacharakis et al., (2018) Nat Med. 2018 June; 24(6):724-730; Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Aspects of the invention involve the adoptive transfer of immune system cells, such as T cells, specific for selected antigens, such as tumor associated antigens or tumor specific neoantigens (see, e.g., Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144; and Rajasagi et al., 2014, Systematic identification of personal tumor-specific neoantigens in chronic lymphocytic leukemia. Blood. 2014 Jul. 17; 124(3):453-62).

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: MR1 (see, e.g., Crowther, et al., 2020, Genome-wide CRISPR-Cas9 screening reveals ubiquitous T cell cancer targeting via the monomorphic MHC class I-related protein MR1, Nature Immunology volume 21, pages178-185), B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar. 8; Berdeja J G, et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CAR T Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostase; Prostatic acid phosphatase (PAP); elongation factor 2 mutant (ELF2M); Insulin-like growth factor 1 receptor (IGF-1R); gplOO; BCR-ABL (breakpoint cluster region-Abelson); tyrosinase; New York esophageal squamous cell carcinoma 1 (NY-ESO-1); κ-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGalp(1-4)bDGlcp(1-1)Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis(Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac(2-3)bDGalp(1-4)bDGlcp(1-1)Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WTi); ETS translocation-variant gene 6, located on chromosome 12p (ETV6-AML); sperm protein 17 (SPA17); X Antigen Family, Member 1A (XAGEl); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p⁵³ mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLECi2A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); L1CAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190KD bcr-abl); Pml/RARa (promyelocytic leukaemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); CD70; and any combination thereof.

In certain embodiments, ACT includes co-transferring CD4+Th1 cells and CD8+CTLs to induce a synergistic antitumour response (see, e.g., Li et al., Adoptive cell therapy with CD4+T helper 1 cells and CD8+ cytotoxic T cells enhances complete rejection of an established tumour, leading to generation of endogenous memory responses to non-targeted tumour epitopes. Clin Transl Immunology. 2017 October; 6(10): e160).

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1; 23(9):2255-2266. doi: 10.1158/1078-0432.CCR-16-1300. Epub 2016 Nov. 4; Qasim et al., 2017, Molecular remission of infant B-ALL after infusion of universal TALEN gene-edited CAR T cells, Sci Transl Med. 2017 Jan. 25; 9(374); Legut, et al., 2018, CRISPR-mediated TCR replacement generates superior anticancer transgenic T cells. Blood, 131(3), 311-322; Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018; and Roth, T. L. Editing of Endogenous Genes in Cellular Immunotherapies. Curr Hematol Malig Rep 15, 235-240 (2020)). Cells may be edited using any CRISPR system and method of use thereof as described herein. CRISPR systems may be delivered to an immune cell by any method described herein. In preferred embodiments, cells are edited ex vivo and transferred to a subject in need thereof.

T cells can be obtained from a number of sources, including peripheral blood mononuclear cells (PBMC), bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets.

In one embodiment of the invention, the method further comprises expanding the numbers of T cells in the enriched cell population. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000-fold, or most preferably at least about 100,000-fold. The numbers of T cells may be expanded using any suitable method known in the art.

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In some embodiments, the disclosed immune cells may be delivered or administered into a cavity formed by the resection of tumor tissue (i.e., intracavity delivery) or directly into a tumor prior to resection (i.e., intratumoral delivery). In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

### Administration

In certain embodiments, the one or more agents are administered to a subject in a pharmaceutical composition. A “pharmaceutical composition” refers to a composition that usually contains an excipient, such as a pharmaceutically acceptable carrier that is conventional in the art and that is suitable for administration to cells or to a subject.

The pharmaceutical composition according to the present invention can, in one alternative, include a prodrug. When a pharmaceutical composition according to the present invention includes a prodrug, prodrugs and active metabolites of a compound may be identified using routine techniques known in the art. (See, e.g., Bertolini et al., J. Med. Chem., 40, 2011-2016 (1997); Shan et al., J. Pharm. Sci., 86 (7), 765-767; Bagshawe, Drug Dev. Res., 34, 220-230 (1995); Bodor, Advances in Drug Res., 13, 224-331 (1984); Bundgaard, Design of Prodrugs (Elsevier Press 1985); Larsen, Design and Application of Prodrugs, Drug Design and Development (Krogsgaard-Larsen et al., eds., Harwood Academic Publishers, 1991); Dear et al., J. Chromatogr. B, 748, 281-293 (2000); Spraul et al., J. Pharmaceutical & Biomedical Analysis, 10, 601-605 (1992); and Prox et al., Xenobiol., 3, 103-112 (1992)).

The term “pharmaceutically acceptable” as used throughout this specification is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilizers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavorings, aromatizers, thickeners, agents for achieving a depot effect, coatings, antifungal agents, preservatives, stabilizers, antioxidants, tonicity controlling agents, absorption delaying agents, and the like. The use of such media and agents for pharmaceutical active components is well known in the art. Such materials should be non-toxic and should not interfere with the activity of the cells or active components.

The precise nature of the carrier or excipient or other material will depend on the route of administration. For example, the composition may be in the form of a parenterally acceptable aqueous solution, which is pyrogen-free and has suitable pH, isotonicity and stability. For general principles in medicinal formulation, the reader is referred to Cell Therapy: Stem Cell Transplantation, Gene Therapy, and Cellular Immunotherapy, by G. Morstyn & W. Sheridan eds., Cambridge University Press, 1996; and Hematopoietic Stem Cell Therapy, E. D. Ball, J. Lister & P. Law, Churchill Livingstone, 2000.

The pharmaceutical composition can be applied parenterally, rectally, orally or topically. Preferably, the pharmaceutical composition may be used for intravenous, intramuscular, subcutaneous, peritoneal, peridural, rectal, nasal, pulmonary, mucosal, or oral application. In a preferred embodiment, the pharmaceutical composition according to the invention is intended to be used as an infusion. The skilled person will understand that compositions which are to be administered orally or topically will usually not comprise cells, although it may be envisioned for oral compositions to also comprise cells, for example when gastro-intestinal tract indications are treated. Each of the cells or active components (e.g., immunomodulants) as discussed herein may be administered by the same route or may be administered by a different route. By means of example, and without limitation, cells may be administered parenterally and other active components may be administered orally.

Liquid pharmaceutical compositions may generally include a liquid carrier such as water or a pharmaceutically acceptable aqueous solution. For example, physiological saline solution, tissue or cell culture media, dextrose or other saccharide solution or glycols such as ethylene glycol, propylene glycol or polyethylene glycol may be included.

The composition may include one or more cell protective molecules, cell regenerative molecules, growth factors, anti-apoptotic factors or factors that regulate gene expression in the cells. Such substances may render the cells independent of their environment.

Such pharmaceutical compositions may contain further components ensuring the viability of the cells therein. For example, the compositions may comprise a suitable buffer system (e.g., phosphate or carbonate buffer system) to achieve desirable pH, more usually near neutral pH, and may comprise sufficient salt to ensure isoosmotic conditions for the cells to prevent osmotic stress. For example, suitable solution for these purposes may be phosphate-buffered saline (PBS), sodium chloride solution, Ringer's Injection or Lactated Ringer's Injection, as known in the art. Further, the composition may comprise a carrier protein, e.g., albumin (e.g., bovine or human albumin), which may increase the viability of the cells.

Further suitably pharmaceutically acceptable carriers or additives are well known to those skilled in the art and for instance may be selected from proteins such as collagen or gelatine, carbohydrates such as starch, polysaccharides, sugars (dextrose, glucose and sucrose), cellulose derivatives like sodium or calcium carboxymethylcellulose, hydroxypropyl cellulose or hydroxypropylmethyl cellulose, pregeletanized starches, pectin agar, carrageenan, clays, hydrophilic gums (acacia gum, guar gum, arabic gum and xanthan gum), alginic acid, alginates, hyaluronic acid, polyglycolic and polylactic acid, dextran, pectins, synthetic polymers such as water-soluble acrylic polymer or polyvinylpyrrolidone, proteoglycans, calcium phosphate and the like.

In certain embodiments, a pharmaceutical cell preparation as taught herein may be administered in a form of liquid composition. In embodiments, the cells or pharmaceutical composition comprising such can be administered systemically, topically, within an organ or at a site of organ dysfunction or lesion.

Preferably, the pharmaceutical compositions may comprise a therapeutically effective amount of the specified immune cells and/or other active components (e.g., immunomodulants). The term “therapeutically effective amount” refers to an amount which can elicit a biological or medicinal response in a tissue, system, animal or human that is being sought by a researcher, veterinarian, medical doctor or other clinician, and in particular can prevent or alleviate one or more of the local or systemic symptoms or features of a disease or condition being treated.

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, PA (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

The medicaments of the invention are prepared in a manner known to those skilled in the art, for example, by means of conventional dissolving, lyophilizing, mixing, granulating or confectioning processes. Methods well known in the art for making formulations are found, for example, in Remington: The Science and Practice of Pharmacy, 20th ed., ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, New York.

Administration of medicaments of the invention may be by any suitable means that results in a compound concentration that is effective for treating or inhibiting (e.g., by delaying) the development of a disease. The compound is admixed with a suitable carrier substance, e.g., a pharmaceutically acceptable excipient that preserves the therapeutic properties of the compound with which it is administered. One exemplary pharmaceutically acceptable excipient is physiological saline. The suitable carrier substance is generally present in an amount of 1-95% by weight of the total weight of the medicament. The medicament may be provided in a dosage form that is suitable for administration. Thus, the medicament may be in form of, e.g., tablets, capsules, pills, powders, granulates, suspensions, emulsions, solutions, gels including hydrogels, pastes, ointments, creams, plasters, drenches, delivery devices, injectables, implants, sprays, or aerosols.

Administration can be systemic or local. In addition, it may be advantageous to administer the composition into the central nervous system by any suitable route, including intraventricular and intrathecal injection. Pulmonary administration may also be employed by use of an inhaler or nebulizer, and formulation with an aerosolizing agent. It may also be desirable to administer the agent locally to the area in need of treatment; this may be achieved by, for example, and not by way of limitation, local infusion during surgery, topical application, by injection, by means of a catheter, by means of a suppository, or by means of an implant.

Various delivery systems are known and can be used to administer the pharmacological compositions including, but not limited to, encapsulation in liposomes, microparticles, microcapsules; minicells; polymers; capsules; tablets; and the like. In one embodiment, the agent may be delivered in a vesicle, in particular a liposome. In a liposome, the agent is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,837,028 and 4,737,323. In yet another embodiment, the pharmacological compositions can be delivered in a controlled release system including, but not limited to: a delivery pump (See, for example, Saudek, et al., New Engl. J. Med. 321: 574 (1989) and a semi-permeable polymeric material (See, for example, Howard, et al., J. Neurosurg. 71: 105 (1989)). Additionally, the controlled release system can be placed in proximity of the therapeutic target (e.g., a tumor), thus requiring only a fraction of the systemic dose. See, for example, Goodson, In: Medical Applications of Controlled Release, 1984. (CRC Press, Boca Raton, Fla.).

The amount of the agents which will be effective in the treatment of a particular disorder or condition will depend on the nature of the disorder or condition, and may be determined by standard clinical techniques by those of skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of the agent with which to treat each individual patient. In certain embodiments, the attending physician will administer low doses of the agent and observe the patient's response. Larger doses of the agent may be administered until the optimal therapeutic effect is obtained for the patient, and at that point the dosage is not increased further. Effective doses may be extrapolated from dose-response curves derived from in vitro or animal model test systems. Ultimately the attending physician will decide on the appropriate duration of therapy using compositions of the present invention. Dosage will also vary according to the age, weight and response of the individual patient.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection.

### Diagnostic Methods

In certain embodiments, detecting pathogenic Th1 cells or a pathogenic Th1 cell immune response can be used to detect Th1 mediated autoimmune diseases. In certain embodiments, detecting pathogenic Th1 cells or a pathogenic Th1 cell immune response can be used to determine whether a treatment is effective in a subject suffering from a disease. In certain embodiments, the disease is an autoimmune and the treatment is any treatment described herein or currently used for treatment. In certain embodiments, a treatment is effective in a subject suffering from an autoimmune disease if the treatment decreases pathogenic Th1 cells or a pathogenic signature described herein. In certain embodiments, the treatment is any treatment as described herein (e.g., a CD160, GPR18 inhibitors). In certain embodiments, the treatment is an IL-23 antagonist. In certain embodiments, the treatment is an IL-12 and IL-23 antagonist. Non-limiting IL-23 antagonists that are approved for use and whose effectiveness can be monitored include the monoclonal antibodies ustekinumab, guselkumab, risankizumab, and tildrakizumab. Ustekinumab targets IL-23 and IL-12 through their shared p40 subunit (see, e.g., Cingoz O. Ustekinumab. MAbs. 2009; 1(3):216-221. doi:10.4161/mabs.1.3.8593). Guselkumab, risankizumab, and tildrakizumab are selective IL-23 inhibitors, which are inhibitors of p19 of IL-23 (see, e.g., Gottlieb A B, Saure D, Wilhelm S, et al. Indirect comparisons of ixekizumab versus three interleukin-23 p19 inhibitors in patients with moderate-to-severe plaque psoriasis—efficacy findings up to week 12. J Dermatolog Treat. 2020; 1-8).

In certain embodiments, an autoimmune response can be detected by detecting pathogenic Th1 cells or non-pathogenic Th1 cells. In certain embodiments, pathogenic Th1 cells can be detected by detecting Th1 cells that express one or markers described herein. For example, Th1 cells that express IL-23R or one or more of IL23R, CD160, GPR18, GZMB, ITGB1, CCR3, GZMA, IL22, ZFP36L2, ZBTB38, CD74, CCR5, MAP3K8, TNFSF8, IFITM1, NFKBIZ, FOSL2, CREM, CCDC85B, FOS, GPR183, S100A4, 1110008F13RIK, LSP1, LITAF, CD7, DUSP2, PLAC8, H1F0, S1PR1, NCF4, SMIM3, TESC, RBMS1, LPXN, TNFRSF9, PMM1, TOB2, IFNG, CD226, and CTSW or a gene program selected from cluster 2 or cluster 9. In certain embodiments, pathogenic Th1 cells are increased. In certain embodiments, non-pathogenic Th1 cells can be detected by detecting Th1 cells expressing a program characterized by cluster 7 genes. In certain embodiments, non-pathogenic Th1 cells are decreased.

The invention provides biomarkers (e.g., phenotype specific or cell type) for the identification, diagnosis, prognosis and manipulation of cell properties, for use in a variety of diagnostic and/or therapeutic indications. Biomarkers in the context of the present invention encompasses, without limitation nucleic acids, proteins, reaction products, and metabolites, together with their polymorphisms, mutations, variants, modifications, subunits, fragments, and other analytes or sample-derived measures. In certain embodiments, biomarkers include the signature genes or signature gene products, and/or cells as described herein.

Biomarkers are useful in methods of diagnosing, prognosing and/or staging an immune response in a subject by detecting a first level of expression, activity and/or function of one or more biomarker and comparing the detected level to a control of level wherein a difference in the detected level and the control level indicates that the presence of an immune response in the subject.

The terms “diagnosis” and “monitoring” are commonplace and well-understood in medical practice. By means of further explanation and without limitation the term “diagnosis” generally refers to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

The biomarkers of the present invention are useful in methods of identifying patient populations at risk or suffering from an immune response based on a detected level of expression, activity and/or function of one or more biomarkers. These biomarkers are also useful in monitoring subjects undergoing treatments and therapies for suitable or aberrant response(s) to determine efficaciousness of the treatment or therapy and for selecting or modifying therapies and treatments that would be efficacious in treating, delaying the progression of or otherwise ameliorating a symptom. The biomarkers provided herein are useful for selecting a group of patients at a specific state of a disease with accuracy that facilitates selection of treatments.

The term “monitoring” generally refers to the follow-up of a disease or a condition in a subject for any changes which may occur over time.

The terms also encompass prediction of a disease. The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

Suitably, an altered quantity or phenotype of the immune cells in the subject compared to a control subject having normal immune status or not having a disease comprising an immune component indicates that the subject has an impaired immune status or has a disease comprising an immune component or would benefit from an immune therapy.

Hence, the methods may rely on comparing the quantity of immune cell populations, biomarkers, or gene or gene product signatures measured in samples from patients with reference values, wherein said reference values represent known predictions, diagnoses and/or prognoses of diseases or conditions as taught herein.

For example, distinct reference values may represent the prediction of a risk (e.g., an abnormally elevated risk) of having a given disease or condition as taught herein vs. the prediction of no or normal risk of having said disease or condition. In another example, distinct reference values may represent predictions of differing degrees of risk of having such disease or condition.

In a further example, distinct reference values can represent the diagnosis of a given disease or condition as taught herein vs. the diagnosis of no such disease or condition (such as, e.g., the diagnosis of healthy, or recovered from said disease or condition, etc.). In another example, distinct reference values may represent the diagnosis of such disease or condition of varying severity.

In yet another example, distinct reference values may represent a good prognosis for a given disease or condition as taught herein vs. a poor prognosis for said disease or condition. In a further example, distinct reference values may represent varyingly favourable or unfavourable prognoses for such disease or condition.

Such comparison may generally include any means to determine the presence or absence of at least one difference and optionally of the size of such difference between values being compared. A comparison may include a visual inspection, an arithmetical or statistical comparison of measurements. Such statistical comparisons include, but are not limited to, applying a rule.

Reference values may be established according to known procedures previously employed for other cell populations, biomarkers and gene or gene product signatures. For example, a reference value may be established in an individual or a population of individuals characterised by a particular diagnosis, prediction and/or prognosis of said disease or condition (i.e., for whom said diagnosis, prediction and/or prognosis of the disease or condition holds true). Such population may comprise without limitation 2 or more, 10 or more, 100 or more, or even several hundred or more individuals.

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or 1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-knownper se, such as positive predictive value (PPV), negative predictive value (NPV), positive likelihood ratio (LR+), negative likelihood ratio (LR−), Youden index, or similar.

In one embodiment, the signature genes, biomarkers, and/or cells may be detected or isolated by immunofluorescence, immunohistochemistry (IHC), fluorescence activated cell sorting (FACS), mass spectrometry (MS), mass cytometry (CyTOF), RNA-seq, single cell RNA-seq (described further herein), quantitative RT-PCR, single cell qPCR, FISH, RNA-FISH, MERFISH (multiplex (in situ) RNA FISH) and/or by in situ hybridization. Other methods including absorbance assays and colorimetric assays are known in the art and may be used herein. detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25).

In certain embodiments, a tissue sample may be obtained and analyzed for specific cell markers (IHC) or specific transcripts (e.g., RNA-FISH). Tissue samples for diagnosis, prognosis or detecting may be obtained by endoscopy. In one embodiment, a sample may be obtained by endoscopy and analyzed by FACS. As used herein, “endoscopy” refers to a procedure that uses an endoscope to examine the interior of a hollow organ or cavity of the body. The endoscope may include a camera and a light source. The endoscope may include tools for dissection or for obtaining a biological sample.

The present invention also may comprise a kit with a detection reagent that binds to one or more biomarkers or can be used to detect one or more biomarkers.

MS methods

Biomarker detection may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

### Immunoassays

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65 C for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B.V. (1993) and Kricka, “Nonisotopic DNA Probe Techniques”, Academic Press, San Diego, Calif. (1992).

### Single Cell Sequencing

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. Jan; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; International Patent Application No. PCT/US2016/059239, published as WO2017164936 on Sep. 28, 2017; International Patent Application No. PCT/US2018/060860, published as WO/2019/094984 on May 16, 2019; International Patent Application No. PCT/US2019/055894, published as WO/2020/077236 on Apr. 16, 2020; and Drokhlyansky, et al., “The enteric nervous system of the human and mouse colon at a single-cell resolution,” bioRxiv 746743; doi: doi.org/10.1101/746743, which are herein incorporated by reference in their entirety.

### Screening

A further aspect of the invention relates to a method for identifying an agent capable of modulating one or more phenotypic aspects of a pathogenic Th1 cell or cell population comprising Th1 cells. The Th1 cell or population comprising Th1 cells can be Th1 cells differentiated in vitro as described herein. In certain embodiments, the method comprises a) applying a candidate agent to the cell or cell population; b) detecting modulation of one or more phenotypic aspects of the cell or cell population by the candidate agent (e.g., expression of one or more genes or program selected from cluster 2, 9 or 7 in Table 3), thereby identifying the agent. The phenotypic aspects of the cell or cell population that is modulated may be one or more genes or biological program specific to a Th1 cell phenotype (e.g., an inflammatory phenotype or suppressive immune phenotype). In certain embodiments, steps can include administering candidate modulating agents to cells, detecting identified cell (sub)populations for changes in signatures, or identifying relative changes in cell (sub) populations which may comprise detecting relative abundance of particular gene signatures.

The term “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, one or more desired phenotypic aspects of an immune cell or immune cell population may be modulated without substantially altering other (unintended, undesired) phenotypic aspect(s).

The term “agent” broadly encompasses any condition, substance or agent capable of modulating one or more phenotypic aspects of a cell or cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a cell or cell population as disclosed herein in a method comprising applying the candidate agent to the cell or cell population (e.g., exposing the cell or cell population to the candidate agent or contacting the cell or cell population with the candidate agent) and observing whether the desired modulation takes place.

Agents may include any potential class of biologically active conditions, substances or agents, such as for instance antibodies, proteins, peptides, nucleic acids, oligonucleotides, small molecules, or combinations thereof, as described herein.

The methods of phenotypic analysis can be utilized for evaluating environmental stress and/or state, for screening of chemical libraries, and to screen or identify structural, syntenic, genomic, and/or organism and species variations. For example, a culture of cells, can be exposed to an environmental stress, such as but not limited to heat shock, osmolarity, hypoxia, cold, oxidative stress, radiation, starvation, a chemical (for example a therapeutic agent or potential therapeutic agent) and the like. After the stress is applied, a representative sample can be subjected to analysis, for example at various time points, and compared to a control, such as a sample from an organism or cell, for example a cell from an organism, or a standard value. By exposing cells, or fractions thereof, tissues, or even whole animals, to different members of the chemical libraries, and performing the methods described herein, different members of a chemical library can be screened for their effect on immune phenotypes thereof simultaneously in a relatively short amount of time, for example using a high throughput method.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

In certain embodiments, the present invention provides for gene signature screening. The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature was the proxy for a phenotype of interest, it could be used to find small molecules that effect that phenotype without knowledge of a validated drug target. The signatures or biological programs of the present invention may be used to screen for drugs that reduce the signature or biological program in cells as described herein. The signature or biological program may be used for GE-HTS. In certain embodiments, pharmacological screens may be used to identify drugs that are selectively toxic to cells having a signature.

The Connectivity Map (cmap) is a collection of genome-wide transcriptional expression data from cultured human cells treated with bioactive small molecules and simple pattern-matching algorithms that together enable the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science.1132939; and Lamb, J., The Connectivity Map: a new tool for biomedical research. Nature Reviews Cancer January 2007: Vol. 7, pp. 54-60). In certain embodiments, Cmap can be used to screen for small molecules capable of modulating a signature or biological program of the present invention in silico.

Further embodiments are illustrated in the following Examples which are given for illustrative purposes only and are not intended to limit the scope of the invention.

### EXAMPLES

### Example 1—In Vitro Differentiation of Naïve T Cells with IL-12 and IL-21 Induces IL-23R⁺ Th1 Cells

Although it has been shown in vivo that IFN-γ⁺ T cells can express IL-23R, these IFN-γ-producing cells were identified as transdifferentiated Th17 cells using a fate-mapping approach¹⁹. In fact, chronic stimulation of Th17 cells with IL-23 was shown to increase IFN-γ production from Th17 cells and to inhibit IL-17 production¹⁹. However, whether there are bona fide Th1 cells that express IL-23R and thus may be impacted by IL-23 signaling has not been addressed. It has been long established that naïve T cells differentiated in vitro with IL-12 to become Th1 cells express IL-12R but are not known to express IL-23R²⁰. Therefore, Applicants sought to identify whether there are bonafide Th1 cells that express IL-23R and undertook a screen to identify a cytokine that in conjunction with IL-12 would efficiently induce IL-23R in Th1 cells in vitro. Th1 cells express IFN-γ, IL-2, lymphotoxin (LT) and the master transcription factor T-bet but do not produce IL-17A, the signature cytokine of Th17 cells^{20,21}. Screening different cytokine conditions (FIG. 5), Applicants discovered that culturing naïve CD4+ T cells with IL-12+IL-21 for 5 days induces strong expression of IL-23R, together with IFN-γ expression, the signature cytokine of Th1 cells (FIG. 1a and FIG. 5). The expression of IL-23R in Th1 cells reached similar levels as in pathogenic Th17 cells that were differentiated with the cytokine combination IL-1β+IL-6+IL-23 (FIG. 1a)⁸. When Applicants further added IL-23 to the differentiation condition (IL-12+IL-21+IL-23), it slightly increased the expression of Il23r compared to IL-12+IL-21, and it is known that IL-23 can increase the expression of its own receptor (FIG. 1a and FIG. 5). Differentiation of naïve T cells with IL-12 or IL-18 alone induced minimal expression of IL-23R (FIG. 5). Addition of IL-23 together with IL-12 did also induce Il23r expression, however, the induction of IL-23R and IFN-γ expression was more pronounced with IL-12+IL-21. Applicants also measured the expression of Il23r, Ifng, III7a, Tbx21, Rorc and Stat3 in these in vitro differentiated T cells and could confirm the induction of Il23r together with a Th1 cell transcriptional program (FIG. 1b). In summary, Applicants demonstrate that IL-12+IL-21 strongly induces IL-23R expression in Th1 cells in vitro.

### Example 2—Single-Cell RNA-Sequencing Identifies Genes in Th1 Cells that are Expressed in an IL-23R Dependent Manner

It is known that IL-23R signaling induces a set of genes in Th17 cells that makes them pathogenic, therefore, Applicants studied the impact of IL-23R signaling on Th1 cells. Applicants took advantage of a previously described reporter allele for Il23r expression and chose a two-pronged approach: (1) Applicants differentiated naïve T cells from reporter mice that carry an eGFP reporter in their endogenous IL-23R locus (Il23r^{eGFP/wt}) with IL-12+IL-21+IL-23 for 96 hours. Applicants then isolated IL-23R⁺ (i.e., eGFP⁺) and IL-23R⁻ (i.e., eGFP⁻) cells and conducted scRNAseq of the two populations using Smart-seq2 protocol^{22,23}. Notably, Applicants added IL-23 to the cytokine combination in this experiment in order to enhance signaling through IL-23R and thus better identify its role in Th1 cells. (2) Secondly, Applicants differentiated naïve T cells from IL-23R-deficient mice (Il23r^{eGFP/eGFP}) with the same set of cytokines and sorted eGFP⁺ vs. eGFP⁻ cells for the expression analysis by scRNAseq (FIG. 1c).

Applicants analyzed the expression of signature Th1 and Th17 cell genes in these in vitro differentiated cells and confirmed at the single-cell level that IFN-γ-expressing Th1 cells co-express IL-23R but do not express Th17 cell signature genes such as Il17a, Il17f and Ccr6. In addition to Ifng, the cells prominently expressed Th1 cell signature genes including Tbx21 and Cxcr3 (FIG. 1d and FIG. 6). A low frequency of cells expressed Rorc, encoding the transcription factor RORγt associated with Th17 cell differentiation, but the expression of Rorc was minimal when compared to the Th1 cell master transcription factor Tbx21 (FIG. 1d and FIG. 6).

Comparing the IL-23R⁺ vs. the IL-23R⁻ subsets from the wildtype cells (Il23r^{eGFP/wt}) provided a way to identify genes that may be co-expressed with IL-23R (FIGS. 1e and f). Here, Applicants identified a set of 528 differentially expressed (DE) genes whose expression in Th1 cells is positively or negatively associated with the presence of IL-23R and may be involved in pathogenicity of Th1 cells similar to how IL-23R signaling promotes pathogenicity of Th17 cells^{13,24}. Indeed, the set of genes that were co-expressed with IL-23R included critical effector molecules and transcription factors including Il18r1 and Stat5a (FIG. 1e and Table 1).

Strikingly, Applicants found that the number of differentially expressed genes between the eGFP⁺ and eGFP⁻ populations in knockout cells (Il23r^{eGFP/eGFP}) was substantially lower than the IL-23R⁺ vs. IL-23R⁻ comparison in wildtype cells (129 vs 528 at FDR<0.1 with only 33 genes overlapping). These eGFP⁺ knockout cells would reflect differentiation in response to IL-12 plus IL-21 in the absence of IL-23R signaling. This finding suggests that the majority of genes Applicants identified in the wildtype cells as being differentially expressed between the IL-23R⁺ and IL-23R⁻ populations are more likely to be directly affected by IL-23R signaling.

Applicants then undertook a combined analysis of the expression profiles from both wildtype and IL23R-deficient cells to better distinguish genes directly regulated by IL-23R signaling in Th1 cells (FIG. 1g). To this end, Applicants combined the datasets and incorporated model terms for the presence of eGFP, the genotype (wildtype or IL-23R-deficient), and the interaction of eGFP and genotype (see methods for full details). By testing the interaction term, Applicants sought genes whose expression difference between eGFP⁺ and eGFP⁻ cells was dependent on the genotype. In this manner Applicants identified altogether 219 genes where the presence of wildtype IL-23R was associated with an increase in expression in eGFP⁺ cells compared to eGFP⁻ cells. Of those, Dusp2, Gpr18, Trim27, Itgb1, Gzmb, Pde4b, Plek, Thap11, Panx1, Peli1, Rac1 and Lamp1(CD107a) appeared of particular interest as novel candidate genes dependent on IL-23R signaling and potentially important for pathogenicity of Th1 cells (FIG. 1g and Table 1). Some of these genes have been implicated previously in autoimmune inflammation, such as Panx1²⁵ and Rac1²⁶. The finding that these genes are regulated in an IL-23R-dependent manner suggests an important function in Th1 cells.

In summary, in addition to pathogenic Th17 cells, Applicants find that Th1 cells can be induced to express IL-23 receptor in vitro through culture with IL-12+IL-21 and that the single-cell transcriptomic profile corresponds to that of Th1 cells. Through this process, Applicants have further identified a unique gene set associated with IL-23R signaling in Th1 cells.

### Example 3—IL-23R⁺ Th1 and IL-23R+Th17 Cells Share Common Gene Signatures but Also Display Subset-Specific Features

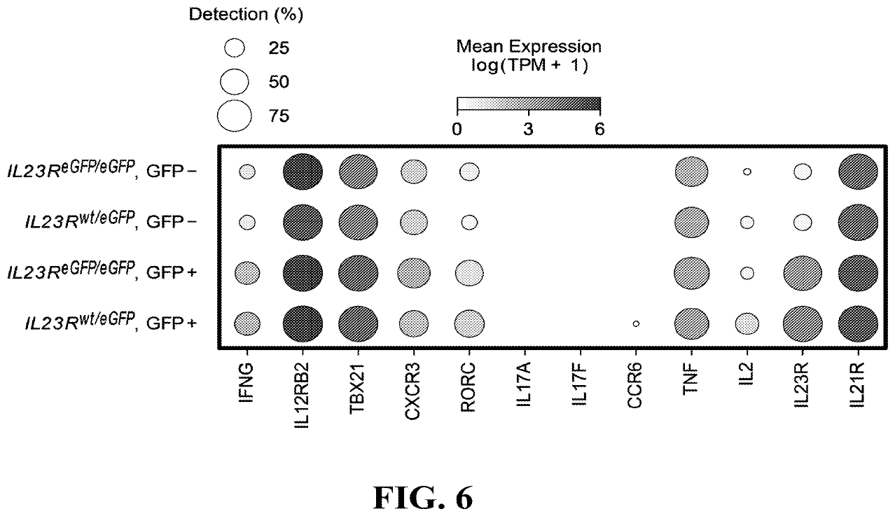

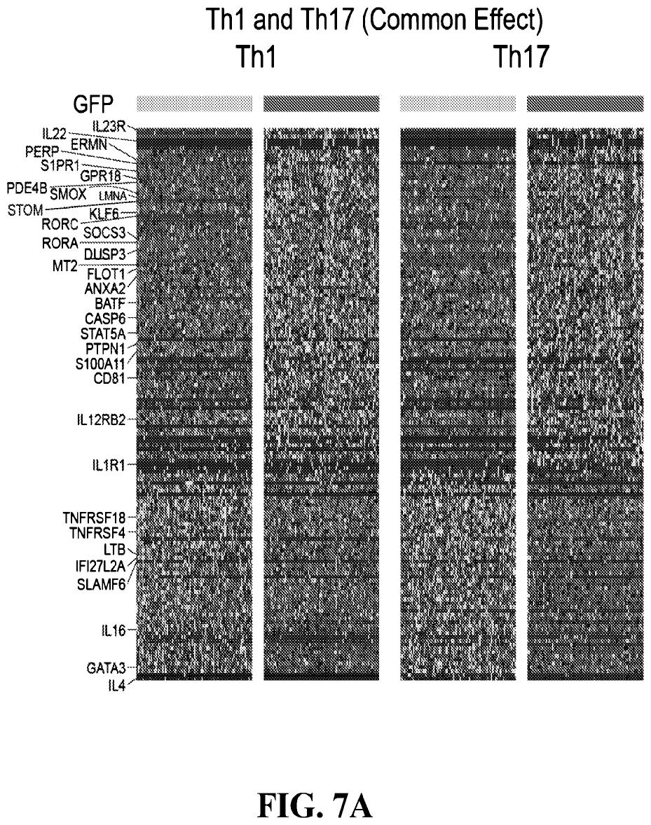

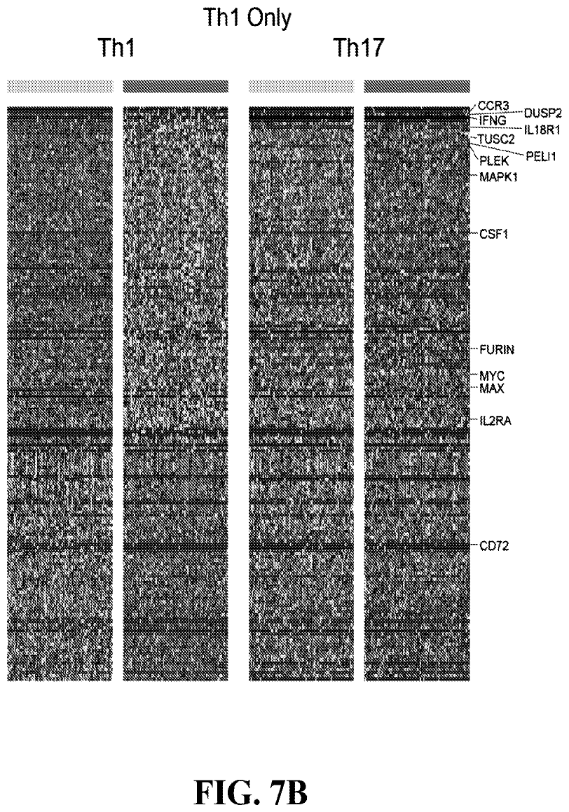

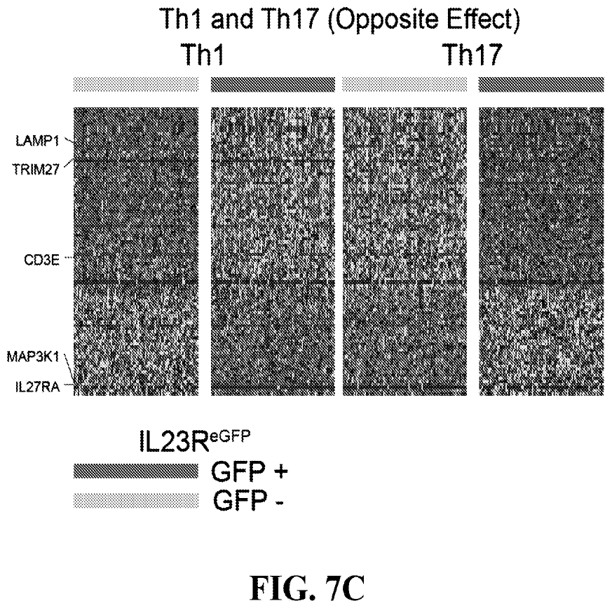

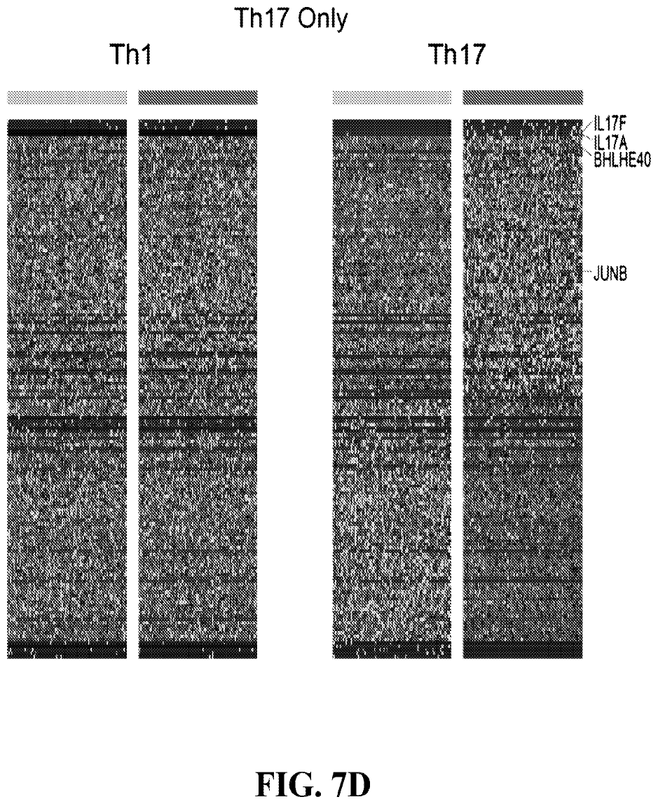

To investigate whether IL-23R⁺ Th1 and IL-23R⁺ Th17 cells share common features, Applicants differentiated pathogenic Th17 cells with IL-1β+IL-6+IL-23 and sorted IL-23R⁺ (i.e., eGFP⁺) and IL-23R⁻ (i.e. eGFP⁻) cells for single-cell analysis comparing their transcriptional profiles with those of Th1 cells described above. Interestingly, Applicants find a set of 147 genes whose expression in IL-23R⁺ and IL-23R⁻ cells is shared by Th1 and Th17 cells (FIG. 7a and Table 1). Among the genes highly expressed in both IL-23R⁺ Th1 and Th17 cells, Applicants find Il22, which has been identified previously as a core component of the pathogenicity signature of Th17 cells^{9,11}. Some genes (179) were expressed only in Th1 cells differentially between IL-23R⁺ and IL-23R⁻ cells such as Il18r1 and Ifng (FIG. 7b and Table 1). Interestingly, 93 genes showed the exact opposite profile in Th1 and Th17 cells, one of which was Il27ra which was highly expressed in IL-23R⁻ Th1 cells (FIG. 7c and Table 1). Finally, Il17a and Il17f were prominently expressed in IL-23R⁺ cells in Th17 cells only and were among 158 genes that were differentially expressed between IL-23R⁺ cells and IL-23R⁻ cells only in Th17 cells (FIG. 7d and Table 1). These results suggest that IL-23R induces a common Th1/Th17 pathogenicity expression profile but also induces a distinct set of genes in Th1 and Th17 cells that likely contribute to their pathogenicity to exert lineage-specific phenotype and function.

### Example 4—IL-23R Deficiency Protects from Th1 Cell Adoptive Transfer Colitis

GWAS analysis has implicated IL-23R as a major risk gene for IBD⁵, but the actual mechanism by which IL-23R signaling promotes the disease has not been fully elucidated. It has been known that naïve T cells that lack IL-23R lose the ability to induce adoptive transfer colitis, presumably by not developing Th17 cells and promoting differentiation of FoxP3+Tregs^{3,14,18}. However, as bonafide Th1 cells elicit adoptive transfer colitis⁷, Applicants assessed the issue of whether IL-23R has a role in these pathogenic Th1 cells to induce colitis. To test the contribution of IL-23R to the pathogenicity of Th1 cells in vivo, Applicants differentiated wildtype (I123^{eGFP/wt}) and IL-23R-knockout (Il23r^{eGFP/eGFP}) Th1 cells in vitro with IL-12+IL-21+IL-23 (see methods for details) and then adoptively transferred them to RAG1^{-/-} recipients (FIG. 2a). Strikingly, Applicants found that Th1 cells that lack IL-23R do not efficiently induce severe colitis, as determined by the histopathological exam of the colon (FIG. 2b). The results therefore establish an important function of IL-23R in the pathogenicity of Th1 cells and their ability to induce colitis.

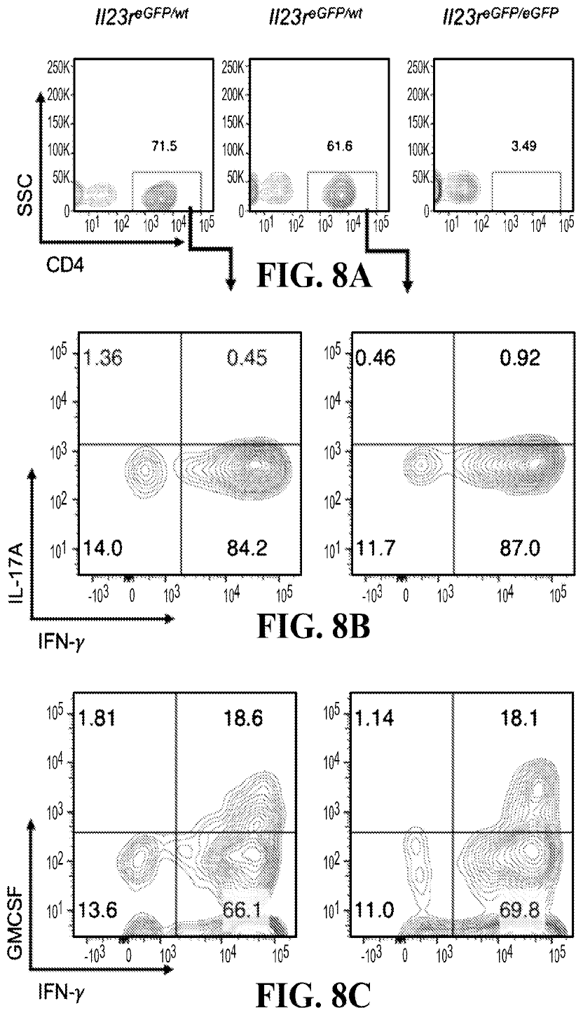

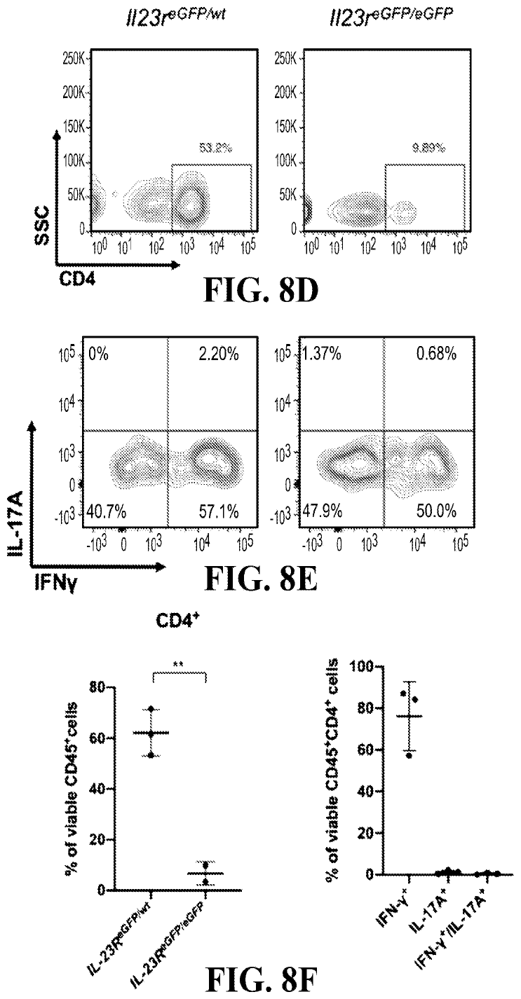

To test whether the transferred Th1 cells produced IFN-γ or had transdifferentiated into IL-17A-producing Th17 cells, Applicants isolated colonic intraepithelial lymphocytes (IEL) as well as lamina propria lymphocytes (LPL) and tested these cells for the production of IFN-γ, IL-17A and GM-CSF. Animals that received wildtype (Il23r^{eGFP/wt}) Th1 cells had a dramatically increased frequency of CD4⁺ intraepithelial lymphocytes and lamina propria lymphocytes, up to 60-70% of the entire CD45⁺ population vs. 4%-9% in recipients of knockout (Il23r^{eGFP/eGFP}) Th1 cells, pointing to the ongoing inflammatory process (FIGS. 8a, d and f). Interestingly, Applicants found that in the animals studied here, the CD4⁺ T cells had largely maintained their production of IFN-γ and had not significantly deviated into IL-17A-producing cells (FIGS. 8b, e and g). Furthermore, a sizable frequency of IFN-γ-producing cells in the colon also produced GM-CSF (FIG. 8c). It is known that both in the EAE model and the colitis model Applicants used here, Th17 cells show extensive plasticity and become IL-17A⁺/IFN-γ⁺ double positive^{7,19}. The results suggest that at least in these settings, Th1 cells were not plastic and retained a Th1 cell phenotype. Taken together, these data suggest that colitogenic Th1 cells require IL-23R expression in order to induce disease in vivo similar to Th17 cells and that these cells do not transdifferentiate towards a Th17 phenotype. Therefore, pathogenicity mediated by IL-23R signaling is separable and not limited to Th17 cells in vivo.

### Example 5—Single-Cell RNA-Sequencing Reveals Transcriptional Signatures of Colitogenic Intestinal Lymphocytes and their Dependence on IL-23R Signaling

To gain a better understanding of the transcriptional programs that may be driven by IL-23R in transferred Th1 cells and therefore associated with pathogenicity, Applicants isolated CD45⁺CD4⁺ infiltrating T cells from the intestinal mucosa (both the intraepithelial lymphocytes, IEL, and the lamina propria lymphocytes, LPL) from animals that received either wildtype or IL-23R KO cells by fluorescence-activated cell sorting (FACS). Applicants then performed massively parallel single-cell transcriptional profiling by 10×technology of 32,763 cells (FIG. 2a).

The cells primarily cluster depending on which tissue they were isolated from, with splenic CD4⁺ T cells segregating from cells of intestinal origin (FIG. 2c). These results confirm previous findings that T cells acquire distinct transcriptional profiles depending on whether they are isolated from secondary lymphoid organs such as the spleen or reside in tissues such as the intestine²⁷. Applicants find the expression of 1224 genes was strongly associated with the tissue of origin (FDR<0.1, |log FC|>0.5), whereas other genes showed a broader expression in cells of all tissue origins (FIG. 2d and Table 2). For instance, CD69 has been proposed as a marker of tissue residency in T cells including in T_{RM} (tissue resident memory) cells and the precise tissue specific expression of Rgs1 suggests that it could be involved in trafficking and tissue residency as well, in particular in the intestinal mucosa²⁸. Klf2, a transcription factor that is important for the expression of CD62L and S1pr1, and which was shown to direct lymphocyte trafficking was highly upregulated in splenic cells suggesting an antagonistic function to CD69 and potentially Rgs1 as well²⁹. Interestingly, the chemokine Cxcl10 was particularly highly expressed in T cells within the LPL but not IEL (FIG. 2d), suggesting a very specific autocrine/paracrine regulation, in particular since the contribution of Cxc110 to the trafficking and retention of pro-inflammatory Th1 cells expressing the corresponding receptor Cxcr3 has been previously identified^{30,31}. Overall, multiple chemokine-related transcripts were expressed in a compartment-specific fashion and therefore likely contribute to the trafficking of Th1 cells to the intestine (FIG. 2d).

In terms of their splenic transcriptional profiles, it appeared that the wildtype Th1 cells (Il23r^{eGFP/wt}) and the knockout cells (Il23r^{eGFP/eGFP}) isolated from recipient mice were rather uniformly distributed (FIG. 2e). Strikingly, the wildtype and KO cells appeared to instead segregate within the intestinal tissues, suggesting that Th1 cells from the wildtype vs. IL-23R KO mice may attain transcriptionally different expression profiles when they infiltrate the intestine (FIG. 2e). Applicants then asked which genes would be differentially expressed between wildtype and KO cells across the four analyzed tissues (spleen, LPL small intestine (SI), LPL colon and IEL colon) and identified between 84 and 190 genes per tissue (FDR<0.1 and |log FC|>0.5) (Table 2). Applicants identified genes such as Gpr18, Cd160 and Zfp36l2 being differentially expressed dependent on IL-23R signaling in all or most tissues in vivo (FIG. 2f). Interestingly, the chemokine Ccl5 (RANTES), a ligand of Ccr5, exhibited a clear IL-23R dependent expression within the intestine only, suggesting that the regulation of Ccl5 is particularly relevant within the tissue and that IL-23R might contribute to pathogenicity by enabling inflammatory cells to migrate to tissues. Cxcl10 did not show such IL-23R dependent regulation (FIG. 2f). Taken together, the observations support the notion that IL-23R signaling affects the tissue-specific transcriptional expression of novel genes relevant to intestinal inflammation.

### Example 6—IL-23R Drives the Expansion of Highly Inflammatory and Colitogenic Th1 Cells in the Lamina Propria as Uncovered by scRNAseq

Lymphocytes residing within the lamina propria exert a critical contribution to elicit intestinal inflammation³². Therefore, Applicants decided to focus on these samples (both from the colon and small intestine) in wildtype (Il23r^{eGFP/wt}) and IL-23R KO (Il23r^{eGFPeGFP}) cells (FIG. 3a). Applicants partitioned all cells into clusters and identified a total of 12 distinct clusters with characteristic transcriptional profiles within the LPL (FIG. 3b). Applicants then sought to identify clusters consisting of cells with a highly pro-inflammatory and colitogenic gene signature that may be responsible for mediating colitis. Importantly, Applicants noted differential contribution of wildtype or KO cells to some clusters, especially clusters 2, 8, 9 and 10 were dominated by cells from wildtype animals (Il23r^{eGFP/wt}) whereas clusters 5 and 7 were dominated by KO cells (Il23r^{eGPF/eGFP}) (FIG. 3c). Based on their transcriptional profiles, Applicants found that clusters 2 and 9 consisted of cells that exhibited a highly colitogenic gene signature and may have the ability to drive intestinal inflammation which will be highlighted in detail below (FIG. 3d).

Applicants found that the characterizing genes of cluster 9 (one vs. all comparison, FDR<0.1, |log FC|>0.5 and Table 3) encode a wide range of molecules essential for inflammation and colitis. For example, among the most highly expressed genes was Itgae (CD103) which has been shown to enable pathogenic T cells to migrate to their target tissue and is characteristic of pro-inflammatory colonic T cells in ulcerative colitis^{33,34}. Another highly expressed gene, Ccr9, encodes a gut homing receptor and has been shown to play a crucial role in colitis³⁵. Previously, it was shown that the transmembrane glycoprotein CD38 is important for intestinal inflammation³⁶. Likewise, CD48, a SLAM (signaling lymphocyte activation molecule) family member, is crucial for T cell function in colitis and it has been suggested that CD48 may represent a useful target in human IBD³⁷. Strikingly, an ORF (open reading frame) within the gene AW112010 (encoding a long-noncoding RNA) which was among the top 20 differentially regulated genes within cluster 9 in the data set has been recently shown to be essential in mucosal immunity including colitis³⁸. The expression of this ORF is fairly broad across clusters and the locus appears to be transcribed in the vast majority of cells. However, the expression of AW112010 shows a dramatic increase in expression within the inflammatory cluster 9 (FIG. 3d).

Importantly, in addition to the genes in cluster 9 that have been implicated in the development of colitis, the cluster also contained novel genes not previously associated with the development of colitis. For example, Cd160, the most upregulated gene in cluster 9 encodes an Ig superfamily member³⁹ and has not been assigned a function in colitogenic T cells yet. The gene encoding the nectin receptor family member Cd226 (DNAM-1), was also part of the signature of highly expressed genes in cluster 9. Co-stimulatory receptors such as CD226 play a critical role in T cell activation, autoimmunity and cancer⁴⁰. Previously, CD226 was shown to be crucial for the activation of cytotoxic lymphocytes and Th1 cells and its expression within cluster 9 suggests that it may play an important role in intestinal inflammation^{41,42}. In summary, the transcriptional analysis of cluster 9 exhibits an entire array of genes critical for intestinal inflammation and identifies novel potential genes important for intestinal inflammation such as Cd160.

Many genes highly expressed in cluster 2 are implicated in IFN-γ signaling (FIG. 3d and Table 3). In fact, IFN-γ signaling has early been identified as being critical for intestinal inflammation in various pre-clinical models of colitis^{31,43}. For example, top differentially genes in this cluster were Ifitm1 (interferon-inducible transmembrane protein), Ifitm2 and Ifitm3. Members of this family are interferon response genes that have been shown to be entry sites in viral infection⁴⁴. A study in human IBD patients identified IFITM1 as a potential prognostic marker in ulcerative colitis⁴⁵. Interestingly, Applicants identified many integrins as being part of the list of genes highly expressed within cluster 2, with Itgb1 as the second highest differentially expressed gene and which has previously been implicated in colorectal cancer⁴⁶. Two additional integrins highly upregulated in cluster 2 (albeit not among the top 20 differentially expressed genes) were Itga4 (position 101) and Itgb7 (position 32). Strikingly, a recent study demonstrated that targeting the leukocyte integrin a4p₇ with vedolizumab was more effective in moderately to severely active ulcerative colitis than adalimumab (a humanized monoclonal antibody neutralizing TNF)⁴⁷.

The observation, that many genes implicated in IFN-γ signaling and lymphocyte trafficking (integrins) were highly upregulated in cluster 2 suggests that this cluster consisted of inflammatory cells that could potentially represent the cellular phenotype of early potential drivers of the intestinal inflammatory response.

The other two clusters that were enriched in wildtype cells (Il23r^{eGFP/wt}) were clusters 8 and 10.

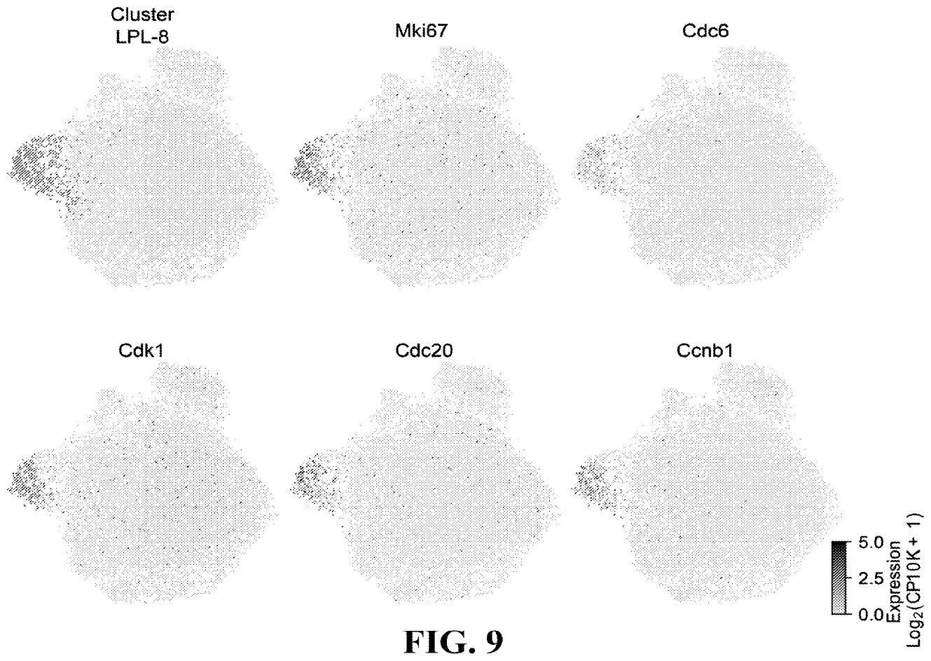

Cluster 8 represented a cluster of highly proliferating cells exemplified by the expression of Ki-67 and genes important for cell cycle progression such as Cdc (cell division cycle) genes, including Cdc6 (critical for the initiation of DNA synthesis⁴⁸) and Cdc20, which is known to activate the anaphase promoting complex/cyclosome APC/C⁴⁹ (FIG. 9). The observation that cluster 8 was highly enriched with wildtype cells suggests that these cells were actively proliferating within the tissue to contribute to the inflammatory process.

Cluster 10 on the other hand showed high expression of Ccr7 which is commonly correlated with an ability of T cell trafficking and homing to lymph nodes and Peyer's patches⁵⁰. In addition, cluster 10 cells expressed higher levels of the anti-apoptotic gene bcl2 which is known to be driven by STAT5 signaling and may be an interesting target in IBD as suggested in pre-clinical studies⁵¹.

In summary, using single-cell RNA-sequencing, Applicants identify distinct clusters of T cells within the intestinal mucosa that exhibit a highly inflammatory profile, in particular clusters 2 and 9. Furthermore, the observation that these clusters are dominated by wildtype cells suggests that IL-23R signaling critically contributes to the observed expression of inflammatory genes. Importantly, Applicants succeed in identifying novel genes such as Cd160 that may play an important role for intestinal inflammation and IBD.

### Example 7—IL-23R is Implicated in the Reciprocal Regulation of Tr-1 Like Cells and Limits their Expansion

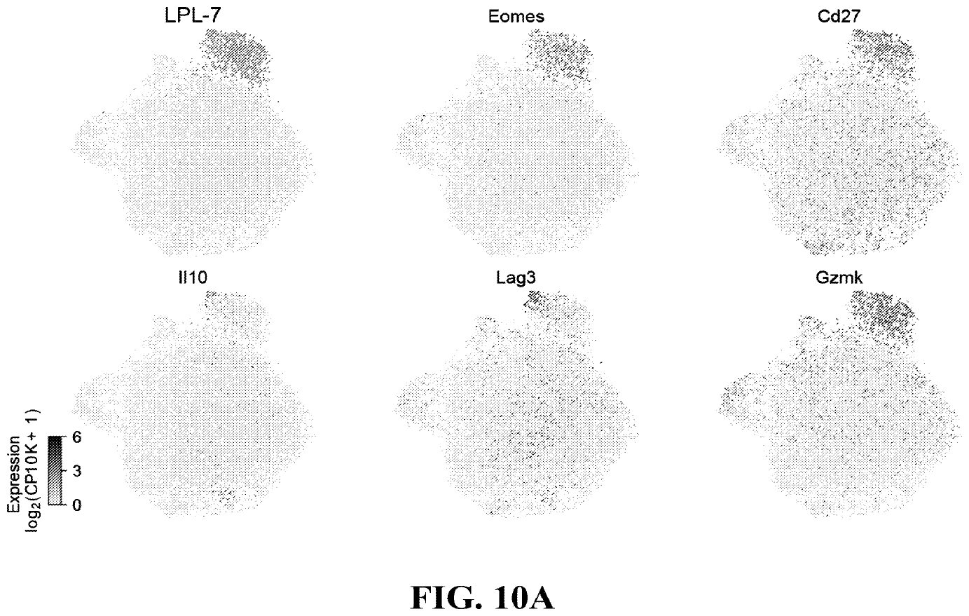

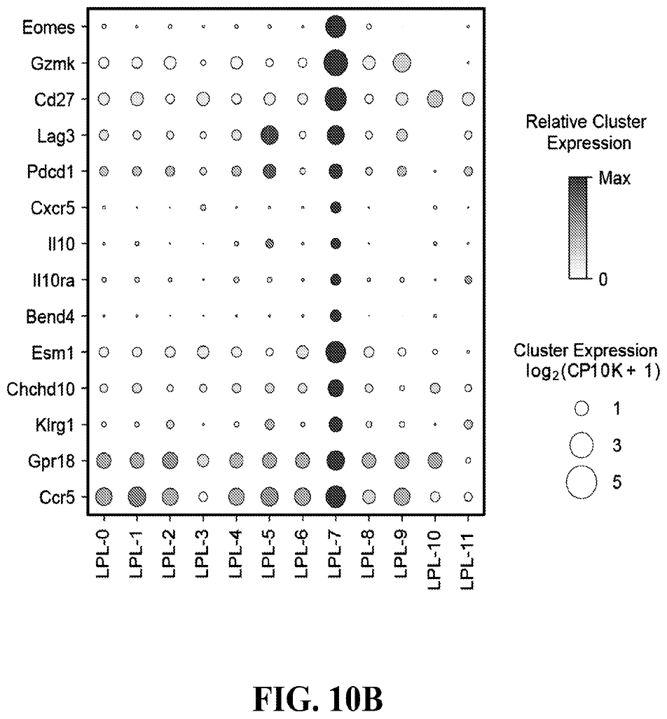

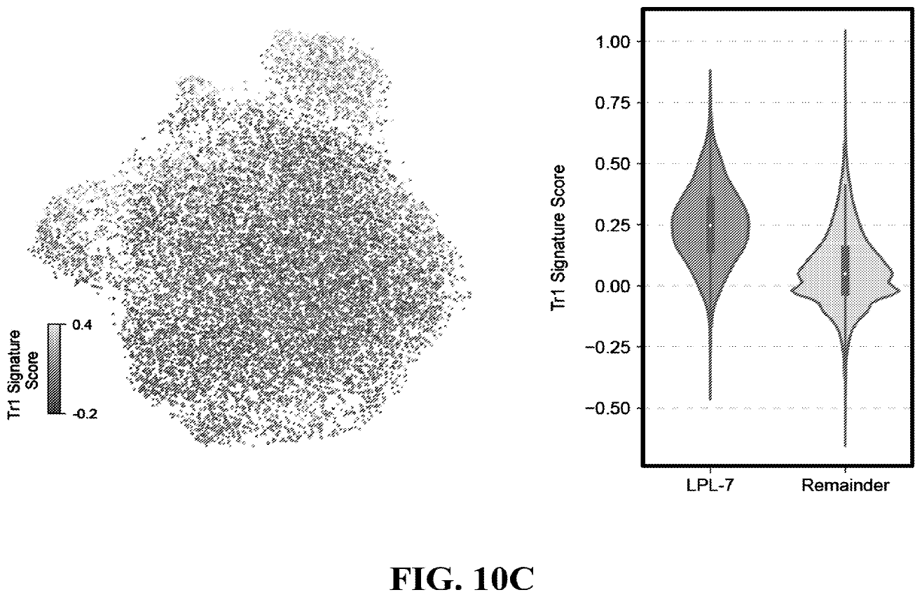

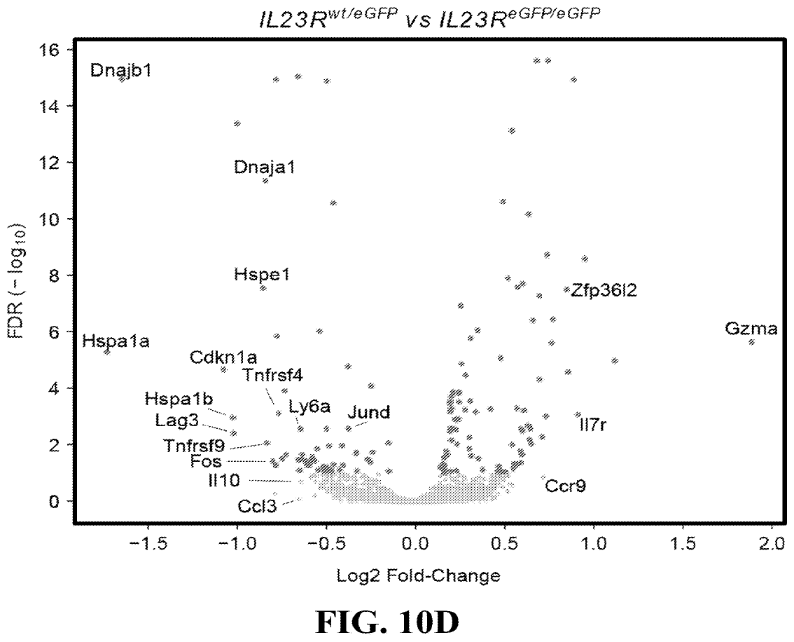

Cluster 7 was particularly interesting as it is strongly enriched with IL-23R KO cells which would suggest that this cluster contains predominantly non-pathogenic T cells (FIG. 3c). The 117 genes that characterize this cluster (one vs. all comparison, FDR<0.1, log FC>0.5) include Eomes, Cd27, Gzmk, Lag3 and Il10 (FIG. 3d and FIGS. 10a and b). This finding was of particular significance as these genes have been recently identified to define a novel human IFN-γ⁺IL-1β+Tr1-like cell type that may be reduced in IBD patients⁵². In fact, Eomesodermin (Eomes) has been shown to be a key transcription factor of a Tr1-like lineage⁵¹. The finding that cluster 7 is predominantly composed of IL-23R KO cells suggests that IL-23R may function as an important gatekeeper and negative regulator for the development of Tr1-like cells in the colonic mucosa. Previously, it was shown that IL-23R is indeed expressed on these IFN-γ⁺IL-1β+Tn-like cells and therefore IL-23 signaling could potentially regulate development and function of this population⁵⁴. To Applicants knowledge, this study is the first to provide functional evidence that IL-23R may act as a negative regulator of such Tr1-like cells. In addition, PD1 was shown to be expressed on human Tn-like cells which is highly consistent with the data presented here⁵⁴ (FIG. 3d). In fact, it was shown that PD1 is implicated in the conversion of human T-BET⁺ Th1 cells to FOXP3⁺ Tregs⁵⁵. Other co-inhibitory molecules such as Lag-3 were also more highly expressed in cluster 7 (FIG. 3d and FIG. 10b). Applicants used a published transcriptional signature that characterizes human Tr-1 like cells and were able to show that it identifies cluster 7 (upregulated compared to other clusters, Wilcoxon rank-sum test p<10⁻²⁰⁰) providing strong evidence that the cells of cluster 7 are the equivalent of these human cells in this study (FIG. 10c)⁵². In summary, the results suggest that IL-23R signaling balances the differentiation of colitogenic Th1 vs. Tr cells and therefore effector vs regulatory arms of the immune response.

Interestingly, comparing WT to KO cells within cluster 7 revealed a set of heat shock protein family members, in particular Hspa1a and Hspa1b (FIG. 10d). Heat shock proteins are implicated in the regulation of T cell responses and specific single nucleotide polymorphisms have recently been identified in HSPA1L that distinguish IBD patients from healthy controls^{56,57}. The finding suggests that heat shock proteins of the Hsp70 family, in particular Hspa1a and Hspa1b, may be protective in IBD and regulated in an IL-23R dependent manner.

### Example 8—Comparative Analysis of Human IBD GWAS with the scRNAseq Study Nominates Genes of Particular Interest for the Function of Colitogenic Th1 Cells in an IL-23R Dependent Manner

It has become clear from genome-wide association studies (GWAS) that IL-23R plays an important role in IBD⁵. To investigate if IL-23R signaling is particularly relevant to the expression of other genes found within human IBD GWAS loci in T cells, Applicants conducted a survey of the roughly 240 IBD risk loci that have been identified so far⁵⁸⁻⁶¹ and tallied their associated genes (about 700 genes in total)⁵⁸. Within loci that contain multiple genes, Applicants focused on genes that were found to be implicated, for example, by fine-mapping or by expressed quantitative trait loci (eQTL) analysis which reduced the list of genes that Applicants further investigated to 597. Since Applicants established that clusters 2 and 9 were dominated by wildtype cells with a highly inflammatory transcriptional signature Applicants focused the analysis on these two clusters in addition to cluster 7 which was dominated by KO cells to investigate the potential contribution of genes found in GWAS loci to IL-23R mediated pathogenicity. Applicants used within-cluster comparison of WT vs. KO cells and identified genes found within IBD GWAS loci that were expressed in an IL-23R dependent manner (FIG. 3e and Table 3). This finding suggests that IL-23R signaling in Th1 cells may be relevant to induction of genes implicated through GWAS in the development of IBD.

Among the genes in the set implicated through GWAS were Gpr18, Traf3ip2, Ncf4 and Ets1.

Gpr18 was particularly highly expressed in clusters 2 and 9 in an IL-23R dependent manner. Recently, it was shown that certain mutations are increased in ulcerative colitis including in the gene Traf3ip2 (encoding Act1) which is part of the NFKBIZ pathway⁶². Ncf4 plays an important role in cellular reactive oxygen species (ROS) pathways as part of the NOX2 (NADPH oxidase 2) complex. Ets1 is a transcription factor and essential co-factor of Tbet in Th1 cell-mediated inflammatory responses⁶³

Adhesion and migration regulate major biological outcomes of T cell-mediated autoimmunity. Interestingly, a number of genes implicated in these processes were part of the identified gene signatures of cluster 2, in particular Lsp1, Lpxn (Leupaxin) and Fermt3. Applicants hypothesize that IL-23R signaling may exert pathogenicity also through downstream effects on migration and adhesion.

Notably, some genes were not limited to clusters 2 and 9 in their IL-23R-dependent expression such as Zfp36l2 but exhibited this pattern also across other clusters including cluster 7 suggesting that their genotype-dependent expression may be too dominant than to be confined to a particular cluster (FIG. 3e). Zfp36l2 belongs to a family of RNA binding proteins that have been implicated in Th17 cell biology but their contribution to Th1 cell-mediated autoimmunity remains unknown^{11,64}.

The findings illuminate genes implicated in cellular pathways ranging from trafficking, cellular adhesion (integrins), metabolic regulators and immune cell interaction identified in GWAS IBD risk loci that may prove critical in their contribution to intestinal inflammation driven by IL-23R in colitogenic T cells. The observation that these genes found in GWAS IBD risk loci are driven by IL-23R in colitogenic Th1 cells may suggest that IL-23R and these genes converge mechanistically in a disease relevant manner.

### Example 9—Ranking Identifies Novel Genes as Drivers of Intestinal Inflammation in an IL-23R Dependent Manner

In many cases, the combinatorial analysis of several data sets yields a comprehensive insight into useful targets for further analysis as critical genes may not only emerge as being the top differentially expressed genes in a singular data set. Therefore, Applicants combined both in vitro and in vivo single-cell RNA-sequencing data sets Applicants reported above and included data from human IBD GWAS to rank the genes that may have an impact on the development of colitis.

Applicants postulated the following criteria that were important and queried the data set for genes that would match as many criteria as possible: (1) As Applicants were particularly interested in genes potentially conferring pathogenicity, Applicants focused on genes that were most highly expressed in clusters 2 and 9 in comparison to all other clusters and that were differentially expressed between wildtype and KO cells within either cluster 2 or 9; (2) Genes of particular importance to intestinal inflammation may exhibit a highly tissue specific expression and therefore Applicants evaluated their expression comparing splenic and intestinal samples; (3) Applicants asked which genes showed an TL-23R dependent expression in Th1 cells differentiated under IL-12+IL21+TL-23 in vitro; (4) Applicants incorporated as one criterion whether a given gene was previously identified in human GWAS to confer risk and/or is located within a GWAS risk locus for IBD (FIG. 4a and Table 4, see methods for details of ranking).

Gpr18 scored very highly in all criteria including being located within a GWAS risk locus⁶¹. Interestingly, it was shown that Gpr18 plays a crucial role for CD8aa⁺ intraepithelial T lymphocytes within the intestinal mucosa⁶⁵. The studies suggest it may be a critical mediator of pathogenicity by IL-23R in colitogenic Th1 cells.

The transcription factor NF-fB is critical to T cell function and in particular mutations within the NFKBIZ pathway were recently found to be relevant in ulcerative colitis⁶². Nfkbiz itself scored highly in the ranking data as a target in colitogenic T cells (FIG. 4a).

### Example 10—CD160 Plays an Important Role in Colitogenic Th1 Cells

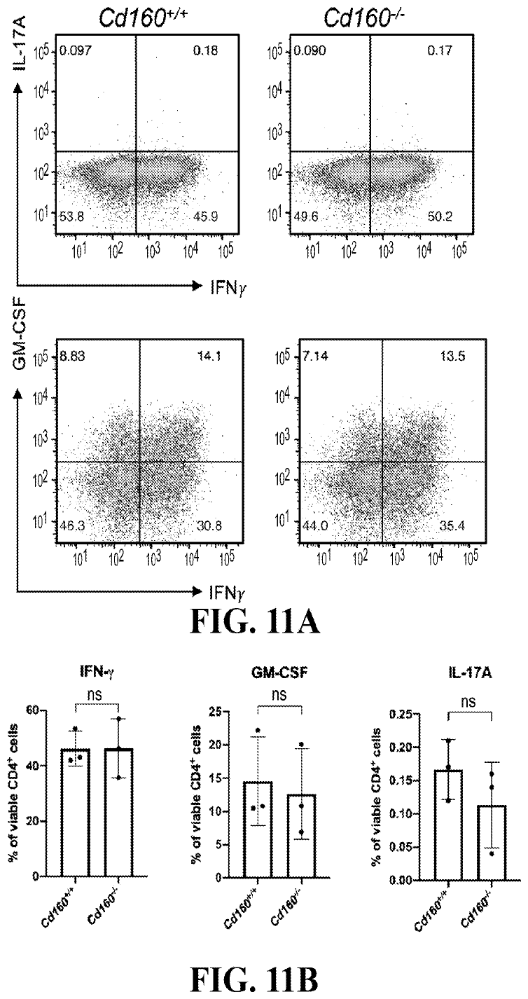

Finally, Applicants sought to provide conclusive evidence of the critical contribution of candidate genes identified in the studies to T cell-driven intestinal inflammation. To this end, Applicants acquired knockout mice for Cd160, the top differentially expressed signature gene of inflammatory cluster 9 and which ranked prominently in the ranking scheme (FIG. 4a)⁶⁶. CD160 is an Ig superfamily member and its function in inducing colitogenic T cells and IBD has not been investigated. It has been shown to be important for IFN-γ production in NK cells and being expressed on intraepithelial CD8⁺ T cells^{66,67}. Applicants differentiated naïve T cells from these CD160 KO mice to Th1 cells in vitro as described above and tested their ability to elicit adoptive transfer colitis. Applicants did not observe a difference between wildtype and CD160 KO Th1 cells to produce IFN-γ or GM-CSF prior to adoptive transfer in vitro (FIGS. 11a and b). The cells did not express any IL-17A prior to adoptive transfer. Strikingly, the recipient animals that received CD160 deficient Th1 cells showed a strong protection from colonic inflammation as assessed by histopathological examination (FIG. 4b). The decrease of inflammation showed a clear trend in the small intestine and was statistically significant in the colon (FIGS. 4b and c) The histopathological analysis was consistent with the shortening of the colon in recipients of wildtype cells (FIG. 4d). When Applicants isolated T cells from mesenteric lymph nodes from recipients of either wildtype or CD160 knockout cells Applicants did not observe a significant difference in IFN-γ expression which was consistent with the observations in vitro suggesting that it was likely not any lack in the ability to produce IFN-γ that protected recipients of CD160 KO cells from developing severe colitis (data not shown). Previously, it was shown that CD160 is important for Gzmb expression in intestinal CD8+ T cells which helps to clear Listeria monocytogenes infection⁶⁶. Interestingly, this was highly consistent with the observation that CD160 KO cells differentiated in vitro with IL-12+IL+21+IL23 produced less Gzmb compared to wildtype cells which could contribute mechanistically to their diminished colitogenicity in vivo (FIG. 4e). Taken together, the results demonstrate an important function of CD160 to the colitogenicity of Th1 cells in an IL-23R dependent manner.

### Example 11—Discussion

IL-23R function has been considered a critical driver of pathogenicity in Th17 cells and important for the regulation of FoxP3⁺ Tregs but not for other T helper cell subsets. However, several experimental and clinical observations have been difficult to reconcile under this assumption. Applicants provide compelling evidence that IL-23R plays an important role in other pathogenic T cell subsets, in particular a subset of IL-23R⁺ Th1 cells.

First, Applicants report that bonafide Th1 cells can be differentiated in vitro with IL-12+IL-21 and express similar levels of IL-23R as pathogenic Th17 cells polarized with IL-1β+IL-6+IL-23. This illustrates how the combinatorial action of cytokines crucially determines the functional outcome since IL-21 has been shown to participate with TGF-β to induce pro-inflammatory Th17 cells, not Th1 cells⁶⁸. In Th17 cells, IL-6 has been shown to induce IL-21 that promoted IL-23R expression in an autocrine manner, and this induction was STAT3 dependent⁶⁹. Which transcription factor mediates the induction of IL-23R in Th1 cells differentiated with IL-12+IL-21 remains to be determined. However, one such transcription factor could be c-MAF that has been shown to bind to the IL-23R promoter and to work in conjunction with IL-21 in some non-Th17 cells, in particular in the setting of autoimmune diabetes which is known to be Th1 cell-driven⁷⁰⁻⁷².

Applicants continue to show that IL-23R is required in Th1 cells in vivo to acquire full pathogenicity to elicit adoptive transfer colitis. Applicants take advantage of scRNAseq to profile 32,763 cells in a pre-clinical model of colitis including roughly 20000 intestinal T cells and uncover transcriptional signatures that are governed by IL-23R and that are critical to the emergence of pathogenic and colitogenic Th1 cells. The inflammatory signatures of the pre-clinical model were highly reminiscent of human studies in IBD as outlined above. Applicants identify novel candidate genes for T cell-mediated IL-23R dependent pathology and provide a framework of genes identified in previous IBD GWAS as potential drivers in colitogenic Th1 cells. The study therefore contributes to the important effort of identifying and validating genes found within GWAS risk loci having an important function in colonic inflammation and their implication in colitogenic T cells.

Previously, it was shown that Th17 cells show extensive plasticity during an inflammatory response and that Th17 cells eventually may develop features of Th1 cells by upregulating Tbx21 and expressing IFN-γ both in mouse and human¹⁹⁷³,7⁴. IL-23R was shown to play an important role in this lineage plasticity¹⁹ and it was assumed that IL-23R bearing Th cells that express IFN-γ are solely transdifferentiated Th17 cells. Interestingly, it was shown in the adoptive transfer model of colitis that Th17 cells convert to Th1 cells and that this appears to be essential to Th17 cell mediated disease in this model⁷. Importantly, Applicants show that bonafide Th1 cells can be differentiated to express Il23r in vitro and that IL-23R deficient Th1 cells lose their ability to elicit intestinal inflammation. Interestingly, Applicants did not observe extended plasticity in the Th1 cells adoptively transferred in the study towards a Th17 cell phenotype. With the exception of cluster 3 in the LPL that contained cells showing a transcriptional signature of Th17 cells including expression of Ccr6, Il17a, Il22, Il17re and Il17f, however, Applicants did not identify IL-17A⁺ cells by intracellular cytokine when retrieved from the intestinal mucosa in RAG1 KO recipients. Thus, the vast majority of LPL cells showed a strong transcriptional signature of bona fide Th1 cells including expression of Il12rb1, Il12rb2, Cxcr3, Stat1, Stat4, Tbx21 and Ifng.

Applicants speculate that Th1 cells represent a further advanced state of inflammatory trajectory that may be reached with or without transitioning through a Th17 cell state. Strikingly, it has been demonstrated that both Th17 and Th1 cells may ultimately give rise to T cells with regulatory function at the resolution of inflammation^{55,75}.

Importantly, Applicants describe here a dichotomous function of IL-23R in promoting inflammatory cells on one hand and on the other hand limiting the expansion of Tr cells with a regulatory function, identified by expression of signature genes such as Eomes, Cd27, Il10ra, Il10, Gmzk and co-inhibitory receptors Lag-3 and Pdcd1. A concept is evolving in which IL-23R serves not only as a critical driver of inflammatory T cells but a central integrator of immune responses within the local immunological milieu. In fact, in addition to the emergence of Tr1-like cells in IL-23R deficient cells in the study, it has been shown previously that IL-23R is also expressed on colonic FoxP3⁺ Tregs, making these cells receptive to sudden inflammatory changes that limit their suppressive effector functions in the presence of the pro-inflammatory cytokine IL-23^{14,15}.

In the setting of Th17 cells, it was shown that the salt-sensing kinase Sgkl is important in regulating their plasticity toward FoxP3⁺ T cells⁷⁶. Which mediators downstream of IL-23R signaling suppress a Tr1 cell fate in Th1 cells remains to be determined.

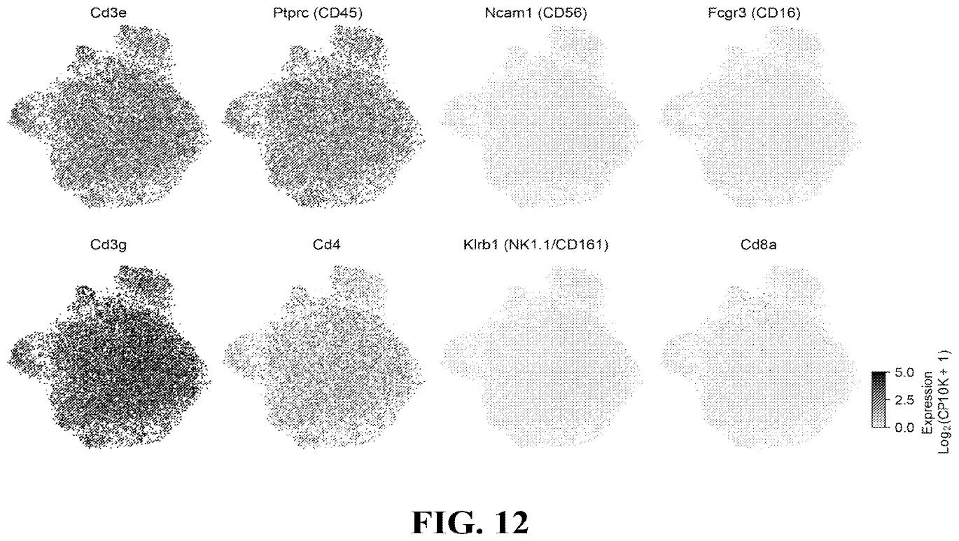

A ranking scheme combining both in vitro and in vivo transcriptional signatures and their dependence on IL-23R signaling enabled us to identify novel drivers of T cell pathogenicity and Applicants succeeded in validating one of these candidates, CD160. Indeed, CD160 emerged in a recent study profiling the cellular composition and transcriptional signatures of ileal biopsies from Crohn's patients although the functional significance remained unclear⁷⁷. Interestingly, CD160 was shown to be very important for intraepithelial type I ILCs which are amplified in Crohn's disease and was important for the pathogenicity of ILCis in the anti-CD40 colitis model in addition to control of L. monocytogenes infection. These data suggest that CD160 is not only important for CD4⁺ T cells as demonstrated in the study but for other immune cells such as ILC1s and CD8⁺ T cells as well^{66,78}. To validate that Applicants had not inadvertently included immune cells other than CD3⁺CD4⁺ T cells in the analysis that have been shown to express CD160, Applicants confirmed that the cells sequenced lacked expression of NK cell markers and expression of CD8 (FIG. 12).

Another important candidate that emerged and which scored highly in all categories in the ranking (FIG. 4a) was Gpr18. Interestingly it was identified as a central part of a transcriptional signature in myeloid cells driving a Th1 cell intestinal inflammation triggered by the colonization of germ-free mice with Klebsilla isolated from a Crohn's patient⁷⁹

In summary, Applicants uncouple IL-23R as a purely Th17 cell-specific pathogenicity factor and implicate it in being critical for evoking a pathogenic phenotype in Th1 cells. Furthermore, through the powerful combination of a highly relevant pre-clinical model of colitis with massively parallel single-cell RNA-sequencing and integration of GWAS data from IBD patients, Applicants identify novel potential drivers of IL-23R-mediated T cell pathogenicity and validate one of these candidates. Applicants hope that the discovery of the role of IL-23 in the induction of pathogenic Th1 cells and their contribution to autoimmune disease on one hand and the inhibition of IL-10 producing Tr cells on the other hand may identify novel targets and prove useful for the development of therapeutic intervention in multiple human autoimmune diseases.

### Example 12—Materials and Methods

Mice. IL-23R^{eGFP} reporter mice were described previously (Awasthi et al., 2009). The following strain was purchased from The Jackson Laboratories: B6.129S7-Rag1^{tm1Mom}/J (known as Rag1^{-/-}; Stock No.: 002216). CD160^{-/-} mice were a kind gift of Dr. Arlene Sharpe and previously described (Tan et al., 2018). All animals were bred and maintained under specific-pathogen-free (SPF) conditions. Genotyping was performed with DNA isolated from tail biopsies. All experiments were approved by and carried out in accordance with guidelines of the Institutional Animal Care and Use Committee (IACUC) at Harvard Medical School and Brigham and Women's Hospital.

In vitro T cell differentiation. A single-cell suspension was prepared from spleen and lymph nodes and red blood cells were removed by lysis with ACK buffer (Lonza). CD4⁺ T cells were enriched using microbeads (Miltenyi Biotec, Cat. #.: 130-117-043). Naïve CD4⁺ T cells (CD4⁺CD44^{low} CD62L^{high}CD25⁻) were isolated by fluorescence-activated cell sorting (FACS) using a custom-built BD FACSAria instrument. Sorted naïve CD4+ T cells were activated with plate-bound anti-CD3 and anti-CD28 antibodies (1 μg/ml; BioXCell; Clone 145-2C11 and clone PV-1, respectively) in 96-well flat-bottom plates (50-100×10³ cells/per well). Cells were cultured for 5 days (cytokine screen) with the following cytokine concentrations: IL-1β (20 ng/ml); IL-6 (25 ng/ml); IL-23 (20 ng/ml); IL-12 (20 ng/ml); IL-21 (20 ng/ml); TGF-β (2 ng/ml); IL-18 (20 ng/ml). All recombinant cytokines were purchased from Biolegend (IL-21), R&D (IL-12, IL-1β, IL-6, IL-23) and Miltenyi Biotec (hTGF-β).

Flow cytometry and antibodies. Single-cell suspensions were stained with diluted antibodies (1:100) in ice-cold PBS+2% FBS. Dead cells were identified with either 7-AAD (BD Biosciences) or viability dye eFluor 506 (eBioscience). For intracellular cytokine staining (ICC), cells were stimulated for 4 hr at 37° C. with phorbol 12-myristate 13-acetate (PMA, Sigma), ionomycin (Sigma) and monensin (GolgiStop; 1:1000; BD Biosciences). Then, cells were stained for surface markers and viability. Cells were fixed and permabilized according to the manufacturer's instructions (BD Cytofix/Cytoperm kit). All flow cytometric data were acquired with either a BD LSRII or Fortessa instrument and analyzed with FlowJo (BD).

The following antibodies labeled with different fluorophores were purchased from Biolegend unless otherwise indicated: anti-CD4 (RM4-5), anti-CD25 (PC61), anti-CD44 (IM7), anti-CD62L (Mel14), anti-IL-17A (TC11-18H10.1), anti-IFN-7 (XMG1.2), anti-GM-CSF (MP1-22E9, BD), anti-CD45 (30-F11).

Adoptive transfer colitis. Naïve T cells were isolated from donor mice by FACS and differentiated for 6 days prior to adoptive transfer with the following paradigm. Naïve CD4⁺ T cells (in 6 well dishes, 1×10⁶ cells/ml) were cultured for 48 hrs on anti-CD3/anti-CD28 coated plates with the cytokines IL-12 (10 ng/ml)+IL-21 (20 ng/ml). After 48 hrs, the cells were passaged onto non-coated plates and IL-2 (20 ng/ml) was added to the culture. Cells were passaged if needed 1:2. On day 4, IL-23 (20 ng/ml) was added to the culture. During the entire culture, the medium was supplemented with anti-IL-4 antibody (BioXcell; clone 11B11). On day 5, a small sample was taken and used for ICC to determine IFN-γ⁺ cells and cells to be adoptively transferred were re-stimulated by passage onto anti-CD3/anti-CD28-coated plates for at least 24 hrs. After 6 days, cells were harvested, washed in ice-cold PBS and viability was assessed with Typan Blue staining. Cells to be injected were normalized based on IFN-γ expression determined by ICC and 400000 viable IFN-γ⁺ cells were injected intraperitoneally per animal. As recipients, sex-matched RAG1^{-/-} animals were used. The weight was measured pre-adoptive transfer and routinely over the course of the experiment.

For histopathology, the colon and small intestine were fixed in 10% formalin and processed by the Harvard Medical School rodent histopathology core for H&E staining. The severity of disease was assessed in a blinded manner by an experienced histopathologist. The following scoring system was used: 0 (healthy)-4 (most severe disease).

Isolation of intestinal lymphocytes. Intestinal lymphocytes were isolated from RAG1^{-/-} recipients using a lamina propria dissociation kit according to the manufacturer's instructions (Miltenyi Biotec, Cat. No.: 130-097-410). Briefly, intestines were isolated and flushed with ice-cold buffer. The small intestine and colon were separated, opened longitudinally with scissors, turned inside-out and then cut into small pieces (<1 cm). Then, the intraepithelial lymphocytes (IEL) were shaken off with buffer containing 1 mM DTT with gentle rotation at 37° C. After the incubation period, the IEL were swiftly filtered (100 μm), washed with complete medium and kept on ice. This step was repeated once followed by a washing step to remove DTT. Then, the procedure was continued to isolate the lamina propria lymphocytes (LPL). For digestion of the intestinal mucosa, the samples were gently incubated with a proprietary enzymatic solution in C-tubes (Miltenyi Biotec, Cat. No.: 130-096-334) at 37° C. for at least 30 minutes using a rotator. After the enzymatic incubation, the samples were homogenized using a gentleMACS (Miltenyi Biotec) and the program m_intestine_01.

For overnight culture, to assess the cytokine profile of intestinal T cells, the cells were further isolated using a Percoll (GE Life Sciences/Cytiva) gradient centrifugation to remove cell debris and fat tissue.

Analysis of ex vivo cytokine production by intestinal lymphocytes. IEL and LPL were cultured overnight in complete medium with IL-7 (20 ng/ml, Biolegend)+IL-23 (20 ng/ml, R&D). The next day, the cells were stimulated with PMA/ionomycin+GolgiStop for 4 hrs as described above. ICC was performed and the cells stained for IL-17A, IFN-γ and GM-CSF.

Single-cell RNA-sequencing (scRNAseq). For full-length scRNAseq, single, viable in vitro differentiated CD4+ T cells were sorted into 96-well plates containing 5 μl TCL Buffer (QIAGEN) with 1% 2-mercaptoethanol, centrifuged and frozen at −80° C. SMART-Seq2 protocol was carried out mainly as previously described (Singer et al., 2016). For droplet-based 3′ end massively parallel single-cell RNA-sequencing, sorted (FACS) CD45+CD4+ intestinal T cells were encapsulated into droplets, and libraries were prepared using Chromium Single Cell 3′ Reagent Kits v2 according to the manufacturer's protocol (10×Genomics). Generated libraries were sequenced on a HiSeq X (Illumina).

RNA isolation and quantitative PCR. Total RNA was isolated with the RNeasy kit according to the manufacturer's instructions (Qiagen). In brief, cells were homogenized with 200μl RLT buffer supplemented with 2-mercaptoethanol by gentle pipetting and then an equal amount of 70% ethanol was added, the samples were mixed by inversion and RNA was captured through a centrifugation step with silica-based columns. Purified RNA was reverse-transcribed using Superscript II enzyme and random hexamer primers (Invitrogen). Taqman probes for genes of interest were purchased from Applied Biosystems. A ViiA7 Real-time PCR system (Applied Biosystems) was used for amplification and data acquisition.

Taqman probes

| Taqman assay | Gene |
|---|---|
| Mm00519943_m1 | Il23r |
| Mm01168134_m1 | Ifng |
| Mm00439618_m1 | Il17a |
| Mm00450960_m1 | Tbx21 |
| Mm01261022_m1 | Rorc |
| Mm01219775_m1 | Stat3 |

Statistical analysis. Details regarding the statistical analyses can be found in the figure legends. Statistical analysis was performed using GraphPad Prism 8.0. A p-value p<0.05 was considered significant.

Analysis of in-vitro SmartSeq2 single-cell transcriptional profiles. Sequencing reads were initially filtered with Trimmomatic 0.32⁸⁰ to remove low quality ends and adapter sequences. A combination of Bowtie2 v2.2.9⁸¹ was used to align reads to a reference transcriptome derived from the GRCm38 Mus musculus genome (NCBI Refseq). RSEM 1.2.31⁸² was used to compile the transcriptome and estimate transcript abundance following read alignment. Prior to alignment, the GRCm38 reference was augmented to include the modified eGFP+IL23R functional knockout transcript.

Prior to downstream analysis, cell libraries were excluded if they contained less than 800,000 reads or more than 3 million reads. Additionally, libraries with less than a 40% alignment rate were withheld. Finally, genes detected in less than 5 cell libraries were discarded prior to differential expression.

Initial inspection of the RNA-seq libraries showed that the dominant source of variation between cells could be attributed to differences in cell cycle progression. To reduce the effect of cell cycle on differential expression testing, two cell-cycle ‘scores’ per cell were computed and input as covariates into the differential expression model. To compute these scores, Applicants first subset the expression matrix, retaining only 97 genes identified as G1/S or G2/M enriched genes in a previous study⁸³. Then, PCA was run on the log(TPM+1) expression values and the first two component scores per cell were extracted.

To identify differentially expressed genes, the MAST⁸⁴ R package was used. When fitting the hurdle model, in addition to the covariates of interest both cell cycle components (described above) as well as the number of detected genes per cell were used to reduce false positives due to cycling state or mRNA capture rates. P-values were corrected with the Benjamini Hochberg FDR procedure and genes were accepted at an FDR<0.1 threshold. To identify genes whose expression differed between the GFP⁺ and GFP⁻ subsets in a genotype-dependent manner (and therefore may be perturbed as a consequence of Il-23 receptor signaling), data from all four subsets (Il23R^{WT/eGFP} and Il23R^{eGFP/eGFP}, GFP^{+/-} both) were combined and modeled with categorical covariates for GFP, Genotype, and a GFP:Genotype interaction term (in addition to the cell cycle and detection rate terms). Genes whose expression differed between the FACS sorted GFP subsets were first identified by comparing this full model to a reduced model without the GFP and GFP:Genotype terms, retaining those with FDR<0.1. Then, in this subset, Applicants tested the GFP:Genotype interaction term specifically and retained genes with FDR<0.1.

For visualization, the dataset was reduced using principal components analysis (PCA) followed by tSNE⁸⁵. Prior to running PCA, the log(x+1) transformed TPM expression values were normalized by regressing out three cell-level covariates: the number of detected genes, and both cell-cycle components. The top 30 components from PCA were then used as input to the tSNE procedure (with perplexity=30) to further reduce the data to 2 dimensions.

When comparing the GFP-associated expression between T cells stimulated under Th1 or Th17-producing conditions, an effect was considered “common” using the criteria FDR<0.05 in at least one comparison and (unadjusted) p-value <0.05 in the other with a log fold-change in the same direction. Opposite effects satisfied the same significance criteria but with a log fold-change of opposing signs, and Th1/Th17-specific effects satisfied FDR<0.05 in one group but p-value >0.05 in the other.

Analysis of In-Vivo 10×-Sequenced Single-Cell Libraries

Alignment and Preprocessing. To quantify gene expression, samples were individually aligned to the mm10 Mus musculus genome using CellRanger v2.0.2. In total, 14 samples were collected, however two samples of intraepithelial lymphocytes in the small intestine from batch 1 were discarded as the IL23R knockout sample did not pass quality checks (insufficient barcodes belonging to cells). The remaining 12 samples were used in downstream analysis.

Merging and Filtering. An initial analysis was performed on the concatenation of all samples to identify and remove dying or contaminating cell types. All 12 samples were modeled with scVI⁸⁶, selecting genes expressed in at least 100 cells, using 10 latent components, and encoding each sample ID as a batch variable so as to remove tissue and genotype specific effects.

The resulting latent space was then clustered into 11 clusters using the Louvain algorithm as implemented in SCANPY⁸⁷ with the following settings: n_neighbors=10 and resolution=0.5. Of these four clusters were marked for removal in all subsequent analysis (across all samples), in total consisting of 7.6% of all 32,763 cells.

One cluster, c8, was characterized by low recovered UMI counts, a low proportion of ribosomal coding RNA, a high proportion of mitochondrial RNA, and very few other distinguishing genes. These were removed as being likely low-quality measurements from damaged cells.

Another cluster, c9, was identified as a possible contaminant cell type due to marked lack of CD4 and CD52 expression. Similarly, cluster c10 lacked T cell markers and had high expression of hemoglobin-related genes.

Finally, cluster c6, despite exhibiting Th17-related genes (Ccr6, Il-23r, Rorc, and IL-22), was removed as the lack of CD3 expression, lack of TCR expression, and distinct lack of any IL-23r-eGFP detection (despite significant Il-23r detection), indicated that these were likely LTi ILC3 cells from the recipient mouse and therefore not part of the genotype-perturbed donor cells of interest in this experiment. After this stage of filtering, a total of 30,259 cells remained for downstream analysis.

Between Tissue Analyses. The combined UMAP plots of FIG. 2 were generated by first modeling all cells with scVI⁸⁶, and then running the UMAP⁸⁸ algorithm using SCANPY⁸⁷ with n_neighbors=30. When running scVI, the two experimental batches were encoded as the batch variable and zero-inflation in the model was disabled. Additionally, genes were pre-filtered to retain A) genes expressed in at least 100 cells and B) genes with high fano factor: genes divided into 30 bins based on mean, fano factor computed for each gene—using the UMI-depth normalized counts—and genes retained with a fano factor greater than 2 MAD (median absolute deviations above median) per bin.

For the heatmap in FIG. 2E, edgeR⁸⁹ was run to compute average expression per tissue and to determine the genes with the highest tissue specificity, the top 500 of which were plotted. The model was fit in the form “Batch+Tissue” and the Tissue coefficient was tested with the likelihood ratio test. When computing within-tissue differential expression based on Genotype, edgeR was used to compute differential expression (between control and eGFP/eGFP cells) using the likelihood ratio test.

Within-Tissue Analysis: Lamina Propria Lymphocytes. Samples from the lamina propria (6 total—batch 1: control and knockout in colon and batch: 2 control and knockout in colon and small intestine) were grouped together and modeled with scVI⁸⁶ (8 components, zero-inflation enabled, genes retained that were expressed in 10 cells or more and the Fano filter as described in the previous section). As broad genotype-specific differences in expression (e.g., heat shock proteins), prevented cells from clustering together, Applicants regressed out individual sample differences as if they were batch (i.e., encoded sample ID as the batch variable) and instead assessed the resulting clusters in terms of proportional representation (control vs. knockout) and within-cluster differential expression (control vs knockout). UMAP and Louvain clustering were run on the resulting 8 components as implemented in SCANPY⁸⁷ with the following parameters: n_neighbors=30 and (for Louvain) resolution=0.9.

Changes in cluster composition were adjusted by the number of cells recovered using the following relation:

$$Genotype Ratio = ( \frac{Control Cells in Cluster}{Total Control Cells} ) / ( \frac{Knockout Cells in Cluster}{Total Knockout Cells} )$$

To identify marker genes per cluster, edgeR⁸⁹ was used to compute log fold-change and significance when comparing cells in the cluster vs all other cells (1 vs. all differential expression). Similarly, edgeR was used to determine differential expression within cluster (control vs. knockout cells).

The Tr1 signature from Gruarin et al., 2019⁵² is composed of the 51 genes differentiated Tr1 cells from other CD4 cells in FIG. 1A of that study. Per-cell signature scores were calculated using VISION⁹⁰ and the expression matrix scaled to the median number of UMIs per cell.

Target Prioritization (Ranking)

For each gene, Applicants computed an overall score with four components. Genes were then ranked by a weighted sum of these components. The components are defined as:

1. In-vivo, Cluster Specific—the maximum of:
The log fold-change of genes significant (FDR<0.1) in the cluster 2 vs. all comparison
The log fold-change*0.5 of genes significant (FDR<0.1) in the cluster 9 vs. all comparison
The log fold-change of genes significant (FDR<0.1) in the within-cluster (cluster 2) control vs. knockout comparison
The log fold-change*0.5 of genes significant (FDR<0.1) in the within-cluster (cluster 9) control vs. knockout comparison
   2. In-vivo, Tissue Specific—the maximum of:
The log fold-change for genes significant (FDR<0.1) in the LPL vs. Spleen comparison
The log fold-change for genes significant (FDR<0.1) in the within-LPL control vs. knockout comparison

For both the above in-vivo categories, negative log fold-changes were truncated at 0 (to select specifically for genes upregulated in the cluster of interest or in control cells) and the overall score per gene was limited to 2.

3. In-vitro, Summary—the maximum of:
The log fold-change for genes with significant (FDR<0.1) genotype-independent differences in the in-vitro GFP+vs. GFP− comparison
The log fold-change of the genotype-dependent effect (GFP+×Genotype Ctrl) in the in-vitro linear model

For the above, in-vitro category, the overall score per gene was limited to 3.

### 4. GWAS:

Applicants assigned a GWAS score of 1 for genes within GWAS loci for Ulcerative Colitis or Crohn's Disease (as identified by Jostins et. al., 2012; Liu et. al., 2015; DeLange et. al., 2017^{58,60,61} and compiled by DeLange et al., 2017⁵⁸). Alternate genes were discarded for loci in which a specific gene has been confidently implicated by fine-mapping, eQTL, or target sequencing studies (as compiled by DeLange et al. 2017⁵⁸).

The final ranking was determined by combining all four categories using the following weightings:

Overall Score=(In-vivo,Cluster Specific)×2+(In-vivo,Tissue Specific)×1+(In-vitro,Summary)×1+(GWAS)×2

### REFERENCES

1 Cua, D. J. et al. Interleukin-23 rather than interleukin-12 is the critical cytokine for autoimmune inflammation of the brain. Nature 421, 744-748, doi:10.1038/nature01355 (2003).
2 Murphy, C. A. et al. Divergent pro- and antiinflammatory roles for IL-23 and IL-12 in joint autoimmune inflammation. J Exp Med 198, 1951-1957, doi:10.1084/jem.20030896 (2003).
3 Ahern, P. P. et al. Interleukin-23 drives intestinal inflammation through direct activity on T cells. Immunity 33, 279-288, doi:10.1016/j.immuni.2010.08.010 (2010).
4 Teng, M. W. et al. IL-12 and IL-23 cytokines: from discovery to targeted therapies for immune-mediated inflammatory diseases. Nat Med 21, 719-729, doi:10.1038/nm.3895 (2015).
5 Duerr, R. H. et al. A genome-wide association study identifies IL23R as an inflammatory bowel disease gene. Science 314, 1461-1463, doi:10.1126/science.1135245 (2006).
6 Hueber, W. et al. Secukinumab, a human anti-IL-17A monoclonal antibody, for moderate to severe Crohn's disease: unexpected results of a randomised, double-blind placebo-controlled trial. Gut 61, 1693-1700, doi:10.1136/gutjnl-2011-301668 (2012).
7 Harbour, S. N., Maynard, C. L., Zindl, C. L., Schoeb, T. R. & Weaver, C. T. Th17 cells give rise to Th1 cells that are required for the pathogenesis of colitis. Proc Natl Acad Sci USA 112, 7061-7066, doi:10.1073/pnas.1415675112 (2015).
8 Ghoreschi, K. et al. Generation of pathogenic T(H)17 cells in the absence of TGF-beta signalling. Nature 467, 967-971, doi:10.1038/nature09447 (2010).
9 Lee, Y. et al. Induction and molecular signature of pathogenic TH17 cells. Nature immunology 13, 991-999, doi:10.1038/ni.2416 (2012).
10 Yosef, N. et al. Dynamic regulatory network controlling TH17 cell differentiation. Nature 496, 461-468, doi:10.1038/nature11981 (2013).
11 Gaublomme, J. T. et al. Single-Cell Genomics Unveils Critical Regulators of Th17 Cell Pathogenicity. Cell 163, 1400-1412, doi:10.1016/j.cell.2015.11.009 (2015).
12 Patel, D. D. & Kuchroo, V. K. Th17 Cell Pathway in Human Immunity: Lessons from Genetics and Therapeutic Interventions. Immunity 43, 1040-1051, doi:10.1016/j.immuni.2015.12.003 (2015).
13 McGeachy, M. J. et al. The interleukin 23 receptor is essential for the terminal differentiation of interleukin 17-producing effector T helper cells in vivo. Nature immunology 10, 314-324, doi:10.1038/ni.1698 (2009).
14 Izcue, A. et al. Interleukin-23 restrains regulatory T cell activity to drive T cell-dependent colitis. Immunity 28, 559-570, doi:10.1016/j.immuni.2008.02.019 (2008).
15 Schiering, C. et al. The alarmin IL-33 promotes regulatory T-cell function in the intestine. Nature 513, 564-568, doi:10.1038/nature13577 (2014).
16 Sandborn, W. J. et al. Ustekinumab induction and maintenance therapy in refractory Crohn's disease. N Engl J Med 367, 1519-1528, doi:10.1056/NEJMoa1203572 (2012).
17 Sands, B. E. et al. Ustekinumab as Induction and Maintenance Therapy for Ulcerative Colitis. N Engl J Med 381, 1201-1214, doi:10.1056/NEJMoa1900750 (2019).
18 Powrie, F. et al. Inhibition of Th1 responses prevents inflammatory bowel disease in scid mice reconstituted with CD45RBhi CD4+ T cells. Immunity 1, 553-562 (1994).
19 Hirota, K. et al. Fate mapping of IL-17-producing T cells in inflammatory responses. Nat Immunol 12, 255-263, doi:10.1038/ni.1993 (2011).
20 Szabo, S. J. et al. A novel transcription factor, T-bet, directs Th1 lineage commitment. Cell 100, 655-669, doi:10.1016/s0092-8674(00)80702-3 (2000).
21 Mosmann, T. R., Cherwinski, H., Bond, M. W., Giedlin, M. A. & Coffman, R. L. Two types of murine helper T cell clone. I. Definition according to profiles of lymphokine activities and secreted proteins. J Immunol 136, 2348-2357 (1986).
22 Awasthi, A. et al. Cutting edge: IL-23 receptor gfp reporter mice reveal distinct populations of IL-17-producing cells. J Immunol 182, 5904-5908, doi:10.4049/jimmunol.0900732 (2009).
23 Picelli, S. et al. Smart-seq2 for sensitive full-length transcriptome profiling in single cells. Nat Methods 10, 1096-1098, doi:10.1038/nmeth.2639 (2013).
24 Lee, Y. et al. Induction and molecular signature of pathogenic TH17 cells. Nat Immunol 13, 991-999, doi:10.1038/ni.2416 (2012).
Velasquez, S., Malik, S., Lutz, S. E., Scemes, E. & Eugenin, E. A. Pannexin1 Channels Are Required for Chemokine-Mediated Migration of CD4+T Lymphocytes: Role in Inflammation and Experimental Autoimmune Encephalomyelitis. J Immunol 196, 4338-4347, doi:10.4049/jimmunol.1502440 (2016).
26 Kurdi, A. T. et al. Tiaml/Rac1 complex controls Il17a transcription and autoimmunity. Nat Commun 7, 13048, doi:10.1038/ncomms13048 (2016).
27 DiSpirito, J. R. et al. Molecular diversification of regulatory T cells in nonlymphoid tissues. Sci Immunol 3, doi:10.1126/sciimmunol.aat5861 (2018).
28 Masopust, D. & Soerens, A. G. Tissue-Resident T Cells and Other Resident Leukocytes. Annu Rev Immunol 37, 521-546, doi:10.1146/annurev-immunol-042617-053214 (2019).
29 Carlson, C. M. et al. Kruppel-like factor 2 regulates thymocyte and T-cell migration. Nature 442, 299-302, doi:10.1038/nature04882 (2006).
30 Bonecchi, R. et al. Differential expression of chemokine receptors and chemotactic responsiveness of type 1 T helper cells (Th1s) and Th2s. J Exp Med 187, 129-134, doi:10.1084/jem.187.1.129 (1998).
31 Singh, U. P., Singh, S., Taub, D. D. & Lillard, J. W., Jr. Inhibition of IFN-gamma-inducible protein-10 abrogates colitis in IL-10−/− mice. J Immunol 171, 1401-1406, doi:10.4049/jimmunol.171.3.1401 (2003).
32 Khor, B., Gardet, A. & Xavier, R. J. Genetics and pathogenesis of inflammatory bowel disease. Nature 474, 307-317, doi:10.1038/nature10209 (2011).
33 Annacker, O. et al. Essential role for CD103 in the T cell-mediated regulation of experimental colitis. J Exp Med 202, 1051-1061, doi:10.1084/jem.20040662 (2005).
34 Lamb, C. A. et al. alphaEbeta7 Integrin Identifies Subsets of Pro-Inflammatory Colonic CD4+T Lymphocytes in Ulcerative Colitis. J Crohns Colitis 11, 610-620, doi:10.1093/ecco-jcc/jjw189 (2017).
35 Johansson-Lindbom, B. & Agace, W. W. Generation of gut-homing T cells and their localization to the small intestinal mucosa. Immunol Rev 215, 226-242, doi:10.1111/j.1600-065X.2006.00482.x (2007).
36 Schneider, M. et al. CD38 is expressed on inflammatory cells of the intestine and promotes intestinal inflammation. PLoS One 10, e0126007, doi:10.1371/journal.pone.0126007 (2015).
37 Abadia-Molina, A. C. et al. CD48 controls T-cell and antigen-presenting cell functions in experimental colitis. Gastroenterology 130, 424-434, doi:10.1053/j.gastro.2005.12.009 (2006).
38 Jackson, R. et al. The translation of non-canonical open reading frames controls mucosal immunity. Nature 564, 434-438, doi:10.1038/s41586-018-0794-7 (2018).
39 Anumanthan, A. et al. Cloning of BY55, a novel Ig superfamily member expressed on NK cells, CTL, and intestinal intraepithelial lymphocytes. J Immunol 161, 2780-2790 (1998).
40 Zhang, Q. & Vignali, D. A. Co-stimulatory and Co-inhibitory Pathways in Autoimmunity. Immunity 44, 1034-1051, doi:10.1016/j.immuni.2016.04.017 (2016).
41 Dardalhon, V. et al. CD226 is specifically expressed on the surface of Th1 cells and regulates their expansion and effector functions. J Immunol 175, 1558-1565, doi:10.4049/jimmunol.175.3.1558 (2005).
42 Gilfillan, S. et al. DNAM-1 promotes activation of cytotoxic lymphocytes by nonprofessional antigen-presenting cells and tumors. J Exp Med 205, 2965-2973, doi:10.1084/jem.20081752 (2008).
43 Ito, R. et al. Interferon-gamma is causatively involved in experimental inflammatory bowel disease in mice. Clin Exp Immunol 146, 330-338, doi:10.1111/j.1365-2249.2006.03214.x (2006).
44 Brass, A. L. et al. The IFITM proteins mediate cellular resistance to influenza A H1N1 virus, West Nile virus, and dengue virus. Cell 139, 1243-1254, doi:10.1016/j.cell.2009.12.017 (2009).
45 Roman, J. et al. Evaluation of responsive gene expression as a sensitive and specific biomarker in patients with ulcerative colitis. Inflamm Bowel Dis 19, 221-229, doi:10.1002/ibd.23020 (2013).
46 Laudato, S. et al. P53-induced miR-30e-5p inhibits colorectal cancer invasion and metastasis by targeting ITGA6 and ITGB1. Int J Cancer 141, 1879-1890, doi:10.1002/ijc.30854 (2017).
47 Sands, B. E. et al. Vedolizumab versus Adalimumab for Moderate-to-Severe Ulcerative Colitis. N Engl J Med 381, 1215-1226, doi:10.1056/NEJMoa1905725 (2019).
48 Mailand, N. & Diffley, J. F. CDKs promote DNA replication origin licensing in human cells by protecting Cdc6 from APC/C-dependent proteolysis. Cell 122, 915-926, doi:10.1016/j.cell.2005.08.013 (2005).
49 Yu, H. Cdc20: a WD40 activator for a cell cycle degradation machine. Mol Cell 27, 3-16, doi:10.1016/j.molcel.2007.06.009 (2007).
50 Forster, R., Davalos-Misslitz, A. C. & Rot, A. CCR7 and its ligands: balancing immunity and tolerance. Nat Rev Immunol 8, 362-371, doi:10.1038/nri2297 (2008).
51 Weder, B. et al. BCL-2 levels do not predict azathioprine treatment response in inflammatory bowel disease, but inhibition induces lymphocyte apoptosis and ameliorates colitis in mice. Clin Exp Immunol 193, 346-360, doi:10.1111/cei.13151 (2018).
52 Gruarin, P. et al. Eomesodermin controls a unique differentiation program in human IL-10 and IFN-gamma coproducing regulatory T cells. Eur J Immunol 49, 96-111, doi:10.1002/eji.201847722 (2019).
53 Zhang, P. et al. Eomesodermin promotes the development of type 1 regulatory T (TR1) cells. Sci Immunol 2, doi:10.1126/sciimmunol.aah7152 (2017).
54 Alfen, J. S. et al. Intestinal IFN-gamma-producing type 1 regulatory T cells coexpress CCR5 and programmed cell death protein 1 and downregulate IL-10 in the inflamed guts of patients with inflammatory bowel disease. J Allergy Clin Immunol 142, 1537-1547 e1538, doi:10.1016/j.jaci.2017.12.984 (2018).
55 Amarnath, S. et al. The PDL1-PD1 axis converts human TH1 cells into regulatory T cells. Sci Transl Med 3, 111ra120, doi:10.1 126/scitranslmed.3003130 (2011).
56 Takahashi, S. et al. De novo and rare mutations in the HSPA1L heat shock gene associated with inflammatory bowel disease. Genome Med 9, 8, doi:10.1186/si3073-016-0394-9 (2017).
57 van Eden, W., van der Zee, R. & Prakken, B. Heat-shock proteins induce T-cell regulation of chronic inflammation. Nat Rev Immunol 5, 318-330, doi:10.1038/nri1593 (2005).
58 de Lange, K. M. et al. Genome-wide association study implicates immune activation of multiple integrin genes in inflammatory bowel disease. Nat Genet 49, 256-261, doi:10.1038/ng.3760 (2017).
59 Huang, H. et al. Fine-mapping inflammatory bowel disease loci to single-variant resolution. Nature 547, 173-178, doi:10.1038/nature22969 (2017).
60 Liu, J. Z. et al. Association analyses identify 38 susceptibility loci for inflammatory bowel disease and highlight shared genetic risk across populations. Nat Genet 47, 979-986, doi:10.1038/ng.3359 (2015).
61 Jostins, L. et al. Host-microbe interactions have shaped the genetic architecture of inflammatory bowel disease. Nature 491, 119-124, doi:10.1038/nature11582 (2012).
62 Kakiuchi, N. et al. Frequent mutations that converge on the NFKBIZ pathway in ulcerative colitis. Nature 577, 260-265, doi:10.1038/s41586-019-1856-1 (2020).
63 Grenningloh, R., Kang, B. Y. & Ho, I. C. Ets-1, a functional cofactor of T-bet, is essential for Th1 inflammatory responses. J Exp Med 201, 615-626, doi:10.1084/jem.20041330 (2005).
64 Wells, M. L., Perera, L. & Blackshear, P. J. An Ancient Family of RNA-Binding Proteins: Still Important! Trends Biochem Sci 42, 285-296, doi:10.1016/j.tibs.2016.12.003 (2017).
65 Wang, X., Sumida, H. & Cyster, J. G. GPR18 is required for a normal CD8alphaalpha intestinal intraepithelial lymphocyte compartment. J Exp Med 211, 2351-2359, doi:10.1084/jem.20140646 (2014).
66 Tan, C. L. et al. CD160 Stimulates CD8(+) T Cell Responses and Is Required for Optimal Protective Immunity to Listeria monocytogenes. Immunohorizons 2, 238-250, doi:10.4049/immunohorizons.1800039 (2018).
67 Tu, T. C. et al. CD160 is essential for NK-mediated IFN-gamma production. J Exp Med 212, 415-429, doi:10.1084/jem.20131601 (2015).
68 Korn, T. et al. IL-21 initiates an alternative pathway to induce proinflammatory T(H)17 cells. Nature 448, 484-487, doi:10.1038/nature05970 (2007).
69 Zhou, L. et al. IL-6 programs T(H)-17 cell differentiation by promoting sequential engagement of the IL-21 and IL-23 pathways. Nature immunology 8, 967-974, doi:10.1038/ni1488 (2007).
70 Hsu, C. Y. et al. SUMO-defective c-Maf preferentially transactivates 1121 to exacerbate autoimmune diabetes. J Clin Invest 128, 3779-3793, doi:10.1172/JCI98786 (2018).
71 Sato, K. et al. Marked induction of c-Maf protein during Th17 cell differentiation and its implication in memory Th cell development. J Biol Chem 286, 14963-14971, doi:10.1074/jbc.M111.218867 (2011).
72 Iwamoto, T. et al. Interleukin-21-producing c-Maf-expressing CD4+ T cells induce effector CD8+ T cells and enhance autoimmune inflammation in scurfy mice. Arthritis Rheumatol 66, 2079-2090, doi:10.1002/art.38658 (2014).
73 Acosta-Rodriguez, E. V., Napolitani, G., Lanzavecchia, A. & Sallusto, F. Interleukins 1beta and 6 but not transforming growth factor-beta are essential for the differentiation of interleukin 17-producing human T helper cells. Nature immunology 8, 942-949, doi:10.1038/ni1496 (2007).
74 Becattini, S. et al. T cell immunity. Functional heterogeneity of human memory CD4(+) T cell clones primed by pathogens or vaccines. Science 347, 400-406, doi:10.1 126/science.1260668 (2015).
75 Gagliani, N. et al. Th17 cells transdifferentiate into regulatory T cells during resolution of inflammation. Nature 523, 221-225, doi:10.1038/nature14452 (2015).
76 Wu, C. et al. SGK1 Governs the Reciprocal Development of Th17 and Regulatory T Cells. Cell Rep 22, 653-665, doi:10.1016/j.celrep.2017.12.068 (2018).
77 Uniken Venema, W. T. et al. Single-Cell RNA Sequencing of Blood and Ileal T Cells From Patients With Crohn's Disease Reveals Tissue-Specific Characteristics and Drug Targets. Gastroenterology 156, 812-815 e822, doi:10.1053/j.gastro.2018.10.046 (2019).
78 Fuchs, A. et al. Intraepithelial type 1 innate lymphoid cells are a unique subset of IL-12- and IL-15-responsive IFN-gamma-producing cells. Immunity 38, 769-781, doi:10.1016/j.immuni.2013.02.010 (2013).
79 Atarashi, K. et al. Ectopic colonization of oral bacteria in the intestine drives TH1 cell induction and inflammation. Science 358, 359-365, doi:10.1126/science.aan4526 (2017).
80 Bolger, A. M., Lohse, M. & Usadel, B. Trimmomatic: a flexible trimmer for Illumina sequence data. Bioinformatics 30, 2114-2120, doi:10.1093/bioinformatics/btul70 (2014).
81 Langmead, B. & Salzberg, S. L. Fast gapped-read alignment with Bowtie 2. Nat Methods 9, 357-359, doi:10.1038/nmeth. 1923 (2012).
82 Li, B. & Dewey, C. N. RSEM: accurate transcript quantification from RNA-Seq data with or without a reference genome. BMC Bioinformatics 12, 323, doi:10.1186/1471-2105-12-323 (2011).
83 Kowalczyk, M. S. et al. Single-cell RNA-seq reveals changes in cell cycle and differentiation programs upon aging of hematopoietic stem cells. Genome Res 25, 1860-1872, doi:10.1101/gr.192237.115 (2015).
84 Finak, G. et al. MAST: a flexible statistical framework for assessing transcriptional changes and characterizing heterogeneity in single-cell RNA sequencing data. Genome Biol 16, 278, doi:10.1186/s13059-015-0844-5 (2015).
85 van der Maaten, L. Accelerating t-SNE using Tree-Based Algorithms. Journal of Machine Learning Research 15, 3221-3245 (2014).
86 Lopez, R., Regier, J., Cole, M. B., Jordan, M. I. & Yosef, N. Deep generative modeling for single-cell transcriptomics. Nat Methods 15, 1053-1058, doi:10.1038/s41592-018-0229-2 (2018).
87 Wolf, F. A., Angerer, P. & Theis, F. J. SCANPY: large-scale single-cell gene expression data analysis. Genome Biol 19, 15, doi:10.1186/s13059-017-1382-0 (2018).
88 Becht, E. et al. Dimensionality reduction for visualizing single-cell data using UMAP. Nat Biotechnol, doi:10.1038/nbt.4314 (2018).
89 Robinson, M. D., McCarthy, D. J. & Smyth, G. K. edgeR: a Bioconductor package for differential expression analysis of digital gene expression data. Bioinformatics 26, 139-140, doi:10.1093/bioinformatics/btp616 (2010).
90 DeTomaso, D. et al. Functional interpretation of single cell similarity maps. Nat Commun 10, 4376, doi:10.1038/s41467-019-12235-0 (2019).

### Tables

Table 1A-1D. Tables related to the Smart-Seq2, in-vitro T cell cytokine stimulation experiments. Includes the results of differential expression comparisons (GFP+ vs GFP− comparisons along with the results of the combined, two-factor model.

Table Heading Descriptions:

Each section represents the output of MAST in computing a differential expression test (See Methods text for full details).

Ctrl GFP+vs GFP−: GFP coefficient (GFP+vs GFP−), only Ctrl cells

KO GFP+vs GFP−: GFP coefficient (GFP+vs GFP−), only KO cells

Combined Model: Analysis of the interaction term (GFP:Genotype) in a combined model with both Ctrl and KO cells.

Coefficient tested is for GFP+:GenotypeCtrl

Th1: Cells stimulated in Th1-polarizing conditions (IL12+IL21+IL23)

Th17: Cells stimulated in Th17-polarizing conditions (IL1+IL6+IL23)

Descriptions for MAST (common) to all sections:

Gene: Gene for which the test was computed

coefC: continuous coefficient in the MAST hurdle model

pvalC: p-value associated with the continuous coefficient

coefD: discrete coefficient in the MAST hurdle model

pvalD: p-value associated with the discrete coefficient

pvalH: overall p-value associated with the MAST hurdle model

log FC: log 2 effect size associated with the tested coefficient

FDR. Benjamini-Hochberg False Discovery Rate

TABLE 1A
Th1 Ctrl GFP+ vs GFP−

| Gene | coefC | pvalC | coefD | pvalD | pvalH | logFC | FDR |
|---|---|---|---|---|---|---|---|
| IL23R | 1.61 | 2.41E−05 | 4.54E+00 | 5.84E−27 | 1.06E−29 | 4.76 | 1.38E−25 |
| EGFP_IRES_IL23R | 2.40 | 3.81E−04 | 4.26E+00 | 1.69E−25 | 4.04E−27 | 3.81 | 2.62E−23 |
| RPS2 | 1.00 | 2.35E−19 | −1.20E−16 | 1.00E+00 | 2.68E−18 | 1.00 | 1.16E−14 |
| H2-T23 | −0.96 | 9.22E−19 | −1.20E−16 | 1.00E+00 | 1.03E−17 | −0.96 | 3.35E−14 |
| CBX3 | −0.77 | 1.15E−14 | −1.20E−16 | 1.00E+00 | 1.13E−13 | −0.77 | 2.44E−10 |
| VAPA | 1.69 | 3.39E−14 | 1.84E+00 | 1.21E−01 | 9.86E−14 | 1.75 | 2.44E−10 |
| IL4 | −3.47 | 2.74E−03 | −2.97E+00 | 5.21E−12 | 5.17E−13 | −3.13 | 9.58E−10 |
| RPSA | −0.46 | 6.37E−14 | −1.20E−16 | 1.00E+00 | 6.09E−13 | −0.46 | 9.87E−10 |
| RNF187 | 1.72 | 4.02E−11 | 2.07E+00 | 4.33E−04 | 6.96E−13 | 2.44 | 1.00E−09 |
| ARGLU1 | −1.05 | 1.14E−13 | −1.20E−16 | 1.00E+00 | 1.08E−12 | −1.04 | 1.40E−09 |
| CLASP2 | 1.86 | 1.02E−11 | 1.61E+00 | 1.25E−02 | 3.94E−12 | 2.05 | 4.65E−09 |
| RPS12 | −0.49 | 7.22E−13 | −1.20E−16 | 1.00E+00 | 6.61E−12 | −0.49 | 7.14E−09 |
| TPT1 | −0.43 | 1.48E−12 | −1.20E−16 | 1.00E+00 | 1.34E−11 | −0.43 | 1.34E−08 |
| LARS2 | 0.60 | 7.74E−12 | −1.20E−16 | 1.00E+00 | 6.77E−11 | 0.60 | 6.27E−08 |
| ANXA2 | 1.11 | 1.41E−11 | 7.73E−01 | 6.73E−01 | 1.12E−10 | 1.12 | 9.67E−08 |
| SMOX | 1.55 | 3.89E−11 | 9.62E−01 | 1.97E−01 | 1.43E−10 | 1.78 | 1.16E−07 |
| RPL12 | −0.39 | 2.95E−11 | −1.20E−16 | 1.00E+00 | 2.51E−10 | −0.39 | 1.92E−07 |
| MAP3K1 | −1.20 | 2.66E−10 | −1.66E+00 | 4.43E−02 | 2.85E−10 | −1.27 | 2.05E−07 |
| RNH1 | 0.80 | 9.93E−11 | 1.20E+00 | 3.94E−01 | 5.73E−10 | 0.84 | 3.91E−07 |
| RPS20 | −0.39 | 7.32E−11 | −1.20E−16 | 1.00E+00 | 6.11E−10 | −0.39 | 3.96E−07 |
| RPL5 | −0.31 | 9.11E−11 | −1.20E−16 | 1.00E+00 | 7.57E−10 | −0.31 | 4.67E−07 |
| EIF3F | 0.76 | 1.14E−10 | −1.20E−16 | 1.00E+00 | 9.41E−10 | 0.76 | 5.54E−07 |
| PDCD4 | −1.10 | 3.18E−08 | −2.96E+00 | 1.14E−03 | 1.14E−09 | −1.29 | 6.45E−07 |
| NSUN2 | 1.15 | 2.83E−10 | −7.08E−01 | 2.65E−01 | 1.23E−09 | 0.88 | 6.64E−07 |
| TIMP1 | 1.02 | 8.98E−02 | 2.06E+00 | 1.41E−09 | 2.60E−09 | 4.05 | 1.35E−06 |
| 1110008F13RIK | 1.43 | 4.97E−07 | 1.18E+00 | 2.86E−04 | 4.50E−09 | 2.27 | 2.24E−06 |
| PHLDA3 | 1.09 | 1.14E−03 | 1.69E+00 | 2.26E−07 | 7.61E−09 | 2.49 | 3.53E−06 |
| ACTB | −0.45 | 9.71E−10 | −1.20E−16 | 1.00E+00 | 7.63E−09 | −0.45 | 3.53E−06 |
| RNR1 | 0.90 | 1.61E−09 | −1.20E−16 | 1.00E+00 | 1.25E−08 | 0.90 | 5.48E−06 |
| S1PR1 | 1.16 | 9.45E−07 | 1.74E+00 | 4.45E−04 | 1.27E−08 | 2.07 | 5.48E−06 |
| ANP32B | 1.20 | 5.36E−09 | −1.87E+00 | 1.36E−01 | 1.33E−08 | 1.13 | 5.56E−06 |
| PSMB9 | −0.67 | 2.45E−09 | −1.20E−16 | 1.00E+00 | 1.88E−08 | −0.67 | 7.63E−06 |
| SLC4A11 | 1.32 | 1.86E−08 | 8.08E−01 | 4.93E−02 | 1.95E−08 | 1.86 | 7.68E−06 |
| PELI1 | 1.22 | 9.77E−07 | 2.00E+00 | 1.38E−03 | 3.75E−08 | 1.78 | 1.43E−05 |
| HSH2D | −0.80 | 1.29E−01 | −2.16E+00 | 2.04E−08 | 4.66E−08 | −1.36 | 1.72E−05 |
| LSM4 | 0.93 | 1.41E−08 | 1.48E+00 | 2.47E−01 | 5.29E−08 | 0.97 | 1.91E−05 |
| RPL30 | −0.36 | 7.69E−09 | −1.20E−16 | 1.00E+00 | 5.72E−08 | −0.36 | 1.95E−05 |
| H2-Q10 | 1.07 | 4.40E−05 | 1.60E+00 | 4.35E−05 | 5.58E−08 | 2.50 | 1.95E−05 |
| UCK2 | 1.31 | 6.31E−08 | 1.63E+00 | 4.46E−02 | 5.88E−08 | 1.60 | 1.95E−05 |
| RPS27A | −0.28 | 1.08E−08 | −1.20E−16 | 1.00E+00 | 7.97E−08 | −0.28 | 2.58E−05 |
| KLF6 | 1.20 | 8.41E−08 | 1.07E+00 | 5.25E−02 | 8.90E−08 | 1.59 | 2.81E−05 |
| CALM2 | 0.71 | 1.35E−08 | 5.59E−01 | 8.74E−01 | 9.78E−08 | 0.72 | 3.02E−05 |
| SRM | 1.24 | 1.51E−08 | −1.45E−02 | 9.37E−01 | 1.10E−07 | 1.12 | 3.31E−05 |
| ENSA | −1.06 | 2.93E−05 | −1.63E+00 | 1.55E−04 | 1.26E−07 | −1.82 | 3.72E−05 |
| CANX | −0.55 | 2.07E−08 | −1.20E−16 | 1.00E+00 | 1.50E−07 | −0.55 | 4.31E−05 |
| PAXBP1 | −1.00 | 1.08E−05 | −1.57E+00 | 6.42E−04 | 1.84E−07 | −1.54 | 5.19E−05 |
| CLPTM1 | 1.05 | 1.60E−05 | 1.36E+00 | 4.70E−04 | 2.01E−07 | 1.73 | 5.54E−05 |
| DUSP2 | 1.18 | 7.41E−04 | 1.64E+00 | 1.14E−05 | 2.22E−07 | 2.64 | 5.99E−05 |
| NPTN | −1.09 | 2.12E−05 | −1.43E+00 | 5.90E−04 | 3.24E−07 | −1.66 | 8.39E−05 |
| OST4 | −0.93 | 7.14E−08 | −1.29E+00 | 3.51E−01 | 3.22E−07 | −0.96 | 8.39E−05 |
| TMEM161B | −1.16 | 6.96E−05 | −1.21E+00 | 2.35E−04 | 4.23E−07 | −0.96 | 1.07E−04 |
| CSF1 | 0.26 | 6.40E−01 | 1.96E+00 | 6.85E−08 | 4.29E−07 | 1.23 | 1.07E−04 |
| RBMS1 | 0.89 | 4.12E−04 | 1.87E+00 | 4.78E−05 | 5.02E−07 | 1.90 | 1.23E−04 |
| GM5111 | | 1.00E+00 | 3.79E+00 | 5.48E−07 | 5.48E−07 | | 1.32E−04 |
| ACTR2 | −0.57 | 9.10E−08 | −1.20E−16 | 1.00E+00 | 6.30E−07 | −0.57 | 1.48E−04 |
| S100A10 | 0.66 | 9.31E−08 | −1.20E−16 | 1.00E+00 | 6.43E−07 | 0.66 | 1.49E−04 |
| CASP6 | 0.94 | 1.18E−07 | −4.16E−01 | 6.84E−01 | 7.43E−07 | 0.93 | 1.69E−04 |
| ID2 | −1.20 | 8.17E−07 | −8.88E−01 | 5.25E−02 | 8.00E−07 | −1.69 | 1.76E−04 |
| TMEM30A | 0.86 | 7.60E−05 | 3.04E+00 | 4.20E−04 | 7.92E−07 | 1.38 | 1.76E−04 |
| RALA | 0.64 | 1.56E−04 | 1.97E+00 | 2.37E−04 | 9.11E−07 | 1.52 | 1.97E−04 |
| RN7S1 | 0.55 | 1.10E−02 | 1.46E+00 | 4.22E−06 | 1.00E−06 | 1.68 | 2.03E−04 |
| RN7S2 | 0.55 | 1.10E−02 | 1.46E+00 | 4.22E−06 | 1.00E−06 | 1.68 | 2.03E−04 |
| RPL7A | −0.27 | 1.41E−07 | −1.20E−16 | 1.00E+00 | 9.63E−07 | −0.27 | 2.03E−04 |
| EMB | 0.78 | 1.49E−07 | 5.59E−01 | 8.74E−01 | 1.00E−06 | 0.79 | 2.03E−04 |
| RORA | 0.71 | 1.68E−02 | .49E+00 | 3.80E−06 | 1.31E−06 | 1.35 | 2.62E−04 |
| RPS21 | −0.33 | 2.03E−07 | −1.20E−16 | 1.00E+00 | 1.37E−06 | −0.33 | 2.69E−04 |
| IZUMO1R | −1.27 | 8.34E−06 | −8.83E−01 | 8.08E−03 | 1.46E−06 | −2.15 | 2.82E−04 |
| MAPK1 | 0.96 | 1.95E−05 | 1.46E+00 | 3.78E−03 | 1.65E−06 | 1.52 | 3.14E−04 |
| RORC | 0.35 | 5.10E−01 | 1.72E+00 | 3.40E−07 | 1.81E−06 | 1.54 | 3.39E−04 |
| ZFP622 | 1.36 | 4.16E−07 | 2.86E−01 | 3.77E−01 | 1.85E−06 | 0.96 | 3.39E−04 |
| RPS15 | −0.26 | 2.76E−07 | −1.20E−16 | 1.00E+00 | 1.84E−06 | −0.26 | 3.39E−04 |
| OSTF1 | 1.03 | 9.09E−07 | 1.19E+00 | 1.81E−01 | 2.37E−06 | 1.24 | 4.27E−04 |
| GATA3 | −0.93 | 7.00E−03 | −1.87E+00 | 2.01E−05 | 2.97E−06 | −2.13 | 5.28E−04 |
| ERMN | 0.94 | 7.85E−03 | 1.87E+00 | 2.09E−05 | 3.42E−06 | 2.39 | 5.99E−04 |
| TPR | 0.76 | 1.71E−06 | 1.26E+00 | 1.39E−01 | 3.57E−06 | 0.80 | 6.18E−04 |
| PPP1R16B | −0.92 | 2.35E−05 | −1.80E+00 | 7.54E−03 | 3.68E−06 | −1.12 | 6.28E−04 |
| PRRC2A | 0.60 | 6.75E−06 | 2.27E+00 | 3.52E−02 | 4.34E−06 | 0.65 | 7.31E−04 |
| PRKCH | 0.97 | 1.17E−06 | 5.94E−01 | 3.08E−01 | 4.42E−06 | 1.17 | 7.35E−04 |
| HSD17B10 | 0.72 | 1.00E−06 | −6.85E−01 | 5.01E−01 | 5.08E−06 | 0.64 | 8.34E−04 |
| RPLP2 | −0.26 | 8.06E−07 | −1.20E−16 | 1.00E+00 | 5.18E−06 | −0.26 | 8.39E−04 |
| WNT10B | 0.21 | 8.48E−01 | 2.38E+00 | 8.43E−07 | 5.30E−06 | 0.87 | 8.49E−04 |
| RPL28 | −0.26 | 8.74E−07 | −1.20E−16 | 1.00E+00 | 5.59E−06 | −0.26 | 8.84E−04 |
| SLC29A2 | 1.16 | 1.12E−02 | 1.40E+00 | 2.58E−05 | 5.72E−06 | 1.54 | 8.93E−04 |
| PDE4B | 1.24 | 6.38E−05 | 1.17E+00 | 4.40E−03 | 5.85E−06 | 1.87 | 9.03E−04 |
| INHBA | 0.59 | 2.70E−01 | 1.60E+00 | 1.96E−06 | 6.63E−06 | 2.84 | 1.01E−03 |
| ATP6V0D2 | 0.35 | 3.06E−01 | 1.57E+00 | 1.94E−06 | 7.14E−06 | 2.43 | 1.08E−03 |
| PSME1 | −0.38 | 1.21E−06 | −1.20E−16 | 1.00E+00 | 7.63E−06 | −0.38 | 1.14E−03 |
| RBM17 | 0.82 | 1.45E−06 | 1.04E+00 | 5.84E−01 | 7.81E−06 | 0.83 | 1.15E−03 |
| TMEM160 | 0.80 | 3.46E−04 | 1.33E+00 | 1.09E−03 | 8.02E−06 | 1.76 | 1.17E−03 |
| LAMA5 | −0.75 | 2.70E−04 | −1.48E+00 | 1.50E−03 | 8.51E−06 | −1.25 | 1.20E−03 |
| ITSN2 | 1.33 | 1.34E−06 | −8.48E−03 | 9.63E−01 | 8.43E−06 | 0.96 | 1.20E−03 |
| CALM1 | 0.51 | 1.65E−06 | −6.40E−01 | 5.31E−01 | 8.46E−06 | 0.48 | 1.20E−03 |
| GGNBP2 | 1.13 | 1.46E−06 | 2.60E−01 | 7.22E−01 | 8.61E−06 | 1.14 | 1.20E−03 |
| PPP4C | 0.77 | 5.51E−06 | 1.87E+00 | 1.18E−01 | 9.66E−06 | 0.89 | 1.33E−03 |
| IFI27L2A | −0.74 | 3.38E−06 | −1.60E+00 | 2.21E−01 | 9.72E−06 | −0.78 | 1.33E−03 |
| YIPF4 | 0.76 | 1.23E−05 | 1.63E+00 | 4.93E−02 | 1.02E−05 | 1.07 | 1.38E−03 |
| IIGP1 | −0.69 | 3.46E−02 | −1.38E+00 | 1.79E−05 | 1.08E−05 | −1.99 | 1.44E−03 |
| UTP18 | 1.43 | 2.29E−06 | 1.96E−01 | 5.73E−01 | 1.21E−05 | 1.14 | 1.59E−03 |
| D030056L22RIK | 1.48 | 3.74E−03 | 1.46E+00 | 1.65E−04 | 1.24E−05 | 1.11 | 1.59E−03 |
| CCDC71L | 0.78 | 9.23E−03 | 1.30E+00 | 6.99E−05 | 1.24E−05 | 1.23 | 1.59E−03 |
| USP39 | 0.78 | 6.80E−05 | 1.53E+00 | 9.48E−03 | 1.24E−05 | 1.37 | 1.59E−03 |
| GTF2A1 | −1.08 | 2.05E−06 | 6.32E−02 | 8.89E−01 | 1.26E−05 | −0.77 | 1.60E−03 |
| BHLHE41 | −0.10 | 2.24E−01 | −1.46E+00 | 4.50E−06 | 1.29E−05 | −0.25 | 1.62E−03 |
| SUMF1 | 1.80 | 1.20E−04 | 9.46E−01 | 5.61E−03 | 1.32E−05 | 1.03 | 1.65E−03 |
| LAMP1 | 0.97 | 5.72E−05 | 1.09E+00 | 1.31E−02 | 1.40E−05 | 1.52 | 1.73E−03 |
| PMM1 | 0.58 | 4.63E−03 | 1.78E+00 | 1.60E−04 | 1.46E−05 | 2.07 | 1.78E−03 |
| IL27RA | −1.08 | 1.27E−05 | −6.05E−01 | 7.93E−02 | 1.56E−05 | −1.29 | 1.90E−03 |
| STOM | 0.83 | 5.92E−02 | 1.43E+00 | 1.69E−05 | 1.61E−05 | 1.74 | 1.93E−03 |
| TUSC2 | −0.02 | 9.49E−01 | 1.55E+00 | 2.68E−06 | 1.64E−05 | 1.83 | 1.95E−03 |
| SOCS3 | 0.77 | 1.61E−03 | 2.35E+00 | 5.21E−04 | 1.68E−05 | 1.35 | 1.98E−03 |
| SYNE2 | 0.46 | 3.44E−01 | 2.11E+00 | 4.47E−06 | 1.72E−05 | 0.35 | 2.01E−03 |
| TSPO | 0.48 | 2.87E−06 | −1.20E−16 | 1.00E+00 | 1.75E−05 | 0.48 | 2.03E−03 |
| 6430571L13RIK | −0.88 | 1.73E−02 | −1.31E+00 | 5.67E−05 | 1.77E−05 | −1.36 | 2.03E−03 |
| CCDC85B | 0.37 | 8.41E−02 | 1.72E+00 | 1.41E−05 | 1.80E−05 | 1.12 | 2.05E−03 |
| RAD23A | −0.82 | 8.25E−06 | −1.07E+00 | 1.80E−01 | 1.97E−05 | −0.94 | 2.22E−03 |
| PLBD2 | 0.57 | 2.00E−02 | 1.44E+00 | 5.74E−05 | 2.04E−05 | 1.74 | 2.26E−03 |
| PTPN1 | 0.80 | 3.36E−06 | −1.20E−16 | 1.00E+00 | 2.04E−05 | 0.79 | 2.26E−03 |
| PERP | 0.69 | 8.58E−03 | 1.38E+00 | 1.36E−04 | 2.18E−05 | 2.20 | 2.39E−03 |
| RPS13 | −0.21 | 3.68E−06 | −1.20E−16 | 1.00E+00 | 2.23E−05 | −0.21 | 2.42E−03 |
| ODC1 | 1.03 | 5.80E−06 | −5.18E−01 | 4.48E−01 | 2.58E−05 | 0.89 | 2.78E−03 |
| IL22 | | 1.00E+00 | 3.31E+00 | 2.59E−05 | 2.59E−05 | | 2.78E−03 |
| RPS26 | −0.22 | 4.42E−06 | −1.20E−16 | 1.00E+00 | 2.66E−05 | −0.22 | 2.81E−03 |
| TRP53I11 | −0.44 | 4.95E−02 | −1.51E+00 | 3.35E−05 | 2.67E−05 | −1.73 | 2.81E−03 |
| BLOC1S4 | 0.60 | 1.32E−01 | 1.47E+00 | 1.50E−05 | 2.73E−05 | 1.51 | 2.86E−03 |
| CFL1 | 0.26 | 4.65E−06 | −1.20E−16 | 1.00E+00 | 2.79E−05 | 0.26 | 2.89E−03 |
| SERBP1 | 0.36 | 5.21E−06 | −1.20E−16 | 1.00E+00 | 3.11E−05 | 0.36 | 3.20E−03 |
| MFHAS1 | 0.88 | 3.56E−05 | 9.40E−01 | 5.66E−02 | 3.16E−05 | 1.11 | 3.22E−03 |
| NOP9 | 0.71 | 1.41E−03 | 1.34E+00 | 1.20E−03 | 3.22E−05 | 1.60 | 3.27E−03 |
| IL18R1 | 0.90 | 2.67E−02 | 1.27E+00 | 7.26E−05 | 3.27E−05 | 2.03 | 3.29E−03 |
| STX6 | 0.71 | 9.73E−03 | 1.62E+00 | 1.92E−04 | 3.37E−05 | 1.72 | 3.36E−03 |
| CSGALNACT1 | 0.04 | 9.53E−01 | 1.99E+00 | 5.75E−06 | 3.41E−05 | 0.82 | 3.37E−03 |
| TPD52L2 | 0.67 | 1.07E−05 | 1.42E+00 | 3.15E−01 | 3.73E−05 | 0.71 | 3.67E−03 |
| PLAGL2 | 0.66 | 9.73E−06 | 1.23E+00 | 3.79E−01 | 3.84E−05 | 0.69 | 3.72E−03 |
| RACK1 | −0.25 | 6.51E−06 | −1.20E−16 | 1.00E+00 | 3.84E−05 | −0.25 | 3.72E−03 |
| KARS | −0.58 | 7.74E−06 | −1.20E−16 | 1.00E+00 | 4.54E−05 | −0.58 | 4.36E−03 |
| RBM43 | −0.65 | 4.35E−03 | −1.10E+00 | 5.87E−04 | 4.66E−05 | −1.67 | 4.44E−03 |
| RPS10 | −0.22 | 8.08E−06 | −1.20E−16 | 1.00E+00 | 4.73E−05 | −0.22 | 4.48E−03 |
| SCAND1 | 0.44 | 8.89E−02 | 1.33E+00 | 3.73E−05 | 4.77E−05 | 1.70 | 4.48E−03 |
| PGS1 | 0.94 | 1.10E−05 | 5.21E−01 | 4.56E−01 | 4.81E−05 | 1.06 | 4.48E−03 |
| SERPINB5 | 0.73 | 1.04E−01 | 1.35E+00 | 3.32E−05 | 4.85E−05 | 2.06 | 4.49E−03 |
| YWHAH | 0.47 | 8.68E−06 | 5.59E−01 | 8.74E−01 | 5.00E−05 | 0.47 | 4.60E−03 |
| H2−K1 | −0.43 | 9.07E−06 | −1.20E−16 | 1.00E+00 | 5.28E−05 | −0.43 | 4.79E−03 |
| RBM42 | 0.78 | 5.28E−05 | 1.57E+00 | 6.67E−02 | 5.25E−05 | 1.01 | 4.79E−03 |
| EIF4B | −0.43 | 9.16E−06 | −1.20E−16 | 1.00E+00 | 5.33E−05 | −0.43 | 4.80E−03 |
| MTRF1L | 0.69 | 3.64E−02 | 1.44E+00 | 9.34E−05 | 5.43E−05 | 1.05 | 4.86E−03 |
| IFNG | 3.15 | 2.89E−03 | 1.15E+00 | 1.12E−03 | 5.84E−05 | 2.17 | 5.19E−03 |
| RBM3 | −0.38 | 1.02E−05 | −1.20E−16 | 1.00E+00 | 5.88E−05 | −0.38 | 5.19E−03 |
| EEF1A1 | −0.27 | 1.05E−05 | −1.20E−16 | 1.00E+00 | 6.06E−05 | −0.27 | 5.27E−03 |
| TRAPPC6B | 1.14 | 3.41E−05 | 5.74E−01 | 1.33E−01 | 6.02E−05 | 1.40 | 5.27E−03 |
| EIF4A2 | −0.51 | 4.59E−05 | −1.98E+00 | 9.62E−02 | 6.19E−05 | −0.62 | 5.35E−03 |
| LIN7C | −0.84 | 7.57E−05 | 8.60E−01 | 5.73E−02 | 6.52E−05 | −0.23 | 5.60E−03 |
| SDHD | −0.66 | 1.51E−05 | 5.81E−01 | 6.10E−01 | 7.55E−05 | −0.57 | 6.32E−03 |
| TNP2 | −0.73 | 5.20E−03 | −1.14E+00 | 8.27E−04 | 7.53E−05 | −1.89 | 6.32E−03 |
| POLR2M | 0.75 | 1.30E−05 | 9.45E−02 | 9.63E−01 | 7.41E−05 | 0.74 | 6.32E−03 |
| CCNB2 | 0.92 | 2.30E−04 | −8.67E−01 | 1.98E−02 | 7.48E−05 | −0.04 | 6.32E−03 |
| RPGRIP1 | −0.38 | 1.38E−01 | −1.40E+00 | 4.40E−05 | 7.88E−05 | −1.12 | 6.55E−03 |
| FAM49B | 0.67 | 1.80E−05 | 4.80E−01 | 5.01E−01 | 8.09E−05 | 0.71 | 6.64E−03 |
| GPR18 | 0.66 | 1.73E−02 | 1.68E+00 | 2.83E−04 | 8.09E−05 | 1.88 | 6.64E−03 |
| FURIN | 0.99 | 1.27E−04 | −1.88E+00 | 4.37E−02 | 8.50E−05 | 0.75 | 6.93E−03 |
| ATG3 | 0.65 | 1.86E−04 | 9.67E−01 | 2.98E−02 | 8.76E−05 | 1.32 | 6.99E−03 |
| HIST1H2AH | 1.65 | 1.56E−02 | −2.18E+00 | 3.40E−04 | 8.79E−05 | −0.39 | 6.99E−03 |
| SLC15A3 | 1.10 | 7.46E−04 | 1.01E+00 | 6.83E−03 | 8.75E−05 | 1.95 | 6.99E−03 |
| DONSON | 0.77 | 2.96E−02 | 1.19E+00 | 1.87E−04 | 8.75E−05 | 1.48 | 6.99E−03 |
| CD72 | −3.12 | 4.55E−05 | −7.32E−01 | 1.59E−01 | 9.07E−05 | −0.39 | 7.17E−03 |
| SLC30A9 | 0.80 | 7.38E−03 | 1.11E+00 | 7.38E−04 | 9.29E−05 | 1.42 | 7.29E−03 |
| THAP11 | 0.71 | 6.99E−03 | 1.11E+00 | 7.82E−04 | 9.34E−05 | 1.79 | 7.29E−03 |
| RGCC | 0.87 | 3.67E−04 | 1.33E+00 | 1.62E−02 | 9.73E−05 | 1.62 | 7.46E−03 |
| ISG20 | −0.65 | 2.10E−01 | −1.57E+00 | 3.89E−05 | 9.64E−05 | −1.69 | 7.46E−03 |
| ETV6 | −0.75 | 1.73E−05 | 1.75E−01 | 9.31E−01 | 9.74E−05 | −0.70 | 7.46E−03 |
| WIPI2 | 0.41 | 3.60E−02 | 1.92E+00 | 1.76E−04 | 9.78E−05 | 1.30 | 7.46E−03 |
| ANP32A | 0.71 | 6.21E−05 | 9.90E−01 | 1.21E−01 | 9.92E−05 | 0.93 | 7.52E−03 |
| CD81 | 0.66 | 2.23E−05 | 9.84E−01 | 5.28E−01 | 1.02E−04 | 0.67 | 7.70E−03 |
| RIPK2 | 0.78 | 1.51E−02 | 1.18E+00 | 4.29E−04 | 1.06E−04 | 1.86 | 7.88E−03 |
| UNC119B | 0.54 | 1.47E−02 | 2.06E+00 | 4.38E−04 | 1.05E−04 | 1.42 | 7.88E−03 |
| ATP5G2 | −0.33 | 1.91E−05 | −1.20E−16 | 1.00E+00 | 1.08E−04 | −0.33 | 7.97E−03 |
| LATS1 | −1.13 | 2.98E−05 | 2.83E−01 | 3.75E−01 | 1.11E−04 | −0.34 | 8.09E−03 |
| RPL23 | −0.20 | 1.98E−05 | −1.20E−16 | 1.00E+00 | 1.11E−04 | −0.20 | 8.09E−03 |
| RPS16 | −0.20 | 1.98E−05 | −1.20E−16 | 1.00E+00 | 1.11E−04 | −0.20 | 8.09E−03 |
| RPS28 | −0.24 | 1.99E−05 | −1.20E−16 | 1.00E+00 | 1.12E−04 | −0.24 | 8.09E−03 |
| KHNYN | −0.98 | 1.30E−03 | −9.04E−01 | 5.09E−03 | 1.13E−04 | −1.25 | 8.11E−03 |
| PAIP1 | −0.64 | 7.65E−04 | −1.33E+00 | 8.98E−03 | 1.14E−04 | −0.91 | 8.19E−03 |
| NMT1 | −0.88 | 3.44E−05 | −7.53E−01 | 3.43E−01 | 1.20E−04 | −0.97 | 8.54E−03 |
| CDKN1A | 0.83 | 2.20E−05 | −1.67E−01 | 8.66E−01 | 1.21E−04 | 0.81 | 8.58E−03 |
| GAS2L1 | 0.94 | 3.38E−02 | 1.34E+00 | 2.46E−04 | 1.27E−04 | 0.87 | 8.93E−03 |
| SHROOM3 | 1.26 | 3.40E−03 | 1.11E+00 | 2.23E−03 | 1.28E−04 | 0.75 | 8.94E−03 |
| 2310036022RIK | 1.02 | 6.41E−05 | 4.41E−01 | 1.82E−01 | 1.39E−04 | 1.03 | 9.65E−03 |
| CAPZA2 | 0.39 | 2.50E−05 | −1.20E−16 | 1.00E+00 | 1.39E−04 | 0.39 | 9.65E−03 |
| IER2 | 0.81 | 9.37E−05 | 9.22E−01 | 1.17E−01 | 1.42E−04 | 1.20 | 9.81E−03 |
| ATXN2L | 0.78 | 3.23E−05 | 5.88E−01 | 5.49E−01 | 1.48E−04 | 0.80 | 1.02E−02 |
| COX15 | 0.42 | 1.33E−01 | −1.26E+00 | 9.04E−05 | 1.52E−04 | −0.29 | 1.03E−02 |
| PLEK | −0.05 | 8.72E−01 | 1.48E+00 | 2.81E−05 | 1.53E−04 | 1.74 | 1.04E−02 |
| ZDHHC7 | 1.07 | 9.05E−04 | 8.42E−01 | 1.08E−02 | 1.57E−04 | 1.38 | 1.06E−02 |
| TRIM27 | 0.82 | 7.52E−04 | 1.07E+00 | 1.36E−02 | 1.63E−04 | 1.27 | 1.10E−02 |
| RAC1 | 0.92 | 3.66E−05 | 1.04E+00 | 5.84E−01 | 1.72E−04 | 0.93 | 1.15E−02 |
| RAPH1 | 0.42 | 7.36E−02 | 1.31E+00 | 1.72E−04 | 1.74E−04 | 0.98 | 1.15E−02 |
| FBXO2 | 1.25 | 6.97E−03 | 1.22E+00 | 1.53E−03 | 1.73E−04 | 1.02 | 1.15E−02 |
| UBA5 | 1.56 | 4.10E−05 | 2.17E−01 | 4.94E−01 | 1.76E−04 | 0.83 | 1.16E−02 |
| FLOT2 | 0.77 | 3.31E−03 | 1.26E+00 | 3.26E−03 | 1.77E−04 | 1.60 | 1.16E−02 |
| ADIPOR1 | 0.88 | 3.72E−05 | 4.14E−01 | 6.30E−01 | 1.80E−04 | 0.92 | 1.17E−02 |
| LRRFIP2 | 0.91 | 9.21E−04 | 8.00E−01 | 1.30E−02 | 1.88E−04 | 1.14 | 1.22E−02 |
| DAXX | −0.07 | 7.11E−01 | −3.18E+00 | 3.79E−05 | 1.92E−04 | −0.63 | 1.24E−02 |
| ASB2 | −0.30 | 4.51E−01 | −1.34E+00 | 5.49E−05 | 2.20E−04 | −1.57 | 1.41E−02 |
| SOX5 | 0.70 | 1.75E−02 | 1.08E+00 | 8.53E−04 | 2.29E−04 | 1.02 | 1.46E−02 |
| LGALS4 | −1.04 | 3.16E−04 | −6.23E−01 | 5.19E−02 | 2.30E−04 | −0.88 | 1.46E−02 |
| ACBD6 | 0.83 | 8.58E−05 | 5.19E−01 | 2.62E−01 | 2.38E−04 | 1.09 | 1.51E−02 |
| FBXL22 | −2.22 | 4.69E−05 | 1.21E−01 | 7.89E−01 | 2.44E−04 | −0.24 | 1.53E−02 |
| ARPC1B | 0.29 | 4.51E−05 | −1.20E−16 | 1.00E+00 | 2.43E−04 | 0.29 | 1.53E−02 |
| MAPK6 | 0.75 | 4.44E−04 | 1.66E+00 | 3.90E−02 | 2.49E−04 | 0.96 | 1.55E−02 |
| PKIB | 1.03 | 1.04E−01 | 1.57E+00 | 1.98E−04 | 2.63E−04 | 0.56 | 1.63E−02 |
| FZR1 | 0.61 | 1.38E−02 | 1.39E+00 | 1.32E−03 | 2.78E−04 | 1.61 | 1.72E−02 |
| PRKCQ | −0.40 | 8.76E−02 | −1.95E+00 | 2.54E−04 | 2.89E−04 | −1.41 | 1.77E−02 |
| SOD1 | −0.29 | 5.47E−05 | −1.20E−16 | 1.00E+00 | 2.92E−04 | −0.29 | 1.77E−02 |
| ATP6V0B | 0.62 | 3.02E−04 | 1.49E+00 | 7.22E−02 | 2.90E−04 | 0.88 | 1.77E−02 |
| UBASH3A | −0.68 | 1.93E−02 | −1.06E+00 | 1.02E−03 | 2.93E−04 | −1.59 | 1.77E−02 |
| IL2RA | 0.40 | 5.60E−05 | −1.20E−16 | 1.00E+00 | 2.99E−04 | 0.40 | 1.79E−02 |
| PGP | 0.74 | 5.35E−04 | 9.02E−01 | 3.94E−02 | 2.99E−04 | 1.47 | 1.79E−02 |
| NRAS | 0.47 | 5.75E−05 | 4.70E−01 | 9.30E−01 | 3.05E−04 | 0.47 | 1.82E−02 |
| SLAMF6 | −2.48 | 2.26E−04 | −6.96E−01 | 1.08E−01 | 3.06E−04 | −0.78 | 1.82E−02 |
| RAB3GAP1 | −0.83 | 4.38E−04 | −7.37E−01 | 5.15E−02 | 3.11E−04 | −1.15 | 1.83E−02 |
| BATF | 0.71 | 1.25E−04 | 9.24E−01 | 2.29E−01 | 3.10E−04 | 1.00 | 1.83E−02 |
| D17H6S53E | −0.39 | 6.58E−02 | −1.42E+00 | 3.54E−04 | 3.11E−04 | −0.96 | 1.83E−02 |
| PITHD1 | 0.71 | 5.27E−03 | 1.07E+00 | 3.94E−03 | 3.19E−04 | 1.70 | 1.86E−02 |
| CCR3 | | 1.00E+00 | 3.06E+00 | 3.30E−04 | 3.30E−04 | | 1.92E−02 |
| STAT5A | 0.74 | 8.97E−05 | 8.95E−01 | 4.22E−01 | 3.38E−04 | 0.84 | 1.95E−02 |
| MLF2 | 0.54 | 7.43E−05 | −4.14E−01 | 6.27E−01 | 3.47E−04 | 0.49 | 2.00E−02 |
| ATP5E | −0.31 | 6.66E−05 | −1.20E−16 | 1.00E+00 | 3.52E−04 | −0.31 | 2.02E−02 |
| MIDN | 0.82 | 8.04E−05 | 2.29E−01 | 5.75E−01 | 3.59E−04 | 0.74 | 2.05E−02 |
| SLC39A8 | 0.17 | 5.62E−01 | 1.29E+00 | 8.26E−05 | 3.64E−04 | 1.44 | 2.07E−02 |
| RFNG | 0.42 | 1.14E−01 | 1.20E+00 | 2.73E−04 | 3.80E−04 | 1.53 | 2.15E−02 |
| TMED2 | 0.40 | 7.41E−05 | −1.20E−16 | 1.00E+00 | 3.89E−04 | 0.40 | 2.18E−02 |
| ARID3A | 0.76 | 2.26E−03 | 7.98E−01 | 1.15E−02 | 3.89E−04 | 0.89 | 2.18E−02 |
| GAK | 0.56 | 7.57E−05 | −1.20E−16 | 1.00E+00 | 3.97E−04 | 0.56 | 2.20E−02 |
| TTC39C | 0.86 | 1.60E−02 | 1.02E+00 | 1.69E−03 | 3.96E−04 | 1.62 | 2.20E−02 |
| VPS37A | 0.74 | 3.73E−03 | 1.04E+00 | 7.06E−03 | 3.96E−04 | 1.19 | 2.20E−02 |
| DSTN | 0.71 | 6.43E−04 | 9.26E−01 | 4.52E−02 | 3.98E−04 | 1.34 | 2.20E−02 |
| BEND4 | 0.56 | 2.03E−02 | 1.11E+00 | 1.38E−03 | 4.07E−04 | 1.08 | 2.23E−02 |
| CD3E | 0.57 | 7.91E−05 | −1.20E−16 | 1.00E+00 | 4.14E−04 | 0.57 | 2.26E−02 |
| UPF3A | 0.69 | 2.62E−02 | 1.05E+00 | 1.11E−03 | 4.13E−04 | 1.31 | 2.26E−02 |
| MGAT2 | 0.72 | 5.77E−03 | 1.00E+00 | 4.87E−03 | 4.20E−04 | 1.46 | 2.28E−02 |
| LGALS1 | 0.42 | 8.25E−05 | −1.20E−16 | 1.00E+00 | 4.31E−04 | 0.42 | 2.33E−02 |
| GATM | 0.39 | 1.48E−01 | 1.49E+00 | 2.53E−04 | 4.34E−04 | 1.80 | 2.34E−02 |
| MDFIC | 0.78 | 5.18E−04 | 6.89E−01 | 6.54E−02 | 4.43E−04 | 1.07 | 2.37E−02 |
| RPL31−PS12 | −0.02 | 9.34E−01 | −1.30E+00 | 8.68E−05 | 4.50E−04 | −1.82 | 2.40E−02 |
| H2−Q7 | −0.60 | 3.03E−03 | −2.58E+00 | 1.02E−02 | 4.54E−04 | −0.93 | 2.41E−02 |
| RNF115 | 0.87 | 5.29E−03 | 9.11E−01 | 5.88E−03 | 4.61E−04 | 1.49 | 2.44E−02 |
| FAM174A | 0.17 | 4.67E−01 | 1.27E+00 | 1.20E−04 | 4.70E−04 | 1.39 | 2.48E−02 |
| CTBP1 | 0.91 | 1.57E−04 | 3.53E−01 | 3.17E−01 | 4.80E−04 | 0.97 | 2.52E−02 |
| ZBTB38 | 0.64 | 4.65E−02 | 1.10E+00 | 7.69E−04 | 4.81E−04 | 1.28 | 2.52E−02 |
| ZFP973 | 0.41 | 6.58E−02 | −1.17E+00 | 5.67E−04 | 4.84E−04 | −0.38 | 2.52E−02 |
| 1810026J23RIK | 0.02 | 9.45E−01 | 1.31E+00 | 9.47E−05 | 4.90E−04 | 1.26 | 2.54E−02 |
| TDP2 | 0.72 | 1.50E−02 | 1.00E+00 | 2.31E−03 | 5.00E−04 | 1.46 | 2.58E−02 |
| RPL39 | −0.20 | 9.89E−05 | −1.20E−16 | 1.00E+00 | 5.11E−04 | −0.20 | 2.63E−02 |
| RAI14 | −0.43 | 3.50E−01 | 1.43E+00 | 1.60E−04 | 5.21E−04 | 0.60 | 2.67E−02 |
| ADAMTSL4 | −0.60 | 1.52E−01 | −1.20E+00 | 3.06E−04 | 5.30E−04 | −0.91 | 2.71E−02 |
| ZFAND6 | 0.66 | 1.07E−03 | 1.41E+00 | 3.66E−02 | 5.35E−04 | 1.05 | 2.71E−02 |
| MCUR1 | 0.46 | 1.15E−01 | 1.15E+00 | 3.88E−04 | 5.35E−04 | 0.95 | 2.71E−02 |
| MYLK | 0.16 | 8.03E−01 | 1.92E+00 | 1.08E−04 | 5.39E−04 | 0.38 | 2.72E−02 |
| ANKRD13C | 1.07 | 1.97E−03 | 7.22E−01 | 1.98E−02 | 5.52E−04 | 1.05 | 2.75E−02 |
| IARS | −0.79 | 1.11E−04 | 3.38E−01 | 7.91E−01 | 5.50E−04 | −0.70 | 2.75E−02 |
| GBP10 | −0.45 | 2.53E−01 | −1.18E+00 | 2.15E−04 | 5.52E−04 | −1.49 | 2.75E−02 |
| PSMD3 | 0.55 | 1.10E−04 | −6.33E−02 | 9.04E−01 | 5.59E−04 | 0.55 | 2.78E−02 |
| MIR684−2 | −1.96 | 5.81E−02 | −1.59E+00 | 7.60E−04 | 5.73E−04 | −1.46 | 2.84E−02 |
| ACLY | −0.55 | 1.12E−04 | −1.20E−16 | 1.00E+00 | 5.75E−04 | −0.55 | 2.84E−02 |
| CRIP2 | −0.25 | 5.85E−01 | 1.46E+00 | 1.36E−04 | 5.96E−04 | 0.98 | 2.93E−02 |
| FRMD6 | −0.61 | 3.62E−02 | 1.09E+00 | 1.23E−03 | 6.00E−04 | 0.62 | 2.93E−02 |
| ST3GAL1 | 0.83 | 1.79E−03 | 8.24E−01 | 2.45E−02 | 6.09E−04 | 1.22 | 2.97E−02 |
| LYSMD2 | 1.26 | 1.44E−04 | 1.87E−01 | 5.53E−01 | 6.12E−04 | 0.72 | 2.97E−02 |
| PANK4 | −0.96 | 3.79E−04 | −4.76E−01 | 1.48E−01 | 6.35E−04 | −0.96 | 3.06E−02 |
| CACNB2 | 1.46 | 6.17E−03 | 1.12E+00 | 7.16E−03 | 6.33E−04 | 0.41 | 3.06E−02 |
| CDCA7 | −0.42 | 7.05E−02 | −1.81E+00 | 7.24E−04 | 6.43E−04 | −1.16 | 3.09E−02 |
| CRISPLD2 | 0.34 | 6.24E−01 | 1.64E+00 | 1.45E−04 | 6.49E−04 | 0.69 | 3.10E−02 |
| GM4951 | −1.08 | 1.21E−01 | −1.37E+00 | 4.67E−04 | 6.59E−04 | −0.95 | 3.14E−02 |
| CHST10 | 2.47 | 5.56E−03 | 1.45E+00 | 8.48E−03 | 6.70E−04 | 0.52 | 3.18E−02 |
| IFT57 | 0.71 | 1.37E−02 | 9.23E−01 | 3.52E−03 | 6.76E−04 | 1.23 | 3.19E−02 |
| PCBP3 | −1.85 | 1.12E−03 | −1.09E+00 | 4.59E−02 | 6.74E−04 | −0.27 | 3.19E−02 |
| GGA3 | 0.54 | 8.38E−02 | −1.18E+00 | 6.74E−04 | 6.93E−04 | −0.37 | 3.24E−02 |
| TESC | 0.79 | 1.22E−02 | 9.32E−01 | 4.02E−03 | 6.90E−04 | 1.54 | 3.24E−02 |
| EBF1 | 0.47 | 5.34E−02 | 1.35E+00 | 1.01E−03 | 6.94E−04 | 1.37 | 3.24E−02 |
| CTSD | 0.63 | 5.01E−04 | 1.87E+00 | 1.22E−01 | 7.10E−04 | 0.73 | 3.30E−02 |
| ETNK1 | −0.62 | 2.11E−03 | −8.95E−01 | 2.54E−02 | 7.28E−04 | −1.00 | 3.37E−02 |
| CBLN3 | 0.43 | 5.60E−03 | −8.11E−01 | 9.30E−03 | 7.32E−04 | 0.04 | 3.38E−02 |
| NHLRC2 | 0.32 | 3.47E−01 | 1.16E+00 | 2.34E−04 | 7.38E−04 | 1.14 | 3.39E−02 |
| S100A11 | 0.76 | 1.44E−04 | 2.05E−01 | 1.00E+00 | 7.40E−04 | 0.78 | 3.39E−02 |
| DHX57 | −1.00 | 2.05E−04 | −2.35E−01 | 4.44E−01 | 7.57E−04 | −0.59 | 3.46E−02 |
| E030024N20RIK | −0.38 | 1.52E−04 | −1.20E−16 | 1.00E+00 | 7.67E−04 | −0.38 | 3.49E−02 |
| NLRC5 | −0.32 | 1.75E−01 | −1.85E+00 | 4.08E−04 | 7.71E−04 | −0.87 | 3.49E−02 |
| DTX3L | −0.74 | 1.74E−04 | −3.67E−01 | 6.39E−01 | 7.79E−04 | −0.76 | 3.52E−02 |
| EIF4G2 | −0.55 | 1.55E−04 | −1.20E−16 | 1.00E+00 | 7.83E−04 | −0.55 | 3.52E−02 |
| SLC25A39 | 0.53 | 1.74E−04 | −2.55E−01 | 6.76E−01 | 7.99E−04 | 0.45 | 3.53E−02 |
| ISCU | 0.57 | 4.38E−03 | 1.33E+00 | 1.31E−02 | 7.92E−04 | 1.37 | 3.53E−02 |
| BACH2 | 0.73 | 6.50E−04 | 9.31E−01 | 1.04E−01 | 7.95E−04 | 0.90 | 3.53E−02 |
| MGLL | 0.44 | 6.29E−02 | 1.26E+00 | 1.01E−03 | 7.98E−04 | 1.48 | 3.53E−02 |
| PTBP2 | −1.13 | 7.27E−04 | −5.25E−01 | 9.03E−02 | 7.90E−04 | −0.85 | 3.53E−02 |
| APLF | 0.37 | 1.63E−01 | −1.33E+00 | 4.54E−04 | 8.08E−04 | −0.27 | 3.56E−02 |
| ANXA3 | 0.60 | 7.99E−02 | −1.14E+00 | 8.46E−04 | 8.23E−04 | −1.13 | 3.61E−02 |
| TNFRSF4 | −0.45 | 1.63E−04 | −1.20E−16 | 1.00E+00 | 8.20E−04 | −0.45 | 3.61E−02 |
| MXD4 | 3.36 | 9.34E−03 | 1.96E+00 | 6.44E−03 | 8.34E−04 | 0.29 | 3.64E−02 |
| ARHGDIB | −0.32 | 1.68E−04 | −1.20E−16 | 1.00E+00 | 8.41E−04 | −0.32 | 3.66E−02 |
| HIST1H1E | 0.70 | 3.16E−03 | 8.23E−01 | 1.97E−02 | 8.46E−04 | 1.45 | 3.67E−02 |
| DCTN4 | 0.65 | 5.94E−04 | 1.88E+00 | 1.26E−01 | 8.54E−04 | 0.73 | 3.68E−02 |
| KIF3A | −0.48 | 3.43E−02 | −1.13E+00 | 1.90E−03 | 8.56E−04 | −1.23 | 3.68E−02 |
| GPM6B | −0.78 | 3.29E−02 | −9.69E−01 | 1.97E−03 | 8.57E−04 | −1.67 | 3.68E−02 |
| DUSP3 | 0.69 | 3.01E−02 | 1.02E+00 | 2.16E−03 | 8.64E−04 | 1.27 | 3.69E−02 |
| RPL9 | −0.18 | 1.73E−04 | −1.20E−16 | 1.00E+00 | 8.67E−04 | −0.18 | 3.70E−02 |
| SEMA7A | 0.59 | 1.32E−01 | 1.12E+00 | 5.84E−04 | 8.71E−04 | 1.19 | 3.70E−02 |
| INPP4B | −0.16 | 5.50E−01 | −1.67E+00 | 2.14E−04 | 8.86E−04 | −1.02 | 3.75E−02 |
| GNAI2 | 0.56 | 2.24E−04 | −5.96E−01 | 5.22E−01 | 8.99E−04 | 0.53 | 3.79E−02 |
| TRIM25 | −0.37 | 5.86E−02 | −2.23E+00 | 1.26E−03 | 9.21E−04 | −0.70 | 3.87E−02 |
| PRG4 | 0.53 | 1.59E−01 | −1.44E+00 | 5.39E−04 | 9.31E−04 | −0.18 | 3.91E−02 |
| H1F0 | 0.97 | 9.21E−04 | 5.78E−01 | 8.52E−02 | 9.38E−04 | 1.19 | 3.92E−02 |
| PKNOX1 | −0.68 | 4.56E−02 | 9.93E−01 | 1.63E−03 | 9.50E−04 | 0.58 | 3.95E−02 |
| TRIP10 | −0.86 | 2.25E−04 | 1.95E−01 | 5.78E−01 | 9.51E−04 | −0.37 | 3.95E−02 |
| RNF10 | 0.82 | 2.43E−04 | 3.56E−01 | 5.38E−01 | 9.84E−04 | 0.88 | 3.99E−02 |
| HMGB1 | −0.45 | 1.98E−04 | −1.20E−16 | 1.00E+00 | 9.85E−04 | −0.45 | 3.99E−02 |
| LMNA | 0.52 | 1.63E−01 | 1.13E+00 | 5.57E−04 | 9.80E−04 | 1.75 | 3.99E−02 |
| SOAT2 | −0.63 | 2.91E−02 | −1.26E+00 | 2.56E−03 | 9.80E−04 | −0.94 | 3.99E−02 |
| HMGN2 | −0.73 | 2.35E−03 | −1.27E+00 | 3.17E−02 | 9.72E−04 | −0.99 | 3.99E−02 |
| GM20939 | −0.57 | 2.07E−02 | −1.52E+00 | 3.51E−03 | 9.70E−04 | −0.93 | 3.99E−02 |
| MAX | 0.69 | 2.32E−04 | −3.12E−01 | 5.79E−01 | 9.79E−04 | 0.56 | 3.99E−02 |
| CEBPD | 2.35 | 2.74E−02 | 1.87E+00 | 2.68E−03 | 9.68E−04 | 0.34 | 3.99E−02 |
| ARHGAP18 | −0.33 | 1.43E−01 | −1.14E+00 | 6.32E−04 | 9.93E−04 | −1.36 | 4.01E−02 |
| THY1 | 0.58 | 2.06E−04 | −1.20E−16 | 1.00E+00 | 1.02E−03 | 0.57 | 4.10E−02 |
| CNN3 | 0.31 | 3.79E−02 | 2.85E+00 | 2.12E−03 | 1.03E−03 | 0.87 | 4.14E−02 |
| CPNE8 | −0.36 | 4.91E−02 | −1.29E+00 | 1.68E−03 | 1.04E−03 | −0.96 | 4.16E−02 |
| HMGCR | −0.83 | 2.38E−04 | −3.12E−01 | 6.48E−01 | 1.05E−03 | −0.86 | 4.18E−02 |
| ETS1 | −0.74 | 2.13E−04 | −1.20E−16 | 1.00E+00 | 1.05E−03 | −0.74 | 4.18E−02 |
| MRPL23 | −0.50 | 4.25E−04 | 1.07E+00 | 2.54E−01 | 1.05E−03 | −0.42 | 4.18E−02 |
| TEFM | −1.01 | 5.57E−04 | −4.25E−01 | 1.83E−01 | 1.06E−03 | −0.79 | 4.21E−02 |
| RPLP1 | −0.19 | 2.20E−04 | −1.20E−16 | 1.00E+00 | 1.08E−03 | −0.19 | 4.27E−02 |
| WASF2 | 0.52 | 2.22E−04 | −1.20E−16 | 1.00E+00 | 1.09E−03 | 0.52 | 4.29E−02 |
| NME2 | 0.34 | 2.70E−04 | 9.82E−01 | 5.46E−01 | 1.10E−03 | 0.36 | 4.30E−02 |
| UBXN2A | 1.06 | 2.55E−04 | 1.70E−01 | 6.21E−01 | 1.10E−03 | 0.87 | 4.30E−02 |
| MYO10 | 0.62 | 7.30E−03 | 1.14E+00 | 1.19E−02 | 1.15E−03 | 0.93 | 4.49E−02 |
| GNPDA2 | 0.68 | 4.91E−03 | 8.88E−01 | 1.79E−02 | 1.16E−03 | 1.50 | 4.49E−02 |
| PHF2 | 0.35 | 7.46E−02 | −1.01E+00 | 1.30E−03 | 1.16E−03 | −0.22 | 4.49E−02 |
| KDSR | 1.07 | 2.77E−04 | −2.18E−01 | 5.99E−01 | 1.17E−03 | 0.77 | 4.50E−02 |
| RFX3 | 1.39 | 2.26E−02 | −1.18E+00 | 3.96E−03 | 1.17E−03 | −0.10 | 4.50E−02 |
| ILTIFB | | 1.00E+00 | 2.89E+00 | 1.17E−03 | 1.17E−03 | | 4.50E−02 |
| PNO1 | 0.60 | 3.60E−03 | 1.59E+00 | 2.54E−02 | 1.18E−03 | 1.13 | 4.53E−02 |
| TIAL1 | −0.40 | 1.33E−01 | −1.10E+00 | 8.10E−04 | 1.19E−03 | −0.82 | 4.53E−02 |
| MYC | 0.79 | 3.07E−04 | −3.76E−01 | 5.24E−01 | 1.21E−03 | 0.61 | 4.61E−02 |
| RNF214 | −0.85 | 2.53E−04 | 4.19E−02 | 9.24E−01 | 1.23E−03 | −0.53 | 4.66E−02 |
| SLC25A33 | 1.62 | 7.18E−04 | 5.45E−01 | 1.66E−01 | 1.26E−03 | 0.55 | 4.75E−02 |
| DCLK1 | 0.71 | 1.81E−02 | 9.19E−01 | 5.40E−03 | 1.28E−03 | 1.08 | 4.82E−02 |
| FUNDC2 | −0.46 | 3.04E−02 | −1.03E+00 | 3.31E−03 | 1.29E−03 | −1.13 | 4.83E−02 |
| FAU | −0.19 | 2.68E−04 | −1.20E−16 | 1.00E+00 | 1.31E−03 | −0.19 | 4.89E−02 |
| RAB24 | −0.09 | 7.96E−01 | 1.21E+00 | 2.79E−04 | 1.31E−03 | 1.01 | 4.89E−02 |
| DGAT2 | 0.90 | 4.85E−01 | −2.43E+00 | 3.50E−04 | 1.32E−03 | −0.41 | 4.90E−02 |
| UBE2D3 | 0.36 | 5.33E−04 | 1.43E+00 | 2.68E−01 | 1.34E−03 | 0.40 | 4.99E−02 |
| SRSF11 | 0.59 | 5.36E−04 | 1.43E+00 | 2.68E−01 | 1.35E−03 | 0.63 | 5.00E−02 |
| SYVN1 | 0.62 | 5.18E−03 | 9.73E−01 | 2.04E−02 | 1.37E−03 | 0.98 | 5.04E−02 |
| H2-T22 | −0.61 | 2.81E−04 | −1.20E−16 | 1.00E+00 | 1.36E−03 | −0.61 | 5.04E−02 |
| IL18BP | −1.87 | 3.37E−04 | −2.30E−01 | 5.69E−01 | 1.38E−03 | −0.45 | 5.05E−02 |
| IFT46 | 0.93 | 8.85E−04 | 5.26E−01 | 1.47E−01 | 1.39E−03 | 1.04 | 5.08E−02 |
| ADAM10 | −0.64 | 1.99E−03 | −9.08E−01 | 5.96E−02 | 1.43E−03 | −0.97 | 5.21E−02 |
| SLC43A1 | 1.42 | 2.36E−01 | −2.36E+00 | 6.39E−04 | 1.45E−03 | −0.32 | 5.30E−02 |
| DDB2 | −1.01 | 4.65E−03 | −7.13E−01 | 2.48E−02 | 1.47E−03 | −1.26 | 5.34E−02 |
| H2-Q9 | −0.71 | 2.65E−02 | −9.25E−01 | 4.49E−03 | 1.50E−03 | −1.68 | 5.44E−02 |
| SSBP4 | 0.67 | 1.01E−02 | 8.65E−01 | 1.17E−02 | 1.52E−03 | 1.36 | 5.50E−02 |
| ULBP1 | 0.16 | 7.02E−01 | 1.27E+00 | 3.48E−04 | 1.55E−03 | 1.14 | 5.56E−02 |
| MICU2 | 0.83 | 7.01E−04 | 4.76E−01 | 2.27E−01 | 1.55E−03 | 1.10 | 5.56E−02 |
| GFPT2 | 0.57 | 1.79E−01 | 1.23E+00 | 8.55E−04 | 1.56E−03 | 0.92 | 5.60E−02 |
| ROGDI | 0.19 | 6.48E−01 | −1.19E+00 | 3.65E−04 | 1.57E−03 | −1.12 | 5.62E−02 |
| HIST2H3B | 1.06 | 5.77E−04 | 3.80E−01 | 3.05E−01 | 1.58E−03 | 0.62 | 5.62E−02 |
| IRGM1 | −0.48 | 7.39E−02 | −1.47E+00 | 1.86E−03 | 1.60E−03 | −1.20 | 5.64E−02 |
| GLTSCR2 | −0.29 | 3.34E−04 | −1.20E−16 | 1.00E+00 | 1.61E−03 | −0.29 | 5.64E−02 |
| CCR8 | −0.78 | 6.94E−03 | −8.18E−01 | 1.81E−02 | 1.60E−03 | −1.57 | 5.64E−02 |
| IL16 | −0.60 | 2.13E−02 | −9.43E−01 | 5.94E−03 | 1.61E−03 | −1.27 | 5.64E−02 |
| APOPT1 | −0.81 | 1.00E−03 | −6.98E−01 | 1.52E−01 | 1.60E−03 | −1.07 | 5.64E−02 |
| FLOT1 | 0.58 | 4.20E−03 | 1.32E+00 | 3.10E−02 | 1.62E−03 | 1.13 | 5.65E−02 |
| ESYT1 | 0.57 | 3.98E−03 | 1.47E+00 | 3.26E−02 | 1.61E−03 | 0.95 | 5.65E−02 |
| ATP6V1H | −0.50 | 3.31E−02 | −9.79E−01 | 3.94E−03 | 1.62E−03 | −1.50 | 5.65E−02 |
| SEMA4F | 0.53 | 9.72E−02 | 1.05E+00 | 1.50E−03 | 1.64E−03 | 1.52 | 5.69E−02 |
| TMEM50A | 0.38 | 3.42E−04 | −1.20E−16 | 1.00E+00 | 1.64E−03 | 0.38 | 5.69E−02 |
| AUP1 | −0.41 | 3.45E−04 | −1.20E−16 | 1.00E+00 | 1.65E−03 | −0.41 | 5.72E−02 |
| ADAM28 | 0.41 | 4.34E−03 | 6.73E−01 | 3.13E−02 | 1.68E−03 | 0.34 | 5.80E−02 |

TABLE 1B
Th1 KO GFP+ vs GFP−

| Gene | coefC | pvalC | coefD | pvalD | pvalH | logFC | FDR |
|---|---|---|---|---|---|---|---|
| IL23R | 1.17 | 1.84E−02 | 3.63E+00 | 3.38E−20 | 2.45E−20 | 3.97 | 3.17E−16 |
| EGFP_IRES_IL23R | 0.75 | 2.22E−01 | 2.77E+00 | 5.60E−13 | 2.44E−12 | 3.48 | 1.58E−08 |
| GZMB | −1.78 | 1.06E−03 | −1.69E+00 | 1.27E−06 | 3.77E−08 | −3.93 | 1.50E−04 |
| CHCHD10 | −1.17 | 1.68E−08 | −1.31E+00 | 1.64E−01 | 4.64E−08 | −1.33 | 1.50E−04 |
| ARPC3 | 0.96 | 1.19E−07 | 1.73E+00 | 2.91E−02 | 7.56E−08 | 1.32 | 1.96E−04 |
| TPT1 | −0.51 | 2.53E−08 | −3.03E−17 | 1.00E+00 | 1.82E−07 | −0.51 | 3.94E−04 |
| TIMP1 | 0.80 | 1.14E−01 | 1.86E+00 | 1.08E−07 | 2.13E−07 | 3.64 | 3.94E−04 |
| FAM162A | 0.93 | 1.25E−07 | 1.04E+00 | 4.91E−01 | 6.77E−07 | 0.95 | 1.10E−03 |
| SMOX | 1.10 | 7.85E−06 | 2.52E+00 | 1.22E−02 | 1.99E−06 | 1.37 | 2.87E−03 |
| GM8096 | | 1.00E+00 | −3.73E+00 | 3.81E−06 | 3.81E−06 | | 4.49E−03 |
| ANP32B | −1.04 | 8.44E−06 | −1.55E+00 | 2.26E−02 | 3.67E−06 | −1.17 | 4.49E−03 |
| S100A10 | 0.73 | 9.67E−07 | −3.03E−17 | 1.00E+00 | 6.17E−06 | 0.73 | 6.66E−03 |
| PHF11C | −1.84 | 9.44E−05 | −1.16E+00 | 3.84E−03 | 7.49E−06 | −1.16 | 7.42E−03 |
| IRF7 | −0.62 | 4.18E−02 | −1.51E+00 | 1.10E−05 | 8.01E−06 | −2.17 | 7.42E−03 |
| SPHK1 | −1.19 | 2.91E−01 | 2.67E+00 | 2.71E−06 | 9.50E−06 | 1.22 | 8.06E−03 |
| TNC | −0.49 | 5.40E−02 | 1.66E+00 | 1.10E−05 | 9.94E−06 | 0.29 | 8.06E−03 |
| RPS6KC1 | −1.07 | 6.49E−02 | 1.69E+00 | 1.06E−05 | 1.12E−05 | 0.97 | 8.52E−03 |
| PRDX2 | 0.65 | 2.69E−06 | −3.03E−17 | 1.00E+00 | 1.65E−05 | 0.65 | 9.37E−03 |
| TNFRSF8 | 0.75 | 2.12E−02 | 1.60E+00 | 4.38E−05 | 1.66E−05 | 1.92 | 9.37E−03 |
| EBF1 | 0.34 | 3.16E−01 | 1.66E+00 | 4.39E−06 | 1.60E−05 | 1.57 | 9.37E−03 |
| BHLHE40 | 0.99 | 4.08E−06 | 1.45E+00 | 3.30E−01 | 1.53E−05 | 1.02 | 9.37E−03 |
| ATP6V0D2 | 1.48 | 2.44E−03 | 1.24E+00 | 3.18E−04 | 1.55E−05 | 2.42 | 9.37E−03 |
| H2−Q10 | 0.99 | 9.72E−03 | 1.53E+00 | 8.35E−05 | 1.54E−05 | 2.35 | 9.37E−03 |
| INSL5 | 3.64 | 3.57E−03 | 2.52E+00 | 2.57E−04 | 1.80E−05 | 0.92 | 9.72E−03 |
| IFITM2 | 0.84 | 2.15E−05 | 1.48E+00 | 7.92E−02 | 2.58E−05 | 1.12 | 1.34E−02 |
| SERPINF1 | 1.36 | 3.38E−05 | 8.46E−01 | 7.16E−02 | 3.65E−05 | 1.79 | 1.82E−02 |
| S1PR1 | 1.10 | 9.87E−05 | 1.25E+00 | 2.36E−02 | 3.93E−05 | 1.44 | 1.82E−02 |
| P4HA2 | 1.14 | 3.09E−03 | 1.18E+00 | 7.20E−04 | 4.12E−05 | 1.96 | 1.82E−02 |
| ASS1 | −0.95 | 1.11E−04 | −1.05E+00 | 2.02E−02 | 3.84E−05 | −1.61 | 1.82E−02 |
| AP1B1 | 0.39 | 1.56E−01 | 2.70E+00 | 2.05E−05 | 4.21E−05 | 1.19 | 1.82E−02 |
| PDCD1 | 0.63 | 6.70E−02 | 1.52E+00 | 4.38E−05 | 4.41E−05 | 2.17 | 1.82E−02 |
| RPSA | −0.31 | 7.66E−06 | −3.03E−17 | 1.00E+00 | 4.49E−05 | −0.31 | 1.82E−02 |
| AW554918 | −2.25 | 1.02E−05 | 8.16E−02 | 8.82E−01 | 5.86E−05 | −0.37 | 2.23E−02 |
| KCTD11 | 0.08 | 9.12E−01 | 1.84E+00 | 1.01E−05 | 5.79E−05 | 1.24 | 2.23E−02 |
| MGARP | 0.44 | 9.71E−02 | 1.70E+00 | 4.97E−05 | 6.73E−05 | 2.03 | 2.42E−02 |
| AHNAK | 1.34 | 2.86E−05 | 1.21E+00 | 1.86E−01 | 6.58E−05 | 1.40 | 2.42E−02 |
| ITFG2 | −1.36 | 1.07E−04 | −7.36E−01 | 4.28E−02 | 7.06E−05 | −0.97 | 2.47E−02 |
| SLC7A14 | −0.44 | 1.89E−04 | −1.90E+00 | 2.43E−02 | 7.44E−05 | −0.48 | 2.54E−02 |
| CD200 | 1.05 | 4.03E−04 | 2.51E+00 | 1.13E−02 | 7.74E−05 | 1.24 | 2.57E−02 |
| NCF4 | −0.45 | 1.09E−01 | −1.62E+00 | 5.74E−05 | 8.44E−05 | −1.74 | 2.74E−02 |
| GARS | −0.71 | 5.86E−05 | −1.37E+00 | 1.34E−01 | 1.01E−04 | −0.75 | 2.87E−02 |
| LAMB3 | −1.61 | 1.67E−01 | −2.77E+00 | 5.05E−05 | 1.04E−04 | −0.48 | 2.87E−02 |
| VHL | −0.32 | 3.76E−01 | 1.44E+00 | 2.77E−05 | 1.03E−04 | 1.37 | 2.87E−02 |
| CCDC71L | 0.43 | 4.05E−01 | 1.61E+00 | 2.58E−05 | 1.01E−04 | 1.12 | 2.87E−02 |
| ARHGEF1 | −0.82 | 1.82E−05 | 3.31E−01 | 7.33E−01 | 9.67E−05 | −0.75 | 2.87E−02 |
| AMDHD1 | | 1.00E+00 | −3.43E+00 | 9.86E−05 | 9.86E−05 | | 2.87E−02 |
| LUM | 1.22 | 5.18E−01 | 2.17E+00 | 2.18E−05 | 9.89E−05 | 1.56 | 2.87E−02 |
| CCDC102A | | 1.00E+00 | 3.16E+00 | 1.22E−04 | 1.22E−04 | | 3.24E−02 |
| DOCK8 | −1.05 | 4.53E−05 | 4.39E−01 | 2.46E−01 | 1.25E−04 | −0.52 | 3.24E−02 |
| RC3H1 | −0.91 | 4.24E−04 | −8.27E−01 | 1.81E−02 | 1.22E−04 | −1.02 | 3.24E−02 |
| HVCN1 | 0.52 | 6.37E−02 | 1.43E+00 | 1.57E−04 | 1.42E−04 | 1.88 | 3.60E−02 |
| RABGAP1 | 1.46 | 5.17E−04 | 7.96E−01 | 1.98E−02 | 1.60E−04 | 1.14 | 3.98E−02 |
| RORA | 0.14 | 6.69E−01 | 1.46E+00 | 3.27E−05 | 1.64E−04 | 1.07 | 4.01E−02 |
| RAC1 | −1.00 | 1.08E−04 | −1.94E+00 | 1.26E−01 | 1.72E−04 | −1.03 | 4.05E−02 |
| IZUMO1R | −0.89 | 2.76E−03 | −1.02E+00 | 3.72E−03 | 1.69E−04 | −1.97 | 4.05E−02 |
| SPTAN1 | −1.12 | 4.17E−05 | 2.41E−01 | 5.78E−01 | 1.93E−04 | −0.77 | 4.41E−02 |
| CD44 | 0.18 | 5.40E−01 | 1.82E+00 | 4.34E−05 | 1.94E−04 | 1.36 | 4.41E−02 |
| RNH1 | 0.67 | 4.22E−05 | 8.23E−01 | 7.11E−01 | 2.13E−04 | 0.68 | 4.74E−02 |
| MPHOSPH6 | 0.84 | 2.64E−04 | −1.07E+00 | 5.88E−02 | 2.16E−04 | 0.63 | 4.74E−02 |
| SAA4 | 0.15 | 4.53E−01 | −1.68E+00 | 5.84E−05 | 2.34E−04 | −0.20 | 4.97E−02 |
| SLC15A3 | 0.98 | 2.93E−02 | 1.24E+00 | 5.32E−04 | 2.31E−04 | 2.03 | 4.97E−02 |
| AZIN2 | −2.18 | 6.54E−05 | 4.58E−01 | 3.86E−01 | 2.38E−04 | −0.12 | 4.97E−02 |
| GINM1 | 0.08 | 6.96E−01 | 1.81E+00 | 4.94E−05 | 2.45E−04 | 1.43 | 5.05E−02 |
| SERPINA3F | −3.97 | 5.18E−04 | −1.37E+00 | 3.34E−02 | 2.52E−04 | −0.69 | 5.06E−02 |
| MIR703 | 0.17 | 2.73E−01 | −1.55E+00 | 8.91E−05 | 2.54E−04 | −2.61 | 5.06E−02 |
| TESC | −0.89 | 1.40E−03 | −8.94E−01 | 1.27E−02 | 2.72E−04 | −1.70 | 5.16E−02 |
| IER2 | −0.98 | 8.39E−05 | −5.54E−01 | 3.21E−01 | 2.67E−04 | −1.06 | 5.16E−02 |
| HMGN2 | −0.63 | 1.38E−02 | −1.49E+00 | 1.32E−03 | 2.79E−04 | −1.08 | 5.16E−02 |
| RNASEK | 0.27 | 2.40E−01 | 3.28E+00 | 1.02E−04 | 2.64E−04 | 1.09 | 5.16E−02 |
| SLC7A5 | −0.93 | 3.83E−03 | −1.08E+00 | 4.64E−03 | 2.78E−04 | −1.24 | 5.16E−02 |
| PGAM1 | 0.66 | 5.65E−05 | 5.86E−01 | 8.08E−01 | 2.92E−04 | 0.67 | 5.19E−02 |
| MTF2 | −0.31 | 3.26E−01 | −1.57E+00 | 9.07E−05 | 2.91E−04 | −1.24 | 5.19E−02 |
| YKT6 | −0.73 | 1.43E−03 | 1.07E+00 | 1.32E−02 | 2.87E−04 | −0.06 | 5.19E−02 |
| INPP5B | 0.94 | 8.05E−05 | 4.90E−01 | 4.27E−01 | 3.07E−04 | 1.03 | 5.29E−02 |
| ARF6 | −0.95 | 2.72E−04 | −8.36E−01 | 8.95E−02 | 3.13E−04 | −1.05 | 5.29E−02 |
| CDT1 | −0.93 | 5.72E−04 | −8.47E−01 | 3.73E−02 | 3.03E−04 | −1.52 | 5.29E−02 |
| TRIM24 | −1.17 | 3.45E−03 | −9.70E−01 | 5.92E−03 | 3.14E−04 | −0.96 | 5.29E−02 |
| CAMK2B | | 1.00E+00 | 3.62E+00 | 3.37E−04 | 3.37E−04 | | 5.60E−02 |
| ZC3H12C | −1.13 | 1.68E−01 | 2.20E+00 | 1.77E−04 | 3.42E−04 | 0.41 | 5.61E−02 |
| GABARAPL1 | 0.41 | 9.16E−02 | 3.08E+00 | 3.13E−04 | 3.64E−04 | 0.99 | 5.77E−02 |
| BNIP3 | 0.32 | 2.23E−01 | 2.05E+00 | 1.48E−04 | 3.57E−04 | 1.66 | 5.77E−02 |
| TMBIM4 | 0.06 | 7.87E−01 | 3.26E+00 | 7.20E−05 | 3.65E−04 | 1.01 | 5.77E−02 |
| ETV5 | 0.23 | 5.98E−01 | 1.39E+00 | 8.49E−05 | 3.85E−04 | 1.61 | 6.01E−02 |
| SERF1 | 0.86 | 4.65E−04 | 6.83E−01 | 6.59E−02 | 4.03E−04 | 1.39 | 6.07E−02 |
| TRAF3IP3 | −0.43 | 9.12E−02 | −2.00E+00 | 3.47E−04 | 3.99E−04 | −1.18 | 6.07E−02 |
| KHDRBS1 | −0.88 | 2.32E−04 | −7.20E−01 | 1.46E−01 | 3.96E−04 | −1.01 | 6.07E−02 |
| GDF3 | | 1.00E+00 | −3.20E+00 | 4.08E−04 | 4.08E−04 | | 6.08E−02 |
| SLC13A3 | 0.92 | 3.81E−01 | 1.99E+00 | 1.19E−04 | 4.16E−04 | 0.78 | 6.13E−02 |
| ISG20 | −0.74 | 1.06E−01 | −1.37E+00 | 3.31E−04 | 4.30E−04 | −1.74 | 6.27E−02 |
| PHPT1 | −0.18 | 5.04E−01 | −1.62E+00 | 1.09E−04 | 4.47E−04 | −1.42 | 6.43E−02 |
| SLC17A9 | 0.77 | 8.71E−03 | 1.48E+00 | 3.63E−03 | 4.66E−04 | 1.29 | 6.64E−02 |
| MT1 | 0.89 | 3.74E−04 | 1.16E+00 | 1.04E−01 | 4.77E−04 | 1.12 | 6.72E−02 |
| SYNGAP1 | −0.49 | 3.12E−01 | 1.46E+00 | 1.60E−04 | 4.83E−04 | 0.48 | 6.74E−02 |
| CSNK1G2 | −0.99 | 4.37E−04 | 5.87E−01 | 9.77E−02 | 5.24E−04 | −0.11 | 7.15E−02 |
| PRMT5 | −0.83 | 1.02E−03 | −9.03E−01 | 3.76E−02 | 5.24E−04 | −1.18 | 7.15E−02 |
| SOX5 | 0.67 | 3.12E−02 | 1.11E+00 | 1.24E−03 | 5.35E−04 | 1.10 | 7.22E−02 |
| CYBRD1 | −0.24 | 3.12E−02 | −1.24E+00 | 1.36E−03 | 5.81E−04 | −0.35 | 7.68E−02 |
| CALM2 | 0.52 | 1.45E−04 | 1.10E+00 | 4.89E−01 | 5.78E−04 | 0.54 | 7.68E−02 |
| TMEM265 | 0.09 | 8.93E−01 | 1.77E+00 | 1.18E−04 | 5.99E−04 | 0.82 | 7.69E−02 |
| GRINA | −0.82 | 1.71E−04 | −4.37E−01 | 3.90E−01 | 5.92E−04 | −0.94 | 7.69E−02 |
| NUDCD1 | 1.23 | 4.20E−04 | −5.67E−01 | 1.21E−01 | 5.97E−04 | 0.53 | 7.69E−02 |
| PKIB | 0.32 | 5.35E−01 | 1.58E+00 | 1.55E−04 | 6.46E−04 | 0.92 | 8.21E−02 |
| ITGB7 | −0.66 | 9.97E−03 | −1.55E+00 | 4.82E−03 | 6.80E−04 | −1.27 | 8.45E−02 |
| EDA2R | | 1.00E+00 | 3.08E+00 | 6.74E−04 | 6.74E−04 | | 8.45E−02 |
| SLFN1 | −0.27 | 3.60E−01 | −1.59E+00 | 2.10E−04 | 6.85E−04 | −1.41 | 8.45E−02 |
| USP20 | −1.25 | 1.89E−03 | 8.15E−01 | 2.71E−02 | 6.95E−04 | 0.33 | 8.46E−02 |
| PERP | 0.75 | 1.25E−02 | 1.09E+00 | 4.00E−03 | 7.05E−04 | 1.81 | 8.46E−02 |
| SLC4A7 | 0.97 | 7.26E−04 | 8.29E−01 | 7.90E−02 | 7.08E−04 | 1.19 | 8.46E−02 |
| MYLK | 0.61 | 4.30E−01 | 1.70E+00 | 1.96E−04 | 7.11E−04 | 0.58 | 8.46E−02 |
| GABARAP | 0.62 | 1.44E−04 | 5.59E−01 | 8.83E−01 | 7.20E−04 | 0.62 | 8.48E−02 |
| PADI2 | −0.20 | 4.73E−01 | −1.53E+00 | 1.93E−04 | 7.41E−04 | −0.62 | 8.65E−02 |
| CD74 | 1.23 | 3.42E−03 | 8.19E−01 | 1.60E−02 | 7.60E−04 | 1.64 | 8.80E−02 |
| ZFP420 | | 1.00E+00 | −3.16E+00 | 7.87E−04 | 7.87E−04 | | 8.95E−02 |
| AKR1B8 | 0.31 | 2.95E−01 | 1.28E+00 | 2.78E−04 | 7.81E−04 | 1.89 | 8.95E−02 |
| RPL30 | −0.30 | 1.59E−04 | −3.03E−17 | 1.00E+00 | 8.01E−04 | −0.30 | 9.03E−02 |
| SHMT2 | −0.78 | 3.82E−03 | −1.31E+00 | 1.61E−02 | 8.44E−04 | −0.97 | 9.37E−02 |
| RAD23B | −0.61 | 2.03E−03 | −9.57E−01 | 3.29E−02 | 8.80E−04 | −0.80 | 9.37E−02 |
| NOL7 | −0.70 | 5.69E−04 | −9.39E−01 | 1.39E−01 | 8.81E−04 | −0.81 | 9.37E−02 |
| JAK2 | −0.89 | 3.74E−03 | 8.62E−01 | 1.68E−02 | 8.60E−04 | 0.04 | 9.37E−02 |
| CHD7 | −0.77 | 1.74E−04 | 4.52E−02 | 1.00E+00 | 8.79E−04 | −0.76 | 9.37E−02 |
| AKNA | −0.73 | 6.84E−04 | −1.18E+00 | 1.07E−01 | 8.53E−04 | −0.83 | 9.37E−02 |
| MMP11 | 1.03 | 3.94E−01 | −2.32E+00 | 2.57E−04 | 8.72E−04 | −0.45 | 9.37E−02 |
| LAMP1 | −1.10 | 2.19E−04 | −2.71E−01 | 5.41E−01 | 8.98E−04 | −1.12 | 9.39E−02 |
| TRIB3 | −1.58 | 1.90E−03 | −7.58E−01 | 3.61E−02 | 8.94E−04 | −1.43 | 9.39E−02 |
| SGK1 | 2.86 | 3.53E−02 | −1.63E+00 | 2.06E−03 | 9.45E−04 | −0.30 | 9.73E−02 |
| SPIRE1 | 0.38 | 4.69E−01 | 1.47E+00 | 2.50E−04 | 9.41E−04 | 0.92 | 9.73E−02 |
| CLEC2I | −1.41 | 1.96E−04 | 7.37E−02 | 8.33E−01 | 9.54E−04 | −0.69 | 9.74E−02 |
| ZFP109 | 0.12 | 9.13E−01 | −2.22E+00 | 2.00E−04 | 9.86E−04 | −0.56 | 9.91E−02 |
| MARK4 | −1.43 | 4.30E−04 | 4.72E−01 | 2.28E−01 | 9.82E−04 | −0.07 | 9.91E−02 |

TABLE 1C
Th1 Combined Model

| Gene | coefC | pvalC | coefD | pvalD | pvalH | logFC | FDR |
|---|---|---|---|---|---|---|---|
| RPS2 | 1.32 | 4.71E−18 | 2.12E−16 | 1.00E+00 | 5.17E−17 | 1.32 | 3.66E−14 |
| ANP32B | 2.23 | 1.17E−14 | −4.90E−01 | 6.02E−01 | 1.00E−13 | 2.13 | 3.54E−11 |
| 1110008F13RIK | 2.33 | 3.24E−09 | 1.51E+00 | 4.38E−04 | 5.09E−11 | 3.23 | 1.20E−08 |
| RNF187 | 2.11 | 2.26E−09 | 2.21E+00 | 4.97E−03 | 3.37E−10 | 2.69 | 5.96E−08 |
| H2-T23 | −1.06 | 5.68E−11 | 2.12E−16 | 1.00E+00 | 4.77E−10 | −1.06 | 6.74E−08 |
| LSM4 | 1.53 | 1.33E−10 | 1.47E+00 | 2.88E−01 | 6.26E−10 | 1.59 | 7.37E−08 |
| LAMP1 | 2.08 | 4.71E−09 | 1.06E+00 | 6.44E−02 | 6.42E−09 | 2.38 | 6.48E−07 |
| LARS2 | 0.83 | 9.96E−10 | 2.12E−16 | 1.00E+00 | 7.82E−09 | 0.83 | 6.91E−07 |
| RAC1 | 1.85 | 4.53E−09 | 1.91E+00 | 1.30E−01 | 1.09E−08 | 1.88 | 6.99E−07 |
| IER2 | 1.72 | 1.72E−08 | 1.58E+00 | 2.61E−02 | 1.05E−08 | 2.19 | 6.99E−07 |
| TESC | 1.81 | 4.78E−06 | 1.75E+00 | 6.04E−05 | 9.17E−09 | 2.96 | 6.99E−07 |
| PELI1 | 1.83 | 1.25E−07 | 2.10E+00 | 5.89E−03 | 1.92E−08 | 2.22 | 1.13E−06 |
| VAPA | 1.66 | 1.43E−08 | 1.79E+00 | 1.61E−01 | 3.91E−08 | 1.78 | 2.13E−06 |
| RBMS1 | 1.18 | 9.16E−04 | 2.46E+00 | 5.99E−06 | 1.46E−07 | 2.64 | 6.44E−06 |
| RN7S1 | 0.57 | 3.98E−02 | 2.26E+00 | 1.71E−07 | 1.40E−07 | 2.08 | 6.44E−06 |
| RN7S2 | 0.57 | 3.98E−02 | 2.26E+00 | 1.71E−07 | 1.40E−07 | 2.08 | 6.44E−06 |
| EIF3F | 0.98 | 3.35E−08 | 2.12E−16 | 1.00E+00 | 2.39E−07 | 0.98 | 9.94E−06 |
| PPP4C | 1.29 | 6.66E−07 | 2.10E+00 | 7.73E−02 | 9.05E−07 | 1.47 | 3.55E−05 |
| LRP10 | 1.09 | 6.44E−04 | 3.07E+00 | 8.61E−05 | 1.33E−06 | 1.82 | 4.94E−05 |
| TMEM30A | 1.11 | 1.06E−04 | 3.43E+00 | 7.12E−04 | 1.78E−06 | 1.55 | 5.76E−05 |
| NOL7 | 1.42 | 5.48E−07 | 1.08E+00 | 2.14E−01 | 1.65E−06 | 1.52 | 5.76E−05 |
| CLASP2 | 1.88 | 1.33E−06 | 1.48E+00 | 7.94E−02 | 1.79E−06 | 2.00 | 5.76E−05 |
| TMEM160 | 1.36 | 1.86E−05 | 1.45E+00 | 5.29E−03 | 2.14E−06 | 2.12 | 6.58E−05 |
| 2310036022RIK | 1.66 | 9.17E−06 | 1.06E+00 | 1.19E−02 | 2.25E−06 | 2.09 | 6.63E−05 |
| FAM49B | 1.22 | 6.37E−07 | 5.76E−01 | 4.62E−01 | 3.15E−06 | 1.25 | 8.90E−05 |
| ARF6 | 1.60 | 1.04E−06 | 7.63E−01 | 2.44E−01 | 3.37E−06 | 1.63 | 9.17E−05 |
| UCK2 | 1.47 | 2.84E−06 | 1.59E+00 | 7.81E−02 | 3.68E−06 | 1.69 | 9.63E−05 |
| KHDRBS1 | 1.31 | 3.06E−06 | 1.16E+00 | 7.71E−02 | 3.91E−06 | 1.59 | 9.87E−05 |
| TMEM161B | −2.04 | 1.88E−05 | −1.11E+00 | 1.46E−02 | 5.38E−06 | −0.98 | 1.31E−04 |
| NUBP1 | 1.36 | 2.51E−06 | 7.50E−01 | 1.55E−01 | 5.61E−06 | 1.70 | 1.32E−04 |
| UTP18 | 2.04 | 2.58E−06 | 5.60E−01 | 2.04E−01 | 7.07E−06 | 1.84 | 1.61E−04 |
| ESYT1 | 1.12 | 5.72E−06 | 1.44E+00 | 1.25E−01 | 1.05E−05 | 1.32 | 2.31E−04 |
| ENSA | −1.29 | 2.71E−04 | −1.59E+00 | 1.98E−03 | 1.10E−05 | −1.88 | 2.31E−04 |
| STX6 | 1.27 | 3.60E−04 | 1.78E+00 | 1.50E−03 | 1.11E−05 | 2.12 | 2.31E−04 |
| RALA | 0.68 | 6.00E−03 | 2.51E+00 | 1.16E−04 | 1.37E−05 | 1.68 | 2.76E−04 |
| PRKCH | 1.27 | 1.09E−05 | 1.18E+00 | 9.69E−02 | 1.59E−05 | 1.61 | 3.12E−04 |
| CBX3 | −0.76 | 2.96E−06 | 2.12E−16 | 1.00E+00 | 1.81E−05 | −0.76 | 3.46E−04 |
| NDFIP2 | 0.01 | 9.72E−01 | 1.98E+00 | 4.48E−06 | 2.69E−05 | 2.05 | 5.00E−04 |
| PANX1 | 0.81 | 4.76E−02 | 1.74E+00 | 3.85E−05 | 2.94E−05 | 2.42 | 5.26E−04 |
| PRRC2A | 0.88 | 5.23E−06 | 7.78E−01 | 7.56E−01 | 2.97E−05 | 0.88 | 5.26E−04 |
| PHF20L1 | 0.91 | 8.52E−03 | 1.73E+00 | 2.15E−04 | 3.33E−05 | 1.52 | 5.75E−04 |
| ACBD6 | 0.93 | 8.77E−04 | 1.72E+00 | 2.18E−03 | 3.60E−05 | 2.16 | 6.05E−04 |
| RBM42 | 1.20 | 2.54E−05 | 1.47E+00 | 1.07E−01 | 3.85E−05 | 1.41 | 6.34E−04 |
| ITSN2 | 1.76 | 6.90E−06 | 6.62E−02 | 8.74E−01 | 4.01E−05 | 1.41 | 6.45E−04 |
| EIF4A2 | −0.85 | 2.00E−05 | −2.05E+00 | 1.61E−01 | 4.20E−05 | −0.97 | 6.59E−04 |
| OPTN | 2.39 | 1.15E−04 | 1.19E+00 | 2.29E−02 | 4.43E−05 | 1.31 | 6.81E−04 |
| RBM17 | 1.01 | 1.47E−05 | 1.50E+00 | 2.76E−01 | 4.63E−05 | 1.02 | 6.96E−04 |
| OST4 | −1.17 | 1.64E−05 | −1.21E+00 | 2.75E−01 | 5.11E−05 | −1.30 | 7.53E−04 |
| NOP9 | 0.55 | 8.29E−02 | 2.08E+00 | 5.06E−05 | 6.03E−05 | 2.00 | 8.53E−04 |
| UBA5 | 2.14 | 2.02E−05 | 4.70E−01 | 2.58E−01 | 5.98E−05 | 1.37 | 8.53E−04 |
| CLPTM1 | 1.25 | 2.83E−04 | 1.24E+00 | 1.31E−02 | 6.33E−05 | 1.80 | 8.77E−04 |
| GPSM2 | 2.50 | 2.30E−04 | 1.40E+00 | 1.75E−02 | 6.72E−05 | 1.37 | 9.13E−04 |
| BLOC1S4 | 1.02 | 5.50E−02 | 1.78E+00 | 8.84E−05 | 7.29E−05 | 2.13 | 9.58E−04 |
| RPL10A | −0.33 | 1.37E−05 | 2.12E−16 | 1.00E+00 | 7.82E−05 | −0.33 | 9.58E−04 |
| WDR70 | 1.95 | 4.88E−05 | 6.94E−01 | 1.11E−01 | 7.34E−05 | 1.79 | 9.58E−04 |
| RPL9 | −0.29 | 1.38E−05 | 2.12E−16 | 1.00E+00 | 7.86E−05 | −0.29 | 9.58E−04 |
| THY1 | 0.93 | 1.43E−05 | 7.57E−01 | 7.48E−01 | 7.77E−05 | 0.95 | 9.58E−04 |
| PGS1 | 1.29 | 3.68E−05 | 9.61E−01 | 1.67E−01 | 7.70E−05 | 1.52 | 9.58E−04 |
| D030056L22RIK | 1.71 | 1.75E−02 | 1.75E+00 | 2.87E−04 | 8.29E−05 | 2.06 | 9.77E−04 |
| CFL1 | 0.38 | 1.45E−05 | 2.00E−01 | 1.00E+00 | 8.25E−05 | 0.38 | 9.77E−04 |
| AMDHD1 | | 1.00E+00 | 3.44E+00 | 8.76E−05 | 8.76E−05 | | 1.01E−03 |
| DUSP2 | 1.36 | 3.83E−03 | 1.57E+00 | 1.56E−03 | 1.02E−04 | 2.46 | 1.17E−03 |
| ZFAND5 | 1.23 | 8.17E−04 | 1.12E+00 | 9.07E−03 | 1.23E−04 | 1.43 | 1.36E−03 |
| POLR2M | 1.08 | 3.84E−05 | 8.14E−01 | 3.03E−01 | 1.23E−04 | 1.26 | 1.36E−03 |
| ZFP78 | −5.85 | 5.20E−05 | −1.19E+00 | 2.07E−01 | 1.26E−04 | −0.24 | 1.37E−03 |
| RPL39 | −0.31 | 2.47E−05 | 2.12E−16 | 1.00E+00 | 1.37E−04 | −0.31 | 1.47E−03 |
| ANKRD49 | 1.15 | 5.68E−04 | −1.02E+00 | 1.85E−02 | 1.64E−04 | −0.40 | 1.73E−03 |
| LRP12 | −2.63 | 3.38E−05 | 1.45E−01 | 7.80E−01 | 1.78E−04 | −0.17 | 1.85E−03 |
| TSEN34 | 0.98 | 1.74E−02 | 1.42E+00 | 6.80E−04 | 1.85E−04 | 2.09 | 1.86E−03 |
| ZBTB38 | 1.35 | 6.47E−03 | 1.38E+00 | 1.74E−03 | 1.82E−04 | 1.90 | 1.86E−03 |
| ATXN2L | 1.05 | 6.53E−05 | 1.10E+00 | 2.81E−01 | 1.93E−04 | 1.10 | 1.90E−03 |
| TPR | 0.93 | 3.75E−05 | 4.02E−01 | 7.25E−01 | 1.92E−04 | 0.95 | 1.90E−03 |
| MGAT2 | 0.97 | 9.70E−03 | 1.50E+00 | 1.28E−03 | 1.97E−04 | 2.07 | 1.91E−03 |
| CD3E | 0.91 | 3.71E−05 | 3.72E−02 | 1.00E+00 | 2.02E−04 | 0.91 | 1.93E−03 |
| MAP3K1 | −1.02 | 1.17E−04 | −1.20E+00 | 1.51E−01 | 2.13E−04 | −1.12 | 2.01E−03 |
| TMEM55B | 1.59 | 6.60E−04 | 9.73E−01 | 2.38E−02 | 2.36E−04 | 1.45 | 2.04E−03 |
| NOMO1 | 1.01 | 1.25E−03 | 1.55E+00 | 1.24E−02 | 2.41E−04 | 1.59 | 2.04E−03 |
| RPS6KC1 | 0.76 | 2.02E−01 | −1.74E+00 | 9.89E−05 | 2.27E−04 | −0.99 | 2.04E−03 |
| FAHD2A | −1.11 | 1.15E−02 | −1.40E+00 | 1.36E−03 | 2.43E−04 | −2.02 | 2.04E−03 |
| SERBP1 | 0.49 | 4.06E−05 | 2.12E−16 | 1.00E+00 | 2.20E−04 | 0.49 | 2.04E−03 |
| CYB5R4 | 1.21 | 4.50E−03 | 1.22E+00 | 3.36E−03 | 2.40E−04 | 1.86 | 2.04E−03 |
| SCAND1 | 0.49 | 1.66E−01 | 1.66E+00 | 1.19E−04 | 2.32E−04 | 1.83 | 2.04E−03 |
| EIF4B | −0.69 | 4.52E−05 | 3.61E−01 | 8.89E−01 | 2.42E−04 | −0.68 | 2.04E−03 |
| EMC7 | 0.51 | 5.74E−03 | 2.99E+00 | 2.65E−03 | 2.41E−04 | 1.09 | 2.04E−03 |
| SIRT1 | −2.52 | 6.79E−05 | 4.37E−01 | 4.03E−01 | 2.52E−04 | −0.18 | 2.10E−03 |
| DUS2 | 1.61 | 3.53E−04 | 8.10E−01 | 5.31E−02 | 2.61E−04 | 1.72 | 2.14E−03 |
| UBE2D3 | 0.57 | 5.48E−05 | 6.10E−01 | 7.65E−01 | 2.80E−04 | 0.57 | 2.23E−03 |
| STYX | 0.82 | 1.81E−02 | 1.52E+00 | 1.01E−03 | 2.76E−04 | 1.42 | 2.23E−03 |
| SDHD | −0.90 | 5.29E−05 | −9.17E−02 | 9.04E−01 | 2.81E−04 | −0.90 | 2.23E−03 |
| YKT6 | 1.05 | 3.87E−04 | −1.05E+00 | 6.13E−02 | 3.20E−04 | 0.48 | 2.51E−03 |
| GM5111 | | 1.00E+00 | 2.97E+00 | 3.54E−04 | 3.54E−04 | | 2.75E−03 |
| EIF3J1 | 1.47 | 1.06E−04 | 5.25E−01 | 3.74E−01 | 3.68E−04 | 1.54 | 2.83E−03 |
| E130309D02RIK | 1.48 | 3.46E−04 | 7.31E−01 | 8.77E−02 | 3.86E−04 | 1.55 | 2.87E−03 |
| EMC10 | 0.53 | 1.17E−01 | 1.61E+00 | 2.65E−04 | 3.79E−04 | 1.76 | 2.87E−03 |
| RPS26 | −0.26 | 7.32E−05 | 2.12E−16 | 1.00E+00 | 3.85E−04 | −0.26 | 2.87E−03 |
| CAMK2D | 0.95 | 1.68E−02 | 1.45E+00 | 1.65E−03 | 4.07E−04 | 1.55 | 2.97E−03 |
| ITGB1 | 0.59 | 1.08E−01 | 1.84E+00 | 3.08E−04 | 4.08E−04 | 1.57 | 2.97E−03 |
| ZFP622 | 1.51 | 1.77E−04 | 5.07E−01 | 2.26E−01 | 4.25E−04 | 1.30 | 3.05E−03 |
| MAPK1 | 0.97 | 3.38E−03 | 1.59E+00 | 8.51E−03 | 4.27E−04 | 1.56 | 3.05E−03 |
| RFNG | 0.47 | 2.04E−01 | 1.60E+00 | 1.94E−04 | 4.31E−04 | 1.94 | 3.05E−03 |
| NPTN | −1.37 | 3.67E−04 | −8.01E−01 | 9.73E−02 | 4.43E−04 | −1.37 | 3.10E−03 |
| RPL7A | −0.28 | 8.68E−05 | 2.12E−16 | 1.00E+00 | 4.52E−04 | −0.28 | 3.13E−03 |
| LYSMD2 | 1.10 | 3.47E−03 | 1.07E+00 | 9.46E−03 | 4.82E−04 | 1.86 | 3.31E−03 |
| VPS37A | 0.90 | 1.41E−02 | 1.43E+00 | 2.70E−03 | 5.47E−04 | 1.58 | 3.67E−03 |
| SLC30A9 | 1.02 | 2.92E−02 | 1.41E+00 | 1.36E−03 | 5.51E−04 | 1.86 | 3.67E−03 |
| RPS21 | −0.35 | 1.06E−04 | 2.12E−16 | 1.00E+00 | 5.44E−04 | −0.35 | 3.67E−03 |
| POLE4 | 0.86 | 8.45E−04 | 1.60E+00 | 5.20E−02 | 5.77E−04 | 1.09 | 3.81E−03 |
| IQGAP1 | 0.95 | 1.40E−04 | −7.52E−01 | 5.66E−01 | 6.03E−04 | 0.93 | 3.95E−03 |
| MTRF1L | 0.61 | 2.51E−01 | 1.69E+00 | 2.40E−04 | 6.09E−04 | 1.55 | 3.95E−03 |
| HDAC9 | −4.55 | 1.23E−04 | 7.41E−02 | 1.00E+00 | 6.31E−04 | −0.26 | 4.05E−03 |
| RPL36A | −0.33 | 1.29E−04 | 2.12E−16 | 1.00E+00 | 6.55E−04 | −0.33 | 4.17E−03 |
| ANKLE2 | 1.22 | 1.88E−03 | 1.07E+00 | 2.56E−02 | 6.61E−04 | 1.49 | 4.17E−03 |
| ABHD12 | 0.16 | 7.30E−01 | 1.66E+00 | 1.42E−04 | 6.79E−04 | 1.86 | 4.24E−03 |
| UBQLN1 | 0.86 | 1.16E−02 | 1.61E+00 | 4.19E−03 | 6.83E−04 | 1.58 | 4.24E−03 |
| SACS | 0.34 | 6.27E−01 | 1.91E+00 | 1.54E−04 | 6.90E−04 | 1.06 | 4.24E−03 |
| YIPF4 | 0.87 | 5.63E−04 | 1.47E+00 | 1.12E−01 | 7.41E−04 | 1.03 | 4.26E−03 |
| ANXA2 | 0.77 | 1.43E−04 | 4.12E−01 | 8.60E−01 | 7.11E−04 | 0.77 | 4.26E−03 |
| A530064D06RIK | 0.43 | 2.35E−02 | 1.47E+00 | 2.25E−03 | 7.22E−04 | 0.38 | 4.26E−03 |
| ARPC1B | 0.43 | 1.44E−04 | 2.12E−16 | 1.00E+00 | 7.27E−04 | 0.43 | 4.26E−03 |
| IFT57 | 0.97 | 2.20E−02 | 1.28E+00 | 2.44E−03 | 7.35E−04 | 1.65 | 4.26E−03 |
| GTF2H5 | 0.88 | 2.01E−04 | −6.71E−01 | 4.17E−01 | 7.17E−04 | 0.75 | 4.26E−03 |
| ATP2B1 | 1.19 | 1.44E−04 | 1.52E−01 | 7.97E−01 | 7.06E−04 | 1.08 | 4.26E−03 |
| HIST2H2AC | 1.63 | 1.81E−02 | 1.78E+00 | 2.94E−03 | 7.33E−04 | 1.24 | 4.26E−03 |
| EEF1A1 | −0.30 | 1.51E−04 | 2.12E−16 | 1.00E+00 | 7.61E−04 | −0.30 | 4.34E−03 |
| DDAH2 | 2.45 | 2.60E−03 | −1.25E+00 | 2.17E−02 | 7.69E−04 | −0.21 | 4.35E−03 |
| AW554918 | 1.86 | 1.63E−04 | −1.42E−01 | 8.09E−01 | 7.94E−04 | 0.26 | 4.37E−03 |
| RAB24 | 0.28 | 5.86E−01 | 1.65E+00 | 1.82E−04 | 7.82E−04 | 1.47 | 4.37E−03 |
| INSL5 | −4.06 | 6.56E−03 | −1.73E+00 | 8.72E−03 | 7.98E−04 | −2.52 | 4.37E−03 |
| THAP11 | 0.64 | 8.01E−02 | 1.47E+00 | 8.15E−04 | 7.97E−04 | 2.06 | 4.37E−03 |
| ASF1A | 0.68 | 5.82E−02 | 1.40E+00 | 1.15E−03 | 8.42E−04 | 1.70 | 4.58E−03 |
| FAM162A | −0.76 | 1.73E−04 | 1.55E−01 | 9.93E−01 | 8.65E−04 | −0.76 | 4.63E−03 |
| WRNIP1 | 1.19 | 3.37E−02 | 1.40E+00 | 1.95E−03 | 8.64E−04 | 1.38 | 4.63E−03 |
| SRM | 0.98 | 2.46E−03 | 1.37E+00 | 2.68E−02 | 8.78E−04 | 1.96 | 4.66E−03 |
| GLIS2 | 4.24 | 9.05E−04 | −1.25E+00 | 8.07E−02 | 8.84E−04 | 0.07 | 4.66E−03 |
| TRIM27 | 1.19 | 7.85E−04 | 9.23E−01 | 9.67E−02 | 8.95E−04 | 1.47 | 4.69E−03 |
| ZDHHC7 | 1.04 | 2.63E−02 | 1.29E+00 | 2.58E−03 | 9.04E−04 | 1.85 | 4.70E−03 |
| CCDC85B | 0.64 | 3.91E−02 | 1.59E+00 | 1.82E−03 | 9.22E−04 | 1.20 | 4.73E−03 |
| KARS | −0.76 | 1.87E−04 | 4.95E−01 | 8.53E−01 | 9.16E−04 | −0.73 | 4.73E−03 |
| ARGLU1 | −0.84 | 1.91E−04 | 3.61E−01 | 9.11E−01 | 9.45E−04 | −0.83 | 4.77E−03 |
| TNP2 | −0.74 | 8.97E−02 | −1.45E+00 | 8.83E−04 | 9.41E−04 | −1.86 | 4.77E−03 |
| CELF1 | 0.94 | 1.99E−04 | 2.75E−01 | 8.30E−01 | 9.64E−04 | 0.94 | 4.80E−03 |
| ALDOA | −0.33 | 1.95E−04 | 2.12E−16 | 1.00E+00 | 9.71E−04 | −0.33 | 4.80E−03 |
| SUMF1 | 0.95 | 1.56E−01 | 1.59E+00 | 5.67E−04 | 9.63E−04 | 1.56 | 4.80E−03 |
| PDCD4 | −0.82 | 4.50E−03 | −2.10E+00 | 1.61E−02 | 9.79E−04 | −1.33 | 4.81E−03 |
| MRPL3 | 0.94 | 9.30E−04 | 1.07E+00 | 9.20E−02 | 1.01E−03 | 1.46 | 4.91E−03 |
| LIMS1 | 0.66 | 3.32E−02 | 1.67E+00 | 2.36E−03 | 1.02E−03 | 1.37 | 4.93E−03 |
| MLF2 | 0.72 | 2.37E−04 | −4.48E−01 | 6.68E−01 | 1.06E−03 | 0.64 | 5.11E−03 |
| RAB33B | 1.30 | 3.19E−04 | −4.65E−01 | 4.41E−01 | 1.14E−03 | 0.94 | 5.46E−03 |
| DBI | 0.76 | 2.41E−04 | 3.96E−01 | 8.92E−01 | 1.17E−03 | 0.76 | 5.55E−03 |
| GFER | 0.78 | 3.95E−03 | −1.76E+00 | 2.32E−02 | 1.19E−03 | 0.07 | 5.63E−03 |
| RAP1A | 0.88 | 2.75E−04 | 7.85E−01 | 7.26E−01 | 1.26E−03 | 0.88 | 5.90E−03 |
| H1F0 | 1.32 | 1.79E−03 | 8.38E−01 | 5.89E−02 | 1.28E−03 | 1.67 | 5.96E−03 |
| XDH | 2.22 | 2.08E−02 | 1.70E+00 | 4.92E−03 | 1.32E−03 | 1.21 | 6.11E−03 |
| APIP | 1.51 | 5.49E−04 | −4.87E−01 | 2.59E−01 | 1.35E−03 | 0.06 | 6.16E−03 |
| ANKRD13A | 0.61 | 1.41E−01 | 1.45E+00 | 8.88E−04 | 1.35E−03 | 1.63 | 6.16E−03 |
| HIST1H3A | −0.73 | 2.46E−01 | 2.23E+00 | 5.88E−04 | 1.39E−03 | 0.78 | 6.28E−03 |
| PAXBP1 | −0.94 | 2.43E−03 | −1.11E+00 | 5.02E−02 | 1.48E−03 | −1.34 | 6.68E−03 |
| 6030458C11RIK | −1.38 | 3.12E−04 | 6.07E−02 | 8.95E−01 | 1.49E−03 | −1.02 | 6.68E−03 |
| FAU | −0.25 | 3.17E−04 | 2.12E−16 | 1.00E+00 | 1.53E−03 | −0.25 | 6.79E−03 |
| ERI1 | 0.58 | 3.08E−02 | 1.76E+00 | 3.98E−03 | 1.54E−03 | 1.12 | 6.79E−03 |
| PRG4 | 1.02 | 4.05E−02 | −1.57E+00 | 3.14E−03 | 1.56E−03 | −0.09 | 6.87E−03 |
| FZR1 | 0.82 | 1.77E−02 | 1.43E+00 | 6.97E−03 | 1.57E−03 | 1.72 | 6.87E−03 |
| CHD7 | 0.91 | 3.47E−04 | −2.77E−01 | 8.59E−01 | 1.64E−03 | 0.88 | 7.10E−03 |
| JUND | 0.73 | 7.14E−04 | 4.85E−01 | 2.45E−01 | 1.66E−03 | 0.61 | 7.15E−03 |
| MIDN | 1.01 | 7.18E−04 | 5.70E−01 | 2.48E−01 | 1.68E−03 | 1.02 | 7.21E−03 |
| EXOSC6 | 0.61 | 2.57E−01 | 1.56E+00 | 7.06E−04 | 1.70E−03 | 1.50 | 7.22E−03 |
| RPS15 | −0.24 | 3.61E−04 | 2.12E−16 | 1.00E+00 | 1.72E−03 | −0.24 | 7.30E−03 |
| OCIAD1 | 0.51 | 3.81E−04 | 6.44E−01 | 7.68E−01 | 1.74E−03 | 0.52 | 7.31E−03 |
| STXBP1 | 0.96 | 1.20E−01 | 1.56E+00 | 1.34E−03 | 1.75E−03 | 1.22 | 7.32E−03 |
| USP39 | 0.88 | 3.80E−03 | 1.44E+00 | 3.80E−02 | 1.76E−03 | 1.27 | 7.32E−03 |
| DAXX | 0.45 | 8.40E−02 | −2.44E+00 | 1.93E−03 | 1.84E−03 | −0.97 | 7.54E−03 |
| RABGAP1 | −1.05 | 1.92E−02 | −1.11E+00 | 7.63E−03 | 1.83E−03 | −1.31 | 7.54E−03 |
| GLB1L | −1.77 | 5.95E−03 | −1.14E+00 | 2.60E−02 | 1.91E−03 | −0.75 | 7.77E−03 |
| HERPUD1 | 1.22 | 4.18E−04 | 1.40E−01 | 7.78E−01 | 1.90E−03 | 1.10 | 7.77E−03 |
| MARC2 | 0.62 | 1.43E−02 | −1.54E+00 | 1.15E−02 | 2.05E−03 | −0.83 | 8.27E−03 |
| RC3H1 | 1.10 | 5.92E−04 | 3.16E−01 | 4.61E−01 | 2.09E−03 | 0.83 | 8.38E−03 |
| DONSON | 1.03 | 4.12E−02 | 1.20E+00 | 4.27E−03 | 2.10E−03 | 1.75 | 8.39E−03 |
| PDGFB | 1.34 | 4.97E−04 | 1.88E−01 | 6.76E−01 | 2.13E−03 | 1.02 | 8.47E−03 |
| RBPJ | −0.66 | 4.55E−04 | 9.10E−02 | 1.00E+00 | 2.14E−03 | −0.66 | 8.47E−03 |
| SPATA5L1 | 0.31 | 6.51E−01 | 1.66E+00 | 5.23E−04 | 2.20E−03 | 1.30 | 8.62E−03 |
| CYP4F17 | −1.50 | 6.37E−02 | −1.98E+00 | 3.02E−03 | 2.21E−03 | −0.41 | 8.62E−03 |
| CHTF18 | 0.66 | 1.16E−01 | 1.66E+00 | 1.86E−03 | 2.29E−03 | 1.32 | 8.86E−03 |
| HIST1H2AC | 4.08 | 7.12E−04 | −6.84E−01 | 4.01E−01 | 2.28E−03 | 0.03 | 8.86E−03 |
| NSUN2 | 0.89 | 1.07E−03 | 8.23E−01 | 2.40E−01 | 2.38E−03 | 1.21 | 9.08E−03 |
| MAPK6 | 0.61 | 4.22E−02 | 2.39E+00 | 4.79E−03 | 2.38E−03 | 1.02 | 9.08E−03 |
| FRMD6 | −1.29 | 5.03E−03 | 9.28E−01 | 4.17E−02 | 2.46E−03 | −0.04 | 9.29E−03 |
| ODC1 | 1.13 | 5.40E−04 | 1.59E−01 | 8.26E−01 | 2.45E−03 | 1.12 | 9.29E−03 |
| CTBP1 | 1.09 | 1.38E−03 | 5.97E−01 | 1.86E−01 | 2.49E−03 | 1.34 | 9.38E−03 |
| PITHD1 | 0.70 | 5.45E−02 | 1.37E+00 | 4.25E−03 | 2.65E−03 | 1.81 | 9.90E−03 |
| SSBP4 | 0.27 | 4.49E−01 | 1.54E+00 | 7.88E−04 | 2.68E−03 | 1.61 | 9.98E−03 |
| RFX3 | 1.77 | 2.63E−03 | −9.27E−01 | 9.65E−02 | 2.72E−03 | −0.02 | 1.00E−02 |
| TRIM24 | 1.30 | 3.48E−03 | 7.69E−01 | 6.99E−02 | 2.71E−03 | 0.80 | 1.00E−02 |
| RPS20 | −0.30 | 5.92E−04 | 2.12E−16 | 1.00E+00 | 2.74E−03 | −0.30 | 1.00E−02 |
| TEC | −1.94 | 6.18E−04 | 1.22E−01 | 8.05E−01 | 2.76E−03 | −0.28 | 1.01E−02 |
| PHLDA3 | 0.96 | 4.66E−02 | 1.24E+00 | 5.55E−03 | 2.95E−03 | 2.09 | 1.06E−02 |
| TRPM3 | −2.12 | 2.73E−03 | 1.35E+00 | 1.03E−01 | 2.96E−03 | −0.01 | 1.06E−02 |
| TRAPPC6B | 1.26 | 7.22E−04 | 2.31E−01 | 6.32E−01 | 2.94E−03 | 1.21 | 1.06E−02 |
| RPL28 | −0.27 | 6.69E−04 | 2.12E−16 | 1.00E+00 | 3.07E−03 | −0.27 | 1.09E−02 |
| 1810026J23RIK | 0.35 | 3.71E−01 | 1.47E+00 | 1.03E−03 | 3.06E−03 | 1.58 | 1.09E−02 |
| GNAI2 | 0.73 | 1.13E−03 | −1.28E+00 | 3.54E−01 | 3.25E−03 | 0.69 | 1.15E−02 |
| MXD4 | 5.26 | 4.14E−03 | 1.78E+00 | 7.29E−02 | 3.28E−03 | 0.74 | 1.15E−02 |
| HPS6 | 1.30 | 1.20E−03 | 4.22E−01 | 3.30E−01 | 3.28E−03 | 1.08 | 1.15E−02 |
| PJA1 | −1.02 | 7.27E−04 | −1.94E−02 | 1.00E+00 | 3.32E−03 | −0.82 | 1.16E−02 |
| TDP2 | 0.52 | 2.81E−01 | 1.41E+00 | 1.39E−03 | 3.37E−03 | 1.84 | 1.17E−02 |
| DGAT2 | 1.60 | 2.17E−01 | −2.78E+00 | 1.79E−03 | 3.55E−03 | −0.35 | 1.22E−02 |
| TMEM102 | −7.18 | 2.07E−03 | 1.64E+00 | 1.92E−01 | 3.71E−03 | −0.01 | 1.27E−02 |
| FBXO2 | 1.52 | 1.66E−02 | 1.20E+00 | 1.99E−02 | 3.78E−03 | 1.45 | 1.29E−02 |
| GM4951 | −2.97 | 3.17E−03 | −8.57E−01 | 1.24E−01 | 3.94E−03 | −0.67 | 1.34E−02 |
| ASPM | 1.19 | 2.17E−03 | 8.42E−01 | 2.00E−01 | 4.01E−03 | 0.52 | 1.36E−02 |
| RPL18 | −0.22 | 9.24E−04 | 2.12E−16 | 1.00E+00 | 4.14E−03 | −0.22 | 1.39E−02 |
| ABHD15 | 4.36 | 3.50E−03 | −1.20E+00 | 1.24E−01 | 4.30E−03 | 0.08 | 1.44E−02 |
| TUSC2 | 0.31 | 5.09E−01 | 1.41E+00 | 1.25E−03 | 4.41E−03 | 1.64 | 1.47E−02 |
| ACTB | −0.36 | 1.00E−03 | 2.12E−16 | 1.00E+00 | 4.46E−03 | −0.36 | 1.48E−02 |
| FBXL22 | −2.73 | 1.06E−03 | 1.69E−01 | 7.93E−01 | 4.54E−03 | −0.31 | 1.50E−02 |
| GTF2A1 | −1.18 | 1.22E−03 | 2.36E−01 | 6.17E−01 | 4.72E−03 | −0.75 | 1.54E−02 |
| CALM1 | 0.45 | 1.28E−03 | −9.16E−01 | 5.59E−01 | 4.71E−03 | 0.43 | 1.54E−02 |
| UPF3A | 0.68 | 1.19E−01 | 1.21E+00 | 4.08E−03 | 4.80E−03 | 1.66 | 1.56E−02 |
| PIK3C2B | −0.56 | 3.39E−01 | −1.58E+00 | 1.82E−03 | 4.91E−03 | −0.62 | 1.59E−02 |
| LRRFIP2 | 0.99 | 1.24E−02 | 8.81E−01 | 3.71E−02 | 5.01E−03 | 1.36 | 1.62E−02 |
| TAF4 | 1.14 | 3.70E−02 | −1.14E+00 | 1.27E−02 | 5.10E−03 | −0.31 | 1.64E−02 |
| TAF2 | −0.20 | 6.11E−01 | 1.36E+00 | 1.35E−03 | 5.15E−03 | 0.66 | 1.65E−02 |
| GGNBP2 | 1.00 | 3.18E−03 | 9.19E−01 | 1.80E−01 | 5.25E−03 | 1.26 | 1.67E−02 |
| GATAD2A | 0.64 | 2.12E−02 | 1.87E+00 | 2.36E−02 | 5.43E−03 | 1.05 | 1.72E−02 |
| GM8096 | | 1.00E+00 | 2.30E+00 | 5.49E−03 | 5.49E−03 | | 1.73E−02 |
| AKR1B3 | 0.57 | 5.83E−03 | 1.51E+00 | 9.45E−02 | 5.52E−03 | 0.82 | 1.74E−02 |
| LTA | −1.13 | 1.61E−03 | −5.70E−01 | 5.32E−01 | 5.69E−03 | −1.17 | 1.78E−02 |
| WDR24 | 0.81 | 2.67E−02 | 1.01E+00 | 2.07E−02 | 5.90E−03 | 1.48 | 1.84E−02 |
| LAPTM4B | 2.03 | 1.40E−03 | −5.19E−02 | 8.89E−01 | 6.02E−03 | 0.52 | 1.87E−02 |
| MARK4 | 1.48 | 2.96E−03 | 5.64E−01 | 2.43E−01 | 6.11E−03 | 0.78 | 1.89E−02 |
| MICU2 | 0.63 | 3.65E−02 | 1.26E+00 | 1.60E−02 | 6.15E−03 | 1.53 | 1.89E−02 |
| RPL12 | −0.25 | 1.43E−03 | 2.12E−16 | 1.00E+00 | 6.18E−03 | −0.25 | 1.89E−02 |
| CCDC12 | 0.81 | 1.98E−03 | 6.55E−01 | 4.49E−01 | 6.28E−03 | 0.93 | 1.92E−02 |
| CCR3 | | 1.00E+00 | 3.05E+00 | 6.51E−03 | 6.51E−03 | | 1.98E−02 |
| UBXN2A | 1.21 | 3.80E−03 | 5.67E−01 | 2.00E−01 | 6.66E−03 | 1.33 | 2.01E−02 |
| CTSF | 4.73 | 6.85E−03 | −1.20E+00 | 1.03E−01 | 6.84E−03 | 0.16 | 2.06E−02 |
| SMARCA5 | 0.53 | 1.50E−01 | 1.36E+00 | 5.66E−03 | 7.71E−03 | 1.36 | 2.31E−02 |
| PLEK | −0.36 | 4.84E−01 | 1.41E+00 | 2.45E−03 | 7.95E−03 | 1.42 | 2.37E−02 |
| GZMB | 1.01 | 1.28E−01 | 1.12E+00 | 6.96E−03 | 8.21E−03 | 2.11 | 2.44E−02 |
| NRM | −1.15 | 4.65E−03 | −5.89E−01 | 2.14E−01 | 8.41E−03 | −1.43 | 2.48E−02 |
| BHLHE40 | −0.73 | 2.03E−03 | 4.47E−01 | 8.55E−01 | 8.42E−03 | −0.73 | 2.48E−02 |
| HIST1H2AH | 2.10 | 3.16E−02 | −1.81E+00 | 2.65E−02 | 8.47E−03 | −0.15 | 2.48E−02 |
| GM5177 | 0.23 | 1.44E−02 | −7.82E−01 | 6.02E−02 | 8.55E−03 | −0.03 | 2.50E−02 |
| PSMD3 | 0.58 | 2.69E−03 | 7.33E−01 | 4.98E−01 | 8.80E−03 | 0.61 | 2.56E−02 |
| ARID3A | 0.75 | 4.66E−02 | 9.95E−01 | 2.02E−02 | 9.30E−03 | 1.01 | 2.69E−02 |
| MRPL11 | 0.55 | 2.12E−02 | −1.14E+00 | 4.45E−02 | 9.32E−03 | −0.04 | 2.69E−02 |
| IL22 | | 1.00E+00 | 2.80E+00 | 9.43E−03 | 9.43E−03 | | 2.71E−02 |
| CANX | −0.44 | 2.27E−03 | 2.12E−16 | 1.00E+00 | 9.46E−03 | −0.44 | 2.71E−02 |
| TMSB10 | 0.35 | 2.34E−03 | 2.12E−16 | 1.00E+00 | 9.76E−03 | 0.35 | 2.77E−02 |
| RPGRIP1 | −0.59 | 8.48E−02 | −1.09E+00 | 1.21E−02 | 9.74E−03 | −1.03 | 2.77E−02 |
| C2CD2L | −1.15 | 8.56E−03 | 6.49E−01 | 1.44E−01 | 1.09E−02 | −0.08 | 3.07E−02 |
| RPS27A | −0.20 | 2.84E−03 | 2.12E−16 | 1.00E+00 | 1.16E−02 | −0.20 | 3.28E−02 |
| RPS10 | −0.21 | 2.91E−03 | 2.12E−16 | 1.00E+00 | 1.19E−02 | −0.21 | 3.32E−02 |
| ZBED6 | 0.67 | 8.45E−02 | −1.05E+00 | 1.53E−02 | 1.19E−02 | −0.28 | 3.32E−02 |
| RPL19 | −0.17 | 2.92E−03 | 2.12E−16 | 1.00E+00 | 1.19E−02 | −0.17 | 3.32E−02 |
| GPR18 | 0.63 | 5.19E−02 | 1.50E+00 | 2.46E−02 | 1.21E−02 | 1.34 | 3.35E−02 |
| GARS | 0.59 | 5.10E−03 | 1.38E+00 | 3.25E−01 | 1.22E−02 | 0.61 | 3.35E−02 |
| MAP2K2 | 0.56 | 1.37E−02 | 2.10E+00 | 9.77E−02 | 1.22E−02 | 0.70 | 3.35E−02 |
| A130010J15RIK | −1.19 | 1.81E−02 | −7.75E−01 | 7.36E−02 | 1.23E−02 | −1.19 | 3.35E−02 |
| RBM3 | −0.36 | 3.02E−03 | 2.12E−16 | 1.00E+00 | 1.23E−02 | −0.36 | 3.35E−02 |
| ISCU | 0.65 | 2.38E−02 | 1.22E+00 | 5.47E−02 | 1.23E−02 | 1.23 | 3.35E−02 |
| ADIPOR1 | 0.78 | 4.40E−03 | 7.02E−01 | 4.16E−01 | 1.25E−02 | 0.90 | 3.37E−02 |
| ITFG2 | 1.22 | 3.84E−03 | 2.67E−01 | 5.33E−01 | 1.26E−02 | 0.50 | 3.40E−02 |
| RPS28 | −0.26 | 3.16E−03 | 2.12E−16 | 1.00E+00 | 1.28E−02 | −0.26 | 3.45E−02 |
| MAX | 0.78 | 3.32E−03 | 1.53E−01 | 8.32E−01 | 1.31E−02 | 0.77 | 3.51E−02 |
| CCDC102A | | 1.00E+00 | −1.97E+00 | 1.35E−02 | 1.35E−02 | | 3.60E−02 |
| RPL23 | −0.18 | 3.47E−03 | 2.12E−16 | 1.00E+00 | 1.40E−02 | −0.18 | 3.71E−02 |
| GBF1 | 0.50 | 1.16E−01 | 1.19E+00 | 1.39E−02 | 1.41E−02 | 1.05 | 3.73E−02 |
| ADAM28 | 0.34 | 7.76E−02 | 9.76E−01 | 2.01E−02 | 1.41E−02 | 0.37 | 3.73E−02 |
| WIPI2 | 0.70 | 2.81E−02 | 1.14E+00 | 6.75E−02 | 1.69E−02 | 1.07 | 4.43E−02 |
| GRINA | 0.62 | 2.20E−02 | −1.30E+00 | 9.03E−02 | 1.73E−02 | −0.24 | 4.54E−02 |
| CDH11 | 0.25 | 8.19E−02 | −1.02E+00 | 2.46E−02 | 1.76E−02 | −0.05 | 4.59E−02 |
| ZFP420 | | 1.00E+00 | 2.22E+00 | 1.77E−02 | 1.77E−02 | | 4.59E−02 |
| SLAIN2 | 1.41 | 8.38E−03 | 4.53E−01 | 2.91E−01 | 1.77E−02 | 0.60 | 4.59E−02 |
| DNA2 | −0.97 | 4.82E−02 | −9.50E−01 | 4.36E−02 | 1.86E−02 | −1.06 | 4.79E−02 |
| TMEM265 | −0.52 | 5.21E−01 | −1.59E+00 | 6.00E−03 | 1.87E−02 | −0.94 | 4.80E−02 |
| APLF | −0.09 | 7.96E−01 | −1.34E+00 | 5.06E−03 | 1.90E−02 | −0.71 | 4.87E−02 |
| DYNC1LI1 | 0.86 | 1.16E−02 | 9.87E−01 | 2.17E−01 | 1.93E−02 | 1.05 | 4.92E−02 |
| SLC7A5 | 0.99 | 9.53E−03 | 4.57E−01 | 3.01E−01 | 2.04E−02 | 0.84 | 5.15E−02 |
| NHLRC2 | 0.81 | 8.24E−02 | 9.32E−01 | 2.91E−02 | 2.04E−02 | 1.28 | 5.15E−02 |
| CBLN3 | 0.59 | 6.31E−03 | −2.45E−01 | 5.69E−01 | 2.04E−02 | 0.21 | 5.15E−02 |
| ZFP109 | 1.91 | 1.09E−01 | 1.49E+00 | 2.24E−02 | 2.05E−02 | 0.36 | 5.15E−02 |
| MRS2 | 0.91 | 1.10E−02 | 5.15E−01 | 2.63E−01 | 2.11E−02 | 1.08 | 5.29E−02 |
| CISD3 | −0.80 | 5.54E−03 | 7.77E−02 | 9.48E−01 | 2.13E−02 | −0.73 | 5.32E−02 |
| HSH2D | −0.63 | 3.75E−01 | −1.47E+00 | 8.74E−03 | 2.17E−02 | −0.53 | 5.40E−02 |
| RPS13 | −0.17 | 5.72E−03 | 2.12E−16 | 1.00E+00 | 2.20E−02 | −0.17 | 5.45E−02 |
| MTX1 | 0.52 | 5.73E−02 | −1.38E+00 | 4.56E−02 | 2.23E−02 | −0.12 | 5.50E−02 |
| BEND4 | 1.02 | 6.41E−03 | 1.86E−01 | 6.93E−01 | 2.25E−02 | 0.87 | 5.54E−02 |
| SLC4A11 | 0.85 | 8.01E−03 | 3.68E−01 | 4.84E−01 | 2.33E−02 | 1.00 | 5.72E−02 |
| NCOA1 | 1.00 | 6.14E−03 | 2.04E−02 | 9.86E−01 | 2.34E−02 | 0.67 | 5.73E−02 |
| HIST2H3C2 | 0.77 | 1.16E−01 | 1.48E+00 | 2.53E−02 | 2.38E−02 | 1.77 | 5.80E−02 |
| MTRF1 | −0.78 | 1.16E−01 | 1.09E+00 | 2.62E−02 | 2.45E−02 | 0.52 | 5.95E−02 |
| CHCHD10 | 0.64 | 8.36E−03 | 7.25E−01 | 5.21E−01 | 2.51E−02 | 0.74 | 6.08E−02 |
| EGFP_IRES_IL23R | 1.59 | 8.29E−02 | 1.35E+00 | 3.70E−02 | 2.52E−02 | 2.43 | 6.09E−02 |
| MMS22L | 0.81 | 4.38E−02 | 8.49E−01 | 7.00E−02 | 2.54E−02 | 1.11 | 6.11E−02 |
| PRDX2 | −0.42 | 6.84E−03 | 2.12E−16 | 1.00E+00 | 2.58E−02 | −0.42 | 6.19E−02 |
| RNR1 | 0.54 | 6.98E−03 | 2.19E−02 | 1.00E+00 | 2.63E−02 | 0.54 | 6.28E−02 |
| RBM43 | −0.48 | 2.17E−01 | −1.01E+00 | 1.73E−02 | 2.75E−02 | −1.28 | 6.54E−02 |
| ZFP944 | −0.98 | 9.89E−03 | 3.41E−01 | 4.68E−01 | 2.76E−02 | −0.46 | 6.54E−02 |
| ZC3H12C | 2.14 | 3.34E−02 | −1.10E+00 | 1.11E−01 | 2.93E−02 | −0.01 | 6.92E−02 |
| PRMT5 | 0.62 | 2.34E−02 | 8.63E−01 | 1.66E−01 | 2.94E−02 | 1.04 | 6.92E−02 |
| DTD1 | 0.81 | 9.24E−03 | −2.67E−01 | 6.19E−01 | 2.98E−02 | 0.25 | 7.01E−02 |
| WDR83 | 0.91 | 1.09E−02 | −3.24E−01 | 4.66E−01 | 3.00E−02 | 0.14 | 7.02E−02 |
| KHNYN | −0.52 | 2.28E−01 | −1.02E+00 | 1.86E−02 | 3.03E−02 | −0.99 | 7.07E−02 |
| NUDCD1 | −1.09 | 8.33E−03 | 4.01E−03 | 9.88E−01 | 3.08E−02 | −0.80 | 7.11E−02 |
| SAA4 | −0.04 | 8.14E−01 | 1.36E+00 | 8.54E−03 | 3.06E−02 | 0.14 | 7.11E−02 |
| PLBD2 | 0.35 | 3.00E−01 | 1.17E+00 | 1.52E−02 | 3.08E−02 | 1.17 | 7.11E−02 |
| GM20939 | −0.80 | 1.48E−02 | −6.14E−01 | 3.32E−01 | 3.20E−02 | −0.92 | 7.36E−02 |
| IFITM2 | −0.66 | 9.52E−03 | 6.88E−01 | 7.07E−01 | 3.23E−02 | −0.62 | 7.40E−02 |
| ACTR2 | −0.41 | 8.79E−03 | 2.12E−16 | 1.00E+00 | 3.23E−02 | −0.41 | 7.40E−02 |
| FURIN | 0.41 | 2.08E−01 | −2.46E+00 | 2.19E−02 | 3.27E−02 | 0.11 | 7.44E−02 |
| SKAP1 | 0.21 | 4.14E−01 | −2.05E+00 | 1.29E−02 | 3.26E−02 | −0.49 | 7.44E−02 |
| SYNGAP1 | 0.81 | 1.56E−01 | −1.00E+00 | 2.86E−02 | 3.33E−02 | −0.16 | 7.51E−02 |
| RBFOX2 | 2.97 | 1.62E−02 | 9.60E−01 | 3.11E−01 | 3.32E−02 | 0.50 | 7.51E−02 |
| PGAM1 | −0.44 | 9.10E−03 | 5.37E−02 | 1.00E+00 | 3.33E−02 | −0.43 | 7.51E−02 |
| GABARAP | −0.49 | 9.14E−03 | 3.72E−02 | 1.00E+00 | 3.35E−02 | −0.49 | 7.51E−02 |
| ATP6V0D2 | −1.33 | 1.28E−02 | 3.65E−01 | 4.44E−01 | 3.37E−02 | −0.33 | 7.55E−02 |
| TRP53I11 | 0.13 | 6.75E−01 | −1.21E+00 | 1.03E−02 | 3.42E−02 | −1.19 | 7.62E−02 |
| ANKRD46 | −0.63 | 2.10E−02 | −7.77E−01 | 2.36E−01 | 3.45E−02 | −0.85 | 7.65E−02 |
| P4HA2 | −0.92 | 2.25E−02 | −5.29E−01 | 2.17E−01 | 3.45E−02 | −1.24 | 7.65E−02 |
| ARL6IP5 | −0.45 | 6.55E−02 | −2.03E+00 | 6.86E−02 | 3.50E−02 | −0.75 | 7.72E−02 |
| GM10416 | 1.46 | 3.85E−02 | −9.71E−01 | 1.21E−01 | 3.54E−02 | −0.08 | 7.79E−02 |
| TCTEX1D1 | −2.53 | 1.35E−02 | 6.12E−01 | 4.52E−01 | 3.56E−02 | −0.05 | 7.82E−02 |
| PGP | 0.73 | 1.66E−02 | 5.52E−01 | 3.39E−01 | 3.60E−02 | 0.98 | 7.87E−02 |
| ATP5G2 | −0.29 | 1.03E−02 | 2.12E−16 | 1.00E+00 | 3.71E−02 | −0.29 | 8.10E−02 |
| GYPC | 0.89 | 1.40E−02 | −3.57E−01 | 4.66E−01 | 3.73E−02 | 0.48 | 8.11E−02 |
| TAGAP | 0.93 | 7.93E−02 | −8.82E−01 | 6.16E−02 | 3.74E−02 | −0.27 | 8.11E−02 |
| SLC17A9 | −0.11 | 7.46E−01 | −1.54E+00 | 1.12E−02 | 3.80E−02 | −0.78 | 8.22E−02 |
| COX15 | 0.22 | 6.24E−01 | −1.11E+00 | 1.23E−02 | 3.86E−02 | −0.37 | 8.33E−02 |
| LGALS4 | −1.06 | 1.33E−02 | 2.38E−01 | 5.47E−01 | 3.89E−02 | −0.16 | 8.34E−02 |
| PABPC4 | 0.71 | 4.66E−02 | −8.71E−01 | 1.11E−01 | 3.89E−02 | −0.05 | 8.34E−02 |
| CDCA7 | −0.11 | 7.64E−01 | −1.49E+00 | 1.16E−02 | 3.96E−02 | −1.01 | 8.46E−02 |
| CTSD | 0.57 | 1.89E−02 | 1.40E+00 | 3.33E−01 | 3.99E−02 | 0.62 | 8.49E−02 |
| NCAPH2 | −0.24 | 2.77E−01 | −2.29E+00 | 2.19E−02 | 4.01E−02 | −0.54 | 8.51E−02 |
| ARPC3 | 0.54 | 1.28E−02 | 8.12E−01 | 6.35E−01 | 4.02E−02 | −0.51 | 8.51E−02 |
| STAU1 | 0.65 | 2.56E−02 | 6.46E−01 | 2.32E−01 | 4.06E−02 | 0.90 | 8.57E−02 |
| RINL | 0.74 | 3.24E−02 | −6.96E−01 | 1.79E−01 | 4.11E−02 | −0.05 | 8.64E−02 |
| CCDC184 | 0.48 | 5.50E−01 | −1.22E+00 | 1.41E−02 | 4.12E−02 | −0.54 | 8.65E−02 |
| AGPAT5 | 0.42 | 1.68E−01 | 1.24E+00 | 3.64E−02 | 4.34E−02 | 0.45 | 9.07E−02 |
| PAIP1 | −0.51 | 7.01E−02 | −9.91E−01 | 8.39E−02 | 4.35E−02 | −0.80 | 9.08E−02 |
| AKNA | 0.69 | 1.39E−02 | 1.83E−01 | 7.35E−01 | 4.58E−02 | 0.70 | 9.53E−02 |
| MDFIC | 0.79 | 1.33E−02 | 2.70E−02 | 9.58E−01 | 4.66E−02 | 0.65 | 9.65E−02 |
| ZBTB7A | 0.74 | 1.71E−02 | −4.46E−01 | 5.11E−01 | 4.69E−02 | 0.62 | 9.70E−02 |
| KLF6 | 0.69 | 3.43E−02 | −8.59E−01 | 2.08E−01 | 4.83E−02 | 0.41 | 9.95E−02 |
| TRAF3IP3 | 0.44 | 1.57E−01 | 1.26E+00 | 4.43E−02 | 4.86E−02 | 1.23 | 9.98E−02 |
| PDE4B | 0.86 | 4.37E−02 | 7.72E−01 | 1.60E−01 | 4.87E−02 | 1.17 | 9.98E−02 |

TABLE 1D
Th17 Ctrl GFP+ vs GFP−

| Gene | coefC | pvalC | coefD | pvalD | pvalH | logFC | FDR |
|---|---|---|---|---|---|---|---|
| IL23R | 1.41 | 3.62E−02 | 4.02E+00 | 3.92E−22 | 5.36E−22 | 4.29 | 7.04E−18 |
| OST4 | 1.71 | 3.76E−16 | 9.93E−01 | 6.10E−01 | 3.42E−15 | 1.74 | 2.25E−11 |
| EGFP_IRES_IL23R | 3.10 | 1.03E−02 | 3.77E+00 | 7.06E−14 | 2.50E−14 | 2.90 | 1.10E−10 |
| LARS2 | −0.93 | 5.68E−15 | 1.18E−16 | 1.00E+00 | 5.65E−14 | −0.93 | 1.86E−10 |
| SDHD | 1.48 | 1.43E−13 | 1.20E+00 | 5.22E−01 | 1.10E−12 | 1.49 | 2.90E−09 |
| CALM2 | 1.06 | 1.55E−13 | 1.18E−16 | 1.00E+00 | 1.46E−12 | 1.06 | 3.20E−09 |
| IZUMO1R | −1.61 | 5.82E−07 | −2.57E+00 | 4.20E−07 | 1.05E−11 | −3.67 | 1.96E−08 |
| IL12RB2 | 1.84 | 1.27E−08 | 2.35E+00 | 4.54E−05 | 2.29E−11 | 3.02 | 3.77E−08 |
| RORA | 1.18 | 2.84E−03 | 2.25E+00 | 2.86E−09 | 2.54E−10 | 2.30 | 3.71E−07 |
| NPTN | 1.80 | 2.52E−09 | 1.33E+00 | 3.87E−03 | 2.98E−10 | 2.18 | 3.92E−07 |
| PRRC2A | −1.13 | 1.27E−10 | −1.80E+00 | 1.55E−01 | 3.80E−10 | −1.16 | 4.54E−07 |
| LSM4 | −1.25 | 1.58E−10 | 1.08E+00 | 2.78E−01 | 7.21E−10 | −1.10 | 7.90E−07 |
| CCDC71L | 0.85 | 3.78E−02 | 2.34E+00 | 9.40E−10 | 8.55E−10 | 2.12 | 8.65E−07 |
| CASP6 | 1.43 | 5.71E−10 | 1.20E+00 | 1.16E−01 | 1.32E−09 | 1.71 | 1.24E−06 |
| RNH1 | 0.94 | 1.93E−10 | 1.18E−16 | 1.00E+00 | 1.57E−09 | 0.94 | 1.38E−06 |
| COX17 | 0.93 | 2.98E−10 | 5.81E−01 | 8.63E−01 | 2.37E−09 | 0.93 | 1.95E−06 |
| OSTF1 | 1.45 | 1.04E−08 | 1.87E+00 | 2.32E−02 | 5.82E−09 | 1.86 | 4.50E−06 |
| EMB | 1.22 | 1.31E−09 | 1.08E+00 | 4.87E−01 | 8.01E−09 | 1.24 | 5.85E−06 |
| SMOX | 1.66 | 2.99E−08 | 1.61E+00 | 1.60E−02 | 1.17E−08 | 2.34 | 8.13E−06 |
| NCS1 | 1.49 | 1.97E−09 | −3.45E−01 | 5.45E−01 | 1.26E−08 | 1.32 | 8.32E−06 |
| TRIP10 | 1.31 | 4.41E−06 | 1.55E+00 | 1.11E−04 | 1.51E−08 | 2.13 | 9.45E−06 |
| RAD50 | −1.26 | 6.40E−09 | −6.14E−01 | 2.40E−01 | 2.40E−08 | −1.36 | 1.44E−05 |
| TIAM1 | −1.42 | 2.41E−04 | −1.66E+00 | 4.28E−06 | 3.04E−08 | −1.68 | 1.74E−05 |
| TIMP1 | 2.32 | 3.00E−03 | 1.83E+00 | 9.54E−07 | 7.44E−08 | 3.87 | 4.08E−05 |
| HIVEP3 | −1.51 | 1.94E−07 | −8.81E−01 | 1.77E−02 | 7.87E−08 | −1.62 | 4.14E−05 |
| S1PR1 | 1.25 | 1.96E−05 | 1.58E+00 | 1.56E−04 | 8.66E−08 | 2.33 | 4.38E−05 |
| ARGLU1 | 1.09 | 1.53E−08 | 1.08E−01 | 1.00E+00 | 1.12E−07 | 1.08 | 5.46E−05 |
| BHLHE40 | 1.43 | 2.00E−07 | 1.35E+00 | 6.42E−02 | 2.43E−07 | 1.77 | 1.14E−04 |
| PDGFA | 1.46 | 4.10E−02 | 2.29E+00 | 3.65E−07 | 2.99E−07 | 2.01 | 1.31E−04 |
| AHCYL2 | −1.13 | 9.11E−05 | −1.73E+00 | 1.21E−04 | 2.92E−07 | 2.05 | 1.31E−04 |
| IL4 | −1.42 | 1.60E−02 | −1.90E+00 | 9.98E−07 | 3.50E−07 | −2.52 | 1.48E−04 |
| SBNO2 | 1.28 | 1.22E−07 | 1.70E+00 | 2.00E−01 | 3.68E−07 | 1.34 | 1.51E−04 |
| NOL7 | −1.13 | 4.33E−06 | −1.81E+00 | 5.20E−03 | 5.25E−07 | −1.69 | 2.09E−04 |
| H2-Q10 | 1.84 | 2.31E−05 | 1.13E+00 | 1.40E−03 | 7.84E−07 | 2.61 | 2.79E−04 |
| FLOT1 | 1.14 | 3.82E−05 | 2.08E+00 | 8.11E−04 | 7.62E−07 | 1.92 | 2.79E−04 |
| LGALS1 | 0.85 | 1.12E−07 | 1.18E−16 | 1.00E+00 | 7.71E−07 | 0.85 | 2.79E−04 |
| TSPO | 0.82 | 1.44E−07 | 1.07E+00 | 4.86E−01 | 7.71E−07 | 0.86 | 2.79E−04 |
| JUNB | 0.93 | 1.46E−07 | 1.18E−16 | 1.00E+00 | 9.95E−07 | 0.93 | 3.42E−04 |
| TMEM258 | 0.70 | 1.49E−07 | 1.18E−16 | 1.00E+00 | 1.01E−06 | 0.70 | 3.42E−04 |
| ACTB | 0.53 | 1.66E−07 | 1.18E−16 | 1.00E+00 | 1.13E−06 | 0.53 | 3.70E−04 |
| IL17F | 7.41 | 1.04E−04 | 1.75E+00 | 4.86E−04 | 1.22E−06 | 2.68 | 3.92E−04 |
| NPC2 | 0.82 | 2.04E−07 | 1.18E−16 | 1.00E+00 | 1.38E−06 | 0.82 | 4.31E−04 |
| UBASH3A | −0.85 | 6.02E−03 | −1.59E+00 | 1.32E−05 | 1.73E−06 | −2.33 | 4.81E−04 |
| PKIB | 1.34 | 8.54E−02 | 2.19E+00 | 1.18E−06 | 1.70E−06 | 1.20 | 4.81E−04 |
| STAT5A | 1.11 | 2.72E−07 | 8.05E−01 | 7.53E−01 | 1.73E−06 | 1.12 | 4.81E−04 |
| RPS2 | −0.78 | 2.68E−07 | 1.18E−16 | 1.00E+00 | 1.79E−06 | −0.78 | 4.81E−04 |
| GNB5 | 0.55 | 6.89E−02 | 1.88E+00 | 1.43E−06 | 1.72E−06 | 2.48 | 4.81E−04 |
| TPT1 | −0.39 | 2.69E−07 | 1.18E−16 | 1.00E+00 | 1.79E−06 | −0.39 | 4.81E−04 |
| RNF187 | −1.40 | 1.45E−06 | −1.00E+00 | 6.32E−02 | 1.62E−06 | −1.82 | 4.81E−04 |
| INPP5B | 1.24 | 5.23E−07 | 7.84E−01 | 2.75E−01 | 1.88E−06 | 1.43 | 4.95E−04 |
| NMT1 | 1.15 | 3.77E−06 | 1.85E+00 | 2.76E−02 | 2.02E−06 | 1.32 | 5.20E−04 |
| ARHGDIA | 0.47 | 3.30E−07 | 1.18E−16 | 1.00E+00 | 2.19E−06 | 0.47 | 5.47E−04 |
| LTB | −1.61 | 1.35E−06 | −7.33E−01 | 1.01E−01 | 2.21E−06 | −1.95 | 5.47E−04 |
| TREML2 | −1.13 | 3.47E−05 | −1.11E+00 | 3.46E−03 | 2.64E−06 | −1.84 | 6.44E−04 |
| PABPC1 | −0.55 | 4.21E−07 | 1.18E−16 | 1.00E+00 | 2.77E−06 | −0.55 | 6.62E−04 |
| SH2D3C | −1.34 | 7.23E−05 | −1.13E+00 | 1.74E−03 | 2.83E−06 | −1.82 | 6.64E−04 |
| MT2 | 1.69 | 6.27E−06 | 8.36E−01 | 2.80E−02 | 3.32E−06 | 2.37 | 7.67E−04 |
| NDUFA1 | 0.75 | 9.18E−07 | 1.36E+00 | 3.20E−01 | 3.58E−06 | 0.80 | 7.85E−04 |
| TMEM160 | −1.20 | 1.04E−05 | −9.29E−01 | 1.75E−02 | 3.56E−06 | −1.79 | 7.85E−04 |
| RNF10 | −1.21 | 9.36E−06 | −1.25E+00 | 1.93E−02 | 3.52E−06 | −1.56 | 7.85E−04 |
| MYCN | −1.36 | 2.00E−01 | 2.53E+00 | 1.36E−06 | 3.77E−06 | 1.41 | 8.00E−04 |
| SMIM3 | 0.56 | 1.37E−01 | 1.76E+00 | 1.81E−06 | 3.73E−06 | 2.64 | 8.00E−04 |
| ILTIFB | | 1.00E+00 | 3.62E+00 | 4.09E−06 | 4.09E−06 | | 8.16E−04 |
| MTMR7 | 0.88 | 8.76E−03 | 1.60E+00 | 2.28E−05 | 4.09E−06 | 2.09 | 8.16E−04 |
| H2-T23 | 0.68 | 6.16E−07 | 1.18E−16 | 1.00E+00 | 3.99E−06 | 0.68 | 8.16E−04 |
| SUN2 | −1.20 | 3.48E−06 | −7.46E−01 | 7.00E−02 | 4.08E−06 | −1.50 | 8.16E−04 |
| PTPN14 | 1.30 | 1.81E−02 | 2.10E+00 | 1.36E−05 | 4.78E−06 | 0.75 | 9.39E−04 |
| SOCS3 | 1.22 | 2.93E−04 | 1.76E+00 | 7.74E−04 | 4.99E−06 | 2.20 | 9.66E−04 |
| IL22 | 5.60 | 6.60E−03 | 2.47E+00 | 3.89E−05 | 5.27E−06 | 1.93 | 1.01E−03 |
| ITSN2 | −1.03 | 3.75E−04 | −1.43E+00 | 7.03E−04 | 5.75E−06 | −1.74 | 1.05E−03 |
| D17H6S53E | 1.09 | 1.12E−05 | 9.75E−01 | 2.79E−02 | 5.77E−06 | 1.30 | 1.05E−03 |
| ANXA2 | 1.18 | 8.81E−07 | 1.18E−16 | 1.00E+00 | 5.64E−06 | 1.18 | 1.05E−03 |
| TUBA1A | 0.97 | 2.13E−06 | 1.65E+00 | 2.19E−01 | 6.19E−06 | 1.09 | 1.12E−03 |
| RPS28 | 0.38 | 1.02E−06 | 1.18E−16 | 1.00E+00 | 6.46E−06 | 0.38 | 1.15E−03 |
| ERMN | 1.44 | 2.99E−04 | 1.35E+00 | 1.09E−03 | 7.00E−06 | 2.52 | 1.23E−03 |
| SPHK1 | −0.96 | 3.38E−01 | 2.32E+00 | 1.87E−06 | 7.34E−06 | 1.08 | 1.27E−03 |
| SRRM2 | −0.76 | 1.22E−06 | 1.18E−16 | 1.00E+00 | 7.70E−06 | −0.76 | 1.32E−03 |
| TRIM27 | −1.39 | 4.25E−06 | −6.99E−01 | 1.34E−01 | 8.34E−06 | −1.52 | 1.39E−03 |
| ACSL4 | 0.98 | 1.85E−06 | −1.16E+00 | 4.23E−01 | 8.37E−06 | 0.94 | 1.39E−03 |
| CRISPLD2 | 1.07 | 2.52E−01 | 2.14E+00 | 2.80E−06 | 8.87E−06 | 1.47 | 1.46E−03 |
| MS4A6D | 2.75 | 1.76E−06 | 2.58E−01 | 5.34E−01 | 9.03E−06 | 0.71 | 1.47E−03 |
| ADAM8 | 1.71 | 1.20E−03 | 1.35E+00 | 3.63E−04 | 9.16E−06 | 1.66 | 1.47E−03 |
| PRDX2 | 0.58 | 1.54E−06 | 1.18E−16 | 1.00E+00 | 9.64E−06 | 0.58 | 1.53E−03 |
| SNAPC1 | 0.74 | 1.50E−03 | 3.11E+00 | 3.31E−04 | 1.03E−05 | 1.42 | 1.61E−03 |
| HSD17B10 | 0.82 | 1.67E−06 | 3.40E−01 | 1.00E+00 | 1.06E−05 | 0.86 | 1.62E−03 |
| SEPT2 | 0.90 | 2.16E−06 | 1.08E+00 | 4.87E−01 | 1.05E−05 | 0.92 | 1.62E−03 |
| CKS2 | 0.78 | 4.87E−06 | 1.82E+00 | 1.59E−01 | 1.08E−05 | 0.90 | 1.63E−03 |
| CLK4 | −0.40 | 1.53E−01 | 2.26E+00 | 5.11E−06 | 1.10E−05 | 1.15 | 1.64E−03 |
| ATXN1 | −1.11 | 1.82E−06 | 4.97E−02 | 1.00E+00 | 1.14E−05 | −0.98 | 1.69E−03 |
| CBX3 | 0.78 | 1.89E−06 | 1.18E−16 | 1.00E+00 | 1.17E−05 | 0.78 | 1.72E−03 |
| UNC50 | 1.07 | 4.64E−06 | 1.02E+00 | 1.92E−01 | 1.19E−05 | 1.32 | 1.72E−03 |
| RASGRP1 | −0.75 | 1.73E−02 | −1.59E+00 | 4.30E−05 | 1.37E−05 | −1.63 | 1.94E−03 |
| TOMM7 | 0.56 | 2.22E−06 | 1.18E−16 | 1.00E+00 | 1.37E−05 | 0.56 | 1.94E−03 |
| PRPF8 | −0.72 | 2.68E−06 | −1.73E−01 | 8.53E−01 | 1.61E−05 | −0.72 | 2.26E−03 |
| IL16 | −0.11 | 7.04E−01 | −1.83E+00 | 2.95E−06 | 1.68E−05 | −1.87 | 2.32E−03 |
| GPR34 | | 1.00E+00 | −3.67E+00 | 1.85E−05 | 1.85E−05 | | 2.53E−03 |
| S100A10 | 0.87 | 3.33E−06 | 1.18E−16 | 1.00E+00 | 2.02E−05 | 0.87 | 2.69E−03 |
| FCF1 | 0.74 | 1.77E−05 | 2.10E+00 | 7.21E−02 | 1.98E−05 | 0.96 | 2.69E−03 |
| WNK1 | −0.76 | 6.78E−06 | −1.55E+00 | 2.41E−01 | 2.01E−05 | −0.79 | 2.69E−03 |
| SERPINE1 | 0.62 | 3.12E−01 | 1.87E+00 | 6.49E−06 | 2.30E−05 | 1.15 | 3.02E−03 |
| GIMAP4 | −0.78 | 5.99E−04 | −2.61E+00 | 2.15E−03 | 2.50E−05 | −1.32 | 3.25E−03 |
| D630039A03RIK | −1.44 | 1.69E−02 | −1.73E+00 | 9.27E−05 | 2.77E−05 | −1.50 | 3.57E−03 |
| RAB37 | 0.48 | 6.02E−01 | −2.32E+00 | 5.98E−06 | 3.09E−05 | −1.04 | 3.95E−03 |
| GPBP1L1 | −1.16 | 2.08E−05 | 7.34E−01 | 1.20E−01 | 3.49E−05 | −0.67 | 4.42E−03 |
| ZBTB7C | −0.78 | 5.08E−01 | 2.83E+00 | 8.21E−06 | 3.85E−05 | 0.61 | 4.75E−03 |
| IL1R1 | 0.76 | 6.58E−01 | 2.78E+00 | 7.17E−06 | 3.83E−05 | 0.82 | 4.75E−03 |
| NOTCH1 | −0.86 | 8.77E−06 | −5.79E−01 | 4.55E−01 | 3.87E−05 | −0.89 | 4.75E−03 |
| LUM | 2.31 | 1.70E−03 | 1.27E+00 | 1.23E−03 | 3.93E−05 | 2.08 | 4.79E−03 |
| NANOG | 0.86 | 1.05E−05 | 3.80E−01 | 3.71E−01 | 4.07E−05 | 0.82 | 4.91E−03 |
| LY6A | 0.86 | 7.69E−06 | 1.18E−16 | 1.00E+00 | 4.51E−05 | 0.85 | 5.40E−03 |
| IL17A | 3.32 | 3.20E−01 | 2.57E+00 | 1.31E−05 | 4.60E−05 | 2.42 | 5.42E−03 |
| RAD23A | 0.93 | 2.81E−05 | 1.91E+00 | 1.19E−01 | 4.62E−05 | 1.06 | 5.42E−03 |
| GSK3B | 0.87 | 1.38E−05 | 1.46E+00 | 3.15E−01 | 4.74E−05 | 0.92 | 5.52E−03 |
| REST | −1.16 | 2.55E−05 | −5.71E−01 | 1.44E−01 | 4.87E−05 | −1.21 | 5.62E−03 |
| WTAP | 0.75 | 7.17E−04 | 2.79E+00 | 3.85E−03 | 5.02E−05 | 1.10 | 5.74E−03 |
| CYP11A1 | −1.32 | 3.22E−03 | −1.16E+00 | 8.76E−04 | 5.14E−05 | −2.26 | 5.75E−03 |
| RPSA | −0.37 | 8.85E−06 | 1.18E−16 | 1.00E+00 | 5.16E−05 | −0.37 | 5.75E−03 |
| CYSTM1 | 0.95 | 6.59E−05 | 1.16E+00 | 5.00E−02 | 5.10E−05 | 1.46 | 5.75E−03 |
| TNFSF9 | 1.64 | 1.28E−03 | 1.11E+00 | 2.26E−03 | 5.29E−05 | 1.75 | 5.84E−03 |
| TNFAIP3 | 1.07 | 6.62E−04 | 1.44E+00 | 4.54E−03 | 5.41E−05 | 1.80 | 5.93E−03 |
| CD3E | −0.86 | 9.85E−06 | 1.18E−16 | 1.00E+00 | 5.71E−05 | −0.86 | 6.19E−03 |
| SDF2 | 0.81 | 4.79E−04 | 2.03E+00 | 6.77E−03 | 5.74E−05 | 1.40 | 6.19E−03 |
| SDC4 | 0.14 | 7.01E−01 | 1.66E+00 | 1.08E−05 | 5.81E−05 | 1.64 | 6.21E−03 |
| CRIP1 | 0.77 | 2.08E−04 | 2.53E+00 | 1.76E−02 | 6.17E−05 | 1.06 | 6.49E−03 |
| MYLK | 1.04 | 5.23E−01 | 2.95E+00 | 1.32E−05 | 6.15E−05 | 0.31 | 6.49E−03 |
| GATM | 1.41 | 1.91E−04 | 1.05E+00 | 2.04E−02 | 6.45E−05 | 2.05 | 6.73E−03 |
| ACTR2 | 0.50 | 1.13E−05 | 1.18E−16 | 1.00E+00 | 6.52E−05 | 0.50 | 6.75E−03 |
| RPGRIP1 | 0.56 | 2.42E−02 | 1.40E+00 | 1.67E−04 | 6.60E−05 | 1.24 | 6.78E−03 |
| REPS1 | −0.86 | 1.72E−02 | −1.31E+00 | 2.49E−04 | 7.13E−05 | −1.40 | 7.27E−03 |
| TNFRSF18 | −0.86 | 1.25E−05 | 1.18E−16 | 1.00E+00 | 7.18E−05 | −0.86 | 7.27E−03 |
| PLAGL2 | −0.86 | 1.47E−05 | 7.98E−01 | 6.56E−01 | 7.56E−05 | −0.84 | 7.59E−03 |
| KHSRP | −0.96 | 1.61E−05 | −2.22E−01 | 5.67E−01 | 7.74E−05 | −0.76 | 7.71E−03 |
| GATA3 | −1.00 | 7.13E−04 | −2.28E+00 | 6.31E−03 | 7.82E−05 | −1.45 | 7.73E−03 |
| TRAT1 | −0.68 | 5.61E−02 | −1.42E+00 | 9.49E−05 | 7.93E−05 | −2.63 | 7.78E−03 |
| APOA1BP | 0.83 | 1.24E−04 | 1.27E+00 | 4.22E−02 | 8.05E−05 | 1.34 | 7.79E−03 |
| ATXN2L | −1.01 | 1.41E−05 | −1.13E−02 | 9.57E−01 | 8.05E−05 | −1.00 | 7.79E−03 |
| POU2AF1 | −0.39 | 6.49E−01 | −2.32E+00 | 1.64E−05 | 8.38E−05 | −0.59 | 8.04E−03 |
| CCR8 | −1.04 | 5.80E−03 | −1.52E+00 | 8.50E−04 | 8.52E−05 | −2.45 | 8.12E−03 |
| SRRM1 | −0.65 | 1.58E−05 | 1.18E−16 | 1.00E+00 | 8.99E−05 | −0.64 | 8.51E−03 |
| GNGT2 | 0.89 | 1.03E−04 | 9.36E−01 | 6.18E−02 | 9.32E−05 | 1.58 | 8.76E−03 |
| RERE | −0.45 | 5.12E−02 | −1.76E+00 | 1.26E−04 | 9.63E−05 | −1.18 | 8.98E−03 |
| ZFP467 | 0.60 | 2.73E−01 | −1.77E+00 | 3.25E−05 | 9.75E−05 | −0.89 | 9.03E−03 |
| LMNA | 0.88 | 2.02E−02 | 1.28E+00 | 3.19E−04 | 1.03E−04 | 2.10 | 9.52E−03 |
| AMICA1 | −1.37 | 1.44E−01 | −2.19E+00 | 5.69E−05 | 1.04E−04 | −1.17 | 9.52E−03 |
| KLF6 | 0.68 | 4.05E−03 | 1.70E+00 | 1.52E−03 | 1.06E−04 | 1.71 | 9.57E−03 |
| ASB2 | −1.17 | 3.00E−02 | −1.29E+00 | 2.45E−04 | 1.14E−04 | −1.78 | 1.03E−02 |
| UBR4 | −0.82 | 3.59E−05 | 1.45E+00 | 3.06E−01 | 1.16E−04 | −0.78 | 1.04E−02 |
| RPS27 | 0.35 | 2.09E−05 | 1.18E−16 | 1.00E+00 | 1.17E−04 | 0.35 | 1.04E−02 |
| ABCB9 | −0.82 | 1.64E−02 | −1.23E+00 | 4.46E−04 | 1.18E−04 | −1.71 | 1.04E−02 |
| RORC | 0.75 | 5.29E−02 | 1.34E+00 | 1.57E−04 | 1.22E−04 | 1.88 | 1.07E−02 |
| CCDC88C | −0.86 | 5.25E−05 | 5.04E−01 | 2.18E−01 | 1.31E−04 | −0.43 | 1.14E−02 |
| TRIM56 | −0.26 | 2.35E−01 | −1.50E+00 | 5.28E−05 | 1.39E−04 | −0.63 | 1.20E−02 |
| LAMA5 | −0.90 | 3.98E−04 | −1.16E+00 | 2.23E−02 | 1.39E−04 | −1.22 | 1.20E−02 |
| EIF3F | −0.83 | 2.56E−05 | 1.18E−16 | 1.00E+00 | 1.42E−04 | −0.83 | 1.21E−02 |
| STOM | 0.04 | 9.29E−01 | 1.56E+00 | 2.69E−05 | 1.48E−04 | 2.01 | 1.25E−02 |
| BEND4 | 0.27 | 4.95E−01 | 1.50E+00 | 3.42E−05 | 1.48E−04 | 1.17 | 1.25E−02 |
| ITPRIPL1 | 0.83 | 2.84E−05 | 8.11E−01 | 7.68E−01 | 1.50E−04 | 0.84 | 1.25E−02 |
| CDKN2D | 0.74 | 5.62E−05 | 9.31E−01 | 2.38E−01 | 1.49E−04 | 1.00 | 1.25E−02 |
| CCR4 | −0.95 | 1.33E−04 | −9.50E−01 | 8.34E−02 | 1.51E−04 | −1.32 | 1.25E−02 |
| KMT2A | −0.77 | 8.23E−05 | −7.95E−01 | 1.57E−01 | 1.58E−04 | −0.87 | 1.30E−02 |
| MT1 | 0.93 | 2.33E−03 | 1.66E+00 | 4.34E−03 | 1.66E−04 | 1.64 | 1.35E−02 |
| NUBP1 | −0.89 | 3.11E−05 | −8.06E−02 | 8.41E−01 | 1.68E−04 | −0.80 | 1.35E−02 |
| MAP3K1 | 0.80 | 2.08E−04 | 1.12E+00 | 5.68E−02 | 1.68E−04 | 0.98 | 1.35E−02 |
| RPS12 | −0.41 | 3.11E−05 | 1.18E−16 | 1.00E+00 | 1.71E−04 | −0.41 | 1.37E−02 |
| DTWD1 | 1.39 | 3.72E−05 | 2.58E−01 | 5.97E−01 | 1.76E−04 | 1.23 | 1.40E−02 |
| GTF2IRD1 | −0.14 | 7.41E−01 | −1.56E+00 | 3.48E−05 | 1.80E−04 | −0.68 | 1.43E−02 |
| USMG5 | 0.49 | 3.33E−05 | 1.18E−16 | 1.00E+00 | 1.82E−04 | 0.49 | 1.44E−02 |
| DNMT3A | −0.90 | 1.41E−04 | −8.99E−01 | 1.02E−01 | 1.87E−04 | −1.05 | 1.47E−02 |
| PERP | 0.68 | 7.71E−02 | 1.47E+00 | 1.86E−04 | 1.94E−04 | 2.44 | 1.51E−02 |
| EGFR | −0.45 | 3.58E−01 | 1.47E+00 | 5.65E−05 | 1.97E−04 | 1.39 | 1.53E−02 |
| ASAH2 | | 1.00E+00 | 3.37E+00 | 2.00E−04 | 2.00E−04 | | 1.53E−02 |
| TNIP2 | 0.97 | 3.43E−04 | 1.28E+00 | 4.15E−02 | 2.06E−04 | 1.42 | 1.57E−02 |
| RND1 | 0.83 | 3.29E−02 | 1.27E+00 | 4.41E−04 | 2.14E−04 | 1.25 | 1.62E−02 |
| SYTL1 | −0.15 | 6.14E−01 | −1.50E+00 | 4.64E−05 | 2.20E−04 | −1.96 | 1.66E−02 |
| TCEB1 | 0.63 | 4.33E−05 | 1.18E−16 | 1.00E+00 | 2.34E−04 | 0.63 | 1.76E−02 |
| FERMT3 | 0.66 | 4.42E−05 | 1.18E−16 | 1.00E+00 | 2.39E−04 | 0.66 | 1.78E−02 |
| BYSL | 0.71 | 4.90E−03 | 2.16E+00 | 3.08E−03 | 2.39E−04 | 1.33 | 1.78E−02 |
| TWF1 | 0.85 | 1.64E−04 | 1.16E+00 | 1.16E−01 | 2.41E−04 | 1.13 | 1.78E−02 |
| EIF4EBP2 | −0.90 | 5.11E−03 | −1.11E+00 | 3.03E−03 | 2.45E−04 | −1.71 | 1.80E−02 |
| DIAPH1 | −0.71 | 5.96E−03 | −1.30E+00 | 2.62E−03 | 2.46E−04 | −1.31 | 1.80E−02 |
| RPS20 | −0.37 | 4.64E−05 | 1.18E−16 | 1.00E+00 | 2.50E−04 | −0.37 | 1.81E−02 |
| FYB | −0.74 | 1.07E−04 | −1.21E+00 | 2.11E−01 | 2.53E−04 | −0.85 | 1.83E−02 |
| EP300 | −0.62 | 5.27E−03 | −1.58E+00 | 3.17E−03 | 2.62E−04 | −1.06 | 1.88E−02 |
| CHD4 | −0.71 | 6.06E−05 | −6.09E−01 | 5.41E−01 | 2.67E−04 | −0.72 | 1.89E−02 |
| SETD5 | −0.90 | 1.53E−04 | −1.01E+00 | 1.44E−01 | 2.66E−04 | −1.01 | 1.89E−02 |
| CAPN3 | −3.22 | 4.49E−02 | −2.50E+00 | 4.20E−04 | 2.66E−04 | −0.62 | 1.89E−02 |
| FAM60A | 1.06 | 6.50E−05 | 8.04E−01 | 4.82E−01 | 2.68E−04 | 1.15 | 1.89E−02 |
| TRBC2 | −0.94 | 6.01E−05 | −6.29E−01 | 5.67E−01 | 2.71E−04 | −1.03 | 1.89E−02 |
| FAM102A | −0.20 | 5.55E−01 | −1.44E+00 | 6.60E−05 | 2.93E−04 | −1.44 | 2.04E−02 |
| KPNA2 | 0.72 | 1.33E−04 | 1.25E+00 | 1.97E−01 | 2.95E−04 | 0.89 | 2.04E−02 |
| HN1 | 0.57 | 5.69E−05 | 1.18E−16 | 1.00E+00 | 3.03E−04 | 0.57 | 2.06E−02 |
| CHURC1 | 0.74 | 5.63E−05 | 1.18E−16 | 1.00E+00 | 3.00E−04 | 0.74 | 2.06E−02 |
| GPR83 | −1.30 | 1.01E−02 | −1.10E+00 | 1.94E−03 | 3.02E−04 | −1.51 | 2.06E−02 |
| BCL2A1B | −1.05 | 2.94E−04 | −6.65E−01 | 8.05E−02 | 3.10E−04 | −1.67 | 2.08E−02 |
| GSG1L | | 1.00E+00 | −3.15E+00 | 3.11E−04 | 3.11E−04 | | 2.08E−02 |
| SMG7 | −0.76 | 1.80E−03 | −1.42E+00 | 1.14E−02 | 3.12E−04 | −1.11 | 2.08E−02 |
| ATRNL1 | 2.19 | 1.61E−03 | 1.24E+00 | 1.28E−02 | 3.11E−04 | 0.48 | 2.08E−02 |
| MYL12A | 0.47 | 5.92E−05 | 1.18E−16 | 1.00E+00 | 3.15E−04 | 0.47 | 2.09E−02 |
| GGNBP2 | −0.85 | 2.13E−04 | −9.13E−01 | 1.22E−01 | 3.19E−04 | −1.05 | 2.11E−02 |
| VEZT | −0.87 | 1.85E−04 | 5.14E−01 | 1.49E−01 | 3.26E−04 | −0.12 | 2.14E−02 |
| D930015E06RIK | −0.81 | 7.05E−03 | −1.10E+00 | 3.04E−03 | 3.29E−04 | −1.30 | 2.15E−02 |
| LRRC2 | 0.25 | 2.79E−02 | 1.24E+00 | 8.23E−04 | 3.32E−04 | 0.39 | 2.16E−02 |
| PNO1 | 0.23 | 3.17E−01 | 3.35E+00 | 1.07E−04 | 3.35E−04 | 0.92 | 2.17E−02 |
| HSF2 | 0.78 | 5.33E−03 | 1.68E+00 | 4.15E−03 | 3.39E−04 | 1.29 | 2.18E−02 |
| SCOC | 0.87 | 3.39E−04 | 6.47E−01 | 7.71E−02 | 3.41E−04 | 1.27 | 2.19E−02 |
| MED12 | −0.83 | 6.61E−05 | −5.86E−02 | 8.75E−01 | 3.45E−04 | −0.79 | 2.20E−02 |
| COTL1 | −0.80 | 7.53E−05 | 8.72E−01 | 6.20E−01 | 3.49E−04 | −0.78 | 2.22E−02 |
| ATP6V0E | 0.56 | 6.84E−05 | 5.76E−01 | 8.26E−01 | 3.52E−04 | 0.57 | 2.23E−02 |
| ZHX1 | −0.21 | 4.28E−01 | 2.10E+00 | 9.70E−05 | 3.67E−04 | 0.78 | 2.30E−02 |
| PRPF19 | −0.77 | 1.69E−04 | 6.25E−01 | 1.95E−01 | 3.67E−04 | −0.53 | 2.30E−02 |
| EEF1D | 0.47 | 7.05E−05 | 1.18E−16 | 1.00E+00 | 3.71E−04 | 0.47 | 2.30E−02 |
| NUP210 | −0.73 | 3.74E−04 | −2.13E+00 | 7.63E−02 | 3.71E−04 | −0.81 | 2.30E−02 |
| CHMP5 | 0.62 | 1.20E−04 | 1.49E+00 | 3.24E−01 | 3.77E−04 | 0.70 | 2.31E−02 |
| SUPT4A | 0.60 | 2.76E−04 | −1.76E+00 | 1.11E−01 | 3.77E−04 | 0.52 | 2.31E−02 |
| NEDD8 | 0.54 | 7.51E−05 | 7.81E−01 | 7.54E−01 | 3.75E−04 | 0.56 | 2.31E−02 |
| EZH2 | −1.08 | 2.16E−04 | −7.60E−01 | 1.53E−01 | 3.84E−04 | −1.27 | 2.34E−02 |
| TESC | −0.36 | 2.94E−01 | −1.37E+00 | 1.41E−04 | 4.10E−04 | −1.94 | 2.49E−02 |
| DYNLT1C | 1.08 | 8.26E−05 | 2.83E−01 | 8.06E−01 | 4.18E−04 | 1.11 | 2.52E−02 |
| BRMS1 | 0.81 | 1.82E−04 | 6.85E−01 | 2.19E−01 | 4.27E−04 | 1.14 | 2.55E−02 |
| RAB24 | −1.12 | 1.67E−04 | −3.93E−01 | 2.46E−01 | 4.27E−04 | −0.82 | 2.55E−02 |
| KDELR2 | 0.77 | 8.27E−05 | 1.18E−16 | 1.00E+00 | 4.32E−04 | 0.77 | 2.57E−02 |
| CDC37 | 0.35 | 8.35E−05 | 1.18E−16 | 1.00E+00 | 4.35E−04 | 0.35 | 2.58E−02 |
| AKNA | −0.59 | 1.64E−02 | −2.16E+00 | 1.85E−03 | 4.40E−04 | −1.09 | 2.60E−02 |
| TUBA1C | 0.52 | 1.23E−02 | 1.97E+00 | 2.50E−03 | 4.51E−04 | 1.05 | 2.65E−02 |
| AHCY | −0.73 | 1.60E−04 | −1.46E+00 | 2.97E−01 | 4.67E−04 | −0.79 | 2.73E−02 |
| MCTP2 | −0.14 | 7.16E−01 | 1.49E+00 | 9.93E−05 | 4.80E−04 | 1.06 | 2.79E−02 |
| NDUFB8 | −0.60 | 1.27E−04 | 1.15E+00 | 4.46E−01 | 4.84E−04 | −0.55 | 2.80E−02 |
| SHTN1 | −1.08 | 5.43E−02 | −1.57E+00 | 7.17E−04 | 5.13E−04 | −0.56 | 2.96E−02 |
| DPYSL2 | 0.78 | 1.59E−04 | −1.32E+00 | 3.48E−01 | 5.16E−04 | 0.75 | 2.96E−02 |
| PLEKHJ1 | 0.51 | 1.74E−03 | 2.47E+00 | 2.11E−02 | 5.20E−04 | 0.77 | 2.97E−02 |
| MED14 | −0.22 | 5.51E−01 | −1.36E+00 | 1.25E−04 | 5.34E−04 | −1.00 | 3.04E−02 |
| SNHG11 | 0.47 | 6.03E−04 | 7.75E−01 | 6.99E−02 | 5.39E−04 | 0.48 | 3.05E−02 |
| H1F0 | −1.14 | 3.26E−04 | −5.40E−01 | 1.47E−01 | 5.49E−04 | −1.24 | 3.10E−02 |
| AAK1 | −0.80 | 1.08E−04 | −1.06E−02 | 9.64E−01 | 5.56E−04 | −0.63 | 3.11E−02 |
| ATP1A1 | −0.87 | 1.32E−03 | −8.58E−01 | 3.04E−02 | 5.54E−04 | −1.23 | 3.11E−02 |
| STXBP3 | 0.84 | 5.13E−04 | 6.72E−01 | 8.82E−02 | 5.60E−04 | 1.30 | 3.12E−02 |
| SKIV2L | −0.60 | 2.38E−03 | 1.21E+00 | 1.66E−02 | 5.62E−04 | 0.15 | 3.12E−02 |
| TMEM8B | −0.91 | 2.72E−01 | −2.01E+00 | 2.15E−04 | 5.80E−04 | −0.19 | 3.18E−02 |
| H2-Q4 | −1.06 | 1.17E−04 | 2.15E−01 | 7.99E−01 | 5.80E−04 | −1.02 | 3.18E−02 |
| HCN3 | −0.44 | 7.55E−01 | 2.71E+00 | 1.19E−04 | 5.80E−04 | 0.53 | 3.18E−02 |
| GPR18 | 0.80 | 9.91E−03 | 1.14E+00 | 4.10E−03 | 5.84E−04 | 2.01 | 3.18E−02 |
| NAA15 | −0.81 | 9.38E−04 | −1.14E+00 | 4.75E−02 | 5.89E−04 | −1.10 | 3.20E−02 |
| GALE | 0.70 | 9.38E−04 | 1.11E+00 | 4.77E−02 | 5.92E−04 | 1.34 | 3.20E−02 |
| SEC61G | 0.53 | 1.16E−04 | 1.18E−16 | 1.00E+00 | 5.94E−04 | 0.53 | 3.20E−02 |
| MLLT3 | −1.01 | 1.64E−04 | −3.61E−01 | 4.23E−01 | 5.97E−04 | −0.98 | 3.20E−02 |
| TRIM28 | −0.68 | 1.40E−03 | −1.32E+00 | 3.17E−02 | 6.04E−04 | −1.06 | 3.23E−02 |
| CHP1 | 0.72 | 1.21E−04 | 1.95E−01 | 1.00E+00 | 6.23E−04 | 0.73 | 3.27E−02 |
| ECHDC1 | 0.82 | 6.67E−03 | 1.11E+00 | 6.45E−03 | 6.18E−04 | 1.67 | 3.27E−02 |
| SLC39A8 | 0.52 | 2.11E−01 | 1.28E+00 | 2.79E−04 | 6.20E−04 | 1.50 | 3.27E−02 |
| ZFAND5 | −0.68 | 4.48E−02 | −1.17E+00 | 1.05E−03 | 6.23E−04 | −1.09 | 3.27E−02 |
| ANP32B | −0.93 | 4.10E−04 | −1.18E+00 | 1.30E−01 | 6.19E−04 | −1.07 | 3.27E−02 |
| RAP1A | −0.66 | 1.28E−04 | −1.72E−01 | 8.12E−01 | 6.33E−04 | −0.66 | 3.29E−02 |
| ZMIZ1 | −0.84 | 1.40E−04 | −2.37E−01 | 6.31E−01 | 6.33E−04 | −0.82 | 3.29E−02 |
| PLSCR1 | 0.96 | 2.18E−04 | 6.32E−01 | 3.04E−01 | 6.36E−04 | 1.14 | 3.29E−02 |
| YTHDF3 | −1.16 | 1.38E−04 | 1.10E−01 | 7.99E−01 | 6.76E−04 | −0.78 | 3.48E−02 |
| RAB11FIP4 | 1.05 | 9.02E−02 | −1.48E+00 | 6.34E−04 | 6.94E−04 | −0.42 | 3.57E−02 |
| D830030K20RIK | | 1.00E+00 | −3.19E+00 | 7.00E−04 | 7.00E−04 | | 3.58E−02 |
| BC031181 | 0.68 | 3.93E−04 | 1.78E+00 | 1.63E−01 | 7.07E−04 | 0.79 | 3.61E−02 |
| GNPDA2 | 1.13 | 2.65E−04 | 4.75E−01 | 2.81E−01 | 7.21E−04 | 1.36 | 3.66E−02 |
| SLC12A7 | 0.54 | 8.12E−02 | 1.18E+00 | 7.33E−04 | 7.30E−04 | 1.26 | 3.68E−02 |
| MAGED1 | 1.04 | 3.85E−02 | −1.66E+00 | 1.44E−03 | 7.30E−04 | −0.54 | 3.68E−02 |
| ALKBH5 | 0.77 | 4.01E−04 | 1.74E+00 | 1.72E−01 | 7.49E−04 | 0.81 | 3.76E−02 |
| CYSLTR2 | 0.83 | 1.11E−02 | 1.26E+00 | 4.91E−03 | 7.59E−04 | 1.78 | 3.78E−02 |
| ZIC5 | | 1.00E+00 | 3.09E+00 | 7.59E−04 | 7.59E−04 | | 3.78E−02 |
| CD209C | −0.29 | 2.68E−01 | 1.29E+00 | 2.92E−04 | 7.65E−04 | 0.54 | 3.78E−02 |
| TNFRSF4 | −0.73 | 5.37E−04 | −1.91E+00 | 1.23E−01 | 7.62E−04 | −0.88 | 3.78E−02 |
| SLC4A11 | 0.34 | 2.54E−01 | 1.47E+00 | 3.05E−04 | 7.70E−04 | 1.85 | 3.79E−02 |
| KLHL6 | 0.94 | 3.90E−04 | 9.03E−01 | 1.87E−01 | 7.75E−04 | 1.25 | 3.81E−02 |
| BCKDHA | 0.65 | 5.10E−03 | 1.09E+00 | 1.14E−02 | 8.03E−04 | 1.54 | 3.90E−02 |
| TANGO2 | 0.92 | 1.96E−02 | 1.07E+00 | 3.00E−03 | 8.04E−04 | 1.81 | 3.90E−02 |
| TREX1 | 0.55 | 2.54E−02 | 1.90E+00 | 2.33E−03 | 8.01E−04 | 1.63 | 3.90E−02 |
| CTXN1 | 0.40 | 7.00E−01 | 2.16E+00 | 1.75E−04 | 8.14E−04 | 0.74 | 3.94E−02 |
| ERCC4 | 0.95 | 1.85E−03 | −8.03E−01 | 3.37E−02 | 8.23E−04 | 0.28 | 3.96E−02 |
| LFNG | −0.37 | 6.72E−01 | −1.91E+00 | 1.83E−04 | 8.36E−04 | −0.52 | 4.01E−02 |
| UCK2 | −0.87 | 1.45E−03 | −1.19E+00 | 4.55E−02 | 8.48E−04 | −1.31 | 4.05E−02 |
| MRGPRA6 | −0.36 | 1.70E−04 | 1.18E−16 | 1.00E+00 | 8.52E−04 | −0.36 | 4.06E−02 |
| SLAIN1 | −1.15 | 7.18E−03 | −9.75E−01 | 8.68E−03 | 8.61E−04 | −0.96 | 4.09E−02 |
| EML3 | −0.75 | 6.28E−03 | 1.15E+00 | 1.01E−02 | 8.73E−04 | 0.11 | 4.13E−02 |
| ACOT8 | 0.42 | 1.14E−01 | 1.37E+00 | 6.76E−04 | 8.90E−04 | 1.70 | 4.16E−02 |
| LAMP1 | −0.82 | 4.20E−03 | −1.04E+00 | 1.57E−02 | 8.98E−04 | −1.61 | 4.16E−02 |
| BRCA2 | −0.86 | 9.09E−03 | −1.08E+00 | 7.19E−03 | 8.98E−04 | −0.76 | 4.16E−02 |
| RPS6KA4 | −0.01 | 9.74E−01 | 1.52E+00 | 1.79E−04 | 8.93E−04 | 1.38 | 4.16E−02 |
| HIST1H1E | −0.71 | 7.99E−03 | −9.86E−01 | 8.16E−03 | 8.97E−04 | −1.58 | 4.16E−02 |
| PAPSS2 | 2.10 | 2.19E−04 | −2.67E−01 | 5.30E−01 | 8.88E−04 | 0.23 | 4.16E−02 |
| EIF5B | −0.54 | 1.81E−04 | 4.15E−01 | 1.00E+00 | 9.04E−04 | −0.54 | 4.17E−02 |
| SMC4 | −0.86 | 1.83E−04 | 1.26E−01 | 9.93E−01 | 9.12E−04 | −0.83 | 4.19E−02 |
| TLR7 | | 1.00E+00 | −3.06E+00 | 9.19E−04 | 9.19E−04 | | 4.21E−02 |
| MTA1 | −0.79 | 6.77E−04 | −6.03E−01 | 1.19E−01 | 9.23E−04 | −0.92 | 4.21E−02 |
| SEC23B | 0.81 | 1.93E−04 | −8.51E−02 | 8.05E−01 | 9.32E−04 | 0.77 | 4.23E−02 |
| RN7S2 | −0.79 | 2.03E−04 | −1.49E−01 | 7.01E−01 | 9.35E−04 | −0.71 | 4.23E−02 |
| RN7S1 | −0.79 | 2.03E−04 | −1.49E−01 | 7.01E−01 | 9.35E−04 | −0.71 | 4.23E−02 |
| CHRNA1 | 0.45 | 4.87E−04 | 5.16E−01 | 1.82E−01 | 9.39E−04 | 0.45 | 4.23E−02 |
| ANKRD17 | −0.79 | 2.20E−04 | −3.57E−01 | 6.03E−01 | 9.47E−04 | −0.80 | 4.25E−02 |
| CTS8-PS | | 1.00E+00 | −3.09E+00 | 9.50E−04 | 9.50E−04 | | 4.25E−02 |
| HUWE1 | −0.64 | 2.71E−04 | −1.16E+00 | 4.23E−01 | 9.58E−04 | −0.65 | 4.26E−02 |
| SLC43A1 | −3.09 | 1.49E−01 | −2.43E+00 | 5.84E−04 | 9.55E−04 | −0.50 | 4.26E−02 |
| SLC43A2 | −0.04 | 9.01E−01 | −1.36E+00 | 1.97E−04 | 9.71E−04 | −0.71 | 4.30E−02 |
| PRR12 | −0.34 | 2.00E−01 | −1.28E+00 | 4.72E−04 | 9.77E−04 | −0.58 | 4.31E−02 |
| 1110037F02RIK | −0.98 | 2.90E−04 | −3.06E−01 | 4.04E−01 | 9.94E−04 | −0.86 | 4.37E−02 |
| ATP10D | 0.12 | 7.14E−01 | 1.32E+00 | 2.19E−04 | 1.01E−03 | 0.83 | 4.43E−02 |
| HPCAL4 | | 1.00E+00 | 3.01E+00 | 1.03E−03 | 1.03E−03 | | 4.51E−02 |
| CPSF4 | −0.98 | 2.33E−04 | −1.80E−01 | 6.61E−01 | 1.04E−03 | −0.89 | 4.53E−02 |
| FGF2 | −0.70 | 7.29E−01 | −2.62E+00 | 2.26E−04 | 1.05E−03 | −0.51 | 4.54E−02 |
| HNRNPAB | −0.84 | 6.61E−04 | −1.02E+00 | 1.46E−01 | 1.05E−03 | −1.05 | 4.55E−02 |
| SOX5 | −0.23 | 4.26E−01 | 1.35E+00 | 3.02E−04 | 1.06E−03 | 0.62 | 4.59E−02 |
| ENSA | 0.63 | 2.62E−02 | 1.48E+00 | 3.15E−03 | 1.08E−03 | 1.25 | 4.64E−02 |
| DOK2 | −0.70 | 1.51E−02 | −1.69E+00 | 5.36E−03 | 1.08E−03 | −1.45 | 4.64E−02 |
| ZFP622 | −0.97 | 2.42E−03 | −7.35E−01 | 3.58E−02 | 1.11E−03 | −1.02 | 4.72E−02 |
| TUBB4B | 0.48 | 2.84E−03 | 2.37E+00 | 3.01E−02 | 1.11E−03 | 0.74 | 4.72E−02 |
| PRKCB | −0.24 | 4.15E−01 | −1.33E+00 | 3.24E−04 | 1.12E−03 | −1.06 | 4.74E−02 |
| NUDT3 | 0.51 | 2.26E−02 | 2.78E+00 | 3.77E−03 | 1.12E−03 | 0.80 | 4.74E−02 |
| FABP5 | 0.77 | 4.00E−04 | −8.10E−01 | 3.08E−01 | 1.13E−03 | 0.62 | 4.76E−02 |
| SLC15A3 | 1.09 | 1.42E−03 | 9.35E−01 | 6.62E−02 | 1.14E−03 | 1.70 | 4.78E−02 |
| CCNT1 | −0.83 | 7.75E−04 | 7.48E−01 | 1.34E−01 | 1.14E−03 | −0.16 | 4.78E−02 |
| SYMPK | −0.68 | 5.07E−03 | −1.21E+00 | 1.70E−02 | 1.14E−03 | −1.02 | 4.78E−02 |
| STK11 | −0.97 | 5.46E−04 | 4.53E−01 | 2.11E−01 | 1.16E−03 | −0.15 | 4.83E−02 |
| PON3 | 2.13 | 8.53E−04 | 8.38E−01 | 1.26E−01 | 1.19E−03 | 0.35 | 4.95E−02 |
| PGLYRP2 | −1.12 | 3.78E−02 | −1.23E+00 | 2.50E−03 | 1.20E−03 | −1.15 | 4.95E−02 |
| VEZF1 | −0.84 | 3.05E−03 | −8.57E−01 | 3.14E−02 | 1.23E−03 | −1.17 | 5.05E−02 |
| KMO | | 1.00E+00 | −3.13E+00 | 1.24E−03 | 1.24E−03 | | 5.08E−02 |
| PAIP1 | 0.68 | 1.90E−03 | 1.14E+00 | 5.34E−02 | 1.25E−03 | 0.96 | 5.09E−02 |
| LRRN4CL | −0.28 | 5.48E−02 | −1.08E+00 | 1.86E−03 | 1.25E−03 | −0.42 | 5.09E−02 |
| CYTIP | 0.55 | 2.56E−04 | 1.18E−16 | 1.00E+00 | 1.25E−03 | 0.55 | 5.09E−02 |
| TPST1 | −0.71 | 3.56E−01 | −1.63E+00 | 4.11E−04 | 1.27E−03 | −0.76 | 5.15E−02 |
| HNRNPU | −0.76 | 4.24E−04 | −9.77E−01 | 3.40E−01 | 1.27E−03 | −0.80 | 5.15E−02 |
| CXCR2 | 0.64 | 2.68E−04 | 2.88E−01 | 8.27E−01 | 1.28E−03 | 0.65 | 5.15E−02 |
| PHGDH | −0.70 | 2.90E−04 | 7.78E−01 | 6.87E−01 | 1.30E−03 | −0.68 | 5.18E−02 |
| SLAMF6 | −0.06 | 9.38E−01 | −1.37E+00 | 2.67E−04 | 1.30E−03 | −1.39 | 5.18E−02 |
| DNTTIP1 | −1.28 | 7.47E−04 | −5.04E−01 | 1.65E−01 | 1.29E−03 | −0.71 | 5.18E−02 |
| GZMB | 0.44 | 5.20E−01 | −1.59E+00 | 3.33E−04 | 1.30E−03 | −0.98 | 5.19E−02 |
| MRPL27 | 0.75 | 2.37E−03 | 1.44E+00 | 4.44E−02 | 1.31E−03 | 1.10 | 5.19E−02 |
| FBXO46 | −0.90 | 1.84E−03 | 7.61E−01 | 5.92E−02 | 1.32E−03 | −0.15 | 5.22E−02 |
| CCDC63 | −0.46 | 2.96E−01 | 1.33E+00 | 4.91E−04 | 1.33E−03 | 0.85 | 5.26E−02 |
| CELF1 | −0.75 | 3.95E−04 | −8.32E−01 | 4.12E−01 | 1.34E−03 | −0.77 | 5.28E−02 |
| TCF20 | −0.74 | 8.17E−04 | −1.41E+00 | 1.59E−01 | 1.37E−03 | −0.79 | 5.37E−02 |
| TRIM17 | | 1.00E+00 | −3.03E+00 | 1.38E−03 | 1.38E−03 | | 5.40E−02 |
| IL10RA | −0.19 | 6.76E−01 | −1.25E+00 | 3.13E−04 | 1.38E−03 | −1.39 | 5.40E−02 |
| TPD52 | 0.28 | 1.86E−01 | 3.03E+00 | 7.30E−04 | 1.39E−03 | 0.90 | 5.41E−02 |
| PTPN1 | 0.65 | 5.36E−04 | 1.50E+00 | 2.87E−01 | 1.42E−03 | 0.71 | 5.49E−02 |
| CREBBP | −0.82 | 5.38E−04 | −4.53E−01 | 2.89E−01 | 1.43E−03 | −0.87 | 5.53E−02 |
| ZFP704 | −0.17 | 8.59E−01 | −2.49E+00 | 3.01E−04 | 1.43E−03 | −0.05 | 5.53E−02 |
| MCUR1 | −1.13 | 3.33E−04 | −1.34E−01 | 6.80E−01 | 1.47E−03 | −0.52 | 5.63E−02 |
| TUBB6 | 0.76 | 1.88E−02 | 1.17E+00 | 6.06E−03 | 1.46E−03 | 1.92 | 5.63E−02 |
| TAF2 | −0.66 | 1.42E−02 | −9.90E−01 | 8.02E−03 | 1.47E−03 | −1.09 | 5.64E−02 |
| NT5C | 0.55 | 4.46E−04 | 1.21E+00 | 4.06E−01 | 1.49E−03 | 0.59 | 5.64E−02 |
| S100A13 | 0.51 | 1.38E−02 | 2.62E+00 | 8.34E−03 | 1.48E−03 | 0.98 | 5.64E−02 |
| KMT2C | −0.74 | 5.12E−04 | −4.10E−01 | 3.30E−01 | 1.49E−03 | −0.72 | 5.64E−02 |
| PPP6C | 0.63 | 1.71E−03 | 1.55E+00 | 7.58E−02 | 1.51E−03 | 0.86 | 5.69E−02 |
| SRGN | 0.53 | 3.13E−04 | 1.18E−16 | 1.00E+00 | 1.51E−03 | 0.53 | 5.69E−02 |
| AHDC1 | −0.02 | 9.47E−01 | −1.29E+00 | 3.13E−04 | 1.51E−03 | −0.48 | 5.69E−02 |
| GDPD2 | | 1.00E+00 | 3.28E+00 | 1.52E−03 | 1.52E−03 | | 5.71E−02 |
| ZFP65 | −0.51 | 3.22E−04 | 1.18E−16 | 1.00E+00 | 1.55E−03 | −0.51 | 5.74E−02 |
| FBXO22 | 1.18 | 4.01E−04 | 3.00E−01 | 5.23E−01 | 1.55E−03 | 1.17 | 5.74E−02 |
| TRAPPC2L | 0.67 | 5.55E−03 | 1.10E+00 | 2.22E−02 | 1.57E−03 | 1.60 | 5.74E−02 |
| EPAS1 | −1.29 | 1.26E−02 | −9.26E−01 | 9.68E−03 | 1.57E−03 | −1.01 | 5.74E−02 |
| RELT | 0.03 | 9.46E−01 | −1.31E+00 | 3.23E−04 | 1.55E−03 | −1.21 | 5.74E−02 |
| RXRA | −0.32 | 5.82E−01 | −1.32E+00 | 3.81E−04 | 1.56E−03 | −0.98 | 5.74E−02 |
| ZFP41 | 0.33 | 4.82E−01 | −1.55E+00 | 4.16E−04 | 1.54E−03 | −0.84 | 5.74E−02 |
| MYL6 | 0.35 | 3.25E−04 | 1.18E−16 | 1.00E+00 | 1.56E−03 | 0.34 | 5.74E−02 |
| RPL5 | −0.22 | 3.27E−04 | 1.18E−16 | 1.00E+00 | 1.57E−03 | −0.22 | 5.74E−02 |
| SNX3 | −0.39 | 3.30E−04 | 1.18E−16 | 1.00E+00 | 1.59E−03 | −0.39 | 5.79E−02 |
| CCDC66 | −1.26 | 3.42E−04 | 8.65E−02 | 8.23E−01 | 1.60E−03 | −0.70 | 5.82E−02 |
| DCLRE1C | 0.78 | 5.07E−04 | 1.21E+00 | 3.86E−01 | 1.63E−03 | 0.80 | 5.90E−02 |
| BAG6 | −0.69 | 4.49E−04 | −3.49E−01 | 4.72E−01 | 1.63E−03 | −0.75 | 5.91E−02 |
| NSMCE4A | 0.07 | 8.71E−01 | −1.32E+00 | 3.56E−04 | 1.68E−03 | −1.22 | 5.94E−02 |
| GOLT1B | 0.77 | 1.12E−03 | 1.86E+00 | 1.43E−01 | 1.69E−03 | 0.88 | 5.94E−02 |
| ACSL3 | −1.01 | 4.95E−03 | −7.79E−01 | 2.73E−02 | 1.69E−03 | −1.15 | 5.94E−02 |
| CLIC5 | 1.70 | 2.11E−01 | 2.27E+00 | 7.99E−04 | 1.65E−03 | 0.25 | 5.94E−02 |
| UBE3A | −0.68 | 2.13E−02 | −1.18E+00 | 6.15E−03 | 1.65E−03 | −1.24 | 5.94E−02 |

Table 2A-2G. Tables related to the 10×, single-cell sequencing on T cells derived from the in-vivo mouse experiments. Table 2A-2F—results of Tissue vs. Tissue differential expression comparisons (both between-tissue comparisons and within-tissue, genotype comparisons) Table 2G—Tissue combined.

Table Description and Headings:

Spleen Ctrl vs KG: Genotype comparison between cells extracted from the Spleen. Positive log FC associated with higher expression in Ctrl cells (compared with KG Cells).

Colon LPL Ctrl vs KO: Genotype comparison between cells extracted from the Colon Lamina Propria. Positive log FC associated with higher expression in Ctrl cells (compared with KO Cells).

Small Intestine LPL Ctrl vs KO: Genotype comparison between cells extracted from the Small Intestine Lamina Propria. Positive log FC associated with higher expression in Ctrl cells (compared with KO Cells).

Colon IEL Ctrl vs KO: Genotype comparison between cells extracted from the Colon Epithelium. Positive log FC associated with higher expression in Ctrl cells (compared with KO Cells).

LPL vs Spleen: Comparison between cells taken from the Lamina Propria (Colon and Small Intestine) vs. Spleen. Positive log FC associated with higher expression in Lamina Propria.

IEL vs Spleen: Comparison between cells taken from the Colon Epithelium vs. Spleen. Positive log FC associated with higher expression in Colon Epithelium.

Tissue Combined: ANOVA-like test for tissue-specific expression. Tests the inclusion of all tissue coefficients simultaneously. Reference level—Spleen.

GeneID: Unique Ensembl identifier for the gene being tested

GeneSymbol: Common symbol used for this gene

log FC: log 2 coefficient of the linear model

log CPM: log 2 counts-per-million average expression

LR: likelihood ratio from which p-values are derived

PValue: Associated p-value for this gene's coefficient

FDR: Benjamini-Hochberg False Discover Rate

TABLE 2A
Spleen Ctrl vs KO

| GeneSymbol | logFC | logCPM | LR | PValue | FDR |
|---|---|---|---|---|---|
| B2m | −0.394594818 | 12.75972879 | 1444.404623 | 0 | 0 |
| Rpl8 | 0.468048248 | 11.72273016 | 1161.570028 | 1.3719E−254 | 1.0569E−250 |
| Rpl18 | 0.434763597 | 11.79581946 | 1104.869146 | 2.8881E−242 | 1.4833E−238 |
| Ifi2712a | −1.367469104 | 10.39056913 | 1082.936188 | 1.689E−237 | 6.5061E−234 |
| Rpl14 | 0.467863321 | 11.90179131 | 999.0735997 | 2.8552E−219 | 8.7985E−216 |
| Rpl18a | 0.40844965 | 12.7923771 | 976.338732 | 2.4971E−214 | 6.4125E−211 |
| Rps5 | 0.425613673 | 12.21232007 | 963.9572813 | 1.2269E−211 | 2.7005E−208 |
| Rpl17 | 0.406079885 | 12.23488643 | 922.7043597 | 1.1382E−202 | 2.1922E−199 |
| Tmsb10 | −0.430569001 | 12.90243511 | 917.8571853 | 1.288E−201 | 2.2051E−198 |
| Rpl13a | 0.318130864 | 13.07963904 | 880.2603075 | 1.9189E−193 | 2.9566E−190 |
| Rps6 | 0.387608536 | 12.2862602 | 879.5297959 | 2.766E−193 | 3.8744E−190 |
| Ubb | −0.427975051 | 11.87538618 | 854.4454932 | 7.8543E−188 | 1.0085E−184 |
| Rpl3 | 0.392996062 | 12.07431934 | 853.9654869 | 9.9877E−188 | 1.1838E−184 |
| Rps18 | 0.441028555 | 12.56725217 | 846.7465436 | 3.7058E−186 | 4.0784E−183 |
| Rps3 | 0.326489663 | 11.9717569 | 804.8381021 | 4.7882E−177 | 4.9185E−174 |
| Rps14 | 0.361847774 | 12.58774591 | 790.9068962 | 5.1178E−174 | 4.9284E−171 |
| Rpl29 | 0.403548785 | 11.32743279 | 789.2408217 | 1.1785E−173 | 1.0681E−170 |
| Hbb-bs | −1.322316458 | 9.139899255 | 774.1781884 | 2.2197E−170 | 1.9001E−167 |
| Rps23 | 0.366380189 | 12.42732913 | 772.3794594 | 5.4626E−170 | 4.4298E−167 |
| Rpl32 | 0.342303232 | 12.55404626 | 755.0295723 | 3.2344E−166 | 2.4918E−163 |
| Pfn1 | −0.384163523 | 12.60884857 | 710.1081556 | 1.8951E−156 | 1.3905E−153 |
| Rplp0 | 0.316020006 | 12.54798341 | 706.666425 | 1.0618E−155 | 7.4367E−153 |
| Rpl24 | 0.345910071 | 11.74324764 | 701.1303805 | 1.6978E−154 | 1.1374E−151 |
| Rpl21 | 0.351304057 | 11.78074345 | 696.9180321 | 1.3993E−153 | 8.9833E−151 |
| Rpl13 | 0.383449286 | 12.64381099 | 695.1792673 | 3.342E−153 | 2.0598E−150 |
| Rps24 | 0.387091918 | 11.96746781 | 683.7521651 | 1.0208E−150 | 6.0497E−148 |
| Rpl10a | 0.399442811 | 11.25682159 | 681.0036857 | 4.0426E−150 | 2.307E−147 |
| Il7r | 1.03867114 | 9.716667054 | 678.6930989 | 1.2857E−149 | 7.0749E−147 |
| Rps4x | 0.331978848 | 11.96044745 | 670.9400055 | 6.24E−148 | 3.3154E−145 |
| Rpl19 | 0.284781004 | 12.05752888 | 636.4445613 | 1.9824E−140 | 1.0182E−137 |
| AW112010 | −0.543245425 | 11.59018241 | 633.9186211 | 7.0236E−140 | 3.491E−137 |
| Rpl4 | 0.314340007 | 11.74124626 | 627.5348359 | 1.7178E−138 | 8.2712E−136 |
| Rps19 | 0.395757239 | 12.83023757 | 615.6591388 | 6.5748E−136 | 3.0698E−133 |
| Rps7 | 0.354149685 | 12.00850254 | 606.9322923 | 5.1998E−134 | 2.3564E−131 |
| Rps3a1 | 0.299122173 | 11.79361689 | 588.9227548 | 4.2976E−130 | 1.8919E−127 |
| Rps9 | 0.332982718 | 11.94213912 | 586.8353858 | 1.2225E−129 | 5.2324E−127 |
| Rpl23a | 0.320735899 | 12.0892703 | 578.7802504 | 6.9088E−128 | 2.877E−125 |
| Rpl12 | 0.50132843 | 10.9317396 | 576.0114318 | 2.7649E−127 | 1.1211E−124 |
| Tmsb4x | −0.270263653 | 14.50537537 | 568.0807108 | 1.4683E−125 | 5.8009E−123 |
| Rpl23 | 0.338209227 | 11.6086807 | 556.8680667 | 4.0357E−123 | 1.5546E−120 |
| Rps13 | 0.303056725 | 11.84034228 | 550.8608727 | 8.1793E−122 | 3.0738E−119 |
| mt-Co1 | 0.322058669 | 12.25832103 | 542.7662554 | 4.7167E−120 | 1.7304E−117 |
| Rps10 | 0.322943166 | 11.23934257 | 537.8337752 | 5.5807E−119 | 1.9997E−116 |
| Rsrp1 | −0.547900074 | 10.29205708 | 531.868532 | 1.1077E−117 | 3.8791E−115 |
| Rpl26 | 0.317557283 | 11.55435837 | 530.2211529 | 2.5283E−117 | 8.657E−115 |
| Rps15a | 0.328204873 | 12.2017224 | 529.9491858 | 2.8974E−117 | 9.7049E−115 |
| Rpl6 | 0.299225556 | 11.6914612 | 522.7460832 | 1.0693E−115 | 3.5055E−113 |
| Rpl30 | 0.376508954 | 10.91773473 | 522.2066548 | 1.4011E−115 | 4.4974E−113 |
| Rplp1 | 0.335197804 | 11.71666386 | 510.0333892 | 6.2366E−113 | 1.9611E−110 |
| Rps2 | 0.358100913 | 11.64912595 | 488.7668525 | 2.6432E−108 | 8.1453E−106 |
| Hba-a1 | −0.920500794 | 9.095022031 | 476.6698426 | 1.1334E−105 | 3.4242E−103 |
| Rps8 | 0.286460085 | 11.80270255 | 474.8469891 | 2.8251E−105 | 8.3711E−103 |
| Rpsa | 0.273857677 | 11.6751798 | 471.4739516 | 1.5312E−104 | 4.4514E−102 |
| Rps16 | 0.275293093 | 12.28556335 | 448.530665 | 1.5062E−99 | 4.29771E−97 |
| Rpl36a | 0.442909207 | 10.94274968 | 446.158868 | 4.9438E−99 | 1.38498E−96 |
| Sh3bgrl3 | −0.341561055 | 11.40559323 | 440.3930387 | 8.89009E−98 | 2.44604E−95 |
| Rpl27a | 0.294504688 | 11.64048213 | 438.3535946 | 2.47041E−97 | 6.67791E−95 |
| Cfl1 | −0.314322281 | 11.4854451 | 437.6224597 | 3.56364E−97 | 9.46699E−95 |
| Sub1 | −0.411655331 | 10.76071441 | 436.4866305 | 6.29648E−97 | 1.64434E−94 |
| Myl6 | −0.314779512 | 11.36366325 | 433.2092611 | 3.25386E−96 | 8.35591E−94 |
| Hspa1a | −1.336716152 | 9.242931161 | 430.8805791 | 1.04527E−95 | 2.64026E−93 |
| Rpl34 | 0.278104357 | 11.77163488 | 430.4209651 | 1.31603E−95 | 3.27054E−93 |
| Hnrnpa3 | −0.464034776 | 10.23003684 | 427.6656651 | 5.23551E−95 | 1.28046E−92 |
| Eef2 | 0.294044382 | 11.17784701 | 424.7944526 | 2.20745E−94 | 5.31444E−92 |
| Gnb2l1 | 0.351421332 | 10.53545348 | 417.6969993 | 7.73971E−93 | 1.83467E−90 |
| Prdx1 | −0.443893585 | 10.25016103 | 405.1065332 | 4.25916E−90 | 9.9432E−88 |
| Rps12 | 0.392808532 | 10.82428255 | 404.9917445 | 4.5114E−90 | 1.03749E−87 |
| Eef1b2 | 0.464170287 | 10.11001442 | 404.8440126 | 4.85814E−90 | 1.1008E−87 |
| Rplp2 | 0.281131176 | 11.78981311 | 403.697428 | 8.63098E−90 | 1.92734E−87 |
| Rps27a | 0.272166684 | 11.85785907 | 403.2559785 | 1.07685E−89 | 2.37031E−87 |
| Eef1a1 | 0.230267534 | 12.30506479 | 395.0564055 | 6.56311E−88 | 1.42429E−85 |
| Serf2 | −0.294324233 | 10.78514217 | 392.1550889 | 2.81006E−87 | 6.01352E−85 |
| Rpl11 | 0.246557229 | 11.72200823 | 387.7373487 | 2.57314E−86 | 5.43108E−84 |
| Rps20 | 0.363934892 | 11.13696988 | 381.8405125 | 4.94601E−85 | 1.02984E−82 |
| Rps11 | 0.226974358 | 12.39603371 | 378.4530376 | 2.70247E−84 | 5.55196E−82 |
| Hspa1b | −0.944735319 | 9.137289838 | 356.5410016 | 1.59507E−79 | 3.2338E−77 |
| Rpl5 | 0.359361035 | 10.54545781 | 341.8660149 | 2.50316E−76 | 5.00892E−74 |
| Ms4a4b | −0.306121376 | 11.83323511 | 339.3968419 | 8.63432E−76 | 1.70561E−73 |
| Hmgn2 | −0.9381794 | 9.557710879 | 338.9838185 | 1.06213E−75 | 2.07156E−73 |
| S100a11 | −0.412995978 | 10.68782699 | 337.6388092 | 2.08498E−75 | 4.01566E−73 |
| Cd3e | −0.387729698 | 10.30595328 | 336.0488677 | 4.62777E−75 | 8.80304E−73 |
| Hnrnpa2b1 | −0.40798608 | 10.28844325 | 326.7138354 | 4.99493E−73 | 9.38559E−71 |
| Rps25 | 0.270160344 | 11.11755248 | 325.1522005 | 1.09312E−72 | 2.02925E−70 |
| Rpl36 | 0.292774874 | 11.53238972 | 323.7246145 | 2.23673E−72 | 4.10281E−70 |
| Rpl9 | 0.249983812 | 11.74080817 | 321.4482229 | 7.00566E−72 | 1.26992E−69 |
| Plac8 | 1.012302318 | 9.652571139 | 318.2423485 | 3.49751E−71 | 6.26624E−69 |
| Tsc22d3 | 0.697894281 | 9.554940607 | 317.6897125 | 4.61464E−71 | 8.17268E−69 |
| Myl12a | −0.335034242 | 10.60425227 | 314.7301327 | 2.03621E−70 | 3.56521E−68 |
| Bcl2 | 0.906160685 | 9.588703364 | 313.2636118 | 4.24894E−70 | 7.35592E−68 |
| Hmgb2 | −0.696396429 | 11.06074981 | 311.7640665 | 9.01443E−70 | 1.54327E−67 |
| Stat1 | −0.697258014 | 9.534895283 | 306.4652959 | 1.28603E−68 | 2.17748E−66 |
| Psme2 | −0.393710072 | 10.13966069 | 300.8422195 | 2.15914E−67 | 3.61609E−65 |
| Hba-a2 | −0.676429421 | 9.081900495 | 299.835836 | 3.57715E−67 | 5.92653E−65 |
| Hspa8 | 0.261320173 | 11.52981983 | 289.4131945 | 6.67446E−65 | 1.09404E−62 |
| Tpt1 | 0.204354222 | 11.56490731 | 286.7839208 | 2.49646E−64 | 4.04899E−62 |
| Acp5 | 0.600613336 | 9.490932375 | 273.4792682 | 1.97986E−61 | 3.17768E−59 |
| Ms4a6b | −0.294195147 | 10.88050411 | 272.369232 | 3.45584E−61 | 5.48944E−59 |
| Pdia3 | −0.40435809 | 9.947067745 | 268.7935278 | 2.07901E−60 | 3.26871E−58 |
| Cyba | −0.298957075 | 10.70076607 | 257.3695116 | 6.42556E−58 | 1.00005E−55 |
| Rpl7 | 0.20397207 | 11.24994647 | 255.4080291 | 1.71985E−57 | 2.64994E−55 |
| Dusp2 | 0.695936163 | 9.421733241 | 249.5519128 | 3.25182E−56 | 4.9608E−54 |
| Calm3 | −0.457829973 | 9.738712517 | 249.1451719 | 3.98842E−56 | 6.02486E−54 |
| Gm9844 | −0.598681314 | 9.498462623 | 246.8932384 | 1.23526E−55 | 1.84785E−53 |
| Cdc42 | −0.266127107 | 10.66030324 | 245.2729444 | 2.78624E−55 | 4.12793E−53 |
| Ppia | −0.24330961 | 11.96233228 | 242.3342812 | 1.21825E−54 | 1.7877E−52 |
| Hbb-bt | −0.498398902 | 9.069464739 | 241.8093712 | 1.58558E−54 | 2.30477E−52 |
| Fau | 0.185333561 | 12.0738768 | 241.4373304 | 1.9112E−54 | 2.75213E−52 |
| Rpl7a | 0.267864394 | 10.57501611 | 239.9695494 | 3.99342E−54 | 5.69728E−52 |
| Hsp90b1 | −0.491697038 | 9.746474506 | 236.6017656 | 2.16618E−53 | 3.06206E−51 |
| Eno1 | −0.348932991 | 10.05496627 | 233.3201331 | 1.12538E−52 | 1.57635E−50 |
| Ddit4 | 0.538960929 | 9.672816531 | 233.2496451 | 1.16592E−52 | 1.61843E−50 |
| Ptma | −0.370972377 | 11.21833373 | 223.7026613 | 1.40854E−50 | 1.93775E−48 |
| Ybx1 | −0.285294787 | 10.48744422 | 222.7185656 | 2.30893E−50 | 3.14832E−48 |
| H3f3b | −0.260942477 | 11.14558946 | 221.6453302 | 3.95823E−50 | 5.34986E−48 |
| Itgb1 | −0.666523293 | 10.23396472 | 220.1474805 | 8.39874E−50 | 1.12529E−47 |
| Fxyd5 | 0.269233669 | 10.67479998 | 216.001067 | 6.74044E−49 | 8.95317E−47 |
| Klf2 | 0.339556672 | 11.0136358 | 215.1873446 | 1.01437E−48 | 1.33585E−46 |
| Gm10076 | −0.558406186 | 9.458585833 | 213.9666957 | 1.87276E−48 | 2.44538E−46 |
| Eef1g | 0.293402137 | 10.47224312 | 211.2066869 | 7.49211E−48 | 9.70071E−46 |
| Ifngr1 | 0.49965726 | 9.929229799 | 209.6778484 | 1.61492E−47 | 2.07355E−45 |
| Ly6a | −0.341921357 | 10.6371311 | 209.1069354 | 2.15134E−47 | 2.73949E−45 |
| Tmbim6 | −0.296577287 | 10.12994397 | 206.583007 | 7.64514E−47 | 9.65543E−45 |
| Anxa2 | −0.517046321 | 10.04332864 | 202.0678195 | 7.38922E−46 | 9.25635E−44 |
| Cd52 | −0.210917208 | 12.51348717 | 195.7909614 | 1.73136E−44 | 2.15135E−42 |
| Arl6ip1 | −0.405136227 | 10.09908166 | 195.5723749 | 1.93238E−44 | 2.38192E−42 |
| Ndufa3 | −0.334866237 | 9.839878945 | 195.390331 | 2.11749E−44 | 2.58939E−42 |
| Tuba1b | −0.67879043 | 10.00008493 | 195.1176906 | 2.42843E−44 | 2.94624E−42 |
| Rtp4 | −0.793831448 | 9.147652434 | 194.6736275 | 3.03559E−44 | 3.65409E−42 |
| Capg | −0.495718911 | 9.935583727 | 193.4909519 | 5.50006E−44 | 6.56937E−42 |
| Hnrnpdl | −0.50199538 | 9.362300942 | 193.1180335 | 6.63376E−44 | 7.86254E−42 |
| Tma7 | −0.269138523 | 10.19317796 | 189.7301451 | 3.64112E−43 | 4.28262E−41 |
| Eif3f | 0.233701824 | 10.65753688 | 188.392098 | 7.1336E−43 | 8.32686E−41 |
| Lgals1 | −0.38078271 | 12.63361906 | 187.9718066 | 8.81159E−43 | 1.02082E−40 |
| Rbm3 | 0.277674976 | 10.70166498 | 184.7747377 | 4.39516E−42 | 5.05378E−40 |
| Uba52 | −0.408087039 | 9.952856342 | 183.6984284 | 7.55001E−42 | 8.61708E−40 |
| Sh2d1a | −0.491016053 | 9.642697413 | 179.860533 | 5.19842E−41 | 5.88951E−39 |
| Ctla2a | 0.588010327 | 9.748854778 | 179.6570959 | 5.75825E−41 | 6.47614E−39 |
| Tap1 | −0.415743191 | 9.53747222 | 178.4958846 | 1.03237E−40 | 1.15267E−38 |
| Txn1 | −0.325654396 | 10.05357118 | 176.6338416 | 2.63285E−40 | 2.91849E−38 |
| Arl4c | 0.407528267 | 9.783232926 | 176.3804994 | 2.99053E−40 | 3.29129E−38 |
| Gltscr2 | 0.305069843 | 10.068061 | 174.8774404 | 6.36752E−40 | 6.95821E−38 |
| Ccng1 | −0.542528285 | 9.308847938 | 174.5743604 | 7.41577E−40 | 8.04663E−38 |
| Cox7c | −0.258404342 | 10.16243436 | 174.2395106 | 8.77566E−40 | 9.45563E−38 |
| Sec61g | −0.28454506 | 10.08219258 | 173.0412387 | 1.60311E−39 | 1.71533E−37 |
| Psmb9 | −0.349239421 | 9.782360113 | 172.413577 | 2.19805E−39 | 2.3357E−37 |
| Arhgdib | −0.188477383 | 11.23130777 | 172.327412 | 2.29539E−39 | 2.42242E−37 |
| Rpl35a | 0.170485936 | 11.73666243 | 171.8379009 | 2.93605E−39 | 3.07746E−37 |
| Wdr1 | −0.338161055 | 9.787807742 | 171.5059364 | 3.46947E−39 | 3.612E−37 |
| Itgb2 | −0.319512386 | 10.05832132 | 170.556406 | 5.59298E−39 | 5.78366E−37 |
| Atp5k | −0.362856145 | 9.63851564 | 164.5794993 | 1.13023E−37 | 1.16098E−35 |
| Rabac1 | 0.405039787 | 9.640125814 | 162.7941386 | 2.77456E−37 | 2.83116E−35 |
| Rps27rt | −0.447016904 | 10.76991423 | 162.249778 | 3.64854E−37 | 3.69847E−35 |
| Alyref | −0.535399757 | 9.490162415 | 162.2144125 | 3.71404E−37 | 3.74025E−35 |
| Dtx1 | 0.560691764 | 9.35639362 | 160.7218922 | 7.86911E−37 | 7.8732E−35 |
| Ndufa4 | −0.312282402 | 9.983525258 | 159.2954748 | 1.6128E−36 | 1.60323E−34 |
| Lsm5 | −0.407548255 | 9.652256708 | 159.0796182 | 1.79781E−36 | 1.77568E−34 |
| Il17re | 0.590542136 | 9.074926885 | 158.4513557 | 2.46614E−36 | 2.42028E−34 |
| Tpm3-rs7 | −0.294787222 | 9.951730222 | 158.186127 | 2.8182E−36 | 2.74828E−34 |
| Tapbp | −0.360215199 | 9.633906361 | 158.1391765 | 2.88556E−36 | 2.79627E−34 |
| Rpl27 | 0.199841065 | 11.18362874 | 157.30377 | 4.39311E−36 | 4.23057E−34 |
| Tmem258 | −0.318818743 | 9.706925426 | 157.1801446 | 4.67505E−36 | 4.47411E−34 |
| Btg1 | 0.36057321 | 10.56026433 | 157.00365 | 5.10919E−36 | 4.85941E−34 |
| mt-Co2 | 0.178449904 | 11.53302585 | 156.9703025 | 5.19564E−36 | 4.91132E−34 |
| Tmed2 | −0.296688017 | 9.866059965 | 156.6971734 | 5.96104E−36 | 5.60047E−34 |
| Satb1 | 0.535626973 | 9.41396337 | 154.740485 | 1.59553E−35 | 1.48994E−33 |
| Lcp1 | −0.251556666 | 10.3183034 | 154.3223776 | 1.96914E−35 | 1.82774E−33 |
| Dnajc15 | 0.31387085 | 9.931084321 | 153.721031 | 2.66499E−35 | 2.45881E−33 |
| Cd7 | 0.668431811 | 9.510504639 | 153.5856393 | 2.85288E−35 | 2.6165E−33 |
| Sepw1 | −0.27518717 | 10.11754145 | 150.0979543 | 1.65025E−34 | 1.50456E−32 |
| Ewsr1 | −0.345594062 | 9.627042746 | 150.0365057 | 1.70208E−34 | 1.54269E−32 |
| Cdkn2c | −0.636702077 | 9.207647914 | 149.3358462 | 2.42175E−34 | 2.18213E−32 |
| Actr2 | −0.34076163 | 9.642504656 | 148.8367424 | 3.11334E−34 | 2.78897E−32 |
| Tubb5 | −0.417183951 | 10.30641471 | 145.4558067 | 1.70731E−33 | 1.52059E−31 |
| Ngp | −0.763681094 | 9.100007972 | 145.0011994 | 2.14634E−33 | 1.90062E−31 |
| Isg15 | −0.719651949 | 9.17520338 | 143.8950283 | 3.74577E−33 | 3.29799E−31 |
| Tceb2 | −0.237515156 | 10.20307649 | 142.9938076 | 5.89637E−33 | 5.16201E−31 |
| Rps15 | 0.168454585 | 11.47619806 | 142.2891217 | 8.40744E−33 | 7.31875E−31 |
| Cytip | 0.360334035 | 9.597191973 | 142.1887143 | 8.84338E−33 | 7.65498E−31 |
| Clic1 | −0.218361527 | 10.68495514 | 140.1304999 | 2.49274E−32 | 2.1457E−30 |
| Ube2s | −0.454337114 | 9.814291968 | 137.7313987 | 8.3432E−32 | 7.14178E−30 |
| Klf13 | 0.398593604 | 9.628466206 | 137.2456797 | 1.06552E−31 | 9.07045E−30 |
| Prr13 | −0.278815789 | 10.28885144 | 136.8919515 | 1.27328E−31 | 1.07795E−29 |
| Cdkn2a | −0.609121377 | 9.14054978 | 135.9701759 | 2.0255E−31 | 1.7054E−29 |
| Cox17 | −0.329594045 | 9.66363411 | 135.6492986 | 2.38076E−31 | 1.99363E−29 |
| Stmn1 | −0.82419623 | 9.807967069 | 133.4210017 | 7.31357E−31 | 6.09121E−29 |
| Uqcrb | −0.273218922 | 9.8678254 | 132.0266827 | 1.47622E−30 | 1.22288E−28 |
| Pkm | −0.240731434 | 10.20918593 | 131.7398269 | 1.70571E−30 | 1.40543E−28 |
| 2810417H13Rik | −0.967373055 | 9.637529027 | 131.3105515 | 2.11748E−30 | 1.73543E−28 |
| Rbm39 | 0.23545176 | 10.26100638 | 130.597203 | 3.0331E−30 | 2.4727E−28 |
| Ube2n | −0.31188351 | 9.608778462 | 129.8514637 | 4.41618E−30 | 3.58129E−28 |
| Ywhah | −0.296531841 | 9.906272871 | 129.0537086 | 6.60079E−30 | 5.32486E−28 |
| Calm1 | −0.188744202 | 11.10279906 | 128.1469284 | 1.04234E−29 | 8.36476E−28 |
| Lmnb1 | −0.553332185 | 9.492228417 | 127.3329829 | 1.57079E−29 | 1.25403E−27 |
| Pdia4 | −0.417941212 | 9.32860591 | 126.601842 | 2.27046E−29 | 1.80326E−27 |
| Psma2 | −0.250801241 | 9.885834901 | 126.3445928 | 2.5847E−29 | 2.04231E−27 |
| Ppp4c | −0.306484664 | 9.664046883 | 125.3439349 | 4.27957E−29 | 3.36427E−27 |
| mt-Co3 | 0.148713029 | 12.38729466 | 124.5410801 | 6.41375E−29 | 5.0164E−27 |
| Cox6b1 | −0.201143661 | 10.26082572 | 122.5363554 | 1.76158E−28 | 1.37083E−26 |
| Pomp | −0.26160036 | 9.873253648 | 122.454949 | 1.83536E−28 | 1.42106E−26 |
| Dbi | −0.365045445 | 9.623042963 | 121.9090122 | 2.41671E−28 | 1.86183E−26 |
| Cox7a2l | 0.25365352 | 10.09942318 | 121.8347741 | 2.50885E−28 | 1.91816E−26 |
| 1810037I17Rik | −0.312590156 | 9.676973201 | 121.8301355 | 2.51473E−28 | 1.91816E−26 |
| Zeb2 | −0.774138414 | 9.127424397 | 121.6173644 | 2.79941E−28 | 2.12479E−26 |
| Atp5j | −0.228873779 | 10.1072932 | 121.0463043 | 3.73316E−28 | 2.81963E−26 |
| Npm1 | 0.235150388 | 10.40971878 | 119.8911973 | 6.68273E−28 | 5.0228E−26 |
| Atp1b3 | 0.318801787 | 9.708024434 | 119.2417087 | 9.27148E−28 | 6.93471E−26 |
| Tmem176a | 0.763367915 | 9.182539974 | 119.1449011 | 9.73518E−28 | 7.24636E−26 |
| Pdcd4 | 0.420098355 | 9.642492644 | 118.9155041 | 1.09287E−27 | 8.09565E−26 |
| Cd247 | −0.333565058 | 9.579185183 | 118.7057752 | 1.21475E−27 | 8.95546E−26 |
| Rpl22l1 | 0.208609039 | 10.83580793 | 118.6842487 | 1.22801E−27 | 9.01007E−26 |
| Hnrnpa1 | −0.369311881 | 9.492414828 | 118.3490179 | 1.45412E−27 | 1.06185E−25 |
| Shfm1 | −0.229553147 | 10.0031327 | 117.0924606 | 2.73992E−27 | 1.99135E−25 |
| Rps21 | 0.238376482 | 10.63600103 | 116.6726207 | 3.38588E−27 | 2.44928E−25 |
| Tceb1 | −0.31869581 | 9.529590194 | 116.660403 | 3.40681E−27 | 2.4529E−25 |
| Srrm2 | 0.30189204 | 9.744719687 | 116.4576604 | 3.7735E−27 | 2.70428E−25 |
| Canx | −0.374606446 | 9.405124767 | 116.1573065 | 4.39053E−27 | 3.13191E−25 |
| Cenpa | −0.706828827 | 9.37944655 | 115.835444 | 5.16417E−27 | 3.6668E−25 |
| Lztfl1 | 0.577921378 | 9.128139284 | 115.7740802 | 5.32646E−27 | 3.76468E−25 |
| Pglyrp1 | −0.371039398 | 9.700031151 | 114.7295603 | 9.01963E−27 | 6.34587E−25 |
| Ifi27 | −0.367396341 | 9.485380503 | 114.4243909 | 1.05202E−26 | 7.36797E−25 |
| Cox6c | −0.199529709 | 10.2464669 | 114.0849521 | 1.24844E−26 | 8.70403E−25 |
| Myl12b | −0.219178436 | 10.11053816 | 113.7928401 | 1.44659E−26 | 1.00401E−24 |
| Igfbp4 | 0.750607125 | 9.125936959 | 113.3553129 | 1.80375E−26 | 1.24628E−24 |
| S100a9 | −0.788616203 | 9.108492579 | 112.2912902 | 3.08488E−26 | 2.12196E−24 |
| Nkg7 | −0.352273992 | 11.35660318 | 112.1715336 | 3.27696E−26 | 2.24406E−24 |
| Wsb1 | −0.434373342 | 9.210910529 | 110.9196712 | 6.16175E−26 | 4.2009E−24 |
| Cox7a2 | −0.244165019 | 9.904372175 | 110.9093932 | 6.19378E−26 | 4.20413E−24 |
| Erh | −0.267511865 | 9.871191334 | 110.0778456 | 9.42172E−26 | 6.3671E−24 |
| Cks1b | −0.63195329 | 9.374265449 | 109.9305553 | 1.01484E−25 | 6.82826E−24 |
| Ube2l6 | −0.474836411 | 9.105066783 | 108.9541719 | 1.66082E−25 | 1.1126E−23 |
| Capza1 | −0.321589878 | 9.49698091 | 108.9198525 | 1.68982E−25 | 1.12713E−23 |
| Pik3ip1 | 0.517288658 | 9.141032834 | 108.454519 | 2.13698E−25 | 1.41925E−23 |
| S100a10 | −0.239806506 | 11.56390824 | 107.633748 | 3.23332E−25 | 2.13816E−23 |
| Cd160 | 0.699025169 | 9.159417004 | 107.4577704 | 3.53355E−25 | 2.32671E−23 |
| Rbx1 | −0.227759516 | 9.906193708 | 107.2191678 | 3.98563E−25 | 2.61322E−23 |
| Gzma | 1.120093335 | 10.5303845 | 106.7164441 | 5.13647E−25 | 3.3535E−23 |
| Camp | −0.466568955 | 9.07752253 | 106.5101125 | 5.70009E−25 | 3.70578E−23 |
| Ctsb | −0.318746012 | 9.577536052 | 106.4526506 | 5.86779E−25 | 3.79878E−23 |
| Vcp | −0.290019259 | 9.600777235 | 105.6169585 | 8.94588E−25 | 5.76729E−23 |
| Ppp1ca | −0.179862972 | 10.60111574 | 105.4922694 | 9.52689E−25 | 6.11626E−23 |
| Ran | −0.290507861 | 10.03079907 | 105.213453 | 1.09663E−24 | 6.98217E−23 |
| Zbp1 | −0.425278438 | 9.318275709 | 105.2157644 | 1.09535E−24 | 6.98217E−23 |
| H2-D1 | −0.124251699 | 12.15670663 | 104.0015903 | 2.02154E−24 | 1.2818E−22 |
| Myc | 0.512412291 | 9.116237101 | 103.4698379 | 2.64389E−24 | 1.66955E−22 |
| Atp8a2 | 0.33886315 | 9.069620419 | 101.2967204 | 7.91868E−24 | 4.98004E−22 |
| Use1 | 0.333960173 | 9.51419359 | 100.7604386 | 1.03809E−23 | 6.502E−22 |
| Iigp1 | −0.614742376 | 9.132296579 | 100.6538941 | 1.09546E−23 | 6.83355E−22 |
| Eif4g2 | −0.258455174 | 9.738725432 | 100.5005074 | 1.18367E−23 | 7.354E−22 |
| Cdk1 | −0.677159182 | 9.209723077 | 100.3484505 | 1.27812E−23 | 7.90893E−22 |
| Pttg1 | −0.428228606 | 9.427760824 | 100.0562925 | 1.48127E−23 | 9.12934E−22 |
| Tomm7 | −0.264886375 | 9.683676151 | 99.52716681 | 1.93491E−23 | 1.18777E−21 |
| Ubl5 | −0.217789014 | 9.954866528 | 98.70357382 | 2.93272E−23 | 1.79315E−21 |
| Gnb1 | −0.310852241 | 9.598111237 | 98.56239704 | 3.14943E−23 | 1.91804E−21 |
| Hnrnpl | −0.318458149 | 9.458965323 | 98.41828059 | 3.38718E−23 | 2.05471E−21 |
| Tecpr1 | 0.380153257 | 9.37320746 | 98.26258599 | 3.66424E−23 | 2.21406E−21 |
| Hprt | −0.296022775 | 9.583675978 | 97.63539867 | 5.02967E−23 | 3.02723E−21 |
| Tmem176b | 0.699466756 | 9.197393426 | 97.44004885 | 5.55118E−23 | 3.32812E−21 |
| Ywhaq | −0.251767896 | 9.876197724 | 97.04439604 | 6.77901E−23 | 4.04849E−21 |
| Tmem234 | 0.291933521 | 9.600002857 | 96.25813041 | 1.0084E−22 | 5.99902E−21 |
| Cd3g | −0.157389506 | 10.99201317 | 95.96100216 | 1.17169E−22 | 6.94361E−21 |
| Arpc5 | −0.208198677 | 10.28464975 | 95.71542866 | 1.32643E−22 | 7.83049E−21 |
| Lockd | −0.440867625 | 9.277369705 | 95.06964861 | 1.83803E−22 | 1.08093E−20 |
| Ctsd | −0.266169224 | 10.13340333 | 94.71984408 | 2.19329E−22 | 1.28495E−20 |
| Atp5j2 | −0.19242437 | 10.13808543 | 94.60218126 | 2.32762E−22 | 1.35848E−20 |
| Pyhin1 | −0.418379849 | 9.287028936 | 92.6509167 | 6.23817E−22 | 3.62708E−20 |
| Tox | −0.508928639 | 9.127330992 | 92.52335084 | 6.65351E−22 | 3.85403E−20 |
| Gnai2 | −0.206265604 | 10.42766401 | 92.48466885 | 6.78484E−22 | 3.91538E−20 |
| ler2 | 0.319611087 | 9.601826126 | 92.17549933 | 7.93207E−22 | 4.56035E−20 |
| Erdr1 | 0.464102541 | 9.15671915 | 91.25689209 | 1.2618E−21 | 7.22743E−20 |
| Polr2k | −0.3265931 | 9.353097422 | 91.23516042 | 1.27573E−21 | 7.28017E−20 |
| Hspa5 | −0.281941206 | 9.834386077 | 90.86640181 | 1.53708E−21 | 8.73921E−20 |
| Calr | −0.270251962 | 9.906445352 | 90.85450983 | 1.54634E−21 | 8.75957E−20 |
| Hnrnpc | −0.276698646 | 9.578766056 | 90.02748793 | 2.34874E−21 | 1.32562E−19 |
| Ccnb1 | −0.644827058 | 9.19263149 | 88.81860324 | 4.32727E−21 | 2.43338E−19 |
| Ubc | −0.215315232 | 10.26377247 | 88.71348916 | 4.56343E−21 | 2.55685E−19 |
| Gltp | −0.291647317 | 9.558026501 | 88.50731529 | 5.06472E−21 | 2.82744E−19 |
| Lyz2 | −0.428623551 | 9.078273475 | 88.19924611 | 5.91825E−21 | 3.292E−19 |
| Usmg5 | −0.24683068 | 9.690988814 | 87.30309401 | 9.31021E−21 | 5.16014E−19 |
| Top2a | −0.71236757 | 9.359267312 | 86.94889885 | 1.11362E−20 | 6.15004E−19 |
| Prc1 | −0.564899799 | 9.171688394 | 86.75767354 | 1.22667E−20 | 6.75019E−19 |
| Mcm4 | −0.46256567 | 9.182537346 | 86.62660008 | 1.31072E−20 | 7.18705E−19 |
| Fkbp5 | 0.406142801 | 9.186149988 | 86.58136614 | 1.34105E−20 | 7.32726E−19 |
| Nebl | 0.450513843 | 9.267107621 | 86.56702713 | 1.35081E−20 | 7.3545E−19 |
| Ube2c | −0.742287675 | 9.351481647 | 86.45041123 | 1.43285E−20 | 7.77374E−19 |
| Serinc3 | −0.415468602 | 9.341672534 | 86.3641674 | 1.49672E−20 | 8.06347E−19 |
| Zfos1 | 0.384189119 | 9.321085288 | 86.37049565 | 1.49194E−20 | 8.06347E−19 |
| Atp5b | −0.177130163 | 10.37817024 | 86.3041995 | 1.54281E−20 | 8.28277E−19 |
| Hn1 | −0.246470949 | 9.902695291 | 85.15461992 | 2.75922E−20 | 1.47618E−18 |
| Sumo2 | −0.192747248 | 10.32612922 | 85.14414532 | 2.77388E−20 | 1.47889E−18 |
| Kpna2 | −0.504832734 | 9.174376154 | 84.76049621 | 3.36786E−20 | 1.78938E−18 |
| Nrp1 | −0.485433003 | 9.201842589 | 84.64158269 | 3.57663E−20 | 1.89377E−18 |
| Cd48 | −0.2378112 | 10.47381146 | 84.55076715 | 3.74475E−20 | 1.976E−18 |
| Sec11c | −0.261864887 | 9.668953903 | 84.28019512 | 4.29395E−20 | 2.25806E−18 |
| Psma1 | −0.24204948 | 9.631502029 | 84.05535049 | 4.81115E−20 | 2.52143E−18 |
| Pdia6 | −0.351868353 | 9.419021935 | 83.95203204 | 5.06926E−20 | 2.6477E−18 |
| Il2rg | −0.24963658 | 9.757825523 | 83.84407232 | 5.35379E−20 | 2.78686E−18 |
| Gstp1 | −0.388378062 | 9.150648089 | 83.63583556 | 5.94849E−20 | 3.08601E−18 |
| Phf11b | −0.316529852 | 9.423111324 | 83.42271577 | 6.62563E−20 | 3.42576E−18 |
| Lsp1 | −0.184489544 | 10.86029924 | 83.39875856 | 6.70641E−20 | 3.45593E−18 |
| Eif1 | −0.129281955 | 11.09222913 | 83.30726216 | 7.02411E−20 | 3.60758E−18 |
| Cd82 | −0.251979593 | 9.997069085 | 83.03896084 | 8.0452E−20 | 4.11829E−18 |
| Cox6a1 | −0.18589785 | 10.09965413 | 82.98347132 | 8.27424E−20 | 4.2215E−18 |
| Mcm6 | −0.434741928 | 9.318941361 | 82.92037214 | 8.54262E−20 | 4.34405E−18 |
| Klrg1 | −0.66112222 | 9.201832156 | 82.30022292 | 1.16909E−19 | 5.92544E−18 |
| Tpx2 | −0.545063238 | 9.154537098 | 82.14046294 | 1.26751E−19 | 6.40321E−18 |
| Gm4955 | −0.422636634 | 9.199318312 | 82.10881749 | 1.28797E−19 | 6.48529E−18 |
| Birc5 | −0.689978042 | 9.419044284 | 82.03479723 | 1.33712E−19 | 6.71085E−18 |
| Bcl2a1d | −0.405058381 | 9.237077308 | 81.62305429 | 1.64682E−19 | 8.21171E−18 |
| Arpc3 | −0.180051392 | 10.13466789 | 81.62580935 | 1.64452E−19 | 8.21171E−18 |
| Pim1 | 0.390466534 | 9.465218659 | 80.9601044 | 2.30321E−19 | 1.14477E−17 |
| Ly6c1 | 0.593973796 | 9.127161031 | 80.76900017 | 2.53707E−19 | 1.25695E−17 |
| Irf9 | −0.394471258 | 9.19085953 | 80.46333485 | 2.96149E−19 | 1.46252E−17 |
| H2-Ke6 | 0.382623802 | 9.247819067 | 80.1878396 | 3.40456E−19 | 1.67596E−17 |
| Rrm2 | −0.775593179 | 9.444956821 | 80.1075085 | 3.54582E−19 | 1.73994E−17 |
| Gm12840 | 0.522105321 | 9.292404221 | 80.07311968 | 3.60808E−19 | 1.76486E−17 |
| Reep5 | −0.24357654 | 9.869875747 | 80.00448864 | 3.7356E−19 | 1.82146E−17 |
| Ghitm | −0.274937749 | 9.459227183 | 79.78746135 | 4.1693E−19 | 2.02652E−17 |
| Ltf | −0.187637988 | 9.059775685 | 79.75490723 | 4.23857E−19 | 2.05371E−17 |
| Gbp4 | −0.389063896 | 9.207766155 | 79.50041075 | 4.82125E−19 | 2.32871E−17 |
| Glrx | −0.340129563 | 9.454384182 | 79.40690341 | 5.05492E−19 | 2.43394E−17 |
| Kif20a | −0.451598251 | 9.112260701 | 79.35667874 | 5.18507E−19 | 2.48883E−17 |
| Psmb2 | −0.226426476 | 9.718811454 | 79.29191389 | 5.35785E−19 | 2.56378E−17 |
| mt-Atp6 | 0.121761131 | 12.70239722 | 78.91658426 | 6.47886E−19 | 3.0906E−17 |
| Smc4 | −0.3872419 | 9.525409897 | 78.88407307 | 6.58636E−19 | 3.13218E−17 |
| Gpr183 | 0.431381035 | 9.245014018 | 78.76805609 | 6.98474E−19 | 3.31141E−17 |
| Ifit1 | −0.619753615 | 9.092845233 | 78.68290656 | 7.29238E−19 | 3.44666E−17 |
| Mad2l1 | −0.454685107 | 9.179564856 | 78.45025661 | 8.20383E−19 | 3.86558E−17 |
| Ccna2 | −0.640701651 | 9.236667297 | 78.27864952 | 8.94838E−19 | 4.20356E−17 |
| Tacc3 | −0.502848596 | 9.173056684 | 77.6145441 | 1.25244E−18 | 5.86555E−17 |
| Hnrnpk | −0.164547584 | 10.2842712 | 77.29445282 | 1.47279E−18 | 6.8766E−17 |
| Klrc1 | −0.449082904 | 9.467992396 | 77.21304596 | 1.53476E−18 | 7.1443E−17 |
| H2afx | −0.539317748 | 9.440262415 | 76.90569528 | 1.79319E−18 | 8.32214E−17 |
| Lcn2 | −0.206390753 | 9.061453383 | 76.82683074 | 1.86625E−18 | 8.63518E−17 |
| Rpa3 | −0.380997971 | 9.230128239 | 76.68099999 | 2.00927E−18 | 9.26912E−17 |
| Rpl35 | −0.158961239 | 11.13831905 | 76.53702308 | 2.16123E−18 | 9.94037E−17 |
| Fkbp2 | −0.362439923 | 9.306762226 | 76.51376299 | 2.18684E−18 | 1.00282E−16 |
| Slc25a5 | 0.166065043 | 10.50386566 | 76.50621506 | 2.19521E−18 | 1.00367E−16 |
| Cks2 | −0.49697529 | 9.402047794 | 76.41366059 | 2.30054E−18 | 1.04872E−16 |
| Atp5e | −0.167024801 | 10.21622649 | 76.28704667 | 2.45288E−18 | 1.11486E−16 |
| Banf1 | −0.308953202 | 9.400476146 | 75.55734849 | 3.54946E−18 | 1.60853E−16 |
| Emb | 0.269409659 | 9.697311771 | 75.23259584 | 4.18402E−18 | 1.89054E−16 |
| Gbp2 | −0.415666499 | 9.261598091 | 74.89491319 | 4.96447E−18 | 2.23663E−16 |
| Cenpe | −0.487374968 | 9.137306059 | 74.4729421 | 6.14751E−18 | 2.76154E−16 |
| Nusap1 | −0.72741448 | 9.229804121 | 74.10995709 | 7.38847E−18 | 3.30935E−16 |
| 0610010K14Rik | −0.344310005 | 9.213495182 | 73.8659897 | 8.36044E−18 | 3.73385E−16 |
| Cdc20 | −0.647281342 | 9.224041274 | 73.85642712 | 8.40104E−18 | 3.74113E−16 |
| Capza2 | −0.218860051 | 9.723851677 | 73.74804301 | 8.87522E−18 | 3.94091E−16 |
| Gramd3 | 0.340204113 | 9.380450868 | 73.42778822 | 1.04386E−17 | 4.62178E−16 |
| Sept7 | −0.235753818 | 9.711512245 | 73.25364665 | 1.14014E−17 | 5.03362E−16 |
| Actn2 | 0.525915728 | 9.209592958 | 73.0212856 | 1.28259E−17 | 5.64633E−16 |
| Fbxo5 | −0.558938262 | 9.177037636 | 72.85443826 | 1.39573E−17 | 6.12691E−16 |
| Kif22 | −0.493813608 | 9.15961124 | 72.47963983 | 1.68764E−17 | 7.38727E−16 |
| Xaf1 | −0.393829891 | 9.158800712 | 72.38895324 | 1.767E−17 | 7.71274E−16 |
| 2700094K13Rik | −0.315350601 | 9.485438786 | 71.71621114 | 2.48483E−17 | 1.08153E−15 |
| Ranbp1 | −0.272803989 | 9.7032701 | 71.66786291 | 2.54646E−17 | 1.10524E−15 |
| Ssbp2 | 0.400331997 | 9.148764243 | 71.61265527 | 2.61872E−17 | 1.1334E−15 |
| S100a8 | −0.942163941 | 9.144819806 | 71.51122989 | 2.75685E−17 | 1.18985E−15 |
| Rbbp7 | −0.324552015 | 9.3940054 | 71.46912886 | 2.8163E−17 | 1.21211E−15 |
| Rpl22 | 0.157903002 | 10.51485597 | 71.45778022 | 2.83255E−17 | 1.21571E−15 |
| Rbbp4 | −0.279482571 | 9.494526185 | 71.18615873 | 3.25061E−17 | 1.39126E−15 |
| Emp3 | −0.193585605 | 10.48526769 | 71.03770835 | 3.50462E−17 | 1.49582E−15 |
| Rpl31 | 0.137375726 | 10.8998412 | 70.82973716 | 3.89423E−17 | 1.65752E−15 |
| Dek | −0.326257475 | 9.575932497 | 70.56532173 | 4.45276E−17 | 1.89003E−15 |
| Pdcd1 | −0.412579078 | 9.161776078 | 70.07124061 | 5.72009E−17 | 2.42129E−15 |
| Pde2a | 0.378065671 | 9.096741072 | 69.85305389 | 6.38911E−17 | 2.69708E−15 |
| Timp2 | 0.426434752 | 9.155914851 | 69.76664138 | 6.67523E−17 | 2.81016E−15 |
| Car5b | −0.401522657 | 9.137375624 | 69.60125161 | 7.25908E−17 | 3.04763E−15 |
| Spc25 | −0.494263147 | 9.140489027 | 69.5855285 | 7.31718E−17 | 3.06367E−15 |
| Nap1l1 | −0.297646823 | 9.452697682 | 69.31010443 | 8.41374E−17 | 3.51325E−15 |
| Ifng | −0.470155564 | 9.195873407 | 69.27531811 | 8.56346E−17 | 3.5661E−15 |
| Plscr1 | −0.366671337 | 9.165831717 | 69.15277185 | 9.11242E−17 | 3.78448E−15 |
| Ncapg | −0.441222811 | 9.11714452 | 69.07600109 | 9.47412E−17 | 3.92412E−15 |
| Capzb | −0.165768529 | 10.32096611 | 68.88018602 | 1.04631E−16 | 4.32212E−15 |
| Mrps21 | −0.244713874 | 9.534978075 | 68.45138093 | 1.30045E−16 | 5.35756E−15 |
| Ccnb2 | −0.590482352 | 9.326857307 | 68.23617077 | 1.45041E−16 | 5.95943E−15 |
| Rpl39 | 0.126575039 | 11.54192101 | 68.06484771 | 1.58206E−16 | 6.48308E−15 |

TABLE 2B
Colon LPL Ctrl vs KO

| GeneSymbol | logFC | logCPM | LR | PValue | FDR |
|---|---|---|---|---|---|
| Hsp90aa1 | −1.296438691 | 10.855082 | 1796.189057 | 0 | 0 |
| Rpl35 | 0.579539672 | 11.07230469 | 1743.711997 | 0 | 0 |
| Dnaja1 | −1.10964182 | 10.81123241 | 1596.155309 | 0 | 0 |
| Dnajb1 | −2.33531776 | 10.34134508 | 2179.594615 | 0 | 0 |
| Hsp90ab1 | −0.982797393 | 11.27051881 | 2052.383487 | 0 | 0 |
| Ubb | −0.694803098 | 12.95887764 | 2028.318147 | 0 | 0 |
| Hspa8 | −0.737680613 | 12.41996892 | 1606.669603 | 0 | 0 |
| Hspa1a | −3.015660998 | 11.06978681 | 1470.85346 | 0 | 0 |
| Gm8730 | 0.693343323 | 10.36024832 | 1323.369049 | 9.437E−290 | 1.6608E−286 |
| Malat1 | −0.593417954 | 14.67213889 | 1137.684854 | 2.1302E−249 | 3.374E−246 |
| Hspe1 | −1.038624464 | 10.30562386 | 1074.381316 | 1.2219E−235 | 1.7594E−232 |
| Rbm3 | 0.546053782 | 10.66488443 | 928.3699119 | 6.678E−204 | 8.8144E−201 |
| Gm10076 | 0.869179815 | 9.517410456 | 890.0933345 | 1.3977E−195 | 1.703E−192 |
| Chchd2 | −0.492618776 | 10.4561239 | 823.1952025 | 4.8876E−181 | 5.5296E−178 |
| Hsph1 | −1.267762622 | 9.425286154 | 757.4238333 | 9.7545E−167 | 1.03E−163 |
| Jund | −0.668130275 | 11.8228244 | 748.5293847 | 8.3785E−165 | 8.2942E−162 |
| Rplp0 | 0.268693712 | 12.37075798 | 671.3237314 | 5.1491E−148 | 4.7975E−145 |
| Hspa1b | −1.599727007 | 9.248134998 | 620.7646715 | 5.0985E−137 | 4.4864E−134 |
| Ddx5 | −0.386151289 | 11.1817586 | 603.7799595 | 2.5213E−133 | 2.1019E−130 |
| H2-D1 | −0.247894687 | 12.34520231 | 546.2254928 | 8.3388E−121 | 6.6039E−118 |
| H2-K1 | −0.323864629 | 11.66121845 | 544.5044601 | 1.9747E−120 | 1.4894E−117 |
| Hspd1 | −0.837182162 | 9.767644293 | 537.1408453 | 7.8966E−119 | 5.6852E−116 |
| Fosb | −1.164635491 | 9.364618516 | 517.7187705 | 1.327E−114 | 9.1381E−112 |
| H2-Q7 | −0.422171081 | 10.64378871 | 516.4435666 | 2.5136E−114 | 1.6589E−111 |
| Eef2 | 0.274087183 | 11.28374076 | 505.1485441 | 7.2071E−112 | 4.5661E−109 |
| mt-Co1 | 0.272956043 | 12.17896251 | 503.0154954 | 2.0983E−111 | 1.2783E−108 |
| Eno1 | 0.551566329 | 9.902385531 | 499.021854 | 1.5517E−110 | 9.1025E−108 |
| Rpl32 | 0.22194434 | 12.42741546 | 492.9658819 | 3.2246E−109 | 1.8241E−106 |
| Rgcc | −1.118136609 | 9.550274028 | 486.3554888 | 8.8475E−108 | 4.8323E−105 |
| Rpl6l | 0.478179424 | 10.15590832 | 484.5969007 | 2.1354E−107 | 1.1274E−104 |
| Fth1 | −0.335462508 | 11.63346883 | 472.0661866 | 1.138E−104 | 5.8146E−102 |
| B2m | −0.19628426 | 12.80490735 | 466.5540764 | 1.8015E−103 | 8.9168E−101 |
| Rpl29 | 0.283720025 | 11.21054861 | 459.7879827 | 5.346E−102 | 2.5659E−99 |
| Arhgdib | 0.386013595 | 10.67695562 | 447.048832 | 3.165E−99 | 1.47443E−96 |
| Lmna | −0.94001507 | 9.757618312 | 441.2087912 | 5.90701E−98 | 2.67317E−95 |
| Crem | −0.759239846 | 10.12298761 | 430.3980194 | 1.33125E−95 | 5.85713E−93 |
| Ubald2 | −0.477416083 | 10.61574622 | 420.040934 | 2.39076E−93 | 1.02344E−90 |
| Eif1 | −0.24664365 | 11.86551627 | 418.9616487 | 4.10635E−93 | 1.71159E−90 |
| Odc1 | −0.632215319 | 10.52031093 | 405.1128919 | 4.24561E−90 | 1.72426E−87 |
| Rpl3 | 0.197968255 | 12.08615037 | 391.114481 | 4.73428E−87 | 1.87466E−84 |
| Gm2000 | 0.620129441 | 9.580454828 | 389.15096 | 1.26682E−86 | 4.89394E−84 |
| Gm11808 | 0.472407768 | 10.12797784 | 386.6017314 | 4.54666E−86 | 1.71463E−83 |
| Hspa5 | −0.400182424 | 11.01753787 | 383.96611 | 1.70407E−85 | 6.27694E−83 |
| Coro1a | 0.372189609 | 10.69909524 | 376.690267 | 6.53959E−84 | 2.3541E−81 |
| Prr7 | −0.593803345 | 9.822329769 | 370.3859824 | 1.54225E−82 | 5.42837E−80 |
| Rps11 | 0.197413243 | 12.24071835 | 370.1436999 | 1.74143E−82 | 5.9962E−80 |
| Limd2 | 0.478902583 | 9.998680235 | 369.6083445 | 2.27756E−82 | 7.67538E−80 |
| Bin2 | 0.616456928 | 9.522717136 | 361.9853895 | 1.04058E−80 | 3.43369E−78 |
| Rpl22l1 | 0.33367178 | 10.80916121 | 354.53505 | 4.361E−79 | 1.40967E−76 |
| Eif2s3y | 0.846852212 | 9.189708727 | 353.9086731 | 5.97013E−79 | 1.89122E−76 |
| Cd52 | 0.329531615 | 11.76968202 | 350.1895093 | 3.85364E−78 | 1.19682E−75 |
| Gm9493 | 0.397130464 | 10.27648549 | 349.5148795 | 5.40483E−78 | 1.64629E−75 |
| Cdkn1a | −0.794410975 | 9.655752351 | 348.30647 | 9.90677E−78 | 2.96063E−75 |
| Junb | −0.439739713 | 11.78308969 | 344.4692692 | 6.78518E−77 | 1.9902E−74 |
| Rpl27-ps3 | 0.564371664 | 9.812969165 | 341.4961833 | 3.01322E−76 | 8.67752E−74 |
| Slc25a5 | 0.356515832 | 10.33629693 | 334.2365652 | 1.14835E−74 | 3.24799E−72 |
| Klf2 | 0.966338109 | 9.87646467 | 331.2250796 | 5.19954E−74 | 1.44483E−71 |
| Rac2 | 0.275882336 | 11.06163595 | 325.2530092 | 1.03923E−72 | 2.83799E−70 |
| Rpl15 | 0.276369843 | 10.68387878 | 321.477891 | 6.90219E−72 | 1.85295E−69 |
| Gm9844 | 0.5647633 | 9.461659088 | 318.7422393 | 2.72189E−71 | 7.18533E−69 |
| Ptprcap | 0.476661033 | 9.942632577 | 318.6711031 | 2.82076E−71 | 7.32426E−69 |
| Rpl24 | 0.194887307 | 11.7645521 | 317.3192251 | 5.55699E−71 | 1.41963E−68 |
| Fus | −0.428773907 | 9.866010533 | 316.630995 | 7.84794E−71 | 1.97307E−68 |
| Rpl13-ps3 | 0.525225173 | 9.582684681 | 312.3660701 | 6.66497E−70 | 1.64948E−67 |
| S100a6 | 0.525962148 | 11.03347261 | 310.3419701 | 1.83962E−69 | 4.48274E−67 |
| Eif3f | 0.27112225 | 10.61842693 | 309.6198908 | 2.64259E−69 | 6.34181E−67 |
| H2-Q6 | −0.488240508 | 9.705178604 | 307.3949645 | 8.06718E−69 | 1.90711E−66 |
| Eif5 | −0.462609431 | 9.797249973 | 305.4905297 | 2.09703E−68 | 4.88455E−66 |
| Apobec3 | 0.625969666 | 9.401539187 | 304.9819365 | 2.70648E−68 | 6.21274E−66 |
| Gm9843 | 0.405097402 | 10.47924093 | 303.2731905 | 6.37773E−68 | 1.4431E−65 |
| H1f0 | 0.85409956 | 9.356160563 | 303.2280563 | 6.52377E−68 | 1.45535E−65 |
| Cnn2 | 0.459644323 | 9.811452628 | 296.4205581 | 1.98439E−66 | 4.36538E−64 |
| Sertad1 | −0.717279998 | 9.235302519 | 293.8116324 | 7.34606E−66 | 1.59389E−63 |
| Lsp1 | 0.381730916 | 10.25962615 | 291.7842784 | 2.03134E−65 | 4.34789E−63 |
| Rpl9-ps6 | 0.500123891 | 9.813279691 | 290.7553132 | 3.40393E−65 | 7.18865E−63 |
| Rpl10-ps3 | 0.553256709 | 9.514158932 | 289.0389389 | 8.05312E−65 | 1.67833E−62 |
| Ubc | −0.375514664 | 10.9972925 | 286.6902384 | 2.6166E−64 | 5.38238E−62 |
| Zfp3612 | 0.599554856 | 9.691467426 | 281.0413259 | 4.45323E−63 | 9.0429E−61 |
| Pycard | 0.595529185 | 9.493681974 | 279.3579798 | 1.03634E−62 | 2.0778E−60 |
| Gm10073 | 0.522116351 | 9.637520171 | 278.0697639 | 1.97803E−62 | 3.91625E−60 |
| Rgs2 | −0.635850555 | 10.09899367 | 276.0893809 | 5.34329E−62 | 1.04484E−59 |
| Rpl23a-ps3 | 0.343715493 | 10.69786568 | 274.1111792 | 1.44185E−61 | 2.78506E−59 |
| S100a4 | 0.738831043 | 9.845301206 | 271.3430121 | 5.78373E−61 | 1.10372E−58 |
| Epsti1 | 0.443939453 | 9.867770305 | 266.4193722 | 6.84401E−60 | 1.2905E−57 |
| Ifrd1 | −0.474596587 | 10.00161652 | 262.3022178 | 5.40376E−59 | 1.00694E−56 |
| Cacybp | −0.560229675 | 9.442844244 | 260.8502655 | 1.1199E−58 | 2.06257E−56 |
| Uba52 | 0.356717075 | 10.18138302 | 260.7323335 | 1.18819E−58 | 2.16319E−56 |
| Arf4 | −0.398360184 | 9.945640276 | 259.6927564 | 2.00211E−58 | 3.60357E−56 |
| Mat2a | −0.579951827 | 9.446706214 | 253.6395059 | 4.17847E−57 | 7.43626E−55 |
| Cd7 | 0.9863336 | 9.31700291 | 253.534729 | 4.40411E−57 | 7.75074E−55 |
| Lcp1 | 0.350365569 | 10.21732737 | 253.1792183 | 5.26452E−57 | 9.16315E−55 |
| Gm10020 | 0.626059668 | 9.179717204 | 250.3467675 | 2.18193E−56 | 3.75648E−54 |
| Saraf | −0.367375045 | 10.20218079 | 248.9026846 | 4.50471E−56 | 7.67205E−54 |
| Pglyrp1 | 0.745685753 | 9.248613864 | 248.7407525 | 4.88618E−56 | 8.23322E−54 |
| Hmha1 | 0.4442368 | 9.65919282 | 245.5542415 | 2.4193E−55 | 4.03361E−53 |
| Tbc1d10c | 0.555749736 | 9.412018375 | 245.2494518 | 2.8193E−55 | 4.65155E−53 |
| Il21r | −0.437344694 | 10.2017939 | 235.2828562 | 4.20044E−53 | 6.85885E−51 |
| Got1 | −0.578693665 | 9.873061625 | 227.775993 | 1.82126E−51 | 2.94356E−49 |
| Id2 | −0.428153652 | 10.67713064 | 227.1293055 | 2.52005E−51 | 4.03183E−49 |
| Gzma | 1.097864847 | 10.4314706 | 224.6382402 | 8.80465E−51 | 1.39457E−48 |
| Ass1 | 0.551203488 | 9.461406247 | 224.0954108 | 1.1564E−50 | 1.81348E−48 |
| Atf3 | −0.847490224 | 9.233159259 | 220.5128652 | 6.99061E−50 | 1.08553E−47 |
| Gnai2 | 0.343784913 | 10.10321215 | 218.455168 | 1.96497E−49 | 3.02167E−47 |
| Vgll4 | −0.495246625 | 9.708628848 | 217.328475 | 3.46041E−49 | 5.21994E−47 |
| Gm10036 | 0.488692316 | 9.447957927 | 217.3445053 | 3.43266E−49 | 5.21994E−47 |
| Vps37b | −0.356521425 | 10.62817923 | 213.0553128 | 2.96009E−48 | 4.42311E−46 |
| Ctla4 | −0.425992423 | 10.1474776 | 212.5306436 | 3.85271E−48 | 5.70309E−46 |
| Pfn1 | 0.240155328 | 12.08589684 | 211.5092731 | 6.43562E−48 | 9.43832E−46 |
| Gm6133 | 0.385457869 | 9.838579615 | 210.5516257 | 1.04116E−47 | 1.51293E−45 |
| Rgs10 | 0.482419062 | 9.543994531 | 209.2301427 | 2.02222E−47 | 2.91181E−45 |
| Dgat1 | −0.509721261 | 9.781782774 | 202.6225445 | 5.59181E−46 | 7.97916E−44 |
| Calm2 | −0.288834956 | 10.41747073 | 201.1940212 | 1.14623E−45 | 1.621E−43 |
| Klf4 | −0.805778426 | 9.163911069 | 198.5381953 | 4.35348E−45 | 6.10219E−43 |
| Gstp1 | 0.560007281 | 9.167288045 | 197.5281815 | 7.23193E−45 | 1.00479E−42 |
| Clic1 | 0.288038175 | 10.28204409 | 197.2507071 | 8.31399E−45 | 1.14509E−42 |
| Hmgb2 | 0.354889486 | 10.66675652 | 196.7520978 | 1.06813E−44 | 1.45846E−42 |
| Il7r | 0.473145073 | 9.661302261 | 195.7526991 | 1.76497E−44 | 2.38934E−42 |
| Gm10250 | 0.518735292 | 9.261259604 | 193.764279 | 4.79415E−44 | 6.43513E−42 |
| Cd48 | 0.460544257 | 9.63721787 | 192.5550895 | 8.80297E−44 | 1.17168E−41 |
| Nmrk1 | −0.588456161 | 9.417758277 | 192.2468691 | 1.02779E−43 | 1.35659E−41 |
| Rps18-ps3 | 0.492924084 | 9.426467835 | 191.4030749 | 1.57064E−43 | 2.05598E−41 |
| Gmfg | 0.288142784 | 10.16792471 | 190.062755 | 3.08058E−43 | 3.99946E−41 |
| Cd160 | 0.772172194 | 9.171269402 | 187.9194935 | 9.04635E−43 | 1.16492E−40 |
| Fos | −0.848606018 | 9.403276926 | 187.6453713 | 1.03827E−42 | 1.32623E−40 |
| Calr | −0.332836814 | 10.00618833 | 187.4552473 | 1.14239E−42 | 1.44755E−40 |
| Ltb | 0.435310874 | 10.06047948 | 187.4344179 | 1.15441E−42 | 1.45117E−40 |
| Sptssa | 0.443750066 | 9.506220885 | 187.1311408 | 1.34451E−42 | 1.67683E−40 |
| Rps19 | 0.163786035 | 12.56834608 | 186.4521678 | 1.8914E−42 | 2.34046E−40 |
| Nop56 | −0.471203711 | 9.397440468 | 185.7941949 | 2.63281E−42 | 3.23265E−40 |
| Cox4i1 | 0.202013585 | 10.64833002 | 185.2586938 | 3.44606E−42 | 4.19863E−40 |
| Cnbp | −0.263558911 | 10.22998911 | 181.685277 | 2.07714E−41 | 2.51143E−39 |
| Lck | 0.257552685 | 10.24063704 | 179.7802094 | 5.41265E−41 | 6.49477E−39 |
| AY036118 | 0.563399136 | 9.204622057 | 178.8367065 | 8.69799E−41 | 1.03585E−38 |
| Itgae | 0.682063184 | 9.148814961 | 178.4772671 | 1.04208E−40 | 1.23176E−38 |
| Phlda1 | −0.673473912 | 9.377283171 | 178.2435364 | 1.17203E−40 | 1.37509E−38 |
| Stat3 | −0.443604694 | 9.594221317 | 177.9403458 | 1.36502E−40 | 1.58975E−38 |
| Cd47 | 0.266539976 | 10.2319394 | 176.5081391 | 2.80463E−40 | 3.24252E−38 |
| Rpl18 | 0.144665747 | 11.79456362 | 175.0801355 | 5.75052E−40 | 6.60018E−38 |
| Nr3c1 | −0.516138019 | 9.401251838 | 174.75596 | 6.7686E−40 | 7.7128E−38 |
| Lrrc58 | 0.46964707 | 9.349073684 | 171.1319595 | 4.18736E−39 | 4.7374E−37 |
| Isg15 | −0.670410352 | 9.423880415 | 169.027457 | 1.20665E−38 | 1.35548E−36 |
| Dnaja4 | −0.644926354 | 9.231175487 | 168.0185211 | 2.00427E−38 | 2.23561E−36 |
| Gramd1a | 0.466406544 | 9.358991876 | 166.9212357 | 3.48044E−38 | 3.85502E−36 |
| Satb1 | 0.439489127 | 9.719193943 | 166.7565659 | 3.78098E−38 | 4.15881E−36 |
| Pitpna | 0.435855152 | 9.418669714 | 166.5808975 | 4.13025E−38 | 4.51165E−36 |
| Lmnb1 | −0.393042571 | 9.763928547 | 164.0771622 | 1.45514E−37 | 1.57863E−35 |
| Rab8a | 0.415127367 | 9.432790068 | 164.0612625 | 1.46683E−37 | 1.58048E−35 |
| Rpl36-ps3 | 0.441944981 | 9.423156321 | 163.8405856 | 1.63903E−37 | 1.75409E−35 |
| Dnajb6 | −0.293570643 | 10.00275021 | 162.2193209 | 3.70488E−37 | 3.93836E−35 |
| Zyx | 0.538469116 | 9.22950728 | 162.1601301 | 3.81685E−37 | 4.03034E−35 |
| Gm42418 | 0.493993592 | 9.306589985 | 161.57956 | 5.11145E−37 | 5.3616E−35 |
| Rpl13a | 0.119258065 | 13.13921041 | 161.206428 | 6.16686E−37 | 6.42612E−35 |
| Tpt1 | −0.145383512 | 11.80754119 | 160.7269708 | 7.84903E−37 | 8.12554E−35 |
| Bag3 | −0.659792855 | 9.118691017 | 159.9745004 | 1.14609E−36 | 1.17876E−34 |
| Ccnd2 | −0.396544062 | 9.784153549 | 159.2696002 | 1.63393E−36 | 1.66967E−34 |
| Arhgap9 | 0.438276791 | 9.35894319 | 157.7278258 | 3.54905E−36 | 3.60343E−34 |
| Glipr2 | 0.446096284 | 9.423554685 | 157.1318986 | 4.78992E−36 | 4.83233E−34 |
| Fam110a | −0.472890454 | 9.453583185 | 157.1015845 | 4.86354E−36 | 4.87554E−34 |
| Selplg | 0.274720161 | 10.17793864 | 156.6736327 | 6.03206E−36 | 6.00892E−34 |
| Cycs | −0.32552726 | 9.75607587 | 156.6281029 | 6.17184E−36 | 6.10974E−34 |
| Il4ra | −0.452891333 | 9.452897121 | 156.4370671 | 6.79453E−36 | 6.68439E−34 |
| Emp3 | 0.353071287 | 9.834646436 | 156.2537044 | 7.45123E−36 | 7.28518E−34 |
| Rpl4 | 0.134730304 | 11.68362122 | 155.6700537 | 9.99467E−36 | 9.712E−34 |
| Hnrnpa3 | 0.266912928 | 10.08163683 | 155.1519983 | 1.29711E−35 | 1.25274E−33 |
| Rpsa | 0.151911683 | 11.60418555 | 154.4391519 | 1.85677E−35 | 1.78238E−33 |
| Sec62 | −0.329708774 | 9.701018261 | 152.843486 | 4.14456E−35 | 3.95456E−33 |
| BC021614 | 0.531468977 | 9.186997015 | 152.6941588 | 4.46801E−35 | 4.23766E−33 |
| Hnrnpf | 0.226485212 | 10.3476999 | 152.4248087 | 5.1166E−35 | 4.82392E−33 |
| Btf3 | 0.174817152 | 10.69728489 | 150.1944197 | 1.57204E−34 | 1.47335E−32 |
| Tuba4a | −0.500013194 | 9.3182479 | 149.6257279 | 2.09299E−34 | 1.95005E−32 |
| Rplp2 | −0.144805547 | 11.92184821 | 149.256893 | 2.51993E−34 | 2.3341E−32 |
| Gch1 | −0.466693357 | 9.423417801 | 148.6833891 | 3.36316E−34 | 3.09704E−32 |
| Arpc1b | 0.211873522 | 10.49392739 | 148.4554378 | 3.77203E−34 | 3.45348E−32 |
| Stk17b | 0.281362595 | 10.00322266 | 148.0916995 | 4.52987E−34 | 4.12349E−32 |
| Ppp1ca | 0.240424662 | 10.26494776 | 148.060095 | 4.60251E−34 | 4.16567E−32 |
| Rpl7a-ps5 | 0.486459932 | 9.2808747 | 148.0086319 | 4.72329E−34 | 4.25069E−32 |
| Cyp51 | −0.517971726 | 9.223050414 | 146.0605091 | 1.25925E−33 | 1.12685E−31 |
| Sub1 | −0.214884457 | 11.18398687 | 145.3238318 | 1.82458E−33 | 1.62357E−31 |
| Actr3 | 0.249028441 | 10.16798322 | 145.0491999 | 2.0951E−33 | 1.85387E−31 |
| Ms4a6b | 0.294576449 | 10.12969439 | 144.501494 | 2.76025E−33 | 2.42886E−31 |
| Tubb4b | −0.459637938 | 9.394536981 | 144.3920797 | 2.91655E−33 | 2.55222E−31 |
| Eif3h | 0.208541804 | 10.26895897 | 144.1508682 | 3.29311E−33 | 2.86591E−31 |
| Coq10b | −0.411794907 | 9.435490542 | 144.0065092 | 3.54134E−33 | 3.0651E−31 |
| Atp5c1 | 0.269041545 | 9.839431013 | 142.7411254 | 6.69627E−33 | 5.76425E−31 |
| Rps18 | 0.122847279 | 12.46503118 | 139.9990592 | 2.6633E−32 | 2.28021E−30 |
| Nr4a2 | −0.513897887 | 9.457054691 | 139.5805932 | 3.28798E−32 | 2.79991E−30 |
| Calm1 | −0.187542883 | 10.85460141 | 139.4756112 | 3.46647E−32 | 2.93612E−30 |
| Rpl13 | 0.114773349 | 12.62813742 | 138.4213697 | 5.89437E−32 | 4.966E−30 |
| Ict1 | 0.423189272 | 9.30361635 | 138.3204015 | 6.20181E−32 | 5.19738E−30 |
| Hmgn2 | 0.550119942 | 9.341504132 | 138.1803419 | 6.65501E−32 | 5.54783E−30 |
| Lef1 | 0.504911527 | 9.197619018 | 137.0327804 | 1.18611E−31 | 9.836E−30 |
| Grk6 | 0.422500161 | 9.358866658 | 136.6318883 | 1.45146E−31 | 1.19738E−29 |
| Gm10263 | 0.500153042 | 9.186742654 | 136.3215546 | 1.69699E−31 | 1.39267E−29 |
| Gem | −0.660716773 | 9.293071626 | 134.6824614 | 3.8743E−31 | 3.16314E−29 |
| Klhl6 | 0.483227269 | 9.233459769 | 133.712411 | 6.31515E−31 | 5.12952E−29 |
| Cd27 | −0.611171828 | 9.270411364 | 133.5494989 | 6.85521E−31 | 5.53978E−29 |
| Rpl10a | 0.159600898 | 11.11793653 | 133.4138158 | 7.34009E−31 | 5.90151E−29 |
| Xbp1 | −0.375600558 | 9.506719641 | 132.8318821 | 9.84016E−31 | 7.87163E−29 |
| Actb | 0.349014866 | 13.2991423 | 132.5191377 | 1.15191E−30 | 9.16838E−29 |
| Shisa5 | 0.187947346 | 10.97087 | 132.4670563 | 1.18253E−30 | 9.36503E−29 |
| Dnajc15 | 0.287104543 | 9.848245672 | 132.2517602 | 1.31799E−30 | 1.03859E−28 |
| Fmnl1 | 0.429752574 | 9.260548258 | 129.9782686 | 4.14289E−30 | 3.24847E−28 |
| Cdkn2d | 0.464791922 | 9.29200856 | 129.2330519 | 6.03052E−30 | 4.70529E−28 |
| Fxyd5 | 0.182264591 | 10.89173272 | 128.760728 | 7.65071E−30 | 5.94018E−28 |
| Arpc5 | 0.249915994 | 10.04618205 | 128.6880991 | 7.93585E−30 | 6.13151E−28 |
| Tob1 | −0.446983839 | 9.361751749 | 128.221138 | 1.00408E−29 | 7.72024E−28 |
| Sp100 | 0.378951326 | 9.450532416 | 127.737472 | 1.28117E−29 | 9.80309E−28 |
| Cox7a2l | 0.233483798 | 10.08201636 | 127.107975 | 1.75936E−29 | 1.33974E−27 |
| Xist | −0.56767985 | 9.970373424 | 126.6271392 | 2.2417E−29 | 1.69887E−27 |
| Hist1h1c | 0.557306764 | 9.489015558 | 126.3156087 | 2.62273E−29 | 1.97816E−27 |
| Pgk1 | 0.418281433 | 9.112923746 | 125.3115168 | 4.35006E−29 | 3.26543E−27 |
| Actg1 | 0.182969375 | 12.14233273 | 125.146896 | 4.72632E−29 | 3.53114E−27 |
| Mtch1 | 0.458946966 | 9.20177114 | 124.5864031 | 6.26892E−29 | 4.66166E−27 |
| Atp2b1 | −0.38615604 | 9.501506429 | 124.1820724 | 7.68576E−29 | 5.68854E−27 |
| Ncl | −0.245541061 | 10.05061385 | 123.9476575 | 8.64954E−29 | 6.3721E−27 |
| S1pr1 | 0.528441748 | 9.211582313 | 123.7386899 | 9.61016E−29 | 7.04701E−27 |
| Slc25a4 | 0.348268536 | 9.539790652 | 123.6240204 | 1.01819E−28 | 7.43185E−27 |
| Tmem234 | 0.309086225 | 9.603992608 | 123.5902044 | 1.03569E−28 | 7.52492E−27 |
| Ahsa1 | −0.373673862 | 9.394169648 | 123.4119192 | 1.13306E−28 | 8.19477E−27 |
| Oat | −0.435562041 | 9.261683421 | 123.1811798 | 1.27279E−28 | 9.16353E−27 |
| Eif4a1 | −0.209870948 | 10.40687573 | 123.0223878 | 1.37884E−28 | 9.88212E−27 |
| Srsf5 | −0.243227861 | 10.03951019 | 121.6337503 | 2.77639E−28 | 1.98086E−26 |
| Rasal3 | 0.417194458 | 9.277924457 | 121.128153 | 3.58228E−28 | 2.54438E−26 |
| Txn1 | −0.257078553 | 9.989683557 | 120.9745987 | 3.87056E−28 | 2.73687E−26 |
| Dusp5 | −0.290377017 | 9.972912538 | 120.6642471 | 4.526E−28 | 3.18611E−26 |
| Mrfap1 | −0.307122783 | 9.592747399 | 120.576342 | 4.73107E−28 | 3.31572E−26 |
| Srsf2 | −0.297033097 | 9.8103015 | 120.5673514 | 4.75256E−28 | 3.31611E−26 |
| Sfn | −0.630910469 | 9.178735424 | 120.3008256 | 5.43595E−28 | 3.77632E−26 |
| Rps12-ps3 | 0.308504409 | 9.879369687 | 120.1649948 | 5.82119E−28 | 4.02373E−26 |
| Ccdc85b | 0.376786261 | 9.099879984 | 120.1576082 | 5.84291E−28 | 4.02373E−26 |
| Eif3e | 0.216240052 | 10.08436298 | 119.78202 | 7.06084E−28 | 4.84141E−26 |
| Smox | −0.474515311 | 9.123010044 | 119.5466722 | 7.95026E−28 | 5.42777E−26 |
| Prelid1 | 0.300888669 | 9.653107806 | 119.1448432 | 9.73547E−28 | 6.61803E−26 |
| Socs1 | −0.469790829 | 9.336838521 | 117.5893552 | 2.13272E−27 | 1.4436E−25 |
| Gpi1 | 0.268069428 | 9.724725928 | 117.3315071 | 2.42881E−27 | 1.63702E−25 |
| Gm8186 | 0.400557572 | 9.271352225 | 117.0049248 | 2.86356E−27 | 1.92186E−25 |
| Gm26917 | 0.355608704 | 9.439877032 | 116.7023452 | 3.33552E−27 | 2.22917E−25 |
| Kdm6b | −0.474669899 | 9.658683083 | 116.14973 | 4.40733E−27 | 2.9331E−25 |
| Morf4l2 | −0.385016023 | 9.334072103 | 115.2078964 | 7.08649E−27 | 4.69635E−25 |
| mt-Atp6 | 0.13109516 | 12.41359392 | 114.2993627 | 1.12049E−26 | 7.39475E−25 |
| Rps21 | −0.181566483 | 10.91649475 | 114.161468 | 1.20118E−26 | 7.8944E−25 |
| Traf1 | −0.259615851 | 10.09188115 | 113.4484171 | 1.72101E−26 | 1.12641E−24 |
| Hmgb1 | 0.245489958 | 10.23428059 | 113.378642 | 1.78265E−26 | 1.16195E−24 |
| Rps27 | −0.126585153 | 13.17754164 | 113.3397676 | 1.81794E−26 | 1.1801E−24 |
| Gimap4 | 0.215414428 | 10.44074796 | 113.191718 | 1.95888E−26 | 1.2664E−24 |
| Gnb2 | 0.258833488 | 9.763400839 | 112.9354823 | 2.22912E−26 | 1.43524E−24 |
| Itgb7 | 0.274275587 | 9.895610202 | 112.219657 | 3.19837E−26 | 2.05097E−24 |
| Kcnab2 | 0.416180538 | 9.204098465 | 110.340368 | 8.25308E−26 | 5.27099E−24 |
| Rps6 | 0.103817085 | 12.19422287 | 109.8318232 | 1.06667E−25 | 6.78514E−24 |
| Itpkb | −0.372082819 | 9.445651895 | 108.7449466 | 1.84571E−25 | 1.16937E−23 |
| Kxd1 | 0.377030142 | 9.31810604 | 108.4195949 | 2.17497E−25 | 1.37248E−23 |
| Gm5424 | 0.355390948 | 9.09348985 | 108.2876044 | 2.32474E−25 | 1.46117E−23 |
| Ctsw | 0.281167862 | 9.869959927 | 106.8287454 | 4.85349E−25 | 3.03852E−23 |
| Qrfp | 0.243034806 | 9.076767353 | 106.3828523 | 6.07814E−25 | 3.79022E−23 |
| Capzb | 0.213939408 | 10.11156981 | 106.153532 | 6.82381E−25 | 4.23853E−23 |
| Tpgs1 | 0.420110336 | 9.169815546 | 105.7409159 | 8.40342E−25 | 5.19929E−23 |
| Itga4 | 0.412518622 | 9.298709558 | 105.4396292 | 9.78336E−25 | 6.02952E−23 |
| Ankrd11 | 0.350142771 | 9.482587467 | 104.8812954 | 1.29676E−24 | 7.96102E−23 |
| Pkp3 | 0.331642575 | 9.632608745 | 104.763521 | 1.37618E−24 | 8.41594E−23 |
| Znrf1 | 0.393356078 | 9.30009727 | 104.5615058 | 1.52389E−24 | 9.28341E−23 |
| Gpsm3 | 0.273081602 | 9.652489804 | 103.3850532 | 2.75949E−24 | 1.67462E−22 |
| Csrp1 | −0.407937762 | 9.37033353 | 103.2949394 | 2.8879E−24 | 1.74586E−22 |
| Ier5l | −0.547686053 | 9.138751627 | 102.9398289 | 3.45484E−24 | 2.08065E−22 |
| Vasp | 0.227570734 | 9.975852973 | 102.8106476 | 3.68762E−24 | 2.21243E−22 |
| Rbm39 | −0.167004852 | 10.64268875 | 102.3487305 | 4.65597E−24 | 2.78286E−22 |
| Pnrc1 | −0.206120221 | 10.52842542 | 101.7477551 | 6.30616E−24 | 3.75501E−22 |
| Arpc3 | −0.186319393 | 10.54396257 | 101.3233979 | 7.81274E−24 | 4.63468E−22 |
| Lrch4 | 0.386196626 | 9.117920834 | 101.0593439 | 8.92685E−24 | 5.27583E−22 |
| Tnfsf11 | −0.479155286 | 9.528185643 | 100.5769031 | 1.13888E−23 | 6.70585E−22 |
| Ms4a4b | 0.224364573 | 10.74767447 | 100.0145775 | 1.51279E−23 | 8.8745E−22 |
| Wsb1 | −0.383939877 | 9.299259433 | 99.70808238 | 1.76599E−23 | 1.03216E−21 |
| Cox17 | −0.255036164 | 9.759086926 | 99.34607039 | 2.12018E−23 | 1.23038E−21 |
| Rps26-ps1 | 0.376422445 | 9.302607157 | 99.34560267 | 2.12068E−23 | 1.23038E−21 |
| Aprt | 0.302163807 | 9.570312455 | 97.89843718 | 4.40401E−23 | 2.54581E−21 |
| Commd8 | 0.340663528 | 9.362522227 | 97.69437133 | 4.88208E−23 | 2.8119E−21 |
| Cmtm7 | 0.347833176 | 9.445414037 | 97.66881931 | 4.94549E−23 | 2.8381E−21 |
| Cdk2ap1 | 0.40313369 | 9.207010836 | 97.41784043 | 5.61379E−23 | 3.21E−21 |
| Nhp2l1 | −0.25771889 | 9.660380552 | 97.18476802 | 6.31506E−23 | 3.59799E−21 |
| Arhgap15 | 0.345069342 | 9.353711095 | 97.13997149 | 6.45956E−23 | 3.66713E−21 |
| Ikzf1 | 0.364959967 | 9.314515256 | 97.12258211 | 6.51654E−23 | 3.68627E−21 |
| Mrps6 | −0.445518043 | 9.167109148 | 97.06634456 | 6.70428E−23 | 3.77897E−21 |
| Pdlim2 | 0.398459075 | 9.182597688 | 96.96123973 | 7.06979E−23 | 3.97086E−21 |
| Hspa2 | −0.459505667 | 9.124122695 | 96.94098529 | 7.14248E−23 | 3.99752E−21 |
| Vim | 0.262752919 | 10.51559022 | 96.26760304 | 1.00359E−22 | 5.59713E−21 |
| Traf4 | −0.411142056 | 9.288678658 | 96.17375062 | 1.05231E−22 | 5.84825E−21 |
| Coro1b | 0.359271007 | 9.291592437 | 95.80235442 | 1.26945E−22 | 7.03034E−21 |
| Litaf | −0.434433383 | 9.369696159 | 95.74269972 | 1.30828E−22 | 7.22016E−21 |
| Ier5 | −0.332369309 | 9.648355931 | 95.20641105 | 1.71534E−22 | 9.43377E−21 |
| Cxcr6 | 0.285170249 | 10.38589926 | 94.75608108 | 2.15351E−22 | 1.18026E−20 |
| Gyg | 0.404072997 | 9.169055117 | 94.60060052 | 2.32948E−22 | 1.2723E−20 |
| Gngt2 | −0.414175481 | 9.266095139 | 94.22857227 | 2.81113E−22 | 1.53009E−20 |
| Srgn | −0.156866717 | 11.40960169 | 94.22033317 | 2.82286E−22 | 1.53121E−20 |
| Traf3ip3 | 0.398126336 | 9.211377897 | 94.14101487 | 2.93827E−22 | 1.58837E−20 |
| Slc25a3 | −0.223869744 | 9.929245406 | 93.89114737 | 3.33362E−22 | 1.79596E−20 |
| C1d | 0.358002392 | 9.257445341 | 93.60629381 | 3.84962E−22 | 2.06692E−20 |
| Taf10 | 0.329789291 | 9.42027031 | 93.17625006 | 4.78387E−22 | 2.55986E−20 |
| Rrad | −0.422165808 | 9.481942925 | 92.71155163 | 6.04994E−22 | 3.22643E−20 |
| Myo1f | 0.386024191 | 9.211461927 | 90.89289789 | 1.51663E−21 | 8.06104E−20 |
| Hsd11b1 | 0.410088948 | 9.207302928 | 90.62179281 | 1.73934E−21 | 9.21385E−20 |
| Ctla2a | −0.340237861 | 10.39420628 | 90.54680962 | 1.80652E−21 | 9.53783E−20 |
| Tubb5 | 0.33277636 | 9.951843603 | 90.14093695 | 2.21785E−21 | 1.16706E−19 |
| Gramd3 | 0.284054197 | 10.20990355 | 90.01056232 | 2.36892E−21 | 1.24243E−19 |
| Jun | −0.655178909 | 9.472386028 | 89.47089847 | 3.11182E−21 | 1.62667E−19 |
| Fam169b | 0.401940452 | 9.20492433 | 89.45833686 | 3.13165E−21 | 1.63165E−19 |
| Fermt3 | 0.346761961 | 9.277314729 | 89.42967881 | 3.17734E−21 | 1.65003E−19 |
| Ikzf3 | −0.43554387 | 9.229056647 | 89.22703786 | 3.52005E−21 | 1.82203E−19 |
| Caprin1 | 0.362718036 | 9.222741789 | 89.18692735 | 3.59215E−21 | 1.85329E−19 |
| Bola2 | −0.279074797 | 9.545437178 | 89.07835747 | 3.7948E−21 | 1.95149E−19 |
| 1700097N02Rik | 0.426479844 | 9.138943007 | 88.54524555 | 4.96853E−21 | 2.54682E−19 |
| Akt1 | 0.388322632 | 9.186110081 | 88.23288633 | 5.81845E−21 | 2.97285E−19 |
| Set | 0.275235713 | 9.68026491 | 87.87236611 | 6.98173E−21 | 3.55574E−19 |
| Lcn4 | 0.386003461 | 9.104970691 | 87.03518994 | 1.06607E−20 | 5.41203E−19 |
| Ptpn6 | 0.374733064 | 9.254685928 | 86.45613527 | 1.42871E−20 | 7.20681E−19 |
| Tmem71 | 0.392168946 | 9.18300717 | 86.45911911 | 1.42656E−20 | 7.20681E−19 |
| Prkcq | 0.385697348 | 9.187898023 | 86.1059948 | 1.70545E−20 | 8.57544E−19 |
| Rps3a1 | 0.101927456 | 11.66239034 | 86.05600387 | 1.74911E−20 | 8.76715E−19 |
| Hilpda | −0.363494493 | 9.621933344 | 85.85753408 | 1.93377E−20 | 9.66215E−19 |
| Rpl13a-ps1 | 0.369121872 | 9.125886702 | 85.80554006 | 1.98529E−20 | 9.88838E−19 |
| Cotl1 | 0.189250759 | 10.42452425 | 85.51065232 | 2.30457E−20 | 1.14426E−18 |
| Kpna1 | −0.370106801 | 9.300870038 | 85.23248687 | 2.65268E−20 | 1.31299E−18 |
| Gabpb1 | −0.35309655 | 9.443575937 | 85.20182613 | 2.69413E−20 | 1.32936E−18 |
| Fosl2 | −0.360285481 | 9.496287214 | 85.06065057 | 2.89352E−20 | 1.42331E−18 |
| mt-Nd2 | −0.223466869 | 9.877039562 | 84.76700348 | 3.3568E−20 | 1.64608E−18 |
| Ctbp1 | 0.291313018 | 9.41304975 | 84.69950904 | 3.47337E−20 | 1.69798E−18 |
| Stat4 | −0.316811236 | 9.451262982 | 84.38770905 | 4.06668E−20 | 1.98191E−18 |
| Lgals1 | 0.205366044 | 11.71367544 | 84.2394234 | 4.38342E−20 | 2.12972E−18 |
| Def6 | 0.357773345 | 9.254739348 | 84.07852182 | 4.75509E−20 | 2.30324E−18 |
| Plscr1 | −0.37206631 | 9.476236025 | 83.60981525 | 6.02731E−20 | 2.91056E−18 |
| Gltscr2 | 0.174676511 | 10.26087104 | 83.346365 | 6.88653E−20 | 3.31537E−18 |
| Pik3ip1 | 0.330447087 | 9.095932723 | 83.00333665 | 8.1915E−20 | 3.93167E−18 |
| Naca | 0.132857132 | 10.78159304 | 82.76181285 | 9.2561E−20 | 4.41914E−18 |
| Lbr | 0.318806475 | 9.348045598 | 82.76035438 | 9.26293E−20 | 4.41914E−18 |
| Ly6e | −0.164934597 | 10.75197012 | 82.47918371 | 1.06789E−19 | 5.07937E−18 |
| Ubxn4 | −0.283952138 | 9.497877918 | 82.09781857 | 1.29515E−19 | 6.1419E−18 |
| Maff | −0.449911666 | 9.211492069 | 81.93490116 | 1.40644E−19 | 6.64972E−18 |
| Slc5a3 | −0.383528621 | 9.112932121 | 81.55251603 | 1.70666E−19 | 8.04516E−18 |
| Ywhah | 0.252414688 | 9.657038205 | 81.37024179 | 1.87155E−19 | 8.79628E−18 |
| Susd3 | 0.35903436 | 9.274618606 | 81.24943703 | 1.98952E−19 | 9.29559E−18 |
| Snhg12 | −0.372153059 | 9.217070705 | 81.25063679 | 1.98832E−19 | 9.29559E−18 |
| Pim3 | −0.419460628 | 9.239693094 | 81.10803033 | 2.1371E−19 | 9.95573E−18 |
| Sema4d | 0.336007509 | 9.272910863 | 81.03992068 | 2.21204E−19 | 1.02746E−17 |
| Nrip1 | −0.351147226 | 9.518465731 | 80.79922139 | 2.49856E−19 | 1.15716E−17 |
| Errfi1 | −0.415709417 | 9.203060475 | 80.77279894 | 2.5322E−19 | 1.16931E−17 |
| Adora2a | −0.337733868 | 9.371066602 | 80.11544679 | 3.53161E−19 | 1.62608E−17 |
| Rps15a | −0.091705836 | 12.40648985 | 80.05818453 | 3.63545E−19 | 1.66904E−17 |
| Tcf7 | 0.361212693 | 9.327301692 | 79.84620757 | 4.04716E−19 | 1.85269E−17 |
| Klrb1f | 0.310571128 | 9.09219065 | 79.5343695 | 4.73909E−19 | 2.16318E−17 |
| Rpl23a | −0.093737153 | 12.18419476 | 79.4984072 | 4.82614E−19 | 2.19659E−17 |
| Atp5l | 0.148555301 | 10.43217254 | 79.40175043 | 5.06812E−19 | 2.30011E−17 |
| Rps27rt | 0.194128581 | 11.15059622 | 79.12000934 | 5.84493E−19 | 2.64508E−17 |
| Hist1h2ap | 0.371210153 | 9.098222032 | 79.03133498 | 6.11326E−19 | 2.75863E−17 |
| Dad1 | −0.188540821 | 9.987070117 | 78.99222215 | 6.23549E−19 | 2.8058E−17 |
| Pdia6 | −0.305181564 | 9.433423543 | 78.96680159 | 6.31625E−19 | 2.83408E−17 |
| Dedd2 | −0.361161452 | 9.208472136 | 78.60470661 | 7.58684E−19 | 3.39458E−17 |
| Capza1 | 0.275051341 | 9.421084656 | 78.45568471 | 8.18131E−19 | 3.65025E−17 |
| Whsc1l1 | 0.286256556 | 9.465405344 | 77.97147828 | 1.04539E−18 | 4.65111E−17 |
| Dusp14 | −0.490461557 | 9.164191183 | 77.49990377 | 1.32729E−18 | 5.88878E−17 |
| Pabpc1 | 0.128653685 | 10.80961358 | 77.10286727 | 1.62282E−18 | 7.17983E−17 |
| Flt3l | 0.342999594 | 9.248983424 | 76.47498946 | 2.2302E−18 | 9.83958E−17 |
| Arrb2 | 0.323205878 | 9.299307513 | 76.15477607 | 2.6228E−18 | 1.15396E−16 |
| mt-Co3 | 0.113435634 | 12.04559976 | 76.05277042 | 2.76185E−18 | 1.21177E−16 |
| Sept9 | 0.288906266 | 9.423194345 | 76.02239789 | 2.80465E−18 | 1.22715E−16 |
| 2810001G20Rik | 0.388632897 | 9.147759194 | 75.85013039 | 3.06032E−18 | 1.33533E−16 |
| 1600014C10Rik | 0.376989592 | 9.154287123 | 75.75980593 | 3.20356E−18 | 1.39399E−16 |
| Tmem30a | 0.308038318 | 9.310066934 | 75.25148409 | 4.14419E−18 | 1.79835E−16 |
| Nfkbie | −0.336107134 | 9.343303521 | 75.20801706 | 4.23643E−18 | 1.83336E−16 |
| Gpx4 | −0.205314323 | 10.06582643 | 75.20257186 | 4.24813E−18 | 1.83341E−16 |
| Gm42726 | 0.320348649 | 9.104150123 | 75.1172932 | 4.43564E−18 | 1.90913E−16 |
| Ramp3 | −0.440634397 | 9.976673631 | 75.0436194 | 4.60429E−18 | 1.97635E−16 |
| Ifitm3 | 0.451646848 | 9.710327146 | 74.9885843 | 4.73444E−18 | 2.02672E−16 |
| Cisd3 | 0.352850049 | 9.208350817 | 74.66564795 | 5.57579E−18 | 2.38046E−16 |
| Top1 | −0.28395075 | 9.452133131 | 74.61436731 | 5.72252E−18 | 2.43653E−16 |
| Sppl3 | 0.331427267 | 9.125075068 | 73.91656836 | 8.14895E−18 | 3.46035E−16 |
| Usmg5 | −0.19994212 | 10.00954642 | 73.69959983 | 9.09573E−18 | 3.85207E−16 |
| Bst2 | −0.31574848 | 9.349556332 | 73.67662979 | 9.2022E−18 | 3.88676E−16 |
| Gm26740 | −0.290618009 | 9.614149283 | 73.6072547 | 9.53137E−18 | 4.01509E−16 |

TABLE 2C
Small Intestine LPL Ctrl vs KO

| GeneSymbol | logFC | logCPM | LR | PValue | FDR |
|---|---|---|---|---|---|
| Malat1 | −1.270390299 | 14.65408192 | 1391.007551 | 1.8902E−304 | 2.5698E−300 |
| Dnajb1 | −2.989003621 | 10.24133724 | 1248.384643 | 1.8627E−273 | 1.2662E−269 |
| Ubb | −0.928036045 | 12.68983308 | 1129.213594 | 1.4776E−247 | 6.6959E−244 |
| Hsp90aa1 | −1.73334477 | 10.38503603 | 848.7603464 | 1.3523E−186 | 4.5961E−183 |
| Hsp90ab1 | −1.124878483 | 10.88661857 | 744.3402084 | 6.8242E−164 | 1.8555E−160 |
| Hspa1a | −3.477661443 | 10.7136333 | 610.5048084 | 8.6886E−135 | 1.9687E−131 |
| Hspe1 | −1.481859098 | 10.17562846 | 609.4431434 | 1.4787E−134 | 2.8718E−131 |
| Hspa1b | −2.45826073 | 9.673476745 | 541.557568 | 8.6415E−120 | 1.4685E−116 |
| Hspa8 | −0.733021848 | 11.94760636 | 536.6824715 | 9.9348E−119 | 1.5007E−115 |
| Dnaja1 | −1.142985442 | 10.58020182 | 500.7270965 | 6.6034E−111 | 8.9774E−108 |
| Hsph1 | −1.674119669 | 9.693521287 | 391.0098358 | 4.98925E−87 | 6.16626E−84 |
| Pfn1 | 0.556969656 | 12.36179331 | 354.5323953 | 4.36681E−79 | 4.94723E−76 |
| Ifi27l2a | −1.526203755 | 10.06603444 | 308.5419345 | 4.53792E−69 | 4.74562E−66 |
| Ddx5 | −0.569218691 | 11.28301838 | 295.0912662 | 3.86588E−66 | 3.75404E−63 |
| Crip1 | 1.063247863 | 11.26439747 | 275.0510604 | 8.99683E−62 | 8.15413E−59 |
| Ubc | −0.684801113 | 11.25718429 | 269.2572026 | 1.6474E−60 | 1.39978E−57 |
| Jun | −1.621216633 | 10.08346217 | 267.8801831 | 3.28792E−60 | 2.62937E−57 |
| Lgals1 | 1.123792206 | 11.27737133 | 266.4474621 | 6.7482E−60 | 5.09677E−57 |
| Cd52 | 0.579508154 | 11.896613 | 259.9242984 | 1.78245E−58 | 1.27539E−55 |
| Hspd1 | −1.123244696 | 9.873827325 | 248.7759061 | 4.80071E−56 | 3.26328E−53 |
| Cd160 | 1.612792846 | 9.910004584 | 244.9841777 | 3.2209E−55 | 2.08515E−52 |
| Rbm3 | 0.606449645 | 10.9936177 | 229.5941443 | 7.30892E−52 | 4.51658E−49 |
| S100a6 | 1.204174767 | 10.87443069 | 219.9356841 | 9.34141E−50 | 5.52159E−47 |
| Gzma | 1.993209068 | 11.53461012 | 215.7661412 | 7.58466E−49 | 4.29639E−46 |
| Rps29 | −0.33975966 | 12.45913805 | 214.1327455 | 1.72289E−48 | 9.3691E−46 |
| Slc25a5 | 0.672711688 | 10.63606639 | 207.9571703 | 3.83322E−47 | 2.00433E−44 |
| Cd7 | 1.532985299 | 10.09719296 | 198.2226571 | 5.10148E−45 | 2.56869E−42 |
| Fos | −1.555883912 | 9.951733106 | 191.3239371 | 1.63437E−43 | 7.93543E−41 |
| Junb | −0.603167841 | 12.24822312 | 187.5138271 | 1.10924E−42 | 5.20006E−40 |
| Vim | 0.928746254 | 10.32884884 | 180.8919276 | 3.09513E−41 | 1.40261E−38 |
| S100a4 | 1.526515304 | 9.992761306 | 176.514377 | 2.79585E−40 | 1.22611E−37 |
| Klf6 | −0.926855403 | 10.30217968 | 170.4261312 | 5.97167E−39 | 2.53703E−36 |
| AY036118 | −1.061137817 | 9.604280179 | 168.5762641 | 1.51402E−38 | 6.23731E−36 |
| Eif4a2 | −0.786277362 | 10.01209351 | 167.6815977 | 2.37437E−38 | 9.49399E−36 |
| Rplp0 | 0.305778795 | 12.4630419 | 163.5750167 | 1.87328E−37 | 7.27635E−35 |
| Zfp36l2 | 0.955324495 | 10.26398104 | 162.744194 | 2.84515E−37 | 1.07444E−34 |
| Gm42418 | −1.048481014 | 9.719045857 | 160.8447438 | 7.3975E−37 | 2.71808E−34 |
| Eif3f | 0.463631301 | 10.85999602 | 157.5770827 | 3.8287E−36 | 1.36977E−33 |
| Gadd45b | −1.017411734 | 9.987572676 | 156.6695014 | 6.04462E−36 | 2.10709E−33 |
| Dusp1 | −0.925508201 | 10.28518602 | 154.6952798 | 1.63224E−35 | 5.54759E−33 |
| Ier5 | −0.849926882 | 9.977632158 | 153.3008753 | 3.29244E−35 | 1.09172E−32 |
| Rpl24 | 0.336154281 | 11.8173782 | 153.1549619 | 3.54329E−35 | 1.14693E−32 |
| Itgb7 | 0.730684655 | 10.21660441 | 145.9816063 | 1.31027E−33 | 4.1426E−31 |
| Ppp1r15a | −0.850080959 | 9.962305144 | 145.3477627 | 1.80274E−33 | 5.57004E−31 |
| mt-Nd2 | −0.666229752 | 10.16694083 | 142.9422572 | 6.0514E−33 | 1.8282E−30 |
| Rps27 | −0.280332283 | 13.43297512 | 140.2437348 | 2.35459E−32 | 6.95883E−30 |
| Emp3 | 0.853721822 | 10.01923353 | 138.8508125 | 4.74812E−32 | 1.37342E−29 |
| Jund | −0.511687126 | 11.88052936 | 138.1873795 | 6.63147E−32 | 1.87823E−29 |
| Arpc5 | 0.641891287 | 10.33312177 | 137.1516697 | 1.11718E−31 | 3.0996E−29 |
| Prkca | −0.936438709 | 9.803809383 | 135.1375687 | 3.08067E−31 | 8.37635E−29 |
| Tagln2 | 0.728752002 | 10.27956689 | 132.3672087 | 1.24352E−30 | 3.31485E−28 |
| Cacybp | −0.820907994 | 9.761275879 | 127.6377263 | 1.3472E−29 | 3.52215E−27 |
| Itgae | 1.141170294 | 9.754012995 | 127.2951357 | 1.60103E−29 | 4.10679E−27 |
| Cd47 | 0.621464194 | 10.28476905 | 123.8215437 | 9.21714E−29 | 2.3205E−26 |
| Rpl23a-ps3 | −0.389746504 | 10.83309006 | 119.834953 | 6.87492E−28 | 1.69935E−25 |
| H2afz | 0.681004033 | 10.6429052 | 118.7869428 | 1.16605E−27 | 2.83079E−25 |
| S100a10 | 0.525127377 | 10.79175384 | 117.9491163 | 1.77894E−27 | 4.24292E−25 |
| Fosb | −1.063020116 | 9.629543946 | 117.8377317 | 1.88169E−27 | 4.41063E−25 |
| Cox5a | 0.592668928 | 10.24515525 | 115.5012812 | 6.11196E−27 | 1.40234E−24 |
| Rpl38 | −0.268147527 | 11.78716014 | 115.4764231 | 6.18905E−27 | 1.40234E−24 |
| Ptprcap | 0.548197307 | 10.41240534 | 109.6451952 | 1.17198E−25 | 2.61197E−23 |
| Arhgdib | 0.404256062 | 10.95537113 | 109.2336833 | 1.44238E−25 | 3.16277E−23 |
| Rpl37a | −0.219867372 | 12.67658574 | 108.9253496 | 1.68514E−25 | 3.63643E−23 |
| Rps27rt | −0.377704095 | 11.22532454 | 107.7024833 | 3.12311E−25 | 6.63417E−23 |
| Atf3 | −0.833608127 | 9.564432286 | 107.484863 | 3.48557E−25 | 7.29021E−23 |
| Rpl32 | 0.234928384 | 12.53119065 | 107.2016913 | 4.02093E−25 | 8.28252E−23 |
| Rpl29 | 0.348661409 | 11.2510885 | 106.283271 | 6.39137E−25 | 1.29688E−22 |
| Zfp36 | −1.069360095 | 9.720937454 | 105.7272946 | 8.46138E−25 | 1.69165E−22 |
| Nfkbia | −0.548369836 | 11.02410921 | 105.552441 | 9.24194E−25 | 1.82093E−22 |
| Tnfaip3 | −0.556203662 | 10.4976442 | 105.1592788 | 1.12702E−24 | 2.18884E−22 |
| Rpl3 | 0.23946729 | 12.12971157 | 103.4841404 | 2.62487E−24 | 5.02607E−22 |
| Lcp1 | 0.476475494 | 10.58352627 | 101.648977 | 6.62859E−24 | 1.25161E−21 |
| Dnaja4 | −0.883775551 | 9.627504548 | 101.2617633 | 8.05967E−24 | 1.50097E−21 |
| Fus | −0.583225585 | 10.0621585 | 100.5551387 | 1.15146E−23 | 2.11543E−21 |
| Eef2 | 0.30089659 | 11.36578563 | 98.60722272 | 3.07894E−23 | 5.5811E−21 |
| Gm10073 | −0.587468651 | 9.971221351 | 98.04010631 | 4.09994E−23 | 7.33404E−21 |
| H2-Q6 | −0.666621999 | 9.913841776 | 96.58296852 | 8.55813E−23 | 1.51101E−20 |
| Lsp1 | 0.478483224 | 10.575597 | 96.3577739 | 9.58906E−23 | 1.67132E−20 |
| Cxcr6 | 0.682928486 | 10.4358259 | 95.40449085 | 1.55201E−22 | 2.67084E−20 |
| Rplp1 | −0.248800579 | 11.77289572 | 95.07571795 | 1.83241E−22 | 3.0755E−20 |
| Itpkb | −0.755356088 | 9.788344057 | 95.07750247 | 1.83076E−22 | 3.0755E−20 |
| Bag3 | −0.73666804 | 9.556132624 | 92.98437325 | 5.27089E−22 | 8.73875E−20 |
| H2-Q4 | −0.600298455 | 10.03136803 | 92.46838803 | 6.84088E−22 | 1.1205E−19 |
| Cd69 | −0.675277132 | 10.22899337 | 91.3465498 | 1.2059E−21 | 1.95169E−19 |
| Son | −0.580002512 | 9.985249388 | 89.75818 | 2.69123E−21 | 4.30439E−19 |
| Gm8797 | −0.760754138 | 9.695703486 | 89.16169111 | 3.63827E−21 | 5.75142E−19 |
| Reep5 | 0.715048932 | 9.86923952 | 89.08940618 | 3.77366E−21 | 5.89689E−19 |
| Ermn | 0.936240345 | 9.607995489 | 88.5867298 | 4.86542E−21 | 7.51652E−19 |
| Cyba | 0.440199696 | 10.55096537 | 86.79334234 | 1.20474E−20 | 1.84028E−18 |
| Emp1 | −0.831580324 | 9.618680989 | 86.48342872 | 1.40913E−20 | 2.12857E−18 |
| Hnrnpf | 0.389350328 | 10.63476331 | 86.40296267 | 1.46765E−20 | 2.1926E−18 |
| Capzb | 0.449426755 | 10.36936752 | 85.10253926 | 2.83287E−20 | 4.18617E−18 |
| Arpc2 | 0.346828272 | 10.81151028 | 84.90559109 | 3.12957E−20 | 4.57489E−18 |
| Cd48 | 0.616642092 | 10.0606017 | 83.06747655 | 7.92997E−20 | 1.14689E−17 |
| Clic1 | 0.426604672 | 10.51946677 | 82.45485129 | 1.08112E−19 | 1.54713E−17 |
| Actn2 | 0.736387991 | 10.03167641 | 81.77982597 | 1.52123E−19 | 2.15429E−17 |
| Atp51 | 0.360702257 | 10.67458586 | 80.93617442 | 2.33127E−19 | 3.26738E−17 |
| Dedd2 | −0.731727354 | 9.632338182 | 79.43149123 | 4.9924E−19 | 6.92568E−17 |
| Ltb4r1 | 0.814916101 | 9.601858422 | 78.85441688 | 6.68598E−19 | 9.1814E−17 |
| Ctsw | 0.566396655 | 10.24368719 | 78.62204359 | 7.52055E−19 | 1.02242E−16 |
| Prelid1 | 0.656109752 | 9.891171439 | 78.22322584 | 9.20301E−19 | 1.23876E−16 |
| Tra2b | −0.488661709 | 10.19577356 | 78.08892868 | 9.85045E−19 | 1.31291E−16 |
| AA467197 | 0.877651163 | 9.704984918 | 77.24922157 | 1.50691E−18 | 1.98897E−16 |
| Pycard | 0.6838354 | 9.919490954 | 77.04275231 | 1.67297E−18 | 2.18693E−16 |
| Rac2 | 0.29382085 | 11.24349839 | 76.89574027 | 1.80225E−18 | 2.31148E−16 |
| Pim1 | −0.515291779 | 11.06070107 | 76.91273522 | 1.78681E−18 | 2.31148E−16 |
| H2-Q7 | −0.386384264 | 10.55176719 | 76.55241546 | 2.14445E−18 | 2.72465E−16 |
| Sdhb | 0.633252714 | 9.861109849 | 76.36407516 | 2.35904E−18 | 2.96955E−16 |
| Ctla4 | −0.596909051 | 10.20745116 | 75.50013879 | 3.65381E−18 | 4.55721E−16 |
| Clk1 | −0.544683385 | 10.07692244 | 75.47808739 | 3.69485E−18 | 4.56649E−16 |
| Gngt2 | −0.726299968 | 9.753641865 | 74.79477041 | 5.22279E−18 | 6.39674E−16 |
| Ltb | 0.532854208 | 10.5105756 | 74.55802359 | 5.8882E−18 | 7.14732E−16 |
| Nkg7 | 0.509213128 | 11.37225605 | 74.38601574 | 6.42425E−18 | 7.729E−16 |
| Maff | −0.79964788 | 9.685276468 | 73.36704807 | 1.07648E−17 | 1.28375E−15 |
| Uba52 | −0.358108045 | 10.48612483 | 72.43979314 | 1.72206E−17 | 2.03578E−15 |
| Cyb5a | 0.670808798 | 9.838120266 | 72.29714293 | 1.85115E−17 | 2.16952E−15 |
| Hmgb2 | 0.48219955 | 10.90748683 | 71.21488181 | 3.20363E−17 | 3.7225E−15 |
| Ccl5 | 0.756221662 | 13.88516502 | 71.19613316 | 3.23421E−17 | 3.7262E−15 |
| Gm9843 | −0.351089612 | 10.56964271 | 70.95037373 | 3.66324E−17 | 4.18502E−15 |
| Rbm39 | −0.311869482 | 10.84233663 | 70.52963247 | 4.53405E−17 | 5.1367E−15 |
| Myl12a | 0.366072373 | 10.71572431 | 70.13817584 | 5.52925E−17 | 6.21241E−15 |
| Dnajb4 | −0.770020972 | 9.617001768 | 70.05549857 | 5.76592E−17 | 6.42522E−15 |
| Gm11808 | −0.372654204 | 10.38068834 | 69.90975696 | 6.20806E−17 | 6.86167E−15 |
| Fyn | −0.550598359 | 9.891289908 | 69.7258551 | 6.8147E−17 | 7.47143E−15 |
| Actg1 | 0.324605067 | 11.94604458 | 69.47463064 | 7.74037E−17 | 8.41843E−15 |
| Itm2b | 0.314557643 | 11.12684555 | 69.30828715 | 8.4215E−17 | 9.08653E−15 |
| Ap2s1 | 0.644724334 | 9.796122343 | 68.94810314 | 1.01089E−16 | 1.08213E−14 |
| Ass1 | 0.738922713 | 9.844567328 | 68.21415472 | 1.46669E−16 | 1.55779E−14 |
| Btf3 | 0.303005054 | 10.81309008 | 67.9718735 | 1.65844E−16 | 1.74779E−14 |
| Glipr2 | 0.730000014 | 9.753269937 | 67.88661614 | 1.73173E−16 | 1.81099E−14 |
| Avl9 | −0.653986116 | 9.731535525 | 67.59763449 | 2.00508E−16 | 2.08084E−14 |
| Ccr9 | 0.832857974 | 9.857154544 | 66.4667878 | 3.55839E−16 | 3.66487E−14 |
| Phlda1 | −0.788431684 | 9.719239609 | 66.02764339 | 4.44643E−16 | 4.54505E−14 |
| Tcf7 | −0.644655858 | 9.792717352 | 65.67511056 | 5.31732E−16 | 5.39469E−14 |
| Selplg | 0.423459877 | 10.38010922 | 65.19224058 | 6.79365E−16 | 6.84146E−14 |
| mt-Atp8 | −0.67106869 | 9.60986082 | 65.17736304 | 6.84513E−16 | 6.84262E−14 |
| Rps15 | −0.216414105 | 11.50041078 | 64.92079989 | 7.79697E−16 | 7.73721E−14 |
| mt-Nd4 | −0.330338383 | 10.75014686 | 64.82286657 | 8.19427E−16 | 8.07254E−14 |
| Anxa2 | 0.722644648 | 9.881882506 | 64.73674853 | 8.56034E−16 | 8.3725E−14 |
| Cmtm7 | 0.647790672 | 9.807118552 | 64.60455779 | 9.15434E−16 | 8.88951E−14 |
| Tnfsf8 | 0.902577267 | 10.03565206 | 64.1198142 | 1.17078E−15 | 1.12885E−13 |
| Eno1 | 0.487848032 | 10.1319159 | 64.08709309 | 1.19039E−15 | 1.13967E−13 |
| Nfkbiz | −0.574034994 | 9.90564368 | 62.46141902 | 2.71716E−15 | 2.5832E−13 |
| Fxyd5 | 0.312416423 | 10.90172663 | 62.20646567 | 3.09271E−15 | 2.91982E−13 |
| Eif3h | 0.357810898 | 10.43681116 | 60.86483882 | 6.1131E−15 | 5.73156E−13 |
| Epsti1 | 0.463984054 | 10.26085662 | 60.42054667 | 7.66091E−15 | 7.13357E−13 |
| Cdc42 | 0.336991213 | 10.5652973 | 59.55545044 | 1.18896E−14 | 1.09959E−12 |
| Oas1a | −0.500430515 | 9.548339965 | 59.48066541 | 1.23502E−14 | 1.13446E−12 |
| Tox | −0.678206587 | 9.574160554 | 59.40704481 | 1.28209E−14 | 1.1698E−12 |
| Eif1 | −0.213223134 | 11.84549357 | 58.9703578 | 1.60065E−14 | 1.45072E−12 |
| Rsrp1 | 0.438310679 | 10.37261005 | 58.78113414 | 1.76223E−14 | 1.58659E−12 |
| Mbd2 | −0.588160192 | 9.73972323 | 58.59763888 | 1.93448E−14 | 1.73022E−12 |
| Sumo2 | 0.349177283 | 10.46272601 | 58.53783873 | 1.99419E−14 | 1.77196E−12 |
| Rpl13a | 0.155012279 | 13.10930771 | 58.46581604 | 2.06854E−14 | 1.82609E−12 |
| Tm2d2 | 0.70831406 | 9.63412223 | 58.26594334 | 2.28974E−14 | 2.00832E−12 |
| Fam101b | −0.645014148 | 9.557985731 | 58.10375522 | 2.48651E−14 | 2.16693E−12 |
| Cotl1 | 0.387933321 | 10.63154185 | 57.52288348 | 3.3407E−14 | 2.87448E−12 |
| Anxa6 | 0.529264421 | 9.947806186 | 57.53218843 | 3.32493E−14 | 2.87448E−12 |
| Ppp1ca | 0.358071833 | 10.45650421 | 57.42631271 | 3.50882E−14 | 3.00015E−12 |
| Lat | 0.30090055 | 10.83480524 | 57.38755335 | 3.57865E−14 | 3.04073E−12 |
| Dusp10 | −0.701807188 | 9.734497108 | 57.23375587 | 3.86972E−14 | 3.26763E−12 |
| Plac8 | 0.676955079 | 10.50997571 | 56.88041406 | 4.63136E−14 | 3.88662E−12 |
| Rasgrp1 | −0.606007374 | 9.755482259 | 56.82249163 | 4.7698E−14 | 3.97824E−12 |
| Ostf1 | 0.446931969 | 10.18723338 | 56.7994366 | 4.82605E−14 | 4.00062E−12 |
| Coro1a | 0.296036382 | 11.05941162 | 56.57479441 | 5.41009E−14 | 4.45759E−12 |
| 3830403N18Rik | 0.585483615 | 9.565458172 | 56.42066917 | 5.85121E−14 | 4.792E−12 |
| Lncpint | −0.683465689 | 9.609082259 | 56.32648915 | 6.13829E−14 | 4.99701E−12 |
| Uhrf2 | −0.636453151 | 9.805742233 | 56.23935181 | 6.41644E−14 | 5.19235E−12 |
| Slc5a3 | −0.573447632 | 9.553448168 | 56.14658929 | 6.72642E−14 | 5.41099E−12 |
| Dnajc15 | 0.456892242 | 10.09941767 | 55.77454562 | 8.12771E−14 | 6.49978E−12 |
| Rpsa | 0.216885157 | 11.60513144 | 55.21856755 | 1.07845E−13 | 8.57401E−12 |
| Arpc1b | 0.273302652 | 10.796252 | 55.10881601 | 1.14038E−13 | 9.01366E−12 |
| Cxcr5 | −0.518113656 | 9.543138573 | 54.75402302 | 1.36599E−13 | 1.07345E−11 |
| Klf4 | −0.752577219 | 9.566058556 | 54.6749753 | 1.42206E−13 | 1.11108E−11 |
| Rpl4 | 0.194418668 | 11.72659741 | 54.3921545 | 1.64217E−13 | 1.27573E−11 |
| Ifngr1 | 0.390565787 | 10.81929358 | 54.24193902 | 1.77263E−13 | 1.36925E−11 |
| Ifng | −0.868111197 | 9.899362081 | 54.09247489 | 1.91272E−13 | 1.46802E−11 |
| Rps6 | 0.165515494 | 12.27313172 | 54.08288209 | 1.92208E−13 | 1.46802E−11 |
| Rhoh | −0.534449576 | 9.837142269 | 54.02000635 | 1.98459E−13 | 1.50729E−11 |
| Cfl1 | 0.250192333 | 11.29422153 | 53.90728076 | 2.10177E−13 | 1.58742E−11 |
| Sptssa | 0.543646713 | 9.96326749 | 53.71520935 | 2.31762E−13 | 1.74078E−11 |
| mt-Cytb | −0.262082792 | 11.3111964 | 53.61065466 | 2.4443E−13 | 1.82584E−11 |
| Rps16 | −0.163309899 | 12.29143504 | 53.35615702 | 2.78238E−13 | 2.06702E−11 |
| Bcl2 | −0.704176859 | 9.768025777 | 52.18045653 | 5.06275E−13 | 3.74066E−11 |
| Txk | −0.607016555 | 9.712272046 | 52.10336758 | 5.26546E−13 | 3.8694E−11 |
| Acp5 | 0.578040126 | 9.78903337 | 51.61203372 | 6.76258E−13 | 4.94287E−11 |
| Tspo | 0.428503603 | 10.1185085 | 51.38616936 | 7.58712E−13 | 5.51588E−11 |
| Ccl20 | −0.810411094 | 9.550654384 | 51.08543215 | 8.84322E−13 | 6.39487E−11 |
| Rpl22l1 | 0.261648578 | 11.13468717 | 50.8848051 | 9.79489E−13 | 7.04558E−11 |
| Ywhah | 0.468443163 | 10.00113273 | 50.82097896 | 1.01186E−12 | 7.24015E−11 |
| Srrm2 | −0.411464931 | 10.16628193 | 50.49824308 | 1.19272E−12 | 8.45462E−11 |
| Cox4i1 | 0.260656313 | 10.85377808 | 50.49607584 | 1.19403E−12 | 8.45462E−11 |
| AW112010 | 0.291301825 | 11.71248698 | 50.43757707 | 1.23016E−12 | 8.6653E−11 |
| Pnrc1 | −0.326990414 | 10.78196761 | 50.29780369 | 1.32097E−12 | 9.25702E−11 |
| mt-Nd1 | −0.2653544 | 11.28494672 | 50.0634124 | 1.48857E−12 | 1.0378E−10 |
| Saraf | −0.365543292 | 10.30384933 | 49.9328995 | 1.59095E−12 | 1.10352E−10 |
| Ppib | 0.404822673 | 10.10011401 | 49.89019449 | 1.62595E−12 | 1.12207E−10 |
| Pgls | 0.555219426 | 9.786227624 | 49.5295049 | 1.95411E−12 | 1.34172E−10 |
| Dusp2 | 0.724800828 | 9.811340529 | 49.34390873 | 2.148E−12 | 1.46744E−10 |
| Plp2 | 0.604877901 | 9.591056513 | 49.25934175 | 2.24263E−12 | 1.52443E−10 |
| Chst12 | 0.650249975 | 9.626540055 | 48.68536907 | 3.005E−12 | 2.03249E−10 |
| Ldha | 0.363117284 | 10.40180441 | 48.62626572 | 3.09694E−12 | 2.0843E−10 |
| Fcer1g | −0.360864211 | 9.537643508 | 48.33462077 | 3.59351E−12 | 2.40659E−10 |
| Actr3 | 0.317519138 | 10.50564793 | 47.9423529 | 4.38937E−12 | 2.92517E−10 |
| Lpxn | 0.464741167 | 9.978535981 | 47.92799909 | 4.42162E−12 | 2.93229E−10 |
| Ybx3 | 0.60013217 | 9.599423261 | 47.61601782 | 5.18432E−12 | 3.4214E−10 |
| mt-Nd4l | −0.576765365 | 9.612318145 | 47.30905404 | 6.06315E−12 | 3.98163E−10 |
| Ccnl1 | −0.505607902 | 9.806469989 | 47.29981488 | 6.0918E−12 | 3.98163E−10 |
| Tmed2 | 0.388398507 | 10.11413497 | 47.23996875 | 6.28066E−12 | 4.08543E−10 |
| Hnrnpk | 0.324953239 | 10.40058528 | 46.9167236 | 7.4068E−12 | 4.79502E−10 |
| Gpr171 | 0.492019928 | 9.992435057 | 46.74373012 | 8.09034E−12 | 5.21271E−10 |
| Ifi27 | −0.48213806 | 9.837918078 | 46.66378408 | 8.42721E−12 | 5.40415E−10 |
| Rpl8 | 0.187142948 | 11.68698224 | 46.50058276 | 9.15912E−12 | 5.84593E−10 |
| Furin | −0.58250301 | 10.5517053 | 46.2400125 | 1.04618E−11 | 6.64621E−10 |
| Chd3 | −0.531844197 | 9.761077098 | 46.22174406 | 1.05599E−11 | 6.67726E−10 |
| Socs3 | −0.606438847 | 9.77952663 | 45.945977 | 1.21559E−11 | 7.65092E−10 |
| Klrd1 | 0.740622758 | 9.641291475 | 45.83007932 | 1.28968E−11 | 8.07981E−10 |
| Atp5f1 | 0.407703841 | 10.0510207 | 45.80329176 | 1.30744E−11 | 8.15349E−10 |
| mt-Co1 | 0.185405435 | 12.35512272 | 44.9562193 | 2.01489E−11 | 1.2508E−09 |
| Vasp | 0.352930373 | 10.31326066 | 44.93521323 | 2.03662E−11 | 1.25854E−09 |
| Mxd1 | −0.513254014 | 9.919607654 | 44.40709369 | 2.66719E−11 | 1.64074E−09 |
| Lgals3 | 0.683541289 | 10.03825331 | 44.0922441 | 3.1326E−11 | 1.91836E−09 |
| Sh2d1a | −0.486118075 | 9.853138169 | 44.01761352 | 3.25434E−11 | 1.98398E−09 |
| Srsf5 | −0.338591946 | 10.31310722 | 43.5233511 | 4.1893E−11 | 2.54257E−09 |
| Ywhae | 0.464501426 | 9.922516842 | 43.45147215 | 4.34605E−11 | 2.61436E−09 |
| Adam8 | 0.535877549 | 9.576468918 | 43.45269502 | 4.34333E−11 | 2.61436E−09 |
| Hmgb1 | 0.323083852 | 10.55704335 | 43.41280646 | 4.43278E−11 | 2.65478E−09 |
| Cisd3 | 0.602086754 | 9.632969394 | 43.28209301 | 4.73901E−11 | 2.82574E−09 |
| Ccdc117 | −0.570173683 | 9.594031621 | 42.87429725 | 5.83726E−11 | 3.46539E−09 |
| Ubald2 | −0.36571417 | 10.66143541 | 42.56689302 | 6.83058E−11 | 4.03747E−09 |
| Tram1 | 0.459368816 | 9.883728186 | 42.3939177 | 7.46217E−11 | 4.3917E−09 |
| Ddx60 | −0.373678564 | 9.541837476 | 41.90575092 | 9.57805E−11 | 5.61266E−09 |
| Rpl18 | 0.16500821 | 11.86839686 | 41.52080501 | 1.16623E−10 | 6.80469E−09 |
| Ftl1 | −0.254126258 | 11.02867874 | 41.24955394 | 1.33982E−10 | 7.78415E−09 |
| Cnn2 | 0.351443965 | 10.25976365 | 41.23898797 | 1.34709E−10 | 7.79304E−09 |
| Arf5 | 0.335902191 | 10.19854297 | 41.17413621 | 1.39253E−10 | 8.0218E−09 |
| Rps28 | −0.17669445 | 11.84765533 | 41.03874747 | 1.49241E−10 | 8.56089E−09 |
| Nrip1 | −0.533322514 | 9.859993551 | 40.99470604 | 1.52642E−10 | 8.7192E−09 |
| Uqcrfs1 | 0.407154567 | 9.966955579 | 40.84086269 | 1.65144E−10 | 9.3547E−09 |
| Ddx24 | −0.456807659 | 9.828797982 | 40.84178616 | 1.65066E−10 | 9.3547E−09 |
| Spcs3 | 0.558898275 | 9.669879283 | 40.41720553 | 2.05127E−10 | 1.15714E−08 |
| Psmd13 | 0.539440109 | 9.697470476 | 40.40366824 | 2.06553E−10 | 1.16037E−08 |
| Sdcbp | 0.448461817 | 9.891466083 | 40.3625637 | 2.10945E−10 | 1.18016E−08 |
| Rtp4 | −0.563163546 | 9.565691325 | 39.98728135 | 2.55622E−10 | 1.42425E−08 |
| Cd28 | −0.364314583 | 10.50430705 | 39.97688609 | 2.56986E−10 | 1.42601E−08 |
| Gm10076 | −0.363789063 | 10.01444869 | 39.8563226 | 2.73348E−10 | 1.51064E−08 |
| 1500009L16Rik | 0.542202591 | 9.583684757 | 39.7287938 | 2.91792E−10 | 1.60604E−08 |
| S100a13 | 0.386023024 | 10.11272451 | 39.59415967 | 3.12617E−10 | 1.71372E−08 |
| Tprgl | 0.439315767 | 9.937270565 | 39.49031889 | 3.2969E−10 | 1.80005E−08 |
| Uqcrh | 0.258509129 | 10.66055691 | 39.26239503 | 3.70509E−10 | 2.01483E−08 |
| Hnrnpdl | −0.425883748 | 9.851559277 | 39.24467677 | 3.73886E−10 | 2.02509E−08 |
| Trps1 | −0.581094547 | 9.593491566 | 39.22474925 | 3.77722E−10 | 2.03775E−08 |
| Sh3bgrl3 | 0.234852888 | 11.18097545 | 39.21492611 | 3.79627E−10 | 2.03993E−08 |
| Nr4a1 | −0.490052945 | 9.985650751 | 39.19056878 | 3.84393E−10 | 2.05741E−08 |
| Zfand2a | −0.537861078 | 9.566631043 | 39.02336362 | 4.18764E−10 | 2.23259E−08 |
| Irf1 | −0.514113223 | 9.768522493 | 38.96928106 | 4.30527E−10 | 2.28633E−08 |
| Limd2 | 0.322761237 | 10.43727909 | 38.93205538 | 4.38815E−10 | 2.31229E−08 |
| Zbtb20 | −0.581062467 | 9.584425294 | 38.93617746 | 4.3789E−10 | 2.31229E−08 |
| Asb2 | −0.544248262 | 9.850342678 | 38.84496818 | 4.58835E−10 | 2.40844E−08 |
| Tiparp | −0.580123163 | 9.609123279 | 38.4953443 | 5.48845E−10 | 2.86982E−08 |
| Isg15 | −0.584150245 | 9.800573377 | 38.45870296 | 5.59246E−10 | 2.91301E−08 |
| Csrnp1 | −0.517948042 | 9.779414893 | 38.26534917 | 6.17494E−10 | 3.20414E−08 |
| Tmem50a | 0.386836955 | 10.00814313 | 38.15703896 | 6.52738E−10 | 3.37414E−08 |
| Actn1 | −0.533996189 | 9.567435546 | 38.08192888 | 6.78355E−10 | 3.49327E−08 |
| Psmd8 | 0.424531567 | 9.892382532 | 38.00538221 | 7.05497E−10 | 3.60573E−08 |
| Gm26699 | −0.473480345 | 9.765120668 | 38.00550982 | 7.05451E−10 | 3.60573E−08 |
| Cd244 | 0.388278993 | 9.55144543 | 37.91921304 | 7.37355E−10 | 3.75444E−08 |
| Rps11 | 0.141420944 | 12.35636328 | 37.86091045 | 7.59723E−10 | 3.85173E−08 |
| Ilk | 0.536823666 | 9.680259126 | 37.85474135 | 7.62129E−10 | 3.85173E−08 |
| Sdcbp2 | −0.542086538 | 9.622445807 | 37.59342632 | 8.71377E−10 | 4.38754E−08 |
| Ahr | −0.59092619 | 9.672948429 | 37.54436013 | 8.93574E−10 | 4.48271E−08 |
| Rps21 | −0.21136896 | 10.97596554 | 37.1282141 | 1.10612E−09 | 5.52855E−08 |
| Plin2 | −0.537264515 | 9.575959069 | 37.10475461 | 1.1195E−09 | 5.57497E−08 |
| Rpl10a | 0.200698396 | 11.2570968 | 37.0300252 | 1.16324E−09 | 5.77163E−08 |
| Sdf2l1 | 0.529962562 | 9.709620662 | 37.00327913 | 1.17931E−09 | 5.83007E−08 |
| Cox5b | 0.305999662 | 10.32202341 | 36.9673266 | 1.20125E−09 | 5.91705E−08 |
| Apobec3 | 0.437401019 | 9.902621042 | 36.93631025 | 1.22052E−09 | 5.99022E−08 |
| Rpl9-ps6 | −0.351320591 | 9.989161867 | 36.53264344 | 1.50131E−09 | 7.34183E−08 |
| Gem | −0.682793999 | 9.688105676 | 36.46631611 | 1.55327E−09 | 7.56873E−08 |
| Eprs | 0.53788575 | 9.694503208 | 36.43986079 | 1.5745E−09 | 7.64476E−08 |
| Gstp1 | −0.504141129 | 9.64657452 | 36.38114233 | 1.62266E−09 | 7.85054E−08 |
| Trp53inp1 | −0.513550556 | 9.595006556 | 36.2425247 | 1.74227E−09 | 8.39935E−08 |
| Ahnak | 0.498211225 | 9.785411173 | 36.12837239 | 1.84737E−09 | 8.87458E−08 |
| Arpc4 | 0.366807267 | 10.06345714 | 36.05320569 | 1.92003E−09 | 9.19111E−08 |
| Rap1b | 0.315806112 | 10.27367424 | 36.01786076 | 1.95517E−09 | 9.32651E−08 |
| Kcnk5 | −0.515305339 | 9.574931238 | 35.71192387 | 2.2876E−09 | 1.08741E−07 |
| Cd9 | −0.471749722 | 9.565476632 | 35.54177757 | 2.49639E−09 | 1.18252E−07 |
| Ostc | 0.453743376 | 9.79984885 | 35.34117534 | 2.7672E−09 | 1.30625E−07 |
| Gnai2 | 0.271442641 | 10.46001918 | 35.19685572 | 2.98004E−09 | 1.40186E−07 |
| Npm3 | 0.426799674 | 9.885664227 | 34.91904872 | 3.43702E−09 | 1.61125E−07 |
| Hif1a | −0.430822834 | 9.908262886 | 34.69387483 | 3.85844E−09 | 1.80259E−07 |
| Spcs2 | 0.323040264 | 10.17009625 | 34.61830814 | 4.01116E−09 | 1.86752E−07 |
| Ap2m1 | 0.423344592 | 9.842161501 | 34.58702108 | 4.07615E−09 | 1.89101E−07 |
| Edf1 | 0.331625317 | 10.10367538 | 34.58068966 | 4.08943E−09 | 1.89101E−07 |
| Izumo1r | −0.621017876 | 9.606866954 | 34.41958125 | 4.44231E−09 | 2.0462E−07 |
| Manf | 0.417472957 | 9.905200221 | 34.41397814 | 4.45512E−09 | 2.0462E−07 |
| Tgfb1 | 0.456071377 | 9.908533916 | 34.35865156 | 4.58358E−09 | 2.09811E−07 |
| Rps3 | 0.136732909 | 11.97017084 | 34.23146089 | 4.89315E−09 | 2.22666E−07 |
| Degs1 | 0.499847178 | 9.710625605 | 34.22985792 | 4.89718E−09 | 2.22666E−07 |
| Ciapin1 | 0.505447675 | 9.69209499 | 33.8265408 | 6.02513E−09 | 2.73039E−07 |
| Btg2 | 0.338891136 | 10.81509287 | 33.75955166 | 6.23621E−09 | 2.81665E−07 |
| Hint1 | 0.319099291 | 10.22665553 | 33.64099662 | 6.6281E−09 | 2.9711E−07 |
| Casp4 | −0.476037532 | 9.726331048 | 33.63641218 | 6.64373E−09 | 2.9711E−07 |
| Anxa11 | 0.487304216 | 9.703047741 | 33.64158832 | 6.62608E−09 | 2.9711E−07 |
| Rnf115 | 0.477692622 | 9.719266495 | 33.60418158 | 6.75474E−09 | 3.01084E−07 |
| Coq10b | −0.435536512 | 9.823130381 | 33.38844922 | 7.54709E−09 | 3.35303E−07 |
| Gltscr2 | 0.281894891 | 10.46438057 | 33.12678111 | 8.63413E−09 | 3.82348E−07 |
| Ero1l | 0.519056603 | 9.611552364 | 33.00007472 | 9.21553E−09 | 4.0677E−07 |
| Tomm7 | −0.313476679 | 10.05823488 | 32.94049529 | 9.50232E−09 | 4.18071E−07 |
| Ywhaq | 0.413376988 | 9.880284734 | 32.91496101 | 9.62795E−09 | 4.22232E−07 |
| Psmb8 | 0.247937082 | 10.58696086 | 32.69682093 | 1.07713E−08 | 4.70856E−07 |
| Rhob | −0.466893188 | 9.565551549 | 32.68298283 | 1.08483E−08 | 4.727E−07 |
| Rps10 | 0.169459818 | 11.27074258 | 32.67170702 | 1.09114E−08 | 4.73931E−07 |
| Cd82 | 0.343430004 | 10.23707609 | 32.59801177 | 1.13331E−08 | 4.90678E−07 |
| Abcb1a | −0.490964544 | 9.634618666 | 32.5330167 | 1.17185E−08 | 5.05754E−07 |
| Ecm1 | −0.428948374 | 9.548168048 | 32.5121924 | 1.18447E−08 | 5.09585E−07 |
| Chchd2 | −0.242250124 | 10.4145197 | 32.4935448 | 1.19589E−08 | 5.12875E−07 |
| Stmn2 | 0.31474375 | 9.547934398 | 32.44033935 | 1.22908E−08 | 5.25453E−07 |
| Ptprk | 0.322461174 | 9.547124172 | 32.39925874 | 1.25534E−08 | 5.34997E−07 |
| Zfp280d | −0.455933504 | 9.577105785 | 32.39298477 | 1.2594E−08 | 5.3505E−07 |
| RP24-48411.3 | 0.45581967 | 9.573621121 | 32.28892999 | 1.32868E−08 | 5.62724E−07 |
| Vps37b | −0.330737492 | 10.53077373 | 32.22402668 | 1.37381E−08 | 5.80031E−07 |
| Tuba4a | −0.495588026 | 9.695802967 | 32.05135878 | 1.5015E−08 | 6.31978E−07 |
| Cd27 | −0.623155836 | 9.654241658 | 31.96170218 | 1.57242E−08 | 6.59787E−07 |
| Rps19 | 0.147463744 | 12.62869486 | 31.76897993 | 1.73644E−08 | 7.26366E−07 |
| Ndufa11 | 0.395844467 | 9.869001574 | 31.75589903 | 1.74817E−08 | 7.29032E−07 |
| Pglyrp1 | 0.554448261 | 9.675823263 | 31.7328135 | 1.76908E−08 | 7.35493E−07 |
| Gpr132 | −0.347377843 | 10.08111363 | 31.62652046 | 1.8686E−08 | 7.74501E−07 |
| Dnajb2 | −0.398965682 | 9.552710551 | 31.53488229 | 1.9589E−08 | 8.09458E−07 |
| Sfr1 | 0.436841829 | 9.79905978 | 31.52744207 | 1.96642E−08 | 8.10103E−07 |
| Stmn1 | 0.869255435 | 9.718320163 | 31.23884134 | 2.28155E−08 | 9.3709E−07 |
| Chn2 | 0.50726472 | 9.602283449 | 31.11036095 | 2.43767E−08 | 9.98195E−07 |
| Atp8a2 | 0.515838144 | 9.630965479 | 30.97464046 | 2.61422E−08 | 1.06728E−06 |
| Cd40lg | −0.471028099 | 9.806462091 | 30.89285541 | 2.72674E−08 | 1.10895E−06 |
| Cxcl10 | −0.727462227 | 9.649729541 | 30.88867223 | 2.73262E−08 | 1.10895E−06 |
| Egr1 | −0.58434076 | 9.573320341 | 30.82712287 | 2.82068E−08 | 1.14128E−06 |
| Nubp1 | 0.473403814 | 9.680677072 | 30.783805 | 2.88435E−08 | 1.16358E−06 |
| Eif3m | 0.33858974 | 10.03086842 | 30.533751 | 3.28108E−08 | 1.31971E−06 |
| Gm26917 | −0.386612599 | 9.867968155 | 30.27290912 | 3.75332E−08 | 1.5052E−06 |
| Atp5a1 | 0.330745474 | 10.05398237 | 30.25219304 | 3.79362E−08 | 1.51689E−06 |
| Sytl1 | 0.450310542 | 9.57672802 | 30.16768361 | 3.96258E−08 | 1.5798E−06 |
| Mier1 | 0.429759379 | 9.849073912 | 29.99380834 | 4.33428E−08 | 1.71869E−06 |
| Tagap | −0.481023723 | 9.753295815 | 29.99293376 | 4.33624E−08 | 1.71869E−06 |
| Ahsa1 | −0.415991957 | 9.7343473 | 29.8494272 | 4.66935E−08 | 1.84534E−06 |
| Acot9 | 0.486670609 | 9.629157206 | 29.79529145 | 4.80157E−08 | 1.8921E−06 |
| Rps3a1 | 0.144785051 | 11.73486958 | 29.76024635 | 4.88915E−08 | 1.92104E−06 |
| Serinc3 | 0.488696277 | 9.653334653 | 29.75265681 | 4.90833E−08 | 1.92302E−06 |
| Rplp2 | −0.134128458 | 11.87909328 | 29.57476782 | 5.38008E−08 | 2.10179E−06 |
| Eif5a | 0.271331035 | 10.45412985 | 29.306058 | 6.18022E−08 | 2.40745E−06 |
| Tmem176a | 0.659501681 | 9.760786899 | 29.24814254 | 6.36772E−08 | 2.47341E−06 |
| Sh2d2a | 0.372695089 | 10.07434142 | 29.15725241 | 6.67355E−08 | 2.58481E−06 |
| Prmt1 | 0.433541135 | 9.752599757 | 29.09939463 | 6.87585E−08 | 2.64808E−06 |
| Wdr89 | −0.260435474 | 10.29803674 | 29.10467471 | 6.85713E−08 | 2.64808E−06 |
| Msn | 0.303992139 | 10.11460226 | 28.9457299 | 7.44347E−08 | 2.85859E−06 |
| Ict1 | 0.436674726 | 9.753338871 | 28.93128095 | 7.4992E−08 | 2.87188E−06 |
| Ythdc1 | −0.434898908 | 9.695559663 | 28.82288915 | 7.93083E−08 | 3.02864E−06 |
| Rpl13-ps3 | −0.319707654 | 9.989340753 | 28.73260083 | 8.30931E−08 | 3.16429E−06 |
| Gimap6 | −0.244548357 | 10.59710866 | 28.72414041 | 8.34569E−08 | 3.16926E−06 |
| Tmem30a | 0.437623733 | 9.744492786 | 28.69389532 | 8.47705E−08 | 3.21018E−06 |
| Rac1 | 0.311594293 | 10.06053352 | 28.63136373 | 8.75526E−08 | 3.29865E−06 |
| Sesn1 | −0.368525972 | 9.551406608 | 28.63049009 | 8.75921E−08 | 3.29865E−06 |
| 4930503L19Rik | −0.444136554 | 9.569307661 | 28.62410095 | 8.78815E−08 | 3.30041E−06 |
| Gramd3 | 0.454475792 | 10.22334111 | 28.60513397 | 8.87466E−08 | 3.32372E−06 |
| Nt5c | 0.418853625 | 9.783355854 | 28.57256775 | 9.02518E−08 | 3.3708E−06 |
| Zyx | 0.49949057 | 9.653615873 | 28.51246827 | 9.3097E−08 | 3.46755E−06 |
| Jmjd1c | −0.470611575 | 9.671839485 | 28.41495083 | 9.79063E−08 | 3.63671E−06 |
| Oaz1 | 0.157157942 | 11.32703266 | 28.22495552 | 1.08003E−07 | 4.00082E−06 |
| Atp5o | 0.340449858 | 9.966789533 | 28.09237029 | 1.15661E−07 | 4.27285E−06 |
| Slc25a4 | 0.403613648 | 9.854301686 | 28.07715311 | 1.16574E−07 | 4.29491E−06 |
| 2810417H13Rik | 0.864238455 | 9.675098575 | 28.05895758 | 1.17675E−07 | 4.32377E−06 |
| Cmpk1 | 0.392640439 | 9.806513171 | 27.94059504 | 1.25098E−07 | 4.5841E−06 |
| Stat3 | −0.40036914 | 9.799207752 | 27.92008019 | 1.26431E−07 | 4.6205E−06 |
| Tmem160 | 0.361394762 | 9.896444569 | 27.89373698 | 1.28164E−07 | 4.67128E−06 |
| Atf4 | −0.416309212 | 9.732097606 | 27.83823326 | 1.31894E−07 | 4.79437E−06 |
| Zfp706 | 0.377304609 | 9.852188098 | 27.77685472 | 1.36145E−07 | 4.93572E−06 |
| Prr13 | 0.259162854 | 10.44540802 | 27.68287373 | 1.42922E−07 | 5.16764E−06 |

TABLE 2D
Colon IEL Ctrl vs KO

| GeneSymbol | logFC | logCPM | LR | PValue | FDR |
|---|---|---|---|---|---|
| Hspa1a | −4.912418231 | 10.69640911 | 4353.406085 | 0 | 0 |
| Ubb | −0.77403293 | 12.42365996 | 2268.704696 | 0 | 0 |
| Malat1 | −0.969266612 | 14.67682077 | 2468.851731 | 0 | 0 |
| Rbm3 | 0.950879034 | 10.81487562 | 1615.458299 | 0 | 0 |
| Dnajb1 | −2.106865401 | 9.893638618 | 1434.569266 | 0 | 0 |
| Jun | −2.095847257 | 10.05710788 | 1333.458514 | 6.0574E−292 | 1.4803E−288 |
| Fos | −2.354918413 | 10.11294467 | 1287.060873 | 7.3297E−282 | 1.5354E−278 |
| Ubc | −0.885778987 | 10.8737827 | 1160.271008 | 2.6281E−254 | 4.8169E−251 |
| Hspa1b | −1.572519816 | 9.464180989 | 1005.757106 | 1.0067E−220 | 1.6401E−217 |
| Rps27rt | −0.855944525 | 10.45058647 | 960.5894229 | 6.6203E−211 | 9.7073E−208 |
| Rps27 | −0.456664488 | 12.77091707 | 884.4000389 | 2.416E−194 | 3.2205E−191 |
| Rsrp1 | −0.7538665 | 10.47574847 | 825.1217025 | 1.8632E−181 | 2.2767E−178 |
| Rpl38 | −0.450854758 | 11.36672195 | 727.8453794 | 2.6346E−160 | 2.9716E−157 |
| S100a4 | 1.196877127 | 10.20124983 | 675.3497526 | 6.858E−149 | 7.1828E−146 |
| Hsp90ab1 | −0.625671337 | 10.70244953 | 672.3653519 | 3.0564E−148 | 2.9878E−145 |
| Rplp0 | 0.358265483 | 12.22246018 | 637.178858 | 1.3725E−140 | 1.2578E−137 |
| Rpl37a | −0.341973881 | 12.31974039 | 618.461248 | 1.616E−136 | 1.3938E−133 |
| Asb2 | −1.087110199 | 9.986148994 | 587.987702 | 6.8646E−130 | 5.592E−127 |
| Dusp2 | 1.140087471 | 10.1241259 | 564.8664371 | 7.3454E−125 | 5.6687E−122 |
| Hsp90aa1 | −0.884650897 | 10.03922356 | 545.7934307 | 1.0354E−120 | 7.5908E−118 |
| Uba52 | −0.715686894 | 10.02413792 | 535.1932376 | 2.0948E−118 | 1.4627E−115 |
| Rps29 | −0.3494107 | 11.76226086 | 526.5778531 | 1.5684E−116 | 1.0453E−113 |
| S100a6 | 0.665033272 | 11.40505169 | 473.014171 | 7.0773E−105 | 4.5119E−102 |
| Rpl22l1 | 0.492941427 | 10.81346165 | 469.0919932 | 5.0508E−104 | 3.0858E−101 |
| Slc25a5 | 0.48478658 | 10.63486828 | 453.0258552 | 1.5835E−100 | 9.28738E−98 |
| Ass1 | 1.043502352 | 9.681786921 | 439.9535277 | 1.10805E−97 | 6.24899E−95 |
| Rpl32 | 0.270960395 | 12.3093303 | 421.5380521 | 1.12894E−93 | 6.131E−91 |
| Tmsb10 | −0.379217961 | 12.59401337 | 403.7190476 | 8.53796E−90 | 4.47115E−87 |
| Dynll1 | 0.539955191 | 10.37288347 | 403.0311001 | 1.20534E−89 | 6.09443E−87 |
| Rps28 | −0.361971479 | 11.20834805 | 399.8352948 | 5.98121E−89 | 2.92342E−86 |
| Rpl3 | 0.251619694 | 12.09990026 | 380.8827502 | 7.99417E−85 | 3.78124E−82 |
| H2-K1 | −0.345378791 | 11.57577177 | 372.4393757 | 5.50898E−83 | 2.52432E−80 |
| H2-D1 | −0.260139164 | 12.2312739 | 355.6894705 | 2.44458E−79 | 1.08621E−76 |
| Rpl29 | 0.374981802 | 11.0435012 | 354.0148476 | 5.66062E−79 | 2.44122E−76 |
| Mptx1 | −0.940597371 | 9.428866314 | 349.7728959 | 4.74892E−78 | 1.95256E−75 |
| Gngt2 | −0.855819774 | 9.686351451 | 349.7541182 | 4.79384E−78 | 1.95256E−75 |
| Gm10076 | −0.737584834 | 9.708049893 | 340.536643 | 4.87528E−76 | 1.93206E−73 |
| Fosb | −0.857299353 | 9.419033424 | 337.6702214 | 2.05239E−75 | 7.91953E−73 |
| H2-Q7 | −0.485711612 | 10.45033154 | 337.4095826 | 2.33896E−75 | 8.79391E−73 |
| Rpl24 | 0.274190325 | 11.71749233 | 330.3088265 | 8.23237E−74 | 3.01778E−71 |
| Rpl41 | −0.209689141 | 12.53198319 | 315.3142841 | 1.51906E−70 | 5.43268E−68 |
| Plac8 | 0.788837939 | 10.43214592 | 307.2290402 | 8.76735E−69 | 3.06085E−66 |
| Rps2 | 0.31789432 | 11.392 | 298.9635462 | 5.54091E−67 | 1.88945E−64 |
| Klf6 | −0.788289496 | 10.16162079 | 294.3554236 | 5.59207E−66 | 1.86356E−63 |
| BC021614 | 0.881668333 | 9.536127554 | 287.8216032 | 1.48325E−64 | 4.8331E−62 |
| Stat1 | −0.701927733 | 9.831890541 | 283.3313126 | 1.41143E−63 | 4.4991E−61 |
| Ifngr1 | 0.611436865 | 10.15474423 | 279.8164944 | 8.23351E−63 | 2.56868E−60 |
| Crip1 | 0.445133772 | 11.63845182 | 278.3682928 | 1.70285E−62 | 5.20186E−60 |
| B2m | −0.192178232 | 12.76962568 | 273.0340514 | 2.47551E−61 | 7.40783E−59 |
| Rpsa | 0.27943496 | 11.44702036 | 271.2401369 | 6.09017E−61 | 1.786E−58 |
| Gm11808 | −0.543144555 | 9.925523693 | 269.4920183 | 1.46427E−60 | 4.20993E−58 |
| Eif4a2 | −0.603016602 | 9.902866294 | 267.009303 | 5.09016E−60 | 1.43533E−57 |
| Tomm7 | −0.49876914 | 9.956780815 | 256.1536398 | 1.18292E−57 | 3.27266E−55 |
| Vim | 0.478069679 | 10.67155362 | 255.4815988 | 1.6575E−57 | 4.50071E−55 |
| Rps3a1 | 0.242880798 | 11.51721161 | 254.1602609 | 3.21732E−57 | 8.57738E−55 |
| Rps11 | 0.21510739 | 12.18975268 | 245.7818357 | 2.15808E−55 | 5.65071E−53 |
| Cox17 | −0.476013244 | 10.03899143 | 243.7133207 | 6.09623E−55 | 1.56823E−52 |
| Rpl8 | 0.248159642 | 11.41010456 | 240.8276921 | 2.59557E−54 | 6.56187E−52 |
| Ptprcap | 0.389634275 | 10.51759166 | 240.350875 | 3.29761E−54 | 8.1954E−52 |
| Dusp1 | −0.904271717 | 10.0322272 | 236.1269017 | 2.74943E−53 | 6.71914E−51 |
| Pycard | 0.564371274 | 9.949662724 | 231.922578 | 2.27022E−52 | 5.45709E−50 |
| Bhlhe40 | 0.630615216 | 9.942561935 | 230.8478654 | 3.89438E−52 | 9.21022E−50 |
| Rcbtb2 | −0.742317781 | 9.655868727 | 229.5682165 | 7.4047E−52 | 1.72342E−49 |
| Rps6 | 0.203977864 | 12.02748398 | 229.4067809 | 8.02998E−52 | 1.83974E−49 |
| Rpl10a | 0.28962533 | 11.05684293 | 227.5062085 | 2.0855E−51 | 4.70456E−49 |
| Wdr89 | −0.524129877 | 9.860629364 | 225.9165989 | 4.63338E−51 | 1.02938E−48 |
| Ccnd2 | −0.468969455 | 10.33483348 | 218.2900005 | 2.13493E−49 | 4.67232E−47 |
| Rps21 | −0.33368489 | 10.56800568 | 217.5306651 | 3.12624E−49 | 6.74118E−47 |
| Eif3f | 0.304579066 | 10.76016509 | 214.3927099 | 1.51198E−48 | 3.21307E−46 |
| H2afz | 0.505010513 | 10.64921003 | 214.215217 | 1.65298E−48 | 3.46252E−46 |
| Gm9843 | −0.436865437 | 10.06758138 | 211.9041073 | 5.27779E−48 | 1.08998E−45 |
| Neat1 | 0.7478016 | 9.746628843 | 210.8010385 | 9.18551E−48 | 1.87066E−45 |
| Rpl18 | 0.221574798 | 11.68576023 | 209.1814055 | 2.07234E−47 | 4.16257E−45 |
| Ltb | 0.524512641 | 10.49312487 | 205.1159497 | 1.59766E−46 | 3.16575E−44 |
| Phlda1 | −0.955844714 | 9.533129244 | 202.73707 | 5.27912E−46 | 1.0321E−43 |
| Cd160 | 0.733852101 | 9.934715958 | 202.6916375 | 5.40102E−46 | 1.04204E−43 |
| Actg1 | 0.281414796 | 12.31487365 | 196.162142 | 1.43674E−44 | 2.73596E−42 |
| Eif2s3y | 0.687418848 | 9.547993406 | 195.8072505 | 1.71724E−44 | 3.22819E−42 |
| Satb1 | 0.751494377 | 9.529465747 | 194.8575828 | 2.76754E−44 | 5.13677E−42 |
| Serf2 | −0.250130527 | 10.9456737 | 193.9668336 | 4.33015E−44 | 7.93662E−42 |
| Ccl4 | 1.485785617 | 11.36531232 | 192.365257 | 9.68418E−44 | 1.75308E−41 |
| Ldha | 0.357711111 | 10.5986605 | 192.321415 | 9.89993E−44 | 1.77028E−41 |
| Ndufa3 | −0.395696872 | 10.08011666 | 190.6069001 | 2.34347E−43 | 4.14003E−41 |
| Jund | −0.387305885 | 11.36254002 | 189.980835 | 3.21007E−43 | 5.60348E−41 |
| Emp3 | 0.438130914 | 10.18357209 | 189.0713646 | 5.07031E−43 | 8.74659E−41 |
| Cd52 | 0.210775009 | 12.64363478 | 184.4061463 | 5.28984E−42 | 9.01918E−40 |
| Cd47 | 0.384229869 | 10.24153152 | 181.0529042 | 2.85451E−41 | 4.81101E−39 |
| Bola2 | −0.500931151 | 9.816144873 | 180.209206 | 4.36257E−41 | 7.26913E−39 |
| Nop10 | −0.447267852 | 9.964174868 | 178.3896367 | 1.08902E−40 | 1.79419E−38 |
| Cotl1 | 0.347861789 | 10.82474878 | 177.3790784 | 1.81008E−40 | 2.94901E−38 |
| Gpr18 | 0.707432613 | 9.62318615 | 176.8067404 | 2.41364E−40 | 3.88914E−38 |
| Cbx4 | −0.657383357 | 9.485214987 | 175.6581823 | 4.3001E−40 | 6.85351E−38 |
| Rps10 | 0.235850397 | 11.06031224 | 174.7426765 | 6.81396E−40 | 1.07433E−37 |
| Alyref | 0.562801095 | 9.80640343 | 173.2604322 | 1.4358E−39 | 2.2397E−37 |
| Hspe1 | −0.498929135 | 9.822812601 | 172.3436082 | 2.27677E−39 | 3.51413E−37 |
| Mif4gd | 0.596663991 | 9.624117642 | 171.0774302 | 4.30377E−39 | 6.57356E−37 |
| Btg1 | 0.33237651 | 11.54672282 | 170.0633984 | 7.16671E−39 | 1.08335E−36 |
| Ywhae | 0.451607278 | 9.886722446 | 169.5446464 | 9.30296E−39 | 1.39193E−36 |
| Fgl2 | −0.599214624 | 9.754853926 | 167.848361 | 2.18335E−38 | 3.23378E−36 |
| Abi3 | −0.514355959 | 9.839809501 | 166.8618241 | 3.58601E−38 | 5.25817E−36 |
| Cox7c | −0.295724359 | 10.44152118 | 166.0753922 | 5.32595E−38 | 7.73213E−36 |
| S100a11 | −0.296848696 | 10.91816565 | 165.5905177 | 6.79695E−38 | 9.77095E−36 |
| Coro1a | 0.233952804 | 11.24278869 | 161.3819745 | 5.64562E−37 | 8.03706E−35 |
| Zyx | 0.598281587 | 9.607741404 | 160.0838661 | 1.08474E−36 | 1.52938E−34 |
| Rad23a | −0.620382828 | 9.457741846 | 156.8432936 | 5.5385E−36 | 7.73439E−34 |
| Gmfg | 0.341067596 | 10.34860251 | 155.1988037 | 1.26692E−35 | 1.75253E−33 |
| Hmgb2 | 0.55422263 | 10.54001319 | 154.9366563 | 1.44556E−35 | 1.96262E−33 |
| Snrpg | −0.37093984 | 10.05500868 | 154.9375473 | 1.44491E−35 | 1.96262E−33 |
| Cd247 | −0.484714034 | 9.801664107 | 154.4549065 | 1.84211E−35 | 2.47806E−33 |
| Icos | 0.610190592 | 9.74486266 | 153.8284708 | 2.52473E−35 | 3.33515E−33 |
| Gapdh | 0.301102423 | 10.80087158 | 153.8412635 | 2.50853E−35 | 3.33515E−33 |
| Rgs10 | 0.478781426 | 9.963476703 | 153.4973144 | 2.98254E−35 | 3.90473E−33 |
| Lpar6 | −0.636355346 | 9.566478995 | 153.46167 | 3.03652E−35 | 3.94022E−33 |
| Klf2 | 0.990778098 | 10.07901749 | 150.7316392 | 1.19962E−34 | 1.54298E−32 |
| Ifi27 | −0.461697659 | 9.803250594 | 149.8692448 | 1.85157E−34 | 2.36083E−32 |
| H2afy | −0.325716736 | 10.24951783 | 147.3520485 | 6.57313E−34 | 8.30878E−32 |
| Rasgrp4 | −0.504364183 | 9.405509348 | 147.1171723 | 7.39804E−34 | 9.27158E−32 |
| Limd2 | 0.371058117 | 10.22214485 | 146.6145931 | 9.52761E−34 | 1.18393E−31 |
| mt-Co1 | 0.202675662 | 12.19348251 | 143.8546255 | 3.82274E−33 | 4.71032E−31 |
| Btf3 | 0.238036805 | 10.77125033 | 143.4716622 | 4.63559E−33 | 5.66431E−31 |
| Grcc10 | −0.537895935 | 9.60614827 | 143.4452179 | 4.69772E−33 | 5.69278E−31 |
| Lax1 | 0.604314275 | 9.491039638 | 142.4955928 | 7.57737E−33 | 9.10713E−31 |
| Tpm4 | 0.511683249 | 9.75042318 | 142.4364652 | 7.80633E−33 | 9.30603E−31 |
| Rpl4 | 0.175431416 | 11.61378607 | 142.0480365 | 9.49244E−33 | 1.12248E−30 |
| Gm9844 | −0.536348989 | 9.601478567 | 141.5180167 | 1.23957E−32 | 1.45406E−30 |
| Gm8797 | −0.579685824 | 9.452636417 | 138.8347619 | 4.78665E−32 | 5.57037E−30 |
| Txnip | −0.547016389 | 9.661178104 | 137.4469207 | 9.62828E−32 | 1.11165E−29 |
| Pglyrp1 | 0.644569275 | 9.565961199 | 135.9739899 | 2.02161E−31 | 2.31585E−29 |
| Gm42418 | −0.592774056 | 9.478911026 | 134.3456302 | 4.59061E−31 | 5.218E−29 |
| Ost4 | −0.422518077 | 9.806199001 | 133.7229779 | 6.28163E−31 | 7.08519E−29 |
| Atp5e | −0.277541227 | 10.36033547 | 133.4118899 | 7.34721E−31 | 8.22383E−29 |
| Dkkl1 | −0.607062031 | 9.511202131 | 132.8270224 | 9.86427E−31 | 1.09576E−28 |
| Ifi27l2a | −0.682729091 | 9.830872885 | 132.0563095 | 1.45435E−30 | 1.60339E−28 |
| PISD | −0.578023174 | 9.545282024 | 131.4161277 | 2.00781E−30 | 2.19705E−28 |
| Usmg5 | −0.349820825 | 9.9964598 | 130.3323663 | 3.46601E−30 | 3.7646E−28 |
| Gpr171 | 0.438173645 | 9.903960288 | 127.5732549 | 1.39168E−29 | 1.50046E−27 |
| Rgs1 | 0.397257884 | 11.60966783 | 125.5147395 | 3.92663E−29 | 4.20264E−27 |
| Fxyd5 | 0.228232386 | 10.90619274 | 125.1071312 | 4.82199E−29 | 5.12354E−27 |
| Ppp3r1 | −0.54142838 | 9.487871313 | 124.5160325 | 6.49522E−29 | 6.85176E−27 |
| Rpl37 | −0.161343968 | 11.81568565 | 124.1329903 | 7.87824E−29 | 8.25133E−27 |
| Cmtm7 | 0.454020607 | 9.769874757 | 122.7886537 | 1.55123E−28 | 1.61317E−26 |
| Prelid1 | 0.389762152 | 9.89081496 | 122.7394091 | 1.59021E−28 | 1.64206E−26 |
| Cox5a | 0.261555498 | 10.46343799 | 122.650577 | 1.66303E−28 | 1.70524E−26 |
| Cd4 | −0.379389302 | 9.985453892 | 121.4080927 | 3.11084E−28 | 3.16766E−26 |
| H3f3b | 0.195676561 | 11.62147274 | 120.5475044 | 4.80034E−28 | 4.8543E−26 |
| Sfpq | 0.426928473 | 9.766528868 | 119.9027458 | 6.64394E−28 | 6.67261E−26 |
| Rpl23a-ps3 | −0.284048391 | 10.26446957 | 118.2728016 | 1.51108E−27 | 1.50728E−25 |
| Ctsw | 0.287951157 | 10.51671124 | 118.1709245 | 1.59072E−27 | 1.576E−25 |
| Il27ra | −0.536474502 | 9.51098448 | 116.3402136 | 4.00371E−27 | 3.94003E−25 |
| Nab1 | 0.501005233 | 9.58946001 | 115.9990896 | 4.75515E−27 | 4.64832E−25 |
| Rpl39 | −0.176812196 | 11.51658061 | 115.8551826 | 5.11303E−27 | 4.96505E−25 |
| Esm1 | 0.580091419 | 9.435676786 | 115.739511 | 5.42012E−27 | 5.22864E−25 |
| Rpl31 | 0.236225624 | 10.69362592 | 113.5096718 | 1.66865E−26 | 1.59918E−24 |
| Glipr2 | 0.451381886 | 9.775168242 | 113.0218075 | 2.13415E−26 | 2.02059E−24 |
| Rps26 | 0.232520083 | 10.83772226 | 113.0201539 | 2.13593E−26 | 2.02059E−24 |
| Rps5 | 0.148067744 | 11.95622251 | 112.7529071 | 2.44413E−26 | 2.29732E−24 |
| S1pr1 | 0.588401834 | 9.457285155 | 112.1393936 | 3.33051E−26 | 3.11053E−24 |
| Sh3bgrl3 | −0.168091093 | 11.68103931 | 112.1252361 | 3.35438E−26 | 3.11299E−24 |
| Slc25a4 | 0.38380581 | 9.862505837 | 111.5888161 | 4.39661E−26 | 4.05456E−24 |
| Rps19 | 0.154571784 | 12.45475351 | 111.1783432 | 5.40801E−26 | 4.9561E−24 |
| Lgals1 | 0.264441023 | 11.89998374 | 111.0088631 | 5.89068E−26 | 5.3649E−24 |
| Psmb9 | −0.328731197 | 9.976063465 | 108.9172043 | 1.69208E−25 | 1.53154E−23 |
| Gnai2 | 0.222498644 | 10.64920018 | 108.8441011 | 1.75565E−25 | 1.57934E−23 |
| Mir142hg | 0.551042944 | 9.496107108 | 108.2667593 | 2.34932E−25 | 2.10049E−23 |
| Cd3e | −0.223197162 | 10.83627589 | 107.8485188 | 2.90126E−25 | 2.57826E−23 |
| Fus | −0.355382378 | 9.87695395 | 107.2238774 | 3.97617E−25 | 3.51221E−23 |
| Ppm1h | −0.510426159 | 9.531317964 | 106.7556798 | 5.03577E−25 | 4.42153E−23 |
| Rpl36 | −0.169365981 | 11.60496907 | 106.6065855 | 5.42925E−25 | 4.73864E−23 |
| Ppp1r15a | −0.547132695 | 9.584142239 | 106.4313775 | 5.93112E−25 | 5.14603E−23 |
| Ahrr | −0.268421399 | 9.388438844 | 104.7234475 | 1.40429E−24 | 1.21124E−22 |
| Erdr1 | 0.54023863 | 9.475463995 | 104.6521757 | 1.45573E−24 | 1.24826E−22 |
| Atp5k | −0.358970242 | 9.829102548 | 103.4309785 | 2.69626E−24 | 2.29856E−22 |
| Shisa5 | 0.218856868 | 11.19765366 | 103.3840867 | 2.76083E−24 | 2.34001E−22 |
| Eif3h | 0.242394534 | 10.41255751 | 102.9208658 | 3.48806E−24 | 2.9394E−22 |
| Mmp9 | −0.359554026 | 9.392330763 | 102.2123398 | 4.98782E−24 | 4.17923E−22 |
| Ubl3 | 0.463107639 | 9.607014043 | 102.0906527 | 5.30382E−24 | 4.41875E−22 |
| Pet100 | −0.433689861 | 9.632374387 | 101.939089 | 5.72555E−24 | 4.74315E−22 |
| Capzb | 0.223546007 | 10.5338718 | 101.9175981 | 5.788E−24 | 4.76795E−22 |
| Rpl26 | 0.158164308 | 11.42098645 | 101.7241051 | 6.3819E−24 | 5.22781E−22 |
| Reg3b | −0.500721243 | 9.406631246 | 101.3406774 | 7.74489E−24 | 6.30907E−22 |
| Ub15 | −0.252423517 | 10.30008476 | 99.79503049 | 1.69014E−23 | 1.3692E−21 |
| Ptpn22 | 0.348842201 | 10.01581807 | 98.94795597 | 2.59225E−23 | 2.08847E−21 |
| Bst2 | −0.480431543 | 9.573589557 | 98.81392257 | 2.77378E−23 | 2.22251E−21 |
| Ptp4a2 | 0.336132395 | 9.932198353 | 98.33705988 | 3.52899E−23 | 2.81226E−21 |
| Ndufa5 | −0.391689506 | 9.697497933 | 97.92740041 | 4.34006E−23 | 3.43991E−21 |
| Fam189b | −0.444063386 | 9.678923066 | 97.73586286 | 4.78085E−23 | 3.7689E−21 |
| Pim1 | 0.475298479 | 10.00737164 | 97.41181842 | 5.63089E−23 | 4.41528E−21 |
| Hilpda | 0.552369546 | 9.501052392 | 97.15607765 | 6.40723E−23 | 4.9973E−21 |
| Fth1 | −0.182222184 | 11.1646769 | 96.91852629 | 7.22396E−23 | 5.60449E−21 |
| Bcl2a1b | 0.376789448 | 10.02781406 | 96.24135593 | 1.01698E−22 | 7.84843E−21 |
| Psme2 | −0.259919999 | 10.31903574 | 96.15844171 | 1.06048E−22 | 8.14125E−21 |
| Rps3 | 0.130737293 | 11.81737892 | 95.9917874 | 1.15361E−22 | 8.76445E−21 |
| Rps16 | −0.128415044 | 12.15232601 | 95.99483852 | 1.15183E−22 | 8.76445E−21 |
| Bin2 | 0.33387622 | 9.94413916 | 95.89670834 | 1.21036E−22 | 9.14824E−21 |
| Timp2 | 0.500779729 | 9.554063201 | 95.71940824 | 1.32376E−22 | 9.95402E−21 |
| Rps4x | 0.141401747 | 11.71126116 | 95.58313032 | 1.4181E−22 | 1.0609E−20 |
| Agr2 | −0.405588336 | 9.399615257 | 95.57125335 | 1.42663E−22 | 1.06186E−20 |
| Ly6e | −0.239270346 | 10.72940372 | 95.54576674 | 1.44512E−22 | 1.07019E−20 |
| 2010107E04Rik | −0.282366294 | 10.06037516 | 95.05323724 | 1.85333E−22 | 1.3656E−20 |
| Ywhah | 0.314522758 | 10.03215277 | 94.92177622 | 1.98059E−22 | 1.45207E−20 |
| S1pr4 | 0.461593658 | 9.637339017 | 94.89959417 | 2.00291E−22 | 1.46113E−20 |
| Atf7ip | −0.497497145 | 9.522767261 | 94.00649346 | 3.1449E−22 | 2.28286E−20 |
| Hnrnpf | 0.206540192 | 10.67023919 | 93.76002102 | 3.56194E−22 | 2.57284E−20 |
| Cxcr3 | 0.411891354 | 9.824285959 | 93.20771164 | 4.70843E−22 | 3.3843E−20 |
| Rps15 | −0.157626985 | 11.27418535 | 92.7653109 | 5.88782E−22 | 4.21137E−20 |
| Rps18 | 0.12718884 | 12.37845441 | 92.51742519 | 6.67346E−22 | 4.75014E−20 |
| Map2k3 | 0.484872054 | 9.558650852 | 91.50771196 | 1.11158E−21 | 7.87397E−20 |
| Romo1 | −0.364175595 | 9.742699809 | 90.79819383 | 1.59099E−21 | 1.12157E−19 |
| Hic1 | −0.486819612 | 9.58081968 | 90.32504307 | 2.02079E−21 | 1.41774E−19 |
| Spink4 | −0.422462121 | 9.400067934 | 90.00579616 | 2.37463E−21 | 1.65806E−19 |
| Ang4 | −0.232083493 | 9.386587169 | 89.63651639 | 2.86193E−21 | 1.98884E−19 |
| Muc3 | −0.250250477 | 9.387731659 | 88.43624664 | 5.24999E−21 | 3.63116E−19 |
| 1500009L16Rik | 0.542706342 | 9.449829859 | 88.40371265 | 5.33705E−21 | 3.67405E−19 |
| Mien1 | −0.347724265 | 9.765232979 | 88.18655845 | 5.95633E−21 | 4.0812E−19 |
| Gramd3 | 0.55125369 | 9.499432993 | 88.06414473 | 6.33659E−21 | 4.32155E−19 |
| Pkm | 0.240126678 | 10.49678124 | 88.04399147 | 6.40148E−21 | 4.3456E−19 |
| Rpl27 | −0.157552345 | 11.22543808 | 87.99513948 | 6.56155E−21 | 4.43373E−19 |
| Dpm3 | −0.360538159 | 9.721395651 | 87.74227048 | 7.45637E−21 | 5.01527E−19 |
| Rhob | −0.532371904 | 9.442446398 | 87.18704808 | 9.87282E−21 | 6.61028E−19 |
| Cxcr6 | 0.260123251 | 11.11844547 | 86.10298628 | 1.70805E−20 | 1.13841E−18 |
| Sec61g | −0.221864807 | 10.42657775 | 86.01134149 | 1.78907E−20 | 1.18702E−18 |
| Rps8 | 0.157110222 | 11.43109006 | 85.75218409 | 2.03959E−20 | 1.34714E−18 |
| Cox7a2 | −0.253012989 | 10.14397168 | 85.33018222 | 2.5248E−20 | 1.66014E−18 |
| Il2rb | 0.4060724 | 9.69063229 | 85.14880794 | 2.76735E−20 | 1.8115E−18 |
| Cox14 | −0.351555119 | 9.765850837 | 84.88340428 | 3.16488E−20 | 2.06252E−18 |
| Srsf3 | 0.313008472 | 9.979900806 | 84.60915088 | 3.63579E−20 | 2.35892E−18 |
| Tagln2 | 0.299483696 | 10.35627371 | 84.45265033 | 3.93527E−20 | 2.54198E−18 |
| H2-Q6 | −0.394471662 | 9.687987566 | 83.5063727 | 6.35109E−20 | 4.08448E−18 |
| Arpc1b | 0.173890126 | 10.87245212 | 83.26504726 | 7.17572E−20 | 4.59466E−18 |
| Krt8 | −0.403281524 | 9.403467815 | 82.98133851 | 8.28317E−20 | 5.2807E−18 |
| Rps15a | −0.124996535 | 12.34731808 | 82.92709526 | 8.51362E−20 | 5.40412E−18 |
| Wbscr16 | −0.410455344 | 9.411788422 | 82.02708053 | 1.34235E−19 | 8.48399E−18 |
| Rpl7 | 0.154932946 | 11.11907579 | 81.95526321 | 1.39202E−19 | 8.76017E−18 |
| Penk | 1.313010388 | 9.664975606 | 81.20109876 | 2.03879E−19 | 1.27755E−17 |
| Rps7 | 0.129528661 | 11.72687307 | 80.86606628 | 2.41546E−19 | 1.50714E−17 |
| Hsd11b1 | 0.447395686 | 9.606894656 | 80.84119223 | 2.44606E−19 | 1.51977E−17 |
| Naca | 0.178706035 | 10.80182933 | 80.33963058 | 3.15282E−19 | 1.95062E−17 |
| Fcgbp | −0.217260104 | 9.386487028 | 80.30030325 | 3.2162E−19 | 1.98147E−17 |
| Gltscr2 | 0.258412919 | 10.25123713 | 79.51651322 | 4.78212E−19 | 2.9339E−17 |
| Socs3 | 0.641425478 | 9.534871697 | 79.32402869 | 5.27147E−19 | 3.22065E−17 |
| Rpl10 | 0.162358681 | 11.36672343 | 79.30037032 | 5.33497E−19 | 3.24592E−17 |
| Arpc2 | 0.162260977 | 10.92620521 | 79.11050195 | 5.87313E−19 | 3.55858E−17 |
| Gstp1 | −0.434068798 | 9.504080093 | 79.07810684 | 5.97023E−19 | 3.60253E−17 |
| Krt19 | −0.391532021 | 9.402773272 | 78.95610969 | 6.35052E−19 | 3.8163E−17 |
| Itm2c | −0.366659839 | 9.727481117 | 78.79268166 | 6.89821E−19 | 4.12851E−17 |
| Itga4 | 0.470754127 | 9.548703143 | 78.60469602 | 7.58688E−19 | 4.52222E−17 |
| mt-Co2 | 0.156269384 | 11.43908237 | 78.49872805 | 8.00498E−19 | 4.7521E−17 |
| Lamtor2 | −0.320200859 | 9.784237142 | 78.001988 | 1.02937E−18 | 6.08614E−17 |
| Rplp2 | −0.128947756 | 11.60786585 | 77.94171939 | 1.06126E−18 | 6.2495E−17 |
| Eif3e | 0.229380939 | 10.26281367 | 77.57368334 | 1.27862E−18 | 7.48572E−17 |
| Atp5c1 | 0.238726429 | 10.16724288 | 77.56940157 | 1.2814E−18 | 7.48572E−17 |
| Il7r | 0.426216078 | 9.82476784 | 77.38599496 | 1.40609E−18 | 8.18153E−17 |
| Acot9 | 0.433260317 | 9.502553768 | 76.63286792 | 2.05884E−18 | 1.19323E−16 |
| Epsti1 | 0.290518536 | 10.14939008 | 76.08122385 | 2.72234E−18 | 1.57156E−16 |
| Pitpna | 0.304228394 | 9.828351065 | 75.94567837 | 2.91577E−18 | 1.67662E−16 |
| Lgals4 | −0.431804311 | 9.417498631 | 75.87867365 | 3.0164E−18 | 1.72772E−16 |
| Ccl3 | 1.454717185 | 9.969073606 | 75.79213983 | 3.15153E−18 | 1.79809E−16 |
| Son | −0.321404034 | 9.820683121 | 75.4525749 | 3.7429E−18 | 2.12721E−16 |
| Nfkb1 | 0.419614213 | 9.561478136 | 75.28061286 | 4.0835E−18 | 2.31183E−16 |
| Tgoln1 | −0.393359511 | 9.57609321 | 75.24554336 | 4.15668E−18 | 2.3442E−16 |
| Atp5j2 | −0.213360835 | 10.28220098 | 75.01663083 | 4.66766E−18 | 2.62229E−16 |
| Car1 | −0.319128886 | 9.392996933 | 75.00343268 | 4.69896E−18 | 2.62981E−16 |
| Hnrnpdl | −0.372863275 | 9.619707402 | 74.56253495 | 5.87476E−18 | 3.27534E−16 |
| Pfn1 | 0.121037041 | 12.58660393 | 74.20797213 | 7.03058E−18 | 3.9049E−16 |
| Uqcrb | −0.231417337 | 10.14719696 | 73.86549321 | 8.36255E−18 | 4.62717E−16 |
| Clca1 | −0.232891953 | 9.388355562 | 73.82187536 | 8.54939E−18 | 4.71277E−16 |
| ler5 | −0.353397065 | 9.877196287 | 73.48444421 | 1.01432E−17 | 5.54964E−16 |
| Stk17b | 0.287659497 | 10.00946148 | 73.4861738 | 1.01344E−17 | 5.54964E−16 |
| Rpia | −0.369007261 | 9.412241636 | 73.08643749 | 1.24094E−17 | 6.76429E−16 |
| Syce1 | −0.185525854 | 9.385331258 | 72.9581642 | 1.32427E−17 | 7.19179E−16 |
| Tox | −0.427585214 | 9.428121663 | 72.61849444 | 1.57298E−17 | 8.47964E−16 |
| H2-T23 | −0.40377288 | 9.555965873 | 72.62324991 | 1.56919E−17 | 8.47964E−16 |
| Cd44 | −0.364888789 | 9.66492874 | 72.55033094 | 1.62826E−17 | 8.74548E−16 |
| Atp1b3 | 0.41433275 | 9.580707758 | 72.36048167 | 1.79268E−17 | 9.59346E−16 |
| Muc2 | −0.224942562 | 9.387606097 | 72.28636024 | 1.86129E−17 | 9.92442E−16 |
| Swt1 | −0.41385543 | 9.494442155 | 71.64189782 | 2.58019E−17 | 1.37077E−15 |
| Mpv17 | −0.378283479 | 9.414892521 | 71.13939089 | 3.32858E−17 | 1.76198E−15 |
| Xcl1 | 1.410763345 | 9.9045535 | 71.09150442 | 3.41035E−17 | 1.79878E−15 |
| Gimap6 | −0.242600873 | 10.32529467 | 71.01180327 | 3.55094E−17 | 1.86622E−15 |
| mt-Atp6 | 0.124721425 | 12.48272412 | 70.91305335 | 3.7332E−17 | 1.955E−15 |
| Slc3a2 | 0.341605532 | 9.804740905 | 70.51801022 | 4.56084E−17 | 2.37747E−15 |
| Pigr | −0.280051952 | 9.389838816 | 70.51302832 | 4.57237E−17 | 2.37747E−15 |
| Os9 | −0.333807082 | 9.73183663 | 70.40523305 | 4.82917E−17 | 2.50212E−15 |
| Gpr55 | −0.377093893 | 9.4164776 | 70.06607575 | 5.73508E−17 | 2.96104E−15 |
| Hist1h1e | 0.382731133 | 9.412783797 | 69.86440121 | 6.35246E−17 | 3.26829E−15 |
| Usp50 | −0.40726079 | 9.47788197 | 69.75271034 | 6.72254E−17 | 3.4466E−15 |
| Ifng | 0.586034647 | 10.08145339 | 68.9524593 | 1.00866E−16 | 5.15329E−15 |
| Chmp4b | 0.279912699 | 9.888899548 | 68.63971522 | 1.182E−16 | 6.01792E−15 |
| Marcksl1 | 0.566499192 | 9.462957421 | 68.27387403 | 1.42294E−16 | 7.21956E−15 |
| Arf5 | 0.21433798 | 10.20061958 | 68.25797953 | 1.43445E−16 | 7.2529E−15 |
| Ets1 | −0.265183278 | 10.06484846 | 68.00483487 | 1.63095E−16 | 8.21809E−15 |
| Errfi1 | −0.506623695 | 9.438930011 | 67.59798357 | 2.00472E−16 | 1.00669E−14 |
| Plp2 | 0.413929934 | 9.492940408 | 67.57578957 | 2.02742E−16 | 1.01461E−14 |
| Rcn1 | −0.40907025 | 9.519216091 | 66.03242907 | 4.43565E−16 | 2.20474E−14 |
| Rtp4 | −0.379831897 | 9.409532416 | 66.03488329 | 4.43013E−16 | 2.20474E−14 |
| 5430421N21Rik | 0.469971619 | 9.44775018 | 65.67951525 | 5.30545E−16 | 2.62817E−14 |
| Tpi1 | 0.400390794 | 9.596173139 | 65.25696016 | 6.57416E−16 | 3.24569E−14 |
| Grb2 | 0.295777902 | 9.791814485 | 65.08737903 | 7.16495E−16 | 3.52549E−14 |
| Arpc5 | 0.205320349 | 10.43833468 | 64.93908067 | 7.72497E−16 | 3.78834E−14 |
| Gm20069 | −0.42431626 | 9.468444875 | 64.77221814 | 8.40762E−16 | 4.10937E−14 |
| Gm27166 | −0.395023504 | 9.408886931 | 64.69484784 | 8.74432E−16 | 4.25973E−14 |
| Atp6v0e | −0.220993968 | 10.11729457 | 64.50744858 | 9.61681E−16 | 4.66925E−14 |
| Pkp3 | 0.260630633 | 10.14960699 | 64.12737993 | 1.16629E−15 | 5.64402E−14 |
| Gm8186 | −0.384808147 | 9.50310457 | 64.06797442 | 1.202E−15 | 5.77968E−14 |
| Rbms1 | 0.355457007 | 9.624050922 | 64.06762225 | 1.20221E−15 | 5.77968E−14 |
| Mns1 | −0.413951384 | 9.50381656 | 64.02853318 | 1.2263E−15 | 5.87623E−14 |
| Toporsos | −0.40742359 | 9.471023228 | 64.01861969 | 1.23249E−15 | 5.88664E−14 |
| Tspan31 | −0.373860991 | 9.600422269 | 63.53853721 | 1.5726E−15 | 7.4867E−14 |
| Tyrobp | −0.256002079 | 9.38856945 | 63.32068377 | 1.7565E−15 | 8.33514E−14 |
| Smim11 | −0.348900564 | 9.59541229 | 62.78883943 | 2.30099E−15 | 1.08837E−13 |
| Dusp5 | 0.43286125 | 9.605908356 | 62.67423675 | 2.43885E−15 | 1.14735E−13 |
| Gnb2l1 | 0.185704553 | 10.46584315 | 62.67223491 | 2.44133E−15 | 1.14735E−13 |
| Hspa8 | 0.143712814 | 11.71849799 | 62.55214913 | 2.59482E−15 | 1.21559E−13 |
| 3830403N18Rik | 0.406986395 | 9.402762899 | 62.5092234 | 2.652E−15 | 1.23842E−13 |
| Cd200r4 | −0.437039881 | 9.478380078 | 61.39944901 | 4.65941E−15 | 2.16892E−13 |
| Flna | 0.405123767 | 9.466784234 | 61.36779384 | 4.73493E−15 | 2.1971E−13 |
| Vasp | 0.208908637 | 10.30936964 | 61.32523678 | 4.83839E−15 | 2.23802E−13 |
| Sub1 | 0.172336396 | 10.71571666 | 61.2872353 | 4.93268E−15 | 2.27446E−13 |
| Slc16a3 | 0.361619599 | 9.420568619 | 61.03912894 | 5.59516E−15 | 2.57185E−13 |
| Phf11b | 0.377804723 | 9.570888004 | 60.72994541 | 6.54667E−15 | 2.99981E−13 |
| Ostf1 | 0.226133252 | 10.18287746 | 60.70239948 | 6.63892E−15 | 3.0326E−13 |
| Vimp | 0.357852382 | 9.630389398 | 60.39765297 | 7.75053E−15 | 3.52938E−13 |
| Lypd8 | −0.390113249 | 9.406877274 | 60.34483363 | 7.96132E−15 | 3.61415E−13 |
| Mrpl52 | −0.280626279 | 9.865813964 | 60.25390377 | 8.33773E−15 | 3.77334E−13 |
| Gbp9 | −0.384088329 | 9.438016299 | 60.13147871 | 8.87279E−15 | 4.00313E−13 |
| Gnb2 | 0.225652666 | 10.06366194 | 60.06352254 | 9.18448E−15 | 4.13104E−13 |
| Prkca | −0.382483897 | 9.504593373 | 59.99856572 | 9.49265E−15 | 4.2566E−13 |
| Nudt21 | 0.308378932 | 9.720296786 | 59.88459376 | 1.00586E−14 | 4.49661E−13 |
| 1700025G04Rik | 0.352987835 | 9.414190726 | 59.85240893 | 1.02244E−14 | 4.55686E−13 |
| Cd3c | −0.153434384 | 10.94640525 | 59.77094849 | 1.06565E−14 | 4.73503E−13 |
| Arhgdib | 0.152619993 | 11.04633827 | 59.54265492 | 1.19672E−14 | 5.30136E−13 |
| Ppp1cc | 0.238784091 | 10.02736042 | 59.42918784 | 1.26775E−14 | 5.59909E−13 |
| Eif1ax | 0.353361615 | 9.588431037 | 59.17084112 | 1.4456E−14 | 6.36542E−13 |
| Fau | −0.098888973 | 12.14505857 | 59.09913899 | 1.49925E−14 | 6.58188E−13 |
| Rhoc | −0.387685476 | 9.453376689 | 58.34747412 | 2.19679E−14 | 9.61536E−13 |
| Metap2 | 0.320908991 | 9.689184268 | 58.25059244 | 2.30767E−14 | 1.00707E−12 |
| Sumo3 | 0.363648648 | 9.56114032 | 58.19517047 | 2.37361E−14 | 1.03277E−12 |
| Gm12840 | 0.529072179 | 9.692861276 | 58.17118308 | 2.40273E−14 | 1.04234E−12 |
| Tmem258 | −0.239649837 | 9.950028733 | 57.86056954 | 2.81371E−14 | 1.21373E−12 |
| Cytip | 0.291537672 | 9.828704032 | 57.86012562 | 2.81435E−14 | 1.21373E−12 |
| Selplg | 0.207253382 | 10.43074656 | 57.58231875 | 3.24126E−14 | 1.39374E−12 |
| Napsa | 0.378797818 | 9.426630211 | 57.53254537 | 3.32433E−14 | 1.42528E−12 |
| Rps17 | 0.195072222 | 10.51281529 | 57.36114851 | 3.62702E−14 | 1.55052E−12 |
| Fas | 0.397724601 | 9.519041189 | 56.98223888 | 4.39766E−14 | 1.8745E−12 |
| Tbc1d10c | 0.239368702 | 10.02017144 | 56.89287049 | 4.60211E−14 | 1.95596E−12 |
| Rpl36-ps3 | −0.368028492 | 9.521129953 | 56.68238187 | 5.12204E−14 | 2.17065E−12 |
| Cox5b | 0.205874088 | 10.23822956 | 56.60953206 | 5.31536E−14 | 2.24608E−12 |
| Wbp1 | −0.35246475 | 9.588426001 | 56.54338706 | 5.49719E−14 | 2.31625E−12 |
| Gm8730 | 0.208874873 | 10.17481148 | 56.32847716 | 6.13208E−14 | 2.57635E−12 |
| Tmbim6 | −0.186680089 | 10.30239205 | 56.20299569 | 6.53619E−14 | 2.73829E−12 |
| Rpl28 | 0.170032447 | 10.65239961 | 55.96849437 | 7.36417E−14 | 3.07638E−12 |
| Ptpn1 | 0.303817614 | 9.726537881 | 55.73155029 | 8.30743E−14 | 3.46056E−12 |
| Dnajb4 | −0.355898832 | 9.426224573 | 55.55704902 | 9.07857E−14 | 3.77108E−12 |
| Rap1a | 0.229851445 | 10.02152566 | 55.44147997 | 9.62831E−14 | 3.98813E−12 |
| Clec12a | 0.407493649 | 9.44431809 | 55.2921659 | 1.03882E−13 | 4.29076E−12 |
| Cirbp | −0.294228645 | 9.79666609 | 55.26784248 | 1.05175E−13 | 4.32478E−12 |
| Zfp207 | 0.298332611 | 9.707086273 | 55.26559828 | 1.05295E−13 | 4.32478E−12 |
| Nkg7 | 0.161386214 | 11.93144149 | 55.10052132 | 1.14521E−13 | 4.69054E−12 |
| Ybx3 | 0.37881093 | 9.487988706 | 55.03788543 | 1.18229E−13 | 4.82894E−12 |
| AY036118 | −0.290741331 | 9.40297876 | 54.54808416 | 1.5169E−13 | 6.17844E−12 |
| 1110008F13Rik | 0.315450921 | 9.63797391 | 54.46226129 | 1.58462E−13 | 6.43636E−12 |
| Akt1 | 0.337984736 | 9.572351874 | 54.42636768 | 1.61383E−13 | 6.5369E−12 |
| Klf10 | −0.383277795 | 9.492201262 | 54.33969083 | 1.68661E−13 | 6.81286E−12 |
| Gpr183 | 0.432865552 | 9.527212243 | 54.26631414 | 1.75078E−13 | 7.05264E−12 |
| Rps27l | 0.285042685 | 9.82708366 | 54.23787371 | 1.7763E−13 | 7.13586E−12 |
| Zbtb20 | −0.398415973 | 9.422971 | 54.10646146 | 1.89916E−13 | 7.60856E−12 |
| Tpgs1 | 0.351098997 | 9.528091017 | 53.97780948 | 2.02767E−13 | 8.10128E−12 |
| Hpgds | −0.332016761 | 9.41069171 | 53.93805376 | 2.06911E−13 | 8.2444E−12 |
| Ppp3cc | −0.357588437 | 9.462008133 | 53.67190385 | 2.36927E−13 | 9.41481E−12 |
| Skap1 | −0.217904307 | 10.0596894 | 53.6434695 | 2.40381E−13 | 9.52625E−12 |
| Sepw1 | −0.202083637 | 10.28598242 | 53.38429242 | 2.74281E−13 | 1.08404E−11 |
| Dapk2 | −0.346083162 | 9.615406468 | 53.30411175 | 2.85708E−13 | 1.12617E−11 |
| Il12rb2 | −0.419238551 | 9.511930592 | 53.19792577 | 3.01577E−13 | 1.18553E−11 |
| Cd53 | 0.216440054 | 10.33331217 | 52.86673853 | 3.56964E−13 | 1.39811E−11 |
| Rps12 | 0.175598473 | 10.54841503 | 52.86346056 | 3.5756E−13 | 1.39811E−11 |
| Cers4 | −0.370534066 | 9.475950653 | 52.85080455 | 3.59871E−13 | 1.4034E−11 |

TABLE 2E
LPL vs Spleen

| GeneSymbol | logFC | logCPM | LR | PValue | FDR |
|---|---|---|---|---|---|
| Vps37b | 4.009558718 | 10.15969053 | 23880.81061 | 0 | 0 |
| Plac8 | 1.581001007 | 10.261266 | 2298.679714 | 0 | 0 |
| Arl4c | −1.096267234 | 9.613739122 | 2298.057615 | 0 | 0 |
| Gch1 | 1.451694506 | 9.324851002 | 2284.134024 | 0 | 0 |
| Hspe1 | 1.120375739 | 10.04122428 | 2264.096349 | 0 | 0 |
| Rps27 | 0.45403743 | 13.06487318 | 2254.517103 | 0 | 0 |
| Vcpkmt | 1.340902143 | 9.338076923 | 2247.925659 | 0 | 0 |
| Zfp36l2 | 1.282113838 | 9.632863878 | 2245.382999 | 0 | 0 |
| Clic1 | −0.600757562 | 10.48551744 | 2235.204657 | 0 | 0 |
| S1pr4 | −1.034174133 | 9.576670552 | 2210.175336 | 0 | 0 |
| Ehd1 | 1.34833462 | 9.340919328 | 2195.710203 | 0 | 0 |
| Vgll4 | 1.246877772 | 9.565538844 | 2194.672026 | 0 | 0 |
| Relb | 1.295849411 | 9.344579138 | 2181.915352 | 0 | 0 |
| Rpl41 | 0.311798353 | 12.67215529 | 2161.526683 | 0 | 0 |
| Chd2 | 1.272965567 | 9.362981374 | 2130.251594 | 0 | 0 |
| Psmb8 | −0.521650348 | 10.57770041 | 2126.610125 | 0 | 0 |
| Emb | 0.955845249 | 10.04817873 | 2119.007483 | 0 | 0 |
| Ninj1 | 1.323056016 | 9.256191245 | 2095.293443 | 0 | 0 |
| Birc3 | 1.298784799 | 9.307476648 | 2040.969039 | 0 | 0 |
| Anxa1 | −1.543033853 | 9.396439695 | 2030.512514 | 0 | 0 |
| Hsp90ab1 | 0.695394356 | 10.97592722 | 2021.815733 | 0 | 0 |
| Nrp1 | −1.298891806 | 9.180343991 | 1989.469145 | 0 | 0 |
| Gm26532 | 1.340633117 | 9.201977563 | 1986.395582 | 0 | 0 |
| Nr3c1 | 1.335661273 | 9.320572183 | 1975.598067 | 0 | 0 |
| Fos | 1.939072126 | 9.356513533 | 1949.123994 | 0 | 0 |
| Ptpn7 | −1.098586112 | 9.378744957 | 1946.818351 | 0 | 0 |
| Herpud1 | 1.160978832 | 9.421398845 | 1921.000688 | 0 | 0 |
| Lpxn | 0.972160773 | 9.577823699 | 1912.201507 | 0 | 0 |
| Dusp10 | 1.450167924 | 9.339187515 | 1910.235573 | 0 | 0 |
| Cebpb | 1.354746762 | 9.345842525 | 1906.273395 | 0 | 0 |
| Cfl1 | −0.480630683 | 11.2788147 | 2308.202655 | 0 | 0 |
| Snx18 | 1.251604623 | 9.230155635 | 1884.711722 | 0 | 0 |
| Cnbp | 0.723097233 | 10.09221857 | 2327.043345 | 0 | 0 |
| Smim3 | 1.463749514 | 9.239342615 | 2362.38165 | 0 | 0 |
| Serpinb9 | 1.701828092 | 9.633156639 | 2778.027215 | 0 | 0 |
| Nmrk1 | 1.703580609 | 9.325880651 | 2775.614 | 0 | 0 |
| Lmna | 1.988196567 | 9.500793605 | 2775.326852 | 0 | 0 |
| Kcnn4 | −1.280719622 | 9.426740809 | 2763.826967 | 0 | 0 |
| Cnn2 | −0.796651795 | 10.07688931 | 2733.152098 | 0 | 0 |
| Ets1 | 0.865935776 | 10.06873785 | 2730.781656 | 0 | 0 |
| Ifnar1 | 1.394023698 | 9.39348068 | 2702.666246 | 0 | 0 |
| Nfkb2 | 1.416451857 | 9.378018127 | 2696.575281 | 0 | 0 |
| Rpl38 | 0.479291508 | 11.50231551 | 2689.835505 | 0 | 0 |
| Kdm2b | 1.49910175 | 9.314276976 | 2668.313076 | 0 | 0 |
| Hist1h1c | 1.899413675 | 9.376902339 | 2666.207296 | 0 | 0 |
| Tnfrsf1b | 1.486664627 | 9.318747024 | 2637.622067 | 0 | 0 |
| Fosb | 1.843370572 | 9.275443905 | 2631.524006 | 0 | 0 |
| Pfn1 | −0.471336396 | 12.35410724 | 2630.65958 | 0 | 0 |
| Ly6c2 | −2.353611076 | 9.526958136 | 2598.695467 | 0 | 0 |
| Tnfsf11 | 1.874016005 | 9.378727031 | 2597.403773 | 0 | 0 |
| Rac2 | −0.470957461 | 11.26305817 | 2586.49052 | 0 | 0 |
| Per1 | 1.427684134 | 9.218646505 | 2584.621529 | 0 | 0 |
| Rrad | 1.697678281 | 9.38211759 | 2577.710629 | 0 | 0 |
| Ddit4 | −1.378670763 | 9.499428949 | 2548.030456 | 0 | 0 |
| Nrip1 | 1.534927003 | 9.422635525 | 2531.038897 | 0 | 0 |
| Dennd4a | 1.439765985 | 9.24072726 | 2516.239312 | 0 | 0 |
| Map3k8 | 1.567239393 | 9.298416412 | 2506.429161 | 0 | 0 |
| Maff | 1.476351404 | 9.20355435 | 2486.721122 | 0 | 0 |
| Gpr183 | 1.345546371 | 9.514196163 | 2445.661726 | 0 | 0 |
| Anxa6 | −0.826956439 | 9.887832738 | 2436.02059 | 0 | 0 |
| Litaf | 1.609729506 | 9.308467153 | 2431.541523 | 0 | 0 |
| Tbc1d10c | −0.950810827 | 9.643337868 | 2373.820075 | 0 | 0 |
| Rpl37a | 0.39133888 | 12.46681026 | 2368.041434 | 0 | 0 |
| Rbm39 | 0.607400682 | 10.52053927 | 2327.915783 | 0 | 0 |
| Actr3 | −0.664337821 | 10.4077094 | 2779.972955 | 0 | 0 |
| Plcxd2 | 1.339741239 | 9.251069352 | 1883.281793 | 0 | 0 |
| Tspo | −0.647613294 | 10.04645974 | 1882.222917 | 0 | 0 |
| Cdc42 | −0.463632709 | 10.51588855 | 1603.493133 | 0 | 0 |
| Selk | 0.625079555 | 9.913225022 | 1602.885033 | 0 | 0 |
| Zfp3611 | 1.170501061 | 9.40624196 | 1597.645418 | 0 | 0 |
| Nr4a3 | 1.016389034 | 9.172429909 | 1596.136019 | 0 | 0 |
| Cxcl10 | 1.598883919 | 9.240851428 | 1591.202272 | 0 | 0 |
| Zyx | −1.045672086 | 9.373220658 | 1590.948124 | 0 | 0 |
| Lin54 | 1.190845071 | 9.222484632 | 1579.909581 | 0 | 0 |
| Rpl37 | 0.321884655 | 11.9503646 | 1579.248798 | 0 | 0 |
| mt-Cytb | −0.421897009 | 11.23550207 | 1579.172468 | 0 | 0 |
| Hnrnpf | −0.455123386 | 10.52390819 | 1578.292187 | 0 | 0 |
| Peli1 | 0.916803098 | 9.61652175 | 1577.029497 | 0 | 0 |
| Malat1 | 0.546709942 | 14.46716245 | 1575.560381 | 0 | 0 |
| Insig1 | 1.23314691 | 9.265929909 | 1572.809776 | 0 | 0 |
| Ppp1r16b | 1.143580107 | 9.365739316 | 1572.696243 | 0 | 0 |
| Reep5 | −0.746733789 | 9.737965346 | 1566.865393 | 0 | 0 |
| Gpx4 | 0.724736901 | 9.927359165 | 1560.006316 | 0 | 0 |
| Ifng | 1.694424607 | 9.500982203 | 1559.868513 | 0 | 0 |
| Fkbp1a | −0.649924112 | 9.83535715 | 1555.689567 | 0 | 0 |
| Stx11 | 1.159910256 | 9.327908379 | 1555.317141 | 0 | 0 |
| Socs3 | 1.280365319 | 9.361846802 | 1552.722296 | 0 | 0 |
| Phlda1 | 1.392376646 | 9.315470837 | 1528.438748 | 0 | 0 |
| Mfsd6 | 1.164224359 | 9.264014917 | 1526.311577 | 0 | 0 |
| Gzmb | 1.732500572 | 10.06757702 | 1511.117786 | 0 | 0 |
| Hbp1 | 1.068823826 | 9.308365307 | 1505.133032 | 0 | 0 |
| Traf4 | 1.168895052 | 9.255444042 | 1498.895049 | 0 | 0 |
| Ddit3 | 1.179486367 | 9.269696228 | 1491.285564 | 0 | 0 |
| Dtx4 | 1.034303937 | 9.178017295 | 1490.811143 | 0 | 0 |
| Sik1 | 1.038369744 | 9.179583562 | 1489.469987 | 0 | 0 |
| AB124611 | −1.056831806 | 9.18576542 | 1487.66957 | 0 | 0 |
| Cdc42se1 | −0.903345939 | 9.439589721 | 1606.39814 | 0 | 0 |
| Gabpb1 | 1.275991664 | 9.34485467 | 1882.835108 | 0 | 0 |
| Kpna1 | 1.188926205 | 9.266532319 | 1609.29646 | 0 | 0 |
| Ldlrad4 | 1.213360812 | 9.205367734 | 1622.44325 | 0 | 0 |
| Sp100 | −0.864441892 | 9.662295373 | 1868.043867 | 0 | 0 |
| Gpr171 | 1.021072703 | 9.57636816 | 1834.625452 | 0 | 0 |
| Atf3 | 1.325297273 | 9.196373801 | 1802.980884 | 0 | 0 |
| Tnfaip8l2 | −1.05719081 | 9.395004626 | 1800.880544 | 0 | 0 |
| Spn | −0.928132208 | 9.500542247 | 1791.461043 | 0 | 0 |
| Plscr1 | 1.309849059 | 9.383402199 | 1789.387598 | 0 | 0 |
| Aebp2 | 1.116047127 | 9.343970926 | 1785.470204 | 0 | 0 |
| Dnajb9 | 1.24033724 | 9.281272707 | 1779.954261 | 0 | 0 |
| Arl6ip5 | −0.682541103 | 9.890325667 | 1770.083233 | 0 | 0 |
| Tob2 | 1.323296597 | 9.313589329 | 1750.65385 | 0 | 0 |
| Ppp1ca | −0.517869863 | 10.43097858 | 1746.20318 | 0 | 0 |
| Ifi27l2a | −1.251027487 | 10.08297152 | 1740.339055 | 0 | 0 |
| Crip1 | −0.764501171 | 11.97479307 | 1738.170641 | 0 | 0 |
| Wdr1 | −0.778630406 | 9.661982528 | 1729.765806 | 0 | 0 |
| Stat3 | 1.077543403 | 9.486265548 | 1729.317606 | 0 | 0 |
| Nabp1 | 1.103465745 | 9.487243674 | 1721.614948 | 0 | 0 |
| Arpc3 | 0.554163297 | 10.38658938 | 1713.609041 | 0 | 0 |
| Hk2 | 1.233253218 | 9.197875054 | 1701.077294 | 0 | 0 |
| Prkcq | −1.046433802 | 9.3213095 | 1691.976912 | 0 | 0 |
| Areg | 1.913703104 | 9.255472455 | 1691.701767 | 0 | 0 |
| Rgcc | 1.578501766 | 9.406425986 | 1661.879727 | 0 | 0 |
| Bcl2l11 | 1.250982211 | 9.230833464 | 1657.235805 | 0 | 0 |
| Uhrf2 | 1.180900948 | 9.419077555 | 1649.885224 | 0 | 0 |
| Prdx1 | −0.634280751 | 10.07947019 | 1648.801939 | 0 | 0 |
| Jun | 1.867730862 | 9.427464308 | 1639.113455 | 0 | 0 |
| Ywhaq | −0.743677921 | 9.740986041 | 1631.460167 | 0 | 0 |
| Nfkbie | 1.19011788 | 9.287419751 | 1627.853905 | 0 | 0 |
| Fut7 | −1.062699209 | 9.158108524 | 1627.249297 | 0 | 0 |
| Rpl36 | 0.367174335 | 11.74359785 | 1627.246697 | 0 | 0 |
| Txndc5 | −1.090326933 | 9.243654724 | 1617.22599 | 0 | 0 |
| Ppia | −0.511777146 | 11.74358668 | 2794.793642 | 0 | 0 |
| Sema4a | −1.424308061 | 9.241797243 | 2470.2212 | 0 | 0 |
| S100a6 | −1.045197044 | 11.39764863 | 2830.471179 | 0 | 0 |
| Itgb1 | −2.897673381 | 9.764726657 | 7266.956354 | 0 | 0 |
| Nr4a1 | 2.692070423 | 9.419456526 | 7059.962503 | 0 | 0 |
| Btg2 | 1.671637356 | 10.22813028 | 7057.938285 | 0 | 0 |
| Ifrd1 | 2.113629694 | 9.751098304 | 6942.518787 | 0 | 0 |
| Slc3a2 | 1.818065901 | 9.824654011 | 6939.012246 | 0 | 0 |
| Rnf157 | 1.564061754 | 9.236925083 | 2811.277242 | 0 | 0 |
| Cd48 | −1.552731996 | 10.08313072 | 6733.969626 | 0 | 0 |
| Orai1 | 2.031182755 | 9.642689825 | 6709.374791 | 0 | 0 |
| Ms4a6b | −1.10429405 | 10.51708752 | 6525.231353 | 0 | 0 |
| Mxd1 | 2.454007515 | 9.403127833 | 6451.949777 | 0 | 0 |
| Gpr132 | 1.862994857 | 9.638154261 | 6304.793777 | 0 | 0 |
| Prdx6 | 1.568918578 | 9.939310118 | 6301.185371 | 0 | 0 |
| Nfkbiz | 2.356142607 | 9.431478134 | 6262.978333 | 0 | 0 |
| Fth1 | 0.904024109 | 11.31936277 | 6242.555727 | 0 | 0 |
| Bcl2a1d | 2.042665993 | 9.693840798 | 6226.697766 | 0 | 0 |
| Ddx5 | 0.952948791 | 10.94169958 | 6176.842997 | 0 | 0 |
| Neurl3 | 2.291995793 | 9.422883382 | 6083.82123 | 0 | 0 |
| Pde4b | 2.141569839 | 9.459117556 | 6048.258702 | 0 | 0 |
| Ramp3 | 2.930687962 | 9.642864931 | 5988.463883 | 0 | 0 |
| Cd52 | −0.796245466 | 12.13359984 | 5974.617413 | 0 | 0 |
| Kdm6b | 2.638646687 | 9.504202492 | 5850.261738 | 0 | 0 |
| Coro1a | −0.838091837 | 11.0659365 | 5816.078249 | 0 | 0 |
| Fosl2 | 2.335832539 | 9.352839289 | 5695.725353 | 0 | 0 |
| Got1 | 2.334162607 | 9.599631296 | 5680.076036 | 0 | 0 |
| Gramd3 | 1.916759134 | 9.918543027 | 5620.679858 | 0 | 0 |
| Zc3h12a | 2.176380795 | 9.362848531 | 5610.099047 | 0 | 0 |
| Klf2 | −1.767005406 | 10.48569203 | 5373.578517 | 0 | 0 |
| Gadd45b | 2.426306327 | 9.500826601 | 5239.796282 | 0 | 0 |
| Hif1a | 1.780128237 | 9.565953875 | 5140.654989 | 0 | 0 |
| Nfkbia | 2.074463071 | 10.45274542 | 7539.224662 | 0 | 0 |
| Emp3 | −1.213102789 | 10.15411072 | 5128.633776 | 0 | 0 |
| Traf1 | 1.897847312 | 9.834035476 | 7663.691793 | 0 | 0 |
| Akap13 | 1.663872351 | 10.02648752 | 8049.393054 | 0 | 0 |
| Junb | 2.921231675 | 11.34612064 | 21369.42787 | 0 | 0 |
| Btg1 | 2.116029741 | 11.76687431 | 18450.45163 | 0 | 0 |
| Tnfaip3 | 3.546622186 | 9.77629499 | 17313.81847 | 0 | 0 |
| Ubald2 | 2.898183798 | 10.20759585 | 16609.50842 | 0 | 0 |
| Rgs1 | 3.741819446 | 10.58284028 | 16488.72974 | 0 | 0 |
| H3f3b | 1.419705723 | 11.96347813 | 16463.45133 | 0 | 0 |
| Bhlhe40 | 2.475029045 | 10.31956761 | 15891.72535 | 0 | 0 |
| Jund | 2.107406468 | 11.36637401 | 15261.14463 | 0 | 0 |
| Ctla4 | 3.553585624 | 9.803253865 | 14522.85331 | 0 | 0 |
| Pnrc1 | 2.15434723 | 10.22062858 | 13387.98624 | 0 | 0 |
| Pim1 | 2.650738825 | 10.39640396 | 12688.13388 | 0 | 0 |
| Odc1 | 3.273282194 | 10.05458095 | 12438.55364 | 0 | 0 |
| Ms4a4b | −1.388879734 | 11.2982785 | 12023.83633 | 0 | 0 |
| Hspa5 | 1.762426402 | 10.61032756 | 10372.84498 | 0 | 0 |
| Dusp5 | 2.558389281 | 9.706009226 | 10293.91778 | 0 | 0 |
| Crem | 3.182372679 | 9.742097098 | 10249.83742 | 0 | 0 |
| Tnfrsf4 | 2.842699768 | 10.21808516 | 9824.168095 | 0 | 0 |
| Ifngr1 | 1.731994699 | 10.73718794 | 9664.17174 | 0 | 0 |
| Eif1 | 0.89426742 | 11.59142126 | 9507.112454 | 0 | 0 |
| Furin | 3.228695217 | 9.993985472 | 9448.5407 | 0 | 0 |
| Ubb | 1.103098222 | 12.5650094 | 9356.998296 | 0 | 0 |
| Dusp1 | 3.172241508 | 9.640554712 | 9051.339287 | 0 | 0 |
| Bcl2a1b | 2.039441995 | 10.06905308 | 8924.378246 | 0 | 0 |
| Rgs2 | 3.216003123 | 9.735808693 | 8865.377702 | 0 | 0 |
| Tgif1 | 2.669467418 | 9.488421786 | 8609.872205 | 0 | 0 |
| Icos | 2.257604109 | 9.939093266 | 8555.625143 | 0 | 0 |
| Gna13 | 2.210200992 | 9.714523893 | 8465.331307 | 0 | 0 |
| Il21r | 2.276530222 | 9.883875602 | 8187.01809 | 0 | 0 |
| Prr7 | 2.588699784 | 9.57070472 | 8146.897164 | 0 | 0 |
| Srgn | 1.128130336 | 11.07156455 | 8022.942843 | 0 | 0 |
| Nr4a2 | 2.380718393 | 9.345009261 | 5127.819344 | 0 | 0 |
| Dgat1 | 2.502339096 | 9.536306398 | 6766.351571 | 0 | 0 |
| Lilr4b | 2.068066286 | 9.554402213 | 5105.653595 | 0 | 0 |
| Pglyrp1 | −1.578157174 | 9.494896552 | 3736.717229 | 0 | 0 |
| Clk1 | 1.412333559 | 9.582346831 | 3707.927922 | 0 | 0 |
| Hsp90aa1 | 1.398095596 | 10.45163337 | 3583.071262 | 0 | 0 |
| S100a10 | −0.838341879 | 11.23216634 | 3576.86891 | 0 | 0 |
| Srsf5 | 1.06692542 | 9.897475047 | 3538.850894 | 0 | 0 |
| AI467606 | −1.477840682 | 9.366543594 | 3529.615644 | 0 | 0 |
| D16Ertd472e | 1.538790532 | 9.456346393 | 3480.361308 | 0 | 0 |
| Pycard | −1.186370114 | 9.771692598 | 3477.954478 | 0 | 0 |
| Ptpn22 | 1.180249407 | 9.888180464 | 3456.544416 | 0 | 0 |
| Il4ra | 1.73212666 | 9.347559129 | 3449.594642 | 0 | 0 |
| Hsd11b1 | −1.455842293 | 9.399808528 | 3413.567354 | 0 | 0 |
| Cirbp | 1.295170619 | 9.642760985 | 3349.752172 | 0 | 0 |
| Dnaja1 | 1.152714388 | 10.49300516 | 3345.386455 | 0 | 0 |
| Myl6 | −0.594146002 | 11.12573097 | 3316.559472 | 0 | 0 |
| Fasl | 1.646552093 | 9.427586977 | 3258.871927 | 0 | 0 |
| Cd28 | 1.075016619 | 10.15896389 | 3221.672715 | 0 | 0 |
| Csrnp1 | 1.751004019 | 9.322536386 | 3211.316895 | 0 | 0 |
| Sqstm1 | 1.424220111 | 9.492387286 | 3195.177459 | 0 | 0 |
| Klf6 | 1.575786258 | 9.681592634 | 3153.720756 | 0 | 0 |
| Ptprcap | −0.830524912 | 10.24764013 | 3140.577969 | 0 | 0 |
| Dnajb6 | 1.025227273 | 9.838018226 | 3095.166709 | 0 | 0 |
| Coq10b | 1.529034118 | 9.363466488 | 3063.979793 | 0 | 0 |
| Rel | 1.688619703 | 9.305031672 | 3052.495801 | 0 | 0 |
| Itgb2 | −1.323533083 | 9.794213175 | 5122.905985 | 0 | 0 |
| Rnf125 | 1.43084709 | 9.410691476 | 3015.190192 | 0 | 0 |
| Atp1b3 | −1.188356367 | 9.541177167 | 2959.565466 | 0 | 0 |
| Crbn | 1.35989983 | 9.537037527 | 2862.044372 | 0 | 0 |
| Lilrb4a | 1.62136421 | 9.469548765 | 2849.108644 | 0 | 0 |
| Tnfsf8 | 2.003699437 | 9.748893292 | 2841.22511 | 0 | 0 |
| Fam110a | 1.855968483 | 9.339616307 | 3742.547868 | 0 | 0 |
| Lsp1 | −0.85697683 | 10.56861487 | 3749.810676 | 0 | 0 |
| Ccnd3 | −1.115585967 | 9.67648159 | 3025.973562 | 0 | 0 |
| Stk17b | 1.121601456 | 9.887717235 | 3796.865615 | 0 | 0 |
| Hilpda | 2.295082881 | 9.459740509 | 5096.687516 | 0 | 0 |
| P2ry10 | 2.00384552 | 9.406978436 | 5064.082883 | 0 | 0 |
| Skil | 2.104468774 | 9.381816818 | 5012.624057 | 0 | 0 |
| Itgb7 | −1.084543942 | 10.22214203 | 4961.314584 | 0 | 0 |
| Ubc | 1.107062199 | 10.77239943 | 4775.13384 | 0 | 0 |
| Hspa8 | 0.861483141 | 12.05339949 | 4723.217104 | 0 | 0 |
| Arf4 | 1.361714648 | 9.759651944 | 4573.762943 | 0 | 0 |
| S100a4 | −1.822225081 | 10.3993079 | 4512.496598 | 0 | 0 |
| Lgals1 | −1.14532414 | 12.13039325 | 4400.084682 | 0 | 0 |
| Samsn1 | 1.812848974 | 9.577321817 | 4380.19289 | 0 | 0 |
| Rora | 1.625105758 | 9.750458338 | 4279.038537 | 0 | 0 |
| Cd69 | 1.956584843 | 9.648469291 | 4244.509669 | 0 | 0 |
| Itk | 1.558211507 | 9.611291599 | 4240.243249 | 0 | 0 |
| Arid5a | 1.670309896 | 9.477417365 | 4224.262832 | 0 | 0 |
| Dnajb1 | 2.4916257 | 9.971238873 | 4317.921832 | 0 | 0 |
| Arhgap31 | 1.920161613 | 9.463440841 | 4211.687512 | 0 | 0 |
| Tnfrsf18 | 1.32855039 | 9.848184871 | 3814.199865 | 0 | 0 |
| Gm12840 | 2.186828689 | 9.862794257 | 3868.008163 | 0 | 0 |
| Ctsd | −1.262237547 | 9.86539271 | 4217.656935 | 0 | 0 |
| Sh2d2a | 1.535581984 | 9.642030852 | 4029.081971 | 0 | 0 |
| Cytip | 1.135941313 | 9.972903526 | 4032.840636 | 0 | 0 |
| Hspa1a | 3.929295111 | 10.49028171 | 3753.849294 | 0 | 0 |
| Ptp4a1 | 1.541286869 | 9.515613922 | 4057.508994 | 0 | 0 |
| Cdkn1a | 2.230173637 | 9.480758236 | 3950.6614 | 0 | 0 |
| Arhgdib | −0.675912229 | 10.95838921 | 4102.421066 | 0 | 0 |
| Tspan32 | −1.853896914 | 9.311138568 | 4112.407261 | 0 | 0 |
| Bin2 | −1.121802636 | 9.796752534 | 4061.613939 | 0 | 0 |
| Glipr2 | −1.355596511 | 9.677643142 | 4113.247068 | 0 | 0 |
| Rasgrp2 | −1.490656382 | 9.547266701 | 4194.458667 | 0 | 0 |
| Ppp1r15a | 1.921918974 | 9.417222937 | 4206.958192 | 0 | 0 |
| Rps27l | −0.790585793 | 9.634266967 | 1451.086527 | 0 | 0 |
| Lztfl1 | 1.239473856 | 9.305427356 | 1450.149784 | 0 | 0 |
| Tmem123 | 0.845950877 | 9.530098905 | 1448.989968 | 0 | 0 |
| Actn2 | 1.250413478 | 9.425542778 | 1448.693327 | 0 | 0 |
| Ndufa4 | −0.671796207 | 9.843210891 | 1446.791341 | 0 | 0 |
| Vim | −0.707157046 | 10.6775884 | 1442.832741 | 0 | 0 |
| Arpc1b | −0.403868405 | 10.67217992 | 1440.52746 | 0 | 0 |
| Phf11b | −0.946380058 | 9.358275331 | 1438.351162 | 0 | 0 |
| Capn2 | −0.91793996 | 9.369740513 | 1428.333582 | 0 | 0 |
| Mxi1 | 1.087678308 | 9.216625774 | 1426.267933 | 0 | 0 |
| Tgif2 | 1.040060076 | 9.18888498 | 1415.219631 | 0 | 6.605E−308 |
| Apobec3 | −0.782739383 | 9.585116167 | 1403.461263 | 3.7178E−307 | 2.3626E−305 |
| Tnfrsf9 | 1.369904528 | 9.278619133 | 1396.683257 | 1.1045E−305 | 6.9933E−304 |
| Esyt1 | −0.897252948 | 9.384944625 | 1382.905158 | 1.0894E−302 | 6.8727E−301 |
| Flna | −0.978570065 | 9.307490367 | 1380.264205 | 4.084E−302 | 2.5671E−300 |
| Usmg5 | 0.63064033 | 9.923002096 | 1378.078435 | 1.2192E−301 | 7.6359E−300 |
| Adora2a | 1.059391205 | 9.316041201 | 1370.747214 | 4.7772E−300 | 2.9814E−298 |
| D8Ertd738e | 0.617633538 | 9.895276459 | 1370.032005 | 6.8326E−300 | 4.2489E−298 |
| Id3 | −1.333328527 | 9.200499915 | 1367.64552 | 2.2552E−299 | 1.3974E−297 |
| Prdx4 | −1.059670059 | 9.193532854 | 1363.160708 | 2.127E−298 | 1.3133E−296 |
| Sdf4 | 0.70306217 | 9.785099467 | 1362.622111 | 2.7849E−298 | 1.7134E−296 |
| Nfil3 | 1.075323099 | 9.192168652 | 1362.527538 | 2.9198E−298 | 1.7901E−296 |
| Il2rg | 0.652629336 | 9.986962428 | 1360.726772 | 7.1891E−298 | 4.3921E−296 |
| H2-Q4 | 0.817421022 | 9.616635172 | 1357.777577 | 3.1445E−297 | 1.9144E−295 |
| Gimap6 | 0.618227968 | 10.30516051 | 1355.495711 | 9.8496E−297 | 5.9756E−295 |
| Zeb1 | 1.106302599 | 9.265995084 | 1354.501578 | 1.6198E−296 | 9.7928E−295 |
| Avl9 | 1.038144975 | 9.286737515 | 1345.10992 | 1.7796E−294 | 1.0722E−292 |
| Rps27rt | 0.555934358 | 11.01209013 | 1344.386415 | 2.556E−294 | 1.5346E−292 |
| Btg3 | 1.056866822 | 9.23085599 | 1337.533638 | 7.8836E−293 | 4.7172E−291 |
| Rsrp1 | −0.606429022 | 10.12693264 | 1333.093968 | 7.2695E−292 | 4.3348E−290 |
| Gngt2 | 1.116293195 | 9.262770571 | 1332.833959 | 8.2796E−292 | 4.9203E−290 |
| Gm20186 | 1.128143873 | 9.208968634 | 1331.733776 | 1.4358E−291 | 8.5034E−290 |
| Lck | −0.448015594 | 10.43154115 | 1327.416216 | 1.2455E−290 | 7.3514E−289 |
| Zcchc11 | 1.033365137 | 9.25900753 | 1326.729463 | 1.7562E−290 | 1.0331E−288 |
| Dtx1 | −1.112944741 | 9.293744389 | 1324.589255 | 5.1247E−290 | 3.0044E−288 |
| Polg2 | 1.008383697 | 9.189277582 | 1324.203906 | 6.2146E−290 | 3.6311E−288 |
| Pde4d | 0.978758479 | 9.184653426 | 1318.71138 | 9.7051E−289 | 5.6517E−287 |
| Il22 | 2.340701289 | 9.319200911 | 1307.314053 | 2.9092E−286 | 1.6885E−284 |
| Ms4a4c | −1.131637582 | 9.194976637 | 1297.197315 | 4.595E−284 | 2.6581E−282 |
| Map3k14 | 1.068225388 | 9.243899938 | 1290.032314 | 1.6571E−282 | 9.5541E−281 |
| Dusp4 | 0.943758908 | 9.167405616 | 1289.59139 | 2.0662E−282 | 1.1873E−280 |
| Hexb | −0.955449076 | 9.253917287 | 1284.064918 | 3.2821E−281 | 1.8799E−279 |
| Calm1 | −0.398344969 | 10.95709394 | 1280.097217 | 2.39E−280 | 1.3644E−278 |
| S100a13 | −0.556791844 | 9.994311271 | 1273.779414 | 5.6411E−279 | 3.2099E−277 |
| Lfng | −0.911433167 | 9.448019615 | 1270.087278 | 3.5788E−278 | 2.0298E−276 |
| Abcb1a | 1.030096997 | 9.210772627 | 1262.459651 | 1.6269E−276 | 9.1972E−275 |
| Cyba | −0.443659225 | 10.54811046 | 1258.623632 | 1.1092E−275 | 6.2501E−274 |
| Tmsb4x | −0.267366545 | 14.35989227 | 1257.793098 | 1.6807E−275 | 9.4401E−274 |
| Ccr5 | 1.14137339 | 9.386994832 | 1254.698733 | 7.906E−275 | 4.4263E−273 |
| Dynll1 | −0.533721052 | 10.16004138 | 1245.450142 | 8.0885E−273 | 4.514E−271 |
| Cdc25b | −1.011124608 | 9.240089381 | 1241.171393 | 6.8823E−272 | 3.8286E−270 |
| Itpkb | 0.954598204 | 9.393345749 | 1231.44857 | 8.9273E−270 | 4.9504E−268 |
| Ifi47 | −1.019302639 | 9.324191671 | 1226.120429 | 1.2843E−268 | 7.0989E−267 |
| Pim3 | 1.120079893 | 9.232710072 | 1223.737702 | 4.2313E−268 | 2.3315E−266 |
| Mir142hg | 1.002778292 | 9.203588598 | 1221.931528 | 1.0447E−267 | 5.7384E−266 |
| Slc38a2 | 0.980080989 | 9.356984714 | 1217.969539 | 7.5865E−267 | 4.154E−265 |
| Add3 | −0.936691018 | 9.260409169 | 1206.100053 | 2.8813E−264 | 1.5727E−262 |
| H2-Q6 | 0.761009186 | 9.610491146 | 1203.68587 | 9.644E−264 | 5.2475E−262 |
| Asb2 | 1.117112448 | 9.36183005 | 1199.915336 | 6.3635E−263 | 3.4518E−261 |
| Sesn3 | 0.935543404 | 9.346703299 | 1196.855271 | 2.9426E−262 | 1.5912E−260 |
| Fus | 0.646782373 | 9.766633699 | 1195.296437 | 6.4197E−262 | 3.4607E−260 |
| Myo1f | −0.873721079 | 9.342854295 | 1195.091822 | 7.1119E−262 | 3.822E−260 |
| Hsph1 | 1.166689856 | 9.349269322 | 1192.706446 | 2.3464E−261 | 1.2571E−259 |
| Ptpn6 | −0.882603255 | 9.388975554 | 1187.971213 | 2.509E−260 | 1.3401E−258 |
| Def6 | −0.806896845 | 9.38910456 | 1185.235824 | 9.8624E−260 | 5.2515E−258 |
| Sub1 | 0.452332616 | 11.00148656 | 1179.659467 | 1.6066E−258 | 8.5285E−257 |
| Abcb1b | 1.031343273 | 9.207220814 | 1177.281825 | 5.28E−258 | 2.7944E−256 |
| 4930523C07Rik | 0.720136823 | 9.698992642 | 1167.683789 | 6.4357E−256 | 3.3957E−254 |
| Traf3ip3 | −0.853000826 | 9.34798616 | 1163.769696 | 4.5631E−255 | 2.4004E−253 |
| Ube2b | 0.610256432 | 9.791411125 | 1160.573155 | 2.2593E−254 | 1.1849E−252 |
| Sytl3 | 0.90845429 | 9.39100109 | 1157.949128 | 8.3997E−254 | 4.392E−252 |
| Smad7 | 0.993817761 | 9.276173853 | 1155.62533 | 2.6872E−253 | 1.4009E−251 |
| Casp4 | 0.970497764 | 9.304035945 | 1150.905397 | 2.8518E−252 | 1.4822E−250 |
| Cxcr3 | −0.782625551 | 9.636541469 | 1140.943197 | 4.1712E−250 | 2.1616E−248 |
| Zc3hav1 | 0.783012423 | 9.545786673 | 1139.080842 | 1.0593E−249 | 5.4732E−248 |
| Prkar1a | 0.51534633 | 9.989784924 | 1129.99109 | 1.0013E−247 | 5.1583E−246 |
| Stat4 | 0.87162553 | 9.393938041 | 1124.641897 | 1.456E−246 | 7.4785E−245 |
| Pabpc1 | 0.354811986 | 10.74945661 | 1118.678903 | 2.8785E−245 | 1.4741E−243 |
| Tpst2 | −0.683138851 | 9.562387953 | 1118.595548 | 3.0011E−245 | 1.5324E−243 |
| Camk2n1 | 1.044877893 | 9.246195314 | 1099.512516 | 4.2154E−241 | 2.1461E−239 |
| Rcsd1 | −0.841278225 | 9.314558222 | 1097.472831 | 1.1699E−240 | 5.9389E−239 |
| Irf4 | 0.93980886 | 9.17631645 | 1097.239553 | 1.3148E−240 | 6.6549E−239 |
| Fam65b | −0.901719998 | 9.278382726 | 1091.496759 | 2.3282E−239 | 1.175E−237 |
| Lbr | −0.742593678 | 9.489271039 | 1091.478886 | 2.3491E−239 | 1.1822E−237 |
| Apbb1ip | −0.734076752 | 9.476988831 | 1091.350028 | 2.5056E−239 | 1.2573E−237 |
| Ctla2a | 0.918347724 | 10.12270773 | 1087.781562 | 1.4945E−238 | 7.4778E−237 |
| Ndfip1 | 0.723456574 | 9.542168744 | 1087.435345 | 1.7773E−238 | 8.867E−237 |
| Ubl5 | 0.460259851 | 10.14988664 | 1085.990773 | 3.6621E−238 | 1.8218E−236 |
| Fam46a | 1.216427371 | 9.373607718 | 1084.141952 | 9.2377E−238 | 4.5825E−236 |
| Kcnab2 | −0.822790253 | 9.316762961 | 1080.193997 | 6.6626E−237 | 3.2957E−235 |
| Ahr | 1.055054007 | 9.234738845 | 1079.788424 | 8.1619E−237 | 4.0259E−235 |
| Hbb-bs | −0.875475832 | 9.153071488 | 1078.386874 | 1.646E−236 | 8.0958E−235 |
| Osgin1 | 0.942209138 | 9.166127069 | 1076.502703 | 4.2261E−236 | 2.0728E−234 |
| Il12rb2 | 1.030381194 | 9.249916639 | 1075.546924 | 6.8183E−236 | 3.3348E−234 |
| Fau | 0.234758491 | 12.21265562 | 1071.940035 | 4.146E−235 | 2.0221E−233 |
| Mier1 | 0.825393149 | 9.428014107 | 1070.148889 | 1.0161E−234 | 4.942E−233 |
| mt-Co3 | −0.275253772 | 12.22313569 | 1069.075336 | 1.7389E−234 | 8.4338E−233 |
| Rasa3 | −0.903689186 | 9.255738017 | 1058.61195 | 3.2696E−232 | 1.5814E−230 |
| E2f2 | −0.8392467 | 9.159524644 | 1053.99296 | 3.2995E−231 | 1.5914E−229 |
| Ptp4a2 | 0.574398647 | 9.844619398 | 1050.023682 | 2.4054E−230 | 1.157E−228 |
| Rpl27 | 0.281823678 | 11.36103815 | 1048.388203 | 5.4533E−230 | 2.6158E−228 |
| Pomp | −0.550362574 | 9.774637428 | 1047.45567 | 8.6966E−230 | 4.16E−228 |
| Ube2j1 | 0.830125377 | 9.345358827 | 1046.789853 | 1.2136E−229 | 5.7893E−228 |
| Dbi | −0.752178664 | 9.531198139 | 1041.960926 | 1.3604E−228 | 6.4718E−227 |
| Rbpj | 0.922052977 | 9.288273063 | 1040.747485 | 2.4969E−228 | 1.1846E−226 |
| Racgap1 | −1.030040933 | 9.267128908 | 1032.284027 | 1.7258E−226 | 8.1657E−225 |
| Rinl | −0.701653781 | 9.499382409 | 1031.73747 | 2.2688E−226 | 1.0706E−224 |
| Map1lc3a | 0.930943669 | 9.222575159 | 1025.770847 | 4.4945E−225 | 2.1151E−223 |
| Stk38 | −0.839920214 | 9.310126564 | 1016.59735 | 4.4323E−223 | 2.0802E−221 |
| Rom1 | −0.922814364 | 9.186853274 | 1016.525731 | 4.5941E−223 | 2.1503E−221 |
| Ier5l | 0.849227509 | 9.153032957 | 1016.270631 | 5.2197E−223 | 2.4366E−221 |
| Atic | −0.890335932 | 9.211770003 | 1015.191565 | 8.9576E−223 | 4.1703E−221 |
| Bcl2l1 | 0.976985439 | 9.276721393 | 1013.469591 | 2.1207E−222 | 9.8469E−221 |
| Abracl | −0.493515818 | 10.02337263 | 1012.64476 | 3.2046E−222 | 1.484E−220 |

TABLE 2F
IEL vs Spleen

| GeneSymbol | logFC | logCPM | LR | PValue | FDR |
|---|---|---|---|---|---|
| Rgs1 | 4.262079706 | 10.67767028 | 16605.4657 | 0 | 0 |
| Cd160 | 1.993212278 | 9.512810414 | 2102.741037 | 0 | 0 |
| Actn2 | 1.659090902 | 9.531861239 | 2089.000144 | 0 | 0 |
| Pglyrp1 | −1.501489373 | 9.640341062 | 2053.499203 | 0 | 0 |
| Gngt2 | 1.675642097 | 9.359655682 | 1985.016955 | 0 | 0 |
| Ifng | 2.245135482 | 9.611310602 | 1971.078308 | 0 | 0 |
| Atp1b3 | −1.310004856 | 9.650252258 | 1944.791465 | 0 | 0 |
| Osgin1 | 1.626820627 | 9.262214394 | 1937.431479 | 0 | 0 |
| Ubc | 0.849179414 | 10.53817088 | 1905.724403 | 0 | 0 |
| Ckb | 1.739428091 | 9.348935381 | 1902.202899 | 0 | 0 |
| Ly6c2 | −2.504398179 | 9.67510089 | 2151.306825 | 0 | 0 |
| Rps19 | −0.525105269 | 12.68187153 | 1875.342571 | 0 | 0 |
| mt-Cytb | −0.557479877 | 11.32634498 | 1739.463783 | 0 | 0 |
| Litaf | 1.571091076 | 9.323626777 | 1730.972116 | 0 | 0 |
| Lgals1 | −0.864462482 | 12.37139459 | 1689.543998 | 0 | 0 |
| Dapk2 | 1.539044461 | 9.314819002 | 1679.288972 | 0 | 0 |
| Ms4a4b | −0.585021214 | 11.67414781 | 1629.224356 | 0 | 0 |
| Emp3 | −0.828192522 | 10.36526113 | 1589.900258 | 0 | 0 |
| Rplp0 | −0.402486196 | 12.41844544 | 1541.987891 | 0 | 0 |
| Ddx5 | 0.577762999 | 10.6942535 | 1491.549434 | 0 | 0 |
| Gpr34 | 1.45129199 | 9.307758489 | 1483.078432 | 0 | 0 |
| Rps8 | −0.501436752 | 11.65645422 | 1855.710838 | 0 | 0 |
| Tspan32 | −1.857483249 | 9.43206433 | 2309.54436 | 0 | 0 |
| Ubb | 0.587733421 | 12.12717783 | 2159.371295 | 0 | 0 |
| Btg2 | 1.389291695 | 10.01747436 | 2562.865196 | 0 | 0 |
| Junb | 2.384505986 | 10.73966867 | 7545.660651 | 0 | 0 |
| Dusp1 | 3.030607317 | 9.53465407 | 4234.923152 | 0 | 0 |
| Itgb1 | −2.870641087 | 9.973925881 | 4205.16325 | 0 | 0 |
| Cd69 | 2.283758528 | 9.690665292 | 4092.761065 | 0 | 0 |
| Abi3 | 2.236724652 | 9.438058313 | 4047.605221 | 0 | 0 |
| Jund | 1.265368178 | 10.81995682 | 3897.741946 | 0 | 0 |
| Fos | 3.359701466 | 9.582648832 | 3867.81214 | 0 | 0 |
| Klf2 | −1.774396751 | 10.69289158 | 3810.169639 | 0 | 0 |
| Jun | 2.95696533 | 9.570921865 | 3765.507669 | 0 | 0 |
| Ms4a6b | −0.806571053 | 10.73301473 | 2352.116967 | 0 | 0 |
| Btg1 | 1.17808929 | 11.04741079 | 3248.989903 | 0 | 0 |
| Cxcr6 | 1.462933277 | 10.57309895 | 3323.908054 | 0 | 0 |
| Hspa1a | 3.860361274 | 9.996281107 | 3132.38968 | 0 | 0 |
| Ctsw | 1.202949028 | 10.10085922 | 2877.890061 | 0 | 0 |
| Rasgrp2 | −1.634496669 | 9.675037233 | 2872.189184 | 0 | 0 |
| Klf6 | 1.892054861 | 9.70857084 | 2829.292235 | 0 | 0 |
| Asb2 | 2.111944299 | 9.548775661 | 2817.279328 | 0 | 0 |
| Amica1 | 1.953284071 | 9.478445084 | 2779.961972 | 0 | 0 |
| Furin | 2.413436865 | 9.609122409 | 2776.783836 | 0 | 0 |
| Itm2b | 0.705576535 | 11.21870081 | 2774.794338 | 0 | 0 |
| Cd3g | 0.678489262 | 11.28334695 | 2737.378773 | 0 | 0 |
| Itgae | 2.265437232 | 9.373851416 | 3164.762104 | 0 | 0 |
| Crip1 | −0.770514594 | 12.07555511 | 1477.500162 | 0 | 0 |
| mt-Atp6 | −0.388845529 | 12.61166871 | 1444.234208 | 0 | 0 |
| S100a4 | −1.18365345 | 10.66366393 | 1435.922499 | 0 | 0 |
| Rpl8 | −0.45414365 | 11.59716841 | 1428.757788 | 0 | 0 |
| mt-Co3 | −0.393548607 | 12.29236826 | 1428.055822 | 0 | 0 |
| Skil | 1.402316776 | 9.30797085 | 1426.937262 | 0 | 0 |
| Ifi2712a | −1.450531959 | 10.18524586 | 1396.949077 | 9.6694E−306 | 3.1179E−303 |
| Gadd45b | 1.580780927 | 9.341379728 | 1381.831612 | 1.8641E−302 | 5.9015E−300 |
| Ccl4 | 3.686687264 | 10.50318841 | 1373.907492 | 9.8271E−301 | 3.0555E−298 |
| AW112010 | 0.590006061 | 11.88680588 | 1344.968361 | 1.9103E−294 | 5.8353E−292 |
| Hic1 | 1.42721533 | 9.293785776 | 1341.859244 | 9.0517E−294 | 2.7174E−291 |
| Ly6g5b | 1.413897369 | 9.280717651 | 1325.037738 | 4.0946E−290 | 1.2084E−287 |
| Bcl2a1d | 1.240479361 | 9.479817541 | 1319.618632 | 6.1637E−289 | 1.7887E−286 |
| Dusp2 | 1.29812206 | 9.739066913 | 1311.780358 | 3.1131E−287 | 8.8861E−285 |
| S1pr4 | −1.041809426 | 9.681229931 | 1297.038961 | 4.9739E−284 | 1.3969E−281 |
| Sgk1 | −1.39232095 | 9.335085016 | 1280.164904 | 2.3104E−280 | 6.3856E−278 |
| Mapkapk3 | 1.160234155 | 9.443868055 | 1274.296635 | 4.3548E−279 | 1.1848E−276 |
| Rps3a1 | −0.385150761 | 11.68169968 | 1260.878972 | 3.5881E−276 | 9.6118E−274 |
| Glipr2 | −0.940668004 | 9.853250247 | 1246.002646 | 6.1347E−273 | 1.6185E−270 |
| Rps20 | −0.56020392 | 10.99041833 | 1243.157128 | 2.5479E−272 | 6.6216E−270 |
| Rps17 | −0.563047054 | 10.69497822 | 1233.249546 | 3.6252E−270 | 9.2825E−268 |
| Malat1 | 0.588800482 | 14.37997813 | 1223.18936 | 5.5673E−268 | 1.4049E−265 |
| Tnfaip3 | 1.426715741 | 9.302203564 | 1222.117432 | 9.5192E−268 | 2.3678E−265 |
| Slamf6 | −1.452746909 | 9.424776552 | 1207.916821 | 1.1608E−264 | 2.8467E−262 |
| Lag3 | 1.863437794 | 9.404044177 | 1184.663271 | 1.3135E−259 | 3.1764E−257 |
| Rps6 | −0.367229946 | 12.18088129 | 1181.87815 | 5.2931E−259 | 1.2625E−256 |
| Bcl2a1b | 1.013192394 | 9.692981785 | 1171.176339 | 1.1209E−256 | 2.6373E−254 |
| S100a10 | −0.611743271 | 11.3818739 | 1171.100606 | 1.1641E−256 | 2.7027E−254 |
| Ehd1 | 1.245626999 | 9.349651414 | 1170.157928 | 1.8659E−256 | 4.2748E−254 |
| H2afz | −0.815169676 | 10.88644706 | 1166.361727 | 1.2472E−255 | 2.8202E−253 |
| Apol9b | 1.139074323 | 9.225731368 | 1166.140287 | 1.3933E−255 | 3.1103E−253 |
| Cd38 | 1.278780699 | 9.321520345 | 1155.58763 | 2.7384E−253 | 6.0356E−251 |
| Rpl29 | −0.451553781 | 11.21299617 | 1154.787878 | 4.0861E−253 | 8.8935E−251 |
| Rbpj | 1.213743294 | 9.354366621 | 1142.009944 | 2.4458E−250 | 5.2575E−248 |
| Gpr171 | 0.992352465 | 9.586322101 | 1112.18461 | 7.4241E−244 | 1.5764E−241 |
| Dnajb1 | 1.309635611 | 9.56298969 | 1106.132968 | 1.5343E−242 | 3.2188E−240 |
| Plac8 | 1.290227127 | 10.01229068 | 1102.413441 | 9.8705E−242 | 2.046E−239 |
| Aldoa | 0.640398084 | 10.2226702 | 1095.154589 | 3.7326E−240 | 7.6461E−238 |
| Rps5 | −0.368948183 | 12.10797403 | 1089.924265 | 5.1144E−239 | 1.0355E−236 |
| Anxa1 | −1.436448141 | 9.514591367 | 1086.543383 | 2.7773E−238 | 5.5584E−236 |
| Rgs2 | 1.44060445 | 9.304465505 | 1075.968262 | 5.522E−236 | 1.0926E−233 |
| Rgs10 | 1.055568254 | 9.634558763 | 1068.444474 | 2.3845E−234 | 4.665E−232 |
| Rplp1 | −0.407337669 | 11.58510276 | 1067.917965 | 3.1033E−234 | 6.0039E−232 |
| Rpl21 | −0.372542865 | 11.67596432 | 1061.07808 | 9.5166E−233 | 1.8209E−230 |
| Camk2n1 | 1.271116935 | 9.309540856 | 1052.321018 | 7.6181E−231 | 1.4418E−228 |
| Arhgdib | −0.41229592 | 11.15444337 | 1045.725621 | 2.0672E−229 | 3.8703E−227 |
| Neat1 | 1.195998299 | 9.446396783 | 1025.145275 | 6.1469E−225 | 1.1386E−222 |
| Rpl32 | −0.325305646 | 12.45425636 | 1010.039595 | 1.1804E−221 | 2.1635E−219 |
| Mxd1 | 1.25624995 | 9.266182111 | 1003.274001 | 3.4883E−220 | 6.327E−218 |
| mt-Nd4 | −0.510585429 | 10.65212475 | 996.4531242 | 1.0598E−218 | 1.9024E−216 |
| Ctla4 | 1.2913399 | 9.290657695 | 984.1703115 | 4.9556E−216 | 8.8048E−214 |
| Rps4x | −0.339096552 | 11.85896399 | 976.6294311 | 2.159E−214 | 3.7972E−212 |
| Hbb-bs | −1.006330094 | 9.230738885 | 976.6079899 | 2.1823E−214 | 3.7997E−212 |
| Cd3e | 0.493125143 | 10.54127341 | 975.2453323 | 4.3162E−214 | 7.441E−212 |
| Srgn | 0.536001419 | 10.69028649 | 969.6923156 | 6.9529E−213 | 1.1869E−210 |
| Rps7 | −0.361780836 | 11.89493797 | 958.9162718 | 1.5296E−210 | 2.5858E−208 |
| Smim3 | 1.114768141 | 9.249649885 | 955.3599849 | 9.0702E−210 | 1.5186E−207 |
| Fasl | 1.135893724 | 9.368313371 | 945.8583186 | 1.0545E−207 | 1.7487E−205 |
| Nr4a1 | 1.432783006 | 9.262165007 | 943.1908477 | 4.0077E−207 | 6.5831E−205 |
| Lpxn | 0.860705006 | 9.573491654 | 939.7712079 | 2.2194E−206 | 3.6117E−204 |
| Nrp1 | −1.151078008 | 9.269548869 | 922.0870285 | 1.5504E−202 | 2.4995E−200 |
| Hcst | 0.587237628 | 10.53140426 | 918.4462426 | 9.5912E−202 | 1.5321E−199 |
| Cd9 | −1.245593031 | 9.317378372 | 913.1639268 | 1.3495E−200 | 2.1361E−198 |
| Susd3 | 0.938349002 | 9.478294991 | 907.2901186 | 2.553E−199 | 4.0047E−197 |
| Rpl38 | 0.391339203 | 11.2758405 | 896.3541086 | 6.0869E−197 | 9.4629E−195 |
| Itgb7 | −0.559510469 | 10.45112407 | 894.290745 | 1.7098E−196 | 2.6346E−194 |
| Bin2 | −0.632699558 | 9.986168083 | 887.0930266 | 6.2757E−195 | 9.5853E−193 |
| Rps11 | −0.292018742 | 12.31112894 | 886.2098796 | 9.7646E−195 | 1.4784E−192 |
| Neurl3 | 1.119559655 | 9.280786296 | 858.9627424 | 8.1858E−189 | 1.2287E−186 |
| Hmgb2 | −0.947980515 | 10.87140735 | 857.9515297 | 1.358E−188 | 2.021E−186 |
| Tnfrsf25 | −1.141346675 | 9.325984856 | 852.1182469 | 2.518E−187 | 3.7155E−185 |
| Il21r | 0.94141111 | 9.484637076 | 838.9198588 | 1.8639E−184 | 2.7273E−182 |
| H3f3b | 0.374221947 | 11.35853232 | 838.6673109 | 2.1151E−184 | 3.069E−182 |
| Ccr7 | −1.230256877 | 9.274847488 | 834.9590376 | 1.3537E−183 | 1.9481E−181 |
| Sh2d1a | −0.93528822 | 9.633910801 | 834.4242155 | 1.7693E−183 | 2.5252E−181 |
| Fam65b | −1.061529155 | 9.353791259 | 831.196016 | 8.9052E−183 | 1.2606E−180 |
| Rpsa | −0.326786093 | 11.58222174 | 826.7117255 | 8.4056E−182 | 1.1803E−179 |
| S1pr1 | −1.045505474 | 9.378176807 | 823.4981118 | 4.1999E−181 | 5.8503E−179 |
| Ldlrad4 | 1.006112174 | 9.235552707 | 809.9266305 | 3.7484E−178 | 5.18E−176 |
| Prdx4 | −1.066608254 | 9.276690245 | 808.031196 | 9.6816E−178 | 1.3274E−175 |
| AB124611 | −1.023170957 | 9.268582849 | 807.8449459 | 1.0628E−177 | 1.4457E−175 |
| Klf3 | −1.186891126 | 9.31685725 | 805.2114908 | 3.9719E−177 | 5.3611E−175 |
| Nedd9 | 0.99989357 | 9.334070971 | 800.3269765 | 4.5811E−176 | 6.1359E−174 |
| Ppp1r15a | 1.114264497 | 9.318633918 | 796.3865749 | 3.2937E−175 | 4.3779E−173 |
| Ahr | 1.105381504 | 9.279326539 | 796.2018926 | 3.6128E−175 | 4.7656E−173 |
| Rps2 | −0.38282646 | 11.545051 | 795.678974 | 4.6939E−175 | 6.1451E−173 |
| Rpl35a | −0.310787517 | 11.63257095 | 791.8001781 | 3.2724E−174 | 4.2521E−172 |
| Gzmb | 1.515904822 | 9.944249279 | 782.411994 | 3.598E−172 | 4.6406E−170 |
| Cish | 0.979424054 | 9.414754089 | 780.9865643 | 7.3449E−172 | 9.4036E−170 |
| Akap13 | 0.752120035 | 9.708953687 | 778.3867251 | 2.6993E−171 | 3.4307E−169 |
| Gzma | 1.951748481 | 11.34314196 | 773.458912 | 3.1819E−170 | 4.0148E−168 |
| Gadd45g | 1.350903661 | 9.293836915 | 771.2493214 | 9.6188E−170 | 1.2049E−167 |
| Ccr5 | 1.055098668 | 9.400279713 | 767.3790788 | 6.6776E−169 | 8.305E−167 |
| Ccl5 | 1.208924468 | 13.67587951 | 757.739843 | 8.3271E−167 | 1.0283E−164 |
| Limd2 | −0.586230549 | 10.26889424 | 757.5864436 | 8.9918E−167 | 1.1026E−164 |
| Plekhf1 | 1.019330549 | 9.263192932 | 728.2887944 | 2.11E−160 | 2.5692E−158 |
| Rpl28 | −0.391130178 | 10.77653256 | 726.4014939 | 5.4284E−160 | 6.5639E−158 |
| Nkg7 | 0.578359003 | 11.62073593 | 723.3444287 | 2.5085E−159 | 3.0123E−157 |
| Sema4a | −1.025446315 | 9.351055105 | 722.8049034 | 3.2865E−159 | 3.9195E−157 |
| Rps12 | −0.454488734 | 10.7143326 | 716.5056658 | 7.7001E−158 | 9.1207E−156 |
| Mir142hg | 0.981584406 | 9.249452604 | 714.3596403 | 2.255E−157 | 2.653E−155 |
| Nr4a2 | 1.310458939 | 9.245298726 | 710.3616104 | 1.6693E−156 | 1.9507E−154 |
| Plk3 | 1.067784554 | 9.346984393 | 699.4513487 | 3.9356E−154 | 4.5685E−152 |
| Rps27a | −0.296136488 | 11.76900547 | 698.3920575 | 6.689E−154 | 7.7132E−152 |
| Bhlhe40 | 0.858136819 | 9.662074795 | 696.7934058 | 1.4894E−153 | 1.7061E−151 |
| Ms4a4c | −1.088441443 | 9.282107963 | 693.3564808 | 8.3251E−153 | 9.4743E−151 |
| mt-Nd1 | −0.380326867 | 11.2629884 | 690.4436462 | 3.5795E−152 | 4.0471E−150 |
| Med10 | 0.73641002 | 9.523002102 | 687.5656361 | 1.5124E−151 | 1.699E−149 |
| Rpl10a | −0.355092665 | 11.17378178 | 687.3757938 | 1.6632E−151 | 1.8564E−149 |
| S100a6 | −0.583209178 | 11.66425634 | 676.2798097 | 4.3047E−149 | 4.7741E−147 |
| Flna | −0.915188626 | 9.394672482 | 676.1026688 | 4.704E−149 | 5.1839E−147 |
| Slc3a2 | 0.786942874 | 9.537633493 | 675.8712633 | 5.2819E−149 | 5.7841E−147 |
| Fth1 | 0.349796278 | 10.96871293 | 669.4836567 | 1.2939E−147 | 1.4081E−145 |
| Pim1 | 0.899833541 | 9.699776734 | 669.2064071 | 1.4866E−147 | 1.6077E−145 |
| Ermn | 1.063075406 | 9.259478586 | 668.7191115 | 1.8974E−147 | 2.0394E−145 |
| Rps18 | −0.298129514 | 12.48866118 | 668.1114963 | 2.5722E−147 | 2.7476E−145 |
| Pnrc1 | 0.704253854 | 9.692827582 | 667.913303 | 2.8405E−147 | 3.0158E−145 |
| Hist1h1c | 1.172958994 | 9.29446422 | 656.8278894 | 7.3146E−145 | 7.7189E−143 |
| Pdlim1 | −0.922335931 | 9.370260078 | 656.2072833 | 9.9807E−145 | 1.0469E−142 |
| Rnf149 | 0.917040109 | 9.334081045 | 653.6098152 | 3.6648E−144 | 3.8211E−142 |
| Tff1 | 1.026000433 | 9.22546748 | 648.9038566 | 3.8681E−143 | 4.009E−141 |
| Hsd11b1 | −0.77670808 | 9.561647283 | 629.6623451 | 5.9191E−139 | 6.0985E−137 |
| Cd47 | −0.476883342 | 10.29751655 | 617.2298436 | 2.994E−136 | 3.0666E−134 |
| Rpl31 | −0.363933126 | 10.81463059 | 616.0827323 | 5.3181E−136 | 5.4151E−134 |
| Gpr18 | 0.981990926 | 9.352283484 | 615.5237916 | 7.0359E−136 | 7.1227E−134 |
| Pycard | −0.59566406 | 9.979121804 | 610.9981535 | 6.7865E−135 | 6.8305E−133 |
| Runx3 | 0.728527491 | 9.562720515 | 609.0561824 | 1.7949E−134 | 1.7961E−132 |
| Ubac2 | 0.756993118 | 9.471979592 | 604.7793569 | 1.5285E−133 | 1.5208E−131 |
| Gimap7 | 0.557513099 | 10.02409941 | 604.3949278 | 1.853E−133 | 1.8332E−131 |
| Cd27 | −1.036859444 | 9.404666177 | 603.3770562 | 3.0851E−133 | 3.0349E−131 |
| Fut7 | −0.813298011 | 9.241991725 | 601.2763096 | 8.8347E−133 | 8.6421E−131 |
| Cd7 | 1.054957536 | 9.801914175 | 598.3586942 | 3.8089E−132 | 3.705E−130 |
| Hspa8 | 0.305700865 | 11.60840881 | 597.894494 | 4.8058E−132 | 4.6488E−130 |
| Coro1a | −0.302618405 | 11.3347192 | 596.775756 | 8.4159E−132 | 8.096E−130 |
| Cdc25b | −0.926265339 | 9.328139226 | 594.1243079 | 3.1756E−131 | 3.0381E−129 |
| Gramd3 | −0.898686441 | 9.420567345 | 593.3827375 | 4.6038E−131 | 4.3804E−129 |
| Ccl3 | 2.761276819 | 9.496917967 | 588.348771 | 5.729E−130 | 5.4213E−128 |
| Nsg2 | −0.821700106 | 9.230550514 | 581.3504475 | 1.9069E−128 | 1.7948E−126 |
| Prdx6 | 0.617318162 | 9.661183975 | 580.9418509 | 2.34E−128 | 2.1905E−126 |
| Dok2 | −0.748098844 | 9.542583178 | 575.0766093 | 4.416E−127 | 4.1118E−125 |
| Apol9a | 0.687625082 | 9.203228477 | 574.0747894 | 7.2937E−127 | 6.7552E−125 |
| Mptx1 | 0.725791051 | 9.203528419 | 573.8140249 | 8.3113E−127 | 7.6569E−125 |
| Ptpn22 | 0.614248744 | 9.745489627 | 570.9953324 | 3.4104E−126 | 3.1254E−124 |
| Gm26532 | 0.863998792 | 9.218766316 | 567.0071602 | 2.5139E−125 | 2.2917E−123 |
| Adgrg1 | 0.666167406 | 9.202504636 | 561.9069435 | 3.2344E−124 | 2.9332E−122 |
| Prdx5 | 0.626969797 | 9.618053492 | 558.211872 | 2.0587E−123 | 1.8573E−121 |
| Rps3 | −0.233251504 | 11.90667401 | 557.2016632 | 3.4147E−123 | 3.0648E−121 |
| Ypel3 | 0.698378192 | 9.67572785 | 555.8482393 | 6.7262E−123 | 6.006E−121 |
| Pmepa1 | 0.722569784 | 9.209646525 | 555.5816234 | 7.6873E−123 | 6.8291E−121 |
| P2rx7 | 0.872094397 | 9.290334827 | 553.6533556 | 2.0195E−122 | 1.7849E−120 |
| Rftn1 | 0.780530727 | 9.38529654 | 553.5817803 | 2.0932E−122 | 1.8408E−120 |
| Rps15 | −0.281171407 | 11.39272392 | 550.2446875 | 1.1137E−121 | 9.7444E−120 |
| Ier2 | 0.664096741 | 9.809742381 | 549.9735323 | 1.2757E−121 | 1.1106E−119 |
| Cotl1 | 0.432498996 | 10.57561017 | 548.7207422 | 2.3894E−121 | 2.0698E−119 |
| Rpl14 | −0.278682856 | 11.83763413 | 544.6000766 | 1.8824E−120 | 1.6226E−118 |
| Id2 | 0.489582245 | 10.94720261 | 538.5213781 | 3.9546E−119 | 3.392E−117 |
| Nabp1 | 0.775579548 | 9.441457345 | 538.4457335 | 4.1073E−119 | 3.5057E−117 |
| Serpina3g | 0.932154861 | 9.583786505 | 537.5406431 | 6.4634E−119 | 5.4675E−117 |
| Ywhaq | −0.525927234 | 9.879377712 | 537.5390489 | 6.4686E−119 | 5.4675E−117 |
| mt-Nd2 | −0.534446067 | 9.939205918 | 537.222614 | 7.5797E−119 | 6.3757E−117 |
| Cpd | 0.725500446 | 9.212757368 | 536.6810278 | 9.942E−119 | 8.3226E−117 |
| Samhd1 | −0.727184214 | 9.58455645 | 535.8115532 | 1.5368E−118 | 1.2804E−116 |
| Prkcg | −0.768998828 | 9.429526956 | 533.8722419 | 4.06E−118 | 3.3663E−116 |
| Slc9a3r1 | 0.50081584 | 9.871867879 | 531.9985034 | 1.0379E−117 | 8.5651E−116 |
| Prdx1 | −0.436919808 | 10.23820402 | 515.5474221 | 3.9379E−114 | 3.2343E−112 |
| Cd2 | −0.451491441 | 10.25558839 | 514.4176199 | 6.9355E−114 | 5.6695E−112 |
| Ly6c1 | −0.845983228 | 9.223958343 | 514.1576066 | 7.9003E−114 | 6.4281E−112 |
| Rom1 | −0.874065681 | 9.273403894 | 511.8000784 | 2.5738E−113 | 2.0844E−111 |
| Aim2 | 0.8277845 | 9.261912654 | 504.4701056 | 1.0125E−111 | 8.1526E−110 |
| Sec61g | 0.400877072 | 10.22586732 | 504.4630547 | 1.016E−111 | 8.1526E−110 |
| Gstm5 | 0.902058784 | 9.230637004 | 503.3328649 | 1.7898E−111 | 1.4296E−109 |
| mt-Co2 | −0.264507091 | 11.49074591 | 502.8822201 | 2.2432E−111 | 1.7835E−109 |
| Tspo | −0.42076822 | 10.19442167 | 501.8646488 | 3.7348E−111 | 2.9559E−109 |
| Map3k8 | 0.886100204 | 9.270683592 | 501.6110699 | 4.2407E−111 | 3.3411E−109 |
| St6galnac3 | 0.883785968 | 9.281772021 | 499.8464583 | 1.0265E−110 | 8.0514E−109 |
| Ucp2 | 0.468618682 | 10.01352738 | 497.1644322 | 3.9349E−110 | 3.0724E−108 |
| Lsp1 | −0.374419062 | 10.79768635 | 495.9820381 | 7.1155E−110 | 5.531E−108 |
| Hba-a1 | −0.61069366 | 9.205576591 | 495.0296708 | 1.1466E−109 | 8.8734E−108 |
| Fgl2 | 0.806015418 | 9.503341859 | 493.6372642 | 2.3035E−109 | 1.7747E−107 |
| Efhd2 | 0.593457057 | 9.629719472 | 493.1402658 | 2.9548E−109 | 2.2665E−107 |
| Stk17b | 0.544966261 | 9.749708021 | 491.2101692 | 7.7713E−109 | 5.9348E−107 |
| Cd4 | 0.566897576 | 9.757520572 | 491.0179769 | 8.5568E−109 | 6.5062E−107 |
| 1600014C10Rik | 0.804522875 | 9.336807011 | 490.7645054 | 9.7155E−109 | 7.355E−107 |
| Cmah | −0.647892227 | 9.221071111 | 490.4164898 | 1.1566E−108 | 8.7182E−107 |
| Wbp1 | 0.78371793 | 9.336308361 | 483.5355138 | 3.6344E−107 | 2.7277E−105 |
| Smad7 | 0.824276691 | 9.295734689 | 482.9761258 | 4.81E−107 | 3.5945E−105 |
| 5430421N21Rik | 0.787547001 | 9.216296912 | 482.101492 | 7.4553E−107 | 5.5475E−105 |
| Igfbp4 | −0.890958237 | 9.223424268 | 479.0282947 | 3.4768E−106 | 2.5761E−104 |
| Rhob | 0.762632846 | 9.215115957 | 477.1124388 | 9.0798E−106 | 6.699E−104 |
| Rpl4 | −0.233195869 | 11.68632408 | 476.3582095 | 1.3249E−105 | 9.7341E−104 |
| Ccl1 | 3.115968281 | 9.596379606 | 475.9162433 | 1.6534E−105 | 1.2096E−103 |
| Gramd1a | 0.681833843 | 9.490134539 | 474.69533 | 3.0482E−105 | 2.2207E−103 |
| Prkar1a | 0.427630344 | 9.98187113 | 474.2673884 | 3.7771E−105 | 2.7403E−103 |
| Lilrb4a | 0.909524168 | 9.363990735 | 472.8669947 | 7.6189E−105 | 5.5046E−103 |
| Kcnn4 | −0.660071444 | 9.574572775 | 471.6792428 | 1.3815E−104 | 9.94E−103 |
| Rps26 | −0.315405888 | 10.92694571 | 470.3775483 | 2.6522E−104 | 1.9004E−102 |
| Isg20 | 0.825783773 | 9.282559299 | 468.9706448 | 5.3674E−104 | 3.8302E−102 |
| Tnfsf11 | 0.884737646 | 9.274816839 | 466.9953633 | 1.4441E−103 | 1.0263E−101 |
| Cd82 | 0.481854251 | 10.17845419 | 466.5620801 | 1.7943E−103 | 1.27E−101 |
| Tpm4 | −0.612729082 | 9.709350388 | 466.1516286 | 2.204E−103 | 1.5537E−101 |
| 2810474O19Rik | −0.765852699 | 9.427307461 | 464.4024391 | 5.2949E−103 | 3.7175E−101 |
| Gcnt2 | 0.670930148 | 9.208179265 | 463.4609738 | 8.4865E−103 | 5.9344E−101 |
| Dusp5 | 0.823340659 | 9.351651501 | 459.0693513 | 7.6633E−102 | 5.3373E−100 |
| Dok1 | 0.747411005 | 9.32999967 | 458.9167176 | 8.2724E−102 | 5.7386E−100 |
| Tmem256 | 0.61810974 | 9.531009831 | 458.096344 | 1.2478E−101 | 8.622E−100 |
| Adgrg5 | 0.685604528 | 9.212262974 | 457.8845661 | 1.3875E−101 | 9.5494E−100 |
| Pfdn5 | 0.361119936 | 10.44951346 | 454.7727164 | 6.5986E−101 | 4.5234E−99 |
| Traf1 | 0.665520256 | 9.518543672 | 454.3640596 | 8.0981E−101 | 5.5296E−99 |
| Rps10 | −0.264905947 | 11.16449461 | 454.1113701 | 9.1913E−101 | 6.2515E−99 |
| Itga1 | 0.74909445 | 9.228969457 | 453.097764 | 1.5274E−100 | 1.03485E−98 |
| Lilr4b | 0.858881674 | 9.344215918 | 451.2048016 | 3.9438E−100 | 2.66163E−98 |
| Rasa3 | −0.781161253 | 9.347767194 | 450.5315409 | 5.5263E−100 | 3.71524E−98 |
| Izumo1r | −1.216792535 | 9.314620339 | 448.5664961 | 1.4794E−99 | 9.90745E−98 |
| Rpl12 | −0.365836896 | 10.8499333 | 448.4935947 | 1.5344E−99 | 1.02367E−97 |
| Tnfaip8l2 | −0.656173911 | 9.525752027 | 444.3087909 | 1.24939E−98 | 8.30319E−97 |
| Txndc5 | −0.757766414 | 9.346388362 | 443.7890437 | 1.62111E−98 | 1.07326E−96 |
| Lef1 | −0.800271093 | 9.363794019 | 442.1237796 | 3.7345E−98 | 2.46307E−96 |
| Prrt1 | 0.602550339 | 9.204288164 | 440.8898295 | 6.93083E−98 | 4.55395E−96 |
| St6galnac2 | 0.639011431 | 9.20894446 | 436.5796195 | 6.00979E−97 | 3.93393E−95 |
| Tcf7 | −0.852294436 | 9.454127633 | 429.8926644 | 1.71494E−95 | 1.11837E−93 |
| Frmd4b | 0.73174728 | 9.22917364 | 425.8933346 | 1.27266E−94 | 8.26851E−93 |
| Bcl2 | −0.888178196 | 9.591081981 | 423.7626419 | 3.7023E−94 | 2.39645E−92 |
| Rpl10 | −0.249865961 | 11.48093566 | 423.7211872 | 3.78002E−94 | 2.43769E−92 |
| Penk | 1.86460897 | 9.328467588 | 423.0442881 | 5.30673E−94 | 3.40962E−92 |
| Rps24 | −0.243016167 | 11.88186655 | 420.8311132 | 1.60896E−93 | 1.02997E−91 |
| Dgka | −0.685020808 | 9.458704546 | 420.0237087 | 2.41149E−93 | 1.53805E−91 |
| Cox17 | 0.477747199 | 9.819102932 | 418.0059291 | 6.62952E−93 | 4.21289E−91 |
| Nt5e | 0.784054933 | 9.259960638 | 417.8805506 | 7.05947E−93 | 4.4698E−91 |
| Plcxd2 | 0.772699883 | 9.245478992 | 414.2485755 | 4.35845E−92 | 2.74961E−90 |
| Reep5 | −0.488697469 | 9.868713475 | 412.7987005 | 9.01418E−92 | 5.66624E−90 |
| Cd244 | 0.705384043 | 9.214900481 | 411.9645549 | 1.36929E−91 | 8.57629E−90 |
| Ifitm3 | −1.027216242 | 9.940629422 | 411.873886 | 1.43295E−91 | 8.94286E−90 |
| Arid5a | 0.713220342 | 9.354599172 | 411.1681094 | 2.04108E−91 | 1.26926E−89 |
| Myl6 | −0.261289897 | 11.30790412 | 409.3658743 | 5.03686E−91 | 3.12106E−89 |
| Ppp2r2c | 0.519156642 | 9.197405353 | 408.2335859 | 8.88447E−91 | 5.48569E−89 |
| Ly6e | −0.333786835 | 10.78770429 | 406.6496223 | 1.96528E−90 | 1.20917E−88 |
| Sike1 | −0.692535807 | 9.394852686 | 405.2866661 | 3.89146E−90 | 2.38585E−88 |
| Ndufa4 | −0.441522604 | 9.985023527 | 404.1704464 | 6.80914E−90 | 4.16003E−88 |
| 1500009L16Rik | −0.898102208 | 9.364736674 | 404.1467767 | 6.89041E−90 | 4.19496E−88 |
| Nt5c | −0.559849517 | 9.571043992 | 402.8215085 | 1.33885E−89 | 8.12269E−88 |
| Icam2 | −0.738300082 | 9.298358105 | 401.7927545 | 2.24221E−89 | 1.3556E−87 |
| Id3 | −0.987410593 | 9.302895586 | 399.6777741 | 6.47261E−89 | 3.89969E−87 |
| Znrf1 | 0.698891421 | 9.390734877 | 399.2341991 | 8.08426E−89 | 4.8539E−87 |
| Kcnab2 | −0.660175762 | 9.41152398 | 398.2712409 | 1.30998E−88 | 7.83829E−87 |
| Satb1 | −0.760996699 | 9.4527421 | 396.169245 | 3.75709E−88 | 2.24036E−86 |
| Ccdc107 | 0.654848921 | 9.378243207 | 393.4693721 | 1.45412E−87 | 8.64132E−86 |
| Add3 | −0.706199058 | 9.360336043 | 393.3945083 | 1.50972E−87 | 8.94125E−86 |
| Ppia | −0.251687631 | 11.88768862 | 392.2838879 | 2.63436E−87 | 1.5549E−85 |
| Tnf | 0.969127686 | 9.271925014 | 391.1558162 | 4.6372E−87 | 2.7278E−85 |
| Fkbp1a | −0.411230721 | 9.974773786 | 389.5576999 | 1.03316E−86 | 6.05702E−85 |
| Ppp1r16b | 0.748777981 | 9.348007448 | 389.2877898 | 1.18284E−86 | 6.91129E−85 |
| Acot7 | 0.540236341 | 9.607603124 | 387.5039543 | 2.8925E−86 | 1.68442E−84 |
| Rps25 | −0.258837917 | 11.05911713 | 387.094298 | 3.55188E−86 | 2.06151E−84 |
| Gfi1 | 0.715163437 | 9.250672945 | 386.1958488 | 5.57257E−86 | 3.22358E−84 |
| Plscr1 | 0.753523551 | 9.338606765 | 381.7694026 | 5.12551E−85 | 2.95514E−83 |
| Gm10076 | 0.602471507 | 9.555749844 | 380.8630837 | 8.07337E−85 | 4.63939E−83 |
| Ubl5 | 0.359954618 | 10.09812301 | 377.4561365 | 4.45456E−84 | 2.55141E−82 |
| Capg | 0.525034521 | 10.11973006 | 375.9622763 | 9.41997E−84 | 5.37772E−82 |
| Xcl1 | 2.334924237 | 9.473572265 | 373.6035421 | 3.07328E−83 | 1.74875E−81 |
| Hemk1 | 0.683812596 | 9.224911739 | 373.2693929 | 3.63374E−83 | 2.06094E−81 |
| Fam101b | −0.826001151 | 9.238078205 | 371.9127234 | 7.17361E−83 | 4.05542E−81 |
| Sub1 | −0.312398738 | 10.73812115 | 371.8733076 | 7.31678E−83 | 4.12297E−81 |
| Tnfrsf1b | 0.703666663 | 9.266812765 | 370.9442092 | 1.16577E−82 | 6.54784E−81 |
| Arl4c | 0.52714599 | 9.791946773 | 366.58533 | 1.03677E−81 | 5.80458E−80 |
| Hba-a2 | −0.455920224 | 9.198210536 | 366.2675731 | 1.21582E−81 | 6.78522E−80 |
| AU020206 | 0.673883407 | 9.367737735 | 365.5506469 | 1.74169E−81 | 9.68894E−80 |
| Hnrnpf | −0.273127308 | 10.67200379 | 365.2988081 | 1.97609E−81 | 1.09579E−79 |
| Hilpda | 0.741609608 | 9.26631349 | 364.3414744 | 3.19342E−81 | 1.7652E−79 |
| Il12rb2 | 0.780042435 | 9.272220666 | 363.7489147 | 4.29814E−81 | 2.36833E−79 |
| Cd200r4 | 0.745073965 | 9.245788536 | 363.332146 | 5.29697E−81 | 2.90949E−79 |
| Selm | 0.724967002 | 9.255274695 | 362.2132873 | 9.2822E−81 | 5.08244E−79 |
| Rpl13 | −0.20692553 | 12.59341642 | 358.9936129 | 4.66361E−80 | 2.54554E−78 |
| Itga4 | −0.709429771 | 9.457128938 | 355.272428 | 3.01313E−79 | 1.63952E−77 |
| Eef1b2 | −0.395983278 | 10.08532252 | 354.6347115 | 4.14843E−79 | 2.25023E−77 |
| Rps27rt | 0.40920376 | 10.6486322 | 352.6986737 | 1.09514E−78 | 5.92194E−77 |
| Zap70 | 0.480186036 | 9.677477571 | 352.1255183 | 1.45976E−78 | 7.86915E−77 |
| Rpl26 | −0.219220515 | 11.49710179 | 350.9988084 | 2.56824E−78 | 1.38019E−76 |
| Ass1 | −0.614408133 | 9.641662833 | 348.5785901 | 8.64318E−78 | 4.63062E−76 |
| Gm26740 | −0.613073552 | 9.548079775 | 348.4571538 | 9.18583E−78 | 4.90625E−76 |
| Zfp36 | 0.755036205 | 9.294838731 | 346.525669 | 2.41955E−77 | 1.28836E−75 |
| Csrnp1 | 0.702657077 | 9.254044021 | 346.1586592 | 2.90843E−77 | 1.54395E−75 |
| Cd226 | 0.652794243 | 9.387067308 | 345.9790994 | 3.18245E−77 | 1.68428E−75 |
| Gpr68 | 0.666874983 | 9.328829504 | 344.9771686 | 5.25962E−77 | 2.77517E−75 |
| Pabpc1 | 0.253543554 | 10.71757424 | 344.7679366 | 5.84144E−77 | 3.07284E−75 |
| Gm12840 | 0.891047188 | 9.457231655 | 344.4593021 | 6.81918E−77 | 3.57637E−75 |
| Cd48 | −0.38459015 | 10.43479465 | 343.3816553 | 1.17062E−76 | 6.12098E−75 |
| Sh2d2a | 0.579542848 | 9.466676588 | 343.2133641 | 1.2737E−76 | 6.64001E−75 |
| Cxcr5 | −0.530471666 | 9.20561145 | 343.1932453 | 1.28661E−76 | 6.68732E−75 |
| Il6ra | −0.569058542 | 9.20917833 | 342.7785351 | 1.58403E−76 | 8.20865E−75 |
| Rpl37a | 0.203138206 | 12.26116013 | 342.1077689 | 2.21738E−76 | 1.14567E−74 |
| Lypd8 | 0.44842715 | 9.193249939 | 340.9066075 | 4.04975E−76 | 2.08622E−74 |
| H2afy | 0.371108067 | 10.00724113 | 340.4917522 | 4.98627E−76 | 2.56109E−74 |
| Vmp1 | 0.567628617 | 9.458764331 | 339.5017466 | 8.19184E−76 | 4.19519E−74 |
| Sh3bgrl3 | 0.23283736 | 11.52323202 | 339.0176875 | 1.04424E−75 | 5.33207E−74 |
| Gzmm | −0.713492859 | 9.237177807 | 338.9103634 | 1.10198E−75 | 5.61045E−74 |
| Pld3 | 0.591215902 | 9.428245291 | 335.7969448 | 5.25095E−75 | 2.66558E−73 |
| Rpl7 | −0.20865182 | 11.19387534 | 329.5334866 | 1.21449E−73 | 6.14731E−72 |
| Sephs2 | 0.578851155 | 9.452454792 | 328.8405202 | 1.71919E−73 | 8.6767E−72 |
| Sepp1 | −0.737185666 | 9.351250863 | 328.6008691 | 1.93875E−73 | 9.75651E−72 |
| Eef1g | −0.318644983 | 10.44466423 | 328.1917319 | 2.38031E−73 | 1.19441E−71 |
| Anxa6 | −0.378854677 | 10.05966028 | 327.9775338 | 2.65025E−73 | 1.32604E−71 |
| Rps16 | −0.187940518 | 12.22909973 | 325.4524705 | 9.40299E−73 | 4.69126E−71 |
| Rhoh | 0.549715634 | 9.52013598 | 325.1010815 | 1.12151E−72 | 5.57934E−71 |
| Pelo | 0.667576923 | 9.295180382 | 324.3424209 | 1.64077E−72 | 8.13934E−71 |
| Rpl15 | −0.29061008 | 10.58474029 | 322.6470854 | 3.83987E−72 | 1.89942E−70 |
| Klrg1 | −0.895311661 | 9.277471359 | 322.4765157 | 4.18282E−72 | 2.06321E−70 |
| Tmem71 | −0.63467876 | 9.368776477 | 322.1358738 | 4.96211E−72 | 2.44069E−70 |
| Ier5 | 0.533178217 | 9.643609832 | 320.6619551 | 1.03923E−71 | 5.09723E−70 |
| Hspa5 | 0.441769561 | 9.991491978 | 320.244836 | 1.28106E−71 | 6.2657E−70 |
| Gm27166 | 0.478783135 | 9.194488904 | 319.8860088 | 1.53367E−71 | 7.48017E−70 |
| 2310001H17Rik | −0.69570124 | 9.36500569 | 319.8349174 | 1.57347E−71 | 7.65289E−70 |
| Fam3c | 0.611290084 | 9.369315527 | 319.6288024 | 1.74484E−71 | 8.46273E−70 |
| Rpl18 | −0.200629143 | 11.74785 | 317.5684137 | 4.9041E−71 | 2.37195E−69 |
| Reg3b | 0.438054199 | 9.193236754 | 314.9256552 | 1.846E−70 | 8.90373E−69 |
| Ran | −0.432537043 | 10.02805166 | 314.4525328 | 2.34041E−70 | 1.12573E−68 |
| Tesc | 0.585196699 | 9.463706763 | 310.4006473 | 1.78627E−69 | 8.56818E−68 |
| Nrgn | 0.67256668 | 9.21031371 | 310.1873651 | 1.98796E−69 | 9.50946E−68 |
| E2f2 | −0.632366672 | 9.24570481 | 309.703225 | 2.5344E−69 | 1.20901E−67 |
| Sidt1 | −0.607133596 | 9.231615429 | 309.1636563 | 3.32214E−69 | 1.58047E−67 |
| Metrnl | 0.670144523 | 9.278658789 | 307.297602 | 8.47095E−69 | 4.01897E−67 |
| Ddit4 | −0.556082538 | 9.690594056 | 307.1181298 | 9.26894E−69 | 4.38562E−67 |
| Itgb2 | −0.379932245 | 10.0524446 | 306.9472813 | 1.00983E−68 | 4.7651E−67 |
| Ptk2b | 0.589498004 | 9.37852549 | 304.9252424 | 2.78455E−68 | 1.3104E−66 |
| Stk38 | −0.601169306 | 9.410018622 | 302.5222046 | 9.29556E−68 | 4.36265E−66 |
| Krt8 | 0.394913343 | 9.191735533 | 301.4893461 | 1.56061E−67 | 7.29917E−66 |
| Asap1 | −0.713638856 | 9.28327877 | 301.4854993 | 1.56363E−67 | 7.29917E−66 |
| Glrx | 0.526069658 | 9.6245233 | 301.1671216 | 1.83441E−67 | 8.54031E−66 |
| Cd96 | 0.50746649 | 9.566029171 | 300.5855052 | 2.4559E−67 | 1.14032E−65 |
| Msn | −0.354638678 | 9.969662732 | 300.3359931 | 2.78336E−67 | 1.28893E−65 |
| Mmd | 0.683389713 | 9.258719061 | 299.6070719 | 4.01215E−67 | 1.85304E−65 |
| Rpl30 | −0.249820608 | 10.85133779 | 297.5170069 | 1.14483E−66 | 5.27347E−65 |
| Car5b | −0.612063244 | 9.237859904 | 296.8403488 | 1.60755E−66 | 7.38541E−65 |
| Crbn | 0.554618233 | 9.427304818 | 296.368566 | 2.03683E−66 | 9.33296E−65 |
| Phlda1 | 0.803113616 | 9.291148392 | 296.236558 | 2.17628E−66 | 9.94579E−65 |
| Ggt1 | −0.702663215 | 9.290810258 | 295.9132706 | 2.55948E−66 | 1.16664E−64 |
| Atic | −0.635349325 | 9.302037984 | 295.1729731 | 3.71062E−66 | 1.68693E−64 |
| Rgs16 | 0.765148476 | 9.216610114 | 293.8288353 | 7.28293E−66 | 3.30235E−64 |
| Csnk2b | 0.416324203 | 9.685639631 | 290.8795747 | 3.1982E−65 | 1.44642E−63 |
| Gm9843 | 0.381109199 | 10.19320765 | 290.8378149 | 3.26592E−65 | 1.47322E−63 |
| Rps271 | −0.43391813 | 9.777142341 | 290.2792293 | 4.3223E−65 | 1.9447E−63 |
| Iqgap2 | −0.638076071 | 9.307359719 | 289.8537682 | 5.3508E−65 | 2.40124E−63 |
| Cirbp | 0.504746243 | 9.53720808 | 289.2138674 | 7.37646E−65 | 3.30177E−63 |
| Cd200r1 | 0.672026366 | 9.260019867 | 288.664971 | 9.71515E−65 | 4.33744E−63 |
| Btla | −0.591546048 | 9.228534142 | 286.415877 | 3.00276E−64 | 1.33719E−62 |
| Phfl1b | −0.53811808 | 9.475914441 | 284.1505726 | 9.35702E−64 | 4.15624E−62 |
| Gng2 | 0.468934308 | 9.57488742 | 282.8266548 | 1.81815E−63 | 8.05538E−62 |
| Lztfl1 | 0.726684698 | 9.295833236 | 281.3215644 | 3.86907E−63 | 1.70985E−61 |
| Scn1b | 0.440541002 | 9.1964611 | 280.6666827 | 5.37422E−63 | 2.36901E−61 |
| Rpl17 | −0.180962475 | 12.17902552 | 279.7927545 | 8.33218E−63 | 3.66363E−61 |
| Tgfb1 | 0.484671853 | 9.622712466 | 279.7162695 | 8.65817E−63 | 3.79738E−61 |
| Ikzf3 | 0.655518838 | 9.312842571 | 279.698909 | 8.73392E−63 | 3.82098E−61 |
| Sdf4 | 0.413417447 | 9.734416359 | 279.5030756 | 9.63574E−63 | 4.20154E−61 |
| 2900026A02Rik | 0.349760831 | 9.191105265 | 279.4997042 | 9.65205E−63 | 4.20154E−61 |
| Calm1 | −0.231941553 | 11.07867785 | 278.0942215 | 1.9539E−62 | 8.48413E−61 |

TABLE 2G
Tissue Combined

| GeneSymbol | logFC.TissueColon_IEL | logFC.TissueColon_LPL | logFC.TissueSI_LPL |
|---|---|---|---|
| Vps37b | 0.777474128 | 3.99446302 | 3.903208385 |
| Rps8 | −0.462894732 | −0.282560786 | −0.227185564 |
| Dnajb9 | 0.242194076 | 1.230337941 | 1.179743784 |
| Gm8369 | −0.434682295 | 0.68750399 | 0.476744935 |
| Ckb | 1.683488654 | 0.985367182 | 1.041027108 |
| Nr3c1 | 0.596866537 | 1.3763776 | 1.026938489 |
| Sp100 | −0.276734925 | −0.946107553 | −0.567656721 |
| Gabpb1 | 0.40379582 | 1.330686374 | 0.891949362 |
| Uhrf2 | 0.100713404 | 1.227704361 | 0.927995577 |
| Gm8730 | −0.398463357 | −0.292225535 | 0.59358649 |
| Dtx1 | −0.612484351 | −1.44822264 | −0.207521149 |
| Areg | 0.212427582 | 1.905811086 | 1.755119712 |
| Gpr171 | 1.020738775 | 1.026142826 | 0.96946795 |
| Dusp2 | 1.26074017 | −0.285136711 | −0.04232493 |
| Cebpb | 0.417904147 | 1.289160943 | 1.492710901 |
| Snx18 | 0.253512243 | 1.181248291 | 1.402214668 |
| Lpxn | 0.960187902 | 0.964976389 | 0.97218611 |
| Atf3 | 0.221926725 | 1.430600805 | 0.663376037 |
| Plcxd2 | 0.80714267 | 1.178350458 | 1.765824897 |
| Tspo | −0.459439563 | −0.604962665 | −0.816095379 |
| Rps19 | −0.488789043 | −0.220762212 | −0.310099291 |
| Ppp1ca | −0.138260669 | −0.481445397 | −0.664694124 |
| Prkcq | −0.831805974 | −1.064405565 | −0.910979538 |
| Bcl2l11 | 0.298668059 | 1.269735942 | 1.018797786 |
| Nfkbie | 0.320540429 | 1.253990691 | 0.778292075 |
| Tnfaip8l2 | −0.656745597 | −1.002594927 | −1.249789844 |
| AB124611 | −1.071538249 | −1.025761529 | −1.069656175 |
| Rsrp1 | 0.045934577 | −0.695910085 | −0.308240957 |
| Vim | −0.426911231 | −0.60007612 | −1.201605759 |
| Zfp36l1 | 0.409767222 | 1.004959319 | 1.615568571 |
| Ninj1 | 0.456984649 | 1.330843731 | 1.146988409 |
| Fut7 | −0.902591648 | −1.053104849 | −0.949863456 |
| Gm26532 | 0.78795718 | 1.322682827 | 1.290107828 |
| Lin54 | 0.125129429 | 1.125966706 | 1.315252047 |
| Tob2 | 0.373497726 | 1.295279865 | 1.335778732 |
| Stx11 | 0.108734331 | 1.219878433 | 0.820456389 |
| Cxcl10 | 0.238497963 | 1.594973302 | 1.451352068 |
| Rpl37 | 0.116065648 | 0.27929211 | 0.479609638 |
| Malat1 | 0.589352269 | 0.540521994 | 0.566513255 |
| Tmsb4x | 0.034205633 | −0.250963133 | −0.32512786 |
| Wdr1 | −0.110386912 | −0.7976657 | 0.69312976 |
| Plscr1 | 0.820629431 | 1.375621367 | 0.946173857 |
| Rpl36 | 0.109936658 | 0.304016116 | 0.591038405 |
| Relb | 0.452701151 | 1.315929558 | 1.088010985 |
| Ehd1 | 1.350600781 | 1.355200628 | 1.225979164 |
| Rps28 | −0.052612178 | 0.164343875 | 0.733603222 |
| Hsp90ab1 | 0.183973249 | 0.770065508 | 0.382334631 |
| Anxa6 | −0.440796803 | −0.777608644 | −1.033157811 |
| Map3k8 | 0.786413742 | 1.533242075 | 1.57543773 |
| Hist1h1c | 1.156393093 | 1.914039852 | 1.713398295 |
| Clic1 | −0.126278872 | −0.577454964 | −0.698476892 |
| Ddit4 | −0.550655713 | −1.322882165 | −1.549423159 |
| AW112010 | 0.530350631 | −0.269167964 | −0.120512172 |
| Rbm39 | 0.121133079 | 0.591211818 | 0.666508922 |
| Actn2 | 1.674186971 | 1.09423599 | 1.668343285 |
| Sema4a | −1.076586289 | −1.381910022 | −1.47235225 |
| S1pr4 | −1.055213166 | −1.004407804 | −1.132415542 |
| Tnfrsf1b | 0.839325851 | 1.485819835 | 1.360630531 |
| Anxa1 | −1.511107054 | −1.396051355 | −2.188471733 |
| Rpl41 | 0.089207305 | 0.269891062 | 0.458372809 |
| Dennd4a | 0.40921547 | 1.450236431 | 1.196022302 |
| Emb | 0.256860695 | 1.031210454 | 0.639730557 |
| Sub1 | −0.212606624 | 0.525046364 | 0.171178095 |
| Arl4c | −0.49415572 | −0.985244078 | −1.582854063 |
| Smim3 | 1.157858029 | 1.41368162 | 1.507860613 |
| Ifng | 2.239791472 | 1.729179922 | 1.492569488 |
| Rgcc | 0.283384363 | 1.767101847 | 0.447220961 |
| Psmb8 | −0.110504023 | −0.489140738 | −0.649175005 |
| Satb1 | −0.73170529 | 0.829100852 | 0.63424116 |
| mt-Cytb | −0.546265334 | −0.42351736 | −0.415001765 |
| Dusp10 | 0.166031321 | 1.460807234 | 1.313007697 |
| Osgin1 | 1.638804775 | 0.665375907 | 1.581835883 |
| Vcpkmt | 0.485718496 | 1.353540366 | 1.181968319 |
| Chd2 | 0.490638634 | 1.209409986 | 1.442340463 |
| Ifi27l2a | −1.447651542 | −1.320038979 | −1.014727105 |
| Cnbp | 0.328954303 | 0.714682188 | 0.750969488 |
| Gngt2 | 1.6715869 | 1.008170978 | 1.394249117 |
| Ptpn7 | 0.034473291 | −1.133069364 | −0.919157917 |
| Herpud1 | 0.052692615 | 1.177557859 | 1.034451055 |
| Nrp1 | −1.191469771 | −1.264227267 | −1.279785482 |
| Gch1 | 0.60986345 | 1.540707807 | 0.845457397 |
| Birc3 | 0.178272259 | 1.269097181 | 1.31937574 |
| Cfl1 | −0.181428647 | −0.482315806 | −0.478158167 |
| Vgll4 | 0.553934184 | 1.336407518 | 0.803366983 |
| Kcnn4 | −0.66182234 | −1.259249871 | −1.318323926 |
| Kpna1 | 0.236709891 | 1.160296596 | 1.200898507 |
| Nabp1 | 0.898469347 | 1.13108023 | 0.937505709 |
| Hsph1 | 0.063771251 | 1.223646137 | 0.804849095 |
| Il22 | 0.636407037 | 1.727603551 | 3.375767379 |
| Lrrc58 | −0.280678767 | −0.931657812 | 0.078861966 |
| Polg2 | 0.115967235 | 0.988832852 | 0.963823755 |
| Lck | −0.046016642 | −0.478587291 | −0.328836522 |
| Rps27l | −0.422405737 | −0.706969497 | −1.151277553 |
| 4930523C07Rik | −0.049553342 | 0.77439766 | 0.458814483 |
| Capg | 0.444179609 | −0.258086072 | −1.063350641 |
| Rbpj | 1.221814752 | 0.931669327 | 0.810911354 |
| Aldoa | 0.602520735 | 0.466664486 | −0.004097069 |
| Rps17 | −0.554098377 | −0.38055098 | −0.294626154 |
| Zc3hav1 | −0.118281001 | 0.800121918 | 0.686580555 |
| Ddit3 | 0.633412182 | 1.056768321 | 1.505656004 |
| Camk2n1 | 1.281680699 | 1.103759494 | 0.695796302 |
| Il2rg | 0.199050169 | 0.585724189 | 0.878628642 |
| Traf4 | 0.387903813 | 1.143370115 | 1.166481487 |
| Hnrnpf | −0.289637392 | −0.457763526 | −0.442403466 |
| Ppp1r16b | 0.722505925 | 1.131785945 | 1.123510124 |
| Usmg5 | 0.338211483 | 0.562446426 | 0.884243935 |
| Gm9493 | −0.373348956 | −0.119724093 | 0.654836138 |
| Gm20186 | 0.376387595 | 0.983081489 | 1.508057083 |
| Cyba | −0.12164775 | −0.398081178 | −0.628053762 |
| Zeb1 | 0.268976051 | 1.117792672 | 0.955359161 |
| Gm9843 | 0.050245934 | 0.036828943 | 0.933575173 |
| Rpl27 | 0.112631873 | 0.224950706 | 0.508803925 |
| Dusp4 | 0.153431151 | 0.94029633 | 0.821545552 |
| Tnfrsf9 | 0.954876882 | 1.509800497 | 0.514373324 |
| PhIda1 | 0.741010016 | 1.447979137 | 1.083660668 |
| Map3k14 | 0.236317904 | 0.977429196 | 1.3068537 |
| Slamf6 | −1.45895434 | −0.965084691 | −0.811078956 |
| Ctla2a | 0.298799669 | 1.071932508 | 0.251123192 |
| Hdac1 | 0.284581969 | −0.596423747 | −0.759024583 |
| H2-Q4 | 0.275988306 | 0.749276391 | 1.06798585 |
| Fam46a | 0.080223304 | 1.343132414 | 0.596687066 |
| Fam65b | −1.112352618 | −0.915624467 | −0.779034525 |
| Mxi1 | 0.327724833 | 1.096423847 | 0.916698704 |
| Ccr5 | 1.136442096 | 1.03663238 | 1.414443159 |
| D8Ertd738e | 0.149173189 | 0.591554745 | 0.705970327 |
| Gzmb | 1.484474865 | 1.671588876 | 1.899825501 |
| Sdf4 | 0.478973842 | 0.771120166 | 0.376506356 |
| Stat3 | 0.590121867 | 1.132951528 | 0.779233082 |
| Zyx | −0.565107863 | −1.017829067 | −1.105422427 |
| Ms4a4c | −1.135742662 | −1.080990165 | −1.214454415 |
| Hic1 | 1.490225264 | 0.64937241 | 1.158758155 |
| Rpl23a-ps3 | −0.108683292 | −0.042050175 | 0.797803645 |
| Hbp1 | 0.153253653 | 1.045263235 | 1.08918818 |
| Ywhaq | −0.50728297 | −0.695383339 | −0.933175471 |
| Dtx4 | 0.325907203 | 0.900111631 | 1.402409997 |
| Prdx1 | −0.441253622 | −0.579820321 | −0.84275194 |
| Dapk2 | 1.504802415 | 0.556414029 | 1.01750907 |
| Rpl35 | −0.198257565 | −0.137039195 | 0.501566337 |
| Tgif2 | 0.028216662 | 1.039982456 | 0.913047919 |
| Aebp2 | 0.532530932 | 1.101390751 | 1.10384383 |
| Spn | −0.442218027 | −0.924628279 | −0.908993083 |
| Peli1 | 0.180571514 | 0.945055781 | 0.778179164 |
| Rplp0 | −0.40462238 | −0.178016 | −0.146562691 |
| Insig1 | 0.334913953 | 1.102843857 | 1.570247867 |
| Arpc3 | 0.335449112 | 0.581367164 | 0.431387866 |
| Hk2 | 0.400462873 | 1.272037672 | 0.895345873 |
| Arl6ip5 | −0.350457803 | −0.638658603 | −0.860140937 |
| Reep5 | −0.543838571 | −0.696314196 | −0.957208206 |
| Gpx4 | 0.416949548 | 0.78623455 | 0.456449996 |
| Prdx4 | −1.087776303 | −1.023745704 | −1.086234685 |
| mt-Co3 | −0.373726515 | −0.283800873 | −0.245313316 |
| Slc38a2 | 0.027794305 | 0.890981228 | 1.265084578 |
| Fkbp1a | −0.457711587 | −0.609776872 | −0.807238893 |
| Cdc42se1 | −0.509406321 | −0.94083351 | −0.723317952 |
| Mfsd6 | 0.322475507 | 1.177204896 | 0.993936731 |
| Hbb-bs | −0.951439469 | −0.833727323 | −0.920809369 |
| Sgk1 | −1.401626324 | −0.881994243 | −1.153947764 |
| Ccr9 | 0.472499792 | 0.23501735 | 2.246312713 |
| Cdc42 | −0.194978171 | −0.457363489 | −0.492606763 |
| Nr4a3 | 0.37734512 | 1.000912689 | 0.932683465 |
| Sash3 | 0.284465257 | −0.904496449 | −0.450998844 |
| Flna | −1.021845631 | −0.972657753 | −0.939784581 |
| Id2 | 0.546829646 | −0.150446648 | −0.594648468 |
| Ccl4 | 3.615166269 | 0.52950705 | 1.537818194 |
| Sik1 | 0.187430647 | 1.003588487 | 1.049624518 |
| Ldlrad4 | 0.983465129 | 1.170317969 | 1.242760762 |
| Sh3bgrl3 | 0.178502582 | −0.151124267 | −0.520462292 |
| Selk | 0.20817301 | 0.633800345 | 0.574745819 |
| Txndc5 | −0.793923149 | −1.079044363 | −1.044409331 |
| Lztfl1 | 0.811919165 | 1.025717051 | 1.778608941 |
| Lilrb4a | 1.040771731 | 1.662603733 | 1.34866513 |
| Plac8 | 1.367181977 | 1.647105676 | 1.336533536 |
| Pfn1 | −0.227374524 | −0.464331976 | −0.494146426 |
| Got1 | 0.912228134 | 2.351604957 | 2.113401784 |
| Lgals1 | −0.94241438 | −0.994539931 | −1.811527329 |
| Coro1a | −0.383867449 | −0.878811184 | −0.690027583 |
| Neurl3 | 1.260230617 | 2.259242074 | 2.263083272 |
| Bcl2a1d | 1.325205435 | 1.991992964 | 2.161429488 |
| Fosl2 | 0.686981772 | 2.317777656 | 2.162692641 |
| Ddx5 | 0.655299575 | 0.933006654 | 1.022505971 |
| Mxd1 | 1.185545347 | 2.408175848 | 2.46113118 |
| Prdx6 | 0.739133202 | 1.540245599 | 1.651132315 |
| Btg2 | 1.340461972 | 1.610387778 | 1.855311255 |
| Zc3h12a | 0.368012894 | 2.196963947 | 1.920529072 |
| Nr4a1 | 1.42026219 | 2.585195396 | 2.900870393 |
| Fth1 | 0.438172042 | 0.958382191 | 0.695953321 |
| Kdm6b | 0.591919262 | 2.601159919 | 2.643867444 |
| Ms4a6b | −0.855435822 | −1.113772966 | −1.061157866 |
| Gpr132 | 0.493626692 | 1.832550927 | 1.916165176 |
| Dgat1 | 1.068086006 | 2.507427706 | 2.268265285 |
| P2ry10 | 0.323139521 | 1.961409192 | 2.046154573 |
| Hspa8 | 0.262418528 | 0.947116547 | 0.507076538 |
| Hif1a | 0.506541402 | 1.846412062 | 1.394299692 |
| Emp3 | −0.885092139 | −1.135130594 | −1.534190065 |
| Cytip | 0.137241836 | 1.150444566 | 1.050113634 |
| Arhgap31 | 0.592761886 | 1.828812646 | 2.147009927 |
| Cd160 | 1.958868733 | 0.090626817 | 1.530097888 |
| Ubc | 0.781082329 | 1.059189076 | 1.267041913 |
| Rora | 0.393843317 | 1.626184495 | 1.580189974 |
| Arf4 | 0.450172863 | 1.33901057 | 1.4270623 |
| Dnajb1 | 1.088486132 | 2.625921173 | 1.768476477 |
| S100a4 | −1.381786387 | −1.735446939 | −2.192760959 |
| Slc3a2 | 0.924990309 | 1.747063943 | 2.041745429 |
| Skil | 1.356501548 | 2.046292284 | 2.167743506 |
| Tspan32 | −1.855941613 | −1.830468648 | −1.826575438 |
| Gadd45b | 1.644895671 | 2.363959806 | 2.524371898 |
| Cd69 | 2.140083096 | 1.914092308 | 2.039146629 |
| Lilr4b | 0.990097521 | 2.072444849 | 1.943112929 |
| Itgb2 | −0.437930531 | −1.311953723 | −1.34504361 |
| Rasgrp2 | −1.64073261 | −1.468891321 | −1.529423898 |
| Hilpda | 0.905718851 | 2.266685919 | 2.255743112 |
| Klf2 | −1.908273266 | −1.798692995 | −1.640237406 |
| Itgb7 | −0.626689284 | −1.091031288 | −1.049129185 |
| Samsn1 | 0.78733775 | 1.881432949 | 1.370388308 |
| Nfkbiz | 0.564142293 | 2.33024327 | 2.303550906 |
| Cd48 | −0.503647963 | −1.580728968 | −1.440014098 |
| Crem | 1.206942842 | 3.204559128 | 2.776895991 |
| Tnfrsf4 | 0.711364773 | 2.978807129 | 2.192236669 |
| Hspa5 | 0.512591422 | 1.816140229 | 1.534632246 |
| Ifngr1 | 0.1883354 | 1.834862227 | 1.306178775 |
| Ms4a4b | −0.69284433 | −1.322205103 | −1.664468636 |
| Pim1 | 0.795270671 | 2.633332305 | 2.675211977 |
| Pnrc1 | 0.753615753 | 2.106610657 | 2.294488941 |
| Odc1 | 0.740493817 | 3.345533836 | 2.810015439 |
| Jund | 1.182643946 | 2.186035889 | 1.805102708 |
| Ctla4 | 1.41332301 | 3.559115658 | 3.332369379 |
| Bhlhe40 | 0.657224846 | 2.50015099 | 2.34894059 |
| H3f3b | 0.477447103 | 1.445573521 | 1.329294416 |
| Junb | 2.293137211 | 2.821816343 | 3.206316002 |
| Btg1 | 1.136233966 | 2.049380574 | 2.322550341 |
| Tnfaip3 | 1.367360021 | 3.427648559 | 3.788144571 |
| Rgs1 | 4.407235959 | 3.620285869 | 4.048113626 |
| Ubald2 | 0.135059491 | 2.902661389 | 2.820329823 |
| Dusp5 | 0.88846288 | 2.589658521 | 2.324825039 |
| Tgif1 | 0.340947728 | 2.662628566 | 2.532311028 |
| Eif1 | 0.243600213 | 0.897018313 | 0.883631546 |
| Gramd3 | −0.887373725 | 2.001685574 | 1.518112402 |
| Ramp3 | 0.522452694 | 2.929582726 | 2.693749522 |
| Orai1 | 0.427837319 | 2.054256414 | 1.840750897 |
| Cd52 | −0.037974717 | −0.76219941 | −0.92083237 |
| Srgn | 0.650406948 | 1.163382845 | 0.975668862 |
| Akap13 | 0.784905453 | 1.650364325 | 1.687843023 |
| Traf1 | 0.731922421 | 1.939073251 | 1.667369064 |
| Dusp1 | 2.876266732 | 3.102439229 | 3.283532744 |
| Nfkbia | 0.475871735 | 2.098426093 | 1.964656983 |
| Pde4b | 0.340023511 | 2.135521921 | 2.032281316 |
| Il21r | 1.066352442 | 2.30984097 | 2.07051372 |
| Bcl2a1b | 1.159629853 | 2.026583358 | 2.045369986 |
| Rgs2 | 1.759347044 | 3.276603612 | 2.690971945 |
| Itgb1 | −2.939748656 | −2.801847036 | −3.211007959 |
| Icos | 0.760296669 | 2.291146199 | 2.058091574 |
| Prr7 | 0.576910072 | 2.642494452 | 2.148843937 |
| Gna13 | 0.539301733 | 2.190136992 | 2.212428937 |
| Ubb | 0.608762321 | 1.16229746 | 0.888058795 |
| Ifrd1 | −0.341562044 | 2.123182298 | 2.008949468 |
| Furin | 2.373523387 | 3.208611369 | 3.210149442 |
| Lmna | 0.355749856 | 2.240959029 | −0.139418188 |
| Ctsd | −0.140445912 | −1.256042585 | −1.271899886 |
| Dnaja1 | 0.065582453 | 1.228590453 | 0.7971197 |
| Crip1 | −0.848292102 | −0.618979571 | −1.41225136 |
| Gpr183 | −0.01113956 | 1.374005606 | 1.168714694 |
| Rrad | 0.234847579 | 1.697004089 | 1.594487107 |
| Ccnd3 | −0.329577306 | −1.091466059 | −1.193102261 |
| Nmrk1 | 0.428665057 | 1.738810544 | 1.410635343 |
| Rps29 | −0.118044035 | 0.078118241 | 0.675561481 |
| Itgae | 2.118239644 | 0.501790147 | 1.613848439 |
| Rps27rt | 0.111778933 | 0.348355098 | 1.256433873 |
| Dnajb6 | 0.42215896 | 1.048311667 | 0.903790169 |
| Ptprcap | −0.333296008 | −0.877052246 | −0.657134231 |
| Itm2b | 0.681017998 | 0.052557376 | 0.053940026 |
| Maff | 0.050178399 | 1.358068789 | 1.717191704 |
| Coq10b | 0.487474641 | 1.48815124 | 1.590649725 |
| Nr4a2 | 1.446906214 | 2.322415055 | 2.418160579 |
| Rnf157 | 0.220493355 | 1.530141868 | 1.533565628 |
| Csrnp1 | 0.705386191 | 1.723900036 | 1.723469654 |
| Asb2 | 2.113128181 | 1.044679174 | 1.30745449 |
| Fosb | 0.448314652 | 1.939752943 | 1.173309182 |
| Per1 | 0.223435351 | 1.324161399 | 1.655894131 |
| Tnfsf8 | 0.591517515 | 2.049539473 | 1.756714493 |
| Jun | 2.701679209 | 1.602621976 | 2.540136604 |
| Rpl37a | 0.189109219 | 0.337383963 | 0.57738162 |
| Ppia | −0.260468097 | −0.515647244 | −0.498914699 |
| Actr3 | −0.264798485 | −0.678531948 | −0.608192443 |
| Ets1 | 0.308769926 | 0.854380244 | 0.897921106 |
| Tbc1d10c | −0.086644317 | −1.048383269 | −0.603012764 |
| Ctsw | 1.133912136 | 0.290339316 | 0.428742181 |
| Rac2 | −0.090279627 | −0.457497646 | −0.519640137 |
| Hspe1 | 0.125328811 | 1.183289599 | 0.848573905 |
| Cirbp | 0.671161615 | 1.276126719 | 1.325610584 |
| Nrip1 | 0.295798404 | 1.495372547 | 1.599803514 |
| Amica1 | 1.912537743 | 0.540771353 | 0.63720074 |
| Tnfsf11 | 1.000192602 | 2.011665838 | 1.063992968 |
| Ifnar1 | 0.410475913 | 1.357726581 | 1.453941357 |
| Cd3g | 0.649613891 | 0.161480653 | 0.074462457 |
| Kdm2b | 0.35110053 | 1.500408169 | 1.365103199 |
| Litaf | 1.724751835 | 1.533159899 | 1.755054093 |
| Nfkb2 | 0.309083464 | 1.45267526 | 1.126281499 |
| Cnn2 | −0.125436839 | −0.833705916 | −0.656443942 |
| Crbn | 0.689883175 | 1.357746765 | 1.315931184 |
| Hsd11b1 | −0.845495767 | −1.422255063 | −1.515881372 |
| Fasl | 1.24195807 | 1.680829553 | 1.40489363 |
| Myl6 | −0.303122474 | −0.557407151 | −0.743323701 |
| Tnfrsf18 | 0.51027686 | 1.418821878 | 0.897476585 |
| Sh2d2a | 0.683649276 | 1.523838419 | 1.534011293 |
| S100a6 | −0.701943112 | −0.925981609 | −1.558846918 |
| Bin2 | −0.716008243 | −1.168500998 | −0.929495808 |
| Arhgdib | −0.47819901 | −0.678544906 | −0.662983446 |
| Abi3 | 2.169958211 | 0.784928318 | 1.430075624 |
| Ppp1r15a | 1.012213891 | 1.834038062 | 2.123484272 |
| Serpinb9 | −0.332079489 | 1.762867925 | 1.404007136 |
| Cdkn1a | 0.659293662 | 2.26693393 | 1.978030213 |
| Itk | 0.717407706 | 1.534128909 | 1.605916535 |
| Glipr2 | −1.012290006 | −1.288705972 | −1.608611968 |
| Pglyrp1 | −1.560081733 | −1.527481278 | −1.707966652 |
| Fam110a | 0.441701934 | 1.956309955 | 1.205559948 |
| S100a10 | −0.686684425 | −0.755853614 | −1.196982129 |
| Ptp4a1 | 0.423511875 | 1.501139578 | 1.631998685 |
| Arid5a | 0.615734799 | 1.671003212 | 1.588627125 |
| Hsp90aa1 | 0.265232836 | 1.516738352 | 0.86998671 |
| Fos | 3.307489836 | 1.662604426 | 2.60078825 |
| Il4ra | 0.262122295 | 1.768613781 | 1.41559797 |
| Gm12840 | 0.852894278 | 2.038425581 | 2.604479224 |
| Lsp1 | −0.469255726 | −0.871230904 | −0.802596375 |
| Rel | 0.328871591 | 1.654583385 | 1.684956995 |
| Rpl38 | 0.304384053 | 0.391714781 | 0.80557411 |
| Pycard | −0.68605811 | −1.204678987 | −1.095961376 |
| Cxcr6 | 1.481967857 | 0.614235458 | 0.242141429 |
| Zfp36l2 | −0.069112161 | 1.097355571 | 1.827830136 |
| Ptpn22 | 0.637640602 | 1.105479795 | 1.407606827 |
| AI467606 | −0.359100183 | −1.428559988 | −1.619259014 |
| Sqstm1 | 0.312316129 | 1.45874511 | 1.187923862 |
| D16Ertd472e | 0.528778168 | 1.560727657 | 1.355501147 |
| Ly6c2 | −2.635976447 | −2.243628333 | −2.730896442 |
| Hspa1a | 3.317587579 | 4.024832432 | 3.280867889 |
| Atp1b3 | −1.338905265 | −1.163595102 | −1.260436923 |
| Srsf5 | 0.318943246 | 1.058847246 | 1.077088912 |
| Rps27 | 0.105449775 | 0.336978206 | 0.811170641 |
| Stk17b | 0.539893656 | 1.082520549 | 1.239142099 |
| Cd28 | 0.068398706 | 1.095601812 | 0.97109367 |
| Rnf125 | −0.044505337 | 1.467264431 | 1.164227533 |
| Clk1 | 0.561911434 | 1.338154822 | 1.645145128 |
| Klf6 | 1.842955526 | 1.439880235 | 1.966650811 |
| Apobec3 | −0.366808091 | −0.852090391 | −0.527398995 |
| Ndufa4 | −0.442428302 | −0.680313916 | −0.62851769 |
| Apol9b | 1.129514917 | 0.060106654 | 0.431228775 |
| Tmem123 | 0.395783307 | 0.858468607 | 0.762317881 |
| Pim3 | 0.156477935 | 1.049132589 | 1.270764685 |
| Rpl39 | −0.003885791 | 0.14733345 | 0.492421372 |
| Adora2a | 0.327386179 | 1.093504821 | 0.814533896 |
| Id3 | −0.983976649 | −1.278212723 | −1.408714151 |
| Abcb1b | 0.084811939 | 1.03652624 | 0.878461297 |
| Calm1 | −0.195447541 | −0.347331831 | −0.60335137 |
| Cdc25b | −1.001522994 | −0.97877959 | −1.052446445 |
| Phf11b | −0.595465154 | −0.962170945 | −0.832403232 |
| Arpc1b | −0.172988304 | −0.41263938 | −0.36702828 |
| Btg3 | 0.40302651 | 0.954493008 | 1.329566612 |
| Socs3 | 0.534617386 | 1.276515071 | 1.205826408 |
| Gpr34 | 1.406180781 | 0.523866618 | 0.752660178 |
| Cd7 | 0.935530259 | −0.713133698 | 0.512262464 |
| Capn2 | −0.394029971 | −0.888329339 | −1.003188684 |
| Pde4d | 0.245207794 | 0.967356097 | 0.893857778 |
| Nfil3 | 0.397653866 | 1.106103131 | 0.792415119 |
| Gimap6 | 0.349265811 | 0.588644836 | 0.727785154 |
| Ly6g5b | 1.33303203 | 0.485097242 | 0.157396087 |
| mt-Atp6 | −0.360413265 | −0.197400255 | −0.170326369 |
| Lfng | −0.266671791 | −0.820206738 | −1.30945717 |
| Avl9 | 0.413463318 | 0.992356488 | 1.138418506 |
| Esyt1 | −0.466108154 | −0.899494199 | −0.850901825 |
| Itpkb | 0.167612218 | 0.953070885 | 0.909004332 |
| Irf4 | 0.02354066 | 0.928322708 | 0.868943333 |
| Mir142hg | 1.033642023 | 0.963245615 | 1.048769847 |
| Nkg7 | 0.53133988 | −0.169812636 | −0.218528706 |
| Zcchc11 | 0.416794184 | 0.991651408 | 1.111287695 |
| Prkarla | 0.498190887 | 0.547270984 | 0.36703625 |
| Def6 | −0.079903039 | −0.845247189 | −0.627240921 |
| Slc9a3r1 | 0.490368769 | −0.270993821 | −0.330351807 |

| GeneSymbol | logCPM | LR | PValue | FDR |
|---|---|---|---|---|
| Vps37b | 10.03454595 | 30072.7125 | 0 | 0 |
| Rps8 | 11.62809538 | 2038.340013 | 0 | 0 |
| Dnajb9 | 9.316221247 | 2038.542663 | 0 | 0 |
| Gm8369 | 9.792018318 | 2044.58434 | 0 | 0 |
| Ckb | 9.342944532 | 2055.399452 | 0 | 0 |
| Nr3c1 | 9.352262661 | 2059.924855 | 0 | 0 |
| Sp100 | 9.725036992 | 2060.348785 | 0 | 0 |
| Gabpb1 | 9.366278776 | 2073.287426 | 0 | 0 |
| Uhrf2 | 9.439369633 | 2077.350399 | 0 | 0 |
| Gm8730 | 10.38863469 | 2087.887702 | 0 | 0 |
| Dtx1 | 9.344984765 | 2098.163291 | 0 | 0 |
| Areg | 9.282241434 | 2108.862759 | 0 | 0 |
| Gpr171 | 9.650575449 | 2109.203412 | 0 | 0 |
| Dusp2 | 9.611670263 | 2127.669358 | 0 | 0 |
| Cebpb | 9.3841575 | 2133.511667 | 0 | 0 |
| Snx18 | 9.268008725 | 2190.232089 | 0 | 0 |
| Lpxn | 9.6426486 | 2193.717339 | 0 | 0 |
| Atf3 | 9.235797229 | 2208.396751 | 0 | 0 |
| Plcxd2 | 9.299070121 | 2035.025508 | 0 | 0 |
| Tspo | 10.07426107 | 2023.345914 | 0 | 0 |
| Rps19 | 12.63595929 | 2023.3203 | 0 | 0 |
| Ppp1ca | 10.49158341 | 1994.14525 | 0 | 0 |
| Prkcq | 9.361161976 | 1853.113558 | 0 | 0 |
| Bcl2l11 | 9.267192159 | 1854.01069 | 0 | 0 |
| Nfkbie | 9.322134913 | 1856.618069 | 0 | 0 |
| Tnfaip8l2 | 9.437338541 | 1861.9971 | 0 | 0 |
| AB124611 | 9.228809043 | 1871.487619 | 0 | 0 |
| Rsrp1 | 10.20843239 | 1878.782142 | 0 | 0 |
| Vim | 10.67445805 | 1885.643262 | 0 | 0 |
| Zfp36l1 | 9.441704346 | 1885.761429 | 0 | 0 |
| Ninj1 | 9.293255705 | 2231.202495 | 0 | 0 |
| Fut7 | 9.205232517 | 1929.797777 | 0 | 0 |
| Gm26532 | 9.255741547 | 1938.00365 | 0 | 0 |
| Lin54 | 9.261013077 | 1938.45873 | 0 | 0 |
| Tob2 | 9.34461902 | 1940.459028 | 0 | 0 |
| Stx11 | 9.356175668 | 1942.830244 | 0 | 0 |
| Cxcl10 | 9.271930887 | 1943.092733 | 0 | 0 |
| Rpl37 | 11.92011245 | 1950.728079 | 0 | 0 |
| Malat1 | 14.51807182 | 1976.820212 | 0 | 0 |
| Tmsb4x | 14.40631533 | 1989.761718 | 0 | 0 |
| Wdr1 | 9.737409412 | 1932.290417 | 0 | 0 |
| Plscr1 | 9.425543981 | 1845.833759 | 0 | 0 |
| Rpl36 | 11.71243313 | 2232.517819 | 0 | 0 |
| Relb | 9.371248744 | 2289.99945 | 0 | 0 |
| Ehd1 | 9.407112899 | 2500.528557 | 0 | 0 |
| Rps28 | 11.51231021 | 2511.446233 | 0 | 0 |
| Hsp90ab1 | 10.9175943 | 2531.925683 | 0 | 0 |
| Anxa6 | 9.925148315 | 2535.31738 | 0 | 0 |
| Map3k8 | 9.344670944 | 2540.995063 | 0 | 0 |
| Hist1h1c | 9.414339905 | 2569.222047 | 0 | 0 |
| Clic1 | 10.5597468 | 2577.83152 | 0 | 0 |
| Ddit4 | 9.550516634 | 2600.936733 | 0 | 0 |
| AW112010 | 11.67788795 | 2611.060111 | 0 | 0 |
| Rbm39 | 10.50344179 | 2642.465618 | 0 | 0 |
| Actn2 | 9.546273651 | 2644.093081 | 0 | 0 |
| Sema4a | 9.283140537 | 2653.703924 | 0 | 0 |
| S1pr4 | 9.586568308 | 2654.674252 | 0 | 0 |
| Tnfrsf1b | 9.355129694 | 2660.647695 | 0 | 0 |
| Anxa1 | 9.411481213 | 2691.011076 | 0 | 0 |
| Rpl41 | 12.6407868 | 2731.32137 | 0 | 0 |
| Dennd4a | 9.274100293 | 2760.713271 | 0 | 0 |
| Emb | 10.0275267 | 2499.689705 | 0 | 0 |
| Sub1 | 10.94059551 | 2495.827025 | 0 | 0 |
| Arl4c | 9.658613942 | 2472.060488 | 0 | 0 |
| Smim3 | 9.29654546 | 2450.775194 | 0 | 0 |
| Ifng | 9.645960024 | 2313.756922 | 0 | 0 |
| Rgcc | 9.423330348 | 2323.950455 | 0 | 0 |
| Psmb8 | 10.64253999 | 2337.404757 | 0 | 0 |
| Satb1 | 9.601014895 | 2341.400318 | 0 | 0 |
| mt-Cytb | 11.21803568 | 2343.700873 | 0 | 0 |
| Dusp10 | 9.359033873 | 2356.458156 | 0 | 0 |
| Osgin1 | 9.252826914 | 2372.802143 | 0 | 0 |
| Vcpkmt | 9.367243143 | 2393.600427 | 0 | 0 |
| Chd2 | 9.393875664 | 2268.146622 | 0 | 0 |
| Ifi27l2a | 10.02903758 | 2393.718927 | 0 | 0 |
| Cnbp | 10.09284839 | 2394.16337 | 0 | 0 |
| Gngt2 | 9.361328315 | 2400.498408 | 0 | 0 |
| Ptpn7 | 9.466595096 | 2419.001687 | 0 | 0 |
| Herpud1 | 9.439096692 | 2425.533038 | 0 | 0 |
| Nrp1 | 9.223298204 | 2432.20787 | 0 | 0 |
| Gch1 | 9.354399974 | 2433.304066 | 0 | 0 |
| Birc3 | 9.33752681 | 2433.841201 | 0 | 0 |
| Cfl1 | 11.32725331 | 2444.786376 | 0 | 0 |
| Vgll4 | 9.587844265 | 2393.91655 | 0 | 0 |
| Kcnn4 | 9.466905719 | 2772.34088 | 0 | 0 |
| Kpna1 | 9.300137202 | 1843.522866 | 0 | 0 |
| Nabp1 | 9.532328268 | 1838.067205 | 0 | 0 |
| Hsph1 | 9.370438221 | 1557.836398 | 0 | 0 |
| Il22 | 9.338583329 | 1562.121908 | 0 | 0 |
| Lrrc58 | 9.493596476 | 1562.433427 | 0 | 0 |
| Polg2 | 9.233059976 | 1579.366602 | 0 | 0 |
| Lck | 10.49845031 | 1579.753574 | 0 | 0 |
| Rps27l | 9.674913084 | 1579.872913 | 0 | 0 |
| 4930523C07Rik | 9.699182127 | 1581.475882 | 0 | 0 |
| Capg | 9.985868123 | 1585.265244 | 0 | 0 |
| Rbpj | 9.368575331 | 1587.924302 | 0 | 0 |
| Aldoa | 10.22924571 | 1589.244945 | 0 | 0 |
| Rps17 | 10.6596725 | 1592.755036 | 0 | 0 |
| Zc3hav1 | 9.554454222 | 1593.51781 | 0 | 0 |
| Ddit3 | 9.316873219 | 1601.418493 | 0 | 0 |
| Camk2n1 | 9.323686292 | 1602.078899 | 0 | 0 |
| Il2rg | 10.00219656 | 1605.322872 | 0 | 0 |
| Traf4 | 9.292948329 | 1608.521941 | 0 | 0 |
| Hnrnpf | 10.55525903 | 1613.128498 | 0 | 0 |
| Ppp1r16b | 9.413801897 | 1557.800865 | 0 | 0 |
| Usmg5 | 9.936350613 | 1552.872437 | 0 | 0 |
| Gm9493 | 10.23217758 | 1549.335257 | 0 | 0 |
| Gm20186 | 9.253161772 | 1546.807571 | 0 | 0 |
| Cyba | 10.60431895 | 1498.529546 | 0 | 0 |
| Zeb1 | 9.301751944 | 1502.294376 | 0 | 0 |
| Gm9843 | 10.33749646 | 1503.691737 | 0 | 0 |
| Rpl27 | 11.33055291 | 1504.195501 | 0 | 0 |
| Dusp4 | 9.213500374 | 1507.49956 | 0 | 0 |
| Tnfrsf9 | 9.321390476 | 1509.427303 | 0 | 0 |
| PhIda1 | 9.361394132 | 1509.496196 | 0 | 0 |
| Map3k14 | 9.283321913 | 1519.537609 | 0 | 0 |
| Slamf6 | 9.377252261 | 1615.425761 | 0 | 0 |
| Ctla2a | 10.09921699 | 1523.777018 | 0 | 0 |
| Hdac1 | 9.546612696 | 1527.943772 | 0 | 0 |
| H2-Q4 | 9.630172513 | 1528.27877 | 0 | 0 |
| Fam46a | 9.394476103 | 1532.18059 | 0 | 0 |
| Fam65b | 9.310020493 | 1535.116406 | 0 | 0 |
| Mxi1 | 9.258459389 | 1535.331101 | 0 | 0 |
| Ccr5 | 9.453526865 | 1545.929884 | 0 | 0 |
| D8Ertd738e | 9.906338542 | 1546.031392 | 0 | 0 |
| Gzmb | 10.14452997 | 1546.525056 | 0 | 0 |
| Sdf4 | 9.821782432 | 1526.727595 | 0 | 0 |
| Stat3 | 9.515695899 | 1840.239522 | 0 | 0 |
| Zyx | 9.42338924 | 1618.184248 | 0 | 0 |
| Ms4a4c | 9.23739877 | 1629.796583 | 0 | 0 |
| Hic1 | 9.283045422 | 1737.143544 | 0 | 0 |
| Rpl23a-ps3 | 10.57494605 | 1741.679055 | 0 | 0 |
| Hbp1 | 9.340839113 | 1751.790252 | 0 | 0 |
| Ywhaq | 9.77397317 | 1756.913129 | 0 | 0 |
| Dtx4 | 9.223206978 | 1775.308624 | 0 | 0 |
| Prdx1 | 10.11192982 | 1776.398305 | 0 | 0 |
| Dapk2 | 9.293084109 | 1780.844891 | 0 | 0 |
| Rpl35 | 11.13718458 | 1785.683581 | 0 | 0 |
| Tgif2 | 9.23223275 | 1790.943013 | 0 | 0 |
| Aebp2 | 9.382936752 | 1810.668654 | 0 | 0 |
| Spn | 9.544899055 | 1812.185322 | 0 | 0 |
| Peli1 | 9.632602802 | 1812.607057 | 0 | 0 |
| Rplp0 | 12.40493661 | 1817.429013 | 0 | 0 |
| Insig1 | 9.305878553 | 1819.439028 | 0 | 0 |
| Arpc3 | 10.3928013 | 1822.685843 | 0 | 0 |
| Hk2 | 9.240976423 | 1830.502099 | 0 | 0 |
| Arl6ip5 | 9.930300727 | 1836.898892 | 0 | 0 |
| Reep5 | 9.767497293 | 1733.322052 | 0 | 0 |
| Gpx4 | 9.948950125 | 1731.797924 | 0 | 0 |
| Prdx4 | 9.235537849 | 1731.056056 | 0 | 0 |
| mt-Co3 | 12.20719178 | 1713.852782 | 0 | 0 |
| Slc38a2 | 9.380152268 | 1635.802681 | 0 | 0 |
| Fkbp1a | 9.869140443 | 1647.111254 | 0 | 0 |
| Cdc42se1 | 9.485319255 | 1658.571945 | 0 | 0 |
| Mfsd6 | 9.299958076 | 1663.612597 | 0 | 0 |
| Hbb-bs | 9.200640448 | 1664.72306 | 0 | 0 |
| Sgk1 | 9.293049535 | 1668.770669 | 0 | 0 |
| Ccr9 | 9.24920942 | 1669.561887 | 0 | 0 |
| Cdc42 | 10.55125063 | 1672.292717 | 0 | 0 |
| Nr4a3 | 9.219687388 | 1624.091677 | 0 | 0 |
| Sash3 | 9.392380929 | 1680.800024 | 0 | 0 |
| Flna | 9.339442013 | 1690.430613 | 0 | 0 |
| Id2 | 10.81654571 | 1695.187398 | 0 | 0 |
| Ccl4 | 10.14764842 | 1696.147403 | 0 | 0 |
| Sik1 | 9.224860652 | 1703.135534 | 0 | 0 |
| Ldlrad4 | 9.264750802 | 1703.376687 | 0 | 0 |
| Sh3bgrl3 | 11.4029623 | 1704.291179 | 0 | 0 |
| Selk | 9.923482712 | 1709.896179 | 0 | 0 |
| Txndc5 | 9.289175725 | 1710.771391 | 0 | 0 |
| Lztfl1 | 9.35467745 | 1684.358705 | 0 | 0 |
| Lilrb4a | 9.500994059 | 2799.837328 | 0 | 0 |
| Plac8 | 10.29800915 | 2666.923532 | 0 | 0 |
| Pfn1 | 12.40959764 | 2823.18042 | 0 | 0 |
| Got1 | 9.572501168 | 6155.123374 | 0 | 0 |
| Lgals1 | 12.08066005 | 6170.117639 | 0 | 0 |
| Coro1a | 11.10558954 | 6173.409649 | 0 | 0 |
| Neurl3 | 9.445974691 | 6174.776117 | 0 | 0 |
| Bcl2a1d | 9.713856957 | 6253.274041 | 0 | 0 |
| Fosl2 | 9.360613737 | 6382.142761 | 0 | 0 |
| Ddx5 | 10.94756817 | 6396.297438 | 0 | 0 |
| Mxd1 | 9.428217948 | 6638.119592 | 0 | 0 |
| Prdx6 | 9.932199951 | 6693.369054 | 0 | 0 |
| Btg2 | 10.27784938 | 6722.847197 | 0 | 0 |
| Zc3h12a | 9.375963011 | 6767.694681 | 0 | 0 |
| Nr4a1 | 9.441853787 | 6805.260947 | 0 | 0 |
| Fth1 | 11.28479407 | 6872.446213 | 0 | 0 |
| Kdm6b | 9.499428313 | 6926.302094 | 0 | 0 |
| Ms4a6b | 10.51149168 | 7027.371621 | 0 | 0 |
| Gpr132 | 9.625285306 | 7072.34788 | 0 | 0 |
| Dgat1 | 9.520162453 | 7113.950101 | 0 | 0 |
| P2ry10 | 9.409785965 | 6070.471392 | 0 | 0 |
| Hspa8 | 11.98283195 | 6067.230307 | 0 | 0 |
| Hif1a | 9.566468789 | 5800.541484 | 0 | 0 |
| Emp3 | 10.15792608 | 5634.940442 | 0 | 0 |
| Cytip | 9.939689328 | 4728.057313 | 0 | 0 |
| Arhgap31 | 9.474258321 | 4796.907361 | 0 | 0 |
| Cd160 | 9.433702154 | 2814.080819 | 0 | 0 |
| Ubc | 10.79352095 | 4856.266946 | 0 | 0 |
| Rora | 9.735274165 | 4955.577014 | 0 | 0 |
| Arf4 | 9.74527555 | 4956.839167 | 0 | 0 |
| Dnajb1 | 9.951049781 | 4962.650222 | 0 | 0 |
| S100a4 | 10.35643749 | 5050.816426 | 0 | 0 |
| Slc3a2 | 9.817091287 | 7244.608843 | 0 | 0 |
| Skil | 9.427601511 | 5070.343816 | 0 | 0 |
| Tspan32 | 9.333183118 | 5088.9466 | 0 | 0 |
| Gadd45b | 9.530669537 | 5134.595901 | 0 | 0 |
| Cd69 | 9.782614588 | 5148.722666 | 0 | 0 |
| Lilr4b | 9.559633455 | 5221.957907 | 0 | 0 |
| Itgb2 | 9.853071355 | 5228.958886 | 0 | 0 |
| Rasgrp2 | 9.546231995 | 5314.353412 | 0 | 0 |
| Hilpda | 9.465091188 | 5456.24451 | 0 | 0 |
| Klf2 | 10.40246321 | 5566.904066 | 0 | 0 |
| Itgb7 | 10.24576431 | 5083.968747 | 0 | 0 |
| Samsn1 | 9.573511029 | 4684.830967 | 0 | 0 |
| Nfkbiz | 9.434908969 | 7264.143413 | 0 | 0 |
| Cd48 | 10.15245869 | 7549.34267 | 0 | 0 |
| Crem | 9.682165373 | 11246.04429 | 0 | 0 |
| Tnfrsf4 | 10.12383994 | 11439.35552 | 0 | 0 |
| Hspa5 | 10.52876384 | 11491.98875 | 0 | 0 |
| Ifngr1 | 10.6249228 | 12241.35701 | 0 | 0 |
| Ms4a4b | 11.32479865 | 12696.72714 | 0 | 0 |
| Pim1 | 10.31487793 | 14315.81915 | 0 | 0 |
| Pnrc1 | 10.16392663 | 14881.26408 | 0 | 0 |
| Odc1 | 9.952419577 | 15414.75011 | 0 | 0 |
| Jund | 11.36341037 | 15875.17087 | 0 | 0 |
| Ctla4 | 9.751318648 | 16157.30951 | 0 | 0 |
| Bhlhe40 | 10.24123106 | 18383.23837 | 0 | 0 |
| H3f3b | 11.89130548 | 18628.63053 | 0 | 0 |
| Junb | 11.37526503 | 19488.80684 | 0 | 0 |
| Btg1 | 11.71813077 | 19540.27276 | 0 | 0 |
| Tnfaip3 | 9.733547635 | 19674.48034 | 0 | 0 |
| Rgs1 | 10.88182319 | 21795.32212 | 0 | 0 |
| Ubald2 | 10.08974357 | 22204.07143 | 0 | 0 |
| Dusp5 | 9.681302178 | 11216.17781 | 0 | 0 |
| Tgif1 | 9.475989105 | 10855.11363 | 0 | 0 |
| Eif1 | 11.52939519 | 10657.53 | 0 | 0 |
| Gramd3 | 9.833476646 | 10324.26187 | 0 | 0 |
| Ramp3 | 9.589354936 | 7595.304751 | 0 | 0 |
| Orai1 | 9.620559436 | 7682.097248 | 0 | 0 |
| Cd52 | 12.265481 | 7737.734437 | 0 | 0 |
| Srgn | 11.04442682 | 7769.393006 | 0 | 0 |
| Akap13 | 10.01433481 | 8164.656794 | 0 | 0 |
| Traf1 | 9.817235232 | 8193.623841 | 0 | 0 |
| Dusp1 | 9.732110949 | 8513.455523 | 0 | 0 |
| Nfkbia | 10.38534176 | 8552.271703 | 0 | 0 |
| Pde4b | 9.456656488 | 7281.724662 | 0 | 0 |
| Il21r | 9.854712698 | 8723.721998 | 0 | 0 |
| Bcl2a1b | 10.05711684 | 8913.380572 | 0 | 0 |
| Rgs2 | 9.702426281 | 8964.070432 | 0 | 0 |
| Itgb1 | 9.705679139 | 9226.334266 | 0 | 0 |
| Icos | 9.895747212 | 9291.702058 | 0 | 0 |
| Prr7 | 9.545379603 | 9695.382535 | 0 | 0 |
| Gna13 | 9.680943589 | 9786.483787 | 0 | 0 |
| Ubb | 12.53331081 | 9989.288582 | 0 | 0 |
| Ifrd1 | 9.693003713 | 10167.64578 | 0 | 0 |
| Furin | 10.01245811 | 8904.797941 | 0 | 0 |
| Lmna | 9.487124604 | 4658.20846 | 0 | 0 |
| Ctsd | 9.949305296 | 4845.431139 | 0 | 0 |
| Dnaja1 | 10.42008114 | 4631.648037 | 0 | 0 |
| Crip1 | 11.90464273 | 3132.064265 | 0 | 0 |
| Gpr183 | 9.513344586 | 3141.680794 | 0 | 0 |
| Rrad | 9.39537213 | 3149.5252 | 0 | 0 |
| Ccnd3 | 9.736841478 | 3152.424211 | 0 | 0 |
| Nmrk1 | 9.349533747 | 3167.947491 | 0 | 0 |
| Rps29 | 12.07015461 | 3191.395691 | 0 | 0 |
| Itgae | 9.329438603 | 3234.931065 | 0 | 0 |
| Rps27rt | 10.90517839 | 3265.713412 | 0 | 0 |
| Dnajb6 | 9.840231332 | 3283.455427 | 0 | 0 |
| Ptprcap | 10.30898425 | 3310.884109 | 0 | 0 |
| Itm2b | 11.11270922 | 3315.950892 | 0 | 0 |
| Maff | 9.240293623 | 3337.823207 | 0 | 0 |
| Coq10b | 9.389551852 | 3344.97829 | 0 | 0 |
| Nr4a2 | 9.378053303 | 4644.481088 | 0 | 0 |
| Rnf157 | 9.270523021 | 3363.842169 | 0 | 0 |
| Csrnp1 | 9.355377363 | 3381.74524 | 0 | 0 |
| Asb2 | 9.517846942 | 3394.318362 | 0 | 0 |
| Fosb | 9.303641607 | 3126.413381 | 0 | 0 |
| Per1 | 9.256131905 | 3124.848247 | 0 | 0 |
| Tnfsf8 | 9.718612422 | 3082.301398 | 0 | 0 |
| Jun | 9.585203942 | 3082.055112 | 0 | 0 |
| Rpl37a | 12.4341219 | 2829.244733 | 0 | 0 |
| Ppia | 11.75036121 | 2843.993406 | 0 | 0 |
| Actr3 | 10.45956272 | 2849.847024 | 0 | 0 |
| Ets1 | 10.064916 | 2856.422455 | 0 | 0 |
| Tbc1d10c | 9.730725723 | 2875.702927 | 0 | 0 |
| Ctsw | 10.03070607 | 2876.03227 | 0 | 0 |
| Rac2 | 11.32889337 | 2877.122189 | 0 | 0 |
| Hspe1 | 9.993360632 | 2881.352973 | 0 | 0 |
| Cirbp | 9.674191382 | 3397.589205 | 0 | 0 |
| Nrip1 | 9.435157115 | 2943.750441 | 0 | 0 |
| Amica1 | 9.41423544 | 2949.790876 | 0 | 0 |
| Tnfsf11 | 9.406688169 | 2954.591251 | 0 | 0 |
| Ifnar1 | 9.421114289 | 2964.121212 | 0 | 0 |
| Cd3g | 11.2059335 | 2977.399621 | 0 | 0 |
| Kdm2b | 9.339281333 | 2987.835357 | 0 | 0 |
| Litaf | 9.380464881 | 3000.045609 | 0 | 0 |
| Nfkb2 | 9.395165675 | 3024.090259 | 0 | 0 |
| Cnn2 | 10.16482738 | 3076.591106 | 0 | 0 |
| Crbn | 9.56278392 | 2946.210161 | 0 | 0 |
| Hsd11b1 | 9.443353968 | 3412.966153 | 0 | 0 |
| Fasl | 9.476220888 | 3349.320794 | 0 | 0 |
| Myl6 | 11.14840445 | 3511.339172 | 0 | 0 |
| Tnfrsf18 | 9.841475941 | 4250.584103 | 0 | 0 |
| Sh2d2a | 9.653507549 | 4255.040768 | 0 | 0 |
| S100a6 | 11.39787222 | 3469.674452 | 0 | 0 |
| Bin2 | 9.826963154 | 4264.487697 | 0 | 0 |
| Arhgdib | 10.97661097 | 4277.750305 | 0 | 0 |
| Abi3 | 9.391339833 | 4283.327994 | 0 | 0 |
| Ppp1r15a | 9.451226916 | 4287.130131 | 0 | 0 |
| Serpinb9 | 9.595001511 | 4348.90624 | 0 | 0 |
| Cdkn1a | 9.482496831 | 4408.038615 | 0 | 0 |
| Itk | 9.614921991 | 4410.719519 | 0 | 0 |
| Glipr2 | 9.696046944 | 4420.494789 | 0 | 0 |
| Pglyrp1 | 9.506947739 | 4470.0937 | 0 | 0 |
| Fam110a | 9.361980417 | 4511.411134 | 0 | 0 |
| S100a10 | 11.19773491 | 4516.927676 | 0 | 0 |
| Ptp4a1 | 9.525742489 | 4529.394015 | 0 | 0 |
| Arid5a | 9.50033819 | 4544.040212 | 0 | 0 |
| Hsp90aa1 | 10.36821572 | 4593.064023 | 0 | 0 |
| Fos | 9.552464487 | 4202.563514 | 0 | 0 |
| Il4ra | 9.363165505 | 4128.939871 | 0 | 0 |
| Gm12840 | 9.824287347 | 4262.194424 | 0 | 0 |
| Lsp1 | 10.59943054 | 3987.168317 | 0 | 0 |
| Rel | 9.330686608 | 3532.720938 | 0 | 0 |
| Rpl38 | 11.47201227 | 3553.915854 | 0 | 0 |
| Pycard | 9.8090997 | 3566.116382 | 0 | 0 |
| Cxcr6 | 10.49165114 | 4030.459915 | 0 | 0 |
| Zfp36l2 | 9.621046058 | 3625.420712 | 0 | 0 |
| Ptpn22 | 9.913891288 | 3635.182121 | 0 | 0 |
| AI467606 | 9.43007721 | 3640.142865 | 0 | 0 |
| Sqstm1 | 9.498888294 | 3650.157793 | 0 | 0 |
| D16Ertd472e | 9.472104236 | 3737.310203 | 0 | 0 |
| Ly6c2 | 9.502687689 | 3618.463071 | 0 | 0 |
| Hspa1a | 10.53633121 | 3787.129231 | 0 | 0 |
| Atp1b3 | 9.546244885 | 3800.287392 | 0 | 0 |
| Srsf5 | 9.897879383 | 3863.410621 | 0 | 0 |
| Rps27 | 13.00250321 | 3869.553469 | 0 | 0 |
| Stk17b | 9.912060431 | 3906.395949 | 0 | 0 |
| Cd28 | 10.12482917 | 3764.96956 | 0 | 0 |
| Rnf125 | 9.417495282 | 3924.712406 | 0 | 0 |
| Clk1 | 9.595487126 | 3970.918448 | 0 | 0 |
| Klf6 | 9.797177616 | 3972.157002 | 0 | 0 |
| Apobec3 | 9.640320763 | 1495.459668 | 0 | 0 |
| Ndufa4 | 9.876365917 | 1492.897357 | 0 | 0 |
| Apol9b | 9.199371767 | 1487.707525 | 0 | 0 |
| Tmem123 | 9.564567543 | 1487.503495 | 0 | 0 |
| Pim3 | 9.272489284 | 1486.441633 | 0 | 0 |
| Rpl39 | 11.64815567 | 1484.740228 | 0 | 0 |
| Adora2a | 9.348672319 | 1483.853226 | 0 | 0 |
| Id3 | 9.247020127 | 1483.833186 | 0 | 0 |
| Abcb1b | 9.244761887 | 1482.289748 | 0 | 0 |
| Calm1 | 10.97692159 | 1481.737386 | 0 | 0 |
| Cdc25b | 9.279842463 | 1481.142685 | 0 | 0 |
| Phf11b | 9.40303954 | 1474.921118 | 0 | 0 |
| Arpc1b | 10.71697209 | 1472.676489 | 0 | 0 |
| Btg3 | 9.276956785 | 1467.968361 | 0 | 0 |
| Socs3 | 9.39717008 | 1464.828407 | 0 | 0 |
| Gpr34 | 9.284399972 | 1464.226552 | 0 | 0 |
| Cd7 | 9.668408289 | 1454.523736 | 0 | 0 |
| Capn2 | 9.422824606 | 1450.935801 | 0 | 0 |
| Pde4d | 9.22996209 | 1445.702756 | 0 | 0 |
| Nfil3 | 9.237942693 | 1431.837424 | 0 | 0 |
| Gimap6 | 10.30711133 | 1428.814757 | 0 | 7.8975E−308 |
| Ly6g5b | 9.254627632 | 1426.806364 | 0 | 2.1484E−307 |
| mt-Atp6 | 12.54893107 | 1421.705249 | 5.7448E−308 | 2.7405E−306 |
| Lfng | 9.501444391 | 1418.204835 | 3.3025E−307 | 1.5711E−305 |
| Avl9 | 9.328579122 | 1409.911793 | 2.0816E−305 | 9.8758E−304 |
| Esyt1 | 9.431724645 | 1407.121941 | 8.3901E−305 | 3.9698E−303 |
| Itpkb | 9.419678004 | 1402.3989 | 8.8845E−304 | 4.1923E−302 |
| Irf4 | 9.221609323 | 1396.730424 | 1.5089E−302 | 7.1006E−301 |
| Mir142hg | 9.267707121 | 1391.820647 | 1.754E−301 | 8.232E−300 |
| Nkg7 | 11.44730108 | 1387.945816 | 1.2157E−300 | 5.6904E−299 |
| Zcchc11 | 9.302440385 | 1383.018269 | 1.4258E−299 | 6.6559E−298 |
| Prkarla | 10.04221526 | 1363.674016 | 2.2468E−295 | 1.046E−293 |
| Def6 | 9.460419037 | 1358.115422 | 3.6117E−294 | 1.677E−292 |
| Slc9a3r1 | 9.774883184 | 1355.051977 | 1.6689E−293 | 7.7286E−292 |

Table 3A-3Y. Tables related to the 10×, single-cell sequencing on T cells derived from the in-vivo mouse experiments originating from the small intestine or colonic lamina propria. Table 3A-X Contains the results of 1 vs all differential expression tests for each of the 12 clusters defined in the study. Additionally, contains the results of within-cluster, between-genotype differential expression comparisons. Table 3Y—GWAS.

Table Description and Headings:

Table 3B-3X: Each section contains the output from an edgeR likelihood ratio test

LPL-X vs. All: Differential expression between cells of cluster LPL-X and the remaining cells. Positive log FC values are associated with genes with higher expression in LPL-X than in the remaining clusters.

LPL-X Ctrl vs. KG: Differential expression between the Ctrl cells of cluster LPL-X and the KG cells of cluster LPL-X. Positive log FC values are associated with genes with higher expression Ctrl cells.

Table 3Y: LogFC and FDR are from the respective LPL-X Ctrl vs. KG data. GWAS?represents whether or not the gene is associated with an JBD GWAS loci

TABLE 3A
LPL-0 vs. All

| GeneSymbol | logFC | logCPM | LR | PValue | FDR |
|---|---|---|---|---|---|
| Kdm6b | 2.107587482 | 9.740581499 | 2670.288539 | 0 | 0 |
| Actb | 1.497848932 | 13.40498915 | 2396.01558 | 0 | 0 |
| Pim1 | 1.461379632 | 10.82584099 | 2720.504688 | 0 | 0 |
| Pdcd4 | 1.219186897 | 9.736968363 | 1010.192462 | 1.0935E−221 | 4.4192E−218 |
| Ptpn22 | 0.952379409 | 10.09353484 | 1004.31626 | 2.0705E−220 | 6.6942E−217 |
| Cdkn1b | 1.406854085 | 9.41746112 | 924.3880645 | 4.9003E−203 | 1.3203E−199 |
| Skil | 1.075200413 | 9.558821973 | 630.5081997 | 3.8752E−139 | 8.9495E−136 |
| Insig1 | 1.236303319 | 9.37347705 | 609.4877932 | 1.446E−134 | 2.9219E−131 |
| Cbx3 | 1.138162421 | 9.518041852 | 583.4355315 | 6.711E−129 | 1.2054E−125 |
| Ppp1r16b | 1.071177085 | 9.488149826 | 577.2586691 | 1.4804E−127 | 2.3932E−124 |
| Abhd2 | 1.264504281 | 9.292308214 | 557.0948216 | 3.6024E−123 | 5.2943E−120 |
| Kmt2d | 1.182934835 | 9.264164205 | 501.3129014 | 4.9239E−111 | 6.6333E−108 |
| Malat1 | 0.469300018 | 14.66860135 | 468.8149754 | 5.8028E−104 | 7.216E−101 |
| Notch2 | 1.144895562 | 9.219647316 | 460.7171162 | 3.3561E−102 | 3.8753E−99 |
| Srrm2 | 0.728412434 | 9.808238582 | 450.9545447 | 4.4708E−100 | 4.81828E−97 |
| Ric1 | 1.070534111 | 9.254655607 | 415.4351764 | 2.40462E−92 | 2.42957E−89 |
| B4galt1 | 0.929978748 | 9.375488034 | 378.7315027 | 2.35036E−84 | 2.23505E−81 |
| Gm2a | 0.806274736 | 9.581878937 | 372.8559359 | 4.47069E−83 | 4.01518E−80 |
| Gnb1 | 0.683710369 | 9.703253309 | 339.4692154 | 8.32657E−76 | 7.0846E−73 |
| Akap13 | 0.490064243 | 10.3088199 | 317.9684284 | 4.0126E−71 | 3.24338E−68 |
| Birc6 | 0.899837946 | 9.320978822 | 316.6681692 | 7.70296E−71 | 5.92981E−68 |
| Ncoa3 | 0.950672833 | 9.252526077 | 311.806689 | 8.82375E−70 | 6.48385E−67 |
| Furin | 0.768132247 | 10.38141056 | 304.7209557 | 3.08504E−68 | 2.16838E−65 |
| Actg1 | −0.386910949 | 12.09926089 | 303.0075558 | 7.2868E−68 | 4.90827E−65 |
| Klf13 | 0.673649384 | 9.695536973 | 286.8107826 | 2.46304E−64 | 1.5927E−61 |
| Alkbh5 | 0.7944529 | 9.368743909 | 269.8374031 | 1.23125E−60 | 7.65554E−58 |
| Marf1 | 0.860276436 | 9.233752654 | 237.0707291 | 1.71172E−53 | 1.02488E−50 |
| Slc39a1 | 0.870689481 | 9.215151083 | 236.7856356 | 1.97515E−53 | 1.14037E−50 |
| Acot2 | 0.85369456 | 9.215557552 | 228.5059564 | 1.26233E−51 | 7.03684E−49 |
| Rpgrip1 | 0.891339331 | 9.229068972 | 226.3087385 | 3.80516E−51 | 2.05047E−48 |
| Lgals1 | −0.482229612 | 11.62762181 | 224.1163784 | 1.14428E−50 | 5.96725E−48 |
| S100a10 | −0.401281287 | 10.93996309 | 224.0265794 | 1.19707E−50 | 6.04744E−48 |
| Arglu1 | 0.754488666 | 9.336509872 | 221.4240554 | 4.42349E−50 | 2.16697E−47 |
| Srsf2 | 0.517320857 | 9.857790838 | 219.0310508 | 1.47143E−49 | 6.99619E−47 |
| Dpysl2 | 0.723664118 | 9.405651172 | 217.5019153 | 3.17171E−49 | 1.46497E−46 |
| S100a4 | −0.950902667 | 9.86649183 | 208.771962 | 2.54561E−47 | 1.14312E−44 |
| Atxn1 | 0.741510873 | 9.344913237 | 202.2253919 | 6.82676E−46 | 2.98274E−43 |
| Zc3hav1 | 0.547715562 | 9.665242259 | 200.5016307 | 1.62318E−45 | 6.90533E−43 |
| Klf2 | −1.069853137 | 9.96079822 | 200.3107523 | 1.78657E−45 | 7.40554E−43 |
| Supt16 | 0.80168198 | 9.269926089 | 199.557353 | 2.60872E−45 | 1.05432E−42 |
| Crip1 | −0.533283322 | 11.66663236 | 199.1348964 | 3.22566E−45 | 1.27185E−42 |
| Btg1 | 0.311293881 | 12.25006794 | 188.8750979 | 5.59599E−43 | 2.15392E−40 |
| Myh9 | 0.651340239 | 9.438500102 | 187.5898867 | 1.06764E−42 | 4.01382E−40 |
| Emp3 | −0.572932793 | 9.864609149 | 179.6697432 | 5.72175E−41 | 2.10222E−38 |
| Pitpnc1 | 0.634559946 | 9.3795869 | 168.1865931 | 1.84181E−38 | 6.6166E−36 |
| S100a6 | −0.529021515 | 10.99619486 | 159.8843786 | 1.19925E−36 | 4.21459E−34 |
| Ppm1h | 0.63807851 | 9.32382984 | 154.0799487 | 2.22463E−35 | 7.65177E−33 |
| Erdr1 | 0.702265193 | 9.351150867 | 153.7127232 | 2.67615E−35 | 9.01305E−33 |
| H2afz | −0.426974884 | 10.7655434 | 153.4276703 | 3.08892E−35 | 1.01909E−32 |
| Cdk6 | 0.69278665 | 9.323662785 | 148.8770447 | 3.05082E−34 | 9.86392E−32 |
| Whsc1l1 | 0.512329382 | 9.540585951 | 145.903876 | 1.36256E−33 | 4.31904E−31 |
| Jund | 0.351175103 | 11.8341681 | 144.7390714 | 2.4491E−33 | 7.61388E−31 |
| Mbnl1 | 0.413036368 | 9.849897993 | 142.1924147 | 8.82692E−33 | 2.69238E−30 |
| Rbm15 | 0.628984036 | 9.221090464 | 133.4475927 | 7.21627E−31 | 2.16034E−28 |
| Vim | −0.437802323 | 10.47337793 | 131.8303156 | 1.62971E−30 | 4.79015E−28 |
| Pan3 | 0.626656499 | 9.234208712 | 129.8623289 | 4.39208E−30 | 1.2679E−27 |
| Btg2 | 0.352738759 | 10.53793205 | 129.3740822 | 5.6169E−30 | 1.59303E−27 |
| Tmf1 | 0.627601139 | 9.275192923 | 129.1549672 | 6.27249E−30 | 1.74829E−27 |
| 2810417H13Rik | −1.247677001 | 9.337299453 | 126.8996408 | 1.95409E−29 | 5.35422E−27 |
| Stmn1 | −1.206782014 | 9.430175049 | 126.1409638 | 2.864E−29 | 7.71656E−27 |
| Yme1l1 | 0.579622353 | 9.30034005 | 126.1065549 | 2.91409E−29 | 7.72281E−27 |
| Tacc1 | 0.604302394 | 9.2685904 | 124.1370695 | 7.86206E−29 | 2.04997E−26 |
| Rps2 | −0.205226981 | 11.63566325 | 123.1141609 | 1.31652E−28 | 3.37823E−26 |
| Paip1 | 0.551270107 | 9.192203166 | 115.9410137 | 4.89646E−27 | 1.23681E−24 |
| Actr2 | 0.392668307 | 9.67646915 | 114.132176 | 1.21906E−26 | 3.03189E−24 |
| Picalm | 0.555431958 | 9.321258484 | 113.9378103 | 1.3446E−26 | 3.29346E−24 |
| Sbno1 | 0.513517128 | 9.426293354 | 113.1197864 | 2.03125E−26 | 4.90107E−24 |
| Inpp4a | 0.587876896 | 9.237580327 | 112.0650956 | 3.45769E−26 | 8.22015E−24 |
| Ldha | −0.320721971 | 10.35600944 | 111.3867557 | 4.86835E−26 | 1.12431E−23 |
| Sik1 | 0.602626506 | 9.263004564 | 111.4022104 | 4.83054E−26 | 1.12431E−23 |
| Wtap | 0.491265482 | 9.408000204 | 110.6189664 | 7.17102E−26 | 1.63277E−23 |
| Ets1 | 0.290857133 | 10.24994698 | 109.3408892 | 1.36644E−25 | 3.06804E−23 |
| Coro1a | −0.280380953 | 10.77303317 | 107.8067187 | 2.9631E−25 | 6.56185E−23 |
| Junb | 0.298950285 | 11.89123884 | 106.8133985 | 4.89122E−25 | 1.06853E−22 |
| Slc25a36 | 0.582838679 | 9.248175989 | 106.3278949 | 6.24906E−25 | 1.34696E−22 |
| Vps54 | 0.565971111 | 9.235814078 | 103.7126119 | 2.33898E−24 | 4.97525E−22 |
| Slc24a5 | 0.553964628 | 9.200019039 | 103.63554 | 2.43176E−24 | 5.10543E−22 |
| Tra2b | 0.370231771 | 9.814904189 | 103.315665 | 2.85785E−24 | 5.92307E−22 |
| Gm11713 | 0.572636965 | 9.228906171 | 103.2557973 | 2.94552E−24 | 6.02751E−22 |
| Ms4a4b | −0.307441662 | 10.75184171 | 100.524946 | 1.16915E−23 | 2.36256E−21 |
| Ubc | −0.296729819 | 11.05003167 | 100.4017801 | 1.24416E−23 | 2.4831E−21 |
| Gapdh | −0.27320874 | 10.56202545 | 99.15190914 | 2.33857E−23 | 4.61041E−21 |
| Atp5b | −0.290696215 | 10.21322353 | 98.82442542 | 2.7591E−23 | 5.37394E−21 |
| Clk1 | 0.36378697 | 9.76646424 | 96.66015417 | 8.23092E−23 | 1.58406E−20 |
| Rbm39 | 0.215576064 | 10.68049787 | 92.55576108 | 6.54543E−22 | 1.24486E−19 |
| Tuba1b | −0.552571308 | 9.729974254 | 92.05832174 | 8.41593E−22 | 1.582E−19 |
| Ass1 | −0.523431526 | 9.534289055 | 91.07958592 | 1.38008E−21 | 2.56441E−19 |
| Gm28875 | 0.563226861 | 9.244792702 | 90.68509289 | 1.68458E−21 | 3.09464E−19 |
| Ran | −0.404512958 | 9.801116978 | 89.98963 | 2.39412E−21 | 4.34868E−19 |
| Mxd1 | 0.445030151 | 9.600631761 | 88.53118238 | 5.00398E−21 | 8.98826E−19 |
| Cdc42 | −0.241615643 | 10.40474043 | 88.09235834 | 6.24685E−21 | 1.10974E−18 |
| Dctn4 | 0.515634719 | 9.226791755 | 87.91551187 | 6.83109E−21 | 1.20034E−18 |
| Tagln2 | −0.315292626 | 10.29861125 | 85.67548392 | 2.12025E−20 | 3.68559E−18 |
| Pycard | −0.480518193 | 9.577079204 | 85.3625029 | 2.48386E−20 | 4.27172E−18 |
| Iqgap1 | 0.367736638 | 9.634427689 | 85.30814659 | 2.55309E−20 | 4.34456E−18 |
| Ccna2 | −0.429260011 | 9.193741049 | 85.24349744 | 2.63795E−20 | 4.44219E−18 |
| Rps17 | −0.238111557 | 10.6149625 | 84.2556873 | 4.34751E−20 | 7.24555E−18 |
| Tspo | −0.3241737 | 9.918108481 | 82.82928905 | 8.94546E−20 | 1.47564E−17 |
| Birc5 | −0.774306859 | 9.251476471 | 82.78125795 | 9.16549E−20 | 1.49666E−17 |
| Rps20 | −0.199278964 | 11.06854686 | 82.45020041 | 1.08366E−19 | 1.75185E−17 |
| Lsp1 | −0.289441501 | 10.32146402 | 82.23662981 | 1.20732E−19 | 1.93242E−17 |
| Tmsb4x | −0.124205111 | 14.24920305 | 81.97789106 | 1.37618E−19 | 2.1811E−17 |
| Anxa2 | −0.424357411 | 9.842409891 | 81.44249127 | 1.80436E−19 | 2.83198E−17 |
| Slc25a5 | −0.256183417 | 10.39387945 | 80.44715179 | 2.98585E−19 | 4.64127E−17 |
| Twistnb | 0.466840475 | 9.374854863 | 80.3380422 | 3.15535E−19 | 4.85804E−17 |
| Vmp1 | 0.412669378 | 9.490475476 | 79.90893633 | 3.92069E−19 | 5.97943E−17 |
| Erh | −0.369077248 | 9.741195422 | 79.50363006 | 4.8134E−19 | 7.27228E−17 |
| Luc7l2 | 0.340413015 | 9.66558153 | 78.75848933 | 7.01865E−19 | 1.05059E−16 |
| Hmgb2 | −0.305301671 | 10.71128878 | 77.75424372 | 1.16692E−18 | 1.73069E−16 |
| 2310035C23Rik | 0.479288441 | 9.250856289 | 77.25219276 | 1.50464E−18 | 2.21128E−16 |
| D17H6S53E | 0.406173804 | 9.17696648 | 76.49933424 | 2.20287E−18 | 3.20826E−16 |
| Fhl3 | 0.398167305 | 9.169266684 | 76.3836551 | 2.33577E−18 | 3.37143E−16 |
| Rpl10a | −0.169361349 | 11.14217591 | 73.76932407 | 8.78005E−18 | 1.25609E−15 |
| Ube2c | −0.641235053 | 9.233348605 | 72.70415792 | 1.50616E−17 | 2.13585E−15 |
| Rpl32 | −0.117506557 | 12.44734553 | 72.55274494 | 1.62627E−17 | 2.28611E−15 |
| Rpl7 | −0.15134652 | 11.21186942 | 71.29300761 | 3.07925E−17 | 4.26112E−15 |
| Rpl28 | −0.19927858 | 10.72551078 | 71.29000083 | 3.08395E−17 | 4.26112E−15 |
| 4933434E20Rik | 0.427011884 | 9.335042118 | 71.15520058 | 3.30201E−17 | 4.52376E−15 |
| Myef2 | 0.473342591 | 9.220160509 | 70.98360765 | 3.60205E−17 | 4.89334E−15 |
| Asf1b | −0.519244424 | 9.223192204 | 69.8277614 | 6.47156E−17 | 8.71827E−15 |
| Hsf2 | 0.362266417 | 9.168337049 | 69.71039283 | 6.86833E−17 | 9.17631E−15 |
| Arhgdib | −0.215029523 | 10.73176838 | 69.28228906 | 8.53324E−17 | 1.13072E−14 |
| Ube2h | 0.460203792 | 9.263527957 | 68.76398418 | 1.10981E−16 | 1.45863E−14 |
| Gsk3b | 0.407848736 | 9.383828957 | 68.63067832 | 1.18742E−16 | 1.54806E−14 |
| Itgb1 | −0.651985291 | 9.299264118 | 68.61024305 | 1.19979E−16 | 1.55167E−14 |
| Rps27a | −0.126520352 | 11.80972706 | 68.55707054 | 1.23258E−16 | 1.58142E−14 |
| Brd4 | 0.414924172 | 9.358976296 | 68.13900406 | 1.52367E−16 | 1.9395E−14 |
| Pkm | −0.263117525 | 10.16193974 | 67.81711708 | 1.79385E−16 | 2.26558E−14 |
| Eif4a1 | −0.22402961 | 10.43971408 | 67.73133076 | 1.87363E−16 | 2.34799E−14 |
| Rpl10 | −0.154085529 | 11.48655718 | 67.63680055 | 1.96564E−16 | 2.44435E−14 |
| Cdca3 | −0.407079178 | 9.196715741 | 67.36575915 | 2.25532E−16 | 2.78317E−14 |
| Rps8 | −0.141029567 | 11.59257343 | 67.11812291 | 2.55717E−16 | 3.13176E−14 |
| Cd48 | −0.386951826 | 9.721176096 | 66.9921926 | 2.72585E−16 | 3.31323E−14 |
| Paxbp1 | 0.466529975 | 9.251251081 | 66.57229938 | 3.37293E−16 | 4.06916E−14 |
| Rps3 | −0.111776312 | 11.96705488 | 66.29601134 | 3.88043E−16 | 4.64674E−14 |
| Otub1 | 0.41672661 | 9.332259923 | 66.2658541 | 3.94026E−16 | 4.68369E−14 |
| Myl6 | −0.177794839 | 10.93119837 | 65.70031043 | 5.24976E−16 | 6.19472E−14 |
| Rnf157 | 0.433755297 | 9.351493602 | 64.59611437 | 9.19365E−16 | 1.07699E−13 |
| Rps10 | −0.147538768 | 11.22179603 | 64.31354938 | 1.06113E−15 | 1.23412E−13 |
| Eef1a1 | −0.110321731 | 12.38176503 | 63.52961517 | 1.57974E−15 | 1.82415E−13 |
| Rps12 | −0.195856238 | 10.77060475 | 63.26015038 | 1.81132E−15 | 2.07672E−13 |
| Gnb2l1 | −0.190267377 | 10.54006448 | 62.23026714 | 3.05555E−15 | 3.4786E−13 |
| Nusap1 | −0.491535516 | 9.207147356 | 61.59057605 | 4.22838E−15 | 4.78013E−13 |
| Il18r1 | 0.368848685 | 9.526791354 | 61.355196 | 4.76532E−15 | 5.34973E−13 |
| Gzmk | −0.690300473 | 9.363676844 | 61.27842647 | 4.9548E−15 | 5.52409E−13 |
| Tyms | −0.498994778 | 9.238838131 | 61.20745879 | 5.13666E−15 | 5.66822E−13 |
| Lrrc8a | 0.409765583 | 9.190599691 | 61.20074377 | 5.15421E−15 | 5.66822E−13 |
| Cdca8 | −0.458035324 | 9.207869503 | 60.94749215 | 5.86176E−15 | 6.40278E−13 |
| Rrm2 | −0.611980984 | 9.247497636 | 60.56190851 | 7.13004E−15 | 7.73586E−13 |
| Ccnb2 | −0.485513492 | 9.212240288 | 59.84990711 | 1.02374E−14 | 1.10332E−12 |
| Rpl31 | −0.170290124 | 10.87151483 | 59.78576465 | 1.05766E−14 | 1.13232E−12 |
| Pnrc1 | 0.208894663 | 10.57788165 | 59.46508287 | 1.24483E−14 | 1.32395E−12 |
| Clic1 | −0.227413644 | 10.32554613 | 59.07912283 | 1.51458E−14 | 1.6003E−12 |
| Ccnb1 | −0.297805543 | 9.183009169 | 58.84515862 | 1.70581E−14 | 1.79066E−12 |
| Gnl3l | 0.393730748 | 9.189382723 | 58.79316531 | 1.75149E−14 | 1.82674E−12 |
| Zscan26 | 0.396044821 | 9.188162719 | 58.13789649 | 2.44373E−14 | 2.53239E−12 |
| B2m | −0.093766772 | 12.79982 | 57.0257555 | 4.30142E−14 | 4.42909E−12 |
| Gm26699 | 0.375519253 | 9.394464865 | 56.90560992 | 4.5724E−14 | 4.67831E−12 |
| Rplp0 | −0.108717289 | 12.38775842 | 56.85972585 | 4.68034E−14 | 4.75864E−12 |
| Rpsa | −0.129421707 | 11.60009635 | 56.83524585 | 4.73896E−14 | 4.78813E−12 |
| Plcxd2 | 0.426020085 | 9.360396846 | 56.77268616 | 4.89214E−14 | 4.9122E−12 |
| Wnk1 | 0.324700375 | 9.627959621 | 56.58347879 | 5.38625E−14 | 5.37494E−12 |
| Foxo3 | 0.45328937 | 9.215455773 | 56.21309552 | 6.5027E−14 | 6.44924E−12 |
| Fbxo46 | 0.342072366 | 9.178401822 | 56.16217377 | 6.67331E−14 | 6.5781E−12 |
| Abcg1 | 0.404303623 | 9.204739969 | 56.05560672 | 7.045E−14 | 6.90239E−12 |
| Cks1b | −0.545261815 | 9.261603647 | 55.41157135 | 9.77593E−14 | 9.52034E−12 |
| Eomes | −0.641532838 | 9.215520909 | 55.26385568 | 1.05389E−13 | 1.02019E−11 |
| Ddx3y | 0.488648104 | 9.286792787 | 55.16274371 | 1.10952E−13 | 1.06765E−11 |
| Lbh | 0.382006384 | 9.408476281 | 54.62758716 | 1.45676E−13 | 1.39349E−11 |
| Ppia | −0.134187803 | 11.56530329 | 54.30730708 | 1.71463E−13 | 1.62098E−11 |
| Cdkn1a | 0.406730252 | 9.693181563 | 54.31309281 | 1.70959E−13 | 1.62098E−11 |
| Rpl7a | −0.167038867 | 10.71263743 | 53.61755631 | 2.43573E−13 | 2.2893E−11 |
| Gngt2 | 0.409025485 | 9.363461133 | 52.9479409 | 3.42507E−13 | 3.20056E−11 |
| Rps7 | −0.11087119 | 11.94311471 | 52.54889015 | 4.1967E−13 | 3.89907E−11 |
| Itgb7 | −0.270792151 | 9.956558259 | 52.49357441 | 4.31658E−13 | 3.98754E−11 |
| Neurl3 | 0.323822382 | 9.619672539 | 52.41712233 | 4.48794E−13 | 4.12227E−11 |
| Rplp1 | −0.124745324 | 11.7032513 | 52.07151016 | 5.35158E−13 | 4.88777E−11 |
| Mcmbp | 0.413813179 | 9.263665081 | 51.20857013 | 8.30552E−13 | 7.5431E−11 |
| Itpkb | 0.339679714 | 9.50966549 | 51.02768436 | 9.10724E−13 | 8.22501E−11 |
| Rpl23 | −0.117810619 | 11.70858916 | 50.8216993 | 1.01149E−12 | 9.08433E−11 |
| Glipr2 | −0.378200114 | 9.484106509 | 50.74315683 | 1.05279E−12 | 9.40298E−11 |
| Rplp2 | −0.114755509 | 11.91005984 | 50.19613435 | 1.39122E−12 | 1.23574E−10 |
| Ppib | −0.241497971 | 9.880914383 | 49.93316246 | 1.59073E−12 | 1.40523E−10 |
| Rbbp7 | −0.389687456 | 9.3983099 | 49.65461608 | 1.83338E−12 | 1.61079E−10 |
| Etv3 | 0.407752576 | 9.25329147 | 49.62740311 | 1.85899E−12 | 1.62446E−10 |
| Ptma | −0.16554087 | 11.28820186 | 49.53884871 | 1.94482E−12 | 1.69032E−10 |
| Hmgb1 | −0.226258893 | 10.2970404 | 49.01334479 | 2.54227E−12 | 2.19777E−10 |
| Arhgap31 | 0.330556748 | 9.65435635 | 48.91411752 | 2.67419E−12 | 2.29952E−10 |
| Gigyf1 | 0.347864515 | 9.189703078 | 48.79333282 | 2.84405E−12 | 2.41067E−10 |
| Txn1 | −0.239849684 | 10.00206855 | 48.790484 | 2.84818E−12 | 2.41067E−10 |
| Rasgrp2 | −0.418570333 | 9.377071127 | 48.79152979 | 2.84667E−12 | 2.41067E−10 |
| Clasp2 | 0.332052036 | 9.17236319 | 48.4325801 | 3.41841E−12 | 2.86332E−10 |
| Rps3a1 | −0.108391678 | 11.67388738 | 48.43755822 | 3.40975E−12 | 2.86332E−10 |
| Hnrnpk | −0.203363418 | 10.15623829 | 48.37079186 | 3.52784E−12 | 2.93974E−10 |
| Dut | −0.4252743 | 9.370293948 | 47.66015211 | 5.06891E−12 | 4.20225E−10 |
| 1500009L16Rik | −0.436094888 | 9.246962693 | 47.54794259 | 5.36751E−12 | 4.4271E−10 |
| Rps23 | −0.091493016 | 12.48529685 | 47.41265727 | 5.75101E−12 | 4.71933E−10 |
| Rpl5 | −0.166750551 | 10.56753913 | 47.10915065 | 6.71415E−12 | 5.48187E−10 |
| Cdc45 | −0.176040165 | 9.168332637 | 46.94437406 | 7.30303E−12 | 5.9327E−10 |
| Psma4 | −0.320075079 | 9.543453421 | 46.8948269 | 7.49002E−12 | 6.05418E−10 |
| Dusp5 | 0.244343465 | 9.994465583 | 46.81799425 | 7.78949E−12 | 6.26492E−10 |
| Tgif1 | 0.288197973 | 9.720285893 | 46.55851466 | 8.8923E−12 | 7.11648E−10 |
| Iqsec1 | 0.306868317 | 9.175097412 | 46.31161233 | 1.00864E−11 | 8.03238E−10 |
| Arhgap26 | 0.362226979 | 9.20131511 | 46.26928545 | 1.03067E−11 | 8.16756E−10 |
| Smc2 | −0.457838158 | 9.240576863 | 46.18600984 | 1.07542E−11 | 8.48062E−10 |
| Aurkb | −0.227897739 | 9.176287527 | 46.17329213 | 1.08243E−11 | 8.49441E−10 |
| Themis | 0.338780405 | 9.406121809 | 46.15809092 | 1.09086E−11 | 8.51922E−10 |
| Rad51ap1 | −0.168933582 | 9.168873929 | 45.83353646 | 1.28741E−11 | 1.00059E−09 |
| Ddit4 | −0.457302383 | 9.366175801 | 45.70124776 | 1.37735E−11 | 1.06285E−09 |
| Set | 0.268072579 | 9.77371175 | 45.6965463 | 1.38066E−11 | 1.06285E−09 |
| Bcl2l1 | 0.374282608 | 9.376443509 | 45.58015518 | 1.46519E−11 | 1.12257E−09 |
| Tpx2 | −0.255816003 | 9.180936262 | 45.48370732 | 1.53914E−11 | 1.17367E−09 |
| Uvssa | 0.357771198 | 9.191219939 | 45.45775589 | 1.55967E−11 | 1.18374E−09 |
| Uhrf2 | 0.345073862 | 9.554452999 | 45.43283888 | 1.57964E−11 | 1.19329E−09 |
| Rbm3 | −0.174412186 | 10.73098646 | 45.27873534 | 1.70895E−11 | 1.28497E−09 |
| Rap2b | 0.386872593 | 9.217861261 | 45.25646564 | 1.72849E−11 | 1.29365E−09 |
| Ahnak | 0.279946145 | 9.648666059 | 45.20386587 | 1.77554E−11 | 1.32274E−09 |
| Anp32a | −0.238121521 | 9.878253526 | 44.91218759 | 2.06071E−11 | 1.52814E−09 |
| Anxa6 | −0.266357286 | 9.747058157 | 44.88962553 | 2.08459E−11 | 1.53879E−09 |
| Hspa5 | −0.188078636 | 10.97852379 | 44.34155639 | 2.75799E−11 | 2.02662E−09 |
| Calr | −0.237935659 | 10.02077588 | 44.24976673 | 2.89039E−11 | 2.1143E−09 |
| Tk1 | −0.276494249 | 9.186384692 | 44.05815286 | 3.18763E−11 | 2.32123E−09 |
| Ezr | −0.28511791 | 9.722755344 | 43.93118787 | 3.40126E−11 | 2.46568E−09 |
| Peli1 | 0.262373761 | 9.756230653 | 43.77706211 | 3.67993E−11 | 2.65579E−09 |
| Hmmr | −0.151166002 | 9.167541924 | 43.57211811 | 4.0862E−11 | 2.93589E−09 |
| Dbi | −0.354303671 | 9.462114256 | 43.44556351 | 4.35919E−11 | 3.11817E−09 |
| Hint1 | −0.205762868 | 10.08266688 | 43.31598681 | 4.65763E−11 | 3.31697E−09 |
| Snrpe | −0.239207361 | 9.827296541 | 43.27064695 | 4.76681E−11 | 3.37984E−09 |
| Tppp3 | −0.326363173 | 9.18599552 | 43.02109733 | 5.41527E−11 | 3.82285E−09 |
| Cct8 | −0.301160499 | 9.521391609 | 42.40689585 | 7.41282E−11 | 5.21025E−09 |
| Hmgcr | 0.36841636 | 9.25124838 | 42.32625686 | 7.72485E−11 | 5.40606E−09 |
| Ly6c2 | −0.697969266 | 9.265996044 | 42.30561042 | 7.80683E−11 | 5.43988E−09 |
| Tnfaip3 | 0.227027416 | 10.12507605 | 42.22671488 | 8.12821E−11 | 5.61541E−09 |
| Mki67 | −0.3515189 | 9.202982177 | 42.22810773 | 8.12242E−11 | 5.61541E−09 |
| Rrm1 | −0.384070718 | 9.219634699 | 41.86633532 | 9.77309E−11 | 6.72305E−09 |
| Hist1h2ap | −0.286036608 | 9.18659925 | 41.65572091 | 1.08846E−10 | 7.45598E−09 |
| Stim2 | 0.362672122 | 9.238378298 | 41.47872855 | 1.1916E−10 | 8.12805E−09 |
| Fn3krp | 0.184846223 | 9.161101118 | 41.29537404 | 1.30878E−10 | 8.88982E−09 |
| Bmp2k | 0.358909864 | 9.218888635 | 41.24306732 | 1.34428E−10 | 9.05483E−09 |
| Rps24 | −0.101666272 | 12.00584544 | 41.25017653 | 1.3394E−10 | 9.05483E−09 |
| Spc24 | −0.346692321 | 9.204100128 | 40.95893737 | 1.55461E−10 | 1.04282E−08 |
| Hnrnpa2b1 | −0.196534148 | 10.12046099 | 40.88778863 | 1.61225E−10 | 1.07274E−08 |
| S1pr1 | −0.422059619 | 9.290338593 | 40.88748874 | 1.6125E−10 | 1.07274E−08 |
| mt-Atp6 | 0.102698372 | 12.47134724 | 40.86302484 | 1.63281E−10 | 1.08181E−08 |
| Prdx1 | −0.232856001 | 9.945082204 | 40.62557883 | 1.84377E−10 | 1.21659E−08 |
| Xist | −0.490990751 | 9.888398691 | 40.56781288 | 1.89909E−10 | 1.248E−08 |
| Smap2 | 0.28963393 | 9.515102178 | 40.50424865 | 1.96189E−10 | 1.28404E−08 |
| Rpl26 | −0.103119108 | 11.57423238 | 40.39614168 | 2.0735E−10 | 1.35162E−08 |
| Btf3 | −0.134387238 | 10.715474 | 40.35161787 | 2.1213E−10 | 1.37722E−08 |
| Rps4x | −0.09543889 | 11.84794084 | 40.24202475 | 2.24369E−10 | 1.45086E−08 |
| Naca | −0.135088853 | 10.80132827 | 40.22731503 | 2.26065E−10 | 1.456E−08 |
| Zbtb21 | 0.346325707 | 9.20850205 | 40.1126819 | 2.39728E−10 | 1.53787E−08 |
| Ppp1ca | −0.180999333 | 10.29795938 | 40.09980116 | 2.41314E−10 | 1.54193E−08 |
| Anp32e | −0.31717177 | 9.516881137 | 39.89016764 | 2.68652E−10 | 1.70985E−08 |
| Cct4 | −0.269302589 | 9.5936703 | 39.78464151 | 2.83566E−10 | 1.7977E−08 |
| Yes1 | 0.387710552 | 9.221085842 | 39.58488074 | 3.14106E−10 | 1.98353E−08 |
| Reep5 | −0.27118009 | 9.637135956 | 39.51909329 | 3.24867E−10 | 2.04351E−08 |
| Sept2 | 0.342873186 | 9.274290012 | 39.48946663 | 3.29834E−10 | 2.0667E−08 |
| Rpl35a | −0.09831683 | 11.7234212 | 39.28221717 | 3.66766E−10 | 2.28925E−08 |
| Cotl1 | −0.182341504 | 10.46112717 | 39.11250618 | 4.00073E−10 | 2.48753E−08 |
| Il7r | 0.272892916 | 9.753485896 | 38.72699605 | 4.87421E−10 | 3.01902E−08 |
| Psmb7 | −0.295620167 | 9.46523868 | 38.2618874 | 6.18591E−10 | 3.81685E−08 |
| Atf7ip | 0.365085149 | 9.253987334 | 37.9190664 | 7.3741E−10 | 4.53269E−08 |
| Hnrnpul1 | 0.306269675 | 9.37910581 | 37.75916648 | 8.00397E−10 | 4.90122E−08 |
| Cdkn3 | −0.220464584 | 9.177347936 | 37.69331595 | 8.27877E−10 | 5.05037E−08 |
| Dnajb1 | 0.393957367 | 10.31781379 | 37.62379926 | 8.57913E−10 | 5.21392E−08 |
| Cd2 | −0.195141092 | 10.31973533 | 37.56156193 | 8.85728E−10 | 5.3628E−08 |
| Klhl9 | 0.321655077 | 9.198908172 | 37.50704817 | 9.10833E−10 | 5.49422E−08 |
| Cenpm | −0.21208704 | 9.175769069 | 37.44666956 | 9.3947E−10 | 5.6459E−08 |
| Mcm2 | −0.372335465 | 9.227695356 | 37.4049719 | 9.59771E−10 | 5.74654E−08 |

TABLE 3B
LPL-1 vs. All

| GeneSymbol | logFC | logCPM | LR | PValue | FDR |
|---|---|---|---|---|---|
| Actb | −1.788578756 | 13.40456535 | 1969.985989 | 0 | 0 |
| Vps37b | 0.895007394 | 10.60462528 | 1103.972392 | 4.5239E−242 | 3.6566E−238 |
| S100a4 | −2.066980708 | 9.866840932 | 990.3446629 | 2.2542E−217 | 1.2147E−213 |
| S100a6 | −1.154115196 | 10.99649875 | 760.3528646 | 2.2508E−167 | 9.0966E−164 |
| Crem | 1.135777664 | 10.06722635 | 752.2332434 | 1.3116E−165 | 4.2408E−162 |
| Ifngr1 | 0.612024932 | 11.11302914 | 721.8445664 | 5.3157E−159 | 1.4322E−155 |
| Pfn1 | −0.538711172 | 12.14558847 | 686.3702201 | 2.7519E−151 | 6.3553E−148 |
| Plac8 | 1.128214216 | 10.5691871 | 665.727518 | 8.4869E−147 | 1.715E−143 |
| Klf2 | −1.940850631 | 9.960578273 | 662.9676093 | 3.3803E−146 | 6.0718E−143 |
| Rgs2 | 1.097780206 | 10.06000559 | 658.8370261 | 2.6746E−145 | 4.3237E−142 |
| Got1 | 1.084074843 | 9.855972072 | 655.3423412 | 1.5391E−144 | 2.2619E−141 |
| Il21r | 0.809669496 | 10.19619539 | 636.1687863 | 2.276E−140 | 3.0662E−137 |
| Coro1a | −0.610607496 | 10.77323885 | 589.3688971 | 3.437E−130 | 4.2741E−127 |
| Serpinb9 | 1.081184718 | 9.858313142 | 578.5602139 | 7.7137E−128 | 8.9071E−125 |
| Eif1 | 0.337833647 | 11.85889679 | 560.5165898 | 6.49E−124 | 6.9945E−121 |
| Lgals1 | −0.72773285 | 11.62802019 | 535.1379291 | 2.1536E−118 | 2.176E−115 |
| Lsp1 | −0.654984065 | 10.32147718 | 488.4511929 | 3.0961E−108 | 2.9442E−105 |
| Ptprcap | −0.783000793 | 10.0401488 | 488.2382418 | 3.4447E−108 | 3.0937E−105 |
| Pycard | −1.085203588 | 9.577184042 | 487.7836331 | 4.3258E−108 | 3.6806E−105 |
| Dgat1 | 0.89679146 | 9.778871165 | 483.8844962 | 3.0514E−107 | 2.4664E−104 |
| Fosl2 | 0.952434136 | 9.53162468 | 477.9445926 | 5.9841E−106 | 4.6066E−103 |
| Rps27rt | 0.520913608 | 11.16205822 | 470.2190332 | 2.8715E−104 | 2.11E−101 |
| Neurl3 | 0.799145197 | 9.619598144 | 457.07289 | 2.0839E−101 | 1.46474E−98 |
| Rps27 | 0.285776264 | 13.23482323 | 418.9475401 | 4.13549E−93 | 2.7856E−90 |
| Fth1 | 0.376259293 | 11.58476825 | 415.2458293 | 2.644E−92 | 1.64396E−89 |
| Tmsb4x | −0.273689693 | 14.24925717 | 415.3072706 | 2.56382E−92 | 1.64396E−89 |
| H2-K1 | 0.343352537 | 11.6100296 | 411.2718478 | 1.93767E−91 | 1.16016E−88 |
| KIrc1 | 1.023930603 | 9.658425729 | 411.0322548 | 2.1849E−91 | 1.26147E−88 |
| Ubald2 | 0.549709616 | 10.62002464 | 403.3569592 | 1.0237E−89 | 5.70662E−87 |
| Cd52 | −0.444841022 | 11.79358725 | 396.9799802 | 2.50243E−88 | 1.34847E−85 |
| Cfl1 | −0.392116577 | 11.11291947 | 384.8948211 | 1.06979E−85 | 5.57879E−83 |
| Il4ra | 0.774913613 | 9.499541933 | 367.1576528 | 7.78158E−82 | 3.93116E−79 |
| Dusp5 | 0.581228407 | 9.994361734 | 364.1948693 | 3.43699E−81 | 1.68371E−78 |
| Samsn1 | 0.721144616 | 9.795511159 | 359.8134916 | 3.09169E−80 | 1.47001E−77 |
| Fgl2 | 0.863824189 | 9.496139473 | 356.3691619 | 1.7386E−79 | 8.03034E−77 |
| Rps21 | 0.368294596 | 10.92388869 | 355.6060267 | 2.54903E−79 | 1.14466E−76 |
| Arhgdib | −0.446836849 | 10.7318272 | 353.5929365 | 6.9942E−79 | 3.0559E−76 |
| H2-D1 | 0.251752892 | 12.28194438 | 353.1089558 | 8.91515E−79 | 3.79269E−76 |
| Gna13 | 0.565688846 | 9.98513228 | 340.9752456 | 3.91273E−76 | 1.62188E−73 |
| Ddx5 | 0.344568543 | 11.1991571 | 340.3406904 | 5.37866E−76 | 2.17379E−73 |
| Cnn2 | −0.654811498 | 9.902885218 | 336.4034115 | 3.87396E−75 | 1.52748E−72 |
| Emp3 | −0.70120027 | 9.864713566 | 333.8561537 | 1.38972E−74 | 5.34909E−72 |
| Cd48 | −0.798426594 | 9.72124298 | 328.3159143 | 2.23659E−73 | 8.40854E−71 |
| Pik3r1 | 0.73885528 | 9.484799174 | 319.99719 | 1.45049E−71 | 5.32921E−69 |
| Lilrb4a | 0.738717163 | 9.646925283 | 318.7921692 | 2.65457E−71 | 9.5364E−69 |
| Ndufa4 | −0.679488465 | 9.73553315 | 315.5972178 | 1.31808E−70 | 4.6322E−68 |
| mt-Co3 | −0.28661259 | 12.09929008 | 308.8719528 | 3.8456E−69 | 1.32272E−66 |
| Prr7 | 0.638751806 | 9.824437298 | 306.931633 | 1.01779E−68 | 3.42783E−66 |
| Fasl | 0.69087468 | 9.594176945 | 306.2337496 | 1.44442E−68 | 4.7654E−66 |
| Slc25a5 | −0.452500278 | 10.39410252 | 304.0526899 | 4.31366E−68 | 1.39469E−65 |
| Rac2 | −0.337772566 | 11.09602488 | 296.8583091 | 1.59313E−66 | 5.04992E−64 |
| Dnaja1 | 0.566084182 | 10.76357045 | 294.7162074 | 4.66622E−66 | 1.45066E−63 |
| Nrip1 | 0.732165744 | 9.582519625 | 288.2422587 | 1.20104E−64 | 3.59555E−62 |
| Arpc3 | 0.372459303 | 10.54021408 | 288.2457578 | 1.19893E−64 | 3.59555E−62 |
| Litaf | 0.809822856 | 9.445009121 | 284.4411901 | 8.08743E−64 | 2.37712E−61 |
| Arpc1b | −0.375752302 | 10.55364549 | 281.7754455 | 3.08106E−63 | 8.89436E−61 |
| Tubb5 | −0.772688378 | 10.00334547 | 281.1572307 | 4.20163E−63 | 1.19164E−60 |
| Ctla2a | 0.700790544 | 10.33575323 | 279.3695207 | 1.03036E−62 | 2.87186E−60 |
| Rpl37a | 0.210248664 | 12.61564007 | 277.6971692 | 2.38468E−62 | 6.53402E−60 |
| Bin2 | −0.728028996 | 9.612828291 | 277.1285376 | 3.17211E−62 | 8.54673E−60 |
| Wdr89 | 0.513393949 | 10.42643281 | 274.3649881 | 1.26942E−61 | 3.36418E−59 |
| Eif5 | 0.511485385 | 9.823441033 | 269.7699212 | 1.27366E−60 | 3.32097E−58 |
| Nabp1 | 0.636082496 | 9.632410631 | 268.2210034 | 2.77101E−60 | 7.11051E−58 |
| Klrk1 | 0.934839579 | 9.564414696 | 268.1106967 | 2.92873E−60 | 7.3978E−58 |
| Rplp0 | −0.218100349 | 12.38791042 | 267.9073344 | 3.24342E−60 | 8.06663E−58 |
| Rcbtb2 | 0.809053481 | 9.39894406 | 265.5508845 | 1.05829E−59 | 2.59216E−57 |
| Rpl38 | 0.237996532 | 11.67460313 | 258.5201237 | 3.60659E−58 | 8.70212E−56 |
| Tmem176a | −1.165497721 | 9.404480668 | 252.8672547 | 6.15696E−57 | 1.46373E−54 |
| mt-Atp6 | −0.243334052 | 12.4711756 | 248.1279894 | 6.64601E−56 | 1.55709E−53 |
| Capzb | −0.431404911 | 10.15899075 | 247.9573988 | 7.24023E−56 | 1.67208E−53 |
| Clic1 | −0.422522733 | 10.32564272 | 246.7932166 | 1.29887E−55 | 2.95739E−53 |
| Ifrd1 | 0.521730657 | 10.02583298 | 238.8278921 | 7.08402E−54 | 1.59056E−51 |
| Nfkbia | −0.581770075 | 10.83335684 | 237.7851151 | 1.1958E−53 | 2.64812E−51 |
| Usmg5 | 0.41505392 | 10.06859317 | 237.6977148 | 1.24944E−53 | 2.72953E−51 |
| Nr3c1 | 0.688319036 | 9.448726347 | 237.0161873 | 1.75925E−53 | 3.792E−51 |
| Actr3 | −0.415162569 | 10.23427966 | 234.8065523 | 5.33531E−53 | 1.13488E−50 |
| Ppp1ca | −0.397132915 | 10.29810957 | 233.8723064 | 8.52878E−53 | 1.7906E−50 |
| Cnbp | 0.356953072 | 10.25780671 | 233.8107991 | 8.7963E−53 | 1.82309E−50 |
| Gimap6 | 0.391836768 | 10.4658122 | 231.7934375 | 2.42231E−52 | 4.95685E−50 |
| Gnai2 | −0.456079708 | 10.17424924 | 231.4370798 | 2.89697E−52 | 5.85405E−50 |
| Pglyrp1 | −0.932489522 | 9.331204668 | 231.4010017 | 2.94993E−52 | 5.88747E−50 |
| Rplp2 | 0.21411519 | 11.91020827 | 230.6878952 | 4.22012E−52 | 8.31982E−50 |
| Lilr4b | 0.578605702 | 9.77812791 | 228.9456823 | 1.01222E−51 | 1.97151E−49 |
| Irak2 | 0.726366279 | 9.278620156 | 226.4881484 | 3.47731E−51 | 6.69217E−49 |
| Rpl35 | −0.293445674 | 11.18862876 | 225.0845461 | 7.03672E−51 | 1.3383E−48 |
| Lmna | 0.838878501 | 9.713616963 | 223.8901563 | 1.28196E−50 | 2.40979E−48 |
| Cbx3 | −0.793973529 | 9.51797351 | 221.1859312 | 4.98546E−50 | 9.26378E−48 |
| Itk | 0.497130976 | 9.814380172 | 220.0345571 | 8.88885E−50 | 1.63292E−47 |
| Jun | −1.278105538 | 9.603448288 | 218.9495885 | 1.53288E−49 | 2.78433E−47 |
| Ifitm2 | 0.728838581 | 10.68450898 | 218.2894633 | 2.13551E−49 | 3.83585E−47 |
| Nr4a2 | 0.715704419 | 9.52121308 | 217.1365889 | 3.81052E−49 | 6.76933E−47 |
| P2ry10 | 0.557735676 | 9.588148915 | 216.0497309 | 6.57768E−49 | 1.15581E−46 |
| Dennd4a | 0.661822792 | 9.353169932 | 215.1945639 | 1.0107E−48 | 1.75688E−46 |
| Dnajb6 | 0.402259307 | 10.0235756 | 214.4131266 | 1.49656E−48 | 2.57376E−46 |
| Rps24 | 0.201933056 | 12.00595021 | 212.9332807 | 3.14722E−48 | 5.35558E−46 |
| Il12rb2 | 0.713180429 | 9.345960823 | 210.587897 | 1.02236E−47 | 1.72161E−45 |
| Fus | 0.415736893 | 9.89924265 | 209.6682075 | 1.62276E−47 | 2.70449E−45 |
| Actg1 | −0.305072557 | 12.09966478 | 209.2739452 | 1.97821E−47 | 3.26323E−45 |
| Cotl1 | −0.387624119 | 10.46139499 | 208.5595047 | 2.83234E−47 | 4.62502E−45 |
| Hilpda | 0.643315922 | 9.667586402 | 207.5609804 | 4.67741E−47 | 7.5615E−45 |
| Abracl | −0.459491705 | 9.935333895 | 204.9361765 | 1.74868E−46 | 2.79893E−44 |
| Stmn1 | −1.408402171 | 9.430733602 | 204.5829412 | 2.08827E−46 | 3.3097E−44 |
| S100a13 | −0.460207609 | 9.897903663 | 200.3466085 | 1.75467E−45 | 2.75397E−43 |
| Zyx | −0.78157278 | 9.311171714 | 198.7702945 | 3.87423E−45 | 6.02219E−43 |
| mt-Co1 | −0.219039635 | 12.21547146 | 198.0155703 | 5.66095E−45 | 8.71571E−43 |
| Tnfrsf1b | 0.594415005 | 9.452768175 | 197.5495441 | 7.15471E−45 | 1.09116E−42 |
| Cks1b | −0.902420001 | 9.26180276 | 197.452059 | 7.51393E−45 | 1.13524E−42 |
| Cdkn1a | 0.684828228 | 9.693082603 | 197.3870862 | 7.7633E−45 | 1.16205E−42 |
| Rpl36 | 0.191378672 | 11.87412411 | 196.3232522 | 1.325E−44 | 1.96514E−42 |
| Prdx6 | 0.387419462 | 10.19719876 | 195.9655521 | 1.58592E−44 | 2.33073E−42 |
| 2810417H13Rik | −1.348762706 | 9.337778098 | 195.5943825 | 1.91112E−44 | 2.78335E−42 |
| Pkp3 | −0.590268938 | 9.709686255 | 194.7192546 | 2.96677E−44 | 4.28222E−42 |
| H2-Q7 | 0.318794872 | 10.6210556 | 194.3215104 | 3.6232E−44 | 5.18343E−42 |
| Rps28 | 0.239174935 | 11.70755238 | 194.1805529 | 3.88917E−44 | 5.51512E−42 |
| Myl6 | −0.272160511 | 10.93133884 | 189.5951424 | 3.89676E−43 | 5.47782E−41 |
| Itgb7 | −0.467285899 | 9.956624675 | 189.2773925 | 4.57154E−43 | 6.37099E−41 |
| 1700097N02Rik | −0.721214639 | 9.227389704 | 187.3734805 | 1.19032E−42 | 1.64468E−40 |
| Prkca | 0.631509347 | 9.472730429 | 185.026078 | 3.87351E−42 | 5.30671E−40 |
| Abcb1b | 0.705256247 | 9.297095082 | 184.288226 | 5.61288E−42 | 7.62503E−40 |
| Bcl2l11 | 0.679389484 | 9.333331974 | 183.929455 | 6.72219E−42 | 9.05591E−40 |
| Slc3a2 | 0.401407628 | 10.08963433 | 183.7616752 | 7.31374E−42 | 9.7714E−40 |
| Osbpl9 | 0.664999167 | 9.306537304 | 183.4761973 | 8.44237E−42 | 1.10959E−39 |
| Lcp1 | −0.387042933 | 10.2911179 | 183.490149 | 8.38337E−42 | 1.10959E−39 |
| S1pr1 | −0.798212974 | 9.290347096 | 183.128455 | 1.0055E−41 | 1.31088E−39 |
| Sh2d1a | −0.668941826 | 9.507382786 | 182.7651867 | 1.20696E−41 | 1.56094E−39 |
| Rabac1 | 0.420182689 | 9.787494723 | 182.5076575 | 1.37378E−41 | 1.76258E−39 |
| Sec61b | −0.425196971 | 9.885092925 | 181.610947 | 2.15622E−41 | 2.74469E−39 |
| Riok1 | 0.638753261 | 9.379978763 | 181.4859042 | 2.29612E−41 | 2.89993E−39 |
| Gpr132 | 0.416559375 | 9.8660454 | 180.8336852 | 3.1871E−41 | 3.994E−39 |
| Crip1 | −0.493199874 | 11.66680468 | 180.0759095 | 4.66494E−41 | 5.80103E−39 |
| Rps16 | 0.16161083 | 12.35143085 | 179.5347494 | 6.12356E−41 | 7.55676E−39 |
| Abcb1a | 0.648799063 | 9.300398573 | 178.4612223 | 1.05052E−40 | 1.28657E−38 |
| BC021614 | −0.744896507 | 9.270925728 | 178.3219969 | 1.12669E−40 | 1.36948E−38 |
| Orai1 | 0.43534957 | 9.884592153 | 176.58257 | 2.70161E−40 | 3.25927E−38 |
| Gabpb1 | 0.596714389 | 9.478113973 | 175.9501956 | 3.71289E−40 | 4.44611E−38 |
| Rexo2 | −0.610677527 | 9.465244675 | 175.0647112 | 5.79529E−40 | 6.88873E−38 |
| Hspa8 | 0.298364729 | 12.33067999 | 171.6427177 | 3.23885E−39 | 3.82184E−37 |
| Tbc1d10c | −0.609447245 | 9.522567817 | 171.179049 | 4.08936E−39 | 4.79048E−37 |
| Ap2s1 | −0.560849494 | 9.498112489 | 171.1596432 | 4.12946E−39 | 4.80266E−37 |
| Atp5c1 | −0.393590419 | 9.901816954 | 170.3699703 | 6.14273E−39 | 7.0931E−37 |
| Chchd2 | 0.281156025 | 10.44216726 | 169.2568613 | 1.07517E−38 | 1.23271E−36 |
| Glipr2 | −0.62575301 | 9.484139571 | 169.1833807 | 1.11565E−38 | 1.27011E−36 |
| Ltb | −0.525135472 | 10.15487923 | 169.094768 | 1.16649E−38 | 1.31871E−36 |
| Apobec3 | −0.614418213 | 9.503277979 | 168.9346551 | 1.26431E−38 | 1.41936E−36 |
| Hsp90ab1 | 0.358235846 | 11.19703839 | 168.5749929 | 1.51499E−38 | 1.68906E−36 |
| Traf4 | 0.618977528 | 9.359798595 | 168.1736074 | 1.85388E−38 | 2.05273E−36 |
| Gch1 | 0.58977029 | 9.459478409 | 167.9384628 | 2.08662E−38 | 2.29471E−36 |
| Rps15a | 0.15887194 | 12.4038352 | 167.7357859 | 2.31053E−38 | 2.52379E−36 |
| Pim1 | −0.449599367 | 10.82518403 | 166.6702327 | 3.94877E−38 | 4.28429E−36 |
| AW112010 | −0.356352013 | 11.38310706 | 166.0238903 | 5.46572E−38 | 5.89059E−36 |
| Arpc5 | −0.374716295 | 10.09994182 | 165.8699709 | 5.90568E−38 | 6.32259E−36 |
| Tmem176b | −0.88387982 | 9.4480561 | 164.2381697 | 1.34194E−37 | 1.42722E−35 |
| Pde4b | 0.463371289 | 9.65862148 | 164.1390035 | 1.41057E−37 | 1.49041E−35 |
| Cdc42 | −0.290733616 | 10.40483847 | 164.0282804 | 1.49136E−37 | 1.56554E−35 |
| Rbm3 | −0.303875972 | 10.73119655 | 163.8078342 | 1.66626E−37 | 1.73785E−35 |
| Stat4 | 0.509660197 | 9.50654617 | 161.4842906 | 5.36238E−37 | 5.55694E−35 |
| Pomp | −0.461350255 | 9.70146416 | 160.7695414 | 7.68272E−37 | 7.91076E−35 |
| Isg15 | 0.724401352 | 9.495169432 | 160.4188089 | 9.16525E−37 | 9.37756E−35 |
| Tspo | −0.399054221 | 9.918101434 | 160.304487 | 9.70783E−37 | 9.87023E−35 |
| Arf4 | 0.373788321 | 9.974865872 | 159.6926899 | 1.32066E−36 | 1.33437E−34 |
| Gm11808 | 0.354253549 | 10.17414183 | 159.250362 | 1.64982E−36 | 1.65659E−34 |
| Flna | −0.685998565 | 9.27108819 | 159.1706353 | 1.71734E−36 | 1.71374E−34 |
| Uba52 | 0.326559163 | 10.2399743 | 158.987408 | 1.88318E−36 | 1.8677E−34 |
| Hsp90aa1 | 0.484135824 | 10.76861348 | 157.8713379 | 3.30183E−36 | 3.25472E−34 |
| Adora2a | 0.552025884 | 9.426699157 | 157.5091965 | 3.96173E−36 | 3.88153E−34 |
| Gm9843 | 0.337819429 | 10.49171063 | 156.9213278 | 5.32526E−36 | 5.18603E−34 |
| Vim | −0.444021646 | 10.47382906 | 156.4739419 | 6.66963E−36 | 6.45636E−34 |
| Hspe1 | 0.476979188 | 10.27539148 | 156.3922483 | 6.9495E−36 | 6.68724E−34 |
| Pkm | −0.357373771 | 10.1621659 | 155.5786776 | 1.04649E−35 | 1.00104E−33 |
| Ccnd2 | 0.45234154 | 9.824519901 | 155.4920344 | 1.09313E−35 | 1.0395E−33 |
| Kpna1 | 0.56949201 | 9.374027049 | 155.1833538 | 1.27681E−35 | 1.20707E−33 |
| D16Ertd472e | 0.457345281 | 9.624068057 | 154.0966474 | 2.20601E−35 | 2.07339E−33 |
| S100a10 | −0.30141114 | 10.94016781 | 154.0320192 | 2.27893E−35 | 2.12955E−33 |
| Ccnb2 | −0.632375053 | 9.212365668 | 153.0418621 | 3.7508E−35 | 3.4848E−33 |
| Arf5 | −0.352340754 | 9.981775241 | 152.9596822 | 3.90917E−35 | 3.61118E−33 |
| Wdr1 | −0.508723478 | 9.566638472 | 150.3847378 | 1.42845E−34 | 1.31207E−32 |
| Slamf6 | −0.73893756 | 9.319065378 | 149.892996 | 1.82957E−34 | 1.671E−32 |
| Nfkb2 | 0.481830243 | 9.521885906 | 149.3244526 | 2.43568E−34 | 2.21209E−32 |
| Ctla4 | 0.427747962 | 10.1530728 | 149.2551209 | 2.52217E−34 | 2.27785E−32 |
| Gm26740 | 0.475205161 | 9.65890753 | 147.7317529 | 5.42961E−34 | 4.8764E−32 |
| Ube2c | −0.767726489 | 9.233532012 | 147.2871077 | 6.79154E−34 | 6.06586E−32 |
| Ago2 | 0.552705416 | 9.333716732 | 147.1394927 | 7.31539E−34 | 6.49784E−32 |
| Birc5 | −0.876229377 | 9.251711307 | 146.9093125 | 8.21402E−34 | 7.25617E−32 |
| Gapdh | −0.299112432 | 10.56241857 | 146.8533552 | 8.44868E−34 | 7.4229E−32 |
| Tagln2 | −0.374638047 | 10.29890981 | 145.9056211 | 1.36136E−33 | 1.18961E−31 |
| H2-Q6 | 0.403745938 | 9.740233488 | 145.8383905 | 1.40822E−33 | 1.22394E−31 |
| Slc25a4 | −0.505941443 | 9.597149244 | 145.6070287 | 1.58216E−33 | 1.36777E−31 |
| Limd2 | −0.388646178 | 10.08900365 | 145.4829362 | 1.68415E−33 | 1.44819E−31 |
| Eif3h | −0.278504501 | 10.29703998 | 144.4345701 | 2.85483E−33 | 2.44186E−31 |
| Acadl | 0.535803555 | 9.426500038 | 143.2243213 | 5.2503E−33 | 4.46718E−31 |
| Set | −0.454430557 | 9.773657326 | 143.1546396 | 5.43776E−33 | 4.60245E−31 |
| Ifi27l2a | −0.716236939 | 9.817229705 | 143.0476155 | 5.73879E−33 | 4.83194E−31 |
| Naca | −0.227053893 | 10.80149233 | 142.6503305 | 7.00947E−33 | 5.87125E−31 |
| Ccr2 | 0.502477166 | 9.897669405 | 142.4515995 | 7.74707E−33 | 6.45563E−31 |
| Cox5b | −0.342361485 | 10.0435119 | 142.0600531 | 9.43518E−33 | 7.82201E−31 |
| Atp5l | −0.258434484 | 10.47768281 | 141.8235363 | 1.06284E−32 | 8.76623E−31 |
| Crot | 0.556863942 | 9.397231084 | 141.2860124 | 1.39316E−32 | 1.14324E−30 |
| Batf | −0.546937194 | 9.653590189 | 140.6727645 | 1.89713E−32 | 1.54894E−30 |
| Prelid1 | −0.441857874 | 9.693857441 | 140.6149759 | 1.95315E−32 | 1.58053E−30 |
| Tmem243 | 0.522419335 | 9.382999777 | 140.6127047 | 1.95538E−32 | 1.58053E−30 |
| Tnfsf8 | −0.746667051 | 10.01800671 | 139.9167141 | 2.77604E−32 | 2.23271E−30 |
| Hnrnpa3 | −0.33187099 | 10.15611501 | 139.2100176 | 3.96249E−32 | 3.17117E−30 |
| Cd7 | −0.955133649 | 9.49407751 | 138.7709549 | 4.94294E−32 | 3.93634E−30 |
| Dtx4 | 0.588912666 | 9.260819691 | 138.0520211 | 7.09925E−32 | 5.62581E−30 |
| H2-Q4 | 0.389274444 | 9.753654107 | 136.5636584 | 1.5022E−31 | 1.18461E−29 |
| Cd53 | 0.267838459 | 10.54705558 | 136.2510731 | 1.75831E−31 | 1.37984E−29 |
| Cirbp | 0.372879235 | 9.821591303 | 136.0340404 | 1.96139E−31 | 1.53178E−29 |
| Vcpkmt | 0.490218515 | 9.468573275 | 135.7031786 | 2.31703E−31 | 1.80082E−29 |
| Ms4a6b | −0.363533853 | 10.19483269 | 135.0862488 | 3.16134E−31 | 2.44527E−29 |
| Rps23 | 0.136676892 | 12.485355 | 134.6595977 | 3.91917E−31 | 3.01701E−29 |
| Tnfrsf18 | 0.363350646 | 10.06784669 | 134.0909261 | 5.21897E−31 | 3.99858E−29 |
| Erh | −0.424916553 | 9.741395893 | 134.0299115 | 5.38185E−31 | 4.10392E−29 |
| Ran | −0.43677254 | 9.801466447 | 132.078733 | 1.43801E−30 | 1.09141E−28 |
| Pnrc1 | 0.279551131 | 10.57763129 | 131.7585909 | 1.68966E−30 | 1.27641E−28 |
| Tox2 | −0.494429465 | 9.191330625 | 130.5732514 | 3.06992E−30 | 2.30829E−28 |
| Rpl23a-ps3 | 0.275124423 | 10.72027491 | 130.4319429 | 3.29643E−30 | 2.46713E−28 |
| Cytip | 0.305917579 | 10.18851993 | 130.0553827 | 3.98502E−30 | 2.96875E−28 |
| Eomes | −0.808402797 | 9.215475259 | 129.9784094 | 4.14259E−30 | 3.07198E−28 |
| Gnb1 | −0.441806484 | 9.703176458 | 129.6601205 | 4.86309E−30 | 3.5898E−28 |
| Calm2 | 0.281457442 | 10.41344741 | 129.5654599 | 5.10063E−30 | 3.74803E−28 |
| Cdk6 | −0.652717771 | 9.323625219 | 128.9918874 | 6.80961E−30 | 4.98119E−28 |
| Plscr1 | 0.538048062 | 9.521580156 | 128.9205551 | 7.05879E−30 | 5.1402E−28 |
| Ldha | −0.30771685 | 10.3562952 | 128.8545946 | 7.29731E−30 | 5.29006E−28 |
| Btg2 | 0.325367246 | 10.53756271 | 128.1980853 | 1.01582E−29 | 7.3311E−28 |
| Lmnb1 | 0.417613687 | 9.781595274 | 127.9843878 | 1.13129E−29 | 8.12822E−28 |
| Rgcc | 0.688439061 | 9.563499202 | 127.9722539 | 1.13823E−29 | 8.14188E−28 |
| mt-Co2 | −0.195884131 | 11.38674194 | 127.8524506 | 1.20905E−29 | 8.61038E−28 |
| Rpl37 | 0.14371841 | 12.07306348 | 127.7719313 | 1.25911E−29 | 8.92756E−28 |
| Tpt1 | 0.157950068 | 11.78349556 | 127.3775638 | 1.5359E−29 | 1.08425E−27 |
| Gramd3 | 0.412706804 | 10.20636682 | 127.0334653 | 1.82667E−29 | 1.28391E−27 |
| Gpx4 | 0.323281731 | 10.07618977 | 126.6694303 | 2.19444E−29 | 1.53573E−27 |
| Cdca3 | −0.472652508 | 9.196810536 | 126.3015826 | 2.64133E−29 | 1.84051E−27 |
| Ccr5 | 0.540398603 | 9.51436215 | 125.9629608 | 3.13276E−29 | 2.17357E−27 |
| Xist | 0.750105813 | 9.888938449 | 125.3127903 | 4.34727E−29 | 3.00333E−27 |
| Amz2 | 0.525949285 | 9.343188021 | 125.1580219 | 4.6999E−29 | 3.23313E−27 |
| St8sia4 | 0.546224409 | 9.30811702 | 125.1322589 | 4.76131E−29 | 3.2615E−27 |
| Zfp36 | −0.811141834 | 9.294562058 | 124.0351838 | 8.2763E−29 | 5.64534E−27 |
| Gltp | −0.507531701 | 9.448093847 | 122.3124798 | 1.972E−28 | 1.33947E−26 |
| Rps29 | 0.16387477 | 12.24471453 | 121.9208529 | 2.40233E−28 | 1.61844E−26 |
| mt-Cytb | −0.234943587 | 11.07879665 | 121.9205166 | 2.40274E−28 | 1.61844E−26 |
| Igtp | 0.504055964 | 9.509084729 | 121.2284811 | 3.40562E−28 | 2.28445E−26 |
| Tbx21 | 0.557575631 | 9.277655891 | 120.9884633 | 3.84361E−28 | 2.56759E−26 |
| Capn2 | −0.559140819 | 9.322209356 | 120.5648275 | 4.75861E−28 | 3.16575E−26 |
| Tnfaip8 | 0.423063549 | 9.630282095 | 120.3926675 | 5.19002E−28 | 3.4386E−26 |
| Cox6b1 | −0.278552491 | 10.15777506 | 118.9346766 | 1.08236E−27 | 7.1418E−26 |
| Arpc4 | −0.35503697 | 9.810411465 | 118.1622595 | 1.59769E−27 | 1.04993E−25 |
| Ncl | 0.289826807 | 10.07288426 | 116.7834774 | 3.20182E−27 | 2.09557E−25 |
| Anp32b | −0.444708978 | 9.652821361 | 116.7595092 | 3.24075E−27 | 2.1125E−25 |
| 2700094K13Rik | −0.564948118 | 9.385390715 | 116.5920387 | 3.52629E−27 | 2.2894E−25 |
| Mfsd6 | 0.50695786 | 9.369441943 | 116.3646697 | 3.95464E−27 | 2.55723E−25 |
| Tuba4a | 0.505604749 | 9.389389134 | 116.122805 | 4.46758E−27 | 2.8774E−25 |
| Psmb3 | −0.320343547 | 9.906091127 | 116.0098837 | 4.72934E−27 | 3.03391E−25 |
| Gcnt2 | 0.569564724 | 9.21801522 | 115.8092096 | 5.23294E−27 | 3.3437E−25 |
| Bcl2l1 | 0.52714474 | 9.376464751 | 115.7519424 | 5.38625E−27 | 3.42812E−25 |
| Rpl23a | 0.13563776 | 12.17564937 | 115.2767761 | 6.84457E−27 | 4.33919E−25 |
| Rilpl2 | −0.546494215 | 9.398901147 | 115.0842673 | 7.54234E−27 | 4.74599E−25 |
| Hcst | 0.252834434 | 10.61089101 | 115.0835741 | 7.54497E−27 | 4.74599E−25 |
| Ass1 | −0.51973323 | 9.534398406 | 114.7355685 | 8.99234E−27 | 5.6345E−25 |
| Ubb | 0.210452543 | 12.90465659 | 114.6470029 | 9.40307E−27 | 5.86911E−25 |
| Rpl12 | 0.207576935 | 10.99249069 | 113.9508319 | 1.3358E−26 | 8.30559E−25 |
| Ywhah | −0.3943071 | 9.722022338 | 113.8003544 | 1.44112E−26 | 8.92609E−25 |
| Emp1 | 0.514205699 | 9.358540577 | 113.4343025 | 1.7333E−26 | 1.06949E−24 |
| Hint1 | −0.296617099 | 10.08286375 | 113.2323232 | 1.91917E−26 | 1.17967E−24 |
| Dkkl1 | 0.504724291 | 9.308686473 | 113.1616116 | 1.98885E−26 | 1.21787E−24 |
| Tcp11l2 | 0.490513451 | 9.433883691 | 112.7141682 | 2.49235E−26 | 1.52043E−24 |
| Xcl1 | 1.194998958 | 9.40506854 | 112.2843688 | 3.09567E−26 | 1.88137E−24 |
| Rpl24 | −0.148371665 | 11.77231549 | 111.9741934 | 3.61992E−26 | 2.19174E−24 |
| Antxr2 | 0.524111919 | 9.257858404 | 111.8224562 | 3.90784E−26 | 2.35725E−24 |
| Tnfrsf25 | −0.601839819 | 9.258889969 | 111.8014874 | 3.94939E−26 | 2.37345E−24 |
| Timp2 | −0.576983903 | 9.295775851 | 110.8832755 | 6.27592E−26 | 3.75765E−24 |
| Amd1 | 0.506986556 | 9.33704939 | 110.5121875 | 7.56787E−26 | 4.51447E−24 |
| Hsd11b1 | −0.573712404 | 9.289649192 | 109.2285995 | 1.44609E−25 | 8.59464E−24 |
| Cyba | −0.255410775 | 10.4309546 | 108.8830417 | 1.7215E−25 | 1.01941E−23 |
| Twf2 | −0.506092699 | 9.389135379 | 108.4415119 | 2.15105E−25 | 1.26912E−23 |
| Cldnd1 | 0.437135874 | 9.795121381 | 108.1944154 | 2.43665E−25 | 1.4324E−23 |
| H3f3b | 0.176835587 | 12.3442743 | 107.9279532 | 2.78728E−25 | 1.63258E−23 |
| Sub1 | 0.223420381 | 11.1488509 | 107.9028414 | 2.82282E−25 | 1.63562E−23 |
| Nme1 | −0.410983186 | 9.653644082 | 107.9169334 | 2.80282E−25 | 1.63562E−23 |
| Dbi | −0.503923317 | 9.46225676 | 107.9081308 | 2.8153E−25 | 1.63562E−23 |
| Mat2a | 0.442640021 | 9.501583097 | 107.6673044 | 3.17904E−25 | 1.82891E−23 |
| Emb | 0.3409222 | 10.25108753 | 107.6709239 | 3.17324E−25 | 1.82891E−23 |
| Itga4 | −0.538393532 | 9.394382328 | 107.19807 | 4.02829E−25 | 2.30927E−23 |
| Tpgs1 | −0.547232328 | 9.260754207 | 106.3574922 | 6.15642E−25 | 3.51678E−23 |
| Eno1 | −0.340480149 | 9.942475304 | 106.210237 | 6.63132E−25 | 3.77472E−23 |
| Sash3 | −0.558983887 | 9.280791065 | 105.5651388 | 9.18291E−25 | 5.2088E−23 |
| Rpl21 | 0.141624442 | 11.76877751 | 105.5424416 | 9.2887E−25 | 5.25039E−23 |
| Rpl18a | 0.115781407 | 12.84271355 | 105.3725254 | 1.01203E−24 | 5.70054E−23 |
| Bak1 | −0.530049389 | 9.291925002 | 104.9557381 | 1.24895E−24 | 7.01059E−23 |
| Tmpo | −0.551234966 | 9.399332732 | 104.4649178 | 1.60001E−24 | 8.95012E−23 |
| Fkbp1a | −0.359503623 | 9.733415831 | 104.2324114 | 1.79923E−24 | 1.00298E−22 |
| Ndfip1 | 0.349750518 | 9.658014518 | 103.9167295 | 2.11E−24 | 1.17218E−22 |
| Rgs1 | 0.371335135 | 11.08975907 | 103.6582181 | 2.40408E−24 | 1.33097E−22 |
| Ech1 | −0.423155488 | 9.539595739 | 103.6369821 | 2.42999E−24 | 1.34072E−22 |
| Nop56 | 0.424122186 | 9.44999363 | 103.4998715 | 2.60411E−24 | 1.43191E−22 |
| Il22 | −1.546604232 | 9.482887499 | 103.4238643 | 2.70596E−24 | 1.48286E−22 |
| Gnb2 | −0.326724769 | 9.816182555 | 102.9851985 | 3.37662E−24 | 1.84413E−22 |
| Psma4 | −0.420490298 | 9.543577958 | 102.9024256 | 3.52068E−24 | 1.91634E−22 |
| Coq10b | 0.407565103 | 9.509829344 | 102.7580597 | 3.78682E−24 | 2.05429E−22 |
| Kdm6b | −0.564998666 | 9.740092853 | 102.2484393 | 4.89775E−24 | 2.64806E−22 |
| Def6 | −0.512941554 | 9.346043162 | 100.9911559 | 9.23951E−24 | 4.97886E−22 |
| Rpl9-ps6 | 0.349639434 | 9.841114929 | 100.3147377 | 1.30006E−23 | 6.95919E−22 |
| Gpr171 | 0.366635846 | 9.722718651 | 100.3157828 | 1.29937E−23 | 6.95919E−22 |
| Rpl9 | 0.128286541 | 11.84230049 | 100.2331707 | 1.35472E−23 | 7.22783E−22 |
| Rpl27-ps3 | 0.364109983 | 9.836595897 | 100.0840856 | 1.46062E−23 | 7.76726E−22 |
| Srsf5 | 0.266057713 | 10.09087953 | 99.90009247 | 1.60282E−23 | 8.49546E−22 |
| Mki67 | −0.465243552 | 9.203073676 | 99.69938332 | 1.77376E−23 | 9.37081E−22 |
| Cisd3 | −0.525536963 | 9.289876433 | 99.52839188 | 1.93371E−23 | 1.01825E−21 |
| Arid5a | 0.364870228 | 9.656742664 | 99.48865318 | 1.9729E−23 | 1.03552E−21 |
| Fau | 0.117665937 | 12.30167106 | 99.0301156 | 2.48691E−23 | 1.30108E−21 |
| Nr4a3 | 0.496623394 | 9.253599057 | 98.98547036 | 2.54361E−23 | 1.32645E−21 |
| Klf6 | −0.463656693 | 9.896878744 | 98.88672106 | 2.67366E−23 | 1.38979E−21 |
| Rpl23 | 0.143460142 | 11.70879327 | 98.81011908 | 2.77911E−23 | 1.43997E−21 |
| Cdkn3 | −0.295491219 | 9.177389757 | 98.76406715 | 2.84449E−23 | 1.46914E−21 |
| Taf10 | −0.439262723 | 9.515336126 | 98.5028028 | 3.24565E−23 | 1.67099E−21 |
| Acp5 | −0.483724271 | 9.39691276 | 98.31170727 | 3.57446E−23 | 1.83444E−21 |
| Alyref | −0.512386856 | 9.471617456 | 98.26781953 | 3.65457E−23 | 1.86961E−21 |
| Mtch1 | −0.522764482 | 9.286913269 | 97.80966112 | 4.60595E−23 | 2.34889E−21 |
| Malat1 | 0.220170852 | 14.6684552 | 97.38689353 | 5.70222E−23 | 2.89881E−21 |
| Rps14 | 0.110659316 | 12.65498594 | 97.1959583 | 6.27947E−23 | 3.18225E−21 |
| Tk1 | −0.352993071 | 9.186456549 | 97.00099196 | 6.92926E−23 | 3.50058E−21 |
| Rpl36a | 0.183239744 | 11.04535005 | 96.7894076 | 7.71075E−23 | 3.88324E−21 |
| Icos | 0.315321976 | 10.25650888 | 96.68836511 | 8.11447E−23 | 4.07387E−21 |
| Serf2 | −0.187728813 | 10.7068503 | 96.04038582 | 1.12564E−22 | 5.63376E−21 |
| Tnfsf11 | 0.557938441 | 9.547078237 | 95.67026522 | 1.35703E−22 | 6.77093E−21 |
| Aprt | −0.395206011 | 9.637373978 | 95.66236935 | 1.36246E−22 | 6.77707E−21 |
| Lamtor5 | −0.44478946 | 9.410023241 | 95.65457018 | 1.36784E−22 | 6.78295E−21 |
| Sec62 | 0.310396292 | 9.756891332 | 95.09887676 | 1.81109E−22 | 8.95356E−21 |
| Glul | 0.464844545 | 9.375643512 | 94.1625981 | 2.90641E−22 | 1.43247E−20 |
| Hmgn2 | −0.576602296 | 9.445404975 | 94.09059207 | 3.01408E−22 | 1.48102E−20 |
| Grap2 | 0.454216031 | 9.308784996 | 94.05858529 | 3.06322E−22 | 1.50061E−20 |
| Hspa5 | 0.244336232 | 10.97867799 | 94.03946443 | 3.09295E−22 | 1.51059E−20 |
| Gm2a | −0.430040205 | 9.581841921 | 93.88699024 | 3.34063E−22 | 1.62664E−20 |
| Izumo1r | −0.608712826 | 9.250980816 | 93.71023726 | 3.65267E−22 | 1.77324E−20 |
| Adrb2 | 0.535741715 | 9.281420121 | 93.6761559 | 3.71611E−22 | 1.79864E−20 |
| Traf1 | 0.282213829 | 10.10515014 | 92.36351394 | 7.21318E−22 | 3.48084E−20 |
| Ptpn7 | −0.519506241 | 9.307713029 | 92.22828093 | 7.72331E−22 | 3.71593E−20 |
| Crbn | 0.358418487 | 9.708112889 | 92.04198204 | 8.48571E−22 | 4.07062E−20 |
| Calm3 | −0.363800232 | 9.648589796 | 92.00946041 | 8.62631E−22 | 4.12583E−20 |
| Snrpe | −0.308597904 | 9.827468486 | 91.71067545 | 1.00322E−21 | 4.7841E−20 |
| Peli1 | 0.331773787 | 9.75612779 | 91.23737361 | 1.27431E−21 | 6.05895E−20 |
| Cdkn2d | −0.507234255 | 9.369237934 | 91.08746139 | 1.3746E−21 | 6.51665E−20 |
| 1500009L16Rik | −0.548390626 | 9.247085147 | 91.06913756 | 1.38739E−21 | 6.55805E−20 |
| Saraf | 0.265182173 | 10.21648935 | 91.0409854 | 1.40727E−21 | 6.63262E−20 |
| Me2 | 0.460112182 | 9.315619916 | 90.96509981 | 1.46229E−21 | 6.8719E−20 |
| Tnfrsf9 | 0.580005635 | 9.402134791 | 90.77972333 | 1.60591E−21 | 7.52495E−20 |
| Ccna2 | −0.400426756 | 9.19384283 | 90.75730099 | 1.62421E−21 | 7.58871E−20 |
| Mrpl42 | −0.491601474 | 9.309100457 | 90.67135954 | 1.69631E−21 | 7.90275E−20 |
| Socs1 | 0.473388021 | 9.398189402 | 89.78506933 | 2.6549E−21 | 1.23331E−19 |
| Nusap1 | −0.513197969 | 9.207259227 | 89.5987643 | 2.91706E−21 | 1.35121E−19 |
| Atp5f1 | −0.299068276 | 9.82227645 | 89.58192797 | 2.94199E−21 | 1.35886E−19 |
| Dut | −0.525005434 | 9.370492812 | 89.35609919 | 3.29774E−21 | 1.51884E−19 |
| Rpl22l1 | −0.210893716 | 10.87576972 | 89.25595861 | 3.46896E−21 | 1.59316E−19 |
| Oat | 0.438985505 | 9.329297018 | 89.15554471 | 3.64959E−21 | 1.67137E−19 |
| Wtap | −0.440428643 | 9.407943162 | 89.1100865 | 3.73442E−21 | 1.70539E−19 |
| Arpc5l | −0.465637304 | 9.354510705 | 88.86346776 | 4.23024E−21 | 1.92637E−19 |
| Reep5 | −0.359793383 | 9.637232149 | 88.39947996 | 5.34849E−21 | 2.42875E−19 |
| Rps18-ps3 | 0.402507463 | 9.485009861 | 88.29003132 | 5.65276E−21 | 2.55973E−19 |
| Eif3f | −0.187506949 | 10.66456064 | 88.11582239 | 6.17319E−21 | 2.78759E−19 |
| Anxa6 | −0.329415877 | 9.7471733 | 87.93028771 | 6.78025E−21 | 3.05319E−19 |
| Rpl36-ps3 | 0.382762365 | 9.494055337 | 87.66475526 | 7.75439E−21 | 3.48215E−19 |
| Tob1 | 0.4322116 | 9.42000657 | 87.56689353 | 8.14771E−21 | 3.64864E−19 |
| Eif1ax | −0.447944725 | 9.373714585 | 87.11118469 | 1.02589E−20 | 4.58135E−19 |
| Itpkb | 0.388213113 | 9.50956752 | 86.97951369 | 1.09651E−20 | 4.88325E−19 |
| Rps11 | −0.120222506 | 12.26312327 | 86.95742035 | 1.10883E−20 | 4.92454E−19 |
| Ccr7 | −0.827352902 | 9.273177568 | 86.88147711 | 1.15223E−20 | 5.1033E−19 |
| Rps13 | 0.118476361 | 11.9252863 | 86.86801277 | 1.16011E−20 | 5.12412E−19 |
| Sytl3 | 0.385802963 | 9.504493169 | 85.81774359 | 1.97308E−20 | 8.69122E−19 |
| Hnrnpf | −0.218975824 | 10.40312097 | 85.6906052 | 2.1041E−20 | 9.24318E−19 |
| Relb | 0.381710195 | 9.473542129 | 85.55433278 | 2.25422E−20 | 9.8758E−19 |
| Prdx1 | −0.30210979 | 9.945243865 | 85.47656524 | 2.34464E−20 | 1.02442E−18 |
| Hif1a | 0.31517707 | 9.774227368 | 85.08751446 | 2.85447E−20 | 1.24381E−18 |
| Cox5a | −0.258557899 | 10.08167074 | 84.9467374 | 3.06512E−20 | 1.33201E−18 |
| Rasgrp2 | −0.489347096 | 9.377108103 | 84.74049136 | 3.40211E−20 | 1.47449E−18 |
| Tob2 | 0.453132367 | 9.437008471 | 84.68313645 | 3.50225E−20 | 1.51383E−18 |
| Asf1b | −0.504596732 | 9.223323692 | 84.66123288 | 3.54126E−20 | 1.52661E−18 |
| Id2 | 0.312337499 | 10.64556525 | 84.33429136 | 4.17805E−20 | 1.79634E−18 |

TABLE 3C
LPL-2 vs. All

| GeneSymbol | logFC | logCPM | LR | PValue | FDR |
|---|---|---|---|---|---|
| Lgals1 | 1.107635326 | 11.62794788 | 1745.765645 | 0 | 0 |
| Crip1 | 1.160964893 | 11.66677342 | 1329.701702 | 3.969E−291 | 3.2081E−287 |
| S100a6 | 1.198020946 | 10.99644365 | 1210.536111 | 3.1298E−265 | 1.6865E−261 |
| S100a10 | 0.711665053 | 10.94013071 | 1098.436026 | 7.2245E−241 | 2.9198E−237 |
| Lgals3 | 1.396672722 | 10.20022841 | 1079.543841 | 9.2247E−237 | 2.9825E−233 |
| S100a4 | 1.527217655 | 9.866777731 | 973.9490277 | 8.2582E−214 | 2.225E−210 |
| Itgb1 | 1.751523134 | 9.299279016 | 928.1234969 | 7.5546E−204 | 1.7447E−200 |
| Emp3 | 0.930153302 | 9.864694656 | 866.1214647 | 2.2739E−190 | 4.5949E−187 |
| Actg1 | 0.557655647 | 12.09959146 | 800.9549005 | 3.3454E−176 | 6.0091E−173 |
| Vim | 0.882078214 | 10.4737473 | 792.3381022 | 2.4998E−174 | 4.0412E−171 |
| Ifitm1 | 3.164079151 | 9.599747165 | 776.5403323 | 6.8031E−171 | 9.9982E−168 |
| Rps27 | −0.387759423 | 13.23483519 | 632.9819097 | 1.1228E−139 | 1.5125E−136 |
| Rps29 | −0.402671414 | 12.24470733 | 625.94336 | 3.8116E−138 | 4.7399E−135 |
| Itgb7 | 0.728056874 | 9.956612648 | 623.3865772 | 1.3715E−137 | 1.5837E−134 |
| Ms4a4b | 0.643490527 | 10.75178795 | 622.8012833 | 1.8386E−137 | 1.9815E−134 |
| Junb | −0.777993669 | 11.89087159 | 602.8382153 | 4.0408E−133 | 4.0827E−130 |
| Pglyrp1 | 1.2119906 | 9.331208685 | 570.368253 | 4.6689E−126 | 4.4398E−123 |
| Lsp1 | 0.607948171 | 10.3214748 | 559.4531755 | 1.1055E−123 | 9.9289E−121 |
| Cd52 | 0.479542234 | 11.79358704 | 526.2289603 | 1.8679E−116 | 1.5893E−113 |
| Klrd1 | 1.281868785 | 9.372635779 | 515.4102303 | 4.2181E−114 | 3.4095E−111 |
| Got1 | −1.246704373 | 9.855927617 | 502.3476321 | 2.9321E−111 | 2.2571E−108 |
| Odc1 | −0.999359158 | 10.45374948 | 480.1976985 | 1.9352E−106 | 1.422E−103 |
| Nkg7 | 0.635156724 | 11.21006764 | 466.4812842 | 1.8684E−103 | 1.3133E−100 |
| Cd48 | 0.801758485 | 9.721230863 | 465.5348676 | 3.0021E−103 | 2.0222E−100 |
| Glipr2 | 0.840279767 | 9.484133594 | 428.4566632 | 3.52206E−95 | 2.2775E−92 |
| Anxa1 | 1.198698478 | 9.278573422 | 424.2938572 | 2.83693E−94 | 1.76392E−91 |
| Klf2 | 1.251446468 | 9.96061809 | 422.5102224 | 6.93538E−94 | 4.1525E−91 |
| Pycard | 0.821577501 | 9.577165063 | 416.4397023 | 1.45343E−92 | 8.3915E−90 |
| Rps28 | −0.384870366 | 11.70752993 | 413.5454065 | 6.19998E−92 | 3.45617E−89 |
| AA467197 | 0.966906994 | 9.471388865 | 399.1453036 | 8.45263E−89 | 4.55484E−86 |
| Ramp3 | −1.423901616 | 9.937109715 | 376.5001514 | 7.19354E−84 | 3.75131E−81 |
| Rps27rt | −0.541894598 | 11.16203814 | 374.50616 | 1.9547E−83 | 9.87488E−81 |
| Fxyd5 | 0.374291229 | 10.88895574 | 373.588429 | 3.09665E−83 | 1.51698E−80 |
| S1pr4 | 0.789331367 | 9.466032912 | 359.098318 | 4.4251E−80 | 2.10401E−77 |
| Rgs1 | −0.786014575 | 11.08978155 | 352.5170907 | 1.19954E−78 | 5.54048E−76 |
| Bin2 | 0.674444277 | 9.612834003 | 332.5449837 | 2.68221E−74 | 1.20446E−71 |
| Zyx | 0.871870874 | 9.311172942 | 332.0349778 | 3.46394E−74 | 1.51346E−71 |
| Anxa2 | 0.694314803 | 9.842643949 | 325.6200897 | 8.64484E−73 | 3.67769E−70 |
| Tnfrsf4 | −0.770960782 | 10.62243251 | 323.315614 | 2.746E−72 | 1.13825E−69 |
| Ifitm2 | 0.899150526 | 10.68445772 | 319.4990272 | 1.86219E−71 | 7.52605E−69 |
| Ctla4 | −0.717492 | 10.15306486 | 313.1390335 | 4.52291E−70 | 1.78335E−67 |
| Malat1 | −0.429232236 | 14.66848179 | 310.0933732 | 2.08393E−69 | 8.02116E−67 |
| Crem | −0.936462251 | 10.06717199 | 309.4765776 | 2.83955E−69 | 1.06754E−66 |
| Ifitm3 | 1.07385109 | 9.717589617 | 305.0557418 | 2.60811E−68 | 9.58243E−66 |
| Myl6 | 0.319639864 | 10.93131338 | 295.8728592 | 2.6119E−66 | 9.38311E−64 |
| Gna15 | 0.723530518 | 9.413846926 | 293.4396749 | 8.85314E−66 | 3.1113E−63 |
| Reep5 | 0.576970257 | 9.637214739 | 287.0003799 | 2.23954E−64 | 7.70305E−62 |
| Rpl12 | −0.361118726 | 10.9924655 | 286.9340944 | 2.31527E−64 | 7.79763E−62 |
| S1pr1 | 0.88219731 | 9.290345556 | 285.4522276 | 4.86969E−64 | 1.6066E−61 |
| Arhgdib | 0.369730687 | 10.73181656 | 282.2713569 | 2.40234E−63 | 7.76726E−61 |
| Rpl37a | −0.230463204 | 12.61563546 | 281.1880744 | 4.1371E−63 | 1.31138E−60 |
| S100a13 | 0.477912226 | 9.89789302 | 277.2675542 | 2.95838E−62 | 9.19714E−60 |
| Sh3bgrl3 | 0.316794291 | 11.24486223 | 275.9719088 | 5.66773E−62 | 1.72877E−59 |
| Rpl36a | −0.336268091 | 11.04533644 | 275.3234377 | 7.84748E−62 | 2.3493E−59 |
| Hspe1 | −0.734968089 | 10.27537472 | 273.5809707 | 1.88136E−61 | 5.52982E−59 |
| Cd47 | 0.409603749 | 10.23585118 | 273.0362546 | 2.47277E−61 | 7.13837E−59 |
| Ly6c2 | 1.596385353 | 9.266014145 | 270.6111246 | 8.3506E−61 | 2.36835E−58 |
| Rac2 | 0.302952162 | 11.09602966 | 268.3984579 | 2.53491E−60 | 7.0654E−58 |
| Rps16 | −0.212403538 | 12.35142328 | 267.4464731 | 4.08742E−60 | 1.11995E−57 |
| Klrc1 | 0.869453849 | 9.658428758 | 264.6488216 | 1.66425E−59 | 4.48404E−57 |
| Tspo | 0.452765846 | 9.918102726 | 261.1004221 | 9.87762E−59 | 2.61773E−56 |
| Ptma | −0.368146026 | 11.28837461 | 260.25122 | 1.51271E−58 | 3.94427E−56 |
| Klrk1 | 0.967738565 | 9.564428224 | 260.2132923 | 1.54178E−58 | 3.95627E−56 |
| Ubb | −0.340558743 | 12.90467053 | 253.8842203 | 3.69547E−57 | 9.33454E−55 |
| Rps15a | −0.209512678 | 12.40382894 | 252.2257436 | 8.49603E−57 | 2.11303E−54 |
| Selplg | 0.412758765 | 10.21362445 | 248.0654969 | 6.85781E−56 | 1.67975E−53 |
| Coro1a | 0.365510663 | 10.77320156 | 247.1536687 | 1.08388E−55 | 2.61521E−53 |
| Rasgrp2 | 0.746165592 | 9.377101403 | 245.1227903 | 3.00439E−55 | 7.1425E−53 |
| Rplp2 | −0.239346191 | 11.91018134 | 243.9392817 | 5.44245E−55 | 1.27511E−52 |
| Ggt1 | 0.845090141 | 9.203353657 | 240.5208096 | 3.02793E−54 | 6.9928E−52 |
| Ctsd | 0.558572919 | 9.641720936 | 239.4727209 | 5.12479E−54 | 1.16687E−51 |
| Hsp90ab1 | −0.478995578 | 11.19701824 | 238.7640474 | 7.31478E−54 | 1.64237E−51 |
| Prr13 | 0.385685076 | 10.23778924 | 236.1715783 | 2.68844E−53 | 5.9536E−51 |
| Klhl6 | 0.724094678 | 9.316899465 | 233.3735902 | 1.09557E−52 | 2.39338E−50 |
| Rps20 | −0.30849135 | 11.06878098 | 226.7801872 | 3.00297E−51 | 6.4728E−49 |
| Anxa6 | 0.477668152 | 9.747152467 | 226.6718002 | 3.17095E−51 | 6.74495E−49 |
| Ms4a6b | 0.431526867 | 10.19485172 | 224.1053813 | 1.15062E−50 | 2.4157E−48 |
| Epsti1 | 0.481889636 | 9.946002487 | 222.8372549 | 2.17532E−50 | 4.50849E−48 |
| Tnfsf11 | −0.995325248 | 9.547066191 | 222.1985428 | 2.99803E−50 | 6.13496E−48 |
| Ccl5 | 0.805026235 | 13.31088043 | 221.9811923 | 3.34383E−50 | 6.75704E−48 |
| Vps37b | −0.498283973 | 10.60463013 | 221.6741013 | 3.90145E−50 | 7.78651E−48 |
| Arl6ip5 | 0.446676163 | 9.776818831 | 218.2461026 | 2.18253E−49 | 4.30278E−47 |
| Asb2 | −0.823122831 | 9.480734994 | 211.5725771 | 6.23419E−48 | 1.21424E−45 |
| Tagln2 | 0.425196214 | 10.29885571 | 211.0250542 | 8.20787E−48 | 1.57962E−45 |
| Fth1 | −0.300407416 | 11.58475892 | 209.3862439 | 1.8697E−47 | 3.55594E−45 |
| Rplp1 | −0.235120996 | 11.70338212 | 208.9022913 | 2.38428E−47 | 4.4819E−45 |
| Prkca | −0.804901317 | 9.47273378 | 207.833874 | 4.07816E−47 | 7.57788E−45 |
| Gadd45b | −0.794871222 | 9.727248868 | 207.396577 | 5.08014E−47 | 9.33244E−45 |
| Dusp1 | −0.761030155 | 9.939132795 | 205.1520683 | 1.56893E−46 | 2.84982E−44 |
| Rps24 | −0.211580124 | 12.0059312 | 198.8559219 | 3.71107E−45 | 6.6659E−43 |
| Rpl29 | 0.231642848 | 11.21438856 | 194.4462645 | 3.40302E−44 | 6.04542E−42 |
| Hsp90aa1 | −0.610066784 | 10.76858556 | 190.5798156 | 2.37559E−43 | 4.17432E−41 |
| Gzma | 1.156987494 | 10.72602929 | 190.2449752 | 2.81098E−43 | 4.88628E−41 |
| Socs3 | −0.775105706 | 9.492203988 | 183.9508242 | 6.65037E−42 | 1.14372E−39 |
| Gngt2 | −0.76920447 | 9.363427733 | 183.8492044 | 6.99892E−42 | 1.19099E−39 |
| Ahnak | 0.497917205 | 9.6486335 | 183.5158121 | 8.27591E−42 | 1.39363E−39 |
| Isg15 | −0.919606683 | 9.495169961 | 182.7671755 | 1.20575E−41 | 2.00951E−39 |
| Eif1 | −0.215075871 | 11.85890479 | 181.8854963 | 1.87824E−41 | 3.09833E−39 |
| Arpc1b | 0.279591712 | 10.55364386 | 181.858684 | 1.90373E−41 | 3.10866E−39 |
| Usmg5 | −0.411662014 | 10.06859105 | 179.6012963 | 5.92208E−41 | 9.57363E−39 |
| Arpc5 | 0.357914994 | 10.09992322 | 178.1056272 | 1.25618E−40 | 2.01063E−38 |
| Pfn1 | 0.266033705 | 12.14558689 | 176.9357799 | 2.26201E−40 | 3.58507E−38 |
| Rps27a | −0.185752928 | 11.80983865 | 173.8717911 | 1.0558E−39 | 1.6571E−37 |
| Ncf4 | 0.616931601 | 9.322576992 | 173.4282403 | 1.31961E−39 | 2.05124E−37 |
| Izumo1r | −0.833944275 | 9.250979762 | 173.23288 | 1.45583E−39 | 2.24143E−37 |
| Ddx5 | −0.273154117 | 11.19917209 | 171.9221905 | 2.8142E−39 | 4.29191E−37 |
| Tob2 | −0.749576508 | 9.437017288 | 170.2866433 | 6.40562E−39 | 9.67787E−37 |
| Nrp1 | 0.475718816 | 9.166737051 | 169.9443495 | 7.60889E−39 | 1.13894E−36 |
| Il21r | −0.505264995 | 10.19620078 | 168.9413091 | 1.26008E−38 | 1.86885E−36 |
| Fosl2 | −0.703183417 | 9.531613662 | 167.1288651 | 3.13532E−38 | 4.60778E−36 |
| Cnbp | −0.336972315 | 10.25780833 | 165.3131087 | 7.8147E−38 | 1.13813E−35 |
| Slc25a5 | 0.31094711 | 10.3940621 | 164.7439998 | 1.04048E−37 | 1.50182E−35 |
| Cyba | 0.29682531 | 10.43094576 | 164.7129212 | 1.05687E−37 | 1.51198E−35 |
| Rgs2 | −0.683188002 | 10.05998032 | 161.9432033 | 4.25694E−37 | 6.03664E−35 |
| Abracl | 0.369184781 | 9.935327557 | 160.800472 | 7.5641E−37 | 1.06332E−34 |
| Fus | −0.414749315 | 9.899258939 | 159.1748408 | 1.71371E−36 | 2.38827E−34 |
| Rps21 | −0.275761051 | 10.92385795 | 157.8355226 | 3.36186E−36 | 4.64512E−34 |
| Cnn2 | 0.399519194 | 9.90288659 | 157.5798679 | 3.82334E−36 | 5.23797E−34 |
| Dgat1 | −0.627039178 | 9.778835893 | 157.0134201 | 5.08414E−36 | 6.90674E−34 |
| AB124611 | 0.538688322 | 9.180449702 | 156.1726476 | 7.7614E−36 | 1.04559E−33 |
| Ctsw | 0.407627769 | 9.94349627 | 154.5480574 | 1.75775E−35 | 2.34842E−33 |
| Rom1 | 0.565412526 | 9.185573671 | 153.2571454 | 3.36569E−35 | 4.45982E−33 |
| Wdr89 | −0.438648459 | 10.42635108 | 151.6029863 | 7.73744E−35 | 1.01694E−32 |
| Fos | −1.040226793 | 9.517179661 | 151.265106 | 9.1716E−35 | 1.19571E−32 |
| 1500009L16Rik | 0.743494969 | 9.247062972 | 151.0129633 | 1.04125E−34 | 1.34663E−32 |
| Rasa3 | 0.62108061 | 9.237628541 | 150.0581948 | 1.6836E−34 | 2.16009E−32 |
| Serpinb6b | 0.578341369 | 9.622263112 | 148.5069318 | 3.67553E−34 | 4.65689E−32 |
| S100a11 | 0.301724985 | 10.60261496 | 148.500602 | 3.68725E−34 | 4.65689E−32 |
| Dusp10 | −0.723669213 | 9.475537805 | 148.3230479 | 4.03195E−34 | 5.05275E−32 |
| Furin | −0.608670033 | 10.38118207 | 147.4418599 | 6.2826E−34 | 7.81265E−32 |
| Rpl38 | −0.196298052 | 11.67459296 | 147.2648676 | 6.868E−34 | 8.47542E−32 |
| Lfng | 0.600318631 | 9.38679975 | 146.3855751 | 1.06918E−33 | 1.30942E−31 |
| Cdkn1a | −0.694573024 | 9.693100492 | 143.1294135 | 5.50726E−33 | 6.69401E−31 |
| Flna | 0.610624204 | 9.27107707 | 141.7573801 | 1.09883E−32 | 1.32565E−30 |
| Ube2s | −0.407475264 | 10.00303894 | 140.9661991 | 1.63657E−32 | 1.95976E−30 |
| Msn | 0.333987136 | 9.894256294 | 140.9427275 | 1.65602E−32 | 1.96847E−30 |
| Rps9 | −0.160448533 | 11.99541117 | 140.6491255 | 1.91985E−32 | 2.26542E−30 |
| Rps8 | −0.188998621 | 11.59272654 | 140.2001816 | 2.40679E−32 | 2.81944E−30 |
| Tuba1b | −0.648609572 | 9.730329249 | 139.675338 | 3.1348E−32 | 3.64584E−30 |
| Hspa8 | −0.295280678 | 12.33066824 | 139.3532611 | 3.68674E−32 | 4.25713E−30 |
| Slc25a4 | 0.446208668 | 9.597123846 | 136.8862867 | 1.27692E−31 | 1.45371E−29 |
| mt-Co1 | 0.180371584 | 12.21548846 | 136.8986462 | 1.269E−31 | 1.45371E−29 |
| BC021614 | 0.604666583 | 9.270921447 | 135.1654782 | 3.03767E−31 | 3.43406E−29 |
| Trib2 | −0.68443991 | 9.363798856 | 134.4595166 | 4.3347E−31 | 4.86631E−29 |
| Sdf4 | −0.390564968 | 9.917420235 | 133.745527 | 6.21069E−31 | 6.92427E−29 |
| Adgre5 | 0.508601548 | 9.38877853 | 132.9968145 | 9.0557E−31 | 1.0027E−28 |
| Rpl23a | −0.155302608 | 12.17563324 | 132.7561574 | 1.02227E−30 | 1.11663E−28 |
| Hsph1 | −0.728156382 | 9.472375617 | 132.7619407 | 1.0193E−30 | 1.11663E−28 |
| Saraf | −0.355176401 | 10.21648742 | 131.5242654 | 1.90136E−30 | 2.06291E−28 |
| Arhgap31 | −0.552171788 | 9.654221563 | 131.071069 | 2.38898E−30 | 2.57469E−28 |
| Hspd1 | −0.558182167 | 9.781137532 | 130.015877 | 4.06513E−30 | 4.35211E−28 |
| Hmha1 | 0.381765719 | 9.750126154 | 129.5912318 | 5.03483E−30 | 5.35481E−28 |
| Atp1b3 | 0.486515092 | 9.413034296 | 129.2689007 | 5.92258E−30 | 6.25781E−28 |
| Cd27 | −0.803947605 | 9.342803037 | 126.3516604 | 2.57551E−29 | 2.70362E−27 |
| Tnfsf8 | −0.737355758 | 10.01797843 | 126.0626608 | 2.97926E−29 | 3.10727E−27 |
| Il22 | −1.756753656 | 9.482889609 | 126.0406633 | 3.01247E−29 | 3.12177E−27 |
| Ubald2 | −0.351497132 | 10.62004401 | 124.5294437 | 6.45147E−29 | 6.64296E−27 |
| Rpl36 | −0.164380166 | 11.87412221 | 124.1971999 | 7.62739E−29 | 7.80407E−27 |
| Ifi27l2a | −0.68890883 | 9.81720141 | 123.5990198 | 1.0311E−28 | 1.04835E−26 |
| Rpl23a-ps3 | −0.295698901 | 10.72022914 | 123.4369527 | 1.11886E−28 | 1.13046E−26 |
| Gas7 | 0.571971199 | 9.194248976 | 121.9870221 | 2.32353E−28 | 2.33305E−26 |
| Nccrp1 | 0.393930301 | 9.164828209 | 121.8725845 | 2.46149E−28 | 2.45633E−26 |
| Gpx4 | −0.351438364 | 10.07617554 | 121.6178872 | 2.79867E−28 | 2.77567E−26 |
| Rps23 | −0.138447821 | 12.48534445 | 121.4722896 | 3.01179E−28 | 2.96882E−26 |
| Gramd3 | 0.418596938 | 10.20637838 | 121.4465525 | 3.05111E−28 | 2.98935E−26 |
| Rps15 | −0.172864084 | 11.49080729 | 120.486991 | 4.94903E−28 | 4.81964E−26 |
| Gzmm | 0.434802686 | 9.16991855 | 118.6914094 | 1.22358E−27 | 1.18446E−25 |
| Icos | −0.390581255 | 10.25649242 | 118.6538485 | 1.24697E−27 | 1.19992E−25 |
| Dnaja1 | −0.411629472 | 10.76360037 | 118.4506794 | 1.38147E−27 | 1.32147E−25 |
| Plp2 | 0.532759958 | 9.268337634 | 117.7246556 | 1.99209E−27 | 1.89436E−25 |
| Cdc42 | 0.23089803 | 10.40482074 | 117.6820088 | 2.03538E−27 | 1.92421E−25 |
| Tppp3 | 0.695346253 | 9.186033366 | 117.5848399 | 2.13758E−27 | 2.00908E−25 |
| Tnfrsf1b | −0.53842263 | 9.452770816 | 117.4706506 | 2.26426E−27 | 2.11584E−25 |
| Capn2 | 0.485018692 | 9.322203269 | 114.5742255 | 9.75458E−27 | 9.06279E−25 |
| Eif3f | 0.203096883 | 10.66455686 | 114.1050524 | 1.23585E−26 | 1.14164E−24 |
| Lcp1 | 0.287302962 | 10.29112703 | 113.717244 | 1.5028E−26 | 1.38036E−24 |
| Stat1 | −0.513852248 | 9.580220981 | 113.5655779 | 1.62226E−26 | 1.48167E−24 |
| Jun | −0.94325618 | 9.603506659 | 113.0395558 | 2.11513E−26 | 1.92096E−24 |
| Tmsb4x | 0.144182849 | 14.24924733 | 112.5416481 | 2.71893E−26 | 2.45554E−24 |
| Clic1 | 0.268803753 | 10.32562522 | 112.5094932 | 2.76339E−26 | 2.48183E−24 |
| Vasp | 0.287636612 | 10.04115431 | 112.1944017 | 3.23938E−26 | 2.89325E−24 |
| Sema4a | 0.512251592 | 9.198626437 | 112.0481305 | 3.4874E−26 | 3.09766E−24 |
| Btf3 | 0.187828928 | 10.71559571 | 111.2147299 | 5.30965E−26 | 4.69048E−24 |
| Esyt1 | 0.475445557 | 9.338053167 | 110.1227206 | 9.21083E−26 | 8.09251E−24 |
| Arpc3 | −0.254844539 | 10.54018788 | 108.5503449 | 2.03612E−25 | 1.77924E−23 |
| Hsd11b1 | 0.527808948 | 9.289647124 | 107.9739699 | 2.72331E−25 | 2.36694E−23 |
| Rps25 | −0.179054311 | 11.11875547 | 107.5235213 | 3.41824E−25 | 2.95505E−23 |
| Areg | −1.179794443 | 9.38613304 | 107.4853962 | 3.48464E−25 | 2.99642E−23 |
| Rap1b | 0.265092354 | 10.1124176 | 107.3677512 | 3.69775E−25 | 3.16285E−23 |
| Tpt1 | −0.15517681 | 11.7834661 | 107.2473914 | 3.92927E−25 | 3.34319E−23 |
| Actr3 | 0.261765133 | 10.23426606 | 107.0671408 | 4.3034E−25 | 3.64235E−23 |
| Tox | −0.523743046 | 9.202083903 | 104.462925 | 1.60162E−24 | 1.34853E−22 |
| Cd7 | 0.758734546 | 9.494093275 | 104.186839 | 1.84109E−24 | 1.54213E−22 |
| Rpl23 | −0.156527601 | 11.70875625 | 103.6246787 | 2.44512E−24 | 2.03752E−22 |
| Supt4a | 0.284417019 | 9.962767148 | 103.560841 | 2.52519E−24 | 2.09345E−22 |
| Gm11808 | −0.317351685 | 10.17411983 | 101.4055452 | 7.49537E−24 | 6.18215E−22 |
| Rpl39 | −0.154446809 | 11.7773745 | 101.3066649 | 7.87902E−24 | 6.4656E−22 |
| Gna13 | −0.357361906 | 9.985128145 | 100.8831794 | 9.75716E−24 | 7.96638E−22 |
| Phf11b | 0.486488786 | 9.311766492 | 100.510537 | 1.17769E−23 | 9.56709E−22 |
| Eef1a1 | −0.130787248 | 12.38191171 | 99.99485692 | 1.52793E−23 | 1.23503E−21 |
| Hspa1a | −1.119432968 | 11.00098792 | 99.97583829 | 1.54268E−23 | 1.24074E−21 |
| Ccnd3 | 0.409152136 | 9.527402171 | 99.62411953 | 1.84247E−23 | 1.47452E−21 |
| Adam8 | 0.511454079 | 9.247167125 | 99.41606727 | 2.04655E−23 | 1.62978E−21 |
| Fosb | −0.690931948 | 9.410758224 | 99.12867187 | 2.36618E−23 | 1.87508E−21 |
| Prss16 | 0.453141756 | 9.187154617 | 99.03879299 | 2.47604E−23 | 1.95257E−21 |
| Fut7 | 0.243038107 | 9.160603564 | 98.82820564 | 2.75384E−23 | 2.1611E−21 |
| Capzb | 0.252231093 | 10.1589729 | 98.61753077 | 3.06296E−23 | 2.39207E−21 |
| 2810001G20Rik | 0.514611056 | 9.235596474 | 98.50063828 | 3.2492E−23 | 2.52532E−21 |
| Tra2b | −0.360766994 | 9.814709276 | 98.41122462 | 3.39927E−23 | 2.62931E−21 |
| Maf | −0.499169931 | 9.56864775 | 98.38318927 | 3.44774E−23 | 2.6541E−21 |
| Dnajc15 | 0.302461752 | 9.893147259 | 98.27310767 | 3.64482E−23 | 2.79252E−21 |
| Rpl9 | −0.13362575 | 11.84228944 | 96.87312552 | 7.39152E−23 | 5.63638E−21 |
| Dnajb1 | −0.701565277 | 10.3172835 | 96.68446985 | 8.13045E−23 | 6.17074E−21 |
| Sla | −0.448798408 | 9.546198944 | 96.60348318 | 8.46991E−23 | 6.39835E−21 |
| Ptprcap | 0.315090568 | 10.04014467 | 96.37240421 | 9.51847E−23 | 7.157E−21 |
| Nr4a2 | −0.567778459 | 9.521228879 | 96.12073243 | 1.08087E−22 | 8.08951E−21 |
| Gm9843 | −0.294369077 | 10.49166455 | 95.54204333 | 1.44784E−22 | 1.0786E−20 |
| Laptm5 | 0.208711783 | 10.49799071 | 94.88345685 | 2.0193E−22 | 1.49743E−20 |
| Snrpg | −0.264157695 | 10.10181353 | 94.14112586 | 2.93811E−22 | 2.16883E−20 |
| Ass1 | 0.437532946 | 9.53437863 | 92.9075629 | 5.47947E−22 | 4.02642E−20 |
| ler5l | −0.591031741 | 9.223274756 | 92.85145529 | 5.63704E−22 | 4.12345E−20 |
| Birc5 | −0.774093032 | 9.251668784 | 92.70648319 | 6.06546E−22 | 4.41685E−20 |
| Rpl21 | −0.141074021 | 11.76876712 | 92.34010486 | 7.29901E−22 | 5.29129E−20 |
| Lmnb1 | −0.395473345 | 9.781576618 | 92.00566036 | 8.64289E−22 | 6.23754E−20 |
| Qrfp | 0.327357379 | 9.165807799 | 91.69725749 | 1.01005E−21 | 7.25708E−20 |
| Eno1 | 0.29946273 | 9.942452612 | 91.61301618 | 1.05397E−21 | 7.53918E−20 |
| Ppp1ca | 0.233359723 | 10.29808235 | 91.22780891 | 1.28048E−21 | 9.11905E−20 |
| Gm10073 | −0.394855462 | 9.699374848 | 90.63658937 | 1.72638E−21 | 1.22407E−19 |
| Itpkb | −0.452002805 | 9.509585234 | 90.53161196 | 1.82045E−21 | 1.28513E−19 |
| Tmem176a | −0.709693164 | 9.404482276 | 90.2402785 | 2.10924E−21 | 1.48252E−19 |
| Mpnd | 0.436149431 | 9.307880167 | 90.19537586 | 2.15766E−21 | 1.50999E−19 |
| Rab8a | 0.368294453 | 9.516531936 | 89.88306722 | 2.5266E−21 | 1.76056E−19 |
| Mt1 | 0.685595236 | 9.253327294 | 89.15305586 | 3.65418E−21 | 2.53534E−19 |
| Capg | 0.383533992 | 9.809408711 | 88.6700908 | 4.66465E−21 | 3.2226E−19 |
| Ikzf2 | −0.697619136 | 9.29885187 | 88.40616184 | 5.33045E−21 | 3.6669E−19 |
| Hrh4 | 0.220443917 | 9.160771464 | 88.04349384 | 6.40309E−21 | 4.38612E−19 |
| Eif5 | −0.335801311 | 9.823445443 | 87.78758948 | 7.28748E−21 | 4.97086E−19 |
| Rpl41 | −0.105943424 | 12.79526034 | 87.62321264 | 7.91898E−21 | 5.37892E−19 |
| Degs1 | 0.387396398 | 9.418897111 | 87.48336602 | 8.49916E−21 | 5.74884E−19 |
| Ccna2 | −0.40044653 | 9.193824398 | 87.40535243 | 8.84109E−21 | 5.95521E−19 |
| Il4ra | −0.451028003 | 9.499550436 | 86.23410607 | 1.59847E−20 | 1.07223E−18 |
| Kcnab2 | 0.447184668 | 9.290281332 | 85.60058808 | 2.2021E−20 | 1.47104E−18 |
| Cdkn2d | 0.465467752 | 9.369239164 | 85.53454873 | 2.27688E−20 | 1.51474E−18 |
| Rps12-ps3 | −0.339355757 | 9.91463743 | 85.02653826 | 2.94387E−20 | 1.95044E−18 |
| Dok2 | 0.39150937 | 9.437395362 | 84.64612001 | 3.56843E−20 | 2.35458E−18 |
| Ecm1 | −0.527046667 | 9.213869135 | 84.63123432 | 3.5954E−20 | 2.36273E−18 |
| Srsf7 | −0.369743626 | 9.589293129 | 84.41319337 | 4.0146E−20 | 2.62753E−18 |
| Gnai2 | 0.258354443 | 10.17425826 | 84.02128898 | 4.89475E−20 | 3.19067E−18 |
| Atp5l | 0.187626855 | 10.47767657 | 82.95184884 | 8.40767E−20 | 5.45857E−18 |
| 2810474O19Rik | 0.414915888 | 9.518034833 | 82.51633462 | 1.048E−19 | 6.77682E−18 |
| Tmem176b | −0.636118826 | 9.448063427 | 82.22323647 | 1.21552E−19 | 7.82875E−18 |
| Cenpa | −0.530464482 | 9.394708498 | 80.8065773 | 2.48928E−19 | 1.59689E−17 |
| Rpl6l | −0.255942445 | 10.20631876 | 80.66181687 | 2.67848E−19 | 1.70474E−17 |
| Rplp0 | 0.119720189 | 12.38788281 | 80.66592463 | 2.67292E−19 | 1.70474E−17 |
| Srsf2 | −0.318215702 | 9.857765406 | 80.54991993 | 2.83453E−19 | 1.79698E−17 |
| Il1r2 | −0.709588642 | 9.25672179 | 80.41166541 | 3.03995E−19 | 1.91968E−17 |
| Cox4i1 | 0.165502192 | 10.68635697 | 79.34468754 | 5.21663E−19 | 3.2814E−17 |
| Sp100 | 0.35419291 | 9.553598448 | 79.04472727 | 6.07196E−19 | 3.80462E−17 |
| Coq10b | −0.402784111 | 9.509847044 | 78.97265199 | 6.29757E−19 | 3.93075E−17 |
| Lcn4 | 0.452058809 | 9.193732712 | 78.58412421 | 7.6663E−19 | 4.76667E−17 |
| Penk | −0.97476906 | 9.250330203 | 78.56769485 | 7.73033E−19 | 4.78807E−17 |
| Rps17 | −0.207724729 | 10.61521551 | 77.30519082 | 1.46481E−18 | 9.03819E−17 |
| Npc2 | 0.258897774 | 9.848502166 | 77.27191156 | 1.4897E−18 | 9.15682E−17 |
| Kpna1 | −0.465763262 | 9.374034121 | 77.23584317 | 1.51715E−18 | 9.29025E−17 |
| Klrc2 | 0.494282599 | 9.283377565 | 77.02917358 | 1.68451E−18 | 1.02762E−16 |
| 1810058124Rik | 0.350718585 | 9.463204041 | 76.79034685 | 1.90105E−18 | 1.15535E−16 |
| Tubb5 | −0.402918211 | 10.00329454 | 76.77948586 | 1.91153E−18 | 1.15737E−16 |
| Runx2 | 0.418744898 | 9.311867377 | 76.7116709 | 1.97831E−18 | 1.19333E−16 |
| Hspa1b | −0.811543031 | 9.330502334 | 76.31282443 | 2.42106E−18 | 1.45498E−16 |
| Pld3 | 0.396449863 | 9.363194614 | 76.05243089 | 2.76232E−18 | 1.65391E−16 |
| 2810417H13Rik | −1.011187289 | 9.337691412 | 75.408969 | 3.82648E−18 | 2.28261E−16 |
| Hist1h1c | 0.520548203 | 9.543347573 | 75.2055756 | 4.24168E−18 | 2.52099E−16 |
| Abi3 | −0.513763069 | 9.334628841 | 74.69953195 | 5.48091E−18 | 3.24558E−16 |
| Maff | −0.542704954 | 9.305194699 | 74.66275934 | 5.58396E−18 | 3.29453E−16 |
| Eprs | −0.467694903 | 9.384361092 | 74.62296026 | 5.69767E−18 | 3.3494E−16 |
| Plec | 0.418445279 | 9.207830137 | 74.5132662 | 6.02322E−18 | 3.52795E−16 |
| Ctsa | 0.36742707 | 9.447076929 | 74.12724047 | 7.32407E−18 | 4.2744E−16 |
| Uba52 | −0.247091632 | 10.23997211 | 73.81613397 | 8.57429E−18 | 4.98604E−16 |
| Xist | −0.668090773 | 9.888840859 | 73.76865447 | 8.78303E−18 | 5.08912E−16 |
| Stmn1 | −0.902189526 | 9.430632462 | 73.4820959 | 1.01553E−17 | 5.86324E−16 |
| Tmsb10 | 0.127806456 | 12.94248487 | 73.30106466 | 1.11308E−17 | 6.40357E−16 |
| Rps13 | −0.114822052 | 11.92527065 | 72.93365805 | 1.34082E−17 | 7.68641E−16 |
| Hmgn1 | −0.472844497 | 9.24964203 | 72.65748184 | 1.54221E−17 | 8.80968E−16 |
| Top2a | −0.571920918 | 9.285394761 | 72.45227309 | 1.71121E−17 | 9.74063E−16 |
| Spc24 | −0.4090324 | 9.204180171 | 72.24512376 | 1.9006E−17 | 1.07807E−15 |
| Ppp1r15a | −0.411070109 | 9.595245048 | 72.1100221 | 2.03527E−17 | 1.15043E−15 |
| Eomes | −0.680093092 | 9.215483506 | 71.67702638 | 2.53467E−17 | 1.42771E−15 |
| Swap70 | 0.410801492 | 9.194641096 | 71.595587 | 2.64147E−17 | 1.48271E−15 |
| Itga4 | 0.403875994 | 9.394391702 | 71.57584864 | 2.66802E−17 | 1.49243E−15 |
| Anp32e | −0.379405088 | 9.516990745 | 70.78954069 | 3.97439E−17 | 2.21551E−15 |
| Arl2bp | 0.321268348 | 9.473272776 | 70.77467715 | 4.00444E−17 | 2.2246E−15 |
| Sike1 | 0.404053142 | 9.285336783 | 70.63030965 | 4.30847E−17 | 2.3853E−15 |
| Myo1f | 0.413029505 | 9.304626444 | 70.51398558 | 4.57015E−17 | 2.52154E−15 |
| Fxyd4 | 0.216164291 | 9.161845803 | 70.34300404 | 4.98393E−17 | 2.74048E−15 |
| Insig1 | −0.468471973 | 9.373382406 | 70.3189397 | 5.0451E−17 | 2.76471E−15 |
| Anxa5 | 0.335340423 | 9.456501432 | 70.29264776 | 5.11279E−17 | 2.79234E−15 |
| Rps12 | −0.189372327 | 10.77087646 | 69.76259892 | 6.68892E−17 | 3.64085E−15 |
| Arpc2 | 0.155543095 | 10.79500611 | 69.53607194 | 7.50297E−17 | 4.07024E−15 |
| Pnrc1 | −0.22302859 | 10.57767668 | 69.46707965 | 7.77006E−17 | 4.20103E−15 |
| Ppib | 0.233842294 | 9.881020252 | 69.34980439 | 8.24608E−17 | 4.44354E−15 |
| Gm9493 | −0.228613016 | 10.33361801 | 69.2113078 | 8.84594E−17 | 4.75095E−15 |
| Dpcd | 0.422954441 | 9.248387156 | 69.18710349 | 8.95517E−17 | 4.79369E−15 |
| Rpl24 | 0.115597877 | 11.77230384 | 69.10660292 | 9.32825E−17 | 4.97692E−15 |
| Pkp3 | 0.322991067 | 9.70969078 | 69.06988323 | 9.50356E−17 | 5.05377E−15 |
| Crip2 | 0.493033563 | 9.219720142 | 68.71993548 | 1.13488E−16 | 6.01523E−15 |
| Rps6 | −0.104903549 | 12.20844385 | 68.69896404 | 1.14701E−16 | 6.05967E−15 |
| Cox5a | 0.222688552 | 10.08163797 | 68.41076629 | 1.32751E−16 | 6.99039E−15 |
| Kdm6b | −0.473433547 | 9.740181133 | 68.2056575 | 1.47303E−16 | 7.73147E−15 |
| Rbm3 | 0.190550874 | 10.73115847 | 67.86636293 | 1.74961E−16 | 9.15344E−15 |
| Rpl6 | −0.112569061 | 11.76088309 | 67.73571974 | 1.86946E−16 | 9.74893E−15 |
| Arpc4 | 0.248366607 | 9.810389381 | 67.10469229 | 2.57465E−16 | 1.33832E−14 |
| Rpl13 | −0.102585652 | 12.63773605 | 66.84961241 | 2.9303E−16 | 1.51346E−14 |
| D8Ertd738e | −0.24018408 | 10.02747279 | 66.85109585 | 2.92809E−16 | 1.51346E−14 |
| Hmgn2 | −0.499593273 | 9.445371749 | 66.69834967 | 3.164E−16 | 1.62896E−14 |
| Atox1 | 0.27625198 | 9.640004593 | 66.58567045 | 3.35013E−16 | 1.71931E−14 |
| Eef2 | 0.127179582 | 11.29644286 | 66.52871607 | 3.44833E−16 | 1.76411E−14 |
| Nr4a3 | −0.449594613 | 9.253600396 | 66.40569813 | 3.6704E−16 | 1.87179E−14 |
| H2afx | −0.484140494 | 9.351631242 | 66.38946902 | 3.70074E−16 | 1.88133E−14 |
| Rcbtb2 | −0.478710507 | 9.398952659 | 66.37266476 | 3.73242E−16 | 1.89149E−14 |
| Il17a | −0.932121997 | 9.237217593 | 66.09431516 | 4.29854E−16 | 2.17157E−14 |
| Tuba4a | −0.427821736 | 9.389392328 | 66.08179574 | 4.32593E−16 | 2.1786E−14 |
| Fau | −0.101663836 | 12.30166869 | 65.56169951 | 5.63228E−16 | 2.82768E−14 |
| Paxbp1 | −0.443740412 | 9.251233688 | 65.53032342 | 5.72266E−16 | 2.85533E−14 |
| Rpl5 | −0.176646222 | 10.56771244 | 65.53343568 | 5.71363E−16 | 2.85533E−14 |
| Rrm2 | −0.60210116 | 9.247674435 | 65.35593265 | 6.25214E−16 | 3.10991E−14 |
| Abcb1a | −0.448389508 | 9.300396921 | 65.34292109 | 6.29356E−16 | 3.12091E−14 |
| Pim3 | −0.482845753 | 9.328422325 | 65.07244979 | 7.21944E−16 | 3.5691E−14 |
| Spef2 | 0.337654887 | 9.175320605 | 65.03415325 | 7.36111E−16 | 3.62804E−14 |
| Car5b | 0.345768026 | 9.17782806 | 64.90035524 | 7.87829E−16 | 3.87114E−14 |
| Xrn2 | 0.37261421 | 9.343045417 | 64.8757557 | 7.97726E−16 | 3.90789E−14 |
| Ech1 | 0.305307325 | 9.539590664 | 64.52484105 | 9.5323E−16 | 4.65556E−14 |
| Rap1gap2 | 0.247182525 | 9.165395658 | 64.43601206 | 9.97188E−16 | 4.85558E−14 |
| Tox2 | −0.366384133 | 9.191330824 | 64.11910587 | 1.1712E−15 | 5.68578E−14 |
| Tnfaip8l2 | 0.393193127 | 9.325796745 | 63.9932113 | 1.24849E−15 | 6.04283E−14 |
| Nt5c | 0.321207331 | 9.46177492 | 63.91994481 | 1.29579E−15 | 6.25306E−14 |
| Rasgrp1 | −0.390344872 | 9.487650699 | 63.86570537 | 1.33196E−15 | 6.40848E−14 |
| Prelid1 | 0.277620155 | 9.693827343 | 63.70800001 | 1.44297E−15 | 6.92199E−14 |
| Rpl13a | 0.093506977 | 13.1315227 | 63.41756016 | 1.67221E−15 | 7.9979E−14 |
| Cks1b | −0.54776796 | 9.261766706 | 63.35897481 | 1.72269E−15 | 8.21503E−14 |
| Mmd | −0.493783446 | 9.302850291 | 63.28725567 | 1.78656E−15 | 8.49459E−14 |
| Serpini1 | 0.297936677 | 9.172144254 | 63.1533642 | 1.91223E−15 | 9.06541E−14 |
| Rps18-ps3 | −0.383942395 | 9.484983983 | 63.13228605 | 1.9328E−15 | 9.13615E−14 |
| Atp8b4 | 0.282378055 | 9.170105411 | 62.70317375 | 2.40328E−15 | 1.13269E−13 |
| Chchd2 | −0.185649555 | 10.44214805 | 62.67240577 | 2.44112E−15 | 1.14718E−13 |
| Rpl22 | −0.161149578 | 10.60718396 | 62.52082164 | 2.63643E−15 | 1.23538E−13 |
| Madd | 0.355724386 | 9.387289733 | 62.16934741 | 3.15155E−15 | 1.47249E−13 |
| Spc25 | −0.265138721 | 9.180366242 | 62.11597042 | 3.23815E−15 | 1.50858E−13 |
| Apobec3 | 0.340744453 | 9.503283445 | 61.75805314 | 3.8836E−15 | 1.80409E−13 |
| Nmrk1 | −0.43682095 | 9.470654245 | 61.62438468 | 4.15639E−15 | 1.92528E−13 |
| Ltb | 0.299299727 | 10.15492701 | 61.50031252 | 4.42673E−15 | 2.04464E−13 |
| Itgb2 | 0.302934438 | 9.575070658 | 61.4814308 | 4.46938E−15 | 2.05846E−13 |
| Rgs19 | 0.371295223 | 9.28716881 | 61.40647112 | 4.64282E−15 | 2.13227E−13 |
| Chchd10 | −0.473427777 | 9.239735277 | 61.29318067 | 4.91781E−15 | 2.25216E−13 |
| Arhgap15 | 0.327112956 | 9.436551627 | 60.90418079 | 5.99215E−15 | 2.73641E−13 |
| 5430427O19Rik | 0.372356642 | 9.195518327 | 60.83083196 | 6.21962E−15 | 2.83229E−13 |
| 1110008F13Rik | 0.315751896 | 9.430011424 | 60.72906256 | 6.5496E−15 | 2.97418E−13 |
| Adora2a | −0.392567249 | 9.426697678 | 60.21879694 | 8.48778E−15 | 3.84351E−13 |
| Cbx3 | −0.405488348 | 9.517985802 | 60.17684028 | 8.67064E−15 | 3.91535E−13 |
| Snx10 | 0.373362566 | 9.204288337 | 60.06212372 | 9.19101E−15 | 4.13877E−13 |
| Ccr4 | −0.421655714 | 9.227421818 | 59.72211968 | 1.09242E−14 | 4.90556E−13 |
| Cxcl10 | −0.664443182 | 9.358371286 | 59.64847266 | 1.13407E−14 | 5.07851E−13 |
| Cfl1 | 0.149724783 | 11.11290366 | 59.32651689 | 1.33565E−14 | 5.96252E−13 |
| Fam65b | 0.396364559 | 9.256723776 | 59.32179213 | 1.33886E−14 | 5.96252E−13 |
| Kxd1 | 0.3361848 | 9.397333347 | 59.2482617 | 1.38983E−14 | 6.17253E−13 |
| Nusap1 | −0.426549788 | 9.207238975 | 59.13228173 | 1.47421E−14 | 6.52933E−13 |
| P2ry14 | 0.347177821 | 9.17900749 | 59.03492232 | 1.54898E−14 | 6.84177E−13 |
| Oat | −0.395947403 | 9.329291217 | 58.93175541 | 1.63236E−14 | 7.19041E−13 |
| Flt3l | 0.35959273 | 9.333363234 | 58.9039048 | 1.65563E−14 | 7.27309E−13 |
| Fmnl1 | 0.350065739 | 9.349669898 | 58.68899791 | 1.84671E−14 | 8.0905E−13 |
| Morf412 | −0.364938214 | 9.397534901 | 58.42868198 | 2.10795E−14 | 9.21005E−13 |
| Slc25a24 | 0.377308788 | 9.218974536 | 58.36398837 | 2.17842E−14 | 9.49228E−13 |
| Rpl14 | −0.107475124 | 11.87708218 | 58.29747299 | 2.25333E−14 | 9.79231E−13 |
| Faah | 0.370597532 | 9.275976683 | 58.27112054 | 2.28372E−14 | 9.89775E−13 |
| Cotl1 | 0.197250984 | 10.46134647 | 58.24188048 | 2.31792E−14 | 9.99238E−13 |
| Tmem71 | 0.38204988 | 9.273813725 | 58.24380723 | 2.31565E−14 | 9.99238E−13 |
| Plek | 0.43842105 | 9.215818477 | 57.95124621 | 2.68695E−14 | 1.15525E−12 |
| Fam110a | −0.37843848 | 9.494240477 | 57.91214141 | 2.7409E−14 | 1.17532E−12 |
| Calm2 | −0.204357886 | 10.41345274 | 57.66217283 | 3.11231E−14 | 1.33105E−12 |
| Cdc20 | −0.352635513 | 9.190015265 | 57.57131431 | 3.25945E−14 | 1.3903E−12 |
| Rilpl2 | 0.353546322 | 9.398891472 | 57.47613338 | 3.42105E−14 | 1.45539E−12 |
| Nucks1 | −0.385021534 | 9.378610594 | 57.24063171 | 3.85622E−14 | 1.63621E−12 |
| Traf4 | −0.412707476 | 9.35979853 | 57.1752643 | 3.98654E−14 | 1.68708E−12 |
| Myo1g | 0.326411976 | 9.439455324 | 56.91402368 | 4.55288E−14 | 1.92172E−12 |
| PISD | −0.424075103 | 9.326147814 | 56.80816092 | 4.80468E−14 | 2.02272E−12 |
| Bbc3 | 0.390523027 | 9.210384865 | 56.78266578 | 4.86738E−14 | 2.04379E−12 |
| Mki67 | −0.367343453 | 9.203057109 | 56.75183508 | 4.94429E−14 | 2.07071E−12 |
| 1700025G04Rik | 0.355444841 | 9.18123029 | 56.74235589 | 4.96818E−14 | 2.07534E−12 |
| Phlda1 | −0.491778784 | 9.440764624 | 56.55498898 | 5.46486E−14 | 2.27693E−12 |
| Tmem160 | 0.265315817 | 9.59150512 | 56.52865987 | 5.53852E−14 | 2.30169E−12 |
| Arf5 | 0.198635135 | 9.981768048 | 56.30803437 | 6.19617E−14 | 2.56839E−12 |
| Trpv2 | 0.372377047 | 9.250925444 | 56.10050974 | 6.88593E−14 | 2.847E−12 |
| Usp3 | 0.355354175 | 9.292631844 | 56.06888598 | 6.99758E−14 | 2.88579E−12 |
| Pqlc3 | 0.362365955 | 9.234748342 | 56.01745486 | 7.18305E−14 | 2.95474E−12 |
| Rpl9-ps6 | −0.290907311 | 9.841070067 | 55.92224168 | 7.53948E−14 | 3.09348E−12 |
| Gabpb1 | −0.386813549 | 9.478096364 | 55.7462451 | 8.24556E−14 | 3.37463E−12 |
| Rpl37 | −0.100831831 | 12.07305382 | 55.51209471 | 9.28858E−14 | 3.7919E−12 |
| Ptprc | 0.203113685 | 10.11524395 | 55.28566724 | 1.04226E−13 | 4.24412E−12 |
| Tmem234 | 0.252630089 | 9.671898221 | 55.08446668 | 1.1546E−13 | 4.68976E−12 |
| Tesc | −0.34262081 | 9.483468613 | 55.07619352 | 1.15947E−13 | 4.69773E−12 |
| Xcl1 | −0.961147086 | 9.405065931 | 55.04662835 | 1.17704E−13 | 4.75701E−12 |
| Lag3 | −0.642931722 | 9.272156156 | 55.01140439 | 1.19832E−13 | 4.83095E−12 |
| Uqcrfs1 | 0.229745717 | 9.714864078 | 54.99578919 | 1.20788E−13 | 4.85737E−12 |
| Tktl1 | 0.186029244 | 9.161740461 | 54.91081279 | 1.26125E−13 | 5.0594E−12 |
| Edf1 | 0.203844061 | 9.905022216 | 54.87068451 | 1.28727E−13 | 5.15099E−12 |
| Cirbp | −0.263662128 | 9.821626437 | 54.83975146 | 1.30769E−13 | 5.21978E−12 |
| Ska1 | −0.153998658 | 9.167697937 | 54.81765797 | 1.32247E−13 | 5.26578E−12 |
| Cdc42se1 | 0.329661747 | 9.377124053 | 54.67523687 | 1.42187E−13 | 5.64764E−12 |
| Asf1b | −0.438345195 | 9.223299882 | 54.65077324 | 1.43968E−13 | 5.70437E−12 |
| Hmgb1 | −0.219889837 | 10.29714889 | 54.58969676 | 1.48512E−13 | 5.87005E−12 |
| Hic1 | −0.432264919 | 9.270363669 | 54.4810714 | 1.56952E−13 | 6.18852E−12 |
| Sept11 | 0.356045397 | 9.343747121 | 54.27766493 | 1.74069E−13 | 6.84672E−12 |
| Tprgl | 0.251967719 | 9.655126678 | 54.16606559 | 1.84241E−13 | 7.22924E−12 |
| Nfatc2 | −0.402648226 | 9.26546375 | 54.15455217 | 1.85324E−13 | 7.25411E−12 |
| Gyg | 0.370089737 | 9.256694676 | 54.09174115 | 1.91344E−13 | 7.47165E−12 |
| H2-K1 | −0.13757215 | 11.61002041 | 53.98001915 | 2.02539E−13 | 7.88974E−12 |
| Isy1 | −0.370234123 | 9.388840432 | 53.94586091 | 2.06091E−13 | 8.0088E−12 |
| Cenpe | −0.220150784 | 9.175332099 | 53.84735181 | 2.16687E−13 | 8.38029E−12 |
| Fryl | −0.393078983 | 9.288378553 | 53.84761229 | 2.16658E−13 | 8.38029E−12 |
| Tacc3 | −0.302069013 | 9.190084756 | 53.74794908 | 2.27932E−13 | 8.79415E−12 |
| Dusp4 | −0.416920504 | 9.244696816 | 53.55668266 | 2.51238E−13 | 9.67028E−12 |
| Tpm4 | 0.30198206 | 9.580032755 | 53.4530001 | 2.64854E−13 | 1.01701E−11 |
| Ccdc12 | 0.24770037 | 9.642269047 | 53.43241053 | 2.67645E−13 | 1.02529E−11 |
| Stk11 | −0.342997764 | 9.400849509 | 53.3637069 | 2.77171E−13 | 1.05928E−11 |
| Acp5 | 0.327797466 | 9.39691335 | 53.27003737 | 2.90707E−13 | 1.10839E−11 |
| Serf2 | 0.134205382 | 10.706856 | 53.12999948 | 3.12188E−13 | 1.18749E−11 |
| Nr3c1 | −0.376848168 | 9.448729303 | 52.68065901 | 3.92437E−13 | 1.48923E−11 |
| Cdca8 | −0.387769623 | 9.207959893 | 52.53102797 | 4.23504E−13 | 1.60337E−11 |
| Srrm2 | −0.257041817 | 9.807993986 | 52.48496978 | 4.33554E−13 | 1.63758E−11 |
| Prkcq | 0.358224651 | 9.276198391 | 52.33872288 | 4.67073E−13 | 1.76007E−11 |
| Ccl4 | −1.176767063 | 9.567226392 | 52.26773318 | 4.84267E−13 | 1.82062E−11 |
| Smc2 | −0.437413231 | 9.240676881 | 52.25511877 | 4.87387E−13 | 1.8281E−11 |
| Kcnn4 | 0.360084555 | 9.328517363 | 52.18835536 | 5.04243E−13 | 1.88694E−11 |
| Icam1 | 0.396186315 | 9.404345015 | 51.59667848 | 6.81568E−13 | 2.54463E−11 |
| Tpgs1 | 0.358707501 | 9.260754154 | 51.49756628 | 7.16859E−13 | 2.67022E−11 |
| Lck | 0.169753726 | 10.31649673 | 51.26743412 | 8.06017E−13 | 2.99542E−11 |
| Riok1 | −0.390179495 | 9.379968099 | 51.08907685 | 8.82681E−13 | 3.25786E−11 |
| Mapkapk3 | 0.323418904 | 9.378101749 | 51.09610669 | 8.79526E−13 | 3.25786E−11 |
| Dnajb2 | −0.356805427 | 9.204584167 | 51.09030293 | 8.8213E−13 | 3.25786E−11 |
| Rrm1 | −0.36213335 | 9.219702428 | 51.03600203 | 9.06873E−13 | 3.33952E−11 |
| Ckb | −0.422149772 | 9.335719986 | 50.72744307 | 1.06125E−12 | 3.89914E−11 |
| Rpl13-ps3 | −0.279124517 | 9.66187407 | 50.69891137 | 1.07679E−12 | 3.94726E−11 |
| Lin54 | −0.392624993 | 9.319364611 | 50.60318457 | 1.13061E−12 | 4.13518E−11 |
| Aurkb | −0.208001106 | 9.176325027 | 50.50706228 | 1.18737E−12 | 4.33295E−11 |
| Fam71b | 0.31531926 | 9.185043177 | 50.47467455 | 1.20713E−12 | 4.39513E−11 |
| Fam188a | 0.352368275 | 9.234514724 | 50.44674626 | 1.22443E−12 | 4.44811E−11 |
| Cyb5r4 | 0.316676256 | 9.336417497 | 50.28053448 | 1.33265E−12 | 4.8304E−11 |
| Rnf138 | 0.269807224 | 9.608877256 | 50.16919159 | 1.41045E−12 | 5.10097E−11 |
| H2-Q4 | −0.265803125 | 9.753674988 | 50.11945341 | 1.44666E−12 | 5.22024E−11 |
| Gpr171 | 0.266612793 | 9.722723261 | 50.08308592 | 1.47372E−12 | 5.30604E−11 |
| mt-Nd1 | −0.157466526 | 11.14928249 | 49.66729327 | 1.82157E−12 | 6.5439E−11 |
| Arhgap30 | 0.318944329 | 9.36964608 | 49.49433071 | 1.98946E−12 | 7.13117E−11 |
| Ranbp1 | −0.295883166 | 9.626989183 | 49.47831386 | 2.00577E−12 | 7.17194E−11 |
| Ube2d3 | 0.142474457 | 10.46165668 | 49.47446563 | 2.00971E−12 | 7.17194E−11 |
| Gng2 | 0.272653394 | 9.504051907 | 49.4405448 | 2.04476E−12 | 7.28096E−11 |
| Gm7676 | 0.177214754 | 9.1626667 | 49.12506242 | 2.40152E−12 | 8.53253E−11 |
| Rps18 | −0.093749751 | 12.47336152 | 49.06724846 | 2.47336E−12 | 8.76848E−11 |
| Atp5c1 | 0.195509129 | 9.901795293 | 48.70713718 | 2.97183E−12 | 1.05126E−10 |
| Rpl31 | −0.139099182 | 10.87169602 | 48.69410038 | 2.99165E−12 | 1.05596E−10 |
| Tnfaip3 | −0.241621431 | 10.12478557 | 48.62414786 | 3.10028E−12 | 1.09192E−10 |
| Mrpl52 | −0.256915947 | 9.675785023 | 48.59779994 | 3.14222E−12 | 1.10428E−10 |
| Csrnp1 | −0.361872414 | 9.467384752 | 48.40889259 | 3.45996E−12 | 1.21331E−10 |
| Nudc | −0.299772679 | 9.486198177 | 48.36831434 | 3.5323E−12 | 1.236E−10 |
| Gdi2 | −0.219013875 | 9.887687816 | 48.28408552 | 3.68733E−12 | 1.28468E−10 |
| Hnrnpab | −0.306971441 | 9.483082447 | 48.28732967 | 3.68123E−12 | 1.28468E−10 |
| H2afz | −0.227597078 | 10.76601923 | 48.17724714 | 3.8938E−12 | 1.3537E−10 |
| Lef1 | 0.357453344 | 9.289398985 | 48.07662452 | 4.09884E−12 | 1.42193E−10 |
| Sgip1 | −0.382056564 | 9.193478703 | 47.83666323 | 4.63248E−12 | 1.60361E−10 |
| Wnk1 | −0.291417518 | 9.627909802 | 47.80634854 | 4.70466E−12 | 1.62512E−10 |
| Aldoa | 0.180711042 | 10.23632336 | 47.80033381 | 4.71912E−12 | 1.62664E−10 |
| Dusp 14 | −0.452661472 | 9.24162358 | 47.66873348 | 5.04677E−12 | 1.73587E−10 |
| Rpl27-ps3 | −0.279625551 | 9.836541137 | 47.62586986 | 5.15833E−12 | 1.77048E−10 |
| Sema4d | 0.313025469 | 9.35787998 | 47.5753698 | 5.29293E−12 | 1.81283E−10 |
| Nfkbid | −0.465977677 | 9.347258824 | 47.55442849 | 5.34978E−12 | 1.82842E−10 |
| Ccr7 | −0.653811338 | 9.273164068 | 47.46702965 | 5.59369E−12 | 1.90775E−10 |
| Napsa | 0.299633138 | 9.173963637 | 47.45450146 | 5.62955E−12 | 1.91594E−10 |
| Mt2 | 0.405205255 | 9.179003522 | 47.44257025 | 5.66392E−12 | 1.92359E−10 |
| Impdh2 | −0.328858199 | 9.377823225 | 47.41545958 | 5.7428E−12 | 1.94629E−10 |
| Tpx2 | −0.235310258 | 9.180975529 | 47.31848587 | 6.03405E−12 | 2.04072E−10 |
| D930028M14Rik | 0.221228249 | 9.165073917 | 47.30826359 | 6.0656E−12 | 2.04711E−10 |
| Shcbp1 | −0.186670641 | 9.173032321 | 47.12435823 | 6.66226E−12 | 2.24379E−10 |
| Snx5 | 0.285118196 | 9.443629607 | 46.87702366 | 7.55837E−12 | 2.5403E−10 |
| Rnf13 | 0.334006595 | 9.270089812 | 46.83603919 | 7.7181E−12 | 2.5886E−10 |
| Zfp36l1 | −0.354382619 | 9.536461336 | 46.67959216 | 8.3595E−12 | 2.79792E−10 |
| Mad2l1 | −0.336041583 | 9.208096533 | 46.66245921 | 8.43291E−12 | 2.81666E−10 |
| Gm8369 | 0.245202324 | 9.924470839 | 46.62050658 | 8.61539E−12 | 2.87168E−10 |
| Itgb1bp1 | 0.312257232 | 9.308749164 | 46.50417327 | 9.14235E−12 | 3.04106E−10 |
| Npm3 | 0.254196025 | 9.590225344 | 46.44713082 | 9.41241E−12 | 3.11805E−10 |
| Slamf7 | 0.355858539 | 9.281091881 | 46.4475631 | 9.41033E−12 | 3.11805E−10 |
| Nop10 | −0.247091186 | 9.680893727 | 46.36563341 | 9.81215E−12 | 3.24383E−10 |
| IlZrb | 0.250143282 | 9.646387369 | 46.35369914 | 9.8721E−12 | 3.25699E−10 |
| St13 | −0.295016503 | 9.483628577 | 46.29117192 | 1.01922E−11 | 3.35575E−10 |
| Lamb3 | 0.352261752 | 9.282524225 | 46.10335197 | 1.12176E−11 | 3.68586E−10 |
| 4930503L19Rik | −0.366113571 | 9.217222488 | 46.0856953 | 1.13192E−11 | 3.71168E−10 |
| Psmb3 | 0.188881553 | 9.906083726 | 46.05925169 | 1.1473E−11 | 3.75451E−10 |
| Itm2c | −0.297908775 | 9.518082662 | 45.91218367 | 1.23674E−11 | 4.03903E−10 |
| Dynll2 | −0.337577901 | 9.353846602 | 45.64014248 | 1.42099E−11 | 4.63141E−10 |
| Ppp3ca | 0.310612578 | 9.326663671 | 45.56895099 | 1.47359E−11 | 4.79317E−10 |
| Pdcd10 | 0.238398558 | 9.559474431 | 45.50441543 | 1.52295E−11 | 4.94378E−10 |
| Hnrnpa1 | −0.26197532 | 9.626210649 | 45.43016426 | 1.58179E−11 | 5.12451E−10 |
| Cxcr6 | 0.246872073 | 10.38993842 | 45.24422742 | 1.73932E−11 | 5.62358E−10 |
| Ckap2l | −0.110672662 | 9.164094745 | 45.23933167 | 1.74368E−11 | 5.62641E−10 |
| Ndufa5 | −0.282769786 | 9.479890673 | 44.84834027 | 2.12901E−11 | 6.8561E−10 |
| Pbk | −0.121046883 | 9.166098176 | 44.70325909 | 2.29275E−11 | 7.36872E−10 |
| Slc3a2 | −0.220945351 | 10.08964541 | 44.59582917 | 2.42207E−11 | 7.76889E−10 |
| Rbms1 | 0.285512041 | 9.408009945 | 44.5829661 | 2.43804E−11 | 7.80461E−10 |
| Tbc1d10c | 0.280613085 | 9.522585953 | 44.55291444 | 2.47575E−11 | 7.90967E−10 |
| Tiparp | −0.383475799 | 9.253515787 | 44.52403539 | 2.51254E−11 | 8.01137E−10 |
| Ablim1 | 0.286261251 | 9.407304211 | 44.49848449 | 2.54554E−11 | 8.10064E−10 |
| Ccnb1 | −0.246088895 | 9.183063921 | 44.26867125 | 2.86261E−11 | 9.09174E−10 |
| Fyb | −0.19056904 | 10.08185682 | 44.23904901 | 2.90626E−11 | 9.21226E−10 |
| Smco4 | −0.348268101 | 9.324729592 | 44.19116783 | 2.97822E−11 | 9.4219E−10 |
| Nhp2l1 | −0.22781492 | 9.714965176 | 43.83830396 | 3.56656E−11 | 1.12611E−09 |
| Ncl | −0.191524875 | 10.07286917 | 43.70541544 | 3.81714E−11 | 1.20288E−09 |
| Cldnd1 | −0.308788378 | 9.795081241 | 43.55524172 | 4.12159E−11 | 1.2963E−09 |
| Marcksl1 | −0.412529974 | 9.352502046 | 43.54726008 | 4.13843E−11 | 1.29907E−09 |
| Hist1h2ap | −0.268260711 | 9.186664241 | 43.4233744 | 4.4089E−11 | 1.38128E−09 |
| Avl9 | −0.326052978 | 9.389562788 | 43.36184802 | 4.54973E−11 | 1.42265E−09 |
| Itgav | −0.360644343 | 9.245488736 | 43.33825691 | 4.60492E−11 | 1.43712E−09 |
| Hk2 | −0.407653809 | 9.291524998 | 43.16055459 | 5.04271E−11 | 1.57072E−09 |
| AW011738 | −0.339602901 | 9.202092298 | 43.07369562 | 5.27163E−11 | 1.63887E−09 |
| Ppp1cc | −0.199608403 | 10.14033253 | 42.81442413 | 6.01867E−11 | 1.86752E−09 |
| Bpnt1 | −0.336240301 | 9.20504925 | 42.69801734 | 6.3877E−11 | 1.97823E−09 |
| Mvb12a | 0.294858646 | 9.323824234 | 42.65192056 | 6.54002E−11 | 2.02153E−09 |
| Eif3h | 0.142723574 | 10.29701565 | 42.48500485 | 7.12262E−11 | 2.19741E−09 |
| Psmb1 | 0.150510975 | 10.1666932 | 42.38821715 | 7.48396E−11 | 2.30449E−09 |
| Acot7 | 0.241728025 | 9.577204572 | 42.3784288 | 7.5215E−11 | 2.31165E−09 |
| Bak1 | 0.314497757 | 9.291916907 | 42.22934344 | 8.11729E−11 | 2.49002E−09 |
| Grap | 0.253931168 | 9.547624225 | 42.1727058 | 8.35581E−11 | 2.55833E−09 |
| Tpst2 | 0.257626354 | 9.502673289 | 42.15008125 | 8.45304E−11 | 2.58321E−09 |
| Nuf2 | −0.124493218 | 9.166373674 | 42.01178275 | 9.07251E−11 | 2.76729E−09 |
| Card19 | 0.31293595 | 9.298133437 | 42.00070748 | 9.12404E−11 | 2.77776E−09 |
| Nop56 | −0.297251616 | 9.449988788 | 41.996255 | 9.14484E−11 | 2.77886E−09 |
| Pdlim1 | 0.34171296 | 9.286551213 | 41.96937305 | 9.27143E−11 | 2.81204E−09 |
| St3gal4 | 0.317411314 | 9.222969771 | 41.85826443 | 9.81351E−11 | 2.97088E−09 |
| Fcgrt | 0.184065277 | 9.164513649 | 41.8039417 | 1.009E−10 | 3.04887E−09 |
| Msrb1 | 0.314774706 | 9.280628983 | 41.77610517 | 1.02347E−10 | 3.08682E−09 |
| Rasgrp4 | −0.295228004 | 9.196046455 | 41.7685978 | 1.0274E−10 | 3.09292E−09 |
| AI467606 | 0.324438402 | 9.267747664 | 41.73680495 | 1.04425E−10 | 3.13779E−09 |
| Cdc37 | 0.203897376 | 9.718870186 | 41.71248815 | 1.05731E−10 | 3.17116E−09 |
| Casc5 | −0.148113275 | 9.168214058 | 41.66042957 | 1.08585E−10 | 3.2507E−09 |
| Plk1 | −0.149116992 | 9.169178309 | 41.61760036 | 1.1099E−10 | 3.31656E−09 |
| Tspan13 | −0.27748507 | 9.546665786 | 41.59956108 | 1.12018E−10 | 3.34112E−09 |
| Lrrfip1 | 0.242872199 | 9.612103437 | 41.52173694 | 1.16568E−10 | 3.47041E−09 |
| Phpt1 | −0.340431832 | 9.254999299 | 41.51672903 | 1.16867E−10 | 3.47291E−09 |
| Neurl3 | −0.286535533 | 9.619611619 | 41.47993197 | 1.19087E−10 | 3.53241E−09 |
| Samsn1 | −0.284615787 | 9.795489494 | 41.41464949 | 1.23131E−10 | 3.64568E−09 |
| Zfand2b | 0.305793679 | 9.278292325 | 41.37076738 | 1.25927E−10 | 3.72162E−09 |
| Myl12a | 0.136720366 | 10.6785009 | 41.28722683 | 1.31425E−10 | 3.87703E−09 |
| Abhd17c | −0.333647132 | 9.339492108 | 41.13540964 | 1.4204E−10 | 4.18254E−09 |
| H1f0 | 0.390304588 | 9.42979381 | 41.10570101 | 1.44215E−10 | 4.23887E−09 |
| Eif4e3 | 0.324534257 | 9.248327932 | 41.0449197 | 1.4877E−10 | 4.36474E−09 |
| Glmp | 0.274250566 | 9.373701727 | 41.04141669 | 1.49037E−10 | 4.36474E−09 |
| Rps3a1 | −0.090667436 | 11.67400244 | 40.92375183 | 1.58286E−10 | 4.62721E−09 |
| Gm15518 | 0.228681393 | 9.168202192 | 40.85271619 | 1.64145E−10 | 4.78983E−09 |
| Rpl15 | −0.128287104 | 10.73595635 | 40.74537027 | 1.73414E−10 | 5.05118E−09 |
| AI662270 | 0.246465493 | 9.519765054 | 40.71742886 | 1.75911E−10 | 5.11471E−09 |
| Mettl23 | 0.24517514 | 9.492427646 | 40.62567419 | 1.84368E−10 | 5.35098E−09 |
| Klhdc2 | 0.319366014 | 9.222186834 | 40.59634857 | 1.87156E−10 | 5.42216E−09 |
| Mina | 0.323015776 | 9.23713881 | 40.58692412 | 1.88061E−10 | 5.43863E−09 |
| Tspan17 | 0.219617857 | 9.166558893 | 40.57116773 | 1.89584E−10 | 5.47287E−09 |
| Mast4 | −0.268505285 | 9.181693356 | 40.49239029 | 1.97383E−10 | 5.68788E−09 |
| Nrros | 0.326643367 | 9.212274046 | 40.45712311 | 2.00978E−10 | 5.78117E−09 |
| Rpl35a | −0.091306405 | 11.72350894 | 40.31639133 | 2.15989E−10 | 6.16905E−09 |
| Tecpr1 | 0.291847632 | 9.413080476 | 40.31802823 | 2.15808E−10 | 6.16905E−09 |
| Tnfaip8 | −0.272987616 | 9.630271166 | 40.31930658 | 2.15667E−10 | 6.16905E−09 |
| Iqgap2 | 0.31845389 | 9.250835755 | 40.32196987 | 2.15373E−10 | 6.16905E−09 |
| Nacc2 | 0.113924114 | 9.158821816 | 40.16272262 | 2.33665E−10 | 6.66212E−09 |
| Sdc4 | −0.319690858 | 9.184319545 | 40.14933305 | 2.35272E−10 | 6.69613E−09 |
| Cox5b | 0.173428491 | 10.04348938 | 40.0573221 | 2.46619E−10 | 7.00674E−09 |
| Clec12a | 0.347851947 | 9.187024502 | 39.98950738 | 2.55331E−10 | 7.24154E−09 |
| Gadd45a | −0.316811725 | 9.199789132 | 39.9749406 | 2.57242E−10 | 7.28297E−09 |
| Osbpl9 | −0.347920433 | 9.306529663 | 39.95290108 | 2.60161E−10 | 7.35273E−09 |
| Fabp5 | −0.345368512 | 9.261083113 | 39.92194667 | 2.64317E−10 | 7.45714E−09 |
| Skil | −0.300077713 | 9.558668519 | 39.91051627 | 2.65868E−10 | 7.48784E−09 |
| Wdr95 | 0.256187953 | 9.168701519 | 39.61982647 | 3.08535E−10 | 8.6744E−09 |
| Plac8 | 0.315422604 | 10.56912296 | 39.54916829 | 3.19903E−10 | 8.97838E−09 |
| Gzmb | 0.462504549 | 10.35039813 | 39.53373005 | 3.22442E−10 | 9.03395E−09 |
| Uqcrh | 0.128854127 | 10.54582243 | 39.49939278 | 3.28161E−10 | 9.1783E−09 |
| Uqcrc2 | 0.243771762 | 9.469343526 | 39.41541302 | 3.42582E−10 | 9.56508E−09 |
| Cdk1 | −0.282404204 | 9.197337951 | 39.40808657 | 3.4387E−10 | 9.58449E−09 |
| Gm10263 | −0.356543926 | 9.257269073 | 39.30048844 | 3.6335E−10 | 1.011E−08 |
| Jak2 | −0.339854318 | 9.275558522 | 39.22581003 | 3.77517E−10 | 1.04861E−08 |
| Tff1 | −0.343877655 | 9.186730263 | 39.16799524 | 3.88863E−10 | 1.07828E−08 |
| Lypla1 | 0.281543012 | 9.34948844 | 39.03404604 | 4.16479E−10 | 1.15288E−08 |
| H2afv | −0.270944916 | 9.565009799 | 38.95620237 | 4.33421E−10 | 1.19772E−08 |
| Cmtm7 | 0.271258758 | 9.51328408 | 38.81166005 | 4.66732E−10 | 1.28757E−08 |
| Zdhhc20 | 0.287294355 | 9.3491135 | 38.69079567 | 4.96545E−10 | 1.36293E−08 |
| Sla2 | 0.304926143 | 9.310517201 | 38.69603223 | 4.95215E−10 | 1.36293E−08 |
| Kif4 | −0.083959125 | 9.162292079 | 38.69067267 | 4.96576E−10 | 1.36293E−08 |
| Ythdc1 | −0.304345812 | 9.376355755 | 38.65093522 | 5.0679E−10 | 1.3886E−08 |
| Nek2 | −0.092225985 | 9.163012699 | 38.61679108 | 5.15733E−10 | 1.41072E−08 |
| Rad51 | −0.174396222 | 9.174504007 | 38.53074142 | 5.3898E−10 | 1.47182E−08 |
| Serp2 | 0.23272394 | 9.169810674 | 38.36728567 | 5.86067E−10 | 1.5977E−08 |
| Dstn | 0.315401763 | 9.262001726 | 38.26911479 | 6.16304E−10 | 1.6773E−08 |
| Stk17b | 0.177968741 | 10.0837855 | 38.26279885 | 6.18302E−10 | 1.67991E−08 |
| Rps7 | −0.087706789 | 11.94320961 | 38.23534949 | 6.27061E−10 | 1.70085E−08 |
| Rad51ap1 | −0.140603093 | 9.168896905 | 38.1844517 | 6.43632E−10 | 1.74287E−08 |
| 1700097N02Rik | 0.369642879 | 9.22738468 | 38.17693316 | 6.46117E−10 | 1.74668E−08 |
| Gbp9 | −0.314504429 | 9.2142636 | 38.1724911 | 6.4759E−10 | 1.74774E−08 |
| Ppdpf | 0.258511912 | 9.446285275 | 38.05634714 | 6.87308E−10 | 1.85184E−08 |
| Klra3 | 0.226113548 | 9.163693648 | 38.03066405 | 6.96415E−10 | 1.87325E−08 |
| Ipmk | −0.359137989 | 9.271383503 | 38.01288193 | 7.02791E−10 | 1.88726E−08 |
| Ebi3 | −0.24348842 | 9.178706953 | 37.89078919 | 7.48176E−10 | 2.00581E−08 |
| Lig1 | −0.326558724 | 9.221381095 | 37.80398986 | 7.82216E−10 | 2.09359E−08 |
| Ndc80 | −0.199325077 | 9.175316165 | 37.55760968 | 8.87525E−10 | 2.36761E−08 |
| Cphx3 | 0.057112739 | 9.157760229 | 37.56039337 | 8.86259E−10 | 2.36761E−08 |
| Fam64a | −0.106025888 | 9.164136557 | 37.51593717 | 9.06691E−10 | 2.41476E−08 |
| Bcl2l1 | −0.336680952 | 9.376460922 | 37.44850717 | 9.38585E−10 | 2.49149E−08 |
| Stat4 | −0.279257694 | 9.506546424 | 37.45009794 | 9.3782E−10 | 2.49149E−08 |

TABLE 3D
LPL−3 vs. All

| GeneSymbol | logFC | logCPM | LR | PValue | FDR |
|---|---|---|---|---|---|
| Ccl5 | −3.484093569 | 13.31083469 | 2049.126973 | 0 | 0 |
| Nkg7 | −2.068053995 | 11.21003636 | 2880.823851 | 0 | 0 |
| Plac8 | −2.112200427 | 10.56917796 | 1107.936846 | 6.221E−243 | 3.3523E−239 |
| Tmem176a | 1.889821585 | 9.404480898 | 1048.337873 | 5.5924E−230 | 2.2602E−226 |
| Ifitm2 | −2.051929407 | 10.68450167 | 928.579589 | 6.0127E−204 | 1.944E−200 |
| Tmem176b | 1.68947291 | 9.448057145 | 897.7209976 | 3.0708E−197 | 8.2737E−194 |
| Gzmb | −2.848731127 | 10.35041419 | 861.179545 | 2.6985E−189 | 6.2321E−186 |
| Gzma | −3.564634629 | 10.72609336 | 855.0832065 | 5.7078E−188 | 1.1534E−184 |
| Lgals3 | −1.660989425 | 10.20024122 | 735.8113412 | 4.8816E−162 | 8.7685E−159 |
| Ctsw | −1.058583981 | 9.943492884 | 639.5495471 | 4.187E−141 | 6.7688E−138 |
| Trib2 | 1.252838144 | 9.363791422 | 630.2041805 | 4.5125E−139 | 6.6317E−136 |
| Fgl2 | −1.622816533 | 9.496139008 | 629.1073924 | 7.8155E−139 | 1.0529E−135 |
| Ccr5 | −1.619827421 | 9.514363816 | 614.231888 | 1.3437E−135 | 1.671E−132 |
| mt-Co3 | 0.389203125 | 12.09929332 | 564.6764741 | 8.0786E−125 | 9.3285E−122 |
| Rgs1 | −1.059866606 | 11.08976227 | 547.5976498 | 4.1937E−121 | 4.5197E−118 |
| Rpl24 | 0.322646952 | 11.77231383 | 525.051429 | 3.3694E−116 | 3.4043E−113 |
| Ifitm3 | −1.878179626 | 9.717619666 | 520.6260984 | 3.0926E−115 | 2.9409E−112 |
| Rps27a | 0.315480197 | 11.80985986 | 518.9505848 | 7.1591E−115 | 6.4296E−112 |
| Rpl35 | 0.438027821 | 11.18862718 | 511.4426173 | 3.0785E−113 | 2.6193E−110 |
| mt-Atp6 | 0.346407175 | 12.47118005 | 484.3733011 | 2.3885E−107 | 1.9307E−104 |
| Il17re | 1.006347022 | 9.176312021 | 478.786329 | 3.925E−106 | 3.0215E−103 |
| Emb | 0.723801071 | 10.25108396 | 461.5739746 | 2.1846E−102 | 1.6053E−99 |
| Rps3a1 | 0.298969367 | 11.67402431 | 449.1348284 | 1.1128E−99 | 7.82125E−97 |
| AW112010 | −0.654555404 | 11.38311024 | 444.3256362 | 1.23889E−98 | 8.34493E−96 |
| mt-Co1 | 0.333063388 | 12.21547389 | 441.1231167 | 6.16614E−98 | 3.98727E−95 |
| Rps7 | 0.296343917 | 11.94322764 | 440.2941583 | 9.3417E−98 | 5.80838E−95 |
| Tcf7 | 0.970709967 | 9.419632902 | 430.9726984 | 9.98116E−96 | 5.97613E−93 |
| Serpinb1a | 1.594483756 | 9.512634337 | 428.3674149 | 3.68317E−95 | 2.1265E−92 |
| Rplp0 | 0.280821674 | 12.38790648 | 420.5795835 | 1.82513E−93 | 1.01741E−90 |
| Rps19 | 0.311069961 | 12.57927057 | 409.2190817 | 5.42142E−91 | 2.92142E−88 |
| Rpl31 | 0.392600539 | 10.8717304 | 403.7205395 | 8.53158E−90 | 4.44908E−87 |
| Fasl | −1.073589983 | 9.594179124 | 401.7278324 | 2.31638E−89 | 1.1702E−86 |
| Ifng | −1.773900792 | 9.68657816 | 393.9867729 | 1.12192E−87 | 5.49606E−85 |
| mt-Nd1 | 0.433097056 | 11.1492421 | 392.3866971 | 2.50204E−87 | 1.18965E−84 |
| Tnfrsf25 | 1.035091499 | 9.258890182 | 381.807873 | 5.02761E−85 | 2.32218E−82 |
| Rps29 | 0.309768839 | 12.24471351 | 380.2129007 | 1.11843E−84 | 5.02238E−82 |
| Satb1 | 0.803900794 | 9.757124872 | 378.0954162 | 3.23312E−84 | 1.41261E−81 |
| Slamf6 | 1.078369968 | 9.319064798 | 371.7261222 | 7.8771E−83 | 3.35108E−80 |
| Rpl4 | 0.268392668 | 11.68907178 | 368.4591631 | 4.05214E−82 | 1.67966E−79 |
| 1110034G24Rik | 1.031636667 | 9.229074688 | 368.1006541 | 4.85002E−82 | 1.96014E−79 |
| Rpl10 | 0.330381836 | 11.48679578 | 365.7282838 | 1.59329E−81 | 6.28221E−79 |
| Klrk1 | −1.466071219 | 9.564416625 | 363.1277724 | 5.86852E−81 | 2.25882E−78 |
| Rps8 | 0.298612024 | 11.59275565 | 354.4180219 | 4.62455E−79 | 1.73862E−76 |
| Rps18 | 0.253195601 | 12.47336872 | 353.1207929 | 8.8624E−79 | 3.25613E−76 |
| Rpl11 | 0.254557202 | 11.70912388 | 351.9326937 | 1.60795E−78 | 5.77646E−76 |
| Ramp3 | 1.163483877 | 9.937171094 | 350.6122351 | 3.11758E−78 | 1.09562E−75 |
| Litaf | −1.200479411 | 9.445010559 | 345.721109 | 3.62197E−77 | 1.2458E−74 |
| Rpl6 | 0.253049825 | 11.76090091 | 341.3669788 | 3.21491E−76 | 1.08275E−73 |
| Rpl32 | 0.241752504 | 12.44743739 | 338.9149259 | 1.09947E−75 | 3.56635E−73 |
| Hspa8 | −0.492721587 | 12.33067832 | 338.9084539 | 1.10304E−75 | 3.56635E−73 |
| Rpl18 | 0.257294066 | 11.80695531 | 335.2274387 | 6.98668E−75 | 2.21464E−72 |
| Rpl17 | 0.235452932 | 12.29667899 | 334.8509986 | 8.43833E−75 | 2.62335E−72 |
| Rps11 | 0.243194098 | 12.26312142 | 333.0602877 | 2.07139E−74 | 6.31813E−72 |
| Ccl4 | −2.829589103 | 9.567161703 | 332.3003568 | 3.0323E−74 | 9.0778E−72 |
| Dnajb1 | −1.447456963 | 10.31718294 | 330.6483343 | 6.94352E−74 | 2.04089E−71 |
| Nebl | 0.947797864 | 9.313896931 | 326.9696736 | 4.39345E−73 | 1.26829E−70 |
| mt-Co2 | 0.314373224 | 11.38674425 | 320.6458124 | 1.04768E−71 | 2.97138E−69 |
| Gzmk | −1.551161338 | 9.363616364 | 319.3540035 | 2.00269E−71 | 5.58198E−69 |
| Ctla2a | −0.96000106 | 10.33574783 | 318.1231264 | 3.71303E−71 | 1.01737E−68 |
| mt-Nd4 | 0.424844629 | 10.50325528 | 313.2764014 | 4.22177E−70 | 1.13749E−67 |
| AA467197 | −1.116233892 | 9.471396917 | 303.1631618 | 6.73964E−68 | 1.78612E−65 |
| Ccr6 | 0.698205543 | 9.169603333 | 297.721111 | 1.03341E−66 | 2.69452E−64 |
| mt-Cytb | 0.368527173 | 11.07880159 | 295.4669217 | 3.20185E−66 | 8.21605E−64 |
| Tox2 | 0.915454575 | 9.191330653 | 295.0827827 | 3.88237E−66 | 9.80661E−64 |
| Sh3bgrl3 | −0.377804436 | 11.24487265 | 294.6544492 | 4.81306E−66 | 1.19705E−63 |
| Rpl13a | 0.205282595 | 13.13152313 | 285.2357853 | 5.42831E−64 | 1.32961E−61 |
| Klrd1 | −1.254419995 | 9.372630202 | 281.7142906 | 3.17707E−63 | 7.66574E−61 |
| Naca | 0.316236433 | 10.80148809 | 280.0481025 | 7.33017E−63 | 1.74264E−60 |
| Rps14 | 0.200329253 | 12.65498588 | 275.9777944 | 5.65102E−62 | 1.32398E−59 |
| Cdkn1a | −1.052199877 | 9.693085153 | 274.133611 | 1.42571E−61 | 3.29258E−59 |
| Rpl13 | 0.20968469 | 12.63773823 | 272.0638942 | 4.02809E−61 | 9.17155E−59 |
| Rpl21 | 0.243080459 | 11.76877603 | 271.3049896 | 5.89515E−61 | 1.32363E−58 |
| Epsti1 | −0.650432645 | 9.945996659 | 268.5614751 | 2.33578E−60 | 5.17264E−58 |
| Rpl8 | 0.242856061 | 11.65231323 | 268.265358 | 2.71001E−60 | 5.92028E−58 |
| Rps9 | 0.221652232 | 11.99541867 | 264.7442415 | 1.58643E−59 | 3.41949E−57 |
| Nav2 | 0.696878506 | 9.1720222 | 262.7499097 | 4.31632E−59 | 9.18126E−57 |
| Rpl26 | 0.240002472 | 11.57434921 | 260.0823989 | 1.64648E−58 | 3.45674E−56 |
| Ms4a4b | −0.505784178 | 10.7517777 | 255.3500466 | 1.77064E−57 | 3.66976E−55 |
| Rpl35a | 0.231823918 | 11.72352561 | 254.4117161 | 2.83582E−57 | 5.80302E−55 |
| KIf2 | 1.050914377 | 9.96058395 | 253.5496162 | 4.37132E−57 | 8.83335E−55 |
| Dnaja1 | −0.649029266 | 10.76357472 | 252.0606719 | 9.23E−57 | 1.84213E−54 |
| Dapk2 | −0.98929305 | 9.267215042 | 251.000474 | 1.57155E−56 | 3.09826E−54 |
| Rps6 | 0.203360892 | 12.20845582 | 250.6981996 | 1.82905E−56 | 3.56247E−54 |
| Ccnd2 | −0.72559072 | 9.824522402 | 248.9497462 | 4.39953E−56 | 8.467E−54 |
| Rpl15 | 0.315788404 | 10.73598071 | 247.5305633 | 8.97036E−56 | 1.70606E−53 |
| Rps28 | 0.291807857 | 11.70754918 | 244.5326764 | 4.04034E−55 | 7.59489E−53 |
| Ccr2 | −0.829690745 | 9.897671261 | 243.2619236 | 7.64681E−55 | 1.4209E−52 |
| Il12rb2 | −0.988980941 | 9.345961023 | 240.1135399 | 3.71491E−54 | 6.82447E−52 |
| Tnfsf8 | 0.919482593 | 10.01800267 | 236.6741879 | 2.08882E−53 | 3.79415E−51 |
| 1700003C15Rik | 0.760033251 | 9.171905958 | 236.1461485 | 2.72298E−53 | 4.89108E−51 |
| Rpl34 | 0.203480775 | 11.98010645 | 235.5343091 | 3.70223E−53 | 6.57694E−51 |
| Il1r1 | 0.593316052 | 9.16767934 | 234.6804095 | 5.68417E−53 | 9.98807E−51 |
| Zfp3611 | 0.736388217 | 9.5364341 | 234.5048916 | 6.20785E−53 | 1.0791E−50 |
| Amica1 | −1.075213054 | 9.335190274 | 234.4351252 | 6.42917E−53 | 1.10568E−50 |
| Id2 | −0.621714646 | 10.64556657 | 232.0313476 | 2.14955E−52 | 3.65786E−50 |
| Serpina3g | −0.944522464 | 9.674077113 | 230.2129487 | 5.35676E−52 | 9.02056E−50 |
| Pim1 | −0.589343531 | 10.82520099 | 229.9043517 | 6.25463E−52 | 1.0424E−49 |
| Rps4x | 0.212878973 | 11.84808117 | 229.3253412 | 8.36518E−52 | 1.37991E−49 |
| Rpl10a | 0.271453577 | 11.14239123 | 227.5823075 | 2.0073E−51 | 3.27778E−49 |
| Rplp1 | 0.244800173 | 11.70340699 | 226.3395342 | 3.74676E−51 | 6.05702E−49 |
| Rps25 | 0.258258722 | 11.11877073 | 223.8649551 | 1.29829E−50 | 2.07803E−48 |
| Rps20 | 0.300464014 | 11.06882543 | 221.3182323 | 4.66495E−50 | 7.39349E−48 |
| H2-D1 | −0.231313769 | 12.28194321 | 220.6330101 | 6.58126E−50 | 1.03294E−47 |
| Sgip1 | 0.972538832 | 9.193478982 | 220.2322415 | 8.0487E−50 | 1.25111E−47 |
| Bhlhe40 | −0.446071264 | 10.72333649 | 218.3308818 | 2.09154E−49 | 3.22018E−47 |
| Hspa1a | −1.839599971 | 11.00088949 | 218.2514974 | 2.17662E−49 | 3.31956E−47 |
| Lysmd2 | 0.793775935 | 9.24333495 | 213.2812689 | 2.64248E−48 | 3.99237E−46 |
| Rpl18a | 0.178127329 | 12.84271416 | 213.0268771 | 3.00268E−48 | 4.49457E−46 |
| 1700012B07Rik | 0.635632788 | 9.170176807 | 211.5320497 | 6.36241E−48 | 9.43621E−46 |
| Znrf1 | 0.677706008 | 9.400584211 | 210.8496366 | 8.96398E−48 | 1.31738E−45 |
| Rps5 | 0.196579458 | 12.08892933 | 208.586472 | 2.79423E−47 | 4.06951E−45 |
| Rpl14 | 0.206279565 | 11.87709072 | 208.0441994 | 3.66923E−47 | 5.29615E−45 |
| Rps23 | 0.183689361 | 12.48535349 | 207.3464825 | 5.20961E−47 | 7.45297E−45 |
| Serpinb9 | −0.869185379 | 9.8583095 | 205.8671083 | 1.09544E−46 | 1.55341E−44 |
| Rps27 | 0.222000489 | 13.23482494 | 204.3505846 | 2.34686E−46 | 3.29907E−44 |
| Cmtm7 | −0.738958546 | 9.513298908 | 203.6105545 | 3.40379E−46 | 4.74359E−44 |
| Pde5a | 0.594157393 | 9.168289214 | 203.3978824 | 3.78764E−46 | 5.23341E−44 |
| Ubb | −0.320988013 | 12.90465858 | 202.2363742 | 6.78919E−46 | 9.3012E−44 |
| Smco4 | 0.707891435 | 9.324733933 | 202.1246558 | 7.18119E−46 | 9.75556E−44 |
| Hilpda | −0.79271988 | 9.667584429 | 195.5064907 | 1.99743E−44 | 2.69087E−42 |
| Gadd45b | −0.824371717 | 9.727231622 | 195.3857517 | 2.12237E−44 | 2.83556E−42 |
| Rcbtb2 | −0.897551208 | 9.398945286 | 194.1830275 | 3.88434E−44 | 5.14707E−42 |
| Slamf7 | −0.863101363 | 9.281089293 | 193.3260324 | 5.97533E−44 | 7.85344E−42 |
| Anxa2 | −0.662567838 | 9.842688336 | 192.9337152 | 7.27762E−44 | 9.48791E−42 |
| Rpl19 | 0.175722974 | 12.1175247 | 192.3267078 | 9.87363E−44 | 1.27694E−41 |
| Rpl22l1 | 0.315937439 | 10.87576896 | 192.041405 | 1.13959E−43 | 1.46211E−41 |
| Tuba1b | −0.817728304 | 9.730396626 | 189.0237562 | 5.1931E−43 | 6.61037E−41 |
| Ifitm1 | −2.386356518 | 9.599763038 | 188.2845877 | 7.52969E−43 | 9.50977E−41 |
| Icos | 0.472569335 | 10.25650654 | 188.1996714 | 7.85803E−43 | 9.84751E−41 |
| Rora | 0.507992709 | 9.982330628 | 187.9097328 | 9.09084E−43 | 1.13048E−40 |
| Rps24 | 0.205699287 | 12.0059475 | 184.8181341 | 4.30032E−42 | 5.30679E−40 |
| Smim3 | −0.828729834 | 9.350028927 | 184.7835494 | 4.37574E−42 | 5.35895E−40 |
| Klrc1 | −0.911521288 | 9.658426162 | 183.5897616 | 7.97391E−42 | 9.6922E−40 |
| Il1r2 | 1.118729196 | 9.256730933 | 183.3064645 | 9.19434E−42 | 1.10922E−39 |
| Xcl1 | −1.732920533 | 9.405068169 | 182.9886277 | 1.07872E−41 | 1.29175E−39 |
| Il22 | 1.870572455 | 9.482887799 | 182.8520365 | 1.1554E−41 | 1.37339E−39 |
| Plek | −0.751460525 | 9.215817596 | 182.2332023 | 1.57702E−41 | 1.86089E−39 |
| Rps2 | 0.235163135 | 11.63594659 | 180.5370278 | 3.6997E−41 | 4.33401E−39 |
| Fosb | −1.0044227 | 9.410738732 | 179.1890367 | 7.28599E−41 | 8.47377E−39 |
| Lilr4b | −0.647414806 | 9.778128629 | 177.1360399 | 2.04534E−40 | 2.36179E−38 |
| Rpl12 | 0.278475812 | 10.9924871 | 176.0772239 | 3.48315E−40 | 3.99352E−38 |
| Rassf3 | 0.683581715 | 9.199068087 | 176.0013684 | 3.61857E−40 | 4.11956E−38 |
| Rps16 | 0.174627684 | 12.35142977 | 175.5750544 | 4.48364E−40 | 5.06871E−38 |
| Cldnd1 | −0.684362495 | 9.795115659 | 175.4406393 | 4.79715E−40 | 5.38546E−38 |
| Ctsd | −0.594814309 | 9.64172066 | 172.9552202 | 1.67397E−39 | 1.86631E−37 |
| Rpl23 | 0.203421469 | 11.70878799 | 170.7965908 | 4.95662E−39 | 5.48827E−37 |
| Rgs10 | 0.539364334 | 9.631678923 | 169.9400775 | 7.62525E−39 | 8.3857E−37 |
| S1pr1 | 0.756906257 | 9.290346876 | 168.9861794 | 1.23197E−38 | 1.34567E−36 |
| Dusp5 | −0.50010877 | 9.994364417 | 168.5445148 | 1.53839E−38 | 1.6691E−36 |
| Sptssa | 0.530775409 | 9.597728351 | 168.3290064 | 1.7145E−38 | 1.84778E−36 |
| Lsp1 | 0.371113465 | 10.32147684 | 167.8015721 | 2.23534E−38 | 2.39314E−36 |
| Tmsb4x | −0.190326855 | 14.24925577 | 166.3632964 | 4.60795E−38 | 4.9008E−36 |
| Icam1 | −0.836752343 | 9.404341293 | 166.161961 | 5.09903E−38 | 5.38764E−36 |
| Rps26 | 0.255375413 | 10.99418168 | 166.0950448 | 5.27357E−38 | 5.53588E−36 |
| Socs2 | −0.893608998 | 9.351698274 | 165.9933737 | 5.55026E−38 | 5.78874E−36 |
| Rpa2 | −0.768064662 | 9.392655105 | 164.4327646 | 1.21681E−37 | 1.26096E−35 |
| Rps3 | 0.163431739 | 11.96715944 | 163.6126366 | 1.83816E−37 | 1.89272E−35 |
| Rpl41 | 0.148484636 | 12.79527095 | 163.2689082 | 2.18512E−37 | 2.23574E−35 |
| Tspan13 | 0.526355965 | 9.546671007 | 162.7547983 | 2.83002E−37 | 2.87736E−35 |
| Cd69 | −0.700203337 | 9.886218478 | 161.6594313 | 4.91014E−37 | 4.96108E−35 |
| Odc1 | 0.530928101 | 10.453789 | 160.6935792 | 7.982E−37 | 8.01472E−35 |
| Bcl2l1 | −0.778019491 | 9.376464206 | 159.4570551 | 1.48688E−36 | 1.48376E−34 |
| Mrps5 | 0.67574663 | 9.263071627 | 158.1039542 | 2.93715E−36 | 2.91301E−34 |
| Hey1 | 0.339150364 | 9.161115987 | 157.942378 | 3.1859E−36 | 3.14044E−34 |
| Glrx | −0.646284655 | 9.565877632 | 157.1553176 | 4.73381E−36 | 4.63799E−34 |
| Abi3 | −0.803877473 | 9.334617896 | 156.9668392 | 5.2047E−36 | 5.06863E−34 |
| H2afy | −0.435862619 | 9.9300789 | 156.0855564 | 8.10908E−36 | 7.84978E−34 |
| Samsn1 | −0.607961392 | 9.795508071 | 156.0048309 | 8.44524E−36 | 8.12653E−34 |
| Rps15 | 0.19771985 | 11.49082426 | 154.2349025 | 2.05775E−35 | 1.96838E−33 |
| Rpl29 | 0.219501544 | 11.21441306 | 152.8746442 | 4.08009E−35 | 3.87992E−33 |
| Gmfg | 0.332354389 | 10.21757862 | 152.1259771 | 5.94693E−35 | 5.62211E−33 |
| Limd2 | 0.389855996 | 10.089006 | 150.8366734 | 1.13785E−34 | 1.06945E−32 |
| Rpl23a | 0.168220353 | 12.17564707 | 150.4212869 | 1.40242E−34 | 1.31049E−32 |
| Rpl28 | 0.263182468 | 10.72582102 | 149.6866768 | 2.02976E−34 | 1.88581E−32 |
| Lilrb4a | −0.653757891 | 9.646924972 | 148.6586133 | 3.40536E−34 | 3.14578E−32 |
| Ftl1 | 0.286104977 | 10.82038569 | 148.5083679 | 3.67287E−34 | 3.37361E−32 |
| Cd3g | −0.263497128 | 11.10927275 | 148.2266944 | 4.23231E−34 | 3.86551E−32 |
| Arhgap31 | 0.539802079 | 9.654196025 | 146.6272891 | 9.46691E−34 | 8.59787E−32 |
| Serpinb6b | −0.694956176 | 9.622268614 | 145.5103981 | 1.66103E−33 | 1.50012E−31 |
| Tbx21 | −0.741006971 | 9.277656746 | 144.9687718 | 2.18167E−33 | 1.95938E−31 |
| Tmem64 | 0.7402499 | 9.248102293 | 143.79394 | 3.94133E−33 | 3.5202E−31 |
| Gnb2l1 | 0.257536167 | 10.54032337 | 143.4136363 | 4.77301E−33 | 4.23959E−31 |
| Tm6sf1 | −0.614151136 | 9.205991409 | 141.0728903 | 1.55097E−32 | 1.37011E−30 |
| Gpr171 | −0.527919467 | 9.722719309 | 140.898755 | 1.69309E−32 | 1.48753E−30 |
| Ass1 | 0.557775131 | 9.534395587 | 140.432394 | 2.14121E−32 | 1.87107E−30 |
| Hsp90aa1 | −0.551436005 | 10.7686095 | 140.4135022 | 2.16168E−32 | 1.8788E−30 |
| Nsg2 | 0.457632765 | 9.167222657 | 139.9230257 | 2.76724E−32 | 2.39225E−30 |
| Rpl22 | 0.24172144 | 10.60720364 | 139.3472402 | 3.69794E−32 | 3.17983E−30 |
| Krtcap2 | −0.356333163 | 10.06414216 | 139.1868613 | 4.00896E−32 | 3.42904E−30 |
| Stk24 | 0.468770685 | 9.608428218 | 138.9674266 | 4.47733E−32 | 3.79489E−30 |
| Rgcc | −0.900503044 | 9.563503156 | 138.9646335 | 4.48364E−32 | 3.79489E−30 |
| Gpr34 | −0.757646242 | 9.25651743 | 138.0295522 | 7.18003E−32 | 6.04544E−30 |
| St6galnac1 | 0.564245409 | 9.173614926 | 137.6712982 | 8.59957E−32 | 7.20314E−30 |
| Pkp4 | 0.568152144 | 9.180314385 | 136.6038442 | 1.4721E−31 | 1.2267E−29 |
| Slpi | 0.486549059 | 9.166637373 | 135.5644851 | 2.48466E−31 | 2.05985E−29 |
| Cxcr3 | 0.531979192 | 9.551340066 | 134.7179405 | 3.80568E−31 | 3.13891E−29 |
| Lmna | −0.825151176 | 9.713615726 | 133.8629815 | 5.85392E−31 | 4.80378E−29 |
| Rpl7 | 0.189257523 | 11.21210542 | 133.217726 | 8.10208E−31 | 6.61506E−29 |
| 2810417H13Rik | −1.285204722 | 9.337765738 | 132.6784218 | 1.0631E−30 | 8.63619E−29 |
| S100a4 | 0.670964086 | 9.866831921 | 132.345813 | 1.257E−30 | 1.01603E−28 |
| Ifi27l2a | 0.667189776 | 9.81722567 | 132.2788965 | 1.30009E−30 | 1.04564E−28 |
| Tubb5 | −0.571247104 | 10.00333821 | 131.589452 | 1.83994E−30 | 1.4725E−28 |
| Birc5 | −0.886968249 | 9.251705243 | 131.3715948 | 2.05336E−30 | 1.6352E−28 |
| Txn1 | −0.38477823 | 10.00228125 | 130.9094939 | 2.59157E−30 | 2.04367E−28 |
| Dtx1 | 0.710488782 | 9.250196625 | 130.9121903 | 2.58805E−30 | 2.04367E−28 |
| Mast4 | 0.551969332 | 9.181692037 | 129.6748765 | 4.82707E−30 | 3.78808E−28 |
| Cd38 | −0.722178037 | 9.272404605 | 129.2849214 | 5.87497E−30 | 4.58815E−28 |
| Iltifb | 0.466521316 | 9.164994613 | 128.6602113 | 8.04814E−30 | 6.25511E−28 |
| Plcb4 | 0.396482666 | 9.163609867 | 128.5931213 | 8.32484E−30 | 6.4392E−28 |
| Eef1a1 | 0.151323644 | 12.38193961 | 127.3339193 | 1.57005E−29 | 1.20864E−27 |
| Gbp2 | −0.762105085 | 9.365780295 | 126.7822278 | 2.07319E−29 | 1.5884E−27 |
| Rpl3 | 0.150394133 | 12.09257424 | 126.6318569 | 2.23638E−29 | 1.70535E−27 |
| Ccr7 | 1.097629633 | 9.273175644 | 125.9374899 | 3.17323E−29 | 2.40838E−27 |
| Nr4a2 | −0.69869436 | 9.521215332 | 125.4110146 | 4.13733E−29 | 3.12542E−27 |
| Rplp2 | 0.17351654 | 11.91020443 | 124.8852816 | 5.39237E−29 | 4.05456E−27 |
| Rps10 | 0.188460525 | 11.22201226 | 123.7513581 | 9.549E−29 | 7.14672E−27 |
| Eif3f | 0.224777999 | 10.6645601 | 123.6885264 | 9.85622E−29 | 7.34265E−27 |
| Klrg1 | −0.619319207 | 9.202815326 | 123.5187912 | 1.07365E−28 | 7.96173E−27 |
| Ppia | −0.201559198 | 11.56548656 | 122.9632193 | 1.42058E−28 | 1.04863E−26 |
| mt-Nd2 | 0.348333644 | 9.930964526 | 122.1357147 | 2.15576E−28 | 1.58409E−26 |
| Cxcr6 | −0.460498452 | 10.38993977 | 122.1090465 | 2.18493E−28 | 1.59826E−26 |
| Stmn1 | −1.215251954 | 9.430719181 | 120.6930632 | 4.46074E−28 | 3.2483E−26 |
| Hspa1b | −1.061056122 | 9.330476915 | 120.3110025 | 5.40813E−28 | 3.92053E−26 |
| Plscr1 | −0.642118473 | 9.521580006 | 119.2788005 | 9.09973E−28 | 6.56724E−26 |
| Ccnd3 | −0.536704025 | 9.527398394 | 118.8178614 | 1.14801E−27 | 8.24835E−26 |
| Izumo1r | 0.73550348 | 9.250980666 | 118.2124259 | 1.55778E−27 | 1.1143E−25 |
| Cdk5r1 | 0.576455527 | 9.178698472 | 117.6513688 | 2.06707E−27 | 1.47208E−25 |
| Eef1b2 | 0.295763405 | 10.0755718 | 117.2418803 | 2.54109E−27 | 1.80172E−25 |
| Camk2n1 | −0.685364499 | 9.340459445 | 117.1854268 | 2.61445E−27 | 1.84564E−25 |
| Nfkbia | −0.443575026 | 10.83336515 | 117.0602753 | 2.78475E−27 | 1.95731E−25 |
| 2010111l01Rik | −0.58976034 | 9.402743483 | 116.5749773 | 3.55676E−27 | 2.48911E−25 |
| Rps17 | 0.255513344 | 10.61526354 | 115.2722497 | 6.86021E−27 | 4.77841E−25 |
| Mxd1 | −0.561117916 | 9.600457879 | 115.2644894 | 6.88711E−27 | 4.77841E−25 |
| Gngt2 | −0.645521342 | 9.363421412 | 114.291152 | 1.12514E−26 | 7.73998E−25 |
| Eif3e | 0.276298263 | 10.13949795 | 114.293671 | 1.12371E−26 | 7.73998E−25 |
| Rpl9 | 0.148253371 | 11.84229891 | 113.9941505 | 1.30693E−26 | 8.9525E−25 |
| Ccr4 | 0.593390397 | 9.227425335 | 113.5628703 | 1.62448E−26 | 1.10807E−24 |
| Txk | 0.567145719 | 9.335340173 | 113.2795033 | 1.87405E−26 | 1.27293E−24 |
| Aqp3 | 0.501331766 | 9.177291322 | 112.4084827 | 2.90782E−26 | 1.96685E−24 |
| S100a11 | −0.300484266 | 10.60261953 | 112.3500062 | 2.99486E−26 | 2.01729E−24 |
| Pecam1 | 0.57408471 | 9.257221083 | 111.8795665 | 3.79688E−26 | 2.5469E−24 |
| Avpi1 | 0.587850586 | 9.200072787 | 111.4995665 | 4.59906E−26 | 3.07225E−24 |
| Ly6c2 | −1.077631275 | 9.266017563 | 111.3836024 | 4.8761E−26 | 3.24391E−24 |
| Rpl36a | 0.210891627 | 11.04534811 | 109.1644599 | 1.49364E−25 | 9.896E−24 |
| Tnfrsf26 | 0.569766024 | 9.206961652 | 107.6619109 | 3.1877E−25 | 2.10336E−23 |
| Rbpj | −0.56851782 | 9.385158504 | 107.1608345 | 4.10469E−25 | 2.69741E−23 |
| Smpdl3a | 0.418240822 | 9.556683298 | 106.5726921 | 5.5229E−25 | 3.61471E−23 |
| Fau | 0.133036285 | 12.30167072 | 106.2430132 | 6.52254E−25 | 4.25175E−23 |
| Slbp | 0.483341551 | 9.609796597 | 106.0970615 | 7.02107E−25 | 4.55834E−23 |
| Efhd2 | −0.507188907 | 9.465456867 | 105.6326427 | 8.87536E−25 | 5.73916E−23 |
| Actn1 | 0.575522456 | 9.187194301 | 105.6017048 | 9.01501E−25 | 5.80624E−23 |
| Itga1 | −0.481159298 | 9.197482484 | 104.95344 | 1.2504E−24 | 8.0214E−23 |
| Junb | −0.334624697 | 11.89080177 | 104.8572636 | 1.31259E−24 | 8.38706E−23 |
| Tspo | 0.314378756 | 9.918101619 | 104.6270095 | 1.47433E−24 | 9.38349E−23 |
| Dusp10 | 0.58029624 | 9.475539651 | 104.0297313 | 1.99303E−24 | 1.2635E−22 |
| Rpl30 | 0.19389992 | 10.96422059 | 103.0676934 | 3.2389E−24 | 2.04531E−22 |
| Lztfl1 | −0.623767739 | 9.420902283 | 102.5746732 | 4.1541E−24 | 2.61304E−22 |
| Dennd4a | −0.578405941 | 9.353169382 | 102.5290984 | 4.25078E−24 | 2.66349E−22 |
| 5430421N21Rik | 0.646852187 | 9.214329084 | 102.1476476 | 5.1534E−24 | 3.2166E−22 |
| Ldha | 0.277341313 | 10.35628782 | 101.9060758 | 5.82177E−24 | 3.6198E−22 |
| Eomes | −0.779813416 | 9.215476434 | 101.3944563 | 7.53744E−24 | 4.66859E−22 |
| Ppp1cc | 0.303541918 | 10.14034874 | 99.89229099 | 1.60914E−23 | 9.92879E−22 |
| Malat1 | −0.254811632 | 14.66845899 | 99.87543084 | 1.6229E−23 | 9.97559E−22 |
| Gpr18 | −0.656361909 | 9.406528173 | 99.35693704 | 2.10858E−23 | 1.29119E−21 |
| H2-K1 | 0.198523334 | 11.61002829 | 98.90320569 | 2.6515E−23 | 1.61751E−21 |
| Runx3 | −0.471586573 | 9.557610794 | 97.59444435 | 5.13478E−23 | 3.12063E−21 |
| Tmsb10 | 0.154827346 | 12.94248991 | 97.16784513 | 6.36926E−23 | 3.85639E−21 |
| Lag3 | −0.863184819 | 9.272147436 | 97.12994873 | 6.49234E−23 | 3.91624E−21 |
| Cpm | 0.566098049 | 9.24931383 | 97.09275961 | 6.61544E−23 | 3.97566E−21 |
| Ms4a6b | −0.333588841 | 10.1948354 | 96.00750204 | 1.14449E−22 | 6.85253E−21 |
| H2-Q7 | −0.264138572 | 10.62105384 | 95.84377435 | 1.24316E−22 | 7.41586E−21 |
| Rpl27a | 0.13805365 | 11.7398871 | 94.43658567 | 2.53071E−22 | 1.5041E−20 |
| Gas7 | −0.461230762 | 9.194247453 | 94.25268389 | 2.7771E−22 | 1.64449E−20 |
| Tecpr1 | 0.465177325 | 9.413068687 | 93.95828779 | 3.22244E−22 | 1.90124E−20 |
| Tsc22d1 | 0.514971286 | 9.170828337 | 93.63509947 | 3.794E−22 | 2.23032E−20 |
| Rorc | 0.340741054 | 9.165458039 | 92.99485602 | 5.24304E−22 | 3.07098E−20 |
| Ptrh1 | 0.416669722 | 9.177194315 | 92.97032326 | 5.30844E−22 | 3.09806E−20 |
| Dhrs3 | 0.469692706 | 9.174233573 | 92.76398048 | 5.89178E−22 | 3.42613E−20 |
| Nfkbiz | −0.472625752 | 9.633382235 | 92.64171855 | 6.26723E−22 | 3.6314E−20 |
| Gnas | −0.249759348 | 10.37740546 | 92.59947857 | 6.40243E−22 | 3.69649E−20 |
| Il2ra | −0.594584914 | 9.266141063 | 91.94031851 | 8.93303E−22 | 5.1392E−20 |
| Slc9a3r1 | −0.397597403 | 9.651360086 | 91.91314639 | 9.05654E−22 | 5.19177E−20 |
| Smoc2 | 0.217368642 | 9.159952973 | 91.81873843 | 9.49907E−22 | 5.42622E−20 |
| Asb2 | −0.566764352 | 9.480727364 | 91.47295697 | 1.13128E−21 | 6.43952E−20 |
| Ccnb2 | −0.574479423 | 9.212362432 | 91.21075062 | 1.29157E−21 | 7.32613E−20 |
| Chst12 | −0.591913388 | 9.307931014 | 91.00443213 | 1.43351E−21 | 8.10282E−20 |
| Dusp2 | −0.651560783 | 9.446121177 | 90.7507899 | 1.62956E−21 | 9.17892E−20 |
| Rexo2 | 0.41801584 | 9.465243005 | 89.60388759 | 2.90952E−21 | 1.63317E−19 |
| Hsph1 | −0.62803205 | 9.472358293 | 88.22977687 | 5.8276E−21 | 3.25983E−19 |
| Arf6 | −0.341525694 | 9.853237401 | 87.74031434 | 7.46375E−21 | 4.16066E−19 |
| Cd84 | 0.465838199 | 9.34855222 | 87.58985183 | 8.05368E−21 | 4.47408E−19 |
| Gbp3 | −0.575087315 | 9.305894756 | 86.67276617 | 1.28048E−20 | 7.08912E−19 |
| Cyb5a | 0.418826942 | 9.520944338 | 86.4705939 | 1.41831E−20 | 7.82537E−19 |
| Traf4 | −0.547865622 | 9.359798586 | 85.45465565 | 2.37076E−20 | 1.3036E−18 |
| Prkcb | 0.470193718 | 9.208819069 | 84.45661779 | 3.92738E−20 | 2.15221E−18 |
| Lrrc25 | 0.258088384 | 9.161329097 | 84.44163993 | 3.95725E−20 | 2.16125E−18 |
| Sh2d2a | −0.380576774 | 9.846351053 | 84.3959534 | 4.04976E−20 | 2.20432E−18 |
| Hmgn2 | −0.601700458 | 9.445400237 | 83.92876523 | 5.12928E−20 | 2.78255E−18 |
| Rhoc | −0.562347362 | 9.244274494 | 83.85510055 | 5.32401E−20 | 2.87852E−18 |
| Acsbg1 | 0.47503696 | 9.314382446 | 83.81601429 | 5.43031E−20 | 2.92622E−18 |
| Dkkl1 | −0.529480805 | 9.308686946 | 83.51509157 | 6.32314E−20 | 3.39601E−18 |
| Hcst | −0.252513111 | 10.61089653 | 83.25678198 | 7.20579E−20 | 3.85724E−18 |
| Acad | −0.497894127 | 9.426499963 | 83.21766254 | 7.34981E−20 | 3.92135E−18 |
| Asap1 | 0.515125763 | 9.22985191 | 82.67182493 | 9.68723E−20 | 5.15144E−18 |
| Cox7a21 | 0.245914858 | 10.13150038 | 82.17709101 | 1.24424E−19 | 6.59486E−18 |
| Ap3s1 | 0.392394745 | 9.478733316 | 82.12212471 | 1.27932E−19 | 6.75868E−18 |
| Shisa5 | 0.193645712 | 11.05064206 | 82.01459451 | 1.35085E−19 | 7.11333E−18 |
| Ltb | 0.362820009 | 10.15488605 | 81.87811624 | 1.44743E−19 | 7.59713E−18 |
| Ccna2 | −0.403785662 | 9.193840202 | 81.36484801 | 1.87666E−19 | 9.81817E−18 |
| Mns1 | −0.536056376 | 9.250789284 | 81.30652077 | 1.93288E−19 | 1.00796E−17 |
| Csf1 | −0.510822609 | 9.192100854 | 81.19288199 | 2.04728E−19 | 1.06419E−17 |
| Ramp1 | 0.361595708 | 9.166176693 | 80.89429173 | 2.38121E−19 | 1.2338E−17 |
| Cyba | −0.238796652 | 10.43095334 | 80.73209482 | 2.5849E−19 | 1.33506E−17 |
| Ptms | −0.527438745 | 9.287856152 | 80.5814284 | 2.78969E−19 | 1.43625E−17 |
| Ccl3 | −0.910093878 | 9.220257049 | 80.47423238 | 2.94521E−19 | 1.5115E−17 |
| Ppm1j | −0.509834666 | 9.231926591 | 80.0920149 | 3.57374E−19 | 1.82826E−17 |
| Asf1b | −0.539633023 | 9.223320297 | 80.00113282 | 3.74195E−19 | 1.90828E−17 |
| Rpl39 | 0.140509529 | 11.77739482 | 79.99106699 | 3.76106E−19 | 1.91199E−17 |
| Eif3h | 0.207162722 | 10.29703651 | 79.51031985 | 4.79713E−19 | 2.43105E−17 |
| Itm2b | −0.202508228 | 10.97659374 | 79.05171761 | 6.05051E−19 | 3.05664E−17 |
| Rpl5 | 0.195642628 | 10.56774531 | 78.40370728 | 8.39944E−19 | 4.23007E−17 |
| Mt1 | −0.681149163 | 9.25333207 | 78.01199452 | 1.02417E−18 | 5.14183E−17 |
| Slc2a3 | −0.535215118 | 9.267489223 | 77.88899828 | 1.08997E−18 | 5.45524E−17 |
| Jund | −0.291126203 | 11.83355423 | 77.37694158 | 1.41255E−18 | 7.04791E−17 |
| Vcam1 | 0.41406013 | 9.169856475 | 77.29191905 | 1.47468E−18 | 7.33529E−17 |
| Cxcl3 | 0.555799907 | 9.168367119 | 77.24424832 | 1.51071E−18 | 7.49144E−17 |
| Ralgps2 | 0.447656391 | 9.202191153 | 77.21516413 | 1.53312E−18 | 7.57933E−17 |
| Atf3 | −0.680296415 | 9.293174358 | 76.62548982 | 2.06655E−18 | 1.01853E−16 |
| Osgin1 | −0.593267168 | 9.241885952 | 76.07875288 | 2.72574E−18 | 1.33934E−16 |
| Ctla2b | −0.522490544 | 9.270070138 | 76.04275853 | 2.77588E−18 | 1.35985E−16 |
| Faah | 0.44588788 | 9.275977389 | 75.14662319 | 4.37024E−18 | 2.13442E−16 |
| Prr7 | −0.393644523 | 9.824437204 | 75.07309927 | 4.53605E−18 | 2.20873E−16 |
| Tnfrsf18 | −0.323439954 | 10.06784361 | 74.91937037 | 4.90336E−18 | 2.38041E−16 |
| Fam101b | 0.44395883 | 9.174664735 | 74.87849381 | 5.00593E−18 | 2.42293E−16 |
| Sla | 0.378547652 | 9.546204364 | 74.52405875 | 5.99038E−18 | 2.89076E−16 |
| Cdca3 | −0.416722054 | 9.196808089 | 73.81078112 | 8.59757E−18 | 4.13656E−16 |
| Lgals1 | −0.288816159 | 11.62800987 | 73.67765585 | 9.19741E−18 | 4.41203E−16 |
| Stk32c | −0.48699157 | 9.236288863 | 73.53762236 | 9.87362E−18 | 4.72239E−16 |
| Gm9493 | 0.236255264 | 10.33364392 | 73.33101042 | 1.09632E−17 | 5.22805E−16 |
| Rrm2 | −0.656260613 | 9.247707962 | 73.06368613 | 1.25533E−17 | 5.96873E−16 |
| Gm8730 | 0.223093492 | 10.44488374 | 72.87273433 | 1.38285E−17 | 6.55579E−16 |
| Serpina3f | −0.661617841 | 9.259699948 | 72.26884197 | 1.87789E−17 | 8.8766E−16 |
| Il14ra | −0.441442073 | 9.499543146 | 71.80778832 | 2.37214E−17 | 1.11802E−15 |
| Ckb | 0.483538637 | 9.335721648 | 71.61412486 | 2.61677E−17 | 1.22973E−15 |
| Acp5 | 0.397423986 | 9.396912844 | 71.56913307 | 2.67712E−17 | 1.25185E−15 |
| Rcn1 | −0.444001155 | 9.211899643 | 71.5675056 | 2.67933E−17 | 1.25185E−15 |
| Spc24 | −0.443249395 | 9.204195345 | 70.62308424 | 4.32427E−17 | 2.01459E−15 |
| Rps12 | 0.192586529 | 10.77092803 | 70.24630421 | 5.23433E−17 | 2.43155E−15 |
| Clta | −0.284608639 | 9.8733927 | 70.18241162 | 5.40664E−17 | 2.5044E−15 |
| Skil | −0.428945944 | 9.558639486 | 69.82061796 | 6.49504E−17 | 2.99997E−15 |
| Cdkn2c | −0.492927169 | 9.239141777 | 69.80701627 | 6.53998E−17 | 3.01212E−15 |
| 2310001H17Rik | 0.45313487 | 9.290442993 | 69.72339182 | 6.82321E−17 | 3.13364E−15 |
| Egln3 | 0.460826066 | 9.20786883 | 69.16184134 | 9.07061E−17 | 4.15398E−15 |
| Ncor1 | 0.309981143 | 9.664996809 | 69.00893228 | 9.80185E−17 | 4.47618E−15 |
| Tnfrsf9 | −0.607969904 | 9.402131727 | 68.99666734 | 9.863E−17 | 4.49141E−15 |
| Prelid2 | 0.424695126 | 9.192055381 | 68.68295117 | 1.15636E−16 | 5.25106E−15 |
| Padi2 | −0.447711328 | 9.215639868 | 67.88323124 | 1.7347E−16 | 7.85524E−15 |
| Ndufa4 | 0.305255781 | 9.735529358 | 67.70371654 | 1.90005E−16 | 8.57995E−15 |
| Gbp7 | −0.476561834 | 9.290910914 | 67.67068329 | 1.93215E−16 | 8.7006E−15 |
| Ube2c | −0.631545281 | 9.233527278 | 67.5201665 | 2.08543E−16 | 9.36474E−15 |
| Ifrd1 | −0.336527284 | 10.02583543 | 67.17457762 | 2.48498E−16 | 1.1128E−14 |
| Cdk1 | −0.39769185 | 9.19735213 | 67.15632591 | 2.50809E−16 | 1.12005E−14 |
| Tuba4a | −0.464387774 | 9.389389589 | 67.02800208 | 2.67678E−16 | 1.19209E−14 |
| Neurl3 | −0.393731218 | 9.619600065 | 66.75753682 | 3.07041E−16 | 1.36363E−14 |
| Cnn3 | 0.198623534 | 9.160462978 | 66.70096425 | 3.1598E−16 | 1.39949E−14 |
| Cd47 | −0.236561159 | 10.2358714 | 66.62087045 | 3.29083E−16 | 1.45354E−14 |
| Pelo | −0.459502434 | 9.234978423 | 66.57690441 | 3.36506E−16 | 1.48228E−14 |
| Ubc | −0.246876595 | 11.04983306 | 66.51122535 | 3.47907E−16 | 1.52833E−14 |
| Ptma | −0.194475287 | 11.28840747 | 66.30598276 | 3.86085E−16 | 1.69145E−14 |
| Itgb1 | −0.6429514 | 9.29928184 | 66.26452004 | 3.94292E−16 | 1.72274E−14 |
| Rpl37a | 0.114612104 | 12.61563941 | 66.16203022 | 4.15337E−16 | 1.80979E−14 |
| Ptger4 | −0.503230593 | 9.34461786 | 66.11246381 | 4.25914E−16 | 1.85089E−14 |
| Rps15a | 0.109891473 | 12.40383431 | 66.08931246 | 4.30946E−16 | 1.86774E−14 |
| Ccl1 | −1.01837332 | 9.258139682 | 65.45285896 | 5.95208E−16 | 2.57276E−14 |
| Tcp11l2 | 0.407869719 | 9.433884628 | 65.28344021 | 6.48641E−16 | 2.79625E−14 |
| Eif3k | 0.209698805 | 10.09230815 | 65.11697117 | 7.05816E−16 | 3.03463E−14 |

TABLE 3E
LPL−4 vs. All

| GeneSymbol | logFC | logCPM | LR | PValue | FDR |
|---|---|---|---|---|---|
| Jun | 2.590143686 | 9.603439503 | 1533.659276 | 0 | 0 |
| Klf6 | 1.545036169 | 9.89686973 | 1361.417237 | 5.089E−298 | 4.1134E−294 |
| Dusp1 | 1.611063957 | 9.939042764 | 1258.200347 | 1.3708E−275 | 7.387E−272 |
| Fos | 2.334554203 | 9.517129944 | 1248.343188 | 1.9017E−273 | 7.6858E−270 |
| Ubb | 0.716874178 | 12.90465449 | 1126.424498 | 5.9667E−247 | 1.9291E−243 |
| Rgs1 | 1.270826473 | 11.08975568 | 1112.879546 | 5.2433E−244 | 1.4127E−240 |
| Hspa1a | 2.826179586 | 11.00085583 | 1078.202577 | 1.805E−236 | 4.1685E−233 |
| Ubc | 0.914435003 | 11.0498222 | 1066.356992 | 6.778E−234 | 1.3697E−230 |
| Dnajb1 | 1.892963828 | 10.31714857 | 948.4998693 | 2.8108E−208 | 5.0489E−205 |
| Cd69 | 1.363050626 | 9.886202315 | 884.9866185 | 1.8013E−194 | 2.9119E−191 |
| Zfp36 | 1.848813098 | 9.294559476 | 835.0942821 | 1.2651E−183 | 1.8593E−180 |
| Hspa1b | 2.15174286 | 9.330468231 | 793.0459723 | 1.7539E−174 | 2.3628E−171 |
| Hspa8 | 0.696470563 | 12.33068176 | 751.3892978 | 2.0013E−165 | 2.4887E−162 |
| Nfkbia | 0.985907956 | 10.83334808 | 699.2477201 | 4.3581E−154 | 5.0323E−151 |
| Gadd45b | 1.286294082 | 9.727225725 | 674.2268837 | 1.2033E−148 | 1.2969E−145 |
| H2afz | −1.07018428 | 10.76614048 | 664.4674558 | 1.5951E−146 | 1.6116E−143 |
| Junb | 0.793003216 | 11.89077788 | 658.8329868 | 2.68E−145 | 2.5485E−142 |
| Ppp1r15a | 1.114516956 | 9.595202536 | 607.3150774 | 4.2927E−134 | 3.8553E−131 |
| Ifngr1 | −0.797175635 | 11.1130316 | 606.1095339 | 7.8512E−134 | 6.6802E−131 |
| Tnfaip3 | 0.803794065 | 10.12471168 | 549.5002817 | 1.617E−121 | 1.307E−118 |
| Tagap | 1.261551553 | 9.424665079 | 531.4945681 | 1.336E−117 | 1.0284E−114 |
| Phlda1 | 1.400498169 | 9.440744496 | 524.7965068 | 3.8283E−116 | 2.8131E−113 |
| Gramd3 | 1.16036844 | 10.20636507 | 497.1879766 | 3.8888E−110 | 2.7333E−107 |
| Hsp90aa1 | 0.935956122 | 10.76861769 | 471.3892672 | 1.5976E−104 | 1.0761E−101 |
| Fosb | 1.329877314 | 9.41073207 | 435.298814 | 1.14187E−96 | 7.38381E−94 |
| ler5 | 0.885845247 | 9.710298508 | 407.4325977 | 1.32736E−90 | 8.25311E−88 |
| Tsc22d3 | 0.984645148 | 9.704342129 | 404.9225279 | 4.67066E−90 | 2.79652E−87 |
| Hsp90ab1 | 0.633814222 | 11.19704142 | 400.9346148 | 3.44732E−89 | 1.99033E−86 |
| Emb | −0.904301807 | 10.2510913 | 391.702411 | 3.52584E−87 | 1.96547E−84 |
| Serpinb9 | −1.366273321 | 9.858316986 | 378.3861841 | 2.79458E−84 | 1.50591E−81 |
| Asb2 | 1.034237011 | 9.480724756 | 375.4102947 | 1.2423E−83 | 6.47842E−81 |
| Ramp3 | −1.695838734 | 9.93719207 | 371.2181081 | 1.01619E−82 | 5.13367E−80 |
| Bag3 | 1.280714683 | 9.20292133 | 370.6579391 | 1.34568E−82 | 6.59218E−80 |
| Ostf1 | −0.757739549 | 10.18398253 | 354.6784426 | 4.05846E−79 | 1.92968E−76 |
| Ifng | 1.390059422 | 9.686573599 | 352.8816351 | 9.99148E−79 | 4.61492E−76 |
| Hsph1 | 1.120114423 | 9.472352373 | 351.0872604 | 2.45683E−78 | 1.10325E−75 |
| Csrnp1 | 0.935572167 | 9.467362177 | 341.0348346 | 3.79755E−76 | 1.65922E−73 |
| Ppp1cc | −0.712400694 | 10.14035428 | 333.4083474 | 1.73963E−74 | 7.40074E−72 |
| Satb1 | −1.050886834 | 9.757129414 | 330.9907931 | 5.8478E−74 | 2.42399E−71 |
| Sub1 | −0.498310969 | 11.14885309 | 313.5062323 | 3.76209E−70 | 1.52045E−67 |
| Atf3 | 1.255366092 | 9.293173328 | 311.0704066 | 1.27657E−69 | 5.03343E−67 |
| Tnfrsf4 | −0.886369845 | 10.622478 | 308.5109707 | 4.60895E−69 | 1.77401E−66 |
| Dgat1 | −1.090343512 | 9.778876472 | 305.9824441 | 1.63848E−68 | 6.15991E−66 |
| Odc1 | −0.912868376 | 10.45380252 | 294.6839137 | 4.74244E−66 | 1.74241E−63 |
| Dnaja1 | 0.660328779 | 10.76356594 | 292.0198727 | 1.80488E−65 | 6.48395E−63 |
| Hmgb2 | −0.68462046 | 10.71173142 | 285.2036862 | 5.51645E−64 | 1.93867E−61 |
| Tnfrsf9 | −1.386299016 | 9.402138025 | 270.8266947 | 7.49438E−61 | 2.57775E−58 |
| Abi3 | 0.939528469 | 9.334614155 | 254.4801334 | 2.74009E−57 | 9.22838E−55 |
| Icos | −0.694823726 | 10.25651136 | 252.956149 | 5.88827E−57 | 1.94265E−54 |
| Lmnb1 | −0.796445565 | 9.781598082 | 235.4925917 | 3.78059E−53 | 1.22234E−50 |
| Tpm4 | −0.870737305 | 9.58004696 | 232.0124485 | 2.17005E−52 | 6.87863E−50 |
| Gabpb1 | −1.01277835 | 9.478116622 | 231.3253372 | 3.06417E−52 | 9.52602E−50 |
| Il21r | −0.709656805 | 10.19619458 | 230.7497394 | 4.09108E−52 | 1.24786E−49 |
| Actg1 | −0.385352565 | 12.09967582 | 227.9718392 | 1.65067E−51 | 4.9416E−49 |
| Nfkb2 | −0.872840303 | 9.521887809 | 227.4662535 | 2.12776E−51 | 6.25408E−49 |
| Tra2b | 0.566603683 | 9.814659783 | 225.7459621 | 5.04794E−51 | 1.45723E−48 |
| Nr4a1 | 0.760017624 | 9.629880779 | 225.0326612 | 7.22249E−51 | 2.0484E−48 |
| Actb | −0.686498084 | 13.40455375 | 224.189125 | 1.10323E−50 | 3.07497E−48 |
| Malat1 | 0.384658746 | 14.66845119 | 222.1869687 | 3.01551E−50 | 8.26249E−48 |
| Crem | −0.929344412 | 10.06723454 | 221.6786464 | 3.89255E−50 | 1.04878E−47 |
| Irf1 | 0.800988362 | 9.430944526 | 220.5435406 | 6.88374E−50 | 1.8243E−47 |
| S100a4 | −1.124176336 | 9.866850446 | 220.2298465 | 8.05839E−50 | 2.10116E−47 |
| Dnajb4 | 0.898452918 | 9.252159229 | 218.0567114 | 2.40034E−49 | 6.15935E−47 |
| Eif4a2 | 0.587188346 | 9.723691704 | 215.5267639 | 8.55372E−49 | 2.16062E−46 |
| Lgals1 | −0.544772962 | 11.62803108 | 210.6664274 | 9.82814E−48 | 2.44433E−45 |
| Vps37b | −0.567998001 | 10.60462455 | 208.2553343 | 3.29997E−47 | 8.08292E−45 |
| Arpc2 | −0.339096705 | 10.79502771 | 198.9781559 | 3.48999E−45 | 8.42077E−43 |
| Ldha | −0.477227524 | 10.356303 | 198.1191431 | 5.37386E−45 | 1.27756E−42 |
| Slc38a2 | 0.6945798 | 9.471310328 | 196.4081687 | 1.26965E−44 | 2.97467E−42 |
| Rhoh | 0.680272061 | 9.50146616 | 195.902854 | 1.63669E−44 | 3.77982E−42 |
| Kcnk5 | 0.865065562 | 9.212048811 | 195.5640523 | 1.94048E−44 | 4.41827E−42 |
| Cd47 | −0.469147919 | 10.23587831 | 193.5887493 | 5.23627E−44 | 1.17569E−41 |
| Cd28 | 0.462328972 | 10.37935338 | 193.379503 | 5.8169E−44 | 1.28817E−41 |
| Hspe1 | 0.62125485 | 10.275394 | 192.4921794 | 9.08574E−44 | 1.98487E−41 |
| Tnfrsf18 | −0.597638271 | 10.06784994 | 192.2396956 | 1.0315E−43 | 2.22336E−41 |
| Gadd45g | 1.091498475 | 9.253008237 | 191.6702361 | 1.37329E−43 | 2.92114E−41 |
| Sh2d2a | −0.665095634 | 9.846350864 | 191.0225578 | 1.90166E−43 | 3.99249E−41 |
| Cd2 | −0.492480832 | 10.31976175 | 186.0579796 | 2.30587E−42 | 4.77907E−40 |
| Ccdc117 | 0.815294707 | 9.229125118 | 181.8435023 | 1.91832E−41 | 3.9255E−39 |
| mt-Nd1 | −0.358612694 | 11.14922829 | 179.4352069 | 6.43782E−41 | 1.30092E−38 |
| Prelid1 | −0.628281747 | 9.693861972 | 176.724491 | 2.51555E−40 | 5.02053E−38 |
| Ptp4a2 | −0.517215133 | 9.957507214 | 175.4580103 | 4.75543E−40 | 9.37515E−38 |
| Btg2 | 0.440636277 | 10.53755263 | 170.6805646 | 5.25444E−39 | 1.02341E−36 |
| Rplp0 | −0.21033764 | 12.38791457 | 169.8702747 | 7.89769E−39 | 1.51993E−36 |
| Rora | −0.613089505 | 9.982326095 | 165.5083791 | 7.08364E−38 | 1.34723E−35 |
| Cytip | −0.464571312 | 10.18852324 | 164.5920026 | 1.12315E−37 | 2.11126E−35 |
| Ywhae | −0.599038836 | 9.677391704 | 163.9902312 | 1.52018E−37 | 2.82474E−35 |
| Dusp2 | 0.836057215 | 9.446119208 | 163.7671751 | 1.70068E−37 | 3.12424E−35 |
| Gm8797 | 0.673519794 | 9.41752959 | 163.683435 | 1.77385E−37 | 3.22203E−35 |
| Txnip | 0.67807196 | 9.461611552 | 163.1794609 | 2.28568E−37 | 4.06048E−35 |
| Crip1 | −0.565568902 | 11.66680939 | 163.1805573 | 2.28442E−37 | 4.06048E−35 |
| Zeb1 | −0.876726155 | 9.368997506 | 162.7540564 | 2.83107E−37 | 4.97469E−35 |
| Xbp1 | −0.711218017 | 9.544485173 | 161.4373091 | 5.49063E−37 | 9.54425E−35 |
| Orai1 | −0.579250032 | 9.884591795 | 160.2159281 | 1.01501E−36 | 1.7456E−34 |
| Kdm6b | −0.863501587 | 9.740079571 | 155.8597275 | 9.0849E−36 | 1.54596E−33 |
| Serpinb6b | −0.806046844 | 9.622270494 | 153.802115 | 2.55844E−35 | 4.3083E−33 |
| Rps27l | −0.691596722 | 9.542736211 | 153.7031579 | 2.68906E−35 | 4.48159E−33 |
| S100a6 | −0.604109019 | 10.99650705 | 153.6377922 | 2.77898E−35 | 4.58419E−33 |
| Rbm3 | −0.359226464 | 10.73120229 | 152.3012466 | 5.44486E−35 | 8.89107E−33 |
| Pnrc1 | 0.353791235 | 10.57762445 | 151.8347313 | 6.88568E−35 | 1.11314E−32 |
| Osgin1 | 0.824202552 | 9.241883295 | 149.9615518 | 1.76752E−34 | 2.82908E−32 |
| Ppp1r16b | −0.763243765 | 9.488059972 | 148.3650029 | 3.9477E−34 | 6.25671E−32 |
| Jund | 0.404038488 | 11.83352068 | 147.7973495 | 5.25327E−34 | 8.24508E−32 |
| Dnajb6 | −0.461679658 | 10.02357702 | 146.7975356 | 8.68943E−34 | 1.3507E−31 |
| Mns1 | 0.755135013 | 9.25079002 | 146.6385192 | 9.41355E−34 | 1.44933E−31 |
| Rhob | 0.74975205 | 9.186759187 | 145.176197 | 1.96535E−33 | 2.99735E−31 |
| Rps26 | −0.284540248 | 10.99419139 | 142.8574902 | 6.31524E−33 | 9.54133E−31 |
| Amica1 | 0.785928661 | 9.335185757 | 138.7221207 | 5.066E−32 | 7.58305E−30 |
| Cd3g | 0.261967409 | 11.10926437 | 138.1653066 | 6.70559E−32 | 9.94519E−30 |
| Maff | 0.743744556 | 9.305178125 | 136.9026641 | 1.26643E−31 | 1.8612E−29 |
| Bcl2a1d | −0.556400874 | 9.947971478 | 135.9736136 | 2.022E−31 | 2.92191E−29 |
| Cxcl10 | 1.022791565 | 9.358380139 | 135.9713164 | 2.02434E−31 | 2.92191E−29 |
| mt-Co2 | −0.245307237 | 11.3867395 | 134.1830298 | 4.9824E−31 | 7.12791E−29 |
| Ndufa4 | −0.533333906 | 9.735537154 | 132.5007031 | 1.16265E−30 | 1.64873E−28 |
| Got1 | −0.718746583 | 9.85597876 | 130.9493044 | 2.54011E−30 | 3.57073E−28 |
| mt-Co1 | −0.214636506 | 12.2154689 | 130.618136 | 3.00128E−30 | 4.18265E−28 |
| Gch1 | −0.741103157 | 9.459479195 | 128.8681559 | 7.24762E−30 | 9.96255E−28 |
| Stmn1 | −1.367904009 | 9.430748828 | 128.8615105 | 7.27193E−30 | 9.96255E−28 |
| Serpinb1a | −1.211789733 | 9.512634009 | 128.4498585 | 8.94794E−30 | 1.21557E−27 |
| Zfp3612 | 0.548721708 | 9.813907288 | 127.2191653 | 1.6635E−29 | 2.24102E−27 |
| Samsn1 | −0.60534197 | 9.795514419 | 124.3876645 | 6.9293E−29 | 9.25777E−27 |
| Cacybp | 0.534106834 | 9.501045641 | 123.654719 | 1.00256E−28 | 1.32847E−26 |
| Bcl11b | 0.557187574 | 9.518363932 | 123.2267919 | 1.24387E−28 | 1.63483E−26 |
| Nab1 | −0.638659495 | 9.481607755 | 122.2066184 | 2.08007E−28 | 2.71181E−26 |
| Eif4a1 | −0.326967555 | 10.43982044 | 119.198664 | 9.47487E−28 | 1.22537E−25 |
| Ctsw | 0.420268747 | 9.943491723 | 118.6678023 | 1.23823E−27 | 1.58867E−25 |
| Manf | −0.503781652 | 9.719597578 | 116.9764749 | 2.90493E−27 | 3.69772E−25 |
| Sit1 | 0.594504365 | 9.400129387 | 116.683346 | 3.36762E−27 | 4.2532E−25 |
| Ahr | 0.665620939 | 9.32837131 | 116.5385822 | 3.62263E−27 | 4.5398E−25 |
| Dnaja4 | 0.702154245 | 9.306294868 | 116.2943522 | 4.09737E−27 | 5.09524E−25 |
| Rasgrp1 | 0.54719873 | 9.4876411 | 115.5982599 | 5.82026E−27 | 7.18247E−25 |
| Rpl28 | −0.277273945 | 10.72583795 | 114.4206894 | 1.05399E−26 | 1.29081E−24 |
| Tnf | 0.657378601 | 9.19510683 | 113.7154721 | 1.50415E−26 | 1.82828E−24 |
| Map2k3 | −0.639195811 | 9.403893144 | 112.180806 | 3.26167E−26 | 3.93493E−24 |
| Bcl2a1b | −0.401042562 | 10.39223313 | 111.0495442 | 5.77103E−26 | 6.9107E−24 |
| mt-Co3 | −0.206451547 | 12.09928666 | 110.9099873 | 6.19193E−26 | 7.3602E−24 |
| Ddit3 | 0.600713605 | 9.375996145 | 110.3339779 | 8.27972E−26 | 9.77007E−24 |
| Arpc5 | −0.375095913 | 10.09994462 | 110.1520132 | 9.07572E−26 | 1.06317E−23 |
| Anxa2 | −0.551214664 | 9.842703511 | 109.8227857 | 1.07155E−25 | 1.24623E−23 |
| Tbx21 | 0.633815107 | 9.277654988 | 107.7502756 | 3.0487E−25 | 3.52038E−23 |
| Hspa5 | −0.331253095 | 10.97868219 | 106.0074139 | 7.34599E−25 | 8.42235E−23 |
| Gm26740 | −0.5631075 | 9.658905522 | 105.6525046 | 8.78684E−25 | 1.00034E−22 |
| Hspd1 | 0.522874198 | 9.781160578 | 105.5573721 | 9.21897E−25 | 1.0422E−22 |
| Egr1 | 0.693243113 | 9.204622648 | 105.294982 | 1.05242E−24 | 1.18149E−22 |
| Arpc3 | −0.300445815 | 10.54021802 | 104.3124691 | 1.72798E−24 | 1.92652E−22 |
| Slc25a5 | −0.317657126 | 10.39410861 | 102.9141475 | 3.49991E−24 | 3.87531E−22 |
| Slc25a19 | −0.671198564 | 9.304164557 | 102.4999831 | 4.31371E−24 | 4.74391E−22 |
| Arf5 | −0.355950623 | 9.981776324 | 102.4262272 | 4.47735E−24 | 4.89059E−22 |
| P2ry10 | −0.542627996 | 9.588148813 | 100.0926173 | 1.45435E−23 | 1.57792E−21 |
| Tiparp | 0.622859048 | 9.253508946 | 99.43211855 | 2.03003E−23 | 2.18784E−21 |
| Dapk2 | 0.629735843 | 9.267212724 | 99.37047465 | 2.09422E−23 | 2.24206E−21 |
| Srgn | −0.248081982 | 11.37107818 | 98.58322616 | 3.11648E−23 | 3.31454E−21 |
| S1pr4 | −0.599660571 | 9.466039302 | 98.42214907 | 3.38057E−23 | 3.57191E−21 |
| Prdx1 | −0.402768256 | 9.945248275 | 98.10603607 | 3.96568E−23 | 4.16294E−21 |
| Mif4gd | −0.554622287 | 9.488410444 | 97.43644609 | 5.56129E−23 | 5.80025E−21 |
| Pyhin1 | 0.625926041 | 9.241802634 | 97.31544236 | 5.91175E−23 | 6.12624E−21 |
| Atp2b1 | −0.577740061 | 9.529038489 | 96.38102805 | 9.4771E−23 | 9.75839E−21 |
| Klf4 | 0.785414982 | 9.240042515 | 95.6473965 | 1.3728E−22 | 1.4046E−20 |
| Kdm2b | −0.590418174 | 9.446725658 | 95.06884645 | 1.83878E−22 | 1.86954E−20 |
| Gpr34 | 0.643868069 | 9.256515481 | 94.58899127 | 2.34318E−22 | 2.36749E−20 |
| Ddx5 | 0.222201788 | 11.19915484 | 94.33581499 | 2.66288E−22 | 2.6738E−20 |
| Dedd2 | 0.548833108 | 9.289954935 | 93.22117023 | 4.67652E−22 | 4.66671E−20 |
| Gngt2 | 0.563130652 | 9.363419251 | 92.71645706 | 6.03496E−22 | 5.98535E−20 |
| Ptger4 | 0.593740113 | 9.344615159 | 92.63473731 | 6.28938E−22 | 6.19964E−20 |
| Rap1b | −0.323870206 | 10.11242022 | 92.0761552 | 8.34043E−22 | 8.1716E−20 |
| Ndfip1 | −0.453916191 | 9.658016099 | 91.91248652 | 9.05956E−22 | 8.8227E−20 |
| Pmaip1 | 0.569597424 | 9.419279032 | 91.40457314 | 1.17106E−21 | 1.13361E−19 |
| Ass1 | −0.570778569 | 9.534401383 | 91.02169791 | 1.42105E−21 | 1.36742E−19 |
| mt-Nd4 | −0.283267739 | 10.50324563 | 89.83638465 | 2.58693E−21 | 2.47457E−19 |
| Stat4 | −0.539139242 | 9.506546131 | 89.82057516 | 2.60768E−21 | 2.47975E−19 |
| Zfand2a | 0.571863902 | 9.203483651 | 89.75384762 | 2.69713E−21 | 2.54982E−19 |
| Peak1 | 0.543559655 | 9.307893678 | 89.44490698 | 3.15298E−21 | 2.96343E−19 |
| Ube2c | −0.73303176 | 9.233537011 | 89.11225779 | 3.73032E−21 | 3.4858E−19 |
| Klc1 | −0.689120968 | 9.658425271 | 89.02069137 | 3.90705E−21 | 3.62996E−19 |
| Ccnb2 | −0.590326734 | 9.212369085 | 88.83145927 | 4.29924E−21 | 3.97152E−19 |
| Kras | −0.549993886 | 9.430506668 | 88.81040425 | 4.34525E−21 | 3.99121E−19 |
| Atf4 | 0.472417228 | 9.430489091 | 88.68127167 | 4.63836E−21 | 4.23637E−19 |
| Tbc1d10c | 0.461521425 | 9.522565087 | 88.25782723 | 5.74554E−21 | 5.21812E−19 |
| S1pr1 | −0.676389037 | 9.290347327 | 87.8844838 | 6.93909E−21 | 6.26689E−19 |
| Oaz1 | −0.179343623 | 11.23129158 | 87.66043355 | 7.77135E−21 | 6.97954E−19 |
| Mkrn1 | −0.435496851 | 9.625707011 | 86.99298233 | 1.08907E−20 | 9.72701E−19 |
| Mpc1 | −0.482130101 | 9.498929683 | 86.81722357 | 1.19028E−20 | 1.05726E−18 |
| Cwc15 | −0.440746307 | 9.591066965 | 86.59722366 | 1.33034E−20 | 1.1752E−18 |
| Yeats4 | −0.576896023 | 9.335828716 | 86.0310623 | 1.77131E−20 | 1.55625E−18 |
| Zc3hav1 | −0.453828247 | 9.665187247 | 85.82752191 | 1.96335E−20 | 1.71565E−18 |
| Mxi1 | −0.615229665 | 9.308272849 | 85.48875372 | 2.33023E−20 | 2.0253E−18 |
| Gna13 | −0.394257142 | 9.985132901 | 85.39771314 | 2.44003E−20 | 2.10938E−18 |
| Eno1 | −0.376092563 | 9.94247872 | 85.37363547 | 2.46992E−20 | 2.12387E−18 |
| Metap2 | −0.532246668 | 9.437215686 | 84.64093112 | 3.57781E−20 | 3.06026E−18 |
| Cd53 | −0.276565437 | 10.54705579 | 84.11975064 | 4.65695E−20 | 3.96233E−18 |
| H3f3a | −0.262916696 | 10.34162816 | 84.03680502 | 4.85649E−20 | 4.11047E−18 |
| Chmp4b | −0.386119126 | 9.755857713 | 83.79332771 | 5.49299E−20 | 4.62498E−18 |
| Rgcc | 0.655205768 | 9.563495027 | 82.72779326 | 9.41678E−20 | 7.88765E−18 |
| Fam189b | 0.494624586 | 9.381139083 | 82.68518295 | 9.62198E−20 | 8.01799E−18 |
| Srp9 | −0.371205013 | 9.749456535 | 82.04588879 | 1.32963E−19 | 1.1023E−17 |
| Myl12a | −0.241036233 | 10.67854662 | 81.89353345 | 1.43618E−19 | 1.18456E−17 |
| Ube2d2a | −0.380758113 | 9.705617285 | 81.87867272 | 1.44702E−19 | 1.18744E−17 |
| Rcbtb2 | 0.554113844 | 9.398942766 | 81.83643178 | 1.47828E−19 | 1.20697E−17 |
| C1d | −0.55506624 | 9.33962429 | 81.52340884 | 1.73198E−19 | 1.40699E−17 |
| Cenpa | −0.644600095 | 9.394732697 | 81.06499933 | 2.18414E−19 | 1.76544E−17 |
| Rps2 | −0.182975709 | 11.63596206 | 80.5615055 | 2.81796E−19 | 2.26642E−17 |
| Rab2a | −0.405486929 | 9.638428497 | 80.08770238 | 3.58155E−19 | 2.8663E−17 |
| Rps17 | −0.253525207 | 10.61527996 | 79.99091046 | 3.76136E−19 | 2.99538E−17 |
| Crbn | −0.45331621 | 9.708110897 | 79.55964126 | 4.67886E−19 | 3.70777E−17 |
| Evl | 0.449484781 | 9.48202136 | 79.48943941 | 4.8481E−19 | 3.82314E−17 |
| Atp1b3 | −0.525675487 | 9.413041736 | 79.31700201 | 5.29025E−19 | 4.15156E−17 |
| Birc5 | −0.803153176 | 9.251717708 | 78.81955099 | 6.80503E−19 | 5.3145E−17 |
| Pelo | 0.534389282 | 9.234977676 | 78.7693748 | 6.98008E−19 | 5.425E−17 |
| Ube2b | −0.340873439 | 9.912296976 | 78.67640507 | 7.31642E−19 | 5.6592E−17 |
| Slc25a3 | −0.335596538 | 9.94973675 | 78.52605368 | 7.89501E−19 | 6.07765E−17 |
| Gimap9 | 0.414105757 | 9.580964503 | 77.89866942 | 1.08464E−18 | 8.31012E−17 |
| Gbp8 | 0.519003936 | 9.225073835 | 77.70588153 | 1.19585E−18 | 9.1189E−17 |
| Sash3 | 0.526717574 | 9.280790532 | 76.92431049 | 1.77637E−18 | 1.34821E−16 |
| Nfkb1 | −0.482750495 | 9.537095283 | 76.9019338 | 1.79661E−18 | 1.3572E−16 |
| Tgfb1 | −0.436908782 | 9.751104871 | 76.52399621 | 2.17553E−18 | 1.6358E−16 |
| Gm19585 | −0.589333363 | 9.333697403 | 76.49300801 | 2.20994E−18 | 1.65398E−16 |
| 2810417H13Rik | −1.137160497 | 9.337791148 | 76.28397891 | 2.45669E−18 | 1.83018E−16 |
| Erh | −0.391194693 | 9.741401362 | 75.75485582 | 3.2116E−18 | 2.3816E−16 |
| Nabp1 | −0.474933711 | 9.632410427 | 75.07324761 | 4.53571E−18 | 3.34814E−16 |
| H3f3b | −0.183055632 | 12.3442719 | 75.06348853 | 4.55818E−18 | 3.34944E−16 |
| Tra2a | 0.437013456 | 9.420550179 | 74.87618174 | 5.0118E−18 | 3.6661E−16 |
| Rpl4 | −0.145002236 | 11.68907687 | 74.81745426 | 5.16312E−18 | 3.75978E−16 |
| Reep5 | −0.410263139 | 9.63723477 | 74.70029247 | 5.4788E−18 | 3.97176E−16 |
| ler2 | 0.372614919 | 9.828501111 | 74.64201981 | 5.64293E−18 | 4.07248E−16 |
| Abcb1b | −0.617002181 | 9.297095714 | 74.45737621 | 6.19617E−18 | 4.45188E−16 |
| Atp5a1 | −0.343731345 | 9.817876037 | 74.34844531 | 6.54768E−18 | 4.68362E−16 |
| Pfn1 | −0.208967825 | 12.14558871 | 74.16052907 | 7.20159E−18 | 5.12868E−16 |
| Madd | −0.529984212 | 9.387287048 | 74.00633058 | 7.78669E−18 | 5.52104E−16 |
| Tomm20 | −0.285577806 | 10.023749 | 73.94492556 | 8.03272E−18 | 5.67061E−16 |
| Cmas | −0.523719597 | 9.365634653 | 73.92293145 | 8.12272E−18 | 5.70921E−16 |
| Cdca3 | −0.440059827 | 9.19681312 | 73.80929833 | 8.60403E−18 | 6.02133E−16 |
| Cd226 | 0.541531319 | 9.268561012 | 73.70228655 | 9.08336E−18 | 6.32938E−16 |
| Stk4 | −0.531488797 | 9.393563986 | 73.65355009 | 9.31042E−18 | 6.45976E−16 |
| Cript | −0.418219478 | 9.556480747 | 73.60266337 | 9.55357E−18 | 6.60013E−16 |
| Lztfl1 | 0.516188841 | 9.420902233 | 73.17831763 | 1.1845E−17 | 8.14834E−16 |
| Cox5b | −0.29984058 | 10.04351529 | 72.98093175 | 1.30909E−17 | 8.96723E−16 |
| Snrpe | −0.339808137 | 9.827473175 | 72.71911464 | 1.49479E−17 | 1.01961E−15 |
| Rps3a1 | −0.144846919 | 11.67403179 | 72.62804541 | 1.56539E−17 | 1.06328E−15 |
| Gpx4 | −0.322618499 | 10.07619191 | 71.72461952 | 2.47426E−17 | 1.67359E−15 |
| Lcp1 | 0.267873223 | 10.29111652 | 70.77546944 | 4.00283E−17 | 2.69624E−15 |
| Gramd1a | 0.428949158 | 9.45882935 | 70.50743203 | 4.58536E−17 | 3.0758E−15 |
| Rpl22l1 | −0.227855341 | 10.87577052 | 70.42828761 | 4.77306E−17 | 3.18848E−15 |
| Carnmt1 | −0.533340181 | 9.382599136 | 70.14896737 | 5.49908E−17 | 3.65836E−15 |
| Fam78a | 0.483182295 | 9.208922936 | 69.96525619 | 6.03583E−17 | 3.99898E−15 |
| Itk | −0.384569882 | 9.814381707 | 69.34499645 | 8.26621E−17 | 5.45435E−15 |
| Sirt7 | 0.451348242 | 9.346429512 | 69.25139271 | 8.66797E−17 | 5.6962E−15 |
| Lpar6 | 0.50184775 | 9.25603568 | 69.13687452 | 9.18617E−17 | 6.01229E−15 |
| Podnl1 | 0.465684375 | 9.399826176 | 69.07032239 | 9.50144E−17 | 6.19356E−15 |
| Cd27 | −0.693527396 | 9.342790688 | 69.00506198 | 9.8211E−17 | 6.37622E−15 |
| Il1r2 | −0.769598989 | 9.256734057 | 68.4351569 | 1.31119E−16 | 8.47868E−15 |
| Rpl31 | −0.198288808 | 10.87174216 | 68.22727856 | 1.45696E−16 | 9.38377E−15 |
| Ube2a | −0.430232394 | 9.476224302 | 68.15744891 | 1.50948E−16 | 9.68345E−15 |
| Il2rg | 0.284318296 | 10.13321138 | 68.10803166 | 1.54779E−16 | 9.88995E−15 |
| Btf3 | −0.188936964 | 10.71562669 | 67.84995712 | 1.76422E−16 | 1.12285E−14 |
| Flna | −0.543132079 | 9.271089863 | 67.71229144 | 1.89181E−16 | 1.19933E−14 |
| Ostc | −0.417712022 | 9.507638046 | 67.39567345 | 2.22136E−16 | 1.40275E−14 |
| Nap1l1 | −0.446817289 | 9.497843766 | 67.25802126 | 2.382E−16 | 1.49834E−14 |
| Gm26532 | 0.539119111 | 9.298825204 | 67.17554702 | 2.48376E−16 | 1.5563E−14 |
| Rassf5 | 0.40289647 | 9.456960458 | 67.14089843 | 2.5278E−16 | 1.57598E−14 |
| Psmd6 | −0.511849794 | 9.309065396 | 67.13554255 | 2.53467E−16 | 1.57598E−14 |
| Slc39a1 | −0.517971835 | 9.215123658 | 66.93618839 | 2.80439E−16 | 1.737E−14 |
| Sipa1 | 0.445171877 | 9.377580607 | 66.78277931 | 3.03135E−16 | 1.87041E−14 |
| Rpl24 | −0.138569497 | 11.77231724 | 66.27823268 | 3.91559E−16 | 2.40682E−14 |
| Plk3 | 0.492663873 | 9.37620264 | 66.21240355 | 4.04857E−16 | 2.47913E−14 |
| 1500009L16Rik | −0.568387028 | 9.247088485 | 65.79956575 | 4.99192E−16 | 3.04526E−14 |
| Arf4 | −0.322744466 | 9.97486799 | 65.76286864 | 5.08574E−16 | 3.09083E−14 |
| Ccr7 | −0.852512495 | 9.2731796 | 65.71215821 | 5.2183E−16 | 3.15507E−14 |
| Hmgb1 | −0.289258843 | 10.29717658 | 65.70756036 | 5.23048E−16 | 3.15507E−14 |
| Psmb5 | −0.408420536 | 9.522708713 | 65.65722349 | 5.36579E−16 | 3.22466E−14 |
| Zfp3611 | 0.437347092 | 9.536424792 | 65.53388802 | 5.71232E−16 | 3.4202E−14 |
| Rsrp1 | 0.294708181 | 10.00255998 | 65.36956236 | 6.20905E−16 | 3.70389E−14 |
| Ccnd3 | 0.392403552 | 9.527397103 | 64.82584356 | 8.1819E−16 | 4.86282E−14 |
| Etf1 | −0.44463723 | 9.430618507 | 64.09079524 | 1.18815E−15 | 7.03578E−14 |
| Rpl37a | 0.122987072 | 12.61564076 | 63.93640264 | 1.28501E−15 | 7.58157E−14 |
| Gapdh | −0.237914704 | 10.5624293 | 63.49863181 | 1.60478E−15 | 9.43379E−14 |
| St3gal6 | 0.467788849 | 9.290007328 | 63.39446236 | 1.69193E−15 | 9.91005E−14 |
| Stat3 | −0.427360634 | 9.627931004 | 63.34309522 | 1.73663E−15 | 1.01352E−13 |
| Capza2 | −0.336100212 | 9.686699536 | 63.08839131 | 1.97635E−15 | 1.14927E−13 |
| Aim1 | −0.518130757 | 9.317849014 | 62.96031012 | 2.10914E−15 | 1.22209E−13 |
| 4930503L19Rik | 0.477664395 | 9.217221064 | 62.90849567 | 2.16536E−15 | 1.25019E−13 |
| Ptpn22 | −0.320448302 | 10.0931864 | 62.87594088 | 2.20145E−15 | 1.2665E−13 |
| Isy1 | −0.489261775 | 9.388853979 | 62.86177423 | 2.21734E−15 | 1.27112E−13 |
| Dcun1d5 | −0.394885964 | 9.525965055 | 62.63746655 | 2.48481E−15 | 1.41942E−13 |
| Sys1 | −0.391713519 | 9.549646283 | 62.46696151 | 2.70952E−15 | 1.54233E−13 |
| Cox6a1 | −0.247773288 | 10.15026993 | 62.43173845 | 2.75842E−15 | 1.56465E−13 |
| Hif1a | −0.36303621 | 9.774226901 | 62.23603398 | 3.04662E−15 | 1.72209E−13 |
| Dennd4a | −0.506038575 | 9.353170513 | 61.80891426 | 3.78457E−15 | 2.13176E−13 |
| Ermn | −0.670779521 | 9.304666665 | 61.78992983 | 3.82124E−15 | 2.14493E−13 |
| Pglyrp1 | −0.581505876 | 9.331204063 | 61.76551296 | 3.86891E−15 | 2.16418E−13 |
| Smad7 | 0.432811456 | 9.374760348 | 61.6967973 | 4.00631E−15 | 2.23331E−13 |
| Emp3 | −0.353830378 | 9.864716413 | 61.4528883 | 4.53465E−15 | 2.51914E−13 |
| Mif | −0.336814609 | 9.843573442 | 61.40913837 | 4.63654E−15 | 2.56693E−13 |
| Ncl | −0.277240909 | 10.07288653 | 61.28557928 | 4.93683E−15 | 2.72385E−13 |
| Tax1bp1 | −0.346224474 | 9.700820891 | 61.18575971 | 5.19358E−15 | 2.85576E−13 |
| Gm11808 | 0.267683291 | 10.17414513 | 61.10326802 | 5.41582E−15 | 2.96787E−13 |
| Tnip1 | −0.466236205 | 9.396423421 | 61.03989719 | 5.59298E−15 | 3.0546E−13 |
| Il2rb | −0.377545771 | 9.646395321 | 60.97710191 | 5.77425E−15 | 3.14298E−13 |
| Selk | −0.25836491 | 10.04778191 | 60.88064076 | 6.06423E−15 | 3.28974E−13 |
| mt-Atp6 | −0.143668358 | 12.47117091 | 60.56120241 | 7.1326E−15 | 3.85638E−13 |
| Rpl32 | −0.118637876 | 12.44744241 | 60.38463939 | 7.80194E−15 | 4.20421E−13 |
| Uap1 | −0.479451457 | 9.373416437 | 60.17826839 | 8.66436E−15 | 4.65342E−13 |
| B2m | 0.104190832 | 12.79985088 | 60.16460126 | 8.72473E−15 | 4.67033E−13 |
| Ubl3 | −0.402699619 | 9.565973366 | 59.28291636 | 1.36557E−14 | 7.28574E−13 |
| Cd160 | 0.664206821 | 9.334737973 | 59.26672523 | 1.37685E−14 | 7.32177E−13 |
| Nfkbid | 0.548241659 | 9.347248926 | 59.24549012 | 1.39179E−14 | 7.37694E−13 |
| Sdhb | −0.373471788 | 9.57477766 | 59.140164 | 1.46831E−14 | 7.75712E−13 |
| Cd40lg | 0.434709702 | 9.469009802 | 59.079041 | 1.51464E−14 | 7.97579E−13 |
| Ccl4 | 1.216184948 | 9.567139597 | 59.07002117 | 1.5216E−14 | 7.98642E−13 |
| Ifi203 | 0.397729022 | 9.466695884 | 59.02071419 | 1.56021E−14 | 8.16257E−13 |
| Hsp90b1 | −0.360458376 | 9.704110499 | 58.95054865 | 1.61685E−14 | 8.4316E−13 |
| Atp5f1 | −0.298472638 | 9.82228089 | 58.8189421 | 1.72869E−14 | 8.98585E−13 |
| Ms4a6b | 0.26638962 | 10.19482982 | 58.69772529 | 1.83854E−14 | 9.52623E−13 |
| Ell2 | −0.499117657 | 9.233861253 | 58.59765846 | 1.93447E−14 | 9.99124E−13 |
| Rps27 | 0.130528567 | 13.23482143 | 58.50543285 | 2.0273E−14 | 1.04374E−12 |
| Pin1 | −0.459464991 | 9.345121381 | 58.32632468 | 2.22053E−14 | 1.13959E−12 |
| Nmrk1 | −0.50895163 | 9.47064308 | 58.21551447 | 2.34919E−14 | 1.2018E−12 |
| Ccna2 | −0.39001523 | 9.193845605 | 58.12778027 | 2.45633E−14 | 1.25265E−12 |
| Ube2j2 | −0.376431055 | 9.513342518 | 58.03685007 | 2.57253E−14 | 1.30779E−12 |
| Rpl38 | 0.138662549 | 11.67460466 | 58.0220768 | 2.59193E−14 | 1.31351E−12 |
| Uqcrfs1 | −0.314965169 | 9.7148766 | 57.9988405 | 2.62272E−14 | 1.32497E−12 |
| Rasgrp4 | 0.436494297 | 9.196048428 | 57.85877003 | 2.81629E−14 | 1.41832E−12 |
| Gm4955 | 0.499775199 | 9.225196819 | 57.61657645 | 3.1853E−14 | 1.59918E−12 |
| Pik3r1 | −0.45075266 | 9.484798888 | 57.58402923 | 3.23844E−14 | 1.62083E−12 |
| Tuba4a | 0.431200046 | 9.389388654 | 56.87585494 | 4.64211E−14 | 2.31618E−12 |
| Smarce1 | −0.408252588 | 9.44121571 | 56.75737258 | 4.93039E−14 | 2.45245E−12 |
| Rac1 | −0.27734301 | 9.864108042 | 56.73130979 | 4.99617E−14 | 2.47755E−12 |
| Nfkbiz | 0.364141505 | 9.633378704 | 56.64467691 | 5.2212E−14 | 2.58122E−12 |
| Rpl7 | −0.145513172 | 11.2121183 | 56.47267357 | 5.69848E−14 | 2.80859E−12 |
| Tm2d2 | −0.487628577 | 9.29650158 | 56.43314473 | 5.8142E−14 | 2.84825E−12 |
| Prkar1a | −0.244526548 | 10.1098473 | 56.43460194 | 5.80989E−14 | 2.84825E−12 |
| Hn1 | −0.290730748 | 9.912834007 | 56.21069494 | 6.51064E−14 | 3.17979E−12 |
| Vimp | −0.385693308 | 9.528415566 | 56.17137881 | 6.64214E−14 | 3.23424E−12 |
| Il12rb1 | 0.457617355 | 9.249603528 | 56.07279355 | 6.98368E−14 | 3.39034E−12 |
| Sar1a | −0.38919719 | 9.487454256 | 55.83677354 | 7.87447E−14 | 3.81134E−12 |
| Rgs3 | 0.445767105 | 9.212312097 | 55.42399885 | 9.71432E−14 | 4.68781E−12 |
| H2afx | −0.53405617 | 9.351667018 | 55.39133439 | 9.87709E−14 | 4.75217E−12 |
| Fam46a | −0.593104785 | 9.504386119 | 54.85249022 | 1.29924E−13 | 6.2325E−12 |
| Unc50 | −0.463482394 | 9.291047169 | 54.8152367 | 1.3241E−13 | 6.33297E−12 |
| Psmb6 | −0.315898886 | 9.664543895 | 54.5840988 | 1.48936E−13 | 7.10235E−12 |
| Relb | −0.416261884 | 9.473543412 | 54.39215697 | 1.64217E−13 | 7.80803E−12 |
| Areg | −0.981677364 | 9.386135347 | 54.37400443 | 1.65741E−13 | 7.85739E−12 |
| Nusap1 | −0.478892551 | 9.207262276 | 54.2609206 | 1.75559E−13 | 8.29849E−12 |
| Dnajb11 | −0.399861357 | 9.443671568 | 54.13350774 | 1.8732E−13 | 8.82859E−12 |
| Rpl8 | −0.128862907 | 11.65232029 | 54.1236023 | 1.88266E−13 | 8.84742E−12 |
| Psmb4 | −0.368425685 | 9.512666637 | 54.07411222 | 1.93068E−13 | 9.04679E−12 |
| Pfdn5 | 0.183243053 | 10.55366333 | 53.80238941 | 2.21703E−13 | 1.03585E−11 |
| Pdia6 | −0.405605687 | 9.493421923 | 53.74006008 | 2.28849E−13 | 1.06616E−11 |
| Tdg | −0.465092218 | 9.267189123 | 53.56871386 | 2.49704E−13 | 1.15998E−11 |
| Rasgrp2 | −0.474109476 | 9.377109112 | 53.28818106 | 2.88034E−13 | 1.3342E−11 |
| Chordc1 | 0.43790998 | 9.243519132 | 53.22033495 | 2.98156E−13 | 1.37714E−11 |
| Eomes | −0.676427393 | 9.215474018 | 52.88113851 | 3.54356E−13 | 1.63206E−11 |
| Riok1 | −0.482947013 | 9.379980367 | 52.86480692 | 3.57315E−13 | 1.64101E−11 |
| Mrpl43 | −0.430748788 | 9.347336785 | 52.85289924 | 3.59488E−13 | 1.64631E−11 |
| B930036N10Rik | 0.476518913 | 9.189748854 | 52.78752614 | 3.71654E−13 | 1.69722E−11 |
| Snrpd1 | −0.387511242 | 9.480040942 | 52.74552695 | 3.79687E−13 | 1.72902E−11 |
| Rps27rt | 0.21726945 | 11.16206124 | 52.6596686 | 3.96653E−13 | 1.80121E−11 |
| Tnfrsf25 | −0.507674416 | 9.258889745 | 52.54427375 | 4.20657E−13 | 1.90486E−11 |
| Capzb | −0.237573376 | 10.15899344 | 52.46292909 | 4.38447E−13 | 1.97987E−11 |
| Cks1b | −0.578941254 | 9.261808188 | 52.11841755 | 5.22525E−13 | 2.34643E−11 |
| Eif1 | −0.13433723 | 11.85889558 | 52.12359736 | 5.21149E−13 | 2.34643E−11 |
| Psmb2 | −0.31086955 | 9.69395085 | 52.03727219 | 5.44571E−13 | 2.43865E−11 |
| Fnta | −0.448658046 | 9.225372599 | 51.97384086 | 5.6245E−13 | 2.51176E−11 |
| Cisd2 | −0.391653223 | 9.458300452 | 51.95684696 | 5.67339E−13 | 2.52661E−11 |
| Cirbp | −0.312038116 | 9.821586015 | 51.83115822 | 6.04843E−13 | 2.68623E−11 |
| Cdc25b | −0.45061542 | 9.220519401 | 51.78564824 | 6.19026E−13 | 2.74169E−11 |
| Ndufb9 | −0.334018339 | 9.600272915 | 51.60840143 | 6.7751E−13 | 2.99252E−11 |
| Rps8 | −0.135138035 | 11.5927656 | 51.58476284 | 6.85717E−13 | 3.02052E−11 |
| Tmem165 | −0.454708653 | 9.262410636 | 51.47792094 | 7.24068E−13 | 3.18079E−11 |
| Acot9 | −0.455587461 | 9.289681303 | 51.33930244 | 7.77043E−13 | 3.40425E−11 |
| Gna15 | −0.42915867 | 9.413846476 | 51.31381614 | 7.87197E−13 | 3.43941E−11 |
| Batf | −0.397304061 | 9.653588452 | 51.22533009 | 8.23491E−13 | 3.58829E−11 |
| Marcksl1 | −0.536219973 | 9.352514486 | 51.11147635 | 8.72666E−13 | 3.79234E−11 |
| Cd4 | 0.302355981 | 9.643165705 | 51.0304068 | 9.09462E−13 | 3.94165E−11 |
| Fosl2 | −0.454425467 | 9.531626337 | 50.9750422 | 9.35479E−13 | 4.04357E−11 |
| Bcl211 | −0.502971673 | 9.333332611 | 50.85441484 | 9.94773E−13 | 4.2884E−11 |
| mt-Cytb | −0.182059117 | 11.07879142 | 50.36934489 | 1.27368E−12 | 5.47616E−11 |

TABLE 3F
LPL−5 vs. All

| GeneSymbol | logFC | logCPM | LR | PValue | FDR |
|---|---|---|---|---|---|
| Tnfrsf4 | 1.093522489 | 10.62226106 | 635.4400281 | 3.2785E−140 | 5.3E−136 |
| Tgfb1 | 0.975322652 | 9.751061303 | 520.2631269 | 3.7093E−115 | 2.9982E−111 |
| Bcl2a1b | 0.780135087 | 10.39216426 | 514.3928564 | 7.022E−114 | 3.7839E−110 |
| Bcl2a1d | 0.89577233 | 9.947939732 | 466.5419171 | 1.8125E−103 | 7.3252E−100 |
| Rpl13a | −0.326086663 | 13.13152047 | 458.393353 | 1.0753E−101 | 3.47664E−98 |
| Furin | 1.083121158 | 10.3814011 | 438.3306291 | 2.49901E−97 | 6.73315E−94 |
| Fam110a | 1.071979962 | 9.494271745 | 431.7915513 | 6.62154E−96 | 1.38439E−92 |
| Srgn | 0.499629456 | 11.37085449 | 431.7236111 | 6.85087E−96 | 1.38439E−92 |
| Re | 1.088827341 | 9.441983899 | 426.4493209 | 9.63164E−95 | 1.73006E−91 |
| Ctla4 | 0.839377678 | 10.15303035 | 392.4986426 | 2.36551E−87 | 3.82408E−84 |
| Id2 | 0.802587697 | 10.64561455 | 386.6248147 | 4.49435E−86 | 6.60507E−83 |
| Mmd | 1.251992193 | 9.302839885 | 381.7863212 | 5.08222E−85 | 6.8466E−82 |
| Cxcr6 | 0.819984404 | 10.3899312 | 377.6613434 | 4.01909E−84 | 4.99789E−81 |
| Tnfrsf9 | 1.354848987 | 9.402019905 | 371.2362656 | 1.00698E−82 | 1.16278E−79 |
| Rplp0 | −0.336949142 | 12.38776356 | 370.2741409 | 1.63119E−82 | 1.75799E−79 |
| Areg | 2.120364333 | 9.386124375 | 352.1899639 | 1.41334E−78 | 1.42801E−75 |
| Rps18 | −0.319352603 | 12.47332519 | 347.6549277 | 1.37346E−77 | 1.30608E−74 |
| Lag3 | 1.647526397 | 9.272200366 | 346.4561421 | 2.50539E−77 | 2.25012E−74 |
| Sdf4 | 0.666738475 | 9.917375756 | 342.4166661 | 1.89919E−76 | 1.61591E−73 |
| Hspa5 | 0.561489025 | 10.97852902 | 331.228038 | 5.19183E−74 | 4.19656E−71 |
| Rpl32 | −0.302417146 | 12.44734872 | 330.2768468 | 8.36546E−74 | 6.43981E−71 |
| Ehd1 | 0.91198395 | 9.471470346 | 322.9608951 | 3.28068E−72 | 2.4107E−69 |
| Bhlhe40 | 0.560219792 | 10.72354665 | 318.8827651 | 2.53665E−71 | 1.78293E−68 |
| Penk | 2.37977032 | 9.250280046 | 318.735219 | 2.73149E−71 | 1.83989E−68 |
| Gadd 45b | 1.002640146 | 9.727336037 | 316.7675708 | 7.32834E−71 | 4.73879E−68 |
| Il21r | 0.742031608 | 10.19622389 | 314.7885445 | 1.97741E−70 | 1.2295E−67 |
| Stat3 | 0.825967681 | 9.627915766 | 310.4807593 | 1.71591E−69 | 1.02739E−66 |
| Dusp14 | 1.311493662 | 9.241607784 | 302.6348425 | 8.78489E−68 | 5.07202E−65 |
| Actb | −0.868840136 | 13.40497483 | 291.6698206 | 2.1514E−65 | 1.19929E−62 |
| Nmrk1 | 0.997099834 | 9.470696344 | 289.2668478 | 7.18297E−65 | 3.87066E−62 |
| Plscr1 | 0.963349071 | 9.521574474 | 285.8877732 | 3.91377E−64 | 2.04097E−61 |
| Rps19 | −0.329912744 | 12.57926811 | 282.5048755 | 2.13673E−63 | 1.07945E−60 |
| Rora | 0.701354189 | 9.982410898 | 271.7577505 | 4.69699E−61 | 2.30096E−58 |
| Klf2 | −1.613268488 | 9.960790759 | 267.4297392 | 4.12189E−60 | 1.95984E−57 |
| Capg | 0.78217281 | 9.809382216 | 267.1893548 | 4.65038E−60 | 2.14795E−57 |
| Rpl29 | −0.371532652 | 11.21426477 | 265.936218 | 8.72202E−60 | 3.91667E−57 |
| Junb | 0.563013782 | 11.89122367 | 260.4847643 | 1.34539E−58 | 5.87828E−56 |
| Odc1 | 0.760576176 | 10.45354943 | 252.8701509 | 6.14801E−57 | 2.61549E−54 |
| Tnfrsf18 | 0.61336575 | 10.06773107 | 249.7951843 | 2.878E−56 | 1.19297E−53 |
| Rps5 | −0.27022034 | 12.08886363 | 247.3691807 | 9.72734E−56 | 3.9313E−53 |
| Stx11 | 0.844497 | 9.44438744 | 243.9575398 | 5.3928E−55 | 2.12634E−52 |
| H2-D1 | 0.267762308 | 12.28190082 | 240.4816947 | 3.08798E−54 | 1.18858E−51 |
| Ostf1 | 0.551472516 | 10.1838504 | 238.6389317 | 7.78902E−54 | 2.92831E−51 |
| Ermn | 1.23629042 | 9.304608573 | 238.0447323 | 1.04966E−53 | 3.85654E−51 |
| Rpl18 | −0.274617166 | 11.80689186 | 230.9705444 | 3.66172E−52 | 1.31545E−49 |
| Rpl18a | −0.230919204 | 12.8427361 | 230.9237523 | 3.74877E−52 | 1.31745E−49 |
| Ece1 | 0.949006386 | 9.199067932 | 228.403278 | 1.32913E−51 | 4.57163E−49 |
| Gem | 1.164414567 | 9.368386927 | 225.2504315 | 6.47426E−51 | 2.18048E−48 |
| Rpl13 | −0.237763931 | 12.637725 | 222.4024543 | 2.7062E−50 | 8.92825E−48 |
| Fam46a | 1.075761036 | 9.504325402 | 218.6210206 | 1.80792E−49 | 5.84536E−47 |
| Rps11 | −0.249263758 | 12.26305424 | 217.0901119 | 3.90053E−49 | 1.23639E−46 |
| Cd82 | 0.515694291 | 10.12752586 | 216.2520302 | 5.94214E−49 | 1.84732E−46 |
| Ccl20 | 1.571767244 | 9.175986718 | 215.708926 | 7.8058E−49 | 2.38092E−46 |
| Rps6 | −0.236751525 | 12.20838346 | 214.0506452 | 1.79543E−48 | 5.37499E−46 |
| Tnfsf11 | 1.000729109 | 9.5470138 | 208.7598636 | 2.56113E−47 | 7.52787E−45 |
| Rpl10a | −0.334028709 | 11.1421834 | 206.6183549 | 7.51057E−47 | 2.16814E−44 |
| Rps4x | −0.25351799 | 11.84794571 | 203.2452547 | 4.08952E−46 | 1.15985E−43 |
| H2-K1 | 0.310569301 | 11.6099805 | 199.5324875 | 2.64152E−45 | 7.36256E−43 |
| Gramd3 | −0.754266192 | 10.20642813 | 194.9036165 | 2.70425E−44 | 7.40965E−42 |
| Vgll4 | 0.685758581 | 9.732056241 | 192.3735559 | 9.64387E−44 | 2.59838E−41 |
| Rps10 | −0.298690801 | 11.22180355 | 192.3216417 | 9.8988E−44 | 2.62334E−41 |
| Zeb2 | 1.004261208 | 9.266619145 | 189.0385742 | 5.15457E−43 | 1.34401E−40 |
| Rps7 | −0.249396851 | 11.94311863 | 188.1593636 | 8.01885E−43 | 2.05766E−40 |
| Rpl14 | −0.247313373 | 11.87703907 | 187.7451879 | 9.87468E−43 | 2.49428E−40 |
| Rps16 | −0.223372261 | 12.35139052 | 184.4010715 | 5.30335E−42 | 1.31898E−39 |
| B2m | 0.192848994 | 12.79982102 | 184.2318403 | 5.77425E−42 | 1.41434E−39 |
| Rps3 | −0.216536407 | 11.96705851 | 183.2730085 | 9.35028E−42 | 2.25607E−39 |
| Rpl8 | −0.253480179 | 11.65218841 | 179.8911834 | 5.11893E−41 | 1.21695E−38 |
| Foxp3 | 0.625482423 | 9.163304427 | 179.8254281 | 5.29099E−41 | 1.23962E−38 |
| H3f3b | 0.284663226 | 12.34435911 | 173.7735014 | 1.1093E−39 | 2.56184E−37 |
| Timp2 | 0.802832395 | 9.295781647 | 172.1120915 | 2.55788E−39 | 5.82405E−37 |
| Ikzf2 | 1.073873069 | 9.298841109 | 170.9085501 | 4.68525E−39 | 1.05197E−36 |
| Ddx5 | 0.316171351 | 11.19923668 | 169.5222404 | 9.40838E−39 | 2.0835E−36 |
| Ly6e | 0.358526245 | 10.76648585 | 169.4937633 | 9.54409E−39 | 2.085E−36 |
| Cdkn1a | 0.806219522 | 9.693178201 | 169.0570774 | 1.18881E−38 | 2.56245E−36 |
| Trps1 | 0.845530208 | 9.247854818 | 163.5175317 | 1.92824E−37 | 4.10156E−35 |
| Orai1 | 0.526678565 | 9.884604613 | 162.1675472 | 3.80264E−37 | 7.98356E−35 |
| Tmsb10 | −0.244760928 | 12.94245948 | 160.4130676 | 9.19176E−37 | 1.90505E−34 |
| Rpsa | −0.258005375 | 11.60010538 | 160.3539494 | 9.46924E−37 | 1.93772E−34 |
| Zfp36l2 | −0.788376864 | 9.814128538 | 159.4437221 | 1.49689E−36 | 3.02484E−34 |
| Klf3 | −0.990105413 | 9.422133449 | 159.1328844 | 1.75027E−36 | 3.4932E−34 |
| Itm2b | 0.315828579 | 10.97659894 | 158.3293848 | 2.62222E−36 | 5.16961E−34 |
| Marcksl1 | 0.883574181 | 9.352455125 | 155.3767044 | 1.15844E−35 | 2.2563E−33 |
| Ndfip1 | 0.526351822 | 9.657958285 | 154.7210113 | 1.61125E−35 | 3.10088E−33 |
| Rps28 | −0.295632581 | 11.7074328 | 154.5592024 | 1.74792E−35 | 3.32434E−33 |
| Tnfsf8 | 0.870087777 | 10.01785574 | 153.9305856 | 2.39827E−35 | 4.5082E−33 |
| Rpl35 | −0.315995326 | 11.18856979 | 153.1436099 | 3.56359E−35 | 6.62172E−33 |
| Nfkb1 | 0.606837763 | 9.537083442 | 152.6546593 | 4.55771E−35 | 8.37273E−33 |
| Rbpj | 0.680460617 | 9.38517504 | 152.5848125 | 4.72076E−35 | 8.57482E−33 |
| Tubb5 | −0.744906297 | 10.00307441 | 152.0091079 | 6.30718E−35 | 1.13291E−32 |
| S1pr1 | −0.952521601 | 9.290338881 | 150.4433721 | 1.38692E−34 | 2.46383E−32 |
| Il2rb | 0.537577161 | 9.646357284 | 148.867921 | 3.06486E−34 | 5.3855E−32 |
| Litaf | 0.754556108 | 9.445062895 | 147.9067642 | 4.97178E−34 | 8.64235E−32 |
| Cd3g | 0.286935653 | 11.10942082 | 145.2921461 | 1.85392E−33 | 3.18835E−31 |
| Rplp1 | −0.248269602 | 11.7032567 | 144.8967265 | 2.26224E−33 | 3.84962E−31 |
| Slc15a3 | 0.801237735 | 9.227334772 | 144.1671838 | 3.26617E−33 | 5.50009E−31 |
| Stk24 | 0.558224742 | 9.608522084 | 144.136344 | 3.31727E−33 | 5.52856E−31 |
| Il1r2 | 1.188001348 | 9.256675468 | 142.1821627 | 8.87259E−33 | 1.46362E−30 |
| Ccr1 | 0.904615136 | 9.184947429 | 142.1482447 | 9.02541E−33 | 1.47379E−30 |
| Jund | 0.428678506 | 11.83414679 | 141.6222609 | 1.17619E−32 | 1.90143E−30 |
| Traf1 | 0.437567505 | 10.10521315 | 141.2746865 | 1.40113E−32 | 2.24264E−30 |
| Lilrb4a | 0.639236426 | 9.646913438 | 141.0375314 | 1.57883E−32 | 2.50229E−30 |
| Rpl41 | −0.169568893 | 12.79520696 | 140.2066552 | 2.39896E−32 | 3.7652E−30 |
| Rpl19 | −0.186575538 | 12.11746352 | 138.3592406 | 6.08169E−32 | 9.45352E−30 |
| Uhrf2 | 0.663533091 | 9.554448493 | 137.618841 | 8.82976E−32 | 1.35945E−29 |
| Nfkbiz | 0.589490901 | 9.633444859 | 137.3518839 | 1.01003E−31 | 1.54039E−29 |
| Gm8730 | −0.391879071 | 10.44464909 | 137.2040135 | 1.08811E−31 | 1.64397E−29 |
| Eif1 | 0.221160233 | 11.85893927 | 136.6927236 | 1.40766E−31 | 2.10706E−29 |
| Sdc4 | 0.864197912 | 9.184332418 | 136.104023 | 1.89346E−31 | 2.80823E−29 |
| Nrgn | 0.79593449 | 9.169490694 | 134.3836196 | 4.50361E−31 | 6.61867E−29 |
| Ikzf3 | 0.758912682 | 9.310513762 | 134.3025958 | 4.6912E−31 | 6.83225E−29 |
| Gpr65 | 0.721922552 | 9.365534671 | 133.4092603 | 7.35695E−31 | 1.0619E−28 |
| ler5l | 0.917735422 | 9.22331753 | 132.9741944 | 9.15947E−31 | 1.31037E−28 |
| Sla | 0.573929064 | 9.546171489 | 132.091611 | 1.42872E−30 | 2.02602E−28 |
| Ptms | 0.706758953 | 9.287837433 | 131.8631613 | 1.60296E−30 | 2.25335E−28 |
| Epsti1 | −0.531779216 | 9.946031824 | 129.0243258 | 6.69923E−30 | 9.33618E−28 |
| Samsn1 | 0.565617595 | 9.795395326 | 127.6319862 | 1.3511E−29 | 1.86683E−27 |
| Btg1 | 0.316118724 | 12.25005454 | 126.0369939 | 3.01804E−29 | 4.13472E−27 |
| Rpl4 | −0.200905454 | 11.68898179 | 125.9194397 | 3.20222E−29 | 4.35018E−27 |
| Rpl22l1 | −0.325682762 | 10.87574132 | 122.9210326 | 1.45111E−28 | 1.95489E−26 |
| Rps24 | −0.209984223 | 12.00584899 | 122.8780161 | 1.48291E−28 | 1.98122E−26 |
| Il2ra | 0.732900379 | 9.266099444 | 122.2199043 | 2.06619E−28 | 2.73787E−26 |
| Cd74 | 1.042070273 | 9.434236978 | 120.8762601 | 4.06726E−28 | 5.34563E−26 |
| Rabgap1l | 0.625903862 | 9.328937377 | 120.4547377 | 5.03015E−28 | 6.55785E−26 |
| Bcl211 | 0.671196005 | 9.376444233 | 119.2060708 | 9.43956E−28 | 1.21377E−25 |
| Rps27 | −0.20992776 | 13.23488676 | 119.2017178 | 9.4603E−28 | 1.21377E−25 |
| Fas | 0.720279276 | 9.354790431 | 119.0791946 | 1.00631E−27 | 1.28094E−25 |
| Rps23 | −0.172279837 | 12.48529882 | 117.8778644 | 1.844E−27 | 2.32892E−25 |
| Rpl17 | −0.175623878 | 12.29663229 | 116.7248327 | 3.29791E−27 | 4.13287E−25 |
| Rab11fip1 | 0.683102221 | 9.24534037 | 116.0444077 | 4.64772E−27 | 5.77962E−25 |
| Ctsb | 0.491115464 | 9.553686158 | 115.6909048 | 5.55461E−27 | 6.85464E−25 |
| Csf1 | 0.893917069 | 9.192117276 | 114.1779361 | 1.19125E−26 | 1.45892E−24 |
| Batf | 0.556500291 | 9.653651878 | 113.1282182 | 2.02263E−26 | 2.45848E−24 |
| Camk2n1 | 0.677140769 | 9.340531404 | 112.320738 | 3.0394E−26 | 3.66679E−24 |
| Lmnb1 | 0.496811846 | 9.781495667 | 112.1642896 | 3.28895E−26 | 3.93846E−24 |
| Prr7 | 0.506933212 | 9.824433563 | 112.1237154 | 3.35695E−26 | 3.99033E−24 |
| Wisp1 | 0.57314674 | 9.168186557 | 110.4669626 | 7.74251E−26 | 9.13616E−24 |
| Rps29 | −0.210381637 | 12.2446762 | 109.1261756 | 1.52277E−25 | 1.78385E−23 |
| Rps3a1 | −0.189505791 | 11.67389213 | 109.0426157 | 1.58834E−25 | 1.84727E−23 |
| Rpl12 | −0.280198685 | 10.99235633 | 108.860878 | 1.74086E−25 | 2.01019E−23 |
| Plk2 | 0.750446155 | 9.17838771 | 108.6038869 | 1.98186E−25 | 2.27225E−23 |
| Kdm2b | 0.56499929 | 9.446703124 | 108.5076284 | 2.08048E−25 | 2.36852E−23 |
| Wnk1 | 0.492798635 | 9.627957555 | 108.2227911 | 2.40202E−25 | 2.71545E−23 |
| Cd83 | 0.807532146 | 9.178407827 | 106.7863598 | 4.95842E−25 | 5.56651E−23 |
| Ifng | 0.886646176 | 9.686658841 | 106.6990667 | 5.18171E−25 | 5.77707E−23 |
| Rps27a | −0.184418544 | 11.80973167 | 106.5796788 | 5.50347E−25 | 6.09377E−23 |
| Cytip | 0.350491758 | 10.18840246 | 106.0027935 | 7.36313E−25 | 8.09744E−23 |
| Lilr4b | 0.511360258 | 9.778154625 | 105.7520711 | 8.35625E−25 | 9.12751E−23 |
| Nfkbib | 0.504241566 | 9.487223272 | 105.680851 | 8.66204E−25 | 9.39802E−23 |
| Scn1b | 0.509524106 | 9.169087199 | 105.5628385 | 9.19358E−25 | 9.90822E−23 |
| Rps15 | −0.203059364 | 11.4907216 | 102.7590505 | 3.78493E−24 | 4.05213E−22 |
| Il17a | 1.935391149 | 9.237168765 | 102.4199032 | 4.49166E−24 | 4.77712E−22 |
| Ucp2 | 0.363772503 | 10.01286384 | 101.8832566 | 5.88922E−24 | 6.22256E−22 |
| St6galnac3 | 0.675416095 | 9.261096607 | 101.3057309 | 7.88274E−24 | 8.27483E−22 |
| Rpl37a | −0.172845664 | 12.61561552 | 100.6238399 | 1.11221E−23 | 1.16E−21 |
| Cd6 | 0.418108973 | 9.931698154 | 100.1180091 | 1.43582E−23 | 1.48104E−21 |
| Rpl36 | −0.186839018 | 11.87411391 | 100.1145234 | 1.43835E−23 | 1.48104E−21 |
| Rpl9 | −0.172551122 | 11.84224161 | 99.8545999 | 1.64006E−23 | 1.67805E−21 |
| Rps13 | −0.169190789 | 11.9252029 | 97.97494346 | 4.2371E−23 | 4.30799E−21 |
| Ctla2a | −0.609825116 | 10.33555104 | 96.53343799 | 8.77493E−23 | 8.86597E−21 |
| Stat4 | 0.501673088 | 9.506547453 | 95.6224894 | 1.39018E−22 | 1.39588E−20 |
| Nr4a1 | 0.550612812 | 9.629989419 | 95.20303248 | 1.71827E−22 | 1.71466E−20 |
| Il7r | −0.558749116 | 9.75347996 | 95.17603619 | 1.74186E−22 | 1.72754E−20 |
| Rpl26 | −0.184107869 | 11.57423644 | 94.93673751 | 1.96568E−22 | 1.93763E−20 |
| Rps14 | −0.146333995 | 12.65498378 | 94.80316287 | 2.10289E−22 | 2.06033E−20 |
| Rasgrp2 | −0.687938938 | 9.37707238 | 94.64490756 | 2.27792E−22 | 2.21836E−20 |
| Dnajc15 | −0.415379881 | 9.893121495 | 94.12385202 | 2.96386E−22 | 2.86909E−20 |
| Gzmk | −1.043301711 | 9.363674736 | 94.00786796 | 3.14272E−22 | 3.02412E−20 |
| Rpl35a | −0.177783341 | 11.72342483 | 93.76286064 | 3.55684E−22 | 3.40236E−20 |
| Rpl37 | −0.16620817 | 12.07301201 | 93.58838916 | 3.8846E−22 | 3.69402E−20 |
| Emb | 0.407065297 | 10.25095358 | 93.57139524 | 3.91809E−22 | 3.70409E−20 |
| Gch1 | 0.570152772 | 9.459450391 | 93.03068278 | 5.14899E−22 | 4.83945E−20 |
| D16Ertd472e | 0.453585869 | 9.624017665 | 92.73130439 | 5.98986E−22 | 5.59723E−20 |
| Dgat1 | 0.530352135 | 9.77868262 | 92.53634073 | 6.60998E−22 | 6.1412E−20 |
| Nfkbia | 0.423596882 | 10.83366717 | 92.43061048 | 6.97272E−22 | 6.4412E−20 |
| Ly6c2 | −1.111488738 | 9.265996796 | 91.81855201 | 9.49997E−22 | 8.72593E−20 |
| Rpl3 | −0.158339089 | 12.09253162 | 91.51920698 | 1.10514E−21 | 1.00936E−19 |
| Zc3h12a | 0.484764166 | 9.536813666 | 90.15458744 | 2.2026E−21 | 2.00041E−19 |
| Rps9 | −0.162455931 | 11.99537323 | 90.08851031 | 2.2774E−21 | 2.05679E−19 |
| Tnfrsf1b | 0.516562473 | 9.452782192 | 89.6453515 | 2.84917E−21 | 2.55888E−19 |
| Rpl27a | −0.166874022 | 11.73983306 | 89.41629258 | 3.19891E−21 | 2.85711E−19 |
| Pdcd1 | 0.790619463 | 9.244434269 | 89.09231595 | 3.76812E−21 | 3.347E−19 |
| Myl12a | 0.247801136 | 10.67832871 | 89.02278242 | 3.90292E−21 | 3.44779E−19 |
| Pim3 | 0.642188756 | 9.328476745 | 88.91325482 | 4.1251E−21 | 3.62426E−19 |
| Rpl23 | −0.182988453 | 11.70859607 | 88.24623254 | 5.77932E−21 | 5.05019E−19 |
| Il22 | 1.587785436 | 9.48289887 | 88.01465782 | 6.49712E−21 | 5.64691E−19 |
| S100a11 | 0.291981912 | 10.60259181 | 87.37637479 | 8.97158E−21 | 7.75586E−19 |
| Tgif1 | 0.448665946 | 9.72028063 | 87.1158878 | 1.02345E−20 | 8.80059E−19 |
| Rpl39 | −0.181027763 | 11.77727203 | 86.79123751 | 1.20603E−20 | 1.03157E−18 |
| Sytl3 | 0.484538559 | 9.50441997 | 86.48786684 | 1.40597E−20 | 1.19626E−18 |
| Mir155hg | 0.675607589 | 9.203846162 | 86.09119012 | 1.71827E−20 | 1.45432E−18 |
| Hif1a | 0.401934609 | 9.774243864 | 86.00779175 | 1.79228E−20 | 1.50906E−18 |
| Itgb1 | −0.867290308 | 9.299264735 | 85.97505545 | 1.8222E−20 | 1.5263E−18 |
| Smim3 | 0.576452859 | 9.350052324 | 85.96468815 | 1.83178E−20 | 1.52642E−18 |
| Rpl6 | −0.160525203 | 11.76079317 | 84.93181841 | 3.08833E−20 | 2.56031E−18 |
| Rps20 | −0.237673584 | 11.06855654 | 84.75338603 | 3.38E−20 | 2.78781E−18 |
| Bcl2a1a | 0.64471644 | 9.252650209 | 84.52563174 | 3.79266E−20 | 3.11229E−18 |
| Ccl1 | 1.624101666 | 9.258098088 | 83.86340919 | 5.30168E−20 | 4.30688E−18 |
| Tmem176b | 0.721063124 | 9.448095178 | 83.87163544 | 5.27966E−20 | 4.30688E−18 |
| Cox17 | 0.352580634 | 9.818116404 | 83.64777925 | 5.91266E−20 | 4.77921E−18 |
| Rpl21 | −0.169864625 | 11.76872215 | 83.24580779 | 7.2459E−20 | 5.82772E−18 |
| Hmgb2 | −0.387568788 | 10.71130337 | 82.84554995 | 8.87218E−20 | 7.10038E−18 |
| Commd8 | −0.54296912 | 9.438213878 | 82.57656026 | 1.01655E−19 | 8.09538E−18 |
| Uap1 | 0.520815246 | 9.373356657 | 82.55889475 | 1.02568E−19 | 8.12801E−18 |
| Eef2 | −0.185290498 | 11.29631996 | 80.9150407 | 2.35633E−19 | 1.85817E−17 |
| Mmp9 | 0.58263451 | 9.175261184 | 80.89442021 | 2.38105E−19 | 1.86855E−17 |
| Emp3 | −0.437281554 | 9.864612689 | 80.86112424 | 2.42151E−19 | 1.89112E−17 |
| Crem | 0.520771771 | 10.06693577 | 80.83319269 | 2.45598E−19 | 1.90881E−17 |
| Tmem176a | 0.756251292 | 9.404489195 | 80.19433366 | 3.39339E−19 | 2.62476E−17 |
| Itm2c | 0.438982008 | 9.518096519 | 79.53250521 | 4.74357E−19 | 3.65164E−17 |
| Itk | 0.389293322 | 9.814325519 | 79.31793277 | 5.28776E−19 | 4.05127E−17 |
| Crlf2 | 0.486705303 | 9.388760459 | 79.07850293 | 5.96903E−19 | 4.55167E−17 |
| Ms4a6b | −0.359447432 | 10.19493409 | 78.10427151 | 9.77424E−19 | 7.41832E−17 |
| Arf6 | 0.343694888 | 9.853374067 | 78.01575022 | 1.02222E−18 | 7.72208E−17 |
| Eea1 | 0.570647144 | 9.220110798 | 77.7632572 | 1.16161E−18 | 8.73423E−17 |
| Tmem158 | 0.422938332 | 9.165141433 | 77.63465939 | 1.23975E−18 | 9.27864E−17 |
| Fth1 | 0.215979972 | 11.58471842 | 77.57335302 | 1.27884E−18 | 9.52705E−17 |
| Prkar1a | 0.274347844 | 10.10979937 | 76.99987246 | 1.70969E−18 | 1.26784E−16 |
| Rpl11 | −0.151171628 | 11.70903164 | 76.76522996 | 1.92538E−18 | 1.42126E−16 |
| Stat5a | 0.552298427 | 9.260743354 | 76.06349512 | 2.74689E−18 | 2.01846E−16 |
| Rpl23a | −0.147657414 | 12.17556349 | 75.49517478 | 3.66301E−18 | 2.67947E−16 |
| Cd28 | −0.347012261 | 10.37952986 | 75.2579058 | 4.13073E−18 | 3.00799E−16 |
| Rps2 | −0.18944846 | 11.63567309 | 74.93771616 | 4.858E−18 | 3.52173E−16 |
| Havcr2 | 0.539582937 | 9.180313486 | 73.34910354 | 1.08631E−17 | 7.83989E−16 |
| Nfkbid | 0.644113236 | 9.347296213 | 72.72063103 | 1.49364E−17 | 1.07317E−15 |
| Cbx3 | −0.581747509 | 9.518039516 | 72.62422047 | 1.56842E−17 | 1.12191E−15 |
| Rpl24 | −0.15447822 | 11.77225336 | 72.39600541 | 1.7607E−17 | 1.2539E−15 |
| Sh2d2a | 0.376644419 | 9.846354303 | 71.99999673 | 2.15198E−17 | 1.52583E−15 |
| Lef1 | −0.609615782 | 9.289425739 | 71.88108079 | 2.28565E−17 | 1.61353E−15 |
| Gm27166 | 0.407317455 | 9.163327075 | 71.56233274 | 2.68636E−17 | 1.88816E−15 |
| AW112010 | 0.282452628 | 11.38322545 | 71.2150772 | 3.20331E−17 | 2.24176E−15 |
| Serpina3g | 0.534098778 | 9.674243135 | 70.42584586 | 4.77897E−17 | 3.33003E−15 |
| Ifitm1 | −1.804896002 | 9.599666975 | 69.60975443 | 7.22786E−17 | 5.01483E−15 |
| Cish | 0.464190484 | 9.408302432 | 69.50815718 | 7.60991E−17 | 5.25734E−15 |
| Myl12b | 0.261240272 | 10.14192399 | 69.43717684 | 7.88876E−17 | 5.4268E−15 |
| Hccs | 0.494564115 | 9.354685384 | 69.31445298 | 8.39521E−17 | 5.75072E−15 |
| Ubald2 | 0.304205172 | 10.62012763 | 69.07611792 | 9.47356E−17 | 6.46201E−15 |
| Ppp1r14c | 0.355051825 | 9.162055719 | 68.72964003 | 1.12931E−16 | 7.67075E−15 |
| Ubl3 | 0.399042618 | 9.565981005 | 67.66623714 | 1.93651E−16 | 1.30986E−14 |
| Rplp2 | −0.157925517 | 11.91006486 | 67.41326155 | 2.20163E−16 | 1.48298E−14 |
| Rpl30 | −0.196263195 | 10.96407438 | 66.75449795 | 3.07515E−16 | 2.06277E−14 |
| Rpl36a | −0.20636157 | 11.04527747 | 65.99089756 | 4.5301E−16 | 3.02618E−14 |
| Ifitm3 | −0.741460054 | 9.717437467 | 65.84393111 | 4.88081E−16 | 3.24704E−14 |
| Pycard | −0.484732372 | 9.57708276 | 65.33967505 | 6.30393E−16 | 4.17661E−14 |
| Ptp4a2 | 0.290548225 | 9.957583537 | 65.07030211 | 7.22731E−16 | 4.76884E−14 |
| Ccl5 | −0.614177482 | 13.31111049 | 65.02719932 | 7.38714E−16 | 4.85449E−14 |
| H2-Q7 | 0.238928118 | 10.62098954 | 64.16806184 | 1.14246E−15 | 7.47732E−14 |
| Nfil3 | 0.543821195 | 9.279162762 | 64.01922369 | 1.23211E−15 | 8.03157E−14 |
| Gnb2l1 | −0.219333271 | 10.54007349 | 63.52463448 | 1.58374E−15 | 1.02822E−13 |
| Tmsb4x | 0.134395465 | 14.24920488 | 63.32329405 | 1.75418E−15 | 1.13432E−13 |
| Srxn1 | 0.443413556 | 9.179960772 | 62.81840721 | 2.2667E−15 | 1.4599E−13 |
| Dusp4 | 0.542015029 | 9.244687523 | 62.16159044 | 3.16399E−15 | 2.02973E−13 |
| Ifi27 | 0.364029871 | 9.579454659 | 61.73449754 | 3.93034E−15 | 2.51138E−13 |
| Dennd5a | 0.486880142 | 9.227017994 | 61.55510006 | 4.30525E−15 | 2.74011E−13 |
| Ube2l6 | 0.494919463 | 9.197355951 | 61.47362964 | 4.48713E−15 | 2.84466E−13 |
| Nrip1 | 0.453730601 | 9.582617594 | 61.30329582 | 4.89261E−15 | 3.0896E−13 |
| Rnf19b | 0.466509748 | 9.379266424 | 60.94088293 | 5.88147E−15 | 3.6996E−13 |
| Dad1 | 0.255140794 | 10.03535203 | 60.85328081 | 6.14909E−15 | 3.85296E−13 |
| Cd81 | 0.489308098 | 9.170580291 | 60.72356256 | 6.56793E−15 | 4.0995E−13 |
| Adora2a | 0.449099604 | 9.426691213 | 60.60452645 | 6.97734E−15 | 4.33829E−13 |
| Glipr2 | −0.475835778 | 9.484107632 | 60.27095878 | 8.2658E−15 | 5.11973E−13 |
| 2810417H13Rik | −1.104938967 | 9.337315681 | 60.12562098 | 8.89924E−15 | 5.49103E−13 |
| Rpl28 | −0.211074368 | 10.72552157 | 59.95350725 | 9.71248E−15 | 5.97003E−13 |
| Cxcr3 | −0.481054249 | 9.551371405 | 59.90276731 | 9.96612E−15 | 6.10274E−13 |
| Hk2 | 0.558083103 | 9.291512241 | 59.86859259 | 1.01407E−14 | 6.1862E−13 |
| Ckb | 0.537418423 | 9.335711504 | 59.82713032 | 1.03566E−14 | 6.29415E−13 |
| Pqlc1 | 0.536550639 | 9.21423837 | 59.77158922 | 1.0653E−14 | 6.45006E−13 |
| Ubl5 | 0.227393519 | 10.27393085 | 59.43006853 | 1.26718E−14 | 7.64375E−13 |
| Hlf | 0.469359308 | 9.183131189 | 59.07360438 | 1.51883E−14 | 9.12766E−13 |
| Ggt1 | −0.482757461 | 9.203324774 | 58.96486419 | 1.60513E−14 | 9.61055E−13 |
| Eef1g | −0.232791111 | 10.38514948 | 58.86655143 | 1.68736E−14 | 1.00657E−12 |
| Tff1 | 0.800550053 | 9.186702666 | 58.58119672 | 1.95072E−14 | 1.15939E−12 |
| Asb2 | 0.474910444 | 9.480773625 | 58.26906703 | 2.28611E−14 | 1.35374E−12 |
| Rpl15 | −0.196567365 | 10.73583328 | 58.16857192 | 2.40592E−14 | 1.41949E−12 |
| Pdcd4 | −0.433321184 | 9.736958579 | 57.54915517 | 3.29637E−14 | 1.93779E−12 |
| Gm26740 | 0.385787547 | 9.658978649 | 57.29699619 | 3.74727E−14 | 2.19487E−12 |
| Arf4 | 0.289627395 | 9.97479051 | 56.75239941 | 4.94287E−14 | 2.88471E−12 |
| Kit | 0.361389295 | 9.167862474 | 56.47666634 | 5.68692E−14 | 3.30701E−12 |
| Stmn1 | −0.996361487 | 9.43019399 | 56.43186032 | 5.818E−14 | 3.3711E−12 |
| Btg2 | 0.279901817 | 10.53791954 | 56.13951487 | 6.75066E−14 | 3.89754E−12 |
| Esm1 | −0.770430889 | 9.307911702 | 56.02164157 | 7.16777E−14 | 4.12363E−12 |
| Jun | −0.827066689 | 9.603760111 | 55.34047173 | 1.0136E−13 | 5.81059E−12 |
| Rps8 | −0.149468965 | 11.59257976 | 55.20418787 | 1.08637E−13 | 6.20575E−12 |
| Rps27rt | −0.255171721 | 11.16195099 | 55.0140882 | 1.19669E−13 | 6.81186E−12 |
| Cd44 | 0.362141582 | 9.560095995 | 54.61856376 | 1.46347E−13 | 8.30119E−12 |
| Myo1g | 0.396341726 | 9.439415321 | 54.50805268 | 1.54812E−13 | 8.75068E−12 |
| Calr | 0.281098495 | 10.02078334 | 54.09506061 | 1.91021E−13 | 1.07597E−11 |
| Crbn | 0.353059697 | 9.708183401 | 53.96723433 | 2.03861E−13 | 1.14431E−11 |
| Cd3e | 0.233574904 | 10.3749064 | 53.67655956 | 2.36367E−13 | 1.32218E−11 |
| Nr3c1 | 0.435805045 | 9.448742099 | 53.64404633 | 2.40311E−13 | 1.33961E−11 |
| Sp100 | −0.41585345 | 9.553686189 | 53.59274678 | 2.46668E−13 | 1.36563E−11 |
| Klhl6 | −0.508204735 | 9.316905875 | 53.59681118 | 2.46159E−13 | 1.36563E−11 |
| Rps12 | −0.210582225 | 10.77061599 | 53.54559432 | 2.5266E−13 | 1.39403E−11 |
| S1pr4 | −0.454655152 | 9.466008758 | 52.71694568 | 3.85253E−13 | 2.11837E−11 |
| Aplp1 | 0.432674667 | 9.212555161 | 52.59179415 | 4.10601E−13 | 2.25009E−11 |
| Gimap4 | −0.240367235 | 10.52502448 | 52.52162603 | 4.25537E−13 | 2.32406E−11 |
| Plk3 | 0.478782538 | 9.376309307 | 52.27013392 | 4.83675E−13 | 2.63269E−11 |
| Nfkb2 | 0.369018011 | 9.521818169 | 52.2111144 | 4.98432E−13 | 2.70391E−11 |
| Herpud1 | 0.372438068 | 9.555345247 | 52.01800557 | 5.4994E−13 | 2.97336E−11 |
| Cotl1 | −0.247357975 | 10.46113625 | 51.80498811 | 6.12959E−13 | 3.30303E−11 |
| Rpl27 | −0.143507587 | 11.47263607 | 51.4272134 | 7.43014E−13 | 3.99055E−11 |
| Cdkn1b | −0.502025248 | 9.417453754 | 51.41112286 | 7.49129E−13 | 4.01007E−11 |
| Slc25a3 | 0.260721523 | 9.949542602 | 51.20807313 | 8.30763E−13 | 4.43238E−11 |
| 1500009L16Rik | −0.542177966 | 9.246966845 | 51.17448647 | 8.45099E−13 | 4.49404E−11 |
| Tbc1d10c | −0.420702224 | 9.522664674 | 51.06091305 | 8.95437E−13 | 4.74611E−11 |
| Zap70 | 0.337335162 | 9.555404972 | 50.97047958 | 9.37656E−13 | 4.94794E−11 |
| Csf2 | 0.79245829 | 9.183961698 | 50.96633727 | 9.39637E−13 | 4.94794E−11 |
| Nabp1 | 0.36892163 | 9.632417698 | 50.79434236 | 1.02569E−12 | 5.38354E−11 |
| Serpinb1a | 0.721125754 | 9.512639892 | 50.70625213 | 1.07277E−12 | 5.61244E−11 |
| Rpl34 | −0.118076419 | 11.98010888 | 50.63819169 | 1.11063E−12 | 5.79174E−11 |
| Rpl7a | −0.185997252 | 10.71264639 | 50.5255593 | 1.17623E−12 | 6.11412E−11 |
| Atp1b3 | −0.43409369 | 9.41300625 | 50.42513044 | 1.23799E−12 | 6.41452E−11 |
| Ramp3 | 0.557408857 | 9.936798851 | 50.34255136 | 1.29119E−12 | 6.66882E−11 |
| Tmbim6 | 0.218312849 | 10.08427218 | 49.92911906 | 1.59401E−12 | 8.20663E−11 |
| Csnk1a1 | 0.284142186 | 9.708202059 | 49.80980859 | 1.69395E−12 | 8.69346E−11 |
| Rpl38 | −0.14321935 | 11.67454895 | 49.67673704 | 1.81283E−12 | 9.2741E−11 |
| Ubb | 0.178674236 | 12.9047306 | 49.33933111 | 2.15302E−12 | 1.09797E−10 |
| Itgav | 0.460676285 | 9.245494327 | 49.23555497 | 2.26999E−12 | 1.15398E−10 |
| Dnajb1 | −0.621384827 | 10.31779183 | 49.17154489 | 2.34528E−12 | 1.18852E−10 |
| Slamf6 | −0.548414682 | 9.319043642 | 49.08519847 | 2.45083E−12 | 1.23813E−10 |
| Tmem123 | 0.308551986 | 9.654226375 | 48.55589077 | 3.21009E−12 | 1.61664E−10 |
| Cxcr4 | 0.458227755 | 9.20350922 | 47.73294408 | 4.88415E−12 | 2.45209E−10 |
| Vamp8 | 0.288954516 | 9.668612941 | 47.46653284 | 5.59511E−12 | 2.79168E−10 |
| Psme2 | 0.231125589 | 10.07701741 | 47.47010135 | 5.58493E−12 | 2.79168E−10 |
| Dusp5 | 0.282745296 | 9.994462064 | 47.30156077 | 6.08637E−12 | 3.02746E−10 |
| Tubb6 | 0.311404816 | 9.16596651 | 47.00909462 | 7.06581E−12 | 3.50386E−10 |
| Xbp1 | 0.346296453 | 9.544395327 | 46.81439795 | 7.8038E−12 | 3.84513E−10 |
| Gabpb1 | 0.407342294 | 9.478019755 | 46.80899053 | 7.82536E−12 | 3.84513E−10 |
| Gimap6 | −0.250117271 | 10.46581517 | 46.81130486 | 7.81612E−12 | 3.84513E−10 |
| Tcf7 | −0.458371226 | 9.419705743 | 46.79725798 | 7.87235E−12 | 3.8565E−10 |
| Il21 | 0.334899575 | 9.167189925 | 46.65641679 | 8.45895E−12 | 4.13134E−10 |
| Ppp1r11 | 0.315315515 | 9.56745842 | 46.63219242 | 8.56417E−12 | 4.17013E−10 |
| Serpina3f | 0.611380344 | 9.259717975 | 46.43170786 | 9.48679E−12 | 4.60551E−10 |
| C2cd4b | 0.305709635 | 9.163047524 | 45.77620863 | 1.32564E−11 | 6.41625E−10 |
| Ccnd2 | 0.321927763 | 9.824613466 | 45.72543169 | 1.36045E−11 | 6.56508E−10 |
| Pdlim1 | −0.473620534 | 9.286476238 | 45.16874379 | 1.80767E−11 | 8.69727E−10 |
| Ttc39c | 0.403933345 | 9.318650364 | 44.88156534 | 2.09319E−11 | 1.00411E−09 |
| Ciapin1 | 0.392937977 | 9.338637993 | 44.67693901 | 2.32378E−11 | 1.11143E−09 |
| Smox | 0.466037737 | 9.201962775 | 44.63380471 | 2.37554E−11 | 1.13283E−09 |
| Itpr1 | 0.395519874 | 9.29809745 | 44.54267134 | 2.48873E−11 | 1.18332E−09 |
| Csrnp1 | 0.398371772 | 9.467469906 | 44.4996176 | 2.54407E−11 | 1.20608E−09 |
| Eef1b2 | −0.234877371 | 10.07539868 | 44.48805958 | 2.55913E−11 | 1.20968E−09 |
| Coro1a | −0.210386427 | 10.77304014 | 44.28150328 | 2.84391E−11 | 1.34037E−09 |
| Ly6g5b | 0.453449596 | 9.223291205 | 44.27562159 | 2.85246E−11 | 1.34049E−09 |
| Hmgn2 | −0.516427634 | 9.445227782 | 44.18024862 | 2.99488E−11 | 1.40334E−09 |
| Arpc3 | 0.193568182 | 10.54007412 | 44.16091507 | 3.02461E−11 | 1.41317E−09 |
| Itgb8 | 0.150825817 | 9.157971212 | 44.02687611 | 3.23898E−11 | 1.50897E−09 |
| Rasa3 | −0.456532606 | 9.237633903 | 43.88549931 | 3.48159E−11 | 1.61734E−09 |
| 4833407H14Rik | 0.432971571 | 9.210586484 | 43.61892105 | 3.98963E−11 | 1.84803E−09 |
| Srsf5 | 0.224610507 | 10.09105297 | 42.90253888 | 5.75359E−11 | 2.64993E−09 |
| Tnfrsf26 | −0.421012345 | 9.206974678 | 42.90779839 | 5.73815E−11 | 2.64993E−09 |
| Uba52 | −0.238263321 | 10.23996246 | 42.68346594 | 6.43539E−11 | 2.95553E−09 |
| Tg | 0.21283359 | 9.163083285 | 42.49207931 | 7.0969E−11 | 3.2501E−09 |
| Eef1a1 | −0.107002554 | 12.38177109 | 42.46218858 | 7.20619E−11 | 3.29083E−09 |
| 2700094K13Rik | −0.438643961 | 9.385257191 | 42.44288494 | 7.27767E−11 | 3.31411E−09 |
| Cdca3 | −0.392634098 | 9.196718956 | 42.4258423 | 7.34136E−11 | 3.33372E−09 |
| Ptpn 18 | −0.226744978 | 10.2346755 | 42.2886025 | 7.87502E−11 | 3.56604E−09 |
| Il10 | 0.594261639 | 9.174516082 | 42.24369134 | 8.05795E−11 | 3.63868E−09 |
| Rheb | 0.295652408 | 9.556041475 | 42.10431461 | 8.65321E−11 | 3.89659E−09 |
| Ebi3 | 0.367076397 | 9.178699077 | 41.85523589 | 9.82872E−11 | 4.41364E−09 |
| Birc5 | −0.679292998 | 9.251484435 | 41.7880138 | 1.01725E−10 | 4.55537E−09 |
| Traf4 | 0.397393716 | 9.359798204 | 41.65359192 | 1.08965E−10 | 4.85815E−09 |
| Naca | −0.15683386 | 10.80133383 | 41.65139444 | 1.09088E−10 | 4.85815E−09 |
| Xcl1 | 0.951964559 | 9.405054453 | 41.31879274 | 1.2932E−10 | 5.74335E−09 |
| Pdia6 | 0.340678053 | 9.493316871 | 41.26199076 | 1.33133E−10 | 5.8965E−09 |
| Mki67 | −0.402680653 | 9.20298528 | 41.2101277 | 1.36712E−10 | 6.0385E−09 |
| Cks1b | −0.56316805 | 9.261610399 | 41.09631286 | 1.44909E−10 | 6.38312E−09 |
| Klrb1b | 0.402897716 | 9.168673107 | 41.05896621 | 1.47705E−10 | 6.48858E−09 |
| Cd4 | 0.28495697 | 9.643227058 | 40.87227781 | 1.6251E−10 | 7.11962E−09 |
| Maf | 0.361126024 | 9.568694911 | 40.76659514 | 1.7154E−10 | 7.49493E−09 |
| Ube2c | −0.603273399 | 9.233354826 | 40.64117095 | 1.82912E−10 | 7.97022E−09 |
| Ifrd1 | 0.287187244 | 10.02592468 | 40.57737019 | 1.88983E−10 | 8.21262E−09 |
| Rpl31 | −0.16139966 | 10.87152232 | 40.55059192 | 1.9159E−10 | 8.30362E−09 |
| Dusp10 | −0.470357283 | 9.475528346 | 40.43935976 | 2.02814E−10 | 8.74317E−09 |
| Rbm3 | −0.193760967 | 10.73099358 | 40.43969521 | 2.02779E−10 | 8.74317E−09 |
| Tacc3 | −0.331766486 | 9.190029393 | 40.29368335 | 2.18514E−10 | 9.39495E−09 |

TABLE 3G
LPL-6 vs. All

| GeneSymbol | logFC | logCPM | LR | PValue | FDR |
|---|---|---|---|---|---|
| Ccl5 | −2.119299251 | 13.31114939 | 553.6615868 | 2.0112E−122 | 3.2513E−118 |
| Actb | −1.037279996 | 13.40503113 | 371.7898077 | 7.62957E−83 | 6.16698E−79 |
| Malat1 | −0.585159603 | 14.66861581 | 331.2421667 | 5.15517E−74 | 2.77795E−70 |
| Tmsb4x | 0.298469035 | 14.2491977 | 309.5886424 | 2.68433E−69 | 1.08487E−65 |
| Jund | −0.708702846 | 11.83423061 | 291.9011257 | 1.91568E−65 | 6.19377E−62 |
| Kdm6b | −1.224505023 | 9.740630281 | 265.2542981 | 1.22814E−59 | 3.30902E−56 |
| Pim1 | −0.747674341 | 10.82590639 | 254.0062819 | 3.47585E−57 | 8.02723E−54 |
| Klf6 | −0.974714591 | 9.897242743 | 244.207249 | 4.7574E−55 | 9.61352E−52 |
| Icos | 0.600295791 | 10.25640856 | 233.1244444 | 1.24157E−52 | 2.23014E−49 |
| Dnajb1 | −1.387544483 | 10.31787822 | 204.9269834 | 1.75678E−46 | 2.84001E−43 |
| Tnfrsf4 | 0.656744874 | 10.62223191 | 193.5807006 | 5.25749E−44 | 7.7266E−41 |
| Jun | −1.611103318 | 9.603803257 | 192.9520131 | 7.211E−44 | 9.71443E−41 |
| Klf2 | −1.352858036 | 9.960820117 | 190.3008952 | 2.73308E−43 | 3.39869E−40 |
| Junb | −0.53873041 | 11.89128335 | 178.4624427 | 1.04988E−40 | 1.21231E−37 |
| Klrk1 | −1.134540447 | 9.564496772 | 163.4242247 | 2.0209E−37 | 2.17799E−34 |
| Klrc1 | −1.002669535 | 9.658443855 | 158.9563459 | 1.91284E−36 | 1.93269E−33 |
| Rps11 | 0.194712205 | 12.26304473 | 156.1743432 | 7.75479E−36 | 7.37435E−33 |
| Fos | −1.298192946 | 9.517399295 | 144.8988051 | 2.25988E−33 | 2.02962E−30 |
| Actg1 | 0.308629396 | 12.09922083 | 140.7810921 | 1.79643E−32 | 1.52848E−29 |
| Psme2 | 0.36279445 | 10.07701332 | 140.1516077 | 2.46638E−32 | 1.99358E−29 |
| Cd52 | 0.319567834 | 11.79358582 | 139.3304579 | 3.72932E−32 | 2.87087E−29 |
| Ifi27l2a | 0.787112867 | 9.817057964 | 136.8601041 | 1.29387E−31 | 9.50759E−29 |
| Cbx3 | −0.769736489 | 9.518048716 | 136.1258701 | 1.87275E−31 | 1.3163E−28 |
| Satb1 | 0.570904875 | 9.757032638 | 135.046907 | 3.2246E−31 | 2.17204E−28 |
| Nr4a1 | −0.731187746 | 9.630004042 | 133.3325151 | 7.64691E−31 | 4.9448E−28 |
| Nkg7 | −0.477227983 | 11.21025218 | 127.7495144 | 1.27342E−29 | 7.73982E−27 |
| Arpc2 | 0.244788106 | 10.79491082 | 127.7197104 | 1.29268E−29 | 7.73982E−27 |
| Tmsb10 | 0.208835701 | 12.94245518 | 126.3656535 | 2.55741E−29 | 1.47654E−26 |
| Rac2 | 0.253915556 | 11.09605368 | 126.0748863 | 2.96096E−29 | 1.65058E−26 |
| Dusp1 | −0.734404833 | 9.939529983 | 121.3508566 | 3.20189E−28 | 1.72539E−25 |
| Sh3bgrl3 | 0.259050953 | 11.24480074 | 121.1440221 | 3.55374E−28 | 1.85322E−25 |
| Pdcd4 | −0.608087104 | 9.736997086 | 120.6294586 | 4.60607E−28 | 2.32693E−25 |
| Ccl4 | −2.269830956 | 9.567609898 | 120.086778 | 6.0553E−28 | 2.96636E−25 |
| Tra2b | −0.476194641 | 9.814927917 | 118.9336673 | 1.08291E−27 | 5.14892E−25 |
| Fosb | −0.934865905 | 9.410873748 | 117.0575887 | 2.78852E−27 | 1.28798E−24 |
| Tubb5 | −0.629055114 | 10.0030371 | 114.7348946 | 8.9954E−27 | 4.03943E−24 |
| Eef2 | 0.193903782 | 11.29629909 | 111.5670215 | 4.44521E−26 | 1.9422E−23 |
| Rpl29 | 0.210961309 | 11.21424374 | 111.1928464 | 5.36859E−26 | 2.28391E−23 |
| Coro1a | 0.300729816 | 10.77301274 | 109.4044536 | 1.32332E−25 | 5.48533E−23 |
| Zfp36 | −0.929098819 | 9.294666471 | 107.2992323 | 3.82783E−25 | 1.54702E−22 |
| Cotl1 | 0.313916071 | 10.46110054 | 105.4791704 | 9.59007E−25 | 3.7813E−22 |
| Btg1 | −0.31432151 | 12.25010724 | 103.5727856 | 2.51001E−24 | 9.66116E−22 |
| Rplp0 | 0.166102954 | 12.38774335 | 103.4192727 | 2.71223E−24 | 1.01967E−21 |
| Crip1 | −0.502107257 | 11.66661525 | 103.2854441 | 2.90177E−24 | 1.06614E−21 |
| Cox4i1 | 0.21654735 | 10.68632092 | 101.9225287 | 5.77362E−24 | 2.07414E−21 |
| Ptpn22 | −0.418069094 | 10.09356864 | 101.6750761 | 6.54183E−24 | 2.29903E−21 |
| Myl6 | 0.224771123 | 10.93118441 | 98.12750402 | 3.92292E−23 | 1.34932E−20 |
| Tuba1b | −0.669934532 | 9.729931155 | 95.76193385 | 1.29563E−22 | 4.36358E−20 |
| Rgcc | −0.874994054 | 9.563667858 | 94.81868148 | 2.08647E−22 | 6.88366E−20 |
| Cdkn1b | −0.635174622 | 9.417482747 | 93.7734478 | 3.53786E−22 | 1.14386E−19 |
| Neat1 | −0.596555269 | 9.477111072 | 92.32207019 | 7.36583E−22 | 2.33482E−19 |
| Csrnp1 | −0.610932033 | 9.467484395 | 91.63024448 | 1.04484E−21 | 3.24824E−19 |
| Naca | 0.203875855 | 10.80131195 | 90.9215054 | 1.49486E−21 | 4.55961E−19 |
| Gem | −0.865598263 | 9.368397194 | 89.87230954 | 2.54038E−21 | 7.46686E−19 |
| Abhd2 | −0.664957436 | 9.292318122 | 89.87963305 | 2.53099E−21 | 7.46686E−19 |
| Rpl41 | 0.127967691 | 12.79519793 | 89.67974193 | 2.80008E−21 | 8.08322E−19 |
| Pfn1 | 0.239223472 | 12.14557892 | 89.50160675 | 3.0639E−21 | 8.68964E−19 |
| Btf3 | 0.194596928 | 10.71545921 | 88.29955513 | 5.62561E−21 | 1.56799E−18 |
| Ier2 | −0.464679761 | 9.828717858 | 88.24976501 | 5.76901E−21 | 1.58071E−18 |
| Rpgrip1 | −0.620409035 | 9.229072115 | 87.48124452 | 8.50828E−21 | 2.29241E−18 |
| Hspa1a | −1.404194987 | 11.00156878 | 87.25336404 | 9.54728E−21 | 2.53018E−18 |
| Rpl32 | 0.144936887 | 12.44733618 | 86.78944154 | 1.20712E−20 | 3.14747E−18 |
| Epsti1 | 0.367584515 | 9.946036789 | 86.67117338 | 1.28151E−20 | 3.2884E−18 |
| Rpl7a | 0.213884318 | 10.71261113 | 86.19936713 | 1.6268E−20 | 4.10919E−18 |
| Atp5e | 0.230435333 | 10.27341219 | 86.16667568 | 1.65391E−20 | 4.11341E−18 |
| Clta | 0.31374906 | 9.873288523 | 85.62610594 | 2.17386E−20 | 5.32465E−18 |
| Klrd1 | −0.780902756 | 9.372668746 | 85.1636242 | 2.74669E−20 | 6.6273E−18 |
| Ppp1ca | 0.265838001 | 10.29794445 | 85.06970008 | 2.88031E−20 | 6.84751E−18 |
| Rps27 | −0.178481761 | 13.23489548 | 83.42957086 | 6.60269E−20 | 1.54694E−17 |
| Eomes | −0.782994701 | 9.215525466 | 83.32009501 | 6.97866E−20 | 1.61167E−17 |
| Btg2 | −0.361177255 | 10.53796877 | 82.29599985 | 1.17159E−19 | 2.66759E−17 |
| Arhgdib | 0.246995265 | 10.73176252 | 81.72143316 | 1.56685E−19 | 3.51801E−17 |
| Srsf2 | −0.38575707 | 9.857793962 | 81.57579594 | 1.68667E−19 | 3.73517E−17 |
| Ifng | −0.925453687 | 9.686670275 | 81.19858443 | 2.04138E−19 | 4.45959E−17 |
| Rpl18 | 0.147120477 | 11.80688286 | 80.88213076 | 2.3959E−19 | 5.16429E−17 |
| B2m | 0.127460393 | 12.79981702 | 80.45008985 | 2.98141E−19 | 6.34177E−17 |
| Rps6 | 0.132843484 | 12.20837322 | 79.63837783 | 4.49607E−19 | 9.43941E−17 |
| Gzmk | −0.915585703 | 9.363683032 | 78.08817128 | 9.85423E−19 | 2.04235E−16 |
| Tsc22d3 | −0.554100907 | 9.704520503 | 77.67486703 | 1.21477E−18 | 2.48582E−16 |
| Atp5l | 0.208815411 | 10.47764488 | 76.5962148 | 2.09741E−18 | 4.23835E−16 |
| Klrc2 | −0.638997942 | 9.283451384 | 75.72542726 | 3.25983E−18 | 6.42663E−16 |
| Atp5h | 0.204975402 | 10.35716243 | 75.73020212 | 3.25195E−18 | 6.42663E−16 |
| Myl12a | 0.216154026 | 10.67829946 | 74.71071179 | 5.44996E−18 | 1.06149E−15 |
| Cldnd1 | 0.455613978 | 9.794877348 | 74.25249729 | 6.87378E−18 | 1.32288E−15 |
| Rpl24 | 0.143523244 | 11.77224479 | 74.13572633 | 7.29265E−18 | 1.38698E−15 |
| Tnfaip3 | −0.365730976 | 10.12511135 | 73.48927117 | 1.01185E−17 | 1.90204E−15 |
| Igtp | 0.489020478 | 9.509011969 | 72.98604439 | 1.3057E−17 | 2.4262E−15 |
| Zeb2 | −0.683047486 | 9.266621361 | 72.37282587 | 1.7815E−17 | 3.2727E−15 |
| Hspa1b | −0.982648588 | 9.33065317 | 71.96673669 | 2.18856E−17 | 3.9753E−15 |
| Gbp2 | 0.578294327 | 9.365760865 | 71.84703864 | 2.32542E−17 | 4.17698E−15 |
| Limd2 | 0.306608864 | 10.08910343 | 70.73863745 | 4.07827E−17 | 7.24497E−15 |
| Nr4a2 | −0.594424353 | 9.521309096 | 70.3636035 | 4.93216E−17 | 8.66666E−15 |
| Podnl1 | 0.474592382 | 9.399899052 | 69.83512388 | 6.44745E−17 | 1.12075E−14 |
| Ubb | −0.227426973 | 12.90474075 | 69.5319117 | 7.51881E−17 | 1.29308E−14 |
| Ctla4 | 0.371383943 | 10.15302447 | 68.7764088 | 1.10284E−16 | 1.87669E−14 |
| Ier5 | −0.446282214 | 9.710419 | 67.34884443 | 2.27475E−16 | 3.83059E−14 |
| Cyba | 0.228161069 | 10.43090095 | 67.27316027 | 2.36378E−16 | 3.93946E−14 |
| Ly6a | 0.344276567 | 10.37489399 | 66.83157909 | 2.95723E−16 | 4.87822E−14 |
| Rpsa | 0.151295131 | 11.60006986 | 65.95050999 | 4.62389E−16 | 7.55048E−14 |
| Izumo1r | 0.61967677 | 9.250974438 | 65.90232049 | 4.73833E−16 | 7.65999E−14 |
| Ptpn18 | 0.246625147 | 10.23468686 | 65.53911194 | 5.6972E−16 | 9.11891E−14 |
| Eef1a1 | 0.126330716 | 12.38174726 | 65.51205207 | 5.77597E−16 | 9.15434E−14 |
| Cd47 | 0.243733981 | 10.23573149 | 65.08241585 | 7.18302E−16 | 1.12739E−13 |
| Tnfrsf18 | 0.317256822 | 10.06771506 | 64.95225433 | 7.6735E−16 | 1.19279E−13 |
| Rpl28 | 0.196367442 | 10.72547914 | 64.85165546 | 8.07542E−16 | 1.24331E−13 |
| Dnaja1 | −0.382910023 | 10.76375183 | 64.28950963 | 1.07416E−15 | 1.6382E−13 |
| Cdkn1a | −0.572722919 | 9.693191434 | 64.04001936 | 1.21917E−15 | 1.84198E−13 |
| Pmaip1 | −0.551162792 | 9.419398949 | 63.93022725 | 1.28905E−15 | 1.92951E−13 |
| Psmb10 | 0.346016435 | 9.566302759 | 63.15714108 | 1.90856E−15 | 2.83063E−13 |
| Camk2n1 | −0.548998521 | 9.340541632 | 62.825393 | 2.25868E−15 | 3.31944E−13 |
| Rpa2 | −0.513404915 | 9.392689651 | 62.62460404 | 2.50109E−15 | 3.64258E−13 |
| Rbm39 | −0.201350849 | 10.68052293 | 62.46412644 | 2.71342E−15 | 3.91654E−13 |
| Rgs16 | −0.549477284 | 9.19690865 | 62.21207359 | 3.08391E−15 | 4.41191E−13 |
| Rpl7 | 0.144293297 | 11.21184535 | 62.08606475 | 3.2877E−15 | 4.66219E−13 |
| Srgn | 0.197435701 | 11.37082448 | 61.91810443 | 3.58043E−15 | 5.03314E−13 |
| Gapdh | 0.22553958 | 10.56198637 | 61.58565581 | 4.23896E−15 | 5.9075E−13 |
| Rps2 | 0.160373372 | 11.63563438 | 61.3663959 | 4.73829E−15 | 6.54694E−13 |
| Rgs2 | −0.521144086 | 10.0598516 | 60.74892465 | 6.48385E−15 | 8.88288E−13 |
| Atp5c1 | 0.250321016 | 9.90168537 | 60.61894058 | 6.92643E−15 | 9.40947E−13 |
| Gm26532 | −0.572078015 | 9.298938254 | 60.31224518 | 8.09423E−15 | 1.09043E−12 |
| Ctss | 0.407612199 | 9.35788439 | 59.39882781 | 1.28746E−14 | 1.72009E−12 |
| Rpl36al | 0.178394642 | 10.49579188 | 59.19840331 | 1.42549E−14 | 1.8889E−12 |
| Psma5 | 0.353113416 | 9.439553675 | 58.52395081 | 2.00831E−14 | 2.63954E−12 |
| Rpl3 | 0.117312584 | 12.09252558 | 58.41988325 | 2.1174E−14 | 2.75249E−12 |
| Gimap4 | 0.227922697 | 10.52504419 | 58.40977973 | 2.1283E−14 | 2.75249E−12 |
| Ppp1r16b | −0.456851556 | 9.488158562 | 58.18107506 | 2.39068E−14 | 3.06728E−12 |
| Gbp3 | 0.466119067 | 9.305848055 | 57.61787477 | 3.1832E−14 | 4.05193E−12 |
| Kmt2d | −0.509132586 | 9.2641734 | 57.50848455 | 3.36524E−14 | 4.21725E−12 |
| Srrm2 | −0.320172857 | 9.808268375 | 57.51207828 | 3.3591E−14 | 4.21725E−12 |
| Rora | −0.362384089 | 9.982422283 | 57.46868504 | 3.43403E−14 | 4.27035E−12 |
| Plek | −0.517944688 | 9.215823682 | 57.14493073 | 4.0485E−14 | 4.96758E−12 |
| Nfkbia | −0.369646528 | 10.83370997 | 57.14120793 | 4.05617E−14 | 4.96758E−12 |
| Myeov2 | 0.282121028 | 9.682137922 | 57.06950421 | 4.20679E−14 | 5.1133E−12 |
| Litaf | −0.519571904 | 9.445070319 | 56.35236309 | 6.05804E−14 | 7.30853E−12 |
| Cfl1 | 0.175243041 | 11.11282374 | 56.04269339 | 7.09143E−14 | 8.49185E−12 |
| Lmna | −0.624473963 | 9.713563886 | 55.98834182 | 7.2902E−14 | 8.66569E−12 |
| Rpl31 | 0.170535302 | 10.87149283 | 55.72060327 | 8.35382E−14 | 9.8575E−12 |
| Eif4a2 | −0.342532769 | 9.723875751 | 55.39883565 | 9.83947E−14 | 1.15264E−11 |
| Klf4 | −0.641217216 | 9.240094172 | 54.83835195 | 1.30862E−13 | 1.52195E−11 |
| Il22 | −1.523067898 | 9.482900463 | 54.56924671 | 1.50066E−13 | 1.73283E−11 |
| H3f3b | −0.169982377 | 12.34437077 | 54.47679797 | 1.57294E−13 | 1.80342E−11 |
| Psma2 | 0.235537521 | 9.873200775 | 54.25508068 | 1.76081E−13 | 2.0046E−11 |
| Ubl5 | 0.20032877 | 10.27392635 | 53.90086965 | 2.10864E−13 | 2.3838E−11 |
| Riok1 | 0.43384703 | 9.379913783 | 53.22660276 | 2.97206E−13 | 3.33656E−11 |
| Odc1 | 0.372271092 | 10.4535154 | 53.12397078 | 3.13148E−13 | 3.49127E−11 |
| Asb2 | 0.435113714 | 9.4807802 | 52.57892819 | 4.133E−13 | 4.54517E−11 |
| Tomm20 | 0.208801999 | 10.02363568 | 52.582178 | 4.12617E−13 | 4.54517E−11 |
| Sdf4 | 0.266427534 | 9.917368178 | 52.46598598 | 4.37765E−13 | 4.7817E−11 |
| Glrx | 0.371027895 | 9.565807787 | 52.40309399 | 4.52011E−13 | 4.87233E−11 |
| Ptma | −0.20232451 | 11.28818093 | 52.40274773 | 4.52091E−13 | 4.87233E−11 |
| Rps3 | 0.104436824 | 11.96704423 | 51.85522186 | 5.97476E−13 | 6.39655E−11 |
| Pkp3 | 0.335067835 | 9.709713743 | 51.70638646 | 6.44528E−13 | 6.85489E−11 |
| Ssr4 | 0.28033814 | 9.653692829 | 51.63120088 | 6.69688E−13 | 7.07593E−11 |
| Rps10 | 0.137124331 | 11.22177397 | 51.58076967 | 6.87113E−13 | 7.2129E−11 |
| Pomp | 0.278699623 | 9.701368752 | 51.44364086 | 7.36823E−13 | 7.68482E−11 |
| Ifitm2 | 0.480316373 | 10.68419752 | 51.28794721 | 7.97639E−13 | 8.25903E−11 |
| Rgs10 | 0.343212205 | 9.631731346 | 51.27700924 | 8.02095E−13 | 8.25903E−11 |
| Il2rg | −0.273364109 | 10.13353391 | 51.09215035 | 8.813E−13 | 9.01715E−11 |
| Tbx21 | −0.476153418 | 9.277692364 | 50.63469984 | 1.1126E−12 | 1.13122E−10 |
| Gcnt2 | −0.470647546 | 9.218058561 | 50.41597291 | 1.24378E−12 | 1.25668E−10 |
| Tesc | 0.35234916 | 9.483450902 | 49.94337593 | 1.58247E−12 | 1.58896E−10 |
| Slc39a1 | −0.433417379 | 9.215153753 | 49.92774503 | 1.59513E−12 | 1.59178E−10 |
| Tnfsf8 | 0.525733735 | 10.01783487 | 49.6323025 | 1.85435E−12 | 1.83911E−10 |
| Rpl11 | 0.110213367 | 11.70901858 | 49.60546792 | 1.87989E−12 | 1.85307E−10 |
| Tbca | 0.241332603 | 9.75478561 | 49.50794918 | 1.9757E−12 | 1.9357E−10 |
| Stk24 | −0.345711816 | 9.60853542 | 49.47575144 | 2.00839E−12 | 1.95588E−10 |
| Gngt2 | 0.417816178 | 9.363465198 | 49.3565518 | 2.1342E−12 | 2.06596E−10 |
| Ric1 | −0.453997651 | 9.254662379 | 49.34080833 | 2.1514E−12 | 2.07021E−10 |
| Commd8 | 0.336386603 | 9.438214185 | 48.22616976 | 3.79786E−12 | 3.63291E−10 |
| Eif3h | 0.175401684 | 10.29689224 | 48.11637516 | 4.01658E−12 | 3.81953E−10 |
| Sesn3 | 0.345251994 | 9.451624571 | 48.0760595 | 4.10002E−12 | 3.87608E−10 |
| Tacc3 | −0.330180442 | 9.190019982 | 47.8217631 | 4.66782E−12 | 4.3872E−10 |
| Lsm3 | −0.397355218 | 9.359397052 | 47.76572348 | 4.80317E−12 | 4.48832E−10 |
| Acot7 | 0.29321749 | 9.577129276 | 47.63640279 | 5.13069E−12 | 4.76682E−10 |
| Ccna2 | −0.368001411 | 9.19373092 | 47.55051727 | 5.36046E−12 | 4.95184E−10 |
| Tuba1c | −0.469993277 | 9.281183564 | 46.81782197 | 7.79017E−12 | 7.15545E−10 |
| Prelid1 | 0.27790907 | 9.693674656 | 46.79339374 | 7.88789E−12 | 7.20427E−10 |
| Gdi2 | 0.236813278 | 9.887621903 | 46.18643907 | 1.07519E−11 | 9.76486E−10 |
| Ldha | 0.212909741 | 10.35598102 | 46.03661224 | 1.16064E−11 | 1.0482E−09 |
| Cd74 | −0.739270074 | 9.434236295 | 46.00417217 | 1.18001E−11 | 1.05978E−09 |
| Supt16 | −0.441561553 | 9.269927603 | 45.92148251 | 1.23089E−11 | 1.09937E−09 |
| Got1 | 0.405705948 | 9.855701415 | 45.77574039 | 1.32596E−11 | 1.17777E−09 |
| Fxyd5 | 0.163739145 | 10.88897458 | 45.59890503 | 1.45123E−11 | 1.282E−09 |
| Sh2d2a | 0.289479061 | 9.84635475 | 45.32644577 | 1.66782E−11 | 1.46532E−09 |
| Zfp36l1 | −0.418870473 | 9.536622714 | 45.19149366 | 1.78679E−11 | 1.56137E−09 |
| Phlda1 | −0.539847883 | 9.44085361 | 45.17023117 | 1.8063E−11 | 1.56993E−09 |
| Notch2 | −0.425992475 | 9.219652512 | 45.03790208 | 1.93257E−11 | 1.6707E−09 |
| Gpi1 | 0.23829591 | 9.778165149 | 44.92930737 | 2.04278E−11 | 1.74728E−09 |
| Pdcd1 | −0.568864329 | 9.244437101 | 44.93462396 | 2.03724E−11 | 1.74728E−09 |
| Pnrc1 | −0.217943097 | 10.57790653 | 44.82469758 | 2.15487E−11 | 1.83346E−09 |
| Rpl10a | 0.138336371 | 11.14215396 | 44.52377247 | 2.51287E−11 | 2.12686E−09 |
| AW112010 | 0.227357928 | 11.38324175 | 44.21979772 | 2.93498E−11 | 2.47119E−09 |
| Plekhf1 | −0.295061063 | 9.179229479 | 44.15833723 | 3.02859E−11 | 2.5368E−09 |
| Gstm5 | −0.448209177 | 9.202000005 | 44.13230489 | 3.06914E−11 | 2.55751E−09 |
| Tuba4a | −0.412258234 | 9.389408567 | 43.88249634 | 3.48693E−11 | 2.89076E−09 |
| Ube2s | −0.268614956 | 10.0029558 | 43.69026594 | 3.8468E−11 | 3.15672E−09 |
| Tagap | −0.466944977 | 9.424808628 | 43.69120062 | 3.84497E−11 | 3.15672E−09 |
| Nt5e | 0.416621625 | 9.297374279 | 43.63415359 | 3.9587E−11 | 3.23214E−09 |
| Cd27 | −0.572422105 | 9.34285757 | 43.59576726 | 4.03711E−11 | 3.2796E−09 |
| Hjurp | −0.393196909 | 9.227444004 | 43.52899827 | 4.17723E−11 | 3.37646E−09 |
| Cdkn2d | −0.415706203 | 9.369245485 | 43.44303379 | 4.36483E−11 | 3.51054E−09 |
| Itgb1 | −0.592025611 | 9.299262307 | 43.33380996 | 4.61539E−11 | 3.69369E−09 |
| Cd160 | −0.650849788 | 9.334772106 | 43.31831197 | 4.65209E−11 | 3.70472E−09 |
| Prdx2 | 0.285220158 | 9.529013404 | 43.19820628 | 4.9466E−11 | 3.91399E−09 |
| Dnajc15 | 0.238508998 | 9.893117104 | 43.19161067 | 4.9633E−11 | 3.91399E−09 |
| Cd69 | −0.413066046 | 9.886545576 | 43.00176598 | 5.46905E−11 | 4.29187E−09 |
| Gnb2l1 | 0.158004594 | 10.54003804 | 42.81387557 | 6.02036E−11 | 4.67909E−09 |
| Slamf6 | 0.449149176 | 9.319040639 | 42.81726381 | 6.00994E−11 | 4.67909E−09 |
| Smc4 | −0.429041212 | 9.387774585 | 42.7743266 | 6.14331E−11 | 4.75181E−09 |
| Insig1 | −0.434558342 | 9.37348859 | 42.70816037 | 6.35466E−11 | 4.89188E−09 |
| Lag3 | −0.697350346 | 9.272207901 | 42.56198867 | 6.84773E−11 | 5.24646E−09 |
| Ms4a4b | 0.223679571 | 10.7518482 | 42.36461224 | 7.57483E−11 | 5.77616E−09 |
| Pttg1 | −0.391336105 | 9.448089255 | 42.14887763 | 8.45825E−11 | 6.41953E−09 |
| Stat1 | 0.341265519 | 9.58026255 | 42.13598362 | 8.5142E−11 | 6.4318E−09 |
| Hsph1 | −0.497945951 | 9.472478285 | 42.07543816 | 8.78193E−11 | 6.6032E−09 |
| Nrip1 | −0.403101209 | 9.582631136 | 41.82131157 | 1.00007E−10 | 7.48481E−09 |
| Atp5j | 0.205249314 | 9.978678584 | 41.68575303 | 1.07187E−10 | 7.9852E−09 |
| Nucks1 | −0.385882183 | 9.378591222 | 41.51091348 | 1.17215E−10 | 8.69217E−09 |
| Rps20 | 0.152105258 | 11.06851844 | 41.49249368 | 1.18324E−10 | 8.69469E−09 |
| Luc7l2 | −0.277739676 | 9.665593924 | 41.49974923 | 1.17886E−10 | 8.69469E−09 |
| Arpc1b | 0.161633259 | 10.55363559 | 41.32194002 | 1.29112E−10 | 9.44442E−09 |
| Ddx5 | −0.162093211 | 11.1992476 | 41.19466182 | 1.37798E−10 | 1.00345E−08 |
| Nusap1 | −0.445345836 | 9.207136218 | 41.16536981 | 1.39879E−10 | 1.01403E−08 |
| Slfn1 | 0.377286016 | 9.289471571 | 40.95793788 | 1.55541E−10 | 1.12253E−08 |
| Cytip | 0.210964863 | 10.1883862 | 40.8692185 | 1.62765E−10 | 1.16945E−08 |
| Atf3 | −0.562020803 | 9.29319516 | 40.67024137 | 1.80211E−10 | 1.28907E−08 |
| Ndufa13 | 0.175764796 | 10.09895175 | 40.4790137 | 1.98739E−10 | 1.41534E−08 |
| Slfn2 | 0.241009028 | 10.08513049 | 40.44035 | 2.02711E−10 | 1.43729E−08 |
| Amica1 | −0.49791256 | 9.335281825 | 40.34320419 | 2.13045E−10 | 1.50397E−08 |
| Rsrp1 | −0.252392032 | 10.00279565 | 40.30324944 | 2.17447E−10 | 1.52837E−08 |
| Eif3m | 0.213365247 | 9.796143519 | 40.14217018 | 2.36136E−10 | 1.65254E−08 |
| Inpp4a | −0.39180413 | 9.237585118 | 40.03084006 | 2.49985E−10 | 1.74192E−08 |
| Bcl11b | −0.360053334 | 9.518587772 | 39.49937961 | 3.28164E−10 | 2.27686E−08 |
| Id2 | −0.297282362 | 10.6456213 | 39.45801276 | 3.3519E−10 | 2.31567E−08 |
| Rras2 | 0.375221763 | 9.260363101 | 39.37014601 | 3.50617E−10 | 2.41194E−08 |
| Vmp1 | −0.333230189 | 9.490487697 | 39.25674927 | 3.71582E−10 | 2.54533E−08 |
| Plac8 | 0.394314739 | 10.56879742 | 39.04923729 | 4.13251E−10 | 2.81882E−08 |
| Iqgap1 | −0.285882286 | 9.634436107 | 38.96640363 | 4.31162E−10 | 2.90879E−08 |
| Klf13 | −0.313082682 | 9.695558545 | 38.96654438 | 4.31131E−10 | 2.90879E−08 |
| Cd226 | −0.412885191 | 9.268596107 | 38.96334564 | 4.31838E−10 | 2.90879E−08 |
| Smdt1 | 0.229451251 | 9.710629839 | 38.94863378 | 4.35105E−10 | 2.91736E−08 |
| Birc5 | −0.64959892 | 9.251453099 | 38.94139823 | 4.3672E−10 | 2.91736E−08 |
| Ap2s1 | 0.279837623 | 9.49800778 | 38.89100224 | 4.48141E−10 | 2.98134E−08 |
| Cenpe | −0.2171381 | 9.175299278 | 38.84242197 | 4.59434E−10 | 3.04394E−08 |
| Cd7 | −0.654275535 | 9.494173391 | 38.78759993 | 4.7252E−10 | 3.11786E−08 |
| Smc2 | −0.461435056 | 9.240564708 | 38.73553039 | 4.85294E−10 | 3.18913E−08 |
| Psma6 | 0.26537951 | 9.550367808 | 38.67878428 | 4.9961E−10 | 3.26992E−08 |
| Ly6c2 | −0.81589776 | 9.265993835 | 38.62656686 | 5.13157E−10 | 3.34504E−08 |
| Erdr1 | −0.417244234 | 9.351156146 | 38.61788986 | 5.15443E−10 | 3.34645E−08 |
| Rpl37 | 0.099363637 | 12.07300492 | 38.38701049 | 5.80173E−10 | 3.75163E−08 |
| Ubl3 | 0.283805464 | 9.565982019 | 38.27260434 | 6.15203E−10 | 3.9623E−08 |
| Psmb1 | 0.163974308 | 10.16664399 | 38.2135354 | 6.3411E−10 | 4.06787E−08 |
| H2-DMa | 0.355376414 | 9.273410275 | 37.58887051 | 8.73414E−10 | 5.58087E−08 |
| Stmn1 | −0.820204514 | 9.430119458 | 37.47441949 | 9.26198E−10 | 5.89485E−08 |

TABLE 3H
LPL-7 vs. All

| GeneSymbol | logFC | logCPM | LR | PValue | FDR |
|---|---|---|---|---|---|
| Eomes | 3.2798103 | 9.215511584 | 3251.147989 | 0 | 0 |
| Gzmk | 3.190153811 | 9.363664177 | 2419.333478 | 0 | 0 |
| Cd27 | 2.373366823 | 9.342839542 | 1206.34871 | 2.5442E−264 | 1.371E−260 |
| Furin | −3.348783874 | 10.38135368 | 1145.327777 | 4.6487E−251 | 1.8788E−247 |
| Ifitm2 | −3.790176258 | 10.6842835 | 1115.433989 | 1.4602E−244 | 4.7212E−241 |
| Cxcr6 | −2.192953647 | 10.38993282 | 898.5245923 | 2.0538E−197 | 5.5336E−194 |
| Ccl5 | 1.940472599 | 13.31106087 | 868.7682803 | 6.0444E−191 | 1.3959E−187 |
| Plac8 | −2.710532413 | 10.56890498 | 770.0228847 | 1.7774E−169 | 3.5916E−166 |
| Esm1 | 2.269358847 | 9.307909321 | 724.0160454 | 1.7922E−159 | 3.2192E−156 |
| Gm43291 | 1.497725884 | 9.168153577 | 705.4050464 | 1.9969E−155 | 3.2281E−152 |
| Sh3bgrl3 | −0.862038487 | 11.24482102 | 648.2472167 | 5.3741E−143 | 7.898E−140 |
| Tnfsf8 | −3.028860279 | 10.01788231 | 633.226239 | 9.9345E−140 | 1.3383E−136 |
| Sh2d1a | 1.383466761 | 9.507414541 | 592.0909227 | 8.7924E−131 | 1.0934E−127 |
| Bend4 | 1.324976481 | 9.167728233 | 556.482621 | 4.8952E−123 | 5.6526E−120 |
| Rgs16 | 1.955281568 | 9.196903871 | 517.915318 | 1.2025E−114 | 1.296E−111 |
| Tmsb4x | −0.452472001 | 14.24921403 | 481.1750869 | 1.1859E−106 | 1.1982E−103 |
| Ly6e | −0.861719259 | 10.76647189 | 465.704428 | 2.7576E−103 | 2.6223E−100 |
| Tmsb10 | −0.499708274 | 12.94246496 | 462.9150501 | 1.1157E−102 | 1.002E−99 |
| Rpa2 | 1.383564395 | 9.392679857 | 455.3472281 | 4.948E−101 | 4.20993E−98 |
| Ifitm3 | −2.675579011 | 9.717470428 | 450.7042073 | 5.0683E−100 | 4.09669E−97 |
| Nkg7 | 0.867977864 | 11.21019133 | 448.0411278 | 1.925E−99 | 1.48185E−96 |
| Tox | 1.504061266 | 9.202103104 | 416.0140164 | 1.79909E−92 | 1.322E−89 |
| Cd52 | −0.719754738 | 11.79358625 | 408.008358 | 9.94623E−91 | 6.9909E−88 |
| Emb | −1.227465129 | 10.25097718 | 388.8176348 | 1.4972E−86 | 1.00849E−83 |
| Chchd10 | 1.514895153 | 9.239742304 | 388.0906979 | 2.15545E−86 | 1.3938E−83 |
| Pim1 | −1.140164893 | 10.82570706 | 385.4996796 | 7.89981E−86 | 4.91186E−83 |
| Lgals1 | −0.966904165 | 11.62770282 | 377.1045116 | 5.31326E−84 | 3.18126E−81 |
| Vim | −1.164303346 | 10.47346981 | 371.6841636 | 8.04455E−83 | 4.64458E−80 |
| Cldnd1 | −1.594626701 | 9.794944744 | 356.8336127 | 1.37741E−79 | 7.67836E−77 |
| Rgs10 | −1.522269541 | 9.631716545 | 350.6347051 | 3.08265E−78 | 1.66114E−75 |
| Actn2 | −1.902931209 | 9.563357125 | 347.3787198 | 1.57749E−77 | 8.22635E−75 |
| Cxcr5 | 1.132676561 | 9.169334986 | 340.3476 | 5.36006E−76 | 2.70784E−73 |
| Lgals3 | −1.631074577 | 10.20017765 | 340.2629549 | 5.59247E−76 | 2.73963E−73 |
| Capg | −1.43890794 | 9.809387956 | 337.9741008 | 1.7623E−75 | 8.37921E−73 |
| Rpl41 | −0.313152443 | 12.79521848 | 320.3014113 | 1.24522E−71 | 5.75151E−69 |
| AA467197 | −1.792859182 | 9.471356876 | 313.3817233 | 4.00453E−70 | 1.79826E−67 |
| Tapbpl | 1.233104394 | 9.305800519 | 312.4747442 | 6.31139E−70 | 2.75757E−67 |
| Ly6a | −1.060460377 | 10.374912 | 311.9103347 | 8.37674E−70 | 3.56364E−67 |
| Il18r1 | −1.528906795 | 9.526772285 | 307.7518085 | 6.74503E−69 | 2.7959E−66 |
| Saraf | 0.698107441 | 10.21648074 | 302.7946342 | 8.10819E−68 | 3.27693E−65 |
| Asb2 | −1.706305638 | 9.480765257 | 301.6467395 | 1.44213E−67 | 5.68623E−65 |
| Itgb7 | −1.03195597 | 9.956571795 | 298.2957969 | 7.74575E−67 | 2.98137E−64 |
| Rasgef1a | 0.954832896 | 9.166122273 | 293.4389939 | 8.85616E−66 | 3.32951E−63 |
| Klrg1 | 1.479355795 | 9.202811068 | 292.035209 | 1.79105E−65 | 6.58048E−63 |
| Tmem176b | −1.947455291 | 9.448088308 | 283.3636793 | 1.3887E−63 | 4.98882E−61 |
| Tnfsf11 | −1.871570551 | 9.547025163 | 282.4665441 | 2.17822E−63 | 7.65502E−61 |
| Dcp1b | 1.217734056 | 9.182915755 | 273.4090354 | 2.05089E−61 | 7.05418E−59 |
| Rps27 | 0.343182787 | 13.23487563 | 268.9950753 | 1.87901E−60 | 6.32836E−58 |
| Fxyd5 | −0.561431404 | 10.88896842 | 265.4526332 | 1.11178E−59 | 3.66797E−57 |
| H2afy | −0.896629741 | 9.930098566 | 254.1059628 | 3.30622E−57 | 1.06897E−54 |
| Txk | 1.1165592 | 9.335401088 | 250.682348 | 1.84367E−56 | 5.84406E−54 |
| Gm2a | −1.254112994 | 9.58187137 | 244.1366839 | 4.92896E−55 | 1.53234E−52 |
| Cd2 | 0.602040611 | 10.31974063 | 234.8069914 | 5.33413E−53 | 1.62701E−50 |
| Irf7 | 1.233359958 | 9.309194937 | 227.5376027 | 2.05288E−51 | 6.1457E−49 |
| Lag3 | 1.648447237 | 9.27219078 | 227.0251467 | 2.65538E−51 | 7.80488E−49 |
| Rpl29 | −0.426163608 | 11.21429155 | 226.6636948 | 3.18389E−51 | 9.1912E−49 |
| S100a6 | −0.954302844 | 10.99625671 | 219.2973162 | 1.28724E−49 | 3.65078E−47 |
| Ret | 0.739017916 | 9.163901909 | 214.7890991 | 1.23901E−48 | 3.45342E−46 |
| Tmem176a | −1.728150993 | 9.404487704 | 212.7090541 | 3.52245E−48 | 9.65151E−46 |
| Ccr5 | 1.01474002 | 9.514413429 | 212.4623074 | 3.98726E−48 | 1.0743E−45 |
| Klrk1 | −1.632071328 | 9.564474145 | 206.9732412 | 6.28407E−47 | 1.66538E−44 |
| Wdr95 | 0.858966671 | 9.168703843 | 206.6330617 | 7.45528E−47 | 1.9439E−44 |
| Pde4b | −1.037180552 | 9.658605719 | 206.3101232 | 8.76851E−47 | 2.25003E−44 |
| Neurl3 | −1.11843401 | 9.61965738 | 204.6604978 | 2.00846E−46 | 5.07325E−44 |
| Cd40lg | −1.325579191 | 9.46906787 | 203.2747273 | 4.02941E−46 | 1.00214E−43 |
| H2-Q7 | 0.485660268 | 10.62100119 | 202.3256105 | 6.49151E−46 | 1.59003E−43 |
| Dnajc15 | −0.810147548 | 9.893127081 | 201.7430432 | 8.69899E−46 | 2.09892E−43 |
| Gzma | −2.279019802 | 10.72577583 | 199.2659566 | 3.02008E−45 | 7.17979E−43 |
| Sub1 | 0.447317923 | 11.14878706 | 198.1250066 | 5.35805E−45 | 1.25534E−42 |
| Tnfrsf4 | −0.896219752 | 10.62229818 | 189.4901987 | 4.10781E−43 | 9.4867E−41 |
| Ramp3 | −1.526846449 | 9.936866207 | 184.7197481 | 4.51835E−42 | 1.02878E−39 |
| Il10ra | 0.940599026 | 9.177837324 | 184.0570113 | 6.30468E−42 | 1.41558E−39 |
| Il12rb2 | −1.311113717 | 9.345966917 | 182.373313 | 1.46977E−41 | 3.25483E−39 |
| Rpl3 | −0.276118955 | 12.09253931 | 182.2652047 | 1.55185E−41 | 3.39017E−39 |
| Cd47 | −0.591482576 | 10.23577103 | 171.4400476 | 3.58636E−39 | 7.73027E−37 |
| Nr4a2 | 0.94692531 | 9.521282587 | 169.5951037 | 9.06986E−39 | 1.92925E−36 |
| Amica1 | −1.349724672 | 9.33525593 | 169.0337835 | 1.20282E−38 | 2.5253E−36 |
| Sema4a | 0.976179233 | 9.198632285 | 167.4717442 | 2.63868E−38 | 5.46883E−36 |
| Il7r | −0.953618116 | 9.753450222 | 166.8045235 | 3.69087E−38 | 7.55273E−36 |
| Ctsw | −0.77682414 | 9.943509554 | 166.4720465 | 4.36268E−38 | 8.81588E−36 |
| Icos | −0.730239997 | 10.25643633 | 164.2679456 | 1.32199E−37 | 2.63843E−35 |
| Actb | −0.746061335 | 13.40490306 | 163.2109404 | 2.24977E−37 | 4.43534E−35 |
| Satb1 | −0.948024097 | 9.757058763 | 160.1639583 | 1.0419E−36 | 2.02932E−34 |
| Tagln2 | −0.639877159 | 10.29867207 | 157.3789496 | 4.23005E−36 | 8.14082E−34 |
| Rplp0 | −0.262641417 | 12.38778931 | 157.2206301 | 4.58078E−36 | 8.71211E−34 |
| Gtf2i | 0.882631296 | 9.381266311 | 156.9371115 | 5.28314E−36 | 9.93107E−34 |
| Gpr18 | 0.970290866 | 9.406589754 | 155.8660296 | 9.05614E−36 | 1.68278E−33 |
| Klrc1 | −1.245205556 | 9.658438919 | 154.722076 | 1.61038E−35 | 2.95835E−33 |
| Sdcbp2 | 0.909955184 | 9.294064909 | 149.7774031 | 1.93916E−34 | 3.5223E−32 |
| Socs2 | −1.266440957 | 9.351659082 | 148.7076923 | 3.32227E−34 | 5.96754E−32 |
| mt-Co1 | −0.296844605 | 12.21554609 | 147.9872407 | 4.77442E−34 | 8.48167E−32 |
| Ahr | −1.187489037 | 9.328418697 | 147.556191 | 5.93126E−34 | 1.04222E−31 |
| Rora | −0.769975041 | 9.982396399 | 147.3734285 | 6.50277E−34 | 1.13036E−31 |
| Ctla4 | −0.752514198 | 10.15303783 | 143.5610512 | 4.43161E−33 | 7.62142E−31 |
| Gimap6 | 0.469906688 | 10.46581469 | 143.4578381 | 4.66797E−33 | 7.94341E−31 |
| Srgn | −0.392519863 | 11.37089272 | 143.1641996 | 5.41165E−33 | 9.11299E−31 |
| Ltb4r1 | −1.111665991 | 9.272000172 | 142.6946671 | 6.85474E−33 | 1.14241E−30 |
| Fam46a | −1.314561947 | 9.504335784 | 141.6679882 | 1.14942E−32 | 1.89607E−30 |
| Samd3 | 0.636378603 | 9.164604728 | 140.6602121 | 1.90916E−32 | 3.11753E−30 |
| Hk2 | −1.16240928 | 9.291515007 | 139.523414 | 3.38402E−32 | 5.47061E−30 |
| Itgae | −1.184859251 | 9.27549806 | 135.9237287 | 2.07344E−31 | 3.31873E−29 |
| Fasl | 0.707483582 | 9.594244101 | 135.8923984 | 2.10642E−31 | 3.33846E−29 |
| Rbm47 | 0.718009897 | 9.170928286 | 135.5181016 | 2.54338E−31 | 3.99188E−29 |
| Gpr34 | −1.061027039 | 9.256545758 | 134.3366515 | 4.61142E−31 | 7.1681E−29 |
| Eif1 | 0.260507074 | 11.85893183 | 134.2575124 | 4.79894E−31 | 7.38855E−29 |
| Gnas | 0.39392693 | 10.37735049 | 133.5033298 | 7.01649E−31 | 1.07008E−28 |
| Sp100 | 0.675383289 | 9.55366719 | 132.5975297 | 1.10731E−30 | 1.67297E−28 |
| Tbxa2r | 0.585759135 | 9.164267126 | 132.1983671 | 1.35391E−30 | 2.02661E−28 |
| Nt5e | −1.088041733 | 9.297376625 | 132.0590116 | 1.45237E−30 | 2.15404E−28 |
| Gxylt1 | 0.864574803 | 9.199924509 | 131.8738591 | 1.59435E−30 | 2.34311E−28 |
| Asap1 | 0.888126055 | 9.229868598 | 130.5342162 | 3.13089E−30 | 4.55981E−28 |
| Il2ra | −1.040299441 | 9.266106978 | 129.9752671 | 4.14915E−30 | 5.98886E−28 |
| Rpl10 | −0.310792871 | 11.48660702 | 129.9264066 | 4.25256E−30 | 6.08379E−28 |
| Fam110a | −0.964708157 | 9.494264974 | 129.6194418 | 4.96378E−30 | 7.03899E−28 |
| Cbx4 | 0.781047544 | 9.354636358 | 128.9857168 | 6.83082E−30 | 9.60235E−28 |
| Slc3a2 | 0.511278475 | 10.08968291 | 128.1171727 | 1.05808E−29 | 1.47457E−27 |
| Itgad | 0.535714085 | 9.161955942 | 126.8819115 | 1.97163E−29 | 2.72422E−27 |
| Tnfaip8 | −0.821651414 | 9.630233952 | 126.0811572 | 2.95162E−29 | 4.04372E−27 |
| Nebl | −1.069381966 | 9.313919832 | 124.5341642 | 6.43614E−29 | 8.74342E−27 |
| Rpsa | −0.277280078 | 11.60015063 | 123.8483581 | 9.09342E−29 | 1.22503E−26 |
| Epsti1 | −0.651296117 | 9.946025493 | 123.5305898 | 1.06728E−28 | 1.42592E−26 |
| Cst7 | 0.771669339 | 9.361569648 | 123.2695875 | 1.21733E−28 | 1.61306E−26 |
| H2-T22 | 0.511037144 | 9.836593368 | 122.1981069 | 2.08902E−28 | 2.74561E−26 |
| Klf3 | 0.838002162 | 9.422127484 | 120.9469914 | 3.9248E−28 | 5.1168E−26 |
| Cd96 | −0.884171126 | 9.446982611 | 120.5563123 | 4.77908E−28 | 6.18068E−26 |
| Ifitm1 | −2.559027388 | 9.599684322 | 118.0524471 | 1.68863E−27 | 2.16654E−25 |
| Cxcr4 | 0.877743485 | 9.203511935 | 117.4615351 | 2.27469E−27 | 2.89548E−25 |
| Dnase1l3 | 0.522754123 | 9.161900584 | 117.0414103 | 2.81136E−27 | 3.55066E−25 |
| Ddit3 | 0.779019377 | 9.376075281 | 116.2467391 | 4.19693E−27 | 5.25951E−25 |
| Plscr1 | −0.95795568 | 9.521575487 | 115.5912639 | 5.84083E−27 | 7.26329E−25 |
| Tspan3 | 0.784843484 | 9.325091853 | 114.3100538 | 1.11446E−26 | 1.3753E−24 |
| Klrd1 | −1.164056439 | 9.372657862 | 113.7518146 | 1.47683E−26 | 1.80867E−24 |
| Rps19 | −0.246604045 | 12.57926856 | 113.2192993 | 1.93182E−26 | 2.34811E−24 |
| Btg2 | −0.527557012 | 10.53785684 | 112.9321685 | 2.23284E−26 | 2.69374E−24 |
| Sat1 | 0.656951889 | 9.537032997 | 111.897264 | 3.76314E−26 | 4.50629E−24 |
| Ngfrap1 | 0.646356558 | 9.495133067 | 111.7125177 | 4.13066E−26 | 4.91002E−24 |
| S100a4 | −1.018352282 | 9.866562997 | 111.1597843 | 5.45887E−26 | 6.44147E−24 |
| Nfkbia | −0.597111959 | 10.83361265 | 105.0791749 | 1.17352E−24 | 1.37472E−22 |
| Aprt | −0.701406151 | 9.637312921 | 104.6412923 | 1.46374E−24 | 1.70237E−22 |
| Susd3 | −0.901261435 | 9.36994386 | 104.5375265 | 1.54244E−24 | 1.78108E−22 |
| H1f0 | −1.071867051 | 9.429812238 | 104.4247351 | 1.63279E−24 | 1.87204E−22 |
| Pfn1 | −0.317456871 | 12.14558154 | 102.6778946 | 3.9432E−24 | 4.48914E−22 |
| H3f3b | 0.257055136 | 12.34434425 | 102.3962134 | 4.5457E−24 | 5.13886E−22 |
| A930011G23Rik | 0.339865641 | 9.15868181 | 102.3174094 | 4.73017E−24 | 5.31027E−22 |
| Gm8369 | 0.521666409 | 9.924627075 | 101.6077892 | 6.76786E−24 | 7.54547E−22 |
| Dkkl1 | −0.896564378 | 9.308701044 | 100.1636514 | 1.40311E−23 | 1.55361E−21 |
| Tm6sf1 | 0.797062802 | 9.205998818 | 99.8736915 | 1.62433E−23 | 1.78632E−21 |
| Bhlhe40 | −0.43201621 | 10.72350871 | 99.34549809 | 2.12079E−23 | 2.31654E−21 |
| Irf2bpl | 0.749110924 | 9.293163503 | 98.99862068 | 2.52678E−23 | 2.74147E−21 |
| Ccr2 | 0.622882557 | 9.897726382 | 98.98187498 | 2.54823E−23 | 2.74632E−21 |
| Irf2 | 0.698581849 | 9.326143404 | 98.03421665 | 4.11215E−23 | 4.40245E−21 |
| Atp5l | −0.352277104 | 10.47765534 | 97.58180026 | 5.16767E−23 | 5.49609E−21 |
| 2010111I01Rik | 0.659221506 | 9.402781177 | 96.19904171 | 1.03895E−22 | 1.09776E−20 |
| St8sia1 | −0.835504537 | 9.247015066 | 95.74994331 | 1.3035E−22 | 1.36834E−20 |
| Pdcd1 | 0.966655751 | 9.244430667 | 95.28801588 | 1.64607E−22 | 1.71679E−20 |
| Rps27rt | 0.36584719 | 11.16196987 | 95.19935627 | 1.72146E−22 | 1.78392E−20 |
| Krtcap2 | −0.449237684 | 10.06413459 | 95.17864624 | 1.73957E−22 | 1.7912E−20 |
| H2-Q10 | 0.746706806 | 9.219532223 | 94.78298372 | 2.12444E−22 | 2.17365E−20 |
| Smco4 | −0.864745743 | 9.324712373 | 94.70819526 | 2.20624E−22 | 2.24315E−20 |
| Itm2b | −0.325043857 | 10.97659803 | 94.43754827 | 2.52948E−22 | 2.55573E−20 |
| Trps1 | 0.807993435 | 9.24785037 | 94.38115316 | 2.60258E−22 | 2.61325E−20 |
| Ehd1 | −0.785381669 | 9.471478828 | 94.29520457 | 2.71808E−22 | 2.71238E−20 |
| Lcp1 | −0.445197114 | 10.29115797 | 93.45936544 | 4.14626E−22 | 4.11218E−20 |
| Ccl4 | 1.798057092 | 9.567483078 | 93.36095469 | 4.35763E−22 | 4.29545E−20 |
| Itgb2 | 0.557196956 | 9.575080932 | 93.16885995 | 4.80177E−22 | 4.70457E−20 |
| Pttg1 | 0.665331536 | 9.448095727 | 93.09752183 | 4.978E−22 | 4.81883E−20 |
| Fubp3 | 0.676573702 | 9.185256957 | 93.09812625 | 4.97648E−22 | 4.81883E−20 |
| Ly6g5b | −0.772528223 | 9.223289292 | 92.75608537 | 5.91533E−22 | 5.69209E−20 |
| Wipf3 | 0.26176696 | 9.158140644 | 92.24996768 | 7.63914E−22 | 7.30736E−20 |
| AB124611 | 0.682659164 | 9.180450532 | 92.21780357 | 7.76431E−22 | 7.3834E−20 |
| Asap2 | 0.462878352 | 9.163205905 | 92.17975453 | 7.91504E−22 | 7.48272E−20 |
| Wbp5 | 0.710373707 | 9.336227139 | 92.08583153 | 8.29975E−22 | 7.8008E−20 |
| Lax1 | 0.750516768 | 9.205901785 | 91.84961807 | 9.35199E−22 | 8.73898E−20 |
| mt-Co3 | −0.242677521 | 12.09938987 | 91.67605628 | 1.02093E−21 | 9.48524E−20 |
| Gm12840 | −0.842973953 | 10.16274436 | 91.32502726 | 1.21909E−21 | 1.12616E−19 |
| Ostf1 | −0.48303019 | 10.18387303 | 91.04103711 | 1.40723E−21 | 1.29257E−19 |
| Acot7 | −0.644244133 | 9.577154168 | 90.9245269 | 1.49258E−21 | 1.36322E−19 |
| Timp2 | −0.865419253 | 9.295780623 | 90.80210372 | 1.58784E−21 | 1.44208E−19 |
| Rexo2 | −0.732259687 | 9.465193175 | 90.57002666 | 1.78545E−21 | 1.61249E−19 |
| Abhd17c | −0.839404611 | 9.339494063 | 89.96047193 | 2.42966E−21 | 2.18211E−19 |
| Ubc | 0.3685984 | 11.04999023 | 89.59143681 | 2.92789E−21 | 2.61504E−19 |
| Hspe1 | 0.538143649 | 10.27531776 | 89.36763916 | 3.27856E−21 | 2.91215E−19 |
| Ms4a6b | 0.412280945 | 10.19491628 | 89.30355751 | 3.3865E−21 | 2.99159E−19 |
| Klk8 | −0.677530964 | 9.559198221 | 88.48589784 | 5.11986E−21 | 4.49824E−19 |
| Prkch | 0.58070113 | 9.449515994 | 86.82690444 | 1.18447E−20 | 1.03504E−18 |
| Syf2 | 0.443448656 | 9.733081568 | 86.7777254 | 1.21429E−20 | 1.05539E−18 |
| Prdx6 | −0.457748392 | 10.19721197 | 86.54540808 | 1.36565E−20 | 1.1806E−18 |
| Adam8 | −0.807293103 | 9.247155567 | 85.89615093 | 1.89638E−20 | 1.63068E−18 |
| Ldlrad4 | −0.879974484 | 9.298455799 | 84.9562291 | 3.05044E−20 | 2.60917E−18 |
| Rcbtb2 | −0.877505664 | 9.39898186 | 84.63477143 | 3.58898E−20 | 3.05365E−18 |
| Gramd1a | −0.736031909 | 9.45889775 | 84.52414815 | 3.79551E−20 | 3.21247E−18 |
| Socs3 | 0.669638314 | 9.492279241 | 84.41671087 | 4.00746E−20 | 3.3742E−18 |
| Kremen2 | 0.614419032 | 9.177276538 | 83.90371615 | 5.19468E−20 | 4.35115E−18 |
| Ftl1 | 0.30419987 | 10.82047194 | 83.72493817 | 5.68634E−20 | 4.73741E−18 |
| Fyn | 0.561444187 | 9.486733177 | 83.7151954 | 5.71443E−20 | 4.73741E−18 |
| Npc2 | 0.410579927 | 9.848489901 | 83.70139601 | 5.75446E−20 | 4.74625E−18 |
| Gcnt2 | −0.743016267 | 9.218046584 | 83.23349557 | 7.29117E−20 | 5.9832E−18 |
| Rpl13 | −0.173445727 | 12.63772739 | 83.18432332 | 7.47482E−20 | 6.10292E−18 |
| Cxcr3 | 0.586788812 | 9.551365757 | 83.01615013 | 8.13857E−20 | 6.61147E−18 |
| Perp | 0.636271306 | 9.176884673 | 81.52799393 | 1.72797E−19 | 1.39671E−17 |
| Rps18 | −0.182969674 | 12.47333304 | 81.01252335 | 2.24292E−19 | 1.80393E−17 |
| H3f3a | −0.343723722 | 10.34149301 | 80.77491166 | 2.52949E−19 | 2.02434E−17 |
| Prr5 | −0.668175925 | 9.209950504 | 79.99053942 | 3.76207E−19 | 2.99594E−17 |
| Maf | 0.600777868 | 9.568684693 | 79.76182883 | 4.22374E−19 | 3.34711E−17 |
| 1700001O22Rik | 0.372137118 | 9.160348241 | 79.58478248 | 4.6197E−19 | 3.64303E−17 |
| Slco3a1 | −0.761871352 | 9.251168617 | 79.16254048 | 5.72045E−19 | 4.48916E−17 |
| Rbm3 | −0.335572075 | 10.73102924 | 78.7953872 | 6.88877E−19 | 5.3799E−17 |
| Cyb5a | −0.677604285 | 9.520917476 | 78.29568723 | 8.87153E−19 | 6.89506E−17 |
| Anxa1 | −0.915818021 | 9.278561592 | 77.85704634 | 1.10774E−18 | 8.56831E−17 |
| Gramd3 | 0.483169371 | 10.20641741 | 77.84616622 | 1.11386E−18 | 8.57461E−17 |
| Cd44 | −0.628062155 | 9.560090954 | 77.66490234 | 1.22092E−18 | 9.35419E−17 |
| Cd160 | −1.060008934 | 9.334762898 | 77.45982745 | 1.3545E−18 | 1.03287E−16 |
| Gata3 | −0.861175492 | 9.445565238 | 77.30607749 | 1.46415E−18 | 1.11124E−16 |
| Podnl1 | −0.768407408 | 9.399879407 | 76.93648778 | 1.76545E−18 | 1.33366E−16 |
| Gpr55 | −0.713985194 | 9.217387159 | 76.83201579 | 1.86136E−18 | 1.39957E−16 |
| Cd247 | 0.514152093 | 9.534122941 | 76.54776484 | 2.14951E−18 | 1.60875E−16 |
| Rps2 | −0.231179288 | 11.63572242 | 75.92273731 | 2.94984E−18 | 2.19756E−16 |
| Dapk2 | −0.812348541 | 9.267248742 | 75.61455589 | 3.4481E−18 | 2.55697E−16 |
| Bcl2a1d | −0.536568274 | 9.947945196 | 74.71398893 | 5.44092E−18 | 4.01634E−16 |
| Rpl35 | −0.266038037 | 11.18858014 | 74.15706214 | 7.21425E−18 | 5.30116E−16 |
| Ormdl3 | 0.662349433 | 9.242329201 | 73.61447025 | 9.49659E−18 | 6.94669E−16 |
| Rpl24 | −0.190402777 | 11.77226429 | 73.2218925 | 1.15863E−17 | 8.43716E−16 |
| Tesc | −0.660354554 | 9.483456771 | 73.0535519 | 1.26179E−17 | 9.14715E−16 |
| Cks2 | −0.777355745 | 9.525757792 | 72.98672918 | 1.30525E−17 | 9.41991E−16 |
| Gm12216 | 0.680896072 | 9.227687607 | 72.7754164 | 1.45275E−17 | 1.04379E−15 |
| Plek | 0.751449862 | 9.215821963 | 72.38413926 | 1.77132E−17 | 1.26704E−15 |
| Sh3glb1 | 0.450965438 | 9.599133015 | 72.35881311 | 1.7942E−17 | 1.27775E−15 |
| Malat1 | 0.278672905 | 14.6685717 | 72.26960928 | 1.87716E−17 | 1.33097E−15 |
| Rbpj | −0.711555719 | 9.385172047 | 71.94087461 | 2.21743E−17 | 1.56537E−15 |
| Ppib | −0.424361083 | 9.880940736 | 71.9313067 | 2.22821E−17 | 1.56614E−15 |
| mt-Atp6 | −0.201773503 | 12.47131233 | 71.76755318 | 2.42101E−17 | 1.69429E−15 |
| Sh2d2a | 0.447240574 | 9.846353728 | 71.46835454 | 2.81741E−17 | 1.9632E−15 |
| Cish | −0.69379779 | 9.408299131 | 71.04548685 | 3.49083E−17 | 2.42201E−15 |
| Hint1 | −0.384281305 | 10.08270698 | 70.72226399 | 4.11225E−17 | 2.84097E−15 |
| Cd74 | −1.106243236 | 9.434237857 | 70.69797546 | 4.16319E−17 | 2.86392E−15 |
| Lbh | 0.579804787 | 9.408456529 | 70.37740415 | 4.89777E−17 | 3.35497E−15 |
| Lpin1 | 0.632547096 | 9.197406237 | 69.60019426 | 7.26297E−17 | 4.95415E−15 |
| Hsp90ab1 | 0.345884126 | 11.19694983 | 69.53672628 | 7.50048E−17 | 5.09466E−15 |
| Ubb | 0.242830128 | 12.90471764 | 69.26272166 | 8.61832E−17 | 5.82945E−15 |
| Tm2d3 | 0.65663079 | 9.233563913 | 69.12669132 | 9.23372E−17 | 6.21968E−15 |
| Clic1 | −0.354187575 | 10.3255658 | 68.54893155 | 1.23768E−16 | 8.30222E−15 |
| Mxi1 | 0.61312214 | 9.308254267 | 68.4810021 | 1.28106E−16 | 8.55768E−15 |
| Kcnip3 | 0.214050413 | 9.158157362 | 68.20905605 | 1.47049E−16 | 9.78268E−15 |
| Rgs2 | −0.664820587 | 10.05989421 | 68.2006855 | 1.47674E−16 | 9.78403E−15 |
| Cd69 | −0.645530905 | 9.886453138 | 68.1516296 | 1.51394E−16 | 9.98955E−15 |
| Irf1 | 0.591854113 | 9.431031538 | 67.47468468 | 2.1341E−16 | 1.40243E−14 |
| Gcg | 0.648871754 | 9.162673096 | 67.15821837 | 2.50569E−16 | 1.63996E−14 |
| Arpc3 | −0.314794705 | 10.54009876 | 67.07583725 | 2.61261E−16 | 1.70304E−14 |
| Clint1 | −0.604253767 | 9.518647713 | 66.99244015 | 2.7255E−16 | 1.7695E−14 |
| Myl6 | −0.256992464 | 10.93122698 | 66.77019019 | 3.05077E−16 | 1.97275E−14 |
| Nfkbiz | −0.598905696 | 9.633433533 | 66.72696278 | 3.1184E−16 | 2.00845E−14 |
| Reep5 | −0.520338628 | 9.637155571 | 66.49848676 | 3.50162E−16 | 2.24632E−14 |
| Nme1 | −0.531092689 | 9.653532734 | 66.23423055 | 4.00398E−16 | 2.55843E−14 |
| Nck1 | 0.612718075 | 9.252796487 | 66.02666346 | 4.44864E−16 | 2.83137E−14 |
| Bcl2a1b | −0.398493382 | 10.39217608 | 65.1482998 | 6.94684E−16 | 4.40402E−14 |
| Gng5 | 0.34112214 | 9.899707496 | 64.67244017 | 8.84433E−16 | 5.58506E−14 |
| Cox5a | −0.369902109 | 10.08152668 | 64.56707148 | 9.33016E−16 | 5.86893E−14 |
| D1Ertd622e | 0.615527633 | 9.208393557 | 64.30341259 | 1.06661E−15 | 6.68325E−14 |
| Ube2b | 0.353641396 | 9.912283123 | 63.83320012 | 1.35412E−15 | 8.45202E−14 |
| Pnrc1 | 0.298835463 | 10.57783069 | 63.57012017 | 1.54759E−15 | 9.62243E−14 |
| Ier3 | 0.686673886 | 9.17570835 | 63.29753534 | 1.77727E−15 | 1.10082E−13 |
| Cyb5r3 | 0.589129593 | 9.274984141 | 62.76482846 | 2.32921E−15 | 1.43718E−13 |
| Sec62 | 0.390672881 | 9.756942892 | 62.73250459 | 2.36776E−15 | 1.4554E−13 |
| P2rx7 | −0.686106279 | 9.260764108 | 62.71139686 | 2.39327E−15 | 1.46551E−13 |
| Cd226 | −0.718795459 | 9.268586644 | 62.40322215 | 2.79865E−15 | 1.70728E−13 |
| Maff | −0.785642781 | 9.305243703 | 62.11356649 | 3.2421E−15 | 1.97037E−13 |
| Cir1 | 0.540883115 | 9.390079546 | 61.8636656 | 3.68079E−15 | 2.2286E−13 |
| Mier1 | −0.56737739 | 9.536740084 | 61.39838388 | 4.66193E−15 | 2.81212E−13 |
| Hvcn1 | −0.697052978 | 9.29925934 | 61.38224575 | 4.7003E−15 | 2.82472E−13 |
| Leprotl1 | 0.387799851 | 9.724295192 | 61.16177005 | 5.25725E−15 | 3.14773E−13 |
| Akap13 | −0.346613669 | 10.30878346 | 61.06204963 | 5.5304E−15 | 3.29906E−13 |
| Cables1 | 0.58247393 | 9.182534864 | 60.94449572 | 5.87068E−15 | 3.48917E−13 |
| Ubald2 | 0.334201988 | 10.62010958 | 60.89344557 | 6.02491E−15 | 3.56772E−13 |
| Ass1 | −0.613406117 | 9.534311369 | 60.42124224 | 7.65821E−15 | 4.51834E−13 |
| Fam104a | 0.504996169 | 9.38496573 | 60.18580376 | 8.63125E−15 | 5.05913E−13 |
| Atp5h | −0.278014591 | 10.35719028 | 60.18440409 | 8.63739E−15 | 5.05913E−13 |
| Dusp10 | −0.717056732 | 9.475530411 | 60.11639783 | 8.94103E−15 | 5.21808E−13 |
| 6330403K07Rik | 0.600891856 | 9.187821133 | 59.89795809 | 9.9905E−15 | 5.80959E−13 |
| mt-Cytb | −0.259020182 | 11.07894922 | 59.78485862 | 1.05814E−14 | 6.13116E−13 |
| Sepp1 | −0.723631668 | 9.293155529 | 59.50165472 | 1.22191E−14 | 7.0548E−13 |
| Il1r2 | −0.903532683 | 9.256685507 | 59.46822742 | 1.24285E−14 | 7.15012E−13 |
| Orai1 | 0.392968699 | 9.884602454 | 59.42944137 | 1.26758E−14 | 7.26658E−13 |
| Crtam | 0.457167582 | 9.163663097 | 59.2478005 | 1.39016E−14 | 7.94108E−13 |
| Eif5 | 0.382500506 | 9.823460355 | 59.19925072 | 1.42488E−14 | 8.11078E−13 |
| Actg1 | −0.24698467 | 12.09934301 | 59.02051658 | 1.56037E−14 | 8.85083E−13 |
| Vmp1 | 0.480500703 | 9.490450465 | 58.65631384 | 1.87765E−14 | 1.06133E−12 |
| Cd3g | −0.238281838 | 11.10939413 | 58.41952928 | 2.11778E−14 | 1.19289E−12 |
| Lysmd2 | −0.676383806 | 9.243342087 | 58.30789378 | 2.24143E−14 | 1.25816E−12 |
| Ccdc50 | −0.636888834 | 9.24053109 | 58.24392228 | 2.31551E−14 | 1.29524E−12 |
| Coro1a | −0.294730343 | 10.77307504 | 58.19082122 | 2.37886E−14 | 1.32609E−12 |
| Ctsd | 0.432871133 | 9.641722001 | 58.04221936 | 2.56552E−14 | 1.42523E−12 |
| Adam19 | 0.588396014 | 9.250767663 | 57.92386217 | 2.72462E−14 | 1.50843E−12 |
| Gm9844 | −0.531404913 | 9.544148899 | 57.76929535 | 2.94735E−14 | 1.62064E−12 |
| H2-Q6 | 0.39965633 | 9.740245985 | 57.7724562 | 2.94261E−14 | 1.62064E−12 |
| Rnf149 | −0.678390076 | 9.332967884 | 57.76244641 | 2.95763E−14 | 1.62078E−12 |
| Faim | 0.553709883 | 9.309636125 | 57.6673891 | 3.10407E−14 | 1.69528E−12 |
| Gstm5 | −0.595633706 | 9.20199523 | 57.63484148 | 3.15586E−14 | 1.71776E−12 |
| Ssr4 | −0.462687492 | 9.653712425 | 57.61772544 | 3.18344E−14 | 1.72696E−12 |
| Ppp1ca | −0.313444097 | 10.29798996 | 57.51174091 | 3.35968E−14 | 1.81647E−12 |
| Man1a | 0.591903753 | 9.262299772 | 57.36248846 | 3.62455E−14 | 1.94666E−12 |
| Ak2 | −0.559791812 | 9.46480863 | 57.36453397 | 3.62078E−14 | 1.94666E−12 |
| Hilpda | −0.610088711 | 9.667525426 | 57.29728897 | 3.74671E−14 | 2.00561E−12 |
| Hspd1 | 0.490465034 | 9.781069436 | 57.21625898 | 3.9043E−14 | 2.08307E−12 |
| Ifrd1 | −0.452953447 | 10.02590859 | 57.12858627 | 4.08229E−14 | 2.17086E−12 |
| Sptssa | −0.528455842 | 9.597747238 | 57.11895346 | 4.10233E−14 | 2.17437E−12 |
| Ero1l | −0.653981362 | 9.301167052 | 56.87647737 | 4.64064E−14 | 2.4453E−12 |
| Znrf1 | −0.625877517 | 9.40064809 | 56.87516065 | 4.64374E−14 | 2.4453E−12 |
| Slc15a3 | −0.645993624 | 9.22733484 | 56.72999974 | 4.9995E−14 | 2.62409E−12 |
| Snrpd3 | −0.513107614 | 9.547220839 | 56.57419645 | 5.41174E−14 | 2.83127E−12 |
| Nab1 | 0.476701688 | 9.481616428 | 56.13698376 | 6.75936E−14 | 3.5249E−12 |
| Tmbim4 | −0.620175868 | 9.33363574 | 55.75215867 | 8.2208E−14 | 4.27323E−12 |
| Ptpn6 | −0.655690323 | 9.338656784 | 55.65343816 | 8.64416E−14 | 4.47889E−12 |
| Thap3 | 0.571712939 | 9.265378945 | 55.63069262 | 8.74476E−14 | 4.51654E−12 |
| Son | 0.380723151 | 9.69657111 | 55.47166754 | 9.48158E−14 | 4.8815E−12 |
| Erp44 | 0.484253067 | 9.382705163 | 55.39965547 | 9.83537E−14 | 5.04757E−12 |
| H2-Q4 | 0.389723252 | 9.753745914 | 55.06953171 | 1.1634E−13 | 5.95177E−12 |
| Lmo1 | 0.219255613 | 9.158225061 | 55.00767127 | 1.2006E−13 | 6.1227E−12 |
| Prf1 | 0.305417186 | 9.16134179 | 54.73770604 | 1.37738E−13 | 7.00212E−12 |
| Sp110 | 0.488354994 | 9.437502438 | 54.636272 | 1.45034E−13 | 7.3499E−12 |
| Eif3h | −0.277769071 | 10.296933 | 54.59602322 | 1.48035E−13 | 7.47854E−12 |
| Itga1 | −0.499265851 | 9.19749427 | 54.25356867 | 1.76217E−13 | 8.87452E−12 |
| Pde4d | 0.552817694 | 9.266026673 | 54.1068785 | 1.89875E−13 | 9.53269E−12 |
| Atf3 | −0.817587319 | 9.293189291 | 54.0557667 | 1.94879E−13 | 9.75362E−12 |
| Ets1 | 0.28518737 | 10.24991887 | 53.90587312 | 2.10328E−13 | 1.04943E−11 |
| Ubash3b | 0.502003409 | 9.181348024 | 53.89976936 | 2.10982E−13 | 1.04946E−11 |
| Clec12a | −0.430462755 | 9.187014324 | 53.88824045 | 2.12224E−13 | 1.0524E−11 |
| Dgat1 | −0.55834131 | 9.778715844 | 53.80814124 | 2.21055E−13 | 1.09284E−11 |
| Cdc42ep3 | 0.575632949 | 9.28694131 | 53.17349427 | 3.05351E−13 | 1.50497E−11 |
| Hsp90aa1 | 0.420791665 | 10.76849068 | 53.14096424 | 3.1045E−13 | 1.52545E−11 |
| Kdm6b | −0.638583048 | 9.740481683 | 53.04665018 | 3.2572E−13 | 1.59563E−11 |
| Spcs2 | −0.360764471 | 9.914030831 | 52.97319432 | 3.38132E−13 | 1.65143E−11 |
| H2afz | −0.360541992 | 10.76566171 | 52.78471826 | 3.72186E−13 | 1.81228E−11 |
| Gm13546 | −0.513269881 | 9.199885349 | 52.69946318 | 3.88697E−13 | 1.88699E−11 |
| Rhoh | 0.473436285 | 9.501551957 | 52.55043386 | 4.1934E−13 | 2.02966E−11 |
| Gm14718 | −0.588453419 | 9.20570763 | 52.38609479 | 4.55941E−13 | 2.19367E−11 |
| Cd48 | −0.494282495 | 9.721189722 | 52.38800866 | 4.55497E−13 | 2.19367E−11 |
| Traf1 | −0.367189394 | 10.10520209 | 52.10848359 | 5.25175E−13 | 2.51183E−11 |
| Cd9 | 0.629962799 | 9.231253154 | 52.1093635 | 5.2494E−13 | 2.51183E−11 |
| Armc7 | 0.550696024 | 9.197060421 | 52.04413059 | 5.42672E−13 | 2.58786E−11 |
| Eef2 | −0.182985174 | 11.29634654 | 52.01630705 | 5.50416E−13 | 2.61707E−11 |
| Hnrnpdl | 0.406565088 | 9.556908077 | 51.93389098 | 5.74011E−13 | 2.72125E−11 |
| Cox6b1 | −0.298181628 | 10.15773008 | 51.53029184 | 7.05009E−13 | 3.33251E−11 |
| Anp32a | −0.368441055 | 9.87826882 | 51.36308833 | 7.67685E−13 | 3.61819E−11 |
| Il10 | 0.872476336 | 9.17451578 | 51.1391009 | 8.60471E−13 | 4.04371E−11 |
| Ndufs5 | −0.512031204 | 9.463767548 | 51.08750093 | 8.8339E−13 | 4.13939E−11 |
| Fam3c | −0.605630051 | 9.322868853 | 51.02033744 | 9.14139E−13 | 4.27109E−11 |
| Gna13 | −0.397146052 | 9.985113984 | 50.90425396 | 9.69832E−13 | 4.51185E−11 |
| Cpd | −0.473218402 | 9.194132269 | 50.90138385 | 9.71251E−13 | 4.51185E−11 |
| Gpr174 | 0.55340071 | 9.263821812 | 50.88606644 | 9.7886E−13 | 4.53417E−11 |
| Osr2 | 0.528324536 | 9.161148525 | 50.41408333 | 1.24498E−12 | 5.75037E−11 |
| Plxnc1 | 0.364392898 | 9.165511777 | 50.1943726 | 1.39247E−12 | 6.41328E−11 |
| Gnai2 | −0.334762855 | 10.17428897 | 50.13923682 | 1.43215E−12 | 6.57729E−11 |
| Cox4i1 | −0.221749475 | 10.68633283 | 50.06740638 | 1.48554E−12 | 6.80319E−11 |
| H2-K1 | 0.188614934 | 11.60998915 | 50.02198623 | 1.52033E−12 | 6.94284E−11 |
| Cytip | −0.328069424 | 10.18842314 | 50.00987741 | 1.52974E−12 | 6.94657E−11 |
| Nrn1 | 0.483613566 | 9.165074057 | 50.01143543 | 1.52853E−12 | 6.94657E−11 |
| Rab14 | 0.426761389 | 9.461449449 | 49.89200515 | 1.62445E−12 | 7.35599E−11 |
| Gimap7 | 0.313625671 | 10.07498565 | 49.73569548 | 1.75916E−12 | 7.94374E−11 |
| Yes1 | −0.604422292 | 9.221082612 | 49.55380987 | 1.93005E−12 | 8.69112E−11 |
| Cxcl10 | −0.914697883 | 9.358345131 | 49.37721533 | 2.11184E−12 | 9.48335E−11 |
| Gimap4 | 0.259745694 | 10.52499935 | 49.36824352 | 2.12152E−12 | 9.50043E−11 |
| Rps26 | −0.21575601 | 10.99404089 | 49.26863753 | 2.23203E−12 | 9.96766E−11 |
| Gabarapl2 | −0.350190949 | 9.899410329 | 48.56016717 | 3.20309E−12 | 1.42648E−10 |
| Aldoa | −0.304682663 | 10.23625965 | 48.51986093 | 3.26961E−12 | 1.4521E−10 |
| Mef2d | 0.532711694 | 9.220655304 | 48.44143467 | 3.40301E−12 | 1.50721E−10 |
| Tmbim6 | 0.267484208 | 10.08427654 | 48.41247816 | 3.45364E−12 | 1.52129E−10 |
| Rnf145 | 0.529781681 | 9.217590475 | 48.41300404 | 3.45271E−12 | 1.52129E−10 |
| Klk1b27 | 0.380972786 | 9.165727377 | 48.39798189 | 3.47926E−12 | 1.52689E−10 |
| Ckb | −0.660760349 | 9.33571335 | 48.39461397 | 3.48524E−12 | 1.52689E−10 |
| Rnf19b | −0.589896188 | 9.379257024 | 48.23892168 | 3.77324E−12 | 1.6486E−10 |
| Pld3 | 0.485699376 | 9.363216876 | 48.13576818 | 3.97705E−12 | 1.73297E−10 |
| Dennd5a | −0.564488698 | 9.227020583 | 47.77273449 | 4.78602E−12 | 2.07986E−10 |
| Cd68 | 0.374640404 | 9.164093483 | 47.73200657 | 4.88649E−12 | 2.11783E−10 |
| Ifng | −0.824643513 | 9.686644277 | 47.71134049 | 4.93827E−12 | 2.13455E−10 |
| Nod1 | 0.399719281 | 9.170204215 | 47.68415533 | 5.00722E−12 | 2.15858E−10 |
| Ndufa3 | −0.350060637 | 9.879747436 | 47.57998143 | 5.2805E−12 | 2.27033E−10 |
| Pdlim1 | −0.602115592 | 9.286492478 | 47.41649323 | 5.73977E−12 | 2.46125E−10 |
| Dnaja1 | 0.34905358 | 10.76370187 | 47.35226766 | 5.93095E−12 | 2.5365E−10 |
| Pnrc2 | 0.469933719 | 9.363165739 | 47.30698162 | 6.06956E−12 | 2.58893E−10 |
| Trpc1 | 0.383243845 | 9.166364579 | 47.20240867 | 6.40217E−12 | 2.72362E−10 |
| Cd84 | 0.499531179 | 9.348586397 | 47.08726344 | 6.78955E−12 | 2.88083E−10 |
| S100a13 | −0.354122862 | 9.897856812 | 46.79593256 | 7.87767E−12 | 3.33378E−10 |
| Hsph1 | 0.561731891 | 9.47244434 | 46.69789632 | 8.28179E−12 | 3.49565E−10 |
| Ccnd3 | −0.500150776 | 9.527417169 | 46.67505351 | 8.37889E−12 | 3.52742E−10 |
| Slc25a51 | −0.575066526 | 9.32805566 | 46.52560617 | 9.0429E−12 | 3.79708E−10 |
| Serpinb1a | −0.924931877 | 9.512638923 | 46.22319099 | 1.05521E−11 | 4.41929E−10 |
| Fdx1 | −0.61661336 | 9.293651654 | 46.1565072 | 1.09174E−11 | 4.56047E−10 |
| Fgl2 | −0.588978631 | 9.496125029 | 46.11929581 | 1.11267E−11 | 4.63594E−10 |
| Calm2 | 0.260443806 | 10.41347078 | 45.85998996 | 1.27014E−11 | 5.27842E−10 |
| Nrarp | 0.497355543 | 9.184041384 | 45.77903133 | 1.32373E−11 | 5.48703E−10 |
| Cfl1 | −0.209942284 | 11.11285008 | 45.74902911 | 1.34416E−11 | 5.55746E−10 |
| Gm8730 | −0.272607384 | 10.44469147 | 45.58287839 | 1.46315E−11 | 6.034E−10 |
| Rps9 | −0.140221794 | 11.99538144 | 45.57537937 | 1.46876E−11 | 6.04173E−10 |
| Dctpp1 | −0.584823203 | 9.308258583 | 45.45062678 | 1.56535E−11 | 6.42272E−10 |
| Myeov2 | −0.391999007 | 9.682148255 | 45.31640752 | 1.67639E−11 | 6.86088E−10 |
| Lsp1 | −0.305708206 | 10.32146671 | 45.30229843 | 1.68851E−11 | 6.89304E−10 |
| Ccl1 | −1.071553426 | 9.258105615 | 45.24994825 | 1.73425E−11 | 7.06194E−10 |
| Ccna2 | −0.408457876 | 9.193761792 | 45.04913135 | 1.92152E−11 | 7.80487E−10 |
| Klrc2 | −0.651905895 | 9.283426969 | 45.01296321 | 1.95734E−11 | 7.93043E−10 |
| Zfp36l2 | 0.427884779 | 9.814090687 | 44.98399358 | 1.98652E−11 | 8.0285E−10 |
| 2310031A07Rik | 0.362915586 | 9.165479785 | 44.90459464 | 2.06872E−11 | 8.33988E−10 |
| Lamb3 | 0.532127003 | 9.282561154 | 44.76329977 | 2.22351E−11 | 8.94163E−10 |
| Rpl10a | −0.187109705 | 11.1422209 | 44.63983869 | 2.36823E−11 | 9.49997E−10 |
| Tmem33 | 0.477950768 | 9.3077973 | 44.5371315 | 2.49579E−11 | 9.98685E−10 |
| Larp7 | 0.487803635 | 9.322608334 | 44.48668833 | 2.56093E−11 | 1.02222E−09 |
| Laptm5 | 0.221475236 | 10.49800706 | 44.38990272 | 2.69071E−11 | 1.07138E−09 |
| 2810417H13Rik | −1.100112922 | 9.337396993 | 44.380798 | 2.70326E−11 | 1.07373E−09 |
| Tnfaip8l2 | −0.584535976 | 9.325792226 | 44.31580305 | 2.79451E−11 | 1.10726E−09 |
| Plekha2 | 0.500523877 | 9.289919496 | 44.30617094 | 2.8083E−11 | 1.11E−09 |
| Ptma | −0.225883777 | 11.28824476 | 44.22970703 | 2.92016E−11 | 1.1514E−09 |
| E130308A19Rik | 0.31445378 | 9.162753622 | 44.16856614 | 3.01281E−11 | 1.18504E−09 |
| Nsmaf | 0.506430735 | 9.239663944 | 44.08781407 | 3.13969E−11 | 1.23195E−09 |
| Eif4a2 | 0.360471487 | 9.72382619 | 43.94614377 | 3.37537E−11 | 1.32122E−09 |
| Susd1 | 0.169765045 | 9.158248282 | 43.86105877 | 3.52534E−11 | 1.37658E−09 |
| Il4ra | −0.514186967 | 9.499579295 | 43.78225573 | 3.67018E−11 | 1.42969E−09 |
| Gas7 | 0.534018731 | 9.194255021 | 43.70792329 | 3.81225E−11 | 1.48146E−09 |
| Ncl | 0.278368496 | 10.07281784 | 43.57228777 | 4.08584E−11 | 1.58397E−09 |
| H2-DMa | −0.576877869 | 9.273413107 | 43.48277925 | 4.27707E−11 | 1.65414E−09 |
| Nfkb1 | −0.476422063 | 9.537085465 | 43.45398589 | 4.34047E−11 | 1.67167E−09 |
| Klhl6 | −0.579490641 | 9.316904493 | 43.45280579 | 4.34308E−11 | 1.67167E−09 |
| Rsu1 | −0.553456622 | 9.325990907 | 43.2691892 | 4.77036E−11 | 1.83177E−09 |
| Zfp36l1 | −0.538126975 | 9.536569365 | 43.14567424 | 5.08121E−11 | 1.94651E−09 |
| Myo1g | −0.502350092 | 9.439423987 | 42.90718154 | 5.73996E−11 | 2.19367E−09 |
| Bcl11b | 0.437395638 | 9.518527425 | 42.87577242 | 5.83286E−11 | 2.22391E−09 |
| Camk2n1 | 0.51765763 | 9.340518386 | 42.78669427 | 6.1046E−11 | 2.32205E−09 |
| Cox6c | −0.25524067 | 10.25790139 | 42.70764093 | 6.35635E−11 | 2.41213E−09 |
| Xcl1 | −1.217474518 | 9.40505696 | 42.44448966 | 7.2717E−11 | 2.75303E−09 |
| Ddx5 | 0.192028691 | 11.19922275 | 42.13065591 | 8.53743E−11 | 3.22467E−09 |
| Hic1 | −0.58737025 | 9.270379257 | 42.04651106 | 8.91281E−11 | 3.35861E−09 |
| Hspa8 | 0.2217404 | 12.33062837 | 41.87878413 | 9.71106E−11 | 3.65091E−09 |
| Tmem64 | −0.615622886 | 9.248115386 | 41.48317304 | 1.1889E−10 | 4.45934E−09 |
| Chpt1 | 0.429457135 | 9.179331566 | 41.39841629 | 1.24158E−10 | 4.64615E−09 |
| Ube2c | −0.66752016 | 9.233385993 | 41.22575977 | 1.35623E−10 | 5.06348E−09 |
| BC094916 | 0.462966748 | 9.179306194 | 41.13909389 | 1.41772E−10 | 5.28085E−09 |
| Kmt2e | 0.392340073 | 9.498176274 | 40.93206348 | 1.57614E−10 | 5.85744E−09 |
| Rps13 | 0.126889713 | 11.92521755 | 40.88192763 | 1.6171E−10 | 5.99587E−09 |
| Arhgdib | −0.23489064 | 10.73178038 | 40.82445287 | 1.66536E−10 | 6.16069E−09 |
| Rps11 | −0.128635338 | 12.26306636 | 40.75299459 | 1.72739E−10 | 6.37555E−09 |
| Nrip1 | −0.511508437 | 9.582600354 | 40.74521701 | 1.73427E−10 | 6.3864E−09 |
| Map1lc3b | 0.268218052 | 9.979640755 | 40.65040396 | 1.8205E−10 | 6.68867E−09 |
| Spcs3 | −0.531192179 | 9.347697431 | 40.61133177 | 1.85726E−10 | 6.80829E−09 |
| Cnbp | 0.239051499 | 10.25781376 | 40.46190118 | 2.00488E−10 | 7.33276E−09 |
| Gimap9 | 0.386062289 | 9.581018999 | 40.26864864 | 2.21332E−10 | 8.07689E−09 |
| Siah2 | 0.494945204 | 9.298903622 | 40.20996507 | 2.28082E−10 | 8.30444E−09 |
| Il22 | −1.480069678 | 9.482896849 | 40.13656655 | 2.36814E−10 | 8.60301E−09 |
| Ermn | −0.714912853 | 9.304618523 | 39.83166035 | 2.76821E−10 | 1.00338E−08 |
| Lrrk1 | 0.468677107 | 9.191477397 | 39.74374104 | 2.89567E−10 | 1.04724E−08 |
| Gnb1 | −0.391064061 | 9.703237593 | 39.63283371 | 3.06487E−10 | 1.10595E−08 |
| Gimap5 | 0.370228394 | 9.666351398 | 39.62670839 | 3.0745E−10 | 1.10696E−08 |
| Phlda1 | −0.636144657 | 9.440824176 | 39.56734338 | 3.16939E−10 | 1.13859E−08 |
| Mns1 | −0.550791808 | 9.250778634 | 39.43515476 | 3.39136E−10 | 1.21294E−08 |
| Ugcg | 0.492972956 | 9.205476436 | 39.43590289 | 3.39006E−10 | 1.21294E−08 |
| Gngt2 | −0.558255736 | 9.363452811 | 39.36832194 | 3.50944E−10 | 1.2524E−08 |
| Rpl28 | −0.209984435 | 10.72557562 | 39.30306294 | 3.62872E−10 | 1.29211E−08 |
| Rasd2 | 0.252071753 | 9.160323378 | 39.25591272 | 3.71741E−10 | 1.32078E−08 |
| Cdca3 | −0.432676987 | 9.196735064 | 38.86561366 | 4.54008E−10 | 1.60954E−08 |
| Pyhin1 | 0.507982275 | 9.241830884 | 38.84662598 | 4.58446E−10 | 1.62171E−08 |
| Gpr155 | 0.391019223 | 9.171320995 | 38.72724925 | 4.87358E−10 | 1.71855E−08 |
| Fdps | −0.577186785 | 9.325334883 | 38.7248911 | 4.87947E−10 | 1.71855E−08 |
| Pafah1b1 | 0.325223913 | 9.629847563 | 38.45459246 | 5.60425E−10 | 1.96953E−08 |
| Ahcyl2 | 0.477977983 | 9.277929397 | 38.43033025 | 5.67436E−10 | 1.98984E−08 |
| Tbca | −0.330314256 | 9.754797449 | 38.36938604 | 5.85436E−10 | 2.04852E−08 |
| Sec61g | −0.241152419 | 10.28521272 | 38.33449562 | 5.95997E−10 | 2.08097E−08 |
| Ggt1 | −0.481485267 | 9.203331028 | 38.18802532 | 6.42455E−10 | 2.23835E−08 |
| Stmn1 | −0.969653028 | 9.430288892 | 38.12215366 | 6.64513E−10 | 2.31022E−08 |
| Ms4a4b | 0.250633898 | 10.75182838 | 38.11258677 | 6.6778E−10 | 2.31659E−08 |
| Setbp1 | 0.254687577 | 9.158648722 | 38.06296275 | 6.84981E−10 | 2.37118E−08 |
| Emp1 | −0.541943392 | 9.358546116 | 37.96775029 | 7.19237E−10 | 2.48444E−08 |
| Itpk1 | 0.476912809 | 9.247476152 | 37.77295203 | 7.94761E−10 | 2.73423E−08 |
| Smim3 | −0.541785751 | 9.350048092 | 37.77252938 | 7.94933E−10 | 2.73423E−08 |
| Tap1 | 0.398451698 | 9.443533326 | 37.73515593 | 8.1031E−10 | 2.7812E−08 |
| 2010107E04Rik | −0.300004943 | 9.866343542 | 37.56919881 | 8.82267E−10 | 3.02177E−08 |
| Gimap3 | 0.303898627 | 9.896326654 | 37.49661931 | 9.15716E−10 | 3.1297E−08 |

TABLE 3I
LPL-8 vs. All

| GeneSymbol | logFC | logCPM | LR | PValue | FDR |
|---|---|---|---|---|---|
| 2810417H13Rik | 4.421635993 | 9.33967971 | 7636.915091 | 0 | 0 |
| Hist1h2ap | 2.09574419 | 9.186994176 | 1698.909855 | 0 | 0 |
| Ccnb1 | 2.047353729 | 9.183341398 | 1613.079657 | 0 | 0 |
| Hmgb2 | 2.042066843 | 10.71344588 | 4244.556365 | 0 | 0 |
| Dut | 2.014738394 | 9.371284608 | 1684.500852 | 0 | 0 |
| Tk1 | 1.960291577 | 9.18674202 | 1653.526255 | 0 | 0 |
| Tmpo | 1.849269119 | 9.399807016 | 1521.174414 | 0 | 0 |
| Malat1 | −1.842629645 | 14.66785796 | 1809.716204 | 0 | 0 |
| Alyref | 1.825896572 | 9.471862774 | 1532.732128 | 0 | 0 |
| Tacc3 | 2.134080213 | 9.190377363 | 1956.52509 | 0 | 0 |
| 2700094K13Rik | 1.744910584 | 9.385940771 | 1515.690781 | 0 | 0 |
| Btg1 | −1.646278161 | 12.24806463 | 1559.814146 | 0 | 0 |
| H2afz | 1.610007538 | 10.76844958 | 1970.85919 | 0 | 0 |
| Hmgb1 | 1.500577692 | 10.29770776 | 3186.436388 | 0 | 0 |
| Anp32b | 1.470164134 | 9.653444707 | 1632.790581 | 0 | 0 |
| Rps27 | −1.218652996 | 13.23455517 | 2460.77123 | 0 | 0 |
| Ptma | 1.34946352 | 11.28926493 | 3489.188041 | 0 | 0 |
| Ube2s | 1.317791113 | 10.00342164 | 1614.321852 | 0 | 0 |
| Erh | 1.270303306 | 9.742194835 | 1517.556473 | 0 | 0 |
| Ran | 1.738531804 | 9.802858584 | 3198.770133 | 0 | 0 |
| H2afx | 2.183010877 | 9.352344222 | 1727.503557 | 0 | 0 |
| Stmn1 | 4.321604337 | 9.432950351 | 6902.354621 | 0 | 0 |
| Cdk1 | 2.239500798 | 9.197717135 | 1974.285437 | 0 | 0 |
| Birc5 | 3.697269867 | 9.252643265 | 5794.46549 | 0 | 0 |
| Ube2c | 3.240090144 | 9.234259206 | 2977.10756 | 0 | 0 |
| Rrm2 | 3.204542691 | 9.248571475 | 3515.307592 | 0 | 0 |
| Cks1b | 3.075180267 | 9.262592977 | 4175.962604 | 0 | 0 |
| Nusap1 | 2.820830963 | 9.207702494 | 2838.147599 | 0 | 0 |
| Ccnb2 | 2.788955405 | 9.212862629 | 2442.72995 | 0 | 0 |
| Cdc20 | 2.234954113 | 9.190262317 | 1579.632094 | 0 | 0 |
| Tuba1b | 2.745376143 | 9.732138323 | 4450.514283 | 0 | 0 |
| Asf1b | 2.67891665 | 9.2238456 | 2993.556039 | 0 | 0 |
| Ccna2 | 2.623409165 | 9.194246974 | 3128.32616 | 0 | 0 |
| Cdca8 | 2.623040933 | 9.208417544 | 2645.789881 | 0 | 0 |
| Top2a | 2.621298744 | 9.286093445 | 2205.726859 | 0 | 0 |
| Smc2 | 2.577662882 | 9.241183844 | 2533.553825 | 0 | 0 |
| Hmgn2 | 2.509685643 | 9.446134814 | 2642.35048 | 0 | 0 |
| Ppia | 0.798577016 | 11.56625059 | 1783.957265 | 0 | 0 |
| Spc24 | 2.479141858 | 9.204585668 | 2657.29096 | 0 | 0 |
| Tyms | 2.261136472 | 9.239440332 | 1821.034615 | 0 | 0 |
| Mki67 | 2.362693927 | 9.203436617 | 2323.523705 | 0 | 0 |
| Tubb5 | 2.46042399 | 10.00447555 | 3985.581762 | 0 | 0 |
| Cdca3 | 2.476526339 | 9.197186463 | 2533.838424 | 0 | 0 |
| Gmnn | 1.936716643 | 9.252450911 | 1428.654999 | 0 | 0 |
| Spc25 | 1.802840265 | 9.180565556 | 1423.731434 | 0 | 0 |
| Aurkb | 1.728322959 | 9.176515087 | 1422.512724 | 0 | 0 |
| Rbm3 | 0.993484866 | 10.73203615 | 1418.078619 | 0 | 8.5198E−308 |
| Tpx2 | 1.875133132 | 9.181174219 | 1402.012636 | 7.675E−307 | 2.5849E−304 |
| Fbxo5 | 1.86163473 | 9.18189049 | 1400.863621 | 1.3638E−306 | 4.4995E−304 |
| Rrm1 | 1.877528798 | 9.220046023 | 1360.531885 | 7.9254E−298 | 2.5624E−295 |
| Rpl37a | −0.759463956 | 12.61574203 | 1355.666009 | 9.045E−297 | 2.8671E−294 |
| H2afv | 1.493806059 | 9.565358508 | 1347.992844 | 4.2058E−295 | 1.3075E−292 |
| Ranbp1 | 1.404278127 | 9.627530072 | 1294.507322 | 1.7655E−283 | 5.385E−281 |
| Fau | −0.627429509 | 12.30172216 | 1267.495359 | 1.3092E−277 | 3.9193E−275 |
| Cdkn3 | 1.737691789 | 9.177555326 | 1258.830289 | 1.0002E−275 | 2.9399E−273 |
| Rps14 | −0.606632061 | 12.65499309 | 1255.627678 | 4.9668E−275 | 1.4338E−272 |
| Tipin | 1.929151372 | 9.251510347 | 1251.62808 | 3.6751E−274 | 1.0423E−271 |
| Anp32e | 1.418380455 | 9.517546841 | 1249.901683 | 8.7187E−274 | 2.4301E−271 |
| Rad51 | 1.568013575 | 9.174694718 | 1222.970551 | 6.2115E−268 | 1.702E−265 |
| Ybx1 | 0.833180643 | 10.43974649 | 1218.225603 | 6.6741E−267 | 1.7982E−264 |
| Mcm3 | 1.966718862 | 9.247410025 | 1217.41851 | 9.9952E−267 | 2.6489E−264 |
| Rpl13a | −0.62938187 | 13.13153314 | 1206.09606 | 2.8871E−264 | 7.5278E−262 |
| Cenpe | 1.653275333 | 9.175480245 | 1197.730234 | 1.8993E−262 | 4.8735E−260 |
| Racgap1 | 1.862785236 | 9.232911791 | 1182.243973 | 4.4076E−259 | 1.1133E−256 |
| Kif22 | 1.608511734 | 9.177558442 | 1165.313674 | 2.1072E−255 | 5.2407E−253 |
| Lig1 | 1.894174794 | 9.221722332 | 1152.147372 | 1.5317E−252 | 3.7519E−250 |
| Mcm5 | 1.93647149 | 9.223522441 | 1145.109135 | 5.1862E−251 | 1.2513E−248 |
| Dek | 1.330339959 | 9.608297419 | 1108.987405 | 3.6773E−243 | 8.7423E−241 |
| Lsm5 | 1.16498093 | 9.641405273 | 1067.935311 | 3.0765E−234 | 7.2079E−232 |
| Rps27rt | −1.505651269 | 11.16249609 | 1067.058312 | 4.7717E−234 | 1.102E−231 |
| Pmf1 | 1.64552413 | 9.215533941 | 1064.284361 | 1.9125E−233 | 4.3545E−231 |
| Shcbp1 | 1.506527243 | 9.17318021 | 1057.136768 | 6.8412E−232 | 1.536E−229 |
| Mcm7 | 1.747507535 | 9.230448074 | 1054.889735 | 2.1063E−231 | 4.6646E−229 |
| Cenpf | 1.666917175 | 9.175513788 | 1052.18582 | 8.1514E−231 | 1.7808E−228 |
| Rpl34 | −0.574832472 | 11.98009302 | 1041.129553 | 2.0623E−228 | 4.4453E−226 |
| Ska1 | 1.293432357 | 9.167812846 | 1022.411565 | 2.4146E−224 | 5.1362E−222 |
| Hcst | −1.243433262 | 10.61003451 | 1008.706121 | 2.3008E−221 | 4.8305E−219 |
| Rpl18a | −0.561627543 | 12.84261735 | 1008.01621 | 3.2497E−221 | 6.7351E−219 |
| Gapdh | 0.881759066 | 10.56398779 | 1002.98404 | 4.0331E−220 | 8.2531E−218 |
| Esco2 | 1.327312143 | 9.168283663 | 1000.336843 | 1.5172E−219 | 3.0659E−217 |
| Mad2l1 | 1.648573874 | 9.208311898 | 997.1899147 | 7.3296E−219 | 1.4628E−216 |
| Ezh2 | 1.668172168 | 9.233421923 | 993.4658486 | 4.7267E−218 | 9.3185E−216 |
| Smc4 | 1.616908446 | 9.388203106 | 991.8276439 | 1.0731E−217 | 2.0901E−215 |
| Hint1 | 0.850720336 | 10.08364789 | 978.6553338 | 7.8321E−215 | 1.5073E−212 |
| Hmmr | 1.307226984 | 9.167659346 | 947.1292515 | 5.5818E−208 | 1.0616E−205 |
| Cenpa | 1.819799027 | 9.395189412 | 944.3750433 | 2.2155E−207 | 4.1646E−205 |
| Anapc5 | 1.398347276 | 9.36340878 | 942.2143264 | 6.5338E−207 | 1.2141E−204 |
| Rad51ap1 | 1.283404233 | 9.169013279 | 921.3676391 | 2.2224E−202 | 4.0826E−200 |
| Snrpe | 0.905699046 | 9.828152436 | 913.3257782 | 1.2445E−200 | 2.2604E−198 |
| Plk1 | 1.326982021 | 9.169293368 | 908.8251646 | 1.184E−199 | 2.1267E−197 |
| Uhrf1 | 1.454022969 | 9.180325757 | 906.7397897 | 3.3626E−199 | 5.9736E−197 |
| Nuf2 | 1.191073084 | 9.166477547 | 896.459242 | 5.7748E−197 | 1.0147E−194 |
| Pcna | 1.440069441 | 9.444285329 | 882.2308741 | 7.1558E−194 | 1.2439E−191 |
| Rpl36 | −0.620306689 | 11.87416485 | 879.5581675 | 2.727E−193 | 4.6899E−191 |
| Pfdn5 | −0.85624987 | 10.55314292 | 864.7146686 | 4.5983E−190 | 7.8249E−188 |
| Dbi | 1.368833292 | 9.462823711 | 858.1134292 | 1.2523E−188 | 2.1088E−186 |
| Nap1l1 | 1.185055027 | 9.498411667 | 850.4819 | 5.7122E−187 | 9.5199E−185 |
| Dnajc9 | 1.378564642 | 9.367319875 | 848.3018439 | 1.7012E−186 | 2.8062E−184 |
| Lgals1 | 1.19567371 | 11.62962461 | 845.9087386 | 5.6366E−186 | 9.2042E−184 |
| Pfn1 | 0.80604367 | 12.14562436 | 840.3634126 | 9.0489E−185 | 1.4628E−182 |
| Knstrn | 1.473768485 | 9.18618408 | 839.6230027 | 1.3109E−184 | 2.0982E−182 |
| Cenpm | 1.329309763 | 9.175946219 | 827.6359859 | 5.2921E−182 | 8.3874E−180 |
| Lsm2 | 1.380526709 | 9.294336576 | 823.0375001 | 5.2891E−181 | 8.3013E−179 |
| Cenpw | 1.490656497 | 9.256061112 | 817.7818314 | 7.3455E−180 | 1.1418E−177 |
| Clspn | 1.292669733 | 9.172837657 | 812.9828057 | 8.117E−179 | 1.2497E−176 |
| Pbk | 1.157650531 | 9.166198533 | 812.7880338 | 8.9483E−179 | 1.3647E−176 |
| Hnrnpa3 | 0.835034638 | 10.15657482 | 797.3158283 | 2.0685E−175 | 3.1251E−173 |
| Rps15a | −0.540860736 | 12.40397278 | 794.0829964 | 1.0436E−174 | 1.5621E−172 |
| Ywhah | 1.011739916 | 9.722350699 | 785.426649 | 7.9544E−173 | 1.1797E−170 |
| Prc1 | 1.603165548 | 9.193597027 | 784.9083275 | 1.0311E−172 | 1.5153E−170 |
| Kif11 | 1.323931035 | 9.173024115 | 770.7097078 | 1.2602E−169 | 1.8354E−167 |
| Prdx1 | 0.952910094 | 9.945888311 | 770.5570053 | 1.3603E−169 | 1.9635E−167 |
| Kif20a | 1.187224656 | 9.167869132 | 764.7075308 | 2.5439E−168 | 3.6393E−166 |
| Clic1 | 0.808262932 | 10.32602803 | 762.7488106 | 6.7824E−168 | 9.6179E−166 |
| Hmgb3 | 1.455055923 | 9.208966791 | 761.6651289 | 1.1668E−167 | 1.6403E−165 |
| Mcm6 | 1.533695009 | 9.312060518 | 761.2803647 | 1.4147E−167 | 1.9716E−165 |
| H2-K1 | −0.78477459 | 11.61022938 | 757.7886343 | 8.1261E−167 | 1.1228E−164 |
| Cfl1 | 0.657597691 | 11.11327051 | 745.6811454 | 3.4872E−164 | 4.7775E−162 |
| Casc5 | 1.197590409 | 9.16829785 | 740.8335564 | 3.9494E−163 | 5.3652E−161 |
| Ccdc34 | 1.447741361 | 9.198023808 | 739.5048275 | 7.6816E−163 | 1.0348E−160 |
| Rfc4 | 1.371829427 | 9.206979896 | 738.7492504 | 1.1214E−162 | 1.4982E−160 |
| Ncapg | 1.232809113 | 9.171161894 | 738.4232469 | 1.3202E−162 | 1.7493E−160 |
| Cdca5 | 1.073990128 | 9.165655149 | 727.7176521 | 2.8086E−160 | 3.6913E−158 |
| Rps27l | 1.151450566 | 9.54350515 | 726.2661243 | 5.8091E−160 | 7.5734E−158 |
| Cdc45 | 1.123918696 | 9.168465132 | 721.1692978 | 7.4541E−159 | 9.6403E−157 |
| Slc25a5 | 0.764127042 | 10.39499188 | 716.7972352 | 6.6542E−158 | 8.5375E−156 |
| Incenp | 1.47758554 | 9.217026236 | 712.9734373 | 4.5142E−157 | 5.7462E−155 |
| Nucks1 | 1.286634546 | 9.378701122 | 709.2481525 | 2.9151E−156 | 3.6817E−154 |
| Tcf19 | 1.38178144 | 9.189300183 | 700.8400864 | 1.9635E−154 | 2.4606E−152 |
| Prim1 | 1.368970463 | 9.192585042 | 691.3929603 | 2.2252E−152 | 2.7672E−150 |
| Slbp | 1.125615982 | 9.610102043 | 684.7426234 | 6.2169E−151 | 7.6719E−149 |
| Rpl38 | −0.602301624 | 11.67482758 | 672.1123304 | 3.4693E−148 | 4.2488E−146 |
| Rfc5 | 1.29872706 | 9.203272872 | 669.8028531 | 1.1028E−147 | 1.3404E−145 |
| Depdc1a | 1.080566977 | 9.167299078 | 669.40795 | 1.3439E−147 | 1.6213E−145 |
| Atp5b | 0.684199477 | 10.2144056 | 667.0462036 | 4.385E−147 | 5.251E−145 |
| Actb | 1.341281009 | 13.40284709 | 666.0303963 | 7.2926E−147 | 8.6685E−145 |
| Set | 0.9980865 | 9.773450143 | 658.1483932 | 3.7758E−145 | 4.4555E−143 |
| Fen1 | 1.346059401 | 9.228228642 | 652.6360738 | 5.9679E−144 | 6.991E−142 |
| Rbbp7 | 1.176391939 | 9.398753469 | 651.1468968 | 1.258E−143 | 1.4631E−141 |
| Mcm2 | 1.500969672 | 9.228064884 | 646.9215772 | 1.0438E−142 | 1.2053E−140 |
| Rpl17 | −0.466460143 | 12.29687914 | 646.8673029 | 1.0725E−142 | 1.2297E−140 |
| Mrpl18 | 1.089849217 | 9.412606938 | 646.1342942 | 1.5482E−142 | 1.7626E−140 |
| Hist1h1b | 1.090360538 | 9.168316754 | 640.2582007 | 2.9362E−141 | 3.3193E−139 |
| Fam64a | 0.975294834 | 9.164201841 | 640.020908 | 3.3067E−141 | 3.7122E−139 |
| Sgol1 | 1.052089005 | 9.166666095 | 636.43391 | 1.993E−140 | 2.222E−138 |
| Cdc6 | 0.978089655 | 9.16484216 | 635.7066496 | 2.8687E−140 | 3.1764E−138 |
| Mybl2 | 0.99941864 | 9.165876554 | 633.8085966 | 7.4215E−140 | 8.1616E−138 |
| Shisa5 | −0.780179318 | 11.05001528 | 633.6115951 | 8.191E−140 | 8.947E−138 |
| Ddx39 | 1.198953115 | 9.33417475 | 630.1692678 | 4.5921E−139 | 4.9822E−137 |
| Ndufa4 | 0.938937003 | 9.736118829 | 626.7178333 | 2.5862E−138 | 2.7873E−136 |
| Hnrnpf | 0.638042829 | 10.40327074 | 624.7187992 | 7.0379E−138 | 7.5347E−136 |
| Banf1 | 1.165575172 | 9.354679405 | 619.6666195 | 8.8362E−137 | 9.3978E−135 |
| Neil3 | 0.927084379 | 9.16396466 | 617.4284759 | 2.7105E−136 | 2.864E−134 |
| Ncapd2 | 1.219901417 | 9.183814831 | 616.4156886 | 4.5013E−136 | 4.7252E−134 |
| Cep55 | 1.064886567 | 9.16709682 | 610.2205471 | 1.0018E−134 | 1.0448E−132 |
| Lockd | 1.459848715 | 9.293704619 | 608.8913956 | 1.9493E−134 | 2.02E−132 |
| H2-D1 | −0.53825284 | 12.28212341 | 606.8538604 | 5.4081E−134 | 5.5686E−132 |
| Cit | 1.144554276 | 9.172719925 | 604.7868245 | 1.5228E−133 | 1.558E−131 |
| Nrm | 1.336446732 | 9.221031657 | 603.5070411 | 2.8906E−133 | 2.939E−131 |
| Cbx3 | 1.370284119 | 9.517713266 | 603.3545264 | 3.1201E−133 | 3.1525E−131 |
| Vps37b | −1.31771025 | 10.60451234 | 603.0754448 | 3.5881E−133 | 3.6028E−131 |
| Ckap2l | 0.940613678 | 9.164154324 | 599.4326761 | 2.2243E−132 | 2.2196E−130 |
| Siva1 | 1.268781533 | 9.267321464 | 597.7669302 | 5.1228E−132 | 5.0807E−130 |
| Ddx5 | −0.748471247 | 11.19882055 | 596.8397774 | 8.1503E−132 | 8.034E−130 |
| Cox5b | 0.721979233 | 10.04400742 | 596.6538048 | 8.946E−132 | 8.7649E−130 |
| Dhfr | 1.14149443 | 9.174522367 | 594.1002916 | 3.214E−131 | 3.13E−129 |
| Tagln2 | 0.894454006 | 10.30009936 | 591.5405549 | 1.1583E−130 | 1.1213E−128 |
| Phgdh | 1.224257094 | 9.327085258 | 589.7968759 | 2.7739E−130 | 2.6692E−128 |
| Pnrc1 | −0.970278369 | 10.57662978 | 589.1253447 | 3.8829E−130 | 3.7143E−128 |
| Hnrnpab | 1.039331347 | 9.483542457 | 585.1329548 | 2.8679E−129 | 2.7272E−127 |
| Orc6 | 1.275932538 | 9.234245434 | 582.8772248 | 8.8762E−129 | 8.3914E−127 |
| Rpa3 | 1.251758221 | 9.261572437 | 579.603602 | 4.5741E−128 | 4.2991E−126 |
| Bub1 | 0.865618965 | 9.163406287 | 565.9473494 | 4.2745E−125 | 3.9943E−123 |
| Ywhae | 0.856367486 | 9.677989159 | 564.7314682 | 7.8591E−125 | 7.3017E−123 |
| Eif1 | −0.551908921 | 11.85871741 | 564.4588485 | 9.009E−125 | 8.3223E−123 |
| Atpif1 | 0.97015635 | 9.536110894 | 564.2183893 | 1.0162E−124 | 9.3341E−123 |
| Coro1a | 0.724915443 | 10.77406128 | 561.0456488 | 4.9792E−124 | 4.5476E−122 |
| Chaf1a | 1.055816166 | 9.171628848 | 557.4474793 | 3.0191E−123 | 2.742E−121 |
| Nudt21 | 0.915354374 | 9.525574065 | 556.9359865 | 3.9008E−123 | 3.5229E−121 |
| Usp1 | 1.247336539 | 9.280558501 | 556.4284151 | 5.0299E−123 | 4.5174E−121 |
| Kif2c | 0.832711735 | 9.162579143 | 554.3565171 | 1.42E−122 | 1.2682E−120 |
| Kifc1 | 1.023267071 | 9.168333886 | 552.2123696 | 4.1563E−122 | 3.6918E−120 |
| Melk | 0.932950445 | 9.165498099 | 551.2116811 | 6.8612E−122 | 6.0611E−120 |
| Cenph | 0.913153757 | 9.165027676 | 548.8637447 | 2.2242E−121 | 1.9541E−119 |
| Dlgap5 | 1.093157682 | 9.173768452 | 546.4514966 | 7.4463E−121 | 6.5068E−119 |
| Gins2 | 1.150040727 | 9.177204875 | 542.5356577 | 5.2943E−120 | 4.6015E−118 |
| Rps17 | 0.641426861 | 10.61650637 | 541.2097388 | 1.0286E−119 | 8.8924E−118 |
| Rpl37 | −0.446037144 | 12.07327741 | 536.8593604 | 9.0923E−119 | 7.8184E−117 |
| Tpi1 | 1.264543892 | 9.324340119 | 533.6405003 | 4.5598E−118 | 3.9002E−116 |
| E2f1 | 1.124053463 | 9.176359719 | 532.0684582 | 1.0022E−117 | 8.527E−116 |
| Mxd3 | 0.855249103 | 9.163935023 | 525.4969701 | 2.6954E−116 | 2.2813E−114 |
| Dtymk | 1.209633615 | 9.241120176 | 524.6423187 | 4.1357E−116 | 3.4822E−114 |
| Fignl1 | 1.011624926 | 9.16854214 | 524.1311225 | 5.3428E−116 | 4.4752E−114 |
| Rpl19 | −0.3918592 | 12.11778681 | 523.3530167 | 7.8896E−116 | 6.5744E−114 |
| Snrpd1 | 0.928277472 | 9.48039714 | 521.1514562 | 2.377E−115 | 1.9706E−113 |
| 2700029M09Rik | 1.118173547 | 9.300983612 | 519.4396362 | 5.6035E−115 | 4.6217E−113 |
| Ubb | −0.799096052 | 12.90434358 | 518.3005588 | 9.9147E−115 | 8.1361E−113 |
| Btg2 | −1.147482427 | 10.53608889 | 515.3700463 | 4.3039E−114 | 3.514E−112 |
| Rps16 | −0.424371382 | 12.35159808 | 514.640466 | 6.2028E−114 | 5.039E−112 |
| Ncapg2 | 0.92687554 | 9.167047467 | 512.6513438 | 1.6802E−113 | 1.3581E−111 |
| Trip13 | 0.861097481 | 9.164683183 | 504.775635 | 8.6876E−112 | 6.9872E−110 |
| Hnrnpa2b1 | 0.646022506 | 10.12102555 | 501.1795627 | 5.2641E−111 | 4.2128E−109 |
| Prelid1 | 0.84830737 | 9.694516794 | 499.9674286 | 9.6618E−111 | 7.6942E−109 |
| Mis18bp1 | 0.989123396 | 9.167160156 | 499.953746 | 9.7282E−111 | 7.7092E−109 |
| Kif23 | 1.213665525 | 9.183868072 | 499.4902839 | 1.2271E−110 | 9.6766E−109 |
| Cenpk | 0.977765263 | 9.169824327 | 499.0265271 | 1.548E−110 | 1.2148E−108 |
| Cd53 | −0.807204644 | 10.54707895 | 498.8751605 | 1.67E−110 | 1.3042E−108 |
| Rfc3 | 1.195377368 | 9.204736752 | 498.7666346 | 1.7633E−110 | 1.3705E−108 |
| Nek2 | 0.822261208 | 9.163065177 | 498.6012871 | 1.9156E−110 | 1.4817E−108 |
| Rbm39 | −0.637569471 | 10.67924352 | 496.9893908 | 4.2956E−110 | 3.3068E−108 |
| Rps23 | −0.406286702 | 12.48558878 | 495.1050408 | 1.1041E−109 | 8.4595E−108 |
| Kif4 | 0.767602407 | 9.162351714 | 493.2798993 | 2.7552E−109 | 2.1009E−107 |
| Calm3 | 0.837078543 | 9.648791113 | 492.9071306 | 3.3209E−109 | 2.5205E−107 |
| Mcm4 | 1.237704698 | 9.249504159 | 492.3921324 | 4.2985E−109 | 3.2472E−107 |
| Lrr1 | 0.734760355 | 9.161832843 | 490.4959146 | 1.1115E−108 | 8.3574E−107 |
| Tubg1 | 1.161123937 | 9.198088185 | 489.4483765 | 1.8786E−108 | 1.406E−106 |
| Tcp11l2 | −1.634422416 | 9.433736844 | 488.1472401 | 3.6054E−108 | 2.6859E−106 |
| Aurka | 1.068308243 | 9.172368587 | 484.3164126 | 2.4576E−107 | 1.8225E−105 |
| Ppp1ca | 0.632645325 | 10.2987092 | 481.41766 | 1.0502E−106 | 7.7522E−105 |
| Bub3 | 0.978303874 | 9.426163753 | 481.1590552 | 1.1955E−106 | 8.7845E−105 |
| Gimap6 | −0.955890984 | 10.46579288 | 476.4855883 | 1.243E−105 | 9.0926E−104 |
| Srsf3 | 0.779744858 | 9.795217966 | 474.5266694 | 3.317E−105 | 2.4154E−103 |
| Pkm | 0.696135927 | 10.16306624 | 472.559179 | 8.8895E−105 | 6.4443E−103 |
| Dnmt1 | 1.166056603 | 9.204828421 | 470.7607517 | 2.1889E−104 | 1.5797E−102 |
| Rpl27 | −0.473037067 | 11.47321822 | 470.6997649 | 2.2568E−104 | 1.6215E−102 |
| Rad54l | 0.7727084 | 9.162948533 | 470.3781788 | 2.6514E−104 | 1.8966E−102 |
| Ckap2 | 0.975820995 | 9.166713898 | 468.4327904 | 7.0276E−104 | 5.0048E−102 |
| Cox5a | 0.656023439 | 10.08238974 | 466.6749309 | 1.6956E−103 | 1.2023E−101 |
| Hdgf | 0.972628235 | 9.445842742 | 464.44492 | 5.1833E−103 | 3.6591E−101 |
| Gltp | 0.950180258 | 9.448373052 | 463.8695222 | 6.9155E−103 | 4.8607E−101 |
| Dpy30 | 1.023120178 | 9.328389705 | 461.7599758 | 1.9902E−102 | 1.3928E−100 |
| 1500009L16Rik | 1.477260477 | 9.247571334 | 460.2967524 | 4.143E−102 | 2.8869E−100 |
| Fth1 | −0.680771391 | 11.58497142 | 456.9938832 | 2.1681E−101 | 1.5043E−99 |
| Actg1 | 0.643649634 | 12.10129241 | 456.1278088 | 3.3462E−101 | 2.3118E−99 |
| Mtch1 | 1.10643288 | 9.287149805 | 453.1033746 | 1.5231E−100 | 1.04779E−98 |
| Cbfb | 1.032820636 | 9.370135648 | 452.9002844 | 1.6863E−100 | 1.15512E−98 |
| Bub1b | 0.959966682 | 9.170515641 | 452.1480922 | 2.4583E−100 | 1.67683E−98 |
| Diaph3 | 0.970096611 | 9.171298634 | 449.7795334 | 8.0556E−100 | 5.47168E−98 |
| Lsm3 | 0.997787164 | 9.359624215 | 448.7499742 | 1.3494E−99 | 9.1277E−98 |
| Aunip | 0.670434396 | 9.161326688 | 437.7231237 | 3.38833E−97 | 2.28232E−95 |
| Mif | 0.805872116 | 9.844056396 | 436.030808 | 7.91233E−97 | 5.3075E−95 |
| Rpl23a | −0.403001699 | 12.17600595 | 433.3491727 | 3.03352E−96 | 2.02644E−94 |
| Lbr | 0.95205731 | 9.432005712 | 432.6024105 | 4.4104E−96 | 2.93409E−94 |
| Apitd1 | 0.909142965 | 9.168446716 | 428.856621 | 2.88234E−95 | 1.90967E−93 |
| Hprt | 0.783237884 | 9.584874345 | 428.8234294 | 2.93069E−95 | 1.93377E−93 |
| Gpr132 | −1.004205471 | 9.86576297 | 428.4891485 | 3.46518E−95 | 2.27716E−93 |
| Txn1 | 0.701081293 | 10.00315511 | 427.6708888 | 5.22182E−95 | 3.41765E−93 |
| Dctpp1 | 1.094803772 | 9.308632593 | 425.8304762 | 1.31339E−94 | 8.56142E−93 |
| Lsm6 | 0.903925819 | 9.407177719 | 421.4776028 | 1.16367E−93 | 7.55498E−92 |
| Rnaseh2b | 1.068717641 | 9.200404519 | 421.2669066 | 1.29327E−93 | 8.36282E−92 |
| Gltscr2 | −0.679984369 | 10.29702999 | 420.75614 | 1.67057E−93 | 1.07595E−91 |
| Rabac1 | −0.958867451 | 9.787137327 | 420.4818867 | 1.91672E−93 | 1.22959E−91 |
| Fkbp2 | 1.050724632 | 9.308168075 | 420.4200596 | 1.97704E−93 | 1.26327E−91 |
| Cks2 | 1.338981401 | 9.526677109 | 419.9007985 | 2.56471E−93 | 1.63233E−91 |
| Rps13 | −0.378713374 | 11.92563194 | 414.2002714 | 4.46526E−92 | 2.8308E−90 |
| Ifngr1 | −0.847815544 | 11.1133823 | 413.3409323 | 6.86908E−92 | 4.33772E−90 |
| B2m | −0.355443938 | 12.79997004 | 412.3439791 | 1.13216E−91 | 7.12156E−90 |
| Rps9 | −0.383350015 | 11.99561226 | 412.0841273 | 1.28964E−91 | 8.08074E−90 |
| Cdca2 | 0.821439849 | 9.166105864 | 411.4821296 | 1.74384E−91 | 1.08845E−89 |
| Dtl | 1.106896693 | 9.18924485 | 411.1634528 | 2.04585E−91 | 1.27205E−89 |
| Atp5f1 | 0.643350443 | 9.822922483 | 409.6484511 | 4.37169E−91 | 2.70777E−89 |
| Ptpn18 | −0.758941589 | 10.23424984 | 409.0895315 | 5.78512E−91 | 3.56955E−89 |
| Cdk4 | 0.985774613 | 9.357013312 | 408.7739438 | 6.77655E−91 | 4.16539E−89 |
| Sumo2 | 0.540187794 | 10.26363646 | 405.7481332 | 3.08788E−90 | 1.89086E−88 |
| Tmsb4x | 0.414119715 | 14.2494783 | 405.2692031 | 3.92567E−90 | 2.39481E−88 |
| Ndc80 | 0.962619823 | 9.175406644 | 404.1370231 | 6.92418E−90 | 4.20813E−88 |
| Nme1 | 0.836072856 | 9.654201349 | 402.2147578 | 1.81473E−89 | 1.09876E−87 |
| Ypel3 | −1.161475289 | 9.689654982 | 401.7638557 | 2.27493E−89 | 1.37226E−87 |
| Exosc8 | 0.957856248 | 9.341378608 | 400.5883908 | 4.10062E−89 | 2.46434E−87 |
| Jund | −1.055752186 | 11.83099851 | 400.4895616 | 4.30887E−89 | 2.5799E−87 |
| Metap2 | 0.915717569 | 9.437531426 | 399.7303403 | 6.30429E−89 | 3.76071E−87 |
| Akap13 | −0.793215709 | 10.30793282 | 398.3535435 | 1.25704E−88 | 7.47107E−87 |
| Sdf2l1 | 0.994414931 | 9.406656647 | 396.5991925 | 3.02869E−88 | 1.79348E−86 |
| Sapcd2 | 0.764844708 | 9.163945742 | 395.3862743 | 5.56287E−88 | 3.2821E−86 |
| Lsm4 | 0.755115667 | 9.643812444 | 395.3395144 | 5.6948E−88 | 3.34771E−86 |
| Gm9843 | −0.915259848 | 10.49271816 | 392.3295433 | 2.57476E−87 | 1.5081E−85 |
| Rps21 | −0.62326269 | 10.92456287 | 390.9949889 | 5.02652E−87 | 2.93353E−85 |
| Kif15 | 0.84331882 | 9.168692803 | 390.6634871 | 5.9352E−87 | 3.45139E−85 |
| Ccne1 | 0.880144523 | 9.170524832 | 388.3596153 | 1.88362E−86 | 1.09142E−84 |
| Cbx5 | 1.024894498 | 9.19230852 | 384.5739452 | 1.25648E−85 | 7.2544E−84 |
| Rpl28 | 0.498102261 | 10.72710227 | 383.8744651 | 1.78419E−85 | 1.02645E−83 |
| Hnrnpk | 0.53146514 | 10.15656035 | 383.3645686 | 2.30383E−85 | 1.3207E−83 |
| Psmb2 | 0.681940819 | 9.694383479 | 379.5347498 | 1.57128E−84 | 8.97573E−83 |
| Cmc2 | 1.041743071 | 9.281104329 | 379.2509403 | 1.81152E−84 | 1.03116E−82 |
| Spag5 | 0.706452348 | 9.163496664 | 374.1589094 | 2.3264E−83 | 1.3196E−81 |
| Psat1 | 1.006005575 | 9.197437084 | 372.7504146 | 4.71356E−83 | 2.66432E−81 |
| Poc1a | 0.77141726 | 9.16662515 | 371.2310014 | 1.00964E−82 | 5.68707E−81 |
| Nans | 0.88765801 | 9.365818631 | 368.675622 | 3.63541E−82 | 2.04063E−80 |
| Snrpb | 0.57851806 | 9.840444383 | 365.151612 | 2.12744E−81 | 1.19004E−79 |
| Ppih | 0.980938815 | 9.212614072 | 363.5653328 | 4.71252E−81 | 2.62699E−79 |
| Orai1 | −1.016728243 | 9.884536331 | 362.5648516 | 7.78216E−81 | 4.32325E−79 |
| Hnrnpa1 | 0.775375728 | 9.626739462 | 362.0919788 | 9.86428E−81 | 5.46116E−79 |
| H2-Q7 | −0.697290381 | 10.62132196 | 361.8112175 | 1.13553E−80 | 6.26519E−79 |
| Rpl9 | −0.352804756 | 11.84254424 | 361.6211449 | 1.24907E−80 | 6.86818E−79 |
| Actr3 | 0.58213293 | 10.23457831 | 361.0728975 | 1.64424E−80 | 9.01042E−79 |
| Paics | 1.00190604 | 9.271004075 | 360.0098018 | 2.80188E−80 | 1.53024E−78 |
| Pa2g4 | 0.925734895 | 9.357958 | 359.9198753 | 2.9311E−80 | 1.59543E−78 |
| Rfc2 | 1.00323133 | 9.292032711 | 359.229939 | 4.1425E−80 | 2.24724E−78 |
| Atp5a1 | 0.627339286 | 9.818566382 | 358.8898969 | 4.91255E−80 | 2.65606E−78 |
| Psmd13 | 0.861321347 | 9.383420226 | 358.355887 | 6.42077E−80 | 3.45994E−78 |
| Rpl27a | −0.355187932 | 11.74011835 | 356.7387685 | 1.4445E−79 | 7.75805E−78 |
| Gpsm2 | 0.900753226 | 9.172278225 | 356.3796708 | 1.72946E−79 | 9.25778E−78 |
| Dck | 0.997033102 | 9.191785175 | 356.3011689 | 1.79889E−79 | 9.59766E−78 |
| Ssr2 | 0.86771444 | 9.385650464 | 355.9665601 | 2.12749E−79 | 1.13135E−77 |
| Cmtm7 | 0.934755685 | 9.513680523 | 355.7956012 | 2.3179E−79 | 1.22856E−77 |
| Ssrp1 | 0.866024797 | 9.399511454 | 355.7340231 | 2.39058E−79 | 1.26295E−77 |
| Atp5j | 0.57633364 | 9.979028433 | 355.6056347 | 2.54953E−79 | 1.34253E−77 |
| E2f8 | 0.654183684 | 9.162243958 | 354.7642703 | 3.88751E−79 | 2.04044E−77 |
| Gnai2 | 0.652274503 | 10.17405449 | 353.6515535 | 6.79163E−79 | 3.55318E−77 |
| Lilr4b | −1.271690435 | 9.778014426 | 353.5422247 | 7.17432E−79 | 3.74129E−77 |
| Pkmyt1 | 0.925729349 | 9.188417389 | 353.5314431 | 7.21321E−79 | 3.74948E−77 |
| Slc3a2 | −0.909174492 | 10.08938712 | 352.8096247 | 1.03588E−78 | 5.36734E−77 |
| Raly | 0.620771468 | 9.75257472 | 352.2901969 | 1.34407E−78 | 6.94192E−77 |
| Plk4 | 1.012850484 | 9.204610036 | 351.2839875 | 2.22605E−78 | 1.14606E−76 |
| Ywhaq | 0.759607883 | 9.638348777 | 350.7916279 | 2.84939E−78 | 1.46233E−76 |
| Nhp2 | 0.965616532 | 9.336771458 | 347.9230005 | 1.20071E−77 | 6.14264E−76 |
| Prdx4 | 0.95685906 | 9.185355071 | 347.3384828 | 1.60964E−77 | 8.20866E−76 |
| Ttk | 0.594320893 | 9.161194325 | 346.9284717 | 1.97705E−77 | 1.00506E−75 |
| Ubald2 | −0.910108518 | 10.61959322 | 346.3238081 | 2.67728E−77 | 1.35677E−75 |
| Cox7a2 | 0.563324176 | 9.911912802 | 346.1469211 | 2.9256E−77 | 1.47798E−75 |
| Nup62 | 1.005454222 | 9.206164865 | 346.0910614 | 3.00871E−77 | 1.51523E−75 |
| Cdk2 | 0.981233292 | 9.192856049 | 345.9266664 | 3.26723E−77 | 1.64031E−75 |
| Haus4 | 0.985440832 | 9.208147658 | 345.7031703 | 3.6547E−77 | 1.82916E−75 |
| Hells | 1.087821722 | 9.221371755 | 344.8659781 | 5.56121E−77 | 2.77477E−75 |
| Rps24 | −0.405347353 | 12.00637056 | 344.8094632 | 5.72106E−77 | 2.84574E−75 |
| Tuba1c | 1.190293172 | 9.28147513 | 343.4777414 | 1.11556E−76 | 5.53193E−75 |
| Ect2 | 0.731575748 | 9.165310866 | 343.2551281 | 1.2473E−76 | 6.16632E−75 |
| Chaf1b | 0.954765111 | 9.189533469 | 343.1341221 | 1.32533E−76 | 6.53209E−75 |
| Cotl1 | 0.615218959 | 10.46246128 | 341.9240189 | 2.4314E−76 | 1.19471E−74 |
| Rps26 | 0.445540671 | 10.99491833 | 341.6432091 | 2.79905E−76 | 1.3712E−74 |
| Capzb | 0.553581167 | 10.15938328 | 340.7800705 | 4.31505E−76 | 2.10746E−74 |
| Mrpl42 | 0.937339318 | 9.309229813 | 340.6852723 | 4.52513E−76 | 2.20341E−74 |
| Il2rg | −0.835336928 | 10.13208397 | 338.9763865 | 1.0661E−75 | 5.17553E−74 |
| 4930579G24Rik | 0.94917606 | 9.180623293 | 338.8571651 | 1.13178E−75 | 5.47794E−74 |
| Junb | −0.943261022 | 11.88898263 | 336.5520948 | 3.59562E−75 | 1.73513E−73 |
| Ncaph2 | 0.911927157 | 9.286995177 | 336.4469465 | 3.79031E−75 | 1.82363E−73 |
| Emp3 | 0.793797327 | 9.865128415 | 334.8325828 | 8.51662E−75 | 4.08545E−73 |
| Tnfaip3 | −0.949263973 | 10.12331381 | 334.7605728 | 8.82979E−75 | 4.22315E−73 |
| Gimap3 | −0.902235267 | 9.895535563 | 334.5883792 | 9.62615E−75 | 4.59045E−73 |
| Pgam1 | 0.94467201 | 9.352897068 | 334.0316833 | 1.27262E−74 | 6.05091E−73 |
| Cenpp | 0.638773234 | 9.162492986 | 333.9931832 | 1.29743E−74 | 6.15078E−73 |
| Vim | 0.855008339 | 10.47562831 | 333.5659031 | 1.60746E−74 | 7.59832E−73 |
| Vdac1 | 0.887345388 | 9.320038733 | 332.2835275 | 3.058E−74 | 1.44127E−72 |
| Psma4 | 0.770905394 | 9.544071463 | 331.5369072 | 4.44682E−74 | 2.08975E−72 |
| Cdkn2a | 1.028553278 | 9.187324767 | 331.0716756 | 5.61534E−74 | 2.63123E−72 |
| Ncaph | 0.74820308 | 9.167317127 | 328.5739561 | 1.9651E−73 | 9.18143E−72 |
| Traf1 | −0.842403186 | 10.10488831 | 328.4463672 | 2.09495E−73 | 9.75992E−72 |
| Neurl3 | −1.151353941 | 9.619302411 | 327.1367677 | 4.04028E−73 | 1.87687E−71 |
| Vdac3 | 0.834505977 | 9.365333685 | 325.806207 | 7.87442E−73 | 3.6475E−71 |
| Impdh2 | 0.851176704 | 9.378159405 | 325.6656739 | 8.44944E−73 | 3.90268E−71 |
| Saraf | −0.801725651 | 10.21652778 | 324.5600821 | 1.47109E−72 | 6.77538E−71 |
| Smc1a | 0.940917599 | 9.298422522 | 323.9352535 | 2.01249E−72 | 9.24259E−71 |
| Fdps | 1.173461658 | 9.32579499 | 323.4946271 | 2.5102E−72 | 1.14957E−70 |
| Foxm1 | 0.630168945 | 9.162231863 | 323.3252654 | 2.73274E−72 | 1.24795E−70 |
| Rpl18 | −0.347893772 | 11.80722476 | 320.9543542 | 8.97477E−72 | 4.07545E−70 |
| Pold2 | 0.971125766 | 9.227590909 | 320.9547906 | 8.9728E−72 | 4.07545E−70 |
| Ubc | −0.807279913 | 11.04900464 | 319.2561808 | 2.1034E−71 | 9.52481E−70 |
| 1700097N02Rik | 1.145940071 | 9.227500037 | 317.3504743 | 5.47057E−71 | 2.47031E−69 |
| Rnf26 | 0.918800269 | 9.202614329 | 315.8383443 | 1.16793E−70 | 5.25928E−69 |
| Brip1 | 0.682303488 | 9.163572773 | 315.4570916 | 1.41406E−70 | 6.3499E−69 |
| Cd3g | −0.510882198 | 11.10863423 | 313.5489424 | 3.68235E−70 | 1.649E−68 |
| Anxa2 | 0.921408878 | 9.843831779 | 313.1314335 | 4.54019E−70 | 2.02753E−68 |
| Got1 | −1.503058838 | 9.856934685 | 312.1931746 | 7.26878E−70 | 3.23711E−68 |
| Il7r | −1.159630154 | 9.752616304 | 312.1595397 | 7.39245E−70 | 3.27415E−68 |
| Syce2 | 0.973513782 | 9.186518067 | 312.1627233 | 7.38066E−70 | 3.27415E−68 |
| Ccr2 | −1.363324439 | 9.897377867 | 310.7556679 | 1.49489E−69 | 6.60282E−68 |
| Tubb4b | 0.985482027 | 9.441615817 | 310.1736737 | 2.00166E−69 | 8.81714E−68 |
| Bard1 | 0.7156241 | 9.16703328 | 309.8750016 | 2.32517E−69 | 1.02143E−67 |
| Rps28 | −0.497840868 | 11.70804915 | 309.4766684 | 2.83942E−69 | 1.24396E−67 |
| Zfp367 | 0.842713939 | 9.17481005 | 309.4573584 | 2.86705E−69 | 1.25267E−67 |
| H2-Q4 | −0.898731428 | 9.753196254 | 307.5382342 | 7.50776E−69 | 3.27144E−67 |
| E2f7 | 0.613202567 | 9.162536468 | 307.1197385 | 9.26146E−69 | 4.02475E−67 |
| Hjurp | 0.991807601 | 9.227531733 | 306.0744122 | 1.56461E−68 | 6.78108E−67 |
| Hat1 | 0.94341255 | 9.268309904 | 306.0270499 | 1.60222E−68 | 6.92554E−67 |
| Ndufc2 | 0.713850984 | 9.498050133 | 305.8636729 | 1.73906E−68 | 7.49698E−67 |
| Tpt1 | −0.370821499 | 11.78414713 | 303.762904 | 4.98859E−68 | 2.14483E−66 |

TABLE 3J
LPL-9 vs. All

| GeneSymbol | logFC | logCPM | LR | PValue | FDR |
|---|---|---|---|---|---|
| Vps37b | −3.362309366 | 10.60466024 | 553.5415461 | 2.1358E−122 | 3.4528E−118 |
| Ubald2 | −3.004928151 | 10.62016629 | 523.2616369 | 8.2591E−116 | 6.6758E−112 |
| Junb | −2.255821875 | 11.89138657 | 469.9759664 | 3.2434E−104 | 1.7478E−100 |
| H3f3b | −1.178871544 | 12.34439093 | 430.2412867 | 1.44003E−95 | 5.81989E−92 |
| Ifngr1 | −1.844725191 | 11.11290817 | 395.5179928 | 5.20745E−88 | 1.68367E−84 |
| Bhlhe40 | −2.046123117 | 10.72362795 | 388.2228925 | 2.01724E−86 | 5.43512E−83 |
| AW112010 | 1.174769842 | 11.38326993 | 356.4332675 | 1.68361E−79 | 3.88817E−76 |
| Cd52 | 0.932440577 | 11.79358549 | 322.7676071 | 3.61464E−72 | 7.30427E−69 |
| Tnfrsf4 | −2.757825477 | 10.62218143 | 315.7136553 | 1.24331E−70 | 2.23326E−67 |
| Eif1 | −0.893416996 | 11.85895519 | 311.8836544 | 8.4896E−70 | 1.37243E−66 |
| Odc1 | −2.920870292 | 10.45345646 | 304.1215933 | 4.16711E−68 | 6.12414E−65 |
| Btg1 | −1.28118989 | 12.25019835 | 303.6088436 | 5.38941E−68 | 7.26043E−65 |
| Tmsb4x | 0.581187345 | 14.2491853 | 302.157629 | 1.1161E−67 | 1.38791E−64 |
| Gramd3 | −2.802981311 | 10.20645101 | 301.2956029 | 1.71991E−67 | 1.986E−64 |
| Hspa5 | −1.550719809 | 10.97847279 | 281.551035 | 3.44828E−63 | 3.71632E−60 |
| Ctla4 | −2.513468645 | 10.15301427 | 272.2227397 | 3.71946E−61 | 3.75805E−58 |
| Jund | −1.558244203 | 11.83437558 | 255.6988809 | 1.48623E−57 | 1.41332E−54 |
| Dusp5 | −2.250898553 | 9.994499887 | 254.2297622 | 3.10702E−57 | 2.79045E−54 |
| Pnrc1 | −1.500131777 | 10.57796425 | 251.0077589 | 1.56582E−56 | 1.33226E−53 |
| Ifrd1 | −2.375646739 | 10.02595917 | 249.5366907 | 3.27677E−56 | 2.64861E−53 |
| Prr7 | −2.75505733 | 9.824432052 | 249.3588408 | 3.58277E−56 | 2.75805E−53 |
| Nfkbia | −1.99916409 | 10.83378406 | 242.998769 | 8.72683E−55 | 6.41263E−52 |
| Pim1 | −1.845131276 | 10.82605822 | 235.7608354 | 3.3042E−53 | 2.32242E−50 |
| Tnfaip3 | −1.895396718 | 10.1251933 | 218.6736324 | 1.76077E−49 | 1.18602E−46 |
| Bcl2a1b | −1.652180963 | 10.39213885 | 216.7889935 | 4.53742E−49 | 2.93408E−46 |
| Tgif1 | −2.501012681 | 9.720337198 | 214.475046 | 1.45073E−48 | 9.02017E−46 |
| Icos | −1.845684668 | 10.25638736 | 210.0099268 | 1.36678E−47 | 8.1835E−45 |
| Orai1 | −2.063220862 | 9.884609199 | 204.7883085 | 1.88354E−46 | 1.08748E−43 |
| Il21r | −1.962235376 | 10.19623449 | 203.4843995 | 3.62652E−46 | 2.0216E−43 |
| Crem | −2.638056333 | 10.0668258 | 197.6552503 | 6.78459E−45 | 3.65599E−42 |
| Cd160 | 2.206995649 | 9.33477913 | 182.0716813 | 1.71042E−41 | 8.91955E−39 |
| Cytip | −1.413213899 | 10.18835805 | 178.9512357 | 8.2113E−41 | 4.14824E−38 |
| Btg2 | −1.410335764 | 10.53805397 | 177.5543337 | 1.6574E−40 | 8.11927E−38 |
| Rgs2 | −2.460498875 | 10.05981908 | 172.6649968 | 1.937E−39 | 9.20988E−37 |
| Gna13 | −1.697172725 | 9.985101538 | 171.227445 | 3.99104E−39 | 1.8434E−36 |
| Ramp3 | −3.194515909 | 9.93665422 | 169.8774295 | 7.86932E−39 | 3.53376E−36 |
| Gpr132 | −1.680424877 | 9.866138363 | 162.2711639 | 3.6095E−37 | 1.57706E−34 |
| Rsrp1 | 0.967312622 | 10.002844 | 155.2107284 | 1.25934E−35 | 5.3575E−33 |
| Hspa8 | −0.8840235 | 12.33059378 | 150.107401 | 1.64242E−34 | 6.80805E−32 |
| Kdm6b | −2.430981026 | 9.740743634 | 147.7492684 | 5.38196E−34 | 2.17512E−31 |
| Dnaja1 | −1.426863641 | 10.76378984 | 144.5283367 | 2.7232E−33 | 1.07374E−30 |
| Tbc1d10c | 1.212453764 | 9.522701275 | 142.6216867 | 7.11128E−33 | 2.73717E−30 |
| Traf1 | −1.340039639 | 10.10523686 | 141.5244384 | 1.23557E−32 | 4.64516E−30 |
| Got1 | −2.192776394 | 9.85564432 | 140.3055035 | 2.28248E−32 | 8.38605E−30 |
| Serpinb9 | −2.267244697 | 9.858124549 | 138.1940714 | 6.60916E−32 | 2.3743E−29 |
| Tnfsf8 | −2.583712667 | 10.01779871 | 137.396967 | 9.87356E−32 | 3.46991E−29 |
| Arf4 | −1.368873021 | 9.97476194 | 135.071322 | 3.18519E−31 | 1.09557E−28 |
| Dgat1 | −2.031965751 | 9.778611253 | 134.2108859 | 4.91298E−31 | 1.65465E−28 |
| Fosl2 | −2.219055063 | 9.531543255 | 133.6905648 | 6.38502E−31 | 2.10654E−28 |
| Sub1 | −0.8321836 | 11.1487442 | 132.4885171 | 1.16981E−30 | 3.78224E−28 |
| Lmnb1 | −1.713264533 | 9.781458041 | 132.1686548 | 1.37433E−30 | 4.35636E−28 |
| Nr4a1 | −2.090701396 | 9.630029378 | 131.7109619 | 1.73069E−30 | 5.38046E−28 |
| Pde4b | −1.764328126 | 9.658595001 | 130.8028443 | 2.73461E−30 | 8.34108E−28 |
| Srgn | −0.731632739 | 11.37077253 | 128.7034672 | 7.87464E−30 | 2.35744E−27 |
| Rora | −1.510772327 | 9.982441992 | 126.5708186 | 2.30623E−29 | 6.77865E−27 |
| Nr4a2 | −2.221776331 | 9.521329297 | 124.4447829 | 6.73268E−29 | 1.94358E−26 |
| Zc3h12a | −1.897723673 | 9.536850747 | 123.5261202 | 1.06969E−28 | 3.03379E−26 |
| Ctsw | 0.930564149 | 9.943520924 | 123.4280412 | 1.12389E−28 | 3.13256E−26 |
| Cd48 | 1.086641614 | 9.721154005 | 122.8259103 | 1.52237E−28 | 4.1713E−26 |
| Itm2b | 0.58610183 | 10.97660085 | 122.7037866 | 1.61902E−28 | 4.36218E−26 |
| Cd28 | −1.127738832 | 10.3795945 | 122.4420043 | 1.84737E−28 | 4.89584E−26 |
| Nfkbiz | −1.840698584 | 9.633469169 | 122.3528469 | 1.93228E−28 | 5.03827E−26 |
| Samsn1 | −1.744732813 | 9.795351469 | 121.9802503 | 2.33147E−28 | 5.98263E−26 |
| Itgae | 1.72002363 | 9.275517599 | 121.8280393 | 2.51738E−28 | 6.35875E−26 |
| Rnf125 | −1.773543349 | 9.561296235 | 121.7912041 | 2.56456E−28 | 6.37825E−26 |
| Hif1a | −1.505670523 | 9.774250056 | 119.7969403 | 7.00793E−28 | 1.71652E−25 |
| Slc3a2 | −1.234943945 | 10.08971534 | 118.3977452 | 1.41883E−27 | 3.42341E−25 |
| Cd7 | 1.838925955 | 9.494193586 | 117.2006182 | 2.59451E−27 | 6.16805E−25 |
| Hilpda | −1.952428815 | 9.667484361 | 116.4590431 | 3.77087E−27 | 8.83476E−25 |
| Gpr183 | −1.695171481 | 9.68048749 | 114.4391392 | 1.04422E−26 | 2.41156E−24 |
| Hsp90aa1 | −1.439535496 | 10.76840824 | 113.1243036 | 2.02663E−26 | 4.61443E−24 |
| Ubb | −0.634972292 | 12.9047583 | 113.0579016 | 2.09565E−26 | 4.70531E−24 |
| Ptp4a1 | −1.480320602 | 9.696547191 | 111.6659421 | 4.22885E−26 | 9.36488E−24 |
| Sash3 | 1.316630069 | 9.280817169 | 109.2523432 | 1.42887E−25 | 3.12149E−23 |
| Coro1a | 0.627749664 | 10.77296532 | 106.2309167 | 6.56248E−25 | 1.41452E−22 |
| Actb | 0.907502265 | 13.40512845 | 105.5259815 | 9.36618E−25 | 1.99228E−22 |
| Ddx5 | −0.644534498 | 11.19926646 | 104.0441527 | 1.97857E−24 | 4.15398E−22 |
| Cdkn1a | −1.967048903 | 9.693214366 | 103.6652262 | 2.39559E−24 | 4.96502E−22 |
| Cnn2 | 0.823207984 | 9.902895339 | 100.8167278 | 1.00901E−23 | 2.06476E−21 |
| Lmna | −2.182044064 | 9.713552635 | 99.72532929 | 1.75068E−23 | 3.53768E−21 |
| Arhgap31 | −1.622267935 | 9.6544108 | 96.82563034 | 7.57097E−23 | 1.51102E−20 |
| Xist | −2.484105042 | 9.888219824 | 96.72908307 | 7.9493E−23 | 1.56717E−20 |
| Hspe1 | −1.341804559 | 10.27526821 | 96.08881383 | 1.09844E−22 | 2.13944E−20 |
| Abi3 | 1.306134092 | 9.334710046 | 95.69364832 | 1.3411E−22 | 2.58098E−20 |
| Rrad | −1.921524767 | 9.540636021 | 95.48107352 | 1.49312E−22 | 2.83974E−20 |
| H2afz | −0.974226952 | 10.76535167 | 93.52885095 | 4.00322E−22 | 7.52513E−20 |
| P2ry10 | −1.511318756 | 9.588153587 | 92.98229741 | 5.27642E−22 | 9.80443E−20 |
| Mxd1 | −1.609829978 | 9.600690864 | 92.34661385 | 7.27504E−22 | 1.33646E−19 |
| Bcl2a1d | −1.24869947 | 9.947927963 | 91.96777364 | 8.80995E−22 | 1.60024E−19 |
| Prdx6 | −0.967912422 | 10.1972206 | 91.5415933 | 1.09271E−21 | 1.96275E−19 |
| Lilr4b | −1.440483077 | 9.778164612 | 91.48947472 | 1.12187E−21 | 1.99299E−19 |
| Fam110a | −1.679558179 | 9.494286279 | 90.86306193 | 1.53967E−21 | 2.70547E−19 |
| Hsp90ab1 | −0.858760511 | 11.19689045 | 90.06409284 | 2.30568E−21 | 4.00792E−19 |
| Coq10b | −1.53671868 | 9.509959599 | 88.75880416 | 4.46008E−21 | 7.67039E−19 |
| Cd38 | 1.313844577 | 9.272488233 | 88.58034841 | 4.88114E−21 | 8.30616E−19 |
| Neurl3 | −1.473553701 | 9.619697122 | 88.3936069 | 5.36439E−21 | 9.03341E−19 |
| Fth1 | −0.567457067 | 11.58469958 | 86.84849168 | 1.17161E−20 | 1.95261E−18 |
| Akap13 | −0.848619293 | 10.30887901 | 86.79463258 | 1.20396E−20 | 1.98604E−18 |
| Il4ra | −1.582100662 | 9.499604335 | 86.17634412 | 1.64585E−20 | 2.68755E−18 |
| Satb1 | −1.408753663 | 9.757012703 | 85.69752921 | 2.09675E−20 | 3.3896E−18 |
| Dusp1 | −1.530840947 | 9.939629974 | 85.67286338 | 2.12307E−20 | 3.39817E−18 |
| Srsf5 | −0.903198748 | 10.09111839 | 85.09898727 | 2.83796E−20 | 4.49789E−18 |
| Cd3g | 0.471720583 | 11.10947799 | 84.99935977 | 2.98462E−20 | 4.6844E−18 |
| Ubc | −0.779495031 | 11.05009877 | 84.31851779 | 4.21152E−20 | 6.54648E−18 |
| Rac2 | 0.462122743 | 11.09605968 | 84.20990673 | 4.44936E−20 | 6.85031E−18 |
| Sh2d3c | 1.218788569 | 9.19941797 | 83.84734452 | 5.34493E−20 | 8.15153E−18 |
| Ppp1cc | −0.923264626 | 10.14021217 | 83.69132112 | 5.78386E−20 | 8.73849E−18 |
| Ppp1r15a | −1.499647208 | 9.595479955 | 82.25975518 | 1.19327E−19 | 1.77601E−17 |
| Emb | −1.047852737 | 10.25090288 | 82.25279597 | 1.19748E−19 | 1.77601E−17 |
| Socs3 | −1.700674574 | 9.492344664 | 81.83481982 | 1.47949E−19 | 2.17431E−17 |
| Tnfrsf18 | −1.027239187 | 10.0676873 | 81.19430596 | 2.04581E−19 | 2.9795E−17 |
| Itk | −1.164677422 | 9.814304773 | 81.05650718 | 2.19355E−19 | 3.16615E−17 |
| Gch1 | −1.625035979 | 9.459439724 | 80.85130967 | 2.43357E−19 | 3.48151E−17 |
| Rgcc | −2.013100019 | 9.563703364 | 80.57143357 | 2.80384E−19 | 3.97604E−17 |
| Sp100 | 0.923940711 | 9.553726962 | 80.47733171 | 2.94059E−19 | 4.1337E−17 |
| Herpud1 | −1.409570605 | 9.555337805 | 80.10733411 | 3.54614E−19 | 4.94197E−17 |
| 1810041H14Rik | 1.354825414 | 9.21120969 | 79.71596422 | 4.32294E−19 | 5.97304E−17 |
| Susd3 | 1.085969427 | 9.369960996 | 79.55560648 | 4.68843E−19 | 6.42314E−17 |
| Rinl | 0.974544852 | 9.448193104 | 79.53459661 | 4.73855E−19 | 6.43726E−17 |
| Ube2s | −0.993765017 | 10.00293497 | 78.4765122 | 8.09551E−19 | 1.0906E−16 |
| Dusp10 | −1.717465499 | 9.475523882 | 78.16195183 | 9.49295E−19 | 1.26829E−16 |
| Cd3e | 0.58619296 | 10.374978 | 77.43783083 | 1.36966E−18 | 1.81492E−16 |
| Furin | −1.321937592 | 10.38150283 | 77.13934464 | 1.59312E−18 | 2.07696E−16 |
| Gadd45b | −1.537760322 | 9.727376493 | 77.14809203 | 1.58608E−18 | 2.07696E−16 |
| Rnf157 | −1.556704338 | 9.351515537 | 75.91210474 | 2.96576E−18 | 3.83556E−16 |
| Rel | −1.541336876 | 9.441996052 | 75.09801289 | 4.47917E−18 | 5.74684E−16 |
| Fosb | −1.833251464 | 9.410902867 | 74.90950785 | 4.92791E−18 | 6.2728E−16 |
| Nfkb2 | −1.351023559 | 9.521792478 | 74.24075671 | 6.91478E−18 | 8.73316E−16 |
| Mbnl1 | 0.718281043 | 9.849977707 | 73.3013403 | 1.11292E−17 | 1.39469E−15 |
| Csrnp1 | −1.52260673 | 9.467509493 | 72.98524101 | 1.30623E−17 | 1.62435E−15 |
| Kdm2b | −1.451262627 | 9.446694828 | 72.83509919 | 1.40948E−17 | 1.73936E−15 |
| Vgll4 | −1.268334583 | 9.732088334 | 72.19282088 | 1.95164E−17 | 2.39017E−15 |
| Sqstm1 | −1.19547316 | 9.660274476 | 72.16222124 | 1.98214E−17 | 2.40927E−15 |
| Ptpn7 | 1.086828727 | 9.307714169 | 70.44758814 | 4.72659E−17 | 5.70224E−15 |
| Ptprcap | 0.6964048 | 10.04011854 | 70.09358467 | 5.65566E−17 | 6.77255E−15 |
| Fasl | −1.316167045 | 9.594289128 | 69.62504673 | 7.17204E−17 | 8.52523E−15 |
| Gzma | 1.647822529 | 10.7255561 | 69.16497394 | 9.05622E−17 | 1.06863E−14 |
| Dnajb6 | −0.842670368 | 10.02350611 | 69.03232018 | 9.6863E−17 | 1.1347E−14 |
| Ctsd | 0.815625996 | 9.641722886 | 68.78729408 | 1.09677E−16 | 1.27557E−14 |
| Twf2 | 0.959303046 | 9.389086426 | 68.65428397 | 1.1733E−16 | 1.35482E−14 |
| Dnajb1 | −1.76825082 | 10.3180278 | 68.14703578 | 1.51747E−16 | 1.73982E−14 |
| Arhgdib | 0.495735889 | 10.73174892 | 67.9987325 | 1.63601E−16 | 1.86251E−14 |
| D16Ertd472e | −1.176485153 | 9.623998584 | 67.7530717 | 1.85308E−16 | 2.09489E−14 |
| Maff | −1.470538345 | 9.30528636 | 67.54649177 | 2.05777E−16 | 2.31013E−14 |
| Arid5a | −1.145208968 | 9.656952379 | 67.29570725 | 2.3369E−16 | 2.6054E−14 |
| Bcl11b | 0.918496067 | 9.518633779 | 67.04751294 | 2.65042E−16 | 2.9347E−14 |
| Lcp1 | 0.582999991 | 10.29118475 | 66.78424301 | 3.0291E−16 | 3.33118E−14 |
| Gm12840 | −1.432116469 | 10.16273225 | 65.97236177 | 4.57291E−16 | 4.99497E−14 |
| Ifnar1 | −1.245408007 | 9.536369908 | 65.79959531 | 4.99185E−16 | 5.41599E−14 |
| Ccr9 | 1.552636581 | 9.278717469 | 65.07952837 | 7.19355E−16 | 7.75273E−14 |
| Rbm39 | −0.54358267 | 10.68058111 | 64.55664829 | 9.37965E−16 | 1.00418E−13 |
| Cfl1 | 0.413893348 | 11.1128037 | 64.18560915 | 1.13233E−15 | 1.20429E−13 |
| Cd226 | 1.090319319 | 9.268603322 | 63.99459494 | 1.24761E−15 | 1.31823E−13 |
| Nmrk1 | −1.462700729 | 9.470715907 | 63.44007075 | 1.65321E−15 | 1.73544E−13 |
| Ptp4a2 | −0.807378816 | 9.957611547 | 61.91530565 | 3.58552E−15 | 3.73958E−13 |
| Ppp1r18 | 0.785511357 | 9.553224076 | 60.76888267 | 6.41845E−15 | 6.65132E−13 |
| Clk1 | −0.958741139 | 9.766510063 | 60.56036792 | 7.13563E−15 | 7.34742E−13 |
| Peli1 | 1.000856627 | 9.756264686 | 60.39582424 | 7.75774E−15 | 7.93744E−13 |
| Ndfip1 | −1.004103122 | 9.657936964 | 59.4305111 | 1.2669E−14 | 1.28809E−12 |
| Atp5l | 0.440049168 | 10.47763691 | 59.3617818 | 1.31192E−14 | 1.32553E−12 |
| Tsc22d4 | 0.825276707 | 9.491104749 | 59.08944213 | 1.50665E−14 | 1.51283E−12 |
| H2-Q7 | −0.595713074 | 10.6209645 | 59.05974548 | 1.52957E−14 | 1.52636E−12 |
| Hmgb2 | −0.75986792 | 10.71114671 | 58.85699736 | 1.69558E−14 | 1.68164E−12 |
| Tgif2 | −1.215191779 | 9.272991879 | 58.66690328 | 1.86757E−14 | 1.84092E−12 |
| Ms4a4b | 0.546195203 | 10.75186324 | 58.63792834 | 1.89527E−14 | 1.85691E−12 |
| Dennd4a | −1.326848682 | 9.353141394 | 57.78339494 | 2.9263E−14 | 2.84979E−12 |
| 4930523C07Rik | −0.909932353 | 9.830309453 | 57.35236731 | 3.64325E−14 | 3.52675E−12 |
| Med10 | 0.841244026 | 9.430585613 | 57.27171139 | 3.79576E−14 | 3.65251E−12 |
| Nfkbie | −1.310554368 | 9.398336132 | 56.89961293 | 4.58636E−14 | 4.36616E−12 |
| Gimap4 | 0.496815742 | 10.52507831 | 56.89744873 | 4.59141E−14 | 4.36616E−12 |
| Ier2 | −0.975482231 | 9.828762331 | 56.8735566 | 4.64753E−14 | 4.39369E−12 |
| Relb | −1.186985542 | 9.473479165 | 56.23204924 | 6.44031E−14 | 6.05315E−12 |
| Nkg7 | 0.586133805 | 11.21029847 | 56.16392406 | 6.66737E−14 | 6.23033E−12 |
| Gabpb1 | −1.283624775 | 9.477984036 | 55.60889581 | 8.84226E−14 | 8.21517E−12 |
| Ninj1 | −1.261893918 | 9.366255933 | 54.08307596 | 1.9219E−13 | 1.77539E−11 |
| Tpt1 | −0.325215434 | 11.78327961 | 53.9762298 | 2.0293E−13 | 1.86396E−11 |
| Actn2 | 0.965714565 | 9.563403652 | 53.92498431 | 2.08292E−13 | 1.9024E−11 |
| Lsp1 | 0.535349746 | 10.32145964 | 53.76099128 | 2.26424E−13 | 2.05639E−11 |
| Rnf166 | 0.864754523 | 9.39636208 | 53.53747986 | 2.53706E−13 | 2.29129E−11 |
| Tnfrsf1b | −1.199287429 | 9.452787431 | 53.05790689 | 3.23859E−13 | 2.90861E−11 |
| Ptma | −0.475826165 | 11.28813239 | 52.50547005 | 4.29052E−13 | 3.83207E−11 |
| Lck | 0.468090608 | 10.31669256 | 51.95127581 | 5.68951E−13 | 5.02604E−11 |
| Calm2 | −0.601880603 | 10.41348638 | 51.95191835 | 5.68765E−13 | 5.02604E−11 |
| 3830406C13Rik | 0.90926778 | 9.301772413 | 51.17445011 | 8.45115E−13 | 7.42507E−11 |
| Asb2 | 0.928426346 | 9.480791591 | 50.85879632 | 9.92555E−13 | 8.67332E−11 |
| Hmha1 | 0.652669475 | 9.750300427 | 50.06435076 | 1.48786E−12 | 1.29316E−10 |
| Hspd1 | −1.069494786 | 9.781010215 | 49.88364348 | 1.63139E−12 | 1.41032E−10 |
| Areg | −1.923385968 | 9.386120367 | 49.80905363 | 1.6946E−12 | 1.45718E−10 |
| Apol9b | 0.886455736 | 9.167676629 | 49.50417242 | 1.9795E−12 | 1.69316E−10 |
| Psmb8 | 0.434299989 | 10.43462791 | 49.35929888 | 2.13122E−12 | 1.81333E−10 |
| Dok1 | 0.955105997 | 9.246169552 | 48.3539275 | 3.55831E−12 | 3.01171E−10 |
| Tnfsf11 | −1.406891552 | 9.546989381 | 48.2925552 | 3.67143E−12 | 3.09127E−10 |
| Traf4 | −1.236074371 | 9.35979803 | 48.17646755 | 3.89535E−12 | 3.26281E−10 |
| Lilrb4a | −1.08433259 | 9.64690885 | 47.98172583 | 4.3021E−12 | 3.58494E−10 |
| Ogt | 0.704006931 | 9.590260945 | 47.88727385 | 4.51442E−12 | 3.74257E−10 |
| Cks2 | −1.25825363 | 9.525654391 | 47.68150363 | 5.014E−12 | 4.13553E−10 |
| Sh3bgrl3 | 0.360547616 | 11.2447853 | 47.53739765 | 5.39646E−12 | 4.40602E−10 |
| Abracl | 0.55270054 | 9.935287369 | 47.53825866 | 5.39409E−12 | 4.40602E−10 |
| Tnfrsf9 | −1.42773064 | 9.401976428 | 47.34661806 | 5.94807E−12 | 4.83198E−10 |
| Gm8369 | −0.832266542 | 9.924762802 | 47.22681655 | 6.32294E−12 | 5.11084E−10 |
| Pelo | 0.94676423 | 9.234996852 | 47.03540285 | 6.9716E−12 | 5.60711E−10 |
| Cnbp | −0.563596949 | 10.25781838 | 46.92285364 | 7.38367E−12 | 5.90913E−10 |
| Il2rg | −0.677940365 | 10.13360006 | 46.75705615 | 8.03551E−12 | 6.39912E−10 |
| Ccl5 | 0.919582539 | 13.31121661 | 46.4956193 | 9.18235E−12 | 7.27656E−10 |
| Tob1 | −1.175037157 | 9.420105584 | 46.44658961 | 9.41501E−12 | 7.42454E−10 |
| Nrip1 | −1.112584475 | 9.582654591 | 46.21144382 | 1.06155E−11 | 8.3306E−10 |
| Wdr1 | 0.693500318 | 9.566573023 | 45.91086687 | 1.23758E−11 | 9.66505E−10 |
| Arhgap30 | 0.83146301 | 9.369684229 | 45.82771058 | 1.29124E−11 | 1.00357E−09 |
| Ctsa | 0.775835324 | 9.447143126 | 45.51061925 | 1.51814E−11 | 1.17427E−09 |
| Cd2 | −0.606370172 | 10.31972667 | 45.00764771 | 1.96266E−11 | 1.51088E−09 |
| Sh2d2a | −0.818274264 | 9.846355515 | 44.79805531 | 2.1844E−11 | 1.6736E−09 |
| Cdc42 | 0.401636828 | 10.40470796 | 44.76997118 | 2.21595E−11 | 1.68977E−09 |
| Pfn1 | 0.349264905 | 12.14557694 | 44.61396337 | 2.39974E−11 | 1.82132E−09 |
| Batf | −0.991153728 | 9.653675216 | 44.57301579 | 2.45046E−11 | 1.85113E−09 |
| Ptpn22 | −0.694250299 | 10.09364713 | 44.48055916 | 2.56896E−11 | 1.93162E−09 |
| Nhp2l1 | −0.775628966 | 9.714941638 | 44.38193943 | 2.70168E−11 | 2.02201E−09 |
| Uhrf2 | −1.094311266 | 9.55449696 | 44.32834676 | 2.77666E−11 | 2.06855E−09 |
| Ets1 | −0.570424212 | 10.24999257 | 44.24122854 | 2.90302E−11 | 2.15276E−09 |
| Snx18 | −1.132431204 | 9.331532268 | 43.86391069 | 3.5202E−11 | 2.59852E−09 |
| Mfsd6 | −1.152082823 | 9.369452083 | 43.78578375 | 3.66357E−11 | 2.69205E−09 |
| H2-D1 | −0.274687932 | 12.28188438 | 43.52350373 | 4.18898E−11 | 3.06421E−09 |
| Kpna1 | −1.137981458 | 9.374079051 | 43.41627872 | 4.42492E−11 | 3.22222E−09 |
| Marcksl1 | −1.285775771 | 9.352433315 | 43.24937144 | 4.81892E−11 | 3.4934E−09 |
| Thy1 | 0.455287107 | 10.28931692 | 43.22676676 | 4.87492E−11 | 3.51821E−09 |
| Adam19 | 0.923901938 | 9.250780528 | 43.19470424 | 4.95546E−11 | 3.56044E−09 |
| Socs1 | −1.195444572 | 9.398296399 | 43.16486195 | 5.03162E−11 | 3.59917E−09 |
| Lin54 | −1.157900026 | 9.319410902 | 43.10713832 | 5.18228E−11 | 3.69061E−09 |
| Cd3d | 0.395146119 | 10.55258599 | 42.83010569 | 5.97062E−11 | 4.23338E−09 |
| Dbnl | 0.782581322 | 9.387216148 | 42.72872524 | 6.28821E−11 | 4.43909E−09 |
| Hsph1 | −1.264161017 | 9.472504163 | 42.69129717 | 6.40968E−11 | 4.50517E−09 |
| Gm26917 | 0.710657091 | 9.523381771 | 42.21037434 | 8.19641E−11 | 5.73606E−09 |
| Fam78a | 0.879213037 | 9.208952851 | 42.13614851 | 8.51348E−11 | 5.93228E−09 |
| Dtx1 | 1.008504624 | 9.250253022 | 41.95903475 | 9.32057E−11 | 6.4668E−09 |
| Mxi1 | −1.116673492 | 9.308242179 | 41.93517845 | 9.43498E−11 | 6.5182E−09 |
| Nfkbid | −1.315772495 | 9.347313622 | 41.7580944 | 1.03294E−10 | 7.10573E−09 |
| Klrc1 | −1.210105703 | 9.658447581 | 41.54879294 | 1.14965E−10 | 7.8419E−09 |
| Epsti1 | 0.569607008 | 9.946045371 | 41.54967136 | 1.14914E−10 | 7.8419E−09 |
| Dnajb9 | −1.084099427 | 9.393349046 | 41.13548499 | 1.42034E−10 | 9.64758E−09 |
| Il2rb | −0.831902894 | 9.64634325 | 41.07264289 | 1.46675E−10 | 9.92112E−09 |
| Mob3a | 0.890516107 | 9.226348924 | 40.98635141 | 1.53296E−10 | 1.03258E−08 |
| H2-K1 | −0.346494479 | 11.60996193 | 40.85004492 | 1.64369E−10 | 1.10257E−08 |
| Ifi47 | 0.925508886 | 9.281723133 | 40.83337968 | 1.65777E−10 | 1.10742E−08 |
| Nr4a3 | −1.021483749 | 9.253608924 | 40.74894056 | 1.73097E−10 | 1.15156E−08 |
| Wdr89 | −0.670857344 | 10.42583197 | 40.62234323 | 1.84683E−10 | 1.2236E−08 |
| Cirbp | −0.732767081 | 9.8218496 | 40.59128835 | 1.87641E−10 | 1.23813E−08 |
| Gm8797 | −1.072905955 | 9.417561025 | 40.31410487 | 2.16242E−10 | 1.42104E−08 |
| Ier5 | −0.886024309 | 9.710443697 | 40.1094026 | 2.4013E−10 | 1.5558E−08 |
| Slc38a2 | −1.010291571 | 9.471424004 | 40.10955303 | 2.40112E−10 | 1.5558E−08 |
| Slc25a3 | −0.627950281 | 9.94947131 | 40.12853795 | 2.3779E−10 | 1.5558E−08 |
| Frmd8 | 0.859836984 | 9.275205329 | 40.1056034 | 2.40598E−10 | 1.5558E−08 |
| Eif5 | −0.72406162 | 9.823473225 | 40.07275715 | 2.44678E−10 | 1.57588E−08 |
| Apol9a | 0.608753389 | 9.161172914 | 39.95832528 | 2.59439E−10 | 1.66432E−08 |
| Adora2a | −1.033716215 | 9.426688183 | 39.33091525 | 3.57732E−10 | 2.28581E−08 |
| Cxcl10 | −1.475883536 | 9.358322387 | 39.21145346 | 3.80303E−10 | 2.42046E−08 |
| Arhgef1 | 0.598116191 | 9.623554673 | 39.19793239 | 3.82946E−10 | 2.42772E−08 |
| Gpx4 | −0.613696802 | 10.07608511 | 38.75438539 | 4.80629E−10 | 3.0351E−08 |
| Ncl | −0.572951664 | 10.07277318 | 38.52684203 | 5.40058E−10 | 3.39711E−08 |
| Lamtor4 | 0.793961983 | 9.330433333 | 38.50931949 | 5.44928E−10 | 3.41446E−08 |
| Fus | −0.642585145 | 9.89936241 | 38.25000659 | 6.22369E−10 | 3.88464E−08 |
| Plscr1 | −1.051442773 | 9.521572279 | 38.16059915 | 6.51549E−10 | 4.05113E−08 |
| Tob2 | −1.094490521 | 9.437073308 | 38.07654006 | 6.80231E−10 | 4.21326E−08 |
| Zeb1 | −1.072949092 | 9.369008588 | 37.96671919 | 7.19617E−10 | 4.4402E−08 |
| Vcpkmt | −0.961449373 | 9.468530603 | 37.90218253 | 7.4382E−10 | 4.57209E−08 |

TABLE 3K
LPL-10 vs. All

| GeneSymbol | logFC | logCPM | LR | PValue | FDR |
|---|---|---|---|---|---|
| Rps19 | 1.281201651 | 12.57926737 | 1233.273109 | 3.5827E−270 | 5.7917E−266 |
| Rpl13 | 0.956829763 | 12.63772111 | 932.1398479 | 1.0119E−204 | 8.1792E−201 |
| Rps18 | 0.920425672 | 12.47331244 | 739.6396686 | 7.1801E−163 | 3.8691E−159 |
| Rpl32 | 0.868196367 | 12.44732277 | 680.1448306 | 6.2148E−150 | 2.5117E−146 |
| Rps5 | 0.864582146 | 12.08884438 | 644.630062 | 3.2883E−142 | 1.0632E−138 |
| Rps6 | 0.816445317 | 12.20836226 | 632.6674576 | 1.3142E−139 | 3.541E−136 |
| Rps7 | 0.842852102 | 11.94308668 | 536.7798374 | 9.4618E−119 | 2.1851E−115 |
| Rpl18a | 0.690080812 | 12.84274248 | 470.7611511 | 2.1885E−104 | 4.4223E−101 |
| Rpl14 | 0.747724362 | 11.87702392 | 412.2337163 | 1.19648E−91 | 2.14915E−88 |
| Rplp0 | 0.729699393 | 12.38772174 | 401.1684565 | 3.06604E−89 | 4.95655E−86 |
| Rps24 | 0.773043225 | 12.00582011 | 392.5932715 | 2.25592E−87 | 3.31538E−84 |
| Rps29 | 0.813133225 | 12.24466527 | 380.4817947 | 9.77388E−85 | 1.3167E−81 |
| Rpl23a | 0.692286262 | 12.17553899 | 378.2544662 | 2.98534E−84 | 3.71238E−81 |
| Rps4x | 0.707695218 | 11.84790603 | 373.5198856 | 3.20491E−83 | 3.70075E−80 |
| Rpl17 | 0.660157187 | 12.2966186 | 372.0304773 | 6.76238E−83 | 7.28804E−80 |
| Rpl36a | 0.941421878 | 11.04525672 | 352.1534326 | 1.43947E−78 | 1.45441E−75 |
| Rps23 | 0.623074475 | 12.4852828 | 341.6130126 | 2.84176E−76 | 2.70235E−73 |
| Rps27 | 0.711451235 | 13.23490481 | 297.9212211 | 9.347E−67 | 8.39465E−64 |
| Rpl8 | 0.670651545 | 11.65215182 | 292.7571918 | 1.24681E−65 | 1.06083E−62 |
| Rplp1 | 0.718154214 | 11.70321268 | 282.5577797 | 2.08075E−63 | 1.68187E−60 |
| Rpl12 | 0.895952737 | 10.99231792 | 280.8502016 | 4.90144E−63 | 3.77317E−60 |
| Rps14 | 0.541088503 | 12.65498313 | 275.4661899 | 7.305E−62 | 5.36785E−59 |
| Rps16 | 0.581596764 | 12.35137902 | 275.2382377 | 8.19027E−62 | 5.75669E−59 |
| Rpl35 | 0.847849209 | 11.18855297 | 272.1039925 | 3.94784E−61 | 2.6592E−58 |
| Rps28 | 0.805119029 | 11.70739869 | 270.4060205 | 9.25591E−61 | 5.98524E−58 |
| Rpl21 | 0.642166954 | 11.76870634 | 267.6073941 | 3.7703E−60 | 2.34426E−57 |
| Rps15a | 0.579920698 | 12.40379231 | 264.5275077 | 1.76872E−59 | 1.05901E−56 |
| Rpl39 | 0.66324467 | 11.77723605 | 263.6273811 | 2.77878E−59 | 1.60435E−56 |
| Rps20 | 0.841150732 | 11.06847766 | 256.9273917 | 8.02207E−58 | 4.47189E−55 |
| Rpl9 | 0.584342695 | 11.84222479 | 253.9094837 | 3.64891E−57 | 1.96627E−54 |
| Rpl3 | 0.564967956 | 12.09251912 | 252.5140514 | 7.35131E−57 | 3.83359E−54 |
| Ly6c1 | 1.974532568 | 9.165759038 | 248.5554636 | 5.36247E−56 | 2.70906E−53 |
| Rps3 | 0.53410579 | 11.96702894 | 245.5972451 | 2.36763E−55 | 1.15985E−52 |
| Rpl4 | 0.586800423 | 11.68895541 | 240.4754949 | 3.09761E−54 | 1.47282E−51 |
| Ccr7 | 3.247817647 | 9.273084775 | 237.8112414 | 1.18022E−53 | 5.45125E−51 |
| Rps9 | 0.555129117 | 11.99535989 | 227.9727216 | 1.64993E−51 | 7.40912E−49 |
| Rpl26 | 0.591538476 | 11.57420337 | 217.9166055 | 2.57534E−49 | 1.12522E−46 |
| Rps27a | 0.556559308 | 11.8096941 | 212.0951987 | 4.79472E−48 | 2.03978E−45 |
| Tmsb4x | −0.655091827 | 14.24919003 | 211.3022905 | 7.1408E−48 | 2.95995E−45 |
| Rpl36 | 0.577559407 | 11.87411097 | 210.065659 | 1.32905E−47 | 5.37135E−45 |
| Rplp2 | 0.596131806 | 11.91002396 | 208.3252794 | 3.18603E−47 | 1.25623E−44 |
| S100a11 | −1.433669212 | 10.60258364 | 205.4071252 | 1.38023E−46 | 5.31259E−44 |
| Rpl6 | 0.529407428 | 11.76076159 | 198.9458691 | 3.54707E−45 | 1.33353E−42 |
| Rpl18 | 0.538143421 | 11.80687322 | 196.9817355 | 9.5172E−45 | 3.49671E−42 |
| Rps2 | 0.652273398 | 11.63559296 | 194.8719464 | 2.74764E−44 | 9.87073E−42 |
| Rpl10a | 0.66732504 | 11.14212246 | 193.7961454 | 4.71798E−44 | 1.65806E−41 |
| Rpl35a | 0.546906986 | 11.7233953 | 192.9272701 | 7.30123E−44 | 2.51131E−41 |
| Rps8 | 0.598624386 | 11.59252824 | 191.8021972 | 1.28517E−43 | 4.32834E−41 |
| Rps13 | 0.504165936 | 11.92517909 | 186.704 | 1.66651E−42 | 5.49814E−40 |
| Rpl29 | 0.634590575 | 11.21422123 | 182.0989711 | 1.68711E−41 | 5.45477E−39 |
| Itm2b | −1.023345124 | 10.97660039 | 180.2597916 | 4.25302E−41 | 1.34812E−38 |
| Furin | −2.579368689 | 10.38147822 | 173.642262 | 1.18497E−39 | 3.6839E−37 |
| Rpl23 | 0.549575844 | 11.7085398 | 172.2636291 | 2.37021E−39 | 7.22957E−37 |
| Igfbp4 | 1.65428633 | 9.162779494 | 171.5038314 | 3.47315E−39 | 1.03976E−36 |
| Rpl37 | 0.48788775 | 12.07299733 | 169.5722666 | 9.17463E−39 | 2.69667E−36 |
| Rps12 | 0.770181053 | 10.7705244 | 169.292508 | 1.05607E−38 | 3.04864E−36 |
| Bcl2 | 1.810831323 | 9.436811299 | 163.9128841 | 1.58049E−37 | 4.4825E−35 |
| Rpl37a | 0.48494208 | 12.61560851 | 162.7899384 | 2.78043E−37 | 7.74973E−35 |
| Rpl11 | 0.478674092 | 11.70900459 | 162.7464873 | 2.84187E−37 | 7.78673E−35 |
| Rps15 | 0.534372575 | 11.49069149 | 155.5644172 | 1.05403E−35 | 2.83991E−33 |
| Myl12a | −0.974884236 | 10.67826813 | 148.713865 | 3.31197E−34 | 8.77726E−32 |
| Rpl19 | 0.420544487 | 12.11744557 | 144.6242094 | 2.59489E−33 | 6.76596E−31 |
| Rpl27a | 0.46078461 | 11.73981721 | 144.0432932 | 3.47637E−33 | 8.92046E−31 |
| Cxcr6 | −1.698970736 | 10.38992851 | 137.7218948 | 8.38322E−32 | 2.11755E−29 |
| Rps26 | 0.623715151 | 10.99395953 | 136.8502614 | 1.3003E−31 | 3.23394E−29 |
| Rps3a1 | 0.456678197 | 11.6738534 | 132.4041137 | 1.22062E−30 | 2.98978E−28 |
| S100a10 | −0.999677972 | 10.93991354 | 132.1604121 | 1.38005E−30 | 3.32983E−28 |
| Rpl24 | 0.455232205 | 11.7722356 | 130.7827582 | 2.76242E−30 | 6.56725E−28 |
| Eef1g | 0.710194382 | 10.38511305 | 129.8469723 | 4.42619E−30 | 1.03701E−27 |
| Malat1 | −0.868303233 | 14.66863653 | 121.7575723 | 2.6084E−28 | 6.02392E−26 |
| Ubb | −0.762236847 | 12.90475161 | 121.6648746 | 2.73317E−28 | 6.22316E−26 |
| Klf2 | 1.82007794 | 9.96085156 | 120.599792 | 4.67547E−28 | 1.04977E−25 |
| Lmnb1 | −1.740434723 | 9.781467143 | 118.6227432 | 1.26668E−27 | 2.80509E−25 |
| Rora | −1.598997856 | 9.982434473 | 118.1357505 | 1.61918E−27 | 3.53726E−25 |
| Ly6c2 | 2.908662907 | 9.26599066 | 116.5377425 | 3.62416E−27 | 7.81176E−25 |
| Rgs1 | −1.547472527 | 11.08991268 | 116.1402089 | 4.42854E−27 | 9.41997E−25 |
| Lgals3 | −2.134870543 | 10.20014079 | 115.7963321 | 5.26703E−27 | 1.1058E−24 |
| Rps11 | 0.40051847 | 12.26303456 | 114.4072626 | 1.06115E−26 | 2.19929E−24 |
| Nkg7 | −1.20400333 | 11.21028082 | 113.5760073 | 1.61375E−26 | 3.30227E−24 |
| Actg1 | −0.766856598 | 12.09916335 | 113.4831269 | 1.69114E−26 | 3.41738E−24 |
| Rps21 | 0.618883175 | 10.92367777 | 112.720221 | 2.48475E−26 | 4.95908E−24 |
| Rpl34 | 0.391798461 | 11.98010954 | 112.6674999 | 2.55171E−26 | 5.0306E−24 |
| Hspa5 | −0.993664741 | 10.97848639 | 111.8802176 | 3.79563E−26 | 7.3928E−24 |
| Rpl5 | 0.611265768 | 10.56748783 | 107.8963541 | 2.83208E−25 | 5.4504E−23 |
| Ms4a4c | 1.513771908 | 9.182808698 | 106.6293484 | 5.36724E−25 | 1.02079E−22 |
| AW112010 | −0.988007877 | 11.38325918 | 105.2605754 | 1.07086E−24 | 2.01296E−22 |
| Gm9493 | 0.730569756 | 10.33344069 | 103.3639123 | 2.78909E−24 | 5.18258E−22 |
| Eef1b2 | 0.731249225 | 10.07534778 | 102.783618 | 3.73828E−24 | 6.86739E−22 |
| Satb1 | 1.098437345 | 9.757020312 | 97.00843642 | 6.90326E−23 | 1.25391E−20 |
| Bcl2a1b | −1.109097656 | 10.392145 | 96.86778072 | 7.4115E−23 | 1.33127E−20 |
| H2-D1 | −0.456289267 | 12.28188836 | 94.13284731 | 2.95042E−22 | 5.24138E−20 |
| Rpl15 | 0.533360835 | 10.73579 | 93.09133191 | 4.9936E−22 | 8.77462E−20 |
| Id2 | −1.265718013 | 10.64562852 | 92.85856673 | 5.61682E−22 | 9.73731E−20 |
| Rpsa | 0.43236632 | 11.60003186 | 92.84273602 | 5.66193E−22 | 9.73731E−20 |
| Actn2 | −2.0324523 | 9.563395975 | 91.91238307 | 9.06003E−22 | 1.54173E−19 |
| Hmgb2 | −1.114787643 | 10.71118457 | 91.81149284 | 9.53391E−22 | 1.60547E−19 |
| Sub1 | −0.737071501 | 11.14875127 | 89.22633202 | 3.52131E−21 | 5.8686E−19 |
| Rps10 | 0.438097308 | 11.22174231 | 88.54528471 | 4.96844E−21 | 8.19589E−19 |
| Npm1 | 0.597146323 | 10.46909627 | 86.76885919 | 1.21975E−20 | 1.99177E−18 |
| Prr13 | −0.908211922 | 10.23791505 | 86.28709705 | 1.55621E−20 | 2.51576E−18 |
| Fau | 0.332341064 | 12.3016547 | 85.91336849 | 1.87994E−20 | 3.00901E−18 |
| Rps25 | 0.439065454 | 11.11865132 | 83.92930223 | 5.12788E−20 | 8.12719E−18 |
| H3f3b | −0.530510549 | 12.34438324 | 81.79231815 | 1.51165E−19 | 2.37256E−17 |
| Gm8730 | 0.630359335 | 10.44458017 | 81.7380171 | 1.55376E−19 | 2.4152E−17 |
| Hsp90aa1 | −1.362572556 | 10.76842184 | 81.57324402 | 1.68885E−19 | 2.60019E−17 |
| Rpl38 | 0.406994752 | 11.67453348 | 81.35408494 | 1.88691E−19 | 2.87772E−17 |
| Eef1a1 | 0.337289144 | 12.38172177 | 81.20495197 | 2.03481E−19 | 3.07428E−17 |
| Gzma | −3.37552039 | 10.72559232 | 80.50608545 | 2.89811E−19 | 4.33804E−17 |
| Asb2 | −1.791797421 | 9.480787244 | 79.5436343 | 4.71692E−19 | 6.99576E−17 |
| Rpl13a | 0.303669712 | 13.13151966 | 77.74308062 | 1.17354E−18 | 1.72467E−16 |
| Srgn | −0.611233479 | 11.37079235 | 76.8364188 | 1.85721E−18 | 2.70483E−16 |
| Cd3g | −0.586508979 | 11.10946418 | 76.75229222 | 1.93803E−18 | 2.79734E−16 |
| Glrx | −1.532798827 | 9.565798465 | 76.67665283 | 2.0137E−18 | 2.88084E−16 |
| Ehd1 | −1.565102367 | 9.471456532 | 75.11001307 | 4.45203E−18 | 6.31329E−16 |
| Cldnd1 | −1.473759599 | 9.794845581 | 73.58230211 | 9.65263E−18 | 1.35691E−15 |
| Lef1 | 1.198869799 | 9.289435172 | 73.37527093 | 1.07201E−17 | 1.49397E−15 |
| Lsp1 | −0.866196977 | 10.32146081 | 73.35557179 | 1.08276E−17 | 1.49606E−15 |
| S1pr1 | 1.292942181 | 9.290336532 | 73.11132504 | 1.22539E−17 | 1.67879E−15 |
| Rpl41 | 0.279576202 | 12.79518827 | 72.94991104 | 1.32982E−17 | 1.80655E−15 |
| Lgals1 | −0.901478041 | 11.62752559 | 72.79719209 | 1.43681E−17 | 1.93562E−15 |
| Bcl2a1d | −1.192576834 | 9.947930815 | 72.58098997 | 1.60316E−17 | 2.14187E−15 |
| Gnas | −0.711696987 | 10.37731863 | 72.1045167 | 2.04096E−17 | 2.70444E−15 |
| Dnaja1 | −1.075977432 | 10.76377534 | 70.94687086 | 3.66975E−17 | 4.82319E−15 |
| Cfl1 | −0.562064217 | 11.11281134 | 70.41556668 | 4.80393E−17 | 6.26294E−15 |
| Lat | −0.633486956 | 10.61718655 | 69.93751294 | 6.12132E−17 | 7.91658E−15 |
| Pfn1 | −0.565983596 | 12.1455777 | 69.90284732 | 6.22985E−17 | 7.99299E−15 |
| Dusp10 | 1.197430185 | 9.475524966 | 67.83386892 | 1.77868E−16 | 2.2641E−14 |
| Ppp1ca | −0.731462957 | 10.29792302 | 66.3498649 | 3.77585E−16 | 4.76878E−14 |
| Sidt1 | 1.119553649 | 9.182427708 | 66.2912716 | 3.88977E−16 | 4.87458E−14 |
| Stx11 | −1.479579129 | 9.444380704 | 63.84574775 | 1.34552E−15 | 1.67321E−13 |
| Acot7 | −1.179868494 | 9.577117541 | 63.70742047 | 1.4434E−15 | 1.78122E−13 |
| Gzmb | −2.305535859 | 10.35028864 | 63.25612494 | 1.81502E−15 | 2.22285E−13 |
| Prkar1a | −0.733240671 | 10.10978596 | 61.63694877 | 4.12996E−15 | 5.01991E−13 |
| Hsp90ab1 | −0.785617269 | 11.19690024 | 61.01970556 | 5.65064E−15 | 6.81703E−13 |
| Arpc2 | −0.510019831 | 10.79489599 | 60.85255662 | 6.15136E−15 | 7.36614E−13 |
| Lilr4b | −1.22658672 | 9.778162201 | 60.71605134 | 6.59304E−15 | 7.83699E−13 |
| Atp11b | 1.060573176 | 9.338510173 | 60.4715511 | 7.46496E−15 | 8.80865E−13 |
| Rpl10 | 0.383923631 | 11.48649796 | 60.42271086 | 7.65249E−15 | 8.96451E−13 |
| Rpl13-ps3 | 0.784182986 | 9.661827958 | 60.21552598 | 8.50189E−15 | 9.88788E−13 |
| Ccl5 | −1.592460232 | 13.31119099 | 59.20789454 | 1.41864E−14 | 1.63812E−12 |
| Gzmk | −1.83313636 | 9.363691919 | 58.79982239 | 1.74557E−14 | 2.00134E−12 |
| Fam110a | −1.377632721 | 9.494282763 | 58.37016172 | 2.1716E−14 | 2.47226E−12 |
| mt-Atp6 | 0.342233346 | 12.4713887 | 57.14770681 | 4.04279E−14 | 4.57033E−12 |
| Cdkn1a | −1.510364992 | 9.693205615 | 56.90889505 | 4.56476E−14 | 5.12458E−12 |
| B2m | −0.27669249 | 12.79981273 | 55.08980998 | 1.15146E−13 | 1.28376E−11 |
| Ccr5 | −1.45838306 | 9.514442093 | 55.02519732 | 1.18994E−13 | 1.31758E−11 |
| Cyba | −0.620409355 | 10.43089398 | 54.4652912 | 1.58218E−13 | 1.73996E−11 |
| Shfm1 | −0.655842341 | 10.08130292 | 52.76367247 | 3.76195E−13 | 4.10917E−11 |
| Rpl31 | 0.405514605 | 10.87146125 | 52.0602909 | 5.38224E−13 | 5.83955E−11 |
| D16Ertd472e | −1.069168808 | 9.624003202 | 51.79827152 | 6.15059E−13 | 6.6287E−11 |
| H2-K1 | −0.434001092 | 11.60996642 | 51.75940054 | 6.27357E−13 | 6.71646E−11 |
| Ctla4 | −0.957465278 | 10.15301816 | 51.74174906 | 6.33023E−13 | 6.73253E−11 |
| Odc1 | −1.055185894 | 10.45347895 | 51.72816846 | 6.37417E−13 | 6.73495E−11 |
| Lztfl1 | −1.425717433 | 9.420903385 | 51.65487227 | 6.61662E−13 | 6.94574E−11 |
| Pik3ip1 | 1.016427354 | 9.192060991 | 51.64159498 | 6.66152E−13 | 6.94775E−11 |
| Hspa8 | −0.563966934 | 12.33059948 | 50.45462778 | 1.21952E−12 | 1.26377E−10 |
| Rpl22 | 0.405100818 | 10.6070495 | 49.87592861 | 1.63782E−12 | 1.68643E−10 |
| Ddx5 | −0.472190603 | 11.19925927 | 49.77420376 | 1.72497E−12 | 1.76493E−10 |
| Tsc22d3 | 0.910948391 | 9.704543117 | 49.56953718 | 1.91464E−12 | 1.94667E−10 |
| Gapdh | −0.589658183 | 10.56193028 | 49.41524912 | 2.07129E−12 | 2.09278E−10 |
| Ubl5 | −0.591958802 | 10.27392154 | 49.13961783 | 2.38377E−12 | 2.39354E−10 |
| Rpl23a-ps3 | 0.503304075 | 10.7199609 | 48.2436768 | 3.7641E−12 | 3.7562E−10 |
| Cd6 | −0.869658646 | 9.931712402 | 47.92252282 | 4.43399E−12 | 4.39754E−10 |
| Sh3bgrl3 | −0.457786105 | 11.24479119 | 47.90991163 | 4.4626E−12 | 4.39893E−10 |
| Tceb2 | −0.592952025 | 10.20132116 | 47.42391532 | 5.71808E−12 | 5.60233E−10 |
| Pde2a | 0.987337446 | 9.177524209 | 47.36238186 | 5.90042E−12 | 5.71175E−10 |
| Bhlhe40 | −0.629828205 | 10.72360829 | 47.36525297 | 5.89179E−12 | 5.71175E−10 |
| Maf | −1.20235901 | 9.568711542 | 47.31232275 | 6.05305E−12 | 5.82462E−10 |
| Rpl30 | 0.364473767 | 10.96403143 | 47.01596378 | 7.04109E−12 | 6.73528E−10 |
| S100a4 | −1.45718699 | 9.866407172 | 46.84257616 | 7.69239E−12 | 7.31501E−10 |
| Abracl | −0.751666052 | 9.935290448 | 46.8085049 | 7.8273E−12 | 7.39977E−10 |
| Gnb2l1 | 0.413063836 | 10.54000009 | 46.77545408 | 7.96042E−12 | 7.48187E−10 |
| Ifng | −1.860168053 | 9.686682514 | 46.5515956 | 8.92376E−12 | 8.33881E−10 |
| Pkp3 | −0.992944373 | 9.709717317 | 45.7083226 | 1.37238E−11 | 1.27506E−09 |
| Smim3 | −1.264653022 | 9.350059212 | 45.17112102 | 1.80548E−11 | 1.66785E−09 |
| Gdi2 | −0.736720685 | 9.887611627 | 44.73070501 | 2.26084E−11 | 2.07663E−09 |
| Oaz1 | −0.345126362 | 11.23125359 | 44.4427732 | 2.61902E−11 | 2.39204E−09 |
| Il7r | 0.762433294 | 9.753528328 | 44.19134887 | 2.97794E−11 | 2.70458E−09 |
| Manf | −0.873653643 | 9.719335527 | 44.10873804 | 3.10631E−11 | 2.8054E−09 |
| Lpxn | −0.856247558 | 9.719586419 | 43.85349118 | 3.53899E−11 | 3.17841E−09 |
| Dynll1 | −0.677834564 | 10.05622605 | 43.12939789 | 5.12366E−11 | 4.57619E−09 |
| Dph5 | 0.964698786 | 9.213978081 | 43.09901238 | 5.20385E−11 | 4.62228E−09 |
| Cd82 | −0.685765836 | 10.1275368 | 42.58772697 | 6.75821E−11 | 5.97012E−09 |
| Cd74 | −1.790229737 | 9.434235572 | 42.16081243 | 8.40678E−11 | 7.38609E−09 |
| Hcls1 | −0.884940162 | 9.587943614 | 41.89048756 | 9.65311E−11 | 8.43525E−09 |
| Arl6ip1 | −0.744621815 | 9.94432139 | 41.67387503 | 1.0784E−10 | 9.37284E−09 |
| Sh3bp5 | 0.906477381 | 9.188376986 | 41.65015897 | 1.09157E−10 | 9.43649E−09 |
| Tank | −1.122428469 | 9.370123349 | 41.51736594 | 1.16829E−10 | 1.0046E−08 |
| Tmem176a | −1.561868972 | 9.404491611 | 41.45307093 | 1.20735E−10 | 1.0327E−08 |
| Ermn | −1.384380136 | 9.30459238 | 41.37987416 | 1.25341E−10 | 1.06646E−08 |
| Ikzf3 | −1.234027314 | 9.310526312 | 41.26331133 | 1.33043E−10 | 1.12606E−08 |
| Clic1 | −0.576996189 | 10.32552274 | 41.07895162 | 1.46202E−10 | 1.23099E−08 |
| Gm8369 | 0.656116673 | 9.924740414 | 40.88479166 | 1.61473E−10 | 1.35252E−08 |
| Uap1 | −1.127448986 | 9.373339988 | 40.5920089 | 1.87572E−10 | 1.56304E−08 |
| Ubald2 | −0.647705139 | 10.62015695 | 40.48582272 | 1.98048E−10 | 1.64187E−08 |
| Cd5 | −0.942120559 | 9.629220038 | 40.26054766 | 2.22252E−10 | 1.83313E−08 |
| Nr3c1 | −1.154618397 | 9.448746596 | 40.15577128 | 2.34498E−10 | 1.92431E−08 |
| Anxa2 | −0.939195153 | 9.842340624 | 40.03380626 | 2.49605E−10 | 2.03794E−08 |
| Tmem176b | −1.478158733 | 9.44810635 | 39.8238096 | 2.77936E−10 | 2.25785E−08 |
| Ikzf2 | −1.348475271 | 9.298837321 | 39.71435404 | 2.93957E−10 | 2.36423E−08 |
| AB124611 | 0.925207051 | 9.180451135 | 39.72365158 | 2.92561E−10 | 2.36423E−08 |
| Serpinb1a | −1.843570985 | 9.512641434 | 39.59191023 | 3.12977E−10 | 2.50475E−08 |
| Stk24 | −0.91383667 | 9.608549705 | 39.52447346 | 3.23974E−10 | 2.57998E−08 |
| Efhd2 | −0.999146886 | 9.465521733 | 39.13275436 | 3.95945E−10 | 3.13767E−08 |
| Ucp2 | −0.65915216 | 10.0128664 | 39.02218127 | 4.19018E−10 | 3.30431E−08 |
| Calr | −0.677910097 | 10.0207225 | 38.16235309 | 6.50963E−10 | 5.10848E−08 |
| Cdc42 | −0.468486577 | 10.40471662 | 38.14980497 | 6.55163E−10 | 5.1166E−08 |
| Fam101b | 0.953455397 | 9.174681163 | 38.04046828 | 6.92924E−10 | 5.38549E−08 |
| Serpina3g | −1.177709452 | 9.674291957 | 38.02477696 | 6.98519E−10 | 5.403E−08 |
| Socs3 | 0.876228257 | 9.49233387 | 37.92020104 | 7.36982E−10 | 5.67336E−08 |
| Hsp90b1 | −0.822947909 | 9.703951568 | 37.82898625 | 7.72258E−10 | 5.91674E−08 |

TABLE 3L
LPL-11 vs. All

| GeneSymbol | logFC | logCPM | LR | PValue | FDR |
|---|---|---|---|---|---|
| Gata3 | 2.78725309 | 9.445536089 | 651.0499191 | 1.3206E−143 | 2.1349E−139 |
| Ccl5 | −5.30741133 | 13.31117347 | 348.0391179 | 1.1328E−77 | 9.15641E−74 |
| Tnfsf8 | 2.436704618 | 10.01782193 | 323.7980169 | 2.15589E−72 | 1.16174E−68 |
| Rpl37a | 0.622775272 | 12.61561004 | 294.538539 | 5.10124E−66 | 2.06167E−62 |
| Il5 | 1.913428082 | 9.162955407 | 286.2336147 | 3.2903E−64 | 1.06382E−60 |
| Socs2 | 2.183612465 | 9.351639381 | 270.9835186 | 6.92719E−61 | 1.86642E−57 |
| Cldnd1 | 1.666913086 | 9.794858969 | 252.1526177 | 8.81368E−57 | 2.03546E−53 |
| Gpx4 | 1.087119142 | 10.07609679 | 226.2535433 | 3.91211E−51 | 7.48478E−48 |
| Sub1 | 0.883083724 | 11.1487561 | 226.1278761 | 4.16696E−51 | 7.48478E−48 |
| AW112010 | −1.474129989 | 11.38325184 | 209.9972312 | 1.37553E−47 | 2.22368E−44 |
| Ms4a4b | −1.446723816 | 10.75185359 | 195.3518584 | 2.15883E−44 | 3.1727E−41 |
| Ramp3 | 2.145977545 | 9.936713117 | 192.8546395 | 7.57266E−44 | 1.02016E−40 |
| Il4 | 2.011817772 | 9.174329336 | 191.3077471 | 1.64772E−43 | 2.049E−40 |
| Itk | 1.193676475 | 9.814313226 | 190.8271332 | 2.09791E−43 | 2.42249E−40 |
| Samsn1 | 1.384203515 | 9.795369332 | 186.5931584 | 1.762E−42 | 1.89896E−39 |
| Pfn1 | −0.962844016 | 12.14557821 | 185.1929867 | 3.56178E−42 | 3.59874E−39 |
| Hilpda | 1.535712444 | 9.667495774 | 172.5446413 | 2.05786E−39 | 1.9569E−36 |
| Nkg7 | −1.499609631 | 11.21026875 | 166.4588249 | 4.39179E−38 | 3.94431E−35 |
| Lgals7 | 2.061472613 | 9.201968358 | 164.5758087 | 1.13233E−37 | 9.63437E−35 |
| Il1rl1 | 1.727815818 | 9.173799372 | 163.5329974 | 1.91329E−37 | 1.54652E−34 |
| H2afz | 1.030291278 | 10.7654377 | 151.5333042 | 8.01359E−35 | 6.16893E−32 |
| Tnfrsf18 | 1.025507117 | 10.06770513 | 150.5249306 | 1.33114E−34 | 9.78146E−32 |
| Rps9 | 0.436769402 | 11.99536279 | 149.9878754 | 1.74425E−34 | 1.22598E−31 |
| Rpl23 | 0.492111858 | 11.70855206 | 147.5273126 | 6.0181E−34 | 4.05369E−31 |
| Ptpn13 | 1.508179564 | 9.18673375 | 145.8278672 | 1.4157E−33 | 9.1545E−31 |
| Fam213a | 1.52409344 | 9.188402088 | 144.3827407 | 2.93029E−33 | 1.82197E−30 |
| Fam46a | 1.816227914 | 9.5043122 | 140.7033004 | 1.86819E−32 | 1.11856E−29 |
| Actb | −1.645143094 | 13.40506597 | 136.9264382 | 1.25136E−31 | 7.22482E−29 |
| Ms4a6b | −1.276285498 | 10.19495673 | 136.8462663 | 1.30292E−31 | 7.26309E−29 |
| Dusp10 | 1.527437104 | 9.475525704 | 135.6542382 | 2.37485E−31 | 1.27972E−28 |
| Crem | 1.428290135 | 10.06687059 | 135.0047119 | 3.29386E−31 | 1.7177E−28 |
| Gm8369 | −1.539746047 | 9.92472512 | 126.5999821 | 2.27259E−29 | 1.14808E−26 |
| Ptgir | 1.113446316 | 9.163290516 | 126.4785278 | 2.41602E−29 | 1.18356E−26 |
| Rpl21 | 0.435636427 | 11.76870978 | 125.261045 | 4.46212E−29 | 2.12161E−26 |
| Rps28 | 0.55761229 | 11.70740612 | 123.962248 | 8.58618E−29 | 3.96583E−26 |
| Tpt1 | 0.441970525 | 11.78330363 | 122.7748436 | 1.56206E−28 | 7.01453E−26 |
| Got1 | 1.376458958 | 9.855680992 | 121.8512873 | 2.48806E−28 | 1.08708E−25 |
| Calca | 3.14777933 | 9.213255376 | 121.6092469 | 2.81089E−28 | 1.19581E−25 |
| Eef1a1 | 0.393660221 | 12.38173251 | 121.482608 | 2.99616E−28 | 1.24195E−25 |
| Spcs3 | 1.235400868 | 9.34767364 | 119.8851623 | 6.70309E−28 | 2.70905E−25 |
| Tnfrsf4 | 1.148431166 | 10.62221384 | 117.9370243 | 1.78981E−27 | 7.05711E−25 |
| 9230102004Rik | 0.982375521 | 9.161433502 | 116.704018 | 3.3327E−27 | 1.28277E−24 |
| Rps12-ps3 | 0.943779186 | 9.914308593 | 115.0032374 | 7.85691E−27 | 2.95383E−24 |
| Frmd4b | 1.36706003 | 9.262065137 | 114.9482961 | 8.07763E−27 | 2.9678E−24 |
| Rps12 | 0.623247383 | 10.77054436 | 114.1870765 | 1.18577E−26 | 4.2598E−24 |
| Wdr89 | 0.942765197 | 10.42589899 | 111.5161523 | 4.56074E−26 | 1.6028E−23 |
| Rplp2 | 0.431571166 | 11.91003287 | 109.5583778 | 1.22445E−25 | 4.21158E−23 |
| Arpc3 | 0.640798492 | 10.54004275 | 108.871016 | 1.73198E−25 | 5.83315E−23 |
| Serpina3g | −2.073186267 | 9.674281316 | 107.2026936 | 4.0189E−25 | 1.32591E−22 |
| Rps21 | 0.584104575 | 10.92368799 | 107.0529425 | 4.33434E−25 | 1.40138E−22 |
| Rpl23a | 0.377714752 | 12.17554432 | 106.7452237 | 5.06241E−25 | 1.60469E−22 |
| Lcp1 | −0.975234565 | 10.29117732 | 105.6943068 | 8.60342E−25 | 2.67467E−22 |
| Rps29 | 0.448556786 | 12.24466765 | 104.4778208 | 1.58963E−24 | 4.84867E−22 |
| Epsti1 | −1.239773795 | 9.946039862 | 104.081923 | 1.94121E−24 | 5.81142E−22 |
| Serinc3 | 1.130652275 | 9.437689744 | 103.9755135 | 2.04832E−24 | 5.91306E−22 |
| Bcl2a1b | 0.824160134 | 10.3921492 | 103.9953976 | 2.02787E−24 | 5.91306E−22 |
| Rps24 | 0.413547684 | 12.0058264 | 103.6828838 | 2.37434E−24 | 6.73395E−22 |
| Lsp1 | −0.985266793 | 10.32146161 | 103.541068 | 2.55052E−24 | 7.10892E−22 |
| Tnfaip8 | 1.025889715 | 9.63021053 | 102.1887411 | 5.0476E−24 | 1.38304E−21 |
| Arhgdib | −0.776390788 | 10.73175766 | 101.3554603 | 7.6873E−24 | 2.07122E−21 |
| Gimap4 | −0.848082586 | 10.52505641 | 100.3208288 | 1.29607E−23 | 3.43479E−21 |
| Rps20 | 0.532114639 | 11.06849484 | 100.0530592 | 1.48369E−23 | 3.86859E−21 |
| Rpl35a | 0.383212685 | 11.72340173 | 98.87799881 | 2.68546E−23 | 6.89098E−21 |
| Rps7 | 0.381427202 | 11.94309364 | 98.49929886 | 3.2514E−23 | 8.21283E−21 |
| Rpl26 | 0.386577119 | 11.57421057 | 96.7395076 | 7.90755E−23 | 1.96667E−20 |
| Ltb | −1.399434777 | 10.15519086 | 96.12700077 | 1.07745E−22 | 2.63911E−20 |
| Rpl30 | 0.473159497 | 10.96404079 | 94.5744842 | 2.36041E−22 | 5.69529E−20 |
| Gramd3 | −1.361397858 | 10.20644171 | 91.6866376 | 1.01548E−21 | 2.41416E−19 |
| Dgat1 | 1.136334074 | 9.778640319 | 91.23289682 | 1.27719E−21 | 2.99233E−19 |
| Gzma | −3.702496857 | 10.72561706 | 90.72545605 | 1.65056E−21 | 3.81185E−19 |
| Epas1 | 1.267743009 | 9.190648446 | 90.47197387 | 1.87616E−21 | 4.27182E−19 |
| Fdx1 | 1.231395993 | 9.293641737 | 90.38007272 | 1.96536E−21 | 4.41277E−19 |
| Cd48 | −1.406318811 | 9.721163919 | 90.22654102 | 2.12394E−21 | 4.7035E−19 |
| Rps23 | 0.329029763 | 12.48528629 | 90.09886224 | 2.26552E−21 | 4.94924E−19 |
| Rps15 | 0.390713377 | 11.49069805 | 87.8944932 | 6.90406E−21 | 1.48815E−18 |
| Il17rb | 0.719078081 | 9.159374823 | 85.61480456 | 2.18632E−20 | 4.65054E−18 |
| Rpl9-ps6 | 0.874151789 | 9.840821559 | 85.0333623 | 2.93373E−20 | 6.15931E−18 |
| Gpr18 | −1.78216716 | 9.406620641 | 84.12800323 | 4.63755E−20 | 9.61162E−18 |
| Ipmk | 1.273590944 | 9.271358545 | 83.86447633 | 5.29882E−20 | 1.08431E−17 |
| Coro1a | −0.73247519 | 10.77299576 | 83.22874718 | 7.30871E−20 | 1.47691E−17 |
| Abhd17c | 1.101899836 | 9.339495287 | 82.97818141 | 8.29641E−20 | 1.6558E−17 |
| Rpl9 | 0.330253784 | 11.84222846 | 82.11044546 | 1.28691E−19 | 2.53709E−17 |
| Rps10 | 0.393627927 | 11.22175565 | 80.59909901 | 2.76486E−19 | 5.38514E−17 |
| Gzmb | −2.701378123 | 10.35029484 | 80.36660647 | 3.11007E−19 | 5.9854E−17 |
| Cd3e | −0.76840368 | 10.37494886 | 80.21369816 | 3.3603E−19 | 6.39089E−17 |
| Gm9843 | 0.686156797 | 10.49140966 | 78.62252705 | 7.51871E−19 | 1.41334E−16 |
| Ccr5 | −1.657767592 | 9.514438228 | 78.43448041 | 8.26961E−19 | 1.53663E−16 |
| Cfl1 | −0.559969156 | 11.11281656 | 78.41189297 | 8.36471E−19 | 1.53663E−16 |
| Rpl17 | 0.311214497 | 12.29662158 | 77.9954965 | 1.03276E−18 | 1.87591E−16 |
| Il13 | 1.584467555 | 9.164846146 | 76.40414181 | 2.31166E−18 | 4.15225E−16 |
| Rac2 | −0.536378869 | 11.09605583 | 75.73073771 | 3.25107E−18 | 5.77547E−16 |
| Rpl36a | 0.451614988 | 11.04526124 | 73.03434504 | 1.27413E−17 | 2.23887E−15 |
| Rps3a1 | 0.326717425 | 11.67386184 | 72.76462129 | 1.46072E−17 | 2.53914E−15 |
| Rpl38 | 0.370765232 | 11.67453685 | 72.51121348 | 1.66086E−17 | 2.85632E−15 |
| Rps27rt | 0.626079443 | 11.16192697 | 72.33582672 | 1.81522E−17 | 3.08893E−15 |
| Rpl6l | 0.60326212 | 10.20612343 | 72.15571984 | 1.98868E−17 | 3.34886E−15 |
| Fosl2 | 1.091970828 | 9.531552388 | 71.34388687 | 3.00086E−17 | 5.00123E−15 |
| Vps37b | 0.724244734 | 10.60465641 | 71.30913819 | 3.05418E−17 | 5.01342E−15 |
| Dnajc15 | −0.897639796 | 9.893114381 | 71.29881659 | 3.0702E−17 | 5.01342E−15 |
| Cd7 | −2.214869992 | 9.494180616 | 70.61890677 | 4.33344E−17 | 7.00544E−15 |
| Klf2 | −2.182862403 | 9.960838309 | 70.52757429 | 4.53878E−17 | 7.26474E−15 |
| Rpl23a-ps3 | 0.582425672 | 10.71997612 | 70.42054103 | 4.79184E−17 | 7.59459E−15 |
| Rpl13 | 0.292940011 | 12.63772195 | 69.15470278 | 9.1035E−17 | 1.42881E−14 |
| Rpl6 | 0.305809539 | 11.76076847 | 69.07620569 | 9.47314E−17 | 1.47253E−14 |
| Ell2 | 1.083692654 | 9.233842501 | 68.53144299 | 1.24871E−16 | 1.92253E−14 |
| Cd200r1 | 1.056706352 | 9.188142445 | 68.15502474 | 1.51134E−16 | 2.28339E−14 |
| Ctla2a | 1.029049134 | 10.33550568 | 68.16832127 | 1.50118E−16 | 2.28339E−14 |
| Nfkb1 | 0.863972112 | 9.53708087 | 68.04758287 | 1.59597E−16 | 2.38894E−14 |
| Isy1 | 0.955123258 | 9.388775206 | 68.01603383 | 1.62171E−16 | 2.4052E−14 |
| Cxcr3 | −1.31367325 | 9.55137858 | 67.18697187 | 2.46941E−16 | 3.62913E−14 |
| Abracl | −0.827023382 | 9.935292552 | 66.38855383 | 3.70246E−16 | 5.39225E−14 |
| Elovl5 | 1.000670868 | 9.322047714 | 66.29789289 | 3.87673E−16 | 5.59564E−14 |
| Rplp1 | 0.354849578 | 11.70322227 | 64.41465545 | 1.00806E−15 | 1.44214E−13 |
| Ubac2 | −1.232880489 | 9.42377083 | 64.35941478 | 1.03672E−15 | 1.47014E−13 |
| Cyba | −0.621006408 | 10.43089692 | 64.18614099 | 1.13202E−15 | 1.59133E−13 |
| Rpl10-ps3 | 0.822487389 | 9.578827067 | 64.11901202 | 1.17126E−15 | 1.63229E−13 |
| Amica1 | −1.523510324 | 9.335288888 | 63.701196 | 1.44797E−15 | 2.00067E−13 |
| Rpl12 | 0.445699316 | 10.99232629 | 63.59632141 | 1.52714E−15 | 2.09218E−13 |
| Emb | 0.74683201 | 10.25092353 | 63.34678603 | 1.73338E−15 | 2.35477E−13 |
| Rps6 | 0.277808542 | 12.20836688 | 63.27756685 | 1.79537E−15 | 2.41867E−13 |
| Rpl19 | 0.270133762 | 12.11744948 | 63.03407253 | 2.03162E−15 | 2.71431E−13 |
| Rpa2 | −1.380531757 | 9.392692325 | 63.01445595 | 2.05195E−15 | 2.719E−13 |
| Fau | 0.274449368 | 12.3016555 | 62.71392745 | 2.39019E−15 | 3.14145E−13 |
| Gnai2 | −0.748009519 | 10.17430832 | 62.26042219 | 3.00912E−15 | 3.83955E−13 |
| Rps27a | 0.301110145 | 11.80970228 | 62.2783555 | 2.98184E−15 | 3.83955E−13 |
| Elf1 | 0.782440901 | 9.573205604 | 62.2557001 | 3.01635E−15 | 3.83955E−13 |
| Btg1 | −0.611493 | 12.25013986 | 62.29398453 | 2.95827E−15 | 3.83955E−13 |
| Limd2 | −0.821889253 | 10.08911092 | 62.02700461 | 3.38779E−15 | 4.27868E−13 |
| Rpl7 | 0.334243439 | 11.21182536 | 61.69813298 | 4.0036E−15 | 5.01722E−13 |
| Rpl27a | 0.292308559 | 11.73982066 | 61.44017246 | 4.56403E−15 | 5.67554E−13 |
| Rpl22 | 0.408207942 | 10.60705713 | 61.37209441 | 4.7246E−15 | 5.83037E−13 |
| Ptprc | −0.713573469 | 10.11543823 | 61.24768507 | 5.03277E−15 | 6.16362E−13 |
| Rps13 | 0.284620979 | 11.92518428 | 60.88657604 | 6.04597E−15 | 7.34881E−13 |
| Rpl5 | 0.439923342 | 10.56750057 | 60.78676801 | 6.3604E−15 | 7.6733E−13 |
| Slc25a3 | 0.594192956 | 9.949500334 | 60.65326175 | 6.80672E−15 | 8.15092E−13 |
| Rpl8 | 0.310489568 | 11.65215979 | 60.46534585 | 7.48853E−15 | 8.90144E−13 |
| Srgn | 0.449658173 | 11.37080589 | 60.20798913 | 8.53451E−15 | 1.00707E−12 |
| Rpl27-ps3 | 0.779626572 | 9.836237785 | 59.40764719 | 1.2817E−14 | 1.50145E−12 |
| Rpl39 | 0.322065417 | 11.77724389 | 59.04445308 | 1.5415E−14 | 1.7928E−12 |
| Rpl41 | 0.244533547 | 12.79519234 | 59.0064913 | 1.57153E−14 | 1.81466E−12 |
| Gm9493 | 0.537190778 | 10.33345076 | 58.58099131 | 1.95092E−14 | 2.23678E−12 |
| Rps4x | 0.289254484 | 11.84791468 | 58.37963616 | 2.16116E−14 | 2.46038E−12 |
| Gzmk | −1.778766406 | 9.363688173 | 58.22977163 | 2.33223E−14 | 2.63656E−12 |
| Chrne | 1.080079344 | 9.215523871 | 58.15710461 | 2.41999E−14 | 2.71677E−12 |
| Zfp36l2 | −1.196447923 | 9.814176688 | 57.88375668 | 2.78074E−14 | 3.10024E−12 |
| Rps5 | 0.281501081 | 12.08884857 | 57.86056343 | 2.81372E−14 | 3.11552E−12 |
| Rps8 | 0.329920366 | 11.59253946 | 57.82162114 | 2.86998E−14 | 3.15619E−12 |
| Ptprcap | −0.843038887 | 10.0401219 | 57.71648011 | 3.02756E−14 | 3.30699E−12 |
| Hmha1 | −0.896320622 | 9.750277921 | 57.60875529 | 3.19799E−14 | 3.46971E−12 |
| Smap2 | −1.082941862 | 9.515119635 | 57.58389709 | 3.23866E−14 | 3.49041E−12 |
| Serp1 | 0.703010004 | 9.615297449 | 57.37262964 | 3.6059E−14 | 3.86047E−12 |
| Lck | −0.584926841 | 10.3166673 | 57.19919538 | 3.93832E−14 | 4.18861E−12 |
| Arpc1b | −0.514495687 | 10.55363485 | 56.87151403 | 4.65236E−14 | 4.91569E−12 |
| Psmb8 | −0.547617393 | 10.43462296 | 56.15435567 | 6.6999E−14 | 7.03316E−12 |
| Tbc1d10c | −1.109015086 | 9.522686372 | 56.04149687 | 7.09574E−14 | 7.40063E−12 |
| Gm2000 | 0.754104213 | 9.643824227 | 55.94423 | 7.45562E−14 | 7.72613E−12 |
| Rps25 | 0.341236461 | 11.11865723 | 55.73810371 | 8.27978E−14 | 8.52554E−12 |
| Prr13 | −0.635092799 | 10.23790791 | 55.35597511 | 1.00564E−13 | 1.02893E−11 |
| Arf4 | 0.613490192 | 9.974773579 | 55.29863988 | 1.0354E−13 | 1.05272E−11 |
| Sp100 | −1.052989046 | 9.553710362 | 55.27939258 | 1.04559E−13 | 1.05644E−11 |
| Rpl11 | 0.271060468 | 11.70901048 | 55.22698927 | 1.07384E−13 | 1.07824E−11 |
| Alyref | −1.276590695 | 9.471546257 | 54.26812572 | 1.74916E−13 | 1.74549E−11 |
| Tnfsf11 | 1.195675235 | 9.546999328 | 54.14786369 | 1.85956E−13 | 1.84427E−11 |
| Hnrnpf | −0.542960485 | 10.40307656 | 54.04990147 | 1.95462E−13 | 1.92673E−11 |
| Rasgrp1 | 0.867495082 | 9.487696794 | 53.8710296 | 2.14091E−13 | 2.09758E−11 |
| Rps14 | 0.245646052 | 12.65498328 | 53.84935221 | 2.16466E−13 | 2.10807E−11 |
| Cd160 | −1.612019875 | 9.334774618 | 53.43431062 | 2.67386E−13 | 2.58836E−11 |
| Klrd1 | −1.530760278 | 9.372671712 | 53.33075396 | 2.81859E−13 | 2.71222E−11 |
| Slfn2 | −0.780872487 | 10.08513601 | 53.26722348 | 2.91124E−13 | 2.7848E−11 |
| Nt5e | 0.983503956 | 9.297373639 | 52.50026078 | 4.30191E−13 | 4.09087E−11 |
| Acbd7 | 0.727171497 | 9.16408021 | 52.47608183 | 4.3552E−13 | 4.11732E−11 |
| Rpl36 | 0.291113805 | 11.87411161 | 52.39297427 | 4.54346E−13 | 4.27033E−11 |
| Zrsr1 | 0.884462945 | 9.173204714 | 51.98113864 | 5.60363E−13 | 5.23632E−11 |
| Ero1l | 0.898463636 | 9.301152497 | 51.86195302 | 5.95431E−13 | 5.53203E−11 |
| Rab4a | 0.981396634 | 9.180878739 | 51.70674863 | 6.44409E−13 | 5.95286E−11 |
| Rpl14 | 0.276260435 | 11.87702722 | 51.33644975 | 7.78173E−13 | 7.125E−11 |
| Tnfrsf9 | 1.216186143 | 9.401994134 | 51.33156871 | 7.8011E−13 | 7.125E−11 |
| Ccl1 | 2.8701985 | 9.258088506 | 51.06468191 | 8.93719E−13 | 8.11678E−11 |
| Rpl10 | 0.340760182 | 11.48651267 | 51.00196212 | 9.22737E−13 | 8.3335E−11 |
| Rps26-ps1 | 0.834947975 | 9.373764532 | 50.54920683 | 1.16214E−12 | 1.04373E−10 |
| Rps27 | 0.313487317 | 13.23490088 | 50.2616202 | 1.34556E−12 | 1.20178E−10 |
| Rps17 | 0.442764705 | 10.61490628 | 50.23389231 | 1.3647E−12 | 1.21219E−10 |
| Gm11808 | 0.563530104 | 10.17399793 | 49.66892667 | 1.82006E−12 | 1.60782E−10 |
| Eprs | 0.923361989 | 9.384340903 | 49.48741908 | 1.99648E−12 | 1.75408E−10 |
| Ccr4 | 1.037365927 | 9.227399089 | 49.14947696 | 2.37182E−12 | 2.07258E−10 |
| Ptpn22 | −0.744716395 | 10.09359672 | 48.95338111 | 2.62119E−12 | 2.27818E−10 |
| Actr3 | −0.58253223 | 10.23419091 | 48.85186728 | 2.76042E−12 | 2.38637E−10 |
| Xist | 1.36070209 | 9.88830013 | 48.50687321 | 3.29133E−12 | 2.8302E−10 |
| Rpl28 | 0.392724036 | 10.72545285 | 48.30031575 | 3.65693E−12 | 3.12794E−10 |
| Lgals3 | −1.232792314 | 10.20014577 | 47.74671945 | 4.84995E−12 | 4.12654E−10 |
| Bcl2a1a | 1.056620671 | 9.252638693 | 47.53163715 | 5.41234E−12 | 4.58093E−10 |
| Eif2s3y | −1.186564598 | 9.297554666 | 47.39179745 | 5.81254E−12 | 4.89404E−10 |
| Anxa2 | −0.978635221 | 9.842357829 | 47.37611672 | 5.85922E−12 | 4.90778E−10 |
| Rpl18a | 0.234079517 | 12.84274109 | 47.34965143 | 5.93887E−12 | 4.94885E−10 |
| Fam49b | −0.709772908 | 9.757609288 | 47.31498589 | 6.04483E−12 | 5.01132E−10 |
| Cd40lg | 0.891889007 | 9.469095126 | 47.26266572 | 6.20835E−12 | 5.12062E−10 |
| Grk6 | −1.023874648 | 9.451805892 | 47.22787925 | 6.31952E−12 | 5.18585E−10 |
| Set | −0.828366713 | 9.773721763 | 47.01187204 | 7.0558E−12 | 5.76081E−10 |
| S100a4 | −1.423147096 | 9.866428203 | 46.98623023 | 7.14872E−12 | 5.80735E−10 |
| Arid5a | −0.881618443 | 9.656928977 | 46.45120687 | 9.39285E−12 | 7.59224E−10 |
| Itgb2 | −0.907516358 | 9.57508737 | 46.39812896 | 9.65076E−12 | 7.7619E−10 |
| Rpl27 | 0.289912917 | 11.47261069 | 46.31227214 | 1.0083E−11 | 8.06943E−10 |
| Pycard | −1.017137077 | 9.577060094 | 46.08847696 | 1.13031E−11 | 9.0013E−10 |
| Spty2d1 | 0.853051885 | 9.326966648 | 46.00769813 | 1.17789E−11 | 9.33422E−10 |
| Rhoc | 0.987511986 | 9.24424751 | 45.56307656 | 1.47802E−11 | 1.16554E−09 |
| Chst12 | −1.147202534 | 9.307931568 | 45.51891285 | 1.51172E−11 | 1.18633E−09 |
| Rps16 | 0.244105981 | 12.35138152 | 45.46537675 | 1.55361E−11 | 1.21332E−09 |
| Tmem64 | 1.093268199 | 9.248121955 | 45.0264485 | 1.94391E−11 | 1.51083E−09 |
| Irg1 | 1.166366825 | 9.171594961 | 45.00188351 | 1.96845E−11 | 1.52258E−09 |
| Rps11 | 0.248211858 | 12.26303884 | 44.94510537 | 2.02636E−11 | 1.55991E−09 |
| Klf6 | −1.036319624 | 9.897270148 | 44.77739178 | 2.20757E−11 | 1.68841E−09 |
| Gm6133 | 0.585153871 | 9.885540209 | 44.77154418 | 2.21417E−11 | 1.68841E−09 |
| Sik2 | 0.906902057 | 9.224898712 | 44.5694824 | 2.45488E−11 | 1.86318E−09 |
| Txk | −1.156179358 | 9.335431681 | 44.50879221 | 2.53217E−11 | 1.91286E−09 |
| Sarnp | −0.827594901 | 9.551872587 | 44.48372488 | 2.56481E−11 | 1.92849E−09 |
| Itm2c | 0.701342323 | 9.518100312 | 44.45278354 | 2.60566E−11 | 1.95015E−09 |
| Rpsa | 0.289184479 | 11.60004787 | 43.86706712 | 3.51453E−11 | 2.61824E−09 |
| Rps3 | 0.225942304 | 11.96703538 | 43.69704055 | 3.83351E−11 | 2.84278E−09 |
| Ssu72 | −0.929150446 | 9.444716001 | 43.50697036 | 4.22452E−11 | 3.10425E−09 |
| Myo1f | −1.049004748 | 9.304637414 | 43.51237732 | 4.21286E−11 | 3.10425E−09 |
| Gnb2l1 | 0.366860437 | 10.54001608 | 43.15062564 | 5.06837E−11 | 3.70748E−09 |
| Pitpna | −0.910485352 | 9.510883804 | 43.13747045 | 5.10256E−11 | 3.71568E−09 |
| Satb1 | 0.745579653 | 9.757025507 | 42.83945985 | 5.94214E−11 | 4.30765E−09 |
| Gna13 | 0.591042418 | 9.985105007 | 42.61542934 | 6.66317E−11 | 4.80879E−09 |
| Fth1 | 0.373781364 | 11.58470725 | 42.59490598 | 6.73345E−11 | 4.83791E−09 |
| Gabpb1 | 0.835408177 | 9.477998587 | 42.55353038 | 6.8774E−11 | 4.91947E−09 |
| Rpl15 | 0.344396098 | 10.73579943 | 42.1315169 | 8.53367E−11 | 6.07733E−09 |
| Clic1 | −0.535683728 | 10.32552855 | 42.07634585 | 8.77786E−11 | 6.22381E−09 |
| Ifitm2 | 0.977337784 | 10.68417408 | 42.03733753 | 8.95472E−11 | 6.32148E−09 |
| Tgfb1 | 0.671608382 | 9.751051801 | 41.86242946 | 9.79263E−11 | 6.88294E−09 |
| Dnajb6 | 0.49894243 | 10.02351389 | 41.74117381 | 1.04192E−10 | 7.2916E−09 |
| Rpl32 | 0.23664982 | 12.44732842 | 41.59727176 | 1.1215E−10 | 7.8147E−09 |
| Ifng | −1.717507241 | 9.686677357 | 41.44701983 | 1.21109E−10 | 8.40278E−09 |
| Rpl7a-ps5 | 0.835650714 | 9.342537523 | 41.40691188 | 1.2362E−10 | 8.54031E−09 |
| Ctsw | −0.729741102 | 9.943517783 | 41.35806506 | 1.26747E−10 | 8.6871E−09 |
| Plcxd2 | −1.101786892 | 9.360411229 | 41.3569629 | 1.26819E−10 | 8.6871E−09 |
| Ssr4 | 0.570246619 | 9.653687486 | 41.18546566 | 1.38448E−10 | 9.44369E−09 |
| Ptp4a1 | 0.58771196 | 9.696533105 | 41.0105522 | 1.5141E−10 | 1.02844E−08 |
| Cnn2 | −0.693314256 | 9.902894206 | 40.62768913 | 1.84178E−10 | 1.24578E−08 |
| Bcl2a1d | 0.63858231 | 9.947932761 | 40.61758242 | 1.85133E−10 | 1.24703E−08 |
| Rps18 | 0.241335122 | 12.47331521 | 40.48050608 | 1.98588E−10 | 1.3321E−08 |
| Mndal | −0.841737872 | 9.573225112 | 40.46492083 | 2.00178E−10 | 1.33722E−08 |
| Eef1b2 | 0.442727395 | 10.07535887 | 40.41960733 | 2.04875E−10 | 1.36296E−08 |
| Cbx3 | −1.070891558 | 9.518054426 | 40.379645 | 2.09108E−10 | 1.38543E−08 |
| Capzb | −0.523447722 | 10.15887429 | 40.2640148 | 2.21858E−10 | 1.4639E−08 |
| Nck2 | 0.781410509 | 9.181413604 | 40.15865483 | 2.34152E−10 | 1.53874E−08 |
| Ywhah | −0.731211687 | 9.721925417 | 40.03745907 | 2.49139E−10 | 1.6306E−08 |
| H2afy | −0.620358 | 9.930108471 | 39.94698726 | 2.6095E−10 | 1.70101E−08 |
| Rab26os | 0.808791054 | 9.199100322 | 39.7121166 | 2.94294E−10 | 1.91067E−08 |
| Esm1 | −1.379695329 | 9.307914728 | 39.65850624 | 3.02484E−10 | 1.95598E−08 |
| Rbm3 | −0.469825784 | 10.73094825 | 39.54036966 | 3.21347E−10 | 2.06968E−08 |
| Stk4 | 0.758728411 | 9.393514541 | 39.37433961 | 3.49865E−10 | 2.24441E−08 |
| Usmg5 | 0.4807315 | 10.06857914 | 39.35044395 | 3.54172E−10 | 2.26306E−08 |
| Ppp1cc | 0.49871973 | 10.14022773 | 39.20971704 | 3.80641E−10 | 2.42262E−08 |
| Efhd2 | −0.874966943 | 9.465518512 | 39.00726633 | 4.22231E−10 | 2.67678E−08 |
| 1810058I24Rik | −0.854214534 | 9.463226674 | 38.99196176 | 4.25554E−10 | 2.68731E−08 |
| Tarm1 | 0.349845066 | 9.158101881 | 38.98305009 | 4.27501E−10 | 2.6891E−08 |
| Ifi203 | −0.937675411 | 9.466821262 | 38.51022019 | 5.44677E−10 | 3.41289E−08 |
| Nfkbie | 0.776225335 | 9.398337686 | 38.4527893 | 5.60944E−10 | 3.50124E−08 |
| Sept7 | −0.781596305 | 9.59369479 | 38.35737928 | 5.89049E−10 | 3.66253E−08 |
| Rpl37 | 0.232492992 | 12.07300053 | 38.08056479 | 6.78829E−10 | 4.20458E−08 |
| Riok1 | 0.83556849 | 9.379908874 | 38.03508968 | 6.94837E−10 | 4.2873E−08 |
| Bin2 | −0.817818919 | 9.612865616 | 37.72529296 | 8.14417E−10 | 5.00603E−08 |
| Scamp3 | −0.845996416 | 9.450290953 | 37.57880894 | 8.77931E−10 | 5.376E−08 |
| Cd69 | −1.012008029 | 9.886570775 | 37.5387415 | 8.96152E−10 | 5.46687E−08 |

TABLE 3M
LPL-0 Ctrl vs. KO

| GeneSymbol | logFC | logCPM | LR | PValue | FDR |
|---|---|---|---|---|---|
| Hspala | −3.825403493 | 11.185063 | 645.7540972 | 1.8729E−142 | 1.5139E−138 |
| Dnajb1 | −2.99161603 | 10.75617704 | 646.5297575 | 1.2701E−142 | 1.5139E−138 |
| Ubb | −0.822066561 | 12.91815268 | 545.542542 | 1.174E−120 | 6.3264E−117 |
| Hsp90aa1 | −1.66428051 | 10.89396783 | 533.0215097 | 6.2174E−118 | 2.5127E−114 |
| Hsp90ab1 | −1.143395141 | 11.28555087 | 489.4360578 | 1.8903E−108 | 6.1116E−105 |
| Malat1 | −0.834514356 | 15.05517862 | 480.5825476 | 1.5958E−106 | 4.2997E−103 |
| Dnaja1 | −1.340678809 | 11.00180648 | 424.3156706 | 2.80608E−94 | 6.48045E−91 |
| Hspa8 | −0.841935877 | 12.34011222 | 389.8667875 | 8.84867E−87 | 1.78809E−83 |
| Hsph1 | −1.682363639 | 9.992887058 | 286.2067369 | 3.33497E−64 | 5.99036E−61 |
| Hspe1 | −1.262621525 | 10.53897111 | 284.1409882 | 9.40213E−64 | 1.51995E−60 |
| Jund | −0.757938516 | 12.16529797 | 274.712105 | 1.06649E−61 | 1.56736E−58 |
| Hspa1b | −1.813713236 | 9.880656558 | 192.3062295 | 9.97577E−44 | 1.3439E−40 |
| Rpl35 | 0.479975201 | 11.30628837 | 180.4188355 | 3.9262E−41 | 4.88238E−38 |
| Hspd1 | −1.057296091 | 10.15136767 | 143.4046976 | 4.79454E−33 | 5.53632E−30 |
| Fosb | −1.249403116 | 9.939486915 | 142.5770614 | 7.27286E−33 | 7.83821E−30 |
| Rbm3 | 0.507343098 | 10.86474429 | 133.5193484 | 6.96011E−31 | 7.03232E−28 |
| Rpl27-ps3 | 0.733939207 | 10.1630502 | 126.7813677 | 2.07409E−29 | 1.97234E−26 |
| Eef2 | 0.365125682 | 11.39715697 | 125.7387136 | 3.50754E−29 | 3.15016E−26 |
| Zfp36l2 | 0.861788833 | 10.24225162 | 118.3228897 | 1.4734E−27 | 1.25363E−24 |
| Rpl9-ps6 | 0.665300057 | 10.19128936 | 115.4607797 | 6.23806E−27 | 5.04223E−24 |
| Gm8730 | 0.501024919 | 10.67600704 | 115.0937451 | 7.50637E−27 | 5.77848E−24 |
| Rpl22l1 | 0.470188136 | 11.02604766 | 114.7398327 | 8.97303E−27 | 6.59354E−24 |
| Rpl32 | 0.276004844 | 12.43016279 | 112.5208191 | 2.74765E−26 | 1.93124E−23 |
| Rpl29 | 0.373802656 | 11.30509047 | 110.5431633 | 7.45054E−26 | 5.01856E−23 |
| Rpl6l | 0.518002583 | 10.48321852 | 101.9448287 | 5.70898E−24 | 3.69166E−21 |
| Junb | −0.466016075 | 12.18505258 | 98.00914384 | 4.16455E−23 | 2.58939E−20 |
| Rplp0 | 0.249731984 | 12.38724467 | 96.03494503 | 1.12873E−22 | 6.75819E−20 |
| Chchd2 | −0.452053053 | 10.66873479 | 94.44852779 | 2.51549E−22 | 1.45234E−19 |
| Rgcc | −1.083204205 | 10.02460697 | 91.73836745 | 9.89281E−22 | 5.51473E−19 |
| Rpl18 | 0.254662339 | 11.84337657 | 86.96767873 | 1.10309E−20 | 5.9442E−18 |
| Ddx5 | −0.364346068 | 11.32206669 | 82.20282545 | 1.22814E−19 | 6.40456E−17 |
| Cacybp | −0.759965312 | 9.974878831 | 81.02462915 | 2.22922E−19 | 1.12617E−16 |
| Lmna | −0.896054608 | 10.10046144 | 77.25333728 | 1.50377E−18 | 7.36666E−16 |
| Gm9843 | 0.424425093 | 10.7167237 | 76.61162997 | 2.0811E−18 | 9.89504E−16 |
| Cdkn1a | −0.802900828 | 10.20349727 | 74.35181316 | 6.53652E−18 | 3.01913E−15 |
| Rps18-ps3 | 0.682699869 | 9.936337431 | 73.30477018 | 1.11099E−17 | 4.98897E−15 |
| Cd160 | 0.933946071 | 9.842212445 | 72.70824087 | 1.50305E−17 | 6.56712E−15 |
| Gzma | 1.209224468 | 10.74237183 | 70.50937153 | 4.58085E−17 | 1.94879E−14 |
| Rpl13a | 0.174478619 | 13.13700027 | 69.64459685 | 7.1013E−17 | 2.94358E−14 |
| Odc1 | −0.554763585 | 10.71341295 | 69.11801272 | 9.27444E−17 | 3.74827E−14 |
| Atf3 | −0.87943707 | 9.821862051 | 68.15164877 | 1.51393E−16 | 5.96931E−14 |
| Hspa5 | −0.432882858 | 11.06371391 | 66.89489047 | 2.86376E−16 | 1.10228E−13 |
| Gm9493 | 0.415290628 | 10.58239681 | 66.56840727 | 3.37959E−16 | 1.27057E−13 |
| Epsti1 | 0.518154471 | 10.33595192 | 65.73125853 | 5.16797E−16 | 1.89876E−13 |
| Eif3f | 0.334919369 | 10.83491205 | 65.21112438 | 6.72886E−16 | 2.41731E−13 |
| Kdm6b | −0.502890922 | 10.78968221 | 64.63386514 | 9.01918E−16 | 3.16965E−13 |
| S100a4 | 0.92404098 | 10.00434996 | 63.64817399 | 1.48747E−15 | 5.11626E−13 |
| Ccl5 | 0.726508356 | 13.19304376 | 63.37342879 | 1.71009E−15 | 5.75945E−13 |
| Fam110a | −0.691881356 | 9.98944843 | 62.94516444 | 2.12542E−15 | 7.01216E−13 |
| Ubald2 | −0.420100725 | 10.91847462 | 61.80199233 | 3.7979E−15 | 1.22794E−12 |
| Rps11 | 0.206504838 | 12.2866429 | 60.53858145 | 7.21504E−15 | 2.28703E−12 |
| Rpl3 | 0.207637882 | 12.17538078 | 59.964028 | 9.6607E−15 | 3.00336E−12 |
| Limd2 | 0.46356712 | 10.41509448 | 58.64505882 | 1.88842E−14 | 5.76003E−12 |
| Rpl36-ps3 | 0.600107604 | 9.956192534 | 58.34228896 | 2.20258E−14 | 6.47399E−12 |
| Rpl10-ps3 | 0.588938103 | 10.00304739 | 58.37673424 | 2.16435E−14 | 6.47399E−12 |
| Gm2000 | 0.552978434 | 10.04573455 | 58.2135153 | 2.35158E−14 | 6.78851E−12 |
| Crem | −0.555480946 | 10.37476775 | 58.14841144 | 2.4307E−14 | 6.89382E−12 |
| Satb1 | 0.611847835 | 10.18239119 | 57.22454132 | 3.88789E−14 | 1.08365E−11 |
| H2-Q6 | −0.52349562 | 10.11218978 | 55.63954367 | 8.70547E−14 | 2.3853E−11 |
| Rac2 | 0.278484799 | 11.27241752 | 55.32603535 | 1.02107E−13 | 2.75111E−11 |
| Rpl24 | 0.215735106 | 11.83074602 | 55.08988222 | 1.15142E−13 | 3.05145E−11 |
| H2-K1 | −0.242269234 | 11.67413497 | 54.76242772 | 1.36016E−13 | 3.54652E−11 |
| Ifrd1 | −0.490058089 | 10.42505666 | 54.57837129 | 1.49371E−13 | 3.8329E−11 |
| Eno1 | 0.480893285 | 10.2518593 | 54.42764048 | 1.61278E−13 | 4.07379E−11 |
| Rgs10 | 0.584571469 | 10.06092484 | 53.57232916 | 2.49245E−13 | 6.19892E−11 |
| Ptprcap | 0.462562077 | 10.30191657 | 52.78189829 | 3.72721E−13 | 9.1294E−11 |
| Rpl15 | 0.285131556 | 10.91329125 | 52.45039836 | 4.41253E−13 | 1.06467E−10 |
| Fus | −0.436843347 | 10.31636377 | 51.74106738 | 6.33243E−13 | 1.50544E−10 |
| Apobec3 | 0.580477188 | 9.941216731 | 51.13331646 | 8.6301E−13 | 2.02195E−10 |
| Wdr89 | 0.360242636 | 10.60823124 | 51.02458523 | 9.12163E−13 | 2.10658E−10 |
| Prr7 | −0.512955962 | 10.2326335 | 49.73875281 | 1.75642E−12 | 3.9992E−10 |
| Cd52 | 0.261151609 | 11.82769179 | 49.55775305 | 1.92617E−12 | 4.32479E−10 |
| Actg1 | 0.279020484 | 11.92138436 | 49.3310168 | 2.16217E−12 | 4.78816E−10 |
| Fth1 | −0.254879504 | 11.68995556 | 49.06749956 | 2.47304E−12 | 5.40259E−10 |
| Rpsa | 0.221212609 | 11.63951649 | 48.74914351 | 2.90886E−12 | 6.26994E−10 |
| Hmgb2 | 0.380619263 | 10.77568432 | 47.80559174 | 4.70648E−12 | 1.00112E−09 |
| Gm10073 | 0.50591008 | 10.0938124 | 47.04184308 | 6.94873E−12 | 1.44648E−09 |
| Ltb | 0.46912106 | 10.47411608 | 47.03326566 | 6.9792E−12 | 1.44648E−09 |
| Rpl7a-ps5 | 0.593867674 | 9.839399427 | 46.10042491 | 1.12344E−11 | 2.29893E−09 |
| Bag3 | −0.676847869 | 9.761603714 | 45.80267572 | 1.30785E−11 | 2.64283E−09 |
| Rgs2 | −0.602713321 | 10.33738405 | 45.56039753 | 1.48004E−11 | 2.95387E−09 |
| mt-Nd1 | −0.27246384 | 11.38477942 | 44.99146781 | 1.97895E−11 | 3.90142E−09 |
| Selplg | 0.361247701 | 10.47107152 | 44.61343362 | 2.40039E−11 | 4.67527E−09 |
| Rps12-ps3 | 0.429501896 | 10.24219127 | 44.54830901 | 2.48158E−11 | 4.77585E−09 |
| Itpkb | −0.536976006 | 10.03354471 | 43.56508254 | 4.10091E−11 | 7.79945E−09 |
| Lcp1 | 0.358521569 | 10.53990253 | 43.01457134 | 5.43337E−11 | 1.02135E−08 |
| Rps10 | 0.229905401 | 11.28625925 | 42.82120188 | 5.99786E−11 | 1.1145E−08 |
| Shisa5 | 0.253218689 | 11.20411978 | 42.66916005 | 6.48263E−11 | 1.19089E−08 |
| Cd7 | 0.905512708 | 9.951566624 | 42.42624652 | 7.33984E−11 | 1.33321E−08 |
| Btf3 | 0.265152023 | 10.86771989 | 42.36584762 | 7.57004E−11 | 1.35975E−08 |
| Eif1 | −0.185728716 | 11.94746374 | 41.72703666 | 1.04948E−10 | 1.86438E−08 |
| Gm10076 | 0.471465459 | 10.05875362 | 41.19742905 | 1.37603E−10 | 2.41793E−08 |
| Arhgdib | 0.291487089 | 10.83943531 | 41.11935386 | 1.43211E−10 | 2.48941E−08 |
| Hmha1 | 0.438419112 | 10.13557056 | 40.84407814 | 1.64872E−10 | 2.83545E−08 |
| Cnn2 | 0.404936534 | 10.25178545 | 40.75026013 | 1.7298E−10 | 2.94358E−08 |
| Rps6 | 0.159056414 | 12.23920984 | 40.68855047 | 1.7853E−10 | 3.00637E−08 |
| Itgae | 0.677929929 | 9.8104067 | 40.59773584 | 1.87023E−10 | 3.11693E−08 |
| Fam169b | 0.616416705 | 9.830231324 | 40.37061673 | 2.10077E−10 | 3.46541E−08 |
| Rps3 | 0.163758935 | 11.98064091 | 40.05254335 | 2.47223E−10 | 4.03697E−08 |
| Slc25a5 | 0.318042836 | 10.56075782 | 39.92967324 | 2.63273E−10 | 4.25607E−08 |
| B2m | −0.138337493 | 12.76834333 | 39.80722236 | 2.80307E−10 | 4.48657E−08 |
| Cox4i1 | 0.260138703 | 10.88297738 | 39.75543771 | 2.87838E−10 | 4.56195E−08 |
| Nr3c1 | −0.562192365 | 9.957485177 | 39.24465631 | 3.7389E−10 | 5.86826E−08 |
| Dedd2 | −0.579408471 | 9.822902175 | 38.8738958 | 4.52086E−10 | 7.02733E−08 |
| Ctla4 | −0.411701514 | 10.49036865 | 38.58391272 | 5.24495E−10 | 8.07522E−08 |
| Eif4a1 | −0.320154261 | 10.60075837 | 38.51060462 | 5.4457E−10 | 8.3052E−08 |
| Gltscr2 | 0.29072519 | 10.57741115 | 38.07787817 | 6.79765E−10 | 1.02702E−07 |
| Il4ra | −0.505217024 | 10.00229239 | 37.82530316 | 7.73717E−10 | 1.15814E−07 |
| Ctsw | 0.38528809 | 10.29455423 | 37.78999709 | 7.87847E−10 | 1.16847E−07 |

TABLE 3N
LPL-1 Ctrl vs. KO

| GeneSymbol | logFC | logCPM | LR | PValue | FDR |
|---|---|---|---|---|---|
| Gm9843 | 0.873546513 | 10.7197657 | 595.3267532 | 1.7389E−131 | 2.8111E−127 |
| Rps27rt | 0.66236952 | 11.52848942 | 503.5200918 | 1.6296E−111 | 1.3172E−107 |
| Rpl27-ps3 | 1.093919351 | 10.0373069 | 498.0920313 | 2.4723E−110 | 1.3323E−106 |
| Rpl23a-ps3 | 0.706965217 | 10.91927368 | 429.8668325 | 1.73729E−95 | 7.02125E−92 |
| Gm8730 | 0.825757188 | 10.42923933 | 412.6161475 | 9.87786E−92 | 3.19371E−88 |
| Wdr89 | 0.707321788 | 10.74901788 | 406.5956198 | 2.0192E−90 | 5.44041E−87 |
| Rpl6l | 0.823496109 | 10.31624825 | 405.0286107 | 4.4288E−90 | 1.0228E−86 |
| Rpl9-ps6 | 0.947575445 | 10.03468303 | 373.6299221 | 3.0329E−83 | 6.12873E−80 |
| Gm11808 | 0.822511826 | 10.38635863 | 367.3621485 | 7.02331E−82 | 1.26154E−78 |
| Hsp90aa1 | −1.123206902 | 11.09080685 | 278.1373372 | 1.91209E−62 | 3.09108E−59 |
| Hsp90ab1 | −0.798418537 | 11.45738705 | 277.329778 | 2.86743E−62 | 4.21409E−59 |
| Gm9493 | 0.669365375 | 10.45191966 | 267.6617764 | 3.66879E−60 | 4.94247E−57 |
| Ubb | −0.506909232 | 13.07324632 | 266.81113 | 5.62244E−60 | 6.99172E−57 |
| Hspa8 | −0.727896974 | 12.56632486 | 263.6950407 | 2.68601E−59 | 3.10157E−56 |
| Gm2000 | 0.978296872 | 9.727076162 | 255.8188211 | 1.3994E−57 | 1.50818E−54 |
| Gm6133 | 0.812351209 | 10.01774141 | 251.4273837 | 1.26841E−56 | 1.28157E−53 |
| Gm10073 | 0.884912534 | 9.850676041 | 242.7892061 | 9.69501E−55 | 9.21939E−52 |
| Uba52 | 0.636432939 | 10.43662801 | 223.7341252 | 1.38646E−50 | 1.24519E−47 |
| Rpl35 | 0.477127259 | 11.02808234 | 202.7745708 | 5.18058E−46 | 4.40786E−43 |
| Dnajb1 | −1.547344284 | 10.51482327 | 202.0042358 | 7.6291E−46 | 6.1666E−43 |
| Hspa1a | −2.383248273 | 10.75818044 | 185.9121194 | 2.48129E−42 | 1.91012E−39 |
| Gm10076 | 0.89711374 | 9.682747299 | 184.0884505 | 6.20583E−42 | 4.56016E−39 |
| Gm42418 | 1.003104559 | 9.481098691 | 164.784568 | 1.01946E−37 | 7.16548E−35 |
| Rpl36-ps3 | 0.865876631 | 9.656996395 | 163.4954362 | 1.94979E−37 | 1.31335E−34 |
| Lgals1 | −0.600108402 | 11.14033101 | 159.5092037 | 1.44838E−36 | 9.3658E−34 |
| Tmsb4x | −0.269818422 | 14.02813382 | 157.946803 | 3.17881E−36 | 1.97649E−33 |
| Rps18-ps3 | 0.841244657 | 9.655928496 | 157.4523302 | 4.07672E−36 | 2.4409E−33 |
| Rpl13-ps3 | 0.736506126 | 9.797721135 | 150.5036043 | 1.3455E−34 | 7.76836E−32 |
| Dnaja1 | −0.748586019 | 11.1271812 | 146.0105043 | 1.29135E−33 | 7.19862E−31 |
| Chchd2 | −0.473494251 | 10.6318899 | 141.6095467 | 1.18374E−32 | 6.37879E−30 |
| Hspe1 | −0.829543845 | 10.55971604 | 128.4300367 | 9.03775E−30 | 4.71304E−27 |
| Rpl10-ps3 | 0.71087493 | 9.678396441 | 125.6681744 | 3.63446E−29 | 1.83609E−26 |
| Gm10036 | 0.758004858 | 9.644830032 | 125.5491133 | 3.8592E−29 | 1.89054E−26 |
| Fosb | −1.108477341 | 9.588519321 | 113.8078997 | 1.43564E−26 | 6.82607E−24 |
| Rps12-ps3 | 0.548511272 | 10.07738295 | 110.8914873 | 6.24998E−26 | 2.88678E−23 |
| Malat1 | −0.323252323 | 14.85278289 | 110.8000382 | 6.54504E−26 | 2.93909E−23 |
| Lmna | −0.973775479 | 10.04980932 | 110.2750726 | 8.52945E−26 | 3.72668E−23 |
| Zfp36l2 | 0.805436207 | 9.84848683 | 109.5140802 | 1.25212E−25 | 5.32678E−23 |
| Bcl2a1a | 0.944045562 | 9.35889223 | 108.9881407 | 1.6326E−25 | 6.76733E−23 |
| Gm26917 | 0.725412379 | 9.615524653 | 105.7709173 | 8.27715E−25 | 3.34521E−22 |
| Rps27 | 0.173150148 | 13.46009077 | 101.4527607 | 7.31882E−24 | 2.88576E−21 |
| Gm10263 | 0.861643794 | 9.385525775 | 98.05106258 | 4.07732E−23 | 1.56938E−20 |
| AY036118 | 0.845409107 | 9.416058201 | 95.63098079 | 1.38423E−22 | 5.20407E−20 |
| Rps28 | 0.25062322 | 11.89396748 | 94.87741825 | 2.02547E−22 | 7.44177E−20 |
| Cd160 | 1.023014317 | 9.339544002 | 94.02040194 | 3.12288E−22 | 1.12188E−19 |
| Hsph1 | −0.968446806 | 9.627387402 | 89.34964071 | 3.30852E−21 | 1.16273E−18 |
| Crip1 | −0.674587136 | 11.33426017 | 84.59617783 | 3.65972E−20 | 1.25879E−17 |
| Rpl7a-ps5 | 0.725809858 | 9.475578047 | 82.78303389 | 9.15726E−20 | 3.08409E−17 |
| Rgcc | −0.973619288 | 9.813843394 | 81.09113895 | 2.15544E−19 | 7.1112E−17 |
| Hspa1b | −1.064974481 | 9.37693802 | 77.03025429 | 1.68359E−18 | 5.44338E−16 |
| Gm10250 | 0.721261574 | 9.432548691 | 75.60196203 | 3.47017E−18 | 1.09997E−15 |
| B2m | −0.163151274 | 12.84650271 | 72.80837186 | 1.4287E−17 | 4.44159E−15 |
| Rps26-ps1 | 0.661606304 | 9.479500689 | 69.45313485 | 7.82519E−17 | 2.38683E−14 |
| Jund | −0.386316433 | 11.96429312 | 68.4310993 | 1.31389E−16 | 3.9334E−14 |
| Sh3bgrl3 | −0.284689974 | 11.2049149 | 67.6776059 | 1.92538E−16 | 5.65922E−14 |
| Rpl15 | 0.290483657 | 10.7193633 | 66.70193998 | 3.15824E−16 | 9.11717E−14 |
| H2-D1 | −0.168989587 | 12.4810284 | 65.49651581 | 5.82168E−16 | 1.65111E−13 |
| Rpl27 | 0.192059664 | 11.58272697 | 62.42809188 | 2.76353E−15 | 7.70263E−13 |
| Rpl38 | 0.175857657 | 11.85262055 | 59.68977327 | 1.11052E−14 | 3.04283E−12 |
| Ly6a | −0.476835045 | 10.45808259 | 58.56441313 | 1.96743E−14 | 5.30091E−12 |
| Gm8186 | 0.616545383 | 9.46968788 | 58.47837647 | 2.05538E−14 | 5.44708E−12 |
| Eef2 | 0.221539535 | 11.27929437 | 57.33667929 | 3.67242E−14 | 9.57554E−12 |
| Gzma | 0.973652374 | 10.76243447 | 56.49530585 | 5.63327E−14 | 1.44551E−11 |
| Hspa5 | −0.308894911 | 11.15772526 | 56.45869742 | 5.73913E−14 | 1.44967E−11 |
| Cd7 | 1.070388028 | 9.401826218 | 56.08605501 | 6.93674E−14 | 1.72522E−11 |
| Itgae | 0.757627092 | 9.327390666 | 54.75135772 | 1.36785E−13 | 3.3504E−11 |
| Eif3f | 0.271500969 | 10.59751999 | 54.44295273 | 1.60027E−13 | 3.86118E−11 |
| Gm9844 | 0.524290137 | 9.613183527 | 53.29612718 | 2.86872E−13 | 6.81995E−11 |
| Calm1 | −0.263318971 | 10.92853481 | 52.42032047 | 4.48064E−13 | 1.04977E−10 |
| Odc1 | −0.476042872 | 10.65279357 | 52.07968366 | 5.32935E−13 | 1.23077E−10 |
| Ly6e | −0.272932191 | 10.89033101 | 51.71546392 | 6.41555E−13 | 1.46076E−10 |
| Rps29 | 0.164848186 | 12.37594571 | 51.4604536 | 7.30539E−13 | 1.64026E−10 |
| Btg2 | 0.336367777 | 10.74241813 | 51.38924928 | 7.57522E−13 | 1.67755E−10 |
| Rgs2 | −0.487746639 | 10.60753305 | 50.89975769 | 9.72056E−13 | 2.12355E−10 |
| Ccl5 | 0.536522565 | 13.43614334 | 49.52632899 | 1.95727E−12 | 4.21884E−10 |
| Manf | −0.477853237 | 9.744077147 | 48.59281074 | 3.15022E−12 | 6.70085E−10 |
| Cacybp | −0.581354688 | 9.602687269 | 48.49367478 | 3.31356E−12 | 6.95675E−10 |
| Bcl2a1d | 0.434412334 | 10.08987012 | 48.36666838 | 3.53526E−12 | 7.32706E−10 |
| Gm42726 | 0.594380731 | 9.300818507 | 47.76332445 | 4.80905E−12 | 9.84089E−10 |
| Sertad1 | −0.609477867 | 9.448224564 | 45.15697193 | 1.81857E−11 | 3.67488E−09 |
| mt-Nd1 | −0.233531752 | 11.10794853 | 44.9507962 | 2.02048E−11 | 4.03248E−09 |
| Hspd1 | −0.577954866 | 9.977292502 | 44.91936291 | 2.05318E−11 | 4.04776E−09 |
| Ctla2a | −0.562767996 | 10.740003 | 44.73531038 | 2.25553E−11 | 4.39312E−09 |
| Rpl32 | 0.141395026 | 12.38187309 | 44.34948815 | 2.74684E−11 | 5.28636E−09 |
| Tubb4b | −0.503312119 | 9.606172658 | 42.96612383 | 5.5696E−11 | 1.05927E−08 |
| Stk17b | 0.337542743 | 10.09370635 | 42.23007718 | 8.11424E−11 | 1.52529E−08 |
| Myl12a | −0.257568889 | 10.62761221 | 41.55518391 | 1.1459E−10 | 2.12927E−08 |
| Apobec3 | 0.551355515 | 9.466440219 | 41.0441852 | 1.48826E−10 | 2.73401E−08 |
| H2-K1 | −0.161175353 | 11.86466227 | 40.58872572 | 1.87888E−10 | 3.4128E−08 |
| Actb | −0.289918864 | 11.94107968 | 39.18869365 | 3.84762E−10 | 6.91118E−08 |
| Cfl1 | −0.207073499 | 10.90597825 | 38.85100187 | 4.57419E−10 | 8.12597E−08 |
| Dynll1 | −0.347573679 | 10.0825626 | 37.98225887 | 7.13909E−10 | 1.25252E−07 |

TABLE 3O
LPL-2 Ctrl vs. KO

| GeneSymbol | logFC | logCPM | LR | PValue | FDR |
|---|---|---|---|---|---|
| Dnajb1 | 2.544628253 | 9.957159242 | 547.4793005 | 4.4499E−121 | 7.1937E−117 |
| Ubb | −0.719181201 | 12.62430285 | 513.9811277 | 8.6306E−114 | 6.9761E−110 |
| Hspa1a | −4.265472016 | 10.29821772 | 492.9716733 | 3.2152E−109 | 1.7326E−105 |
| Hsp90aa1 | −1.538726518 | 10.40571986 | 452.2223245 | 2.3685E−100 | 9.57241E−97 |
| Hsp90ab1 | −1.044511532 | 10.87430694 | 427.8486026 | 4.77686E−95 | 1.54445E−91 |
| Malat1 | −0.78769936 | 14.29544248 | 389.368725 | 1.13581E−86 | 3.06026E−83 |
| Dnaja1 | −1.160967215 | 10.50668275 | 365.4817217 | 1.80293E−81 | 4.16374E−78 |
| Lmna | −1.944600206 | 9.721553983 | 326.4307695 | 5.75683E−73 | 1.16331E−69 |
| Hspa8 | −0.679127098 | 12.09728281 | 319.2408275 | 2.11966E−71 | 3.80738E−68 |
| Gm8730 | 0.782167056 | 10.45597089 | 266.3217383 | 7.18771E−60 | 1.16196E−56 |
| Rpl27-ps3 | 1.052879725 | 9.707945918 | 234.7431502 | 5.50789E−53 | 8.0946E−50 |
| Rgcc | −1.710983657 | 9.519354512 | 214.546025 | 1.39991E−48 | 1.88592E−45 |
| Jund | −0.675872249 | 11.72419218 | 198.2604689 | 5.00547E−45 | 6.22449E−42 |
| Rpl35 | 0.509475196 | 11.16221273 | 190.7930532 | 2.13416E−43 | 2.46434E−40 |
| Gm10076 | 1.043288877 | 9.547149104 | 169.9338009 | 7.64936E−39 | 8.24397E−36 |
| Hspe1 | −0.935945148 | 9.917574254 | 149.4990174 | 2.23082E−34 | 2.25396E−31 |
| Rbm3 | 0.521950766 | 10.83382101 | 146.4106829 | 1.05575E−33 | 1.00395E−30 |
| Hspa1b | −1.576600791 | 9.179262147 | 137.5708581 | 9.04572E−32 | 8.12406E−29 |
| Rpl6l | 0.618995521 | 10.05911577 | 131.643752 | 1.79029E−30 | 1.52326E−27 |
| Rpl9-ps6 | 0.729047513 | 9.70611516 | 124.801031 | 5.62624E−29 | 4.54769E−26 |
| Plac8 | 1.004128708 | 10.71893409 | 122.0396335 | 2.26272E−28 | 1.74187E−25 |
| Rpl10-ps3 | 0.891923046 | 9.473250861 | 119.5256446 | 8.03499E−28 | 5.90425E−25 |
| Rplp0 | 0.287615554 | 12.48158976 | 116.701297 | 3.33728E−27 | 2.34567E−24 |
| Gm2000 | 0.840498181 | 9.539843664 | 112.8076916 | 2.37752E−26 | 1.60146E−23 |
| AY036118 | 1.145148849 | 9.18956426 | 110.8745505 | 6.30361E−26 | 3.91939E−23 |
| Rps11 | 0.296289107 | 12.28172419 | 110.9391621 | 6.10147E−26 | 3.91939E−23 |
| Gm9843 | 0.526758847 | 10.31729062 | 106.9052174 | 4.66978E−25 | 2.79599E−22 |
| Crip1 | −0.542787363 | 12.45883867 | 104.3227143 | 1.71907E−24 | 9.92517E−22 |
| Atf3 | −1.251293523 | 9.18931171 | 99.85032275 | 1.64361E−23 | 9.16225E−21 |
| Fosb | −1.291370796 | 9.247423493 | 98.57840948 | 3.12407E−23 | 1.68346E−20 |
| Ccl5 | 1.130393144 | 13.95145733 | 98.22916805 | 3.7266E−23 | 1.94336E−20 |
| H2-D1 | −0.246378131 | 12.31206309 | 88.35060601 | 5.48228E−21 | 2.76958E−18 |
| Gzma | 1.72506778 | 11.40703532 | 87.10004234 | 1.03168E−20 | 5.054E−18 |
| Capg | −0.678811855 | 9.911087478 | 86.82217942 | 1.18731E−20 | 5.64529E−18 |
| Hsph1 | −1.043326409 | 9.286330507 | 85.92665756 | 1.86734E−20 | 8.625E−18 |
| AW112010 | 0.444670626 | 11.45893149 | 85.76333436 | 2.02812E−20 | 9.10739E−18 |
| Rps12-ps3 | 0.611260131 | 9.758073988 | 83.06417161 | 7.94324E−20 | 3.47055E−17 |
| Rpl23a-ps3 | 0.404080492 | 10.53373269 | 82.08567098 | 1.30314E−19 | 5.51089E−17 |
| Cd160 | 1.385266485 | 9.322490009 | 82.0461108 | 1.32949E−19 | 5.51089E−17 |
| Fth1 | −0.334928954 | 11.36116368 | 81.30744771 | 1.93197E−19 | 7.80806E−17 |
| Rpl32 | 0.226778788 | 12.46778482 | 77.92038215 | 1.07279E−18 | 4.22992E−16 |
| Gm10073 | 0.679834085 | 9.542960393 | 77.36287093 | 1.42265E−18 | 5.47583E−16 |
| Gimap4 | 0.436236853 | 10.55167371 | 76.30539368 | 2.43019E−18 | 9.13639E−16 |
| Odc1 | −0.775525139 | 9.944056332 | 75.38533617 | 3.87255E−18 | 1.42281E−15 |
| Klf4 | −0.945293516 | 9.141545035 | 75.21282828 | 4.22612E−18 | 1.51821E−15 |
| Gm9493 | 0.455274074 | 10.19437568 | 74.04689103 | 7.62833E−18 | 2.68086E−15 |
| Epsti1 | 0.517090643 | 10.10953489 | 72.18893093 | 1.9555E−17 | 6.72607E−15 |
| Mat2a | −0.790901455 | 9.401350329 | 72.06029678 | 2.08721E−17 | 7.02956E−15 |
| Chchd2 | −0.376545479 | 10.32178394 | 71.90568348 | 2.25733E−17 | 7.44734E−15 |
| Zfp36l2 | 0.756650171 | 9.849977864 | 69.90504138 | 6.22292E−17 | 2.01199E−14 |
| Eef2 | 0.256530401 | 11.37716606 | 67.65949201 | 1.94315E−16 | 6.1594E−14 |
| Hspd1 | −0.730056082 | 9.56946388 | 67.51749258 | 2.08826E−16 | 6.36959E−14 |
| Tnfsf11 | −1.017394461 | 9.286194377 | 67.54703486 | 2.0572E−16 | 6.36959E−14 |
| Rpsa | 0.270139325 | 11.60964793 | 67.07276037 | 2.61669E−16 | 7.8336E−14 |
| Ubc | −0.40985659 | 10.95640452 | 66.95470801 | 2.77817E−16 | 8.1658E−14 |
| Limd2 | 0.51944246 | 10.09311916 | 66.50384614 | 3.49212E−16 | 1.0081E−13 |
| Ddx5 | −0.322175912 | 10.99872038 | 65.9875592 | 4.53778E−16 | 1.28698E−13 |
| Slc25a5 | 0.364261547 | 10.54681028 | 65.80084048 | 4.9887E−16 | 1.3669E−13 |
| Calm2 | −0.417904584 | 10.28145878 | 65.82454295 | 4.92906E−16 | 1.3669E−13 |
| Coro1a | 0.345777346 | 10.98317648 | 65.24019964 | 6.63031E−16 | 1.78643E−13 |
| Rpl7a-ps5 | 0.793213904 | 9.266019733 | 64.91202919 | 7.83175E−16 | 2.07554E−13 |
| Kdm6b | −0.838775113 | 9.554589167 | 64.82078504 | 8.20293E−16 | 2.13885E−13 |
| Cd7 | 1.097172739 | 9.645081853 | 64.25147561 | 1.0951E−15 | 2.81006E−13 |
| mt-Co1 | 0.234930964 | 12.34097755 | 61.19238243 | 5.17614E−15 | 1.30746E−12 |
| Gm11808 | 0.460625687 | 10.00121146 | 59.55269141 | 1.19063E−14 | 2.96119E−12 |
| Ptprcap | 0.468262667 | 10.14636839 | 59.11286349 | 1.48883E−14 | 3.64673E−12 |
| Rpl29 | 0.262184011 | 11.36451206 | 57.9475349 | 2.69203E−14 | 6.49542E−12 |
| Fam110a | −0.789509164 | 9.363253333 | 57.76260767 | 2.95738E−14 | 7.03075E−12 |
| Rpl39l | −0.660080135 | 9.110893413 | 56.68255512 | 5.12159E−14 | 1.19994E−11 |
| H2-K1 | −0.262139741 | 11.50207541 | 55.74456131 | 8.25263E−14 | 1.90589E−11 |
| Rpl15 | 0.305474683 | 10.64485513 | 55.5645479 | 9.044E−14 | 2.05923E−11 |
| Rpl13-ps3 | 0.582154842 | 9.53708088 | 55.37789667 | 9.94484E−14 | 2.23289E−11 |
| Rps6 | 0.187770999 | 12.12258817 | 54.28391249 | 1.73517E−13 | 3.84256E−11 |
| Klrk1 | −0.696235891 | 9.782659238 | 52.57186427 | 4.14789E−13 | 9.06146E−11 |
| Tnfsf8 | −0.869184457 | 9.70280662 | 52.09440691 | 5.28954E−13 | 1.13608E−10 |
| Lrrc58 | 0.663763744 | 9.389936985 | 52.07540605 | 5.34097E−13 | 1.13608E−10 |
| Wdr89 | 0.370232576 | 10.18931763 | 51.40555181 | 7.51258E−13 | 1.57725E−10 |
| Gpr18 | 0.866864527 | 9.411218906 | 51.09750716 | 8.78898E−13 | 1.82157E−10 |
| mt-Nd1 | −0.317004366 | 11.02202855 | 50.93349268 | 9.55492E−13 | 1.95525E−10 |
| Cdkn1a | −0.798684946 | 9.458316973 | 50.86367711 | 9.9009E−13 | 2.00072E−10 |
| Pycard | 0.551987428 | 9.777016154 | 49.94745009 | 1.57919E−12 | 3.15175E−10 |
| Rpl24 | 0.19603573 | 11.85276508 | 48.38011829 | 3.5111E−12 | 6.922E−10 |
| Rps26-ps1 | 0.687496784 | 9.283697606 | 47.81217608 | 4.6907E−12 | 9.13612E−10 |
| Lgals1 | −0.300166557 | 12.3861545 | 47.0816269 | 6.8091E−12 | 1.31043E−09 |
| Rpl18 | 0.192896256 | 11.84986773 | 46.75524775 | 8.04293E−12 | 1.52967E−09 |
| Tuba1b | −0.596743066 | 9.481069774 | 46.38393415 | 9.72093E−12 | 1.82731E−09 |
| Anxa1 | −0.789378766 | 9.401284199 | 45.91901426 | 1.23244E−11 | 2.26405E−09 |
| Eif3f | 0.262138365 | 10.76341364 | 45.93327111 | 1.2235E−11 | 2.26405E−09 |
| Arhgdib | 0.264978392 | 10.93540557 | 45.35015521 | 1.64775E−11 | 2.99298E−09 |
| S100a11 | −0.308609798 | 10.7547102 | 44.90204871 | 2.07141E−11 | 3.72071E−09 |
| Gm10250 | 0.669139793 | 9.283272179 | 44.69993982 | 2.29664E−11 | 4.07995E−09 |
| Actg1 | 0.251896211 | 12.51833862 | 44.01460144 | 3.25935E−11 | 5.72725E−09 |
| Rgs10 | 0.575935498 | 9.586443461 | 43.44995322 | 4.34942E−11 | 7.56051E−09 |
| Cox7a2l | 0.343579985 | 10.15410532 | 42.93720549 | 5.65254E−11 | 9.72116E−09 |
| Ctsw | 0.398042723 | 10.07562032 | 42.7050428 | 6.3648E−11 | 1.07181E−08 |
| Ifrd1 | −0.475489987 | 9.932972127 | 42.71437436 | 6.33451E−11 | 1.07181E−08 |
| Ccr5 | 0.70355753 | 9.447217476 | 42.05131782 | 8.89093E−11 | 1.48176E−08 |
| B2m | −0.150244995 | 12.75753363 | 41.50989787 | 1.17276E−10 | 1.93457E−08 |
| Gm10020 | 0.682288929 | 9.228632115 | 41.38530306 | 1.24994E−10 | 2.04106E−08 |
| Hspa5 | −0.33052293 | 10.94859063 | 41.32467722 | 1.28931E−10 | 2.0843E−08 |
| Cd44 | −0.53145037 | 9.521890103 | 40.78049304 | 1.70325E−10 | 2.72621E−08 |
| Id2 | −0.415453763 | 10.56572827 | 39.70676467 | 2.95102E−10 | 4.67707E−08 |
| Shisa5 | 0.241620621 | 10.99844694 | 39.41549416 | 3.42568E−10 | 5.37665E−08 |
| Srsf2 | −0.454044438 | 9.706632291 | 39.33527525 | 3.56934E−10 | 5.54827E−08 |
| Itgae | 0.822282028 | 9.255556378 | 39.14463452 | 3.93543E−10 | 6.05907E−08 |
| H2-Q7 | −0.287318486 | 10.53297959 | 38.93031587 | 4.39207E−10 | 6.63571E−08 |
| Nmrk1 | −0.649473302 | 9.332536123 | 38.93242527 | 4.38732E−10 | 6.63571E−08 |
| Gm10036 | 0.542587512 | 9.419981674 | 38.78703884 | 4.72656E−10 | 7.07496E−08 |
| Sh3bgrl3 | −0.213928684 | 11.44744765 | 38.64098235 | 5.09381E−10 | 7.55472E−08 |
| Txn1 | −0.357580971 | 10.03413913 | 38.5266841 | 5.40102E−10 | 7.93753E−08 |
| Ly6e | −0.295951849 | 10.68171292 | 37.98408352 | 7.13241E−10 | 1.03876E−07 |
| Pim1 | −0.383839128 | 10.72396533 | 37.73340148 | 8.11039E−10 | 1.17065E−07 |
| Rpl22l1 | 0.264217716 | 10.93780735 | 37.71481274 | 8.18804E−10 | 1.1714E−07 |

TABLE 3P
LPL-3 Ctrl vs. KO

| GeneSymbol | logFC | logCPM | LR | PValue | FDR |
|---|---|---|---|---|---|
| Dnajb1 | −1.946456328 | 9.715188532 | 322.0257087 | 5.244E−72 | 8.47744E−68 |
| Ubb | −0.556526755 | 12.64332085 | 264.5357982 | 1.76138E−59 | 1.42372E−55 |
| Hsp90ab1 | −0.842446585 | 11.04093619 | 262.9635073 | 3.87754E−59 | 2.08948E−55 |
| Malat1 | −0.657557225 | 14.44411562 | 252.3465366 | 7.99619E−57 | 3.23166E−53 |
| Dnaja1 | −0.967904935 | 10.3986654 | 203.5095769 | 3.58093E−46 | 1.15779E−42 |
| Hsp90aa1 | −1.016819062 | 10.47401553 | 201.0128142 | 1.2555E−45 | 3.38273E−42 |
| Hspa8 | −0.556803701 | 11.95258518 | 176.2104498 | 3.25747E−40 | 7.5229E−37 |
| Hspe1 | −0.95327844 | 10.18983826 | 153.0812553 | 3.67718E−35 | 7.43066E−32 |
| Hspa1a | −2.406354924 | 9.949094707 | 132.4560077 | 1.18913E−30 | 2.13594E−27 |
| Cxcr6 | 1.050964818 | 10.1716772 | 103.8800633 | 2.14941E−24 | 3.47474E−21 |
| Rpl27-ps3 | 0.677854872 | 9.862951638 | 103.2885245 | 2.89727E−24 | 4.25793E−21 |
| Hsph1 | −1.152569484 | 9.365951561 | 92.24800222 | 7.64673E−22 | 1.03014E−18 |
| Rpl9-ps6 | 0.616176975 | 9.871909672 | 83.51828779 | 6.31293E−20 | 7.85037E−17 |
| Fos | −1.398569273 | 9.446578559 | 82.92552785 | 8.52037E−20 | 9.83859E−17 |
| Gm2000 | 0.661448135 | 9.750013041 | 74.89725018 | 4.9586E−18 | 5.34405E−15 |
| Ifi27l2a | −0.89617847 | 10.07811554 | 73.7599711 | 8.82175E−18 | 8.91328E−15 |
| Wdr89 | 0.433531407 | 10.46020145 | 71.56018478 | 2.68929E−17 | 2.55736E−14 |
| Ddx5 | −0.359798857 | 11.12189179 | 71.1737819 | 3.27106E−17 | 2.93778E−14 |
| Gm11808 | 0.492106141 | 10.18664186 | 69.85758986 | 6.37444E−17 | 5.42364E−14 |
| Rpl6l | 0.439579948 | 10.33047525 | 69.08823159 | 9.41555E−17 | 7.61059E−14 |
| Ubald2 | −0.531794105 | 10.58366091 | 67.3611776 | 2.26057E−16 | 1.74021E−13 |
| Jund | −0.444613048 | 11.60111081 | 67.18787324 | 2.46828E−16 | 1.7956E−13 |
| Ccr2 | 0.982615192 | 9.627366071 | 67.12005026 | 2.55467E−16 | 1.7956E−13 |
| Rps27 | −0.22528728 | 13.41431811 | 64.64008117 | 8.99077E−16 | 6.05604E−13 |
| Eno1 | 0.56244217 | 9.973825903 | 64.44188359 | 9.94221E−16 | 6.42903E−13 |
| Hspd1 | −0.744360435 | 9.701312261 | 61.89149549 | 3.62914E−15 | 2.25648E−12 |
| Gm9843 | 0.399626408 | 10.54065217 | 61.2458297 | 5.03752E−15 | 3.01617E−12 |
| Gm8730 | 0.384178175 | 10.58492916 | 59.65981313 | 1.12756E−14 | 6.51003E−12 |
| Lgals1 | 0.584476207 | 11.42883276 | 59.05998416 | 1.52938E−14 | 8.5255E−12 |
| Gm10263 | 0.879988604 | 9.287197141 | 58.8792496 | 1.67651E−14 | 9.03415E−12 |
| S100a6 | 0.619339818 | 11.111098 | 55.07558334 | 1.15983E−13 | 6.04832E−11 |
| mt-Nd1 | −0.314291725 | 11.43254364 | 52.46700175 | 4.37539E−13 | 2.21039E−10 |
| Actn1 | −0.958369048 | 9.243454407 | 51.82637721 | 6.06318E−13 | 2.97022E−10 |
| mt-Nd2 | −0.461351834 | 10.07866988 | 51.42464038 | 7.43989E−13 | 3.53745E−10 |
| Cacybp | −0.705432725 | 9.458629507 | 51.23131446 | 8.20985E−13 | 3.68668E−10 |
| Arhgdib | 0.320566828 | 10.84726184 | 51.2464166 | 8.14693E−13 | 3.68668E−10 |
| Maff | −0.931067074 | 9.283544725 | 50.87231383 | 9.85743E−13 | 4.3069E−10 |
| H1f0 | 1.005362161 | 9.473187573 | 47.65841621 | 5.0734E−12 | 2.15833E−09 |
| Rpl7a-ps5 | 0.757646926 | 9.352518925 | 46.86769121 | 7.59444E−12 | 3.1156E−09 |
| Gm10036 | 0.598995698 | 9.558547095 | 46.83834526 | 7.70902E−12 | 3.1156E−09 |
| Fas | −0.830210368 | 9.383693055 | 46.49515393 | 9.18453E−12 | 3.62139E−09 |
| Bcl2 | −0.898899663 | 9.49783969 | 44.78359536 | 2.20059E−11 | 8.47016E−09 |
| Vim | 0.568575859 | 10.37415144 | 43.88211639 | 3.48761E−11 | 1.31118E−08 |
| Chchd2 | −0.336926527 | 10.38091917 | 43.82067836 | 3.59883E−11 | 1.32224E−08 |
| Gm6133 | 0.42291287 | 9.939660004 | 43.76172027 | 3.70889E−11 | 1.3324E−08 |
| Rpl10-ps3 | 0.525088242 | 9.670588182 | 42.35366951 | 7.61733E−11 | 2.67699E−08 |
| mt-Cytb | −0.260378871 | 11.31880065 | 42.11921612 | 8.58752E−11 | 2.95374E−08 |
| Gm10073 | 0.499465706 | 9.768923335 | 41.91880226 | 9.51433E−11 | 3.20435E−08 |
| Eif1 | −0.204726216 | 11.87265834 | 41.54646968 | 1.15102E−10 | 3.79743E−08 |
| Ass1 | 0.620830747 | 9.695025993 | 41.49478969 | 1.18185E−10 | 3.82117E−08 |
| Pfn1 | 0.254506795 | 12.11137348 | 41.2735189 | 1.3235E−10 | 4.19523E−08 |
| Fus | −0.445839928 | 9.904223746 | 40.96169319 | 1.55242E−10 | 4.82625E−08 |
| mt-Nd4 | −0.299211404 | 10.74171325 | 40.51332524 | 1.9528E−10 | 5.89125E−08 |
| Rpl13-ps3 | 0.498225143 | 9.711443418 | 40.49829211 | 1.96788E−10 | 5.89125E−08 |
| Rbm3 | 0.313163413 | 10.72414562 | 40.41601225 | 2.05252E−10 | 6.03292E−08 |
| Ltb4r1 | 0.861214833 | 9.337334137 | 39.74620311 | 2.89202E−10 | 8.33608E−08 |
| Tmem176b | 0.651358918 | 9.889767452 | 39.71457834 | 2.93923E−10 | 8.33608E−08 |
| Gm9844 | 0.538387087 | 9.610551657 | 39.1486532 | 3.92734E−10 | 1.09464E−07 |
| S100a4 | 0.676788049 | 10.14521787 | 39.10503468 | 4.01607E−10 | 1.1004E−07 |
| Furin | 0.614555215 | 10.39788225 | 38.37338333 | 5.84238E−10 | 1.57413E−07 |

TABLE 3Q
LPL-4 Ctrl vs. KO

| GeneSymbol | logFC | logCPM | LR | PValue | FDR |
|---|---|---|---|---|---|
| Hsp90ab1 | −1.377781478 | 11.67647139 | 543.8892971 | 2.6874E−120 | 4.3444E−116 |
| Dnajb1 | −2.622537269 | 11.47483313 | 463.2873417 | 9.2579E−103 | 7.4832E−99 |
| Hspa8 | −1.036886848 | 12.90024104 | 448.3195067 | 1.6743E−99 | 9.02236E−96 |
| Hsp90aa1 | −1.69852051 | 11.40941515 | 442.452405 | 3.16746E−98 | 1.28013E−94 |
| Hspa1a | −2.91446732 | 12.86010883 | 434.4143608 | 1.77875E−96 | 5.75104E−93 |
| Ubb | −0.8105054 | 13.50178123 | 308.2413372 | 5.27644E−69 | 1.42165E−65 |
| Dnaja1 | −1.298882131 | 11.23915542 | 290.2673755 | 4.34808E−65 | 1.00416E−61 |
| Hspa1b | −1.994309739 | 9.932282948 | 251.5650814 | 1.1837E−56 | 2.39196E−53 |
| Hspe1 | −1.211796243 | 10.68337716 | 228.4069201 | 1.3267E−51 | 2.38304E−48 |
| Hsph1 | −1.515688729 | 9.910103763 | 186.9056674 | 1.50586E−42 | 2.43438E−39 |
| Malat1 | −0.537789313 | 15.00626926 | 185.1910359 | 3.56528E−42 | 5.23966E−39 |
| Chchd2 | −0.665375364 | 10.5846311 | 154.6624546 | 1.65943E−35 | 2.23553E−32 |
| Cd160 | 1.665261637 | 9.620048846 | 144.2761916 | 3.09176E−33 | 3.84473E−30 |
| Phlda1 | −1.715829384 | 9.936081564 | 132.4571128 | 1.18847E−30 | 1.37234E−27 |
| Zfp36l2 | 1.020511228 | 10.14841765 | 130.7330703 | 2.83244E−30 | 3.05262E−27 |
| Hspd1 | −1.116030522 | 10.09787646 | 130.1171232 | 3.86298E−30 | 3.90306E−27 |
| Gm8730 | 0.618593594 | 10.48391426 | 128.1570741 | 1.03702E−29 | 9.86148E−27 |
| Gm9843 | 0.551372502 | 10.68584472 | 121.7644479 | 2.59938E−28 | 2.33453E−25 |
| Itgae | 1.320358 | 9.521589264 | 120.7193127 | 4.4021E−28 | 3.74549E−25 |
| Jund | −0.618823521 | 12.17587894 | 118.5549329 | 1.31073E−27 | 1.05947E−24 |
| Ccl5 | 1.166637714 | 13.51206478 | 116.9541133 | 2.93787E−27 | 2.2616E−24 |
| Ifrd1 | −0.930802696 | 10.22453595 | 115.1395806 | 7.33487E−27 | 5.38979E−24 |
| Rpl27-ps3 | 0.70516159 | 10.03539112 | 113.640381 | 1.5622E−26 | 1.09803E−23 |
| Rpl35 | 0.424975548 | 11.14942572 | 104.8025375 | 1.34934E−24 | 9.08896E−22 |
| Rpl23a-ps3 | 0.445117614 | 10.91295157 | 102.8250721 | 3.66087E−24 | 2.36726E−21 |
| Cacybp | −0.880317879 | 9.788879867 | 98.96384198 | 2.57154E−23 | 1.59891E−20 |
| Gzma | 1.764487784 | 10.77239663 | 96.82803577 | 7.56178E−23 | 4.52754E−20 |
| Rbm3 | 0.492723959 | 10.60453397 | 95.7412032 | 1.30927E−22 | 7.55916E−20 |
| Cdkn1a | −1.206995923 | 9.907033741 | 95.52859803 | 1.4577E−22 | 8.12594E−20 |
| Fosb | −1.066099038 | 9.880605281 | 90.88771259 | 1.52061E−21 | 8.19406E−19 |
| Rpl9-ps6 | 0.616227778 | 10.05860333 | 90.30945314 | 2.03677E−21 | 1.06214E−18 |
| Ubc | −0.604139188 | 11.71014404 | 87.02480623 | 1.07169E−20 | 5.41402E−18 |
| Odc1 | −0.946502247 | 10.10880077 | 85.58700716 | 2.21728E−20 | 1.0862E−17 |
| Tsc22d3 | 0.824896603 | 10.1820943 | 79.58469358 | 4.61991E−19 | 2.19663E−16 |
| Gm11808 | 0.505052668 | 10.40238007 | 79.36117782 | 5.17327E−19 | 2.38946E−16 |
| Wdr89 | 0.474528645 | 10.61488511 | 75.52093649 | 3.61553E−18 | 1.62357E−15 |
| Rpl6l | 0.488489759 | 10.34341037 | 74.33251242 | 6.60074E−18 | 2.88399E−15 |
| Atf3 | −1.301093577 | 9.677121904 | 73.33073331 | 1.09647E−17 | 4.66462E−15 |
| Bag3 | −1.266132157 | 9.521354891 | 71.1109823 | 3.37685E−17 | 1.39975E−14 |
| Cd7 | 1.383948283 | 9.71052912 | 66.63099706 | 3.27397E−16 | 1.32318E−13 |
| Ubald2 | −0.606943142 | 10.60239164 | 66.41228036 | 3.65816E−16 | 1.44239E−13 |
| Gm10073 | 0.60015302 | 9.897815633 | 65.33692218 | 6.31275E−16 | 2.42981E−13 |
| Dnaja4 | −1.047867988 | 9.592869946 | 64.97318745 | 7.59241E−16 | 2.85439E−13 |
| Rps27rt | 0.349506577 | 11.36556647 | 63.84509364 | 1.34597E−15 | 4.94522E−13 |
| Lmna | −1.039644505 | 9.877771656 | 63.70335194 | 1.44638E−15 | 5.19605E−13 |
| Eif3f | 0.348188622 | 10.67657857 | 59.9250672 | 9.85384E−15 | 3.46298E−12 |
| Gm6133 | 0.493613949 | 10.06524373 | 59.86826662 | 1.01424E−14 | 3.48854E−12 |
| Mat2a | −0.836955253 | 9.660876437 | 59.67147505 | 1.12089E−14 | 3.77508E−12 |
| Gm9493 | 0.435772602 | 10.45868098 | 59.06053171 | 1.52895E−14 | 5.0443E−12 |
| Tbc1d10c | 0.639902903 | 9.791392088 | 58.49880807 | 2.03414E−14 | 6.57679E−12 |
| Rps18-ps3 | 0.669636452 | 9.686630364 | 58.08106981 | 2.51535E−14 | 7.97317E−12 |
| Rpl32 | 0.21253747 | 12.37847718 | 57.65394918 | 3.12535E−14 | 9.71623E−12 |
| Gm2000 | 0.618749327 | 9.800115886 | 57.56436456 | 3.27098E−14 | 9.97712E−12 |
| H2-D1 | −0.216020929 | 12.2809488 | 55.65589398 | 8.63337E−14 | 2.58458E−11 |
| Rpl3 | 0.212911971 | 12.08878792 | 55.29303986 | 1.03836E−13 | 3.05201E−11 |
| Rpl13-ps3 | 0.557976851 | 9.854426518 | 54.57484183 | 1.49639E−13 | 4.30711E−11 |
| B2m | −0.176603891 | 12.90330204 | 54.54582092 | 1.51865E−13 | 4.30711E−11 |
| Rpl13a | 0.170081845 | 13.15924144 | 53.72717786 | 2.30355E−13 | 6.42054E−11 |
| Gm10036 | 0.640849878 | 9.721300594 | 53.68577585 | 2.3526E−13 | 6.44613E−11 |
| Rgs1 | 0.534698556 | 11.99808529 | 53.54098127 | 2.53254E−13 | 6.82352E−11 |
| Gm10076 | 0.596717543 | 9.779957709 | 53.44108257 | 2.66466E−13 | 7.06178E−11 |
| Gm10263 | 0.780604259 | 9.452513074 | 53.10791718 | 3.15717E−13 | 8.23208E−11 |
| Lcp1 | 0.396498657 | 10.52059347 | 52.01643536 | 5.5038E−13 | 1.41229E−10 |
| Hist1h1c | 0.957764201 | 9.701111152 | 51.90723574 | 5.81856E−13 | 1.46973E−10 |
| Ctla4 | −0.615817979 | 10.22784566 | 51.72555071 | 6.38267E−13 | 1.58742E−10 |
| Coq10b | −0.664020777 | 9.717601992 | 49.10934376 | 2.42084E−12 | 5.9296E−10 |
| Crem | −0.709446614 | 9.842415377 | 48.75660354 | 2.89781E−12 | 6.99195E−10 |
| Klf4 | −1.112826275 | 9.519315737 | 46.8956894 | 7.48672E−12 | 1.77986E−09 |
| mt-Nd1 | −0.32338555 | 10.96639023 | 46.25136642 | 1.04014E−11 | 2.43694E−09 |
| Ccdc117 | −0.779915449 | 9.506449603 | 44.82943889 | 2.14966E−11 | 4.96449E−09 |
| Rpl36-ps3 | 0.580626548 | 9.694858712 | 44.65567952 | 2.34915E−11 | 5.34879E−09 |
| Rgcc | −1.007094973 | 9.901750839 | 44.37211507 | 2.71527E−11 | 6.09654E−09 |
| Rpl10-ps3 | 0.562706811 | 9.75031317 | 44.05381348 | 3.19471E−11 | 7.07475E−09 |
| Rpl29 | 0.254087583 | 11.17785599 | 43.66475261 | 3.89728E−11 | 8.51398E−09 |
| 1810041H14Rik | 0.786738522 | 9.425523912 | 42.88865415 | 5.79457E−11 | 1.249E−08 |
| Eif2s3y | 0.700036231 | 9.470445177 | 41.39992401 | 1.24062E−10 | 2.6305E−08 |
| Rgs10 | 0.559350389 | 9.800303566 | 41.38062899 | 1.25293E−10 | 2.6305E−08 |
| Cxcr6 | 0.490736441 | 10.46808826 | 41.26892433 | 1.32661E−10 | 2.74949E−08 |
| Cxcl10 | −1.054785352 | 9.738782804 | 41.18958781 | 1.38157E−10 | 2.82714E−08 |
| Hspa5 | −0.366763461 | 10.83492414 | 39.82661265 | 2.77538E−10 | 5.60834E−08 |
| Apobec3 | 0.575215862 | 9.721277042 | 39.53476829 | 3.2227E−10 | 6.43188E−08 |
| Slfn2 | 0.468248293 | 10.13448256 | 39.34340969 | 3.55451E−10 | 7.00758E−08 |
| Dedd2 | −0.668986564 | 9.545498301 | 38.99006944 | 4.25967E−10 | 8.2966E−08 |
| Fth1 | −0.247812637 | 11.54524749 | 38.79281379 | 4.7126E−10 | 9.0695E−08 |
| Ptprcap | 0.436049079 | 10.09758212 | 38.75789964 | 4.79765E−10 | 9.12456E−08 |
| H2-K1 | −0.215473813 | 11.62297555 | 38.21117955 | 6.34876E−10 | 1.19342E−07 |

TABLE 3R
LPL-5 Ctrl vs. KO

| GeneSymbol | logFC | logCPM | LR | PValue | FDR |
|---|---|---|---|---|---|
| Ubb | −0.667747794 | 13.05670984 | 265.7156028 | 9.74321E−60 | 1.57509E−55 |
| Hsp90aa1 | −1.390021311 | 10.77771699 | 236.9250263 | 1.84164E−53 | 1.4886E−49 |
| Hsp90ab1 | −0.877553386 | 11.22553208 | 214.086737 | 1.76318E−48 | 9.50116E−45 |
| Dnajb1 | −2.034720283 | 10.05660873 | 212.4063054 | 4.10102E−48 | 1.65743E−44 |
| Gm8730 | 0.851933726 | 10.24578436 | 206.9946236 | 6.21693E−47 | 2.01006E−43 |
| Hspa1a | −3.201994123 | 10.61491682 | 204.4379641 | 2.24606E−46 | 6.05162E−43 |
| Hspa8 | −0.790773697 | 12.33316572 | 185.8795749 | 2.52221E−42 | 5.82487E−39 |
| Dnaja1 | −1.115058706 | 10.84366253 | 184.3752183 | 5.37272E−42 | 1.08569E−38 |
| Rpl35 | 0.583624905 | 10.97458616 | 157.7700054 | 3.47453E−36 | 6.24103E−33 |
| Hspe1 | −1.122714984 | 10.26957624 | 131.5655283 | 1.86225E−30 | 3.01051E−27 |
| Malat1 | −0.529186268 | 14.79984657 | 126.899325 | 1.95441E−29 | 2.87227E−26 |
| Gm9843 | 0.631738591 | 10.43647932 | 122.2523516 | 2.03268E−28 | 2.73835E−25 |
| Rpl9-ps6 | 0.809660987 | 9.759551999 | 106.7457447 | 5.06108E−25 | 6.29365E−22 |
| Rpl6l | 0.633388877 | 10.12675606 | 100.075177 | 1.46721E−23 | 1.69421E−20 |
| Gm9493 | 0.571951632 | 10.23323023 | 85.51740481 | 2.29671E−20 | 2.47524E−17 |
| Rpl27-ps3 | 0.729614246 | 9.781981089 | 84.53046761 | 3.78339E−20 | 3.82265E−17 |
| Rpl23a-ps3 | 0.473928723 | 10.60530193 | 83.90585402 | 5.18906E−20 | 4.9345E−17 |
| Hsph1 | −1.177413258 | 9.464086511 | 74.92950145 | 4.87826E−18 | 4.38122E−15 |
| Gm11808 | 0.601111889 | 10.07554584 | 72.80397873 | 1.43188E−17 | 1.2183E−14 |
| Gm2000 | 0.802070822 | 9.596573833 | 70.33377085 | 5.00731E−17 | 4.04741E−14 |
| Hspd1 | −0.890332867 | 9.826990882 | 67.99041097 | 1.64293E−16 | 1.26474E−13 |
| Rpl32 | 0.263487778 | 12.19599702 | 64.29385621 | 1.07179E−15 | 7.87574E−13 |
| Satb1 | 0.819131319 | 9.737592769 | 63.08567762 | 1.97908E−15 | 1.39103E−12 |
| Rplp0 | 0.258058129 | 12.11340747 | 60.06163932 | 9.19327E−15 | 6.19244E−12 |
| Ddx5 | −0.338656579 | 11.43119638 | 58.94745616 | 1.61939E−14 | 1.04716E−11 |
| Rpl24 | 0.266833972 | 11.64961557 | 58.38416317 | 2.1562E−14 | 1.34066E−11 |
| Fosb | −1.074649073 | 9.490138093 | 57.46868713 | 3.43403E−14 | 2.05609E−11 |
| Rps12-ps3 | 0.587568991 | 9.824084704 | 55.69125561 | 8.47946E−14 | 4.89568E−11 |
| Rbm3 | 0.389737471 | 10.63055567 | 55.06584393 | 1.16559E−13 | 6.49756E−11 |
| Rpl10-ps3 | 0.711450504 | 9.547172531 | 54.99892102 | 1.20596E−13 | 6.49852E−11 |
| Gm10073 | 0.644880368 | 9.623688778 | 52.69248962 | 3.9008E−13 | 2.0342E−10 |
| Jund | −0.397700454 | 12.17425697 | 52.22636503 | 4.94576E−13 | 2.49854E−10 |
| Prr7 | −0.639601864 | 10.04331603 | 52.06546063 | 5.36809E−13 | 2.62971E−10 |
| Itgae | 1.123657217 | 9.319838841 | 51.56974696 | 6.90982E−13 | 3.28542E−10 |
| Eef2 | 0.29305387 | 11.17127454 | 50.2993106 | 1.31996E−12 | 6.05646E−10 |
| Hspa1b | −1.112341319 | 9.284322097 | 50.25702435 | 1.34871E−12 | 6.05646E−10 |
| Wdr89 | 0.439221506 | 10.3352908 | 49.53293438 | 1.95069E−12 | 8.52295E−10 |
| Gm6133 | 0.523936873 | 9.843311357 | 47.94692977 | 4.37913E−12 | 1.86298E−09 |
| Gm10263 | 0.852181403 | 9.264949602 | 47.75742561 | 4.82354E−12 | 1.99942E−09 |
| Chchd2 | −0.36297706 | 10.50692552 | 47.31500857 | 6.04476E−12 | 2.44299E−09 |
| Cd160 | 1.185837125 | 9.397166234 | 45.0097638 | 1.96054E−11 | 7.73028E−09 |
| AY036118 | 0.801085044 | 9.298633911 | 44.02645037 | 3.23968E−11 | 1.24697E−08 |
| Rpl29 | 0.291653565 | 10.96053826 | 43.6349489 | 3.95709E−11 | 1.48768E−08 |
| Rpsa | 0.268788369 | 11.41599406 | 43.26500152 | 4.78058E−11 | 1.75643E−08 |
| Rps18-ps3 | 0.697609539 | 9.437736028 | 42.05344044 | 8.88128E−11 | 3.19055E−08 |
| Rpl13-ps3 | 0.566952616 | 9.614334098 | 40.26690495 | 2.2153E−10 | 7.78534E−08 |
| Gm10076 | 0.592960428 | 9.574070265 | 39.75217493 | 2.88319E−10 | 9.91696E−08 |
| Uba52 | 0.418146824 | 10.13486888 | 39.60024883 | 3.11644E−10 | 1.04959E−07 |
| Rps27rt | 0.320302058 | 10.99650998 | 39.32759785 | 3.58341E−10 | 1.18223E−07 |
| Rps26-ps1 | 0.70530421 | 9.384930749 | 38.94602666 | 4.35686E−10 | 1.38104E−07 |
| Rpl15 | 0.323801677 | 10.62581306 | 38.96325972 | 4.31857E−10 | 1.38104E−07 |
| mt-Co1 | 0.23982267 | 12.12069306 | 38.69051414 | 4.96617E−10 | 1.5439E−07 |
| Rps11 | 0.186996755 | 12.06257295 | 37.34887398 | 9.87779E−10 | 3.01291E−07 |

TABLE 3S
LPL-6 Ctrl vs. KO

| GeneSymbol | logFC | logCPM | LR | PValue | FDR |
|---|---|---|---|---|---|
| Rpl27-ps3 | 1.000892179 | 9.677930454 | 232.0702931 | 2.10792E−52 | 3.40767E−48 |
| Hsp90aa1 | −1.210351983 | 10.47955889 | 202.9526524 | 4.73718E−46 | 3.60335E−42 |
| Gm8730 | 0.756647108 | 10.38713922 | 202.2665877 | 6.68691E−46 | 3.60335E−42 |
| Hspa1a | −3.243435226 | 9.933983278 | 198.1462725 | 5.3011E−45 | 2.14244E−41 |
| Ubb | −0.574565023 | 12.67292372 | 180.0597604 | 4.70297E−41 | 1.52056E−37 |
| Dnajb1 | −1.702734682 | 9.424438564 | 179.4808902 | 6.29164E−41 | 1.69518E−37 |
| Wdr89 | 0.640598855 | 10.29329469 | 176.5192226 | 2.78904E−40 | 6.4411E−37 |
| Gm9843 | 0.665821745 | 10.37262602 | 158.7124543 | 2.16256E−36 | 4.37E−33 |
| Rpl9-ps6 | 0.849063028 | 9.614175562 | 142.8508334 | 6.33644E−33 | 1.13817E−29 |
| Rpl6l | 0.667371325 | 10.05028615 | 139.6822252 | 3.12395E−32 | 5.05018E−29 |
| Dnaja1 | −0.936847633 | 10.37482583 | 137.9055231 | 7.64278E−32 | 1.12321E−28 |
| Rpl35 | 0.474243562 | 11.1600212 | 133.1346392 | 8.44835E−31 | 1.13813E−27 |
| Gm2000 | 0.900801116 | 9.411532299 | 131.3566669 | 2.06886E−30 | 2.5727E−27 |
| Hsp90ab1 | −0.638919781 | 11.00174478 | 119.0868662 | 1.00242E−27 | 1.15751E−24 |
| Malat1 | −0.541217017 | 14.12310599 | 110.177485 | 8.95985E−26 | 9.65633E−23 |
| Rpl23a-ps3 | 0.532413845 | 10.61742407 | 109.6556439 | 1.16582E−25 | 1.17791E−22 |
| Gm11808 | 0.647313204 | 9.982489755 | 106.0637432 | 7.14011E−25 | 6.78983E−22 |
| Rpl10-ps3 | 0.93087986 | 9.304624268 | 102.2688908 | 4.84745E−24 | 4.35355E−21 |
| Gm9493 | 0.557945752 | 10.19580436 | 100.6832828 | 1.07933E−23 | 9.18337E−21 |
| Gm10073 | 0.803166764 | 9.440142111 | 94.98063058 | 1.92257E−22 | 1.55402E−19 |
| Hspe1 | −0.787573629 | 10.02870964 | 81.44687154 | 1.80037E−19 | 1.38594E−16 |
| Rps18-ps3 | 0.874430698 | 9.141091849 | 81.14090097 | 2.10185E−19 | 1.54447E−16 |
| Gm6133 | 0.609584976 | 9.693751155 | 79.56643779 | 4.66279E−19 | 3.27734E−16 |
| Gm10076 | 0.78780773 | 9.320701955 | 76.68494977 | 2.00526E−18 | 1.35071E−15 |
| Gm10036 | 0.810805014 | 9.213962076 | 74.61076882 | 5.73296E−18 | 3.70716E−15 |
| Rpl7a-ps5 | 0.908100854 | 9.003418976 | 71.62566534 | 2.60151E−17 | 1.61754E−14 |
| Gm9844 | 0.795707711 | 9.253843776 | 70.30676693 | 5.07633E−17 | 3.0394E−14 |
| Rpl36-ps3 | 0.826670812 | 9.144263881 | 68.68823221 | 1.15327E−16 | 6.65849E−14 |
| AY036118 | 1.000399497 | 8.8526315 | 58.45878653 | 2.07594E−14 | 1.11866E−11 |
| Lrrc58 | 0.828432135 | 9.10202243 | 58.49410236 | 2.03901E−14 | 1.11866E−11 |
| Gm10250 | 0.841380514 | 8.998216795 | 56.86543552 | 4.66677E−14 | 2.43364E−11 |
| Rpl15 | 0.321012912 | 10.70053263 | 55.03639559 | 1.18318E−13 | 5.9773E−11 |
| Lmna | −1.033273941 | 9.199192092 | 53.22605295 | 2.9729E−13 | 1.45636E−10 |
| Rps12-ps3 | 0.511506106 | 9.706346193 | 53.00742095 | 3.32291E−13 | 1.57994E−10 |
| Uba52 | 0.452683276 | 10.01489978 | 51.85402704 | 5.97839E−13 | 2.76133E−10 |
| Rpl13-ps3 | 0.621729495 | 9.346679461 | 48.71691561 | 2.95705E−12 | 1.32788E−09 |
| Rpl29 | 0.258694868 | 11.28929882 | 46.03406713 | 1.16214E−11 | 5.07763E−09 |
| Jund | −0.427242629 | 11.18646997 | 44.56718827 | 2.45776E−11 | 1.04558E−08 |
| Junb | −0.438372159 | 11.38076207 | 43.97788044 | 3.32108E−11 | 1.37663E−08 |
| Gm42418 | 0.725684143 | 8.981321303 | 42.89367766 | 5.77971E−11 | 2.33587E−08 |
| Rps27rt | 0.353162983 | 10.92491058 | 39.91812858 | 2.64834E−10 | 1.04422E−07 |
| Chchd2 | −0.311772562 | 10.31406676 | 39.44480899 | 3.37464E−10 | 1.29891E−07 |
| Rbm3 | 0.32207408 | 10.71438018 | 38.81533478 | 4.65854E−10 | 1.71628E−07 |
| Rps26-ps1 | 0.718056862 | 9.019003364 | 38.80999724 | 4.67129E−10 | 1.71628E−07 |
| Ddx5 | −0.278755543 | 10.97294105 | 37.85862663 | 7.60613E−10 | 2.73246E−07 |
| Cdkn1a | −0.79324254 | 9.191708702 | 37.79083713 | 7.87508E−10 | 2.76758E−07 |

TABLE 3T
LPL-7 Ctrl vs. KO

| GeneSymbol | logFC | logCPM | LR | PValue | FDR |
|---|---|---|---|---|---|
| Gm9843 | 0.679076895 | 10.68936848 | 85.64667834 | 2.15137E−20 | 2.46863E−16 |
| Wdr89 | 0.741913181 | 10.65124957 | 84.95385849 | 3.0541E−20 | 2.46863E−16 |
| Hspa8 | −0.660530188 | 12.56155791 | 81.61422367 | 1.65419E−19 | 8.9139E−16 |
| Dnajb1 | −1.649042898 | 10.57015052 | 80.42659398 | 3.01707E−19 | 1.12709E−15 |
| Hsp90ab1 | −0.784877451 | 11.53679216 | 80.14113929 | 3.48598E−19 | 1.12709E−15 |
| Rpl27-ps3 | 0.886089505 | 10.09707719 | 79.75671931 | 4.23468E−19 | 1.14096E−15 |
| Ubb | −0.498645077 | 13.14137294 | 79.18808927 | 5.64695E−19 | 1.30412E−15 |
| Hsp90aa1 | −1.001997476 | 11.1674382 | 72.06971016 | 2.07728E−17 | 4.19766E−14 |
| Rps27rt | 0.538171151 | 11.50969557 | 70.70374503 | 4.15104E−17 | 7.45619E−14 |
| Dnaja1 | −0.843376752 | 11.10860762 | 62.44563998 | 2.73902E−15 | 4.42789E−12 |
| Rpl35 | 0.489450987 | 11.11111461 | 58.91362524 | 1.64747E−14 | 2.42119E−11 |
| Malat1 | −0.461372669 | 14.92882516 | 58.52275262 | 2.00954E−14 | 2.70718E−11 |
| Gm8730 | 0.632965858 | 10.46529795 | 56.55413113 | 5.46724E−14 | 6.79872E−11 |
| Rpl9-ps6 | 0.735601623 | 10.06584632 | 49.89813247 | 1.61939E−12 | 1.86993E−09 |
| Rps18-ps3 | 0.949329603 | 9.757081417 | 49.11636357 | 2.41219E−12 | 2.5997E−09 |
| Rpl23a-ps3 | 0.516177035 | 10.81532166 | 45.89587466 | 1.24708E−11 | 1.26002E−08 |
| Gm11808 | 0.599716998 | 10.3153324 | 44.88415961 | 2.09042E−11 | 1.98787E−08 |
| Rpl6l | 0.570669176 | 10.35956478 | 44.21034092 | 2.94919E−11 | 2.6487E−08 |
| Hspe1 | −0.857543595 | 10.72567371 | 43.97285148 | 3.32962E−11 | 2.83298E−08 |
| Zfp36l2 | 0.848268603 | 10.19106218 | 43.66380014 | 3.89918E−11 | 3.15171E−08 |
| Rps12-ps3 | 0.693919785 | 10.07841838 | 42.55899212 | 6.85823E−11 | 5.27953E−08 |
| Rpl13a | 0.252632783 | 13.2005704 | 40.88104103 | 1.61783E−10 | 1.18881E−07 |
| Rpl13-ps3 | 0.768495136 | 9.869026408 | 38.6191581 | 5.15108E−10 | 3.62054E−07 |

TABLE 3U
LPL-8 Ctrl vs. KO

| GeneSymbol | logFC | logCPM | LR | PValue | FDR |
|---|---|---|---|---|---|
| Hspa1a | −4.46133106 | 9.487194307 | 303.1093704 | 6.92398E−68 | 1.11933E−63 |
| Hsp90aa1 | −1.392806426 | 10.0823126 | 226.1411471 | 4.13928E−51 | 3.34578E−47 |
| Dnajb1 | −1.900971523 | 8.769887068 | 179.91552 | 5.05668E−41 | 2.72488E−37 |
| Rbm3 | 0.641674082 | 11.24281554 | 156.1928534 | 7.6829E−36 | 3.10504E−32 |
| Gm8730 | 0.780025906 | 10.21672865 | 140.0001899 | 2.66178E−32 | 8.60607E−29 |
| Hspa8 | −0.674361493 | 11.79442321 | 131.7442211 | 1.70194E−30 | 4.58559E−27 |
| Hspa1b | −2.527753243 | 8.164889167 | 119.0070748 | 1.04357E−27 | 2.41004E−24 |
| Malat1 | −0.913828296 | 12.8841938 | 103.0583966 | 3.25413E−24 | 6.48903E−21 |
| Hsp90ab1 | −0.711121034 | 10.61566746 | 102.851369 | 3.6126E−24 | 6.48903E−21 |
| Ubb | −0.618061035 | 12.11138082 | 88.5469168 | 4.96434E−21 | 8.02535E−18 |
| Rplp0 | 0.386720187 | 12.56982724 | 80.78667159 | 2.51448E−19 | 3.69537E−16 |
| Hsph1 | −1.509444326 | 8.280317907 | 75.08869481 | 4.50036E−18 | 6.06273E−15 |
| Hspe1 | −0.879787784 | 9.328717894 | 71.0337361 | 3.51169E−17 | 4.36692E−14 |
| Ubc | −0.738216636 | 10.18004219 | 70.86468308 | 3.82586E−17 | 4.41777E−14 |
| Rpl35 | 0.450528289 | 11.14097897 | 68.69089929 | 1.15171E−16 | 1.24124E−13 |
| Dnaja1 | −0.660080702 | 9.700853705 | 57.96742407 | 2.66495E−14 | 2.69259E−11 |
| Rpl27-ps3 | 0.769290147 | 8.860371085 | 56.31921573 | 6.16104E−14 | 5.85878E−11 |
| Gm2000 | 0.803158017 | 8.865313337 | 51.98860839 | 5.58236E−13 | 5.01358E−10 |
| Gm10076 | 0.792181373 | 8.938297761 | 49.98552982 | 1.54884E−12 | 1.31782E−09 |
| H2-K1 | −0.515701257 | 10.79228433 | 47.79738871 | 4.72621E−12 | 3.8202E−09 |
| H2-Q7 | −0.62224609 | 9.790270068 | 47.25801338 | 6.2231E−12 | 4.79061E−09 |
| Rpl3 | 0.295505002 | 11.93101795 | 45.77011002 | 1.32977E−11 | 9.7714E−09 |
| Gm10073 | 0.771973453 | 8.899753731 | 44.51396459 | 2.52549E−11 | 1.77509E−08 |
| Rpl10-ps3 | 0.794752736 | 8.799079764 | 43.51094877 | 4.21594E−11 | 2.83979E−08 |
| Rpl13 | 0.295566129 | 12.2801827 | 43.04061266 | 5.36153E−11 | 3.46698E−08 |
| Rpl32 | 0.300115748 | 12.37635341 | 42.59945259 | 6.71782E−11 | 4.17693E−08 |
| Jund | −0.551080322 | 10.78767876 | 42.38224753 | 7.50683E−11 | 4.49465E−08 |
| Rpl10a | 0.389587396 | 11.15066686 | 41.45258432 | 1.20765E−10 | 6.73201E−08 |
| Isg15 | −1.276987474 | 8.306648902 | 41.51123525 | 1.17195E−10 | 6.73201E−08 |
| Rps18-ps3 | 0.999323395 | 8.484337215 | 40.88283659 | 1.61634E−10 | 8.70994E−08 |
| Fosb | −1.399459966 | 8.173149877 | 40.59851305 | 1.86949E−10 | 9.74908E−08 |
| Rps18 | 0.315086971 | 12.25780987 | 40.12474383 | 2.38252E−10 | 1.17306E−07 |
| Rpl18a | 0.239964236 | 12.26488225 | 40.11487131 | 2.39459E−10 | 1.17306E−07 |
| Rpl18 | 0.269829889 | 11.37724478 | 39.31803433 | 3.601E−10 | 1.71217E−07 |
| Rps4x | 0.281160834 | 11.75181977 | 39.26100381 | 3.70773E−10 | 1.71255E−07 |

TABLE 3V
LPL-9 Ctrl vs. KO

| GeneSymbol | logFC | logCPM | LR | PValue | FDR |
|---|---|---|---|---|---|
| Hspa1a | −3.208006096 | 10.63021086 | 97.57359097 | 5.18914E−23 | 8.38877E−19 |
| Ubb | −0.760618634 | 12.38390677 | 60.1939278 | 8.5957E−15 | 6.9479E−11 |
| Gzma | 2.923731333 | 12.03426076 | 56.95387224 | 4.46155E−14 | 2.40418E−10 |
| Hspa1b | −2.544460073 | 9.795567163 | 53.74015844 | 2.28838E−13 | 9.24847E−10 |
| Hsp90aa1 | −1.50442418 | 10.20067248 | 42.7113425 | 6.34433E−11 | 2.05125E−07 |
| Ccr9 | 2.221991151 | 9.9641527 | 40.55436946 | 1.9122E−10 | 5.15212E−07 |

TABLE 3W
LPL-10 Ctrl vs. KO

| GeneSymbol | logFC | logCPM | LR | PValue | FDR |
|---|---|---|---|---|---|
| Hspa1a | −3.279080597 | 9.77749525 | 66.6687553 | 3.21186E−16 | 5.19229E−12 |
| Malat1 | −0.906756376 | 13.82786584 | 48.20036621 | 3.84816E−12 | 3.11047E−08 |
| Dnajb1 | −1.9736233 | 9.859710517 | 47.29375921 | 6.11065E−12 | 3.29282E−08 |
| Ubb | −0.62876509 | 12.23504229 | 27.75960127 | 1.37365E−07 | 0.00055516 |
| Dnaja1 | −1.070648437 | 10.15612377 | 21.95233617 | 2.79506E−06 | 0.009037003 |

TABLE 3X
LPL-11 Ctrl vs. KO

| GeneSymbol | logFC | logCPM | LR | PValue | FDR |
|---|---|---|---|---|---|
| Gm9843 | 1.152747768 | 10.86191498 | 107.7332038 | 3.07507E−25 | 4.97117E−21 |
| Rps27rt | 0.936042362 | 11.6006599 | 98.51241281 | 3.22994E−23 | 2.61076E−19 |
| Uba52 | 1.259153747 | 10.29857567 | 89.76924229 | 2.67623E−21 | 1.25309E−17 |
| Gm11808 | 1.174254179 | 10.36510957 | 89.13644101 | 3.685E−21 | 1.25309E−17 |
| Rpl6l | 1.12148656 | 10.43521049 | 89.03662136 | 3.87571E−21 | 1.25309E−17 |
| Gm8730 | 1.178269708 | 10.48527954 | 88.52016702 | 5.03192E−21 | 1.35577E−17 |
| Rpl23a-ps3 | 0.97111709 | 11.05711726 | 87.23097408 | 9.65597E−21 | 2.22998E−17 |
| Rpl9-ps6 | 1.361164957 | 10.11088474 | 85.89170087 | 1.90065E−20 | 3.41399E−17 |
| Gm9493 | 1.04400959 | 10.54876454 | 85.92861245 | 1.8655E−20 | 3.41399E−17 |
| Rpl27-ps3 | 1.236714099 | 10.05185113 | 73.33936538 | 1.09169E−17 | 1.76482E−14 |
| Gm2000 | 1.283533859 | 9.738432617 | 68.91660374 | 1.02716E−16 | 1.50956E−13 |
| Rpl35 | 0.739766449 | 11.21870405 | 65.61762991 | 5.47468E−16 | 7.37531E−13 |
| Gm10036 | 1.489161877 | 9.448810832 | 64.09537827 | 1.18539E−15 | 1.47408E−12 |
| Wdr89 | 0.805998267 | 10.97502014 | 59.64331287 | 1.13705E−14 | 1.31297E−11 |
| Gm10073 | 1.302016708 | 9.701129124 | 56.54648834 | 5.48853E−14 | 5.91517E−11 |
| Gm10076 | 1.506003095 | 9.391444214 | 50.19653071 | 1.39094E−12 | 1.40537E−09 |
| Rpl15 | 0.715629478 | 10.88701252 | 45.13720707 | 1.83702E−11 | 1.7469E−08 |
| Rpl36-ps3 | 1.287158449 | 9.442853364 | 41.85453352 | 9.83225E−11 | 8.83046E−08 |
| Gm6133 | 0.942044122 | 9.994215819 | 40.6518122 | 1.81918E−10 | 1.54784E−07 |

TABLE 3Y
GWAS

| GeneSymbol | LPL-2 logFC | LPL-2 FDR | LPL-9 logFC | LPL-9 FDR | LPL-7 logFC | LPL-7 FDR | GWAS? |
|---|---|---|---|---|---|---|---|
| Zfp36l2 | 0.757 | 2.01E−14 | 1.283 | 6.35E−03 | 0.848 | 3.15E−08 | TRUE |
| Tnfsf8 | −0.869 | 1.14E−10 | −0.918 | 4.04E−01 | −0.359 | 9.46E−01 | TRUE |
| Gpr18 | 0.867 | 1.82E−10 | 1.208 | 8.21E−02 | 0.102 | 1.00E+00 | TRUE |
| Ctsw | 0.398 | 1.07E−08 | 0.231 | 1.00E+00 | 0.379 | 2.95E−01 | TRUE |
| Ccr5 | 0.704 | 1.48E−08 | 0.294 | 1.00E+00 | 0.239 | 7.62E−01 | TRUE |
| Krtcap2 | −0.312 | 3.46E−07 | −0.676 | 5.81E−03 | −0.003 | 1.00E+00 | TRUE |
| Rpl3 | 0.149 | 2.99E−06 | 0.169 | 9.15E−01 | 0.195 | 2.69E−03 | TRUE |
| Crem | −0.459 | 3.59E−05 | −0.286 | 1.00E+00 | −0.580 | 2.86E−02 | TRUE |
| Cd28 | −0.289 | 5.47E−05 | 0.156 | 1.00E+00 | 0.237 | 3.59E−01 | TRUE |
| Lsp1 | 0.201 | 7.10E−04 | −0.014 | 1.00E+00 | 0.098 | 1.00E+00 | TRUE |
| Il7r | 0.351 | 7.68E−04 | 0.540 | 8.46E−01 | 0.910 | 8.48E−04 | TRUE |
| Irf1 | −0.433 | 1.05E−03 | 0.143 | 1.00E+00 | −0.190 | 1.00E+00 | TRUE |
| Itga4 | 0.408 | 2.71E−03 | 0.595 | 9.15E−01 | 0.030 | 1.00E+00 | TRUE |
| Apobr | −0.325 | 7.72E−03 | −0.049 | 1.00E+00 | 0.009 | 1.00E+00 | TRUE |
| Tnfrsf9 | −0.127 | 9.01E−01 | −0.515 | 9.15E−01 | −0.836 | 8.81E−03 | TRUE |
| Ccnd3 | −0.283 | 1.64E−02 | −0.302 | 1.00E+00 | 0.212 | 9.60E−01 | TRUE |
| Csf2 | −0.426 | 1.96E−02 | −0.124 | 1.00E+00 | 0.000 | 1.00E+00 | TRUE |
| Fermt3 | 0.314 | 2.58E−02 | 0.043 | 1.00E+00 | 0.117 | 1.00E+00 | TRUE |
| Dpm3 | −0.287 | 3.08E−02 | 0.124 | 1.00E+00 | 0.129 | 1.00E+00 | TRUE |
| Gnb2 | 0.204 | 3.37E−02 | −0.279 | 1.00E+00 | 0.375 | 7.83E−02 | TRUE |
| Dap | 0.340 | 3.38E−02 | 0.719 | 7.71E−01 | −0.084 | 1.00E+00 | TRUE |
| Cd74 | −0.489 | 3.79E−02 | −0.385 | 1.00E+00 | −0.539 | 3.37E−01 | TRUE |
| Fos | −0.534 | 4.09E−02 | −0.878 | 8.71E−01 | −0.802 | 3.89E−02 | TRUE |
| St13 | −0.293 | 4.51E−02 | 0.536 | 9.15E−01 | −0.079 | 1.00E+00 | TRUE |
| Fam213b | −0.144 | 5.40E−02 | 0.000 | 1.00E+00 | 0.051 | 1.00E+00 | TRUE |
| Cd6 | 0.228 | 5.53E−02 | −0.222 | 1.00E+00 | −0.067 | 1.00E+00 | TRUE |
| Dnajb4 | −0.343 | 6.08E−02 | −0.126 | 1.00E+00 | −0.059 | 1.00E+00 | TRUE |
| Ptprc | 0.165 | 6.25E−02 | 0.265 | 1.00E+00 | −0.053 | 1.00E+00 | TRUE |
| Socs1 | −0.338 | 6.39E−02 | −0.358 | 1.00E+00 | −0.233 | 9.77E−01 | TRUE |
| Ncf4 | 0.292 | 6.51E−02 | −0.207 | 1.00E+00 | 0.381 | 5.41E−01 | TRUE |
| Rps14 | −0.004 | 1.00E+00 | −0.003 | 1.00E+00 | 0.138 | 6.58E−02 | TRUE |
| Vmp1 | 0.273 | 6.92E−02 | 0.818 | 6.99E−01 | 0.202 | 9.46E−01 | TRUE |
| Cd226 | 0.350 | 7.33E−02 | 0.189 | 1.00E+00 | 0.086 | 1.00E+00 | TRUE |
| Hdac7 | 0.285 | 7.88E−02 | 0.174 | 1.00E+00 | 0.124 | 1.00E+00 | TRUE |
| Pim3 | −0.293 | 2.16E−01 | −0.032 | 1.00E+00 | −0.652 | 8.14E−02 | TRUE |
| Traf3ip2 | 0.286 | 9.01E−02 | 0.462 | 7.75E−01 | −0.003 | 1.00E+00 | TRUE |
| Ets1 | 0.162 | 9.20E−02 | −0.032 | 1.00E+00 | 0.108 | 9.68E−01 | TRUE |
| Lpxn | 0.211 | 9.69E−02 | 0.245 | 1.00E+00 | 0.258 | 7.35E−01 | TRUE |
| Dnajb1 | −2.545 | 7.19E−117 | −1.766 | 3.18E−06 | −1.649 | 1.13E−15 | FALSE |
| Ubb | −0.719 | 6.98E−110 | −0.761 | 6.95E−11 | −0.499 | 1.30E−15 | FALSE |
| Hspa1a | −4.265 | 1.73E−105 | −3.208 | 8.39E−19 | −1.733 | 5.26E−06 | FALSE |
| Hsp90aa1 | −1.539 | 9.57E−97 | −1.504 | 2.05E−07 | −1.002 | 4.20E−14 | FALSE |
| Hsp90ab1 | −1.045 | 1.54E−91 | −0.600 | 2.04E−02 | −0.785 | 1.13E−15 | FALSE |
| Malat1 | −0.788 | 3.06E−83 | −0.680 | 1.62E−06 | −0.461 | 2.71E−11 | FALSE |
| Dnaja1 | −1.161 | 4.16E−78 | −0.342 | 9.15E−01 | −0.843 | 4.43E−12 | FALSE |
| Lmna | −1.945 | 1.16E−69 | −1.519 | 8.60E−04 | −0.532 | 2.38E−01 | FALSE |
| Hspa8 | −0.679 | 3.81E−68 | 0.219 | 9.15E−01 | −0.661 | 8.91E−16 | FALSE |
| Gm8730 | 0.782 | 1.16E−56 | 0.971 | 5.54E−06 | 0.633 | 6.80E−11 | FALSE |
| Rpl27-ps3 | 1.053 | 8.09E−50 | 0.805 | 2.26E−01 | 0.886 | 1.14E−15 | FALSE |
| Rgcc | −1.711 | 1.89E−45 | −0.813 | 6.43E−01 | −0.460 | 7.35E−01 | FALSE |
| Jund | −0.676 | 6.22E−42 | −0.962 | 8.04E−06 | −0.377 | 2.69E−03 | FALSE |
| Rpl35 | 0.509 | 2.46E−40 | 0.492 | 1.94E−02 | 0.489 | 2.42E−11 | FALSE |
| Gm10076 | 1.043 | 8.24E−36 | 0.361 | 1.00E+00 | 0.608 | 6.02E−04 | FALSE |
| Hspe1 | −0.936 | 2.25E−31 | −0.529 | 7.11E−01 | −0.858 | 2.83E−08 | FALSE |
| Rbm3 | 0.522 | 1.00E−30 | 0.689 | 2.20E−03 | 0.154 | 7.29E−01 | FALSE |
| Hspa1b | −1.577 | 8.12E−29 | −2.544 | 9.25E−10 | −1.026 | 1.09E−03 | FALSE |
| Rpl6I | 0.619 | 1.52E−27 | 0.448 | 7.04E−01 | 0.571 | 2.65E−08 | FALSE |
| Rpl9-ps6 | 0.729 | 4.55E−26 | 0.328 | 1.00E+00 | 0.736 | 1.87E−09 | FALSE |
| Plac8 | 1.004 | 1.74E−25 | 0.891 | 8.71E−02 | 0.027 | 1.00E+00 | FALSE |
| Rpl10-ps3 | 0.892 | 5.90E−25 | 0.219 | 1.00E+00 | 0.590 | 2.76E−03 | FALSE |
| Rplp0 | 0.288 | 2.35E−24 | 0.335 | 8.03E−02 | 0.162 | 5.42E−02 | FALSE |
| Gm2000 | 0.840 | 1.60E−23 | 0.555 | 8.76E−01 | 0.761 | 2.50E−06 | FALSE |
| AY036118 | 1.145 | 3.92E−23 | −0.264 | 1.00E+00 | 0.648 | 9.48E−03 | FALSE |
| Rps11 | 0.296 | 3.92E−23 | 0.236 | 5.32E−01 | 0.243 | 1.39E−04 | FALSE |
| Gm9843 | 0.527 | 2.80E−22 | 0.157 | 1.00E+00 | 0.679 | 2.47E−16 | FALSE |
| Crip1 | −0.543 | 9.93E−22 | −0.131 | 1.00E+00 | −0.778 | 1.45E−06 | FALSE |
| Atf3 | −1.251 | 9.16E−21 | −0.652 | 6.82E−01 | −0.067 | 1.00E+00 | FALSE |
| Fosb | −1.291 | 1.68E−20 | −0.525 | 4.01E−01 | −0.170 | 1.00E+00 | FALSE |
| Ccl5 | 1.130 | 1.94E−20 | 1.434 | 3.77E−04 | −0.184 | 7.29E−01 | FALSE |
| H2-D1 | −0.246 | 2.77E−18 | −0.296 | 1.15E−01 | −0.151 | 8.69E−02 | FALSE |
| Gzma | 1.725 | 5.05E−18 | 2.924 | 2.40E−10 | 1.884 | 2.33E−06 | FALSE |
| Capg | −0.679 | 5.65E−18 | −0.770 | 1.12E−01 | 0.058 | 1.00E+00 | FALSE |
| Hsph1 | −1.043 | 8.62E−18 | −0.588 | 9.15E−01 | −0.608 | 5.42E−02 | FALSE |
| AW112010 | 0.445 | 9.11E−18 | 0.011 | 1.00E+00 | −0.335 | 2.06E−02 | FALSE |
| Rps12-ps3 | 0.611 | 3.47E−17 | 0.168 | 1.00E+00 | 0.694 | 5.28E−08 | FALSE |
| Rpl23a-ps3 | 0.404 | 5.51E−17 | 0.279 | 9.15E−01 | 0.516 | 1.26E−08 | FALSE |
| Cd160 | 1.385 | 5.51E−17 | 1.394 | 4.54E−04 | 0.356 | 7.73E−01 | FALSE |
| Fth1 | −0.335 | 7.81E−17 | −0.305 | 6.82E−01 | −0.210 | 1.13E−01 | FALSE |
| Wdr89 | 0.370 | 1.58E−10 | 0.105 | 1.00E+00 | 0.742 | 2.47E−16 | FALSE |
| Rpl32 | 0.227 | 4.23E−16 | 0.259 | 2.34E−01 | 0.200 | 5.19E−04 | FALSE |
| Gm10073 | 0.680 | 5.48E−16 | 0.067 | 1.00E+00 | 0.567 | 5.13E−04 | FALSE |
| Gimap4 | 0.436 | 9.14E−16 | 0.030 | 1.00E+00 | 0.301 | 1.42E−02 | FALSE |
| Odc1 | −0.776 | 1.42E−15 | −0.233 | 1.00E+00 | −0.406 | 1.41E−01 | FALSE |
| Klf4 | −0.945 | 1.52E−15 | 0.000 | 1.00E+00 | −0.373 | 9.46E−01 | FALSE |
| Gm9493 | 0.455 | 2.68E−15 | 0.186 | 1.00E+00 | 0.476 | 8.63E−06 | FALSE |
| Epsti1 | 0.517 | 6.73E−15 | 0.125 | 1.00E+00 | 0.223 | 8.69E−01 | FALSE |
| Mat2a | −0.791 | 7.03E−15 | 0.545 | 9.50E−01 | −0.246 | 8.90E−01 | FALSE |
| Chchd2 | −0.377 | 7.45E−15 | −0.264 | 9.44E−01 | −0.256 | 1.72E−01 | FALSE |
| Eef2 | 0.257 | 6.16E−14 | 0.224 | 9.15E−01 | 0.279 | 5.69E−04 | FALSE |
| Hspd1 | −0.730 | 6.37E−14 | −0.022 | 1.00E+00 | −0.736 | 1.21E−04 | FALSE |
| Tnfsf11 | −1.017 | 6.37E−14 | −0.485 | 1.00E+00 | −0.517 | 2.38E−01 | FALSE |
| Rps27rt | 0.153 | 3.85E−02 | 0.017 | 1.00E+00 | 0.538 | 7.46E−14 | FALSE |
| Rpsa | 0.270 | 7.83E−14 | 0.161 | 9.96E−01 | 0.109 | 7.35E−01 | FALSE |
| Ubc | −0.410 | 8.17E−14 | −0.272 | 9.31E−01 | −0.333 | 1.87E−02 | FALSE |
| Limd2 | 0.519 | 1.01E−13 | 0.323 | 9.15E−01 | 0.021 | 1.00E+00 | FALSE |
| Ddx5 | −0.322 | 1.29E−13 | 0.096 | 1.00E+00 | −0.242 | 1.87E−02 | FALSE |
| Slc25a5 | 0.364 | 1.37E−13 | 0.436 | 4.18E−01 | 0.106 | 1.00E+00 | FALSE |
| Calm2 | −0.418 | 1.37E−13 | −0.269 | 9.43E−01 | −0.250 | 2.68E−01 | FALSE |
| Coro1a | 0.346 | 1.79E−13 | −0.211 | 9.15E−01 | 0.026 | 1.00E+00 | FALSE |
| Rpl7a-ps5 | 0.793 | 2.08E−13 | 0.495 | 9.82E−01 | 0.855 | 2.71E−05 | FALSE |
| Kdm6b | −0.839 | 2.14E−13 | −0.042 | 1.00E+00 | −0.228 | 9.94E−01 | FALSE |
| Cd7 | 1.097 | 2.81E−13 | 1.933 | 2.19E−05 | −0.262 | 1.00E+00 | FALSE |
| mt-Co1 | 0.235 | 1.31E−12 | 0.240 | 7.06E−01 | 0.134 | 5.30E−01 | FALSE |
| Gm11808 | 0.461 | 2.96E−12 | −0.153 | 1.00E+00 | 0.600 | 1.99E−08 | FALSE |
| Ptprcap | 0.468 | 3.65E−12 | 0.222 | 1.00E+00 | −0.099 | 1.00E+00 | FALSE |
| Rpl29 | 0.262 | 6.50E−12 | 0.377 | 2.07E−01 | 0.306 | 2.80E−04 | FALSE |
| Fam110a | −0.790 | 7.03E−12 | −0.231 | 1.00E+00 | −0.585 | 1.38E−01 | FALSE |
| Rpl39I | −0.660 | 1.20E−11 | 0.063 | 1.00E+00 | −0.011 | 1.00E+00 | FALSE |
| H2-K1 | −0.262 | 1.91E−11 | −0.387 | 1.01E−01 | −0.129 | 5.03E−01 | FALSE |
| Rpl15 | 0.305 | 2.06E−11 | 0.353 | 6.35E−01 | 0.300 | 5.49E−03 | FALSE |
| Rpl13-ps3 | 0.582 | 2.23E−11 | 0.125 | 1.00E+00 | 0.768 | 3.62E−07 | FALSE |
| Rps6 | 0.188 | 3.84E−11 | 0.218 | 6.23E−01 | 0.136 | 1.36E−01 | FALSE |
| Klrk1 | −0.696 | 9.06E−11 | −1.397 | 1.40E−01 | −0.182 | 1.00E+00 | FALSE |
| Lrrc58 | 0.664 | 1.14E−10 | 0.153 | 1.00E+00 | 0.342 | 5.86E−01 | FALSE |
| mt-Nd1 | −0.317 | 1.96E−10 | −0.384 | 4.52E−01 | −0.093 | 9.89E−01 | FALSE |
| Cdkn1a | −0.799 | 2.00E−10 | −0.160 | 1.00E+00 | −1.077 | 2.18E−05 | FALSE |
| Pycard | 0.552 | 3.15E−10 | 0.411 | 9.15E−01 | 0.196 | 9.47E−01 | FALSE |
| Rpl24 | 0.196 | 6.92E−10 | 0.424 | 5.06E−03 | 0.103 | 7.35E−01 | FALSE |
| Rps26-ps1 | 0.687 | 9.14E−10 | 0.339 | 1.00E+00 | 0.587 | 1.68E−02 | FALSE |
| Lgals1 | −0.300 | 1.31E−09 | −0.372 | 8.36E−01 | −0.200 | 8.31E−01 | FALSE |
| Rpl18 | 0.193 | 1.53E−09 | 0.239 | 5.56E−01 | 0.107 | 6.97E−01 | FALSE |
| Tuba1b | −0.597 | 1.83E−09 | −0.023 | 1.00E+00 | −0.159 | 1.00E+00 | FALSE |
| Anxa1 | −0.789 | 2.26E−09 | −0.508 | 1.00E+00 | −0.071 | 1.00E+00 | FALSE |
| Eif3f | 0.262 | 2.26E−09 | 0.628 | 1.66E−03 | 0.129 | 7.72E−01 | FALSE |
| Rps18-ps3 | 0.518 | 1.84E−06 | −0.035 | 1.00E+00 | 0.949 | 2.60E−09 | FALSE |
| Arhgdib | 0.265 | 2.99E−09 | 0.107 | 1.00E+00 | 0.070 | 1.00E+00 | FALSE |
| S100a11 | −0.309 | 3.72E−09 | −0.571 | 2.09E−02 | −0.140 | 9.38E−01 | FALSE |
| Gm10250 | 0.669 | 4.08E−09 | 0.479 | 9.50E−01 | 0.594 | 2.22E−02 | FALSE |
| Actg1 | 0.252 | 5.73E−09 | −0.085 | 1.00E+00 | −0.255 | 4.14E−02 | FALSE |
| Rgs10 | 0.576 | 7.56E−09 | 0.509 | 8.47E−01 | 0.223 | 1.00E+00 | FALSE |
| Cox7a2l | 0.344 | 9.72E−09 | 0.562 | 2.23E−01 | 0.225 | 5.06E−01 | FALSE |
| Ifrd1 | −0.475 | 1.07E−08 | −0.537 | 9.15E−01 | −0.207 | 9.46E−01 | FALSE |
| B2m | −0.150 | 1.93E−08 | −0.197 | 5.39E−01 | −0.153 | 8.64E−03 | FALSE |
| Gm10020 | 0.682 | 2.04E−08 | 0.130 | 1.00E+00 | 0.394 | 4.80E−01 | FALSE |
| Hspa5 | −0.331 | 2.08E−08 | −0.159 | 1.00E+00 | −0.539 | 9.48E−07 | FALSE |
| Cd44 | −0.531 | 2.73E−08 | −0.394 | 1.00E+00 | 0.199 | 9.77E−01 | FALSE |
| Id2 | −0.415 | 4.68E−08 | −0.270 | 9.15E−01 | −0.485 | 1.10E−02 | FALSE |
| Shisa5 | 0.242 | 5.38E−08 | 0.220 | 9.15E−01 | 0.160 | 6.70E−01 | FALSE |
| Srsf2 | −0.454 | 5.55E−08 | −0.145 | 1.00E+00 | −0.231 | 7.35E−01 | FALSE |
| Itgae | 0.822 | 6.06E−08 | 1.438 | 2.78E−03 | 0.097 | 1.00E+00 | FALSE |
| H2-Q7 | −0.287 | 6.64E−08 | −0.330 | 9.15E−01 | −0.204 | 2.09E−01 | FALSE |
| Nmrk1 | −0.649 | 6.64E−08 | −0.216 | 1.00E+00 | −0.349 | 7.39E−01 | FALSE |
| Gm10036 | 0.543 | 7.07E−08 | 0.284 | 1.00E+00 | 0.599 | 3.19E−03 | FALSE |
| Sh3bgrl3 | −0.214 | 7.55E−08 | −0.185 | 9.15E−01 | −0.135 | 8.78E−01 | FALSE |
| Txn1 | −0.358 | 7.94E−08 | −0.019 | 1.00E+00 | −0.413 | 1.07E−02 | FALSE |
| Ly6e | −0.296 | 1.04E−07 | −0.168 | 1.00E+00 | −0.115 | 1.00E+00 | FALSE |
| Pim1 | −0.384 | 1.17E−07 | 1.112 | 7.90E−02 | −0.147 | 1.00E+00 | FALSE |
| Rpl22l1 | 0.264 | 1.17E−07 | 0.667 | 2.78E−03 | 0.241 | 1.31E−01 | FALSE |
| Prr7 | −0.462 | 1.18E−07 | −0.038 | 1.00E+00 | −0.359 | 6.64E−01 | FALSE |
| Rpl13a | 0.112 | 8.57E−04 | 0.204 | 5.26E−01 | 0.253 | 1.19E−07 | FALSE |
| Ifitm1 | −1.211 | 2.18E−07 | −0.065 | 1.00E+00 | −0.042 | 1.00E+00 | FALSE |
| Sertad1 | −0.661 | 2.31E−07 | −0.315 | 1.00E+00 | −0.515 | 1.82E−01 | FALSE |
| Ybx1 | −0.266 | 2.52E−07 | −0.190 | 1.00E+00 | −0.142 | 8.82E−01 | FALSE |
| Cldnd1 | −0.520 | 2.52E−07 | −1.374 | 2.60E−04 | −0.132 | 1.00E+00 | FALSE |
| Tmem234 | 0.427 | 3.46E−07 | 0.378 | 9.96E−01 | 0.107 | 1.00E+00 | FALSE |
| Bola2 | −0.480 | 3.67E−07 | −0.411 | 9.33E−01 | −0.111 | 1.00E+00 | FALSE |
| Serpinb6b | 0.564 | 3.86E−07 | 0.097 | 1.00E+00 | 0.312 | 8.36E−01 | FALSE |
| Gm6133 | 0.402 | 3.94E−07 | 0.500 | 8.71E−01 | 0.658 | 3.94E−07 | FALSE |
| Rps3a1 | 0.171 | 4.56E−07 | 0.190 | 9.15E−01 | 0.229 | 1.32E−03 | FALSE |
| Ccr9 | 0.870 | 4.80E−06 | 2.222 | 5.15E−07 | 0.716 | 1.43E−01 | FALSE |
| Cacybp | −0.535 | 6.43E−07 | −0.424 | 9.96E−01 | −0.502 | 6.32E−02 | FALSE |
| Tspan31 | −0.578 | 7.07E−07 | 0.251 | 1.00E+00 | −0.153 | 1.00E+00 | FALSE |
| Gmfg | 0.309 | 7.13E−07 | 0.002 | 1.00E+00 | 0.102 | 1.00E+00 | FALSE |
| Gm12840 | 0.587 | 7.30E−07 | 0.093 | 1.00E+00 | −0.072 | 1.00E+00 | FALSE |
| Acp5 | 0.557 | 8.50E−07 | 0.657 | 7.11E−01 | 0.205 | 1.00E+00 | FALSE |
| Cd69 | −0.535 | 8.50E−07 | −0.128 | 1.00E+00 | 0.325 | 7.35E−01 | FALSE |
| Rps28 | 0.023 | 9.39E−01 | 0.234 | 8.27E−01 | 0.346 | 8.78E−07 | FALSE |
| Cd82 | −0.350 | 1.39E−06 | −0.388 | 9.15E−01 | 0.045 | 1.00E+00 | FALSE |
| Rps19 | 0.000 | 1.00E+00 | 0.135 | 1.00E+00 | 0.309 | 1.69E−06 | FALSE |
| Gm10709 | 0.581 | 1.74E−06 | 0.044 | 1.00E+00 | 0.313 | 6.24E−01 | FALSE |
| Cox4i1 | 0.210 | 1.86E−06 | 0.261 | 8.76E−01 | 0.053 | 1.00E+00 | FALSE |
| Gzmb | 0.744 | 1.86E−06 | 0.314 | 1.00E+00 | −0.177 | 1.00E+00 | FALSE |
| Il22 | −0.801 | 2.21E−06 | −0.122 | 1.00E+00 | −0.588 | 3.81E−02 | FALSE |
| Calm1 | −0.233 | 2.38E−06 | −0.152 | 1.00E+00 | −0.075 | 1.00E+00 | FALSE |
| Zc3h12a | −0.459 | 2.45E−06 | −0.204 | 1.00E+00 | 0.018 | 1.00E+00 | FALSE |
| Ier5l | −0.608 | 2.59E−06 | 0.058 | 1.00E+00 | −0.481 | 4.56E−01 | FALSE |
| Fus | −0.382 | 3.48E−06 | −0.622 | 4.98E−01 | −0.343 | 1.31E−01 | FALSE |
| Klhl6 | 0.545 | 3.88E−06 | 0.258 | 1.00E+00 | 0.321 | 9.46E−01 | FALSE |
| Atp2b1 | −0.507 | 4.14E−06 | −0.202 | 1.00E+00 | −0.344 | 6.89E−01 | FALSE |
| Tbc1d10c | 0.448 | 4.33E−06 | 0.319 | 9.28E−01 | 0.419 | 1.97E−01 | FALSE |
| H2-Q6 | −0.388 | 4.58E−06 | −0.416 | 9.15E−01 | −0.205 | 8.35E−01 | FALSE |
| Ctla2a | −0.437 | 5.15E−06 | −0.890 | 2.05E−01 | −0.240 | 8.18E−01 | FALSE |
| Cnn2 | 0.317 | 5.69E−06 | 0.169 | 1.00E+00 | 0.139 | 1.00E+00 | FALSE |
| Selplg | 0.263 | 6.24E−06 | 0.320 | 9.15E−01 | 0.298 | 2.54E−01 | FALSE |
| Apobec3 | 0.443 | 7.33E−06 | 0.324 | 1.00E+00 | 0.170 | 1.00E+00 | FALSE |
| Crip2 | −0.681 | 8.46E−06 | 0.156 | 1.00E+00 | −0.339 | 6.80E−01 | FALSE |
| Bcl2a1a | 0.582 | 8.84E−06 | 0.326 | 9.15E−01 | −0.028 | 1.00E+00 | FALSE |
| Hist1h1c | 0.371 | 9.01E−02 | −0.174 | 1.00E+00 | 1.117 | 1.07E−05 | FALSE |
| Ifitm2 | −0.485 | 1.18E−05 | −0.775 | 6.49E−01 | −0.027 | 1.00E+00 | FALSE |
| Eif5 | −0.383 | 1.28E−05 | −0.338 | 1.00E+00 | −0.187 | 8.82E−01 | FALSE |
| Ppia | −0.172 | 1.38E−05 | −0.314 | 2.26E−01 | −0.053 | 1.00E+00 | FALSE |
| Satb1 | 0.475 | 1.39E−05 | −0.205 | 1.00E+00 | 0.324 | 7.61E−01 | FALSE |
| Rps5 | 0.028 | 8.66E−01 | 0.096 | 1.00E+00 | 0.258 | 1.39E−05 | FALSE |
| Arhgap9 | 0.469 | 1.60E−05 | 0.213 | 1.00E+00 | 0.467 | 9.90E−02 | FALSE |
| Eif2s3y | 0.559 | 1.63E−05 | 0.762 | 6.37E−01 | 0.492 | 1.87E−01 | FALSE |
| Sub1 | −0.158 | 6.18E−03 | −0.302 | 8.30E−01 | −0.380 | 1.71E−05 | FALSE |
| Tagln2 | −0.242 | 9.64E−04 | −0.971 | 1.72E−05 | 0.083 | 1.00E+00 | FALSE |
| Jun | −0.681 | 1.22E−03 | −1.899 | 2.03E−05 | 0.149 | 1.00E+00 | FALSE |
| Rpl6 | 0.138 | 2.04E−05 | 0.169 | 9.15E−01 | 0.244 | 3.06E−04 | FALSE |
| Ptges3 | −0.451 | 2.22E−05 | −0.287 | 1.00E+00 | −0.122 | 1.00E+00 | FALSE |
| Ccdc184 | −0.532 | 2.42E−05 | −0.178 | 9.15E−01 | −0.033 | 1.00E+00 | FALSE |
| Eif4a2 | −0.402 | 2.49E−05 | −0.396 | 9.15E−01 | 0.040 | 1.00E+00 | FALSE |
| Rgs2 | −0.520 | 2.53E−05 | −0.607 | 9.15E−01 | −0.616 | 3.81E−02 | FALSE |
| Actn2 | 0.565 | 2.95E−05 | 0.175 | 1.00E+00 | 0.383 | 7.61E−01 | FALSE |
| Jmjd1c | −0.520 | 3.37E−05 | −0.004 | 1.00E+00 | −0.343 | 7.35E−01 | FALSE |
| Ass1 | 0.465 | 3.41E−05 | 1.193 | 8.71E−02 | −0.025 | 1.00E+00 | FALSE |
| Rpl21 | 0.004 | 1.00E+00 | −0.013 | 1.00E+00 | 0.280 | 3.44E−05 | FALSE |
| Tubb4b | −0.507 | 3.63E−05 | 0.023 | 1.00E+00 | −0.338 | 7.29E−01 | FALSE |
| Gramd1a | 0.446 | 3.76E−05 | 0.451 | 9.15E−01 | 0.157 | 1.00E+00 | FALSE |
| Rps10 | 0.160 | 4.16E−05 | 0.207 | 9.15E−01 | 0.232 | 9.89E−03 | FALSE |
| mt-Cytb | −0.203 | 4.75E−05 | −0.410 | 2.07E−01 | −0.177 | 6.78E−01 | FALSE |
| Hmgb2 | 0.286 | 4.75E−05 | −0.076 | 1.00E+00 | 0.025 | 1.00E+00 | FALSE |
| Rpl36-ps3 | 0.337 | 7.29E−03 | 0.158 | 1.00E+00 | 0.696 | 4.88E−05 | FALSE |
| Sp100 | 0.392 | 5.36E−05 | 0.196 | 1.00E+00 | 0.355 | 2.26E−01 | FALSE |
| Vgll4 | −0.413 | 5.44E−05 | 0.179 | 1.00E+00 | −0.248 | 8.30E−01 | FALSE |
| Rinl | 0.443 | 6.06E−05 | −0.003 | 1.00E+00 | 0.315 | 6.24E−01 | FALSE |
| Gem | −0.687 | 6.58E−05 | −0.047 | 1.00E+00 | −0.785 | 5.35E−02 | FALSE |
| Stk17b | 0.285 | 6.58E−05 | 0.217 | 1.00E+00 | −0.007 | 1.00E+00 | FALSE |
| Unc5cl | 0.525 | 6.58E−05 | 0.511 | 9.15E−01 | −0.257 | 8.90E−01 | FALSE |
| Lck | 0.228 | 6.70E−05 | 0.086 | 1.00E+00 | 0.178 | 5.86E−01 | FALSE |
| Gltscr2 | 0.253 | 6.94E−05 | 0.453 | 7.11E−01 | 0.092 | 1.00E+00 | FALSE |
| Ict1 | 0.464 | 6.94E−05 | 0.038 | 1.00E+00 | 0.288 | 7.39E−01 | FALSE |
| Rpl10a | 0.178 | 7.01E−05 | 0.338 | 4.62E−01 | 0.199 | 8.74E−02 | FALSE |
| Gpi1 | 0.328 | 7.47E−05 | 0.231 | 1.00E+00 | −0.080 | 1.00E+00 | FALSE |
| Ltb | 0.339 | 7.56E−05 | 0.547 | 7.11E−01 | 0.241 | 7.35E−01 | FALSE |
| Tmsb4x | −0.094 | 5.14E−03 | −0.213 | 2.65E−01 | −0.251 | 8.21E−05 | FALSE |
| Calr | −0.310 | 8.23E−05 | −0.523 | 7.11E−01 | −0.162 | 9.46E−01 | FALSE |
| Cycs | −0.330 | 8.59E−05 | −0.521 | 7.60E−01 | −0.294 | 5.16E−01 | FALSE |
| Cpm | −0.558 | 1.03E−04 | 0.220 | 9.96E−01 | −0.358 | 5.63E−01 | FALSE |
| Madd | 0.465 | 1.10E−04 | −0.029 | 1.00E+00 | 0.173 | 1.00E+00 | FALSE |
| Anxa2 | −0.351 | 1.11E−04 | −0.462 | 9.15E−01 | −0.193 | 9.77E−01 | FALSE |
| Chn2 | 0.571 | 1.11E−04 | 0.645 | 8.59E−01 | −0.004 | 1.00E+00 | FALSE |
| Ube2s | −0.304 | 1.17E−04 | −0.578 | 6.42E−01 | −0.208 | 7.35E−01 | FALSE |
| Atp5g1 | −0.344 | 1.24E−04 | −0.227 | 1.00E+00 | −0.045 | 1.00E+00 | FALSE |
| Tuba4a | −0.504 | 1.25E−04 | −0.415 | 1.00E+00 | −0.435 | 4.02E−01 | FALSE |
| Gm9844 | 0.390 | 1.29E−04 | −0.103 | 1.00E+00 | 0.307 | 7.04E−01 | FALSE |
| Uba52 | 0.273 | 1.30E−04 | −0.143 | 1.00E+00 | 0.420 | 5.40E−04 | FALSE |
| Il21r | −0.350 | 1.39E−04 | 0.082 | 1.00E+00 | −0.455 | 1.09E−01 | FALSE |
| Rpl13 | 0.043 | 5.16E−01 | 0.116 | 1.00E+00 | 0.207 | 1.39E−04 | FALSE |
| Rps24 | −0.020 | 9.33E−01 | 0.172 | 9.15E−01 | 0.242 | 1.48E−04 | FALSE |
| mt-Nd2 | −0.311 | 1.70E−04 | −0.428 | 9.15E−01 | −0.059 | 1.00E+00 | FALSE |
| Eif4a1 | −0.219 | 1.85E−04 | 0.476 | 5.04E−01 | −0.152 | 8.26E−01 | FALSE |
| Ifi27 | −0.389 | 1.90E−04 | −0.811 | 1.03E−01 | −0.310 | 6.69E−01 | FALSE |
| Fau | 0.018 | 9.31E−01 | 0.069 | 1.00E+00 | 0.200 | 1.92E−04 | FALSE |
| Bcl2l1 | −0.508 | 1.97E−04 | 0.300 | 1.00E+00 | −0.394 | 6.06E−01 | FALSE |
| Furin | −0.428 | 2.07E−04 | −0.818 | 5.16E−01 | −0.120 | 1.00E+00 | FALSE |
| Serf2 | −0.177 | 2.12E−04 | −0.289 | 6.92E−01 | −0.081 | 1.00E+00 | FALSE |
| Gm10263 | 0.514 | 2.21E−04 | 0.320 | 1.00E+00 | 0.319 | 7.35E−01 | FALSE |
| Rasal3 | 0.453 | 2.31E−04 | 0.084 | 1.00E+00 | 0.217 | 9.46E−01 | FALSE |
| Frmd4b | −0.570 | 2.34E−04 | 0.034 | 1.00E+00 | −0.402 | 3.83E−01 | FALSE |
| Tnfrsf4 | −0.387 | 2.59E−04 | −0.559 | 9.15E−01 | −0.768 | 7.68E−04 | FALSE |
| H1f0 | 0.574 | 2.68E−04 | −0.545 | 9.15E−01 | 0.441 | 7.35E−01 | FALSE |
| Bin2 | 0.322 | 2.72E−04 | 0.800 | 2.52E−01 | 0.338 | 4.67E−01 | FALSE |
| Lztfl1 | 0.556 | 2.81E−04 | 0.477 | 9.59E−01 | 0.462 | 5.49E−01 | FALSE |
| Rpl18a | 0.033 | 6.68E−01 | 0.190 | 7.11E−01 | 0.194 | 2.95E−04 | FALSE |
| Klrc2 | −0.516 | 3.06E−04 | −0.324 | 1.00E+00 | −0.068 | 1.00E+00 | FALSE |
| Rps23 | −0.023 | 9.01E−01 | 0.175 | 8.17E−01 | 0.192 | 3.35E−04 | FALSE |
| Irf7 | −0.557 | 3.50E−04 | −0.108 | 1.00E+00 | −0.188 | 1.00E+00 | FALSE |
| Ypel3 | 0.379 | 3.68E−04 | −0.191 | 1.00E+00 | 0.089 | 1.00E+00 | FALSE |
| Sap30 | −0.491 | 3.79E−04 | −0.177 | 1.00E+00 | 0.023 | 1.00E+00 | FALSE |
| Csk | 0.418 | 3.92E−04 | 0.021 | 1.00E+00 | 0.031 | 1.00E+00 | FALSE |
| Uhrf2 | −0.455 | 4.30E−04 | −0.840 | 5.49E−01 | −0.097 | 1.00E+00 | FALSE |
| Romo1 | −0.379 | 4.47E−04 | −0.138 | 1.00E+00 | −0.258 | 7.62E−01 | FALSE |
| Agr2 | −0.159 | 4.57E−04 | 0.061 | 1.00E+00 | 0.000 | 1.00E+00 | FALSE |
| Il17a | −0.068 | 5.25E−01 | −1.985 | 4.62E−04 | −0.031 | 1.00E+00 | FALSE |
| Tnf | −0.450 | 4.66E−04 | 0.257 | 1.00E+00 | −0.009 | 1.00E+00 | FALSE |
| Gimap5 | 0.365 | 4.76E−04 | 0.260 | 1.00E+00 | 0.304 | 4.85E−01 | FALSE |
| Maff | −0.514 | 5.01E−04 | 0.000 | 1.00E+00 | 0.057 | 1.00E+00 | FALSE |
| Ptma | −0.190 | 5.61E−04 | −0.201 | 1.00E+00 | −0.178 | 3.32E−01 | FALSE |
| Ppid | −0.465 | 5.99E−04 | −0.153 | 1.00E+00 | −0.236 | 9.46E−01 | FALSE |
| Rpl17 | 0.019 | 9.31E−01 | 0.113 | 1.00E+00 | 0.194 | 6.89E−04 | FALSE |
| Rpl31 | 0.157 | 8.80E−03 | 0.091 | 1.00E+00 | 0.336 | 6.95E−04 | FALSE |
| Ccl1 | −0.840 | 7.05E−04 | −0.183 | 1.00E+00 | −0.043 | 1.00E+00 | FALSE |
| Nr3c1 | −0.464 | 7.10E−04 | −0.208 | 1.00E+00 | −0.183 | 1.00E+00 | FALSE |
| Rasgrp4 | −0.291 | 3.75E−02 | −1.110 | 7.22E−04 | 0.070 | 9.80E−01 | FALSE |
| Rnf166 | 0.410 | 7.68E−04 | 0.114 | 1.00E+00 | 0.282 | 7.35E−01 | FALSE |
| Coq10b | −0.414 | 7.70E−04 | −0.038 | 1.00E+00 | −0.243 | 7.99E−01 | FALSE |
| Chka | −0.447 | 8.50E−04 | 0.061 | 1.00E+00 | −0.138 | 1.00E+00 | FALSE |
| Cotl1 | 0.231 | 8.55E−04 | 0.017 | 1.00E+00 | −0.043 | 1.00E+00 | FALSE |
| Cd47 | 0.207 | 9.38E−04 | 0.187 | 1.00E+00 | 0.210 | 7.35E−01 | FALSE |
| Ralgds | −0.442 | 9.42E−04 | 0.199 | 1.00E+00 | −0.194 | 9.77E−01 | FALSE |
| Arpc5 | 0.235 | 9.42E−04 | 0.010 | 1.00E+00 | −0.173 | 8.55E−01 | FALSE |
| Samsn1 | −0.352 | 9.42E−04 | −0.418 | 1.00E+00 | −0.180 | 1.00E+00 | FALSE |
| Dgka | 0.449 | 9.42E−04 | −0.189 | 1.00E+00 | 0.185 | 1.00E+00 | FALSE |
| Cd40lg | −0.455 | 9.53E−04 | −0.173 | 1.00E+00 | −0.187 | 1.00E+00 | FALSE |
| BC021614 | 0.460 | 9.64E−04 | 0.777 | 7.25E−01 | 0.087 | 1.00E+00 | FALSE |
| Tsc22d3 | 0.336 | 9.41E−03 | 0.415 | 1.00E+00 | 0.729 | 9.86E−04 | FALSE |
| Sfn | −0.582 | 9.92E−04 | −0.544 | 7.82E−01 | −0.498 | 4.50E−01 | FALSE |
| Hnrnpa3 | 0.229 | 1.00E−03 | −0.019 | 1.00E+00 | 0.038 | 1.00E+00 | FALSE |
| Gm42726 | 0.400 | 1.15E−03 | 0.275 | 9.15E−01 | 0.553 | 4.64E−02 | FALSE |
| Ccnd2 | −0.336 | 1.15E−03 | −0.198 | 1.00E+00 | −0.473 | 8.21E−02 | FALSE |
| Rps29 | 0.070 | 2.05E−01 | 0.193 | 9.15E−01 | 0.241 | 1.22E−03 | FALSE |
| Rpl14 | −0.002 | 1.00E+00 | 0.048 | 1.00E+00 | 0.222 | 1.23E−03 | FALSE |
| Gm5424 | 0.407 | 1.24E−03 | 0.493 | 6.82E−01 | 0.013 | 1.00E+00 | FALSE |
| Eif1 | −0.128 | 1.29E−03 | −0.146 | 1.00E+00 | −0.063 | 9.81E−01 | FALSE |
| Bag3 | −0.406 | 1.29E−03 | −0.115 | 1.00E+00 | −0.500 | 1.69E−01 | FALSE |
| Eif5a | −0.213 | 1.36E−03 | −0.025 | 1.00E+00 | −0.186 | 7.35E−01 | FALSE |
| Slc5a3 | −0.354 | 1.41E−03 | 0.000 | 1.00E+00 | −0.174 | 1.00E+00 | FALSE |
| Traf4 | −0.468 | 1.42E−03 | 0.135 | 1.00E+00 | −0.030 | 1.00E+00 | FALSE |
| Traf3ip3 | 0.439 | 1.42E−03 | 0.463 | 9.74E−01 | 0.429 | 3.54E−01 | FALSE |
| Mrps6 | −0.503 | 1.42E−03 | 0.144 | 1.00E+00 | −0.431 | 6.93E−01 | FALSE |
| Nfkbia | −0.266 | 1.43E−03 | −0.226 | 1.00E+00 | 0.030 | 1.00E+00 | FALSE |
| Rps27 | −0.114 | 1.53E−03 | 0.050 | 1.00E+00 | 0.173 | 1.69E−02 | FALSE |
| Cd38 | 0.495 | 1.66E−03 | 0.176 | 1.00E+00 | −0.090 | 1.00E+00 | FALSE |
| Gimap7 | 0.261 | 1.67E−03 | 0.433 | 7.11E−01 | −0.016 | 1.00E+00 | FALSE |
| Lcp1 | 0.222 | 1.68E−03 | 0.064 | 1.00E+00 | 0.139 | 9.46E−01 | FALSE |
| Gpr171 | 0.316 | 1.71E−03 | 0.317 | 1.00E+00 | 0.039 | 1.00E+00 | FALSE |
| Gpsm3 | 0.278 | 1.72E−03 | −0.308 | 9.34E−01 | 0.162 | 9.46E−01 | FALSE |
| Tuba1a | −0.333 | 1.72E−03 | −0.654 | 5.39E−01 | −0.328 | 6.03E−01 | FALSE |
| Gm8186 | 0.417 | 1.72E−03 | 0.148 | 1.00E+00 | 0.284 | 7.56E−01 | FALSE |
| Ctbp1 | 0.350 | 1.72E−03 | 0.181 | 1.00E+00 | 0.040 | 1.00E+00 | FALSE |
| Tcf7 | 0.426 | 1.76E−03 | −0.109 | 1.00E+00 | 0.256 | 9.37E−01 | FALSE |
| Ndufs5 | −0.348 | 1.76E−03 | −0.573 | 7.11E−01 | −0.148 | 1.00E+00 | FALSE |
| Fasl | −0.351 | 1.76E−03 | 0.453 | 1.00E+00 | −0.156 | 1.00E+00 | FALSE |
| Ier3ip1 | −0.331 | 1.76E−03 | −0.268 | 1.00E+00 | 0.084 | 1.00E+00 | FALSE |
| Itpkb | −0.409 | 1.80E−03 | 0.716 | 8.04E−01 | −0.035 | 1.00E+00 | FALSE |
| Rpl4 | 0.115 | 1.80E−03 | 0.137 | 1.00E+00 | 0.137 | 2.87E−01 | FALSE |
| Ccnl1 | −0.372 | 2.03E−03 | 0.028 | 1.00E+00 | −0.276 | 7.39E−01 | FALSE |
| Cd48 | 0.286 | 2.03E−03 | 0.114 | 1.00E+00 | −0.043 | 1.00E+00 | FALSE |
| Hnrnpab | −0.358 | 2.03E−03 | 0.257 | 1.00E+00 | −0.292 | 7.35E−01 | FALSE |
| Insig1 | −0.488 | 2.03E−03 | 0.553 | 9.68E−01 | −0.456 | 3.80E−01 | FALSE |
| Tceb2 | −0.198 | 2.03E−03 | −0.050 | 1.00E+00 | −0.130 | 9.46E−01 | FALSE |
| Gm5093 | 0.340 | 2.15E−02 | −0.071 | 1.00E+00 | 0.630 | 2.06E−03 | FALSE |
| Tpm4 | 0.336 | 2.10E−03 | −0.086 | 1.00E+00 | 0.312 | 7.35E−01 | FALSE |
| Ahsa1 | −0.379 | 2.15E−03 | 0.328 | 1.00E+00 | −0.250 | 8.11E−01 | FALSE |
| Haghl | 0.414 | 2.15E−03 | −0.073 | 1.00E+00 | 0.013 | 1.00E+00 | FALSE |
| Rpl8 | 0.118 | 2.22E−03 | 0.207 | 8.72E−01 | 0.226 | 2.89E−03 | FALSE |
| Tob1 | −0.420 | 2.23E−03 | 0.073 | 1.00E+00 | −0.367 | 7.29E−01 | FALSE |
| Nop10 | −0.316 | 2.23E−03 | −0.495 | 7.75E−01 | −0.339 | 3.59E−01 | FALSE |
| Nsa2 | 0.337 | 2.28E−03 | 0.175 | 1.00E+00 | 0.020 | 1.00E+00 | FALSE |
| Nasp | −0.429 | 2.31E−03 | −0.047 | 1.00E+00 | 0.232 | 9.46E−01 | FALSE |
| Il4ra | −0.403 | 2.35E−03 | 0.012 | 1.00E+00 | −0.137 | 1.00E+00 | FALSE |
| Rac2 | 0.145 | 2.40E−03 | −0.118 | 1.00E+00 | 0.090 | 9.46E−01 | FALSE |
| Gm11273 | 0.410 | 2.41E−03 | 0.413 | 1.00E+00 | 0.163 | 1.00E+00 | FALSE |
| Cd4 | −0.070 | 9.13E−01 | −0.953 | 2.41E−03 | 0.120 | 1.00E+00 | FALSE |
| Arf4 | −0.256 | 2.52E−03 | −0.236 | 1.00E+00 | −0.350 | 1.59E−01 | FALSE |
| Supt4a | 0.231 | 2.64E−03 | 0.021 | 1.00E+00 | 0.131 | 9.97E−01 | FALSE |
| Tmsb10 | −0.111 | 2.66E−03 | −0.469 | 8.84E−03 | 0.103 | 8.47E−01 | FALSE |
| Nab1 | 0.378 | 2.66E−03 | 0.461 | 1.00E+00 | 0.123 | 1.00E+00 | FALSE |
| Rps18 | 0.107 | 3.65E−03 | 0.136 | 9.37E−01 | 0.206 | 2.69E−03 | FALSE |
| Gm10260 | 0.230 | 2.14E−01 | 0.200 | 1.00E+00 | 0.643 | 2.69E−03 | FALSE |
| Ly6a | −0.125 | 5.25E−01 | −0.565 | 2.23E−01 | −0.647 | 2.74E−03 | FALSE |
| Ppp1cc | 0.008 | 1.00E+00 | 0.247 | 1.00E+00 | −0.500 | 2.74E−03 | FALSE |
| Gm26917 | 0.351 | 3.02E−03 | 0.441 | 9.15E−01 | 0.539 | 9.89E−03 | FALSE |
| Gm10269 | 0.401 | 3.16E−03 | 0.159 | 1.00E+00 | 0.267 | 7.97E−01 | FALSE |
| Sema4d | 0.376 | 3.19E−03 | 0.077 | 1.00E+00 | −0.148 | 1.00E+00 | FALSE |
| Rpl34 | 0.044 | 5.40E−01 | 0.073 | 1.00E+00 | 0.193 | 3.32E−03 | FALSE |
| Ap2s1 | 0.315 | 3.87E−03 | −0.121 | 1.00E+00 | 0.012 | 1.00E+00 | FALSE |
| Lag3 | −0.110 | 9.31E−01 | −0.703 | 8.71E−01 | −1.023 | 3.92E−03 | FALSE |
| Hbegf | −0.210 | 3.96E−03 | 0.000 | 1.00E+00 | −0.054 | 9.77E−01 | FALSE |
| Tsix | 0.391 | 3.96E−03 | 0.221 | 9.15E−01 | 0.099 | 1.00E+00 | FALSE |
| Btf3 | 0.145 | 4.03E−03 | 0.201 | 9.75E−01 | 0.079 | 1.00E+00 | FALSE |
| Gm42418 | 0.397 | 4.06E−03 | −0.702 | 7.35E−01 | 0.160 | 1.00E+00 | FALSE |
| 3830403N18Rik | 0.436 | 4.08E−03 | 0.617 | 6.82E−01 | 0.045 | 9.46E−01 | FALSE |
| Hk2 | −0.498 | 4.17E−03 | −0.358 | 9.17E−01 | 0.037 | 1.00E+00 | FALSE |
| Csf1 | −0.559 | 4.41E−03 | −0.831 | 2.44E−01 | −0.107 | 1.00E+00 | FALSE |
| Cxcr3 | 0.369 | 4.42E−03 | 0.544 | 8.76E−01 | 0.085 | 1.00E+00 | FALSE |
| Mt1 | 0.658 | 4.42E−03 | 0.321 | 9.15E−01 | −0.354 | 9.46E−01 | FALSE |
| Nkg7 | 0.163 | 4.42E−03 | 0.126 | 1.00E+00 | −0.166 | 4.19E−01 | FALSE |
| Nudc | −0.360 | 4.43E−03 | 0.092 | 1.00E+00 | −0.028 | 1.00E+00 | FALSE |
| Vasp | 0.214 | 4.50E−03 | 0.150 | 1.00E+00 | 0.011 | 1.00E+00 | FALSE |
| Cd9 | −0.494 | 4.78E−03 | −0.244 | 1.00E+00 | 0.244 | 9.77E−01 | FALSE |
| Gzmk | 0.579 | 7.04E−03 | 1.524 | 5.05E−03 | 0.280 | 3.28E−01 | FALSE |
| Actr3 | 0.192 | 5.14E−03 | 0.060 | 1.00E+00 | −0.017 | 1.00E+00 | FALSE |
| Lef1 | 0.365 | 2.75E−02 | −0.360 | 1.00E+00 | 0.708 | 5.26E−03 | FALSE |
| Gm20186 | 0.449 | 5.31E−03 | −0.193 | 1.00E+00 | 0.177 | 1.00E+00 | FALSE |
| 1810041H14Rik | 0.428 | 5.38E−03 | 1.449 | 1.80E−02 | −0.083 | 1.00E+00 | FALSE |
| Pdlim2 | 0.408 | 5.65E−03 | 1.038 | 1.88E−01 | 0.589 | 4.34E−02 | FALSE |
| 1600014C10Rik | 0.431 | 5.93E−03 | 0.217 | 1.00E+00 | −0.037 | 1.00E+00 | FALSE |
| Cyp51 | −0.427 | 5.93E−03 | 0.596 | 9.15E−01 | −0.344 | 7.35E−01 | FALSE |
| Prdx1 | −0.237 | 5.93E−03 | −0.475 | 7.11E−01 | −0.336 | 1.78E−01 | FALSE |
| Sigmar1 | −0.319 | 5.95E−03 | −0.198 | 1.00E+00 | −0.106 | 1.00E+00 | FALSE |
| 1810058124Rik | 0.323 | 6.04E−03 | −0.052 | 1.00E+00 | 0.148 | 1.00E+00 | FALSE |
| Rps7 | 0.104 | 6.04E−03 | 0.186 | 9.15E−01 | 0.176 | 2.29E−02 | FALSE |
| Eno1 | 0.233 | 6.04E−03 | −0.361 | 9.15E−01 | 0.324 | 2.78E−01 | FALSE |
| Ndufa6 | 0.227 | 6.06E−03 | 0.140 | 1.00E+00 | 0.180 | 8.39E−01 | FALSE |
| Gm10146 | 0.151 | 6.18E−03 | −0.119 | 1.00E+00 | 0.076 | 1.00E+00 | FALSE |
| Dusp14 | −0.426 | 6.18E−03 | 0.324 | 9.15E−01 | −0.222 | 9.77E−01 | FALSE |
| Il18r1 | 0.033 | 1.00E+00 | −1.158 | 6.35E−03 | 0.235 | 9.46E−01 | FALSE |
| Cuedc2 | 0.428 | 6.51E−03 | −0.185 | 1.00E+00 | 0.051 | 1.00E+00 | FALSE |
| Gyg | 0.417 | 6.55E−03 | 0.060 | 1.00E+00 | 0.031 | 1.00E+00 | FALSE |
| Ptafr | −0.416 | 6.56E−03 | −0.186 | 9.15E−01 | 0.025 | 1.00E+00 | FALSE |
| Rpl13a-ps1 | 0.322 | 4.64E−02 | −0.227 | 1.00E+00 | 0.637 | 6.57E−03 | FALSE |
| Klrb1f | 0.353 | 6.58E−03 | 0.484 | 9.15E−01 | 0.020 | 1.00E+00 | FALSE |
| Psme2b | 0.400 | 6.68E−03 | −0.054 | 1.00E+00 | 0.240 | 9.46E−01 | FALSE |
| Rras2 | 0.450 | 6.68E−03 | −0.666 | 8.71E−01 | 0.120 | 1.00E+00 | FALSE |
| Naca | 0.143 | 6.88E−03 | 0.104 | 1.00E+00 | 0.081 | 1.00E+00 | FALSE |
| Rps4x | 0.084 | 5.61E−02 | 0.223 | 7.11E−01 | 0.200 | 6.95E−03 | FALSE |
| Arl6ip1 | −0.237 | 7.04E−03 | −0.085 | 1.00E+00 | 0.120 | 1.00E+00 | FALSE |
| Fabp5 | −0.413 | 7.29E−03 | 0.290 | 1.00E+00 | −0.114 | 1.00E+00 | FALSE |
| Bhlhe40 | −0.178 | 7.29E−03 | 0.584 | 9.15E−01 | 0.150 | 9.46E−01 | FALSE |
| Ifi27l2a | −0.453 | 7.29E−03 | −0.400 | 1.00E+00 | −0.400 | 5.03E−01 | FALSE |
| Gch1 | −0.354 | 7.34E−03 | −0.211 | 1.00E+00 | −0.177 | 1.00E+00 | FALSE |
| Pdcd6ip | 0.333 | 7.47E−03 | 0.094 | 1.00E+00 | 0.163 | 1.00E+00 | FALSE |
| Tmem30a | 0.354 | 7.50E−03 | 0.730 | 6.80E−01 | −0.044 | 1.00E+00 | FALSE |
| Nkiras2 | 0.385 | 7.50E−03 | 0.432 | 9.94E−01 | −0.114 | 1.00E+00 | FALSE |
| mt-Nd4 | −0.178 | 7.50E−03 | −0.397 | 6.32E−01 | −0.183 | 7.35E−01 | FALSE |
| 5730508B09Rik | 0.411 | 7.56E−03 | 0.289 | 1.00E+00 | 0.126 | 1.00E+00 | FALSE |
| Myl6 | −0.135 | 7.97E−03 | −0.218 | 9.15E−01 | −0.269 | 3.29E−02 | FALSE |
| Atp5e | −0.169 | 8.21E−03 | −0.317 | 9.14E−01 | −0.217 | 4.85E−01 | FALSE |
| Wdr1 | 0.295 | 8.31E−03 | −0.266 | 1.00E+00 | 0.122 | 1.00E+00 | FALSE |
| Atp5c1 | 0.206 | 8.34E−03 | 0.374 | 8.76E−01 | 0.219 | 7.35E−01 | FALSE |
| Itgb1 | −0.468 | 8.89E−03 | −0.618 | 8.72E−01 | 0.090 | 1.00E+00 | FALSE |
| Cox5b | 0.196 | 9.36E−03 | 0.240 | 1.00E+00 | −0.081 | 1.00E+00 | FALSE |
| Serpinb9 | 0.353 | 9.55E−03 | −0.480 | 9.50E−01 | 0.299 | 8.20E−01 | FALSE |
| Plcxd2 | 0.420 | 9.75E−03 | 0.231 | 1.00E+00 | 0.296 | 8.28E−01 | FALSE |
| Lmnb1 | −0.311 | 9.75E−03 | −0.390 | 1.00E+00 | −0.461 | 5.14E−02 | FALSE |
| Adrbk1 | 0.381 | 9.85E−03 | −0.165 | 1.00E+00 | 0.340 | 6.97E−01 | FALSE |
| Nop56 | −0.353 | 9.85E−03 | −0.093 | 1.00E+00 | −0.215 | 9.46E−01 | FALSE |
| Cox6a1 | −0.185 | 1.01E−02 | −0.139 | 1.00E+00 | −0.212 | 6.21E−01 | FALSE |
| Eif3e | 0.189 | 1.05E−02 | 0.450 | 6.82E−01 | 0.126 | 9.63E−01 | FALSE |
| Pabpc1 | 0.143 | 1.07E−02 | 0.174 | 1.00E+00 | 0.076 | 1.00E+00 | FALSE |
| Ier5 | −0.307 | 1.09E−02 | −0.209 | 1.00E+00 | −0.233 | 9.46E−01 | FALSE |
| Ywhae | 0.259 | 1.09E−02 | 0.123 | 1.00E+00 | −0.257 | 7.35E−01 | FALSE |
| Ndufs2 | 0.339 | 1.09E−02 | 0.051 | 1.00E+00 | 0.250 | 8.90E−01 | FALSE |
| Gpr87 | 0.190 | 1.10E−02 | 0.000 | 1.00E+00 | 0.049 | 7.35E−01 | FALSE |
| Tox | −0.224 | 1.14E−02 | −0.463 | 8.31E−01 | 0.143 | 1.00E+00 | FALSE |
| Dnajb6 | −0.216 | 1.16E−02 | −0.168 | 1.00E+00 | −0.067 | 1.00E+00 | FALSE |
| Atp8a2 | 0.389 | 1.16E−02 | 0.651 | 8.72E−01 | 0.250 | 8.07E−01 | FALSE |
| Upf3a | 0.388 | 1.19E−02 | −0.135 | 1.00E+00 | 0.269 | 8.90E−01 | FALSE |
| Qrfp | 0.330 | 1.20E−02 | 0.048 | 1.00E+00 | 0.024 | 1.00E+00 | FALSE |
| PhIda1 | −0.420 | 1.21E−02 | −1.539 | 1.35E−02 | −0.750 | 3.16E−02 | FALSE |
| Mta3 | 0.395 | 1.22E−02 | 0.655 | 8.76E−01 | 0.344 | 7.29E−01 | FALSE |
| Kcnk5 | −0.422 | 1.24E−02 | 0.004 | 1.00E+00 | −0.163 | 1.00E+00 | FALSE |
| Myo1g | 0.320 | 1.24E−02 | 0.229 | 1.00E+00 | 0.108 | 1.00E+00 | FALSE |
| Leprotl1 | 0.262 | 1.27E−02 | −0.297 | 1.00E+00 | 0.065 | 1.00E+00 | FALSE |
| Cbfa2t3 | −0.182 | 1.29E−02 | 0.000 | 1.00E+00 | 0.000 | 1.00E+00 | FALSE |
| Fis1 | 0.262 | 1.32E−02 | 0.128 | 1.00E+00 | 0.027 | 1.00E+00 | FALSE |
| Gpx1 | −0.347 | 1.39E−02 | −0.165 | 1.00E+00 | −0.418 | 3.81E−01 | FALSE |
| Ctla4 | −0.212 | 1.36E−01 | −0.682 | 8.76E−01 | −0.553 | 1.42E−02 | FALSE |
| Bptf | −0.374 | 1.45E−02 | −0.037 | 1.00E+00 | −0.242 | 9.46E−01 | FALSE |
| Rplp2 | −0.097 | 1.45E−02 | −0.067 | 1.00E+00 | 0.169 | 8.47E−02 | FALSE |
| S1pr4 | 0.281 | 1.55E−02 | 0.045 | 1.00E+00 | 0.322 | 5.96E−01 | FALSE |
| Ndufa10 | 0.312 | 1.56E−02 | −0.009 | 1.00E+00 | −0.005 | 1.00E+00 | FALSE |
| Zfp706 | 0.270 | 1.59E−02 | −0.057 | 1.00E+00 | −0.138 | 1.00E+00 | FALSE |
| Lcn4 | 0.424 | 1.59E−02 | −0.167 | 1.00E+00 | 0.295 | 5.47E−01 | FALSE |
| Ndufa5 | −0.315 | 1.60E−02 | −0.163 | 1.00E+00 | −0.082 | 1.00E+00 | FALSE |
| Hnrnpf | 0.157 | 1.61E−02 | 0.229 | 9.64E−01 | 0.005 | 1.00E+00 | FALSE |
| Pglyrp1 | 0.344 | 1.61E−02 | 0.297 | 1.00E+00 | 0.190 | 1.00E+00 | FALSE |
| Ccni | 0.337 | 1.61E−02 | 0.026 | 1.00E+00 | 0.053 | 1.00E+00 | FALSE |
| Usp28 | −0.102 | 1.65E−02 | 0.000 | 1.00E+00 | −0.008 | 1.00E+00 | FALSE |
| Adrb2 | −0.424 | 1.65E−02 | 0.065 | 1.00E+00 | −0.173 | 1.00E+00 | FALSE |
| Cox7c | −0.160 | 1.65E−02 | −0.563 | 2.25E−02 | −0.196 | 5.04E−01 | FALSE |
| Klra3 | 0.342 | 1.66E−02 | 0.176 | 1.00E+00 | 0.000 | 1.00E+00 | FALSE |
| Traf1 | −0.207 | 1.67E−02 | −0.224 | 1.00E+00 | 0.048 | 1.00E+00 | FALSE |
| Idi1 | −0.396 | 1.67E−02 | 0.034 | 1.00E+00 | −0.128 | 1.00E+00 | FALSE |
| Capzb | 0.178 | 1.67E−02 | −0.097 | 1.00E+00 | −0.183 | 7.35E−01 | FALSE |
| Jtb | 0.329 | 1.68E−02 | −0.142 | 1.00E+00 | −0.236 | 9.46E−01 | FALSE |
| Tiparp | −0.384 | 1.70E−02 | 0.238 | 1.00E+00 | −0.154 | 1.00E+00 | FALSE |
| Arpp19 | −0.196 | 1.71E−02 | −0.612 | 1.15E−01 | −0.077 | 1.00E+00 | FALSE |
| Creg1 | −0.356 | 1.77E−02 | −0.250 | 1.00E+00 | −0.091 | 1.00E+00 | FALSE |
| Ten1 | 0.365 | 1.77E−02 | 0.388 | 1.00E+00 | 0.065 | 1.00E+00 | FALSE |
| Grcc10 | 0.340 | 1.81E−02 | −0.682 | 5.39E−01 | 0.383 | 5.47E−01 | FALSE |
| Grk6 | 0.312 | 1.82E−02 | 0.307 | 1.00E+00 | 0.284 | 7.35E−01 | FALSE |
| 2210011C24Rik | −0.347 | 1.84E−02 | −0.271 | 1.00E+00 | −0.095 | 1.00E+00 | FALSE |
| Racgap1 | 0.399 | 1.87E−02 | 0.222 | 1.00E+00 | 0.518 | 1.15E−01 | FALSE |
| Bcl3 | 0.382 | 1.92E−02 | 0.215 | 1.00E+00 | 0.069 | 1.00E+00 | FALSE |
| Slc7a5 | −0.363 | 1.92E−02 | 0.493 | 6.82E−01 | −0.261 | 9.46E−01 | FALSE |
| Rnf44 | 0.380 | 1.93E−02 | 0.634 | 7.11E−01 | −0.204 | 9.97E−01 | FALSE |
| Rcn2 | −0.368 | 1.94E−02 | −0.171 | 1.00E+00 | −0.038 | 1.00E+00 | FALSE |
| G3bp2 | −0.325 | 1.94E−02 | 0.028 | 1.00E+00 | −0.415 | 2.68E−01 | FALSE |
| Arf6 | −0.223 | 1.95E−02 | 0.089 | 1.00E+00 | −0.014 | 1.00E+00 | FALSE |
| Sqstm1 | −0.262 | 1.95E−02 | 0.318 | 1.00E+00 | −0.488 | 8.47E−02 | FALSE |
| Cmtm7 | 0.296 | 1.97E−02 | 1.027 | 8.51E−02 | 0.302 | 6.14E−01 | FALSE |
| Rhof | −0.357 | 2.01E−02 | 0.368 | 1.00E+00 | −0.245 | 9.46E−01 | FALSE |
| Atp1b3 | 0.309 | 2.02E−02 | −0.247 | 1.00E+00 | 0.171 | 1.00E+00 | FALSE |
| H2afx | −0.371 | 2.02E−02 | 0.061 | 1.00E+00 | 0.075 | 1.00E+00 | FALSE |
| Tmem176a | −0.510 | 2.04E−02 | −0.984 | 5.60E−01 | 0.024 | 1.00E+00 | FALSE |
| Tiprl | −0.311 | 2.06E−02 | 0.187 | 1.00E+00 | −0.049 | 1.00E+00 | FALSE |
| Gla | −0.349 | 2.10E−02 | 0.000 | 1.00E+00 | 0.021 | 1.00E+00 | FALSE |
| Mgst2 | 0.388 | 2.10E−02 | −0.115 | 1.00E+00 | −0.022 | 1.00E+00 | FALSE |
| Lgals3 | −0.235 | 2.11E−02 | −0.463 | 9.15E−01 | 0.243 | 9.55E−01 | FALSE |
| Rgs3 | 0.392 | 2.11E−02 | −0.055 | 1.00E+00 | −0.033 | 1.00E+00 | FALSE |
| Pttg1 | −0.339 | 2.11E−02 | −0.324 | 1.00E+00 | −0.268 | 7.97E−01 | FALSE |
| Ncald | 0.356 | 2.13E−02 | 0.427 | 1.00E+00 | −0.122 | 1.00E+00 | FALSE |
| Gadd45b | −0.198 | 4.60E−01 | 0.177 | 1.00E+00 | −0.729 | 2.24E−02 | FALSE |
| Malt1 | 0.138 | 7.69E−01 | −0.002 | 1.00E+00 | 0.595 | 2.26E−02 | FALSE |
| Dgat1 | −0.058 | 9.75E−01 | −0.018 | 1.00E+00 | −0.636 | 2.27E−02 | FALSE |
| Ubxn1 | 0.282 | 2.27E−02 | −0.490 | 9.15E−01 | 0.257 | 7.41E−01 | FALSE |
| Minos1 | −0.224 | 2.30E−02 | −0.557 | 4.66E−01 | 0.016 | 1.00E+00 | FALSE |
| Dctpp1 | −0.347 | 2.43E−02 | −0.144 | 1.00E+00 | −0.639 | 3.29E−02 | FALSE |
| Arrb2 | 0.318 | 2.45E−02 | 0.438 | 9.15E−01 | 0.178 | 1.00E+00 | FALSE |
| Ifngr1 | 0.031 | 9.32E−01 | −0.282 | 1.00E+00 | 0.312 | 2.50E−02 | FALSE |
| Mcpt1 | −0.103 | 2.50E−02 | −0.176 | 9.15E−01 | 0.000 | 1.00E+00 | FALSE |
| Kcnab2 | 0.335 | 2.61E−02 | −0.066 | 1.00E+00 | 0.257 | 9.26E−01 | FALSE |
| Nme1 | −0.256 | 2.61E−02 | −0.026 | 1.00E+00 | −0.321 | 6.24E−01 | FALSE |
| Ehd3 | 0.385 | 2.62E−02 | −0.101 | 1.00E+00 | 0.040 | 1.00E+00 | FALSE |
| Slbp | −0.286 | 2.62E−02 | −0.230 | 1.00E+00 | −0.194 | 9.46E−01 | FALSE |
| Sparc | −0.187 | 2.63E−02 | 0.000 | 1.00E+00 | −0.042 | 1.00E+00 | FALSE |
| Ywhab | 0.282 | 2.72E−02 | −0.069 | 1.00E+00 | 0.027 | 1.00E+00 | FALSE |
| Umps | −0.356 | 2.75E−02 | 0.090 | 1.00E+00 | 0.140 | 1.00E+00 | FALSE |
| Zfand2b | 0.348 | 2.78E−02 | −0.257 | 1.00E+00 | 0.097 | 1.00E+00 | FALSE |
| Vamp4 | 0.345 | 2.78E−02 | −0.247 | 1.00E+00 | −0.037 | 1.00E+00 | FALSE |
| Pcgf1 | 0.323 | 2.78E−02 | 0.091 | 1.00E+00 | −0.006 | 1.00E+00 | FALSE |
| Srgap3 | −0.127 | 2.79E−02 | 0.000 | 1.00E+00 | −0.087 | 7.97E−01 | FALSE |
| H2-DMa | 0.363 | 2.83E−02 | −0.277 | 1.00E+00 | −0.222 | 9.46E−01 | FALSE |
| Dkkl1 | −0.357 | 2.83E−02 | −0.728 | 7.11E−01 | −0.088 | 1.00E+00 | FALSE |
| Camk2n1 | −0.374 | 2.84E−02 | 0.020 | 1.00E+00 | −0.148 | 1.00E+00 | FALSE |
| Dnajb9 | −0.335 | 2.84E−02 | −0.029 | 1.00E+00 | −0.028 | 1.00E+00 | FALSE |
| Irf2 | 0.347 | 2.86E−02 | 0.340 | 1.00E+00 | 0.224 | 9.01E−01 | FALSE |
| Gas7 | −0.410 | 2.91E−02 | −0.252 | 1.00E+00 | −0.095 | 1.00E+00 | FALSE |
| Eef1a1 | 0.015 | 9.92E−01 | 0.083 | 1.00E+00 | 0.160 | 2.91E−02 | FALSE |
| Dtx1 | 0.383 | 2.92E−02 | 1.064 | 2.57E−01 | 0.003 | 1.00E+00 | FALSE |
| Tra2b | −0.246 | 2.98E−02 | −0.198 | 1.00E+00 | −0.211 | 7.41E−01 | FALSE |
| Zfp207 | −0.006 | 1.00E+00 | 1.181 | 3.02E−02 | 0.121 | 1.00E+00 | FALSE |
| Wac | −0.329 | 3.02E−02 | 0.143 | 1.00E+00 | −0.013 | 1.00E+00 | FALSE |
| H2-Q10 | −0.344 | 3.05E−02 | 0.052 | 1.00E+00 | −0.276 | 9.46E−01 | FALSE |
| Ino80b | 0.357 | 3.06E−02 | 0.345 | 1.00E+00 | 0.163 | 1.00E+00 | FALSE |
| Dnajb2 | −0.299 | 3.06E−02 | −0.203 | 1.00E+00 | −0.143 | 1.00E+00 | FALSE |
| Abhd2 | −0.391 | 3.07E−02 | 0.058 | 1.00E+00 | −0.104 | 1.00E+00 | FALSE |
| Hmha1 | 0.213 | 3.07E−02 | −0.187 | 1.00E+00 | 0.225 | 7.12E−01 | FALSE |
| Rbm39 | −0.145 | 3.08E−02 | 0.144 | 1.00E+00 | −0.057 | 1.00E+00 | FALSE |
| Cox17 | −0.225 | 3.08E−02 | −0.523 | 4.52E−01 | −0.216 | 7.61E−01 | FALSE |
| Lpcat4 | 0.358 | 3.10E−02 | 0.324 | 9.15E−01 | 0.187 | 9.97E−01 | FALSE |
| Rps3 | 0.083 | 3.10E−02 | 0.221 | 6.98E−01 | 0.147 | 6.94E−02 | FALSE |
| Eef1b2 | 0.105 | 4.60E−01 | 0.269 | 1.00E+00 | 0.352 | 3.11E−02 | FALSE |
| Pltp | 0.026 | 1.00E+00 | −1.002 | 3.15E−02 | 0.104 | 1.00E+00 | FALSE |
| Pcbd2 | −0.363 | 3.18E−02 | −0.327 | 1.00E+00 | −0.114 | 1.00E+00 | FALSE |
| Eif3h | 0.148 | 3.18E−02 | 0.241 | 9.75E−01 | 0.194 | 6.12E−01 | FALSE |
| Fam103a1 | 0.235 | 3.20E−02 | 0.525 | 8.76E−01 | −0.045 | 1.00E+00 | FALSE |
| H2-Ke6 | −0.334 | 3.20E−02 | 0.192 | 1.00E+00 | 0.039 | 1.00E+00 | FALSE |
| Ube2v1 | 0.305 | 3.32E−02 | −0.059 | 1.00E+00 | 0.246 | 8.36E−01 | FALSE |
| Tbc1d22a | 0.117 | 7.96E−01 | −1.021 | 3.39E−02 | 0.012 | 1.00E+00 | FALSE |
| Gimap6 | 0.179 | 3.39E−02 | 0.096 | 1.00E+00 | −0.086 | 1.00E+00 | FALSE |
| Mien1 | −0.260 | 3.43E−02 | −0.462 | 9.15E−01 | −0.345 | 5.06E−01 | FALSE |
| Cbwd1 | 0.228 | 3.43E−02 | 0.007 | 1.00E+00 | 0.168 | 9.56E−01 | FALSE |
| Kxd1 | 0.305 | 3.43E−02 | 0.388 | 1.00E+00 | 0.295 | 7.35E−01 | FALSE |
| Rpl39 | 0.101 | 3.43E−02 | 0.185 | 9.15E−01 | 0.134 | 4.67E−01 | FALSE |
| Dedd2 | −0.264 | 1.92E−01 | −0.225 | 1.00E+00 | −0.657 | 3.47E−02 | FALSE |
| Tbc1d17 | 0.340 | 3.50E−02 | 0.289 | 1.00E+00 | 0.240 | 9.46E−01 | FALSE |
| Rpl23a | −0.080 | 3.50E−02 | 0.004 | 1.00E+00 | 0.100 | 5.96E−01 | FALSE |
| Coro1b | 0.310 | 3.50E−02 | 0.281 | 1.00E+00 | 0.261 | 8.78E−01 | FALSE |
| Tmem71 | 0.354 | 3.62E−02 | −0.056 | 1.00E+00 | 0.410 | 5.04E−01 | FALSE |
| Tmem9b | 0.291 | 3.64E−02 | 0.253 | 1.00E+00 | 0.108 | 1.00E+00 | FALSE |
| S100a6 | −0.059 | 8.25E−01 | −0.248 | 1.00E+00 | −0.564 | 3.72E−02 | FALSE |
| Fam49b | 0.219 | 3.75E−02 | 0.090 | 1.00E+00 | −0.029 | 1.00E+00 | FALSE |
| Fundc2 | 0.288 | 3.77E−02 | 0.126 | 1.00E+00 | 0.407 | 3.32E−01 | FALSE |
| Smim24 | 0.243 | 3.77E−02 | −0.154 | 9.15E−01 | 0.055 | 1.00E+00 | FALSE |
| Pdia6 | −0.296 | 3.79E−02 | −0.657 | 7.11E−01 | −0.255 | 8.90E−01 | FALSE |
| Klrc1 | −0.278 | 3.82E−02 | −0.712 | 8.76E−01 | 0.430 | 7.35E−01 | FALSE |
| Pelp1 | −0.266 | 3.85E−02 | −0.372 | 9.96E−01 | −0.099 | 1.00E+00 | FALSE |
| Ncl | −0.195 | 3.85E−02 | 0.012 | 1.00E+00 | −0.067 | 1.00E+00 | FALSE |
| Efcab7 | 0.106 | 3.88E−02 | 0.055 | 1.00E+00 | −0.062 | 1.00E+00 | FALSE |
| Arl4a | −0.350 | 3.88E−02 | 0.488 | 9.15E−01 | −0.074 | 1.00E+00 | FALSE |
| Id3 | −0.404 | 3.89E−02 | −0.430 | 1.00E+00 | 0.036 | 1.00E+00 | FALSE |
| Gm27166 | −0.090 | 3.95E−02 | −0.218 | 1.00E+00 | 0.000 | 1.00E+00 | FALSE |
| Tnfaip8 | −0.284 | 3.97E−02 | −0.300 | 1.00E+00 | 0.123 | 1.00E+00 | FALSE |
| Rps26 | 0.132 | 4.28E−02 | 0.146 | 1.00E+00 | −0.003 | 1.00E+00 | FALSE |
| Map2k3 | −0.280 | 4.28E−02 | −0.009 | 1.00E+00 | −0.411 | 4.91E−01 | FALSE |
| Pfn1 | 0.098 | 4.30E−02 | −0.050 | 1.00E+00 | −0.053 | 1.00E+00 | FALSE |
| Acoxl | 0.266 | 4.33E−02 | 0.135 | 1.00E+00 | −0.180 | 9.46E−01 | FALSE |
| Cxcl10 | −0.504 | 4.35E−02 | −0.424 | 8.76E−01 | 0.135 | 1.00E+00 | FALSE |
| Mbd2 | −0.321 | 4.35E−02 | −0.438 | 1.00E+00 | −0.068 | 1.00E+00 | FALSE |
| Fcrls | −0.105 | 4.35E−02 | 0.064 | 1.00E+00 | −0.074 | 9.46E−01 | FALSE |
| Arpc1b | 0.128 | 4.42E−02 | −0.038 | 1.00E+00 | −0.078 | 1.00E+00 | FALSE |
| Tomm7 | −0.200 | 4.43E−02 | −0.506 | 5.07E−01 | −0.005 | 1.00E+00 | FALSE |
| Idh2 | 0.319 | 4.43E−02 | 0.190 | 1.00E+00 | 0.073 | 1.00E+00 | FALSE |
| Gbp2 | 0.396 | 4.43E−02 | −0.357 | 1.00E+00 | −0.043 | 1.00E+00 | FALSE |
| Rpl28 | 0.143 | 4.43E−02 | 0.150 | 1.00E+00 | 0.112 | 9.46E−01 | FALSE |
| Pgk1 | 0.324 | 4.46E−02 | 0.573 | 8.76E−01 | 0.211 | 7.35E−01 | FALSE |
| Caprin1 | 0.343 | 4.49E−02 | 0.070 | 1.00E+00 | −0.007 | 1.00E+00 | FALSE |
| Rpl7 | 0.101 | 4.51E−02 | 0.178 | 9.96E−01 | 0.157 | 3.06E−01 | FALSE |
| Chmp6 | 0.336 | 4.53E−02 | 0.110 | 1.00E+00 | 0.248 | 9.40E−01 | FALSE |
| Mtx2 | −0.324 | 4.55E−02 | −0.280 | 1.00E+00 | 0.122 | 1.00E+00 | FALSE |
| Cmc1 | −0.009 | 1.00E+00 | −0.099 | 1.00E+00 | −0.605 | 4.62E−02 | FALSE |
| Gm4950 | 0.216 | 3.59E−01 | 0.474 | 9.96E−01 | 0.569 | 4.66E−02 | FALSE |
| Clic1 | 0.142 | 4.69E−02 | 0.072 | 1.00E+00 | 0.102 | 1.00E+00 | FALSE |
| Junb | −0.182 | 4.71E−02 | −0.519 | 7.11E−01 | −0.132 | 9.47E−01 | FALSE |
| Rasl11a | −0.452 | 4.71E−02 | 0.081 | 1.00E+00 | −0.054 | 1.00E+00 | FALSE |
| Celf2 | 0.236 | 4.81E−02 | 0.202 | 1.00E+00 | 0.116 | 1.00E+00 | FALSE |
| Rps15a | −0.038 | 6.52E−01 | 0.006 | 1.00E+00 | 0.153 | 4.90E−02 | FALSE |
| Mapkap1 | 0.130 | 7.72E−01 | −1.060 | 4.90E−02 | −0.216 | 1.00E+00 | FALSE |
| Man1a | −0.336 | 5.01E−02 | −0.184 | 1.00E+00 | −0.014 | 1.00E+00 | FALSE |
| Vcpip1 | −0.311 | 5.17E−02 | 0.086 | 1.00E+00 | −0.164 | 1.00E+00 | FALSE |
| Zfp280d | 0.083 | 9.31E−01 | 0.025 | 1.00E+00 | 0.564 | 5.20E−02 | FALSE |
| Pstpip1 | 0.278 | 5.28E−02 | 0.256 | 1.00E+00 | 0.157 | 1.00E+00 | FALSE |
| Gramd3 | 0.212 | 5.32E−02 | 0.502 | 9.26E−01 | 0.260 | 5.46E−01 | FALSE |
| Edaradd | −0.285 | 5.32E−02 | 0.033 | 1.00E+00 | −0.097 | 7.83E−01 | FALSE |
| Mrpl46 | −0.108 | 8.72E−01 | −0.102 | 1.00E+00 | −0.596 | 5.35E−02 | FALSE |
| Ecm1 | −0.279 | 5.39E−02 | −0.297 | 1.00E+00 | −0.129 | 1.00E+00 | FALSE |
| Gmfb | 0.328 | 5.39E−02 | 0.229 | 1.00E+00 | −0.132 | 1.00E+00 | FALSE |
| Rps16 | −0.068 | 1.32E−01 | −0.051 | 1.00E+00 | 0.154 | 5.42E−02 | FALSE |
| Atp6v0e | −0.084 | 6.84E−01 | −0.439 | 7.11E−01 | −0.408 | 5.42E−02 | FALSE |
| Ppp2ca | −0.214 | 5.53E−02 | −0.178 | 1.00E+00 | −0.029 | 1.00E+00 | FALSE |
| Spint2 | −0.307 | 5.56E−02 | −0.062 | 1.00E+00 | −0.036 | 1.00E+00 | FALSE |
| Gm10031 | 0.188 | 5.60E−02 | 0.051 | 1.00E+00 | 0.033 | 1.00E+00 | FALSE |
| Fam134b | 0.334 | 5.61E−02 | −0.002 | 1.00E+00 | 0.052 | 1.00E+00 | FALSE |
| Wdr45b | −0.324 | 5.61E−02 | −1.048 | 1.03E−01 | 0.043 | 1.00E+00 | FALSE |
| Lypla1 | 0.291 | 5.63E−02 | 0.042 | 1.00E+00 | 0.041 | 1.00E+00 | FALSE |
| B4galnt4 | 0.211 | 5.68E−02 | 0.102 | 1.00E+00 | −0.115 | 7.35E−01 | FALSE |
| Gstt2 | 0.331 | 5.77E−02 | 0.544 | 9.15E−01 | −0.080 | 1.00E+00 | FALSE |
| Sh2d2a | 0.242 | 5.78E−02 | 0.730 | 5.39E−01 | 0.125 | 1.00E+00 | FALSE |
| Mllt3 | −0.024 | 1.00E+00 | −1.062 | 5.81E−02 | 0.144 | 1.00E+00 | FALSE |
| H2-DMb1 | 0.138 | 5.85E−02 | −0.045 | 1.00E+00 | −0.032 | 1.00E+00 | FALSE |
| BC055324 | −0.090 | 5.85E−02 | 0.000 | 1.00E+00 | 0.094 | 7.35E−01 | FALSE |
| Csrnp1 | −0.331 | 5.85E−02 | 0.202 | 1.00E+00 | −0.254 | 9.40E−01 | FALSE |
| Zyg11b | 0.329 | 5.85E−02 | 0.333 | 1.00E+00 | −0.155 | 1.00E+00 | FALSE |
| Hadha | 0.321 | 5.85E−02 | 0.074 | 1.00E+00 | −0.224 | 9.61E−01 | FALSE |
| Dnaja4 | −0.385 | 5.87E−02 | −0.358 | 1.00E+00 | −0.514 | 3.37E−01 | FALSE |
| Abcg2 | −0.115 | 5.89E−02 | 0.000 | 1.00E+00 | −0.043 | 1.00E+00 | FALSE |
| Unc119b | 0.327 | 5.90E−02 | 0.779 | 6.82E−01 | −0.031 | 1.00E+00 | FALSE |
| Ablim1 | 0.278 | 5.90E−02 | −0.098 | 1.00E+00 | −0.153 | 1.00E+00 | FALSE |
| Zmym2 | −0.045 | 1.00E+00 | 0.119 | 1.00E+00 | 0.465 | 5.96E−02 | FALSE |
| Ncf1 | 0.280 | 5.96E−02 | 0.136 | 1.00E+00 | 0.163 | 7.21E−01 | FALSE |
| Tomm22 | 0.220 | 5.97E−02 | 0.135 | 1.00E+00 | 0.040 | 1.00E+00 | FALSE |
| Srsf5 | −0.179 | 5.98E−02 | 0.235 | 1.00E+00 | −0.080 | 1.00E+00 | FALSE |
| Plin2 | −0.345 | 5.99E−02 | −0.147 | 1.00E+00 | −0.199 | 9.84E−01 | FALSE |
| Tlk1 | 0.321 | 5.99E−02 | 0.084 | 1.00E+00 | 0.095 | 1.00E+00 | FALSE |
| Cenpl | −0.337 | 5.99E−02 | −0.048 | 1.00E+00 | −0.152 | 1.00E+00 | FALSE |
| Galnt3 | −0.232 | 6.00E−02 | 0.000 | 1.00E+00 | −0.059 | 9.46E−01 | FALSE |
| Gm16286 | 0.160 | 5.25E−01 | 0.064 | 1.00E+00 | −0.520 | 6.01E−02 | FALSE |
| Ctnna1 | −0.336 | 6.03E−02 | −0.153 | 1.00E+00 | 0.048 | 1.00E+00 | FALSE |
| Ift52 | 0.337 | 6.05E−02 | 0.630 | 8.76E−01 | −0.119 | 1.00E+00 | FALSE |
| Alg6 | −0.185 | 6.08E−02 | −0.153 | 1.00E+00 | 0.014 | 1.00E+00 | FALSE |
| Orc4 | 0.330 | 6.08E−02 | −0.195 | 1.00E+00 | 0.140 | 1.00E+00 | FALSE |
| Otud1 | −0.266 | 6.17E−02 | 0.000 | 1.00E+00 | 0.036 | 1.00E+00 | FALSE |
| Ipo11 | −0.273 | 6.20E−02 | −0.683 | 2.94E−01 | −0.156 | 9.77E−01 | FALSE |
| Gm12216 | 0.333 | 6.20E−02 | −0.090 | 1.00E+00 | 0.204 | 1.00E+00 | FALSE |
| D3Ertd751e | −0.267 | 6.20E−02 | −0.170 | 1.00E+00 | −0.156 | 1.00E+00 | FALSE |
| Tnfrsf13c | 0.174 | 6.20E−02 | 0.051 | 1.00E+00 | 0.014 | 1.00E+00 | FALSE |
| Hist1h1e | 0.316 | 6.21E−02 | 0.546 | 6.92E−01 | 0.235 | 6.69E−01 | FALSE |
| Emp1 | −0.334 | 6.44E−02 | −0.323 | 1.00E+00 | 0.118 | 1.00E+00 | FALSE |
| Pcbp2 | 0.180 | 6.44E−02 | 0.128 | 1.00E+00 | 0.016 | 1.00E+00 | FALSE |
| Tspan3 | 0.337 | 6.51E−02 | 0.000 | 1.00E+00 | 0.270 | 7.96E−01 | FALSE |
| Xpa | 0.315 | 6.52E−02 | 0.152 | 1.00E+00 | 0.172 | 1.00E+00 | FALSE |
| Tmem173 | 0.320 | 6.52E−02 | −0.293 | 1.00E+00 | −0.230 | 9.46E−01 | FALSE |
| Ndufa4 | 0.236 | 6.53E−02 | 0.088 | 1.00E+00 | −0.274 | 4.85E−01 | FALSE |
| Mrto4 | −0.325 | 6.54E−02 | −0.203 | 1.00E+00 | −0.130 | 1.00E+00 | FALSE |
| Zfp277 | 0.321 | 6.64E−02 | 0.146 | 1.00E+00 | 0.274 | 8.15E−01 | FALSE |
| Nrp1 | −0.342 | 6.68E−02 | 0.285 | 8.76E−01 | 0.057 | 7.35E−01 | FALSE |
| Yme1l1 | −0.313 | 6.68E−02 | −0.138 | 1.00E+00 | −0.183 | 1.00E+00 | FALSE |
| Eva1b | 0.313 | 6.72E−02 | 0.297 | 1.00E+00 | 0.073 | 1.00E+00 | FALSE |
| Pkp4 | 0.176 | 6.72E−02 | −0.113 | 1.00E+00 | 0.002 | 1.00E+00 | FALSE |
| Etfb | −0.269 | 6.77E−02 | −0.375 | 9.96E−01 | −0.039 | 1.00E+00 | FALSE |
| Psmc2 | −0.280 | 6.77E−02 | 0.033 | 1.00E+00 | −0.148 | 1.00E+00 | FALSE |
| Fmnl1 | 0.284 | 6.82E−02 | 0.332 | 1.00E+00 | 0.254 | 8.90E−01 | FALSE |
| Ddit4 | −0.349 | 6.85E−02 | −0.244 | 1.00E+00 | −0.132 | 1.00E+00 | FALSE |
| Atpif1 | −0.254 | 6.92E−02 | −0.100 | 1.00E+00 | 0.157 | 1.00E+00 | FALSE |
| Uqcrh | 0.127 | 6.93E−02 | 0.100 | 1.00E+00 | −0.025 | 1.00E+00 | FALSE |
| Sfpq | 0.230 | 6.95E−02 | 0.870 | 2.05E−01 | −0.057 | 1.00E+00 | FALSE |
| Psmb10 | 0.243 | 6.95E−02 | −0.180 | 1.00E+00 | 0.238 | 8.20E−01 | FALSE |
| Rab5c | −0.261 | 6.96E−02 | −0.087 | 1.00E+00 | −0.038 | 1.00E+00 | FALSE |
| Phf3 | −0.305 | 6.96E−02 | 0.196 | 1.00E+00 | −0.109 | 1.00E+00 | FALSE |
| Dusp5 | −0.200 | 6.98E−02 | −0.086 | 1.00E+00 | −0.135 | 1.00E+00 | FALSE |
| Psmb1 | 0.145 | 6.98E−02 | 0.181 | 1.00E+00 | 0.144 | 8.93E−01 | FALSE |
| Mrpl43 | 0.282 | 6.98E−02 | −0.374 | 1.00E+00 | −0.221 | 9.57E−01 | FALSE |
| Dusp12 | 0.086 | 9.05E−01 | −0.202 | 1.00E+00 | 0.547 | 6.99E−02 | FALSE |
| Rpn2 | −0.264 | 7.05E−02 | −0.278 | 1.00E+00 | −0.187 | 9.77E−01 | FALSE |
| Magohb | 0.328 | 7.05E−02 | 0.218 | 1.00E+00 | −0.065 | 1.00E+00 | FALSE |
| Pcna | −0.291 | 7.34E−02 | −0.023 | 1.00E+00 | −0.035 | 1.00E+00 | FALSE |
| Nrros | −0.379 | 7.36E−02 | −0.237 | 1.00E+00 | 0.068 | 1.00E+00 | FALSE |
| Banf1 | −0.297 | 7.43E−02 | 0.297 | 1.00E+00 | −0.039 | 1.00E+00 | FALSE |
| Zfp40 | 0.101 | 7.43E−02 | −0.176 | 9.15E−01 | 0.048 | 9.46E−01 | FALSE |
| Tmem109 | −0.320 | 7.47E−02 | −0.005 | 1.00E+00 | −0.051 | 1.00E+00 | FALSE |
| Lbh | 0.297 | 7.49E−02 | 0.253 | 1.00E+00 | −0.370 | 6.10E−01 | FALSE |
| Surf1 | 0.304 | 7.50E−02 | 0.341 | 1.00E+00 | 0.176 | 1.00E+00 | FALSE |
| Ppp1ca | 0.139 | 7.50E−02 | −0.055 | 1.00E+00 | −0.015 | 1.00E+00 | FALSE |
| Arid4b | −0.315 | 7.50E−02 | −0.382 | 1.00E+00 | 0.147 | 1.00E+00 | FALSE |
| Akr1a1 | 0.223 | 7.50E−02 | 0.014 | 1.00E+00 | 0.033 | 1.00E+00 | FALSE |
| Ctsb | −0.040 | 1.00E+00 | −0.149 | 1.00E+00 | −0.485 | 7.51E−02 | FALSE |
| Amd1 | −0.337 | 7.59E−02 | −0.231 | 1.00E+00 | −0.155 | 1.00E+00 | FALSE |
| Tulp3 | 0.134 | 7.60E−02 | 0.058 | 1.00E+00 | −0.043 | 1.00E+00 | FALSE |
| Hsd17b7 | −0.260 | 7.72E−02 | −0.222 | 1.00E+00 | −0.024 | 1.00E+00 | FALSE |
| Nsun3 | 0.221 | 7.72E−02 | 0.333 | 1.00E+00 | 0.163 | 1.00E+00 | FALSE |
| Tgfbr1 | −0.276 | 7.74E−02 | 0.522 | 9.15E−01 | −0.067 | 1.00E+00 | FALSE |
| Lsm2 | −0.308 | 7.80E−02 | −0.321 | 1.00E+00 | −0.023 | 1.00E+00 | FALSE |
| Fam118b | 0.255 | 7.84E−02 | 0.261 | 1.00E+00 | 0.244 | 7.35E−01 | FALSE |
| Impact | −0.321 | 7.84E−02 | 0.441 | 1.00E+00 | −0.186 | 1.00E+00 | FALSE |
| Lamtor1 | 0.291 | 7.84E−02 | −0.066 | 1.00E+00 | −0.020 | 1.00E+00 | FALSE |
| R3hdm4 | 0.273 | 7.87E−02 | −0.385 | 1.00E+00 | −0.149 | 1.00E+00 | FALSE |
| Ilk | 0.286 | 7.88E−02 | −0.143 | 1.00E+00 | 0.310 | 7.35E−01 | FALSE |
| mt-Nd3 | −0.253 | 9.74E−02 | −0.472 | 9.15E−01 | −0.548 | 7.90E−02 | FALSE |
| Mdh1 | −0.024 | 1.00E+00 | −0.038 | 1.00E+00 | −0.428 | 7.90E−02 | FALSE |
| Rpl27 | 0.089 | 7.95E−02 | 0.094 | 1.00E+00 | 0.133 | 3.83E−01 | FALSE |
| Enc1 | −0.105 | 7.96E−02 | 0.059 | 1.00E+00 | −0.067 | 9.46E−01 | FALSE |
| Cd3d | 0.025 | 1.00E+00 | −0.443 | 7.99E−02 | −0.055 | 1.00E+00 | FALSE |
| Rnf2 | 0.314 | 8.02E−02 | 0.039 | 1.00E+00 | 0.139 | 1.00E+00 | FALSE |
| Rnf167 | −0.155 | 6.14E−01 | −0.996 | 8.03E−02 | 0.336 | 6.95E−01 | FALSE |
| Ptpn1 | −0.048 | 9.95E−01 | 0.347 | 1.00E+00 | −0.471 | 8.07E−02 | FALSE |
| Grhpr | 0.283 | 8.07E−02 | 0.106 | 1.00E+00 | 0.037 | 1.00E+00 | FALSE |
| Polr2f | −0.285 | 8.11E−02 | −0.614 | 8.71E−01 | 0.012 | 1.00E+00 | FALSE |
| Vcp | −0.107 | 6.84E−01 | −0.592 | 6.37E−01 | −0.475 | 8.19E−02 | FALSE |
| Atl2 | −0.280 | 8.20E−02 | 0.000 | 1.00E+00 | 0.188 | 1.00E+00 | FALSE |
| Etv5 | −0.078 | 8.20E−02 | 0.109 | 1.00E+00 | −0.018 | 1.00E+00 | FALSE |
| Postn | −0.077 | 8.20E−02 | 0.000 | 1.00E+00 | −0.022 | 1.00E+00 | FALSE |
| Gm18025 | 0.197 | 8.27E−02 | 0.000 | 1.00E+00 | 0.093 | 1.00E+00 | FALSE |
| Pot1a | 0.288 | 8.27E−02 | −0.438 | 9.15E−01 | 0.031 | 1.00E+00 | FALSE |
| Sdhb | 0.224 | 8.27E−02 | −0.064 | 1.00E+00 | 0.014 | 1.00E+00 | FALSE |
| Bmyc | −0.252 | 8.27E−02 | −0.049 | 1.00E+00 | 0.074 | 1.00E+00 | FALSE |
| Hamp | −0.072 | 8.27E−02 | 0.000 | 1.00E+00 | 0.000 | 1.00E+00 | FALSE |
| Btg1 | 0.124 | 1.23E−01 | 0.404 | 6.60E−01 | 0.229 | 8.31E−02 | FALSE |
| Il4 | 0.110 | 8.33E−02 | −0.335 | 7.11E−01 | 0.000 | 1.00E+00 | FALSE |
| Nrg4 | −0.070 | 8.33E−02 | −0.175 | 9.15E−01 | −0.011 | 1.00E+00 | FALSE |
| Eif2a | 0.296 | 8.33E−02 | 0.207 | 1.00E+00 | 0.297 | 7.42E−01 | FALSE |
| Xbp1 | −0.236 | 1.17E−01 | 0.226 | 1.00E+00 | −0.512 | 8.36E−02 | FALSE |
| Btg3 | 0.296 | 8.36E−02 | −0.102 | 1.00E+00 | 0.404 | 4.24E−01 | FALSE |
| Skap1 | −0.084 | 7.23E−01 | −0.735 | 8.40E−02 | 0.052 | 1.00E+00 | FALSE |
| Lysmd2 | 0.341 | 8.45E−02 | 0.142 | 1.00E+00 | −0.156 | 1.00E+00 | FALSE |
| Golm1 | 0.316 | 8.45E−02 | −0.067 | 1.00E+00 | 0.027 | 1.00E+00 | FALSE |
| 1700025G04Rik | 0.329 | 8.48E−02 | −0.016 | 1.00E+00 | 0.461 | 1.30E−01 | FALSE |
| Arf5 | 0.066 | 7.56E−01 | 0.088 | 1.00E+00 | −0.329 | 8.50E−02 | FALSE |
| Rpl9 | −0.078 | 8.62E−02 | 0.056 | 1.00E+00 | 0.032 | 1.00E+00 | FALSE |
| Thap3 | 0.316 | 8.66E−02 | 0.168 | 1.00E+00 | 0.162 | 1.00E+00 | FALSE |
| Hmgb3 | −0.281 | 8.66E−02 | 0.003 | 1.00E+00 | 0.093 | 1.00E+00 | FALSE |
| Sirt2 | 0.269 | 8.67E−02 | 0.180 | 1.00E+00 | −0.080 | 1.00E+00 | FALSE |
| Rbm8a2 | 0.307 | 8.67E−02 | 0.102 | 1.00E+00 | 0.086 | 1.00E+00 | FALSE |
| Elmsan1 | 0.171 | 5.18E−01 | 0.276 | 9.15E−01 | 0.478 | 8.68E−02 | FALSE |
| Gm12355 | 0.215 | 1.99E−01 | 0.329 | 8.80E−01 | 0.461 | 8.69E−02 | FALSE |
| Chst11 | 0.008 | 1.00E+00 | 0.199 | 1.00E+00 | −0.507 | 8.69E−02 | FALSE |
| Gm6576 | 0.212 | 8.76E−02 | 0.170 | 1.00E+00 | 0.201 | 8.03E−01 | FALSE |
| Rpe | 0.309 | 8.76E−02 | 0.388 | 1.00E+00 | 0.361 | 5.96E−01 | FALSE |
| Exph5 | −0.115 | 8.76E−02 | 0.059 | 1.00E+00 | 0.000 | 1.00E+00 | FALSE |
| Snx5 | 0.265 | 8.76E−02 | −0.061 | 1.00E+00 | −0.016 | 1.00E+00 | FALSE |
| Rpl35a | 0.035 | 7.86E−01 | 0.041 | 1.00E+00 | 0.175 | 8.83E−02 | FALSE |
| Aars | −0.293 | 8.93E−02 | −0.280 | 1.00E+00 | −0.119 | 1.00E+00 | FALSE |
| Rlim | −0.306 | 8.95E−02 | 0.121 | 1.00E+00 | −0.249 | 9.46E−01 | FALSE |
| Gm17056 | −0.239 | 8.96E−02 | 0.000 | 1.00E+00 | 0.080 | 1.00E+00 | FALSE |
| Zdhhc16 | 0.279 | 8.96E−02 | −0.225 | 1.00E+00 | 0.031 | 1.00E+00 | FALSE |
| Kras | 0.260 | 8.97E−02 | 0.619 | 9.15E−01 | −0.045 | 1.00E+00 | FALSE |
| Rnf7 | 0.268 | 8.97E−02 | −0.266 | 1.00E+00 | 0.129 | 1.00E+00 | FALSE |
| Casc4 | 0.109 | 8.97E−02 | 0.180 | 1.00E+00 | −0.067 | 1.00E+00 | FALSE |
| Ggt5 | 0.181 | 9.01E−02 | 0.333 | 1.00E+00 | 0.026 | 1.00E+00 | FALSE |
| Cks1brt | −0.077 | 9.01E−02 | 0.000 | 1.00E+00 | 0.000 | 1.00E+00 | FALSE |
| Taf1b | 0.308 | 9.01E−02 | −0.219 | 1.00E+00 | 0.114 | 1.00E+00 | FALSE |
| Tmed10 | −0.232 | 9.01E−02 | −0.182 | 1.00E+00 | −0.131 | 1.00E+00 | FALSE |
| Eif4e3 | 0.323 | 9.01E−02 | 0.310 | 1.00E+00 | 0.201 | 1.00E+00 | FALSE |
| Sh2d1a | 0.280 | 9.01E−02 | −0.024 | 1.00E+00 | 0.126 | 1.00E+00 | FALSE |
| Brd2 | −0.280 | 9.01E−02 | −0.274 | 1.00E+00 | −0.020 | 1.00E+00 | FALSE |
| Ruben | −0.152 | 9.01E−02 | 0.058 | 1.00E+00 | 0.035 | 1.00E+00 | FALSE |
| Arrdc1 | 0.297 | 9.01E−02 | 0.108 | 1.00E+00 | 0.098 | 1.00E+00 | FALSE |
| Fam46a | −0.371 | 9.01E−02 | −0.161 | 1.00E+00 | 0.079 | 1.00E+00 | FALSE |
| Neat1 | 0.270 | 9.01E−02 | 0.623 | 9.15E−01 | 0.504 | 9.18E−02 | FALSE |
| Mrps21 | 0.263 | 9.01E−02 | −0.402 | 9.31E−01 | 0.113 | 1.00E+00 | FALSE |
| 5330426L24Rik | 0.102 | 9.01E−02 | 0.169 | 1.00E+00 | 0.000 | 1.00E+00 | FALSE |
| Aprt | 0.225 | 9.01E−02 | −0.208 | 1.00E+00 | −0.072 | 1.00E+00 | FALSE |
| Hivep2 | 0.311 | 9.01E−02 | −0.315 | 1.00E+00 | 0.408 | 4.29E−01 | FALSE |
| Commd8 | 0.258 | 9.01E−02 | 0.346 | 1.00E+00 | 0.158 | 1.00E+00 | FALSE |
| Dcaf15 | 0.314 | 9.05E−02 | −0.192 | 1.00E+00 | 0.250 | 8.20E−01 | FALSE |
| Gm5426 | 0.146 | 9.14E−02 | −0.126 | 1.00E+00 | −0.044 | 1.00E+00 | FALSE |
| Zfos1 | 0.253 | 9.20E−02 | 0.560 | 9.15E−01 | −0.034 | 1.00E+00 | FALSE |
| Htatsf1 | −0.306 | 9.20E−02 | 0.455 | 9.15E−01 | −0.208 | 9.77E−01 | FALSE |
| Gmppa | 0.080 | 9.31E−01 | −0.438 | 9.96E−01 | 0.486 | 9.29E−02 | FALSE |
| Acat2 | −0.316 | 9.35E−02 | 0.199 | 1.00E+00 | 0.179 | 1.00E+00 | FALSE |
| Abtb1 | 0.305 | 9.43E−02 | −0.168 | 1.00E+00 | 0.284 | 7.36E−01 | FALSE |
| Cers4 | −0.066 | 1.00E+00 | −1.014 | 9.45E−02 | −0.577 | 1.21E−01 | FALSE |
| Ranbp1 | −0.239 | 9.48E−02 | 0.214 | 1.00E+00 | −0.069 | 1.00E+00 | FALSE |
| Rpl26 | 0.073 | 2.14E−01 | 0.036 | 1.00E+00 | 0.161 | 9.52E−02 | FALSE |
| Mif4gd | 0.264 | 9.63E−02 | 0.345 | 1.00E+00 | 0.042 | 1.00E+00 | FALSE |
| Hspa9 | −0.286 | 9.69E−02 | 0.368 | 1.00E+00 | −0.011 | 1.00E+00 | FALSE |
| Gpr15 | −0.155 | 9.74E−02 | −0.349 | 7.11E−01 | 0.053 | 1.00E+00 | FALSE |
| Sprtn | −0.143 | 9.77E−02 | 0.000 | 1.00E+00 | −0.093 | 1.00E+00 | FALSE |
| Usp16 | −0.308 | 9.85E−02 | −0.380 | 1.00E+00 | −0.080 | 1.00E+00 | FALSE |
| Ptpn6 | 0.294 | 9.87E−02 | 0.292 | 1.00E+00 | −0.008 | 1.00E+00 | FALSE |
| Gm26532 | −0.361 | 9.87E−02 | 0.102 | 1.00E+00 | −0.009 | 1.00E+00 | FALSE |
| Hba-a2 | 0.025 | 9.31E−01 | 0.062 | 1.00E+00 | 0.176 | 9.90E−02 | FALSE |
| Sit1 | −0.284 | 9.92E−02 | 0.223 | 1.00E+00 | −0.047 | 1.00E+00 | FALSE |
| Skil | −0.267 | 9.94E−02 | 0.407 | 1.00E+00 | −0.085 | 1.00E+00 | FALSE |
| Kat8 | 0.275 | 9.94E−02 | 0.712 | 4.66E−01 | −0.165 | 9.89E−01 | FALSE |
| Rhoh | −0.058 | 9.95E−01 | −0.435 | 9.96E−01 | −0.488 | 9.97E−02 | FALSE |

TABLE 4
Table detailing the ranking procedure used to prioritize genes for follow-up study. Gathers, for each
gene, the final score used for ranking in addition to the component statistics used in computing this
score. Details on the formula used are provided in the methods section. The scores above 2 are shown.

| | Final | In-Vivo | In-Vivo | In-Vitro | | | | |
|---|---|---|---|---|---|---|---|---|
| Gene | Score | Cluster | Tissue | Summary | GWAS? | LPL-9_logFC | LPL-9_FDR | LPL-9_Geno_logFC |
| Gpr18 | 6.04 | 0.87 | 0.96 | 1.34 | 1 | 0.05 | 9.16E−01 | −1.21 |
| Gzmb | 5.33 | 0.74 | 1.73 | 2.11 | 0 | −0.18 | 8.28E−01 | −0.31 |
| Itgb1 | 5.07 | 1.75 | 0.00 | 1.57 | 0 | −0.52 | 1.23E−01 | 0.62 |
| Ccr3 | 5.00 | 0.00 | 0.00 | 3.00 | 1 | 0.02 | 9.16E−01 | −0.06 |
| Gzma | 5.00 | 1.73 | 1.55 | 0.00 | 0 | 1.65 | 1.07E−14 | −2.92 |
| Il22 | 4.80 | 0.00 | 2.00 | 2.80 | 0 | −1.98 | 6.26E−05 | 0.12 |
| Zfp36l2 | 4.80 | 0.76 | 1.28 | 0.00 | 1 | −0.51 | 3.71E−03 | −1.28 |
| Zbtb38 | 4.72 | 0.30 | 0.21 | 1.90 | 1 | 0.61 | 1.07E−03 | −0.38 |
| Cd74 | 4.58 | 0.29 | 0.47 | 1.53 | 1 | −0.71 | 3.68E−02 | 0.39 |
| Ccr5 | 4.55 | 0.70 | 1.14 | 0.00 | 1 | −0.01 | 9.77E−01 | −0.29 |
| Cd160 | 4.15 | 1.39 | 1.38 | 0.00 | 0 | 2.21 | 8.92E−39 | −1.39 |
| Map3k8 | 4.11 | 0.27 | 1.57 | 0.00 | 1 | −1.07 | 1.04E−07 | −0.05 |
| Tnfsf8 | 4.00 | 0.00 | 2.00 | 0.00 | 1 | −2.58 | 3.47E−29 | 0.92 |
| Ifitm1 | 4.00 | 2.00 | 0.00 | 0.00 | 0 | 0.42 | 7.73E−01 | 0.06 |
| Nfkbiz | 4.00 | 0.00 | 2.00 | 0.00 | 1 | −1.84 | 5.04E−26 | 0.00 |
| Fosl2 | 4.00 | 0.00 | 2.00 | 0.00 | 1 | −2.22 | 2.11E−28 | −0.04 |
| Crem | 4.00 | 0.00 | 2.00 | 0.00 | 1 | −2.64 | 3.66E−42 | 0.29 |
| Ccdc85b | 4.00 | 0.24 | 0.31 | 1.20 | 1 | 0.49 | 5.14E−03 | 0.25 |
| Fos | 3.94 | 0.00 | 1.94 | 0.00 | 1 | −0.39 | 3.85E−01 | 0.88 |
| Gpr183 | 3.77 | 0.21 | 1.35 | 0.00 | 1 | −1.70 | 2.41E−24 | −0.09 |
| S100a4 | 3.76 | 1.53 | 0.71 | 0.00 | 0 | 0.21 | 5.67E−01 | −0.64 |
| 1110008f13rik | 3.63 | 0.32 | 0.00 | 3.00 | 0 | −0.24 | 3.26E−01 | −0.22 |
| Lsp1 | 3.63 | 0.61 | 0.41 | 0.00 | 1 | 0.54 | 2.06E−11 | 0.01 |
| Litaf | 3.61 | 0.00 | 1.61 | 0.00 | 1 | −0.08 | 9.16E−01 | −0.60 |
| Cd7 | 3.60 | 1.10 | 1.40 | 0.00 | 0 | 1.84 | 6.17E−25 | −1.93 |
| Dusp2 | 3.59 | 0.36 | 0.42 | 2.46 | 0 | 0.71 | 8.19E−05 | −1.01 |
| Plac8 | 3.59 | 1.00 | 1.58 | 0.00 | 0 | −0.08 | 9.16E−01 | −0.89 |
| H1f0 | 3.56 | 0.57 | 0.75 | 1.67 | | 0.24 | 5.20E−01 | 0.55 |
| S1pr1 | 3.56 | 0.88 | 0.42 | 1.37 | 0 | −0.16 | 8.29E−01 | −0.60 |
| Ncf4 | 3.53 | 0.62 | 0.29 | 0.00 | 1 | 0.30 | 2.08E−01 | 0.21 |
| Smim3 | 3.46 | 0.00 | 1.46 | 0.00 | 1 | −0.48 | 4.13E−02 | −0.11 |
| Tesc | 3.43 | 0.00 | 0.47 | 2.96 | 0 | 0.01 | 9.59E−01 | −0.05 |
| Rbms1 | 3.39 | 0.29 | 0.18 | 2.64 | 0 | 0.00 | 9.81E−01 | −0.27 |
| Lpxn | 3.39 | 0.21 | 0.97 | 0.00 | 1 | −0.02 | 9.16E−01 | −0.25 |
| Tnfrsf9 | 3.37 | 0.00 | 1.37 | 0.00 | 1 | −1.43 | 4.83E−10 | 0.51 |
| Pmm1 | 3.37 | 0.14 | 0.15 | 0.95 | 1 | 0.16 | 7.88E−01 | −0.41 |
| Tob2 | 3.32 | 0.00 | 1.32 | 0.00 | 1 | −1.09 | 4.21E−08 | 0.30 |
| Ifng | 3.32 | 0.00 | 1.69 | 1.63 | 0 | −1.20 | 7.19E−06 | 0.66 |
| Cd226 | 3.32 | 0.55 | 0.23 | 0.00 | 1 | 1.09 | 1.32E−13 | −0.19 |
| Ctsw | 3.28 | 0.47 | 0.35 | 0.00 | 1 | 0.93 | 3.13E−26 | −0.23 |
| Itga4 | 3.26 | 0.41 | 0.44 | 0.00 | 1 | 0.25 | 3.38E−01 | −0.60 |
| Cd28 | 3.23 | 0.08 | 1.08 | 0.00 | 1 | −1.13 | 4.90E−26 | −0.16 |
| Klf2 | 3.21 | 1.25 | 0.71 | 0.00 | 0 | −0.39 | 2.50E−01 | −1.19 |
| Ly6c2 | 3.19 | 1.60 | 0.00 | 0.00 | 0 | −0.73 | 1.12E−01 | −0.47 |
| Ccr9 | 3.19 | 1.11 | 0.97 | 0.00 | 0 | 1.55 | 7.75E−14 | −2.22 |
| Ets1 | 3.19 | 0.16 | 0.87 | 0.00 | 1 | −0.57 | 2.15E−09 | 0.03 |
| Il7r | 3.19 | 0.35 | 0.48 | 0.00 | 1 | 0.43 | 2.27E−03 | −0.54 |
| Ptpn22 | 3.18 | 0.00 | 1.18 | 0.00 | 1 | −0.69 | 1.93E−09 | −0.57 |
| Gm12840 | 3.17 | 0.59 | 2.00 | 0.00 | 0 | −1.43 | 4.99E−14 | −0.09 |
| Zfp36l1 | 3.17 | 0.00 | 1.17 | 0.00 | 1 | −0.78 | 3.31E−05 | −0.69 |
| Atxn2l | 3.17 | 0.00 | 0.07 | 1.10 | 1 | −0.12 | 8.69E−01 | −0.04 |
| Pde4b | 3.17 | 0.00 | 2.00 | 1.17 | 0 | −1.76 | 8.34E−28 | −0.20 |
| Selp | 3.16 | 0.00 | 0.00 | 1.16 | 1 | 0.00 | 9.16E−01 | 0.00 |
| Anxa6 | 3.16 | 0.48 | 0.20 | 0.00 | 1 | 0.30 | 2.42E−02 | 0.22 |
| Peli1 | 3.14 | 0.00 | 0.92 | 2.22 | 0 | −1.00 | 7.94E−13 | 0.23 |
| Odc1 | 3.12 | 0.00 | 2.00 | 1.12 | 0 | −2.92 | 6.12E−65 | 0.23 |
| Pglyrp1 | 3.12 | 1.21 | 0.70 | 0.00 | 0 | 0.12 | 8.90E−01 | −0.30 |
| Pim3 | 3.12 | 0.00 | 1.12 | 0.00 | 1 | −1.08 | 4.71E−07 | 0.03 |
| Pim1 | 3.11 | 0.56 | 2.00 | 0.00 | 0 | −1.85 | 2.32E−50 | −1.11 |
| Mt1 | 3.08 | 0.69 | 0.65 | 1.06 | 0 | −0.74 | 9.16E−03 | −0.32 |
| Stat3 | 3.08 | 0.00 | 1.08 | 0.00 | 1 | −0.85 | 1.15E−07 | 0.28 |
| Stat5a | 3.07 | 0.00 | 0.47 | 0.60 | 1 | −0.41 | 9.10E−02 | −0.12 |
| Vmp1 | 3.05 | 0.27 | 0.51 | 0.00 | 1 | −0.11 | 8.77E−01 | −0.82 |
| Ier2 | 3.05 | 0.21 | 0.43 | 2.19 | 0 | −0.98 | 4.39E−12 | −0.26 |
| Tnip1 | 3.02 | 0.13 | 0.77 | 0.00 | 1 | −0.88 | 1.43E−06 | 0.41 |
| Timp1 | 3.00 | 0.00 | 0.00 | 3.00 | 0 | 0.00 | 9.16E−01 | 0.00 |
| Amdhd1 | 3.00 | 0.00 | 0.00 | 3.00 | 0 | −0.03 | 7.89E−01 | 0.00 |
| Rora | 2.99 | 0.12 | 1.63 | 1.12 | 0 | −1.51 | 6.78E−27 | 0.19 |
| Gm5111 | 2.99 | 0.00 | 0.02 | 2.97 | 0 | 0.04 | 7.22E−01 | −0.06 |
| Fermt3 | 2.99 | 0.31 | 0.36 | 0.00 | 1 | 0.41 | 1.78E−02 | −0.04 |
| Dap | 2.96 | 0.34 | 0.28 | 0.00 | 1 | 0.06 | 9.16E−01 | −0.72 |
| Hist1h1c | 2.94 | 0.52 | 1.90 | 0.00 | 0 | −1.01 | 9.38E−06 | 0.17 |
| Cfl1 | 2.93 | 0.21 | 0.14 | 0.38 | 1 | 0.41 | 1.20E−13 | 0.33 |
| Ipmk | 2.92 | 0.00 | 0.92 | 0.00 | 1 | −0.84 | 4.49E−05 | −0.25 |
| Klrd1 | 2.90 | 1.28 | 0.34 | 0.00 | 0 | 0.21 | 6.79E−01 | 1.08 |
| Gfi1 | 2.89 | 0.40 | 0.09 | 0.00 | 1 | 0.81 | 5.66E−07 | −0.52 |
| S100a6 | 2.89 | 1.20 | 0.50 | 0.00 | 0 | 0.44 | 3.37E−04 | 0.25 |
| Rorc | 2.89 | 0.00 | 0.07 | 0.82 | 1 | 0.05 | 9.16E−01 | 0.05 |
| Stat4 | 2.87 | 0.00 | 0.87 | 0.00 | 1 | −0.54 | 2.67E−03 | 0.11 |
| Fdps | 2.86 | 0.36 | 0.15 | 0.00 | 1 | 0.71 | 7.75E−05 | −0.21 |
| Gpr65 | 2.86 | 0.00 | 0.86 | 0.00 | 1 | −0.53 | 2.06E−02 | −0.36 |
| Rapgef6 | 2.85 | 0.22 | 0.41 | 0.00 | 1 | 0.44 | 1.06E−02 | −0.40 |
| Tmem160 | 2.84 | 0.27 | 0.20 | 2.12 | 0 | 0.16 | 4.72E−01 | 0.20 |
| Rasgrp1 | 2.84 | 0.00 | 0.84 | 0.00 | 1 | −0.60 | 1.41E−03 | −0.39 |
| Pnkd | 2.83 | 0.31 | 0.21 | 0.00 | 1 | 0.63 | 1.17E−04 | −0.58 |
| Ccnd3 | 2.82 | 0.41 | 0.00 | 0.00 | 1 | 0.58 | 9.30E−06 | 0.30 |
| Panx1 | 2.82 | 0.20 | 0.00 | 2.42 | 0 | −0.11 | 9.10E−01 | 0.22 |
| Thap11 | 2.81 | 0.31 | 0.13 | 2.06 | 0 | 0.62 | 2.48E−04 | −0.56 |
| Lysmd2 | 2.80 | 0.34 | 0.25 | 1.86 | 0 | 0.26 | 4.08E−01 | −0.14 |
| Ubac2 | 2.80 | 0.25 | 0.29 | 0.00 | 1 | 0.51 | 7.50E−04 | −0.24 |
| Lgals3 | 2.79 | 1.40 | 0.00 | 0.00 | 0 | −0.27 | 2.98E−01 | 0.46 |
| Cyb5r4 | 2.78 | 0.32 | 0.29 | 1.86 | 0 | 0.22 | 4.11E−01 | 0.13 |
| Upf3a | 2.78 | 0.39 | 0.34 | 1.66 | 0 | 0.22 | 5.09E−01 | 0.13 |
| Gm10076 | 2.78 | 1.04 | 0.69 | 0.00 | 0 | 0.07 | 9.16E−01 | −0.36 |
| Ccdc69 | 2.77 | 0.30 | 0.18 | 0.00 | 1 | 0.59 | 2.73E−04 | −0.40 |
| Ccl5 | 2.76 | 1.13 | 0.50 | 0.00 | 0 | 0.92 | 7.28E−10 | −1.43 |
| Gramd3 | 2.75 | 0.42 | 1.92 | 0.00 | 0 | −2.80 | 1.99E−64 | −0.50 |
| Errfi1 | 2.75 | 0.00 | 0.75 | 0.00 | 1 | −0.85 | 6.10E−05 | 0.00 |
| Itgae | 2.75 | 0.86 | 1.03 | 0.00 | 0 | 1.72 | 6.36E−26 | −1.44 |
| Il2ra | 2.74 | 0.00 | 0.74 | 0.00 | 1 | −0.71 | 7.84E−04 | −0.03 |
| Gpx4 | 2.72 | 0.00 | 0.72 | 0.00 | 1 | −0.61 | 3.04E−08 | 0.17 |
| Ndfip1 | 2.72 | 0.00 | 0.72 | 0.00 | 1 | −1.00 | 1.29E−12 | −0.02 |
| Syt11 | 2.72 | 0.31 | 0.11 | 0.00 | 1 | 0.61 | 6.33E−04 | −0.13 |
| Dph5 | 2.71 | 0.00 | 0.00 | 0.71 | 1 | −0.34 | 1.73E−01 | 0.43 |
| Slc39a8 | 2.70 | 0.00 | 0.08 | 0.62 | 1 | 0.25 | 2.07E−01 | 0.49 |
| Rnf187 | 2.69 | 0.00 | 0.00 | 2.69 | 0 | −0.03 | 9.16E−01 | −0.04 |
| Fam53b | 2.68 | 0.27 | 0.14 | 0.00 | 1 | 0.54 | 4.97E−03 | −0.40 |
| Prdx5 | 2.68 | 0.15 | 0.37 | 0.00 | 1 | 0.30 | 3.27E−02 | 0.36 |
| Ccrl2 | 2.67 | 0.00 | 0.67 | 0.00 | 1 | −0.50 | 4.03E−02 | 0.57 |
| Ep300 | 2.67 | 0.00 | 0.00 | 0.67 | 1 | −0.02 | 9.22E−01 | −0.09 |
| Hdac7 | 2.66 | 0.28 | 0.10 | 0.00 | 1 | 0.19 | 5.54E−01 | −0.17 |
| Cd6 | 2.66 | 0.23 | 0.21 | 0.00 | 1 | 0.16 | 4.22E−01 | 0.22 |
| Traf3ip2 | 2.66 | 0.29 | 0.09 | 0.00 | 1 | −0.01 | 9.79E−01 | −0.46 |
| Gnb2 | 2.66 | 0.20 | 0.25 | 0.00 | 1 | 0.14 | 4.84E−01 | 0.28 |
| Tet2 | 2.66 | 0.00 | 0.66 | 0.00 | 1 | −0.33 | 2.15E−01 | 0.29 |
| Fam189b | 2.66 | 0.33 | 0.00 | 0.00 | 1 | 0.66 | 1.50E−05 | −0.30 |
| Skap2 | 2.65 | 0.26 | 0.13 | 0.00 | 1 | 0.53 | 4.39E−03 | 0.03 |
| Lgals1 | 2.64 | 1.11 | 0.17 | 0.25 | 0 | 0.03 | 9.16E−01 | 0.37 |
| Socs1 | 2.64 | 0.00 | 0.64 | 0.00 | 1 | −1.20 | 3.60E−09 | 0.36 |
| Itgav | 2.64 | 0.00 | 0.64 | 0.00 | 1 | −0.46 | 5.45E−02 | −0.44 |
| Dnajb4 | 2.63 | 0.00 | 0.63 | 0.00 | 1 | −0.35 | 2.20E−01 | 0.13 |
| Cdkn1a | 2.63 | 0.00 | 2.00 | 0.63 | 0 | −1.97 | 4.97E−22 | 0.16 |
| Arhgef2 | 2.62 | 0.20 | 0.23 | 0.00 | 1 | 0.40 | 5.38E−02 | 0.01 |
| Dnmt3a | 2.62 | 0.26 | 0.11 | 0.00 | 1 | 0.51 | 2.67E−03 | −0.34 |
| Jund | 2.61 | 0.00 | 2.00 | 0.61 | 0 | −1.56 | 1.41E−54 | 0.96 |
| Scamp3 | 2.61 | 0.19 | 0.23 | 0.00 | 1 | 0.38 | 2.05E−02 | −0.28 |
| Tmem30a | 2.61 | 0.35 | 0.35 | 1.55 | 0 | 0.22 | 3.63E−01 | −0.73 |
| Ash1l | 2.60 | 0.30 | 0.00 | 0.00 | 1 | 0.60 | 6.06E−04 | −0.13 |
| Lsm14a | 2.60 | 0.17 | 0.26 | 0.00 | 1 | 0.34 | 7.25E−02 | −0.04 |
| Slain2 | 2.60 | 0.00 | 0.00 | 0.60 | 1 | −0.16 | 7.57E−01 | 0.25 |
| Muc1 | 2.60 | 0.11 | 0.38 | 0.00 | 1 | −0.31 | 1.19E−01 | −0.12 |
| Adamts14 | 2.59 | 0.00 | 0.00 | 2.59 | 0 | 0.00 | 9.16E−01 | 0.00 |
| Ptk2b | 2.59 | 0.25 | 0.09 | 0.00 | 1 | 0.50 | 3.11E−03 | 0.23 |
| Stk11 | 2.59 | 0.00 | 0.59 | 0.00 | 1 | −0.38 | 7.50E−02 | −0.01 |
| Ccr2 | 2.58 | 0.17 | 0.23 | 0.00 | 1 | −0.82 | 5.53E−07 | 0.19 |
| Itgal | 2.57 | 0.23 | 0.11 | 0.00 | 1 | 0.47 | 4.68E−03 | −0.85 |
| Elf1 | 2.57 | 0.00 | 0.57 | 0.00 | 1 | −0.12 | 8.01E−01 | −0.44 |
| Ptprc | 2.57 | 0.20 | 0.16 | 0.00 | 1 | 0.05 | 9.16E−01 | −0.26 |
| Rbx1 | 2.56 | 0.21 | 0.14 | 0.00 | 1 | 0.42 | 4.59E−05 | 0.23 |
| Traf3ip3 | 2.56 | 0.44 | 0.45 | 1.23 | 0 | 0.79 | 1.50E−07 | −0.46 |
| Ccdc88b | 2.55 | 0.24 | 0.08 | 0.00 | 1 | 0.48 | 1.39E−02 | −0.36 |
| Tsen34 | 2.55 | 0.23 | 0.00 | 2.09 | 0 | 0.46 | 2.92E−03 | −0.30 |
| Ay036118 | 2.54 | 1.15 | 0.25 | 0.00 | 0 | −0.70 | 9.08E−04 | 0.26 |
| Il24 | 2.54 | 0.00 | 0.00 | 2.54 | 0 | | | |
| H2-q10 | 2.54 | 0.00 | 0.06 | 2.48 | 0 | −0.40 | 9.22E−02 | −0.05 |
| Scand1 | 2.54 | 0.16 | 0.38 | 1.83 | 0 | 0.11 | 7.40E−01 | −0.08 |
| Tab2 | 2.54 | 0.14 | 0.26 | 0.00 | 1 | 0.14 | 7.87E−01 | 0.31 |
| Nfkb1 | 2.53 | 0.00 | 0.53 | 0.00 | 1 | −0.80 | 1.07E−06 | 0.41 |
| Rusc1 | 2.51 | 0.20 | 0.11 | 0.00 | 1 | 0.40 | 5.20E−02 | −0.38 |
| Esyt1 | 2.51 | 0.48 | 0.24 | 1.32 | 0 | 0.50 | 2.99E−03 | 0.14 |
| Commd7 | 2.51 | 0.21 | 0.08 | 0.00 | 1 | 0.42 | 1.69E−02 | 0.43 |
| Rfng | 2.50 | 0.21 | 0.14 | 1.94 | 0 | 0.42 | 1.91E−02 | −0.41 |
| Rpl27-ps3 | 2.50 | 1.05 | 0.40 | 0.00 | 0 | −0.39 | 1.47E−02 | −0.81 |
| Gigyf1 | 2.50 | 0.25 | 0.00 | 0.00 | 1 | 0.50 | 3.73E−03 | 0.04 |
| Rgcc | 2.49 | 0.00 | 1.58 | 0.92 | 0 | −2.01 | 3.98E−17 | 0.81 |
| Rpl3 | 2.49 | 0.15 | 0.19 | 0.00 | 1 | 0.03 | 8.98E−01 | −0.17 |
| Mrpl20 | 2.49 | 0.16 | 0.16 | 0.00 | 1 | −0.12 | 8.00E−01 | −0.15 |
| Gm2a | 2.48 | 0.15 | 0.18 | 0.00 | 1 | 0.30 | 9.19E−02 | 0.11 |
| Mus81 | 2.47 | 0.17 | 0.13 | 0.00 | 1 | 0.34 | 6.54E−02 | −0.25 |
| Lpp | 2.47 | 0.00 | 0.00 | 0.47 | 1 | 0.02 | 9.32E−01 | 0.11 |
| Chp1 | 2.47 | 0.16 | 0.14 | 0.00 | 1 | 0.24 | 3.53E−01 | −0.58 |
| Mob4 | 2.46 | 0.11 | 0.24 | 0.00 | 1 | −0.16 | 6.52E−01 | −0.20 |
| Lamp1 | 2.46 | 0.00 | 0.08 | 2.38 | 0 | 0.17 | 5.79E−01 | −0.15 |
| Phf5a | 2.46 | 0.16 | 0.14 | 0.00 | 1 | −0.18 | 5.90E−01 | −0.06 |
| Fnip1 | 2.46 | 0.11 | 0.25 | 0.00 | 1 | −0.35 | 5.02E−02 | 0.12 |
| Calm3 | 2.45 | 0.19 | 0.07 | 0.00 | 1 | 0.38 | 3.36E−03 | 0.27 |
| Lrp10 | 2.45 | 0.21 | 0.20 | 1.82 | 0 | 0.43 | 9.66E−03 | 0.00 |
| Stat5b | 2.45 | 0.00 | 0.45 | 0.00 | 1 | −0.42 | 7.05E−02 | −0.17 |
| Bach2 | 2.45 | 0.00 | 0.00 | 0.45 | 1 | −0.19 | 9.99E−02 | 0.00 |
| Prkab1 | 2.44 | 0.22 | 0.00 | 0.00 | 1 | 0.44 | 1.85E−02 | −0.07 |
| Hvcn1 | 2.44 | 0.39 | 0.00 | 1.65 | 0 | 0.79 | 3.23E−07 | −0.51 |
| Tmem150a | 2.44 | 0.00 | 0.00 | 2.44 | 0 | −0.01 | 9.16E−01 | 0.00 |
| Nfkbia | 2.44 | 0.22 | 2.00 | 0.00 | 0 | −2.00 | 6.41E−52 | 0.23 |
| Ifitm3 | 2.43 | 1.07 | 0.28 | 0.00 | 0 | −0.15 | 8.69E−01 | −0.26 |
| Egfp_ires_il23r | 2.43 | 0.00 | 0.00 | 2.43 | 0 | | | |
| Bad | 2.43 | 0.15 | 0.13 | 0.00 | 1 | 0.25 | 3.65E−01 | −0.14 |
| Zbtb46 | 2.42 | 0.10 | 0.21 | 0.00 | 1 | −0.24 | 4.01E−02 | 0.00 |
| Cacna1s | 2.42 | 0.00 | 0.10 | 0.32 | 1 | −0.02 | 9.16E−01 | 0.12 |
| Vamp3 | 2.42 | 0.15 | 0.11 | 0.00 | 1 | −0.12 | 8.75E−01 | −0.20 |
| Fyn | 2.41 | 0.21 | 0.00 | 0.00 | 1 | 0.41 | 6.91E−03 | 0.01 |
| Stx6 | 2.41 | 0.00 | 0.29 | 2.12 | 0 | −0.54 | 9.36E−03 | 0.33 |
| Ikzf3 | 2.41 | 0.00 | 0.41 | 0.00 | 1 | 0.03 | 9.16E−01 | 0.01 |
| Mgat2 | 2.41 | 0.17 | 0.00 | 2.07 | 0 | −0.13 | 7.90E−01 | 0.01 |
| Serpinb9 | 2.41 | 0.35 | 1.70 | 0.00 | 0 | −2.27 | 2.37E−29 | 0.48 |
| Dennd1b | 2.41 | 0.20 | 0.00 | 0.00 | 1 | 0.41 | 4.95E−02 | 0.56 |
| Bloc1s4 | 2.40 | 0.00 | 0.27 | 2.13 | 0 | −0.32 | 2.26E−01 | 0.29 |
| Anxa1 | 2.40 | 1.20 | 0.00 | 0.00 | 0 | −0.39 | 2.24E−01 | 0.51 |
| Smad3 | 2.39 | 0.15 | 0.10 | 0.00 | 1 | −0.17 | 6.80E−01 | −0.20 |
| Tmem258 | 2.39 | 0.16 | 0.08 | 0.00 | 1 | 0.31 | 1.20E−02 | −0.15 |
| Lmna | 2.39 | 0.20 | 1.99 | 0.00 | 0 | −2.18 | 3.54E−21 | 1.52 |
| Sp140 | 2.38 | 0.15 | 0.09 | 0.00 | 1 | 0.07 | 9.16E−01 | −0.07 |
| Actn2 | 2.38 | 0.56 | 1.25 | 0.00 | 0 | 0.97 | 1.90E−11 | −0.17 |
| Acbd6 | 2.38 | 0.00 | 0.22 | 2.16 | 0 | −0.20 | 5.59E−01 | −0.03 |
| Ahnak | 2.38 | 0.50 | 0.16 | 1.22 | 0 | −0.68 | 6.25E−06 | −0.15 |
| Arpc2 | 2.37 | 0.16 | 0.06 | 0.00 | 1 | −0.21 | 6.08E−03 | 0.10 |
| Erp29 | 2.37 | 0.19 | 0.00 | 0.00 | 1 | 0.09 | 9.16E−01 | −0.38 |
| Nhp2l1 | 2.37 | 0.00 | 0.37 | 0.00 | 1 | −0.78 | 2.02E−09 | 0.23 |
| Ctbp1 | 2.36 | 0.35 | 0.32 | 1.34 | 0 | −0.18 | 5.38E−01 | −0.18 |
| Gba | 2.36 | 0.11 | 0.13 | 0.00 | 1 | 0.19 | 5.83E−01 | −0.11 |
| Lztfl1 | 2.35 | 0.56 | 1.24 | 0.00 | 0 | 0.50 | 7.25E−03 | −0.48 |
| Ptpn2 | 2.35 | 0.00 | 0.35 | 0.00 | 1 | −0.50 | 1.46E−02 | 0.14 |
| Usp36 | 2.35 | 0.00 | 0.35 | 0.00 | 1 | −0.33 | 2.19E−01 | 0.35 |
| Grb7 | 2.34 | 0.00 | 0.34 | 0.00 | 1 | −0.23 | 3.56E−01 | 0.28 |
| Fndc3a | 2.34 | 0.00 | 0.34 | 0.00 | 1 | −0.27 | 3.66E−01 | 0.38 |
| Mt2 | 2.33 | 0.41 | 0.21 | 1.32 | 0 | −0.12 | 9.16E−01 | −0.21 |
| Dctn4 | 2.33 | 0.00 | 0.33 | 0.00 | 1 | −0.13 | 8.69E−01 | −0.49 |
| Nusap1 | 2.33 | 0.00 | 0.33 | 0.00 | 1 | −0.47 | 2.85E−02 | −0.06 |
| Rbm22 | 2.33 | 0.16 | 0.00 | 0.00 | 1 | 0.31 | 1.24E−01 | −0.08 |
| Crip1 | 2.32 | 1.16 | 0.00 | 0.00 | 0 | −0.16 | 4.44E−01 | 0.13 |
| Anp32b | 2.32 | 0.00 | 0.19 | 2.13 | 0 | −0.03 | 9.16E−01 | 0.09 |
| Aurkaip1 | 2.31 | 0.16 | 0.00 | 0.00 | 1 | 0.31 | 4.66E−02 | 0.23 |
| Ripk2 | 2.31 | 0.21 | 0.36 | 1.53 | 0 | −0.31 | 2.52E−01 | −0.29 |
| Nubp1 | 2.31 | 0.12 | 0.37 | 1.70 | 0 | 0.05 | 9.16E−01 | −0.11 |
| Camta1 | 2.31 | 0.00 | 0.31 | 0.00 | 1 | 0.15 | 7.66E−01 | 0.04 |
| Ube2j2 | 2.30 | 0.00 | 0.30 | 0.00 | 1 | −0.51 | 1.26E−03 | −0.09 |
| Kif3b | 2.30 | 0.00 | 0.00 | 0.30 | 1 | 0.21 | 2.31E−01 | 0.43 |
| Brap | 2.30 | 0.00 | 0.30 | 0.00 | 1 | −0.36 | 1.42E−01 | −0.02 |
| Gm8096 | 2.30 | 0.00 | 0.00 | 2.30 | 0 | | | |
| Ermn | 2.30 | 0.00 | 1.01 | 1.29 | 0 | −0.82 | 2.08E−03 | 0.18 |
| Plagl2 | 2.29 | 0.00 | 0.00 | 0.29 | 1 | 0.11 | 8.12E−01 | −0.24 |
| Plek | 2.29 | 0.44 | 0.00 | 1.42 | 0 | −0.17 | 7.62E−01 | −0.02 |
| D030056l22rik | 2.29 | 0.00 | 0.23 | 2.06 | 0 | −0.22 | 5.63E−01 | 0.12 |
| Ccr1 | 2.28 | 0.00 | 0.28 | 0.00 | 1 | −0.25 | 3.19E−01 | 0.33 |
| Krtcap2 | 2.28 | 0.14 | 0.00 | 0.00 | 1 | 0.15 | 2.54E−01 | 0.68 |
| Sh2b1 | 2.28 | 0.00 | 0.28 | 0.00 | 1 | 0.00 | 9.89E−01 | 0.10 |
| Abi3 | 2.27 | 0.65 | 0.97 | 0.00 | 0 | 1.31 | 2.58E−20 | −0.41 |
| Ccnl2 | 2.27 | 0.00 | 0.27 | 0.00 | 1 | −0.11 | 8.44E−01 | −0.32 |
| Kbtbd7 | 2.27 | 0.14 | 0.00 | 0.00 | 1 | 0.27 | 3.02E−02 | 0.32 |
| Csf2 | 2.27 | 0.00 | 0.27 | 0.00 | 1 | −0.27 | 4.91E−01 | 0.12 |
| Pycard | 2.27 | 0.82 | 0.63 | 0.00 | 0 | 0.67 | 2.06E−07 | −0.41 |
| Dock7 | 2.27 | 0.00 | 0.00 | 0.27 | 1 | 0.00 | 9.16E−01 | 0.00 |
| Herpud1 | 2.26 | 0.00 | 1.16 | 1.10 | 0 | −1.41 | 4.94E−17 | −0.99 |
| Arf6 | 2.26 | 0.12 | 0.39 | 1.63 | 0 | −0.07 | 8.95E−01 | −0.09 |
| Zyx | 2.26 | 0.87 | 0.52 | 0.00 | 0 | 0.34 | 1.46E−01 | −0.27 |
| Zfp91 | 2.25 | 0.00 | 0.25 | 0.00 | 1 | −0.19 | 6.33E−01 | 0.28 |
| Fam171b | 2.25 | 0.13 | 0.00 | 0.00 | 1 | 0.25 | 6.60E−03 | −0.05 |
| Maml2 | 2.25 | 0.12 | 0.00 | 0.00 | 1 | 0.28 | 3.01E−01 | 0.08 |
| Ifngr2 | 2.24 | 0.00 | 0.24 | 0.00 | 1 | −0.35 | 7.19E−03 | 0.00 |
| Bhlhe40 | 2.24 | 0.12 | 2.00 | 0.00 | 0 | −2.05 | 5.44E−83 | −0.58 |
| Cisd1 | 2.23 | 0.00 | 0.23 | 0.00 | 1 | 0.29 | 2.94E−01 | 0.39 |
| Zfp420 | 2.22 | 0.00 | 0.00 | 2.22 | 0 | 0.05 | 9.16E−01 | 0.32 |
| Zc3h12a | 2.22 | 0.11 | 2.00 | 0.00 | 0 | −1.90 | 3.03E−26 | 0.20 |
| Plcg2 | 2.22 | 0.08 | 0.05 | 0.00 | 1 | 0.14 | 3.14E−01 | −0.27 |
| 2310036o22rik | 2.21 | 0.00 | 0.13 | 2.09 | 0 | 0.10 | 8.00E−01 | 0.13 |
| Eif3c | 2.21 | 0.11 | 0.00 | 0.00 | 1 | −0.03 | 9.16E−01 | 0.00 |
| Slc15a3 | 2.21 | 0.00 | 0.52 | 1.69 | 0 | −0.62 | 2.28E−03 | −0.01 |
| Acap3 | 2.21 | 0.10 | 0.00 | 0.00 | 1 | 0.14 | 7.00E−01 | 0.20 |
| Tef | 2.21 | 0.00 | 0.21 | 0.00 | 1 | −0.20 | 4.12E−01 | 0.03 |
| Emp3 | 2.20 | 0.93 | 0.34 | 0.00 | 0 | 0.17 | 4.01E−01 | −0.30 |
| Ssr2 | 2.20 | 0.00 | 0.20 | 0.00 | 1 | −0.02 | 9.16E−01 | −0.24 |
| Usp1 | 2.19 | 0.00 | 0.19 | 0.00 | 1 | 0.05 | 9.16E−01 | 0.13 |
| Rpl10-ps3 | 2.18 | 0.89 | 0.40 | 0.00 | 0 | −0.20 | 4.07E−01 | −0.22 |
| Plcxd2 | 2.18 | 0.42 | 1.34 | 0.00 | 0 | −0.89 | 1.41E−05 | −0.23 |
| Adcy7 | 2.18 | 0.00 | 0.18 | 0.00 | 1 | 0.18 | 6.40E−01 | −0.04 |
| Irf1 | 2.18 | 0.00 | 0.18 | 0.00 | 1 | 0.00 | 9.81E−01 | −0.14 |
| Aamp | 2.17 | 0.00 | 0.17 | 0.00 | 1 | −0.08 | 9.16E−01 | 0.39 |
| Gm8730 | 2.17 | 0.78 | 0.61 | 0.00 | 0 | 0.03 | 9.16E−01 | −0.97 |
| Zgpat | 2.17 | 0.00 | 0.17 | 0.00 | 1 | 0.23 | 4.47E−01 | −0.28 |
| St13 | 2.17 | 0.00 | 0.17 | 0.00 | 1 | 0.03 | 9.16E−01 | −0.54 |
| Cdc42se2 | 2.17 | 0.00 | 0.17 | 0.00 | 1 | −0.02 | 9.36E−01 | −0.44 |
| Anxa2 | 2.16 | 0.69 | 0.00 | 0.77 | 0 | −0.14 | 6.71E−01 | 0.46 |
| Cpsf3l | 2.16 | 0.00 | 0.16 | 0.00 | 1 | 0.30 | 2.53E−01 | 0.25 |
| Gm2000 | 2.15 | 0.84 | 0.47 | 0.00 | 0 | −0.16 | 5.62E−01 | −0.56 |
| Trim46 | 2.15 | 0.08 | 0.00 | 0.00 | 1 | 0.00 | 9.76E−01 | −0.12 |
| Dusp16 | 2.15 | 0.00 | 0.15 | 0.00 | 1 | −0.16 | 1.48E−01 | 0.00 |
| Zmat5 | 2.15 | 0.00 | 0.15 | 0.00 | 1 | 0.30 | 1.96E−01 | 0.39 |
| Ptprk | 2.15 | 0.00 | 0.15 | 0.00 | 1 | 0.17 | 6.28E−01 | −0.02 |
| Zdhhc7 | 2.15 | 0.15 | 0.00 | 1.85 | 0 | 0.14 | 7.96E−01 | 0.64 |
| Il2 | 2.14 | 0.00 | 0.14 | 0.00 | 1 | 0.21 | 6.26E−01 | 0.62 |
| Msto1 | 2.14 | 0.00 | 0.14 | 0.00 | 1 | 0.22 | 3.66E−01 | 0.22 |
| Il10 | 2.14 | 0.00 | 0.14 | 0.00 | 1 | −0.26 | 1.52E−01 | 0.00 |
| Sbno2 | 2.14 | 0.00 | 0.14 | 0.00 | 1 | −0.14 | 4.66E−01 | −0.06 |
| Snx17 | 2.14 | 0.00 | 0.14 | 0.00 | 1 | 0.15 | 7.29E−01 | 0.11 |
| Kif11 | 2.14 | 0.00 | 0.14 | 0.00 | 1 | −0.14 | 3.28E−01 | −0.06 |
| Ndfip2 | 2.13 | 0.00 | 0.08 | 2.05 | 0 | −0.02 | 9.43E−01 | −0.19 |
| Card9 | 2.13 | 0.07 | 0.00 | 0.00 | 1 | 0.13 | 1.78E−02 | 0.26 |
| Lsm4 | 2.13 | 0.15 | 0.25 | 1.59 | 0 | 0.29 | 4.77E−02 | 0.35 |
| Ormdl3 | 2.13 | 0.00 | 0.13 | 0.00 | 1 | −0.22 | 5.67E−01 | −0.44 |
| S100a10 | 2.13 | 0.71 | 0.00 | 0.70 | 0 | 0.24 | 4.32E−03 | 0.34 |
| Notch2 | 2.13 | 0.00 | 0.13 | 0.00 | 1 | 0.28 | 3.07E−01 | −0.29 |
| Senp7 | 2.12 | 0.00 | 0.12 | 0.00 | 1 | 0.27 | 2.72E−01 | −0.03 |
| Cd48 | 2.12 | 0.80 | 0.52 | 0.00 | 0 | 1.09 | 4.17E−26 | −0.11 |
| Ssbp4 | 2.12 | 0.13 | 0.26 | 1.61 | 0 | 0.12 | 7.93E−01 | 0.07 |
| Cul1 | 2.12 | 0.00 | 0.12 | 0.00 | 1 | −0.24 | 4.52E−01 | 0.14 |
| Mettl10 | 2.12 | 0.00 | 0.12 | 0.00 | 1 | 0.32 | 1.67E−01 | 0.29 |
| Rabep2 | 2.11 | 0.00 | 0.11 | 0.00 | 1 | 0.25 | 3.59E−01 | 0.27 |
| Pkig | 2.11 | 0.00 | 0.11 | 0.00 | 1 | −0.11 | 7.47E−01 | 0.45 |
| Desi1 | 2.11 | 0.00 | 0.11 | 0.00 | 1 | −0.05 | 9.16E−01 | −0.44 |
| Furin | 2.11 | 0.00 | 2.00 | 0.11 | 0 | −1.32 | 2.08E−16 | 0.82 |
| Tcof1 | 2.11 | 0.00 | 0.11 | 0.00 | 1 | 0.16 | 7.20E−01 | −0.17 |
| Glipr2 | 2.11 | 0.84 | 0.43 | 0.00 | 0 | 0.28 | 1.81E−01 | −0.10 |
| Tgfbr3 | 2.11 | 0.00 | 0.11 | 0.00 | 1 | −0.12 | 6.95E−01 | −0.12 |
| Tyk2 | 2.10 | 0.00 | 0.10 | 0.00 | 1 | 0.24 | 2.94E−01 | −0.66 |
| Kif21b | 2.10 | 0.00 | 0.10 | 0.00 | 1 | 0.32 | 1.73E−01 | 0.12 |
| Tufm | 2.10 | 0.00 | 0.10 | 0.00 | 1 | 0.25 | 3.89E−01 | −0.14 |
| Zmiz1 | 2.10 | 0.00 | 0.10 | 0.00 | 1 | 0.04 | 9.16E−01 | −0.32 |
| Il21 | 2.10 | 0.00 | 0.10 | 0.00 | 1 | −0.08 | 8.15E−01 | −0.06 |
| Ttpal | 2.10 | 0.00 | 0.10 | 0.00 | 1 | 0.05 | 9.16E−01 | −0.33 |
| Rps6kb1 | 2.10 | 0.00 | 0.10 | 0.00 | 1 | −0.15 | 7.63E−01 | −0.71 |
| Camk2g | 2.10 | 0.00 | 0.10 | 0.00 | 1 | 0.07 | 9.16E−01 | 0.16 |
| Tom1 | 2.10 | 0.00 | 0.10 | 0.00 | 1 | 0.20 | 4.19E−01 | −0.14 |
| Actr1a | 2.10 | 0.00 | 0.10 | 0.00 | 1 | 0.15 | 7.74E−01 | −0.30 |
| Rgs14 | 2.10 | 0.00 | 0.10 | 0.00 | 1 | −0.03 | 9.16E−01 | −0.10 |
| Ublcp1 | 2.10 | 0.00 | 0.10 | 0.00 | 1 | −0.04 | 9.16E−01 | 0.10 |
| Otud3 | 2.09 | 0.00 | 0.09 | 0.00 | 1 | 0.01 | 9.56E−01 | 0.22 |
| Tmem180 | 2.09 | 0.00 | 0.09 | 0.00 | 1 | −0.03 | 9.16E−01 | −0.27 |
| Ergic1 | 2.09 | 0.00 | 0.09 | 0.00 | 1 | 0.02 | 9.53E−01 | −0.05 |
| Aa467197 | 2.09 | 0.97 | 0.16 | 0.00 | 0 | 0.42 | 4.68E−02 | −0.01 |
| Tdp2 | 2.09 | 0.00 | 0.25 | 1.84 | 0 | −0.56 | 1.00E−02 | 0.04 |
| Rac1 | 2.09 | 0.07 | 0.06 | 1.88 | 0 | −0.22 | 1.23E−01 | 0.22 |
| Phlda3 | 2.09 | 0.00 | 0.00 | 2.09 | 0 | −0.08 | 9.16E−01 | 0.12 |
| Trmt112 | 2.09 | 0.00 | 0.09 | 0.00 | 1 | 0.13 | 6.94E−01 | −0.19 |
| Ccny | 2.08 | 0.00 | 0.08 | 0.00 | 1 | −0.18 | 6.57E−01 | −0.22 |
| Slc43a3 | 2.08 | 0.00 | 0.08 | 0.00 | 1 | 0.03 | 9.16E−01 | −0.32 |
| Ezh2 | 2.08 | 0.00 | 0.08 | 0.00 | 1 | −0.30 | 2.75E−01 | −0.19 |
| Rps14 | 2.08 | 0.00 | 0.08 | 0.00 | 1 | −0.06 | 2.70E−01 | 0.00 |
| Spred2 | 2.08 | 0.00 | 0.08 | 0.00 | 1 | −0.13 | 6.12E−01 | 0.12 |
| Rn7s2 | 2.08 | 0.00 | 0.00 | 2.08 | 0 | | | |
| Rn7s1 | 2.08 | 0.00 | 0.00 | 2.08 | 0 | | | |
| Rpap2 | 2.08 | 0.00 | 0.08 | 0.00 | 1 | 0.19 | 4.73E−01 | 0.10 |
| Cit | 2.08 | 0.00 | 0.08 | 0.00 | 1 | −0.08 | 8.92E−01 | −0.16 |
| Ascc2 | 2.08 | 0.00 | 0.08 | 0.00 | 1 | 0.01 | 9.56E−01 | −0.15 |
| Ksr1 | 2.08 | 0.00 | 0.08 | 0.00 | 1 | −0.02 | 9.16E−01 | −0.18 |
| Edem2 | 2.08 | 0.00 | 0.08 | 0.00 | 1 | 0.35 | 1.30E−01 | −0.12 |
| Blzf1 | 2.08 | 0.00 | 0.08 | 0.00 | 1 | −0.04 | 9.16E−01 | 0.00 |
| Ppp5c | 2.08 | 0.00 | 0.08 | 0.00 | 1 | 0.22 | 4.78E−01 | −0.09 |
| Smurf1 | 2.08 | 0.00 | 0.08 | 0.00 | 1 | −0.17 | 3.56E−01 | 0.18 |
| Nop9 | 2.08 | 0.00 | 0.08 | 2.00 | 0 | −0.23 | 4.77E−01 | 0.05 |
| Slc30a7 | 2.08 | 0.00 | 0.08 | 0.00 | 1 | 0.07 | 9.16E−01 | −0.29 |
| Tnfrsf8 | 2.08 | 0.00 | 0.00 | 2.08 | 0 | −0.04 | 6.49E−01 | 0.00 |
| Adcy3 | 2.08 | 0.00 | 0.08 | 0.00 | 1 | 0.14 | 6.08E−01 | −0.10 |
| Pdcd1 | 2.07 | 0.00 | 0.00 | 2.07 | 0 | −0.01 | 9.93E−01 | 0.18 |
| Fen1 | 2.07 | 0.00 | 0.07 | 0.00 | 1 | −0.19 | 5.90E−01 | −0.08 |
| Themis | 2.07 | 0.00 | 0.07 | 0.00 | 1 | 0.21 | 4.38E−01 | −0.47 |
| Nudt22 | 2.07 | 0.00 | 0.07 | 0.00 | 1 | 0.28 | 2.11E−01 | −0.10 |
| Cyth1 | 2.07 | 0.00 | 0.07 | 0.00 | 1 | −0.12 | 8.69E−01 | 0.11 |
| Tmbim1 | 2.07 | 0.00 | 0.07 | 0.00 | 1 | 0.00 | 9.82E−01 | 0.35 |
| Ndufaf1 | 2.07 | 0.00 | 0.07 | 0.00 | 1 | 0.24 | 3.44E−01 | 0.13 |
| Klrc1 | 2.07 | 0.87 | 0.33 | 0.00 | 0 | −1.21 | 7.84E−09 | 0.71 |
| Pomc | 2.07 | 0.00 | 0.07 | 0.00 | 1 | −0.07 | 8.21E−01 | 0.17 |
| Cpeb4 | 2.07 | 0.00 | 0.07 | 0.00 | 1 | 0.06 | 9.16E−01 | 0.41 |
| Cul2 | 2.07 | 0.00 | 0.07 | 0.00 | 1 | −0.03 | 9.16E−01 | 0.19 |
| Hgfac | 2.07 | 0.03 | 0.00 | 0.00 | 1 | −0.02 | 9.09E−01 | 0.00 |
| Pofut1 | 2.07 | 0.00 | 0.07 | 0.00 | 1 | 0.16 | 5.63E−01 | −0.20 |
| Clasp2 | 2.07 | 0.00 | 0.06 | 2.00 | 0 | 0.08 | 9.16E−01 | −0.05 |
| Nme7 | 2.06 | 0.00 | 0.06 | 0.00 | 1 | 0.16 | 4.96E−01 | 0.66 |
| Ndst1 | 2.06 | 0.00 | 0.06 | 0.00 | 1 | −0.11 | 2.93E−01 | 0.00 |
| Gpr137 | 2.06 | 0.00 | 0.06 | 0.00 | 1 | 0.01 | 9.61E−01 | −0.18 |
| Ncoa5 | 2.06 | 0.00 | 0.06 | 0.00 | 1 | 0.24 | 3.02E−01 | −0.10 |
| Mapkapk5 | 2.06 | 0.00 | 0.06 | 0.00 | 1 | −0.10 | 6.31E−01 | −0.06 |
| Auh | 2.05 | 0.00 | 0.05 | 0.00 | 1 | 0.16 | 7.47E−01 | −0.32 |
| Gpx3 | 2.05 | 0.00 | 0.05 | 0.00 | 1 | −0.06 | 5.82E−01 | 0.00 |
| Tmem116 | 2.05 | 0.03 | 0.00 | 0.00 | 1 | −0.01 | 9.16E−01 | 0.00 |
| Hint1 | 2.05 | 0.00 | 0.05 | 0.00 | 1 | −0.11 | 5.88E−01 | −0.01 |
| Mtmr3 | 2.05 | 0.00 | 0.05 | 0.00 | 1 | −0.04 | 9.16E−01 | −0.20 |
| Ube2l3 | 2.05 | 0.00 | 0.05 | 0.00 | 1 | 0.06 | 9.16E−01 | 0.13 |
| Il23r | 2.05 | 0.00 | 0.05 | 0.00 | 1 | 0.01 | 9.16E−01 | −0.07 |
| Asb2 | 2.05 | 0.46 | 1.12 | 0.00 | 0 | 0.93 | 8.67E−11 | 0.94 |
| Snx32 | 2.05 | 0.00 | 0.05 | 0.00 | 1 | 0.15 | 3.44E−01 | 0.02 |
| Fgfr1op | 2.04 | 0.00 | 0.04 | 0.00 | 1 | −0.14 | 6.79E−01 | 0.01 |
| Ccl2 | 2.04 | 0.00 | 0.04 | 0.00 | 1 | −0.04 | 7.20E−01 | 0.00 |
| Slc16a10 | 2.04 | 0.00 | 0.04 | 0.00 | 1 | 0.04 | 9.16E−01 | −0.12 |
| Batf | 2.04 | 0.19 | 0.76 | 0.90 | 0 | −0.99 | 1.85E−09 | −0.11 |
| Trpt1 | 2.04 | 0.00 | 0.04 | 0.00 | 1 | 0.09 | 7.33E−01 | 0.28 |
| Jazf1 | 2.04 | 0.00 | 0.04 | 0.00 | 1 | −0.11 | 2.74E−01 | 0.00 |
| Tubd1 | 2.04 | 0.00 | 0.04 | 0.00 | 1 | −0.01 | 9.16E−01 | −0.06 |
| Rtel1 | 2.04 | 0.00 | 0.04 | 0.00 | 1 | 0.11 | 6.34E−01 | 0.22 |
| Srm | 2.04 | 0.00 | 0.08 | 1.96 | 0 | −0.02 | 9.16E−01 | −0.37 |
| Thada | 2.03 | 0.00 | 0.03 | 0.00 | 1 | −0.09 | 8.72E−01 | 0.43 |
| Tppp | 2.03 | 0.00 | 0.03 | 0.00 | 1 | 0.02 | 9.16E−01 | 0.18 |
| Ppp4c | 2.03 | 0.16 | 0.24 | 1.47 | 0 | 0.12 | 6.55E−01 | −0.24 |
| Per3 | 2.03 | 0.00 | 0.03 | 0.00 | 1 | 0.09 | 7.22E−01 | 0.05 |
| Hormad2 | 2.03 | 0.00 | 0.03 | 0.00 | 1 | −0.04 | 6.91E−01 | 0.00 |
| Gm20186 | 2.03 | 0.45 | 1.13 | 0.00 | 0 | −1.00 | 1.94E−06 | 0.19 |
| Ccdc122 | 2.02 | 0.00 | 0.02 | 0.00 | 1 | 0.05 | 7.03E−01 | −0.06 |
| Fasl | 2.02 | 0.19 | 1.65 | 0.00 | 0 | −1.32 | 8.53E−15 | −0.45 |
| Sh2d2a | 2.02 | 0.24 | 1.54 | 0.00 | 0 | −0.82 | 1.67E−09 | −0.73 |
| Atp6v0a1 | 2.02 | 0.00 | 0.02 | 0.00 | 1 | −0.03 | 8.25E−01 | 0.00 |
| Fosl1 | 2.01 | 0.00 | 0.01 | 0.00 | 1 | −0.01 | 9.16E−01 | 0.00 |
| Itgb8 | 2.01 | 0.00 | 0.01 | 0.00 | 1 | −0.02 | 9.16E−01 | 0.00 |
| Amz1 | 2.01 | 0.00 | 0.01 | 0.00 | 1 | 0.05 | 6.82E−01 | 0.15 |
| Cxcl5 | 2.01 | 0.00 | 0.01 | 0.00 | 1 | −0.01 | 9.16E−01 | 0.00 |
| Nkx2-3 | 2.01 | 0.00 | 0.01 | 0.00 | 1 | −0.01 | 9.16E−01 | 0.00 |
| Thbs3 | 2.01 | 0.00 | 0.01 | 0.00 | 1 | −0.01 | 9.16E−01 | 0.00 |
| Pf4 | 2.01 | 0.00 | 0.01 | 0.00 | 1 | 0.03 | 8.56E−01 | −0.06 |
| Ephx4 | 2.01 | 0.00 | 0.01 | 0.00 | 1 | −0.01 | 9.16E−01 | 0.00 |
| Osmr | 2.01 | 0.00 | 0.01 | 0.00 | 1 | 0.00 | 9.16E−01 | 0.00 |
| Ccl11 | 2.01 | 0.00 | 0.01 | 0.00 | 1 | −0.01 | 9.16E−01 | 0.00 |
| Fndc4 | 2.01 | 0.00 | 0.01 | 0.00 | 1 | −0.01 | 9.16E−01 | 0.00 |
| Ccl7 | 2.01 | 0.00 | 0.01 | 0.00 | 1 | −0.01 | 9.16E−01 | 0.00 |
| Ptrf | 2.01 | 0.00 | 0.01 | 0.00 | 1 | −0.01 | 9.16E−01 | 0.00 |
| Mei1 | 2.00 | 0.00 | 0.00 | 0.00 | 1 | 0.00 | 9.16E−01 | 0.00 |
| Lamb1 | 2.00 | 0.00 | 0.00 | 0.00 | 1 | 0.00 | 9.16E−01 | 0.00 |

| Gene | LPL-9_Geno_FDR | LPL-2_logFC | LPL-2_FDR | LPL-2_Geno_logFC | LPL-2_Geno_FDR |
|---|---|---|---|---|---|
| Gpr18 | 8.21E−02 | 0.30 | 2.12E−06 | −0.87 | 1.82E−10 |
| Gzmb | 1.00E+00 | 0.46 | 9.03E−09 | −0.74 | 1.86E−06 |
| Itgb1 | 8.72E−01 | 1.75 | 1.74E−200 | 0.47 | 8.89E−03 |
| Ccr3 | 1.00E+00 | 0.00 | 8.83E−01 | 0.02 | 9.32E−01 |
| Gzma | 2.40E−10 | 1.16 | 4.89E−41 | −1.73 | 5.05E−18 |
| Il22 | 1.00E+00 | −1.76 | 3.12E−27 | 0.80 | 2.21E−06 |
| Zfp36l2 | 6.35E−03 | 0.22 | 9.29E−06 | −0.76 | 2.01E−14 |
| Zbtb38 | 1.00E+00 | −0.09 | 3.49E−01 | −0.17 | 5.91E−01 |
| Cd74 | 1.00E+00 | 0.29 | 3.04E−03 | 0.49 | 3.79E−02 |
| Ccr5 | 1.00E+00 | −0.08 | 4.32E−01 | −0.70 | 1.48E−08 |
| Cd160 | 4.54E−04 | 0.24 | 1.01E−02 | −1.39 | 5.51E−17 |
| Map3k8 | 1.00E+00 | 0.27 | 4.85E−06 | 0.10 | 9.09E−01 |
| Tnfsf8 | 4.04E−01 | −0.74 | 3.11E−27 | 0.87 | 1.14E−10 |
| Ifitm1 | 1.00E+00 | 3.16 | 1.00E−167 | 1.21 | 2.18E−07 |
| Nfkbiz | 1.00E+00 | −0.17 | 1.07E−03 | 0.20 | 3.50E−01 |
| Fosl2 | 1.00E+00 | −0.70 | 4.61E−36 | 0.14 | 6.91E−01 |
| Crem | 1.00E+00 | −0.94 | 1.07E−66 | 0.46 | 3.59E−05 |
| Ccdc85b | 1.00E+00 | 0.06 | 4.76E−01 | −0.25 | 1.73E−01 |
| Fos | 8.71E−01 | −1.04 | 1.20E−32 | 0.53 | 4.09E−02 |
| Gpr183 | 1.00E+00 | 0.21 | 5.21E−06 | −0.09 | 8.27E−01 |
| S100a4 | 8.76E−01 | 1.53 | 2.23E−210 | 0.10 | 6.18E−01 |
| 1110008f13rik | 1.00E+00 | 0.32 | 2.97E−13 | 0.06 | 9.31E−01 |
| Lsp1 | 1.00E+00 | 0.61 | 9.93E−121 | −0.20 | 7.10E−04 |
| Litaf | 9.15E−01 | −0.23 | 6.76E−04 | 0.29 | 1.70E−01 |
| Cd7 | 2.19E−05 | 0.76 | 1.54E−22 | −1.10 | 2.81E−13 |
| Dusp2 | 2.07E−01 | 0.22 | 2.20E−03 | −0.21 | 5.56E−01 |
| Plac8 | 8.71E−02 | 0.32 | 8.98E−09 | −1.00 | 1.74E−25 |
| H1f0 | 9.15E−01 | 0.39 | 4.24E−09 | −0.57 | 2.68E−04 |
| S1pr1 | 9.15E−01 | 0.88 | 1.61E−61 | −0.15 | 6.60E−01 |
| Ncf4 | 1.00E+00 | 0.62 | 2.05E−37 | −0.29 | 6.51E−02 |
| Smim3 | 1.00E+00 | −0.11 | 1.78E−01 | 0.23 | 4.28E−01 |
| Tesc | 1.00E+00 | −0.34 | 4.70E−12 | 0.04 | 1.00E+00 |
| Rbms1 | 1.00E+00 | 0.29 | 7.80E−10 | −0.15 | 5.25E−01 |
| Lpxn | 1.00E+00 | −0.04 | 6.22E−01 | −0.21 | 9.69E−02 |
| Tnfrsf9 | 9.15E−01 | −0.34 | 8.09E−06 | 0.13 | 9.01E−01 |
| Pmm1 | 1.00E+00 | 0.14 | 5.91E−02 | −0.09 | 9.31E−01 |
| Tob2 | 1.00E+00 | −0.75 | 9.68E−37 | −0.03 | 1.00E+00 |
| Ifng | 9.15E−01 | −0.11 | 4.38E−01 | 0.25 | 4.83E−01 |
| Cd226 | 1.00E+00 | 0.22 | 3.24E−04 | −0.35 | 7.33E−02 |
| Ctsw | 1.00E+00 | 0.41 | 2.35E−33 | −0.40 | 1.07E−08 |
| Itga4 | 9.15E−01 | 0.40 | 1.49E−15 | −0.41 | 2.71E−03 |
| Cd28 | 1.00E+00 | 0.08 | 6.50E−02 | 0.29 | 5.47E−05 |
| Klf2 | 5.50E−01 | 1.25 | 4.15E−91 | −0.23 | 2.36E−01 |
| Ly6c2 | 8.72E−01 | 1.60 | 2.37E−58 | 0.29 | 6.52E−01 |
| Ccr9 | 5.15E−07 | −0.39 | 8.08E−05 | −0.87 | 4.80E−06 |
| Ets1 | 1.00E+00 | −0.13 | 1.64E−05 | −0.16 | 9.20E−02 |
| Il7r | 8.46E−01 | 0.14 | 5.94E−03 | −0.35 | 7.68E−04 |
| Ptpn22 | 6.92E−01 | −0.12 | 2.54E−03 | −0.03 | 1.00E+00 |
| Gm12840 | 1.00E+00 | 0.09 | 3.27E−01 | −0.59 | 7.30E−07 |
| Zfp36l1 | 8.94E−01 | −0.35 | 2.80E−10 | −0.11 | 7.89E−01 |
| Atxn2l | 1.00E+00 | 0.01 | 8.67E−01 | 0.02 | 1.00E+00 |
| Pde4b | 1.00E+00 | −0.05 | 4.58E−01 | −0.04 | 1.00E+00 |
| Selp | 1.00E+00 | 0.01 | 5.37E−01 | 0.03 | 5.25E−01 |
| Anxa6 | 1.00E+00 | 0.48 | 6.74E−49 | 0.01 | 1.00E+00 |
| Peli1 | 1.00E+00 | 0.00 | 9.94E−01 | 0.18 | 2.36E−01 |
| Odc1 | 1.00E+00 | −1.00 | 1.42E−103 | 0.78 | 1.42E−15 |
| Pglyrp1 | 1.00E+00 | 1.21 | 4.44E−123 | −0.34 | 1.61E−02 |
| Pim3 | 1.00E+00 | −0.48 | 3.57E−14 | 0.29 | 2.16E−01 |
| Pim1 | 7.90E−02 | −0.11 | 1.68E−02 | 0.38 | 1.17E−07 |
| Mt1 | 9.15E−01 | 0.69 | 2.54E−19 | −0.66 | 4.42E−03 |
| Stat3 | 1.00E+00 | −0.26 | 1.03E−07 | 0.19 | 3.44E−01 |
| Stat5a | 1.00E+00 | −0.11 | 1.64E−01 | 0.16 | 7.24E−01 |
| Vmp1 | 6.99E−01 | −0.01 | 9.62E−01 | −0.27 | 6.92E−02 |
| Ier2 | 1.00E+00 | 0.21 | 2.69E−07 | −0.03 | 1.00E+00 |
| Tnip1 | 1.00E+00 | 0.13 | 3.16E−02 | 0.14 | 6.52E−01 |
| Timp1 | 1.00E+00 | 0.00 | 5.52E−01 | 0.00 | 1.00E+00 |
| Amdhd1 | 1.00E+00 | 0.01 | 6.41E−01 | −0.02 | 1.00E+00 |
| Rora | 1.00E+00 | 0.12 | 9.23E−03 | 0.16 | 2.68E−01 |
| Gm5111 | 1.00E+00 | 0.01 | 5.23E−01 | −0.04 | 5.70E−01 |
| Fermt3 | 1.00E+00 | 0.19 | 2.35E−04 | −0.31 | 2.58E−02 |
| Dap | 7.71E−01 | 0.15 | 1.25E−02 | −0.34 | 3.38E−02 |
| Hist1h1c | 1.00E+00 | 0.52 | 2.52E−16 | −0.37 | 9.01E−02 |
| Cfl1 | 2.14E−01 | 0.15 | 5.96E−13 | 0.02 | 1.00E+00 |
| Ipmk | 1.00E+00 | −0.36 | 1.89E−08 | 0.16 | 6.84E−01 |
| Klrd1 | 2.07E−01 | 1.28 | 3.41E−111 | −0.26 | 1.92E−01 |
| Gfi1 | 9.92E−01 | −0.01 | 9.97E−01 | −0.11 | 8.05E−01 |
| S100a6 | 1.00E+00 | 1.20 | 1.69E−261 | 0.06 | 8.25E−01 |
| Rorc | 1.00E+00 | −0.11 | 3.02E−04 | −0.02 | 9.31E−01 |
| Stat4 | 1.00E+00 | −0.28 | 2.49E−08 | 0.25 | 1.02E−01 |
| Fdps | 1.00E+00 | −0.09 | 2.97E−01 | 0.06 | 1.00E+00 |
| Gpr65 | 1.00E+00 | −0.33 | 1.74E−07 | 0.02 | 1.00E+00 |
| Rapgef6 | 1.00E+00 | −0.02 | 8.37E−01 | −0.07 | 9.35E−01 |
| Tmem160 | 1.00E+00 | 0.27 | 2.30E−12 | −0.02 | 1.00E+00 |
| Rasgrp1 | 1.00E+00 | −0.39 | 6.41E−14 | −0.07 | 9.31E−01 |
| Pnkd | 9.15E−01 | 0.08 | 2.94E−01 | −0.11 | 8.44E−01 |
| Ccnd3 | 1.00E+00 | 0.41 | 1.47E−21 | 0.28 | 1.64E−02 |
| Panx1 | 1.00E+00 | 0.20 | 5.88E−04 | −0.06 | 1.00E+00 |
| Thap11 | 9.15E−01 | 0.15 | 1.28E−02 | −0.22 | 3.96E−01 |
| Lysmd2 | 1.00E+00 | −0.01 | 8.99E−01 | −0.34 | 8.45E−02 |
| Ubac2 | 1.00E+00 | 0.03 | 7.34E−01 | −0.21 | 2.85E−01 |
| Lgals3 | 9.15E−01 | 1.40 | 2.98E−233 | 0.23 | 2.11E−02 |
| Cyb5r4 | 1.00E+00 | 0.32 | 4.83E−11 | −0.07 | 9.31E−01 |
| Upf3a | 1.00E+00 | 0.12 | 9.45E−02 | −0.39 | 1.19E−02 |
| Gm10076 | 1.00E+00 | −0.09 | 8.50E−02 | −1.04 | 8.24E−36 |
| Ccdc69 | 1.00E+00 | 0.08 | 3.35E−01 | −0.10 | 8.77E−01 |
| Ccl5 | 3.77E−04 | 0.81 | 6.76E−48 | −1.13 | 1.94E−20 |
| Gramd3 | 9.26E−01 | 0.42 | 2.99E−26 | −0.21 | 5.32E−02 |
| Errfi1 | 1.00E+00 | −0.25 | 4.81E−04 | 0.26 | 2.86E−01 |
| Itgae | 2.78E−03 | 0.27 | 2.71E−04 | −0.82 | 6.06E−08 |
| Il2ra | 1.00E+00 | 0.03 | 7.88E−01 | 0.22 | 5.25E−01 |
| Gpx4 | 1.00E+00 | −0.35 | 2.78E−26 | 0.11 | 5.10E−01 |
| Ndfip1 | 1.00E+00 | −0.14 | 2.09E−03 | 0.14 | 4.52E−01 |
| Syt11 | 1.00E+00 | 0.10 | 2.17E−01 | −0.05 | 1.00E+00 |
| Dph5 | 9.15E−01 | −0.02 | 7.93E−01 | 0.11 | 8.35E−01 |
| Slc39a8 | 9.15E−01 | −0.07 | 2.01E−01 | −0.08 | 7.03E−01 |
| Rnf187 | 1.00E+00 | −0.11 | 5.11E−02 | 0.02 | 1.00E+00 |
| Fam53b | 1.00E+00 | 0.19 | 2.10E−03 | 0.04 | 1.00E+00 |
| Prdx5 | 9.15E−01 | −0.07 | 2.29E−01 | 0.11 | 6.80E−01 |
| Ccrl2 | 5.56E−01 | −0.15 | 8.31E−02 | 0.24 | 4.99E−01 |
| Ep300 | 1.00E+00 | 0.06 | 5.04E−01 | −0.13 | 7.40E−01 |
| Hdac7 | 1.00E+00 | 0.02 | 7.59E−01 | −0.28 | 7.88E−02 |
| Cd6 | 1.00E+00 | −0.05 | 4.17E−01 | −0.23 | 5.53E−02 |
| Traf3ip2 | 7.75E−01 | 0.08 | 2.19E−01 | −0.29 | 9.01E−02 |
| Gnb2 | 1.00E+00 | 0.17 | 7.24E−07 | −0.20 | 3.37E−02 |
| Tet2 | 1.00E+00 | −0.17 | 1.02E−02 | 0.11 | 8.71E−01 |
| Fam189b | 1.00E+00 | −0.09 | 2.93E−01 | −0.08 | 9.31E−01 |
| Skap2 | 1.00E+00 | 0.04 | 6.97E−01 | −0.06 | 1.00E+00 |
| Lgals1 | 8.36E−01 | 1.11 | 0.00E+00 | 0.30 | 1.31E−09 |
| Socs1 | 1.00E+00 | −0.29 | 3.38E−06 | 0.34 | 6.39E−02 |
| Itgav | 9.15E−01 | −0.36 | 1.44E−09 | 0.30 | 1.07E−01 |
| Dnajb4 | 1.00E+00 | −0.31 | 3.77E−06 | 0.34 | 6.08E−02 |
| Cdkn1a | 1.00E+00 | −0.69 | 6.69E−31 | 0.80 | 2.00E−10 |
| Arhgef2 | 1.00E+00 | 0.16 | 1.31E−02 | −0.19 | 5.25E−01 |
| Dnmt3a | 1.00E+00 | −0.06 | 4.56E−01 | −0.08 | 9.31E−01 |
| Jund | 8.04E−06 | −0.12 | 1.18E−03 | 0.68 | 6.22E−42 |
| Scamp3 | 1.00E+00 | 0.09 | 1.62E−01 | −0.09 | 8.64E−01 |
| Tmem30a | 6.80E−01 | 0.14 | 1.32E−02 | −0.35 | 7.50E−03 |
| Ash1l | 1.00E+00 | −0.03 | 7.62E−01 | 0.02 | 1.00E+00 |
| Lsm14a | 1.00E+00 | 0.10 | 1.22E−01 | −0.15 | 6.13E−01 |
| Slain2 | 1.00E+00 | 0.02 | 8.45E−01 | −0.14 | 6.99E−01 |
| Muc1 | 1.00E+00 | 0.11 | 9.34E−02 | 0.02 | 1.00E+00 |
| Adamts14 | 1.00E+00 | 0.00 | 7.34E−01 | 0.00 | 1.00E+00 |
| Ptk2b | 1.00E+00 | 0.02 | 8.39E−01 | −0.06 | 1.00E+00 |
| Stk11 | 1.00E+00 | −0.34 | 1.06E−11 | 0.11 | 8.41E−01 |
| Ccr2 | 1.00E+00 | 0.17 | 2.17E−03 | 0.11 | 7.02E−01 |
| Itgal | 4.62E−01 | 0.06 | 5.02E−01 | 0.06 | 9.60E−01 |
| Elf1 | 9.15E−01 | 0.01 | 9.24E−01 | 0.01 | 1.00E+00 |
| Ptprc | 1.00E+00 | 0.20 | 4.24E−12 | −0.17 | 6.25E−02 |
| Rbx1 | 1.00E+00 | 0.06 | 1.77E−01 | −0.07 | 8.72E−01 |
| Traf3ip3 | 9.74E−01 | 0.27 | 6.92E−07 | −0.44 | 1.42E−03 |
| Ccdc88b | 1.00E+00 | 0.10 | 2.07E−01 | 0.12 | 8.72E−01 |
| Tsen34 | 1.00E+00 | −0.01 | 8.73E−01 | −0.08 | 8.78E−01 |
| Ay036118 | 1.00E+00 | −0.29 | 8.27E−06 | −1.15 | 3.92E−23 |
| Il24 | | | | | |
| H2-q10 | 1.00E+00 | −0.07 | 5.33E−01 | 0.34 | 3.05E−02 |
| Scand1 | 1.00E+00 | 0.16 | 8.97E−05 | −0.17 | 2.65E−01 |
| Tab2 | 1.00E+00 | 0.14 | 3.65E−02 | 0.09 | 9.31E−01 |
| Nfkb1 | 1.00E+00 | −0.15 | 5.50E−03 | 0.14 | 5.92E−01 |
| Rusc1 | 1.00E+00 | 0.13 | 5.06E−02 | −0.09 | 9.12E−01 |
| Esyt1 | 1.00E+00 | 0.48 | 8.09E−24 | −0.13 | 6.89E−01 |
| Commd7 | 9.96E−01 | 0.04 | 6.83E−01 | −0.10 | 9.01E−01 |
| Rfng | 1.00E+00 | 0.01 | 9.19E−01 | −0.19 | 4.61E−01 |
| Rpl27-ps3 | 2.26E−01 | −0.28 | 1.77E−10 | −1.05 | 8.09E−50 |
| Gigyf1 | 1.00E+00 | −0.10 | 1.28E−01 | −0.11 | 7.49E−01 |
| Rgcc | 6.43E−01 | 0.04 | 7.34E−01 | 1.71 | 1.89E−45 |
| Rpl3 | 9.15E−01 | −0.02 | 4.86E−01 | −0.15 | 2.99E06 |
| Mrpl20 | 1.00E+00 | 0.16 | 1.55E−03 | −0.03 | 1.00E+00 |
| Gm2a | 1.00E+00 | −0.21 | 3.51E−05 | 0.15 | 5.25E−01 |
| Mus81 | 1.00E+00 | 0.13 | 1.54E−02 | −0.03 | 1.00E+00 |
| Lpp | 1.00E+00 | 0.06 | 3.71E−01 | −0.18 | 5.12E−01 |
| Chp1 | 9.15E−01 | 0.16 | 4.42E−03 | 0.00 | 1.00E+00 |
| Mob4 | 1.00E+00 | 0.11 | 5.09E−02 | 0.10 | 8.35E−01 |
| Lamp1 | 1.00E+00 | −0.06 | 4.76E−01 | −0.03 | 1.00E+00 |
| Phf5a | 1.00E+00 | 0.16 | 2.65E−03 | −0.24 | 1.51E−01 |
| Fnip1 | 1.00E+00 | 0.11 | 8.89E−02 | −0.01 | 1.00E+00 |
| Calm3 | 1.00E+00 | −0.05 | 4.72E−01 | 0.08 | 8.45E−01 |
| Lrp10 | 1.00E+00 | 0.13 | 2.73E−02 | −0.14 | 5.81E−01 |
| Stat5b | 1.00E+00 | −0.12 | 1.09E−01 | 0.02 | 1.00E+00 |
| Bach2 | 1.00E+00 | −0.04 | 4.97E−01 | −0.04 | 1.00E+00 |
| Prkab1 | 1.00E+00 | −0.02 | 8.52E−01 | 0.27 | 1.41E−01 |
| Hvcn1 | 9.15E−01 | −0.18 | 5.47E−03 | 0.21 | 4.56E−01 |
| Tmem150a | 1.00E+00 | 0.01 | 5.10E−01 | −0.01 | 1.00E+00 |
| Nfkbia | 1.00E+00 | 0.22 | 5.16E−08 | 0.27 | 1.43E−03 |
| Ifitm3 | 1.00E+00 | 1.07 | 9.58E−66 | 0.02 | 1.00E+00 |
| Egfp_ires_il23r | | | | | |
| Bad | 1.00E+00 | 0.15 | 1.86E−02 | −0.05 | 1.00E+00 |
| Zbtb46 | 1.00E+00 | 0.10 | 2.77E−02 | −0.08 | 8.94E−01 |
| Cacna1s | 1.00E+00 | −0.05 | 2.78E−01 | −0.01 | 1.00E+00 |
| Vamp3 | 1.00E+00 | 0.15 | 1.23E−02 | 0.05 | 1.00E+00 |
| Fyn | 1.00E+00 | −0.06 | 4.94E−01 | −0.06 | 9.32E−01 |
| Stx6 | 1.00E+00 | −0.25 | 1.19E−05 | −0.05 | 1.00E+00 |
| Ikzf3 | 1.00E+00 | −0.24 | 5.66E−04 | 0.17 | 6.73E−01 |
| Mgat2 | 1.00E+00 | 0.17 | 1.12E−03 | 0.16 | 5.10E−01 |
| Serpinb9 | 9.50E−01 | 0.26 | 8.45E−06 | −0.35 | 9.55E−03 |
| Dennd1b | 9.15E−01 | −0.02 | 8.68E−01 | −0.16 | 5.86E−01 |
| Bloc1s4 | 1.00E+00 | 0.00 | 9.85E−01 | 0.18 | 5.25E−01 |
| Anxa1 | 1.00E+00 | 1.20 | 1.76E−91 | 0.79 | 2.26E−09 |
| Smad3 | 1.00E+00 | 0.15 | 1.68E−02 | 0.01 | 1.00E+00 |
| Tmem258 | 1.00E+00 | −0.02 | 7.37E−01 | 0.06 | 9.31E−01 |
| Lmna | 8.60E−04 | 0.20 | 1.46E−02 | 1.94 | 1.16E−69 |
| Sp140 | 1.00E+00 | 0.15 | 2.19E−02 | −0.23 | 3.43E−01 |
| Actn2 | 1.00E+00 | 0.15 | 3.07E−02 | −0.56 | 2.95E−05 |
| Acbd6 | 1.00E+00 | 0.01 | 8.54E−01 | −0.20 | 4.30E−01 |
| Ahnak | 1.00E+00 | 0.50 | 1.39E−39 | 0.16 | 3.54E−01 |
| Arpc2 | 1.00E+00 | 0.16 | 4.07E−15 | −0.02 | 9.72E−01 |
| Erp29 | 1.00E+00 | 0.19 | 6.28E−04 | −0.03 | 1.00E+00 |
| Nhp2l1 | 1.00E+00 | −0.23 | 1.13E−09 | 0.19 | 1.40E−01 |
| Ctbp1 | 1.00E+00 | 0.03 | 6.83E−01 | −0.35 | 1.72E−03 |
| Gba | 1.00E+00 | 0.11 | 9.45E−02 | −0.20 | 4.53E−01 |
| Lztfl1 | 9.59E−01 | −0.32 | 1.88E−07 | −0.56 | 2.81E−04 |
| Ptpn2 | 1.00E+00 | 0.04 | 6.83E−01 | −0.13 | 7.03E−01 |
| Usp36 | 1.00E+00 | −0.15 | 4.22E−02 | −0.16 | 6.31E−01 |
| Grb7 | 9.96E−01 | −0.19 | 4.07E−04 | 0.15 | 5.18E−01 |
| Fndc3a | 9.96E−01 | −0.03 | 7.34E−01 | 0.08 | 9.31E−01 |
| Mt2 | 9.78E−01 | 0.41 | 1.92E−10 | −0.40 | 3.25E−01 |
| Dctn4 | 9.15E−01 | 0.03 | 7.34E−01 | 0.05 | 1.00E+00 |
| Nusap1 | 1.00E+00 | −0.43 | 6.53E−13 | 0.12 | 4.14E−01 |
| Rbm22 | 1.00E+00 | 0.16 | 1.75E−03 | −0.19 | 3.93E−01 |
| Crip1 | 1.00E+00 | 1.16 | 3.21E−287 | 0.54 | 9.93E−22 |
| Anp32b | 1.00E+00 | −0.20 | 1.65E−05 | 0.14 | 5.25E−01 |
| Aurkaip1 | 1.00E+00 | 0.04 | 6.22E−01 | 0.04 | 1.00E+00 |
| Ripk2 | 1.00E+00 | 0.21 | 1.59E−04 | −0.15 | 6.84E−01 |
| Nubp1 | 1.00E+00 | 0.12 | 6.45E−02 | −0.28 | 1.17E−01 |
| Camta1 | 1.00E+00 | −0.05 | 5.65E−01 | −0.08 | 9.33E−01 |
| Ube2j2 | 1.00E+00 | 0.05 | 4.56E−01 | −0.04 | 1.00E+00 |
| Kif3b | 9.15E−01 | 0.00 | 9.36E−01 | −0.09 | 7.23E−01 |
| Brap | 1.00E+00 | −0.08 | 3.33E−01 | −0.04 | 1.00E+00 |
| Gm8096 | | | | | |
| Ermn | 1.00E+00 | −0.10 | 4.16E−01 | −0.13 | 9.31E−01 |
| Plagl2 | 1.00E+00 | −0.05 | 5.51E−01 | −0.10 | 8.23E−01 |
| Plek | 1.00E+00 | 0.44 | 1.16E−12 | 0.01 | 1.00E+00 |
| D030056l22rik | 1.00E+00 | −0.29 | 3.39E−06 | 0.22 | 3.86E−01 |
| Ccr1 | 7.11E−01 | −0.20 | 1.53E−03 | 0.17 | 4.25E−01 |
| Krtcap2 | 5.81E−03 | 0.14 | 3.21E−06 | 0.31 | 3.46E−07 |
| Sh2b1 | 1.00E+00 | 0.01 | 8.91E−01 | −0.13 | 8.16E−01 |
| Abi3 | 9.96E−01 | −0.51 | 3.25E−16 | −0.12 | 8.68E−01 |
| Ccnl2 | 1.00E+00 | 0.06 | 4.38E−01 | −0.02 | 1.00E+00 |
| Kbtbd7 | 1.00E+00 | 0.03 | 4.57E−01 | 0.06 | 8.26E−01 |
| Csf2 | 1.00E+00 | 0.07 | 7.09E−01 | 0.43 | 1.96E−02 |
| Pycard | 9.15E−01 | 0.82 | 8.39E−90 | −0.55 | 3.15E−10 |
| Dock7 | 1.00E+00 | 0.00 | 6.83E−01 | 0.00 | 1.00E+00 |
| Herpud1 | 2.44E−01 | 0.06 | 4.49E−01 | 0.22 | 1.99E−01 |
| Arf6 | 1.00E+00 | 0.12 | 1.50E−03 | 0.22 | 1.95E−02 |
| Zyx | 1.00E+00 | 0.87 | 1.51E−71 | −0.22 | 2.86E−01 |
| Zfp91 | 1.00E+00 | −0.04 | 6.57E−01 | 0.09 | 9.35E−01 |
| Fam171b | 1.00E+00 | 0.02 | 5.51E−01 | 0.02 | 1.00E+00 |
| Maml2 | 1.00E+00 | 0.12 | 7.23E−02 | 0.02 | 1.00E+00 |
| Ifngr2 | 1.00E+00 | −0.22 | 6.48E−06 | 0.04 | 1.00E+00 |
| Bhlhe40 | 9.15E−01 | 0.12 | 1.44E−04 | 0.18 | 7.29E−03 |
| Cisd1 | 1.00E+00 | −0.01 | 8.15E−01 | 0.07 | 9.58E−01 |
| Zfp420 | 7.11E−01 | −0.01 | 7.62E−01 | 0.02 | 1.00E+00 |
| Zc3h12a | 1.00E+00 | 0.11 | 6.00E−02 | 0.46 | 2.45E−06 |
| Plcg2 | 9.15E−01 | 0.08 | 1.03E−02 | −0.07 | 8.19E−01 |
| 2310036o22rik | 1.00E+00 | 0.03 | 6.28E−01 | −0.11 | 6.52E−01 |
| Eif3c | 1.00E+00 | 0.11 | 4.69E−02 | −0.06 | 9.31E−01 |
| Slc15a3 | 1.00E+00 | −0.18 | 1.93E−02 | −0.01 | 1.00E+00 |
| Acap3 | 1.00E+00 | 0.10 | 4.53E−02 | 0.13 | 6.52E−01 |
| Tef | 1.00E+00 | 0.02 | 7.34E−01 | 0.18 | 4.17E−01 |
| Emp3 | 1.00E+00 | 0.93 | 4.59E−187 | 0.05 | 9.01E−01 |
| Ssr2 | 1.00E+00 | 0.06 | 5.51E−01 | −0.06 | 9.83E−01 |
| Usp1 | 1.00E+00 | −0.10 | 1.51E−01 | 0.18 | 5.37E−01 |
| Rpl10-ps3 | 1.00E+00 | −0.25 | 1.60E−07 | −0.89 | 5.90E−25 |
| Plcxd2 | 1.00E+00 | −0.30 | 1.20E−06 | −0.42 | 9.75E−03 |
| Adcy7 | 1.00E+00 | 0.03 | 7.34E−01 | −0.06 | 9.85E−01 |
| Irf1 | 1.00E+00 | 0.07 | 4.01E−01 | 0.43 | 1.05E−03 |
| Aamp | 1.00E+00 | −0.10 | 7.72E−02 | 0.06 | 9.31E−01 |
| Gm8730 | 5.54E−06 | 0.05 | 1.49E−01 | −0.78 | 1.16E−56 |
| Zgpat | 1.00E+00 | 0.03 | 6.95E−01 | −0.18 | 5.25E−01 |
| St13 | 9.15E−01 | −0.30 | 3.36E−10 | 0.29 | 4.51E−02 |
| Cdc42se2 | 9.52E−01 | −0.01 | 8.95E−01 | −0.06 | 9.35E−01 |
| Anxa2 | 9.15E−01 | 0.69 | 3.68E−70 | 0.35 | 1.11E−04 |
| Cpsf3l | 1.00E+00 | 0.09 | 2.85E−01 | −0.04 | 1.00E+00 |
| Gm2000 | 8.76E−01 | −0.23 | 7.20E−07 | −0.84 | 1.60E−23 |
| Trim46 | 1.00E+00 | 0.08 | 4.53E−02 | −0.06 | 9.31E−01 |
| Dusp16 | 1.00E+00 | 0.02 | 6.23E−01 | −0.03 | 1.00E+00 |
| Zmat5 | 1.00E+00 | 0.05 | 5.51E−01 | −0.04 | 1.00E+00 |
| Ptprk | 1.00E+00 | 0.06 | 5.33E−01 | −0.18 | 5.86E−01 |
| Zdhhc7 | 8.47E−01 | 0.15 | 1.69E−02 | 0.13 | 7.72E−01 |
| Il2 | 8.72E−01 | −0.23 | 8.82E−04 | 0.01 | 1.00E+00 |
| Msto1 | 1.00E+00 | 0.02 | 7.46E−01 | −0.14 | 5.96E−01 |
| Il10 | 1.00E+00 | −0.26 | 2.62E−05 | −0.02 | 1.00E+00 |
| Sbno2 | 1.00E+00 | 0.03 | 6.75E−01 | −0.11 | 6.35E−01 |
| Snx17 | 1.00E+00 | −0.02 | 8.03E−01 | −0.26 | 1.84E−01 |
| Kif11 | 1.00E+00 | −0.13 | 8.65E−06 | −0.10 | 3.77E−01 |
| Ndfip2 | 1.00E+00 | 0.07 | 3.33E−01 | −0.07 | 9.31E−01 |
| Card9 | 9.96E−01 | −0.01 | 7.34E−01 | 0.02 | 5.25E−01 |
| Lsm4 | 9.15E−01 | 0.02 | 7.43E−01 | −0.12 | 5.91E−01 |
| Ormdl3 | 9.96E−01 | −0.22 | 4.71E−04 | −0.04 | 1.00E+00 |
| S100a10 | 5.39E−01 | 0.71 | 2.92E−237 | 0.06 | 6.52E−01 |
| Notch2 | 1.00E+00 | −0.14 | 3.79E−02 | 0.02 | 1.00E+00 |
| Senp7 | 1.00E+00 | 0.00 | 9.90E−01 | −0.12 | 8.32E−01 |
| Cd48 | 1.00E+00 | 0.80 | 2.02E−100 | −0.29 | 2.03E−03 |
| Ssbp4 | 1.00E+00 | 0.13 | 1.85E−02 | 0.14 | 5.70E−01 |
| Cul1 | 1.00E+00 | −0.05 | 5.74E−01 | −0.09 | 9.31E−01 |
| Mettl10 | 1.00E+00 | 0.11 | 1.33E−01 | 0.05 | 1.00E+00 |
| Rabep2 | 1.00E+00 | 0.10 | 1.23E−01 | −0.08 | 9.31E−01 |
| Pkig | 4.59E−01 | −0.09 | 4.74E−02 | 0.10 | 7.03E−01 |
| Desi1 | 1.00E+00 | −0.04 | 6.83E−01 | −0.15 | 6.87E−01 |
| Furin | 5.16E−01 | −0.61 | 7.81E−32 | 0.43 | 2.07E−04 |
| Tcof1 | 1.00E+00 | −0.05 | 6.22E−01 | −0.01 | 1.00E+00 |
| Glipr2 | 1.00E+00 | 0.84 | 2.28E−92 | −0.01 | 1.00E+00 |
| Tgfbr3 | 1.00E+00 | −0.05 | 4.97E−01 | −0.05 | 9.06E−01 |
| Tyk2 | 4.66E−01 | 0.07 | 3.28E−01 | −0.03 | 1.00E+00 |
| Kif21b | 1.00E+00 | 0.00 | 9.79E−01 | −0.07 | 9.62E−01 |
| Tufm | 1.00E+00 | 0.02 | 7.86E−01 | 0.01 | 1.00E+00 |
| Zmiz1 | 1.00E+00 | 0.05 | 5.75E−01 | −0.24 | 3.12E−01 |
| Il21 | 1.00E+00 | −0.13 | 8.93E−04 | 0.04 | 8.72E−01 |
| Ttpal | 1.00E+00 | 0.05 | 5.37E−01 | −0.04 | 1.00E+00 |
| Rps6kb1 | 7.31E−01 | −0.10 | 1.94E−01 | 0.03 | 1.00E+00 |
| Camk2g | 1.00E+00 | 0.07 | 4.32E−01 | −0.14 | 7.13E−01 |
| Tom1 | 1.00E+00 | 0.07 | 3.82E−01 | −0.05 | 9.90E−01 |
| Actr1a | 1.00E+00 | −0.03 | 7.34E−01 | −0.16 | 6.52E−01 |
| Rgs14 | 1.00E+00 | 0.03 | 7.34E−01 | −0.04 | 1.00E+00 |
| Ublcp1 | 1.00E+00 | 0.05 | 5.51E−01 | −0.19 | 4.10E−01 |
| Otud3 | 1.00E+00 | 0.03 | 6.85E−01 | −0.11 | 7.40E−01 |
| Tmem180 | 9.15E−01 | −0.01 | 9.00E−01 | −0.02 | 1.00E+00 |
| Ergic1 | 1.00E+00 | −0.17 | 1.27E−03 | 0.16 | 4.65E−01 |
| Aa467197 | 1.00E+00 | 0.97 | 4.55E−86 | 0.14 | 6.47E−01 |
| Tdp2 | 1.00E+00 | 0.03 | 7.93E−01 | 0.27 | 2.02E−01 |
| Rac1 | 1.00E+00 | 0.07 | 7.04E−02 | −0.05 | 9.27E−01 |
| Phlda3 | 1.00E+00 | 0.01 | 9.18E−01 | 0.17 | 3.67E−01 |
| Trmt112 | 1.00E+00 | 0.00 | 9.68E−01 | 0.02 | 1.00E+00 |
| Ccny | 1.00E+00 | −0.07 | 4.44E−01 | −0.11 | 8.67E−01 |
| Slc43a3 | 9.15E−01 | −0.03 | 6.07E−01 | −0.14 | 4.53E−01 |
| Ezh2 | 1.00E+00 | −0.27 | 4.40E−06 | −0.08 | 9.31E−01 |
| Rps14 | 1.00E+00 | −0.06 | 7.47E−06 | 0.00 | 1.00E+00 |
| Spred2 | 1.00E+00 | −0.05 | 4.68E−01 | −0.01 | 1.00E+00 |
| Rn7s2 | | | | | |
| Rn7s1 | | | | | |
| Rpap2 | 1.00E+00 | −0.02 | 7.66E−01 | −0.14 | 5.25E−01 |
| Cit | 1.00E+00 | −0.08 | 4.93E−03 | 0.21 | 3.02E−01 |
| Ascc2 | 1.00E+00 | 0.04 | 6.64E−01 | −0.06 | 9.35E−01 |
| Ksr1 | 1.00E+00 | 0.06 | 2.37E−01 | −0.09 | 8.29E−01 |
| Edem2 | 1.00E+00 | 0.02 | 7.93E−01 | −0.06 | 1.00E+00 |
| Blzf1 | 1.00E+00 | 0.02 | 8.41E−01 | −0.04 | 1.00E+00 |
| Ppp5c | 1.00E+00 | 0.00 | 9.71E−01 | 0.08 | 9.31E−01 |
| Smurf1 | 9.15E−01 | 0.02 | 7.34E−01 | −0.18 | 2.27E−01 |
| Nop9 | 1.00E+00 | −0.01 | 9.08E−01 | −0.23 | 3.48E−01 |
| Slc30a7 | 1.00E+00 | 0.09 | 1.87E−01 | −0.02 | 1.00E+00 |
| Tnfrsf8 | 1.00E+00 | −0.01 | 6.87E−01 | −0.02 | 9.96E−01 |
| Adcy3 | 1.00E+00 | −0.01 | 8.94E−01 | −0.08 | 8.75E−01 |
| Pdcd1 | 1.00E+00 | −0.01 | 9.71E−01 | 0.37 | 1.36E−01 |
| Fen1 | 1.00E+00 | −0.21 | 2.53E−04 | 0.04 | 1.00E+00 |
| Themis | 9.15E−01 | −0.02 | 8.61E−01 | 0.07 | 9.31E−01 |
| Nudt22 | 1.00E+00 | 0.01 | 9.16E−01 | 0.07 | 9.33E−01 |
| Cyth1 | 1.00E+00 | −0.02 | 8.30E−01 | 0.07 | 9.53E−01 |
| Tmbim1 | 9.15E−01 | −0.01 | 9.43E−01 | −0.02 | 1.00E+00 |
| Ndufaf1 | 1.00E+00 | 0.04 | 6.31E−01 | −0.09 | 8.87E−01 |
| Klrc1 | 8.76E−01 | 0.87 | 4.48E−57 | 0.28 | 3.82E−02 |
| Pomc | 9.15E−01 | 0.03 | 6.62E−01 | −0.18 | 1.32E−01 |
| Cpeb4 | 8.76E−01 | −0.04 | 5.59E−01 | 0.04 | 9.44E−01 |
| Cul2 | 1.00E+00 | −0.11 | 9.35E−02 | 0.02 | 1.00E+00 |
| Hgfac | 1.00E+00 | 0.03 | 2.80E−02 | 0.00 | 1.00E+00 |
| Pofut1 | 1.00E+00 | −0.10 | 6.46E−02 | 0.02 | 1.00E+00 |
| Clasp2 | 1.00E+00 | −0.04 | 4.34E−01 | −0.01 | 1.00E+00 |
| Nme7 | 3.48E−01 | 0.04 | 5.71E−01 | −0.04 | 9.97E−01 |
| Ndst1 | 1.00E+00 | −0.03 | 4.51E−01 | 0.01 | 1.00E+00 |
| Gpr137 | 1.00E+00 | 0.02 | 6.96E−01 | −0.03 | 1.00E+00 |
| Ncoa5 | 1.00E+00 | −0.05 | 4.56E−01 | −0.16 | 3.19E−01 |
| Mapkapk5 | 1.00E+00 | 0.02 | 6.95E−01 | 0.01 | 1.00E+00 |
| Auh | 1.00E+00 | −0.04 | 6.83E−01 | −0.21 | 4.42E−01 |
| Gpx3 | 1.00E+00 | −0.01 | 7.93E−01 | 0.04 | 9.02E−01 |
| Tmem116 | 1.00E+00 | 0.03 | 4.43E−02 | 0.01 | 1.00E+00 |
| Hint1 | 1.00E+00 | −0.05 | 2.86E−01 | 0.03 | 9.94E−01 |
| Mtmr3 | 1.00E+00 | −0.04 | 6.89E−01 | 0.04 | 1.00E+00 |
| Ube2l3 | 1.00E+00 | 0.04 | 5.15E−01 | 0.01 | 1.00E+00 |
| Il23r | 1.00E+00 | −0.06 | 3.06E−02 | 0.01 | 1.00E+00 |
| Asb2 | 1.01E−01 | −0.82 | 1.21E−45 | 0.23 | 4.53E−01 |
| Snx32 | 1.00E+00 | 0.03 | 4.97E−01 | 0.02 | 1.00E+00 |
| Fgfr1op | 1.00E+00 | −0.07 | 2.95E−01 | −0.05 | 1.00E+00 |
| Ccl2 | 1.00E+00 | −0.02 | 6.93E−01 | 0.01 | 1.00E+00 |
| Slc16a10 | 1.00E+00 | −0.04 | 1.79E−01 | −0.06 | 5.37E−01 |
| Batf | 1.00E+00 | 0.19 | 1.01E−04 | −0.08 | 9.31E−01 |
| Trpt1 | 9.96E−01 | −0.01 | 9.76E−01 | 0.02 | 1.00E+00 |
| Jazf1 | 1.00E+00 | −0.02 | 7.34E−01 | −0.05 | 8.91E−01 |
| Tubd1 | 1.00E+00 | −0.03 | 3.98E−01 | −0.02 | 1.00E+00 |
| Rtel1 | 1.00E+00 | 0.05 | 3.81E−01 | −0.08 | 8.67E−01 |
| Srm | 9.15E−01 | −0.06 | 3.16E−01 | 0.06 | 9.41E−01 |
| Thada | 8.76E−01 | −0.23 | 1.60E−07 | −0.03 | 9.94E−01 |
| Tppp | 9.15E−01 | 0.00 | 9.14E−01 | 0.10 | 5.10E−01 |
| Ppp4c | 1.00E+00 | 0.16 | 7.01E−05 | −0.14 | 4.53E−01 |
| Per3 | 1.00E+00 | −0.09 | 1.74E−03 | −0.01 | 1.00E+00 |
| Hormad2 | 1.00E+00 | −0.03 | 9.85E−02 | −0.02 | 5.72E−01 |
| Gm20186 | 1.00E+00 | −0.20 | 8.04E−03 | −0.45 | 5.31E−03 |
| Ccdc122 | 1.00E+00 | 0.01 | 4.72E−01 | −0.03 | 8.72E−01 |
| Fasl | 1.00E+00 | 0.19 | 2.41E−04 | 0.35 | 1.76E−03 |
| Sh2d2a | 5.39E−01 | −0.13 | 6.69E−03 | −0.24 | 5.78E−02 |
| Atp6v0a1 | 1.00E+00 | −0.01 | 7.06E−01 | 0.02 | 9.94E−01 |
| Fosl1 | 1.00E+00 | −0.01 | 5.87E−01 | −0.01 | 9.31E−01 |
| Itgb8 | 1.00E+00 | −0.01 | 6.74E−01 | −0.01 | 9.31E−01 |
| Amz1 | 9.15E−01 | 0.00 | 8.96E−01 | 0.03 | 8.33E−01 |
| Cxcl5 | 1.00E+00 | 0.00 | 6.83E−01 | −0.01 | 9.31E−01 |
| Nkx2-3 | 1.00E+00 | 0.00 | 9.18E−01 | 0.05 | 2.36E−01 |
| Thbs3 | 1.00E+00 | −0.01 | 1.82E−01 | 0.00 | 1.00E+00 |
| Pf4 | 1.00E+00 | −0.02 | 7.34E−01 | −0.01 | 1.00E+00 |
| Ephx4 | 1.00E+00 | 0.00 | 9.01E−01 | 0.00 | 1.00E+00 |
| Osmr | 1.00E+00 | 0.00 | 4.97E−01 | 0.00 | 1.00E+00 |
| Ccl11 | 1.00E+00 | −0.01 | 2.61E−01 | 0.00 | 1.00E+00 |
| Fndc4 | 1.00E+00 | −0.01 | 2.98E−01 | 0.00 | 1.00E+00 |
| Ccl7 | 1.00E+00 | −0.01 | 2.61E−01 | 0.00 | 1.00E+00 |
| Ptrf | 1.00E+00 | 0.01 | 6.89E−01 | 0.02 | 7.31E−01 |
| Mei1 | 1.00E+00 | 0.00 | 4.97E−01 | 0.00 | 1.00E+00 |
| Lamb1 | 1.00E+00 | 0.00 | 4.20E−01 | 0.00 | 1.00E+00 |

| Gene | logFC_LPL_TissueDE | FDRLPL_TissueDE | LPL_Geno_logFc | LPLGeno_FDR |
|---|---|---|---|---|
| Gpr18 | 0.96 | 1.73E−191 | −0.43 | 2.85E−23 |
| Gzmb | 1.73 | 0.00E+00 | −0.07 | 3.72E−01 |
| Itgb1 | −2.90 | 0.00E+00 | −0.02 | 8.68E−01 |
| Ccr3 | −0.01 | 1.50E−01 | 0.01 | 3.64E−01 |
| Gzma | 0.25 | 3.52E−05 | −1.55 | 6.24E−128 |
| Il22 | 2.34 | 1.69E−284 | 0.09 | 5.63E−01 |
| Zfp36l2 | 1.28 | 0.00E+00 | −0.77 | 1.26E−128 |
| Zbtb38 | −0.02 | 7.35E−01 | −0.21 | 4.35E−07 |
| Cd74 | 0.47 | 2.30E−23 | 0.35 | 1.00E−07 |
| Ccr5 | 1.14 | 4.43E−273 | −0.19 | 4.51E−06 |
| Cd160 | 0.54 | 3.27E−37 | −1.38 | 3.92E−160 |
| Map3k8 | 1.57 | 0.00E+00 | −0.05 | 2.66E−01 |
| Tnfsf8 | 2.00 | 0.00E+00 | 0.25 | 1.80E−07 |
| Ifitm1 | −1.25 | 2.03E−53 | 0.27 | 7.06E−02 |
| Nfkbiz | 2.36 | 0.00E+00 | 0.24 | 1.85E−13 |
| Fosl2 | 2.34 | 0.00E+00 | 0.42 | 1.93E−30 |
| Crem | 3.18 | 0.00E+00 | 0.87 | 1.52E−146 |
| Ccdc85b | −0.08 | 3.66E−03 | −0.31 | 1.45E−22 |
| Fos | 1.94 | 0.00E+00 | 0.90 | 3.74E−58 |
| Gpr183 | 1.35 | 0.00E+00 | 0.03 | 5.64E−01 |
| S100a4 | −1.82 | 0.00E+00 | −0.71 | 9.86E−65 |
| 1110008f13rik | −0.29 | 3.34E−38 | −0.02 | 7.17E−01 |
| Lsp1 | −0.86 | 0.00E+00 | −0.41 | 5.73E−94 |
| Litaf | 1.61 | 0.00E+00 | 0.31 | 5.15E−14 |
| Cd7 | −0.36 | 9.57E−25 | −1.40 | 5.09E−150 |
| Dusp2 | −0.23 | 8.06E−13 | −0.42 | 2.72E−20 |
| Plac8 | 1.58 | 0.00E+00 | −0.29 | 8.95E−15 |
| H1f0 | 0.30 | 2.02E−17 | −0.75 | 1.19E−64 |
| S1pr1 | −0.73 | 1.05E−149 | −0.42 | 4.66E−22 |
| Ncf4 | −0.29 | 2.67E−26 | −0.29 | 6.15E−15 |
| Smim3 | 1.46 | 0.00E+00 | 0.20 | 2.60E−06 |
| Tesc | 0.47 | 2.25E−70 | 0.09 | 1.66E−02 |
| Rbms1 | −0.42 | 3.83E−72 | −0.18 | 1.11E−07 |
| Lpxn | 0.97 | 0.00E+00 | −0.04 | 2.29E−01 |
| Tnfrsf9 | 1.37 | 6.99E−304 | 0.50 | 1.36E−26 |
| Pmm1 | −0.02 | 5.25E−01 | −0.15 | 2.31E−04 |
| Tob2 | 1.32 | 0.00E+00 | 0.38 | 7.86E−22 |
| Ifng | 1.69 | 0.00E+00 | 0.45 | 4.62E−16 |
| Cd226 | −0.30 | 8.07E−26 | −0.23 | 6.66E−08 |
| Ctsw | 0.32 | 2.47E−57 | −0.35 | 3.78E−45 |
| Itga4 | −0.39 | 1.20E−42 | −0.44 | 9.68E−35 |
| Cd28 | 1.08 | 0.00E+00 | 0.11 | 1.52E−06 |
| Klf2 | −1.77 | 0.00E+00 | −0.71 | 1.06E−46 |
| Ly6c2 | −2.35 | 0.00E+00 | −0.04 | 8.54E−01 |
| Ccr9 | 0.97 | 9.81E−132 | −0.93 | 5.85E−62 |
| Ets1 | 0.87 | 0.00E+00 | 0.03 | 1.65E−01 |
| Il7r | −0.06 | 6.14E−02 | −0.48 | 7.56E−56 |
| Ptpn22 | 1.18 | 0.00E+00 | −0.14 | 5.52E−08 |
| Gm12840 | 2.19 | 0.00E+00 | −0.09 | 5.83E−02 |
| Zfp36l1 | 1.17 | 0.00E+00 | −0.19 | 8.62E−07 |
| Atxn2l | 0.07 | 2.08E−02 | 0.01 | 8.40E−01 |
| Pde4b | 2.14 | 0.00E+00 | −0.02 | 6.98E−01 |
| Selp | 0.00 | 2.17E−01 | 0.00 | 9.89E−01 |
| Anxa6 | −0.83 | 0.00E+00 | −0.20 | 3.31E−15 |
| Peli1 | 0.92 | 0.00E+00 | 0.12 | 2.03E−05 |
| Odc1 | 3.27 | 0.00E+00 | 0.72 | 2.42E−135 |
| Pglyrp1 | −1.58 | 0.00E+00 | −0.70 | 2.94E−61 |
| Pim3 | 1.12 | 2.33E−266 | 0.33 | 3.82E−14 |
| Pim1 | 2.65 | 0.00E+00 | 0.14 | 3.43E−07 |
| Mt1 | 0.65 | 1.10E−60 | −0.23 | 8.28E−05 |
| Stat3 | 1.08 | 0.00E+00 | 0.50 | 5.92E−61 |
| Stat5a | 0.47 | 7.41E−57 | 0.24 | 1.92E−09 |
| Vmp1 | 0.51 | 1.96E−93 | −0.22 | 2.35E−11 |
| Ier2 | 0.43 | 6.68E−88 | −0.02 | 5.61E−01 |
| Tnip1 | 0.77 | 2.23E−173 | 0.22 | 1.10E−10 |
| Timp1 | 0.00 | 5.92E−02 | 0.00 | 4.94E−01 |
| Amdhd1 | 0.00 | 7.11E−01 | 0.00 | 8.98E−01 |
| Rora | 1.63 | 0.00E+00 | 0.12 | 3.53E−05 |
| Gm5111 | 0.01 | 7.54E−02 | −0.02 | 6.99E−03 |
| Fermt3 | −0.46 | 4.11E−84 | −0.36 | 5.96E−27 |
| Dap | −0.33 | 2.26E−36 | −0.28 | 2.10E−15 |
| Hist1h1c | 1.90 | 0.00E+00 | −0.48 | 4.45E−25 |
| Cfl1 | −0.48 | 0.00E+00 | −0.14 | 3.20E−22 |
| Ipmk | 0.92 | 4.46E−219 | 0.27 | 5.98E−10 |
| Klrd1 | −1.07 | 1.81E−199 | −0.34 | 2.65E−12 |
| Gfi1 | −0.01 | 7.95E−01 | −0.09 | 7.38E−02 |
| S100a6 | −1.05 | 0.00E+00 | −0.50 | 2.02E−69 |
| Rorc | 0.02 | 2.72E−01 | −0.07 | 3.21E−03 |
| Stat4 | 0.87 | 7.48E−245 | 0.35 | 3.64E−28 |
| Fdps | −0.05 | 1.34E−01 | −0.15 | 1.10E−03 |
| Gpr65 | 0.86 | 4.47E−166 | 0.17 | 1.39E−04 |
| Rapgef6 | 0.41 | 8.06E−51 | −0.10 | 1.05E−02 |
| Tmem160 | −0.11 | 6.65E−08 | −0.20 | 8.36E−13 |
| Rasgrp1 | 0.84 | 1.94E−202 | 0.12 | 2.14E−03 |
| Pnkd | −0.24 | 4.02E−19 | −0.21 | 3.09E−08 |
| Ccnd3 | −1.12 | 0.00E+00 | 0.02 | 7.93E−01 |
| Panx1 | −0.17 | 1.23E−09 | 0.01 | 8.01E−01 |
| Thap11 | −0.29 | 2.70E−26 | −0.13 | 6.17E−04 |
| Lysmd2 | 0.09 | 1.88E−03 | −0.25 | 2.67E−09 |
| Ubac2 | 0.29 | 3.46E−29 | −0.17 | 2.34E−06 |
| Lgals3 | −0.30 | 1.46E−24 | 0.05 | 3.04E−01 |
| Cyb5r4 | −0.50 | 2.63E−95 | −0.29 | 9.44E−17 |
| Upf3a | 0.02 | 5.65E−01 | −0.34 | 8.61E−21 |
| Gm10076 | 0.20 | 5.88E−17 | −0.69 | 1.02E−153 |
| Ccdc69 | −0.49 | 4.22E−76 | −0.18 | 1.53E−07 |
| Ccl5 | 0.23 | 5.28E−10 | −0.50 | 1.70E−33 |
| Gramd3 | 1.92 | 0.00E+00 | −0.22 | 1.66E−13 |
| Errfi1 | 0.75 | 2.26E−126 | 0.43 | 1.84E−24 |
| Itgae | 0.83 | 3.08E−116 | −1.03 | 1.01E−109 |
| Il2ra | 0.74 | 2.94E−134 | 0.22 | 9.64E−07 |
| Gpx4 | 0.72 | 0.00E+00 | 0.22 | 6.61E−24 |
| Ndfip1 | 0.72 | 8.87E−237 | 0.19 | 8.28E−12 |
| Syt11 | −0.61 | 4.65E−99 | −0.11 | 1.72E−02 |
| Dph5 | −0.39 | 2.02E−42 | 0.06 | 2.10E−01 |
| Slc39a8 | 0.08 | 6.23E−05 | 0.01 | 8.96E−01 |
| Rnf187 | −0.15 | 1.19E−11 | −0.05 | 2.23E−01 |
| Fam53b | −0.83 | 4.51E−193 | −0.14 | 1.68E−03 |
| Prdx5 | 0.37 | 1.56E−69 | 0.12 | 4.74E−05 |
| Ccrl2 | 0.67 | 3.95E−127 | 0.14 | 2.06E−02 |
| Ep300 | −0.06 | 4.74E−02 | 0.00 | 9.89E−01 |
| Hdac7 | −0.21 | 2.97E−15 | −0.10 | 1.22E−02 |
| Cd6 | 0.21 | 1.47E−23 | −0.04 | 3.00E−01 |
| Traf3ip2 | −0.30 | 2.68E−31 | −0.09 | 2.46E−02 |
| Gnb2 | −0.28 | 9.03E−64 | −0.25 | 5.08E−28 |
| Tet2 | 0.66 | 2.79E−130 | 0.14 | 6.32E−04 |
| Fam189b | −0.31 | 1.60E−32 | −0.07 | 1.12E−01 |
| Skap2 | −0.02 | 4.09E−01 | −0.13 | 1.54E−03 |
| Lgals1 | −1.15 | 0.00E+00 | −0.17 | 1.87E−14 |
| Socs1 | 0.64 | 5.14E−93 | 0.46 | 3.55E−32 |
| Itgav | 0.64 | 5.62E−112 | 0.24 | 8.61E−09 |
| Dnajb4 | 0.63 | 7.45E−98 | 0.38 | 1.46E−19 |
| Cdkn1a | 2.23 | 0.00E+00 | 0.77 | 1.83E−88 |
| Arhgef2 | −0.77 | 1.60E−174 | −0.23 | 6.78E−10 |
| Dnmt3a | −0.20 | 1.16E−14 | −0.11 | 3.41E−03 |
| Jund | 2.11 | 0.00E+00 | 0.61 | 1.37E−184 |
| Scamp3 | −0.26 | 1.01E−30 | −0.23 | 3.47E−13 |
| Tmem30a | −0.08 | 2.09E−03 | −0.35 | 1.16E−26 |
| Ash1l | −0.29 | 2.45E−23 | −0.05 | 2.62E−01 |
| Lsm14a | −0.33 | 4.06E−43 | −0.26 | 1.10E−14 |
| Slain2 | −0.04 | 1.61E−01 | −0.06 | 2.29E−01 |
| Muc1 | 0.38 | 1.56E−88 | 0.16 | 8.62E−06 |
| Adamts14 | 0.00 | 4.18E−01 | 0.00 | 3.99E−01 |
| Ptk2b | 0.09 | 1.97E−03 | 0.01 | 9.09E−01 |
| Stk11 | 0.59 | 2.94E−110 | 0.28 | 3.12E−17 |
| Ccr2 | 0.23 | 3.04E−18 | 0.03 | 4.65E−01 |
| Itgal | −0.19 | 2.04E−14 | −0.11 | 6.79E−03 |
| Elf1 | 0.57 | 2.94E−128 | −0.05 | 1.85E−01 |
| Ptprc | 0.02 | 2.51E−01 | −0.16 | 3.89E−15 |
| Rbx1 | −0.46 | 3.93E−187 | −0.14 | 4.63E−09 |
| Traf3ip3 | −0.85 | 2.40E−253 | −0.45 | 4.63E−34 |
| Ccdc88b | 0.06 | 3.10E−02 | −0.08 | 9.71E−02 |
| Tsen34 | −0.38 | 5.90E−55 | −0.05 | 1.20E−01 |
| Ay036118 | 0.21 | 4.92E−12 | −0.25 | 9.09E−11 |
| Il24 | | | | |
| H2-q10 | 0.06 | 5.93E−02 | 0.34 | 4.29E−19 |
| Scand1 | 0.38 | 4.88E−74 | −0.12 | 3.10E−05 |
| Tab2 | 0.26 | 1.01E−20 | −0.01 | 8.65E−01 |
| Nfkb1 | 0.53 | 7.48E−97 | 0.20 | 7.58E−10 |
| Rusc1 | 0.11 | 1.24E−04 | −0.05 | 3.89E−01 |
| Esyt1 | −0.90 | 6.87E−301 | −0.24 | 3.30E−11 |
| Commd7 | −0.23 | 7.48E−19 | −0.08 | 4.85E−02 |
| Rfng | −0.05 | 9.05E−02 | −0.14 | 2.09E−05 |
| Rpl27-ps3 | −0.04 | 8.61E−02 | −0.40 | 1.29E−45 |
| Gigyf1 | −0.02 | 7.64E−01 | −0.03 | 5.00E−01 |
| Rgcc | 1.58 | 0.00E+00 | 1.13 | 9.68E−131 |
| Rpl3 | 0.05 | 1.55E−11 | −0.19 | 6.04E−99 |
| Mrpl20 | −0.25 | 3.76E−28 | −0.16 | 1.68E−06 |
| Gm2a | 0.18 | 5.94E−14 | 0.01 | 7.98E−01 |
| Mus81 | −0.25 | 1.10E−24 | −0.13 | 2.72E−05 |
| Lpp | −0.08 | 8.01E−03 | −0.05 | 3.01E−01 |
| Chp1 | 0.14 | 4.48E−07 | −0.03 | 5.50E−01 |
| Mob4 | 0.24 | 4.38E−22 | 0.00 | 9.70E−01 |
| Lamp1 | −0.09 | 1.99E−03 | −0.08 | 6.29E−02 |
| Phf5a | −0.12 | 1.13E−06 | −0.14 | 4.68E−05 |
| Fnip1 | 0.25 | 4.48E−27 | 0.08 | 5.52E−02 |
| Calm3 | −0.41 | 2.42E−100 | −0.07 | 2.01E−02 |
| Lrp10 | −0.42 | 1.30E−69 | −0.20 | 1.15E−08 |
| Stat5b | 0.45 | 8.98E−58 | 0.03 | 5.23E−01 |
| Bach2 | 0.00 | 8.46E−01 | −0.02 | 4.95E−01 |
| Prkab1 | −0.35 | 6.30E−38 | 0.08 | 4.65E−02 |
| Hvcn1 | 0.04 | 1.62E−01 | −0.01 | 8.83E−01 |
| Tmem150a | 0.00 | 4.94E−01 | 0.00 | 9.89E−01 |
| Nfkbia | 2.07 | 0.00E+00 | 0.12 | 8.93E−05 |
| Ifitm3 | −0.83 | 1.03E−104 | −0.28 | 5.76E−08 |
| Egfp_ires_il23r | | | | |
| Bad | −0.41 | 1.49E−50 | −0.13 | 1.83E−03 |
| Zbtb46 | 0.21 | 2.32E−49 | −0.02 | 7.54E−01 |
| Cacna1s | 0.10 | 1.50E−12 | 0.07 | 5.13E−02 |
| Vamp3 | 0.11 | 1.11E−04 | 0.00 | 9.72E−01 |
| Fyn | −0.07 | 4.20E−03 | −0.03 | 4.63E−01 |
| Stx6 | 0.29 | 1.07E−25 | 0.10 | 1.24E−02 |
| Ikzf3 | 0.41 | 2.25E−36 | 0.33 | 4.87E−15 |
| Mgat2 | −0.26 | 4.37E−28 | 0.05 | 3.30E−01 |
| Serpinb9 | 1.70 | 0.00E+00 | 0.21 | 1.47E−07 |
| Dennd1b | −0.03 | 3.51E−01 | −0.04 | 3.92E−01 |
| Bloc1s4 | 0.27 | 2.06E−22 | 0.04 | 3.99E−01 |
| Anxa1 | −1.54 | 0.00E+00 | 0.09 | 2.17E−01 |
| Smad3 | 0.10 | 2.25E−04 | −0.01 | 8.89E−01 |
| Tmem258 | −0.20 | 4.26E−30 | −0.08 | 3.54E−03 |
| Lmna | 1.99 | 0.00E+00 | 1.14 | 3.52E−154 |
| Sp140 | −0.08 | 9.86E−03 | −0.09 | 6.70E−02 |
| Actn2 | 1.25 | 0.00E+00 | −0.55 | 1.65E−44 |
| Acbd6 | 0.22 | 5.41E−17 | −0.04 | 4.00E−01 |
| Ahnak | 0.16 | 3.21E−12 | 0.09 | 5.88E−03 |
| Arpc2 | −0.07 | 4.13E−12 | −0.06 | 2.31E−05 |
| Erp29 | −0.16 | 2.63E−10 | −0.03 | 5.61E−01 |
| Nhp2l1 | 0.37 | 1.56E−79 | 0.24 | 1.76E−23 |
| Ctbp1 | −0.21 | 4.08E−23 | −0.32 | 5.84E−28 |
| Gba | −0.08 | 5.03E−03 | −0.13 | 8.46E−04 |
| Lztfl1 | 1.24 | 0.00E+00 | −0.30 | 3.32E−13 |
| Ptpn2 | 0.35 | 1.25E−39 | 0.04 | 4.25E−01 |
| Usp36 | 0.35 | 5.76E−37 | 0.05 | 3.77E−01 |
| Grb7 | 0.34 | 2.65E−72 | 0.17 | 1.68E−05 |
| Fndc3a | 0.34 | 2.71E−37 | 0.09 | 4.78E−02 |
| Mt2 | 0.21 | 1.44E−13 | −0.16 | 3.94E−05 |
| Dctn4 | 0.33 | 2.12E−33 | −0.10 | 2.80E−02 |
| Nusap1 | −0.73 | 1.52E−56 | −0.33 | 1.32E−09 |
| Rbm22 | 0.03 | 3.51E−01 | −0.07 | 1.15E−01 |
| Crip1 | −0.76 | 0.00E+00 | 0.11 | 1.42E−04 |
| Anp32b | −0.48 | 1.60E−118 | −0.19 | 2.26E−10 |
| Aurkaip1 | −0.26 | 5.78E−38 | −0.05 | 1.47E−01 |
| Ripk2 | 0.36 | 9.55E−40 | 0.05 | 3.16E−01 |
| Nubp1 | −0.12 | 1.43E−05 | −0.37 | 1.05E−25 |
| Camta1 | 0.31 | 1.48E−28 | −0.19 | 1.30E−06 |
| Ube2j2 | 0.30 | 1.69E−44 | 0.00 | 9.68E−01 |
| Kif3b | −0.10 | 1.34E−06 | −0.03 | 2.61E−01 |
| Brap | 0.30 | 4.45E−28 | 0.01 | 8.00E−01 |
| Gm8096 | | | | |
| Ermn | 1.01 | 4.90E−162 | −0.05 | 6.77E−01 |
| Plagl2 | −0.01 | 8.42E−01 | −0.03 | 4.69E−01 |
| Plek | −0.25 | 1.29E−15 | 0.20 | 5.12E−05 |
| D030056l22rik | 0.23 | 5.67E−14 | 0.18 | 5.19E−05 |
| Ccr1 | 0.28 | 7.44E−31 | 0.20 | 6.92E−06 |
| Krtcap2 | −0.07 | 9.75E−06 | 0.14 | 1.66E−11 |
| Sh2b1 | 0.28 | 5.19E−22 | −0.14 | 9.77E−04 |
| Abi3 | 0.97 | 2.99E−212 | −0.09 | 6.99E−02 |
| Ccnl2 | 0.27 | 6.75E−30 | −0.12 | 4.01E−04 |
| Kbtbd7 | 0.01 | 7.26E−01 | 0.00 | 9.31E−01 |
| Csf2 | 0.27 | 9.13E−25 | 0.10 | 3.81E−01 |
| Pycard | −1.19 | 0.00E+00 | −0.63 | 4.09E−86 |
| Dock7 | 0.00 | 4.69E−01 | 0.00 | 9.89E−01 |
| Herpud1 | 1.16 | 0.00E+00 | 0.13 | 1.68E−04 |
| Arf6 | 0.39 | 1.96E−101 | 0.04 | 1.73E−01 |
| Zyx | −1.05 | 0.00E+00 | −0.52 | 4.55E−41 |
| Zfp91 | 0.25 | 9.43E−19 | 0.01 | 9.89E−01 |
| Fam171b | 0.00 | 9.50E−01 | −0.02 | 2.29E−01 |
| Maml2 | −0.07 | 1.11E−02 | −0.04 | 3.99E−01 |
| Ifngr2 | 0.24 | 7.48E−36 | 0.07 | 2.88E−02 |
| Bhlhe40 | 2.48 | 0.00E+00 | 0.02 | 4.83E−01 |
| Cisd1 | −0.22 | 1.92E−15 | −0.23 | 3.43E−09 |
| Zfp420 | 0.01 | 4.10E−01 | 0.01 | 6.99E−01 |
| Zc3h12a | 2.18 | 0.00E+00 | 0.27 | 2.84E−16 |
| Plcg2 | 0.01 | 9.88E−01 | −0.05 | 1.75E−02 |
| 2310036o22rik | −0.31 | 3.53E−60 | −0.13 | 6.10E−06 |
| Eif3c | −0.15 | 1.10E−11 | −0.04 | 2.64E−01 |
| Slc15a3 | 0.52 | 3.74E−74 | 0.26 | 8.00E−09 |
| Acap3 | −0.13 | 1.29E−08 | −0.01 | 9.21E−01 |
| Tef | 0.21 | 2.35E−22 | 0.06 | 1.33E−01 |
| Emp3 | −1.21 | 0.00E+00 | −0.34 | 2.78E−39 |
| Ssr2 | −0.40 | 1.17E−65 | −0.20 | 9.50E−09 |
| Usp1 | −0.30 | 5.06E−26 | −0.19 | 2.01E−06 |
| Rpl10-ps3 | −0.35 | 1.51E−57 | −0.40 | 8.76E−41 |
| Plcxd2 | 1.34 | 0.00E+00 | −0.10 | 5.09E−02 |
| Adcy7 | 0.01 | 8.47E−01 | −0.18 | 2.99E−06 |
| Irf1 | 0.18 | 1.70E−09 | 0.26 | 5.53E−11 |
| Aamp | 0.17 | 2.36E−13 | 0.15 | 2.87E−06 |
| Gm8730 | −0.08 | 1.81E−08 | −0.61 | 8.94E−278 |
| Zgpat | −0.03 | 3.39E−01 | −0.17 | 1.52E−05 |
| St13 | 0.17 | 5.97E−12 | 0.22 | 1.00E−12 |
| Cdc42se2 | 0.17 | 2.04E−12 | −0.09 | 1.19E−02 |
| Anxa2 | −0.54 | 2.39E−116 | 0.15 | 1.29E−05 |
| Cpsf3l | −0.45 | 9.24E−61 | −0.16 | 9.05E−05 |
| Gm2000 | −0.37 | 7.22E−67 | −0.47 | 4.20E−62 |
| Trim46 | 0.00 | 9.99E−01 | −0.01 | 7.82E−01 |
| Dusp16 | 0.15 | 3.73E−34 | 0.04 | 2.24E−01 |
| Zmat5 | −0.35 | 2.87E−41 | −0.15 | 1.08E−04 |
| Ptprk | −0.03 | 1.26E−01 | −0.15 | 3.37E−06 |
| Zdhhc7 | −0.08 | 3.65E−03 | 0.00 | 9.65E−01 |
| Il2 | 0.14 | 1.06E−05 | 0.06 | 2.32E−01 |
| Msto1 | −0.13 | 2.19E−07 | −0.14 | 5.82E−06 |
| Il10 | 0.14 | 2.17E−05 | 0.27 | 1.69E−08 |
| Sbno2 | 0.14 | 2.71E−19 | 0.02 | 6.45E−01 |
| Snx17 | −0.21 | 2.10E−16 | −0.14 | 1.67E−04 |
| Kif11 | −0.28 | 2.26E−31 | −0.14 | 3.85E−07 |
| Ndfip2 | 0.08 | 2.32E−03 | −0.08 | 3.29E−02 |
| Card9 | −0.01 | 1.41E−01 | 0.00 | 8.89E−01 |
| Lsm4 | −0.35 | 5.60E−77 | −0.25 | 6.16E−21 |
| Ormdl3 | 0.13 | 1.24E−05 | 0.16 | 7.45E−05 |
| S100a10 | −0.84 | 0.00E+00 | −0.03 | 1.80E−01 |
| Notch2 | −0.03 | 5.11E−01 | −0.13 | 3.92E−03 |
| Senp7 | −0.20 | 5.37E−13 | −0.12 | 1.26E−03 |
| Cd48 | −1.55 | 0.00E+00 | −0.52 | 2.68E−69 |
| Ssbp4 | 0.26 | 2.33E−29 | −0.15 | 2.08E−06 |
| Cul1 | 0.12 | 3.32E−05 | −0.01 | 8.54E−01 |
| Mettl10 | −0.20 | 5.48E−13 | −0.12 | 3.84E−03 |
| Rabep2 | −0.27 | 7.52E−23 | −0.11 | 2.82E−03 |
| Pkig | 0.11 | 4.31E−08 | 0.06 | 1.22E−01 |
| Desi1 | 0.11 | 3.42E−04 | −0.11 | 9.85E−03 |
| Furin | 3.23 | 0.00E+00 | 0.30 | 1.88E−17 |
| Tcof1 | −0.40 | 4.30E−48 | −0.11 | 1.01E−02 |
| Glipr2 | −1.36 | 0.00E+00 | −0.43 | 3.68E−38 |
| Tgfbr3 | 0.11 | 1.03E−08 | 0.00 | 9.89E−01 |
| Tyk2 | −0.09 | 6.73E−04 | −0.10 | 4.23E−03 |
| Kif21b | −0.27 | 1.72E−21 | −0.10 | 1.08E−02 |
| Tufm | −0.39 | 1.54E−46 | −0.10 | 1.12E−02 |
| Zmiz1 | −0.23 | 2.23E−15 | −0.10 | 1.92E−02 |
| Il21 | 0.10 | 5.13E−06 | 0.07 | 1.23E−01 |
| Ttpal | 0.10 | 1.87E−04 | −0.10 | 1.11E−02 |
| Rps6kb1 | 0.10 | 5.80E−04 | 0.14 | 2.52E−04 |
| Camk2g | −0.16 | 4.70E−08 | −0.10 | 2.24E−02 |
| Tom1 | −0.06 | 1.33E−02 | −0.10 | 4.27E−03 |
| Actr1a | −0.19 | 3.12E−12 | −0.10 | 2.29E−02 |
| Rgs14 | −0.39 | 3.10E−46 | −0.10 | 2.57E−02 |
| Ublcp1 | −0.15 | 3.40E−09 | −0.10 | 6.49E−03 |
| Otud3 | 0.09 | 1.54E−07 | 0.00 | 9.81E−01 |
| Tmem180 | 0.09 | 1.18E−05 | 0.01 | 8.13E−01 |
| Ergic1 | 0.09 | 2.69E−04 | 0.05 | 3.21E−01 |
| Aa467197 | −0.49 | 3.65E−58 | −0.16 | 3.18E−04 |
| Tdp2 | 0.25 | 2.02E−16 | 0.08 | 1.26E−01 |
| Rac1 | −0.03 | 7.10E−02 | −0.06 | 9.85E−03 |
| Phlda3 | −0.24 | 1.02E−25 | 0.04 | 3.99E−01 |
| Trmt112 | −0.22 | 2.35E−26 | −0.09 | 7.80E−03 |
| Ccny | 0.07 | 1.27E−02 | −0.08 | 5.36E−02 |
| Slc43a3 | −0.14 | 1.56E−10 | −0.08 | 1.18E−03 |
| Ezh2 | −0.52 | 5.20E−63 | −0.08 | 4.97E−02 |
| Rps14 | 0.08 | 4.48E−27 | 0.00 | 9.98E−01 |
| Spred2 | 0.08 | 2.35E−05 | 0.06 | 5.38E−02 |
| Rn7s2 | | | | |
| Rn7s1 | | | | |
| Rpap2 | −0.10 | 4.04E−05 | −0.08 | 1.47E−02 |
| Cit | −0.45 | 5.44E−71 | −0.08 | 5.23E−03 |
| Ascc2 | −0.11 | 3.83E−06 | −0.08 | 2.40E−02 |
| Ksr1 | −0.08 | 1.46E−04 | −0.08 | 3.19E−03 |
| Edem2 | −0.33 | 1.05E−32 | −0.08 | 7.89E−02 |
| Blzf1 | 0.08 | 2.57E−03 | 0.08 | 4.28E−02 |
| Ppp5c | −0.21 | 1.24E−13 | −0.08 | 8.53E−02 |
| Smurf1 | 0.08 | 1.11E−04 | −0.04 | 2.94E−01 |
| Nop9 | −0.04 | 2.17E−01 | −0.08 | 8.95E−02 |
| Slc30a7 | −0.27 | 1.11E−24 | −0.08 | 4.46E−02 |
| Tnfrsf8 | 0.01 | 2.34E−01 | 0.01 | 7.17E−01 |
| Adcy3 | −0.27 | 4.90E−33 | −0.08 | 1.04E−02 |
| Pdcd1 | −0.14 | 5.58E−05 | 0.38 | 1.56E−12 |
| Fen1 | −0.29 | 1.27E−22 | −0.07 | 8.71E−02 |
| Themis | 0.07 | 1.93E−02 | −0.07 | 7.05E−02 |
| Nudt22 | −0.26 | 9.63E−24 | −0.07 | 4.38E−02 |
| Cyth1 | 0.07 | 1.27E−02 | −0.01 | 9.32E−01 |
| Tmbim1 | 0.07 | 5.46E04 | 0.06 | 9.22E−02 |
| Ndufaf1 | −0.21 | 3.92E−16 | −0.07 | 6.18E−02 |
| Klrc1 | 0.33 | 8.00E−22 | 0.15 | 2.72E−03 |
| Pomc | 0.07 | 6.94E−06 | −0.03 | 2.65E−01 |
| Cpeb4 | 0.07 | 8.20E−05 | 0.04 | 2.26E−01 |
| Cul2 | 0.07 | 1.47E−02 | 0.06 | 2.27E−01 |
| Hgfac | −0.01 | 2.52E−02 | 0.00 | 6.98E−01 |
| Pofut1 | 0.01 | 6.89E−01 | −0.07 | 5.41E−02 |
| Clasp2 | −0.02 | 3.61E−01 | −0.06 | 4.22E−02 |
| Nme7 | −0.15 | 1.54E−13 | −0.06 | 1.67E−02 |
| Ndst1 | 0.06 | 3.15E−07 | −0.02 | 7.44E−01 |
| Gpr137 | 0.06 | 3.32E−04 | 0.00 | 9.89E−01 |
| Ncoa5 | −0.18 | 2.59E−15 | −0.06 | 8.44E−02 |
| Mapkapk5 | −0.01 | 5.29E−01 | −0.06 | 3.66E−02 |
| Auh | 0.05 | 8.17E−02 | −0.02 | 7.13E−01 |
| Gpx3 | 0.05 | 1.20E−10 | 0.01 | 7.82E−01 |
| Tmem116 | 0.00 | 4.95E−01 | 0.00 | 9.13E−01 |
| Hint1 | −0.21 | 1.37E−47 | −0.05 | 2.25E−02 |
| Mtmr3 | 0.05 | 5.19E−02 | 0.03 | 4.45E−01 |
| Ube2l3 | −0.05 | 1.87E−02 | −0.05 | 9.69E−02 |
| Il23r | 0.05 | 2.13E−05 | 0.01 | 3.99E−01 |
| Asb2 | 1.12 | 3.45E−261 | 0.22 | 4.47E−08 |
| Snx32 | −0.14 | 2.89E−16 | −0.05 | 2.48E−02 |
| Fgfr1op | 0.04 | 8.22E−02 | −0.01 | 9.15E−01 |
| Ccl2 | 0.04 | 4.18E−11 | 0.02 | 4.55E−01 |
| Slc16a10 | 0.04 | 4.30E−05 | −0.02 | 3.99E−01 |
| Batf | 0.76 | 4.63E−195 | −0.09 | 2.50E−02 |
| Trpt1 | 0.04 | 7.43E−03 | 0.01 | 8.02E−01 |
| Jazf1 | 0.04 | 1.18E−02 | −0.02 | 3.32E−01 |
| Tubd1 | −0.14 | 2.33E−19 | −0.04 | 2.75E−02 |
| Rtel1 | −0.08 | 8.41E−06 | −0.04 | 3.22E−02 |
| Srm | −0.46 | 6.71E−57 | −0.08 | 5.19E−02 |
| Thada | 0.03 | 4.55E−02 | 0.04 | 3.99E−01 |
| Tppp | 0.03 | 3.23E−05 | 0.05 | 8.78E−04 |
| Ppp4c | −0.27 | 3.36E−47 | −0.24 | 1.20E−20 |
| Per3 | 0.03 | 5.93E−02 | 0.00 | 8.86E−01 |
| Hormad2 | 0.03 | 1.67E−04 | −0.01 | 6.21E−01 |
| Gm20186 | 1.13 | 8.50E−290 | −0.13 | 8.29E−03 |
| Ccdc122 | 0.00 | 8.24E−01 | −0.02 | 1.13E−02 |
| Fasl | 1.65 | 0.00E+00 | 0.26 | 4.15E−15 |
| Sh2d2a | 1.54 | 0.00E+00 | −0.08 | 8.11E−03 |
| Atp6v0a1 | 0.02 | 1.45E−02 | 0.01 | 9.38E−01 |
| Fosl1 | 0.01 | 7.04E−05 | 0.01 | 2.56E−01 |
| Itgb8 | 0.01 | 9.57E−02 | 0.01 | 4.42E−01 |
| Amz1 | 0.01 | 2.39E−02 | 0.01 | 2.94E−01 |
| Cxcl5 | 0.01 | 2.03E−04 | 0.00 | 3.64E−01 |
| Nkx2-3 | 0.01 | 5.48E−04 | 0.01 | 1.81E−01 |
| Thbs3 | 0.01 | 4.77E−03 | 0.02 | 4.17E−03 |
| Pf4 | 0.01 | 7.65E−02 | 0.00 | 8.61E−01 |
| Ephx4 | 0.00 | 8.43E−01 | −0.01 | 9.78E−02 |
| Osmr | 0.00 | 2.42E−01 | −0.01 | 3.74E−02 |
| Ccl11 | 0.01 | 3.59E−03 | 0.01 | 1.30E−01 |
| Fndc4 | 0.01 | 4.96E−03 | 0.01 | 4.45E−01 |
| Ccl7 | 0.01 | 2.68E−03 | 0.01 | 2.94E−01 |
| Ptrf | 0.01 | 1.09E−02 | 0.01 | 5.60E−01 |
| Mei1 | 0.00 | 4.07E−02 | 0.00 | 7.56E−01 |
| Lamb1 | 0.00 | 1.95E−02 | 0.00 | 7.59E−01 |

Headings description (All logFC are log2 based):
Final Score: Total score used to rank genes
In-Vivo Cluster: Combined score for in-vivo cluster-specific results
In-Vivo Tissue: Combined score for in-vivo tissue-specific results
In-Vitro Summary: Combined score for in-vitro results
GWAS?: Whether or not the gene is associated with an IBD GWAS loci
LPL-9_logFC: logFC of the gene's expression in the LPL-9 cluster vs LPL remainder comparison
LPL-9_FDR: FDR value for the gene in the LPL-9 cluster vs LPL remainder comparison
LPL-9_Geno_logFC: logFC of the gene's expression in the LPL-9 within-cluster Wild Type vs Knockout comparison
LPL-9_Geno_FDR: FDR value for the gene in the LPL-9 within-cluster Wild Type vs Knockout comparison
LPL-2_logFC: logFC of the gene's expression in the LPL-2 cluster vs LPL remainder comparison
LPL-2_FDR: FDR value for the gene in the LPL-2 cluster vs LPL remainder comparison
LPL-2_Geno_logFC: logFC of the gene's expression in the LPL-2 within-cluster Wild Type vs Knockout comparison
LPL-2_Geno_FDR: FDR value for the gene in the LPL-2 within-cluster Wild Type vs Knockout comparison
logFC_LPL_TissueDE: logFC of the gene's expression in the LPL vs. Spleen comparison
FDR_LPL_TissueDE: FDR value for the gene in the LPL vs. Spleen comparison
LPL_Geno_logFC: logFC of the gene's expression in the within-LPL Wild Type vs Knockout comparison
LPL_Geno_FDR: FDR value for the gene in the within-LPL Wild Type vs Knockout comparison

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed:

1. A method of treating an autoimmune disease caused by pathogenic Th1 cells comprising administering one or more agents capable of inhibiting the expression, activity and/or function of one or more genes selected from the group consisting of CD160, GPR18, GZMB, ITGB1, CCR3, GZMA, IL22, ZFP36L2, ZBTB38, CD74, CCR5, MAP3K8, TNFSF8, IFITM1, NFKBIZ, FOSL2, CREM, CCDC85B, FOS, GPR183, S100A4, 1110008F13RIK, LSP1, LITAF, CD7, DUSP2, PLAC8, H1F0, S1PR1, NCF4, SMIM3, TESC, RBMS1, LPXN, TNFRSF9, PMM1, TOB2, IFNG, CD226, and CTSW.

2. The method of claim 1, wherein the one or more agents inhibit the expression, activity and/or function of CD160.

3. The method of claim 1, wherein the one or more agents inhibit the expression, activity and/or function of GPR18.

4. The method of any one of claims 1 to 3, wherein the one or more agents comprises an antibody, antibody fragment, intrabody, small molecule, small molecule degrader, antibody-like protein scaffold, aptamer, polypeptide, genetic modifying agent, or any combination thereof.

5. The method of claim 4, wherein the genetic modifying agent comprises an RNA-guided nuclease system, RNAi system, a zinc finger nuclease, a TALE, or a meganuclease.

6. The method of claim 5, wherein the RNA-guided nuclease system is a CRISPR system or IscB system.

7. The method of claim 6, wherein the CRISPR system comprises a CRISPR-Cas base editing system, a prime editor system, or a CAST system.

8. A method of treating an autoimmune disease caused by pathogenic Th1 cells comprising administering Th1 cells modified to have decreased expression of IL-23R.

9. A method of detecting a Th1 inflammatory response for diagnosis or monitoring of a treatment of a subject suffering from an autoimmune disease caused by colitogenic Th1 cells comprising detecting in a sample obtained from the subject Th1 cells expressing one or more genes selected from the group consisting of:
a. IL23R, CD160, GPR18, GZMB, ITGB1, CCR3, GZMA, IL22, ZFP36L2, ZBTB38, CD74, CCR5, MAP3K8, TNFSF8, IFITM1, NFKBIZ, FOSL2, CREM, CCDC85B, FOS, GPR183, S100A4, 1110008F13RIK, LSP1, LITAF, CD7, DUSP2, PLAC8, H1F0, S1PR1, NCF4, SMIM3, TESC, RBMS1, LPXN, TNFRSF9, PMM1, TOB2, IFNG, CD226, and CTSW, wherein the expression of one or more genes are increased in an inflammatory response; or
b. clusters 2, 9 and 7 in Table 3, wherein the expression of one or more genes or program from cluster 2 and/or 9 are increased in an inflammatory response and the expression of one or more genes or program in cluster 7 are decreased in an inflammatory response.

10. The method of claim 9, wherein the treatment comprises one or more agents capable of inhibiting the expression, activity and/or function of CD160, GPR18, IL-23, or IL-12 and IL-23.

11. The method of claim 9 or 10, wherein the Th1 cells are detected by immunohistochemistry (IHC), fluorescence activated cell sorting (FACS), fluorescently bar-coded oligonucleotide probes, RNA FISH (fluorescent in situ hybridization), RNA-seq, or any combination thereof.

12. The method of claim 11, wherein the Th1 cell expression is inferred from bulk RNA-seq.

13. The method of claim 11, wherein the Th1 cell expression is determined by single cell RNA-seq.

14. The method of any of claims 9 to 13, wherein the sample is obtained by biopsy.

15. The method of any of claims 1 to 14, wherein the autoimmune disease is inflammatory bowel disease (IBD) or type 1 diabetes.

16. A method of obtaining IL-23R⁺ Th1 cells comprising differentiating naïve CD4+ T cells in vitro with IL-12 and IL-21.

17. The method of claim 16, further comprising differentiating with IL-23.

18. A method of treating cancer comprising administering to a subject in need thereof Th1 cells differentiated according to claim 16 or 17.

19. The method of claim 18, wherein the naïve CD4+ T cells are obtained from the subject.

20. A method of screening for drugs capable of shifting pathogenic Th1 cells to non-pathogenic Th1 cells comprising:
a. treating Th1 cells obtained according to claim 16 or 17 with a drug candidate;
b. detecting expression of one or more genes or program selected from cluster 2, 9 or 7 in Table 3; and
c. identifying the drug,
wherein the expression of one or more genes or program from cluster 2 or 9 decrease, and/or the expression of one or more genes or program in cluster 7 increase as compared to cells not contacted with the drug candidate.


SEQUENCE LISTING (27 sequences)

SEQ ID NO: 1 - protein, 401 aa, Ktedonobacter racemifer
MSTDATLIRTTPSHAEADATDTLVATPLMPPRRVISPWPGPGEGQSLMRIPVVDIRGMALMPCTPAKARHLLKSGNARPKRNKLGLFYVQLSYEQEPDNQSLVAGVDPGSKFEGLSVVGTKDTVLNLMVEAPDHVKGAVQTRRTMRRARRQRKWRRPKRFHNRLNRMQRIPPSTRSRWEAKARIVAHLRTILPFTDVVVEDVQAVTRKGKGGTWNGSFSPVQVGKEHLYRLLRAMGLTLHLREGWQTKELREQHGLKKTKSKSKQSFESHAVDSWVLAASISGAEHPTCTRLWYMVPAILHRRQLHRLQASKGGVRKPYGGTRSLGVKRGTLVEHKKYGRCTVGGVDRKRNTISLHEYRTNTRLTQAAKVETCRVLTWLSWRSWLLRGKRTSSKGKGSHSS

SEQ ID NO: 2 - protein, 432 aa, Oscillatoriales cyanobacterium
MQPAKQQNWVFQINGDKQPLDMINPGRCRELQNRGKLASFRRFPYVVIQQQTIENPQTKEYILKIDPGSQWTGFAIQCGNDILFRAELNHRGEAIKFDLVKRAWFRRGRRSRNLRYRKKRLNRAKPEGWLAPSIRHRVLTVETWIKRFMRYCPIAWIEIEQVRFDTQKLANPEIDGVEYQQGELQGYEVREYLLQKWGRKCAYCGTENVPLEVEHIQSKSKGGSSRIGNLTLACHVCNVKKGNLDVRDFLAKSPDILNQVLENSTKPLKDAAAVNSTRYAIVKMAKSICENVKCSSGARTKMNRVRQGLEKTHSLDAACVGESGASIRVLTDRPLLITCKGHGSRQSIRVNASGFPAVKNAKTVFTHIAAGDVVRFTIGKDRKKAQAGTYTARVKTPTPKGFEVLIDGARISLSTMSNVVFVHRSDGYGYEL

SEQ ID NO: 3 - protein, 461 aa, Acidihalobacter prosperus
MAVFVIDKHKRPLMPCSEKRARLLLERGRAVVHRQVPFVIRLKDRTVQHSAVQPLRVALDPGSRATGMALVREKNTVDTGTGEVYRERIALNLFELVHRGHRIREQLDQRRNFRRRRRGANLRYRAPRFDNRRRPPGWLAPSLQHRVDTTMAWVRRLCRWAPASAIGIETVRFDTQRLQNPEISGVEYQQGALAGCEVREYLLEKWGRKCAYCGAENVPLEIEHIVPKSRGGSDRVSNLALACRACNQAKGNRDVRAFLADQPERLARILAQAKAPLKDAAAVNATRWALYRALVDTGLPVEAGTGGRTKWNRTRLGLPKTHALDALCVGQVDQVRHWRVPVLGIRCAGRGSYRRTRLTRHGFPRGYLTRNKSAFGFQTGDLIRAVVTKGKKAGTYLGRIAIRASGSFNIQTPMGVVQGIHHRFCTLLQRADGYGYFVQPKPTEAALSSPRLKAGVSSAGN

SEQ ID NO: 4 - protein, 439 aa, Tolypothrix campylonemoides
MTTNVVFVIDTNQKPLQPCSAAVARKLLLRGKAAMFRRYPAVIILKKEVDSVGKPKIELRIDPGSKYTGFALVDSKDNADFIIWGTELEHRGAAICKELTKRSAIRRSRRNRKTRYRKKRFERRKPEGWLAPSLQHRVDTTLTWVKRICKFVPIMSISVEQVKFDLQKLENSDIQGIEYQQGTLAGYTLREALLEHWGRKCAYCDVENVFLEIEHIYPKSKGGSDKFSNLTLACHKCNINKGNKSIDEFLLSDHKRLEQIKLHQKKTLKDAAAVNATRKKLVTTLQEKTFLNVLVSDGASTKMTRLSSSLAKRHWIDAGCVNTTLIVILKTLQPLQVKCNGHGNKQFVTMDAYGFPRKSYEPKKVRKDWKAGDIIRVTKKDGTMLMGRVKKAAKKLVYIPFGGKEASFSSENAKAIHRSDGYRYSFAAIDSELLQKMAT

SEQ ID NO: 5 - protein, 419 aa, Halobacteroides halobius
MPNKYAFVLDSKGKLLDPTKSKKAWYLIRKGKASLVEEYPLIIKLKREVPKDQVNSDKLILGIDDGTKKVGFALVQKCQTKNKVLFKAVMEQRQDVSKKMEERRGYRRYRRSHKRYRPARFDNRSSSKRKGRIPPSILQKKQAILRVVNKLKKYIRIDKIVLEDVSIDIRKLTEGRELYNWEYQESNRLDENLRKATLYRDDCTCQLCGTTETMLHAHHIMPRRDGGADSIYNLITLCKACHKDKVDNNEYQYKDQFLAIIDSKELSDLKSASHVMQGKTWLRDKLSKIAQLEITSGGNTANKRIDYEIEKSHSNDAICTTGLLPVDNIDDIKEYYIKPLRKKSKAKIKELKCFRQRDLVKYTKRNGETYTGYITSLRIKNNKYNSKVCNFSTLKGKIFRGYGFRNLTLLNRPKGLMIV

SEQ ID NO: 6 - protein, 1350 aa, Streptococcus thermophilus
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERK

SEQ ID NO: 7 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 8 - protein, 1365 aa, Streptococcus pyogenes
KYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 9 - protein, 288 aa, Artificial Sequence
  Synthethic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 10 - protein, 183 aa, Artificial Sequence
  Synthethic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 11 - protein, 7 aa, Simian virus 40
PKKKRKV

SEQ ID NO: 12 - protein, 10 aa, Simian virus 40
PKKKRKVEAS

SEQ ID NO: 13 - protein, 16 aa, Artificial Sequence
  Synthethic
KRPAATKKAGQAKKKK

SEQ ID NO: 14 - protein, 9 aa, Homo sapiens
PAAKRVKLD

SEQ ID NO: 15 - protein, 11 aa, Homo sapiens
RQRRNELKRSP

SEQ ID NO: 16 - protein, 38 aa, Homo sapiens
NQSSNFGPMKGGNFGGRSSGPYGGGGQYFAKPRNQGGY

SEQ ID NO: 17 - protein, 42 aa, Homo sapiens
RMRIZFKNKGKDTAELRRRRVEVSVELRKAKKDEQILKRRNV

SEQ ID NO: 18 - protein, 8 aa, Homo sapiens
VSRKRPRP

SEQ ID NO: 19 - protein, 8 aa, Homo sapiens
PPKKARED

SEQ ID NO: 20 - protein, 8 aa, Homo sapiens
PQPKKKPL

SEQ ID NO: 21 - protein, 12 aa, Mus sp.
SALIKKKKKMAP

SEQ ID NO: 22 - protein, 5 aa, Influenza virus
DRLRR

SEQ ID NO: 23 - protein, 7 aa, Influenza virus
PKQKKRK

SEQ ID NO: 24 - protein, 10 aa, Hepatitis D virus
RKLKKKIKKL

SEQ ID NO: 25 - protein, 10 aa, Mus sp.
REKKKFLKRR

SEQ ID NO: 26 - protein, 20 aa, Homo sapiens
KRKGDEVDGVDEVAKKKSKK

SEQ ID NO: 27 - protein, 17 aa, Homo sapiens
RKCLQAGMNLEARKTKK